(19) United States
(12) Patent Application Publication
(10) Pub. No.: US 2026/0274797 A1
(43) Pub. Date: Sep. 17, 2026

(54) PROPIONIC ACID DERIVATIVE AND USE THEREOF IN MEDICINE

(71) Applicant: Xizang Haisco Pharmaceutical Co., Ltd., Lhoka, Tibet (CN)

(72) Inventors: Chen ZHANG, Lhoka, Tibet (CN); Ping HE, Lhoka, Tibet (CN); Le WANG, Lhoka, Tibet (CN); Zhaoli XUAN, Lhoka, Tibet (CN); Qi WEI, Lhoka, Tibet (CN); Pingming TANG, Lhoka, Tibet (CN); Haiqing HE, Lhoka, Tibet (CN); Yajun ZHONG, Lhoka, Tibet (CN); Yan YU, Lhoka, Tibet (CN); Yao LI, Lhoka, Tibet (CN); Jia NI, Lhoka, Tibet (CN); Pangke YAN, Lhoka, Tibet (CN)

(21) Appl. No.: 19/110,248

(22) PCT Filed: Sep. 8, 2023
(86) PCT No.: PCT/CN2023/117732

§ 371 Date: Mar. 10, 2025

(30) Foreign Application Priority Data
Sep. 9, 2022 (CN) 202211104699.7
Nov. 11, 2022 (CN) 202211391154.9
Jan. 5, 2023 (CN) 202310000075.9
Feb. 23, 2023 (CN) 202310157111.2
May 16, 2023 (CN) 202310546078.2
Jul. 21, 2023 (CN) 202310900169.1

(51) Int. Cl. C07D 213/64; A61K 31/4412; A61K 31/4427; A61K 31/443; A61K 31/4439; A61K 31/4545; A61K 31/501; A61K 31/513; A61K 31/553; C07B 59/00; C07D 401/04; C07D 401/06; C07D 401/12; C07D 403/06; C07D 405/12

(52) U.S. Cl. CPC C07D 213/64; A61K 31/4412; A61K 31/4427; A61K 31/443; A61K 31/4439; A61K 31/4545; A61K 31/501; A61K 31/513; A61K 31/553; C07B 59/002; C07D 401/04; C07D 401/06; C07D 401/12; C07D 403/06; C07D 405/12

(57) ABSTRACT

A compound of general formula (I) or a stereoisomer, mesomer/racemate, deuterated product, solvate, prodrug, metabolite, pharmaceutically acceptable salt, or co-crystal thereof, an intermediate thereof, a preparation method therefor, and a use in the preparation of a drug for treating diseases related to integrin α4β7 activity or expression.

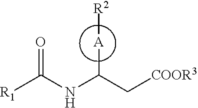

### CROSS-REFERENCE TO RELATED APPLICATION

This application is a 35 U.S.C. § 371 National Stage of International Patent Application No. PCT/CN2023/117732, filed Sep. 8, 2023, designating the United States, which claims priority to and the benefits of Chinese Patent Application No. 202211104699.7, filed Sep. 9, 2022, Chinese Patent Application No. 202211391154.9, filed Nov. 11, 2022, Chinese Patent Application No. 202310000075.9, filed Jan. 5, 2023, Chinese Patent Application No. 202310157111.2, filed Feb. 23, 2023, Chinese Patent Application No. 202310546078.2, filed May 16, 2023, and Chinese Patent Application No. 202310900169.1, filed Jul. 21, 2023, the disclosures of which are incorporated herein in their entirety by reference, and priority is claimed to each of the foregoing.

### TECHNICAL FIELD

The present disclosure relates to a compound of general formula (I) or a stereoisomer, mesomer/racemate, deuterated product, solvate, prodrug, metabolite, pharmaceutically acceptable salt, or co-crystal thereof, an intermediate thereof, a preparation method therefor, and a use in the preparation of a drug for treating diseases related to integrin α4β7 activity or expression.

### BACKGROUND

The integrin family comprises dimers formed by two subunits: α (120-185 KD) and β (90-110 KD). Mammalian integrins involve 18 α-subunits and 8 β-subunits. More than 20 integrins can be formed from their various combinations. α4β7 is a member of the integrin family. It is currently established that inflammatory bowel diseases related to α4β7 include Crohn's disease, ulcerative colitis, etc. The main ligand of α4β7 is mucosal addressin cell adhesion molecule-1 (MADCAM-1). MAdCAM-1 is a transmembrane glycoprotein molecule selectively expressed in the high endothelial veins of mucosal lymphoid organs and the veins of the intestinal lamina propria. In inflammation, a variety of cytokines can promote high expression of MAdCAM-1 by endothelial cells, and then MAdCAM-1 mediates the migration of α4β7-expressing leukocytes to the site of inflammation. Targeting either integrin α4β7 or MAdCAM-1 can reduce intestinal inflammation. There are currently no specific small-molecule compounds on the market that target α4β7-mediated inflammation. Natalizumab in clinical use is a humanized monoclonal antibody that targets the α4 subunit and is used primarily to treat multiple sclerosis and Crohn's disease. However, the side effect of PML (progressive multifocal leukoencephalopathy) has occurred during its clinical use. Therefore, there is a need to develop a small-molecule compound, which can inhibit integrin α4β7 protein, for use in the treatment of diseases related to an integrin α4β7 activity or expression level.

### SUMMARY

The present disclosure develops an integrin α4β7 inhibitor with novel structure, good drug efficacy, better selectivity for α4β1/α4β7, better stability of liver microsomes, greater safety, and no significant inhibitory activity against hERG or CYP. These compounds have good pharmacokinetic properties and good safety and can be used to treat diseases related to integrin α4β7, such as inflammatory bowel diseases.

An object of the present disclosure is to provide a compound capable of inhibiting integrin α4β7, or a stereoisomer, mesomer/racemate, deuterated product, solvate, prodrug, metabolite, pharmaceutically acceptable salt, or co-crystal thereof, an intermediate thereof, a preparation method therefor, and a use in the preparation of a drug for the treatment of diseases related to integrin α4β7 activity or expression.

The present disclosure provides a compound of general formula (I) or a stereoisomer, mesomer/racemate, deuterated product, solvate, prodrug, metabolite, pharmaceutically acceptable salt, or co-crystal thereof, wherein

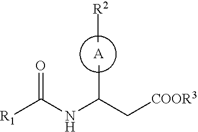

in some embodiments, R¹ is selected from —CHR^{1a}R^{1b} or —NR^{1a}R^{1b};
in some embodiments, R¹ is selected from

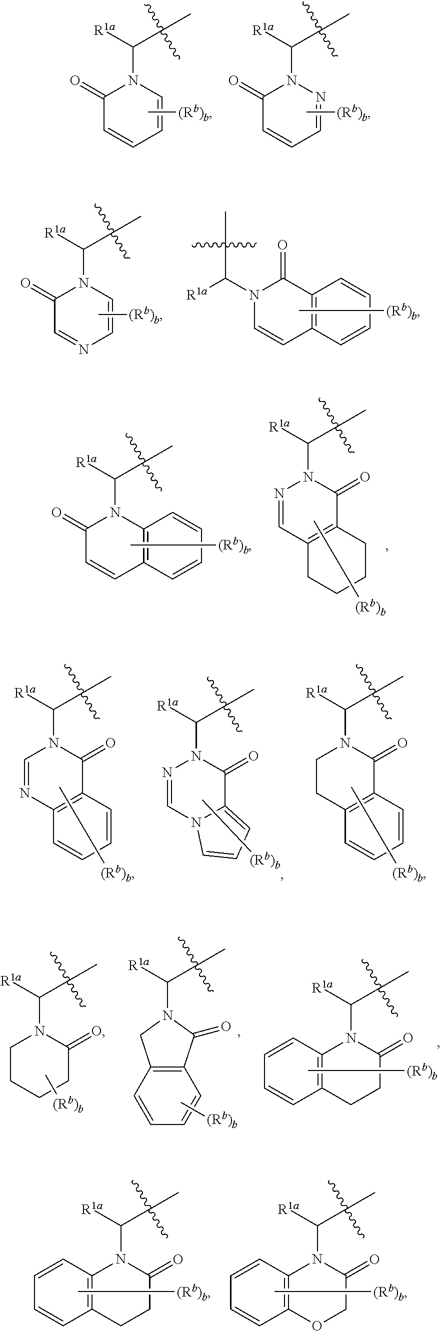

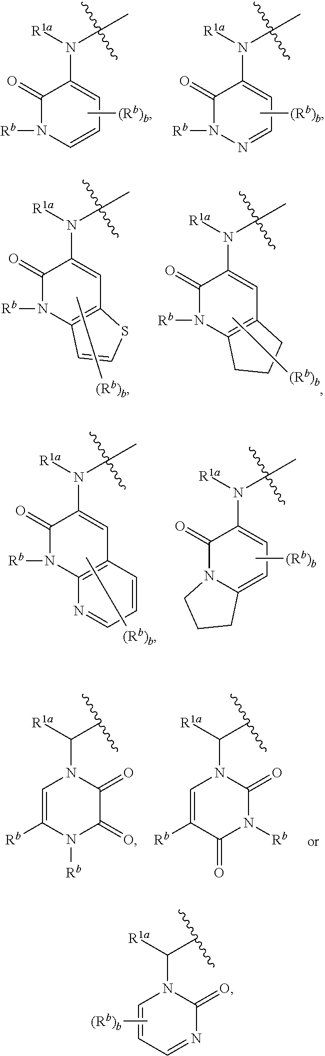

and b is selected from 0, 1, 2 or 3;
in some embodiments, R¹ is selected from

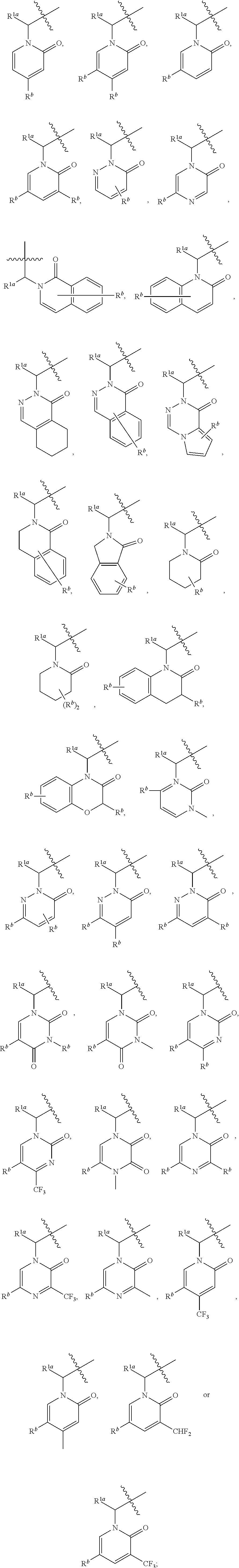

in some embodiments, R¹ is selected from

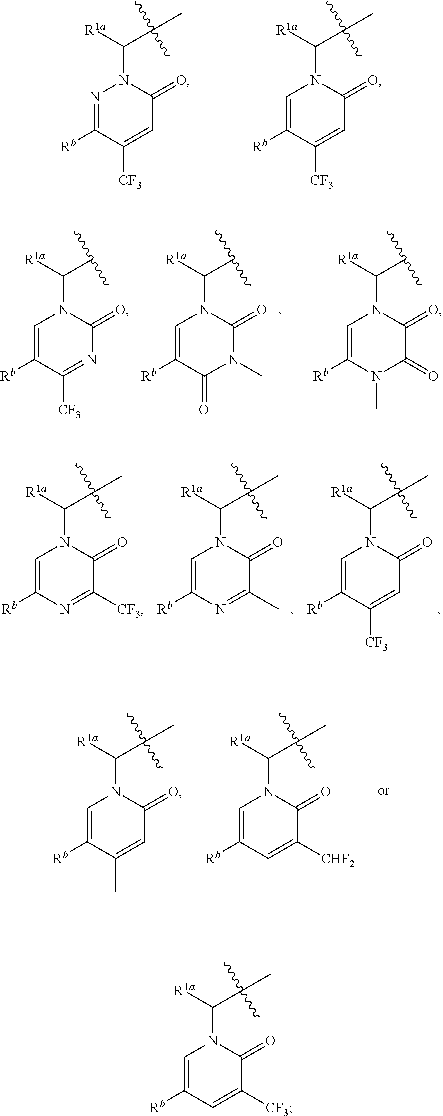

in some embodiments, R¹ is selected from

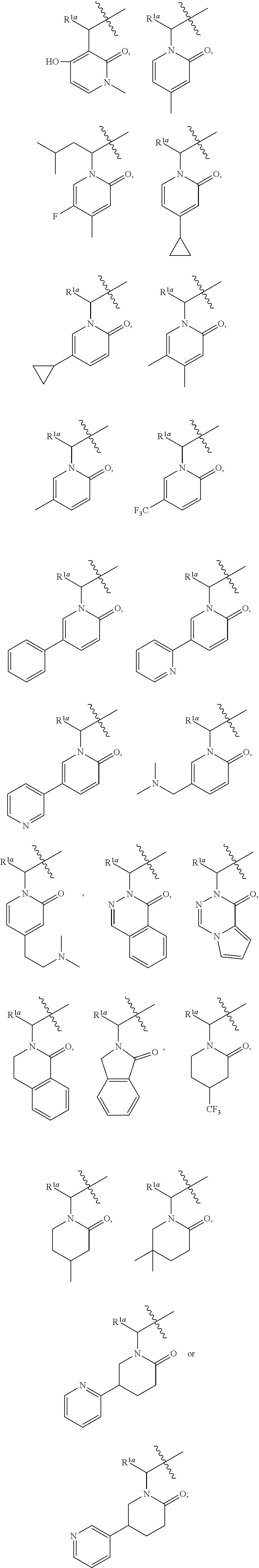

in some embodiments, R¹ is selected from

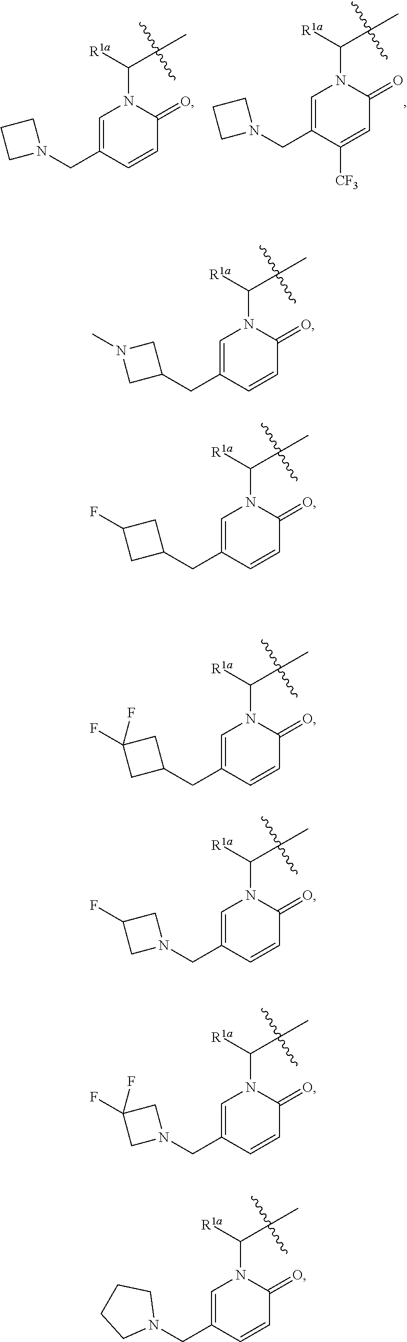

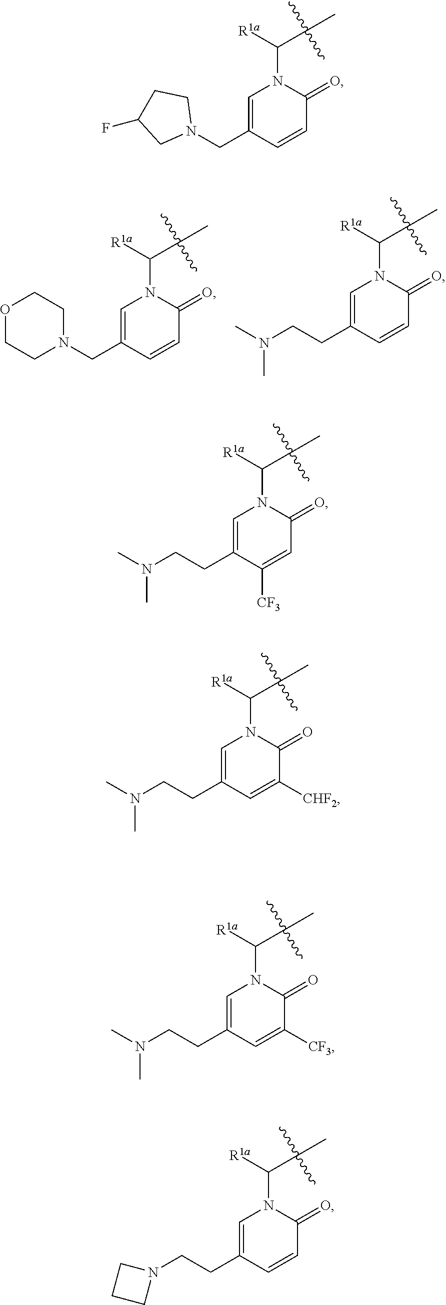

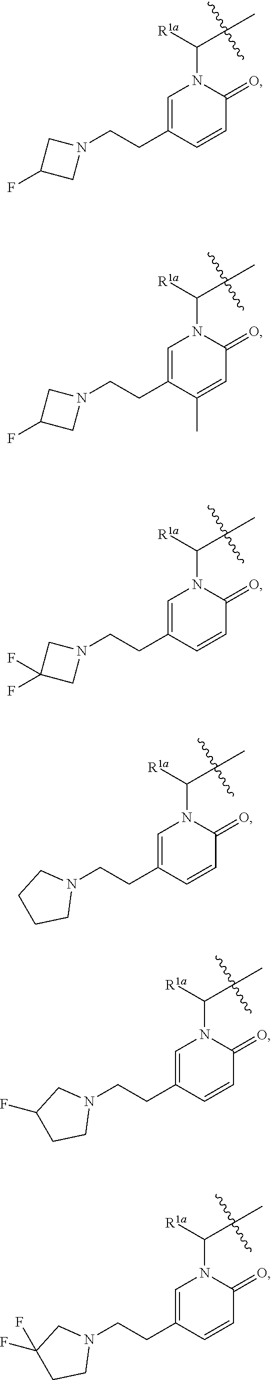

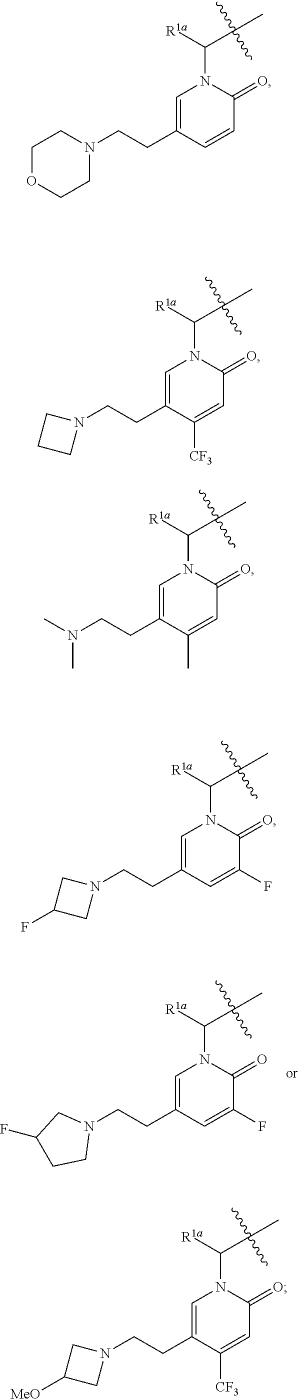

in some embodiments, R¹ is selected from

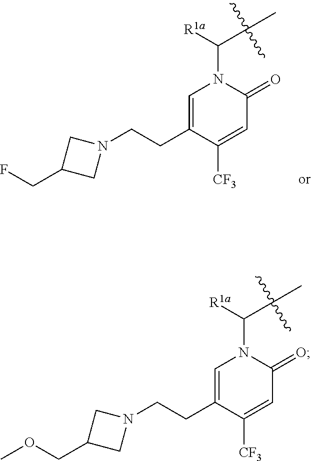

in some embodiments, R¹ is selected from

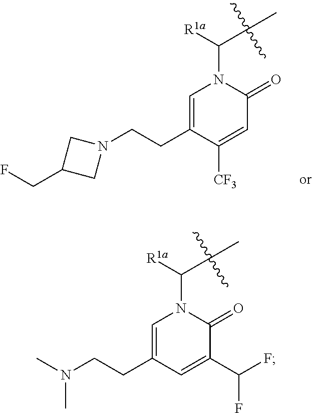

in some embodiments, R¹ is selected from

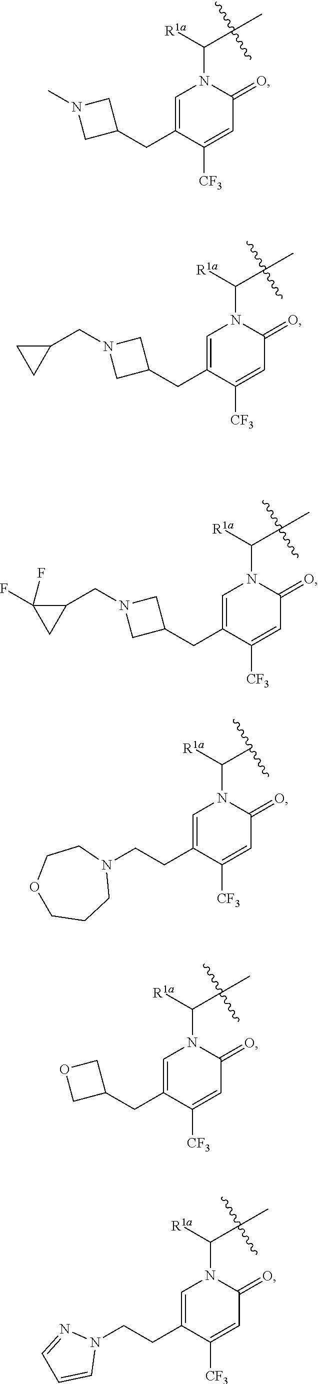

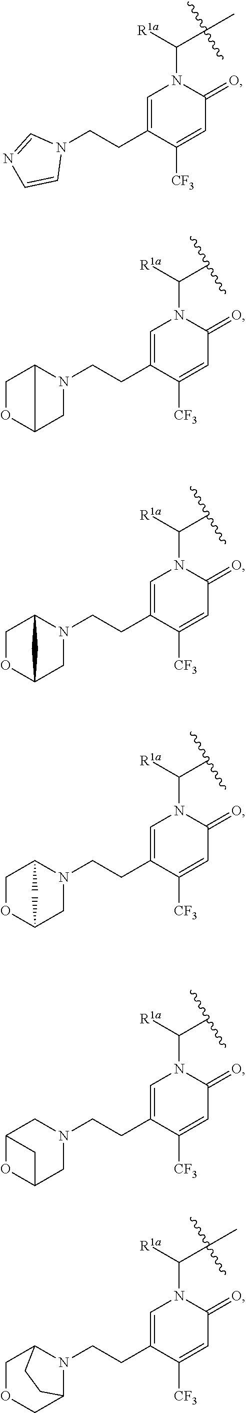

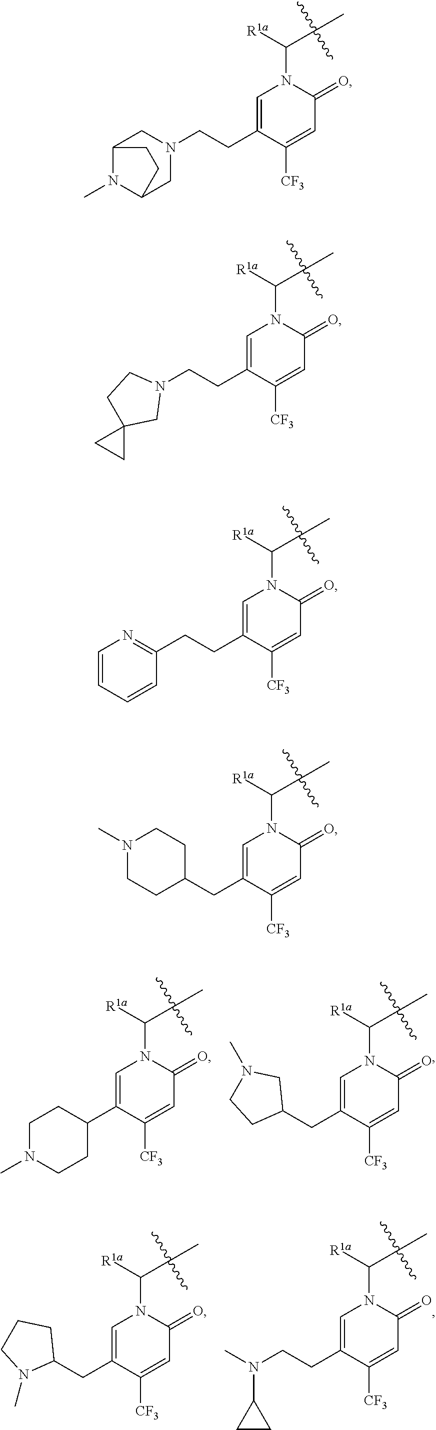

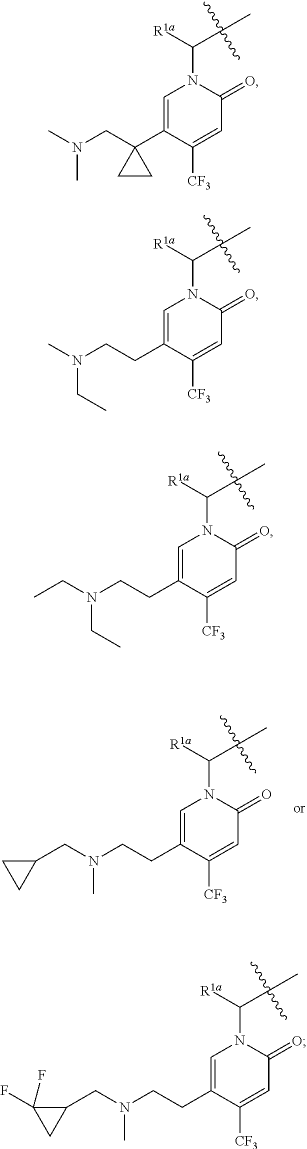

in some embodiments, R¹ is selected from

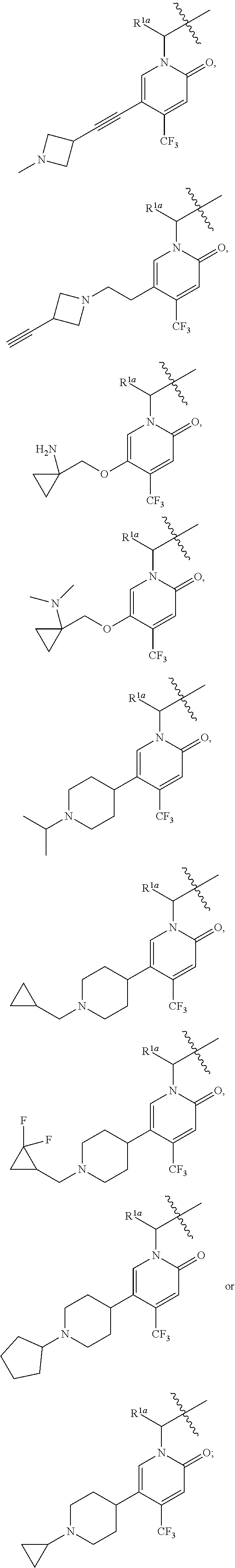

in some embodiments, R¹ is selected from

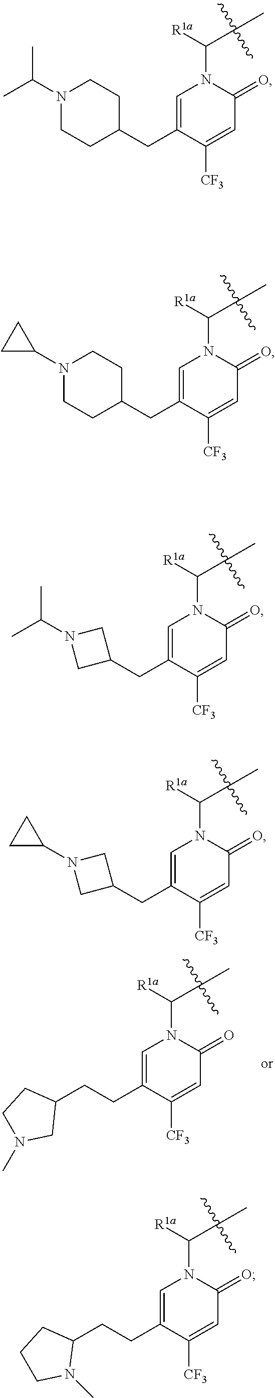

in some embodiments, R¹ is selected from

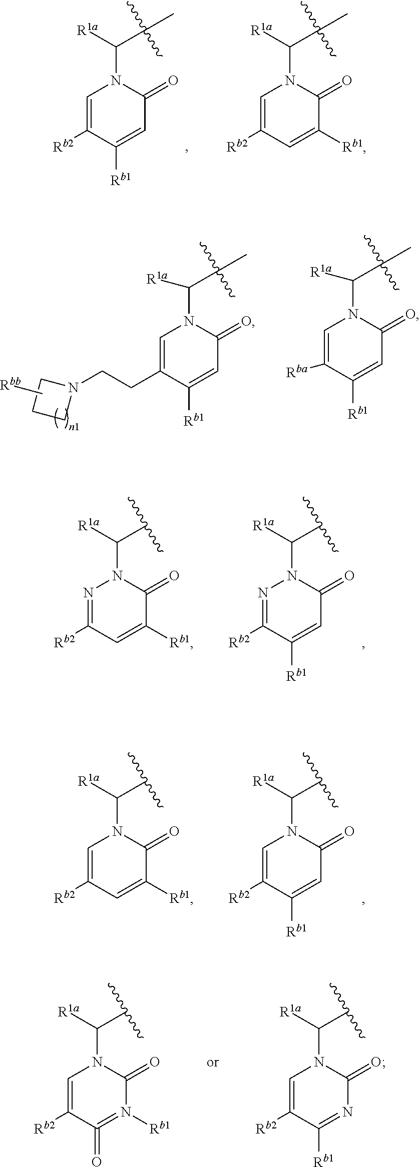

in some embodiments, R¹ is selected from

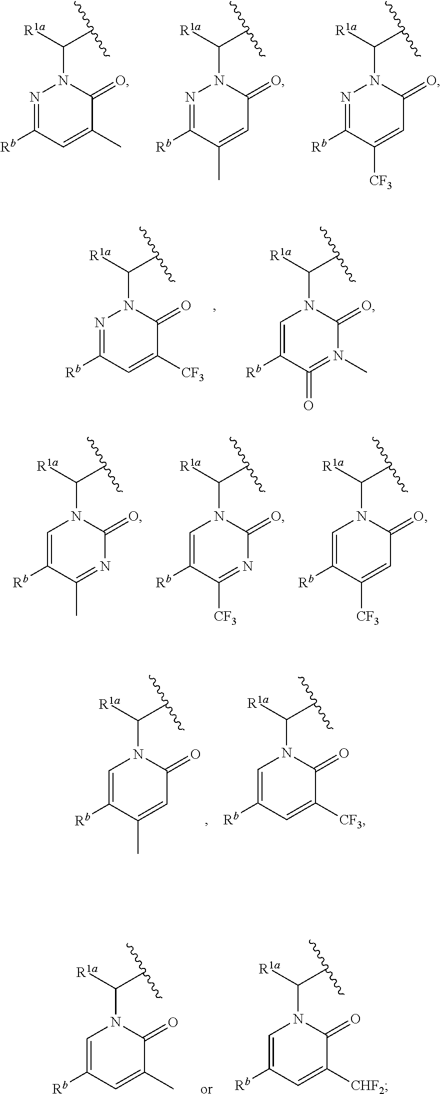

in some embodiments, R^{b1} is selected from H, F, CH₂F, CHF₂, CF₃, methyl or —CH₂CH₂N(CH₃)₂;
in some embodiments, R^{b2} and R^{b} are each independently selected from R^{ba},

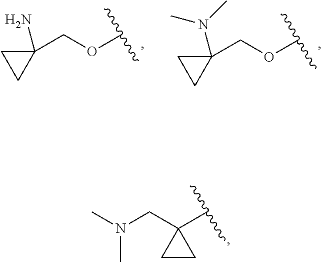

or one of the following optionally substituted groups: ethynyl, cyclopropyl, cyclobutyl, cyclopentyl, cyclohexyl, azetidinyl, azolidinyl, azinanyl, oxetanyl, oxolanyl, oxanyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azolidinyl, —CH₂-azinanyl, —CH₂-oxetanyl, —CH₂-oxolanyl, —CH₂-oxanyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl, —CH₂CH₂-morpholinyl, —CH₂-phenyl, —CH₂-pyridyl, —CH₂CH₂-phenyl, —CH₂CH₂-pyridyl or

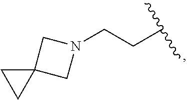 [SMILES: *CCN1CC2(CC2)C1]

which, when substituted, is substituted with 1 to 4 substituents selected from deuterium, F, Cl, Br, OH, ═O, cyano, CONH₂, CONHCH₃, CON(CH₃)₂, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
in some embodiments, R^{b2} and R^{b} are each independently selected from R^{ba},

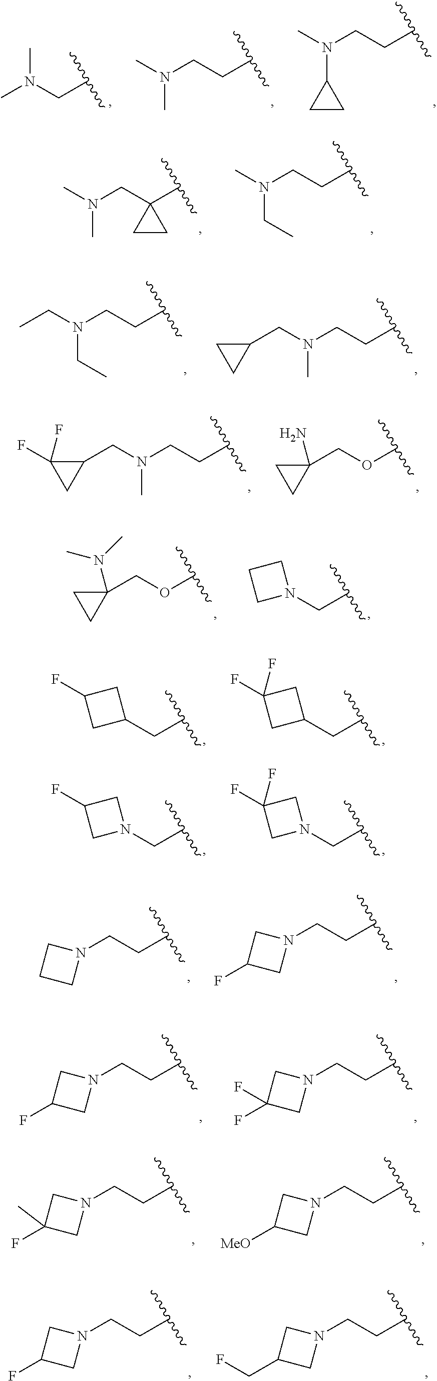

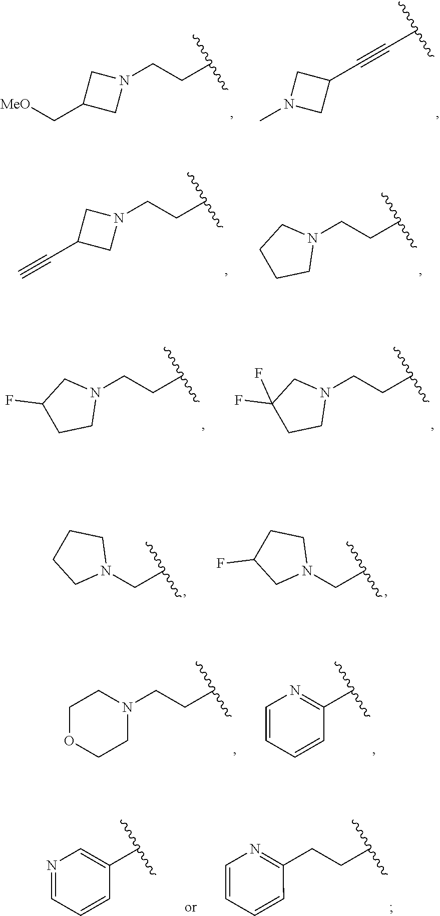

in some embodiments, R^{ba} is selected from

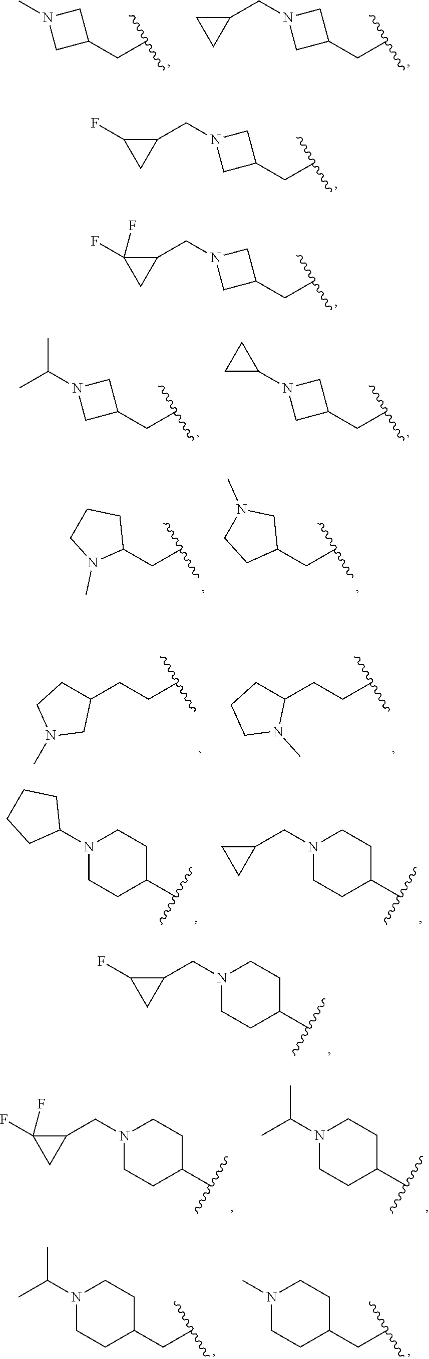

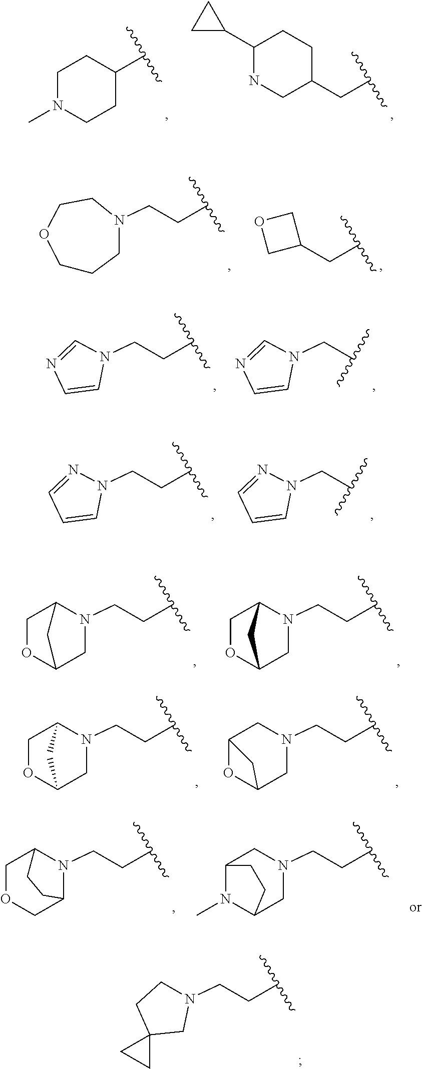

in some embodiments, R^{b1} is selected from H, C₁₋₄ alkyl or C₃₋₆ cycloalkyl, wherein the alkyl or cycloalkyl is optionally substituted with 0 to 4 substituents selected from deuterium, halogen, OH, CN, NHC₁₋₄ alkyl, N(C₁₋₄ alkyl)₂, C₁₋₄ alkyl or C₁₋₄ alkoxy;
in some embodiments, R^{b2} is selected from H, R^{ba}, or one of the following substituted or unsubstituted groups: C₁₋₄ alkyl, C₂₋₄ alkynyl, C₁₋₄ alkoxy, —CH₂CH₂—NHC₁₋₄ alkyl, —CH₂CH₂—N(C₁₋₄ alkyl)₂, —CH₂CH₂—C₃₋₆ cycloalkyl, —CH₂CH₂-3- to 7-membered heterocycloalkyl, wherein the CH₂, alkyl, cycloalkyl or heterocycloalkyl is optionally substituted with 0 to 4 substituents selected from deuterium, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
in some embodiments, R^{1a} is selected from C₁₋₆ alkyl, wherein the alkyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl;
in some embodiments, R^{1a} is selected from methyl, ethyl, propyl, butyl, isobutyl, sec-butyl, tert-butyl, —CH₂-cyclopropyl or —CH₂-cyclobutyl;
in some embodiments, R^{1a} is selected from

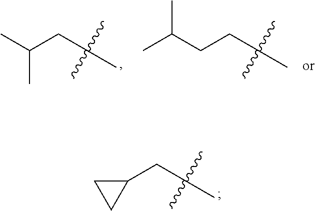

in some embodiments, R^{1b} is selected from C₄₋₁₀ carbocyclic ring or 5- to 10-membered heterocyclic ring, wherein the carbocyclic ring or heterocyclic ring is optionally substituted with 0 to 4 R^{b}, and the heterocyclic ring contains 1 to 4 heteroatoms selected from O, S or N;
in some embodiments, each R^{b} is independently selected from deuterium, halogen, OH, ═O, cyano, COOH, NH₂, —C₀₋₄ alkyl-NHC₁₋₆ alkyl, —C₀₋₄ alkyl-N(C₁₋₆ alkyl)₂, C₁₋₆ alkyl, C₂₋₆ alkynyl, C₁₋₆ alkoxy, —C₀₋₄ alkyl-C₃₋₁₀ carbocyclic ring, —C₀₋₄ alkyl-3- to 10-membered heterocyclic ring or R^{ba}, wherein the alkyl, alkynyl, alkoxy, carbocyclic ring or heterocyclic ring is optionally substituted with 0 to 4 substituents selected from deuterium, halogen, OH, ═O, cyano, COOH, CONH₂, CONHC₁₋₆ alkyl, CON(C₁₋₆ alkyl)₂, NH₂, NHC₁₋₆ alkyl, N(C₁₋₆ alkyl)₂, N(C₁₋₆ alkyl)(C₃₋₆ cycloalkyl), NH(C₃₋₆ cycloalkyl), C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, and the heterocyclic ring contains 1 to 4 heteroatoms selected from O, S or N;
in some embodiments, each R^{b} is independently selected from deuterium, halogen, OH, ═O, cyano, COOH, NH₂, —C₀₋₄ alkyl-NHC₁₋₄ alkyl, —C₀₋₄ alkyl-N(C₁₋₄ alkyl)₂, C₁₋₄ alkyl, C₁₋₄ alkoxy, —C₀₋₄ alkyl-C₃₋₆ carbocyclic ring, —C₀₋₄ alkyl-3- to 7-membered heterocyclic ring or R^{ba}, wherein the alkyl, alkoxy, carbocyclic ring or heterocyclic ring is optionally substituted with 0 to 4 substituents selected from deuterium, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heterocyclic ring contains 1 to 4 heteroatoms selected from O, S or N;
in some embodiments, R^{b} is selected from R^{ba};
In some embodiments, each R^{b} is independently selected from deuterium, halogen, OH, ═O, cyano, C₁₋₄ alkyl, C₂₋₄ alkynyl, C₁₋₄ alkoxy, phenyl, 5- to 6-membered heteroaryl, —CH₂NHC₁₋₄ alkyl, —CH₂N(C₁₋₄ alkyl)₂, —CH₂CH₂—NHC₁₋₄ alkyl, —CH₂CH₂—N(C₁₋₄ alkyl)₂, C₃₋₆ cycloalkyl, 3- to 7-membered heterocycloalkyl, —CH₂—C₃₋₆ cycloalkyl, —CH₂-3- to 7-membered heterocycloalkyl, —CH₂CH₂—C₃₋₆ cycloalkyl, —CH₂CH₂-3- to 7-membered heterocycloalkyl or R^{ba}, wherein the CH₂, alkyl, alkynyl, cycloalkyl or heterocycloalkyl is optionally substituted with 0 to 4 substituents selected from deuterium, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
in some embodiments, each R^{b} is independently selected from deuterium, F, Cl, Br, I, OH, ═O, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azolidinyl, azinanyl, oxetanyl, oxolanyl, oxanyl, morpholinyl, phenyl, pyridine, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azolidinyl, —CH₂-azinanyl, —CH₂-oxetanyl, —CH₂-oxolanyl, —CH₂-oxanyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl or —CH₂CH₂-morpholinyl, which, when substituted, is substituted with 0 to 4 substituents selected from deuterium, halogen, OH, ═O, cyano, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
in some embodiments, each R^{b} is independently selected from deuterium, F, Cl, Br, OH, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azolidinyl, azinanyl, oxetanyl, oxolanyl, oxanyl, morpholinyl, phenyl, pyridine, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azolidinyl, —CH₂-azinanyl, —CH₂-oxetanyl, —CH₂-oxolanyl, —CH₂-oxanyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl or —CH₂CH₂-morpholinyl, which, when substituted, is substituted with 0 to 4 substituents selected from deuterium, F, Cl, Br, OH, ═O, cyano, CONH₂, CONHCH₃, CON(CH₃)₂, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
in some embodiments, R^{b} is selected from R^{b2};
in some embodiments, R^{ba} is selected from —C₀₋₄ alkyl-7- to 12-membered heterocyclic ring, —C₀₋₄ alkyl-4- to 6-membered heterocyclic ring connected via a carbon atom,

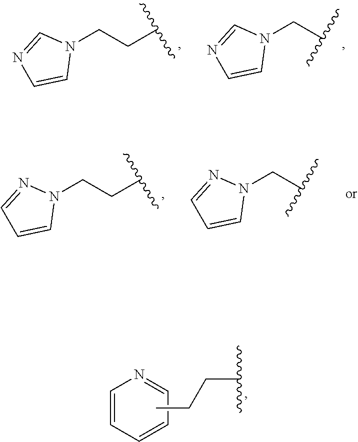

and the R^{ba} is optionally substituted with 1 to 4 substituents selected from H, halogen, OH, ═O, cyano, COOH, CONH₂, CONHC₁₋₆ alkyl, CON(C₁₋₆ alkyl)₂, NH₂, NHC₁₋₆ alkyl, N(C₁₋₆ alkyl)₂, C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, wherein the heterocyclic ring contains 1 to 4 heteroatoms selected from O, S or N;
in some embodiments, R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl connected via a carbon atom,

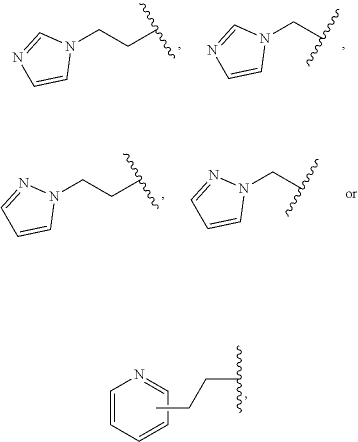

and the R^{ba} is optionally substituted with 1 to 4 substituents selected from H, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
in some embodiments, R^{ba} is selected from 7- to 8-membered monocyclic heterocycloalkyl, 7- to 11-membered spiro heterocycloalkyl, 7- to 11-membered bridged heterocycloalkyl, 4- to 6-membered monocyclic heterocycloalkyl connected via a carbon atom, —CH₂-7- to 8-membered monocyclic heterocycloalkyl, —CH₂-7- to 11-membered spiro heterocycloalkyl, —CH₂-7- to 11-membered bridged heterocycloalkyl, —CH₂-4- to 6-membered monocyclic heterocycloalkyl connected via a carbon atom, —CH₂CH₂-7- to 8-membered monocyclic heterocycloalkyl, —CH₂CH₂-7- to 11-membered spiro heterocycloalkyl, —CH₂CH₂-7- to 11-membered bridged heterocycloalkyl, —CH₂CH₂-4- to 6-membered monocyclic heterocycloalkyl connected via a carbon atom,

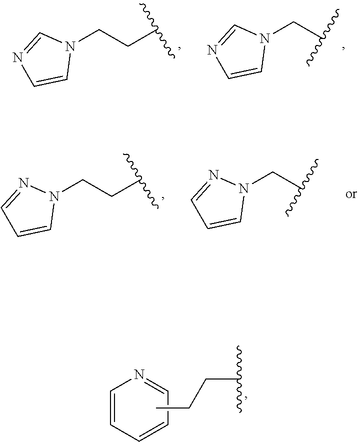

and the R^{ba} is optionally substituted with 1 to 4 substituents selected from H, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
in some embodiments, R^{ba} is selected from one of the following optionally substituted groups:

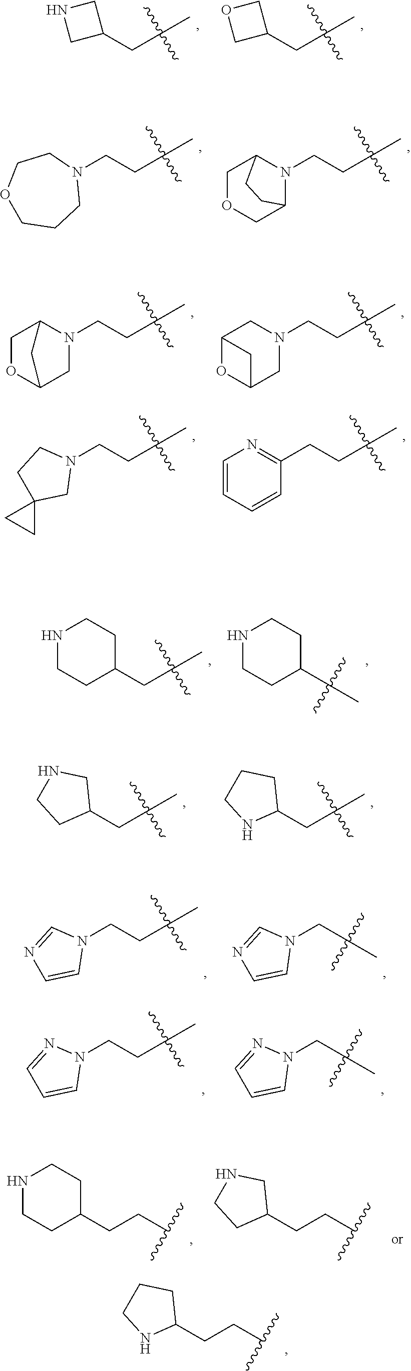

which, when substituted, is optionally substituted with 1 to 4 substituents selected from deuterium, halogen, OH, ═O, cyano, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
in some embodiments, R^{ba} is selected from one of the following optionally substituted groups:

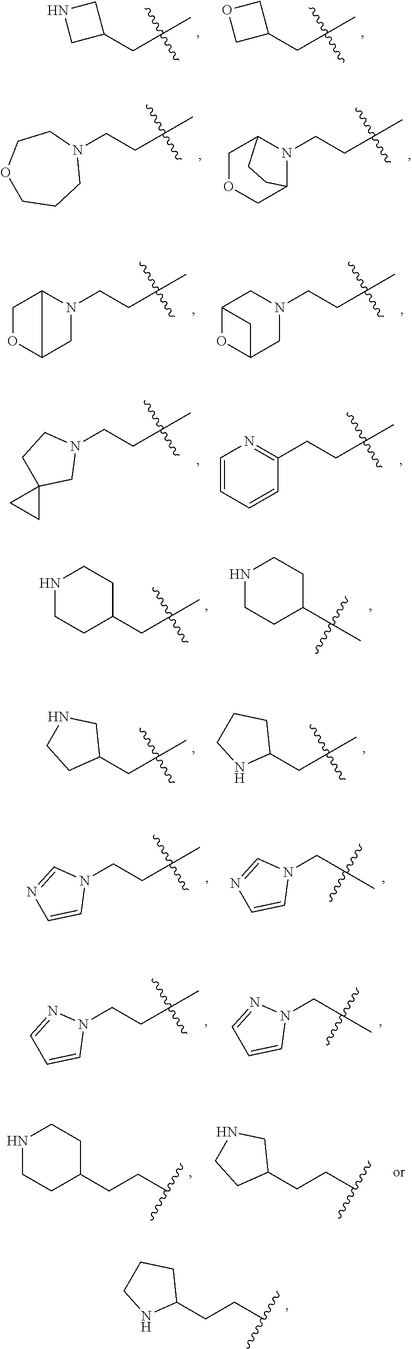

which, when substituted, is optionally substituted with 1 to 4 substituents selected from deuterium, F, Cl, Br, OH, ═O, cyano, CONH₂, CONHCH₃, CON(CH₃)₂, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
in some embodiments, R^{k} is selected from —C₁₋₄ alkyl-NH₂, —C₁₋₄ alkyl-NHC₁₋₆ alkyl, —C₁₋₄ alkyl-N(C₁₋₆ alkyl)₂, —C₀₋₄ alkyl-C₃₋₁₀ carbocyclic ring or —C₀₋₄ alkyl-3- to 10-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy, halogen-substituted C₁₋₆ alkyl, halogen-substituted C₁₋₆ alkoxy or C₁₋₆ alkoxyalkyl, and the heterocyclic ring contains 1 to 4 heteroatoms selected from O, S or N;
in some embodiments, R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclic ring or —C₀₋₂ alkyl-3- to 6-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclic ring contains 1 to 4 heteroatoms selected from O, S or N;
in some embodiments, R^{k} is selected from —C₁₋₄ alkyl-NH₂, —C₁₋₄ alkyl-NHC₁₋₄ alkyl, —C₁₋₄ alkyl-N(C₁₋₄ alkyl)₂, —C₀₋₄ alkyl-C₃₋₆ carbocyclic ring or —C₀₋₄ alkyl-3- to 6-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclic ring contains 1 to 4 heteroatoms selected from O, S or N;
in some embodiments, R^{k} is selected from —CH₂N(CH₃)₂, —CH₂-cyclopropyl, —CH₂-cyclobutyl, cyclopropyl, cyclobutyl, cyclopentyl, cyclohexyl, azetidinyl, azolidinyl, azinanyl or oxetanyl, wherein the cyclopropyl, cyclobutyl, cyclopentyl, cyclohexyl, azetidinyl, azolidinyl, azinanyl or oxetanyl is optionally substituted with 1 to 4 substituents selected from deuterium, halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₁₋₄ alkoxy, halogen-substituted C₁₋₄ alkyl, halogen-substituted C₁₋₄ alkoxy or C₁₋₄ alkoxyalkyl;
in some embodiments, R^{k} is selected from —CH₂N(CH₃)₂, —CH₂-cyclopropyl, —CH₂-cyclobutyl, cyclopropyl, cyclobutyl, cyclopentyl, cyclohexyl, azetidinyl, azolidinyl, azinanyl or oxetanyl, wherein the cyclopropyl, cyclobutyl, cyclopentyl, cyclohexyl, azetidinyl, azolidinyl, azinanyl or oxetanyl is optionally substituted with 1 to 4 substituents selected from deuterium, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, methoxy, ethoxy, methoxymethyl, ethoxymethyl or methoxyethyl;
in some embodiments, R² is selected from C₁₋₆ alkyl, C₆₋₁₀ aromatic ring, 5- to 10-membered heteroaromatic ring, C₃₋₁₀ carbocyclic ring or 5- to 10-membered heterocyclic ring, and the R² is optionally substituted with 0 to 4 R^{2a}, wherein the heteroaromatic ring or heterocyclic ring contains 1 to 4 heteroatoms selected from O, S or N;
in some embodiments, R² is selected from C₁₋₄ alkyl, benzene ring, naphthalene ring, 5- to 6-membered heteroaromatic ring, 9- to 10-membered heteroaromatic ring, C₃₋₁₀ non-aromatic carbocyclic ring, 5- to 10-membered non-aromatic heterocyclic ring, benzo C₄₋₆ carbocyclyl or benzo 4- to 6-membered heterocyclyl, and the R² is optionally substituted with 0 to 4 R^{2a}, wherein the heteroaromatic ring or heterocyclic ring contains 1 to 4 heteroatoms selected from O, S or N;
in some embodiments, R² is selected from benzene ring, naphthalene ring, 5- to 6-membered heteroaromatic ring, 9- to 10-membered heteroaromatic ring, C₃₋₆ cycloalkyl, 3- to 7-membered heterocycloalkyl, benzo C₄₋₆ carbocyclyl or benzo 4- to 6-membered heterocyclyl, and the R² is optionally substituted with 0 to 4 R^{2a}, wherein the heteroaromatic ring or heterocycloalkyl contains 1 to 4 heteroatoms selected from O, S or N; in some embodiments, R² is selected from benzene ring, pyridyl, pyridonyl, pyrazinyl, pyrimidyl, thienyl, thiazolyl, furyl, oxazolyl, pyrrolyl, pyrazolyl, imidazolyl, cyclopropyl, cyclobutyl, cyclopentyl, cyclohexyl, azetidinyl, azolidinyl, azinanyl, oxetanyl, oxolanyl, oxanyl, morpholinyl,

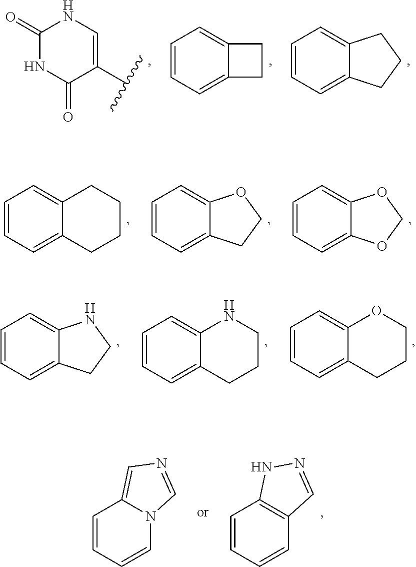

and the R² is optionally substituted with 0 to 4 R^{2a}; in some embodiments, R² is selected from

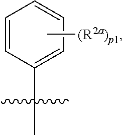

wherein p1 is selected from 0, 1, 2, 3 or 4;
in some embodiments, R² is selected from

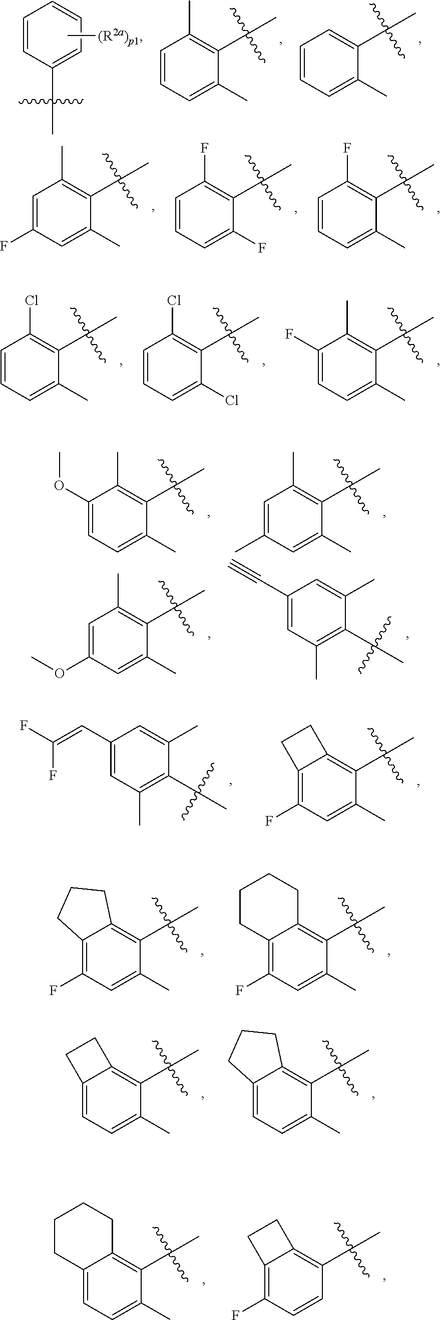

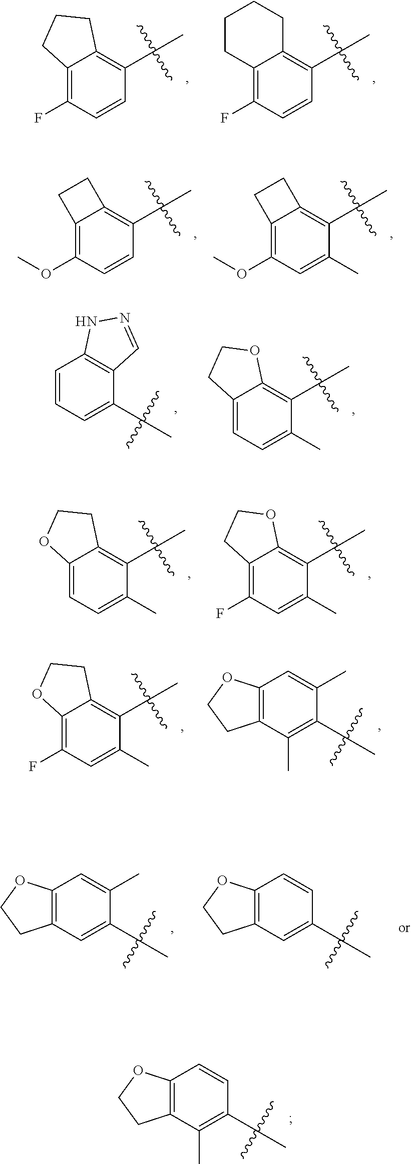

in some embodiments, R² is selected from phenyl, pyridyl, pyridonyl, azolidinyl, morpholinyl or

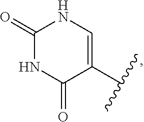 [SMILES: *c1c[nH]c(=O)[nH]c1=O]

and the R² is optionally substituted with 0 to 4 substituents selected from deuterium, F, Cl, Br, OH, CF₃, cyano, methyl, ethyl, methoxy, ethoxy, cyclopropyl or cyclobutyl;
in some embodiments, R² is selected from

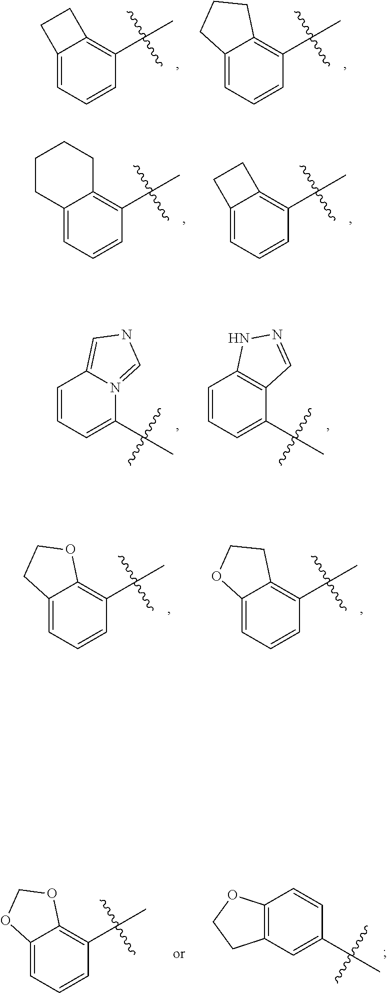

and the R² is optionally substituted with 1 to 4 substituents selected from deuterium, CD₃, —OCD₃, F, Cl, Br, OH, CF₃, cyano, methyl, ethyl, methoxy, ethoxy, cyclopropyl or cyclobutyl;
in some embodiments, R² is selected from

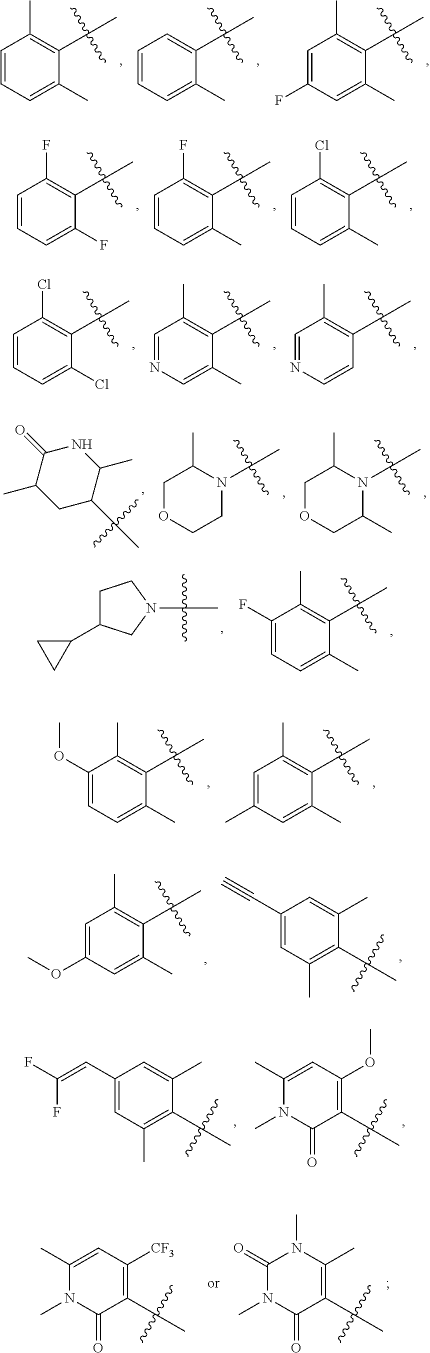

in some embodiments, R² is selected from

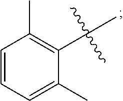 [SMILES: *c1c(C)cccc1C]

in some embodiments, R² is selected from

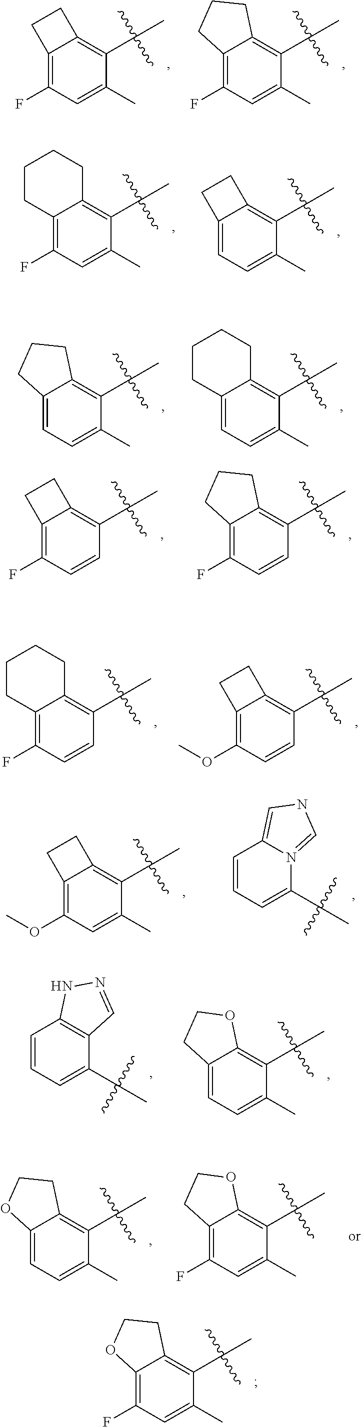

in some embodiments, R² is selected from

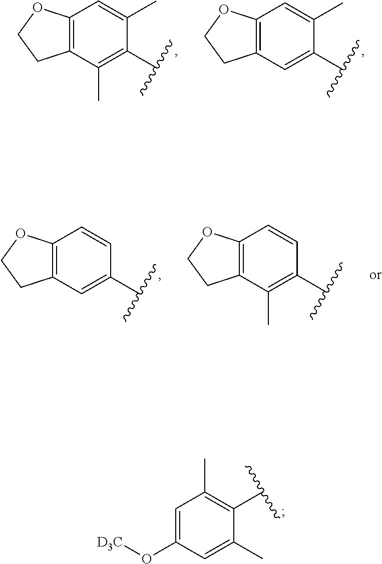 [SMILES: *c1c(C)cc2c(c1C)CCO2 ; *c1cc2c(cc1C)OCC2 ; *c1ccc2c(c1)CCO2 ; *c1ccc2c(c1C)CCO2 ; *c1c(C)cc(OC([2H])([2H])[2H])cc1C]

in some embodiments, each R^{2a} is independently selected from deuterium, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
in some embodiments, each R^{2a} is independently selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
in some embodiments, R^{2a} is selected from deuterium, halogen, OH, CN, C₁₋₄ alkyl, C₂₋₄ alkenyl, C₂₋₄ alkynyl, C₁₋₄ alkoxy, C₃₋₆ cycloalkyl or 4- to 6-membered heterocycloalkyl, wherein the alkyl, alkenyl, alkynyl, alkoxy, cycloalkyl or heterocycloalkyl is optionally substituted with 0 to 4 substituents selected from deuterium, halogen, OH, CN, C₁₋₄ alkyl, or C₁₋₄ alkoxy, and the heterocycloalkyl contains 1 to 4 heteroatoms selected from O, S or N;
in some embodiments, each R^{2a} is independently selected from deuterium, halogen, OH, cyano, ═O, C₁₋₄ alkyl, C₂₋₄ alkenyl, C₂₋₄ alkynyl, C₁₋₄ alkoxy or C₃₋₆ cycloalkyl, wherein the alkyl, alkenyl, alkynyl, alkoxy or cycloalkyl is optionally substituted with 0 to 4 substituents selected from deuterium, halogen, OH, ═O, cyano, C₁₋₄ alkyl or C₁₋₄ alkoxy;
in some embodiments, each R^{2a} is independently selected from deuterium, F, Cl, Br, I, OH, ═O, cyano, methyl, ethyl, ethenyl, ethynyl, methoxy, ethoxy, cyclopropyl or cyclobutyl, wherein the methyl, ethyl, ethenyl, ethynyl, methoxy, ethoxy, cyclopropyl or cyclobutyl is optionally substituted with 0 to 4 substituents selected from deuterium, halogen, OH, ═O, cyano, C₁₋₄ alkyl or C₁₋₄ alkoxy;
in some embodiments, R^{2a} is selected from deuterium, F, Cl, Br, I, OH, cyano, methyl, ethyl, methoxy or ethoxy;
in some embodiments, R^{2a} and R^{2a} are directly linked to form C₄₋₇ carbocyclic ring or 4- to 7-membered heterocyclic ring, wherein the carbocyclic ring or heterocyclic ring is optionally substituted with 1 to 4 substituents selected from deuterium, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano;
in some embodiments, R^{2a} and R^{2a} are directly linked to form C₄₋₇ carbocyclic ring or 4- to 7-membered heterocyclic ring, wherein the carbocyclic ring or heterocyclic ring is optionally substituted with 1 to 4 substituents selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano;
in some embodiments, R^{2a} and R^{2a} are directly linked to form 4-membered carbocyclic ring, 5-membered carbocyclic ring, 6-membered carbocyclic ring, 4-membered heterocyclic ring, 5-membered heterocyclic ring or 6-membered heterocyclic ring, wherein the carbocyclic ring or heterocyclic ring is optionally substituted with 1 to 4 substituents selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano (such as F, Cl, Br, I, OH or cyano);
in some embodiments, R³ is selected from H or C₁₋₆ alkyl, wherein the alkyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ carbocyclic ring;
in some embodiments, R³ is selected from H or C₁₋₄ alkyl, wherein the alkyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy or C₃₋₆ carbocyclic ring;
in some embodiments, R³ is selected from H or C₁₋₄ alkyl, wherein the alkyl is optionally substituted with 0 to 4 substituents selected from deuterium, halogen, C₁₋₄ alkyl, C₁₋₄ alkoxy or benzene ring;
in some embodiments, R³ is selected from H, methyl, ethyl, propyl, butyl, isobutyl, sec-butyl, tert-butyl or benzyl;
in some embodiments, ring A is selected from C₈₋₁₀ fused carbocyclic ring (such as benzo C₄₋₆ carbocyclic ring), and the ring A is optionally substituted with 0 to 4 R^{a5}; in some embodiments, ring A is selected from

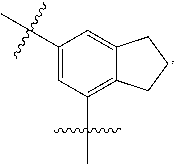 [SMILES: *c1cc(*)c2c(c1)CCC2]

and the ring A is optionally substituted with 0 to 4 R^{a5}; in some embodiments, ring A is selected from

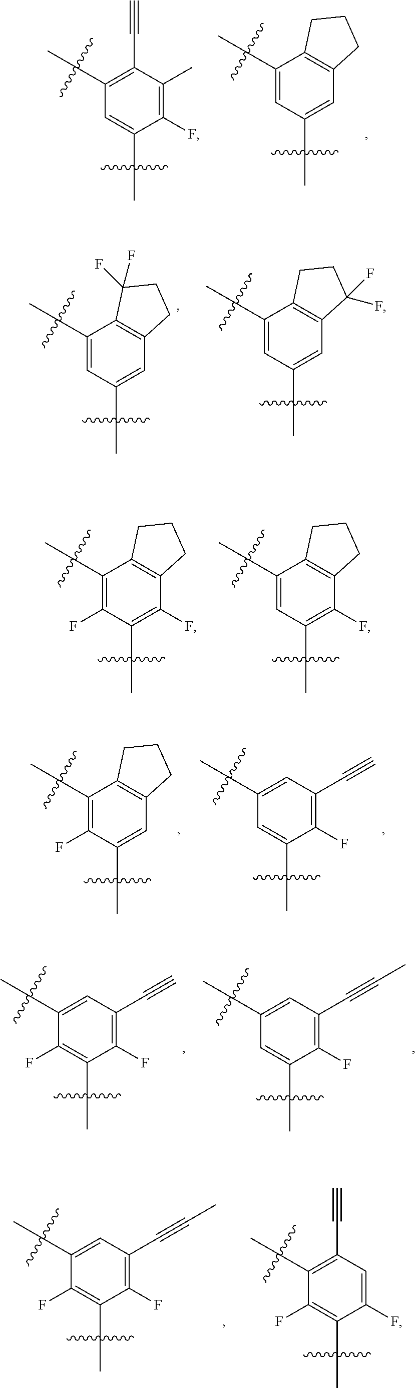

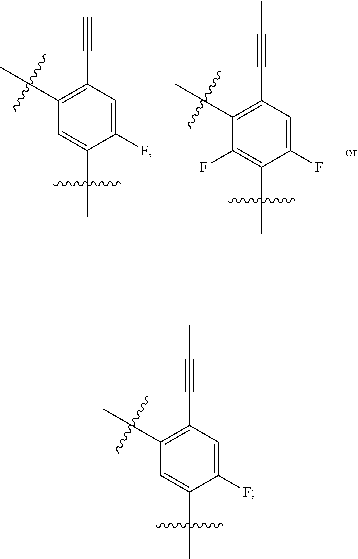 [SMILES: *c1cc(*)c(C#C)cc1F ; *c1c(F)cc(C#CC)c(*)c1F ; *c1cc(*)c(C#CC)cc1F]

in some embodiments, ring A is selected from

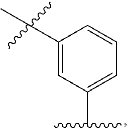 [SMILES: *c1cccc(*)c1]

and the ring A is substituted with one substituent selected from C₂₋₆ alkenyl or C₂₋₆ alkynyl and optionally substituted with 1 to 3 substituents selected from R^{a5}; in some embodiments, ring A is selected from

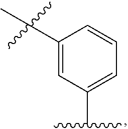 [SMILES: *c1cccc(*)c1]

and the ring A is substituted with one R^{ak} and optionally substituted with 1 to 3 substituents selected from R^{a5}; in some embodiments, ring A is selected from

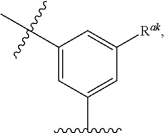

and the ring A is optionally substituted with 1 to 3 substituents selected from R^{a5};
in some embodiments, R^{ak} is selected from C₂₋₄ alkynyl (such as ethynyl, propynyl or propargyl);
in some embodiments, ring A is selected from

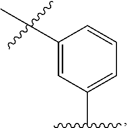 [SMILES: *c1cccc(*)c1]

and the ring A is substituted with one substituent selected from ethenyl, ethynyl, propynyl or propargyl and optionally substituted with 1 to 3 substituents selected from R^{a5};
in some embodiments, R^{a5} is selected from halogen, OH, cyano, C₁₋₆ alkyl, C₂₋₆ alkenyl, C₂₋₆ alkynyl, C₁₋₆ alkoxy, C₃₋₆ cycloalkyl, or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl, and the heterocyclyl contains 1 to 3 heteroatoms selected from N, O or S;
in some embodiments, R^{a5} is selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, and the heterocyclyl contains 1 to 3 heteroatoms selected from N, O or S;
in some embodiments, R^{a5} is selected from halogen, OH, cyano, C₁₋₄ alkyl, C₂₋₄ alkynyl, C₁₋₄ alkoxy or C₃₋₆ cycloalkyl, wherein the alkyl, alkynyl, alkoxy, cycloalkyl or heterocyclyl is optionally substituted with 0 to 4 substituents selected from deuterium, halogen, OH, ═O, cyano, C₁₋₄ alkyl, C₁₋₄ alkoxy or C₃₋₆ cycloalkyl;
in some embodiments, each R⁵ is independently selected from F, Cl, Br, I, OH, CN, ethynyl, propynyl, propargyl, methyl, ethyl, cyclopropyl, methoxy or ethoxy, wherein the ethynyl, propynyl, propargyl, methyl, ethyl, cyclopropyl, methoxy or ethoxy is optionally substituted with 1 to 4 substituents selected from deuterium, halogen, OH, ═O, cyano, C₁₋₄ alkyl, C₁₋₄ alkoxy or C₃₋₆ cycloalkyl;
in some embodiments, each R^{a5} is independently selected from F, Cl, Br, I, OH, CN, ethynyl, methyl, or ethyl;
in some embodiments, each R^{a5} is independently selected from H, halogen, OH, ═O, cyano, COOH, NH₂, NHC₁₋₆ alkyl, N(C₁₋₆ alkyl)₂, C₁₋₆ alkyl, halogen-substituted C₁₋₆ alkyl, cyano-substituted C₁₋₆ alkyl, C₁₋₆ alkoxy or C₃₋₆ cycloalkyl;
in some embodiments, each R^{a5} is independently selected from H, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl;
in some embodiments, each R^{a5} is independently selected from F, Cl, Br, OH, cyano, CF₃, methyl, ethyl, methoxy, ethoxy, cyclopropyl or cyclobutyl;
in some embodiments, ring A is selected from

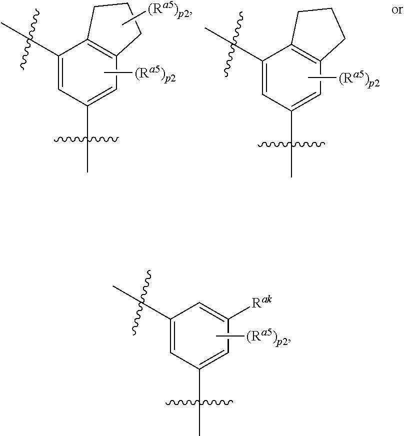

the upper left part of which is directly connected to R², wherein p2 is selected from 0, 1 or 2;
in some embodiments, R^{1b} is selected from

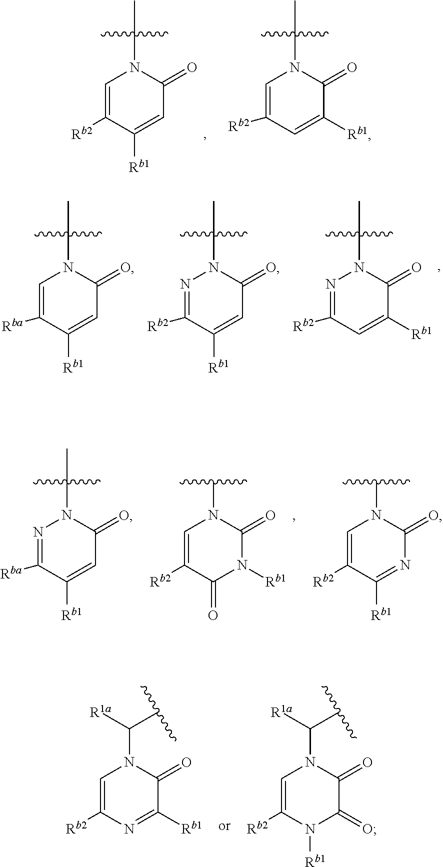

in some embodiments, R^{1b} is selected from

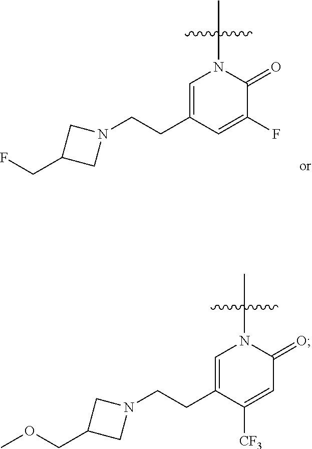 [SMILES: *n1cc(CCN2CC(CF)C2)cc(F)c1=O ; *n1cc(CCN2CC(COC)C2)c(C(F)(F)F)cc1=O]

in some embodiments, each R^{b1} is independently selected from H, halogen, CN, C₁₋₆ alkyl, or C₃₋₆ cycloalkyl, wherein the alkyl or cycloalkyl is optionally substituted with 0 to 4 substituents selected from deuterium, halogen, OH, CN, NHC₁₋₆ alkyl, N(C₁₋₆ alkyl)₂, C₁₋₄ alkyl or C₁₋₆ alkoxy;
in some embodiments, each R^{b1} is independently selected from H, halogen, CN, C₁₋₄ alkyl, or C₃₋₆ cycloalkyl, wherein the alkyl or cycloalkyl is optionally substituted with 0 to 4 substituents selected from deuterium, halogen, OH, CN, NHC₁₋₄ alkyl, N(C₁₋₄ alkyl)₂,
in some embodiments, the compound is not the structure shown in Table E-2 or the stereoisomer thereof;
in some embodiments, the compound of general formula (I) is selected from a compound of general formula (I-a), (I-b), (I-a-1) or (I-b-1),

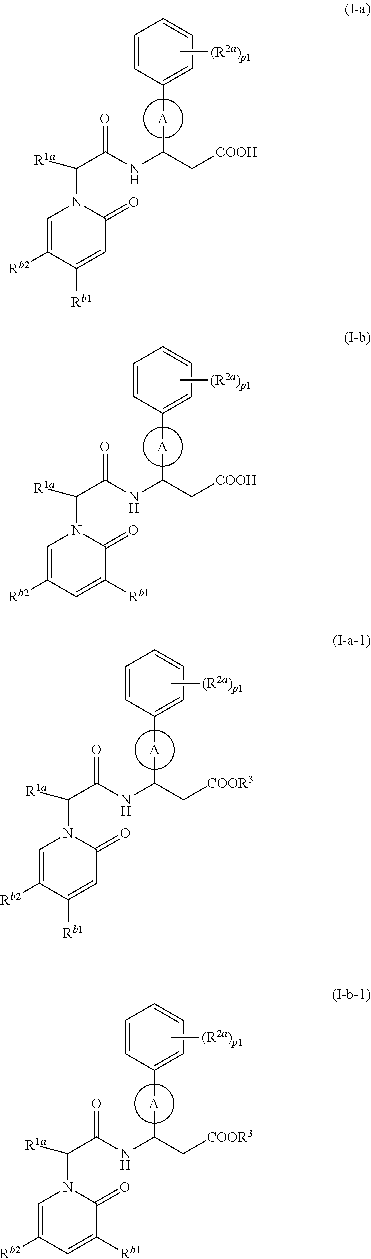

in some embodiments, the compound of general formula (I) is selected from a compound of general formula (I-f), (I-g), (I-f-1), (I-g-1), (I-h), (I-i), (I-h-1) or (I-i-1),

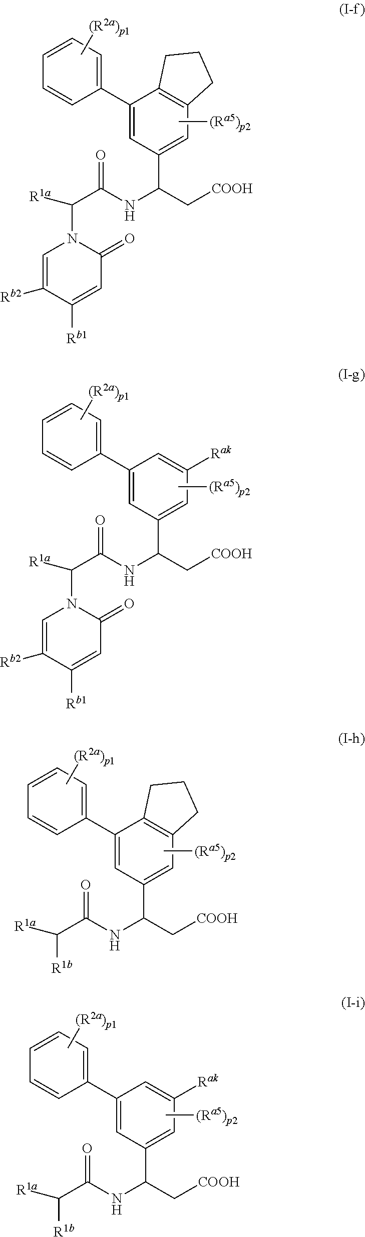

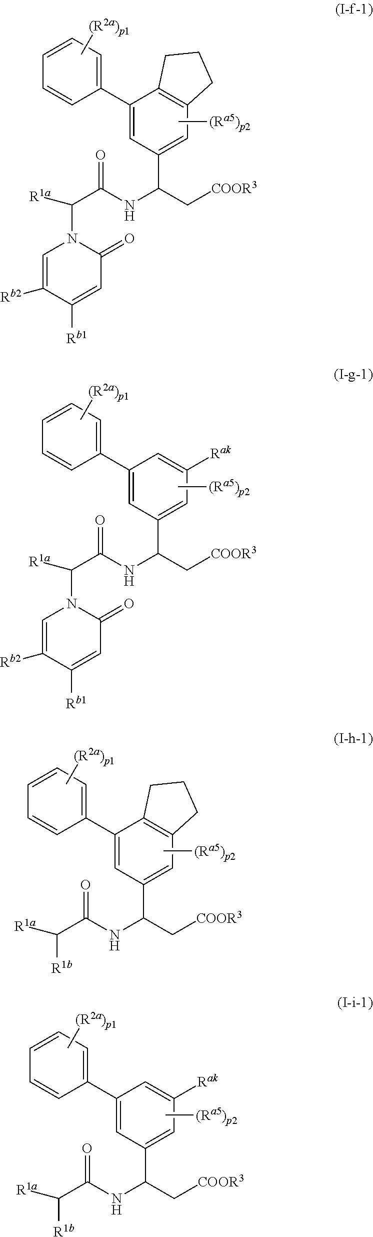

in some embodiments, in general formula (I-a), (I-b), (I-a-1), (I-b-1), (I-f), (I-g), (I-f-1), (I-g-1), (I-h), (I-i), (I-h-1) or (I-i-1), the definitions of R^{2a}, R^{1a}, R^{ak}, R^{a5}, R^{b2}, R^{b1} and R³ are the same as those in any one of the preceding embodiments;
in some embodiments, in general formula (I-a) or (I-b), the definition of ring A is the same as that in any one of the preceding embodiments of ring A;
in some embodiments, in general formula (I), (I-a), (I-b), (I-a-1), (I-b-1), (I-f), (I-g), (I-f-1), (I-g-1), (I-h), (I-i), (I-h-1) or (I-i-1), p1 is selected from 0, 1, 2, 3 or 4;
in some embodiments, in general formula (I), (I-a), (I-b), (I-a-1), (I-b-1), (I-f), (I-g), (I-f-1), (I-g-1), (I-h), (I-i), (I-h-1) or (I-i-1), R^{2a} is selected from deuterium, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from deuterium, halogen, OH, CN, C₁₋₄ alkyl, or C₁₋₄ alkoxy, and the heterocycloalkyl contains 1 to 4 heteroatoms selected from O, S or N;
in some embodiments, in general formula (I), (I-a), (I-b), (I-a-1), (I-b-1), (I-f), (I-g), (I-f-1), (I-g-1), (I-h), (I-i), (I-h-1) or (I-i-1), R^{1a} is selected from

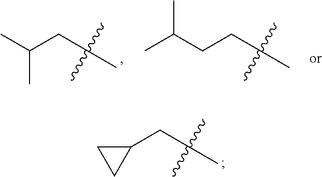

in some embodiments, in general formula (I), (I-a), (I-b), (I-a-1), (I-b-1), (I-f), (I-g), (I-f-1), (I-g-1), (I-h), (I-i), (I-h-1) or (I-i-1), R^{b1} is selected from H, C₁₋₄ alkyl or C₃₋₆ cycloalkyl, wherein the alkyl or cycloalkyl is optionally substituted with 0 to 4 substituents selected from deuterium, halogen, OH, CN, NHC₁₋₄ alkyl, N(C₁₋₄ alkyl)₂, C₁₋₄ alkyl or C₁₋₄ alkoxy;
in some embodiments, in general formula (I), (I-a), (I-b), (I-f), (I-g), (I-f-1), (I-g-1), (I-h), (I-i), (I-h-1) or (I-i-1), R^{b1} is selected from H, F, CH₂F, CHF₂, CF₃, methyl or —CH₂CH₂N(CH₃)₂;
in some embodiments, in general formula (I), (I-a), (I-b), (I-f), (I-g), (I-f-1), (I-g-1), (I-h), (I-i), (I-h-1) or (I-i-1), R^{b2} is selected from H, R^{ba}, or one of the following substituted or unsubstituted groups: C₁₋₄ alkyl, C₂₋₄ alkynyl, C₁₋₄ alkoxy, —CH₂CH₂—NHC₁₋₄ alkyl, —CH₂CH₂—N(C₁₋₄ alkyl)₂, —CH₂CH₂—C₃₋₆ cycloalkyl, —CH₂CH₂-3- to 7-membered heterocycloalkyl, wherein the CH₂, alkyl, cycloalkyl or heterocycloalkyl is optionally substituted with 0 to 4 substituents selected from deuterium, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
in some embodiments, in general formula (I), (I-a), (I-b), (I-f), (I-g), (I-f-1), (I-g-1), (I-h), (I-i), (I-h-1) or (I-i-1), R^{b2} is selected from H, R^{ba}, or one of the following substituted or unsubstituted groups: methyl, ethyl, ethynyl, methoxy, ethoxy, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl or —CH₂CH₂-morpholine, which, when substituted, is substituted with 1, 2 or 3 substituents selected from deuterium, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
in some embodiments, in general formula (I-a), (I-b), (I-f), (I-g), (I-f-1), (I-g-1), (I-h), (I-i), (I-h-1) or (I-i-1), the definitions of R^{ba} and R^{k} are the same as those in any one of the preceding embodiments;
in some embodiments, in general formula (I), (I-a), (I-b), (I-f), (I-g), (I-f-1), (I-g-1), (I-h), (I-i), (I-h-1) or (I-i-1), R^{2a} is selected from deuterium, —CD₃, —OCD₃, F, Cl, Br, I, OH, cyano, methyl, ethyl, methoxy or ethoxy;
in some embodiments, in general formula (I), (I-f), (I-g), (I-f-1), (I-g-1), (I-h), (I-i), (I-h-1) or (I-i-1), p2 is selected from 0, 1 or 2;
in some embodiments, in general formula (I), (I-g), (I-g-1), (I-i) or (I-i-1), R^{ak} is selected from C₂₋₄ alkynyl;
in some embodiments, in general formula (I), (I-f), (I-g), (I-f-1), (I-g-1), (I-h), (I-i), (I-h-1) or (I-i-1), R^{a5} is selected from F, Cl, Br, I, OH, cyano, methyl, ethyl, methoxy, ethoxy or ethynyl;
in some embodiments, in general formula (I), n1 is selected from 1, 2 or 3.

As a first embodiment of the present disclosure, provided is the compound of preceding general formula (I) or a stereoisomer, mesomer/racemate, deuterated product, solvate, prodrug, metabolite, pharmaceutically acceptable salt, or co-crystal thereof, wherein
R¹ is selected from —CHR^{1a}R^{1b} or —NR^{1a}R^{1b};
R^{1a} is selected from C₁₋₆ alkyl, wherein the alkyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl;
R^{1b} is selected from C₄₋₁₀ carbocyclic ring or 5- to 10-membered heterocyclic ring, wherein the carbocyclic ring or heterocyclic ring is optionally substituted with 0 to 4 R^{b}, and the heterocyclic ring contains 1 to 4 heteroatoms selected from O, S or N;
each R^{b} is independently selected from deuterium, halogen, OH, ═O, cyano, COOH, NH₂, —C₀₋₄ alkyl-NHC₁₋₆ alkyl, —C₀₋₄ alkyl-N(C₁₋₆ alkyl)₂, C₁₋₆ alkyl, C₂₋₆ alkynyl, C₁₋₆ alkoxy, —C₀₋₄ alkyl-C₃₋₁₀ carbocyclic ring, —C₀₋₄ alkyl-3- to 10-membered heterocyclic ring or R^{ba}, wherein the alkyl, alkynyl, alkoxy, carbocyclic ring or heterocyclic ring is optionally substituted with 0 to 4 substituents selected from deuterium, halogen, OH, ═O, cyano, COOH, CONH₂, CONHC₁₋₆ alkyl, CON(C₁₋₆ alkyl)₂, NH₂, NHC₁₋₆ alkyl, N(C₁₋₆ alkyl)₂, N(C₁₋₆ alkyl)(C₃₋₆ cycloalkyl), NH(C₃₋₆ cycloalkyl), C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, and the heterocyclic ring contains 1 to 4 heteroatoms selected from O, S or N;
R^{ba} is selected from —C₀₋₄ alkyl-7- to 12-membered heterocyclic ring, —C₀₋₄ alkyl-4- to 6-membered heterocyclic ring connected via a carbon atom,

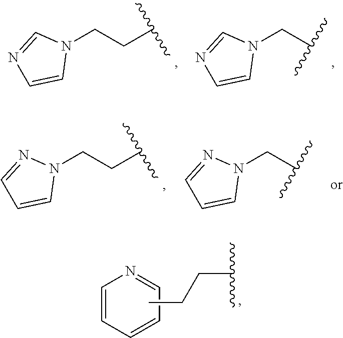

and the R^{ba} is optionally substituted with 1 to 4 substituents selected from H, halogen, OH, ═O, cyano, COOH, CONH₂, CONHC₁₋₆ alkyl, CON(C₁₋₆ alkyl)₂, NH₂, NHC₁₋₆ alkyl, N(C₁₋₆ alkyl)₂, C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, wherein the heterocyclic ring contains 1 to 4 heteroatoms selected from O, S or N; R^{k} is selected from —C₁₋₄ alkyl-NH₂, —C₁₋₄ alkyl-NHC₁₋₆ alkyl, —C₁₋₄ alkyl-N(C₁₋₆ alkyl)₂, —C₀₋₄ alkyl-C₃₋₁₀ carbocyclic ring or —C₀₋₄ alkyl-3- to 10-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy, halogen-substituted C₁₋₆ alkyl, halogen-substituted C₁₋₆ alkoxy or C₁₋₆ alkoxyalkyl, and the heterocyclic ring contains 1 to 4 heteroatoms selected from O, S or N;
R² is selected from C₁₋₆ alkyl, C₆₋₁₀ aromatic ring, 5- to 10-membered heteroaromatic ring, C₃₋₁₀ carbocyclic ring or 5- to 10-membered heterocyclic ring, and the R² is optionally substituted with 0 to 4 R^{2a}, wherein the heteroaromatic ring or heterocyclic ring contains 1 to 4 heteroatoms selected from O, S or N;
each R^{2a} is independently selected from deuterium, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
as an option, R^{2a} and R^{2a} are directly linked to form C₄₋₇ carbocyclic ring or 4- to 7-membered heterocyclic ring, wherein the carbocyclic ring or heterocyclic ring is optionally substituted with 1 to 4 substituents selected from deuterium, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano;
R³ is selected from H or C₁₋₆ alkyl, wherein the alkyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ carbocyclic ring;
ring A is selected from C₈₋₁₀ fused carbocyclic ring, and the ring A is optionally substituted with 0 to 4 R^{a5};
or ring A is selected from

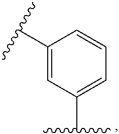 [SMILES: *c1cccc(*)c1]

and the ring A is substituted with one substituent selected from C₂₋₆ alkenyl or C₂₋₆ alkynyl and optionally substituted with 1 to 3 R^{a5};
R^{a5} is selected from halogen, OH, cyano, C₁₋₆ alkyl, C₂₋₆ alkenyl, C₂₋₆ alkynyl, C₁₋₆ alkoxy, C₃₋₆ cycloalkyl, or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl, and the heterocyclyl contains 1 to 3 heteroatoms selected from N, O or S.

As a second embodiment of the present disclosure, provided is the compound of preceding general formula (I) or a stereoisomer, mesomer/racemate, deuterated product, solvate, prodrug, metabolite, pharmaceutically acceptable salt, or co-crystal thereof, wherein
R¹ is selected from

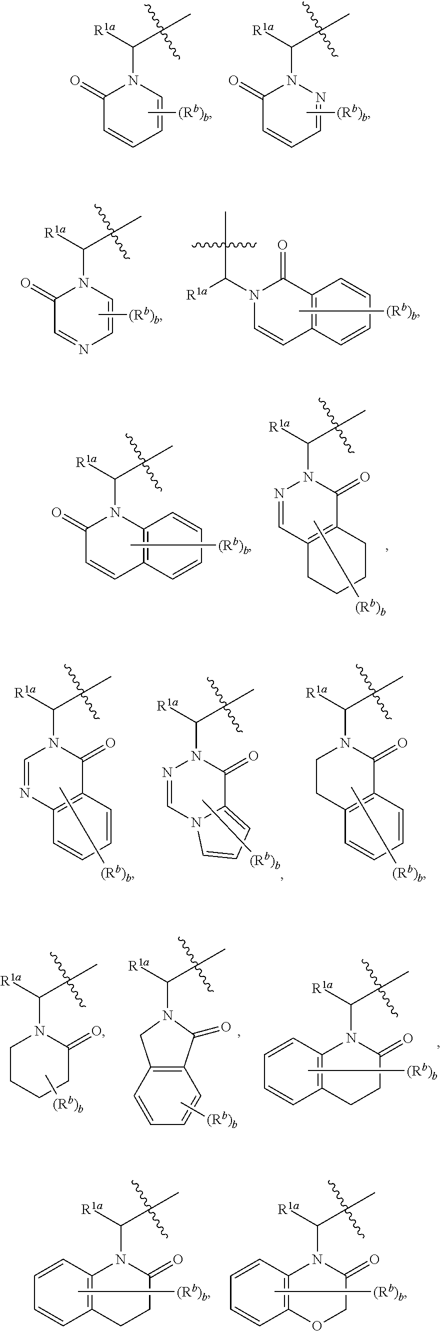

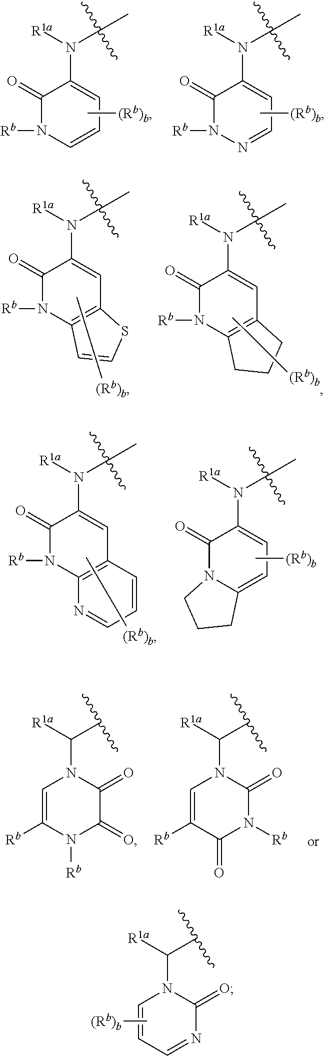

b is selected from 0, 1, 2 or 3;
each R^{b} is independently selected from deuterium, halogen, OH, ═O, cyano, COOH, NH₂, —C₀₋₄ alkyl-NHC₁₋₄ alkyl, —C₀₋₄ alkyl-N(C₁₋₄ alkyl)₂, C₁₋₄ alkyl, C₂₋₄ alkynyl, C₁₋₄ alkoxy, —C₀₋₄ alkyl-C₃₋₆ carbocyclic ring, —C₀₋₄ alkyl-3- to 7-membered heterocyclic ring or R^{ba}, wherein the alkyl, alkynyl, alkoxy, carbocyclic ring or heterocyclic ring is optionally substituted with 0 to 4 substituents selected from deuterium, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heterocyclic ring contains 1 to 4 heteroatoms selected from O, S or N;
R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl connected via a carbon atom,

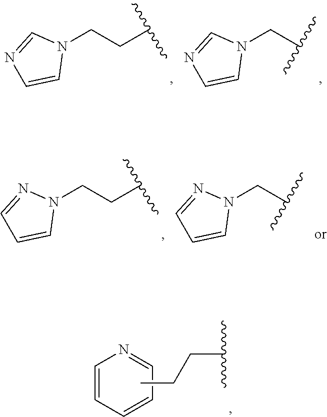

and the R^{ba} is optionally substituted with 1 to 4 substituents selected from H, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclic ring or —C₀₋₂ alkyl-3- to 6-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclic ring contains 1 to 4 heteroatoms selected from O, S or N;
R² is selected from C₁₋₄ alkyl, benzene ring, naphthalene ring, 5- to 6-membered heteroaromatic ring, 9- to 10-membered heteroaromatic ring, C₃₋₁₀ non-aromatic carbocyclic ring, 5- to 10-membered non-aromatic heterocyclic ring, benzo C₄₋₆ carbocyclyl or benzo 4- to 6-membered heterocyclyl, and the R² is optionally substituted with 0 to 4 R^{2a}, wherein the heteroaromatic ring or heterocyclic ring contains 1 to 4 heteroatoms selected from O, S or N;
each R^{2a} is independently selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
as an option, R^{2a} and R^{2a} are directly linked to form C₄₋₇ carbocyclic ring or 4- to 7-membered heterocyclic ring, wherein the carbocyclic ring or heterocyclic ring is optionally substituted with 1 to 4 substituents selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano;
R³ is selected from H or C₁₋₄ alkyl, wherein the alkyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy or C₃₋₆ carbocyclic ring;
ring A is selected from benzo C₄₋₆ carbocyclic ring, and the ring A is optionally substituted with 0 to 4 R^{a5};
or ring A is selected from

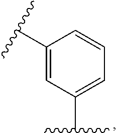 [SMILES: *c1cccc(*)c1]

and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, and the heterocyclyl contains 1 to 3 heteroatoms selected from N, O or S;
the definitions of the remaining substituents are consistent with those in the first embodiment of the present disclosure.

As a third embodiment of the present disclosure, provided is the compound of preceding general formula (I) or a stereoisomer, mesomer/racemate, deuterated product, solvate, prodrug, metabolite, pharmaceutically acceptable salt, or co-crystal thereof, wherein
each R^{b} is independently selected from deuterium, halogen, OH, ═O, cyano, C₁₋₄ alkyl, C₂₋₄ alkynyl, C₁₋₄ alkoxy, phenyl, 5- to 6-membered heteroaryl, —CH₂NHC₁₋₄ alkyl, —CH₂N(C₁₋₄ alkyl)₂, —CH₂CH₂—NHC₁₋₄ alkyl, —CH₂CH₂—N(C₁₋₄ alkyl)₂, phenyl, 5- to 6-membered heteroaryl, C₃₋₆ cycloalkyl, 3- to 7-membered heterocycloalkyl, —CH₂-phenyl, —CH₂-5- to 6-membered heteroaryl, —CH₂—C₃₋₆ cycloalkyl, —CH₂-3- to 7-membered heterocycloalkyl, —CH₂CH₂-phenyl, —CH₂CH₂-5- to 6-membered heteroaryl, —CH₂CH₂—C₃₋₆ cycloalkyl, —CH₂CH₂-3- to 7-membered heterocycloalkyl or R^{ba}, wherein the CH₂, alkyl, alkynyl, phenyl, heteroaryl, cycloalkyl or heterocycloalkyl is optionally substituted with 0 to 4 substituents selected from deuterium, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N; R^{ba} is selected from 7- to 8-membered monocyclic heterocycloalkyl, 7- to 11-membered spiro heterocycloalkyl, 7- to 11-membered bridged heterocycloalkyl, 4- to 6-membered monocyclic heterocycloalkyl connected via a carbon atom, —CH₂-7- to 8-membered monocyclic heterocycloalkyl, —CH₂-7- to 11-membered spiro heterocycloalkyl, —CH₂-7- to 11-membered bridged heterocycloalkyl, —CH₂-4- to 6-membered monocyclic heterocycloalkyl connected via a carbon atom, —CH₂CH₂-7- to 8-membered monocyclic heterocycloalkyl, —CH₂CH₂-7- to 11-membered spiro heterocycloalkyl, —CH₂CH₂-7- to 11-membered bridged heterocycloalkyl, —CH₂CH₂-4- to 6-membered monocyclic heterocycloalkyl connected via a carbon atom,

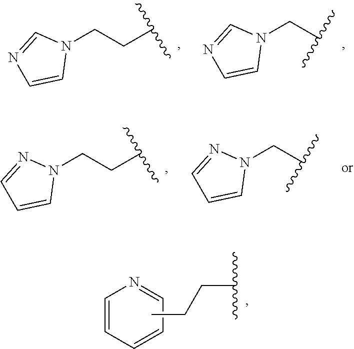

and the R^{ba} is optionally substituted with 1 to 4 substituents selected from H, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₄ alkyl-NH₂, —C₁₋₄ alkyl-NHC₁₋₄ alkyl, —C₁₋₄ alkyl-N(C₁₋₄ alkyl)₂, —C₀₋₄ alkyl-C₃₋₆ carbocyclic ring or —C₀₋₄ alkyl-3- to 6-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclic ring contains 1 to 4 heteroatoms selected from O, S or N;
R² is selected from benzene ring, naphthalene ring, 5- to 6-membered heteroaromatic ring, 9- to 10-membered heteroaromatic ring, C₃₋₆ cycloalkyl, 3- to 7-membered heterocycloalkyl, benzo C₄₋₆ carbocyclyl or benzo 4- to 6-membered heterocyclyl, and the R² is optionally substituted with 0 to 4 R^{2a}, wherein the heteroaromatic ring or heterocycloalkyl contains 1 to 4 heteroatoms selected from O, S or N;
each R^{2a} is independently selected from deuterium, halogen, OH, cyano, ═O, C₁₋₄ alkyl, C₂₋₄ alkenyl, C₂₋₄ alkynyl, C₁₋₄ alkoxy or C₃₋₆ cycloalkyl, wherein the alkyl, alkenyl, alkynyl, alkoxy or cycloalkyl is optionally substituted with 0 to 4 substituents selected from deuterium, halogen, OH, ═O, cyano, C₁₋₄ alkyl or C₁₋₄ alkoxy;
as an option, R^{2a} and R^{2a} are directly linked to form 4-membered carbocyclic ring, 5-membered carbocyclic ring, 6-membered carbocyclic ring, 4-membered heterocyclic ring, 5-membered heterocyclic ring or 6-membered heterocyclic ring, wherein the carbocyclic ring or heterocyclic ring is optionally substituted with 1 to 4 substituents selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano;
R³ is selected from H or C₁₋₄ alkyl, wherein the alkyl is optionally substituted with 0 to 4 substituents selected from deuterium, halogen, C₁₋₄ alkyl, C₁₋₄ alkoxy or benzene ring;
ring A is selected from

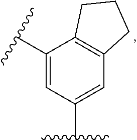 [SMILES: *c1cc(*)c2c(c1)CCC2]

and the ring A is optionally substituted with 0 to 4 R^{a5};
or ring A is selected from

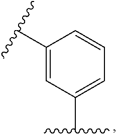 [SMILES: *c1cccc(*)c1]

and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkynyl, C₁₋₄ alkoxy or C₃₋₆ cycloalkyl, wherein the alkyl, alkynyl, alkoxy, cycloalkyl or heterocyclyl is optionally substituted with 0 to 4 substituents selected from deuterium, halogen, OH, ═O, cyano, C₁₋₄ alkyl, C₁₋₄ alkoxy or C₃₋₆ cycloalkyl;
the definitions of the remaining substituents are the same as those in any one of the first or second embodiment of the present disclosure.

As a forth embodiment of the present disclosure, provided is the compound of preceding general formula (I) or a stereoisomer, mesomer/racemate, deuterated product, solvate, prodrug, metabolite, pharmaceutically acceptable salt, or co-crystal thereof, wherein
R^{1a} is selected from methyl, ethyl, propyl, butyl, isobutyl, sec-butyl, tert-butyl, —CH₂-cyclopropyl or —CH₂-cyclobutyl;
each R^{b} is independently selected from deuterium, F, Cl, Br, I, OH, ═O, cyano or R^{ba}, or
each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azolidinyl, azinanyl, oxetanyl, oxolanyl, oxanyl, morpholinyl, phenyl, pyridine, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azolidinyl, —CH₂-azinanyl, —CH₂-oxetanyl, —CH₂-oxolanyl, —CH₂-oxanyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl, —CH₂CH₂-morpholinyl, —CH₂-phenyl, —CH₂-pyridyl, —CH₂CH₂-phenyl or —CH₂CH₂-pyridyl, which, when substituted, is substituted with 0 to 4 substituents selected from deuterium, halogen, OH, ═O, cyano, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
R^{ba} is selected from one of the following optionally substituted groups:

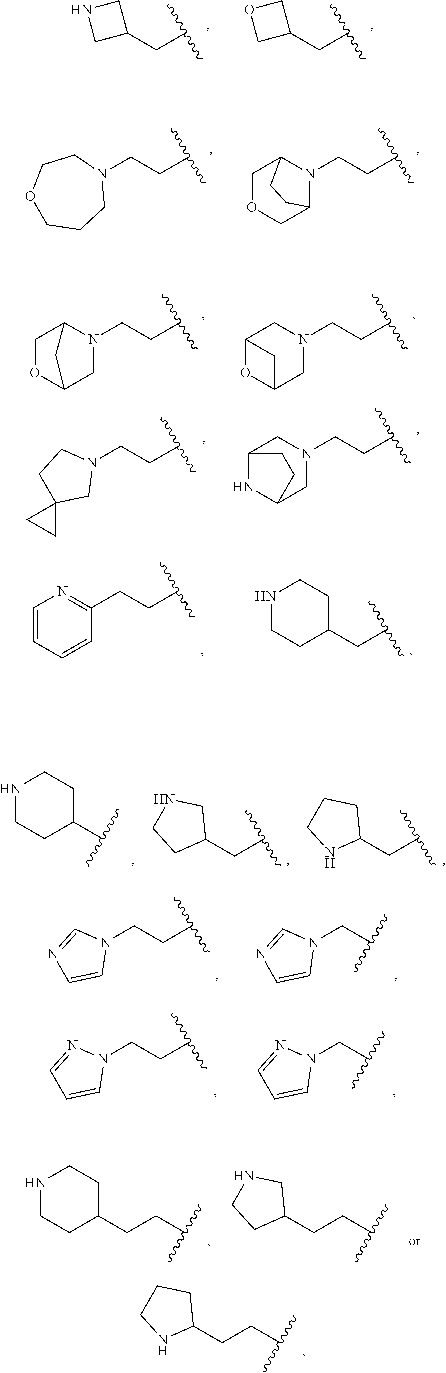

which, when substituted, is optionally substituted with 1 to 4 substituents selected from deuterium, halogen, OH, ═O, cyano, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
R^{k} is selected from —CH₂N(CH₃)₂, —CH₂-cyclopropyl, —CH₂-cyclobutyl, cyclopropyl, cyclobutyl, cyclopentyl, cyclohexyl, azetidinyl, azolidinyl, azinanyl or oxetanyl, wherein the cyclopropyl, cyclobutyl, cyclopentyl, cyclohexyl, azetidinyl, azolidinyl or azinanyl is optionally substituted with 1 to 4 substituents selected from deuterium, halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₁₋₄ alkoxy, halogen-substituted C₁₋₄ alkyl, halogen-substituted C₁₋₄ alkoxy or C₁₋₄ alkoxyalkyl;
R² is selected from benzene ring, pyridyl, pyridonyl, pyrazinyl, pyrimidyl, thienyl, thiazolyl, furyl, oxazolyl, pyrrolyl, pyrazolyl, imidazolyl, cyclopropyl, cyclobutyl, cyclopentyl, cyclohexyl, azetidinyl, azolidinyl, azinanyl, oxetanyl, oxolanyl, oxanyl, morpholinyl,

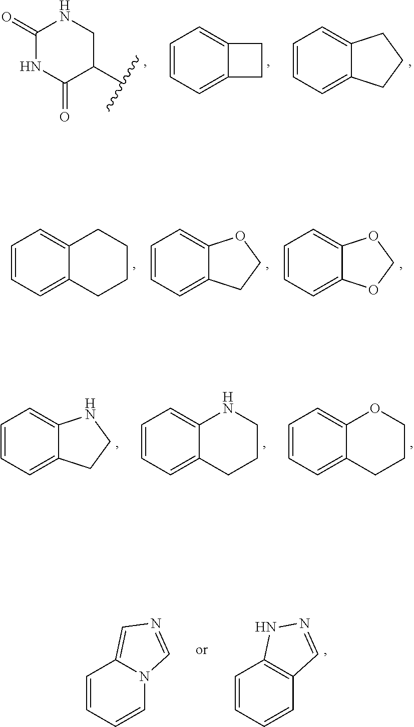

and the R² is optionally substituted with 0 to 4 R^{2a}; each R^{2a} is independently selected from deuterium, F, Cl, Br, I, OH, ═O, cyano, methyl, ethyl, ethenyl, ethynyl, methoxy, ethoxy, cyclopropyl or cyclobutyl, wherein the methyl, ethyl, ethenyl, ethynyl, methoxy, ethoxy, cyclopropyl or cyclobutyl is optionally substituted with 0 to 4 substituents selected from deuterium, halogen, OH, ═O, cyano, C₁₋₄ alkyl or C₁₋₄ alkoxy;
R³ is selected from H, methyl, ethyl, propyl, butyl, isobutyl, sec-butyl, tert-butyl or benzyl;
ring A is selected from

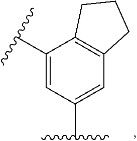 [SMILES: *c1cc(*)c2c(c1)CCC2]

and the ring A is optionally substituted with 0 to 4 R^{a5};
or ring A is selected from

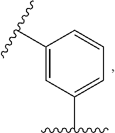 [SMILES: *c1cccc(*)c1]

and the ring A is substituted with one substituent selected from ethenyl, ethynyl, propynyl or propargyl and optionally substituted with 1 to 3 substituents selected from R^{a5};
each R^{a5} is independently selected from F, Cl, Br, I, OH, CN, ethynyl, propynyl, propargyl, methyl, ethyl, cyclopropyl, methoxy or ethoxy, wherein the ethynyl, propynyl, propargyl, methyl, ethyl, cyclopropyl, methoxy or ethoxy is optionally substituted with 1 to 4 substituents selected from deuterium, halogen, OH, ═O, cyano, C₁₋₄ alkyl, C₁₋₄ alkoxy or C₃₋₆ cycloalkyl;
the definitions of the remaining substituents are the same as those in any one of the first, second or third embodiment of the present disclosure.

As a fifth embodiment of the present disclosure, provided is the compound of preceding general formula (I) or a stereoisomer, mesomer/racemate, deuterated product, solvate, prodrug, metabolite, pharmaceutically acceptable salt, or co-crystal thereof, wherein
R¹ is selected from

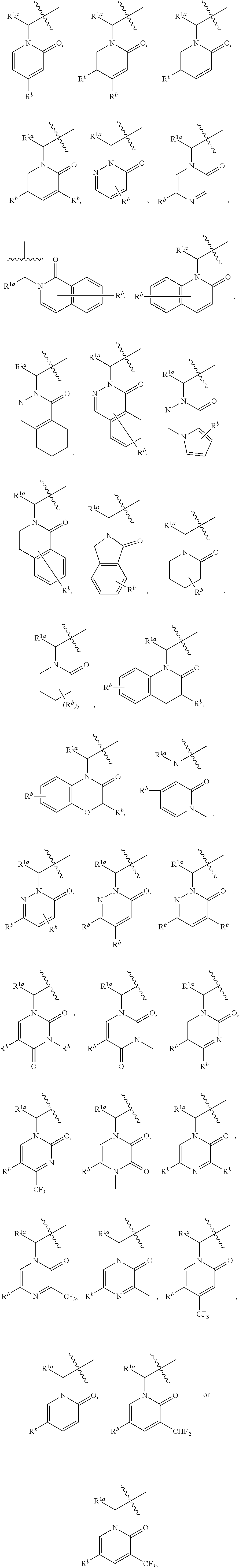

each R^{b} is independently selected from deuterium, F, Cl, Br, OH, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azolidinyl, azinanyl, oxetanyl, oxolanyl, oxanyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azolidinyl, —CH₂-azinanyl, —CH₂-oxetanyl, —CH₂-oxolanyl, —CH₂-oxanyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl, —CH₂CH₂-morpholinyl, —CH₂-phenyl, —CH₂-pyridyl, —CH₂CH₂-phenyl, —CH₂CH₂-pyridyl or

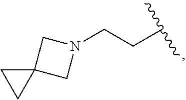 [SMILES: *CCN1CC2(CC2)C1]

which, when substituted, is substituted with 0 to 4 substituents selected from deuterium, F, Cl, Br, OH, ═O, cyano, CONH₂, CONHCH₃, CON(CH₃)₂, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxy methyl, ethoxymethyl, methoxyethyl or R^{k};
R^{ba} is selected from one of the following optionally substituted groups:

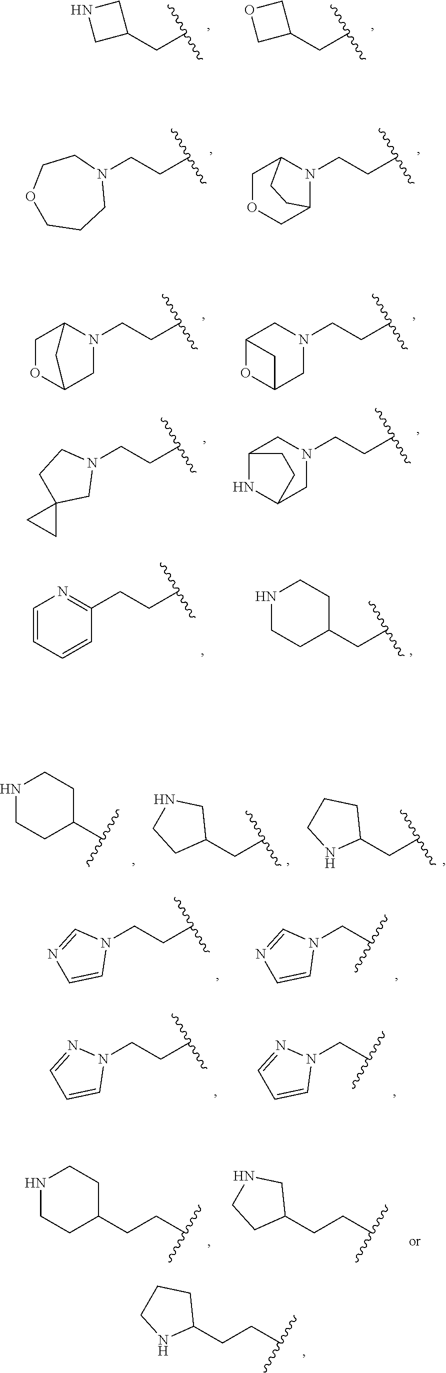

which, when substituted, is optionally substituted with 1 to 4 substituents selected from deuterium, F, Cl, Br, OH, ═O, cyano, CONH₂, CONHCH₃, CON(CH₃)₂, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{k} is selected from —CH₂N(CH₃)₂, —CH₂-cyclopropyl, —CH₂-cyclobutyl, cyclopropyl, cyclobutyl, cyclopentyl, cyclohexyl, azetidinyl, azolidinyl, azinanyl or oxetanyl, wherein the cyclopropyl, cyclobutyl, cyclopentyl, cyclohexyl, azetidinyl, azolidinyl, azinanyl or oxetanyl is optionally substituted with 1 to 4 substituents selected from deuterium, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, methoxy, ethoxy, methoxymethyl, ethoxymethyl or methoxyethyl;
R² is selected from phenyl, pyridyl, pyridonyl, azolidinyl, morpholinyl or

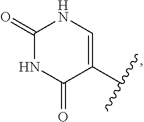 [SMILES: *c1c[nH]c(=O)[nH]c1=O]

and the R² is optionally substituted with 0 to 4 substituents selected from deuterium, F, Cl, Br, OH, CF₃, cyano, methyl, ethyl, methoxy, ethoxy, cyclopropyl or cyclobutyl;
or R² is selected from

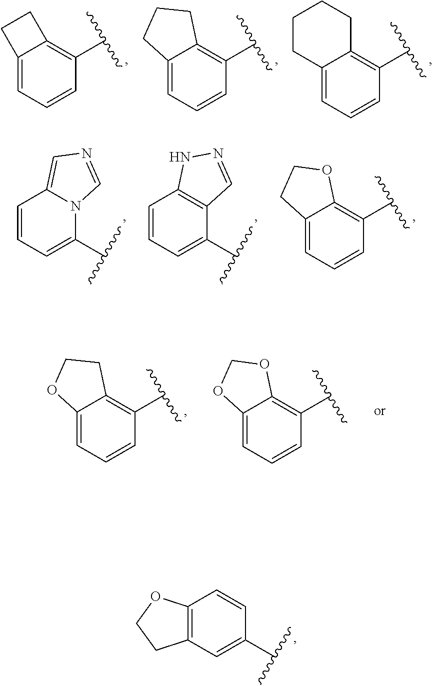

and the R² is optionally substituted with 1 to 4 substituents selected from deuterium, CD₃, —OCD₃, F, Cl, Br, OH, CF₃, cyano, methyl, ethyl, methoxy, ethoxy, cyclopropyl or cyclobutyl;
ring A is selected from

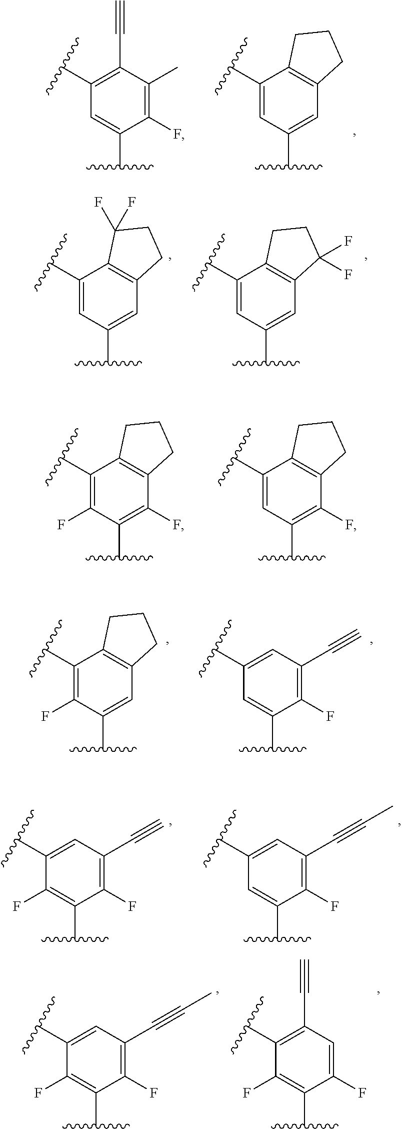

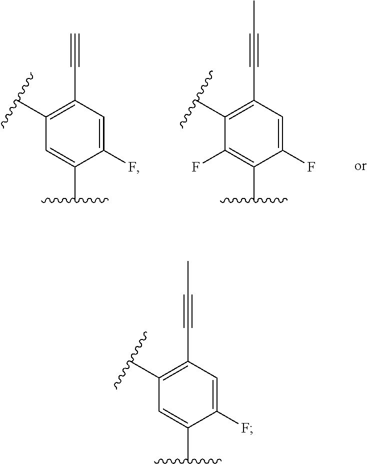 [SMILES: *c1cc(*)c(C#C)cc1F ; *c1c(F)cc(C#CC)c(*)c1F ; "or" ; *c1cc(*)c(C#CC)cc1F]

the definitions of the remaining substituents are the same as those in any one of the first, second, third or fourth embodiment of the present disclosure.

As a sixth embodiment of the present disclosure, provided is the compound of above general formula (I) or a stereoisomer, mesomer/racemate, deuterated product, solvate, prodrug, metabolite, pharmaceutically acceptable salt, or co-crystal thereof, wherein
R¹ is selected from

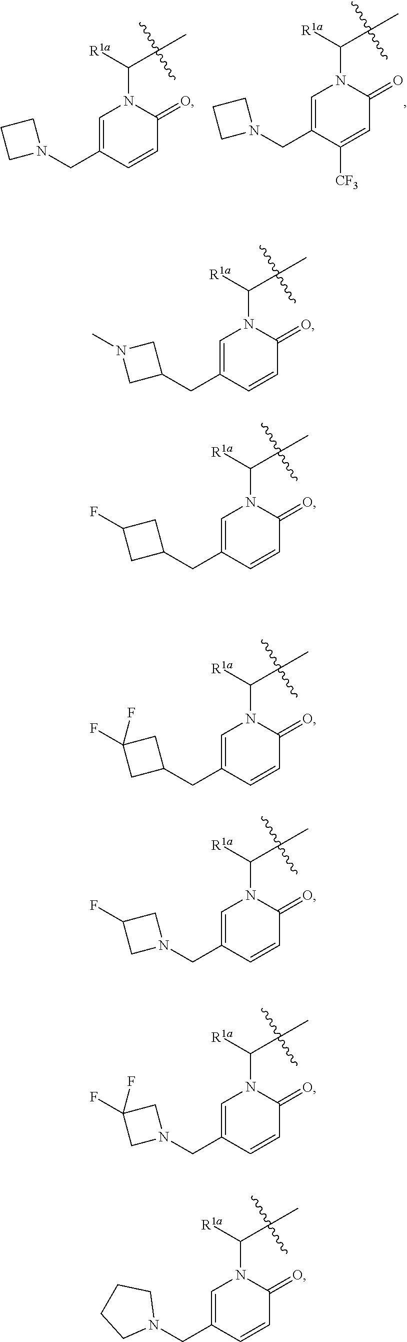

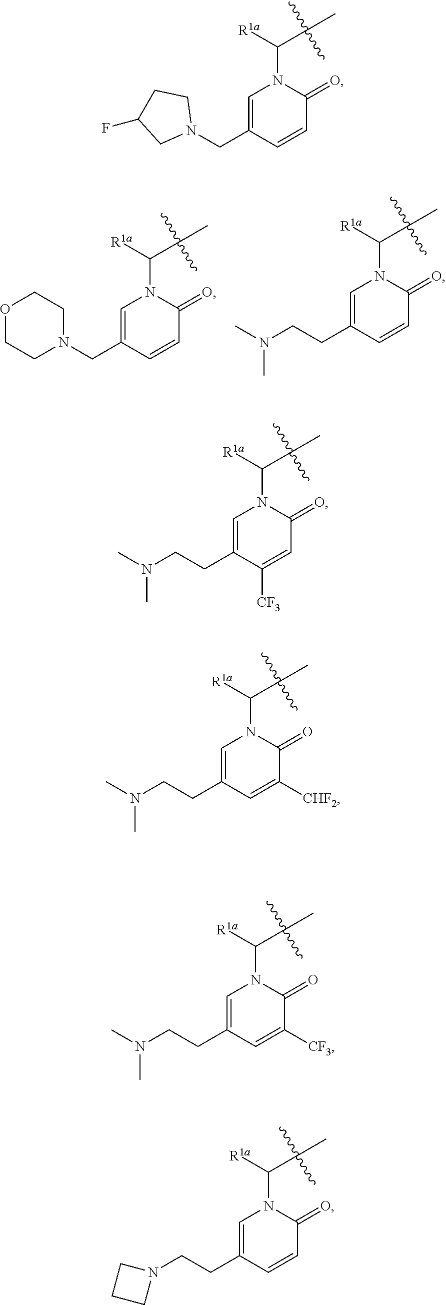

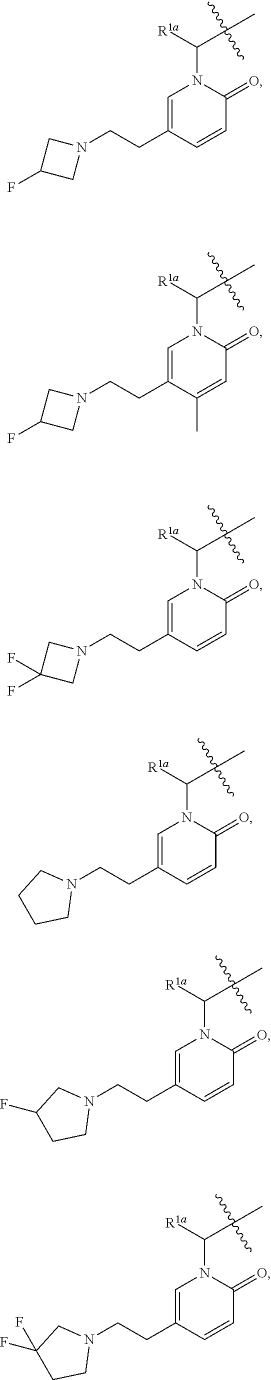

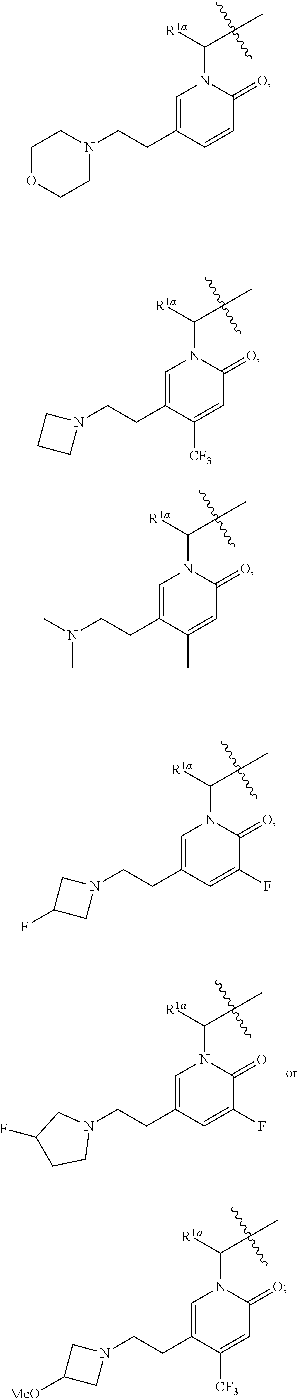

or R¹ is selected from

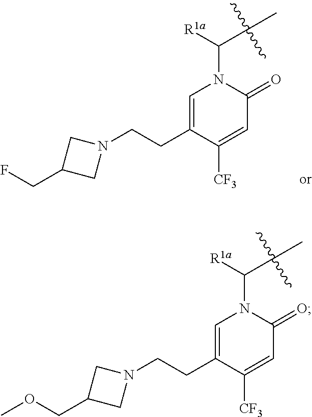

or R¹ is selected from

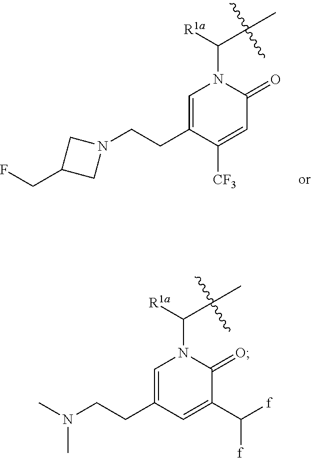

or R¹ is selected from

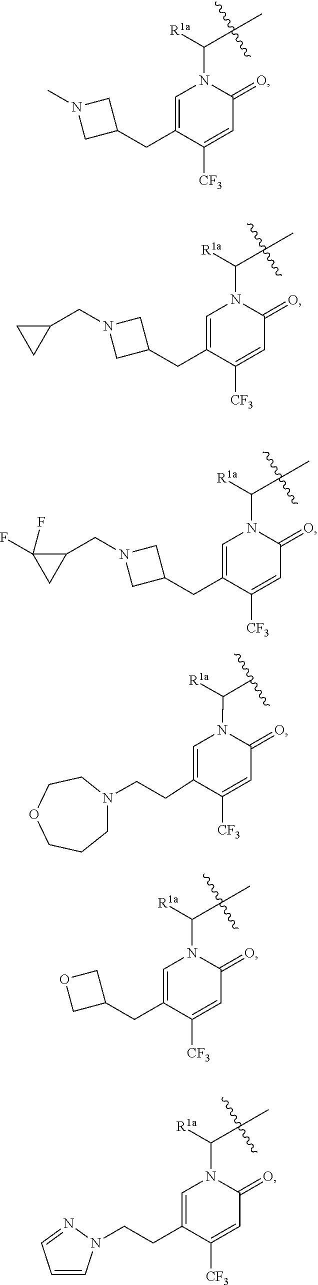

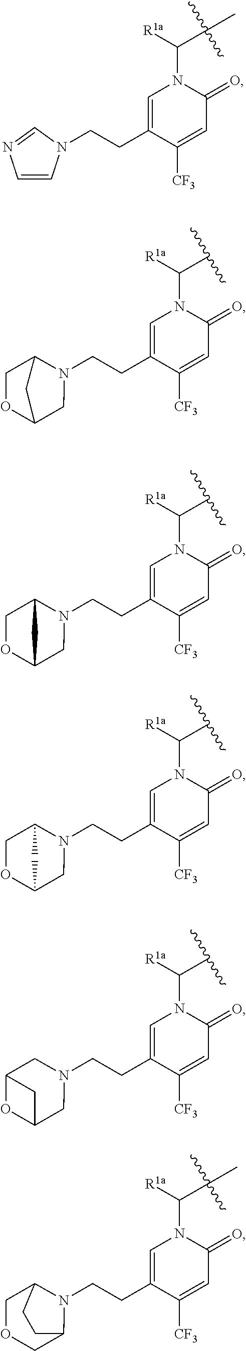

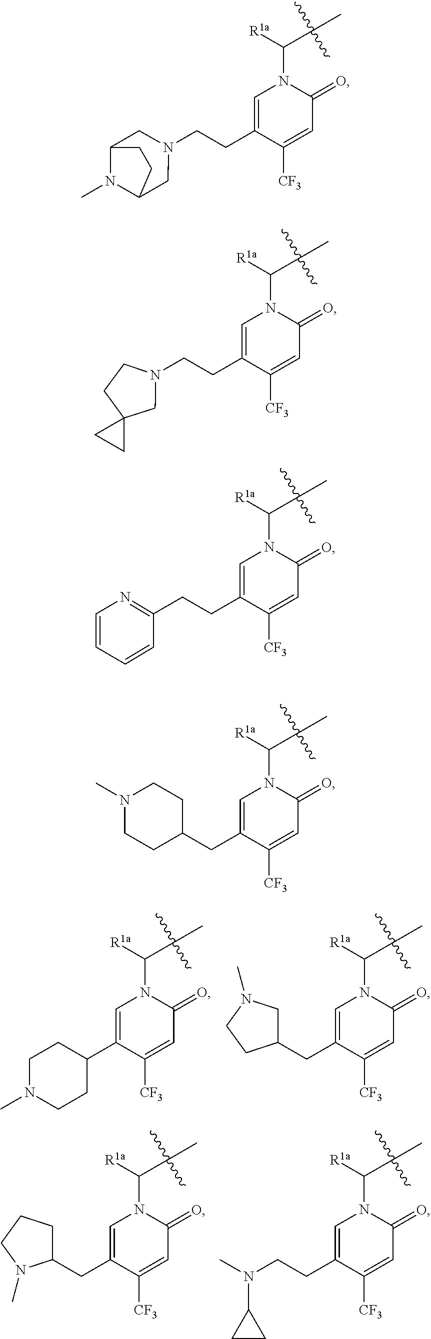

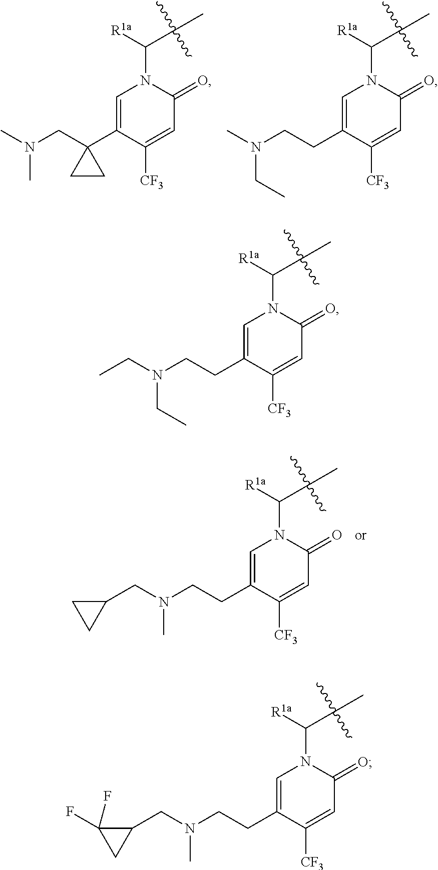

or R¹ is selected from

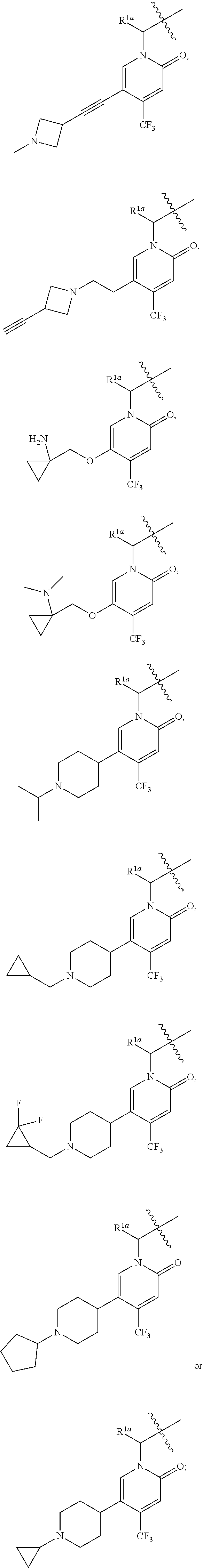

or R¹ is selected from

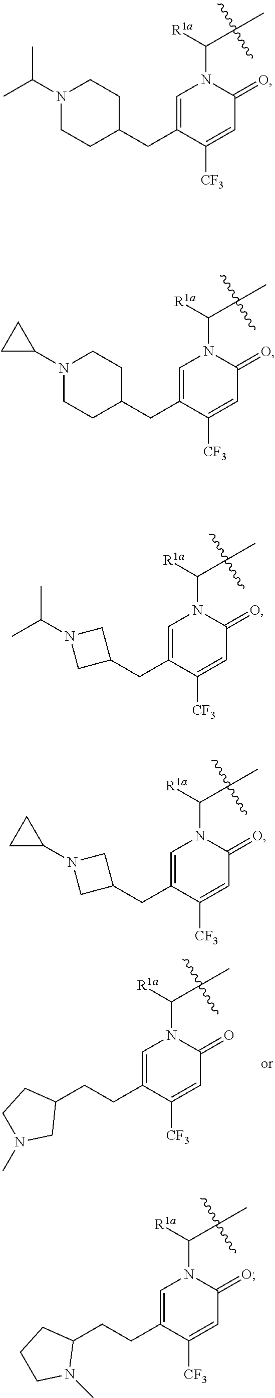

or R¹ is selected from

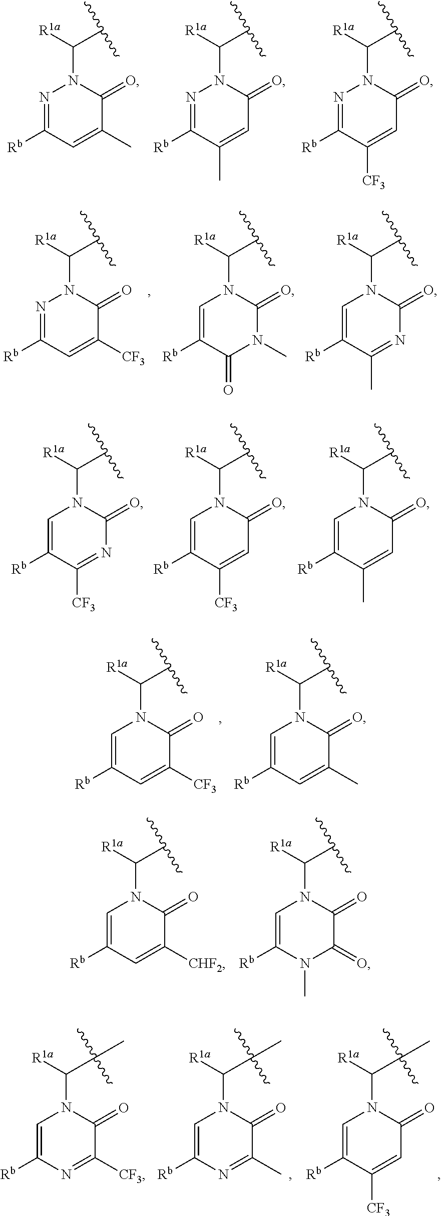

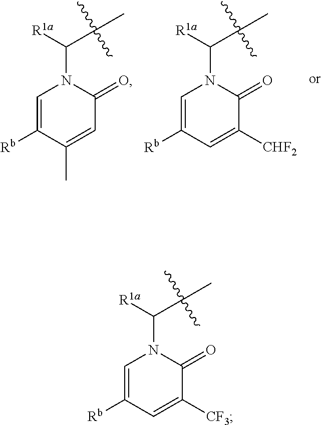

R² is selected from

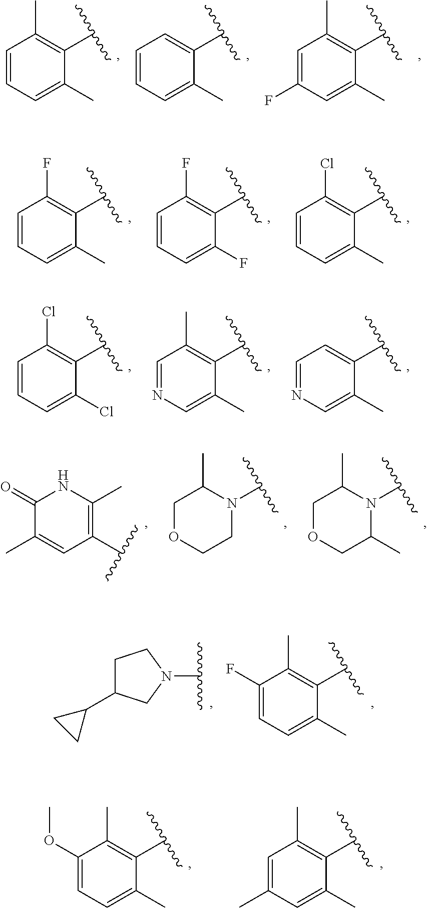

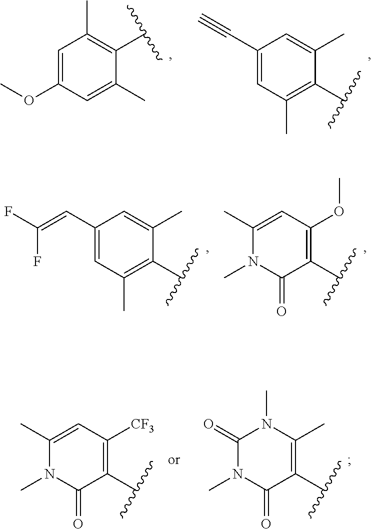

or R² is selected from

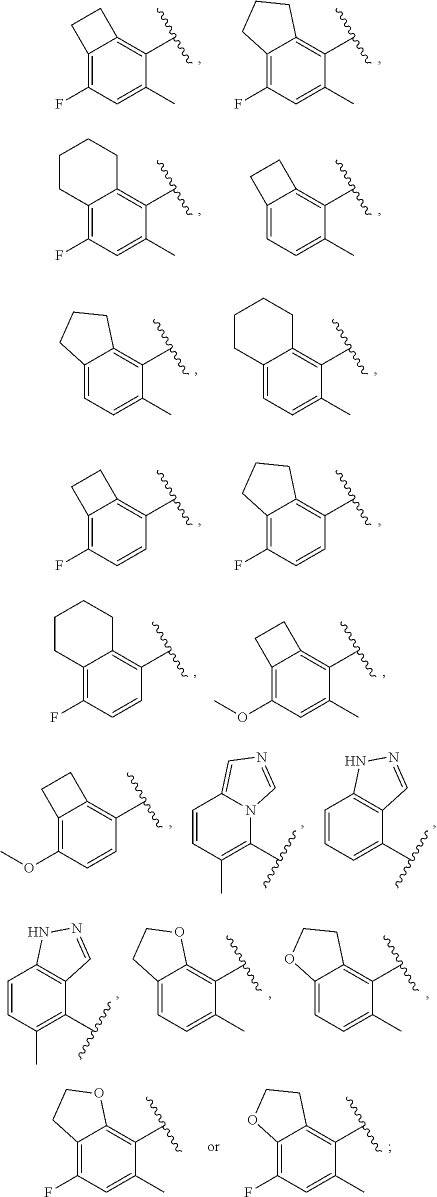

or R² is selected from

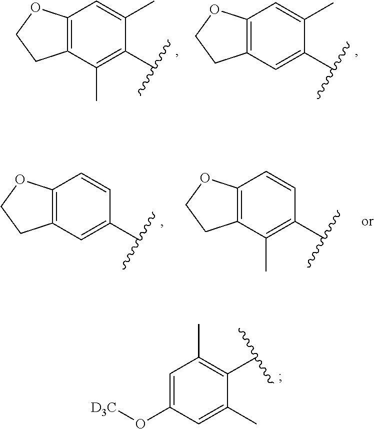 [SMILES: *c1c(C)cc2c(c1C)CCO2 ; *c1cc2c(cc1C)OCC2 ; *c1ccc2c(c1)CCO2 ; *c1ccc2c(c1C)CCO2 ; *c1c(C)cc(OC([2H])([2H])[2H])cc1C]

R^{1a} is selected from

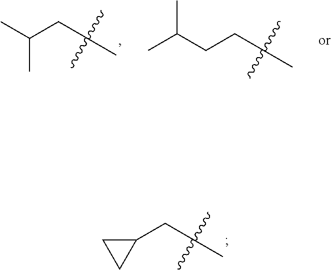

the definitions of the remaining substituents are the same as those in any one of the first, second, third, fourth or fifth embodiment of the present disclosure.

As a seventh embodiment of the present disclosure, provided is the compound of above general formula (I) or a stereoisomer, mesomer/racemate, deuterated product, solvate, prodrug, metabolite, pharmaceutically acceptable salt, or co-crystal thereof, wherein the compound is selected from a compound of general formula (I-a), (I-b), (I-a-1) or (I-b-1),

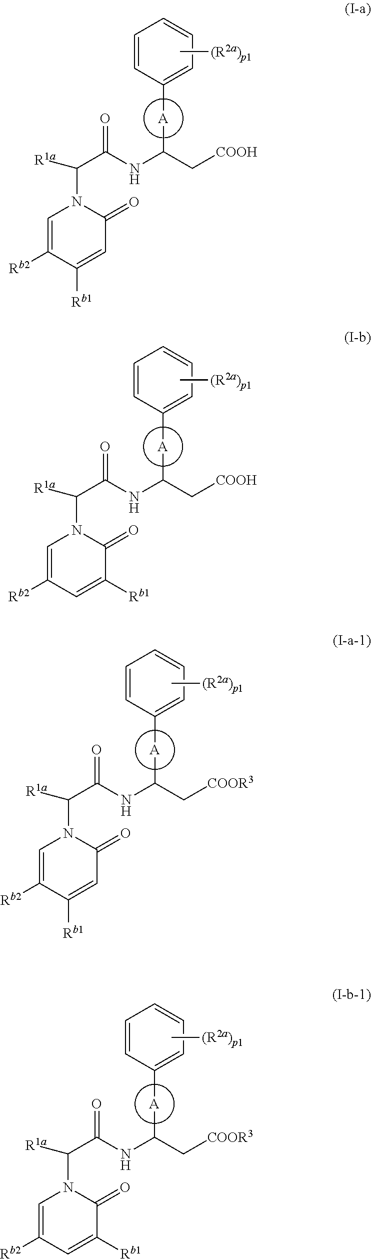

the definitions of ring A and R³ are the same as those in any one of the second, third, fourth, fifth or sixth embodiment of the present disclosure;
p1 is selected from 0, 1, 2, 3 or 4;
R^{2a} is selected from deuterium, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from deuterium, halogen, OH, CN, C₁₋₄ alkyl or C₁₋₄ alkoxy, and the heterocycloalkyl contains 1 to 4 heteroatoms selected from O, S or N;
R^{1a} is selected from

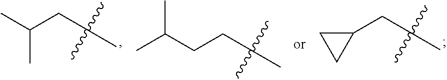

R^{b1} is selected from H, C₁₋₄ alkyl or C₃₋₆ cycloalkyl, wherein the alkyl or cycloalkyl is optionally substituted with 0 to 4 substituents selected from deuterium, halogen, OH, CN, NHC₁₋₄ alkyl, N(C₁₋₄ alkyl)₂, C₁₋₄ alkyl or C₁₋₄ alkoxy;
R^{b2} is selected from H, R^{ba}, or one of the following substituted or unsubstituted groups: C₁₋₄ alkyl, C₂₋₄ alkynyl, C₁₋₄ alkoxy, —CH₂CH₂—NHC₁₋₄ alkyl, —CH₂CH₂—N(C₁₋₄ alkyl)₂, —CH₂CH₂—C₃₋₆ cycloalkyl, —CH₂CH₂-3- to 7-membered heterocycloalkyl, wherein the CH₂, alkyl, cycloalkyl or heterocycloalkyl is optionally substituted with 0 to 4 substituents selected from deuterium, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from 0, S or N;
n1 is selected from 1, 2 or 3;
the definitions of R^{ba} and R^{k} are the same as those in any one of the second, third, fourth, fifth or sixth embodiment of the present disclosure.

As an eighth embodiment of the present disclosure, provided is the compound of above general formula (I) or a stereoisomer, mesomer/racemate, deuterated product, solvate, prodrug, metabolite, pharmaceutically acceptable salt, or co-crystal thereof, wherein the compound is selected from a compound of general formula (I-a), (I-b), (I-a-1) or (I-b-1),
R^{2a} is selected from deuterium, CD₃, —OCD₃, F, Cl, Br, I, OH, cyano, methyl, ethyl, methoxy or ethoxy;
R^{b1} is selected from H, F, CH₂F, CHF₂, CF₃, methyl or —CH₂CH₂N(CH₃)₂;
R^{b2} is selected from H, R^{ba}, or one of the following substituted or unsubstituted groups: methyl, ethyl, ethynyl, methoxy, ethoxy, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl or —CH₂CH₂-morpholine, which, when substituted, is substituted with 1, 2 or 3 substituents selected from deuterium, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
the definitions of the remaining groups are the same as those in the seventh embodiment of the present disclosure.

As a ninth embodiment of the present disclosure, provided is the compound of above general formula (I) or a stereoisomer, mesomer/racemate, deuterated product, solvate, prodrug, metabolite, pharmaceutically acceptable salt, or co-crystal thereof, wherein the compound is selected from a compound of general formula (I-f), (I-g), (I-f-1), (I-g-1), (I-h), (I-i), (I-h-1) or (I-i-1):

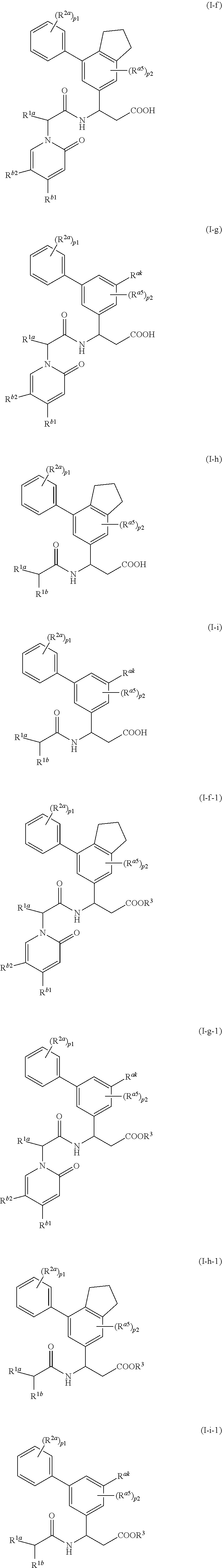

R^{1b} is selected from

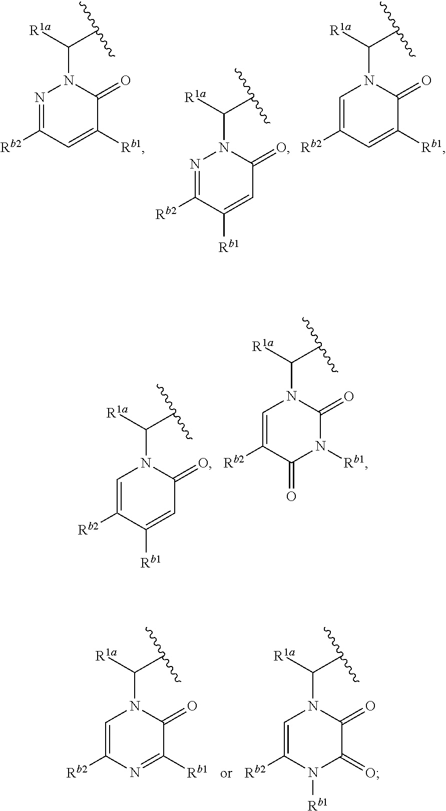

wherein p2 is selected from 0, 1 or 2;
R^{ak} is selected from C₂₋₄ alkynyl;
the definition of R^{a5} is the same as that in any one of the first, second, third or fourth embodiment of the present disclosure;
the definitions of the remaining groups are the same as those in the seventh or eighth embodiment of the present disclosure.

As a tenth embodiment of the present disclosure, provided is the compound of above general formula (I) or a stereoisomer, mesomer/racemate, deuterated product, solvate, prodrug, metabolite, pharmaceutically acceptable salt, or co-crystal thereof, wherein the compound is selected from a compound of general formula (I-f), (I-g), (I-f-1), (I-g-1), (I-h), (I-i), (I-h-1) or (I-i-1);
R^{k} is selected from ethynyl, propynyl or propargyl;
R^{a5} is selected from F, Cl, Br, I, OH, cyano, methyl, ethyl, methoxy, ethoxy or ethynyl;
the definitions of the remaining groups are the same as those in the ninth embodiment of the present disclosure.

As a tenth embodiment of the present disclosure, provided is the compound of above general formula (I) or a stereoisomer, mesomer/racemate, deuterated product, solvate, prodrug, metabolite, pharmaceutically acceptable salt, or co-crystal thereof, wherein the compound is selected from a compound of general formula (I-j) or (I-k);

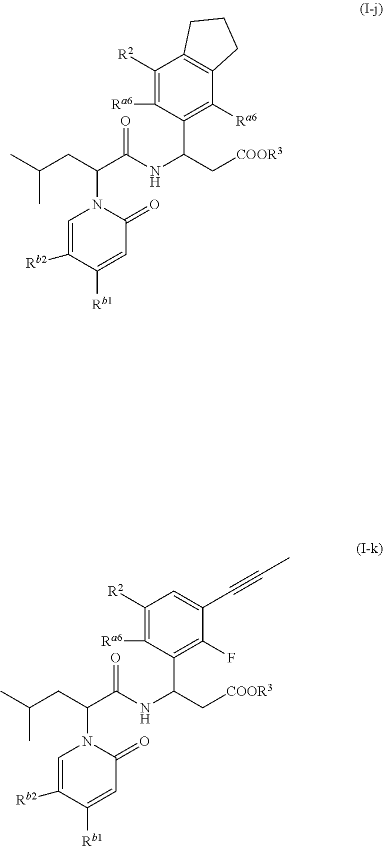

R^{a6} is selected from H or F;
R² is selected from phenyl or

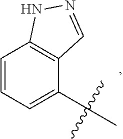 [SMILES: *c1cccc2[nH]ncc12]

and the R² is optionally substituted with 1 to 4 substituents selected from halogen, C₁₋₄ alkyl, C₁₋₄ alkoxy, 1 to 4 halogen(s)-substituted C₁₋₄ alkyl, 1 to 4 halogen(s)-substituted C₁₋₄ alkoxy, 1 to 4 deuterium(s)-substituted C₁₋₄ alkyl or 1 to 4 deuterium(s)-substituted C₁₋₄ alkoxy, preferably,

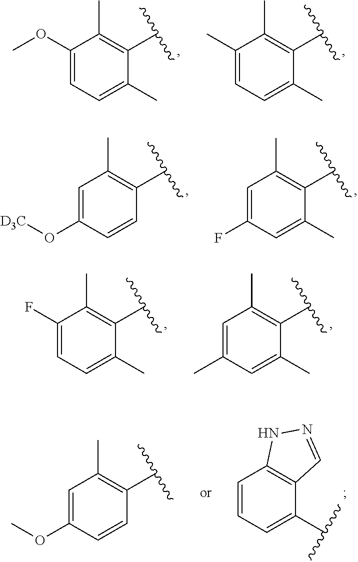

R^{b1} is selected from CH₂F, CHF₂, CF₃ or methyl, preferably CF₃;
R^{b2} is selected from —CH₂CH₂N(CH₃)₂, —CH₂CH₂NH(CH₂CH₃), —CH₂-azetidinyl, —CH₂-azolidinyl, —CH₂-azinanyl, —CH₂-morpholinyl, —CH₂CH₂-azetidinyl, —CH₂CH₂-azolidinyl, —CH₂CH₂-azinanyl or

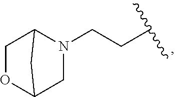 [SMILES: *CCN1CC2CC1CO2]

and the R^{b2} is optionally substituted with 1 to 4 substituents selected from deuterium, F, Cl, Br, NHCH₂CH₃, N(CH₂CH₃)₂, CH₂F, CHF₂, CF₃, methyl, ethyl, isopropyl, methoxy, ethoxy, —CH₂-cyclopropyl or

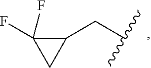 [SMILES: *CC1CC1(F)F]

preferably

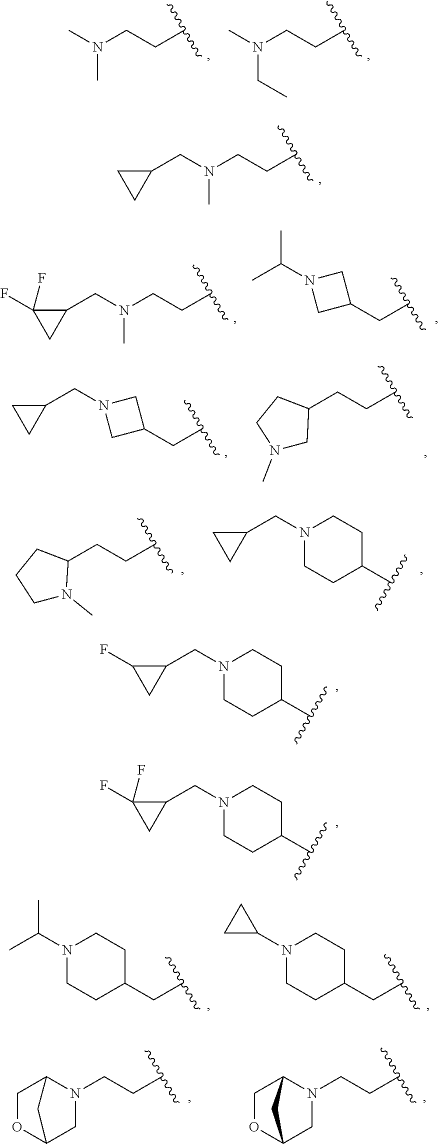

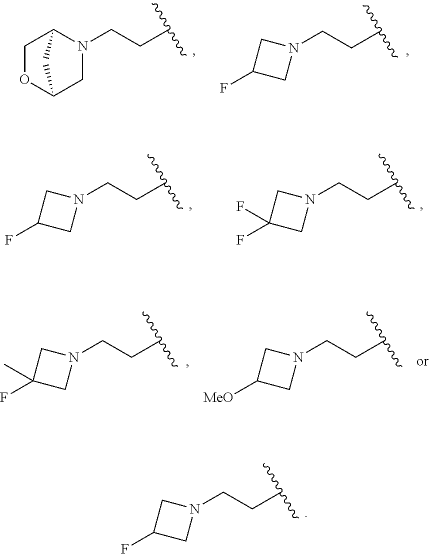

The present disclosure relates to some specific compounds of general formula (I), wherein the compounds are selected from Table E-1.

The present disclosure relates to some specific compounds of general formula (I), wherein the compounds are selected from Table E-3, Table E-4 or table E-5.

TABLE E-1

| | |
|---|---|
| 1 | 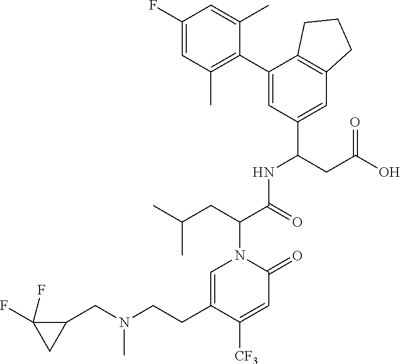 [SMILES: Cc1cc(F)cc(C)c1-c1cc(C(CC(=O)O)NC(=O)C(CC(C)C)n2cc(CCN(C)CC3CC3(F)F)c(C(F)(F)F)cc2=O)cc2c1CCC2] |
| | 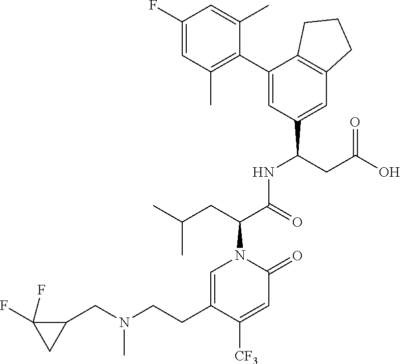 [SMILES: Cc1cc(F)cc(C)c1-c1cc([C@@H](CC(=O)O)NC(=O)[C@H](CC(C)C)n2cc(CCN(C)CC3CC3(F)F)c(C(F)(F)F)cc2=O)cc2c1CCC2] |
| | 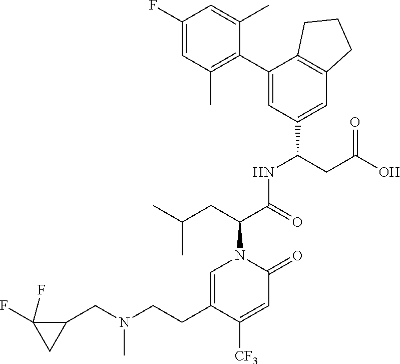 [SMILES: Cc1cc(F)cc(C)c1-c1cc([C@H](CC(=O)O)NC(=O)[C@H](CC(C)C)n2cc(CCN(C)CC3CC3(F)F)c(C(F)(F)F)cc2=O)cc2c1CCC2] |
| | 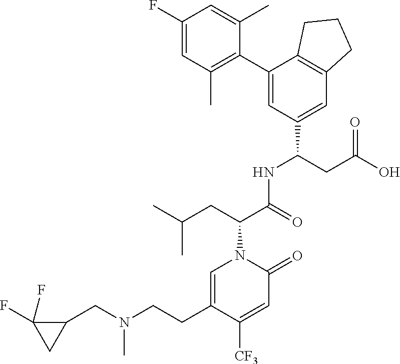 [SMILES: Cc1cc(F)cc(C)c1-c1cc([C@H](CC(=O)O)NC(=O)[C@@H](CC(C)C)n2cc(CCN(C)CC3CC3(F)F)c(C(F)(F)F)cc2=O)cc2c1CCC2] |
| | 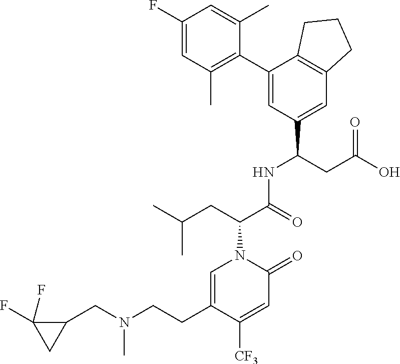 [SMILES: Cc1cc(F)cc(C)c1-c1cc([C@@H](CC(=O)O)NC(=O)[C@@H](CC(C)C)n2cc(CCN(C)CC3CC3(F)F)c(C(F)(F)F)cc2=O)cc2c1CCC2] |
| 2 | 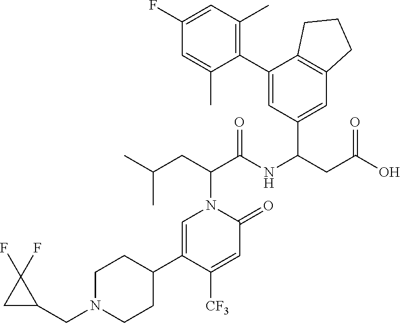 [SMILES: Cc1cc(F)cc(C)c1-c1cc(C(CC(=O)O)NC(=O)C(CC(C)C)n2cc(C3CCN(CC4CC4(F)F)CC3)c(C(F)(F)F)cc2=O)cc2c1CCC2] |
| | 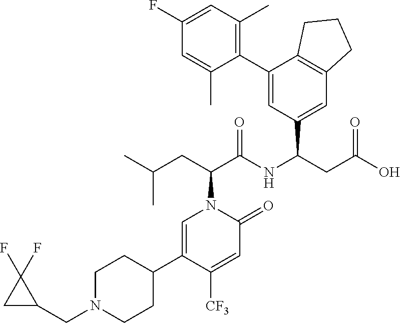 [SMILES: Cc1cc(F)cc(C)c1-c1cc([C@@H](CC(=O)O)NC(=O)[C@H](CC(C)C)n2cc(C3CCN(CC4CC4(F)F)CC3)c(C(F)(F)F)cc2=O)cc2c1CCC2] |
| | 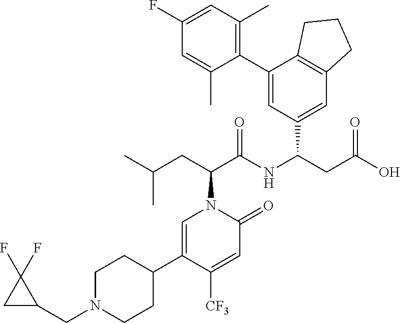 [SMILES: Cc1cc(F)cc(C)c1-c1cc([C@H](CC(=O)O)NC(=O)[C@H](CC(C)C)n2cc(C3CCN(CC4CC4(F)F)CC3)c(C(F)(F)F)cc2=O)cc2c1CCC2] |
| | 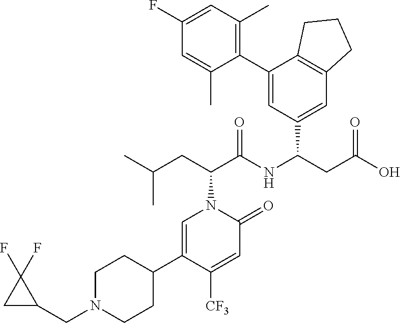 [SMILES: Cc1cc(F)cc(C)c1-c1cc([C@H](CC(=O)O)NC(=O)[C@@H](CC(C)C)n2cc(C3CCN(CC4CC4(F)F)CC3)c(C(F)(F)F)cc2=O)cc2c1CCC2] |
| | 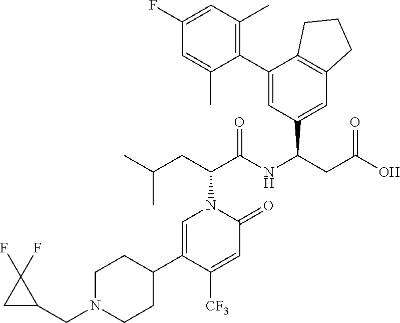 [SMILES: Cc1cc(F)cc(C)c1-c1cc([C@@H](CC(=O)O)NC(=O)[C@@H](CC(C)C)n2cc(C3CCN(CC4CC4(F)F)CC3)c(C(F)(F)F)cc2=O)cc2c1CCC2] |
| 3 | 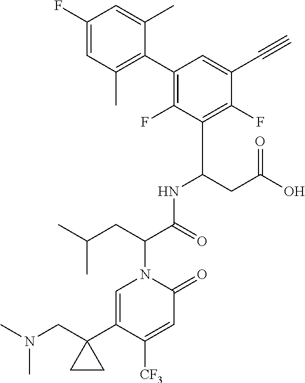 [SMILES: C#Cc1cc(-c2c(C)cc(F)cc2C)c(F)c(C(CC(=O)O)NC(=O)C(CC(C)C)n2cc(C3(CN(C)C)CC3)c(C(F)(F)F)cc2=O)c1F] |
| | 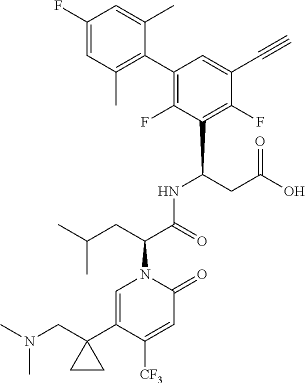 [SMILES: C#Cc1cc(-c2c(C)cc(F)cc2C)c(F)c([C@@H](CC(=O)O)NC(=O)[C@H](CC(C)C)n2cc(C3(CN(C)C)CC3)c(C(F)(F)F)cc2=O)c1F] |
| | 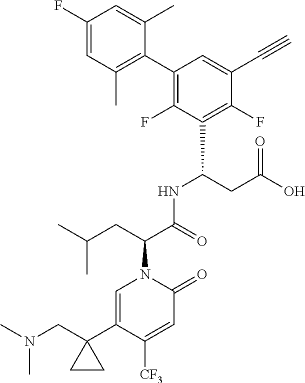 [SMILES: C#Cc1cc(-c2c(C)cc(F)cc2C)c(F)c([C@H](CC(=O)O)NC(=O)[C@H](CC(C)C)n2cc(C3(CN(C)C)CC3)c(C(F)(F)F)cc2=O)c1F] |
| | 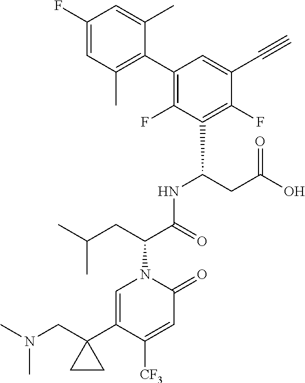 [SMILES: C#Cc1cc(-c2c(C)cc(F)cc2C)c(F)c([C@H](CC(=O)O)NC(=O)[C@@H](CC(C)C)n2cc(C3(CN(C)C)CC3)c(C(F)(F)F)cc2=O)c1F] |
| | 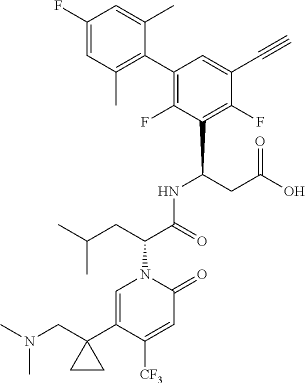 [SMILES: C#Cc1cc(-c2c(C)cc(F)cc2C)c(F)c([C@@H](CC(=O)O)NC(=O)[C@@H](CC(C)C)n2cc(C3(CN(C)C)CC3)c(C(F)(F)F)cc2=O)c1F] |
| 4 | 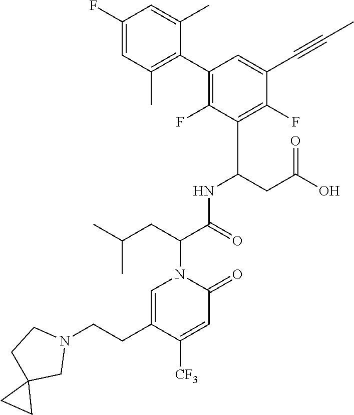 [SMILES: CC#Cc1cc(-c2c(C)cc(F)cc2C)c(F)c(C(CC(=O)O)NC(=O)C(CC(C)C)n2cc(CCN3CCC4(CC4)C3)c(C(F)(F)F)cc2=O)c1F] |
| | 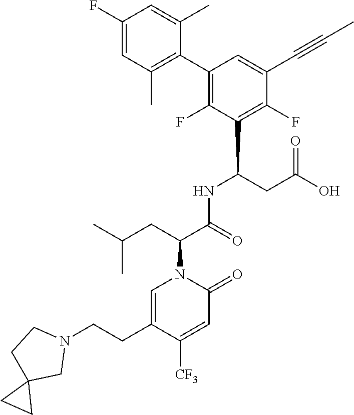 [SMILES: CC#Cc1cc(-c2c(C)cc(F)cc2C)c(F)c([C@@H](CC(=O)O)NC(=O)[C@H](CC(C)C)n2cc(CCN3CCC4(CC4)C3)c(C(F)(F)F)cc2=O)c1F] |
| | 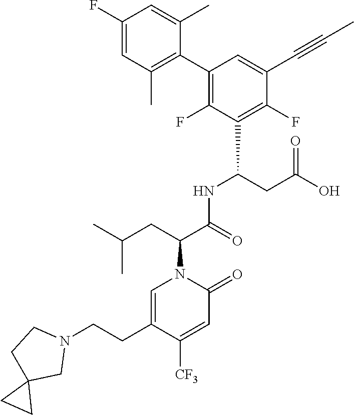 [SMILES: CC#Cc1cc(-c2c(C)cc(F)cc2C)c(F)c([C@H](CC(=O)O)NC(=O)[C@H](CC(C)C)n2cc(CCN3CCC4(CC4)C3)c(C(F)(F)F)cc2=O)c1F] |
| | 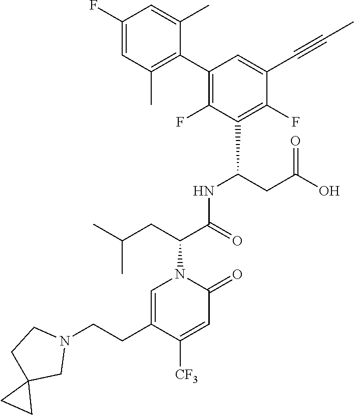 [SMILES: CC#Cc1cc(-c2c(C)cc(F)cc2C)c(F)c([C@H](CC(=O)O)NC(=O)[C@@H](CC(C)C)n2cc(CCN3CCC4(CC4)C3)c(C(F)(F)F)cc2=O)c1F] |
| | 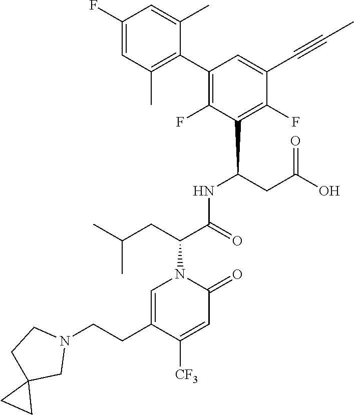 [SMILES: CC#Cc1cc(-c2c(C)cc(F)cc2C)c(F)c([C@@H](CC(=O)O)NC(=O)[C@@H](CC(C)C)n2cc(CCN3CCC4(CC4)C3)c(C(F)(F)F)cc2=O)c1F] |
| 5 | 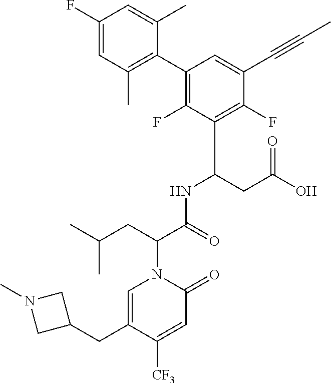 [SMILES: CC#Cc1cc(-c2c(C)cc(F)cc2C)c(F)c(C(CC(=O)O)NC(=O)C(CC(C)C)n2cc(CC3CN(C)C3)c(C(F)(F)F)cc2=O)c1F] |
| | 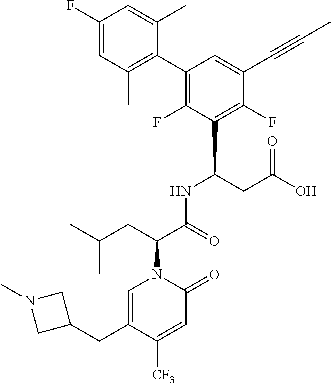 [SMILES: CC#Cc1cc(-c2c(C)cc(F)cc2C)c(F)c([C@@H](CC(=O)O)NC(=O)[C@H](CC(C)C)n2cc(CC3CN(C)C3)c(C(F)(F)F)cc2=O)c1F] |
| | 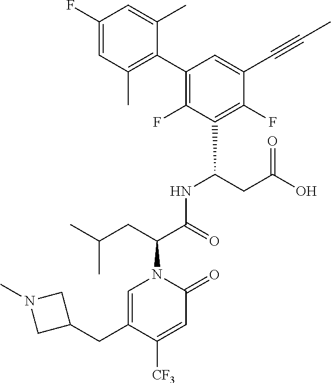 [SMILES: CC#Cc1cc(-c2c(C)cc(F)cc2C)c(F)c([C@H](CC(=O)O)NC(=O)[C@H](CC(C)C)n2cc(CC3CN(C)C3)c(C(F)(F)F)cc2=O)c1F] |
| | 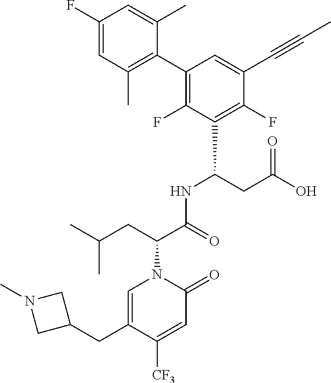 [SMILES: CC#Cc1cc(-c2c(C)cc(F)cc2C)c(F)c([C@H](CC(=O)O)NC(=O)[C@@H](CC(C)C)n2cc(CC3CN(C)C3)c(C(F)(F)F)cc2=O)c1F] |
| | 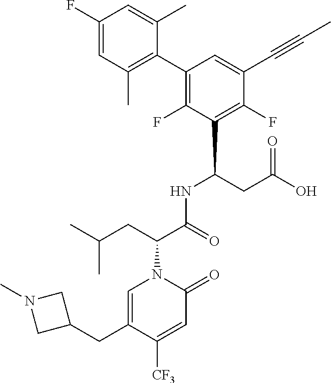 [SMILES: CC#Cc1cc(-c2c(C)cc(F)cc2C)c(F)c([C@@H](CC(=O)O)NC(=O)[C@@H](CC(C)C)n2cc(CC3CN(C)C3)c(C(F)(F)F)cc2=O)c1F] |
| 6 | 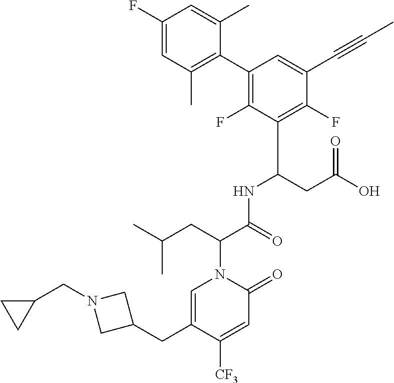 [SMILES: CC#Cc1cc(-c2c(C)cc(F)cc2C)c(F)c(C(CC(=O)O)NC(=O)C(CC(C)C)n2cc(CC3CN(CC4CC4)C3)c(C(F)(F)F)cc2=O)c1F] |
| | 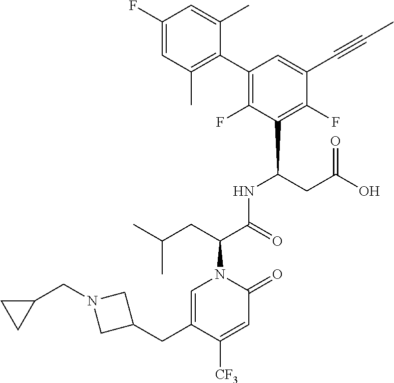 [SMILES: CC#Cc1cc(-c2c(C)cc(F)cc2C)c(F)c([C@@H](CC(=O)O)NC(=O)[C@H](CC(C)C)n2cc(CC3CN(CC4CC4)C3)c(C(F)(F)F)cc2=O)c1F] |
| | 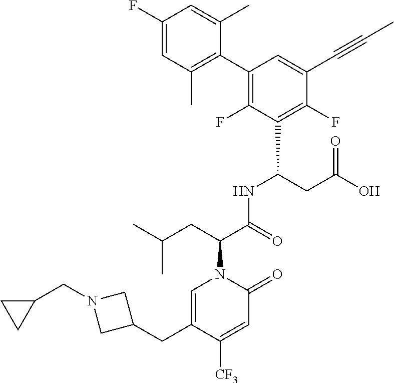 [SMILES: CC#Cc1cc(-c2c(C)cc(F)cc2C)c(F)c([C@H](CC(=O)O)NC(=O)[C@H](CC(C)C)n2cc(CC3CN(CC4CC4)C3)c(C(F)(F)F)cc2=O)c1F] |
| | 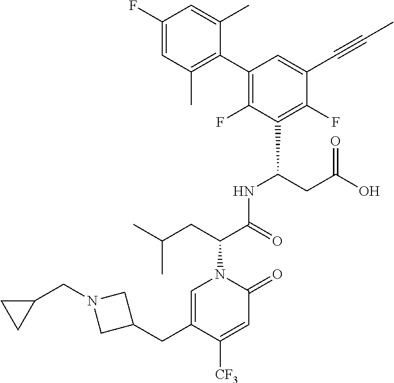 [SMILES: CC#Cc1cc(-c2c(C)cc(F)cc2C)c(F)c([C@H](CC(=O)O)NC(=O)[C@@H](CC(C)C)n2cc(CC3CN(CC4CC4)C3)c(C(F)(F)F)cc2=O)c1F] |
| | 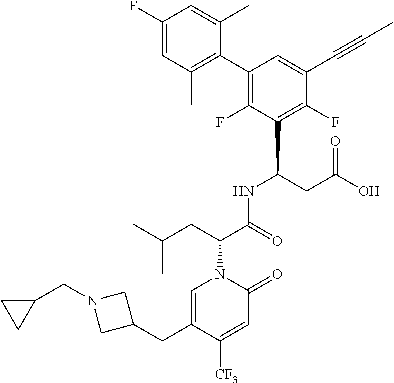 [SMILES: CC#Cc1cc(-c2c(C)cc(F)cc2C)c(F)c([C@@H](CC(=O)O)NC(=O)[C@@H](CC(C)C)n2cc(CC3CN(CC4CC4)C3)c(C(F)(F)F)cc2=O)c1F] |
| 7 | 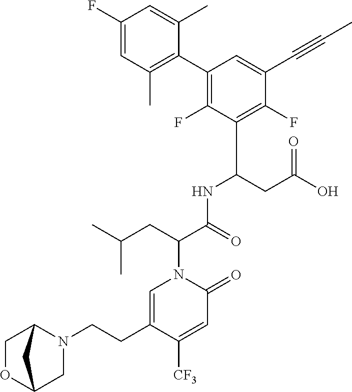 [SMILES: CC#Cc1cc(-c2c(C)cc(F)cc2C)c(F)c(C(CC(=O)O)NC(=O)C(CC(C)C)n2cc(CCN3C[C@H]4C[C@@H]3CO4)c(C(F)(F)F)cc2=O)c1F] |
| | 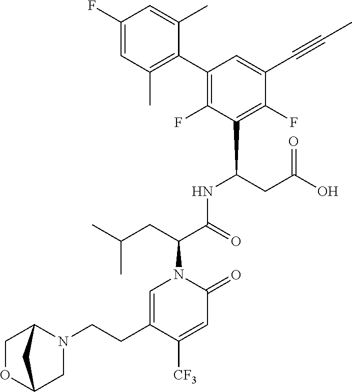 [SMILES: CC#Cc1cc(-c2c(C)cc(F)cc2C)c(F)c([C@@H](CC(=O)O)NC(=O)[C@H](CC(C)C)n2cc(CCN3C[C@H]4C[C@@H]3CO4)c(C(F)(F)F)cc2=O)c1F] |
| | 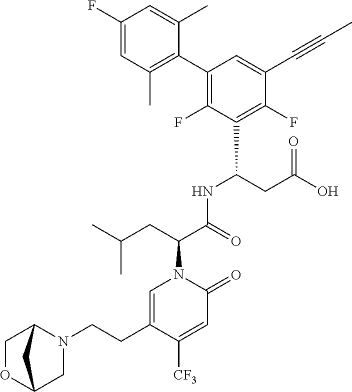 [SMILES: CC#Cc1cc(-c2c(C)cc(F)cc2C)c(F)c([C@H](CC(=O)O)NC(=O)[C@H](CC(C)C)n2cc(CCN3C[C@H]4C[C@@H]3CO4)c(C(F)(F)F)cc2=O)c1F] |
| | 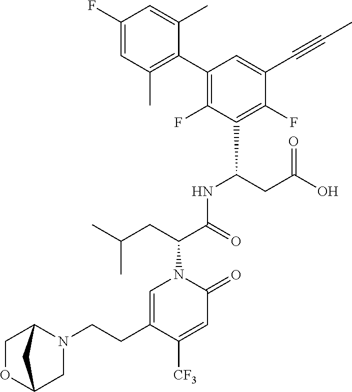 [SMILES: CC#Cc1cc(-c2c(C)cc(F)cc2C)c(F)c([C@H](CC(=O)O)NC(=O)[C@@H](CC(C)C)n2cc(CCN3C[C@H]4C[C@@H]3CO4)c(C(F)(F)F)cc2=O)c1F] |
| | 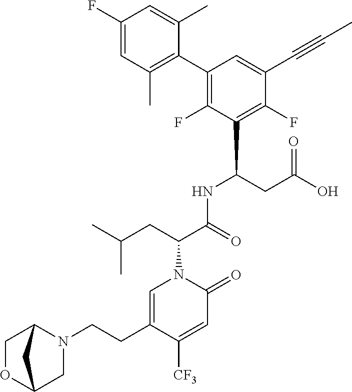 [SMILES: CC#Cc1cc(-c2c(C)cc(F)cc2C)c(F)c([C@@H](CC(=O)O)NC(=O)[C@@H](CC(C)C)n2cc(CCN3C[C@H]4C[C@@H]3CO4)c(C(F)(F)F)cc2=O)c1F] |
| 8 | 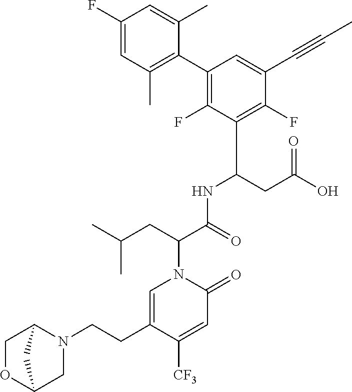 [SMILES: CC#Cc1cc(-c2c(C)cc(F)cc2C)c(F)c(C(CC(=O)O)NC(=O)C(CC(C)C)n2cc(CCN3C[C@@H]4C[C@H]3CO4)c(C(F)(F)F)cc2=O)c1F] |
| | 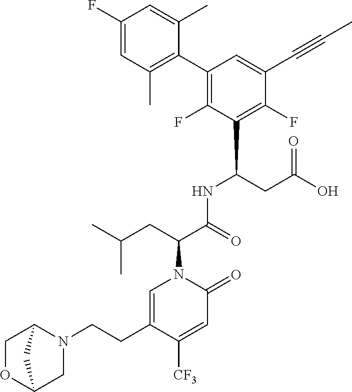 [SMILES: CC#Cc1cc(-c2c(C)cc(F)cc2C)c(F)c([C@@H](CC(=O)O)NC(=O)[C@H](CC(C)C)n2cc(CCN3C[C@@H]4C[C@H]3CO4)c(C(F)(F)F)cc2=O)c1F] |
| | 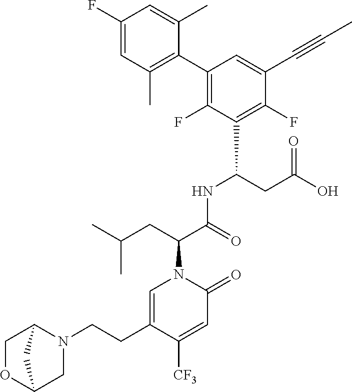 [SMILES: CC#Cc1cc(-c2c(C)cc(F)cc2C)c(F)c([C@H](CC(=O)O)NC(=O)[C@H](CC(C)C)n2cc(CCN3C[C@@H]4C[C@H]3CO4)c(C(F)(F)F)cc2=O)c1F] |
| | 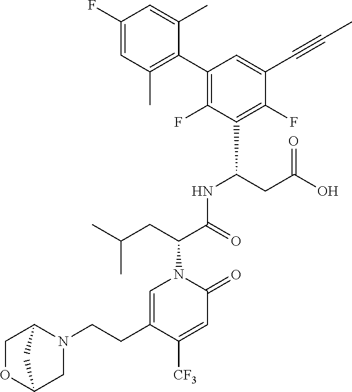 [SMILES: CC#Cc1cc(-c2c(C)cc(F)cc2C)c(F)c([C@H](CC(=O)O)NC(=O)[C@@H](CC(C)C)n2cc(CCN3C[C@@H]4C[C@H]3CO4)c(C(F)(F)F)cc2=O)c1F] |
| | 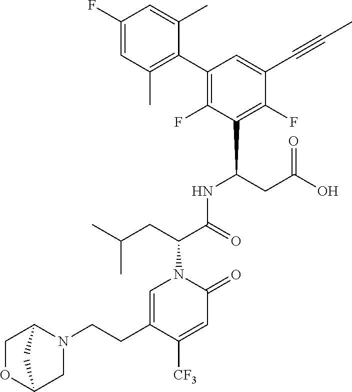 [SMILES: CC#Cc1cc(-c2c(C)cc(F)cc2C)c(F)c([C@@H](CC(=O)O)NC(=O)[C@@H](CC(C)C)n2cc(CCN3C[C@@H]4C[C@H]3CO4)c(C(F)(F)F)cc2=O)c1F] |
| 9 | 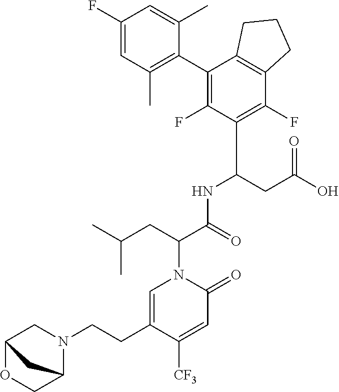 [SMILES: Cc1cc(F)cc(C)c1-c1c(F)c(C(CC(=O)O)NC(=O)C(CC(C)C)n2cc(CCN3C[C@@H]4C[C@H]3CO4)c(C(F)(F)F)cc2=O)c(F)c2c1CCC2] |
| | 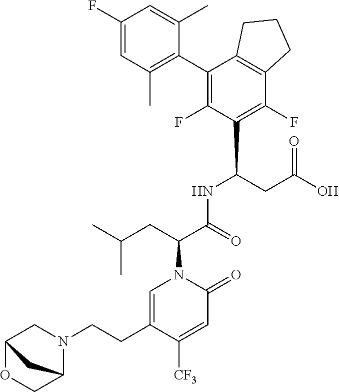 [SMILES: Cc1cc(F)cc(C)c1-c1c(F)c([C@@H](CC(=O)O)NC(=O)[C@H](CC(C)C)n2cc(CCN3C[C@@H]4C[C@H]3CO4)c(C(F)(F)F)cc2=O)c(F)c2c1CCC2] |
| | 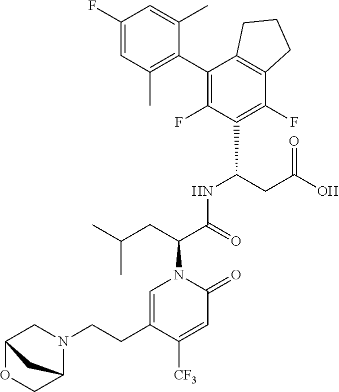 [SMILES: Cc1cc(F)cc(C)c1-c1c(F)c([C@H](CC(=O)O)NC(=O)[C@H](CC(C)C)n2cc(CCN3C[C@@H]4C[C@H]3CO4)c(C(F)(F)F)cc2=O)c(F)c2c1CCC2] |
| | 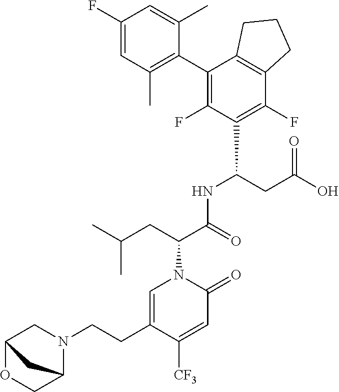 [SMILES: Cc1cc(F)cc(C)c1-c1c(F)c([C@H](CC(=O)O)NC(=O)[C@@H](CC(C)C)n2cc(CCN3C[C@@H]4C[C@H]3CO4)c(C(F)(F)F)cc2=O)c(F)c2c1CCC2] |
| | 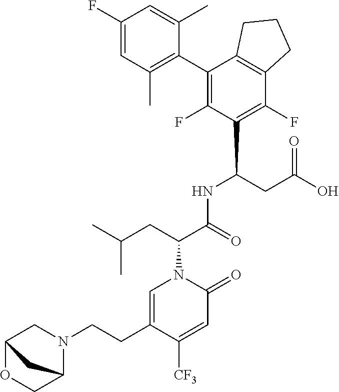 [SMILES: Cc1cc(F)cc(C)c1-c1c(F)c([C@@H](CC(=O)O)NC(=O)[C@@H](CC(C)C)n2cc(CCN3C[C@@H]4C[C@H]3CO4)c(C(F)(F)F)cc2=O)c(F)c2c1CCC2] |
| 10 | 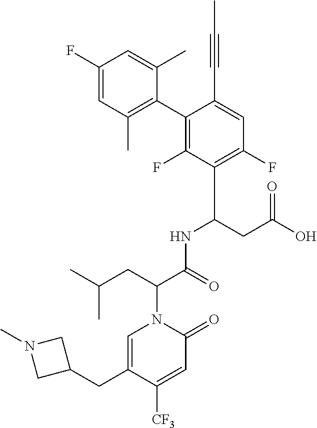 [SMILES: CC#Cc1cc(F)c(C(CC(=O)O)NC(=O)C(CC(C)C)n2cc(CC3CN(C)C3)c(C(F)(F)F)cc2=O)c(F)c1-c1c(C)cc(F)cc1C] |
| | 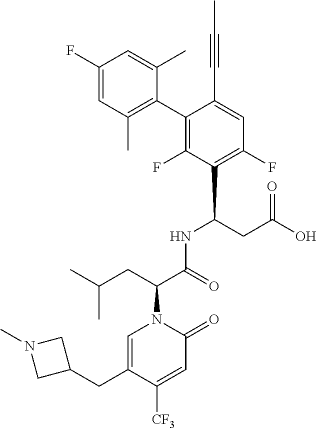 [SMILES: CC#Cc1cc(F)c([C@@H](CC(=O)O)NC(=O)[C@H](CC(C)C)n2cc(CC3CN(C)C3)c(C(F)(F)F)cc2=O)c(F)c1-c1c(C)cc(F)cc1C] |
| | 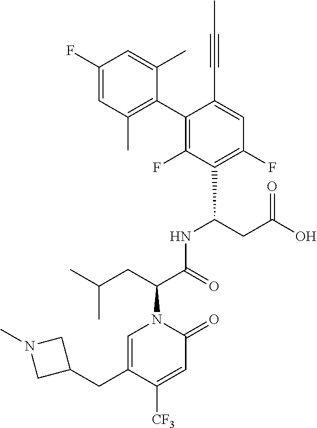 [SMILES: CC#Cc1cc(F)c([C@H](CC(=O)O)NC(=O)[C@H](CC(C)C)n2cc(CC3CN(C)C3)c(C(F)(F)F)cc2=O)c(F)c1-c1c(C)cc(F)cc1C] |
| | 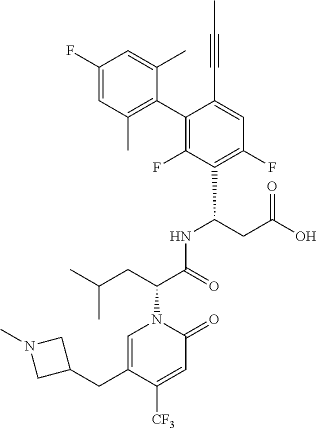 [SMILES: CC#Cc1cc(F)c([C@H](CC(=O)O)NC(=O)[C@@H](CC(C)C)n2cc(CC3CN(C)C3)c(C(F)(F)F)cc2=O)c(F)c1-c1c(C)cc(F)cc1C] |
| | 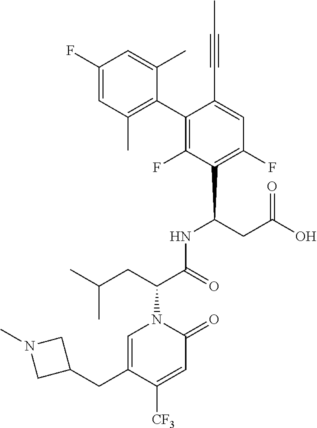 [SMILES: CC#Cc1cc(F)c([C@@H](CC(=O)O)NC(=O)[C@@H](CC(C)C)n2cc(CC3CN(C)C3)c(C(F)(F)F)cc2=O)c(F)c1-c1c(C)cc(F)cc1C] |
| 11 | 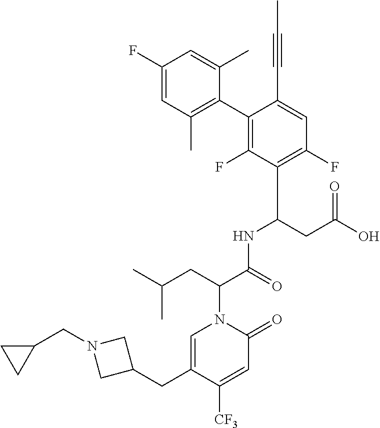 [SMILES: CC#Cc1cc(F)c(C(CC(=O)O)NC(=O)C(CC(C)C)n2cc(CC3CN(CC4CC4)C3)c(C(F)(F)F)cc2=O)c(F)c1-c1c(C)cc(F)cc1C] |
| | 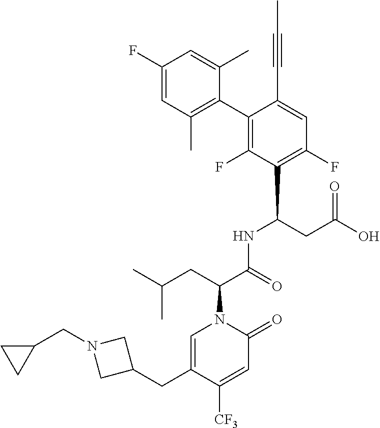 [SMILES: CC#Cc1cc(F)c([C@@H](CC(=O)O)NC(=O)[C@H](CC(C)C)n2cc(CC3CN(CC4CC4)C3)c(C(F)(F)F)cc2=O)c(F)c1-c1c(C)cc(F)cc1C] |
| | 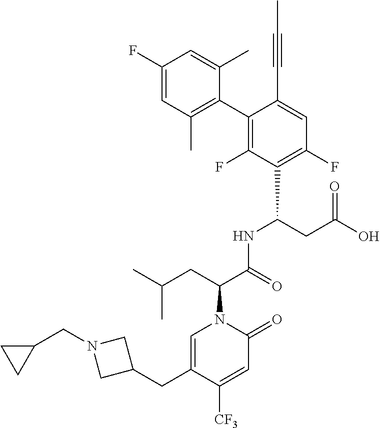 [SMILES: CC#Cc1cc(F)c([C@H](CC(=O)O)NC(=O)[C@H](CC(C)C)n2cc(CC3CN(CC4CC4)C3)c(C(F)(F)F)cc2=O)c(F)c1-c1c(C)cc(F)cc1C] |
| | 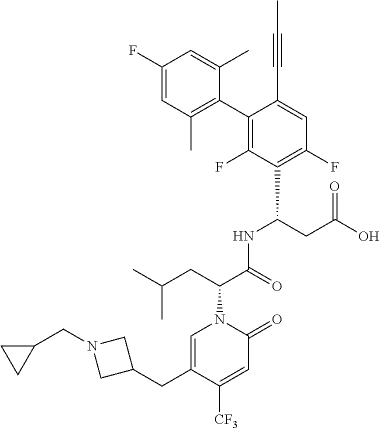 [SMILES: CC#Cc1cc(F)c([C@H](CC(=O)O)NC(=O)[C@@H](CC(C)C)n2cc(CC3CN(CC4CC4)C3)c(C(F)(F)F)cc2=O)c(F)c1-c1c(C)cc(F)cc1C] |
| | 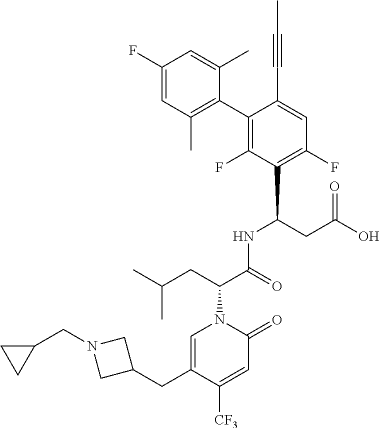 [SMILES: CC#Cc1cc(F)c([C@@H](CC(=O)O)NC(=O)[C@@H](CC(C)C)n2cc(CC3CN(CC4CC4)C3)c(C(F)(F)F)cc2=O)c(F)c1-c1c(C)cc(F)cc1C] |
| 12 | 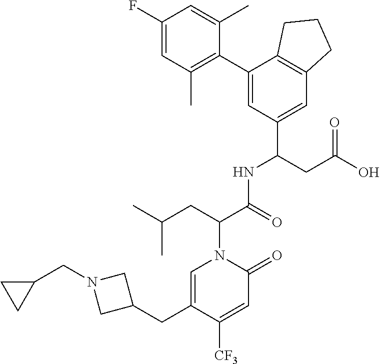 [SMILES: Cc1cc(F)cc(C)c1-c1cc(C(CC(=O)O)NC(=O)C(CC(C)C)n2cc(CC3CN(CC4CC4)C3)c(C(F)(F)F)cc2=O)cc2c1CCC2] |
| | 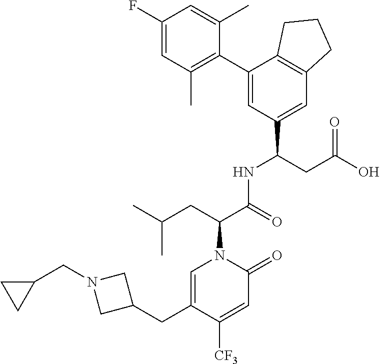 [SMILES: Cc1cc(F)cc(C)c1-c1cc([C@@H](CC(=O)O)NC(=O)[C@H](CC(C)C)n2cc(CC3CN(CC4CC4)C3)c(C(F)(F)F)cc2=O)cc2c1CCC2] |
| | 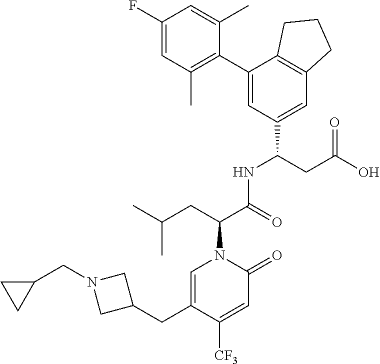 [SMILES: Cc1cc(F)cc(C)c1-c1cc([C@H](CC(=O)O)NC(=O)[C@H](CC(C)C)n2cc(CC3CN(CC4CC4)C3)c(C(F)(F)F)cc2=O)cc2c1CCC2] |
| | 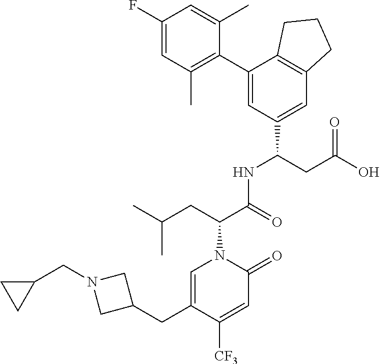 [SMILES: Cc1cc(F)cc(C)c1-c1cc([C@H](CC(=O)O)NC(=O)[C@@H](CC(C)C)n2cc(CC3CN(CC4CC4)C3)c(C(F)(F)F)cc2=O)cc2c1CCC2] |
| | 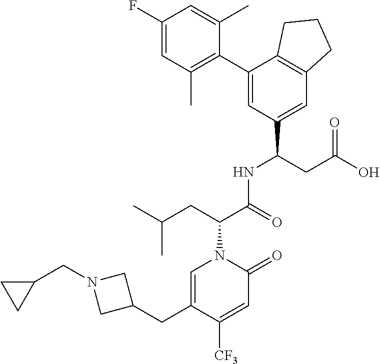 [SMILES: Cc1cc(F)cc(C)c1-c1cc([C@@H](CC(=O)O)NC(=O)[C@@H](CC(C)C)n2cc(CC3CN(CC4CC4)C3)c(C(F)(F)F)cc2=O)cc2c1CCC2] |
| 13 | 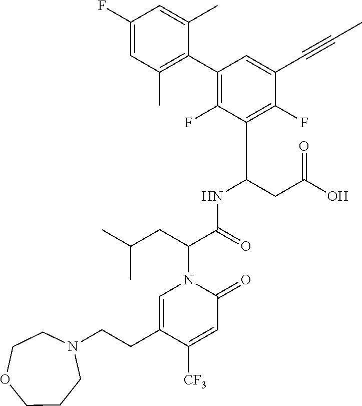 [SMILES: CC#Cc1cc(-c2c(C)cc(F)cc2C)c(F)c(C(CC(=O)O)NC(=O)C(CC(C)C)n2cc(CCN3CCCOCC3)c(C(F)(F)F)cc2=O)c1F] |
| | 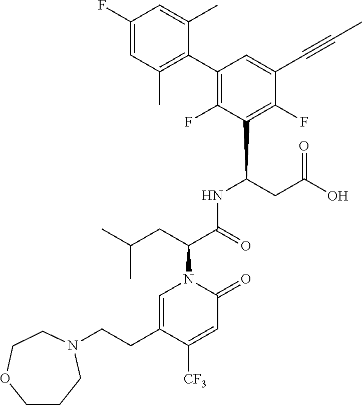 [SMILES: CC#Cc1cc(-c2c(C)cc(F)cc2C)c(F)c([C@@H](CC(=O)O)NC(=O)[C@H](CC(C)C)n2cc(CCN3CCCOCC3)c(C(F)(F)F)cc2=O)c1F] |
| | 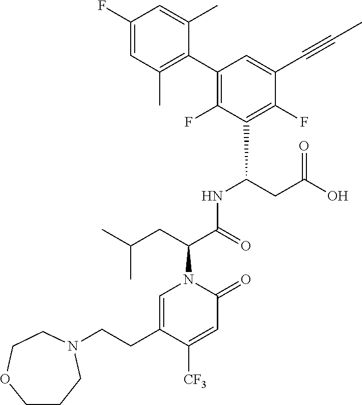 [SMILES: CC#Cc1cc(-c2c(C)cc(F)cc2C)c(F)c([C@H](CC(=O)O)NC(=O)[C@H](CC(C)C)n2cc(CCN3CCCOCC3)c(C(F)(F)F)cc2=O)c1F] |
| | 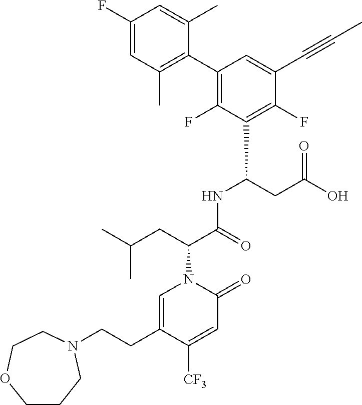 [SMILES: CC#Cc1cc(-c2c(C)cc(F)cc2C)c(F)c([C@H](CC(=O)O)NC(=O)[C@@H](CC(C)C)n2cc(CCN3CCCOCC3)c(C(F)(F)F)cc2=O)c1F] |
| | 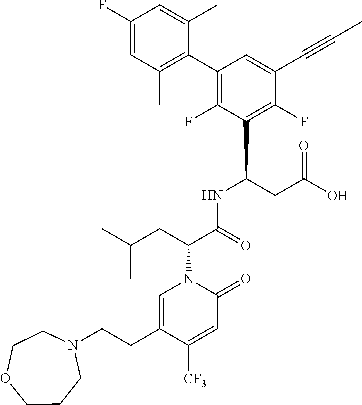 [SMILES: CC#Cc1cc(-c2c(C)cc(F)cc2C)c(F)c([C@@H](CC(=O)O)NC(=O)[C@@H](CC(C)C)n2cc(CCN3CCCOCC3)c(C(F)(F)F)cc2=O)c1F] |
| 14 | 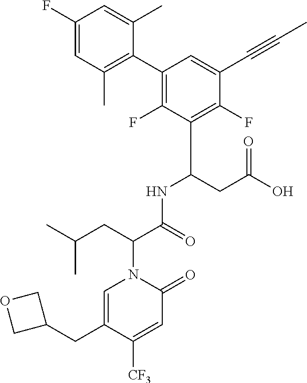 [SMILES: CC#Cc1cc(-c2c(C)cc(F)cc2C)c(F)c(C(CC(=O)O)NC(=O)C(CC(C)C)n2cc(CC3COC3)c(C(F)(F)F)cc2=O)c1F] |
| | 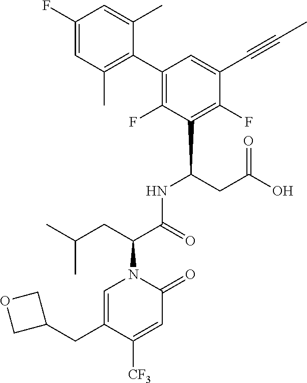 [SMILES: CC#Cc1cc(-c2c(C)cc(F)cc2C)c(F)c([C@@H](CC(=O)O)NC(=O)[C@H](CC(C)C)n2cc(CC3COC3)c(C(F)(F)F)cc2=O)c1F] |
| | 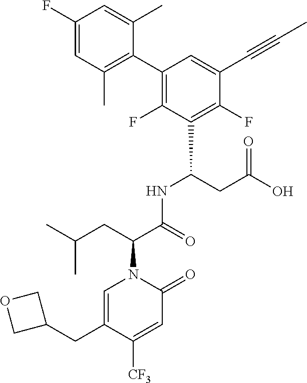 [SMILES: CC#Cc1cc(-c2c(C)cc(F)cc2C)c(F)c([C@H](CC(=O)O)NC(=O)[C@H](CC(C)C)n2cc(CC3COC3)c(C(F)(F)F)cc2=O)c1F] |
| | 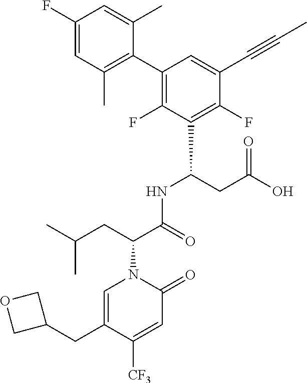 [SMILES: CC#Cc1cc(-c2c(C)cc(F)cc2C)c(F)c([C@H](CC(=O)O)NC(=O)[C@@H](CC(C)C)n2cc(CC3COC3)c(C(F)(F)F)cc2=O)c1F] |
| | 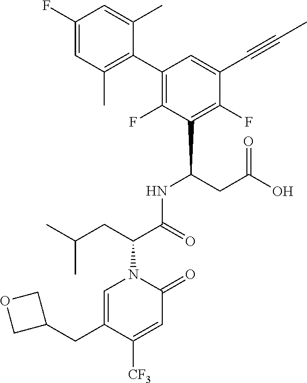 [SMILES: CC#Cc1cc(-c2c(C)cc(F)cc2C)c(F)c([C@@H](CC(=O)O)NC(=O)[C@@H](CC(C)C)n2cc(CC3COC3)c(C(F)(F)F)cc2=O)c1F] |
| 15 | 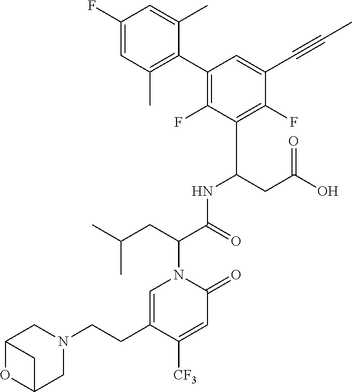 [SMILES: CC#Cc1cc(-c2c(C)cc(F)cc2C)c(F)c(C(CC(=O)O)NC(=O)C(CC(C)C)n2cc(CCN3CC4CC(C3)O4)c(C(F)(F)F)cc2=O)c1F] |
| | 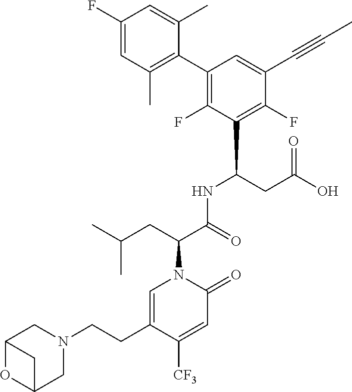 [SMILES: CC#Cc1cc(-c2c(C)cc(F)cc2C)c(F)c([C@@H](CC(=O)O)NC(=O)[C@H](CC(C)C)n2cc(CCN3CC4CC(C3)O4)c(C(F)(F)F)cc2=O)c1F] |
| | 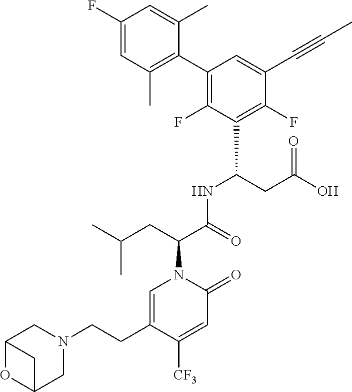 [SMILES: CC#Cc1cc(-c2c(C)cc(F)cc2C)c(F)c([C@H](CC(=O)O)NC(=O)[C@H](CC(C)C)n2cc(CCN3CC4CC(C3)O4)c(C(F)(F)F)cc2=O)c1F] |
| | 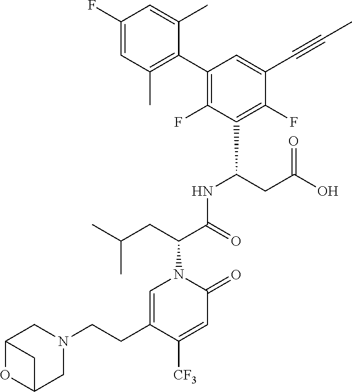 [SMILES: CC#Cc1cc(-c2c(C)cc(F)cc2C)c(F)c([C@H](CC(=O)O)NC(=O)[C@@H](CC(C)C)n2cc(CCN3CC4CC(C3)O4)c(C(F)(F)F)cc2=O)c1F] |
| | 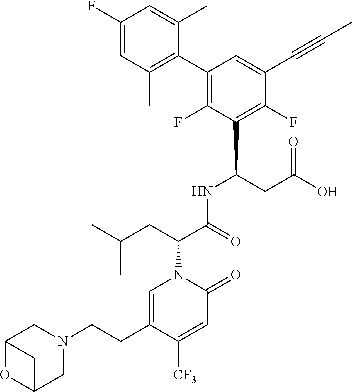 [SMILES: CC#Cc1cc(-c2c(C)cc(F)cc2C)c(F)c([C@@H](CC(=O)O)NC(=O)[C@@H](CC(C)C)n2cc(CCN3CC4CC(C3)O4)c(C(F)(F)F)cc2=O)c1F] |
| 16 | 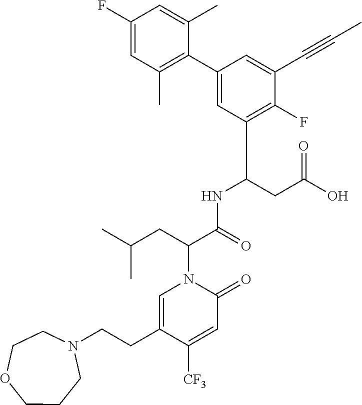 [SMILES: CC#Cc1cc(-c2c(C)cc(F)cc2C)cc(C(CC(=O)O)NC(=O)C(CC(C)C)n2cc(CCN3CCCOCC3)c(C(F)(F)F)cc2=O)c1F] |
| | 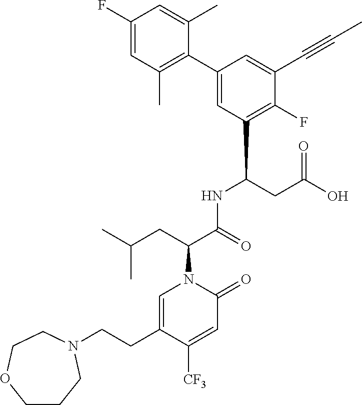 [SMILES: CC#Cc1cc(-c2c(C)cc(F)cc2C)cc([C@@H](CC(=O)O)NC(=O)[C@H](CC(C)C)n2cc(CCN3CCCOCC3)c(C(F)(F)F)cc2=O)c1F] |
| | 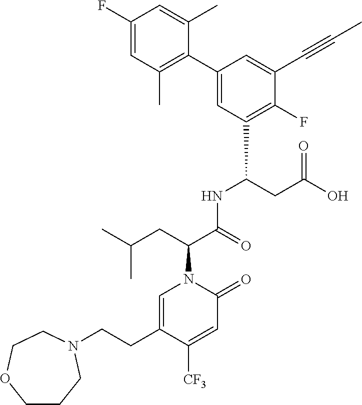 [SMILES: CC#Cc1cc(-c2c(C)cc(F)cc2C)cc([C@H](CC(=O)O)NC(=O)[C@H](CC(C)C)n2cc(CCN3CCCOCC3)c(C(F)(F)F)cc2=O)c1F] |
| | 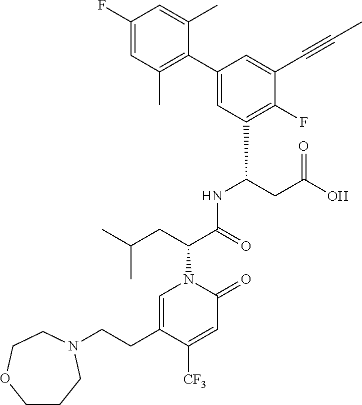 [SMILES: CC#Cc1cc(-c2c(C)cc(F)cc2C)cc([C@H](CC(=O)O)NC(=O)[C@@H](CC(C)C)n2cc(CCN3CCCOCC3)c(C(F)(F)F)cc2=O)c1F] |
| | 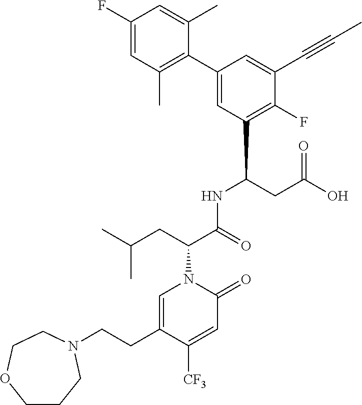 [SMILES: CC#Cc1cc(-c2c(C)cc(F)cc2C)cc([C@@H](CC(=O)O)NC(=O)[C@@H](CC(C)C)n2cc(CCN3CCCOCC3)c(C(F)(F)F)cc2=O)c1F] |
| 17 | 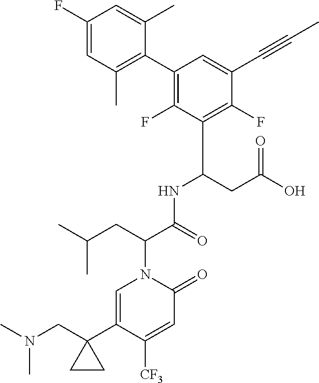 [SMILES: CC#Cc1cc(-c2c(C)cc(F)cc2C)c(F)c(C(CC(=O)O)NC(=O)C(CC(C)C)n2cc(C3(CN(C)C)CC3)c(C(F)(F)F)cc2=O)c1F] |
| | 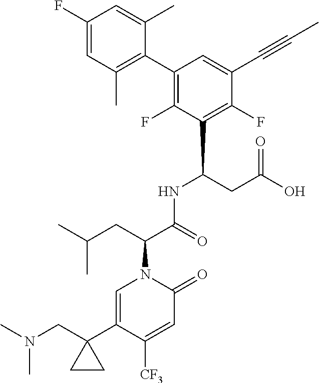 [SMILES: CC#Cc1cc(-c2c(C)cc(F)cc2C)c(F)c([C@@H](CC(=O)O)NC(=O)[C@H](CC(C)C)n2cc(C3(CN(C)C)CC3)c(C(F)(F)F)cc2=O)c1F] |
| | 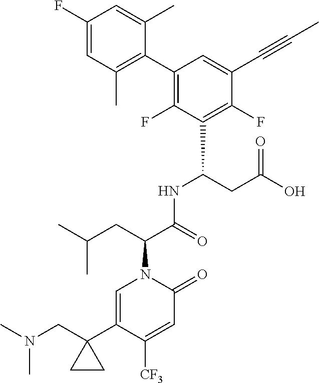 [SMILES: CC#Cc1cc(-c2c(C)cc(F)cc2C)c(F)c([C@H](CC(=O)O)NC(=O)[C@H](CC(C)C)n2cc(C3(CN(C)C)CC3)c(C(F)(F)F)cc2=O)c1F] |
| | 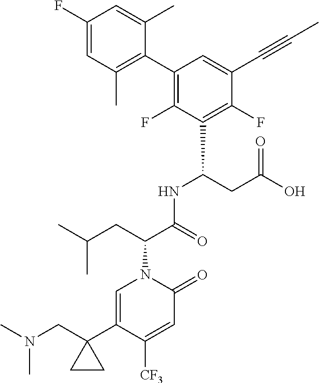 [SMILES: CC#Cc1cc(-c2c(C)cc(F)cc2C)c(F)c([C@H](CC(=O)O)NC(=O)[C@@H](CC(C)C)n2cc(C3(CN(C)C)CC3)c(C(F)(F)F)cc2=O)c1F] |
| | 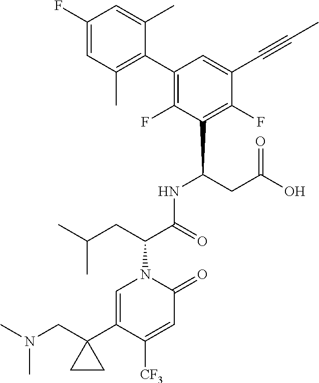 [SMILES: CC#Cc1cc(-c2c(C)cc(F)cc2C)c(F)c([C@@H](CC(=O)O)NC(=O)[C@@H](CC(C)C)n2cc(C3(CN(C)C)CC3)c(C(F)(F)F)cc2=O)c1F] |
| 18 | 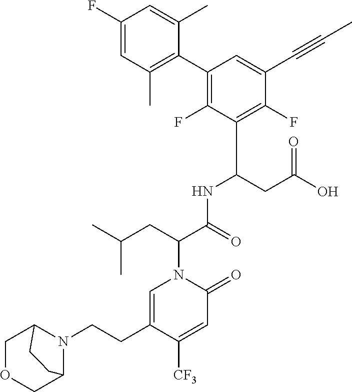 [SMILES: CC#Cc1cc(-c2c(C)cc(F)cc2C)c(F)c(C(CC(=O)O)NC(=O)C(CC(C)C)n2cc(CCN3C4CCC3COC4)c(C(F)(F)F)cc2=O)c1F] |
| | 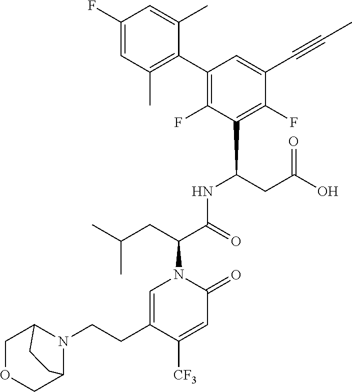 [SMILES: CC#Cc1cc(-c2c(C)cc(F)cc2C)c(F)c([C@@H](CC(=O)O)NC(=O)[C@H](CC(C)C)n2cc(CCN3C4CCC3COC4)c(C(F)(F)F)cc2=O)c1F] |
| | 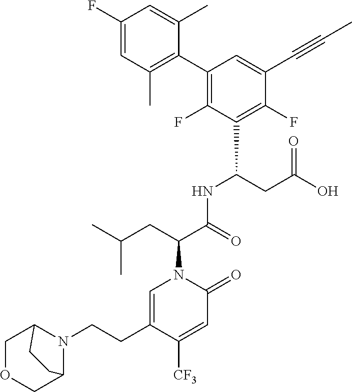 [SMILES: CC#Cc1cc(-c2c(C)cc(F)cc2C)c(F)c([C@H](CC(=O)O)NC(=O)[C@H](CC(C)C)n2cc(CCN3C4CCC3COC4)c(C(F)(F)F)cc2=O)c1F] |
| | 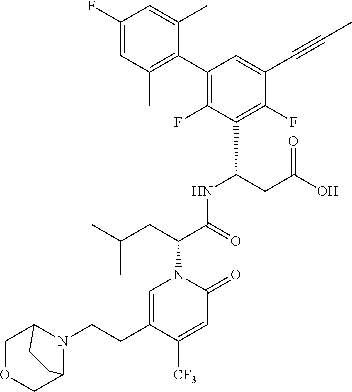 [SMILES: CC#Cc1cc(-c2c(C)cc(F)cc2C)c(F)c([C@H](CC(=O)O)NC(=O)[C@@H](CC(C)C)n2cc(CCN3C4CCC3COC4)c(C(F)(F)F)cc2=O)c1F] |
| | 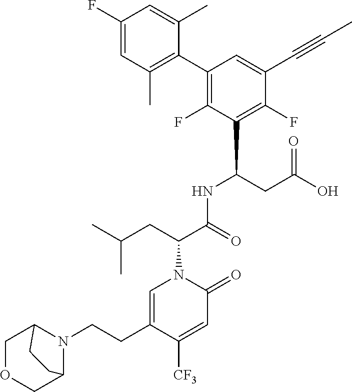 [SMILES: CC#Cc1cc(-c2c(C)cc(F)cc2C)c(F)c([C@@H](CC(=O)O)NC(=O)[C@@H](CC(C)C)n2cc(CCN3C4CCC3COC4)c(C(F)(F)F)cc2=O)c1F] |
| 19 | 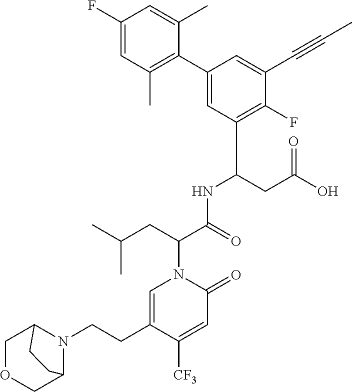 [SMILES: CC#Cc1cc(-c2c(C)cc(F)cc2C)cc(C(CC(=O)O)NC(=O)C(CC(C)C)n2cc(CCN3C4CCC3COC4)c(C(F)(F)F)cc2=O)c1F] |
| | 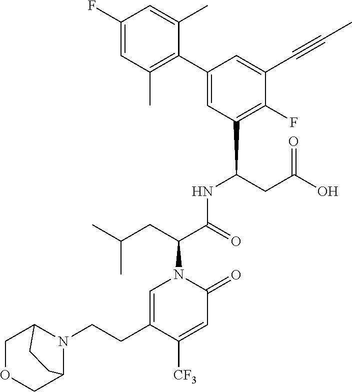 [SMILES: CC#Cc1cc(-c2c(C)cc(F)cc2C)cc([C@@H](CC(=O)O)NC(=O)[C@H](CC(C)C)n2cc(CCN3C4CCC3COC4)c(C(F)(F)F)cc2=O)c1F] |
| | 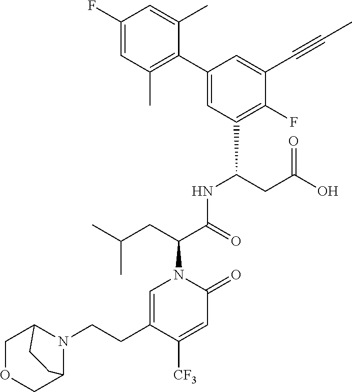 [SMILES: CC#Cc1cc(-c2c(C)cc(F)cc2C)cc([C@H](CC(=O)O)NC(=O)[C@H](CC(C)C)n2cc(CCN3C4CCC3COC4)c(C(F)(F)F)cc2=O)c1F] |
| | 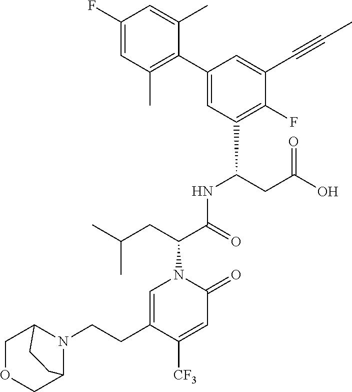 [SMILES: CC#Cc1cc(-c2c(C)cc(F)cc2C)cc([C@H](CC(=O)O)NC(=O)[C@@H](CC(C)C)n2cc(CCN3C4CCC3COC4)c(C(F)(F)F)cc2=O)c1F] |
| | 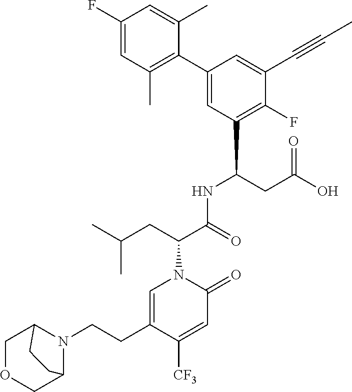 [SMILES: CC#Cc1cc(-c2c(C)cc(F)cc2C)cc([C@@H](CC(=O)O)NC(=O)[C@@H](CC(C)C)n2cc(CCN3C4CCC3COC4)c(C(F)(F)F)cc2=O)c1F] |
| 20 | 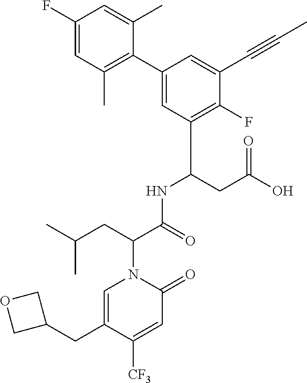 [SMILES: CC#Cc1cc(-c2c(C)cc(F)cc2C)cc(C(CC(=O)O)NC(=O)C(CC(C)C)n2cc(CC3COC3)c(C(F)(F)F)cc2=O)c1F] |
| | 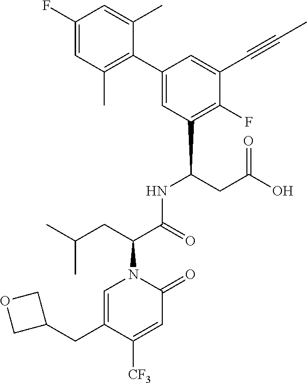 [SMILES: CC#Cc1cc(-c2c(C)cc(F)cc2C)cc([C@@H](CC(=O)O)NC(=O)[C@H](CC(C)C)n2cc(CC3COC3)c(C(F)(F)F)cc2=O)c1F] |
| | 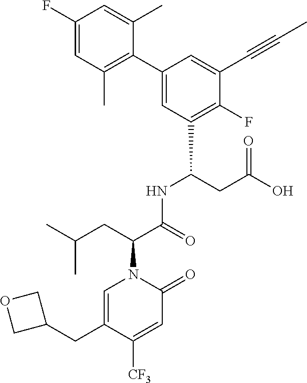 [SMILES: CC#Cc1cc(-c2c(C)cc(F)cc2C)cc([C@H](CC(=O)O)NC(=O)[C@H](CC(C)C)n2cc(CC3COC3)c(C(F)(F)F)cc2=O)c1F] |
| | 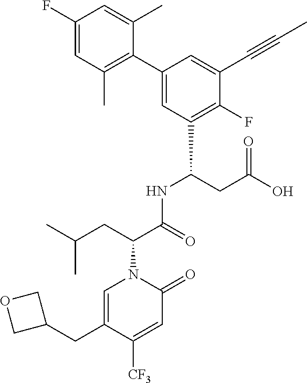 [SMILES: CC#Cc1cc(-c2c(C)cc(F)cc2C)cc([C@H](CC(=O)O)NC(=O)[C@@H](CC(C)C)n2cc(CC3COC3)c(C(F)(F)F)cc2=O)c1F] |
| | 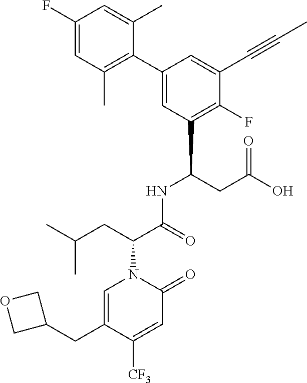 [SMILES: CC#Cc1cc(-c2c(C)cc(F)cc2C)cc([C@@H](CC(=O)O)NC(=O)[C@@H](CC(C)C)n2cc(CC3COC3)c(C(F)(F)F)cc2=O)c1F] |
| 21 | 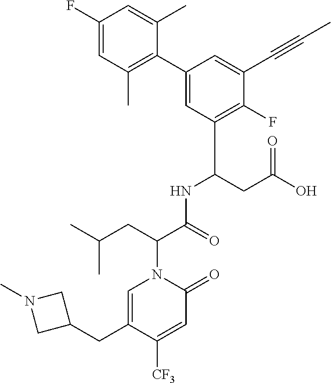 [SMILES: CC#Cc1cc(-c2c(C)cc(F)cc2C)cc(C(CC(=O)O)NC(=O)C(CC(C)C)n2cc(CC3CN(C)C3)c(C(F)(F)F)cc2=O)c1F] |
| | 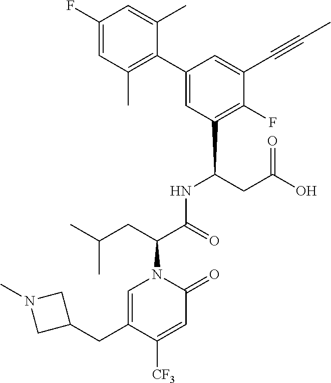 [SMILES: CC#Cc1cc(-c2c(C)cc(F)cc2C)cc([C@@H](CC(=O)O)NC(=O)[C@H](CC(C)C)n2cc(CC3CN(C)C3)c(C(F)(F)F)cc2=O)c1F] |
| | 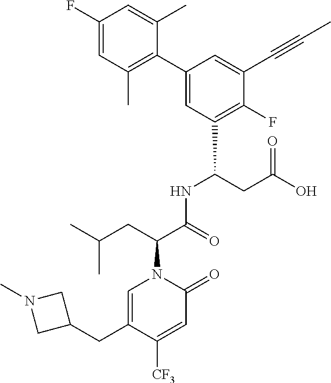 [SMILES: CC#Cc1cc(-c2c(C)cc(F)cc2C)cc([C@H](CC(=O)O)NC(=O)[C@H](CC(C)C)n2cc(CC3CN(C)C3)c(C(F)(F)F)cc2=O)c1F] |
| | 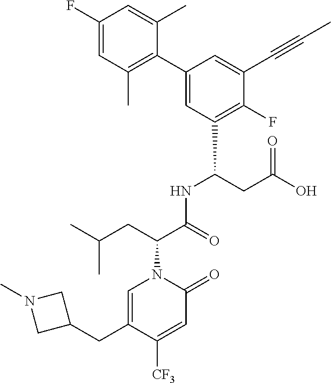 [SMILES: CC#Cc1cc(-c2c(C)cc(F)cc2C)cc([C@H](CC(=O)O)NC(=O)[C@@H](CC(C)C)n2cc(CC3CN(C)C3)c(C(F)(F)F)cc2=O)c1F] |
| | 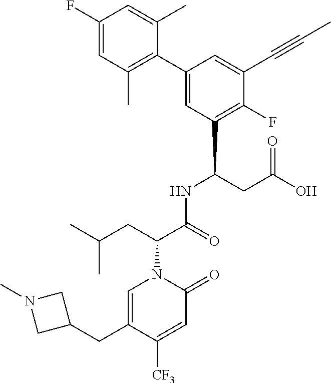 [SMILES: CC#Cc1cc(-c2c(C)cc(F)cc2C)cc([C@@H](CC(=O)O)NC(=O)[C@@H](CC(C)C)n2cc(CC3CN(C)C3)c(C(F)(F)F)cc2=O)c1F] |
| 22 | 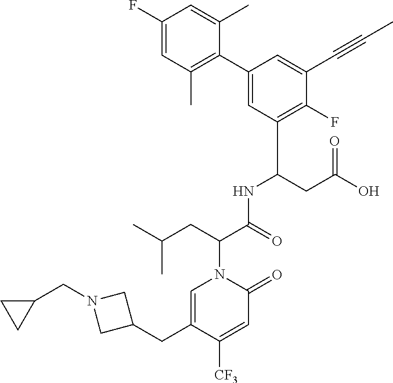 [SMILES: CC#Cc1cc(-c2c(C)cc(F)cc2C)cc(C(CC(=O)O)NC(=O)C(CC(C)C)n2cc(CC3CN(CC4CC4)C3)c(C(F)(F)F)cc2=O)c1F] |
| | 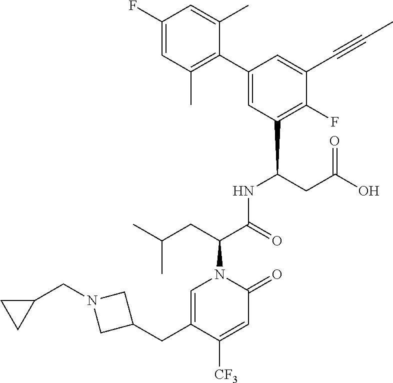 [SMILES: CC#Cc1cc(-c2c(C)cc(F)cc2C)cc([C@@H](CC(=O)O)NC(=O)[C@H](CC(C)C)n2cc(CC3CN(CC4CC4)C3)c(C(F)(F)F)cc2=O)c1F] |
| | 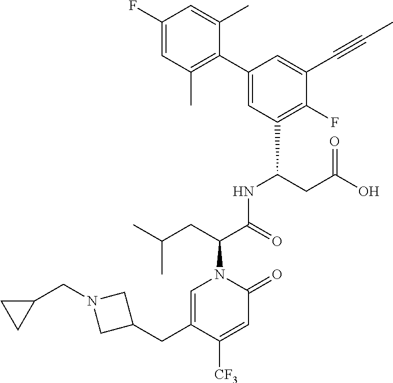 [SMILES: CC#Cc1cc(-c2c(C)cc(F)cc2C)cc([C@H](CC(=O)O)NC(=O)[C@H](CC(C)C)n2cc(CC3CN(CC4CC4)C3)c(C(F)(F)F)cc2=O)c1F] |
| | 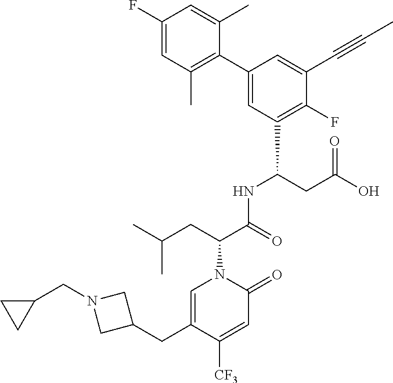 [SMILES: CC#Cc1cc(-c2c(C)cc(F)cc2C)cc([C@H](CC(=O)O)NC(=O)[C@@H](CC(C)C)n2cc(CC3CN(CC4CC4)C3)c(C(F)(F)F)cc2=O)c1F] |
| | 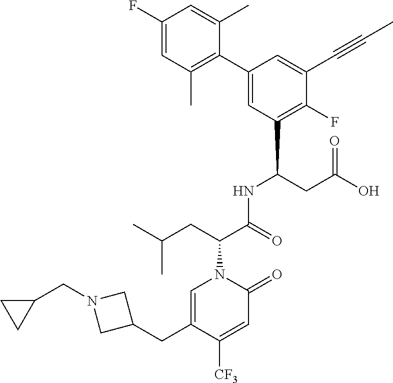 [SMILES: CC#Cc1cc(-c2c(C)cc(F)cc2C)cc([C@@H](CC(=O)O)NC(=O)[C@@H](CC(C)C)n2cc(CC3CN(CC4CC4)C3)c(C(F)(F)F)cc2=O)c1F] |
| 23 | 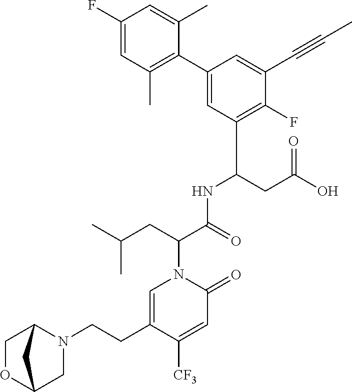 [SMILES: CC#Cc1cc(-c2c(C)cc(F)cc2C)cc(C(CC(=O)O)NC(=O)C(CC(C)C)n2cc(CCN3C[C@H]4C[C@@H]3CO4)c(C(F)(F)F)cc2=O)c1F] |
| | 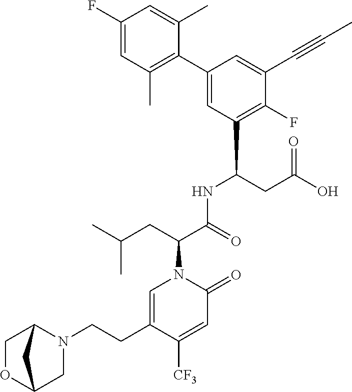 [SMILES: CC#Cc1cc(-c2c(C)cc(F)cc2C)cc([C@@H](CC(=O)O)NC(=O)[C@H](CC(C)C)n2cc(CCN3C[C@H]4C[C@@H]3CO4)c(C(F)(F)F)cc2=O)c1F] |
| | 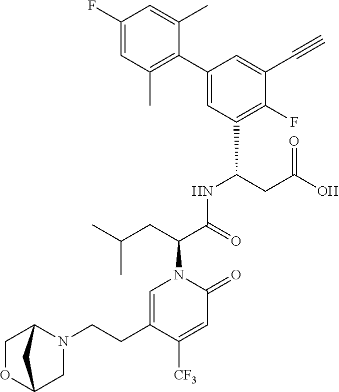 [SMILES: C#Cc1cc(-c2c(C)cc(F)cc2C)cc([C@H](CC(=O)O)NC(=O)[C@H](CC(C)C)n2cc(CCN3C[C@H]4C[C@@H]3CO4)c(C(F)(F)F)cc2=O)c1F] |
| | 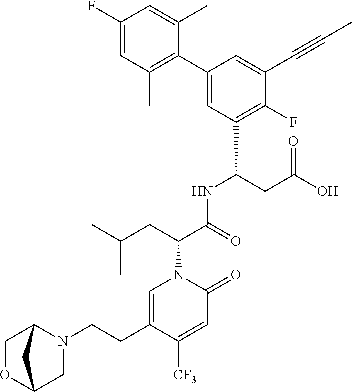 [SMILES: CC#Cc1cc(-c2c(C)cc(F)cc2C)cc([C@H](CC(=O)O)NC(=O)[C@@H](CC(C)C)n2cc(CCN3C[C@H]4C[C@@H]3CO4)c(C(F)(F)F)cc2=O)c1F] |
| | 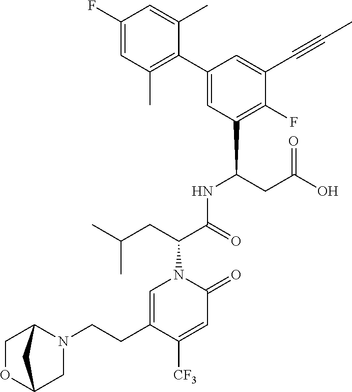 [SMILES: CC#Cc1cc(-c2c(C)cc(F)cc2C)cc([C@@H](CC(=O)O)NC(=O)[C@@H](CC(C)C)n2cc(CCN3C[C@H]4C[C@@H]3CO4)c(C(F)(F)F)cc2=O)c1F] |
| 24 | 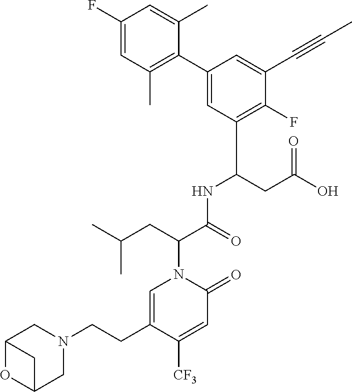 [SMILES: CC#Cc1cc(-c2c(C)cc(F)cc2C)cc(C(CC(=O)O)NC(=O)C(CC(C)C)n2cc(CCN3CC4CC(C3)O4)c(C(F)(F)F)cc2=O)c1F] |
| | 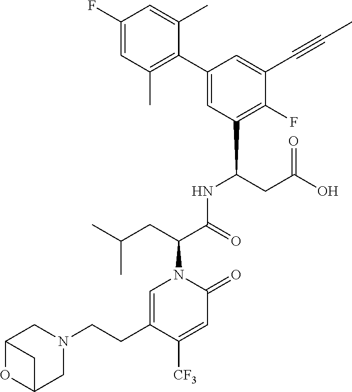 [SMILES: CC#Cc1cc(-c2c(C)cc(F)cc2C)cc([C@@H](CC(=O)O)NC(=O)[C@H](CC(C)C)n2cc(CCN3CC4CC(C3)O4)c(C(F)(F)F)cc2=O)c1F] |
| | 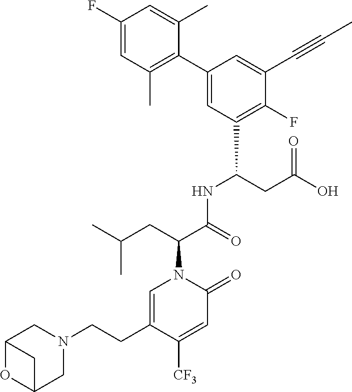 [SMILES: CC#Cc1cc(-c2c(C)cc(F)cc2C)cc([C@H](CC(=O)O)NC(=O)[C@H](CC(C)C)n2cc(CCN3CC4CC(C3)O4)c(C(F)(F)F)cc2=O)c1F] |
| | 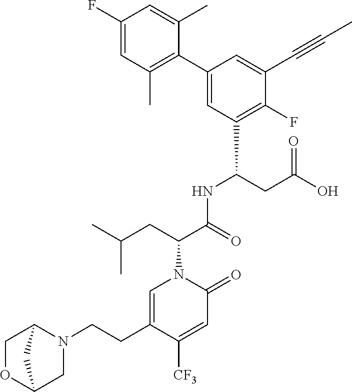 [SMILES: CC#Cc1cc(-c2c(C)cc(F)cc2C)cc([C@H](CC(=O)O)NC(=O)[C@@H](CC(C)C)n2cc(CCN3C[C@@H]4C[C@H]3CO4)c(C(F)(F)F)cc2=O)c1F] |
| | 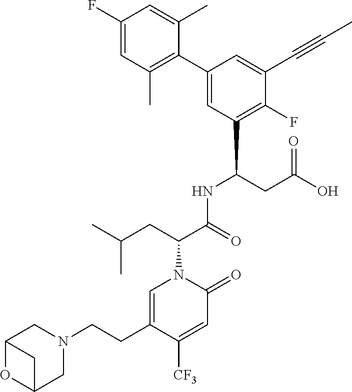 [SMILES: CC#Cc1cc(-c2c(C)cc(F)cc2C)cc([C@@H](CC(=O)O)NC(=O)[C@@H](CC(C)C)n2cc(CCN3CC4CC(C3)O4)c(C(F)(F)F)cc2=O)c1F] |
| 25 | 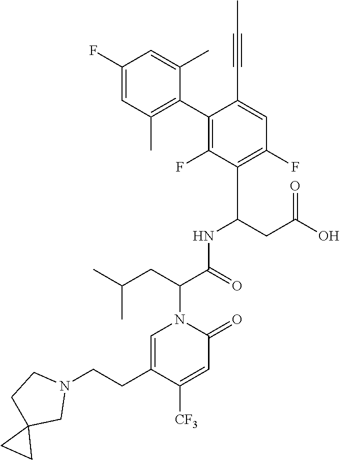 [SMILES: CC#Cc1cc(F)c(C(CC(=O)O)NC(=O)C(CC(C)C)n2cc(CCN3CCC4(CC4)C3)c(C(F)(F)F)cc2=O)c(F)c1-c1c(C)cc(F)cc1C] |
| | 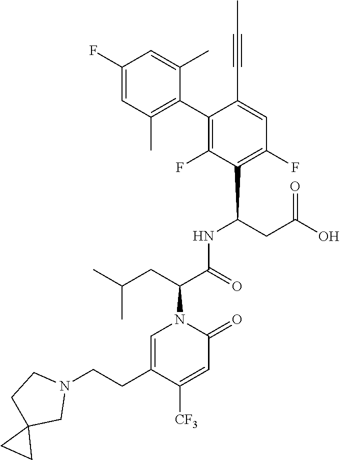 [SMILES: CC#Cc1cc(F)c([C@@H](CC(=O)O)NC(=O)[C@H](CC(C)C)n2cc(CCN3CCC4(CC4)C3)c(C(F)(F)F)cc2=O)c(F)c1-c1c(C)cc(F)cc1C] |
| | 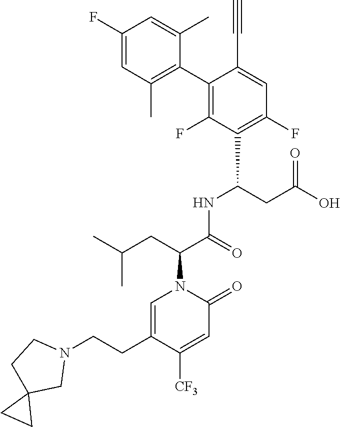 [SMILES: C#Cc1cc(F)c([C@H](CC(=O)O)NC(=O)[C@H](CC(C)C)n2cc(CCN3CCC4(CC4)C3)c(C(F)(F)F)cc2=O)c(F)c1-c1c(C)cc(F)cc1C] |
| | 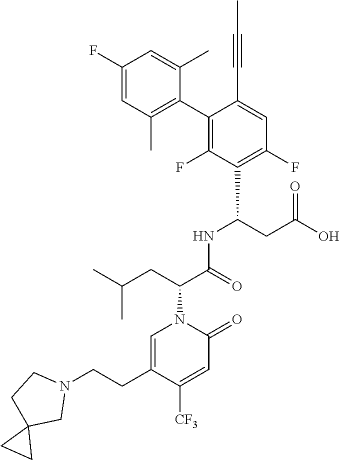 [SMILES: CC#Cc1cc(F)c([C@H](CC(=O)O)NC(=O)[C@@H](CC(C)C)n2cc(CCN3CCC4(CC4)C3)c(C(F)(F)F)cc2=O)c(F)c1-c1c(C)cc(F)cc1C] |
| | 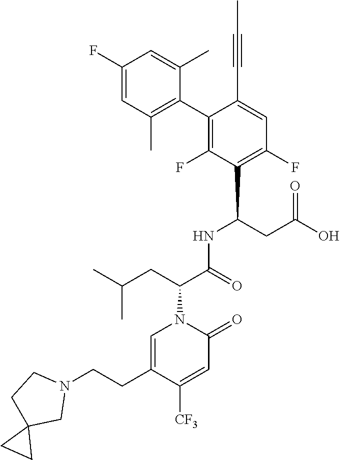 [SMILES: CC#Cc1cc(F)c([C@@H](CC(=O)O)NC(=O)[C@@H](CC(C)C)n2cc(CCN3CCC4(CC4)C3)c(C(F)(F)F)cc2=O)c(F)c1-c1c(C)cc(F)cc1C] |
| 26 | 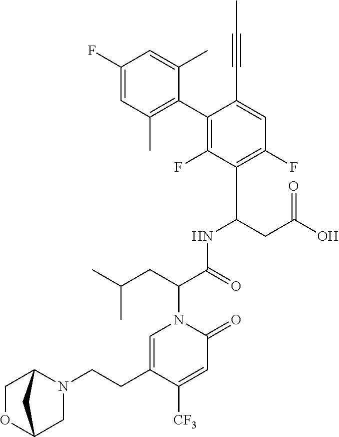 [SMILES: CC#Cc1cc(F)c(C(CC(=O)O)NC(=O)C(CC(C)C)n2cc(CCN3C[C@H]4C[C@@H]3CO4)c(C(F)(F)F)cc2=O)c(F)c1-c1c(C)cc(F)cc1C] |
| | 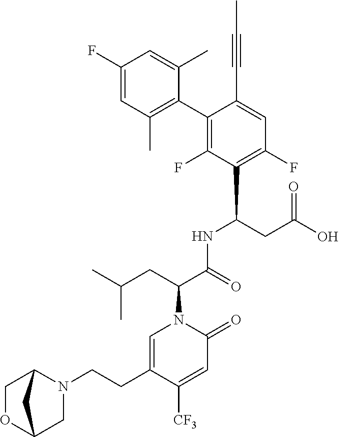 [SMILES: CC#Cc1cc(F)c([C@@H](CC(=O)O)NC(=O)[C@H](CC(C)C)n2cc(CCN3C[C@H]4C[C@@H]3CO4)c(C(F)(F)F)cc2=O)c(F)c1-c1c(C)cc(F)cc1C] |
| | 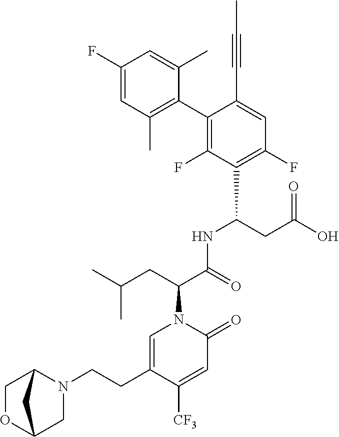 [SMILES: CC#Cc1cc(F)c([C@H](CC(=O)O)NC(=O)[C@H](CC(C)C)n2cc(CCN3C[C@H]4C[C@@H]3CO4)c(C(F)(F)F)cc2=O)c(F)c1-c1c(C)cc(F)cc1C] |
| | 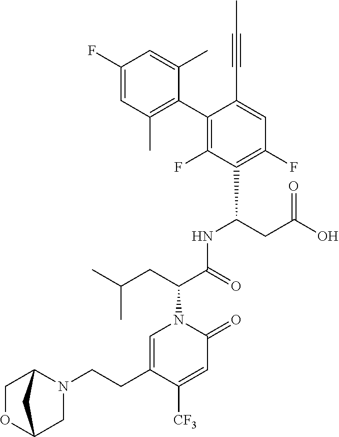 [SMILES: CC#Cc1cc(F)c([C@H](CC(=O)O)NC(=O)[C@@H](CC(C)C)n2cc(CCN3C[C@H]4C[C@@H]3CO4)c(C(F)(F)F)cc2=O)c(F)c1-c1c(C)cc(F)cc1C] |
| | 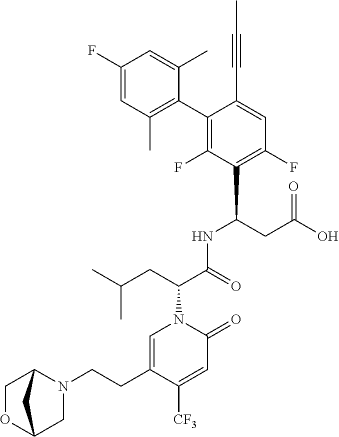 [SMILES: CC#Cc1cc(F)c([C@@H](CC(=O)O)NC(=O)[C@@H](CC(C)C)n2cc(CCN3C[C@H]4C[C@@H]3CO4)c(C(F)(F)F)cc2=O)c(F)c1-c1c(C)cc(F)cc1C] |
| 27 | 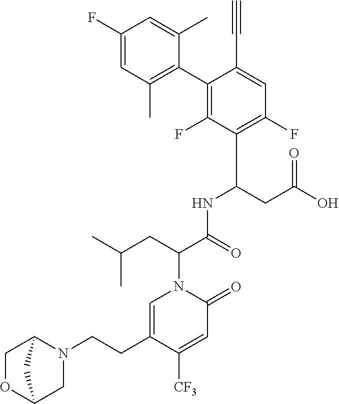 [SMILES: C#Cc1cc(F)c(C(CC(=O)O)NC(=O)C(CC(C)C)n2cc(CCN3C[C@@H]4C[C@H]3CO4)c(C(F)(F)F)cc2=O)c(F)c1-c1c(C)cc(F)cc1C] |
| | 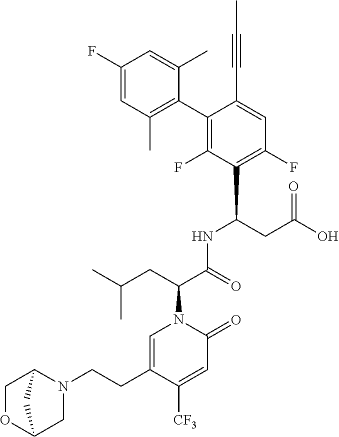 [SMILES: CC#Cc1cc(F)c([C@@H](CC(=O)O)NC(=O)[C@H](CC(C)C)n2cc(CCN3C[C@@H]4C[C@H]3CO4)c(C(F)(F)F)cc2=O)c(F)c1-c1c(C)cc(F)cc1C] |
| | 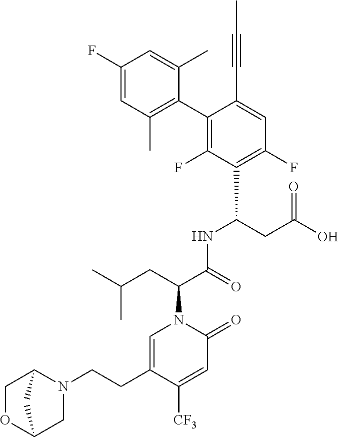 [SMILES: CC#Cc1cc(F)c([C@H](CC(=O)O)NC(=O)[C@H](CC(C)C)n2cc(CCN3C[C@@H]4C[C@H]3CO4)c(C(F)(F)F)cc2=O)c(F)c1-c1c(C)cc(F)cc1C] |
| | 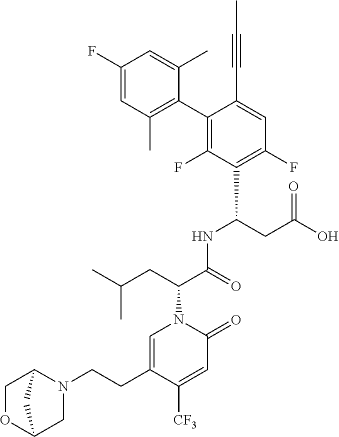 [SMILES: CC#Cc1cc(F)c([C@H](CC(=O)O)NC(=O)[C@@H](CC(C)C)n2cc(CCN3C[C@@H]4C[C@H]3CO4)c(C(F)(F)F)cc2=O)c(F)c1-c1c(C)cc(F)cc1C] |
| | 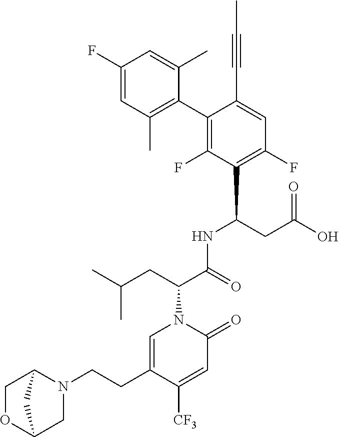 [SMILES: CC#Cc1cc(F)c([C@@H](CC(=O)O)NC(=O)[C@@H](CC(C)C)n2cc(CCN3C[C@@H]4C[C@H]3CO4)c(C(F)(F)F)cc2=O)c(F)c1-c1c(C)cc(F)cc1C] |
| 28 | 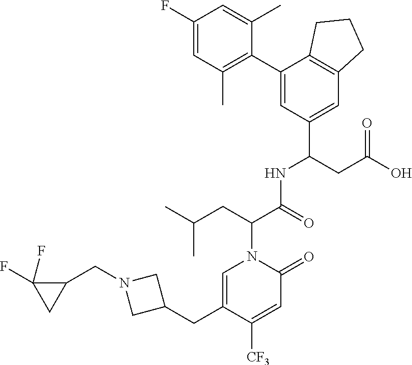 [SMILES: Cc1cc(F)cc(C)c1-c1cc(C(CC(=O)O)NC(=O)C(CC(C)C)n2cc(CC3CN(CC4CC4(F)F)C3)c(C(F)(F)F)cc2=O)cc2c1CCC2] |
| | 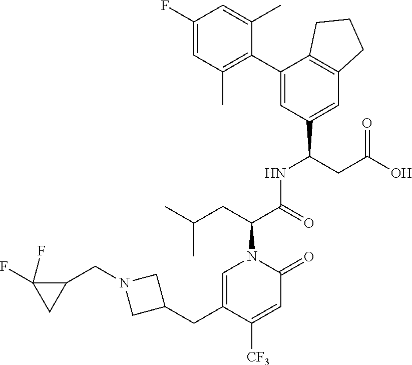 [SMILES: Cc1cc(F)cc(C)c1-c1cc([C@@H](CC(=O)O)NC(=O)[C@H](CC(C)C)n2cc(CC3CN(CC4CC4(F)F)C3)c(C(F)(F)F)cc2=O)cc2c1CCC2] |
| | 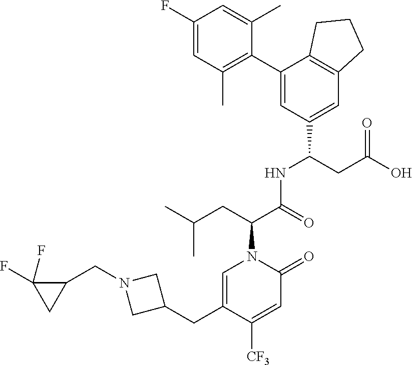 [SMILES: Cc1cc(F)cc(C)c1-c1cc([C@H](CC(=O)O)NC(=O)[C@H](CC(C)C)n2cc(CC3CN(CC4CC4(F)F)C3)c(C(F)(F)F)cc2=O)cc2c1CCC2] |
| | 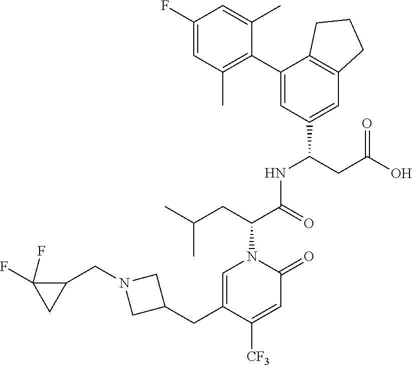 [SMILES: Cc1cc(F)cc(C)c1-c1cc([C@H](CC(=O)O)NC(=O)[C@@H](CC(C)C)n2cc(CC3CN(CC4CC4(F)F)C3)c(C(F)(F)F)cc2=O)cc2c1CCC2] |
| | 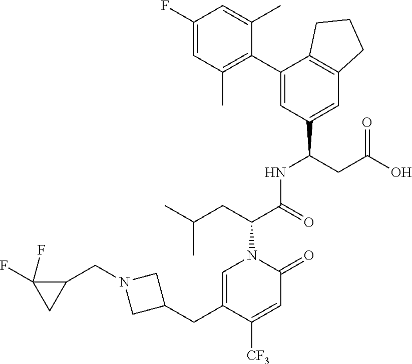 [SMILES: Cc1cc(F)cc(C)c1-c1cc([C@@H](CC(=O)O)NC(=O)[C@@H](CC(C)C)n2cc(CC3CN(CC4CC4(F)F)C3)c(C(F)(F)F)cc2=O)cc2c1CCC2] |
| 29 | 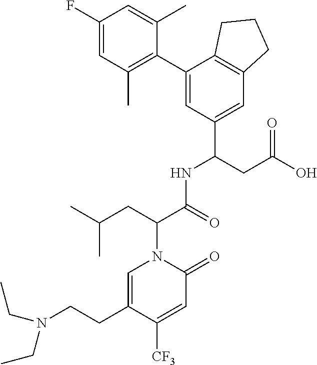 [SMILES: CCN(CC)CCc1cn(C(CC(C)C)C(=O)NC(CC(=O)O)c2cc3c(c(-c4c(C)cc(F)cc4C)c2)CCC3)c(=O)cc1C(F)(F)F] |
| | 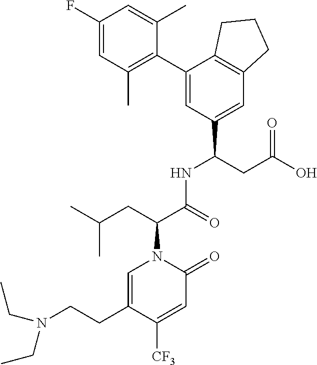 [SMILES: CCN(CC)CCc1cn([C@@H](CC(C)C)C(=O)N[C@H](CC(=O)O)c2cc3c(c(-c4c(C)cc(F)cc4C)c2)CCC3)c(=O)cc1C(F)(F)F] |
| | 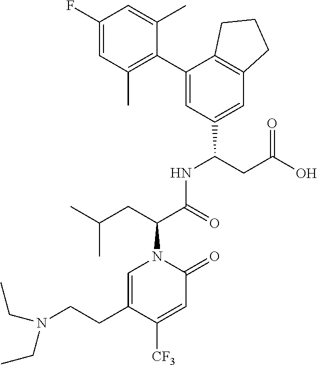 [SMILES: CCN(CC)CCc1cn([C@@H](CC(C)C)C(=O)N[C@@H](CC(=O)O)c2cc3c(c(-c4c(C)cc(F)cc4C)c2)CCC3)c(=O)cc1C(F)(F)F] |
| | 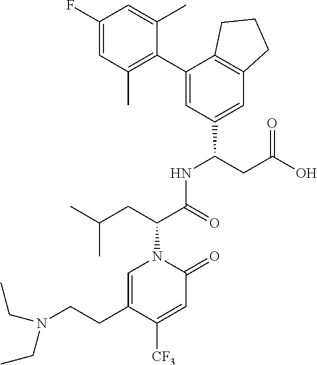 [SMILES: CCN(CC)CCc1cn([C@H](CC(C)C)C(=O)N[C@@H](CC(=O)O)c2cc3c(c(-c4c(C)cc(F)cc4C)c2)CCC3)c(=O)cc1C(F)(F)F] |
| | 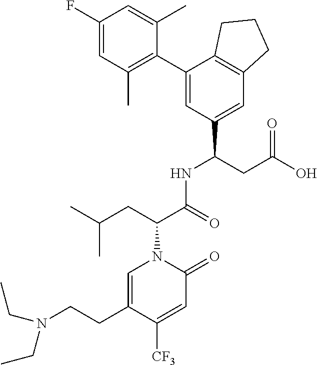 [SMILES: CCN(CC)CCc1cn([C@H](CC(C)C)C(=O)N[C@H](CC(=O)O)c2cc3c(c(-c4c(C)cc(F)cc4C)c2)CCC3)c(=O)cc1C(F)(F)F] |
| 30 | 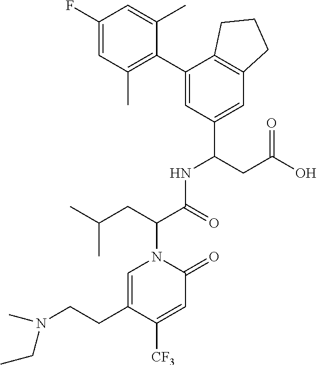 [SMILES: CCN(C)CCc1cn(C(CC(C)C)C(=O)NC(CC(=O)O)c2cc3c(c(-c4c(C)cc(F)cc4C)c2)CCC3)c(=O)cc1C(F)(F)F] |
| | 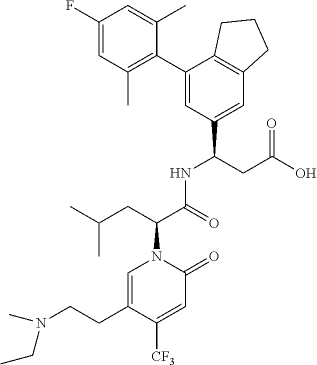 [SMILES: CCN(C)CCc1cn([C@@H](CC(C)C)C(=O)N[C@H](CC(=O)O)c2cc3c(c(-c4c(C)cc(F)cc4C)c2)CCC3)c(=O)cc1C(F)(F)F] |
| | 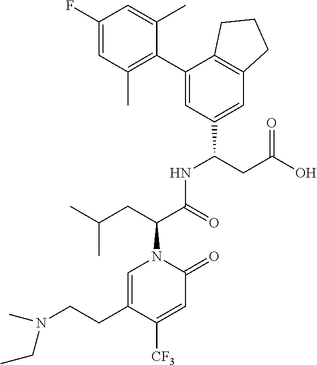 [SMILES: CCN(C)CCc1cn([C@@H](CC(C)C)C(=O)N[C@@H](CC(=O)O)c2cc3c(c(-c4c(C)cc(F)cc4C)c2)CCC3)c(=O)cc1C(F)(F)F] |
| | 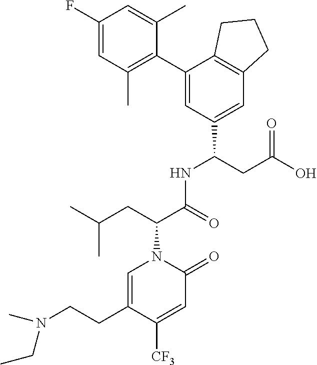 [SMILES: CCN(C)CCc1cn([C@H](CC(C)C)C(=O)N[C@@H](CC(=O)O)c2cc3c(c(-c4c(C)cc(F)cc4C)c2)CCC3)c(=O)cc1C(F)(F)F] |
| | 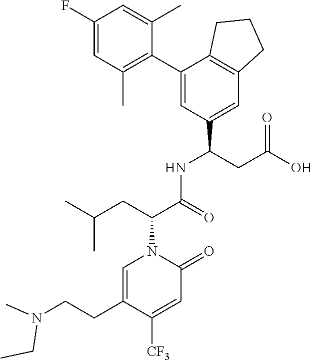 [SMILES: CCN(C)CCc1cn([C@H](CC(C)C)C(=O)N[C@H](CC(=O)O)c2cc3c(c(-c4c(C)cc(F)cc4C)c2)CCC3)c(=O)cc1C(F)(F)F] |
| 31 | 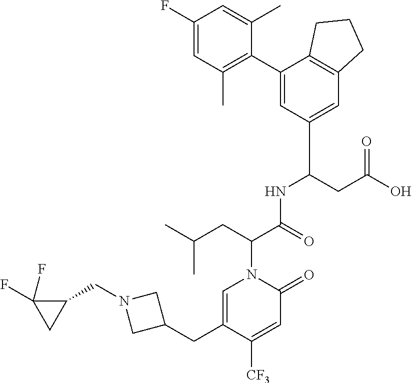 [SMILES: Cc1cc(F)cc(C)c1-c1cc(C(CC(=O)O)NC(=O)C(CC(C)C)n2cc(CC3CN(C[C@@H]4CC4(F)F)C3)c(C(F)(F)F)cc2=O)cc2c1CCC2] |
| | 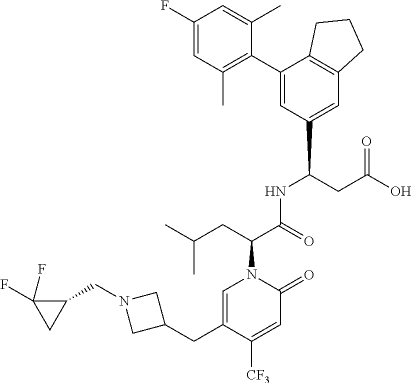 [SMILES: Cc1cc(F)cc(C)c1-c1cc([C@@H](CC(=O)O)NC(=O)[C@H](CC(C)C)n2cc(CC3CN(C[C@@H]4CC4(F)F)C3)c(C(F)(F)F)cc2=O)cc2c1CCC2] |
| | 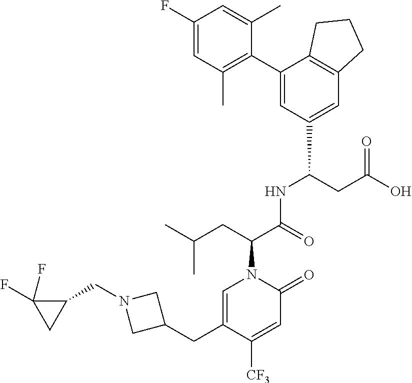 [SMILES: Cc1cc(F)cc(C)c1-c1cc([C@H](CC(=O)O)NC(=O)[C@H](CC(C)C)n2cc(CC3CN(C[C@@H]4CC4(F)F)C3)c(C(F)(F)F)cc2=O)cc2c1CCC2] |
| | 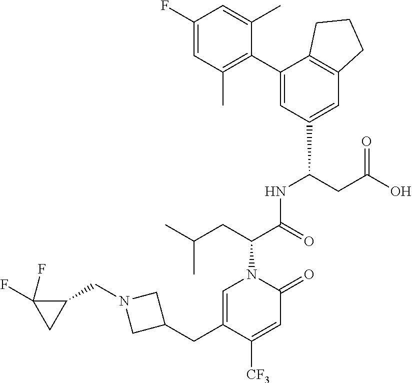 [SMILES: Cc1cc(F)cc(C)c1-c1cc([C@H](CC(=O)O)NC(=O)[C@@H](CC(C)C)n2cc(CC3CN(C[C@@H]4CC4(F)F)C3)c(C(F)(F)F)cc2=O)cc2c1CCC2] |
| | 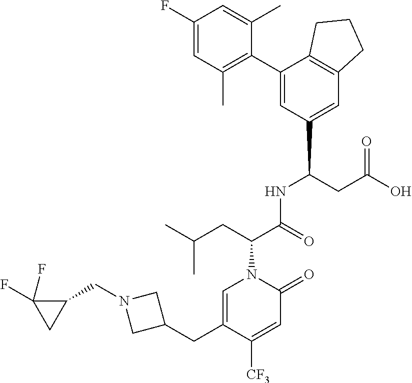 [SMILES: Cc1cc(F)cc(C)c1-c1cc([C@@H](CC(=O)O)NC(=O)[C@@H](CC(C)C)n2cc(CC3CN(C[C@@H]4CC4(F)F)C3)c(C(F)(F)F)cc2=O)cc2c1CCC2] |
| 32 | 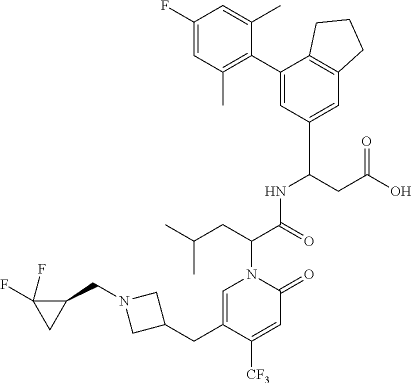 [SMILES: Cc1cc(F)cc(C)c1-c1cc(C(CC(=O)O)NC(=O)C(CC(C)C)n2cc(CC3CN(C[C@H]4CC4(F)F)C3)c(C(F)(F)F)cc2=O)cc2c1CCC2] |
| | 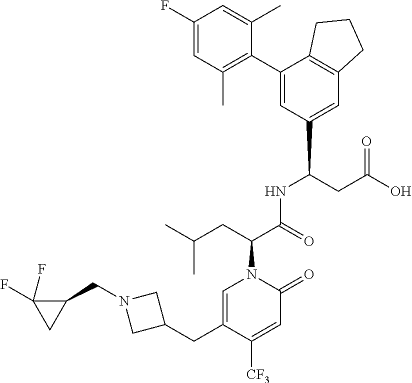 [SMILES: Cc1cc(F)cc(C)c1-c1cc([C@@H](CC(=O)O)NC(=O)[C@H](CC(C)C)n2cc(CC3CN(C[C@H]4CC4(F)F)C3)c(C(F)(F)F)cc2=O)cc2c1CCC2] |
| | 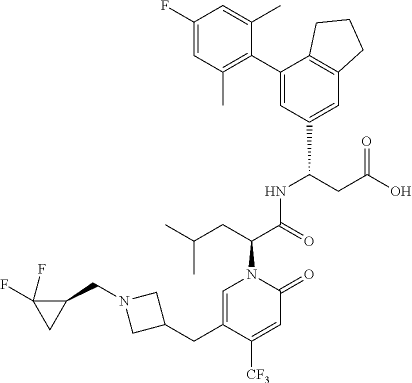 [SMILES: Cc1cc(F)cc(C)c1-c1cc([C@H](CC(=O)O)NC(=O)[C@H](CC(C)C)n2cc(CC3CN(C[C@H]4CC4(F)F)C3)c(C(F)(F)F)cc2=O)cc2c1CCC2] |
| | 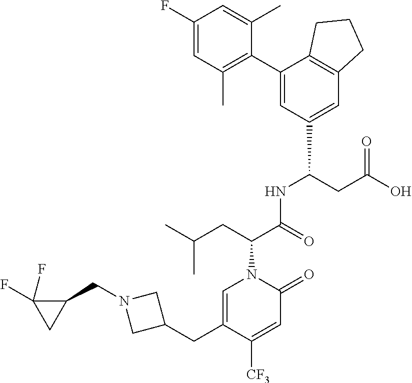 [SMILES: Cc1cc(F)cc(C)c1-c1cc([C@H](CC(=O)O)NC(=O)[C@@H](CC(C)C)n2cc(CC3CN(C[C@H]4CC4(F)F)C3)c(C(F)(F)F)cc2=O)cc2c1CCC2] |
| | 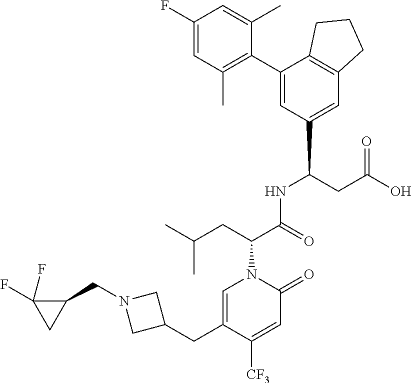 [SMILES: Cc1cc(F)cc(C)c1-c1cc([C@@H](CC(=O)O)NC(=O)[C@@H](CC(C)C)n2cc(CC3CN(C[C@H]4CC4(F)F)C3)c(C(F)(F)F)cc2=O)cc2c1CCC2] |
| 33 | 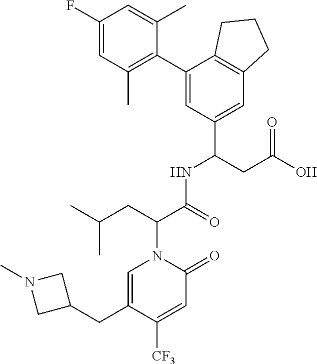 [SMILES: Cc1cc(F)cc(C)c1-c1cc(C(CC(=O)O)NC(=O)C(CC(C)C)n2cc(CC3CN(C)C3)c(C(F)(F)F)cc2=O)cc2c1CCC2] |
| | 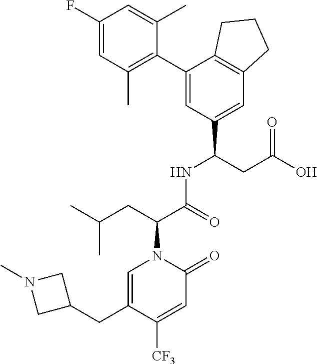 [SMILES: Cc1cc(F)cc(C)c1-c1cc([C@@H](CC(=O)O)NC(=O)[C@H](CC(C)C)n2cc(CC3CN(C)C3)c(C(F)(F)F)cc2=O)cc2c1CCC2] |
| | 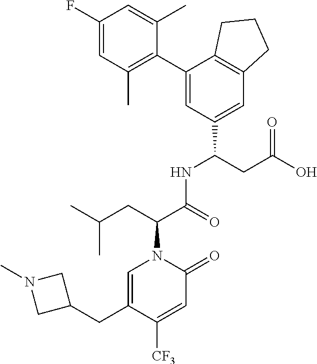 [SMILES: Cc1cc(F)cc(C)c1-c1cc([C@H](CC(=O)O)NC(=O)[C@H](CC(C)C)n2cc(CC3CN(C)C3)c(C(F)(F)F)cc2=O)cc2c1CCC2] |
| | 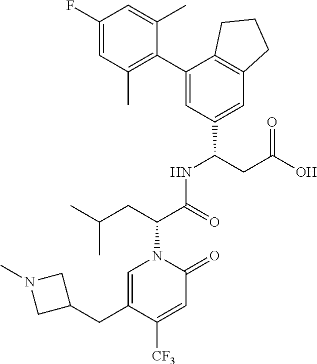 [SMILES: Cc1cc(F)cc(C)c1-c1cc([C@H](CC(=O)O)NC(=O)[C@@H](CC(C)C)n2cc(CC3CN(C)C3)c(C(F)(F)F)cc2=O)cc2c1CCC2] |
| | 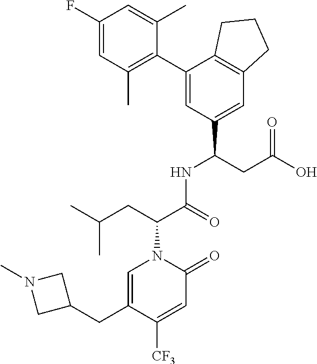 [SMILES: Cc1cc(F)cc(C)c1-c1cc([C@@H](CC(=O)O)NC(=O)[C@@H](CC(C)C)n2cc(CC3CN(C)C3)c(C(F)(F)F)cc2=O)cc2c1CCC2] |
| 34 | 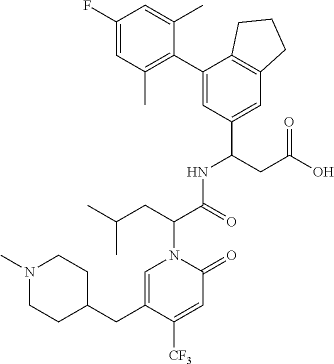 [SMILES: Cc1cc(F)cc(C)c1-c1cc(C(CC(=O)O)NC(=O)C(CC(C)C)n2cc(CC3CCN(C)CC3)c(C(F)(F)F)cc2=O)cc2c1CCC2] |
| | 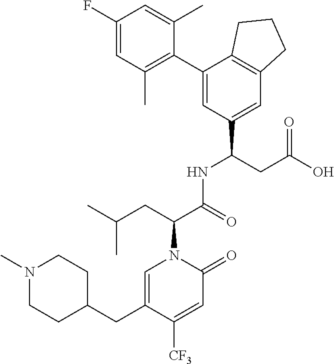 [SMILES: Cc1cc(F)cc(C)c1-c1cc([C@@H](CC(=O)O)NC(=O)[C@H](CC(C)C)n2cc(CC3CCN(C)CC3)c(C(F)(F)F)cc2=O)cc2c1CCC2] |
| | 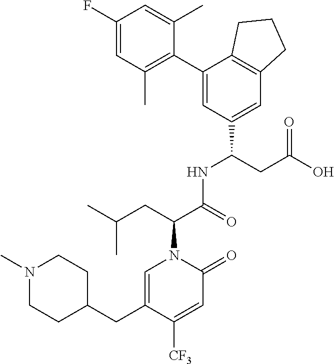 [SMILES: Cc1cc(F)cc(C)c1-c1cc([C@H](CC(=O)O)NC(=O)[C@H](CC(C)C)n2cc(CC3CCN(C)CC3)c(C(F)(F)F)cc2=O)cc2c1CCC2] |
| | 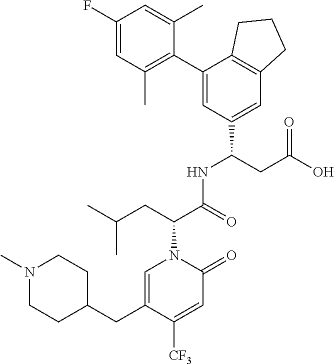 [SMILES: Cc1cc(F)cc(C)c1-c1cc([C@H](CC(=O)O)NC(=O)[C@@H](CC(C)C)n2cc(CC3CCN(C)CC3)c(C(F)(F)F)cc2=O)cc2c1CCC2] |
| | 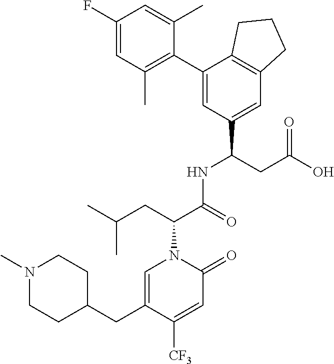 [SMILES: Cc1cc(F)cc(C)c1-c1cc([C@@H](CC(=O)O)NC(=O)[C@@H](CC(C)C)n2cc(CC3CCN(C)CC3)c(C(F)(F)F)cc2=O)cc2c1CCC2] |
| 35 | 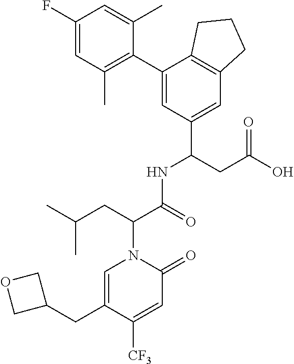 [SMILES: Cc1cc(F)cc(C)c1-c1cc(C(CC(=O)O)NC(=O)C(CC(C)C)n2cc(CC3COC3)c(C(F)(F)F)cc2=O)cc2c1CCC2] |
| | 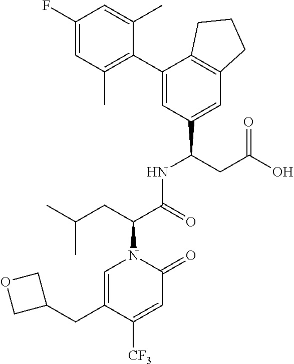 [SMILES: Cc1cc(F)cc(C)c1-c1cc([C@@H](CC(=O)O)NC(=O)[C@H](CC(C)C)n2cc(CC3COC3)c(C(F)(F)F)cc2=O)cc2c1CCC2] |
| | 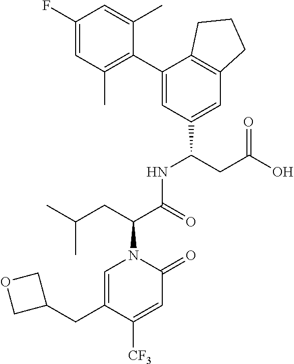 [SMILES: Cc1cc(F)cc(C)c1-c1cc([C@H](CC(=O)O)NC(=O)[C@H](CC(C)C)n2cc(CC3COC3)c(C(F)(F)F)cc2=O)cc2c1CCC2] |
| | 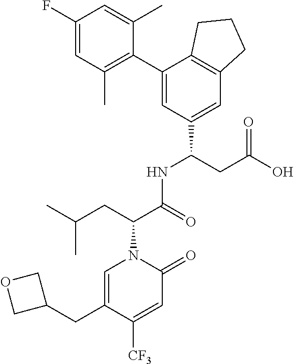 [SMILES: Cc1cc(F)cc(C)c1-c1cc([C@H](CC(=O)O)NC(=O)[C@@H](CC(C)C)n2cc(CC3COC3)c(C(F)(F)F)cc2=O)cc2c1CCC2] |
| | 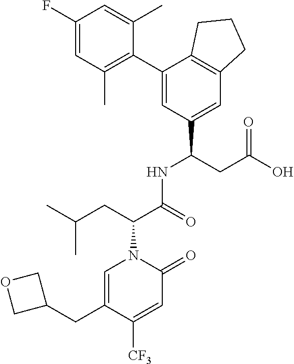 [SMILES: Cc1cc(F)cc(C)c1-c1cc([C@@H](CC(=O)O)NC(=O)[C@@H](CC(C)C)n2cc(CC3COC3)c(C(F)(F)F)cc2=O)cc2c1CCC2] |
| 36 | 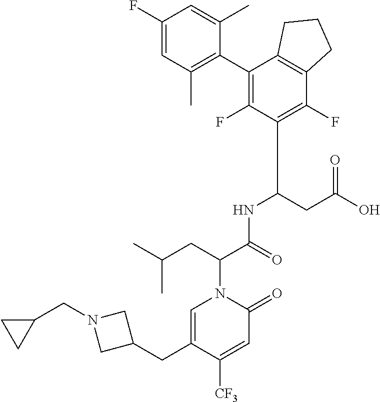 [SMILES: Cc1cc(F)cc(C)c1-c1c(F)c(C(CC(=O)O)NC(=O)C(CC(C)C)n2cc(CC3CN(CC4CC4)C3)c(C(F)(F)F)cc2=O)c(F)c2c1CCC2] |
| | 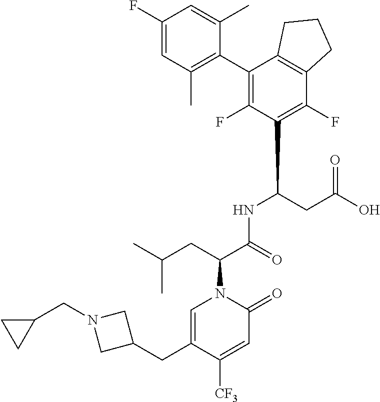 [SMILES: Cc1cc(F)cc(C)c1-c1c(F)c([C@@H](CC(=O)O)NC(=O)[C@H](CC(C)C)n2cc(CC3CN(CC4CC4)C3)c(C(F)(F)F)cc2=O)c(F)c2c1CCC2] |
| | 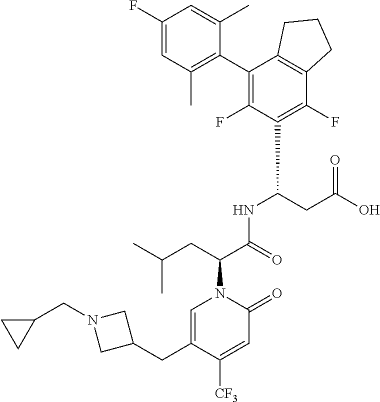 [SMILES: Cc1cc(F)cc(C)c1-c1c(F)c([C@H](CC(=O)O)NC(=O)[C@H](CC(C)C)n2cc(CC3CN(CC4CC4)C3)c(C(F)(F)F)cc2=O)c(F)c2c1CCC2] |
| | 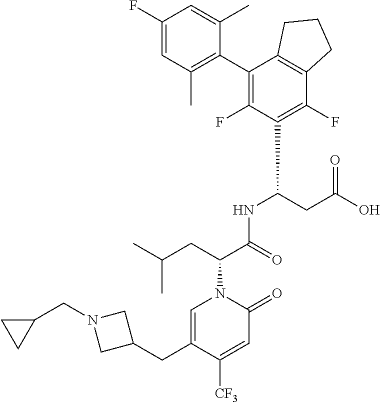 [SMILES: Cc1cc(F)cc(C)c1-c1c(F)c([C@H](CC(=O)O)NC(=O)[C@@H](CC(C)C)n2cc(CC3CN(CC4CC4)C3)c(C(F)(F)F)cc2=O)c(F)c2c1CCC2] |
| | 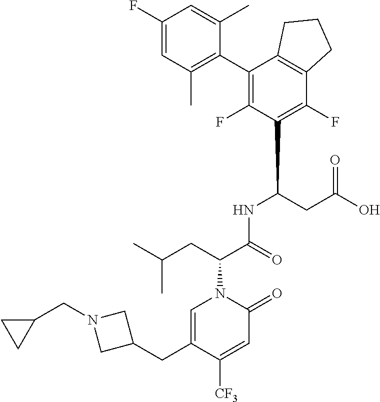 [SMILES: Cc1cc(F)cc(C)c1-c1c(F)c([C@@H](CC(=O)O)NC(=O)[C@@H](CC(C)C)n2cc(CC3CN(CC4CC4)C3)c(C(F)(F)F)cc2=O)c(F)c2c1CCC2] |
| 37 | 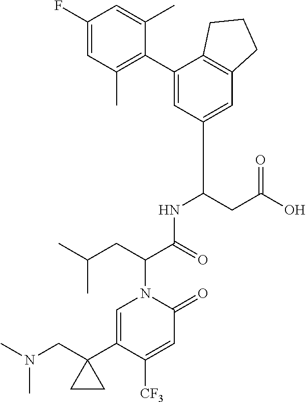 [SMILES: Cc1cc(F)cc(C)c1-c1cc(C(CC(=O)O)NC(=O)C(CC(C)C)n2cc(C3(CN(C)C)CC3)c(C(F)(F)F)cc2=O)cc2c1CCC2] |
| | 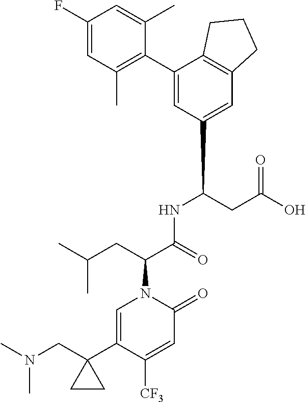 [SMILES: Cc1cc(F)cc(C)c1-c1cc([C@@H](CC(=O)O)NC(=O)[C@H](CC(C)C)n2cc(C3(CN(C)C)CC3)c(C(F)(F)F)cc2=O)cc2c1CCC2] |
| | 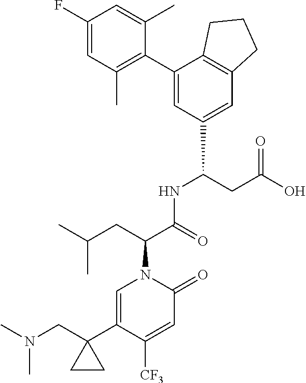 [SMILES: Cc1cc(F)cc(C)c1-c1cc([C@H](CC(=O)O)NC(=O)[C@H](CC(C)C)n2cc(C3(CN(C)C)CC3)c(C(F)(F)F)cc2=O)cc2c1CCC2] |
| | 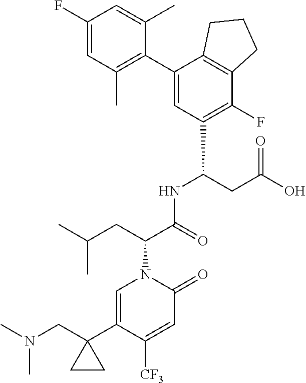 [SMILES: Cc1cc(F)cc(C)c1-c1cc([C@H](CC(=O)O)NC(=O)[C@@H](CC(C)C)n2cc(C3(CN(C)C)CC3)c(C(F)(F)F)cc2=O)c(F)c2c1CCC2] |
| | 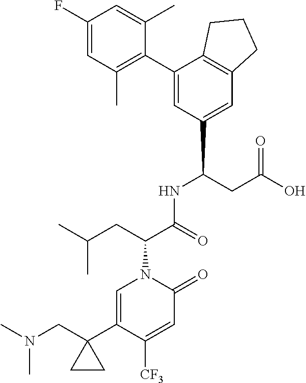 [SMILES: Cc1cc(F)cc(C)c1-c1cc([C@@H](CC(=O)O)NC(=O)[C@@H](CC(C)C)n2cc(C3(CN(C)C)CC3)c(C(F)(F)F)cc2=O)cc2c1CCC2] |
| 38 | 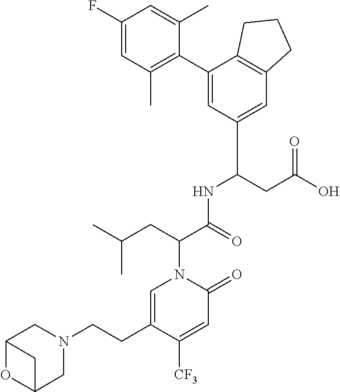 [SMILES: Cc1cc(F)cc(C)c1-c1cc(C(CC(=O)O)NC(=O)C(CC(C)C)n2cc(CCN3CC4CC(C3)O4)c(C(F)(F)F)cc2=O)cc2c1CCC2] |
| | 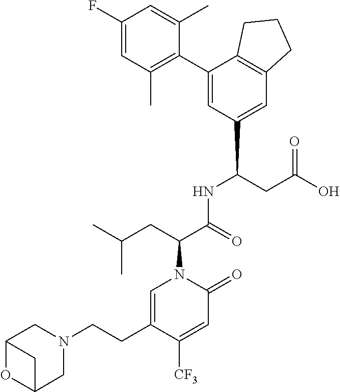 [SMILES: Cc1cc(F)cc(C)c1-c1cc([C@@H](CC(=O)O)NC(=O)[C@H](CC(C)C)n2cc(CCN3CC4CC(C3)O4)c(C(F)(F)F)cc2=O)cc2c1CCC2] |
| | 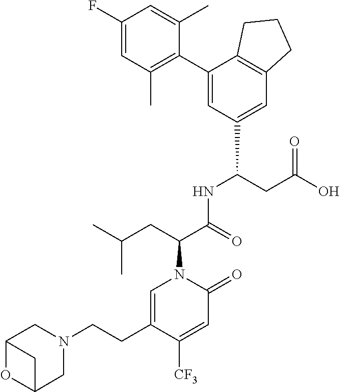 [SMILES: Cc1cc(F)cc(C)c1-c1cc([C@H](CC(=O)O)NC(=O)[C@H](CC(C)C)n2cc(CCN3CC4CC(C3)O4)c(C(F)(F)F)cc2=O)cc2c1CCC2] |
| | 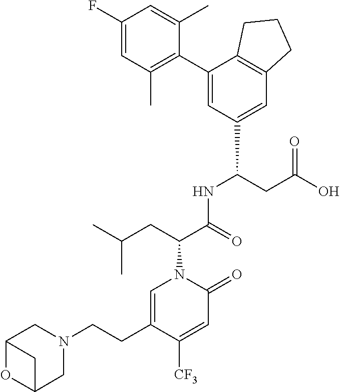 [SMILES: Cc1cc(F)cc(C)c1-c1cc([C@H](CC(=O)O)NC(=O)[C@@H](CC(C)C)n2cc(CCN3CC4CC(C3)O4)c(C(F)(F)F)cc2=O)cc2c1CCC2] |
| | 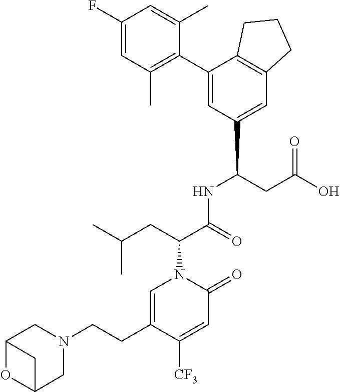 [SMILES: Cc1cc(F)cc(C)c1-c1cc([C@@H](CC(=O)O)NC(=O)[C@@H](CC(C)C)n2cc(CCN3CC4CC(C3)O4)c(C(F)(F)F)cc2=O)cc2c1CCC2] |
| 39 | 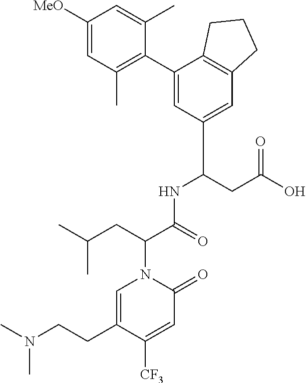 [SMILES: COc1cc(C)c(-c2cc(C(CC(=O)O)NC(=O)C(CC(C)C)n3cc(CCN(C)C)c(C(F)(F)F)cc3=O)cc3c2CCC3)c(C)c1] |
| | 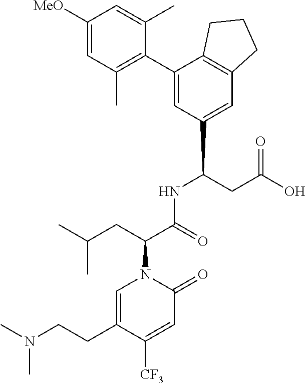 [SMILES: COc1cc(C)c(-c2cc([C@@H](CC(=O)O)NC(=O)[C@H](CC(C)C)n3cc(CCN(C)C)c(C(F)(F)F)cc3=O)cc3c2CCC3)c(C)c1] |
| | 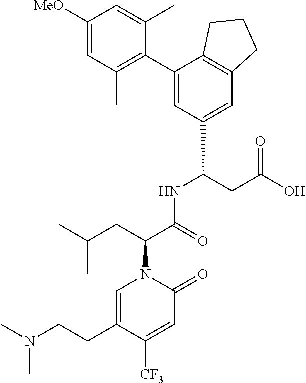 [SMILES: COc1cc(C)c(-c2cc([C@H](CC(=O)O)NC(=O)[C@H](CC(C)C)n3cc(CCN(C)C)c(C(F)(F)F)cc3=O)cc3c2CCC3)c(C)c1] |
| | 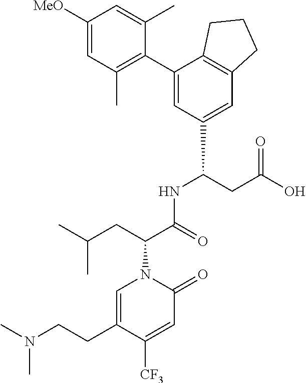 [SMILES: COc1cc(C)c(-c2cc([C@H](CC(=O)O)NC(=O)[C@@H](CC(C)C)n3cc(CCN(C)C)c(C(F)(F)F)cc3=O)cc3c2CCC3)c(C)c1] |
| | 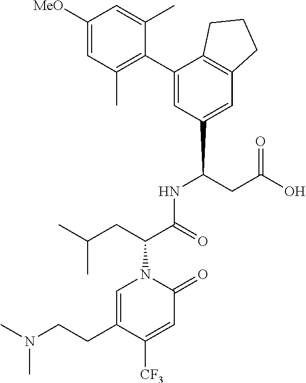 [SMILES: COc1cc(C)c(-c2cc([C@@H](CC(=O)O)NC(=O)[C@@H](CC(C)C)n3cc(CCN(C)C)c(C(F)(F)F)cc3=O)cc3c2CCC3)c(C)c1] |
| 40 | 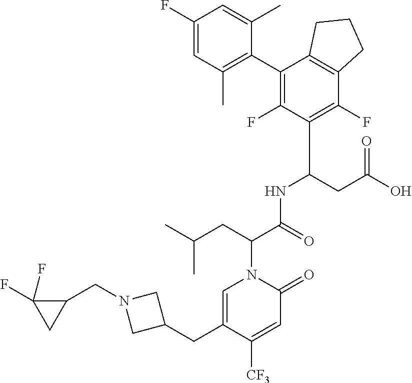 [SMILES: Cc1cc(F)cc(C)c1-c1c(F)c(C(CC(=O)O)NC(=O)C(CC(C)C)n2cc(CC3CN(CC4CC4(F)F)C3)c(C(F)(F)F)cc2=O)c(F)c2c1CCC2] |
| | 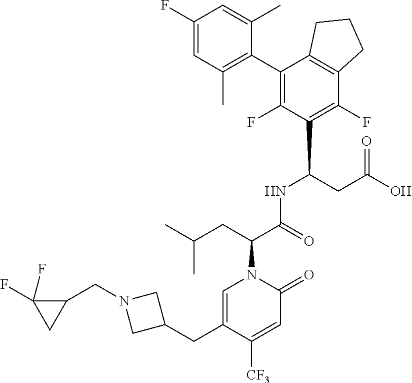 [SMILES: Cc1cc(F)cc(C)c1-c1c(F)c([C@@H](CC(=O)O)NC(=O)[C@H](CC(C)C)n2cc(CC3CN(CC4CC4(F)F)C3)c(C(F)(F)F)cc2=O)c(F)c2c1CCC2] |
| | 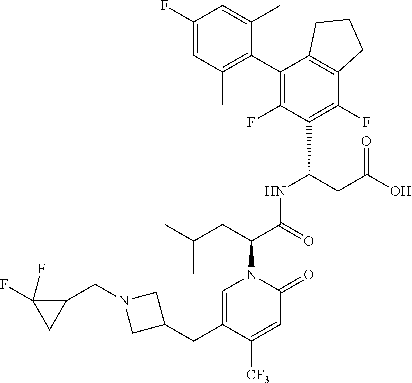 [SMILES: Cc1cc(F)cc(C)c1-c1c(F)c([C@H](CC(=O)O)NC(=O)[C@H](CC(C)C)n2cc(CC3CN(CC4CC4(F)F)C3)c(C(F)(F)F)cc2=O)c(F)c2c1CCC2] |
| | 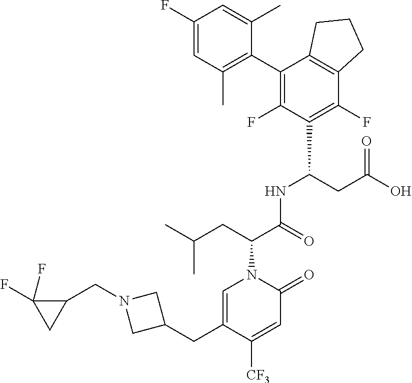 [SMILES: Cc1cc(F)cc(C)c1-c1c(F)c([C@H](CC(=O)O)NC(=O)[C@@H](CC(C)C)n2cc(CC3CN(CC4CC4(F)F)C3)c(C(F)(F)F)cc2=O)c(F)c2c1CCC2] |
| | 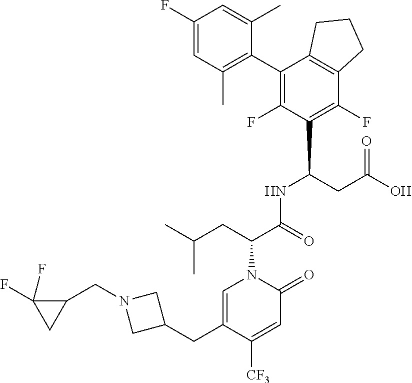 [SMILES: Cc1cc(F)cc(C)c1-c1c(F)c([C@@H](CC(=O)O)NC(=O)[C@@H](CC(C)C)n2cc(CC3CN(CC4CC4(F)F)C3)c(C(F)(F)F)cc2=O)c(F)c2c1CCC2] |
| 41 | 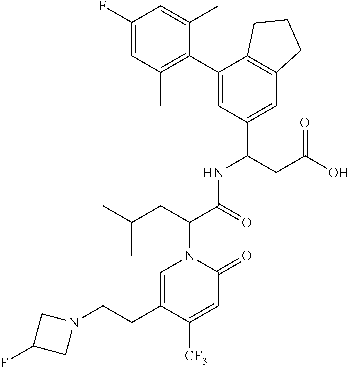 [SMILES: Cc1cc(F)cc(C)c1-c1cc(C(CC(=O)O)NC(=O)C(CC(C)C)n2cc(CCN3CC(F)C3)c(C(F)(F)F)cc2=O)cc2c1CCC2] |
| | 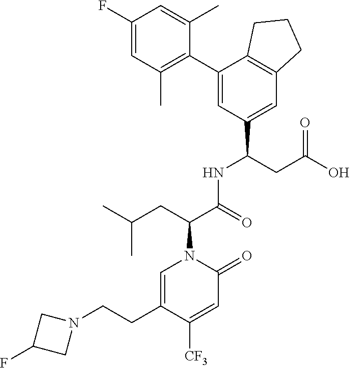 [SMILES: Cc1cc(F)cc(C)c1-c1cc([C@@H](CC(=O)O)NC(=O)[C@H](CC(C)C)n2cc(CCN3CC(F)C3)c(C(F)(F)F)cc2=O)cc2c1CCC2] |
| | 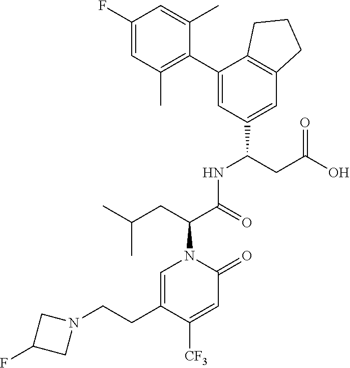 [SMILES: Cc1cc(F)cc(C)c1-c1cc([C@H](CC(=O)O)NC(=O)[C@H](CC(C)C)n2cc(CCN3CC(F)C3)c(C(F)(F)F)cc2=O)cc2c1CCC2] |
| | 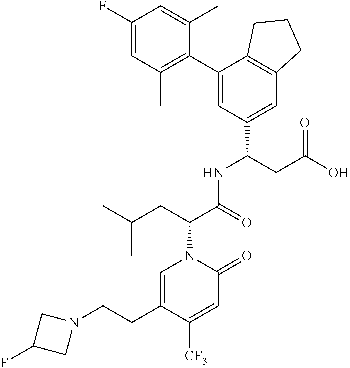 [SMILES: Cc1cc(F)cc(C)c1-c1cc([C@H](CC(=O)O)NC(=O)[C@@H](CC(C)C)n2cc(CCN3CC(F)C3)c(C(F)(F)F)cc2=O)cc2c1CCC2] |
| | 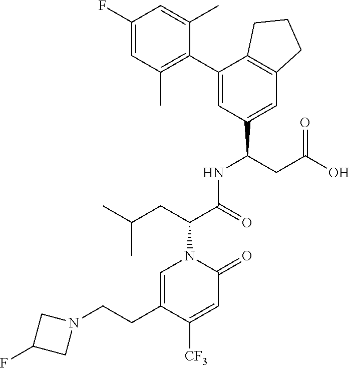 [SMILES: Cc1cc(F)cc(C)c1-c1cc([C@@H](CC(=O)O)NC(=O)[C@@H](CC(C)C)n2cc(CCN3CC(F)C3)c(C(F)(F)F)cc2=O)cc2c1CCC2] |
| 42 | 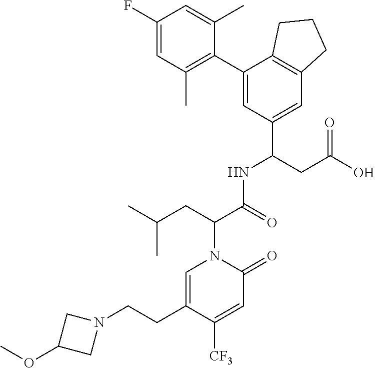 [SMILES: COC1CN(CCc2cn(C(CC(C)C)C(=O)NC(CC(=O)O)c3cc4c(c(-c5c(C)cc(F)cc5C)c3)CCC4)c(=O)cc2C(F)(F)F)C1] |
| | 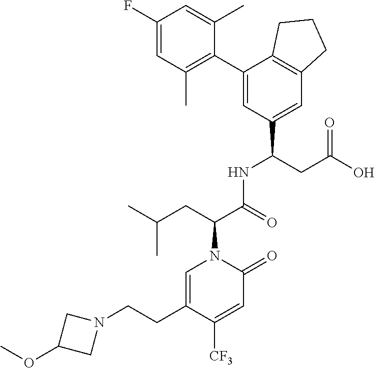 [SMILES: COC1CN(CCc2cn([C@@H](CC(C)C)C(=O)N[C@H](CC(=O)O)c3cc4c(c(-c5c(C)cc(F)cc5C)c3)CCC4)c(=O)cc2C(F)(F)F)C1] |
| | 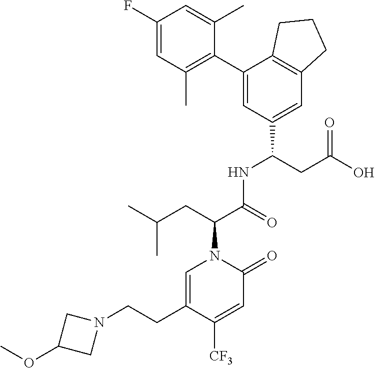 [SMILES: COC1CN(CCc2cn([C@@H](CC(C)C)C(=O)N[C@@H](CC(=O)O)c3cc4c(c(-c5c(C)cc(F)cc5C)c3)CCC4)c(=O)cc2C(F)(F)F)C1] |
| | 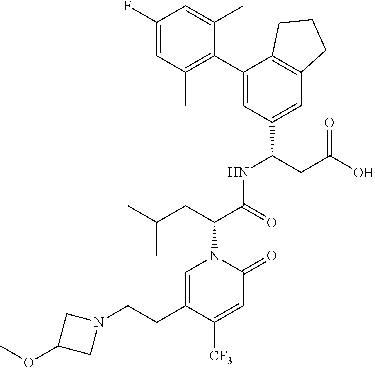 [SMILES: COC1CN(CCc2cn([C@H](CC(C)C)C(=O)N[C@@H](CC(=O)O)c3cc4c(c(-c5c(C)cc(F)cc5C)c3)CCC4)c(=O)cc2C(F)(F)F)C1] |
| | 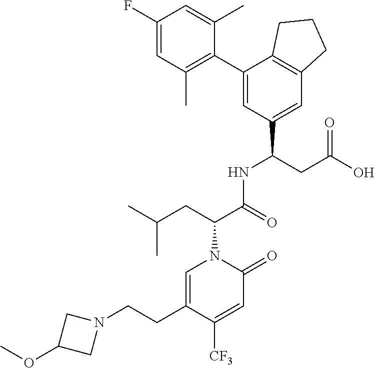 [SMILES: COC1CN(CCc2cn([C@H](CC(C)C)C(=O)N[C@H](CC(=O)O)c3cc4c(c(-c5c(C)cc(F)cc5C)c3)CCC4)c(=O)cc2C(F)(F)F)C1] |
| 43 | 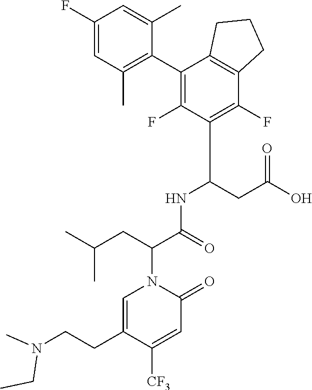 [SMILES: CCN(C)CCc1cn(C(CC(C)C)C(=O)NC(CC(=O)O)c2c(F)c3c(c(-c4c(C)cc(F)cc4C)c2F)CCC3)c(=O)cc1C(F)(F)F] |
| | 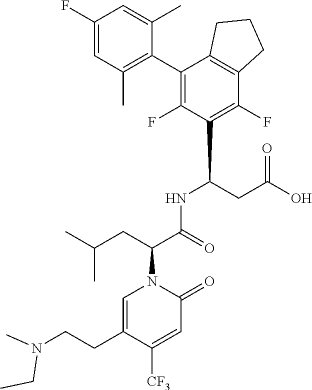 [SMILES: CCN(C)CCc1cn([C@@H](CC(C)C)C(=O)N[C@H](CC(=O)O)c2c(F)c3c(c(-c4c(C)cc(F)cc4C)c2F)CCC3)c(=O)cc1C(F)(F)F] |
| | 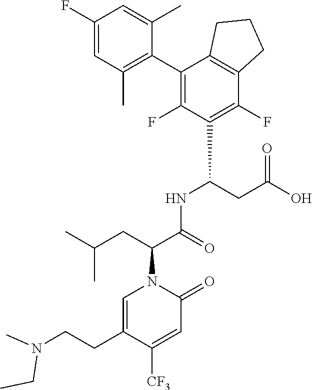 [SMILES: CCN(C)CCc1cn([C@@H](CC(C)C)C(=O)N[C@@H](CC(=O)O)c2c(F)c3c(c(-c4c(C)cc(F)cc4C)c2F)CCC3)c(=O)cc1C(F)(F)F] |
| | 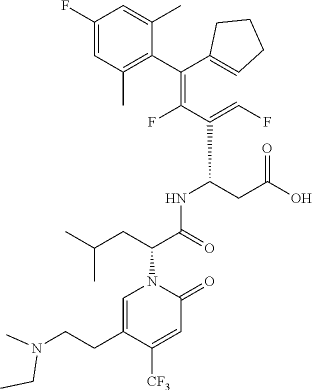 [SMILES: CCN(C)CCc1cn([C@H](CC(C)C)C(=O)N[C@@H](CC(=O)O)C(=C\F)/C(F)=C(\C2=CCCC2)c2c(C)cc(F)cc2C)c(=O)cc1C(F)(F)F] |
| | 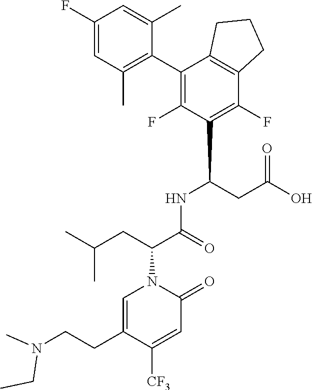 [SMILES: CCN(C)CCc1cn([C@H](CC(C)C)C(=O)N[C@H](CC(=O)O)c2c(F)c3c(c(-c4c(C)cc(F)cc4C)c2F)CCC3)c(=O)cc1C(F)(F)F] |
| 44 | 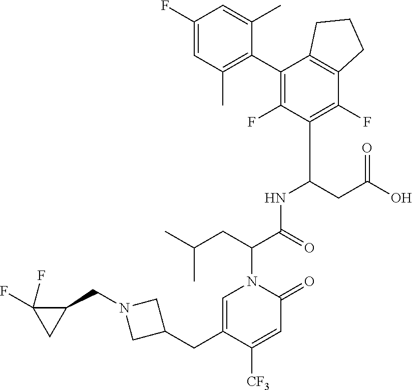 [SMILES: Cc1cc(F)cc(C)c1-c1c(F)c(C(CC(=O)O)NC(=O)C(CC(C)C)n2cc(CC3CN(C[C@H]4CC4(F)F)C3)c(C(F)(F)F)cc2=O)c(F)c2c1CCC2] |
| | 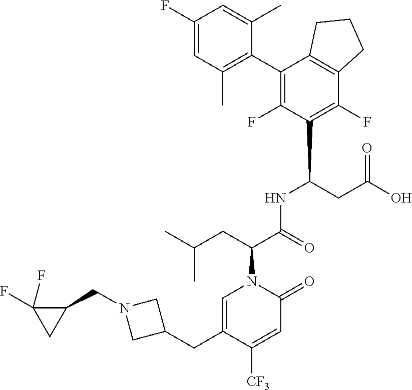 [SMILES: Cc1cc(F)cc(C)c1-c1c(F)c([C@@H](CC(=O)O)NC(=O)[C@H](CC(C)C)n2cc(CC3CN(C[C@H]4CC4(F)F)C3)c(C(F)(F)F)cc2=O)c(F)c2c1CCC2] |
| | 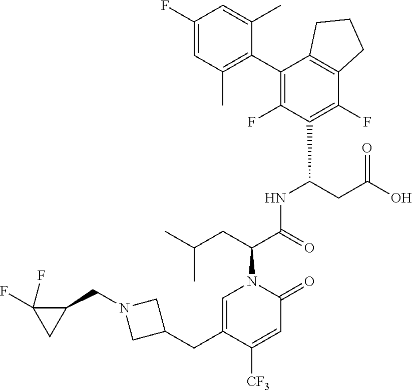 [SMILES: Cc1cc(F)cc(C)c1-c1c(F)c([C@H](CC(=O)O)NC(=O)[C@H](CC(C)C)n2cc(CC3CN(C[C@H]4CC4(F)F)C3)c(C(F)(F)F)cc2=O)c(F)c2c1CCC2] |
| | 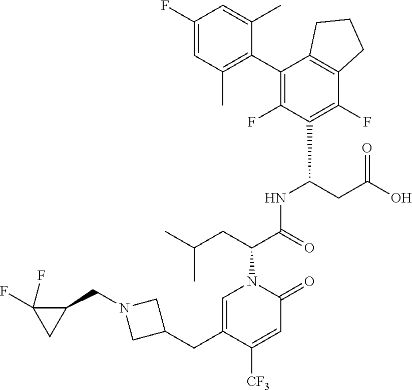 [SMILES: Cc1cc(F)cc(C)c1-c1c(F)c([C@H](CC(=O)O)NC(=O)[C@@H](CC(C)C)n2cc(CC3CN(C[C@H]4CC4(F)F)C3)c(C(F)(F)F)cc2=O)c(F)c2c1CCC2] |
| | 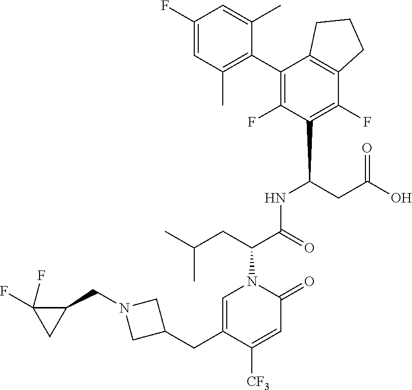 [SMILES: Cc1cc(F)cc(C)c1-c1c(F)c([C@@H](CC(=O)O)NC(=O)[C@@H](CC(C)C)n2cc(CC3CN(C[C@H]4CC4(F)F)C3)c(C(F)(F)F)cc2=O)c(F)c2c1CCC2] |
| 45 | 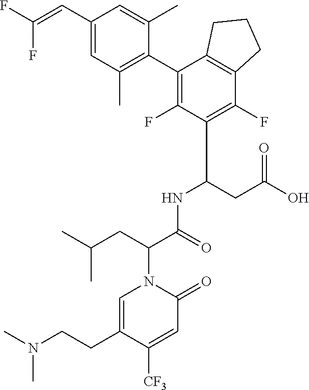 [SMILES: Cc1cc(C=C(F)F)cc(C)c1-c1c(F)c(C(CC(=O)O)NC(=O)C(CC(C)C)n2cc(CCN(C)C)c(C(F)(F)F)cc2=O)c(F)c2c1CCC2] |
| | 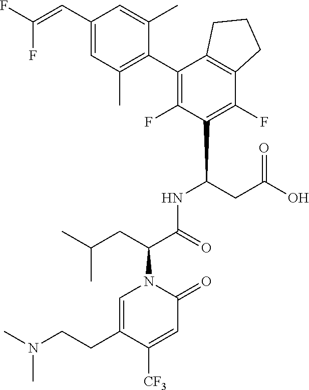 [SMILES: Cc1cc(C=C(F)F)cc(C)c1-c1c(F)c([C@@H](CC(=O)O)NC(=O)[C@H](CC(C)C)n2cc(CCN(C)C)c(C(F)(F)F)cc2=O)c(F)c2c1CCC2] |
| | 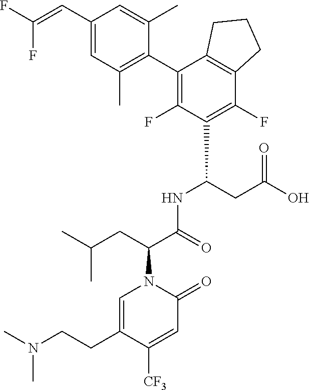 [SMILES: Cc1cc(C=C(F)F)cc(C)c1-c1c(F)c([C@H](CC(=O)O)NC(=O)[C@H](CC(C)C)n2cc(CCN(C)C)c(C(F)(F)F)cc2=O)c(F)c2c1CCC2] |
| | 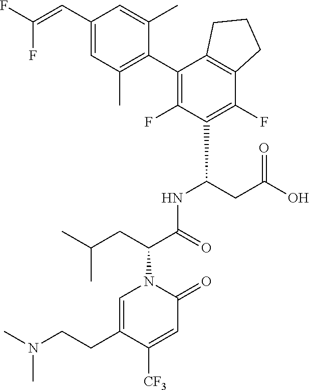 [SMILES: Cc1cc(C=C(F)F)cc(C)c1-c1c(F)c([C@H](CC(=O)O)NC(=O)[C@@H](CC(C)C)n2cc(CCN(C)C)c(C(F)(F)F)cc2=O)c(F)c2c1CCC2] |
| | 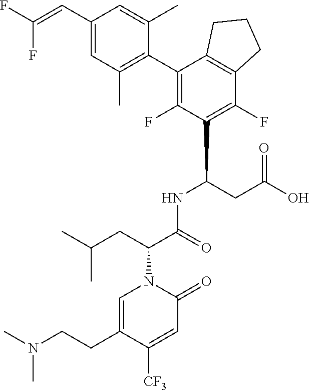 [SMILES: Cc1cc(C=C(F)F)cc(C)c1-c1c(F)c([C@@H](CC(=O)O)NC(=O)[C@@H](CC(C)C)n2cc(CCN(C)C)c(C(F)(F)F)cc2=O)c(F)c2c1CCC2] |
| 46 | 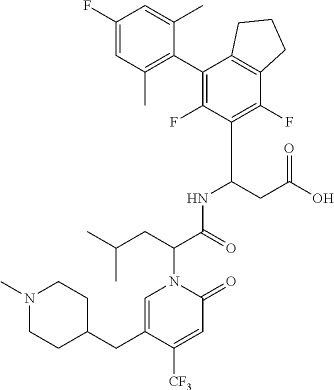 [SMILES: Cc1cc(F)cc(C)c1-c1c(F)c(C(CC(=O)O)NC(=O)C(CC(C)C)n2cc(CC3CCN(C)CC3)c(C(F)(F)F)cc2=O)c(F)c2c1CCC2] |
| | 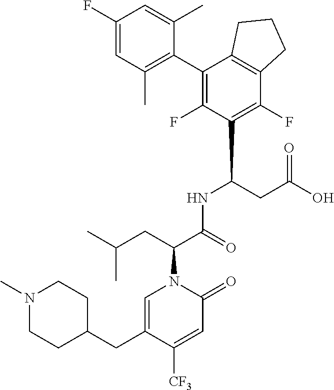 [SMILES: Cc1cc(F)cc(C)c1-c1c(F)c([C@@H](CC(=O)O)NC(=O)[C@H](CC(C)C)n2cc(CC3CCN(C)CC3)c(C(F)(F)F)cc2=O)c(F)c2c1CCC2] |
| | 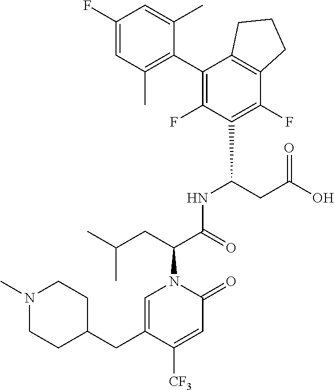 [SMILES: Cc1cc(F)cc(C)c1-c1c(F)c([C@H](CC(=O)O)NC(=O)[C@H](CC(C)C)n2cc(CC3CCN(C)CC3)c(C(F)(F)F)cc2=O)c(F)c2c1CCC2] |
| | 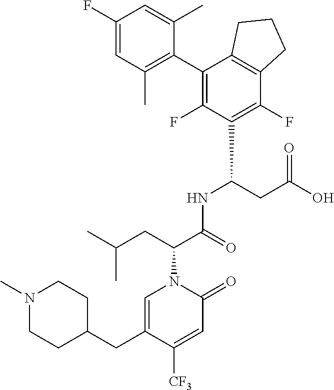 [SMILES: Cc1cc(F)cc(C)c1-c1c(F)c([C@H](CC(=O)O)NC(=O)[C@@H](CC(C)C)n2cc(CC3CCN(C)CC3)c(C(F)(F)F)cc2=O)c(F)c2c1CCC2] |
| | 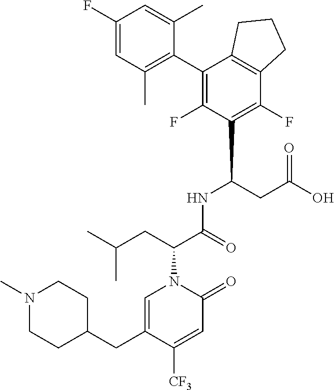 [SMILES: Cc1cc(F)cc(C)c1-c1c(F)c([C@@H](CC(=O)O)NC(=O)[C@@H](CC(C)C)n2cc(CC3CCN(C)CC3)c(C(F)(F)F)cc2=O)c(F)c2c1CCC2] |
| 47 | 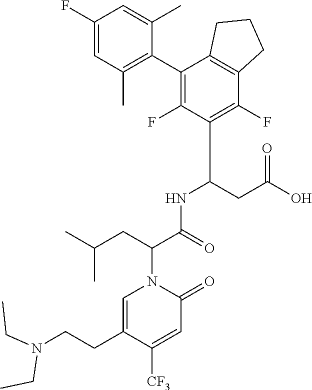 [SMILES: CCN(CC)CCc1cn(C(CC(C)C)C(=O)NC(CC(=O)O)c2c(F)c3c(c(-c4c(C)cc(F)cc4C)c2F)CCC3)c(=O)cc1C(F)(F)F] |
| | 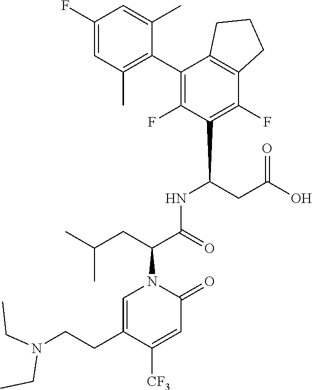 [SMILES: CCN(CC)CCc1cn([C@@H](CC(C)C)C(=O)N[C@H](CC(=O)O)c2c(F)c3c(c(-c4c(C)cc(F)cc4C)c2F)CCC3)c(=O)cc1C(F)(F)F] |
| | 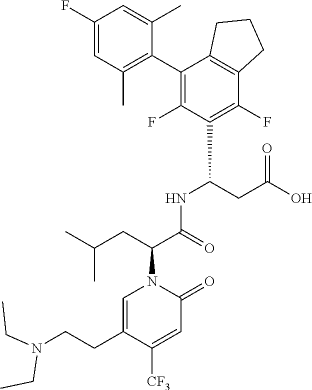 [SMILES: CCN(CC)CCc1cn([C@@H](CC(C)C)C(=O)N[C@@H](CC(=O)O)c2c(F)c3c(c(-c4c(C)cc(F)cc4C)c2F)CCC3)c(=O)cc1C(F)(F)F] |
| | 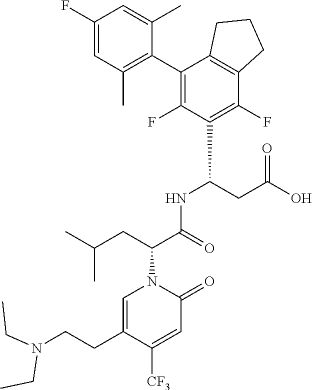 [SMILES: CCN(CC)CCc1cn([C@H](CC(C)C)C(=O)N[C@@H](CC(=O)O)c2c(F)c3c(c(-c4c(C)cc(F)cc4C)c2F)CCC3)c(=O)cc1C(F)(F)F] |
| | 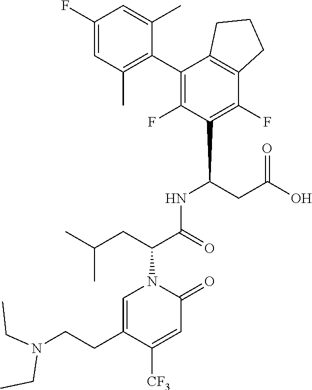 [SMILES: CCN(CC)CCc1cn([C@H](CC(C)C)C(=O)N[C@H](CC(=O)O)c2c(F)c3c(c(-c4c(C)cc(F)cc4C)c2F)CCC3)c(=O)cc1C(F)(F)F] |
| 48 | 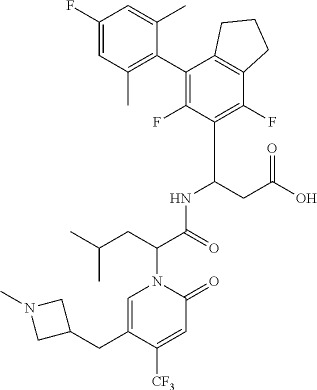 [SMILES: Cc1cc(F)cc(C)c1-c1c(F)c(C(CC(=O)O)NC(=O)C(CC(C)C)n2cc(CC3CN(C)C3)c(C(F)(F)F)cc2=O)c(F)c2c1CCC2] |
| | 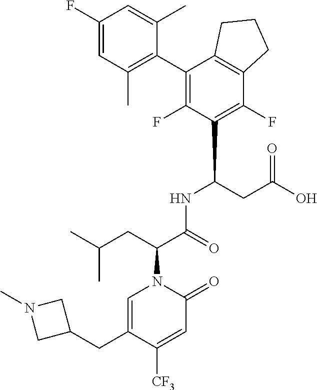 [SMILES: Cc1cc(F)cc(C)c1-c1c(F)c([C@@H](CC(=O)O)NC(=O)[C@H](CC(C)C)n2cc(CC3CN(C)C3)c(C(F)(F)F)cc2=O)c(F)c2c1CCC2] |
| | 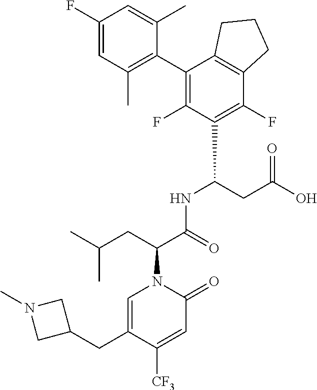 [SMILES: Cc1cc(F)cc(C)c1-c1c(F)c([C@H](CC(=O)O)NC(=O)[C@H](CC(C)C)n2cc(CC3CN(C)C3)c(C(F)(F)F)cc2=O)c(F)c2c1CCC2] |
| | 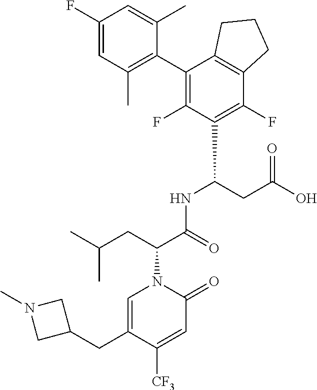 [SMILES: Cc1cc(F)cc(C)c1-c1c(F)c([C@H](CC(=O)O)NC(=O)[C@@H](CC(C)C)n2cc(CC3CN(C)C3)c(C(F)(F)F)cc2=O)c(F)c2c1CCC2] |
| | 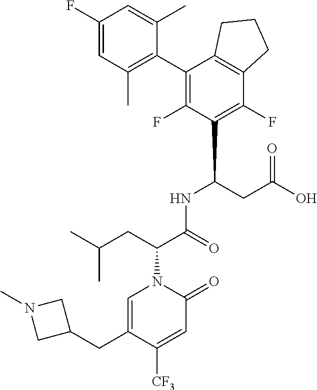 [SMILES: Cc1cc(F)cc(C)c1-c1c(F)c([C@@H](CC(=O)O)NC(=O)[C@@H](CC(C)C)n2cc(CC3CN(C)C3)c(C(F)(F)F)cc2=O)c(F)c2c1CCC2] |
| 49 | 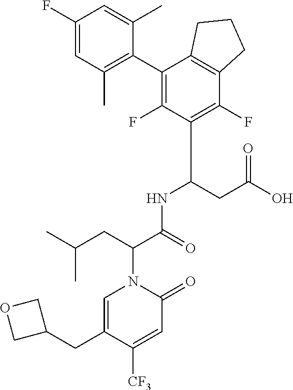 [SMILES: Cc1cc(F)cc(C)c1-c1c(F)c(C(CC(=O)O)NC(=O)C(CC(C)C)n2cc(CC3COC3)c(C(F)(F)F)cc2=O)c(F)c2c1CCC2] |
| | 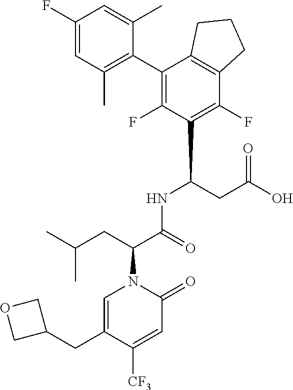 [SMILES: Cc1cc(F)cc(C)c1-c1c(F)c([C@@H](CC(=O)O)NC(=O)[C@H](CC(C)C)n2cc(CC3COC3)c(C(F)(F)F)cc2=O)c(F)c2c1CCC2] |
| | 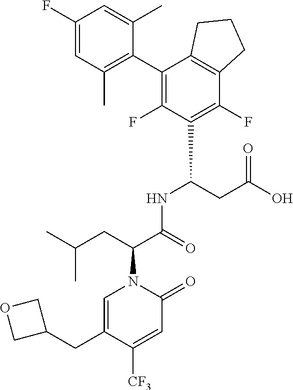 [SMILES: Cc1cc(F)cc(C)c1-c1c(F)c([C@H](CC(=O)O)NC(=O)[C@H](CC(C)C)n2cc(CC3COC3)c(C(F)(F)F)cc2=O)c(F)c2c1CCC2] |
| | 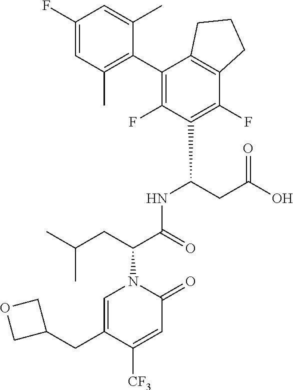 [SMILES: Cc1cc(F)cc(C)c1-c1c(F)c([C@H](CC(=O)O)NC(=O)[C@@H](CC(C)C)n2cc(CC3COC3)c(C(F)(F)F)cc2=O)c(F)c2c1CCC2] |
| | 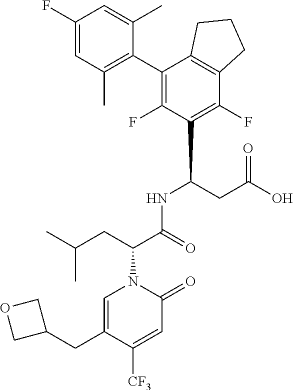 [SMILES: Cc1cc(F)cc(C)c1-c1c(F)c([C@@H](CC(=O)O)NC(=O)[C@@H](CC(C)C)n2cc(CC3COC3)c(C(F)(F)F)cc2=O)c(F)c2c1CCC2] |
| 50 | 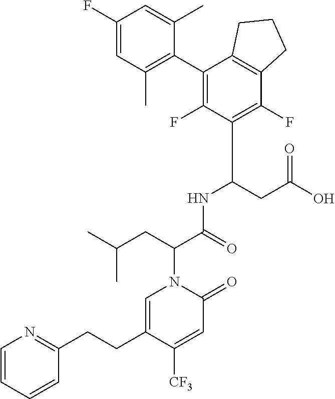 [SMILES: Cc1cc(F)cc(C)c1-c1c(F)c(C(CC(=O)O)NC(=O)C(CC(C)C)n2cc(CCc3ccccn3)c(C(F)(F)F)cc2=O)c(F)c2c1CCC2] |
| | 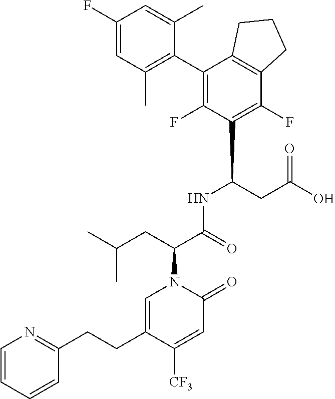 [SMILES: Cc1cc(F)cc(C)c1-c1c(F)c([C@@H](CC(=O)O)NC(=O)[C@H](CC(C)C)n2cc(CCc3ccccn3)c(C(F)(F)F)cc2=O)c(F)c2c1CCC2] |
| | 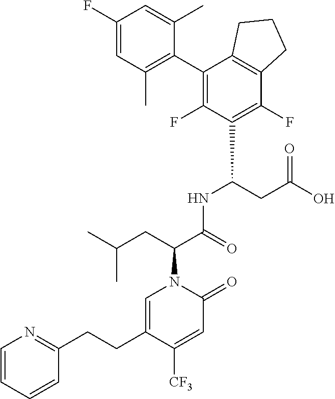 [SMILES: Cc1cc(F)cc(C)c1-c1c(F)c([C@H](CC(=O)O)NC(=O)[C@H](CC(C)C)n2cc(CCc3ccccn3)c(C(F)(F)F)cc2=O)c(F)c2c1CCC2] |
| | 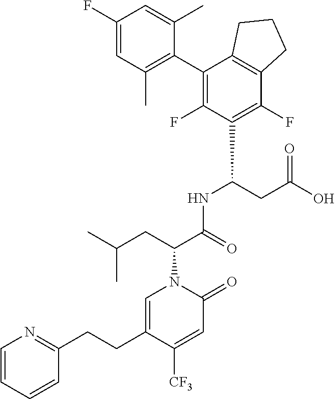 [SMILES: Cc1cc(F)cc(C)c1-c1c(F)c([C@H](CC(=O)O)NC(=O)[C@@H](CC(C)C)n2cc(CCc3ccccn3)c(C(F)(F)F)cc2=O)c(F)c2c1CCC2] |
| | 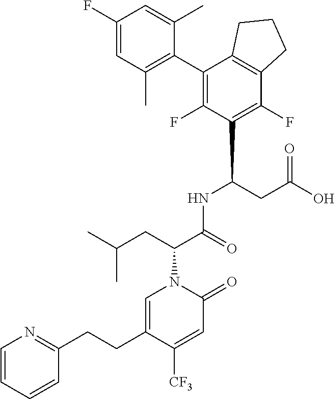 [SMILES: Cc1cc(F)cc(C)c1-c1c(F)c([C@@H](CC(=O)O)NC(=O)[C@@H](CC(C)C)n2cc(CCc3ccccn3)c(C(F)(F)F)cc2=O)c(F)c2c1CCC2] |
| 51 | 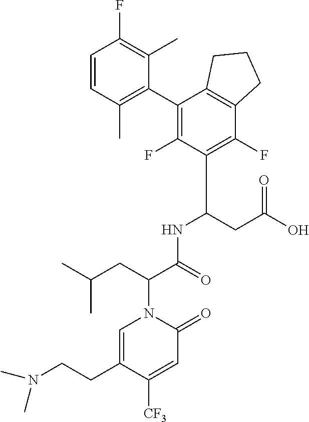 [SMILES: Cc1ccc(F)c(C)c1-c1c(F)c(C(CC(=O)O)NC(=O)C(CC(C)C)n2cc(CCN(C)C)c(C(F)(F)F)cc2=O)c(F)c2c1CCC2] |
| | 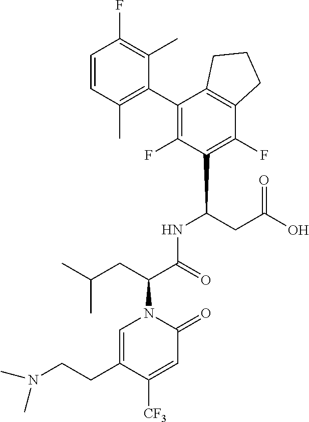 [SMILES: Cc1ccc(F)c(C)c1-c1c(F)c([C@@H](CC(=O)O)NC(=O)[C@H](CC(C)C)n2cc(CCN(C)C)c(C(F)(F)F)cc2=O)c(F)c2c1CCC2] |
| | 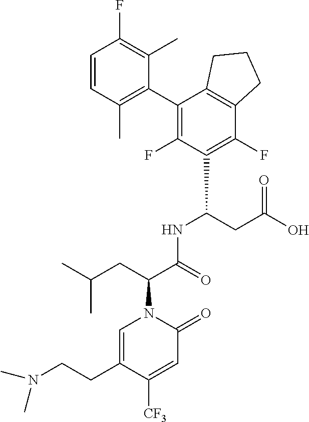 [SMILES: Cc1ccc(F)c(C)c1-c1c(F)c([C@H](CC(=O)O)NC(=O)[C@H](CC(C)C)n2cc(CCN(C)C)c(C(F)(F)F)cc2=O)c(F)c2c1CCC2] |
| | 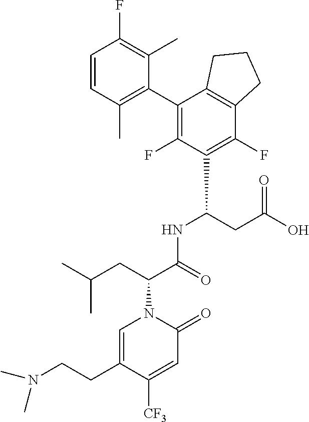 [SMILES: Cc1ccc(F)c(C)c1-c1c(F)c([C@H](CC(=O)O)NC(=O)[C@@H](CC(C)C)n2cc(CCN(C)C)c(C(F)(F)F)cc2=O)c(F)c2c1CCC2] |
| | 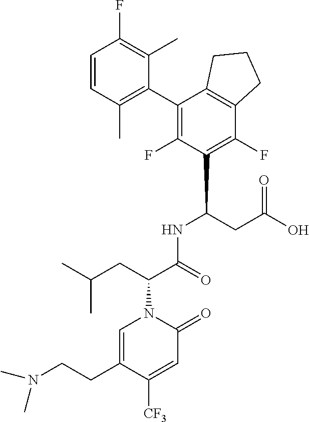 [SMILES: Cc1ccc(F)c(C)c1-c1c(F)c([C@@H](CC(=O)O)NC(=O)[C@@H](CC(C)C)n2cc(CCN(C)C)c(C(F)(F)F)cc2=O)c(F)c2c1CCC2] |
| 52 | 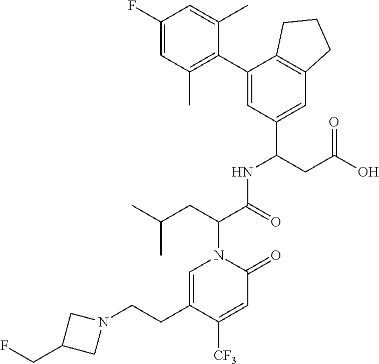 [SMILES: Cc1cc(F)cc(C)c1-c1cc(C(CC(=O)O)NC(=O)C(CC(C)C)n2cc(CCN3CC(CF)C3)c(C(F)(F)F)cc2=O)cc2c1CCC2] |
| | 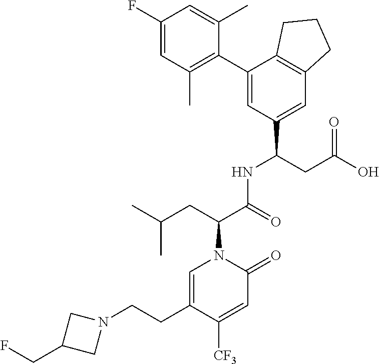 [SMILES: Cc1cc(F)cc(C)c1-c1cc([C@@H](CC(=O)O)NC(=O)[C@H](CC(C)C)n2cc(CCN3CC(CF)C3)c(C(F)(F)F)cc2=O)cc2c1CCC2] |
| | 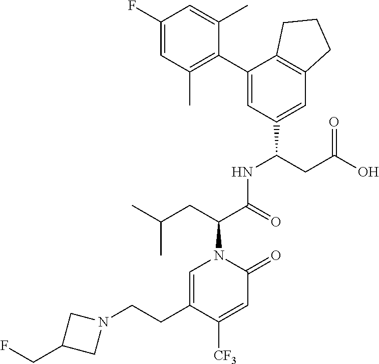 [SMILES: Cc1cc(F)cc(C)c1-c1cc([C@H](CC(=O)O)NC(=O)[C@H](CC(C)C)n2cc(CCN3CC(CF)C3)c(C(F)(F)F)cc2=O)cc2c1CCC2] |
| | 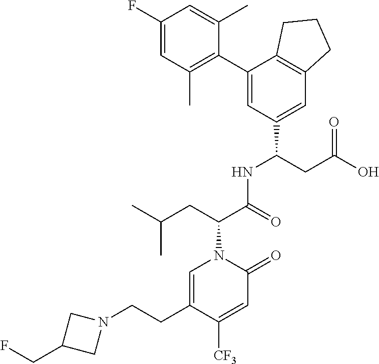 [SMILES: Cc1cc(F)cc(C)c1-c1cc([C@H](CC(=O)O)NC(=O)[C@@H](CC(C)C)n2cc(CCN3CC(CF)C3)c(C(F)(F)F)cc2=O)cc2c1CCC2] |
| | 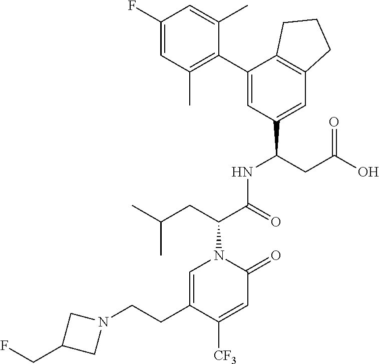 [SMILES: Cc1cc(F)cc(C)c1-c1cc([C@@H](CC(=O)O)NC(=O)[C@@H](CC(C)C)n2cc(CCN3CC(CF)C3)c(C(F)(F)F)cc2=O)cc2c1CCC2] |
| 53 | 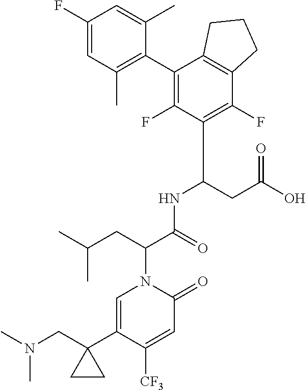 [SMILES: Cc1cc(F)cc(C)c1-c1c(F)c(C(CC(=O)O)NC(=O)C(CC(C)C)n2cc(C3(CN(C)C)CC3)c(C(F)(F)F)cc2=O)c(F)c2c1CCC2] |
| | 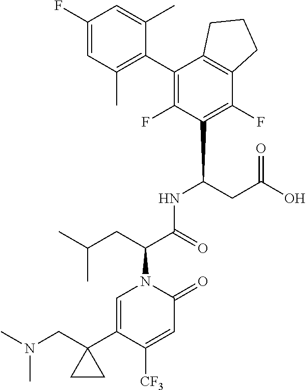 [SMILES: Cc1cc(F)cc(C)c1-c1c(F)c([C@@H](CC(=O)O)NC(=O)[C@H](CC(C)C)n2cc(C3(CN(C)C)CC3)c(C(F)(F)F)cc2=O)c(F)c2c1CCC2] |
| | 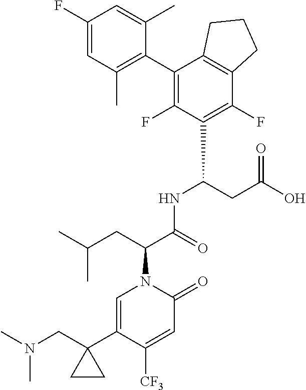 [SMILES: Cc1cc(F)cc(C)c1-c1c(F)c([C@H](CC(=O)O)NC(=O)[C@H](CC(C)C)n2cc(C3(CN(C)C)CC3)c(C(F)(F)F)cc2=O)c(F)c2c1CCC2] |
| | 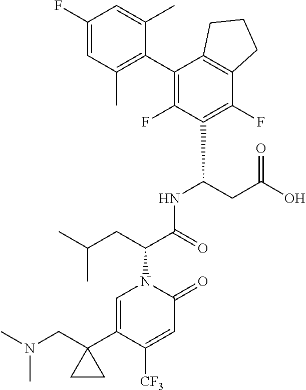 [SMILES: Cc1cc(F)cc(C)c1-c1c(F)c([C@H](CC(=O)O)NC(=O)[C@@H](CC(C)C)n2cc(C3(CN(C)C)CC3)c(C(F)(F)F)cc2=O)c(F)c2c1CCC2] |
| | 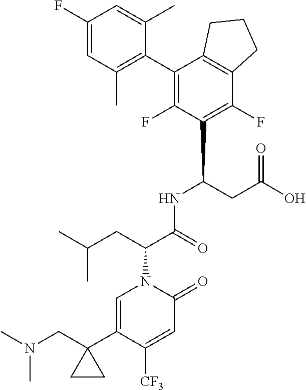 [SMILES: Cc1cc(F)cc(C)c1-c1c(F)c([C@@H](CC(=O)O)NC(=O)[C@@H](CC(C)C)n2cc(C3(CN(C)C)CC3)c(C(F)(F)F)cc2=O)c(F)c2c1CCC2] |
| 54 | 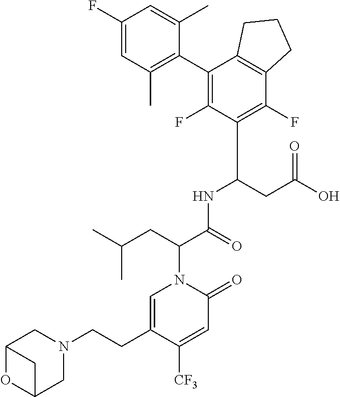 [SMILES: Cc1cc(F)cc(C)c1-c1c(F)c(C(CC(=O)O)NC(=O)C(CC(C)C)n2cc(CCN3CC4CC(C3)O4)c(C(F)(F)F)cc2=O)c(F)c2c1CCC2] |
| | 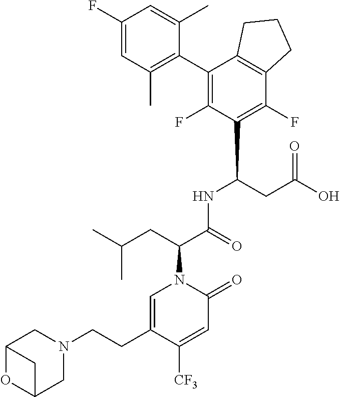 [SMILES: Cc1cc(F)cc(C)c1-c1c(F)c([C@@H](CC(=O)O)NC(=O)[C@H](CC(C)C)n2cc(CCN3CC4CC(C3)O4)c(C(F)(F)F)cc2=O)c(F)c2c1CCC2] |
| | 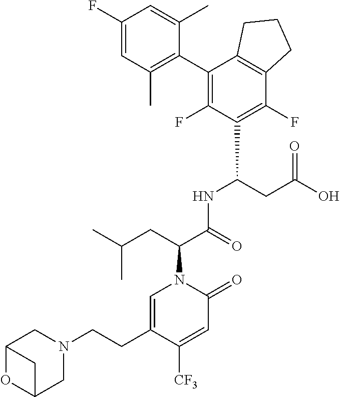 [SMILES: Cc1cc(F)cc(C)c1-c1c(F)c([C@H](CC(=O)O)NC(=O)[C@H](CC(C)C)n2cc(CCN3CC4CC(C3)O4)c(C(F)(F)F)cc2=O)c(F)c2c1CCC2] |
| | 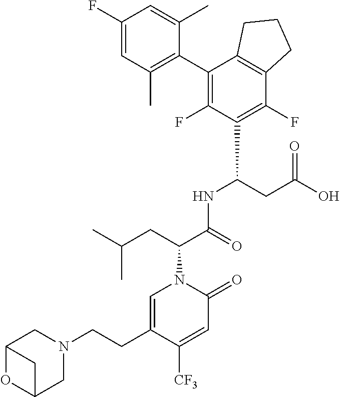 [SMILES: Cc1cc(F)cc(C)c1-c1c(F)c([C@H](CC(=O)O)NC(=O)[C@@H](CC(C)C)n2cc(CCN3CC4CC(C3)O4)c(C(F)(F)F)cc2=O)c(F)c2c1CCC2] |
| | 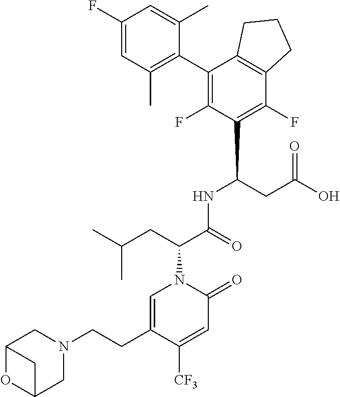 [SMILES: Cc1cc(F)cc(C)c1-c1c(F)c([C@@H](CC(=O)O)NC(=O)[C@@H](CC(C)C)n2cc(CCN3CC4CC(C3)O4)c(C(F)(F)F)cc2=O)c(F)c2c1CCC2] |
| 55 | 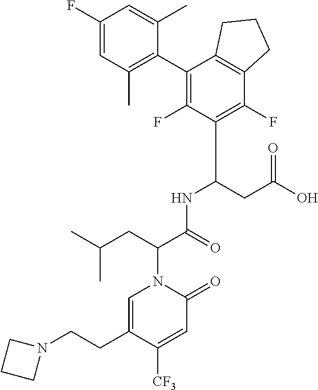 [SMILES: Cc1cc(F)cc(C)c1-c1c(F)c(C(CC(=O)O)NC(=O)C(CC(C)C)n2cc(CCN3CCC3)c(C(F)(F)F)cc2=O)c(F)c2c1CCC2] |
| | 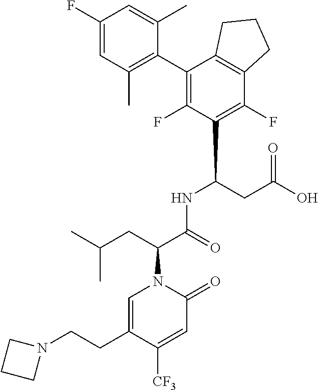 [SMILES: Cc1cc(F)cc(C)c1-c1c(F)c([C@@H](CC(=O)O)NC(=O)[C@H](CC(C)C)n2cc(CCN3CCC3)c(C(F)(F)F)cc2=O)c(F)c2c1CCC2] |
| | 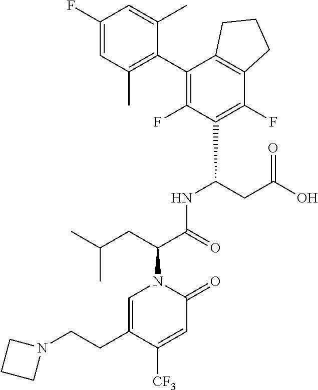 [SMILES: Cc1cc(F)cc(C)c1-c1c(F)c([C@H](CC(=O)O)NC(=O)[C@H](CC(C)C)n2cc(CCN3CCC3)c(C(F)(F)F)cc2=O)c(F)c2c1CCC2] |
| | 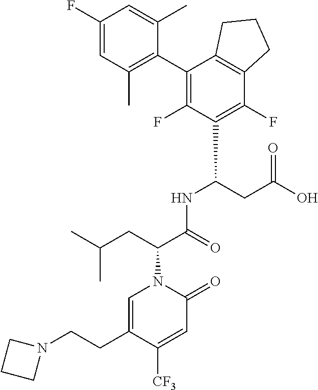 [SMILES: Cc1cc(F)cc(C)c1-c1c(F)c([C@H](CC(=O)O)NC(=O)[C@@H](CC(C)C)n2cc(CCN3CCC3)c(C(F)(F)F)cc2=O)c(F)c2c1CCC2] |
| | 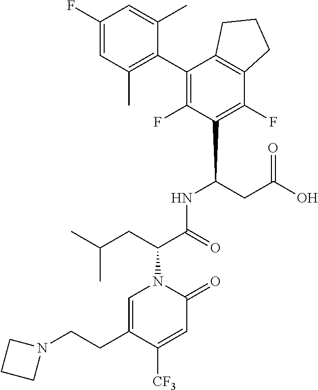 [SMILES: Cc1cc(F)cc(C)c1-c1c(F)c([C@@H](CC(=O)O)NC(=O)[C@@H](CC(C)C)n2cc(CCN3CCC3)c(C(F)(F)F)cc2=O)c(F)c2c1CCC2] |
| 56 | 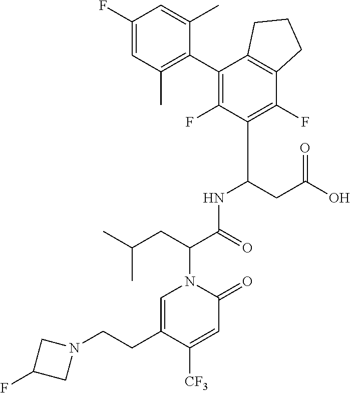 [SMILES: Cc1cc(F)cc(C)c1-c1c(F)c(C(CC(=O)O)NC(=O)C(CC(C)C)n2cc(CCN3CC(F)C3)c(C(F)(F)F)cc2=O)c(F)c2c1CCC2] |
| | 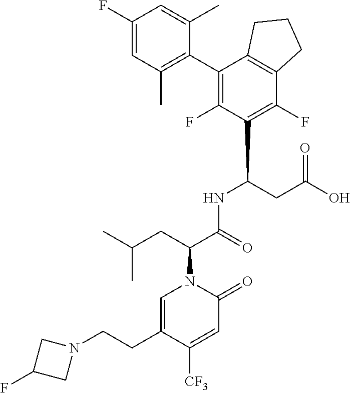 [SMILES: Cc1cc(F)cc(C)c1-c1c(F)c([C@@H](CC(=O)O)NC(=O)[C@H](CC(C)C)n2cc(CCN3CC(F)C3)c(C(F)(F)F)cc2=O)c(F)c2c1CCC2] |
| | 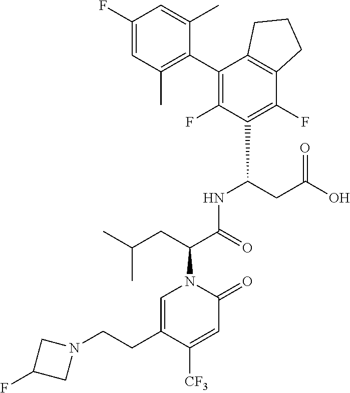 [SMILES: Cc1cc(F)cc(C)c1-c1c(F)c([C@H](CC(=O)O)NC(=O)[C@H](CC(C)C)n2cc(CCN3CC(F)C3)c(C(F)(F)F)cc2=O)c(F)c2c1CCC2] |
| | 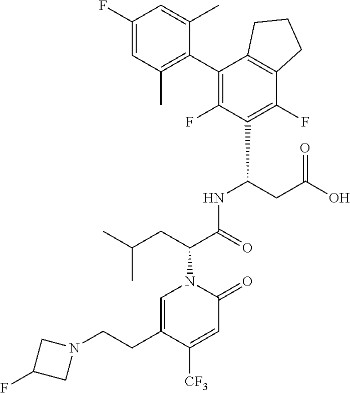 [SMILES: Cc1cc(F)cc(C)c1-c1c(F)c([C@H](CC(=O)O)NC(=O)[C@@H](CC(C)C)n2cc(CCN3CC(F)C3)c(C(F)(F)F)cc2=O)c(F)c2c1CCC2] |
| | 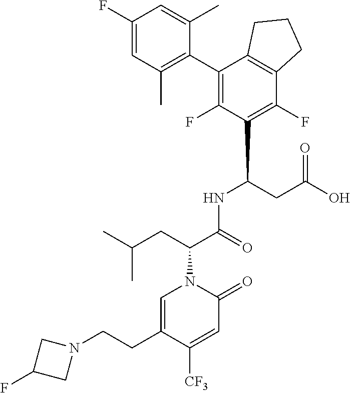 [SMILES: Cc1cc(F)cc(C)c1-c1c(F)c([C@@H](CC(=O)O)NC(=O)[C@@H](CC(C)C)n2cc(CCN3CC(F)C3)c(C(F)(F)F)cc2=O)c(F)c2c1CCC2] |
| 57 | 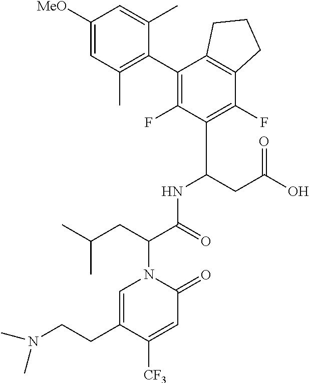 [SMILES: COc1cc(C)c(-c2c(F)c(C(CC(=O)O)NC(=O)C(CC(C)C)n3cc(CCN(C)C)c(C(F)(F)F)cc3=O)c(F)c3c2CCC3)c(C)c1] |
| | 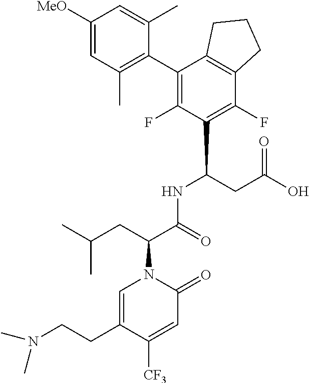 [SMILES: COc1cc(C)c(-c2c(F)c([C@@H](CC(=O)O)NC(=O)[C@H](CC(C)C)n3cc(CCN(C)C)c(C(F)(F)F)cc3=O)c(F)c3c2CCC3)c(C)c1] |
| | 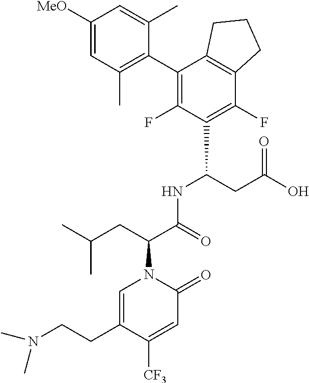 [SMILES: COc1cc(C)c(-c2c(F)c([C@H](CC(=O)O)NC(=O)[C@H](CC(C)C)n3cc(CCN(C)C)c(C(F)(F)F)cc3=O)c(F)c3c2CCC3)c(C)c1] |
| | 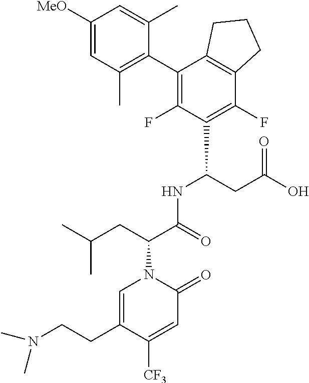 [SMILES: COc1cc(C)c(-c2c(F)c([C@H](CC(=O)O)NC(=O)[C@@H](CC(C)C)n3cc(CCN(C)C)c(C(F)(F)F)cc3=O)c(F)c3c2CCC3)c(C)c1] |
| | 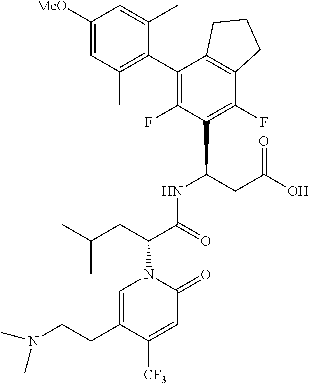 [SMILES: COc1cc(C)c(-c2c(F)c([C@@H](CC(=O)O)NC(=O)[C@@H](CC(C)C)n3cc(CCN(C)C)c(C(F)(F)F)cc3=O)c(F)c3c2CCC3)c(C)c1] |
| 58 | 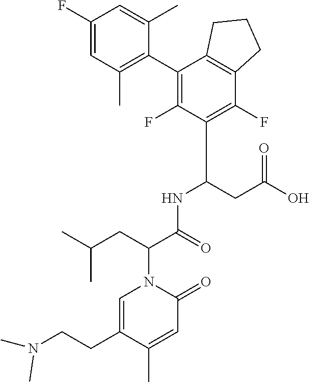 [SMILES: Cc1cc(=O)n(C(CC(C)C)C(=O)NC(CC(=O)O)c2c(F)c3c(c(-c4c(C)cc(F)cc4C)c2F)CCC3)cc1CCN(C)C] |
| | 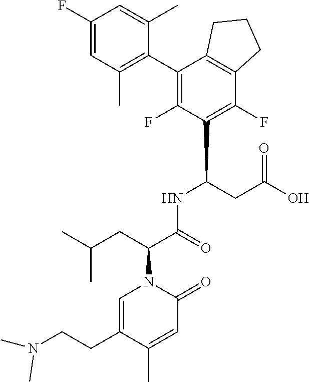 [SMILES: Cc1cc(=O)n([C@@H](CC(C)C)C(=O)N[C@H](CC(=O)O)c2c(F)c3c(c(-c4c(C)cc(F)cc4C)c2F)CCC3)cc1CCN(C)C] |
| | 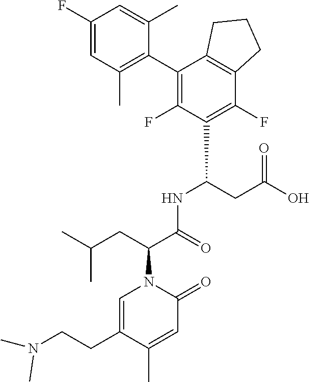 [SMILES: Cc1cc(=O)n([C@@H](CC(C)C)C(=O)N[C@@H](CC(=O)O)c2c(F)c3c(c(-c4c(C)cc(F)cc4C)c2F)CCC3)cc1CCN(C)C] |
| | 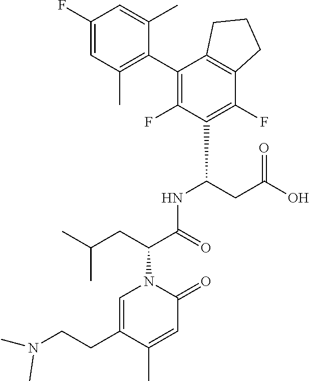 [SMILES: Cc1cc(=O)n([C@H](CC(C)C)C(=O)N[C@@H](CC(=O)O)c2c(F)c3c(c(-c4c(C)cc(F)cc4C)c2F)CCC3)cc1CCN(C)C] |
| | 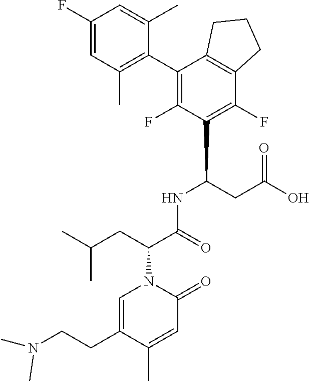 [SMILES: Cc1cc(=O)n([C@H](CC(C)C)C(=O)N[C@H](CC(=O)O)c2c(F)c3c(c(-c4c(C)cc(F)cc4C)c2F)CCC3)cc1CCN(C)C] |
| 59 | 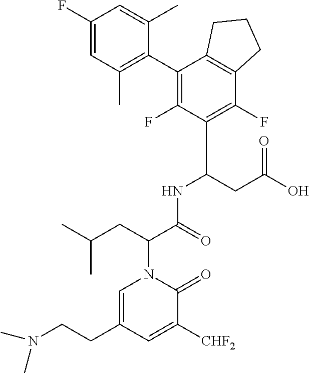 [SMILES: Cc1cc(F)cc(C)c1-c1c(F)c(C(CC(=O)O)NC(=O)C(CC(C)C)n2cc(CCN(C)C)cc(C(F)F)c2=O)c(F)c2c1CCC2] |
| | 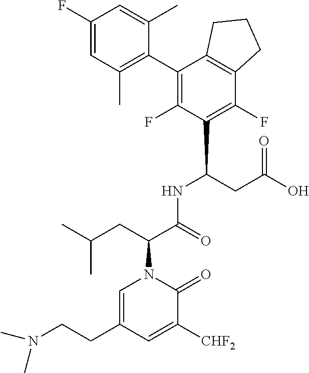 [SMILES: Cc1cc(F)cc(C)c1-c1c(F)c([C@@H](CC(=O)O)NC(=O)[C@H](CC(C)C)n2cc(CCN(C)C)cc(C(F)F)c2=O)c(F)c2c1CCC2] |
| | 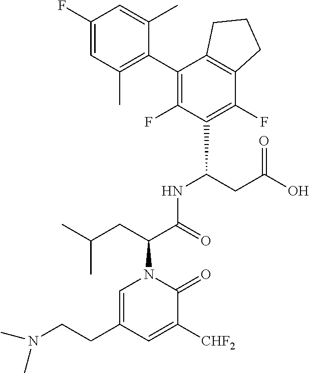 [SMILES: Cc1cc(F)cc(C)c1-c1c(F)c([C@H](CC(=O)O)NC(=O)[C@H](CC(C)C)n2cc(CCN(C)C)cc(C(F)F)c2=O)c(F)c2c1CCC2] |
| | 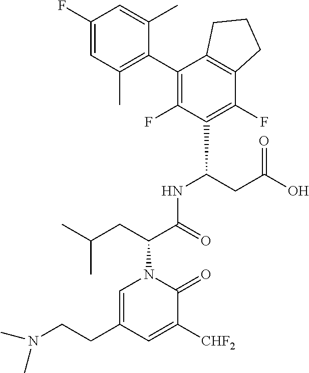 [SMILES: Cc1cc(F)cc(C)c1-c1c(F)c([C@H](CC(=O)O)NC(=O)[C@@H](CC(C)C)n2cc(CCN(C)C)cc(C(F)F)c2=O)c(F)c2c1CCC2] |
| | 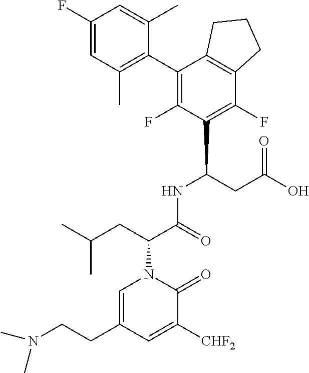 [SMILES: Cc1cc(F)cc(C)c1-c1c(F)c([C@@H](CC(=O)O)NC(=O)[C@@H](CC(C)C)n2cc(CCN(C)C)cc(C(F)F)c2=O)c(F)c2c1CCC2] |
| 60 | 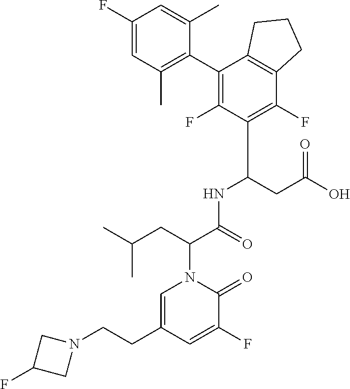 [SMILES: Cc1cc(F)cc(C)c1-c1c(F)c(C(CC(=O)O)NC(=O)C(CC(C)C)n2cc(CCN3CC(F)C3)cc(F)c2=O)c(F)c2c1CCC2] |
| | 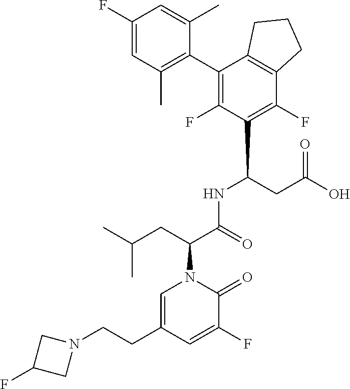 [SMILES: Cc1cc(F)cc(C)c1-c1c(F)c([C@@H](CC(=O)O)NC(=O)[C@H](CC(C)C)n2cc(CCN3CC(F)C3)cc(F)c2=O)c(F)c2c1CCC2] |
| | 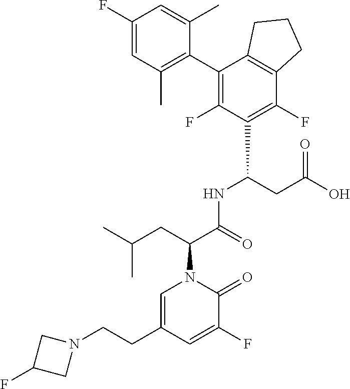 [SMILES: Cc1cc(F)cc(C)c1-c1c(F)c([C@H](CC(=O)O)NC(=O)[C@H](CC(C)C)n2cc(CCN3CC(F)C3)cc(F)c2=O)c(F)c2c1CCC2] |
| | 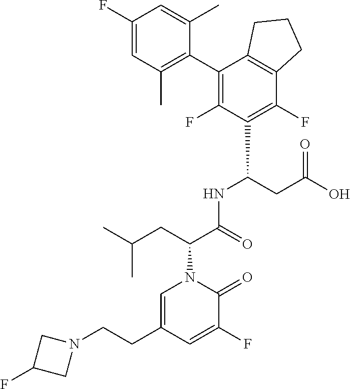 [SMILES: Cc1cc(F)cc(C)c1-c1c(F)c([C@H](CC(=O)O)NC(=O)[C@@H](CC(C)C)n2cc(CCN3CC(F)C3)cc(F)c2=O)c(F)c2c1CCC2] |
| | 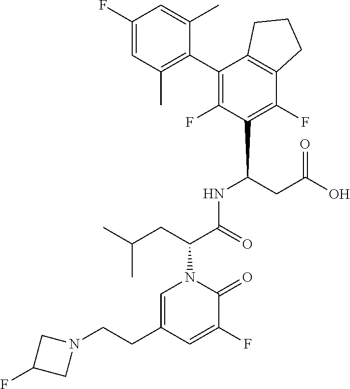 [SMILES: Cc1cc(F)cc(C)c1-c1c(F)c([C@@H](CC(=O)O)NC(=O)[C@@H](CC(C)C)n2cc(CCN3CC(F)C3)cc(F)c2=O)c(F)c2c1CCC2] |
| 61 | 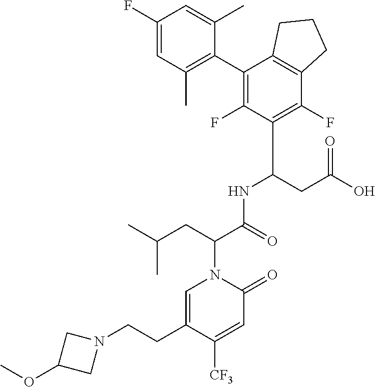 [SMILES: COC1CN(CCc2cn(C(CC(C)C)C(=O)NC(CC(=O)O)c3c(F)c4c(c(-c5c(C)cc(F)cc5C)c3F)CCC4)c(=O)cc2C(F)(F)F)C1] |
| | 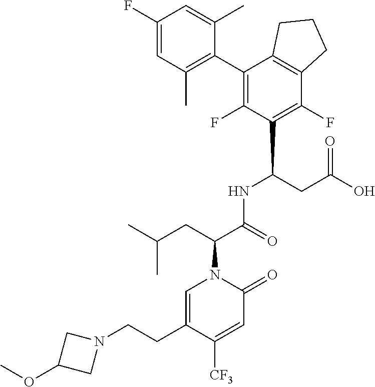 [SMILES: COC1CN(CCc2cn([C@@H](CC(C)C)C(=O)N[C@H](CC(=O)O)c3c(F)c4c(c(-c5c(C)cc(F)cc5C)c3F)CCC4)c(=O)cc2C(F)(F)F)C1] |
| | 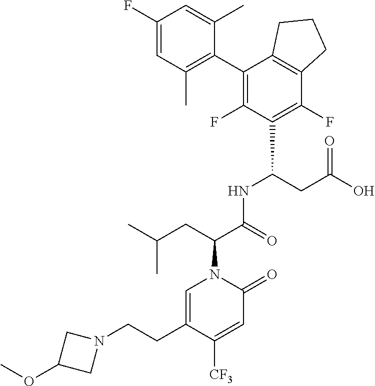 [SMILES: COC1CN(CCc2cn([C@@H](CC(C)C)C(=O)N[C@@H](CC(=O)O)c3c(F)c4c(c(-c5c(C)cc(F)cc5C)c3F)CCC4)c(=O)cc2C(F)(F)F)C1] |
| | 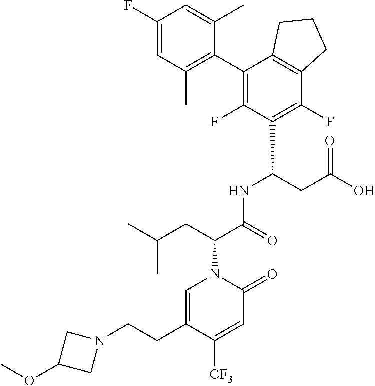 [SMILES: COC1CN(CCc2cn([C@H](CC(C)C)C(=O)N[C@@H](CC(=O)O)c3c(F)c4c(c(-c5c(C)cc(F)cc5C)c3F)CCC4)c(=O)cc2C(F)(F)F)C1] |
| | 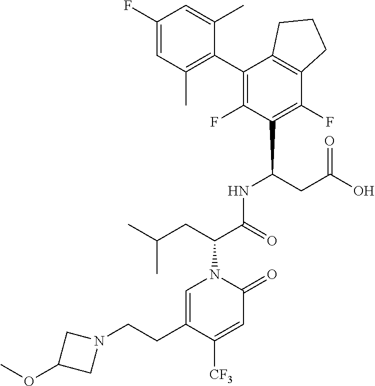 [SMILES: COC1CN(CCc2cn([C@H](CC(C)C)C(=O)N[C@H](CC(=O)O)c3c(F)c4c(c(-c5c(C)cc(F)cc5C)c3F)CCC4)c(=O)cc2C(F)(F)F)C1] |
| 62 | 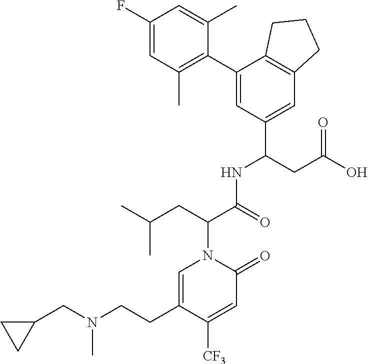 [SMILES: Cc1cc(F)cc(C)c1-c1cc(C(CC(=O)O)NC(=O)C(CC(C)C)n2cc(CCN(C)CC3CC3)c(C(F)(F)F)cc2=O)cc2c1CCC2] |
| | 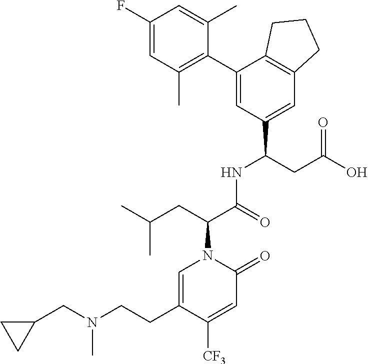 [SMILES: Cc1cc(F)cc(C)c1-c1cc([C@@H](CC(=O)O)NC(=O)[C@H](CC(C)C)n2cc(CCN(C)CC3CC3)c(C(F)(F)F)cc2=O)cc2c1CCC2] |
| | 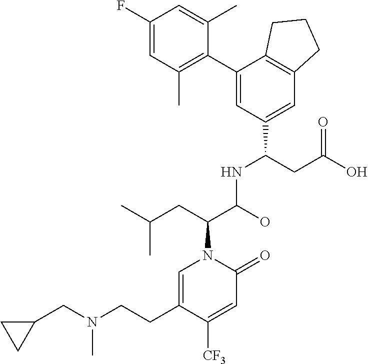 [SMILES: Cc1cc(F)cc(C)c1-c1cc([C@H](CC(=O)O)NC(O)[C@H](CC(C)C)n2cc(CCN(C)CC3CC3)c(C(F)(F)F)cc2=O)cc2c1CCC2] |
| | 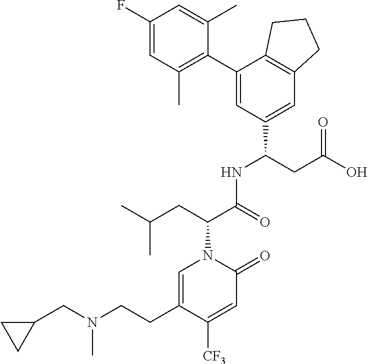 [SMILES: Cc1cc(F)cc(C)c1-c1cc([C@H](CC(=O)O)NC(=O)[C@@H](CC(C)C)n2cc(CCN(C)CC3CC3)c(C(F)(F)F)cc2=O)cc2c1CCC2] |
| | 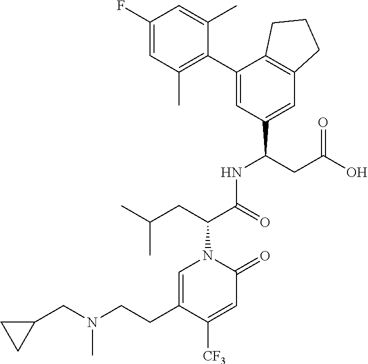 [SMILES: Cc1cc(F)cc(C)c1-c1cc([C@@H](CC(=O)O)NC(=O)[C@@H](CC(C)C)n2cc(CCN(C)CC3CC3)c(C(F)(F)F)cc2=O)cc2c1CCC2] |
| 63 | 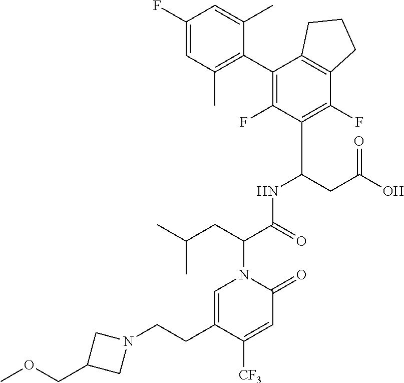 [SMILES: COCC1CN(CCc2cn(C(CC(C)C)C(=O)NC(CC(=O)O)c3c(F)c4c(c(-c5c(C)cc(F)cc5C)c3F)CCC4)c(=O)cc2C(F)(F)F)C1] |
| | 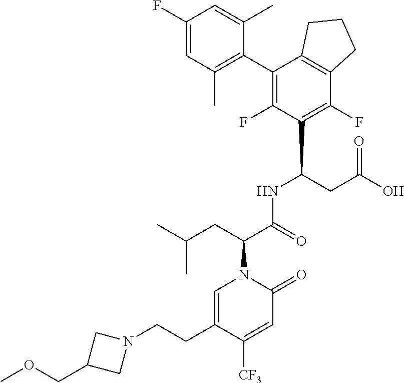 [SMILES: COCC1CN(CCc2cn([C@@H](CC(C)C)C(=O)N[C@H](CC(=O)O)c3c(F)c4c(c(-c5c(C)cc(F)cc5C)c3F)CCC4)c(=O)cc2C(F)(F)F)C1] |
| | 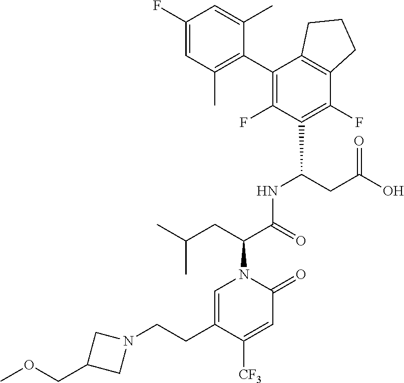 [SMILES: COCC1CN(CCc2cn([C@@H](CC(C)C)C(=O)N[C@@H](CC(=O)O)c3c(F)c4c(c(-c5c(C)cc(F)cc5C)c3F)CCC4)c(=O)cc2C(F)(F)F)C1] |
| | 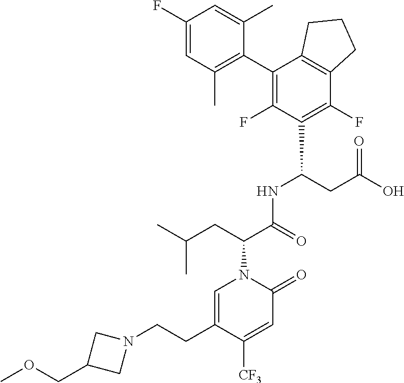 [SMILES: COCC1CN(CCc2cn([C@H](CC(C)C)C(=O)N[C@@H](CC(=O)O)c3c(F)c4c(c(-c5c(C)cc(F)cc5C)c3F)CCC4)c(=O)cc2C(F)(F)F)C1] |
| | 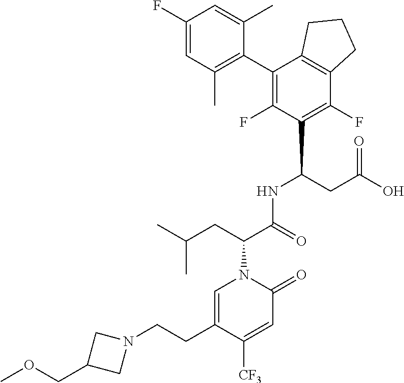 [SMILES: COCC1CN(CCc2cn([C@H](CC(C)C)C(=O)N[C@H](CC(=O)O)c3c(F)c4c(c(-c5c(C)cc(F)cc5C)c3F)CCC4)c(=O)cc2C(F)(F)F)C1] |
| 64 | 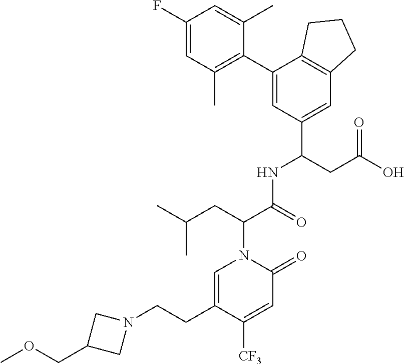 [SMILES: COCC1CN(CCc2cn(C(CC(C)C)C(=O)NC(CC(=O)O)c3cc4c(c(-c5c(C)cc(F)cc5C)c3)CCC4)c(=O)cc2C(F)(F)F)C1] |
| | 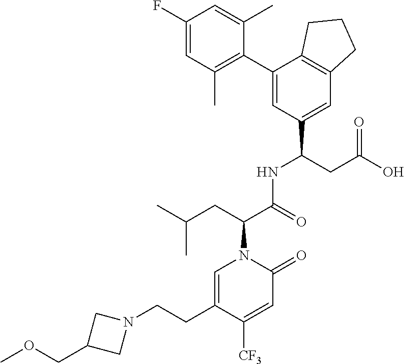 [SMILES: COCC1CN(CCc2cn([C@@H](CC(C)C)C(=O)N[C@H](CC(=O)O)c3cc4c(c(-c5c(C)cc(F)cc5C)c3)CCC4)c(=O)cc2C(F)(F)F)C1] |
| | 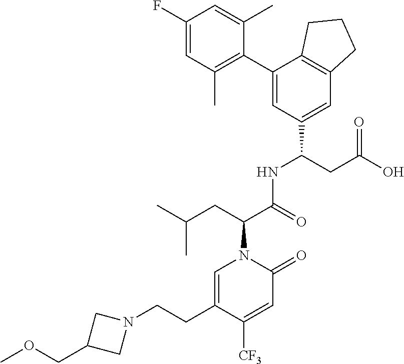 [SMILES: COCC1CN(CCc2cn([C@@H](CC(C)C)C(=O)N[C@@H](CC(=O)O)c3cc4c(c(-c5c(C)cc(F)cc5C)c3)CCC4)c(=O)cc2C(F)(F)F)C1] |
| | 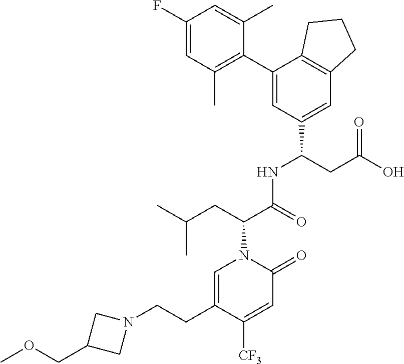 [SMILES: COCC1CN(CCc2cn([C@H](CC(C)C)C(=O)N[C@@H](CC(=O)O)c3cc4c(c(-c5c(C)cc(F)cc5C)c3)CCC4)c(=O)cc2C(F)(F)F)C1] |
| | 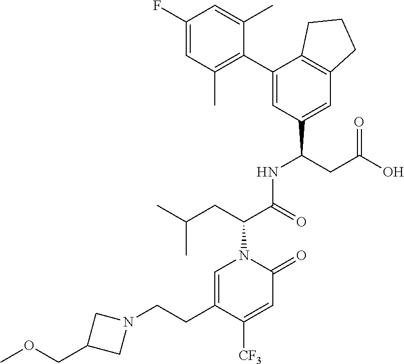 [SMILES: COCC1CN(CCc2cn([C@H](CC(C)C)C(=O)N[C@H](CC(=O)O)c3cc4c(c(-c5c(C)cc(F)cc5C)c3)CCC4)c(=O)cc2C(F)(F)F)C1] |
| 65 | 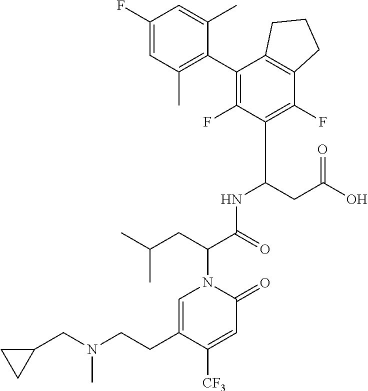 [SMILES: Cc1cc(F)cc(C)c1-c1c(F)c(C(CC(=O)O)NC(=O)C(CC(C)C)n2cc(CCN(C)CC3CC3)c(C(F)(F)F)cc2=O)c(F)c2c1CCC2] |
| | 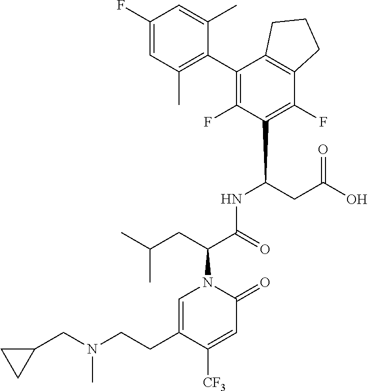 [SMILES: Cc1cc(F)cc(C)c1-c1c(F)c([C@@H](CC(=O)O)NC(=O)[C@H](CC(C)C)n2cc(CCN(C)CC3CC3)c(C(F)(F)F)cc2=O)c(F)c2c1CCC2] |
| | 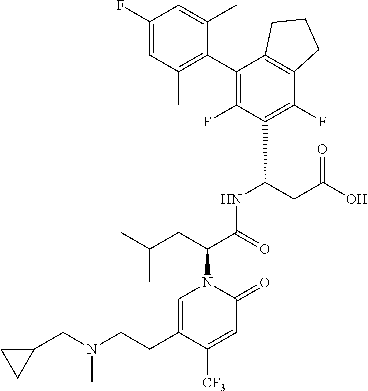 [SMILES: Cc1cc(F)cc(C)c1-c1c(F)c([C@H](CC(=O)O)NC(=O)[C@H](CC(C)C)n2cc(CCN(C)CC3CC3)c(C(F)(F)F)cc2=O)c(F)c2c1CCC2] |
| | 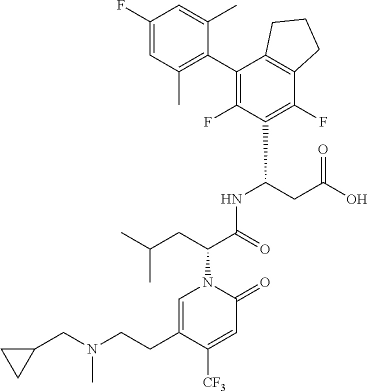 [SMILES: Cc1cc(F)cc(C)c1-c1c(F)c([C@H](CC(=O)O)NC(=O)[C@@H](CC(C)C)n2cc(CCN(C)CC3CC3)c(C(F)(F)F)cc2=O)c(F)c2c1CCC2] |
| | 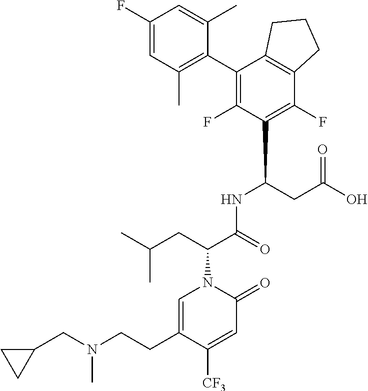 [SMILES: Cc1cc(F)cc(C)c1-c1c(F)c([C@@H](CC(=O)O)NC(=O)[C@@H](CC(C)C)n2cc(CCN(C)CC3CC3)c(C(F)(F)F)cc2=O)c(F)c2c1CCC2] |
| 66 | 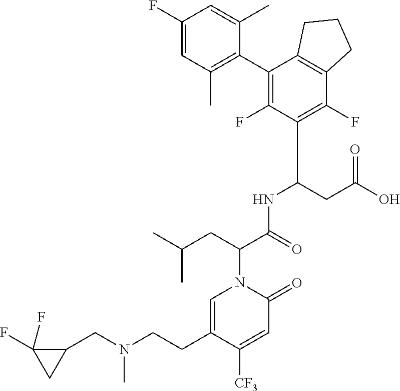 [SMILES: Cc1cc(F)cc(C)c1-c1c(F)c(C(CC(=O)O)NC(=O)C(CC(C)C)n2cc(CCN(C)CC3CC3(F)F)c(C(F)(F)F)cc2=O)c(F)c2c1CCC2] |
| | 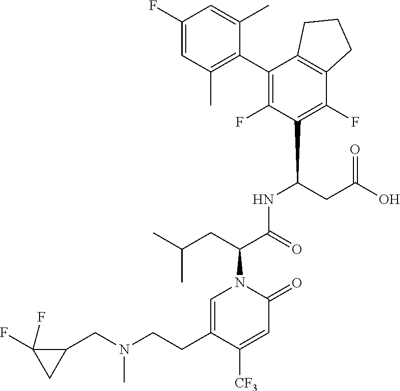 [SMILES: Cc1cc(F)cc(C)c1-c1c(F)c([C@@H](CC(=O)O)NC(=O)[C@H](CC(C)C)n2cc(CCN(C)CC3CC3(F)F)c(C(F)(F)F)cc2=O)c(F)c2c1CCC2] |
| | 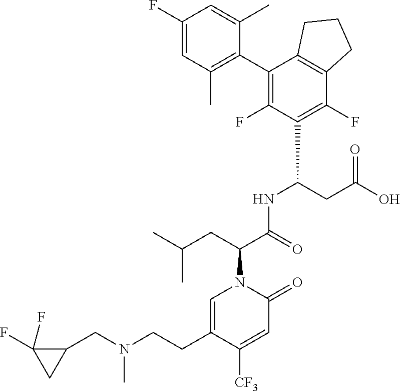 [SMILES: Cc1cc(F)cc(C)c1-c1c(F)c([C@H](CC(=O)O)NC(=O)[C@H](CC(C)C)n2cc(CCN(C)CC3CC3(F)F)c(C(F)(F)F)cc2=O)c(F)c2c1CCC2] |
| | 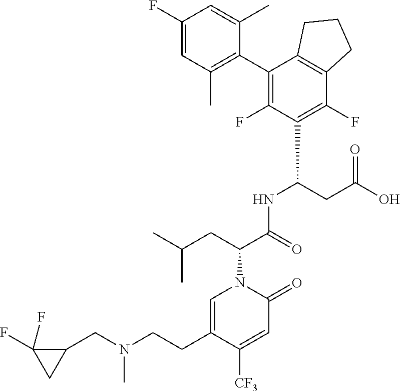 [SMILES: Cc1cc(F)cc(C)c1-c1c(F)c([C@H](CC(=O)O)NC(=O)[C@@H](CC(C)C)n2cc(CCN(C)CC3CC3(F)F)c(C(F)(F)F)cc2=O)c(F)c2c1CCC2] |
| | 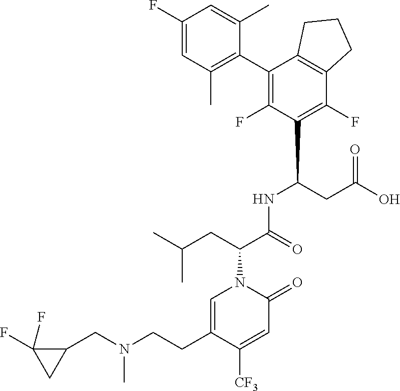 [SMILES: Cc1cc(F)cc(C)c1-c1c(F)c([C@@H](CC(=O)O)NC(=O)[C@@H](CC(C)C)n2cc(CCN(C)CC3CC3(F)F)c(C(F)(F)F)cc2=O)c(F)c2c1CCC2] |
| 67 | 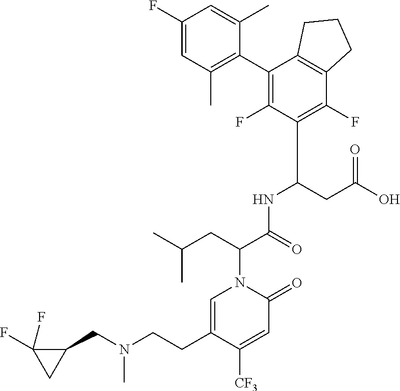 [SMILES: Cc1cc(F)cc(C)c1-c1c(F)c(C(CC(=O)O)NC(=O)C(CC(C)C)n2cc(CCN(C)C[C@H]3CC3(F)F)c(C(F)(F)F)cc2=O)c(F)c2c1CCC2] |
| | 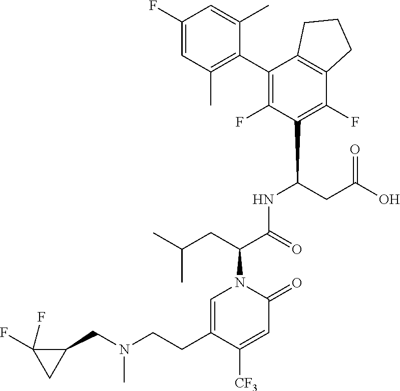 [SMILES: Cc1cc(F)cc(C)c1-c1c(F)c([C@@H](CC(=O)O)NC(=O)[C@H](CC(C)C)n2cc(CCN(C)C[C@H]3CC3(F)F)c(C(F)(F)F)cc2=O)c(F)c2c1CCC2] |
| | 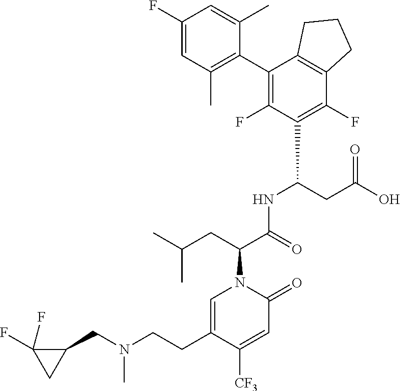 [SMILES: Cc1cc(F)cc(C)c1-c1c(F)c([C@H](CC(=O)O)NC(=O)[C@H](CC(C)C)n2cc(CCN(C)C[C@H]3CC3(F)F)c(C(F)(F)F)cc2=O)c(F)c2c1CCC2] |
| | 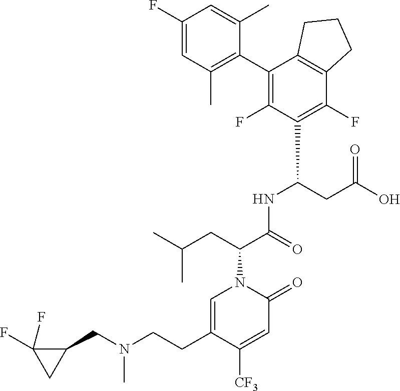 [SMILES: Cc1cc(F)cc(C)c1-c1c(F)c([C@H](CC(=O)O)NC(=O)[C@@H](CC(C)C)n2cc(CCN(C)C[C@H]3CC3(F)F)c(C(F)(F)F)cc2=O)c(F)c2c1CCC2] |
| | 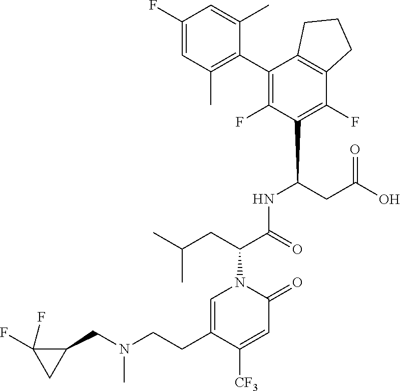 [SMILES: Cc1cc(F)cc(C)c1-c1c(F)c([C@@H](CC(=O)O)NC(=O)[C@@H](CC(C)C)n2cc(CCN(C)C[C@H]3CC3(F)F)c(C(F)(F)F)cc2=O)c(F)c2c1CCC2] |
| 68 | 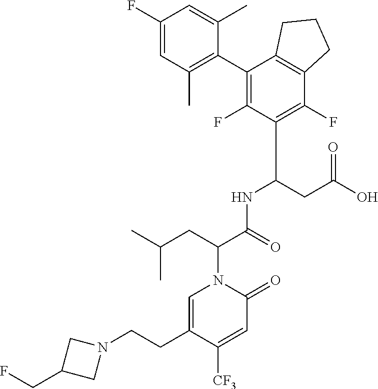 [SMILES: Cc1cc(F)cc(C)c1-c1c(F)c(C(CC(=O)O)NC(=O)C(CC(C)C)n2cc(CCN3CC(CF)C3)c(C(F)(F)F)cc2=O)c(F)c2c1CCC2] |
| | 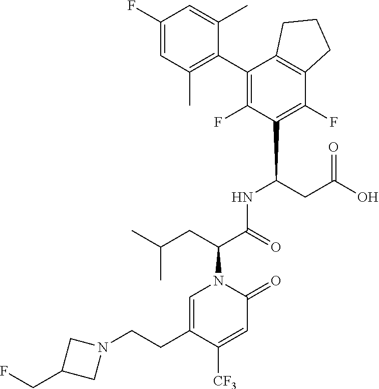 [SMILES: Cc1cc(F)cc(C)c1-c1c(F)c([C@@H](CC(=O)O)NC(=O)[C@H](CC(C)C)n2cc(CCN3CC(CF)C3)c(C(F)(F)F)cc2=O)c(F)c2c1CCC2] |
| | 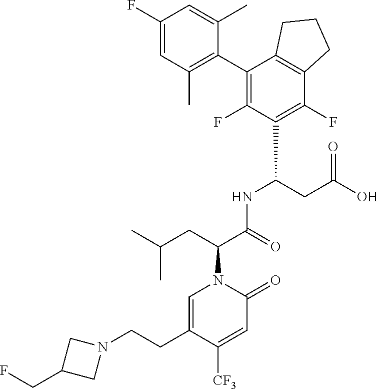 [SMILES: Cc1cc(F)cc(C)c1-c1c(F)c([C@H](CC(=O)O)NC(=O)[C@H](CC(C)C)n2cc(CCN3CC(CF)C3)c(C(F)(F)F)cc2=O)c(F)c2c1CCC2] |
| | 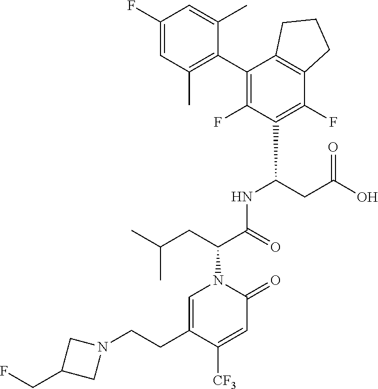 [SMILES: Cc1cc(F)cc(C)c1-c1c(F)c([C@H](CC(=O)O)NC(=O)[C@@H](CC(C)C)n2cc(CCN3CC(CF)C3)c(C(F)(F)F)cc2=O)c(F)c2c1CCC2] |
| | 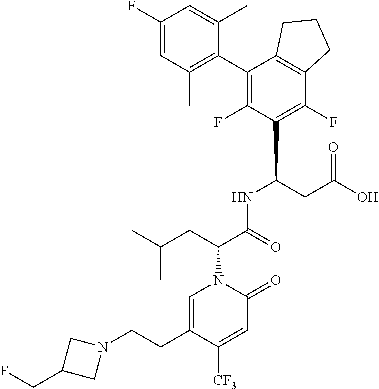 [SMILES: Cc1cc(F)cc(C)c1-c1c(F)c([C@@H](CC(=O)O)NC(=O)[C@@H](CC(C)C)n2cc(CCN3CC(CF)C3)c(C(F)(F)F)cc2=O)c(F)c2c1CCC2] |
| 69 | 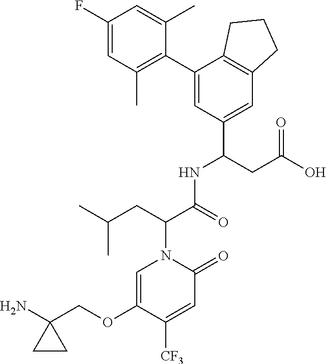 [SMILES: Cc1cc(F)cc(C)c1-c1cc(C(CC(=O)O)NC(=O)C(CC(C)C)n2cc(OCC3(N)CC3)c(C(F)(F)F)cc2=O)cc2c1CCC2] |
| | 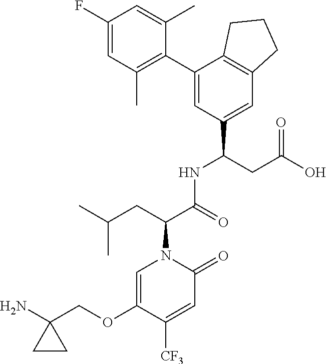 [SMILES: Cc1cc(F)cc(C)c1-c1cc([C@@H](CC(=O)O)NC(=O)[C@H](CC(C)C)n2cc(OCC3(N)CC3)c(C(F)(F)F)cc2=O)cc2c1CCC2] |
| | 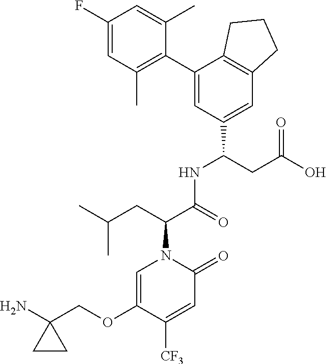 [SMILES: Cc1cc(F)cc(C)c1-c1cc([C@H](CC(=O)O)NC(=O)[C@H](CC(C)C)n2cc(OCC3(N)CC3)c(C(F)(F)F)cc2=O)cc2c1CCC2] |
| | 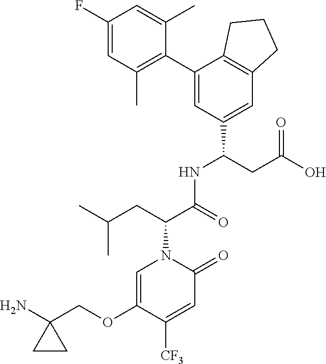 [SMILES: Cc1cc(F)cc(C)c1-c1cc([C@H](CC(=O)O)NC(=O)[C@@H](CC(C)C)n2cc(OCC3(N)CC3)c(C(F)(F)F)cc2=O)cc2c1CCC2] |
| | 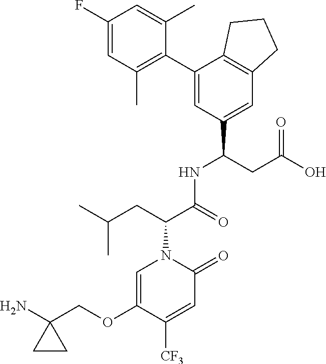 [SMILES: Cc1cc(F)cc(C)c1-c1cc([C@@H](CC(=O)O)NC(=O)[C@@H](CC(C)C)n2cc(OCC3(N)CC3)c(C(F)(F)F)cc2=O)cc2c1CCC2] |
| 70 | 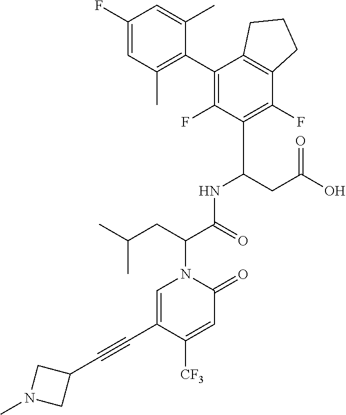 [SMILES: Cc1cc(F)cc(C)c1-c1c(F)c(C(CC(=O)O)NC(=O)C(CC(C)C)n2cc(C#CC3CN(C)C3)c(C(F)(F)F)cc2=O)c(F)c2c1CCC2] |
| | 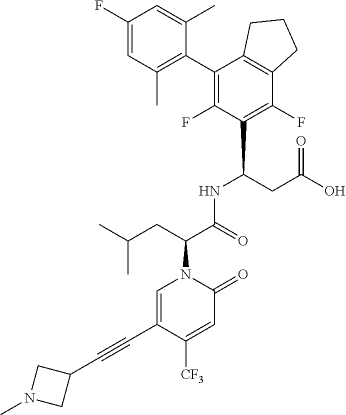 [SMILES: Cc1cc(F)cc(C)c1-c1c(F)c([C@@H](CC(=O)O)NC(=O)[C@H](CC(C)C)n2cc(C#CC3CN(C)C3)c(C(F)(F)F)cc2=O)c(F)c2c1CCC2] |
| | 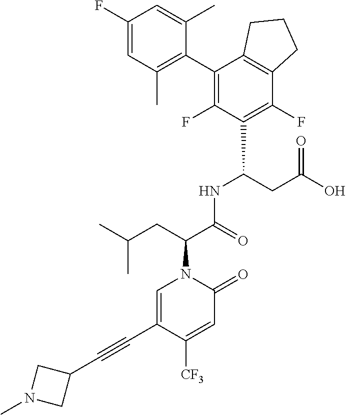 [SMILES: Cc1cc(F)cc(C)c1-c1c(F)c([C@H](CC(=O)O)NC(=O)[C@H](CC(C)C)n2cc(C#CC3CN(C)C3)c(C(F)(F)F)cc2=O)c(F)c2c1CCC2] |
| | 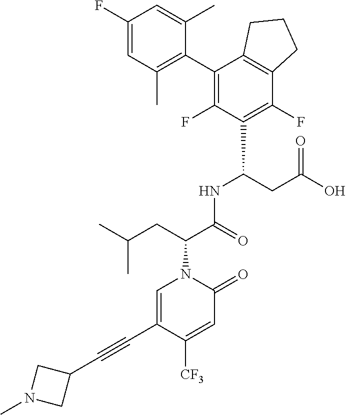 [SMILES: Cc1cc(F)cc(C)c1-c1c(F)c([C@H](CC(=O)O)NC(=O)[C@@H](CC(C)C)n2cc(C#CC3CN(C)C3)c(C(F)(F)F)cc2=O)c(F)c2c1CCC2] |
| | 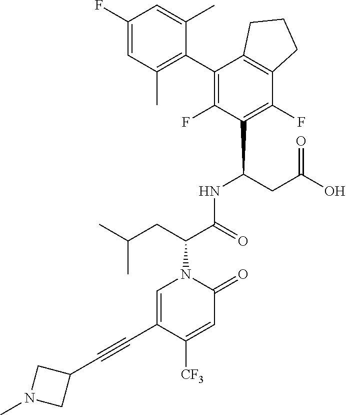 [SMILES: Cc1cc(F)cc(C)c1-c1c(F)c([C@@H](CC(=O)O)NC(=O)[C@@H](CC(C)C)n2cc(C#CC3CN(C)C3)c(C(F)(F)F)cc2=O)c(F)c2c1CCC2] |
| 71 | 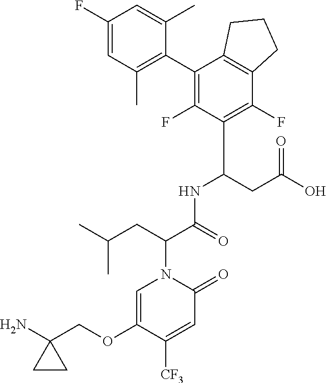 [SMILES: Cc1cc(F)cc(C)c1-c1c(F)c(C(CC(=O)O)NC(=O)C(CC(C)C)n2cc(OCC3(N)CC3)c(C(F)(F)F)cc2=O)c(F)c2c1CCC2] |
| | 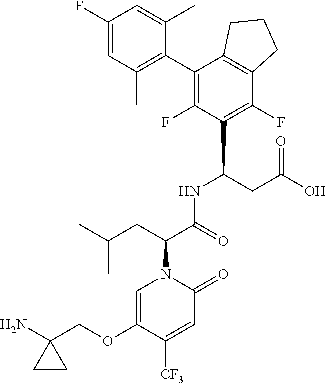 [SMILES: Cc1cc(F)cc(C)c1-c1c(F)c([C@@H](CC(=O)O)NC(=O)[C@H](CC(C)C)n2cc(OCC3(N)CC3)c(C(F)(F)F)cc2=O)c(F)c2c1CCC2] |
| | 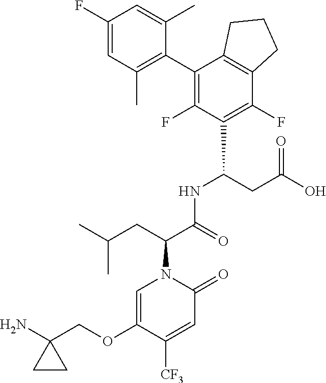 [SMILES: Cc1cc(F)cc(C)c1-c1c(F)c([C@H](CC(=O)O)NC(=O)[C@H](CC(C)C)n2cc(OCC3(N)CC3)c(C(F)(F)F)cc2=O)c(F)c2c1CCC2] |
| | 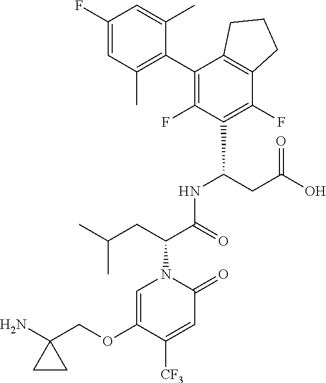 [SMILES: Cc1cc(F)cc(C)c1-c1c(F)c([C@H](CC(=O)O)NC(=O)[C@@H](CC(C)C)n2cc(OCC3(N)CC3)c(C(F)(F)F)cc2=O)c(F)c2c1CCC2] |
| | 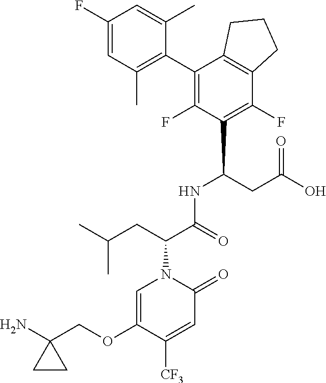 [SMILES: Cc1cc(F)cc(C)c1-c1c(F)c([C@@H](CC(=O)O)NC(=O)[C@@H](CC(C)C)n2cc(OCC3(N)CC3)c(C(F)(F)F)cc2=O)c(F)c2c1CCC2] |
| 72 | 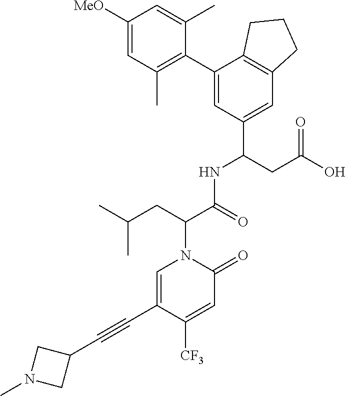 [SMILES: COc1cc(C)c(-c2cc(C(CC(=O)O)NC(=O)C(CC(C)C)n3cc(C#CC4CN(C)C4)c(C(F)(F)F)cc3=O)cc3c2CCC3)c(C)c1] |
| | 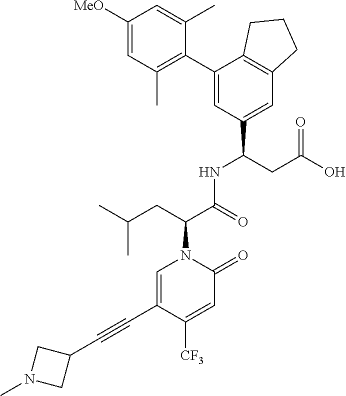 [SMILES: COc1cc(C)c(-c2cc([C@@H](CC(=O)O)NC(=O)[C@H](CC(C)C)n3cc(C#CC4CN(C)C4)c(C(F)(F)F)cc3=O)cc3c2CCC3)c(C)c1] |
| | 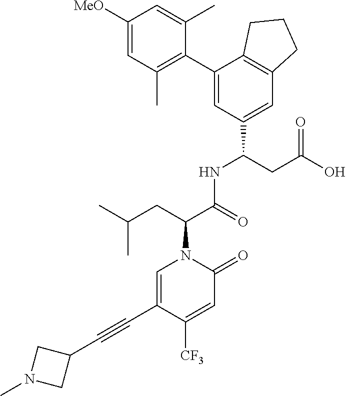 [SMILES: COc1cc(C)c(-c2cc([C@H](CC(=O)O)NC(=O)[C@H](CC(C)C)n3cc(C#CC4CN(C)C4)c(C(F)(F)F)cc3=O)cc3c2CCC3)c(C)c1] |
| | 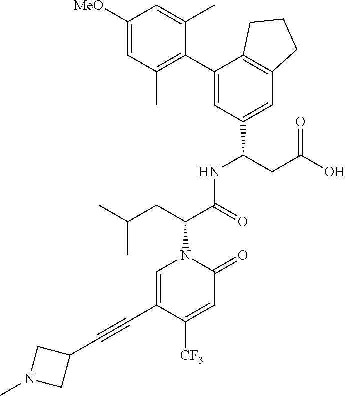 [SMILES: COc1cc(C)c(-c2cc([C@H](CC(=O)O)NC(=O)[C@@H](CC(C)C)n3cc(C#CC4CN(C)C4)c(C(F)(F)F)cc3=O)cc3c2CCC3)c(C)c1] |
| | 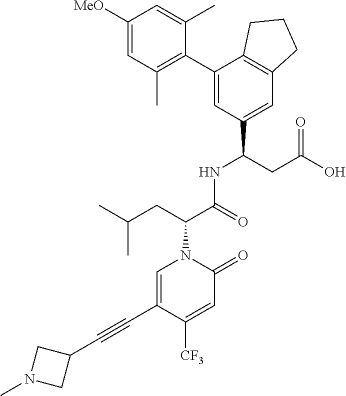 [SMILES: COc1cc(C)c(-c2cc([C@@H](CC(=O)O)NC(=O)[C@@H](CC(C)C)n3cc(C#CC4CN(C)C4)c(C(F)(F)F)cc3=O)cc3c2CCC3)c(C)c1] |
| 73 | 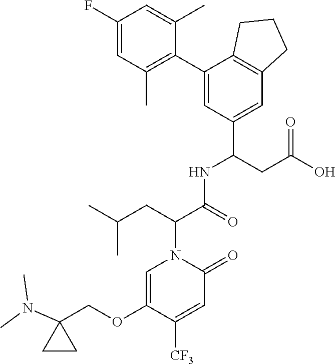 [SMILES: Cc1cc(F)cc(C)c1-c1cc(C(CC(=O)O)NC(=O)C(CC(C)C)n2cc(OCC3(N(C)C)CC3)c(C(F)(F)F)cc2=O)cc2c1CCC2] |
| | 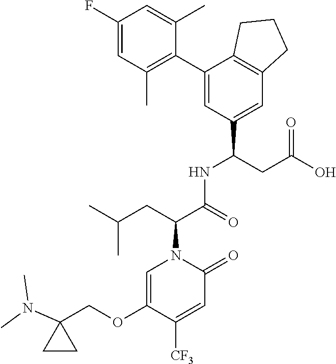 [SMILES: Cc1cc(F)cc(C)c1-c1cc([C@@H](CC(=O)O)NC(=O)[C@H](CC(C)C)n2cc(OCC3(N(C)C)CC3)c(C(F)(F)F)cc2=O)cc2c1CCC2] |
| | 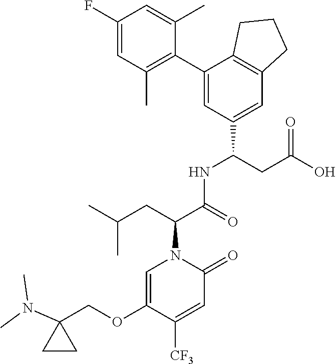 [SMILES: Cc1cc(F)cc(C)c1-c1cc([C@H](CC(=O)O)NC(=O)[C@H](CC(C)C)n2cc(OCC3(N(C)C)CC3)c(C(F)(F)F)cc2=O)cc2c1CCC2] |
| | 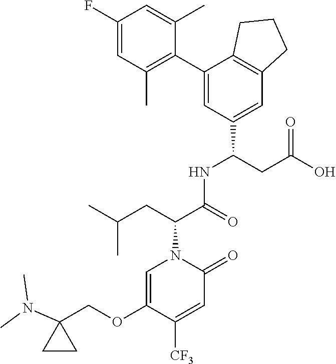 [SMILES: Cc1cc(F)cc(C)c1-c1cc([C@H](CC(=O)O)NC(=O)[C@@H](CC(C)C)n2cc(OCC3(N(C)C)CC3)c(C(F)(F)F)cc2=O)cc2c1CCC2] |
| | 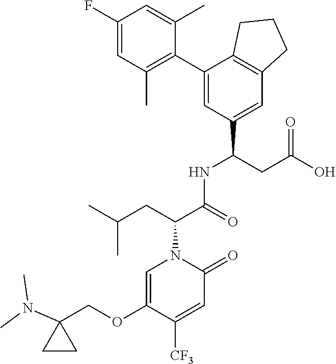 [SMILES: Cc1cc(F)cc(C)c1-c1cc([C@@H](CC(=O)O)NC(=O)[C@@H](CC(C)C)n2cc(OCC3(N(C)C)CC3)c(C(F)(F)F)cc2=O)cc2c1CCC2] |
| 74 | 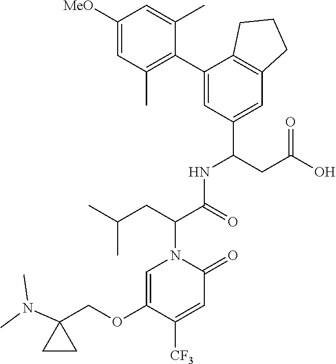 [SMILES: COc1cc(C)c(-c2cc(C(CC(=O)O)NC(=O)C(CC(C)C)n3cc(OCC4(N(C)C)CC4)c(C(F)(F)F)cc3=O)cc3c2CCC3)c(C)c1] |
| | 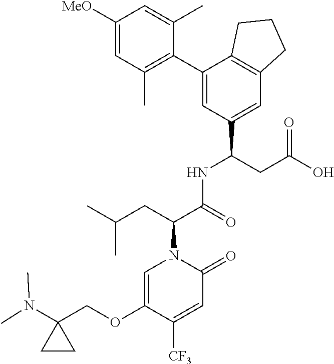 [SMILES: COc1cc(C)c(-c2cc([C@@H](CC(=O)O)NC(=O)[C@H](CC(C)C)n3cc(OCC4(N(C)C)CC4)c(C(F)(F)F)cc3=O)cc3c2CCC3)c(C)c1] |
| | 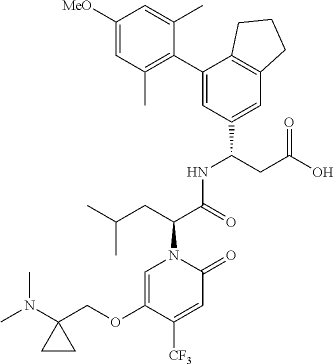 [SMILES: COc1cc(C)c(-c2cc([C@H](CC(=O)O)NC(=O)[C@H](CC(C)C)n3cc(OCC4(N(C)C)CC4)c(C(F)(F)F)cc3=O)cc3c2CCC3)c(C)c1] |
| | 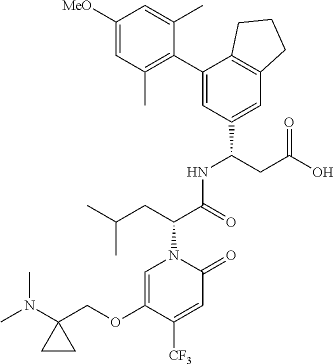 [SMILES: COc1cc(C)c(-c2cc([C@H](CC(=O)O)NC(=O)[C@@H](CC(C)C)n3cc(OCC4(N(C)C)CC4)c(C(F)(F)F)cc3=O)cc3c2CCC3)c(C)c1] |
| | 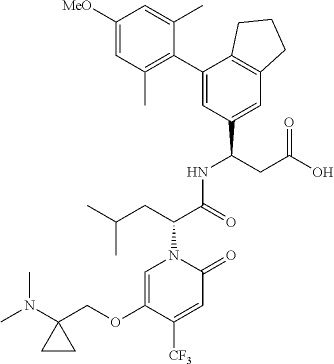 [SMILES: COc1cc(C)c(-c2cc([C@@H](CC(=O)O)NC(=O)[C@@H](CC(C)C)n3cc(OCC4(N(C)C)CC4)c(C(F)(F)F)cc3=O)cc3c2CCC3)c(C)c1] |
| 75 | 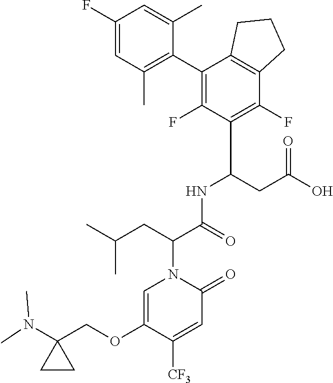 [SMILES: Cc1cc(F)cc(C)c1-c1c(F)c(C(CC(=O)O)NC(=O)C(CC(C)C)n2cc(OCC3(N(C)C)CC3)c(C(F)(F)F)cc2=O)c(F)c2c1CCC2] |
| | 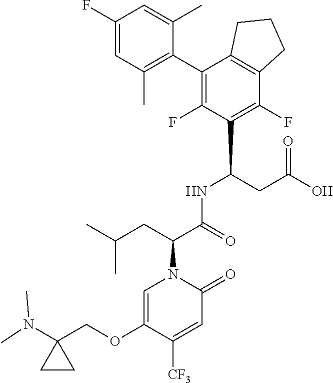 [SMILES: Cc1cc(F)cc(C)c1-c1c(F)c([C@@H](CC(=O)O)NC(=O)[C@H](CC(C)C)n2cc(OCC3(N(C)C)CC3)c(C(F)(F)F)cc2=O)c(F)c2c1CCC2] |
| | 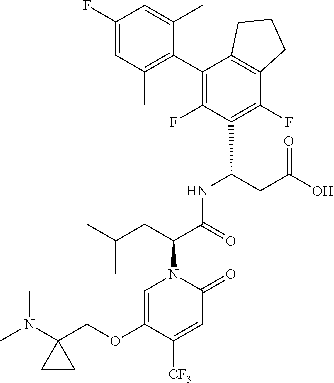 [SMILES: Cc1cc(F)cc(C)c1-c1c(F)c([C@H](CC(=O)O)NC(=O)[C@H](CC(C)C)n2cc(OCC3(N(C)C)CC3)c(C(F)(F)F)cc2=O)c(F)c2c1CCC2] |
| | 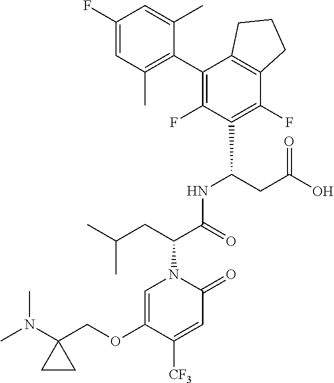 [SMILES: Cc1cc(F)cc(C)c1-c1c(F)c([C@H](CC(=O)O)NC(=O)[C@@H](CC(C)C)n2cc(OCC3(N(C)C)CC3)c(C(F)(F)F)cc2=O)c(F)c2c1CCC2] |
| | 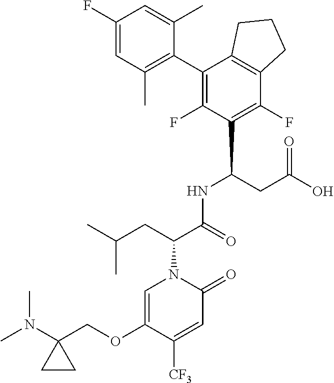 [SMILES: Cc1cc(F)cc(C)c1-c1c(F)c([C@@H](CC(=O)O)NC(=O)[C@@H](CC(C)C)n2cc(OCC3(N(C)C)CC3)c(C(F)(F)F)cc2=O)c(F)c2c1CCC2] |
| 76 | 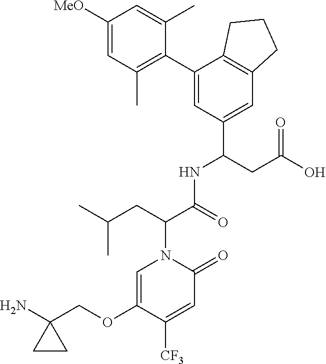 [SMILES: COc1cc(C)c(-c2cc(C(CC(=O)O)NC(=O)C(CC(C)C)n3cc(OCC4(N)CC4)c(C(F)(F)F)cc3=O)cc3c2CCC3)c(C)c1] |
| | 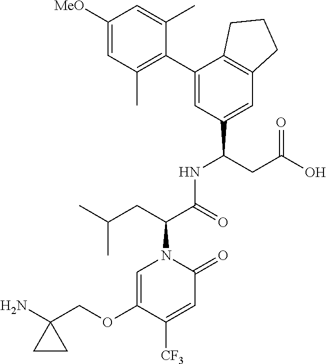 [SMILES: COc1cc(C)c(-c2cc([C@@H](CC(=O)O)NC(=O)[C@H](CC(C)C)n3cc(OCC4(N)CC4)c(C(F)(F)F)cc3=O)cc3c2CCC3)c(C)c1] |
| | 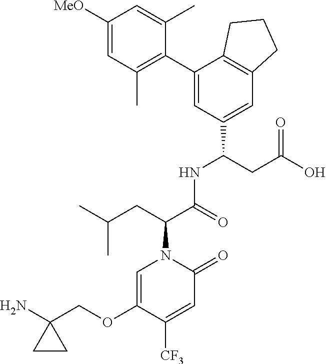 [SMILES: COc1cc(C)c(-c2cc([C@H](CC(=O)O)NC(=O)[C@H](CC(C)C)n3cc(OCC4(N)CC4)c(C(F)(F)F)cc3=O)cc3c2CCC3)c(C)c1] |
| | 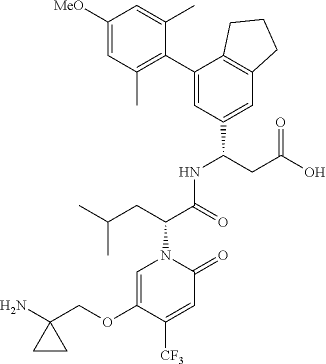 [SMILES: COc1cc(C)c(-c2cc([C@H](CC(=O)O)NC(=O)[C@@H](CC(C)C)n3cc(OCC4(N)CC4)c(C(F)(F)F)cc3=O)cc3c2CCC3)c(C)c1] |
| | 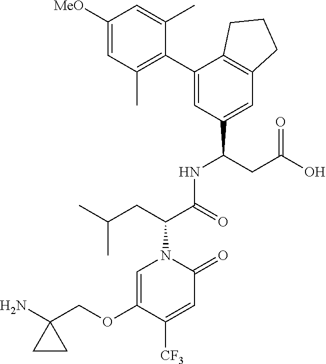 [SMILES: COc1cc(C)c(-c2cc([C@@H](CC(=O)O)NC(=O)[C@@H](CC(C)C)n3cc(OCC4(N)CC4)c(C(F)(F)F)cc3=O)cc3c2CCC3)c(C)c1] |
| 77 | 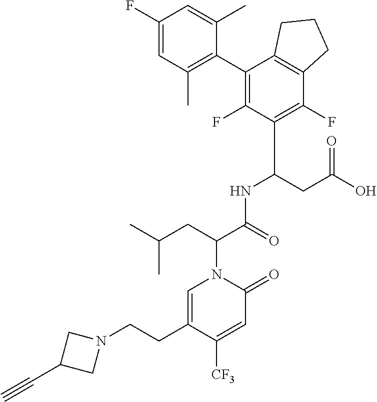 [SMILES: C#CC1CN(CCc2cn(C(CC(C)C)C(=O)NC(CC(=O)O)c3c(F)c4c(c(-c5c(C)cc(F)cc5C)c3F)CCC4)c(=O)cc2C(F)(F)F)C1] |
| | 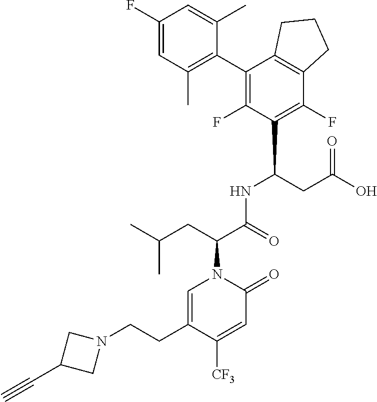 [SMILES: C#CC1CN(CCc2cn([C@@H](CC(C)C)C(=O)N[C@H](CC(=O)O)c3c(F)c4c(c(-c5c(C)cc(F)cc5C)c3F)CCC4)c(=O)cc2C(F)(F)F)C1] |
| | 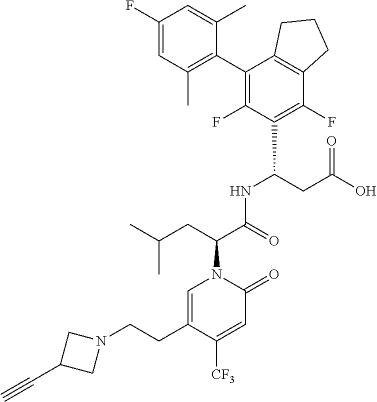 [SMILES: C#CC1CN(CCc2cn([C@@H](CC(C)C)C(=O)N[C@@H](CC(=O)O)c3c(F)c4c(c(-c5c(C)cc(F)cc5C)c3F)CCC4)c(=O)cc2C(F)(F)F)C1] |
| | 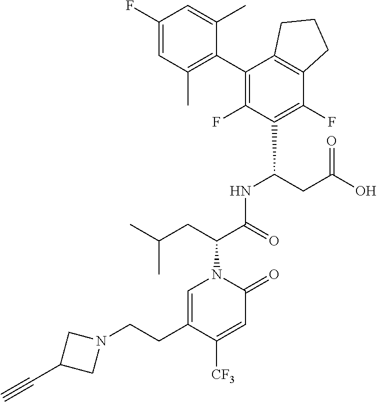 [SMILES: C#CC1CN(CCc2cn([C@H](CC(C)C)C(=O)N[C@@H](CC(=O)O)c3c(F)c4c(c(-c5c(C)cc(F)cc5C)c3F)CCC4)c(=O)cc2C(F)(F)F)C1] |
| | 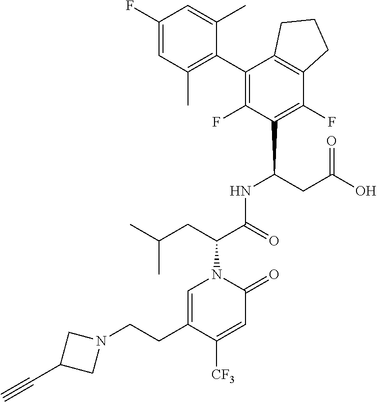 [SMILES: C#CC1CN(CCc2cn([C@H](CC(C)C)C(=O)N[C@H](CC(=O)O)c3c(F)c4c(c(-c5c(C)cc(F)cc5C)c3F)CCC4)c(=O)cc2C(F)(F)F)C1] |
| 78 | 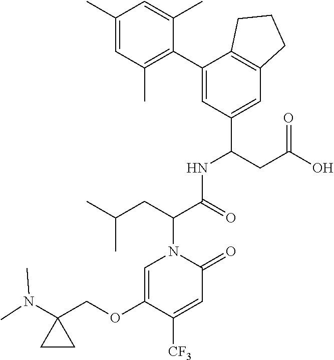 [SMILES: Cc1cc(C)c(-c2cc(C(CC(=O)O)NC(=O)C(CC(C)C)n3cc(OCC4(N(C)C)CC4)c(C(F)(F)F)cc3=O)cc3c2CCC3)c(C)c1] |
| | 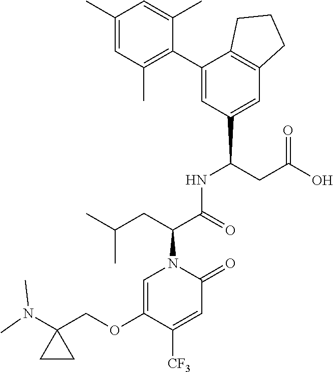 [SMILES: Cc1cc(C)c(-c2cc([C@@H](CC(=O)O)NC(=O)[C@H](CC(C)C)n3cc(OCC4(N(C)C)CC4)c(C(F)(F)F)cc3=O)cc3c2CCC3)c(C)c1] |
| | 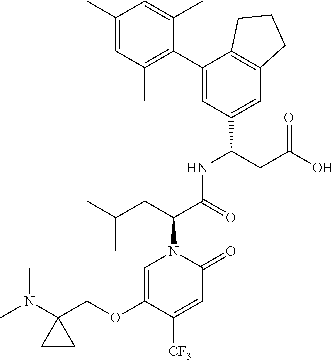 [SMILES: Cc1cc(C)c(-c2cc([C@H](CC(=O)O)NC(=O)[C@H](CC(C)C)n3cc(OCC4(N(C)C)CC4)c(C(F)(F)F)cc3=O)cc3c2CCC3)c(C)c1] |
| | 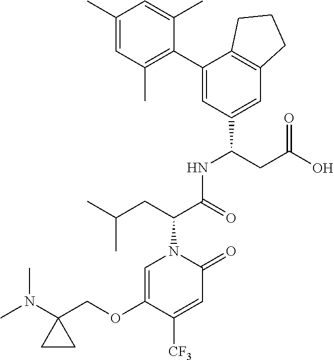 [SMILES: Cc1cc(C)c(-c2cc([C@H](CC(=O)O)NC(=O)[C@@H](CC(C)C)n3cc(OCC4(N(C)C)CC4)c(C(F)(F)F)cc3=O)cc3c2CCC3)c(C)c1] |
| | 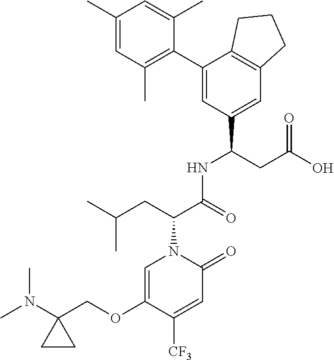 [SMILES: Cc1cc(C)c(-c2cc([C@@H](CC(=O)O)NC(=O)[C@@H](CC(C)C)n3cc(OCC4(N(C)C)CC4)c(C(F)(F)F)cc3=O)cc3c2CCC3)c(C)c1] |
| 79 | 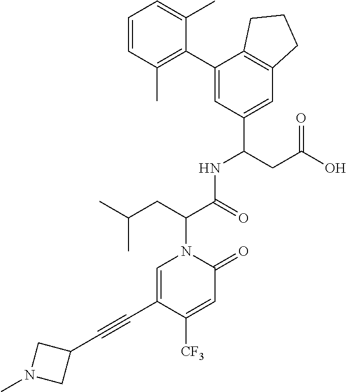 [SMILES: Cc1cccc(C)c1-c1cc(C(CC(=O)O)NC(=O)C(CC(C)C)n2cc(C#CC3CN(C)C3)c(C(F)(F)F)cc2=O)cc2c1CCC2] |
| | 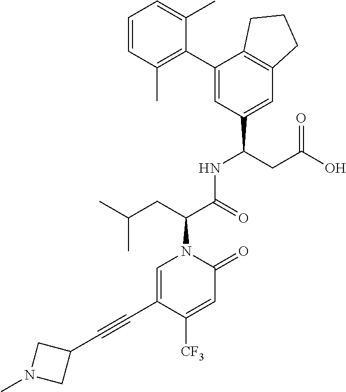 [SMILES: Cc1cccc(C)c1-c1cc([C@@H](CC(=O)O)NC(=O)[C@H](CC(C)C)n2cc(C#CC3CN(C)C3)c(C(F)(F)F)cc2=O)cc2c1CCC2] |
| | 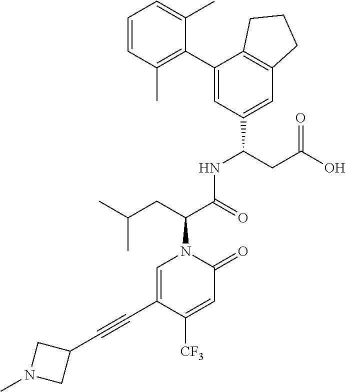 [SMILES: Cc1cccc(C)c1-c1cc([C@H](CC(=O)O)NC(=O)[C@H](CC(C)C)n2cc(C#CC3CN(C)C3)c(C(F)(F)F)cc2=O)cc2c1CCC2] |
| | 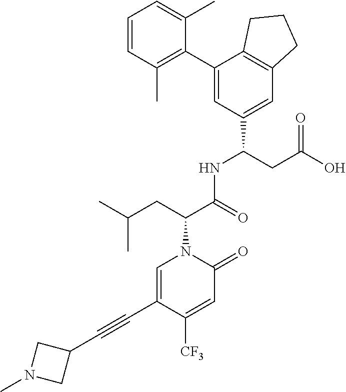 [SMILES: Cc1cccc(C)c1-c1cc([C@H](CC(=O)O)NC(=O)[C@@H](CC(C)C)n2cc(C#CC3CN(C)C3)c(C(F)(F)F)cc2=O)cc2c1CCC2] |
| | 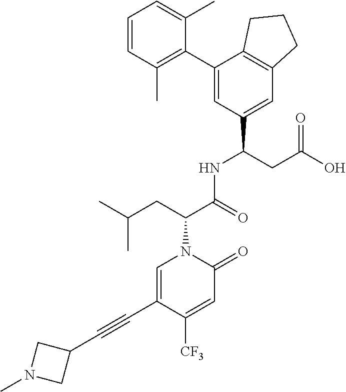 [SMILES: Cc1cccc(C)c1-c1cc([C@@H](CC(=O)O)NC(=O)[C@@H](CC(C)C)n2cc(C#CC3CN(C)C3)c(C(F)(F)F)cc2=O)cc2c1CCC2] |
| 80 | 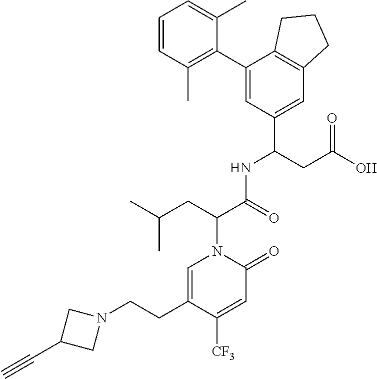 [SMILES: C#CC1CN(CCc2cn(C(CC(C)C)C(=O)NC(CC(=O)O)c3cc4c(c(-c5c(C)cccc5C)c3)CCC4)c(=O)cc2C(F)(F)F)C1] |
| | 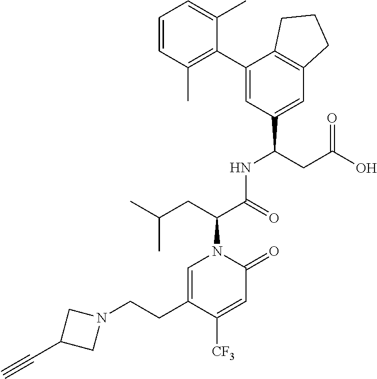 [SMILES: C#CC1CN(CCc2cn([C@@H](CC(C)C)C(=O)N[C@H](CC(=O)O)c3cc4c(c(-c5c(C)cccc5C)c3)CCC4)c(=O)cc2C(F)(F)F)C1] |
| | 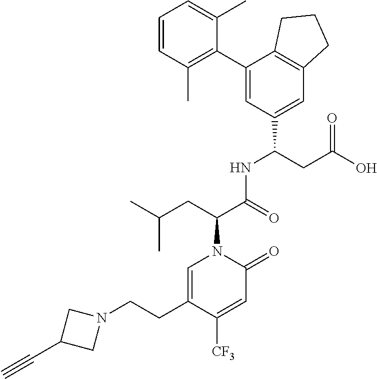 [SMILES: C#CC1CN(CCc2cn([C@@H](CC(C)C)C(=O)N[C@@H](CC(=O)O)c3cc4c(c(-c5c(C)cccc5C)c3)CCC4)c(=O)cc2C(F)(F)F)C1] |
| | 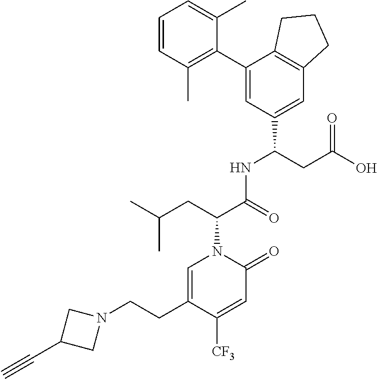 [SMILES: C#CC1CN(CCc2cn([C@H](CC(C)C)C(=O)N[C@@H](CC(=O)O)c3cc4c(c(-c5c(C)cccc5C)c3)CCC4)c(=O)cc2C(F)(F)F)C1] |
| | 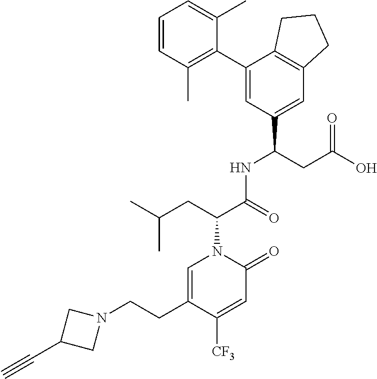 [SMILES: C#CC1CN(CCc2cn([C@H](CC(C)C)C(=O)N[C@H](CC(=O)O)c3cc4c(c(-c5c(C)cccc5C)c3)CCC4)c(=O)cc2C(F)(F)F)C1] |
| 81 | 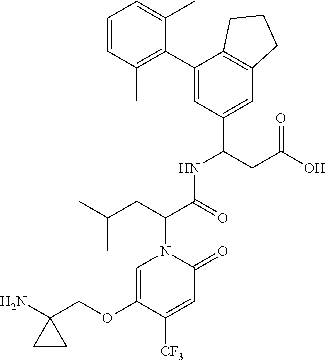 [SMILES: Cc1cccc(C)c1-c1cc(C(CC(=O)O)NC(=O)C(CC(C)C)n2cc(OCC3(N)CC3)c(C(F)(F)F)cc2=O)cc2c1CCC2] |
| | 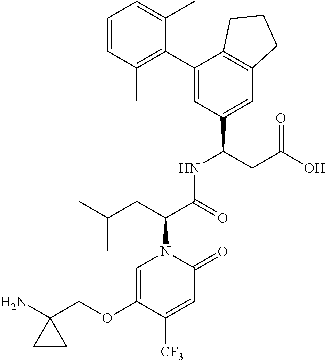 [SMILES: Cc1cccc(C)c1-c1cc([C@@H](CC(=O)O)NC(=O)[C@H](CC(C)C)n2cc(OCC3(N)CC3)c(C(F)(F)F)cc2=O)cc2c1CCC2] |
| | 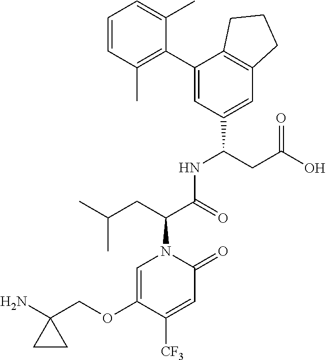 [SMILES: Cc1cccc(C)c1-c1cc([C@H](CC(=O)O)NC(=O)[C@H](CC(C)C)n2cc(OCC3(N)CC3)c(C(F)(F)F)cc2=O)cc2c1CCC2] |
| | 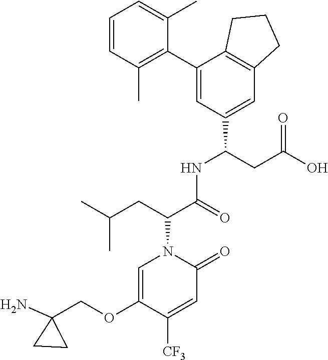 |
| | 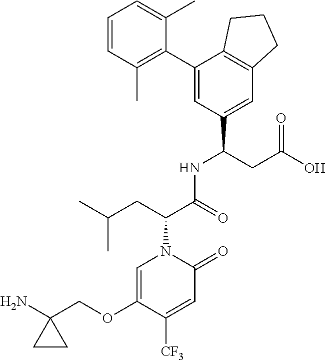 [SMILES: Cc1cccc(C)c1-c1cc([C@@H](CC(=O)O)NC(=O)[C@@H](CC(C)C)n2cc(OCC3(N)CC3)c(C(F)(F)F)cc2=O)cc2c1CCC2] |
| 82 | 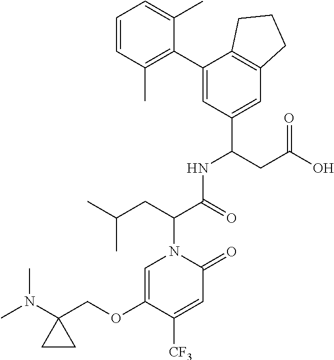 [SMILES: Cc1cccc(C)c1-c1cc(C(CC(=O)O)NC(=O)C(CC(C)C)n2cc(OCC3(N(C)C)CC3)c(C(F)(F)F)cc2=O)cc2c1CCC2] |
| | 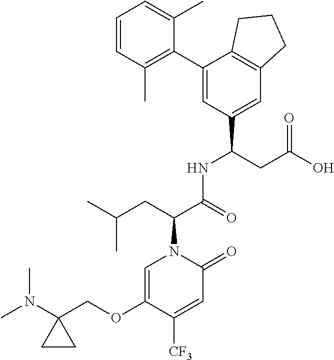 [SMILES: Cc1cccc(C)c1-c1cc([C@@H](CC(=O)O)NC(=O)[C@H](CC(C)C)n2cc(OCC3(N(C)C)CC3)c(C(F)(F)F)cc2=O)cc2c1CCC2] |
| | 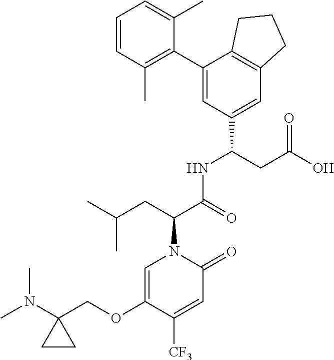 [SMILES: Cc1cccc(C)c1-c1cc([C@H](CC(=O)O)NC(=O)[C@H](CC(C)C)n2cc(OCC3(N(C)C)CC3)c(C(F)(F)F)cc2=O)cc2c1CCC2] |
| | 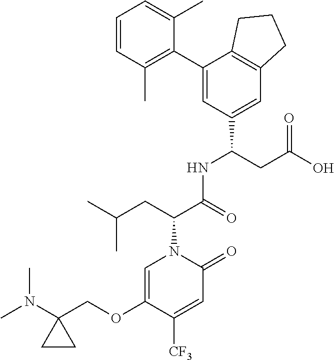 [SMILES: Cc1cccc(C)c1-c1cc([C@H](CC(=O)O)NC(=O)[C@@H](CC(C)C)n2cc(OCC3(N(C)C)CC3)c(C(F)(F)F)cc2=O)cc2c1CCC2] |
| | 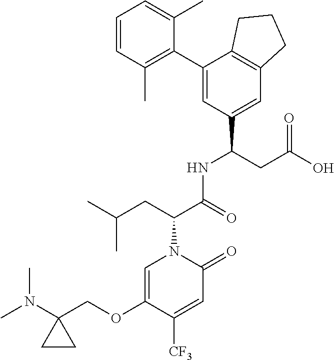 [SMILES: Cc1cccc(C)c1-c1cc([C@@H](CC(=O)O)NC(=O)[C@@H](CC(C)C)n2cc(OCC3(N(C)C)CC3)c(C(F)(F)F)cc2=O)cc2c1CCC2] |
| 83 | 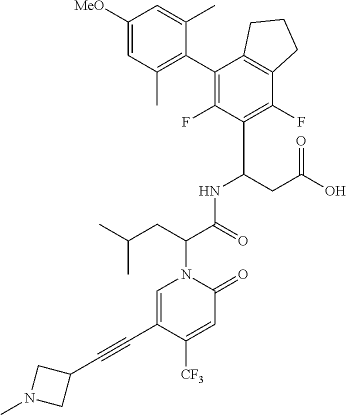 [SMILES: COc1cc(C)c(-c2c(F)c(C(CC(=O)O)NC(=O)C(CC(C)C)n3cc(C#CC4CN(C)C4)c(C(F)(F)F)cc3=O)c(F)c3c2CCC3)c(C)c1] |
| | 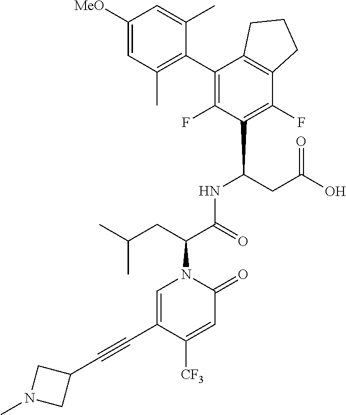 [SMILES: COc1cc(C)c(-c2c(F)c([C@@H](CC(=O)O)NC(=O)[C@H](CC(C)C)n3cc(C#CC4CN(C)C4)c(C(F)(F)F)cc3=O)c(F)c3c2CCC3)c(C)c1] |
| | 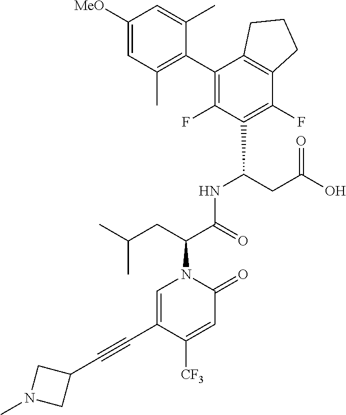 [SMILES: COc1cc(C)c(-c2c(F)c([C@H](CC(=O)O)NC(=O)[C@H](CC(C)C)n3cc(C#CC4CN(C)C4)c(C(F)(F)F)cc3=O)c(F)c3c2CCC3)c(C)c1] |
| | 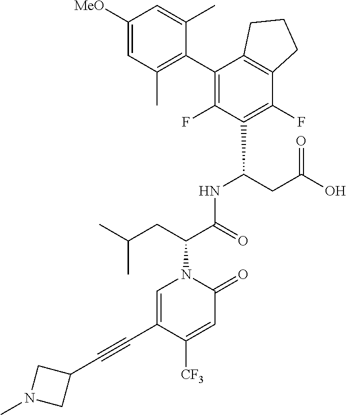 [SMILES: COc1cc(C)c(-c2c(F)c([C@H](CC(=O)O)NC(=O)[C@@H](CC(C)C)n3cc(C#CC4CN(C)C4)c(C(F)(F)F)cc3=O)c(F)c3c2CCC3)c(C)c1] |
| | 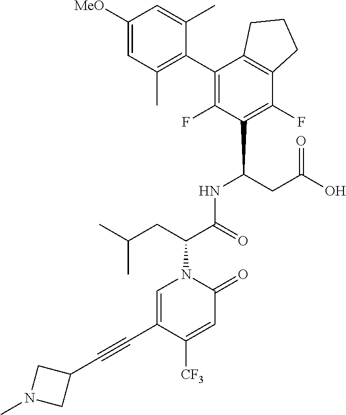 [SMILES: COc1cc(C)c(-c2c(F)c([C@@H](CC(=O)O)NC(=O)[C@@H](CC(C)C)n3cc(C#CC4CN(C)C4)c(C(F)(F)F)cc3=O)c(F)c3c2CCC3)c(C)c1] |
| 84 | 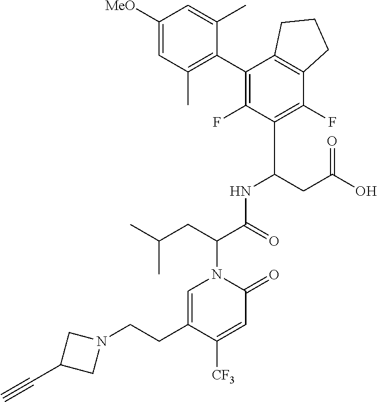 [SMILES: C#CC1CN(CCc2cn(C(CC(C)C)C(=O)NC(CC(=O)O)c3c(F)c4c(c(-c5c(C)cc(OC)cc5C)c3F)CCC4)c(=O)cc2C(F)(F)F)C1] |
| | 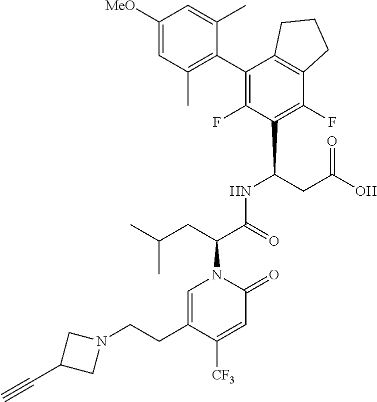 [SMILES: C#CC1CN(CCc2cn([C@@H](CC(C)C)C(=O)N[C@H](CC(=O)O)c3c(F)c4c(c(-c5c(C)cc(OC)cc5C)c3F)CCC4)c(=O)cc2C(F)(F)F)C1] |
| | 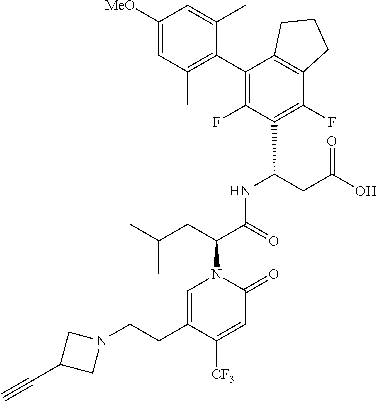 [SMILES: C#CC1CN(CCc2cn([C@@H](CC(C)C)C(=O)N[C@@H](CC(=O)O)c3c(F)c4c(c(-c5c(C)cc(OC)cc5C)c3F)CCC4)c(=O)cc2C(F)(F)F)C1] |
| | 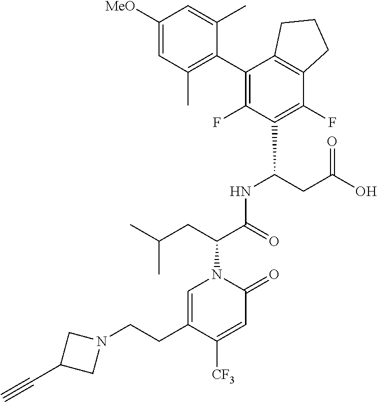 [SMILES: C#CC1CN(CCc2cn([C@H](CC(C)C)C(=O)N[C@@H](CC(=O)O)c3c(F)c4c(c(-c5c(C)cc(OC)cc5C)c3F)CCC4)c(=O)cc2C(F)(F)F)C1] |
| | 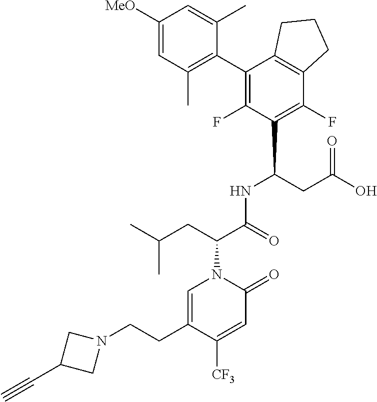 [SMILES: C#CC1CN(CCc2cn([C@H](CC(C)C)C(=O)N[C@H](CC(=O)O)c3c(F)c4c(c(-c5c(C)cc(OC)cc5C)c3F)CCC4)c(=O)cc2C(F)(F)F)C1] |
| 85 | 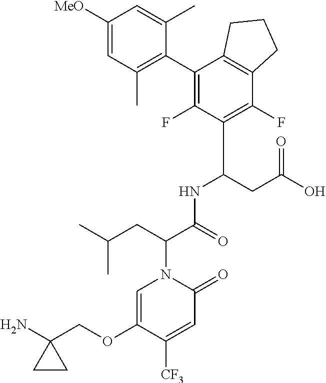 [SMILES: COc1cc(C)c(-c2c(F)c(C(CC(=O)O)NC(=O)C(CC(C)C)n3cc(OCC4(N)CC4)c(C(F)(F)F)cc3=O)c(F)c3c2CCC3)c(C)c1] |
| | 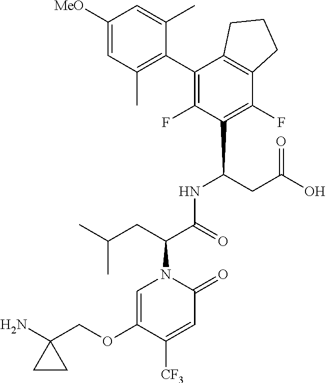 [SMILES: COc1cc(C)c(-c2c(F)c([C@@H](CC(=O)O)NC(=O)[C@H](CC(C)C)n3cc(OCC4(N)CC4)c(C(F)(F)F)cc3=O)c(F)c3c2CCC3)c(C)c1] |
| | 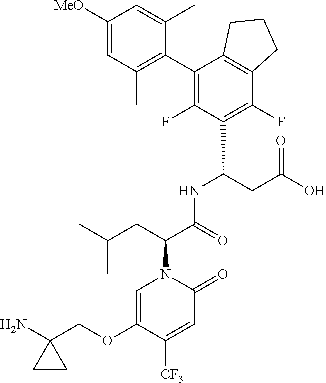 [SMILES: COc1cc(C)c(-c2c(F)c([C@H](CC(=O)O)NC(=O)[C@H](CC(C)C)n3cc(OCC4(N)CC4)c(C(F)(F)F)cc3=O)c(F)c3c2CCC3)c(C)c1] |
| | 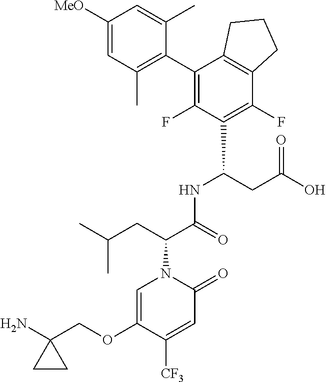 [SMILES: COc1cc(C)c(-c2c(F)c([C@H](CC(=O)O)NC(=O)[C@@H](CC(C)C)n3cc(OCC4(N)CC4)c(C(F)(F)F)cc3=O)c(F)c3c2CCC3)c(C)c1] |
| | 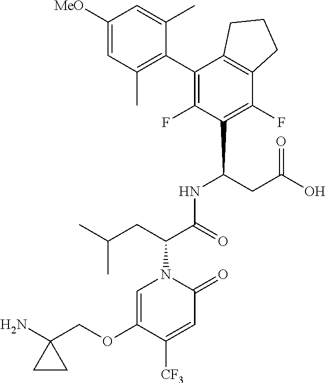 [SMILES: COc1cc(C)c(-c2c(F)c([C@@H](CC(=O)O)NC(=O)[C@@H](CC(C)C)n3cc(OCC4(N)CC4)c(C(F)(F)F)cc3=O)c(F)c3c2CCC3)c(C)c1] |
| 86 | 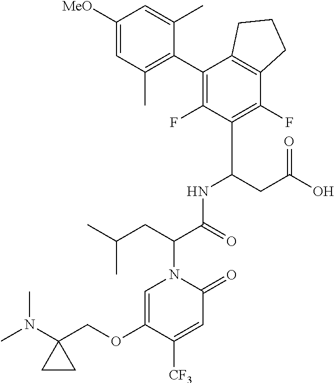 [SMILES: COc1cc(C)c(-c2c(F)c(C(CC(=O)O)NC(=O)C(CC(C)C)n3cc(OCC4(N(C)C)CC4)c(C(F)(F)F)cc3=O)c(F)c3c2CCC3)c(C)c1] |
| | 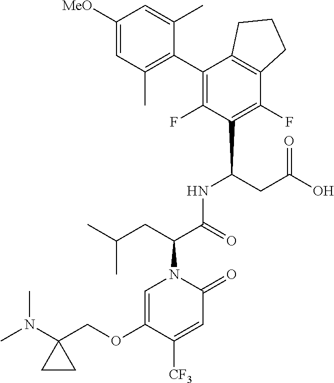 [SMILES: COc1cc(C)c(-c2c(F)c([C@@H](CC(=O)O)NC(=O)[C@H](CC(C)C)n3cc(OCC4(N(C)C)CC4)c(C(F)(F)F)cc3=O)c(F)c3c2CCC3)c(C)c1] |
| | 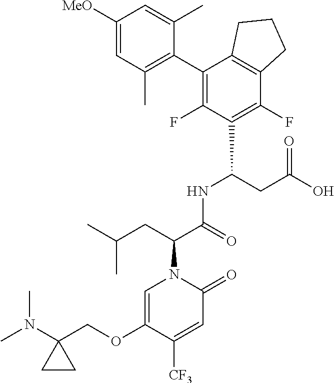 [SMILES: COc1cc(C)c(-c2c(F)c([C@H](CC(=O)O)NC(=O)[C@H](CC(C)C)n3cc(OCC4(N(C)C)CC4)c(C(F)(F)F)cc3=O)c(F)c3c2CCC3)c(C)c1] |
| | 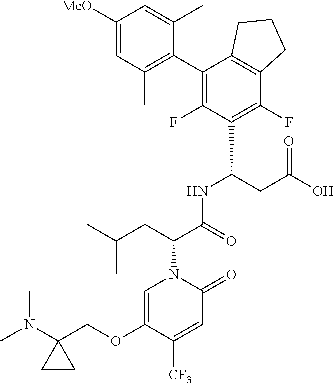 [SMILES: COc1cc(C)c(-c2c(F)c([C@H](CC(=O)O)NC(=O)[C@@H](CC(C)C)n3cc(OCC4(N(C)C)CC4)c(C(F)(F)F)cc3=O)c(F)c3c2CCC3)c(C)c1] |
| | 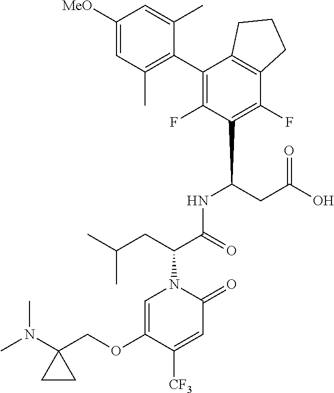 [SMILES: COc1cc(C)c(-c2c(F)c([C@@H](CC(=O)O)NC(=O)[C@@H](CC(C)C)n3cc(OCC4(N(C)C)CC4)c(C(F)(F)F)cc3=O)c(F)c3c2CCC3)c(C)c1] |
| 87 | 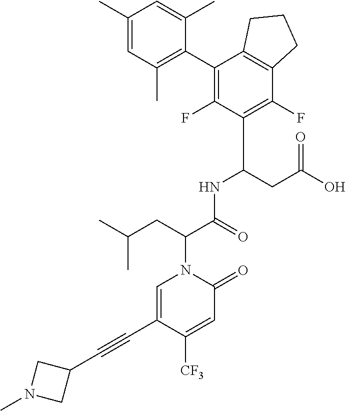 [SMILES: Cc1cc(C)c(-c2c(F)c(C(CC(=O)O)NC(=O)C(CC(C)C)n3cc(C#CC4CN(C)C4)c(C(F)(F)F)cc3=O)c(F)c3c2CCC3)c(C)c1] |
| | 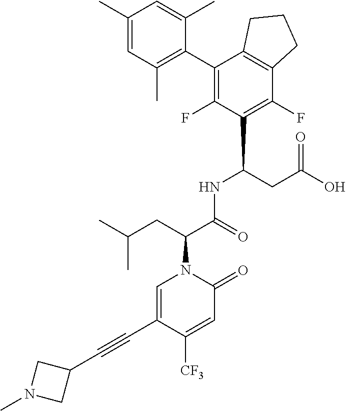 [SMILES: Cc1cc(C)c(-c2c(F)c([C@@H](CC(=O)O)NC(=O)[C@H](CC(C)C)n3cc(C#CC4CN(C)C4)c(C(F)(F)F)cc3=O)c(F)c3c2CCC3)c(C)c1] |
| | 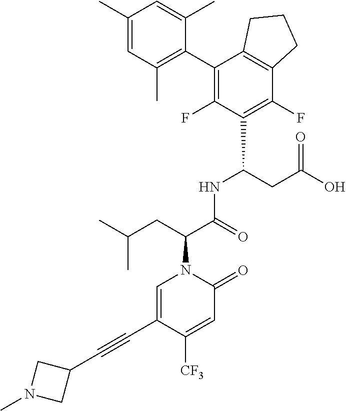 [SMILES: Cc1cc(C)c(-c2c(F)c([C@H](CC(=O)O)NC(=O)[C@H](CC(C)C)n3cc(C#CC4CN(C)C4)c(C(F)(F)F)cc3=O)c(F)c3c2CCC3)c(C)c1] |
| | 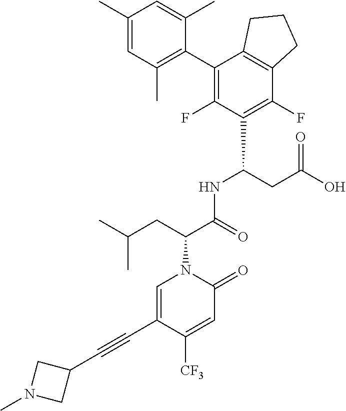 [SMILES: Cc1cc(C)c(-c2c(F)c([C@H](CC(=O)O)NC(=O)[C@@H](CC(C)C)n3cc(C#CC4CN(C)C4)c(C(F)(F)F)cc3=O)c(F)c3c2CCC3)c(C)c1] |
| | 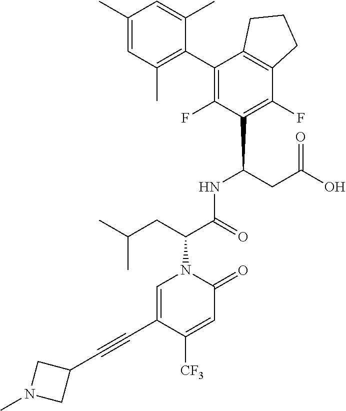 [SMILES: Cc1cc(C)c(-c2c(F)c([C@@H](CC(=O)O)NC(=O)[C@@H](CC(C)C)n3cc(C#CC4CN(C)C4)c(C(F)(F)F)cc3=O)c(F)c3c2CCC3)c(C)c1] |
| 88 | 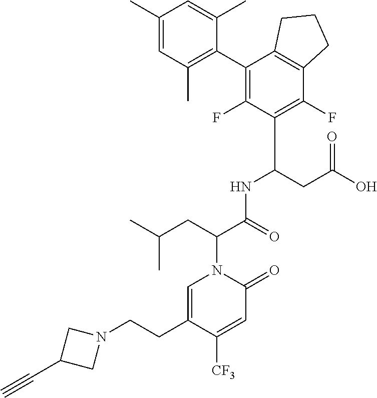 [SMILES: C#CC1CN(CCc2cn(C(CC(C)C)C(=O)NC(CC(=O)O)c3c(F)c4c(c(-c5c(C)cc(C)cc5C)c3F)CCC4)c(=O)cc2C(F)(F)F)C1] |
| | 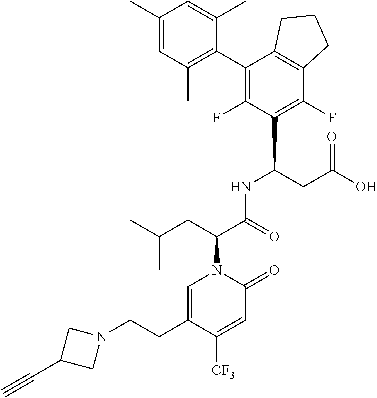 [SMILES: C#CC1CN(CCc2cn([C@@H](CC(C)C)C(=O)N[C@H](CC(=O)O)c3c(F)c4c(c(-c5c(C)cc(C)cc5C)c3F)CCC4)c(=O)cc2C(F)(F)F)C1] |
| | 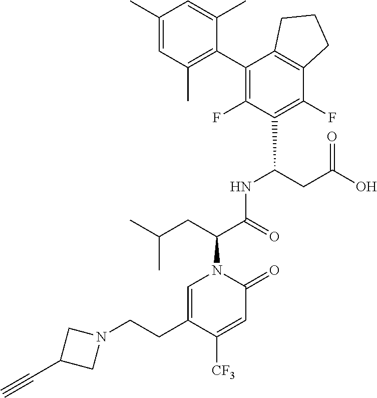 [SMILES: C#CC1CN(CCc2cn([C@@H](CC(C)C)C(=O)N[C@@H](CC(=O)O)c3c(F)c4c(c(-c5c(C)cc(C)cc5C)c3F)CCC4)c(=O)cc2C(F)(F)F)C1] |
| | 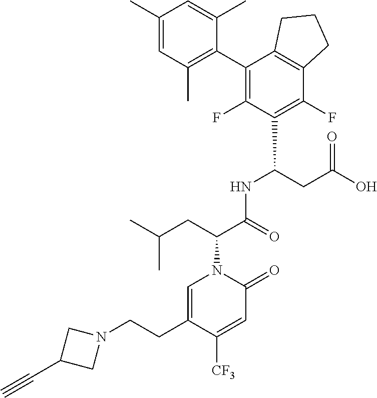 [SMILES: C#CC1CN(CCc2cn([C@H](CC(C)C)C(=O)N[C@@H](CC(=O)O)c3c(F)c4c(c(-c5c(C)cc(C)cc5C)c3F)CCC4)c(=O)cc2C(F)(F)F)C1] |
| | 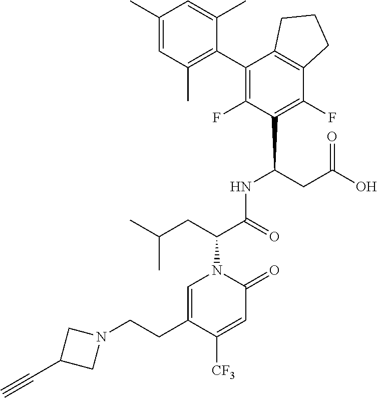 [SMILES: C#CC1CN(CCc2cn([C@H](CC(C)C)C(=O)N[C@H](CC(=O)O)c3c(F)c4c(c(-c5c(C)cc(C)cc5C)c3F)CCC4)c(=O)cc2C(F)(F)F)C1] |
| 89 | 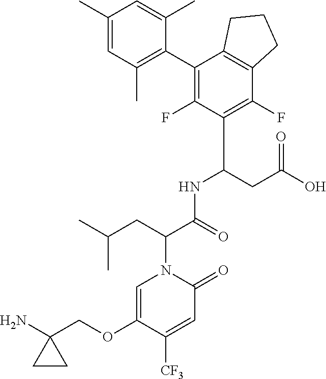 [SMILES: Cc1cc(C)c(-c2c(F)c(C(CC(=O)O)NC(=O)C(CC(C)C)n3cc(OCC4(N)CC4)c(C(F)(F)F)cc3=O)c(F)c3c2CCC3)c(C)c1] |
| | 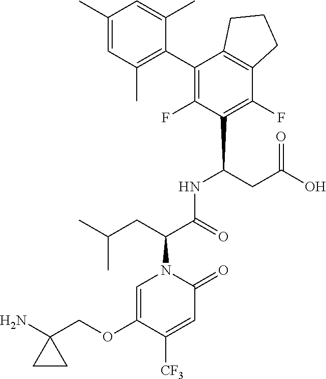 [SMILES: Cc1cc(C)c(-c2c(F)c([C@@H](CC(=O)O)NC(=O)[C@H](CC(C)C)n3cc(OCC4(N)CC4)c(C(F)(F)F)cc3=O)c(F)c3c2CCC3)c(C)c1] |
| | 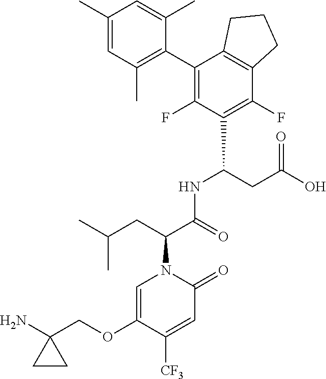 [SMILES: Cc1cc(C)c(-c2c(F)c([C@H](CC(=O)O)NC(=O)[C@H](CC(C)C)n3cc(OCC4(N)CC4)c(C(F)(F)F)cc3=O)c(F)c3c2CCC3)c(C)c1] |
| | 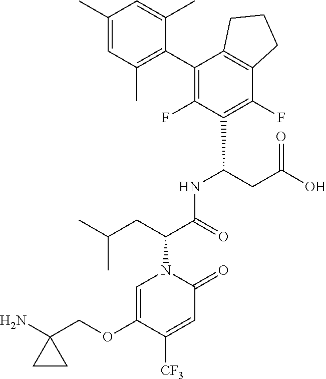 [SMILES: Cc1cc(C)c(-c2c(F)c([C@H](CC(=O)O)NC(=O)[C@@H](CC(C)C)n3cc(OCC4(N)CC4)c(C(F)(F)F)cc3=O)c(F)c3c2CCC3)c(C)c1] |
| | 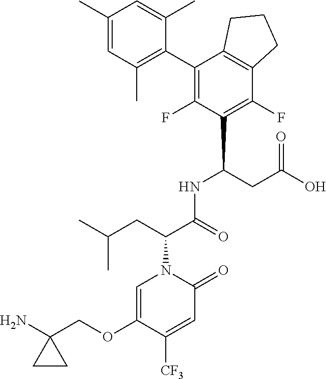 [SMILES: Cc1cc(C)c(-c2c(F)c([C@@H](CC(=O)O)NC(=O)[C@@H](CC(C)C)n3cc(OCC4(N)CC4)c(C(F)(F)F)cc3=O)c(F)c3c2CCC3)c(C)c1] |
| 90 | 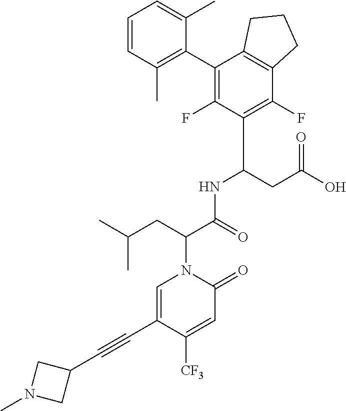 [SMILES: Cc1cccc(C)c1-c1c(F)c(C(CC(=O)O)NC(=O)C(CC(C)C)n2cc(C#CC3CN(C)C3)c(C(F)(F)F)cc2=O)c(F)c2c1CCC2] |
| | 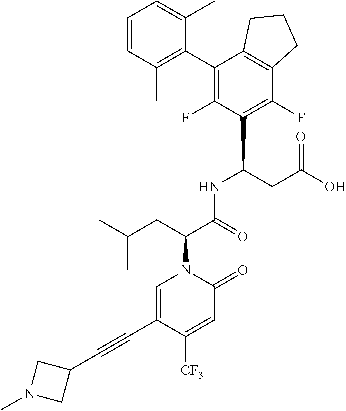 [SMILES: Cc1cccc(C)c1-c1c(F)c([C@@H](CC(=O)O)NC(=O)[C@H](CC(C)C)n2cc(C#CC3CN(C)C3)c(C(F)(F)F)cc2=O)c(F)c2c1CCC2] |
| | 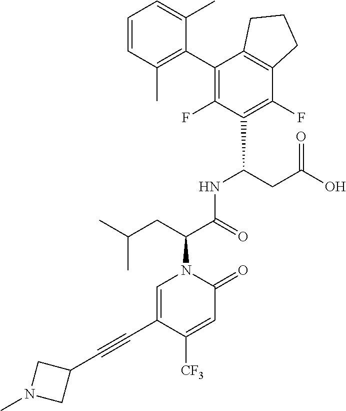 [SMILES: Cc1cccc(C)c1-c1c(F)c([C@H](CC(=O)O)NC(=O)[C@H](CC(C)C)n2cc(C#CC3CN(C)C3)c(C(F)(F)F)cc2=O)c(F)c2c1CCC2] |
| | 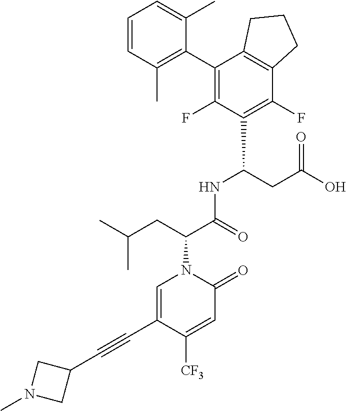 [SMILES: Cc1cccc(C)c1-c1c(F)c([C@H](CC(=O)O)NC(=O)[C@@H](CC(C)C)n2cc(C#CC3CN(C)C3)c(C(F)(F)F)cc2=O)c(F)c2c1CCC2] |
| | 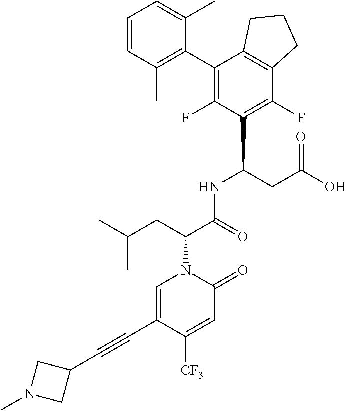 [SMILES: Cc1cccc(C)c1-c1c(F)c([C@@H](CC(=O)O)NC(=O)[C@@H](CC(C)C)n2cc(C#CC3CN(C)C3)c(C(F)(F)F)cc2=O)c(F)c2c1CCC2] |
| 91 | 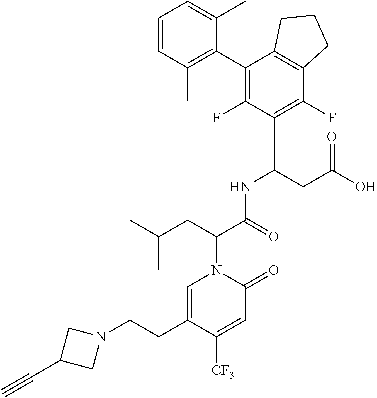 [SMILES: C#CC1CN(CCc2cn(C(CC(C)C)C(=O)NC(CC(=O)O)c3c(F)c4c(c(-c5c(C)cccc5C)c3F)CCC4)c(=O)cc2C(F)(F)F)C1] |
| | 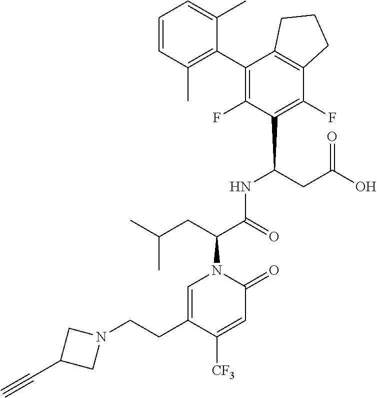 [SMILES: C#CC1CN(CCc2cn([C@@H](CC(C)C)C(=O)N[C@H](CC(=O)O)c3c(F)c4c(c(-c5c(C)cccc5C)c3F)CCC4)c(=O)cc2C(F)(F)F)C1] |
| | 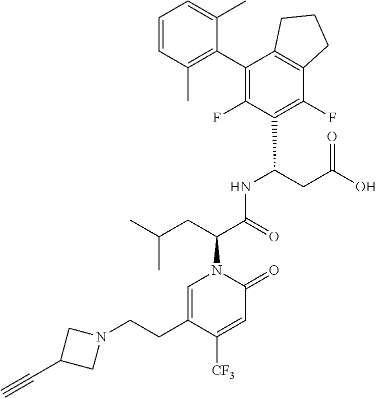 [SMILES: C#CC1CN(CCc2cn([C@@H](CC(C)C)C(=O)N[C@@H](CC(=O)O)c3c(F)c4c(c(-c5c(C)cccc5C)c3F)CCC4)c(=O)cc2C(F)(F)F)C1] |
| | 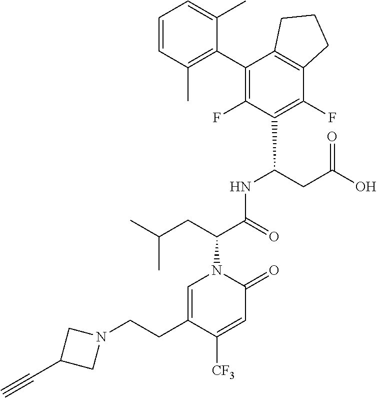 [SMILES: C#CC1CN(CCc2cn([C@H](CC(C)C)C(=O)N[C@@H](CC(=O)O)c3c(F)c4c(c(-c5c(C)cccc5C)c3F)CCC4)c(=O)cc2C(F)(F)F)C1] |
| | 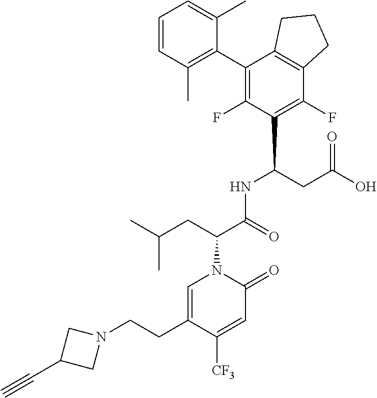 [SMILES: C#CC1CN(CCc2cn([C@H](CC(C)C)C(=O)N[C@H](CC(=O)O)c3c(F)c4c(c(-c5c(C)cccc5C)c3F)CCC4)c(=O)cc2C(F)(F)F)C1] |
| 92 | 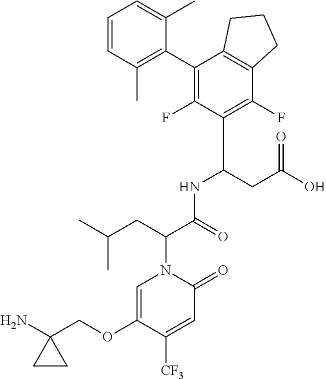 [SMILES: Cc1cccc(C)c1-c1c(F)c(C(CC(=O)O)NC(=O)C(CC(C)C)n2cc(OCC3(N)CC3)c(C(F)(F)F)cc2=O)c(F)c2c1CCC2] |
| | 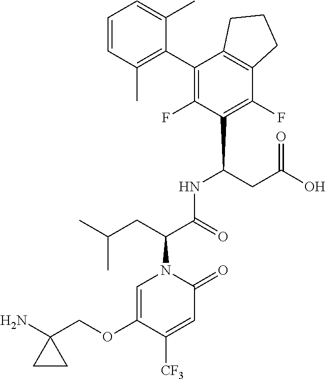 [SMILES: Cc1cccc(C)c1-c1c(F)c([C@@H](CC(=O)O)NC(=O)[C@H](CC(C)C)n2cc(OCC3(N)CC3)c(C(F)(F)F)cc2=O)c(F)c2c1CCC2] |
| | 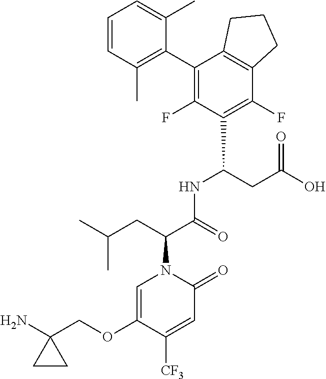 [SMILES: Cc1cccc(C)c1-c1c(F)c([C@H](CC(=O)O)NC(=O)[C@H](CC(C)C)n2cc(OCC3(N)CC3)c(C(F)(F)F)cc2=O)c(F)c2c1CCC2] |
| | 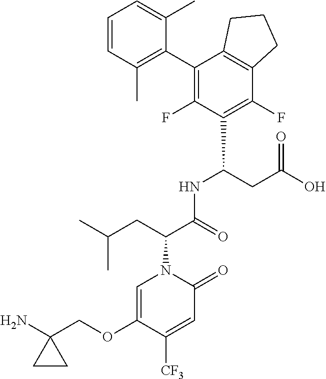 [SMILES: Cc1cccc(C)c1-c1c(F)c([C@H](CC(=O)O)NC(=O)[C@@H](CC(C)C)n2cc(OCC3(N)CC3)c(C(F)(F)F)cc2=O)c(F)c2c1CCC2] |
| | 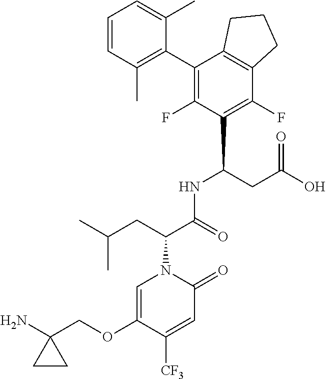 [SMILES: Cc1cccc(C)c1-c1c(F)c([C@@H](CC(=O)O)NC(=O)[C@@H](CC(C)C)n2cc(OCC3(N)CC3)c(C(F)(F)F)cc2=O)c(F)c2c1CCC2] |
| 93 | 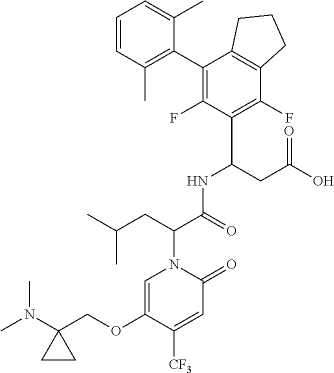 [SMILES: Cc1cccc(C)c1-c1c(F)c(C(CC(=O)O)NC(=O)C(CC(C)C)n2cc(OCC3(N(C)C)CC3)c(C(F)(F)F)cc2=O)c(F)c2c1CCC2] |
| | 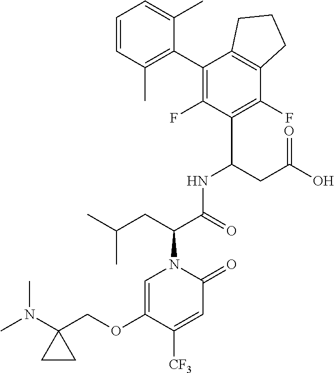 [SMILES: Cc1cccc(C)c1-c1c(F)c(C(CC(=O)O)NC(=O)[C@H](CC(C)C)n2cc(OCC3(N(C)C)CC3)c(C(F)(F)F)cc2=O)c(F)c2c1CCC2] |
| | 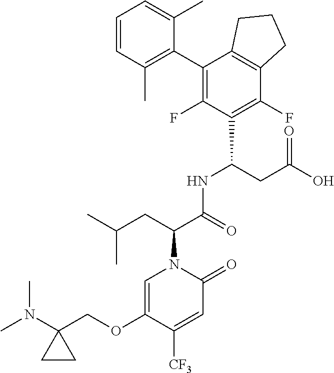 [SMILES: Cc1cccc(C)c1-c1c(F)c([C@H](CC(=O)O)NC(=O)[C@H](CC(C)C)n2cc(OCC3(N(C)C)CC3)c(C(F)(F)F)cc2=O)c(F)c2c1CCC2] |
| | 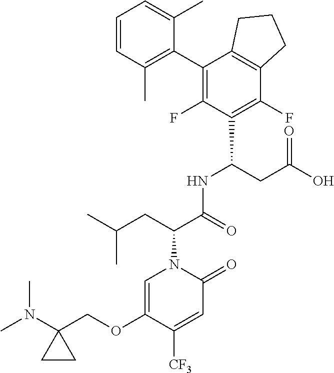 [SMILES: Cc1cccc(C)c1-c1c(F)c([C@H](CC(=O)O)NC(=O)[C@@H](CC(C)C)n2cc(OCC3(N(C)C)CC3)c(C(F)(F)F)cc2=O)c(F)c2c1CCC2] |
| | 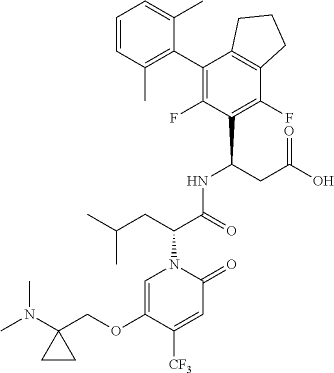 [SMILES: Cc1cccc(C)c1-c1c(F)c([C@@H](CC(=O)O)NC(=O)[C@@H](CC(C)C)n2cc(OCC3(N(C)C)CC3)c(C(F)(F)F)cc2=O)c(F)c2c1CCC2] |
| 94 | 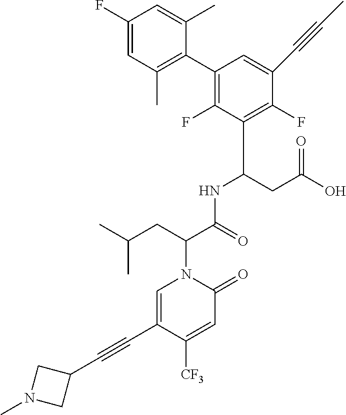 [SMILES: CC#Cc1cc(-c2c(C)cc(F)cc2C)c(F)c(C(CC(=O)O)NC(=O)C(CC(C)C)n2cc(C#CC3CN(C)C3)c(C(F)(F)F)cc2=O)c1F] |
| | 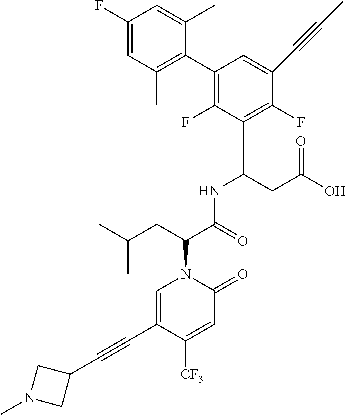 |
| | 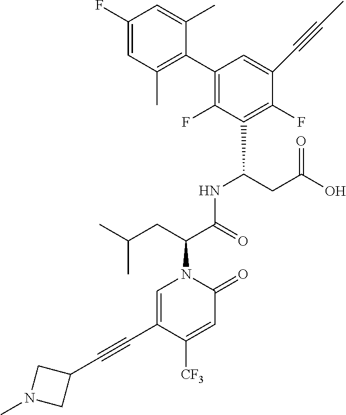 [SMILES: CC#Cc1cc(-c2c(C)cc(F)cc2C)c(F)c([C@H](CC(=O)O)NC(=O)[C@H](CC(C)C)n2cc(C#CC3CN(C)C3)c(C(F)(F)F)cc2=O)c1F] |
| | 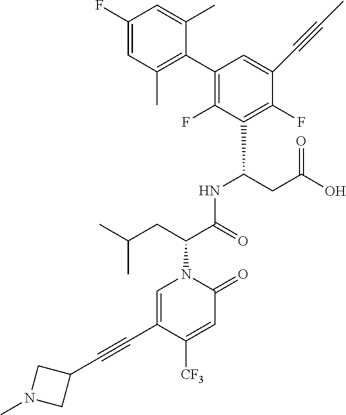 [SMILES: CC#Cc1cc(-c2c(C)cc(F)cc2C)c(F)c([C@H](CC(=O)O)NC(=O)[C@@H](CC(C)C)n2cc(C#CC3CN(C)C3)c(C(F)(F)F)cc2=O)c1F] |
| | 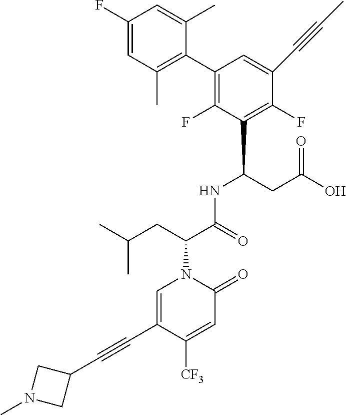 [SMILES: CC#Cc1cc(-c2c(C)cc(F)cc2C)c(F)c([C@@H](CC(=O)O)NC(=O)[C@@H](CC(C)C)n2cc(C#CC3CN(C)C3)c(C(F)(F)F)cc2=O)c1F] |
| 95 | 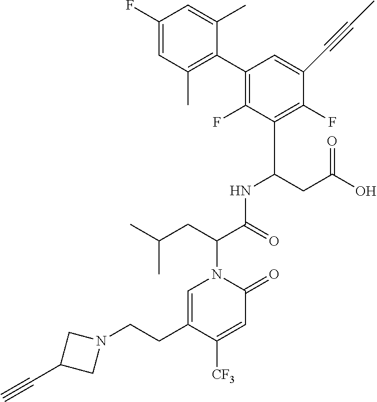 [SMILES: C#CC1CN(CCc2cn(C(CC(C)C)C(=O)NC(CC(=O)O)c3c(F)c(C#CC)cc(-c4c(C)cc(F)cc4C)c3F)c(=O)cc2C(F)(F)F)C1] |
| | 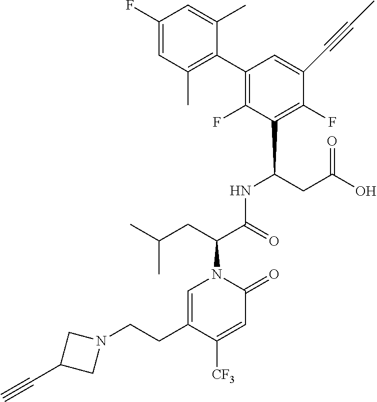 [SMILES: C#CC1CN(CCc2cn([C@@H](CC(C)C)C(=O)N[C@H](CC(=O)O)c3c(F)c(C#CC)cc(-c4c(C)cc(F)cc4C)c3F)c(=O)cc2C(F)(F)F)C1] |
| | 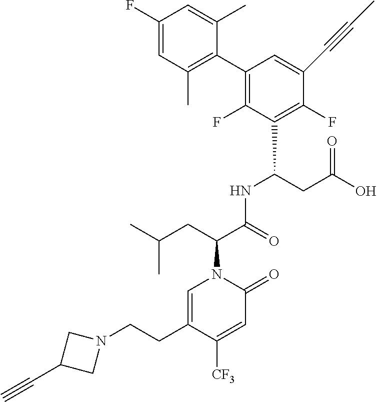 [SMILES: C#CC1CN(CCc2cn([C@@H](CC(C)C)C(=O)N[C@@H](CC(=O)O)c3c(F)c(C#CC)cc(-c4c(C)cc(F)cc4C)c3F)c(=O)cc2C(F)(F)F)C1] |
| | 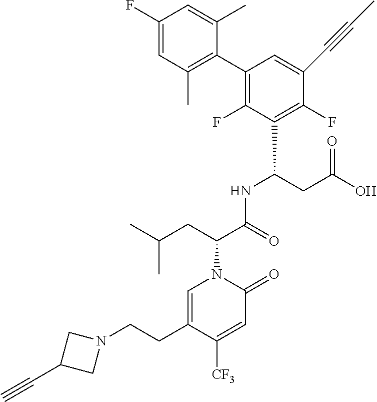 [SMILES: C#CC1CN(CCc2cn([C@H](CC(C)C)C(=O)N[C@@H](CC(=O)O)c3c(F)c(C#CC)cc(-c4c(C)cc(F)cc4C)c3F)c(=O)cc2C(F)(F)F)C1] |
| | 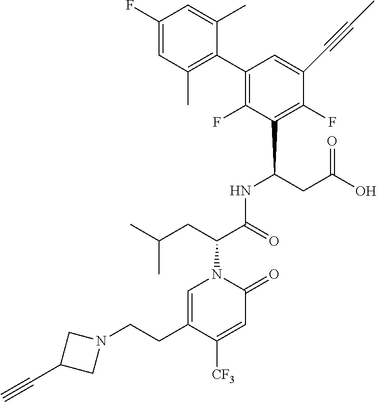 [SMILES: C#CC1CN(CCc2cn([C@H](CC(C)C)C(=O)N[C@H](CC(=O)O)c3c(F)c(C#CC)cc(-c4c(C)cc(F)cc4C)c3F)c(=O)cc2C(F)(F)F)C1] |
| 96 | 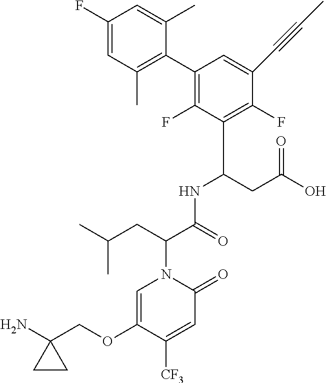 [SMILES: CC#Cc1cc(-c2c(C)cc(F)cc2C)c(F)c(C(CC(=O)O)NC(=O)C(CC(C)C)n2cc(OCC3(N)CC3)c(C(F)(F)F)cc2=O)c1F] |
| | 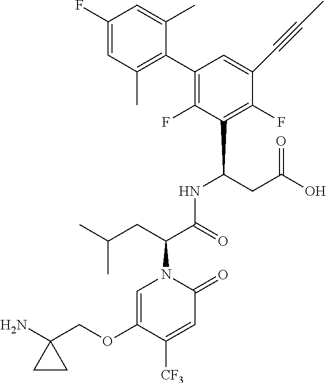 [SMILES: CC#Cc1cc(-c2c(C)cc(F)cc2C)c(F)c([C@@H](CC(=O)O)NC(=O)[C@H](CC(C)C)n2cc(OCC3(N)CC3)c(C(F)(F)F)cc2=O)c1F] |
| | 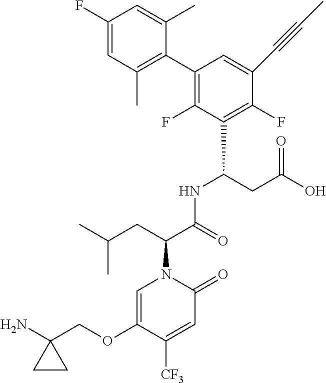 [SMILES: CC#Cc1cc(-c2c(C)cc(F)cc2C)c(F)c([C@H](CC(=O)O)NC(=O)[C@H](CC(C)C)n2cc(OCC3(N)CC3)c(C(F)(F)F)cc2=O)c1F] |
| | 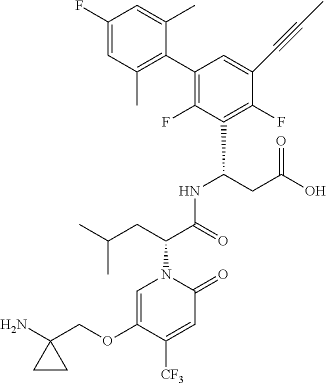 [SMILES: CC#Cc1cc(-c2c(C)cc(F)cc2C)c(F)c([C@H](CC(=O)O)NC(=O)[C@@H](CC(C)C)n2cc(OCC3(N)CC3)c(C(F)(F)F)cc2=O)c1F] |
| | 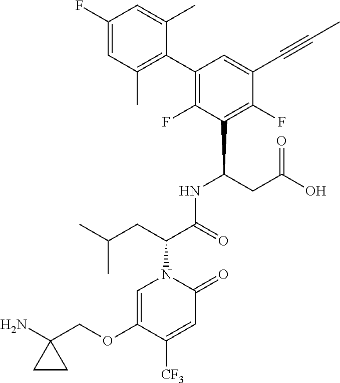 [SMILES: CC#Cc1cc(-c2c(C)cc(F)cc2C)c(F)c([C@@H](CC(=O)O)NC(=O)[C@@H](CC(C)C)n2cc(OCC3(N)CC3)c(C(F)(F)F)cc2=O)c1F] |
| 97 | 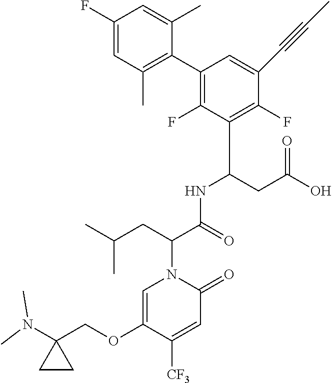 [SMILES: CC#Cc1cc(-c2c(C)cc(F)cc2C)c(F)c(C(CC(=O)O)NC(=O)C(CC(C)C)n2cc(OCC3(N(C)C)CC3)c(C(F)(F)F)cc2=O)c1F] |
| | 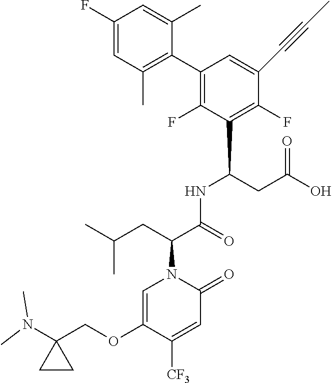 [SMILES: CC#Cc1cc(-c2c(C)cc(F)cc2C)c(F)c([C@@H](CC(=O)O)NC(=O)[C@H](CC(C)C)n2cc(OCC3(N(C)C)CC3)c(C(F)(F)F)cc2=O)c1F] |
| | 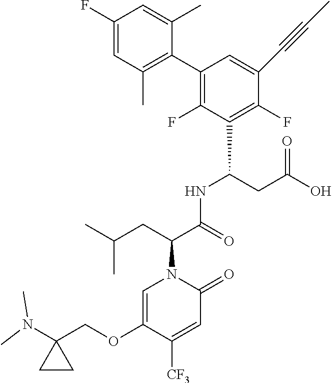 [SMILES: CC#Cc1cc(-c2c(C)cc(F)cc2C)c(F)c([C@H](CC(=O)O)NC(=O)[C@H](CC(C)C)n2cc(OCC3(N(C)C)CC3)c(C(F)(F)F)cc2=O)c1F] |
| | 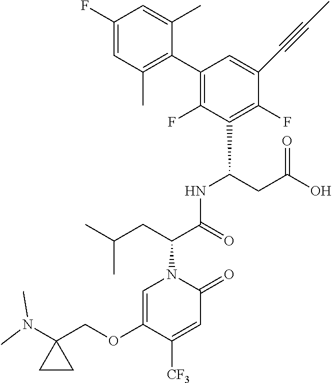 [SMILES: CC#Cc1cc(-c2c(C)cc(F)cc2C)c(F)c([C@H](CC(=O)O)NC(=O)[C@@H](CC(C)C)n2cc(OCC3(N(C)C)CC3)c(C(F)(F)F)cc2=O)c1F] |
| | 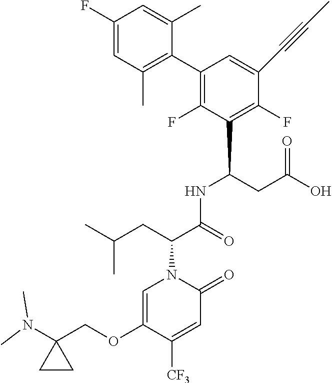 [SMILES: CC#Cc1cc(-c2c(C)cc(F)cc2C)c(F)c([C@@H](CC(=O)O)NC(=O)[C@@H](CC(C)C)n2cc(OCC3(N(C)C)CC3)c(C(F)(F)F)cc2=O)c1F] |
| 98 | 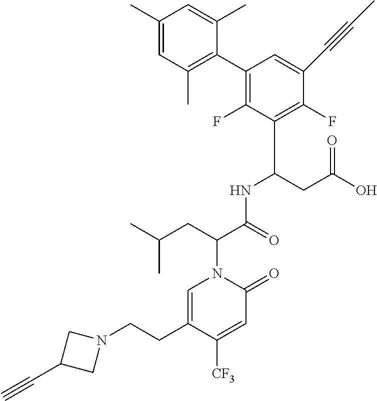 [SMILES: C#CC1CN(CCc2cn(C(CC(C)C)C(=O)NC(CC(=O)O)c3c(F)c(C#CC)cc(-c4c(C)cc(C)cc4C)c3F)c(=O)cc2C(F)(F)F)C1] |
| | 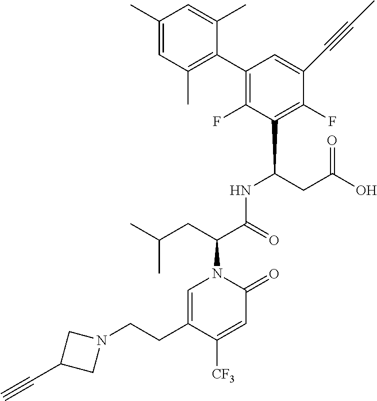 [SMILES: C#CC1CN(CCc2cn([C@@H](CC(C)C)C(=O)N[C@H](CC(=O)O)c3c(F)c(C#CC)cc(-c4c(C)cc(C)cc4C)c3F)c(=O)cc2C(F)(F)F)C1] |
| | 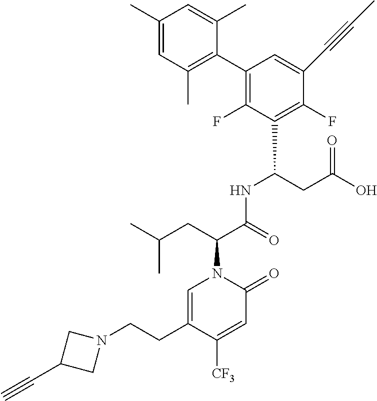 [SMILES: C#CC1CN(CCc2cn([C@@H](CC(C)C)C(=O)N[C@@H](CC(=O)O)c3c(F)c(C#CC)cc(-c4c(C)cc(C)cc4C)c3F)c(=O)cc2C(F)(F)F)C1] |
| | 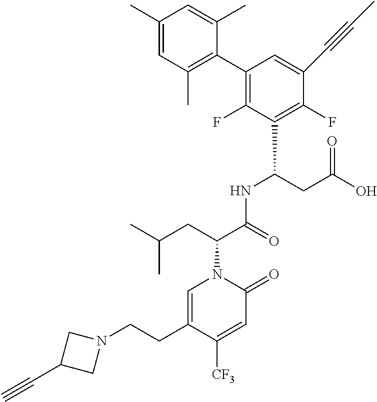 [SMILES: C#CC1CN(CCc2cn([C@H](CC(C)C)C(=O)N[C@@H](CC(=O)O)c3c(F)c(C#CC)cc(-c4c(C)cc(C)cc4C)c3F)c(=O)cc2C(F)(F)F)C1] |
| | 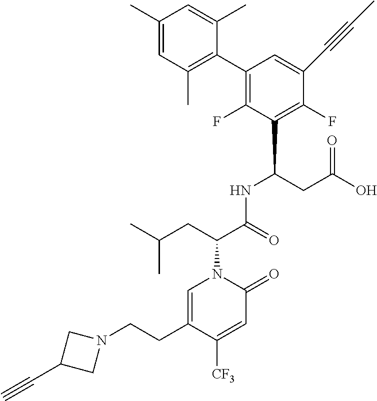 [SMILES: C#CC1CN(CCc2cn([C@H](CC(C)C)C(=O)N[C@H](CC(=O)O)c3c(F)c(C#CC)cc(-c4c(C)cc(C)cc4C)c3F)c(=O)cc2C(F)(F)F)C1] |
| 99 | 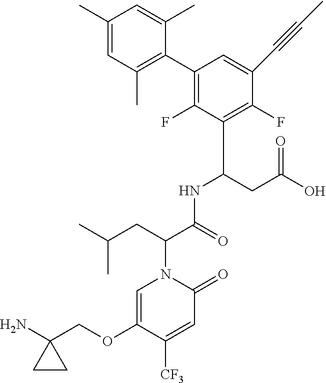 [SMILES: CC#Cc1cc(-c2c(C)cc(C)cc2C)c(F)c(C(CC(=O)O)NC(=O)C(CC(C)C)n2cc(OCC3(N)CC3)c(C(F)(F)F)cc2=O)c1F] |
| | 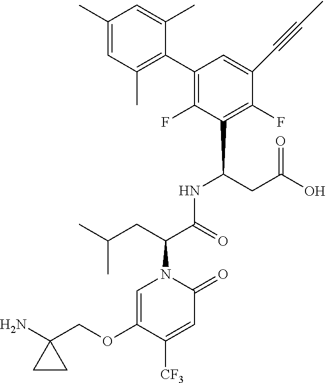 [SMILES: CC#Cc1cc(-c2c(C)cc(C)cc2C)c(F)c([C@@H](CC(=O)O)NC(=O)[C@H](CC(C)C)n2cc(OCC3(N)CC3)c(C(F)(F)F)cc2=O)c1F] |
| | 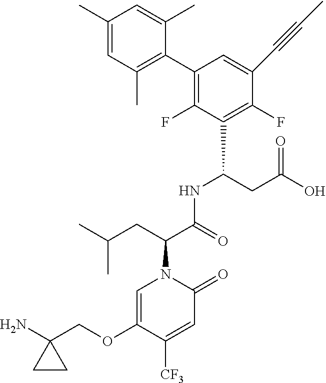 [SMILES: CC#Cc1cc(-c2c(C)cc(C)cc2C)c(F)c([C@H](CC(=O)O)NC(=O)[C@H](CC(C)C)n2cc(OCC3(N)CC3)c(C(F)(F)F)cc2=O)c1F] |
| | 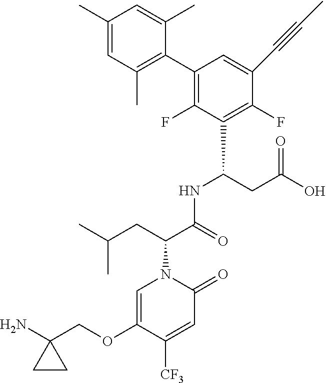 [SMILES: CC#Cc1cc(-c2c(C)cc(C)cc2C)c(F)c([C@H](CC(=O)O)NC(=O)[C@@H](CC(C)C)n2cc(OCC3(N)CC3)c(C(F)(F)F)cc2=O)c1F] |
| | 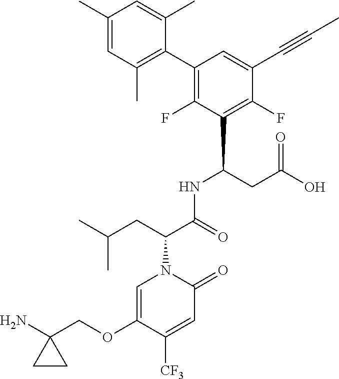 [SMILES: CC#Cc1cc(-c2c(C)cc(C)cc2C)c(F)c([C@@H](CC(=O)O)NC(=O)[C@@H](CC(C)C)n2cc(OCC3(N)CC3)c(C(F)(F)F)cc2=O)c1F] |
| 100 | 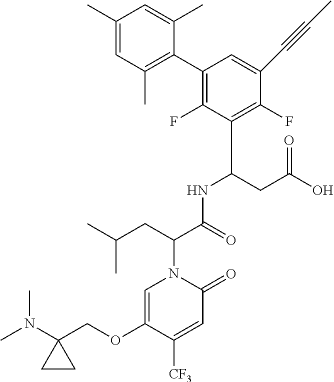 [SMILES: CC#Cc1cc(-c2c(C)cc(C)cc2C)c(F)c(C(CC(=O)O)NC(=O)C(CC(C)C)n2cc(OCC3(N(C)C)CC3)c(C(F)(F)F)cc2=O)c1F] |
| | 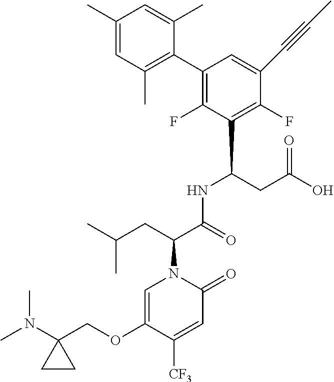 [SMILES: CC#Cc1cc(-c2c(C)cc(C)cc2C)c(F)c([C@@H](CC(=O)O)NC(=O)[C@H](CC(C)C)n2cc(OCC3(N(C)C)CC3)c(C(F)(F)F)cc2=O)c1F] |
| | 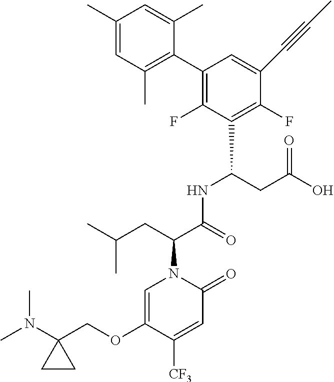 [SMILES: CC#Cc1cc(-c2c(C)cc(C)cc2C)c(F)c([C@H](CC(=O)O)NC(=O)[C@H](CC(C)C)n2cc(OCC3(N(C)C)CC3)c(C(F)(F)F)cc2=O)c1F] |
| | 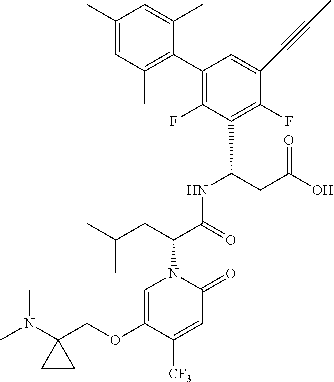 [SMILES: CC#Cc1cc(-c2c(C)cc(C)cc2C)c(F)c([C@H](CC(=O)O)NC(=O)[C@@H](CC(C)C)n2cc(OCC3(N(C)C)CC3)c(C(F)(F)F)cc2=O)c1F] |
| | 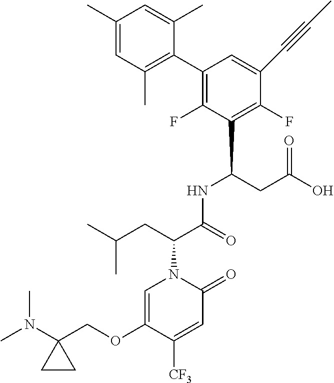 [SMILES: CC#Cc1cc(-c2c(C)cc(C)cc2C)c(F)c([C@@H](CC(=O)O)NC(=O)[C@@H](CC(C)C)n2cc(OCC3(N(C)C)CC3)c(C(F)(F)F)cc2=O)c1F] |
| 101 | 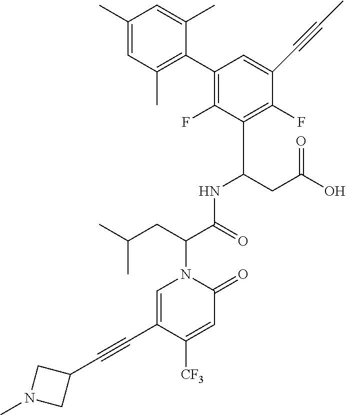 [SMILES: CC#Cc1cc(-c2c(C)cc(C)cc2C)c(F)c(C(CC(=O)O)NC(=O)C(CC(C)C)n2cc(C#CC3CN(C)C3)c(C(F)(F)F)cc2=O)c1F] |
| | 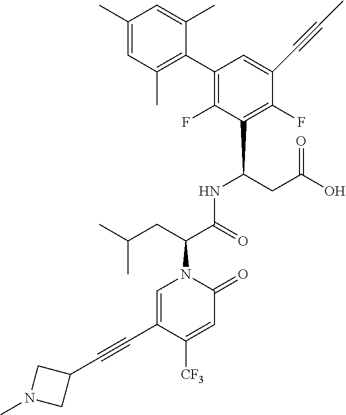 [SMILES: CC#Cc1cc(-c2c(C)cc(C)cc2C)c(F)c([C@@H](CC(=O)O)NC(=O)[C@H](CC(C)C)n2cc(C#CC3CN(C)C3)c(C(F)(F)F)cc2=O)c1F] |
| | 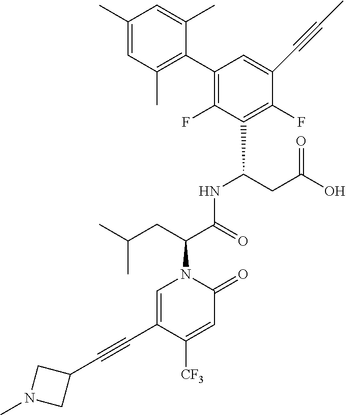 [SMILES: CC#Cc1cc(-c2c(C)cc(C)cc2C)c(F)c([C@H](CC(=O)O)NC(=O)[C@H](CC(C)C)n2cc(C#CC3CN(C)C3)c(C(F)(F)F)cc2=O)c1F] |
| | 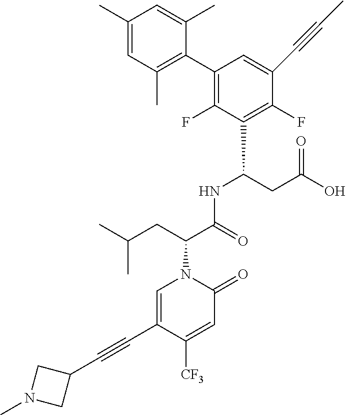 [SMILES: CC#Cc1cc(-c2c(C)cc(C)cc2C)c(F)c([C@H](CC(=O)O)NC(=O)[C@@H](CC(C)C)n2cc(C#CC3CN(C)C3)c(C(F)(F)F)cc2=O)c1F] |
| | 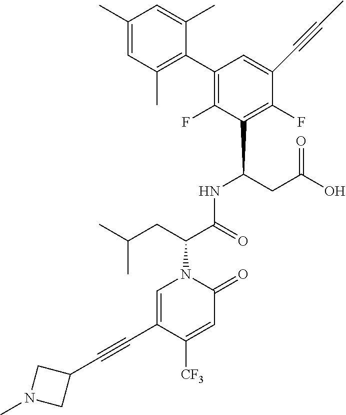 [SMILES: CC#Cc1cc(-c2c(C)cc(C)cc2C)c(F)c([C@@H](CC(=O)O)NC(=O)[C@@H](CC(C)C)n2cc(C#CC3CN(C)C3)c(C(F)(F)F)cc2=O)c1F] |
| 102 | 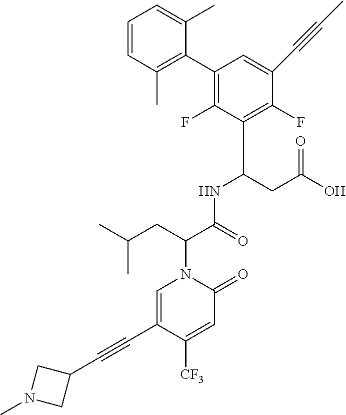 [SMILES: CC#Cc1cc(-c2c(C)cccc2C)c(F)c(C(CC(=O)O)NC(=O)C(CC(C)C)n2cc(C#CC3CN(C)C3)c(C(F)(F)F)cc2=O)c1F] |
| | 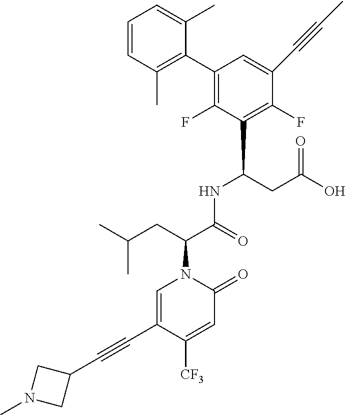 [SMILES: CC#Cc1cc(-c2c(C)cccc2C)c(F)c([C@@H](CC(=O)O)NC(=O)[C@H](CC(C)C)n2cc(C#CC3CN(C)C3)c(C(F)(F)F)cc2=O)c1F] |
| | 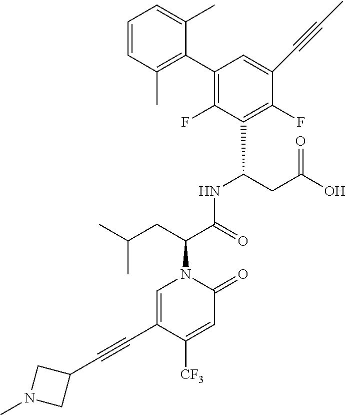 [SMILES: CC#Cc1cc(-c2c(C)cccc2C)c(F)c([C@H](CC(=O)O)NC(=O)[C@H](CC(C)C)n2cc(C#CC3CN(C)C3)c(C(F)(F)F)cc2=O)c1F] |
| | 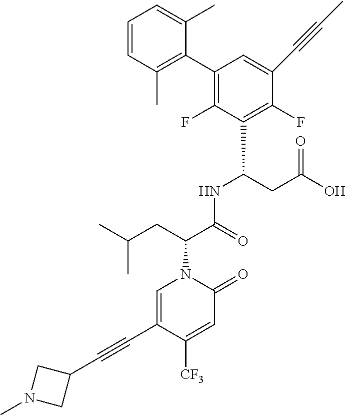 [SMILES: CC#Cc1cc(-c2c(C)cccc2C)c(F)c([C@H](CC(=O)O)NC(=O)[C@@H](CC(C)C)n2cc(C#CC3CN(C)C3)c(C(F)(F)F)cc2=O)c1F] |
| | 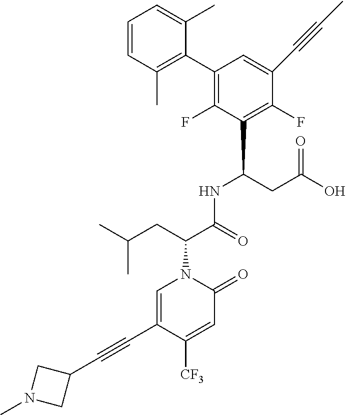 [SMILES: CC#Cc1cc(-c2c(C)cccc2C)c(F)c([C@@H](CC(=O)O)NC(=O)[C@@H](CC(C)C)n2cc(C#CC3CN(C)C3)c(C(F)(F)F)cc2=O)c1F] |
| 103 | 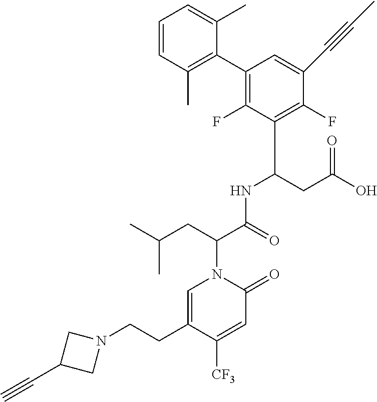 [SMILES: C#CC1CN(CCc2cn(C(CC(C)C)C(=O)NC(CC(=O)O)c3c(F)c(C#CC)cc(-c4c(C)cccc4C)c3F)c(=O)cc2C(F)(F)F)C1] |
| | 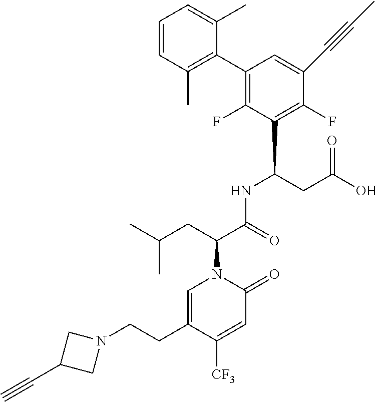 [SMILES: C#CC1CN(CCc2cn([C@@H](CC(C)C)C(=O)N[C@H](CC(=O)O)c3c(F)c(C#CC)cc(-c4c(C)cccc4C)c3F)c(=O)cc2C(F)(F)F)C1] |
| | 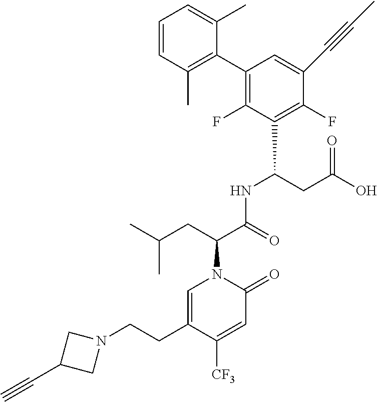 [SMILES: C#CC1CN(CCc2cn([C@@H](CC(C)C)C(=O)N[C@@H](CC(=O)O)c3c(F)c(C#CC)cc(-c4c(C)cccc4C)c3F)c(=O)cc2C(F)(F)F)C1] |
| | 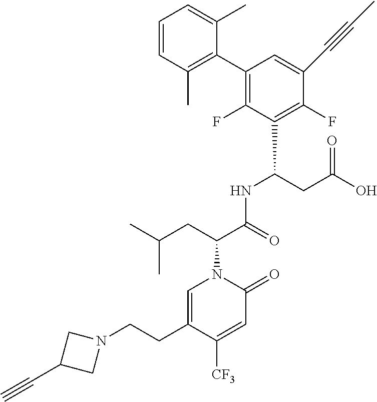 [SMILES: C#CC1CN(CCc2cn([C@H](CC(C)C)C(=O)N[C@@H](CC(=O)O)c3c(F)c(C#CC)cc(-c4c(C)cccc4C)c3F)c(=O)cc2C(F)(F)F)C1] |
| | 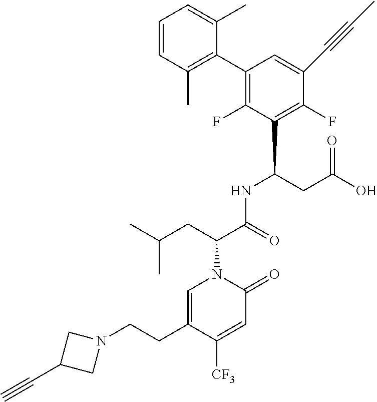 [SMILES: C#CC1CN(CCc2cn([C@H](CC(C)C)C(=O)N[C@H](CC(=O)O)c3c(F)c(C#CC)cc(-c4c(C)cccc4C)c3F)c(=O)cc2C(F)(F)F)C1] |
| 104 | 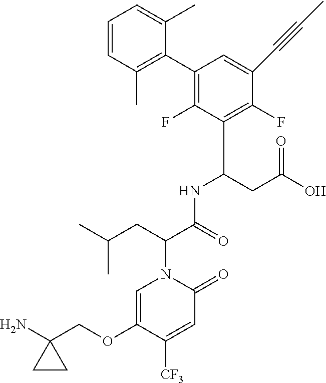 [SMILES: CC#Cc1cc(-c2c(C)cccc2C)c(F)c(C(CC(=O)O)NC(=O)C(CC(C)C)n2cc(OCC3(N)CC3)c(C(F)(F)F)cc2=O)c1F] |
| | 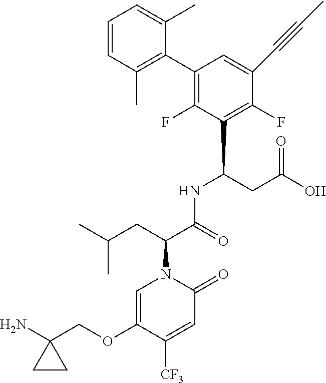 [SMILES: CC#Cc1cc(-c2c(C)cccc2C)c(F)c([C@@H](CC(=O)O)NC(=O)[C@H](CC(C)C)n2cc(OCC3(N)CC3)c(C(F)(F)F)cc2=O)c1F] |
| | 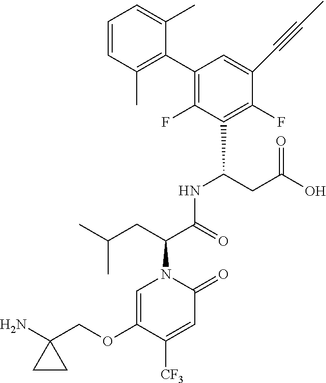 [SMILES: CC#Cc1cc(-c2c(C)cccc2C)c(F)c([C@H](CC(=O)O)NC(=O)[C@H](CC(C)C)n2cc(OCC3(N)CC3)c(C(F)(F)F)cc2=O)c1F] |
| | 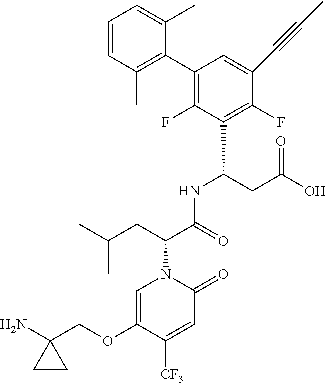 [SMILES: CC#Cc1cc(-c2c(C)cccc2C)c(F)c([C@H](CC(=O)O)NC(=O)[C@@H](CC(C)C)n2cc(OCC3(N)CC3)c(C(F)(F)F)cc2=O)c1F] |
| | 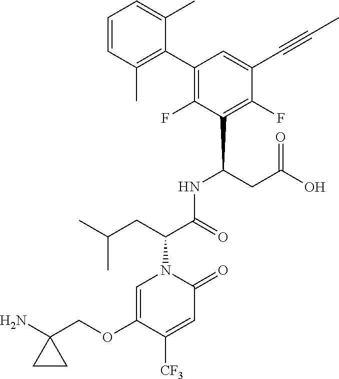 [SMILES: CC#Cc1cc(-c2c(C)cccc2C)c(F)c([C@@H](CC(=O)O)NC(=O)[C@@H](CC(C)C)n2cc(OCC3(N)CC3)c(C(F)(F)F)cc2=O)c1F] |
| 105 | 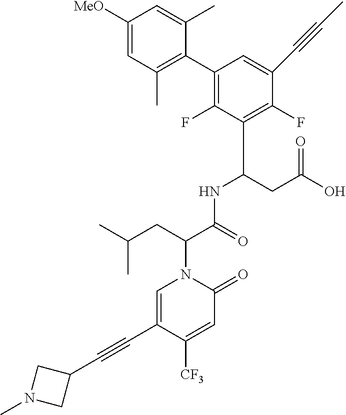 [SMILES: CC#Cc1cc(-c2c(C)cc(OC)cc2C)c(F)c(C(CC(=O)O)NC(=O)C(CC(C)C)n2cc(C#CC3CN(C)C3)c(C(F)(F)F)cc2=O)c1F] |
| | 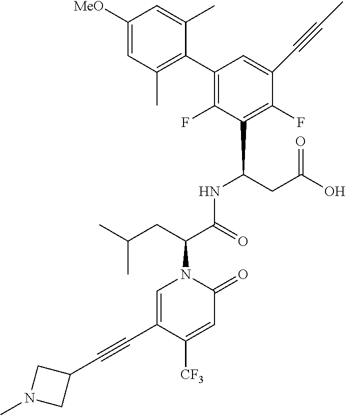 [SMILES: CC#Cc1cc(-c2c(C)cc(OC)cc2C)c(F)c([C@@H](CC(=O)O)NC(=O)[C@H](CC(C)C)n2cc(C#CC3CN(C)C3)c(C(F)(F)F)cc2=O)c1F] |
| | 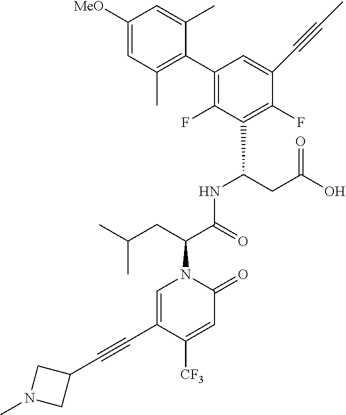 [SMILES: CC#Cc1cc(-c2c(C)cc(OC)cc2C)c(F)c([C@H](CC(=O)O)NC(=O)[C@H](CC(C)C)n2cc(C#CC3CN(C)C3)c(C(F)(F)F)cc2=O)c1F] |
| | 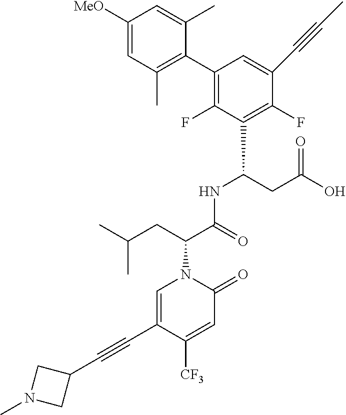 [SMILES: CC#Cc1cc(-c2c(C)cc(OC)cc2C)c(F)c([C@H](CC(=O)O)NC(=O)[C@@H](CC(C)C)n2cc(C#CC3CN(C)C3)c(C(F)(F)F)cc2=O)c1F] |
| | 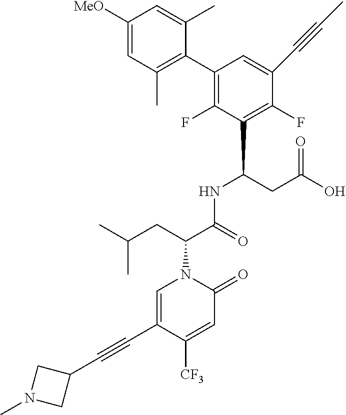 [SMILES: CC#Cc1cc(-c2c(C)cc(OC)cc2C)c(F)c([C@@H](CC(=O)O)NC(=O)[C@@H](CC(C)C)n2cc(C#CC3CN(C)C3)c(C(F)(F)F)cc2=O)c1F] |
| 106 | 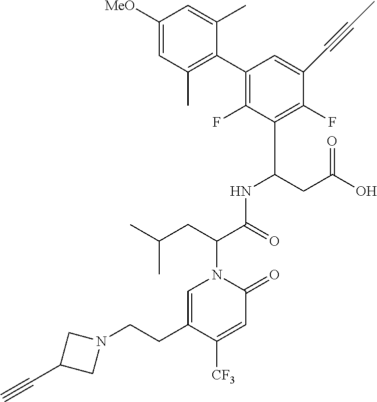 [SMILES: C#CC1CN(CCc2cn(C(CC(C)C)C(=O)NC(CC(=O)O)c3c(F)c(C#CC)cc(-c4c(C)cc(OC)cc4C)c3F)c(=O)cc2C(F)(F)F)C1] |
| | 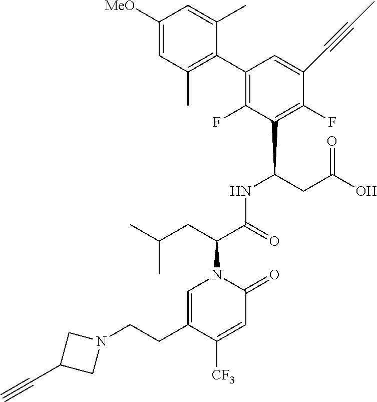 [SMILES: C#CC1CN(CCc2cn([C@@H](CC(C)C)C(=O)N[C@H](CC(=O)O)c3c(F)c(C#CC)cc(-c4c(C)cc(OC)cc4C)c3F)c(=O)cc2C(F)(F)F)C1] |
| | 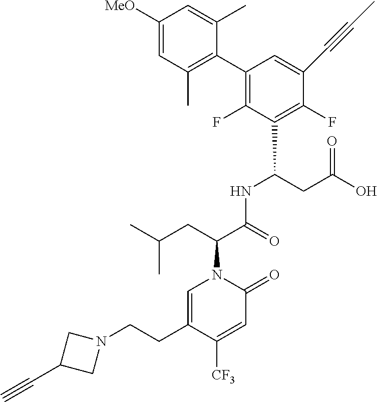 [SMILES: C#CC1CN(CCc2cn([C@@H](CC(C)C)C(=O)N[C@@H](CC(=O)O)c3c(F)c(C#CC)cc(-c4c(C)cc(OC)cc4C)c3F)c(=O)cc2C(F)(F)F)C1] |
| | 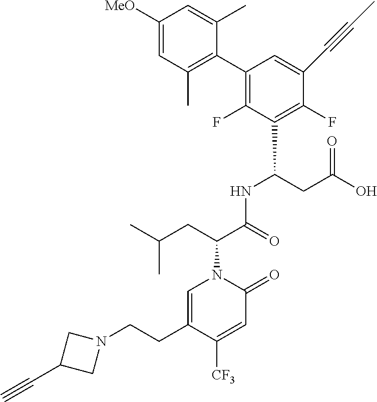 [SMILES: C#CC1CN(CCc2cn([C@H](CC(C)C)C(=O)N[C@@H](CC(=O)O)c3c(F)c(C#CC)cc(-c4c(C)cc(OC)cc4C)c3F)c(=O)cc2C(F)(F)F)C1] |
| | 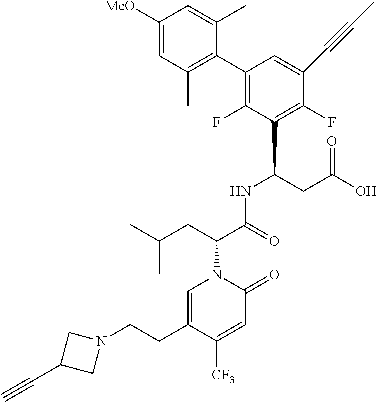 [SMILES: C#CC1CN(CCc2cn([C@H](CC(C)C)C(=O)N[C@H](CC(=O)O)c3c(F)c(C#CC)cc(-c4c(C)cc(OC)cc4C)c3F)c(=O)cc2C(F)(F)F)C1] |
| 107 | 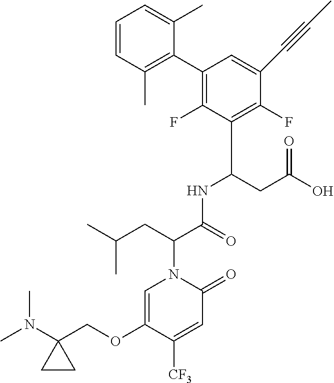 [SMILES: CC#Cc1cc(-c2c(C)cccc2C)c(F)c(C(CC(=O)O)NC(=O)C(CC(C)C)n2cc(OCC3(N(C)C)CC3)c(C(F)(F)F)cc2=O)c1F] |
| | 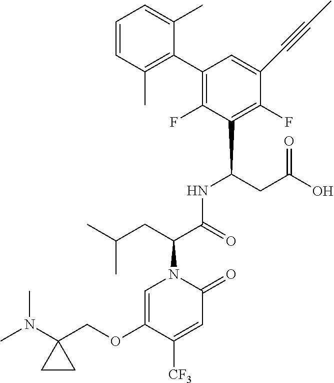 [SMILES: CC#Cc1cc(-c2c(C)cccc2C)c(F)c([C@@H](CC(=O)O)NC(=O)[C@H](CC(C)C)n2cc(OCC3(N(C)C)CC3)c(C(F)(F)F)cc2=O)c1F] |
| | 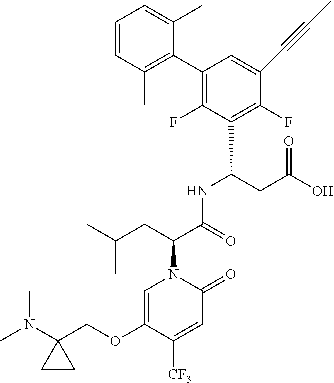 [SMILES: CC#Cc1cc(-c2c(C)cccc2C)c(F)c([C@H](CC(=O)O)NC(=O)[C@H](CC(C)C)n2cc(OCC3(N(C)C)CC3)c(C(F)(F)F)cc2=O)c1F] |
| | 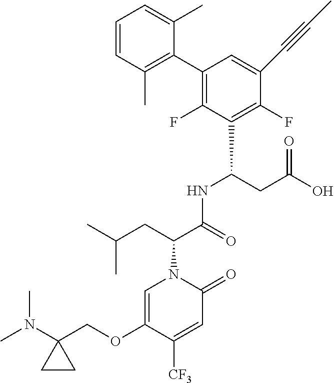 [SMILES: CC#Cc1cc(-c2c(C)cccc2C)c(F)c([C@H](CC(=O)O)NC(=O)[C@@H](CC(C)C)n2cc(OCC3(N(C)C)CC3)c(C(F)(F)F)cc2=O)c1F] |
| | 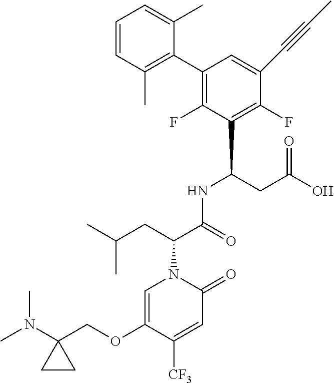 [SMILES: CC#Cc1cc(-c2c(C)cccc2C)c(F)c([C@@H](CC(=O)O)NC(=O)[C@@H](CC(C)C)n2cc(OCC3(N(C)C)CC3)c(C(F)(F)F)cc2=O)c1F] |
| 108 | 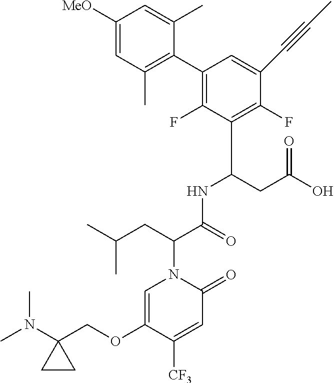 [SMILES: CC#Cc1cc(-c2c(C)cc(OC)cc2C)c(F)c(C(CC(=O)O)NC(=O)C(CC(C)C)n2cc(OCC3(N(C)C)CC3)c(C(F)(F)F)cc2=O)c1F] |
| | 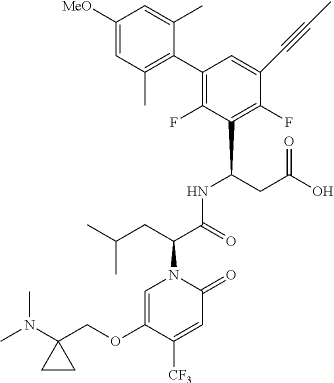 [SMILES: CC#Cc1cc(-c2c(C)cc(OC)cc2C)c(F)c([C@@H](CC(=O)O)NC(=O)[C@H](CC(C)C)n2cc(OCC3(N(C)C)CC3)c(C(F)(F)F)cc2=O)c1F] |
| | 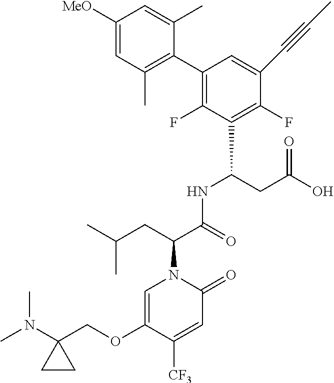 [SMILES: CC#Cc1cc(-c2c(C)cc(OC)cc2C)c(F)c([C@H](CC(=O)O)NC(=O)[C@H](CC(C)C)n2cc(OCC3(N(C)C)CC3)c(C(F)(F)F)cc2=O)c1F] |
| | 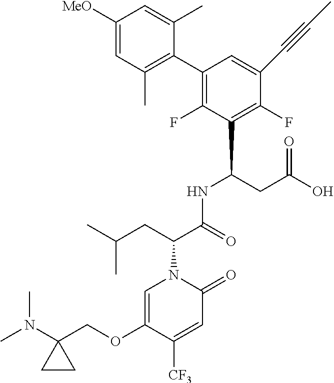 [SMILES: CC#Cc1cc(-c2c(C)cc(OC)cc2C)c(F)c([C@@H](CC(=O)O)NC(=O)[C@@H](CC(C)C)n2cc(OCC3(N(C)C)CC3)c(C(F)(F)F)cc2=O)c1F] |
| | 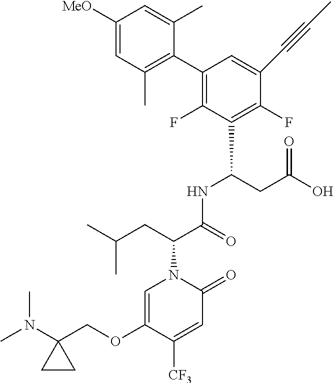 [SMILES: CC#Cc1cc(-c2c(C)cc(OC)cc2C)c(F)c([C@H](CC(=O)O)NC(=O)[C@@H](CC(C)C)n2cc(OCC3(N(C)C)CC3)c(C(F)(F)F)cc2=O)c1F] |
| 109 | 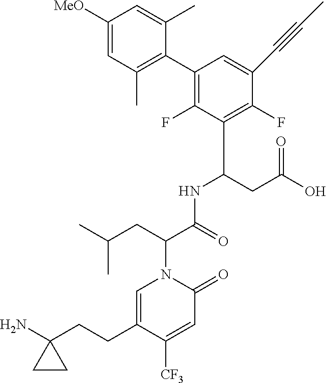 [SMILES: CC#Cc1cc(-c2c(C)cc(OC)cc2C)c(F)c(C(CC(=O)O)NC(=O)C(CC(C)C)n2cc(CCC3(N)CC3)c(C(F)(F)F)cc2=O)c1F] |
| | 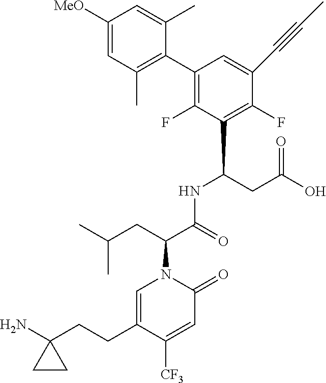 [SMILES: CC#Cc1cc(-c2c(C)cc(OC)cc2C)c(F)c([C@@H](CC(=O)O)NC(=O)[C@H](CC(C)C)n2cc(CCC3(N)CC3)c(C(F)(F)F)cc2=O)c1F] |
| | 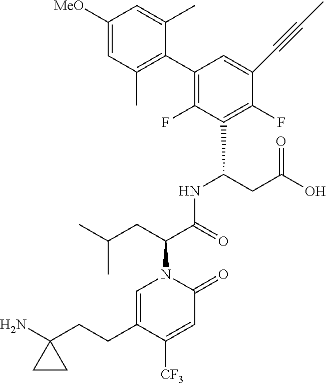 [SMILES: CC#Cc1cc(-c2c(C)cc(OC)cc2C)c(F)c([C@H](CC(=O)O)NC(=O)[C@H](CC(C)C)n2cc(CCC3(N)CC3)c(C(F)(F)F)cc2=O)c1F] |
| | 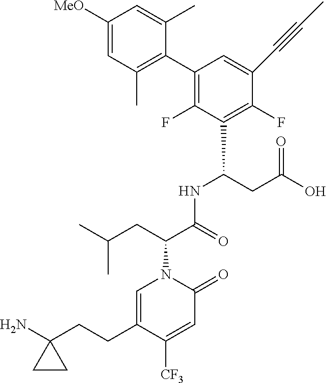 [SMILES: CC#Cc1cc(-c2c(C)cc(OC)cc2C)c(F)c([C@H](CC(=O)O)NC(=O)[C@@H](CC(C)C)n2cc(CCC3(N)CC3)c(C(F)(F)F)cc2=O)c1F] |
| | 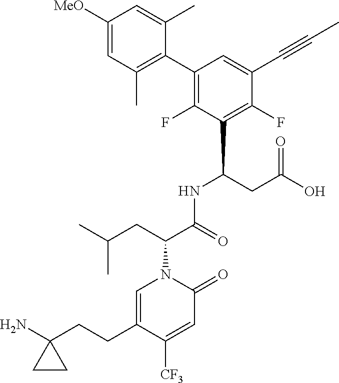 [SMILES: CC#Cc1cc(-c2c(C)cc(OC)cc2C)c(F)c([C@@H](CC(=O)O)NC(=O)[C@@H](CC(C)C)n2cc(CCC3(N)CC3)c(C(F)(F)F)cc2=O)c1F] |
| 110 | 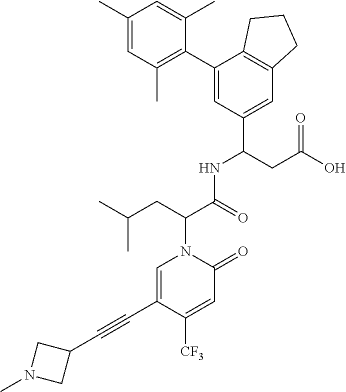 [SMILES: Cc1cc(C)c(-c2cc(C(CC(=O)O)NC(=O)C(CC(C)C)n3cc(C#CC4CN(C)C4)c(C(F)(F)F)cc3=O)cc3c2CCC3)c(C)c1] |
| | 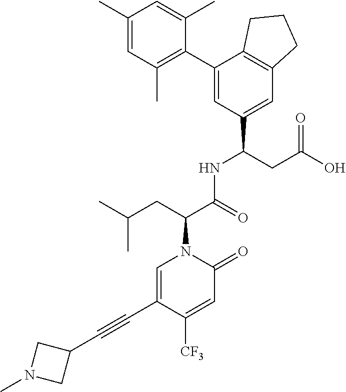 [SMILES: Cc1cc(C)c(-c2cc([C@@H](CC(=O)O)NC(=O)[C@H](CC(C)C)n3cc(C#CC4CN(C)C4)c(C(F)(F)F)cc3=O)cc3c2CCC3)c(C)c1] |
| | 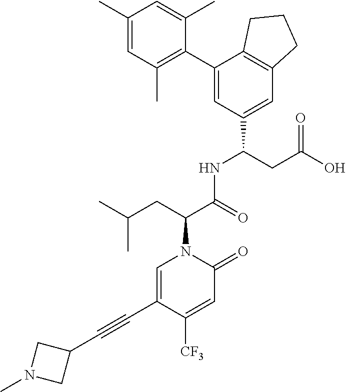 [SMILES: Cc1cc(C)c(-c2cc([C@H](CC(=O)O)NC(=O)[C@H](CC(C)C)n3cc(C#CC4CN(C)C4)c(C(F)(F)F)cc3=O)cc3c2CCC3)c(C)c1] |
| | 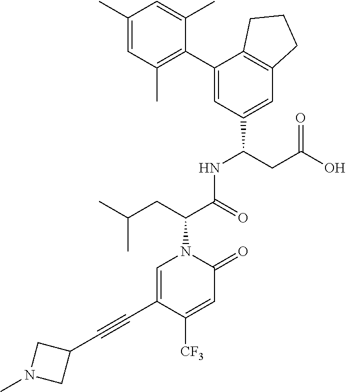 [SMILES: Cc1cc(C)c(-c2cc([C@H](CC(=O)O)NC(=O)[C@@H](CC(C)C)n3cc(C#CC4CN(C)C4)c(C(F)(F)F)cc3=O)cc3c2CCC3)c(C)c1] |
| | 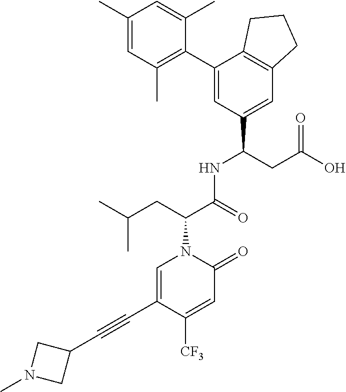 [SMILES: Cc1cc(C)c(-c2cc([C@@H](CC(=O)O)NC(=O)[C@@H](CC(C)C)n3cc(C#CC4CN(C)C4)c(C(F)(F)F)cc3=O)cc3c2CCC3)c(C)c1] |
| 111 | 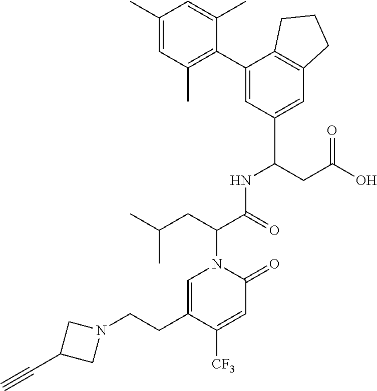 [SMILES: C#CC1CN(CCc2cn(C(CC(C)C)C(=O)NC(CC(=O)O)c3cc4c(c(-c5c(C)cc(C)cc5C)c3)CCC4)c(=O)cc2C(F)(F)F)C1] |
| | 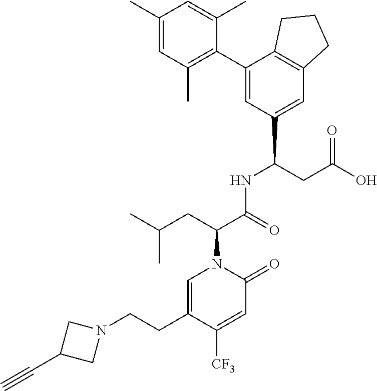 [SMILES: C#CC1CN(CCc2cn([C@@H](CC(C)C)C(=O)N[C@H](CC(=O)O)c3cc4c(c(-c5c(C)cc(C)cc5C)c3)CCC4)c(=O)cc2C(F)(F)F)C1] |
| | 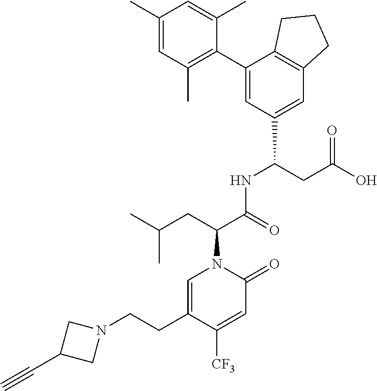 [SMILES: C#CC1CN(CCc2cn([C@@H](CC(C)C)C(=O)N[C@@H](CC(=O)O)c3cc4c(c(-c5c(C)cc(C)cc5C)c3)CCC4)c(=O)cc2C(F)(F)F)C1] |
| | 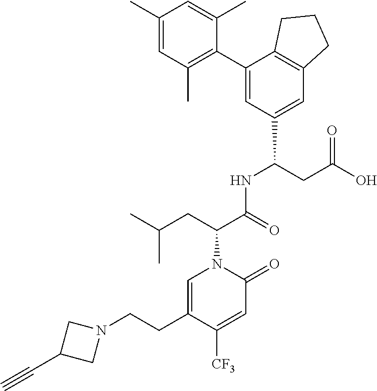 [SMILES: C#CC1CN(CCc2cn([C@H](CC(C)C)C(=O)N[C@@H](CC(=O)O)c3cc4c(c(-c5c(C)cc(C)cc5C)c3)CCC4)c(=O)cc2C(F)(F)F)C1] |
| | 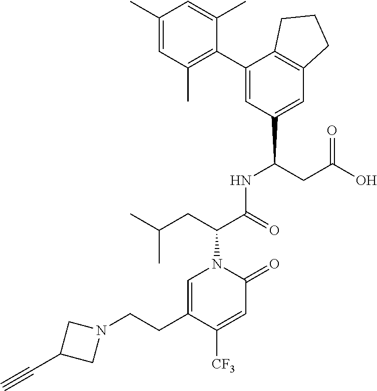 [SMILES: C#CC1CN(CCc2cn([C@H](CC(C)C)C(=O)N[C@H](CC(=O)O)c3cc4c(c(-c5c(C)cc(C)cc5C)c3)CCC4)c(=O)cc2C(F)(F)F)C1] |
| 112 | 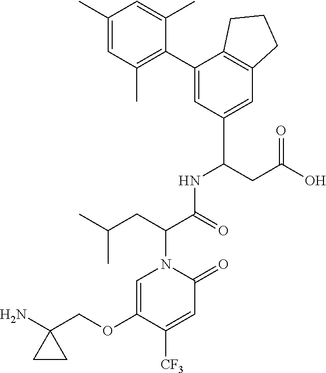 [SMILES: Cc1cc(C)c(-c2cc(C(CC(=O)O)NC(=O)C(CC(C)C)n3cc(OCC4(N)CC4)c(C(F)(F)F)cc3=O)cc3c2CCC3)c(C)c1] |
| | 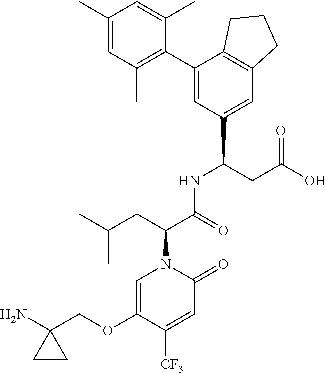 [SMILES: Cc1cc(C)c(-c2cc([C@@H](CC(=O)O)NC(=O)[C@H](CC(C)C)n3cc(OCC4(N)CC4)c(C(F)(F)F)cc3=O)cc3c2CCC3)c(C)c1] |
| | 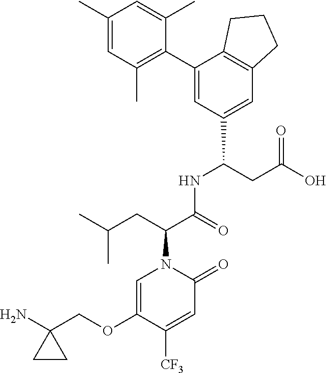 [SMILES: Cc1cc(C)c(-c2cc([C@H](CC(=O)O)NC(=O)[C@H](CC(C)C)n3cc(OCC4(N)CC4)c(C(F)(F)F)cc3=O)cc3c2CCC3)c(C)c1] |
| | 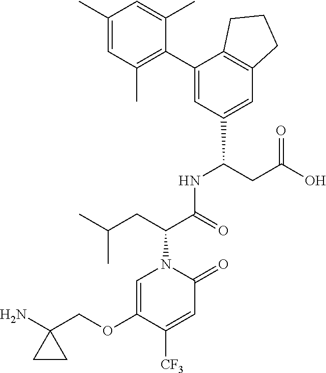 [SMILES: Cc1cc(C)c(-c2cc([C@H](CC(=O)O)NC(=O)[C@@H](CC(C)C)n3cc(OCC4(N)CC4)c(C(F)(F)F)cc3=O)cc3c2CCC3)c(C)c1] |
| | 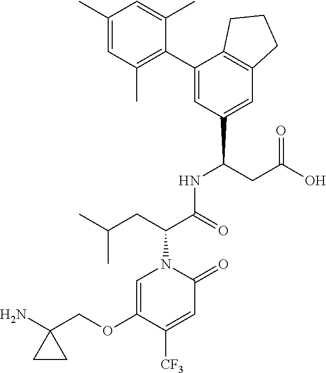 [SMILES: Cc1cc(C)c(-c2cc([C@@H](CC(=O)O)NC(=O)[C@@H](CC(C)C)n3cc(OCC4(N)CC4)c(C(F)(F)F)cc3=O)cc3c2CCC3)c(C)c1] |
| 113 | 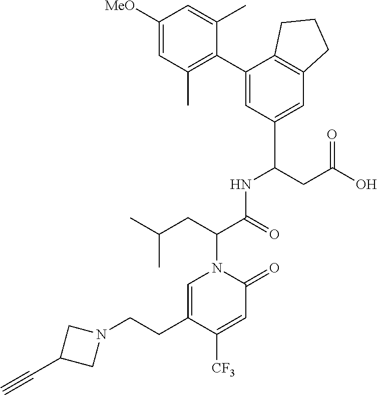 [SMILES: C#CC1CN(CCc2cn(C(CC(C)C)C(=O)NC(CC(=O)O)c3cc4c(c(-c5c(C)cc(OC)cc5C)c3)CCC4)c(=O)cc2C(F)(F)F)C1] |
| | 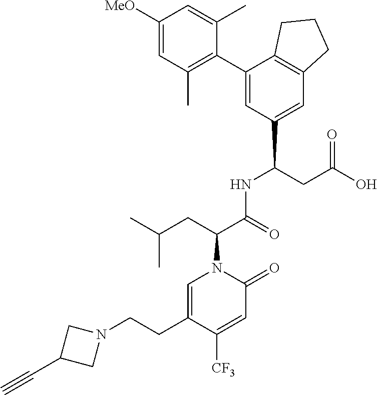 [SMILES: C#CC1CN(CCc2cn([C@@H](CC(C)C)C(=O)N[C@H](CC(=O)O)c3cc4c(c(-c5c(C)cc(OC)cc5C)c3)CCC4)c(=O)cc2C(F)(F)F)C1] |
| | 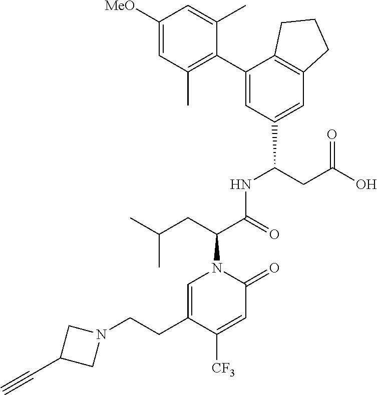 [SMILES: C#CC1CN(CCc2cn([C@@H](CC(C)C)C(=O)N[C@@H](CC(=O)O)c3cc4c(c(-c5c(C)cc(OC)cc5C)c3)CCC4)c(=O)cc2C(F)(F)F)C1] |
| | 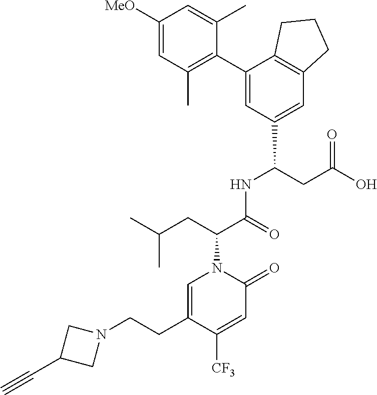 [SMILES: C#CC1CN(CCc2cn([C@H](CC(C)C)C(=O)N[C@@H](CC(=O)O)c3cc4c(c(-c5c(C)cc(OC)cc5C)c3)CCC4)c(=O)cc2C(F)(F)F)C1] |
| | 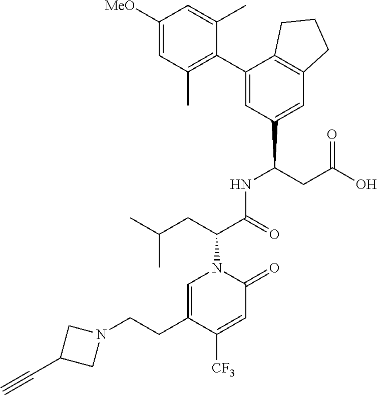 [SMILES: C#CC1CN(CCc2cn([C@H](CC(C)C)C(=O)N[C@H](CC(=O)O)c3cc4c(c(-c5c(C)cc(OC)cc5C)c3)CCC4)c(=O)cc2C(F)(F)F)C1] |
| 114 | 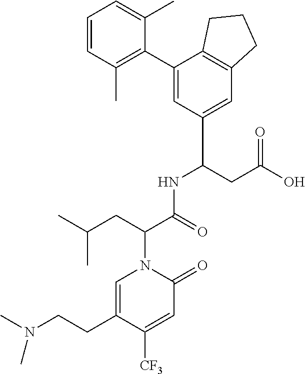 [SMILES: Cc1cccc(C)c1-c1cc(C(CC(=O)O)NC(=O)C(CC(C)C)n2cc(CCN(C)C)c(C(F)(F)F)cc2=O)cc2c1CCC2] |
| | 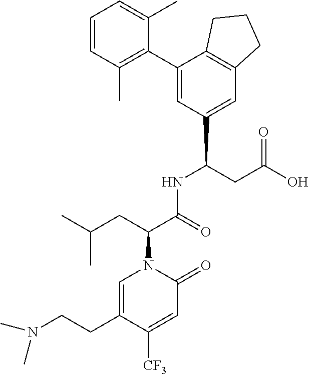 [SMILES: Cc1cccc(C)c1-c1cc([C@@H](CC(=O)O)NC(=O)[C@H](CC(C)C)n2cc(CCN(C)C)c(C(F)(F)F)cc2=O)cc2c1CCC2] |
| | 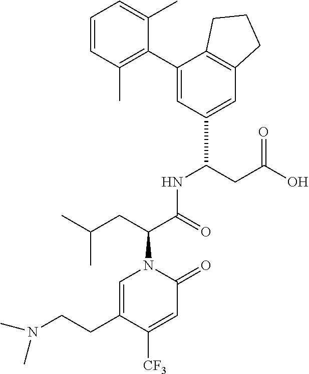 |
| | 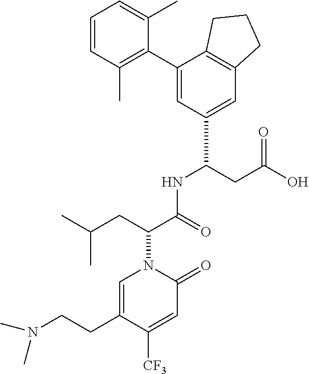 |
| | 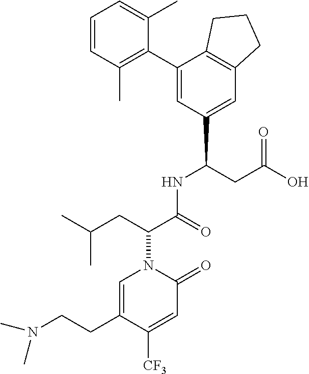 [SMILES: Cc1cccc(C)c1-c1cc([C@@H](CC(=O)O)NC(=O)[C@@H](CC(C)C)n2cc(CCN(C)C)c(C(F)(F)F)cc2=O)cc2c1CCC2] |
| 115 | 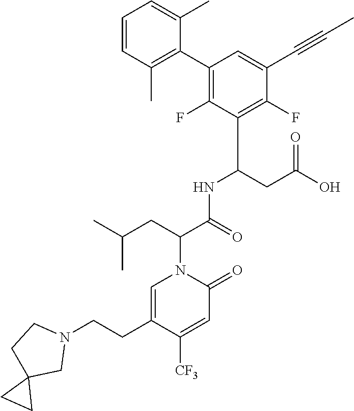 [SMILES: CC#Cc1cc(-c2c(C)cccc2C)c(F)c(C(CC(=O)O)NC(=O)C(CC(C)C)n2cc(CCN3CCC4(CC4)C3)c(C(F)(F)F)cc2=O)c1F] |
| | 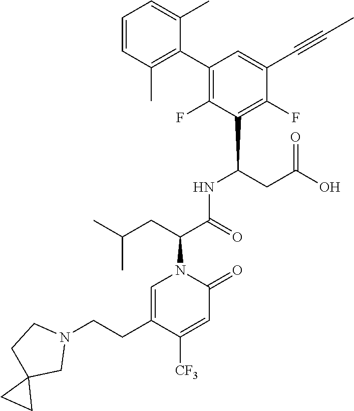 [SMILES: CC#Cc1cc(-c2c(C)cccc2C)c(F)c([C@@H](CC(=O)O)NC(=O)[C@H](CC(C)C)n2cc(CCN3CCC4(CC4)C3)c(C(F)(F)F)cc2=O)c1F] |
| | 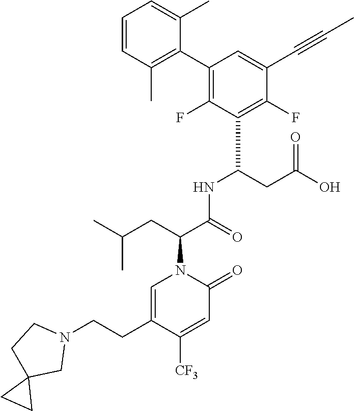 |
| | 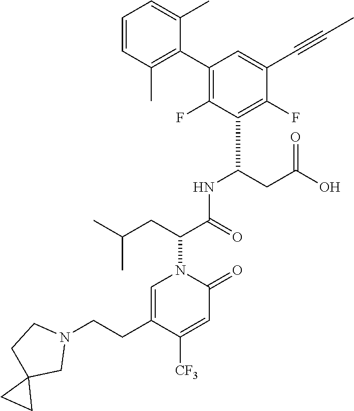 |
| | 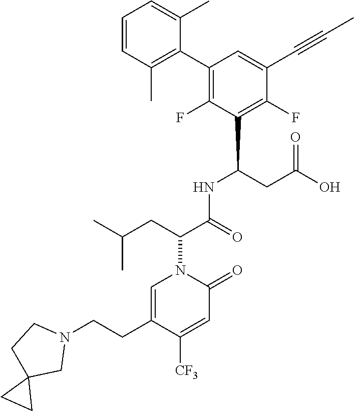 [SMILES: CC#Cc1cc(-c2c(C)cccc2C)c(F)c([C@@H](CC(=O)O)NC(=O)[C@@H](CC(C)C)n2cc(CCN3CCC4(CC4)C3)c(C(F)(F)F)cc2=O)c1F] |
| 116 | 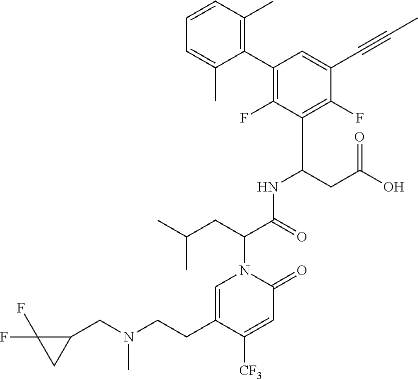 [SMILES: CC#Cc1cc(-c2c(C)cccc2C)c(F)c(C(CC(=O)O)NC(=O)C(CC(C)C)n2cc(CCN(C)CC3CC3(F)F)c(C(F)(F)F)cc2=O)c1F] |
| | 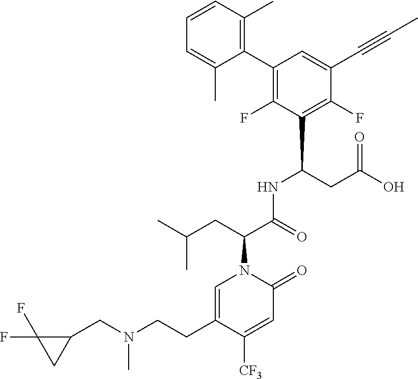 [SMILES: CC#Cc1cc(-c2c(C)cccc2C)c(F)c([C@@H](CC(=O)O)NC(=O)[C@H](CC(C)C)n2cc(CCN(C)CC3CC3(F)F)c(C(F)(F)F)cc2=O)c1F] |
| | 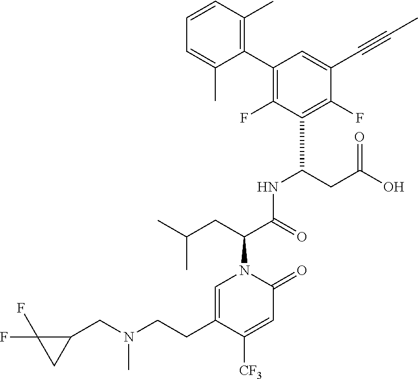 |
| | 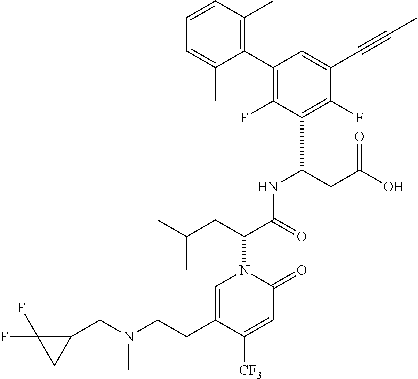 |
| | 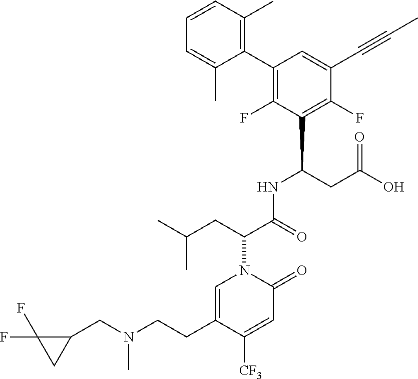 [SMILES: CC#Cc1cc(-c2c(C)cccc2C)c(F)c([C@@H](CC(=O)O)NC(=O)[C@@H](CC(C)C)n2cc(CCN(C)CC3CC3(F)F)c(C(F)(F)F)cc2=O)c1F] |
| 117 | 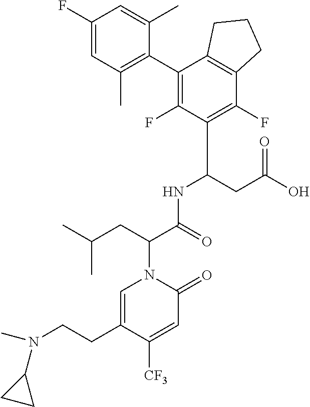 [SMILES: Cc1cc(F)cc(C)c1-c1c(F)c(C(CC(=O)O)NC(=O)C(CC(C)C)n2cc(CCN(C)C3CC3)c(C(F)(F)F)cc2=O)c(F)c2c1CCC2] |
| | 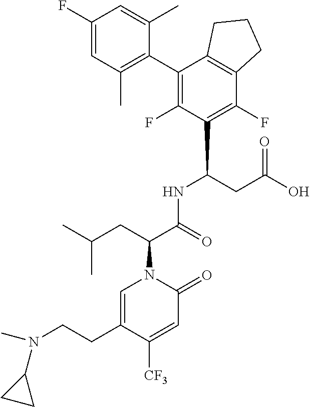 [SMILES: Cc1cc(F)cc(C)c1-c1c(F)c([C@@H](CC(=O)O)NC(=O)[C@H](CC(C)C)n2cc(CCN(C)C3CC3)c(C(F)(F)F)cc2=O)c(F)c2c1CCC2] |
| | 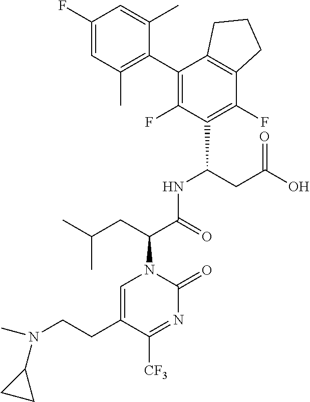 [SMILES: Cc1cc(F)cc(C)c1-c1c(F)c([C@H](CC(=O)O)NC(=O)[C@H](CC(C)C)n2cc(CCN(C)C3CC3)c(C(F)(F)F)nc2=O)c(F)c2c1CCC2] |
| | 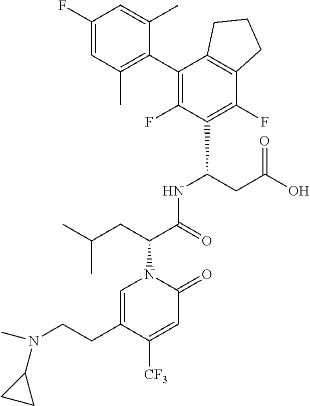 [SMILES: Cc1cc(F)cc(C)c1-c1c(F)c([C@H](CC(=O)O)NC(=O)[C@@H](CC(C)C)n2cc(CCN(C)C3CC3)c(C(F)(F)F)cc2=O)c(F)c2c1CCC2] |
| | 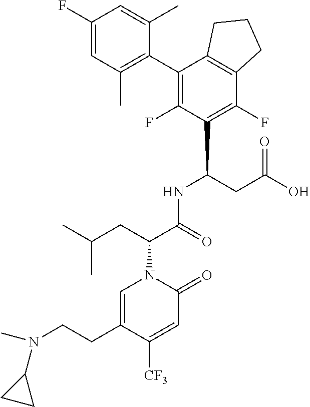 [SMILES: Cc1cc(F)cc(C)c1-c1c(F)c([C@@H](CC(=O)O)NC(=O)[C@@H](CC(C)C)n2cc(CCN(C)C3CC3)c(C(F)(F)F)cc2=O)c(F)c2c1CCC2] |
| 118 | 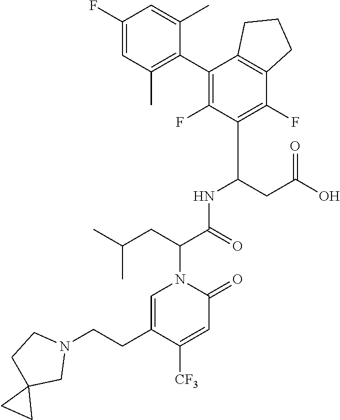 [SMILES: Cc1cc(F)cc(C)c1-c1c(F)c(C(CC(=O)O)NC(=O)C(CC(C)C)n2cc(CCN3CCC4(CC4)C3)c(C(F)(F)F)cc2=O)c(F)c2c1CCC2] |
| | 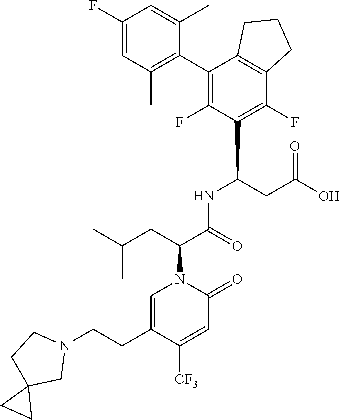 [SMILES: Cc1cc(F)cc(C)c1-c1c(F)c([C@@H](CC(=O)O)NC(=O)[C@H](CC(C)C)n2cc(CCN3CCC4(CC4)C3)c(C(F)(F)F)cc2=O)c(F)c2c1CCC2] |
| | 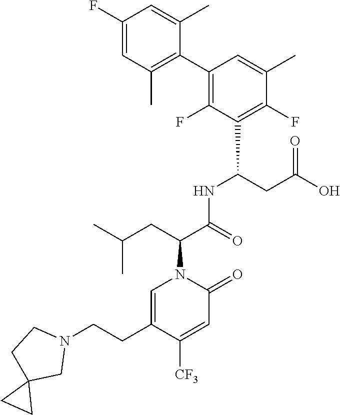 [SMILES: Cc1cc(-c2c(C)cc(F)cc2C)c(F)c([C@H](CC(=O)O)NC(=O)[C@H](CC(C)C)n2cc(CCN3CCC4(CC4)C3)c(C(F)(F)F)cc2=O)c1F] |
| | 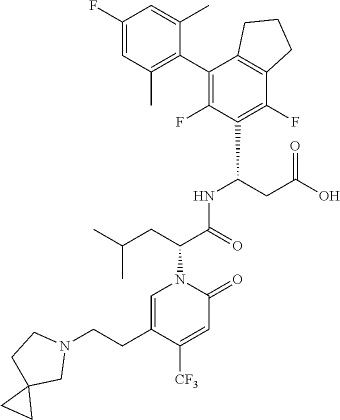 [SMILES: Cc1cc(F)cc(C)c1-c1c(F)c([C@H](CC(=O)O)NC(=O)[C@@H](CC(C)C)n2cc(CCN3CCC4(CC4)C3)c(C(F)(F)F)cc2=O)c(F)c2c1CCC2] |
| | 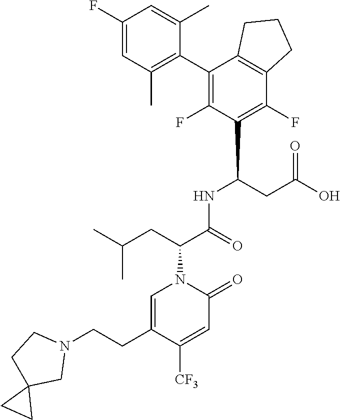 [SMILES: Cc1cc(F)cc(C)c1-c1c(F)c([C@@H](CC(=O)O)NC(=O)[C@@H](CC(C)C)n2cc(CCN3CCC4(CC4)C3)c(C(F)(F)F)cc2=O)c(F)c2c1CCC2] |
| 119 | 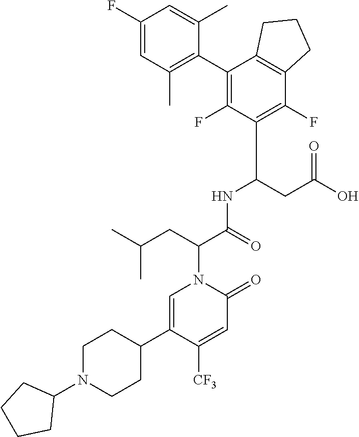 [SMILES: Cc1cc(F)cc(C)c1-c1c(F)c(C(CC(=O)O)NC(=O)C(CC(C)C)n2cc(C3CCN(C4CCCC4)CC3)c(C(F)(F)F)cc2=O)c(F)c2c1CCC2] |
| | 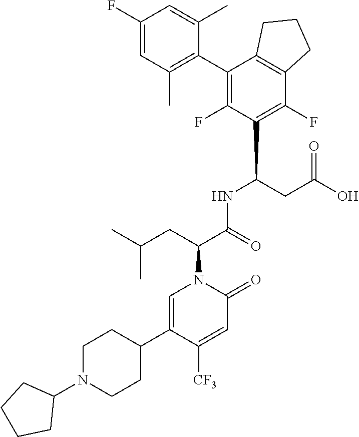 [SMILES: Cc1cc(F)cc(C)c1-c1c(F)c([C@@H](CC(=O)O)NC(=O)[C@H](CC(C)C)n2cc(C3CCN(C4CCCC4)CC3)c(C(F)(F)F)cc2=O)c(F)c2c1CCC2] |
| | 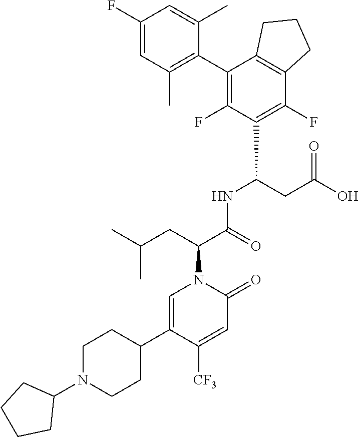 [SMILES: Cc1cc(F)cc(C)c1-c1c(F)c([C@H](CC(=O)O)NC(=O)[C@H](CC(C)C)n2cc(C3CCN(C4CCCC4)CC3)c(C(F)(F)F)cc2=O)c(F)c2c1CCC2] |
| | 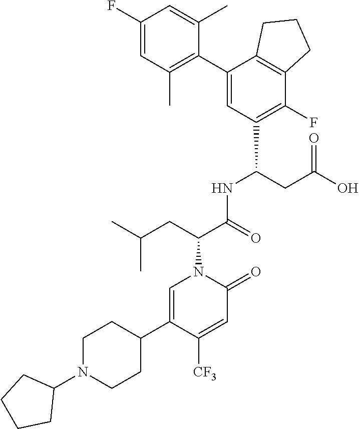 [SMILES: Cc1cc(F)cc(C)c1-c1cc([C@H](CC(=O)O)NC(=O)[C@@H](CC(C)C)n2cc(C3CCN(C4CCCC4)CC3)c(C(F)(F)F)cc2=O)c(F)c2c1CCC2] |
| | 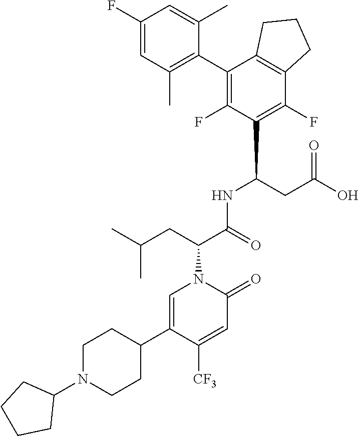 [SMILES: Cc1cc(F)cc(C)c1-c1c(F)c([C@@H](CC(=O)O)NC(=O)[C@@H](CC(C)C)n2cc(C3CCN(C4CCCC4)CC3)c(C(F)(F)F)cc2=O)c(F)c2c1CCC2] |
| 120 | 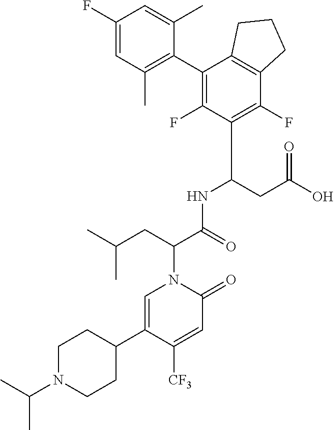 [SMILES: Cc1cc(F)cc(C)c1-c1c(F)c(C(CC(=O)O)NC(=O)C(CC(C)C)n2cc(C3CCN(C(C)C)CC3)c(C(F)(F)F)cc2=O)c(F)c2c1CCC2] |
| | 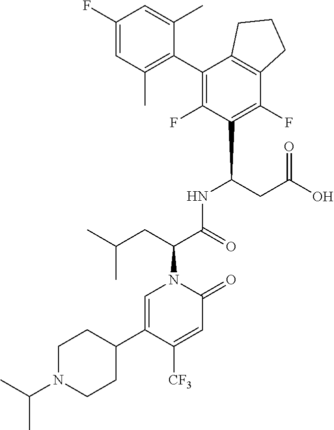 [SMILES: Cc1cc(F)cc(C)c1-c1c(F)c([C@@H](CC(=O)O)NC(=O)[C@H](CC(C)C)n2cc(C3CCN(C(C)C)CC3)c(C(F)(F)F)cc2=O)c(F)c2c1CCC2] |
| | 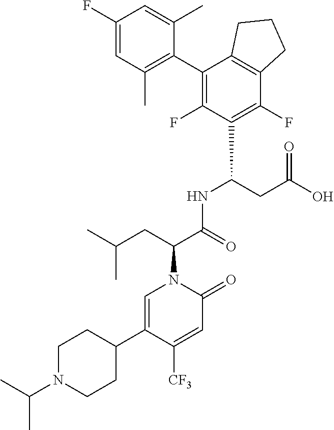 [SMILES: Cc1cc(F)cc(C)c1-c1c(F)c([C@H](CC(=O)O)NC(=O)[C@H](CC(C)C)n2cc(C3CCN(C(C)C)CC3)c(C(F)(F)F)cc2=O)c(F)c2c1CCC2] |
| | 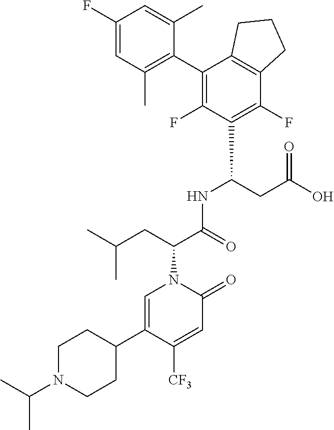 [SMILES: Cc1cc(F)cc(C)c1-c1c(F)c([C@H](CC(=O)O)NC(=O)[C@@H](CC(C)C)n2cc(C3CCN(C(C)C)CC3)c(C(F)(F)F)cc2=O)c(F)c2c1CCC2] |
| | 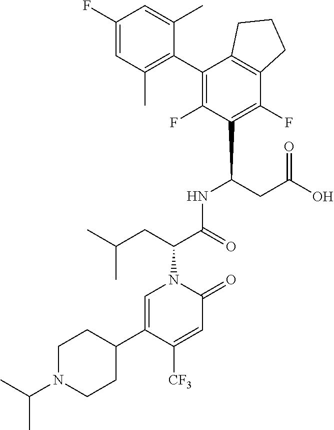 [SMILES: Cc1cc(F)cc(C)c1-c1c(F)c([C@@H](CC(=O)O)NC(=O)[C@@H](CC(C)C)n2cc(C3CCN(C(C)C)CC3)c(C(F)(F)F)cc2=O)c(F)c2c1CCC2] |
| 121 | 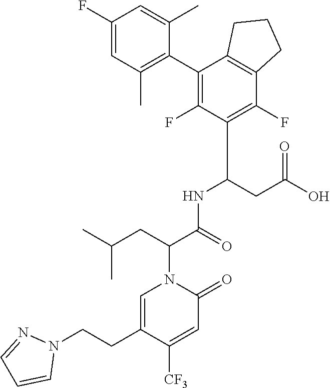 [SMILES: Cc1cc(F)cc(C)c1-c1c(F)c(C(CC(=O)O)NC(=O)C(CC(C)C)n2cc(CCn3cccn3)c(C(F)(F)F)cc2=O)c(F)c2c1CCC2] |
| | 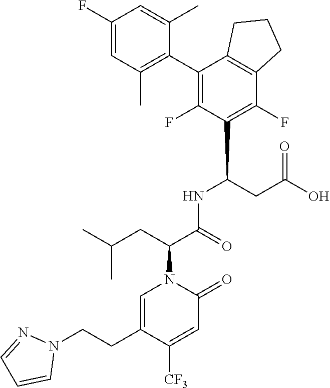 [SMILES: Cc1cc(F)cc(C)c1-c1c(F)c([C@@H](CC(=O)O)NC(=O)[C@H](CC(C)C)n2cc(CCn3cccn3)c(C(F)(F)F)cc2=O)c(F)c2c1CCC2] |
| | 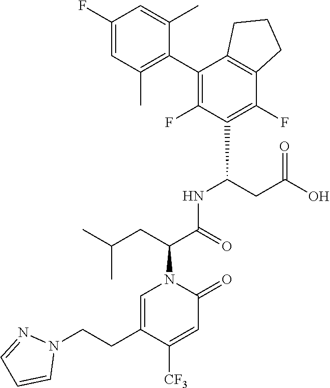 [SMILES: Cc1cc(F)cc(C)c1-c1c(F)c([C@H](CC(=O)O)NC(=O)[C@H](CC(C)C)n2cc(CCn3cccn3)c(C(F)(F)F)cc2=O)c(F)c2c1CCC2] |
| | 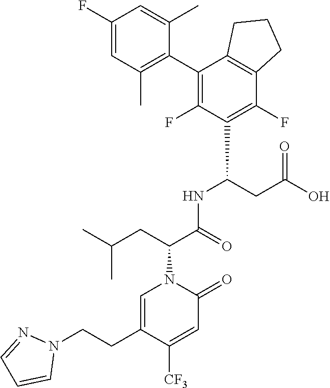 [SMILES: Cc1cc(F)cc(C)c1-c1c(F)c([C@H](CC(=O)O)NC(=O)[C@@H](CC(C)C)n2cc(CCn3cccn3)c(C(F)(F)F)cc2=O)c(F)c2c1CCC2] |
| | 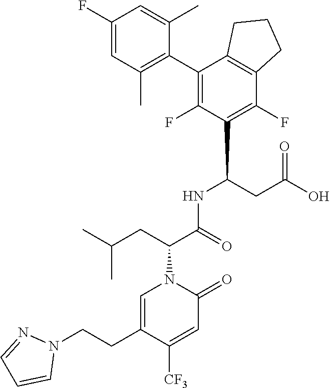 [SMILES: Cc1cc(F)cc(C)c1-c1c(F)c([C@@H](CC(=O)O)NC(=O)[C@@H](CC(C)C)n2cc(CCn3cccn3)c(C(F)(F)F)cc2=O)c(F)c2c1CCC2] |
| 122 | 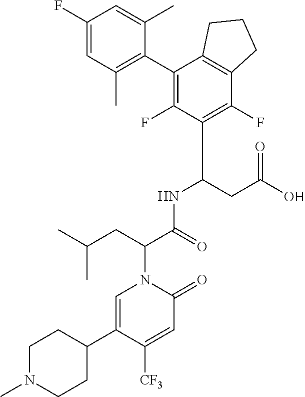 [SMILES: Cc1cc(F)cc(C)c1-c1c(F)c(C(CC(=O)O)NC(=O)C(CC(C)C)n2cc(C3CCN(C)CC3)c(C(F)(F)F)cc2=O)c(F)c2c1CCC2] |
| | 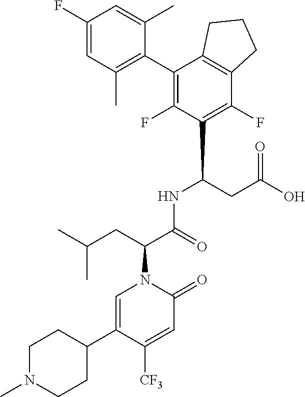 [SMILES: Cc1cc(F)cc(C)c1-c1c(F)c([C@@H](CC(=O)O)NC(=O)[C@H](CC(C)C)n2cc(C3CCN(C)CC3)c(C(F)(F)F)cc2=O)c(F)c2c1CCC2] |
| | 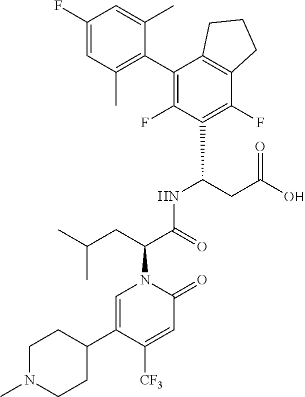 [SMILES: Cc1cc(F)cc(C)c1-c1c(F)c([C@H](CC(=O)O)NC(=O)[C@H](CC(C)C)n2cc(C3CCN(C)CC3)c(C(F)(F)F)cc2=O)c(F)c2c1CCC2] |
| | 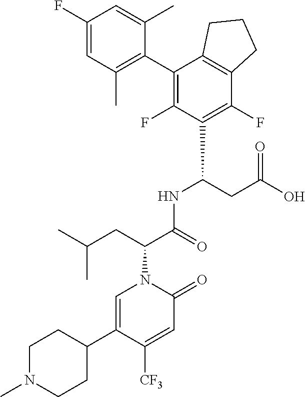 [SMILES: Cc1cc(F)cc(C)c1-c1c(F)c([C@H](CC(=O)O)NC(=O)[C@@H](CC(C)C)n2cc(C3CCN(C)CC3)c(C(F)(F)F)cc2=O)c(F)c2c1CCC2] |
| | 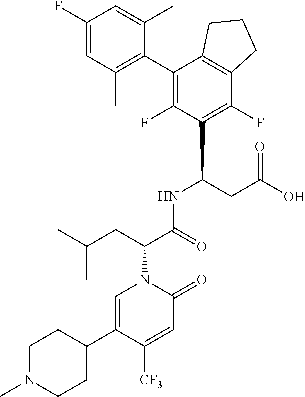 [SMILES: Cc1cc(F)cc(C)c1-c1c(F)c([C@@H](CC(=O)O)NC(=O)[C@@H](CC(C)C)n2cc(C3CCN(C)CC3)c(C(F)(F)F)cc2=O)c(F)c2c1CCC2] |
| 123 | 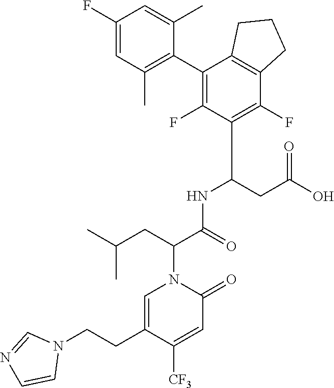 [SMILES: Cc1cc(F)cc(C)c1-c1c(F)c(C(CC(=O)O)NC(=O)C(CC(C)C)n2cc(CCn3ccnc3)c(C(F)(F)F)cc2=O)c(F)c2c1CCC2] |
| | 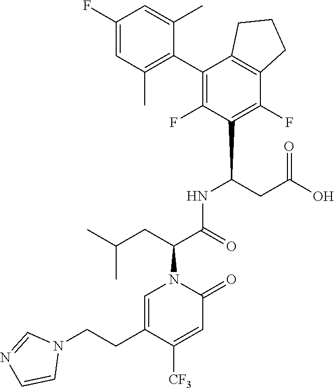 [SMILES: Cc1cc(F)cc(C)c1-c1c(F)c([C@@H](CC(=O)O)NC(=O)[C@H](CC(C)C)n2cc(CCn3ccnc3)c(C(F)(F)F)cc2=O)c(F)c2c1CCC2] |
| | 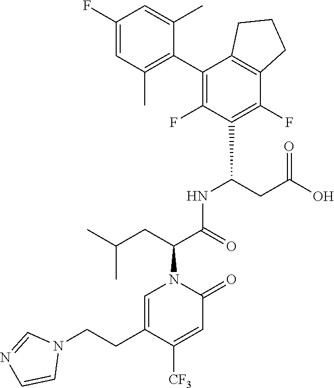 [SMILES: Cc1cc(F)cc(C)c1-c1c(F)c([C@H](CC(=O)O)NC(=O)[C@H](CC(C)C)n2cc(CCn3ccnc3)c(C(F)(F)F)cc2=O)c(F)c2c1CCC2] |
| | 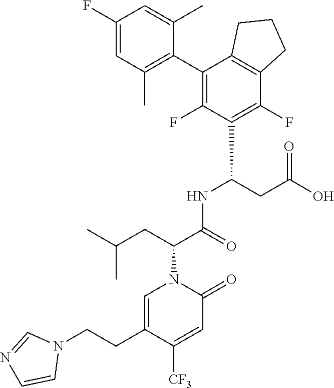 [SMILES: Cc1cc(F)cc(C)c1-c1c(F)c([C@H](CC(=O)O)NC(=O)[C@@H](CC(C)C)n2cc(CCn3ccnc3)c(C(F)(F)F)cc2=O)c(F)c2c1CCC2] |
| | 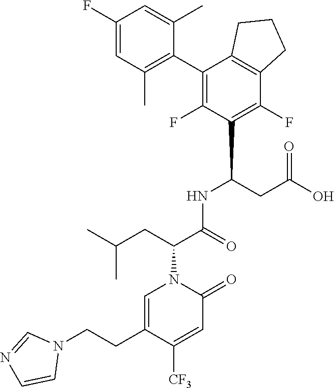 [SMILES: Cc1cc(F)cc(C)c1-c1c(F)c([C@@H](CC(=O)O)NC(=O)[C@@H](CC(C)C)n2cc(CCn3ccnc3)c(C(F)(F)F)cc2=O)c(F)c2c1CCC2] |
| 124 | 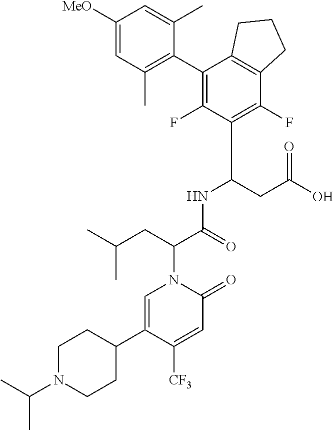 [SMILES: COc1cc(C)c(-c2c(F)c(C(CC(=O)O)NC(=O)C(CC(C)C)n3cc(C4CCN(C(C)C)CC4)c(C(F)(F)F)cc3=O)c(F)c3c2CCC3)c(C)c1] |
| | 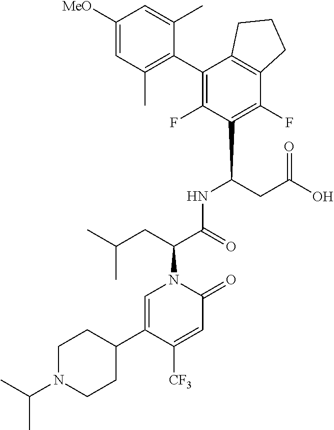 [SMILES: COc1cc(C)c(-c2c(F)c([C@@H](CC(=O)O)NC(=O)[C@H](CC(C)C)n3cc(C4CCN(C(C)C)CC4)c(C(F)(F)F)cc3=O)c(F)c3c2CCC3)c(C)c1] |
| | 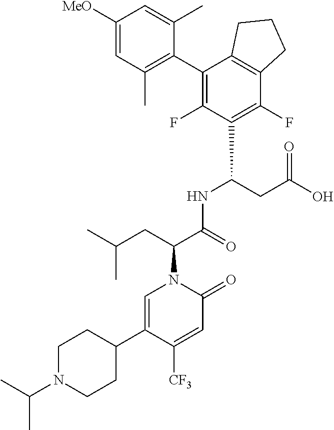 [SMILES: COc1cc(C)c(-c2c(F)c([C@H](CC(=O)O)NC(=O)[C@H](CC(C)C)n3cc(C4CCN(C(C)C)CC4)c(C(F)(F)F)cc3=O)c(F)c3c2CCC3)c(C)c1] |
| | 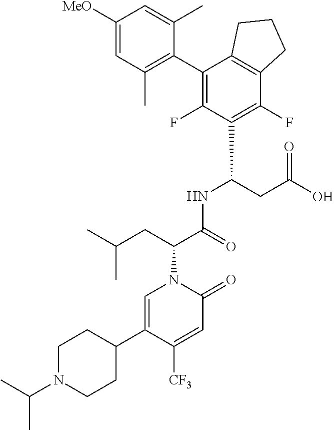 [SMILES: COc1cc(C)c(-c2c(F)c([C@H](CC(=O)O)NC(=O)[C@@H](CC(C)C)n3cc(C4CCN(C(C)C)CC4)c(C(F)(F)F)cc3=O)c(F)c3c2CCC3)c(C)c1] |
| | 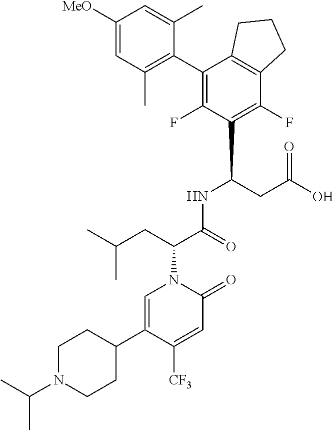 [SMILES: COc1cc(C)c(-c2c(F)c([C@@H](CC(=O)O)NC(=O)[C@@H](CC(C)C)n3cc(C4CCN(C(C)C)CC4)c(C(F)(F)F)cc3=O)c(F)c3c2CCC3)c(C)c1] |
| 125 | 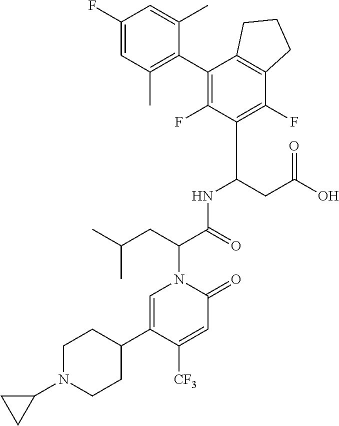 [SMILES: Cc1cc(F)cc(C)c1-c1c(F)c(C(CC(=O)O)NC(=O)C(CC(C)C)n2cc(C3CCN(C4CC4)CC3)c(C(F)(F)F)cc2=O)c(F)c2c1CCC2] |
| | 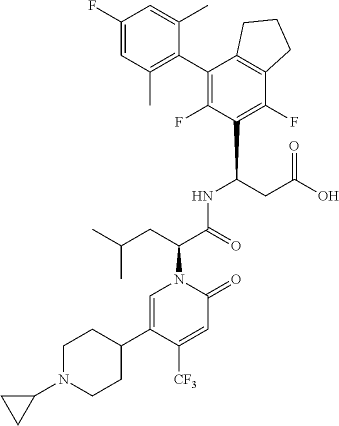 [SMILES: Cc1cc(F)cc(C)c1-c1c(F)c([C@@H](CC(=O)O)NC(=O)[C@H](CC(C)C)n2cc(C3CCN(C4CC4)CC3)c(C(F)(F)F)cc2=O)c(F)c2c1CCC2] |
| | 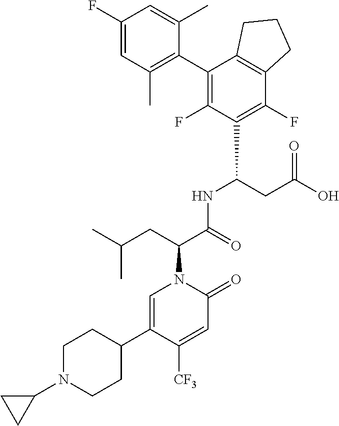 [SMILES: Cc1cc(F)cc(C)c1-c1c(F)c([C@H](CC(=O)O)NC(=O)[C@H](CC(C)C)n2cc(C3CCN(C4CC4)CC3)c(C(F)(F)F)cc2=O)c(F)c2c1CCC2] |
| | 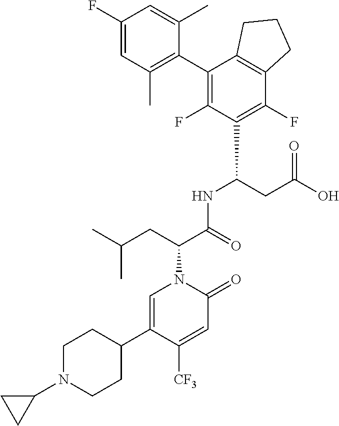 [SMILES: Cc1cc(F)cc(C)c1-c1c(F)c([C@H](CC(=O)O)NC(=O)[C@@H](CC(C)C)n2cc(C3CCN(C4CC4)CC3)c(C(F)(F)F)cc2=O)c(F)c2c1CCC2] |
| | 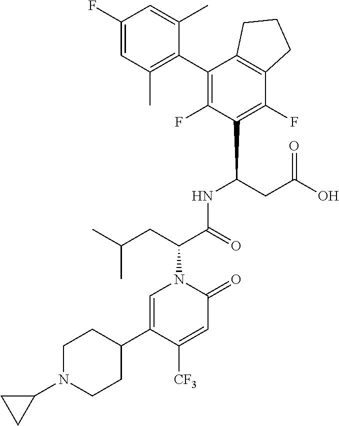 [SMILES: Cc1cc(F)cc(C)c1-c1c(F)c([C@@H](CC(=O)O)NC(=O)[C@@H](CC(C)C)n2cc(C3CCN(C4CC4)CC3)c(C(F)(F)F)cc2=O)c(F)c2c1CCC2] |
| 126 | 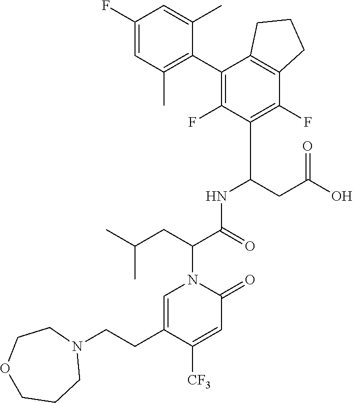 [SMILES: Cc1cc(F)cc(C)c1-c1c(F)c(C(CC(=O)O)NC(=O)C(CC(C)C)n2cc(CCN3CCCOCC3)c(C(F)(F)F)cc2=O)c(F)c2c1CCC2] |
| | 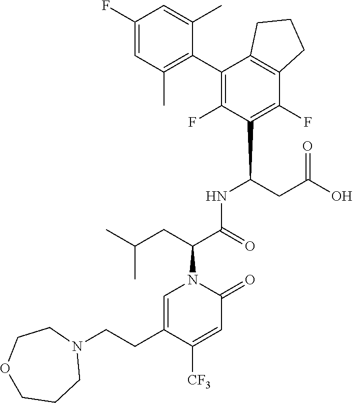 [SMILES: Cc1cc(F)cc(C)c1-c1c(F)c([C@@H](CC(=O)O)NC(=O)[C@H](CC(C)C)n2cc(CCN3CCCOCC3)c(C(F)(F)F)cc2=O)c(F)c2c1CCC2] |
| | 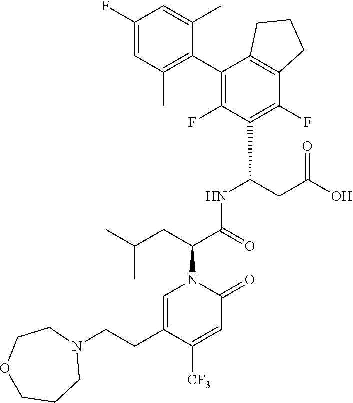 [SMILES: Cc1cc(F)cc(C)c1-c1c(F)c([C@H](CC(=O)O)NC(=O)[C@H](CC(C)C)n2cc(CCN3CCCOCC3)c(C(F)(F)F)cc2=O)c(F)c2c1CCC2] |
| | 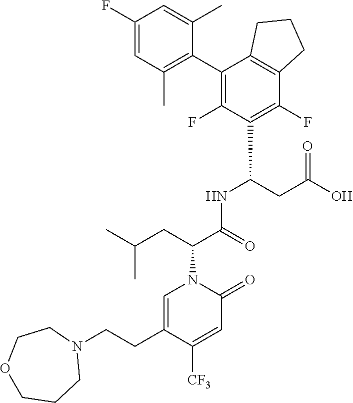 [SMILES: Cc1cc(F)cc(C)c1-c1c(F)c([C@H](CC(=O)O)NC(=O)[C@@H](CC(C)C)n2cc(CCN3CCCOCC3)c(C(F)(F)F)cc2=O)c(F)c2c1CCC2] |
| | 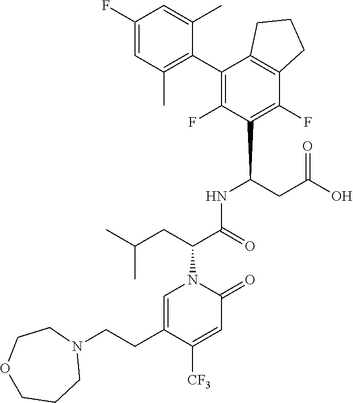 [SMILES: Cc1cc(F)cc(C)c1-c1c(F)c([C@@H](CC(=O)O)NC(=O)[C@@H](CC(C)C)n2cc(CCN3CCCOCC3)c(C(F)(F)F)cc2=O)c(F)c2c1CCC2] |
| 127 | 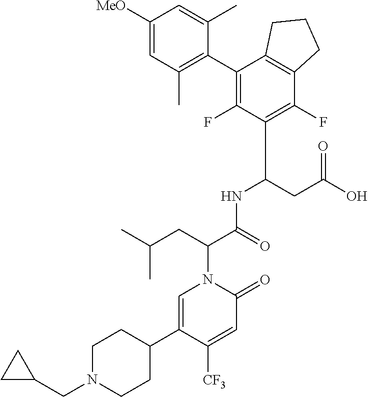 [SMILES: COc1cc(C)c(-c2c(F)c(C(CC(=O)O)NC(=O)C(CC(C)C)n3cc(C4CCN(CC5CC5)CC4)c(C(F)(F)F)cc3=O)c(F)c3c2CCC3)c(C)c1] |
| | 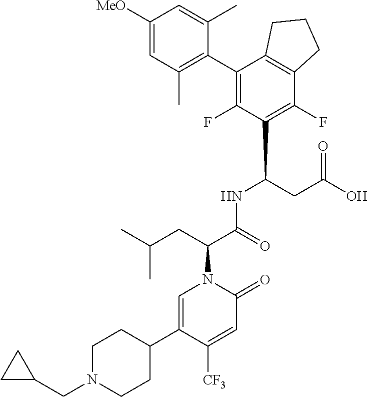 [SMILES: COc1cc(C)c(-c2c(F)c([C@@H](CC(=O)O)NC(=O)[C@H](CC(C)C)n3cc(C4CCN(CC5CC5)CC4)c(C(F)(F)F)cc3=O)c(F)c3c2CCC3)c(C)c1] |
| | 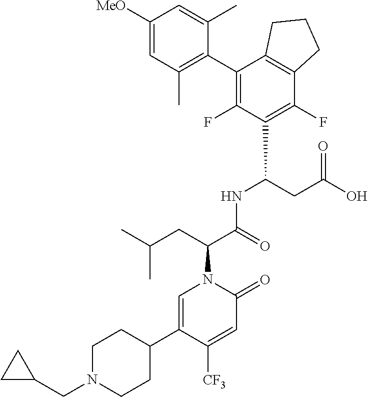 [SMILES: COc1cc(C)c(-c2c(F)c([C@H](CC(=O)O)NC(=O)[C@H](CC(C)C)n3cc(C4CCN(CC5CC5)CC4)c(C(F)(F)F)cc3=O)c(F)c3c2CCC3)c(C)c1] |
| | 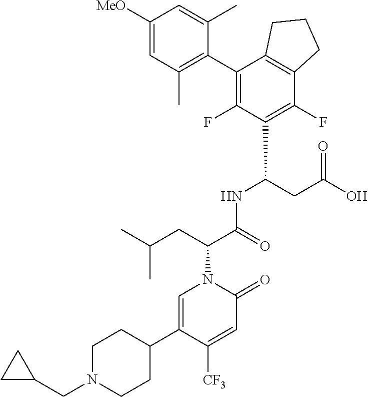 [SMILES: COc1cc(C)c(-c2c(F)c([C@H](CC(=O)O)NC(=O)[C@@H](CC(C)C)n3cc(C4CCN(CC5CC5)CC4)c(C(F)(F)F)cc3=O)c(F)c3c2CCC3)c(C)c1] |
| | 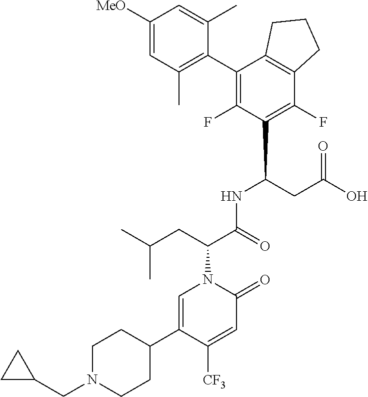 [SMILES: COc1cc(C)c(-c2c(F)c([C@@H](CC(=O)O)NC(=O)[C@@H](CC(C)C)n3cc(C4CCN(CC5CC5)CC4)c(C(F)(F)F)cc3=O)c(F)c3c2CCC3)c(C)c1] |
| 128 | 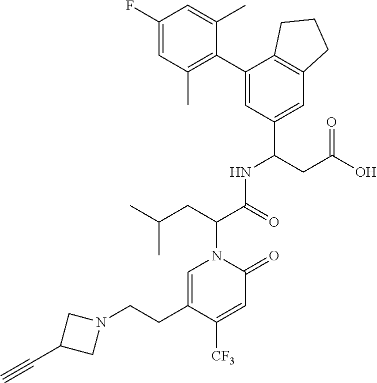 [SMILES: C#CC1CN(CCc2cn(C(CC(C)C)C(=O)NC(CC(=O)O)c3cc4c(c(-c5c(C)cc(F)cc5C)c3)CCC4)c(=O)cc2C(F)(F)F)C1] |
| | 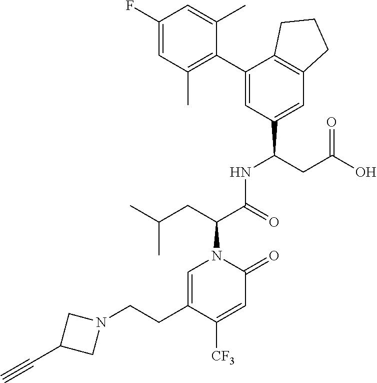 [SMILES: C#CC1CN(CCc2cn([C@@H](CC(C)C)C(=O)N[C@H](CC(=O)O)c3cc4c(c(-c5c(C)cc(F)cc5C)c3)CCC4)c(=O)cc2C(F)(F)F)C1] |
| | 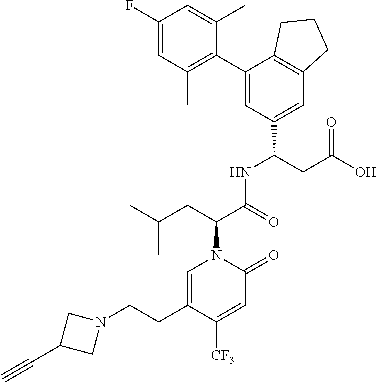 [SMILES: C#CC1CN(CCc2cn([C@@H](CC(C)C)C(=O)N[C@@H](CC(=O)O)c3cc4c(c(-c5c(C)cc(F)cc5C)c3)CCC4)c(=O)cc2C(F)(F)F)C1] |
| | 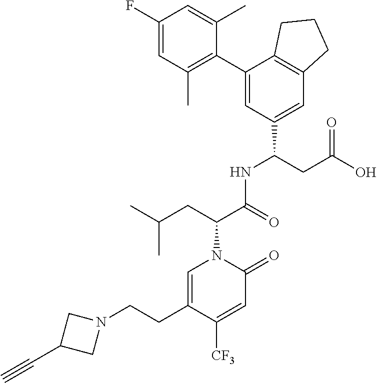 [SMILES: C#CC1CN(CCc2cn([C@H](CC(C)C)C(=O)N[C@@H](CC(=O)O)c3cc4c(c(-c5c(C)cc(F)cc5C)c3)CCC4)c(=O)cc2C(F)(F)F)C1] |
| | 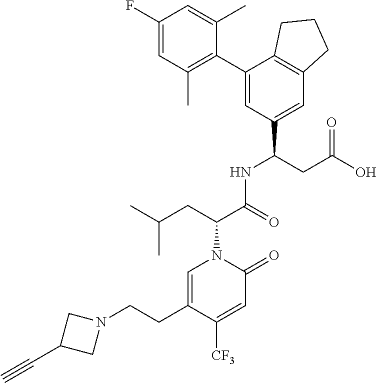 [SMILES: C#CC1CN(CCc2cn([C@H](CC(C)C)C(=O)N[C@H](CC(=O)O)c3cc4c(c(-c5c(C)cc(F)cc5C)c3)CCC4)c(=O)cc2C(F)(F)F)C1] |
| 129 | 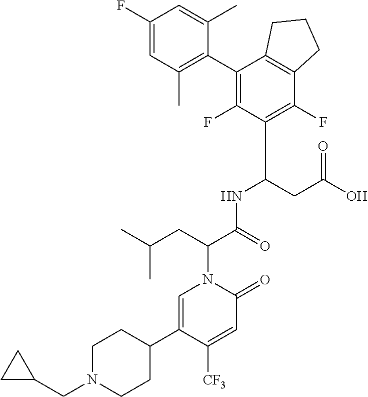 [SMILES: Cc1cc(F)cc(C)c1-c1c(F)c(C(CC(=O)O)NC(=O)C(CC(C)C)n2cc(C3CCN(CC4CC4)CC3)c(C(F)(F)F)cc2=O)c(F)c2c1CCC2] |
| | 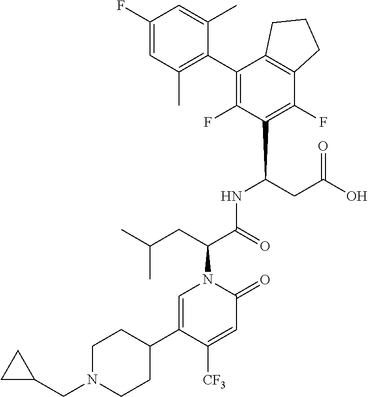 [SMILES: Cc1cc(F)cc(C)c1-c1c(F)c([C@@H](CC(=O)O)NC(=O)[C@H](CC(C)C)n2cc(C3CCN(CC4CC4)CC3)c(C(F)(F)F)cc2=O)c(F)c2c1CCC2] |
| | 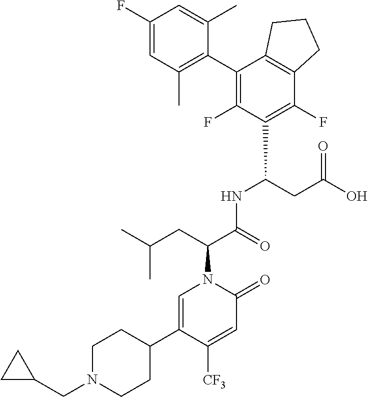 [SMILES: Cc1cc(F)cc(C)c1-c1c(F)c([C@H](CC(=O)O)NC(=O)[C@H](CC(C)C)n2cc(C3CCN(CC4CC4)CC3)c(C(F)(F)F)cc2=O)c(F)c2c1CCC2] |
| | 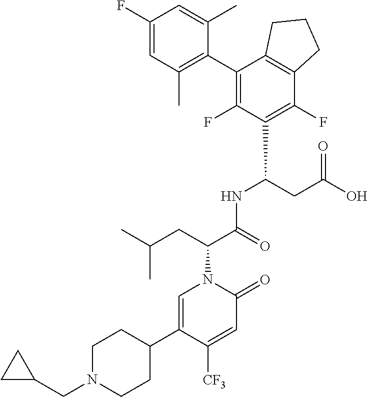 [SMILES: Cc1cc(F)cc(C)c1-c1c(F)c([C@H](CC(=O)O)NC(=O)[C@@H](CC(C)C)n2cc(C3CCN(CC4CC4)CC3)c(C(F)(F)F)cc2=O)c(F)c2c1CCC2] |
| | 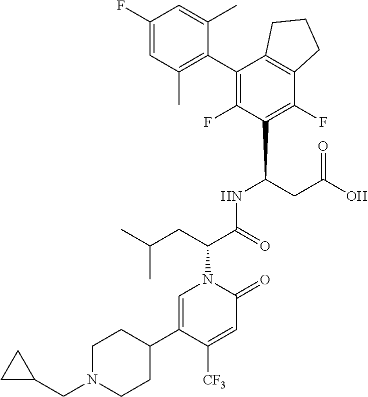 [SMILES: Cc1cc(F)cc(C)c1-c1c(F)c([C@@H](CC(=O)O)NC(=O)[C@@H](CC(C)C)n2cc(C3CCN(CC4CC4)CC3)c(C(F)(F)F)cc2=O)c(F)c2c1CCC2] |
| 130 | 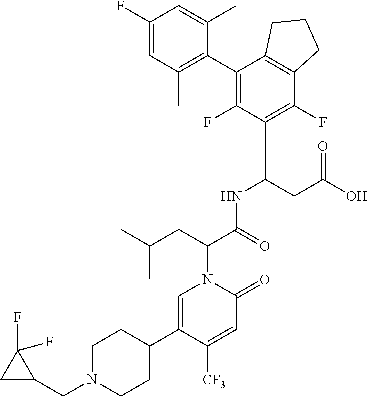 [SMILES: Cc1cc(F)cc(C)c1-c1c(F)c(C(CC(=O)O)NC(=O)C(CC(C)C)n2cc(C3CCN(CC4CC4(F)F)CC3)c(C(F)(F)F)cc2=O)c(F)c2c1CCC2] |
| | 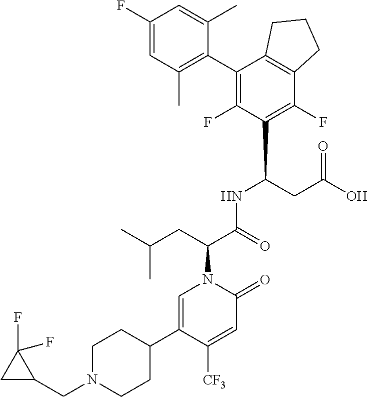 [SMILES: Cc1cc(F)cc(C)c1-c1c(F)c([C@@H](CC(=O)O)NC(=O)[C@H](CC(C)C)n2cc(C3CCN(CC4CC4(F)F)CC3)c(C(F)(F)F)cc2=O)c(F)c2c1CCC2] |
| | 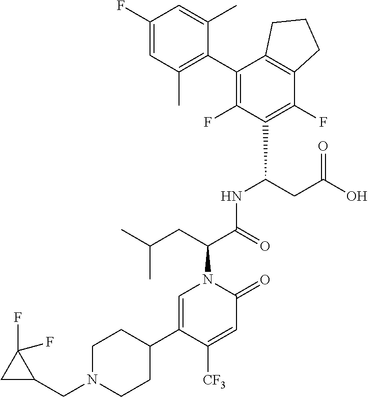 [SMILES: Cc1cc(F)cc(C)c1-c1c(F)c([C@H](CC(=O)O)NC(=O)[C@H](CC(C)C)n2cc(C3CCN(CC4CC4(F)F)CC3)c(C(F)(F)F)cc2=O)c(F)c2c1CCC2] |
| | 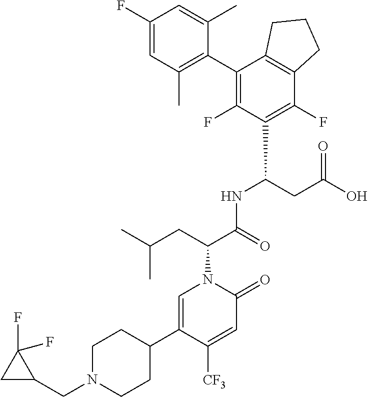 [SMILES: Cc1cc(F)cc(C)c1-c1c(F)c([C@H](CC(=O)O)NC(=O)[C@@H](CC(C)C)n2cc(C3CCN(CC4CC4(F)F)CC3)c(C(F)(F)F)cc2=O)c(F)c2c1CCC2] |
| | 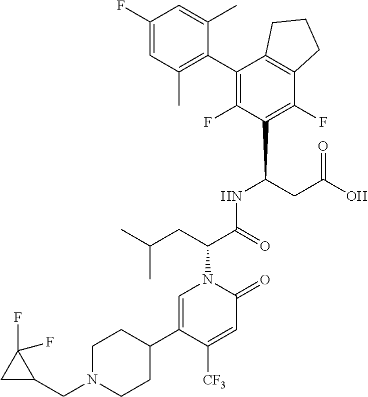 [SMILES: Cc1cc(F)cc(C)c1-c1c(F)c([C@@H](CC(=O)O)NC(=O)[C@@H](CC(C)C)n2cc(C3CCN(CC4CC4(F)F)CC3)c(C(F)(F)F)cc2=O)c(F)c2c1CCC2] |
| 131 | 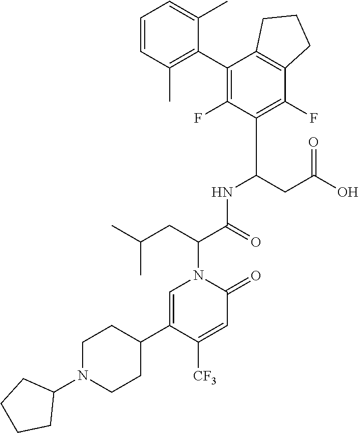 [SMILES: Cc1cccc(C)c1-c1c(F)c(C(CC(=O)O)NC(=O)C(CC(C)C)n2cc(C3CCN(C4CCCC4)CC3)c(C(F)(F)F)cc2=O)c(F)c2c1CCC2] |
| | 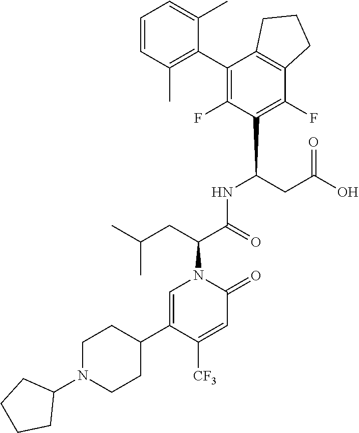 [SMILES: Cc1cccc(C)c1-c1c(F)c([C@@H](CC(=O)O)NC(=O)[C@H](CC(C)C)n2cc(C3CCN(C4CCCC4)CC3)c(C(F)(F)F)cc2=O)c(F)c2c1CCC2] |
| | 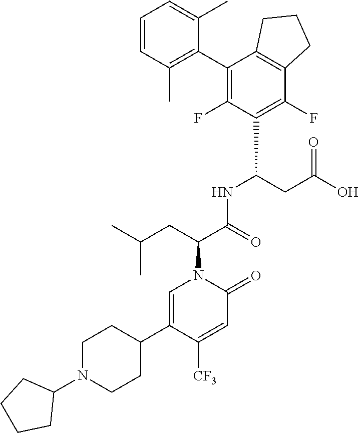 [SMILES: Cc1cccc(C)c1-c1c(F)c([C@H](CC(=O)O)NC(=O)[C@H](CC(C)C)n2cc(C3CCN(C4CCCC4)CC3)c(C(F)(F)F)cc2=O)c(F)c2c1CCC2] |
| | 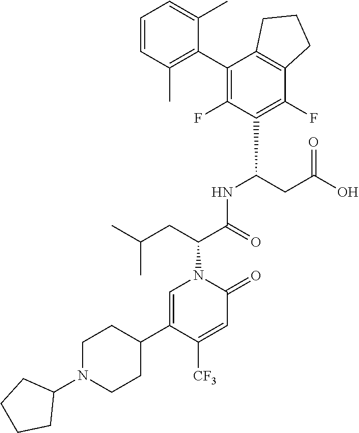 [SMILES: Cc1cccc(C)c1-c1c(F)c([C@H](CC(=O)O)NC(=O)[C@@H](CC(C)C)n2cc(C3CCN(C4CCCC4)CC3)c(C(F)(F)F)cc2=O)c(F)c2c1CCC2] |
| | 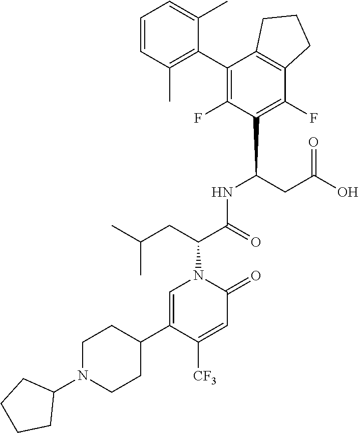 [SMILES: Cc1cccc(C)c1-c1c(F)c([C@@H](CC(=O)O)NC(=O)[C@@H](CC(C)C)n2cc(C3CCN(C4CCCC4)CC3)c(C(F)(F)F)cc2=O)c(F)c2c1CCC2] |
| 132 | 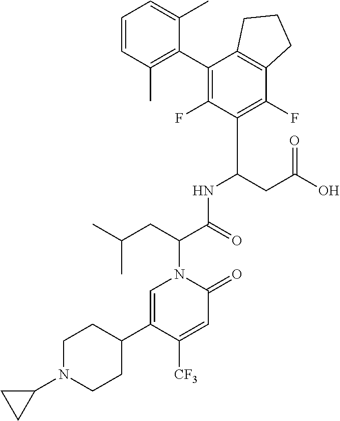 [SMILES: Cc1cccc(C)c1-c1c(F)c(C(CC(=O)O)NC(=O)C(CC(C)C)n2cc(C3CCN(C4CC4)CC3)c(C(F)(F)F)cc2=O)c(F)c2c1CCC2] |
| | 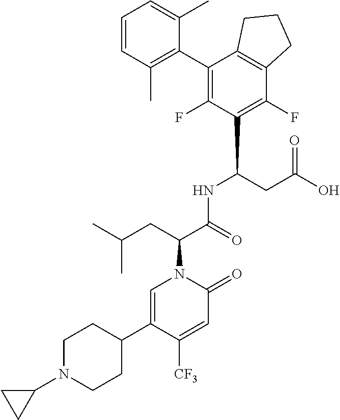 [SMILES: Cc1cccc(C)c1-c1c(F)c([C@@H](CC(=O)O)NC(=O)[C@H](CC(C)C)n2cc(C3CCN(C4CC4)CC3)c(C(F)(F)F)cc2=O)c(F)c2c1CCC2] |
| | 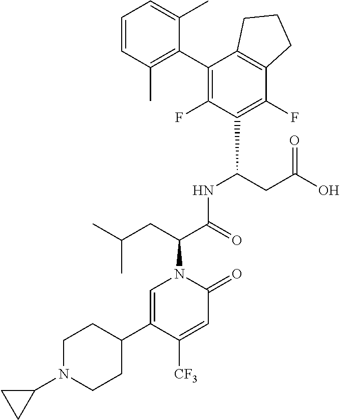 [SMILES: Cc1cccc(C)c1-c1c(F)c([C@H](CC(=O)O)NC(=O)[C@H](CC(C)C)n2cc(C3CCN(C4CC4)CC3)c(C(F)(F)F)cc2=O)c(F)c2c1CCC2] |
| | 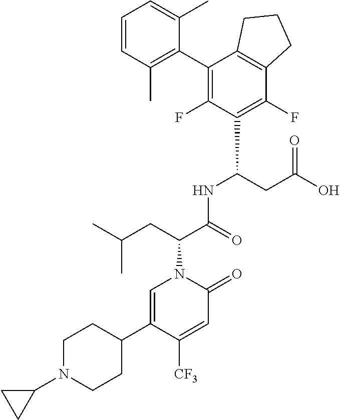 [SMILES: Cc1cccc(C)c1-c1c(F)c([C@H](CC(=O)O)NC(=O)[C@@H](CC(C)C)n2cc(C3CCN(C4CC4)CC3)c(C(F)(F)F)cc2=O)c(F)c2c1CCC2] |
| | 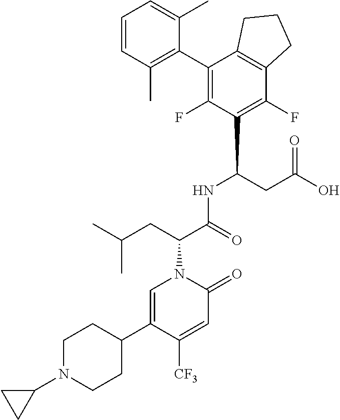 [SMILES: Cc1cccc(C)c1-c1c(F)c([C@@H](CC(=O)O)NC(=O)[C@@H](CC(C)C)n2cc(C3CCN(C4CC4)CC3)c(C(F)(F)F)cc2=O)c(F)c2c1CCC2] |
| 133 | 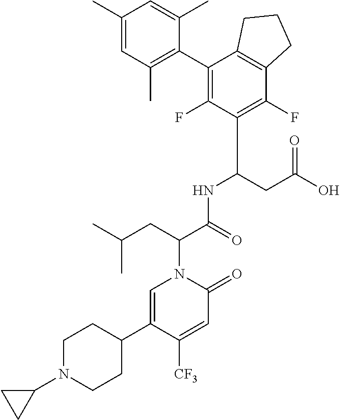 [SMILES: Cc1cc(C)c(-c2c(F)c(C(CC(=O)O)NC(=O)C(CC(C)C)n3cc(C4CCN(C5CC5)CC4)c(C(F)(F)F)cc3=O)c(F)c3c2CCC3)c(C)c1] |
| | 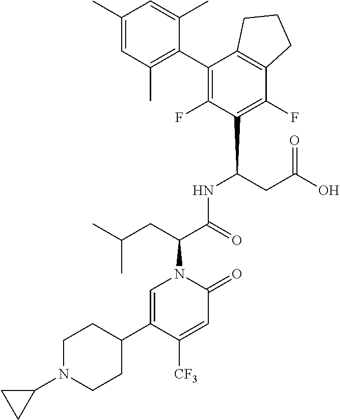 [SMILES: Cc1cc(C)c(-c2c(F)c([C@@H](CC(=O)O)NC(=O)[C@H](CC(C)C)n3cc(C4CCN(C5CC5)CC4)c(C(F)(F)F)cc3=O)c(F)c3c2CCC3)c(C)c1] |
| | 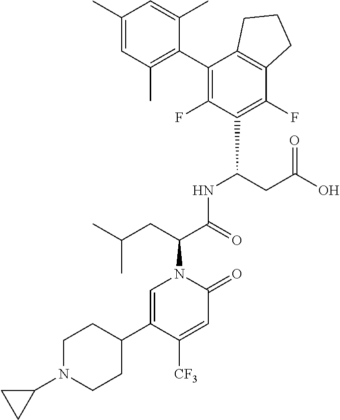 [SMILES: Cc1cc(C)c(-c2c(F)c([C@H](CC(=O)O)NC(=O)[C@H](CC(C)C)n3cc(C4CCN(C5CC5)CC4)c(C(F)(F)F)cc3=O)c(F)c3c2CCC3)c(C)c1] |
| | 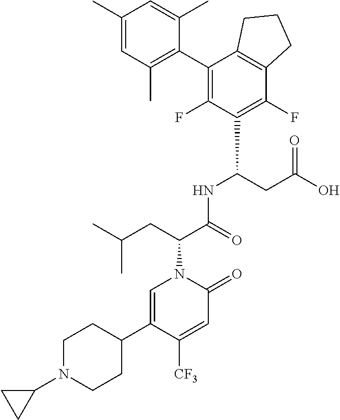 [SMILES: Cc1cc(C)c(-c2c(F)c([C@H](CC(=O)O)NC(=O)[C@@H](CC(C)C)n3cc(C4CCN(C5CC5)CC4)c(C(F)(F)F)cc3=O)c(F)c3c2CCC3)c(C)c1] |
| | 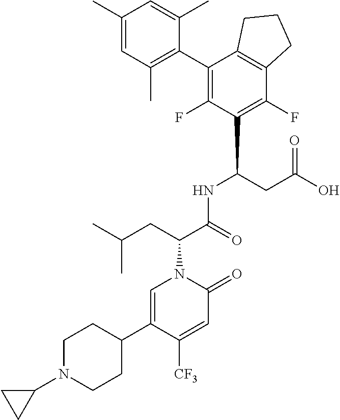 [SMILES: Cc1cc(C)c(-c2c(F)c([C@@H](CC(=O)O)NC(=O)[C@@H](CC(C)C)n3cc(C4CCN(C5CC5)CC4)c(C(F)(F)F)cc3=O)c(F)c3c2CCC3)c(C)c1] |
| 134 | 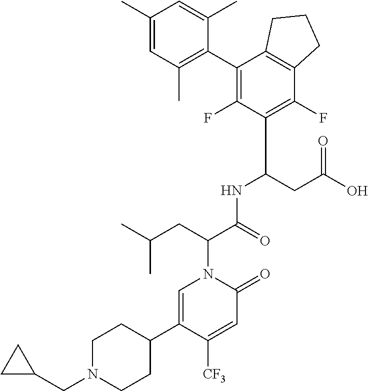 [SMILES: Cc1cc(C)c(-c2c(F)c(C(CC(=O)O)NC(=O)C(CC(C)C)n3cc(C4CCN(CC5CC5)CC4)c(C(F)(F)F)cc3=O)c(F)c3c2CCC3)c(C)c1] |
| | 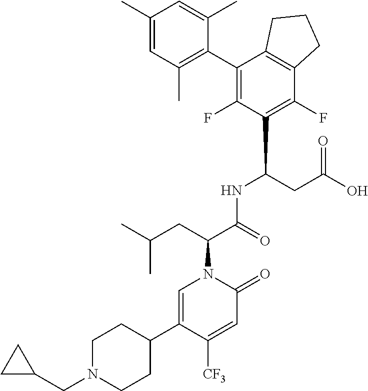 [SMILES: Cc1cc(C)c(-c2c(F)c([C@@H](CC(=O)O)NC(=O)[C@H](CC(C)C)n3cc(C4CCN(CC5CC5)CC4)c(C(F)(F)F)cc3=O)c(F)c3c2CCC3)c(C)c1] |
| | 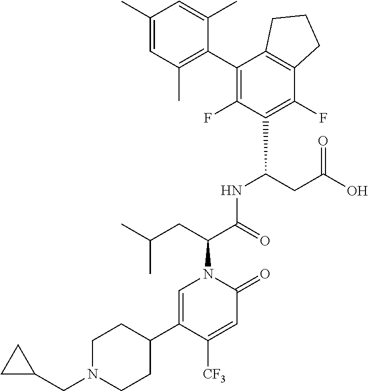 [SMILES: Cc1cc(C)c(-c2c(F)c([C@H](CC(=O)O)NC(=O)[C@H](CC(C)C)n3cc(C4CCN(CC5CC5)CC4)c(C(F)(F)F)cc3=O)c(F)c3c2CCC3)c(C)c1] |
| | 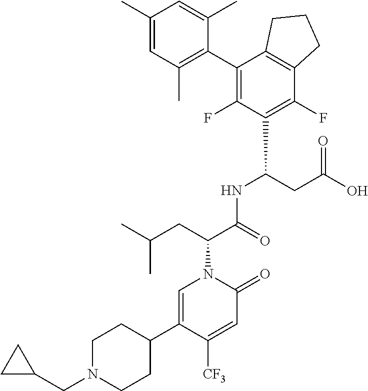 [SMILES: Cc1cc(C)c(-c2c(F)c([C@H](CC(=O)O)NC(=O)[C@@H](CC(C)C)n3cc(C4CCN(CC5CC5)CC4)c(C(F)(F)F)cc3=O)c(F)c3c2CCC3)c(C)c1] |
| | 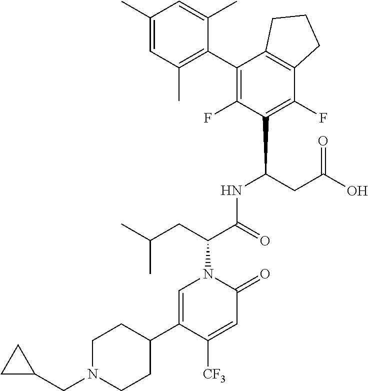 [SMILES: Cc1cc(C)c(-c2c(F)c([C@@H](CC(=O)O)NC(=O)[C@@H](CC(C)C)n3cc(C4CCN(CC5CC5)CC4)c(C(F)(F)F)cc3=O)c(F)c3c2CCC3)c(C)c1] |
| 135 | 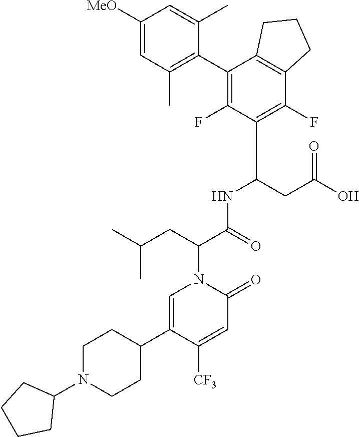 [SMILES: COc1cc(C)c(-c2c(F)c(C(CC(=O)O)NC(=O)C(CC(C)C)n3cc(C4CCN(C5CCCC5)CC4)c(C(F)(F)F)cc3=O)c(F)c3c2CCC3)c(C)c1] |
| | 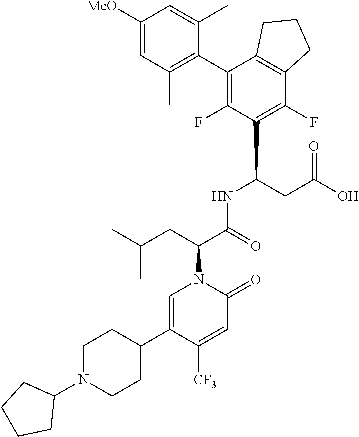 [SMILES: COc1cc(C)c(-c2c(F)c([C@@H](CC(=O)O)NC(=O)[C@H](CC(C)C)n3cc(C4CCN(C5CCCC5)CC4)c(C(F)(F)F)cc3=O)c(F)c3c2CCC3)c(C)c1] |
| | 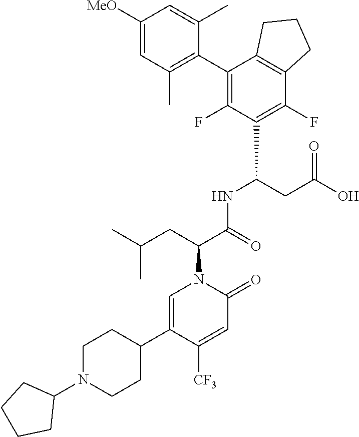 [SMILES: COc1cc(C)c(-c2c(F)c([C@H](CC(=O)O)NC(=O)[C@H](CC(C)C)n3cc(C4CCN(C5CCCC5)CC4)c(C(F)(F)F)cc3=O)c(F)c3c2CCC3)c(C)c1] |
| | 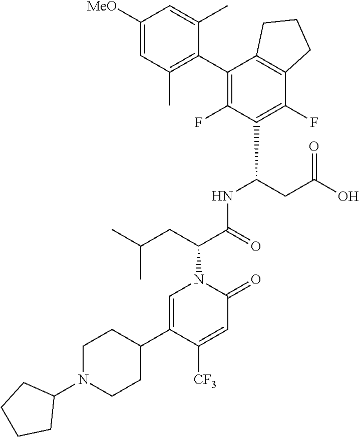 [SMILES: COc1cc(C)c(-c2c(F)c([C@H](CC(=O)O)NC(=O)[C@@H](CC(C)C)n3cc(C4CCN(C5CCCC5)CC4)c(C(F)(F)F)cc3=O)c(F)c3c2CCC3)c(C)c1] |
| | 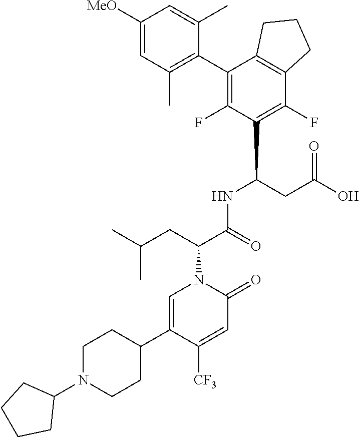 [SMILES: COc1cc(C)c(-c2c(F)c([C@@H](CC(=O)O)NC(=O)[C@@H](CC(C)C)n3cc(C4CCN(C5CCCC5)CC4)c(C(F)(F)F)cc3=O)c(F)c3c2CCC3)c(C)c1] |
| 136 | 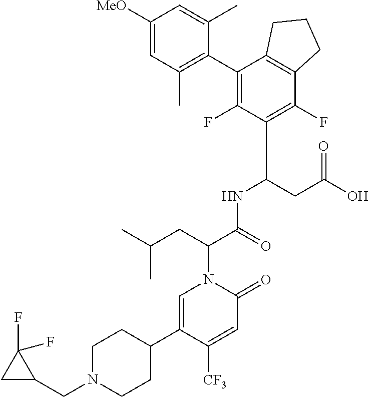 [SMILES: COc1cc(C)c(-c2c(F)c(C(CC(=O)O)NC(=O)C(CC(C)C)n3cc(C4CCN(CC5CC5(F)F)CC4)c(C(F)(F)F)cc3=O)c(F)c3c2CCC3)c(C)c1] |
| | 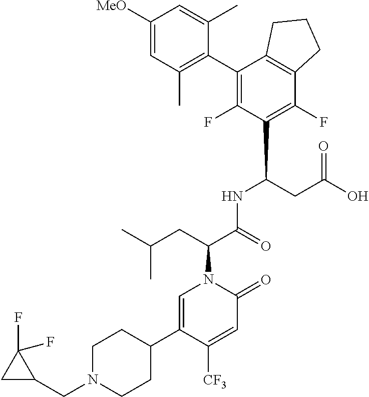 [SMILES: COc1cc(C)c(-c2c(F)c([C@@H](CC(=O)O)NC(=O)[C@H](CC(C)C)n3cc(C4CCN(CC5CC5(F)F)CC4)c(C(F)(F)F)cc3=O)c(F)c3c2CCC3)c(C)c1] |
| | 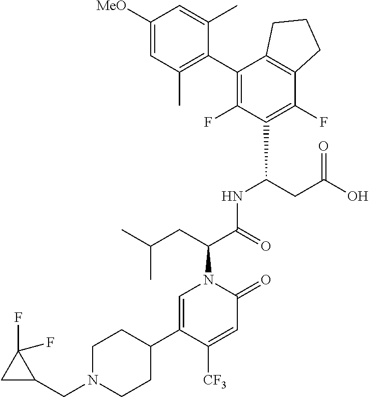 [SMILES: COc1cc(C)c(-c2c(F)c([C@H](CC(=O)O)NC(=O)[C@H](CC(C)C)n3cc(C4CCN(CC5CC5(F)F)CC4)c(C(F)(F)F)cc3=O)c(F)c3c2CCC3)c(C)c1] |
| | 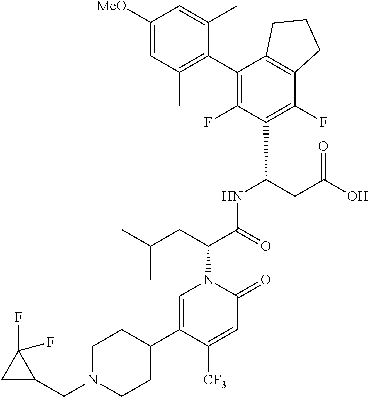 [SMILES: COc1cc(C)c(-c2c(F)c([C@H](CC(=O)O)NC(=O)[C@@H](CC(C)C)n3cc(C4CCN(CC5CC5(F)F)CC4)c(C(F)(F)F)cc3=O)c(F)c3c2CCC3)c(C)c1] |
| | 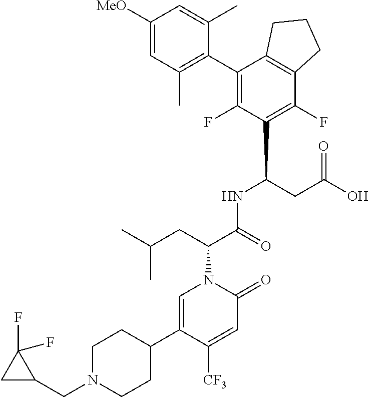 [SMILES: COc1cc(C)c(-c2c(F)c([C@@H](CC(=O)O)NC(=O)[C@@H](CC(C)C)n3cc(C4CCN(CC5CC5(F)F)CC4)c(C(F)(F)F)cc3=O)c(F)c3c2CCC3)c(C)c1] |
| 137 | 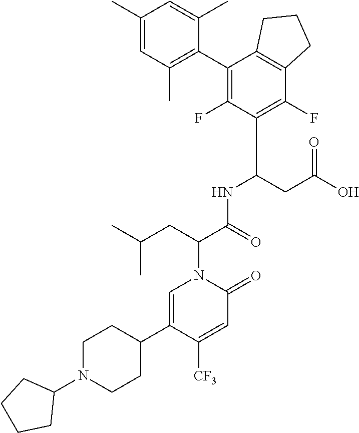 [SMILES: Cc1cc(C)c(-c2c(F)c(C(CC(=O)O)NC(=O)C(CC(C)C)n3cc(C4CCN(C5CCCC5)CC4)c(C(F)(F)F)cc3=O)c(F)c3c2CCC3)c(C)c1] |
| | 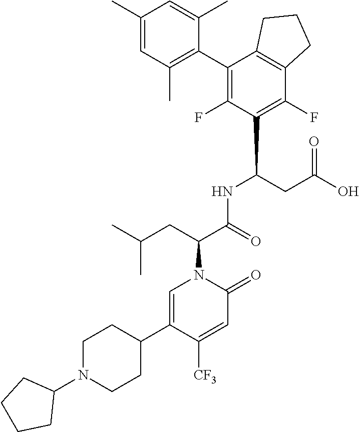 [SMILES: Cc1cc(C)c(-c2c(F)c([C@@H](CC(=O)O)NC(=O)[C@H](CC(C)C)n3cc(C4CCN(C5CCCC5)CC4)c(C(F)(F)F)cc3=O)c(F)c3c2CCC3)c(C)c1] |
| | 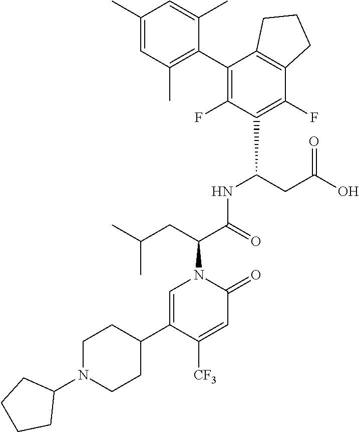 [SMILES: Cc1cc(C)c(-c2c(F)c([C@H](CC(=O)O)NC(=O)[C@H](CC(C)C)n3cc(C4CCN(C5CCCC5)CC4)c(C(F)(F)F)cc3=O)c(F)c3c2CCC3)c(C)c1] |
| | 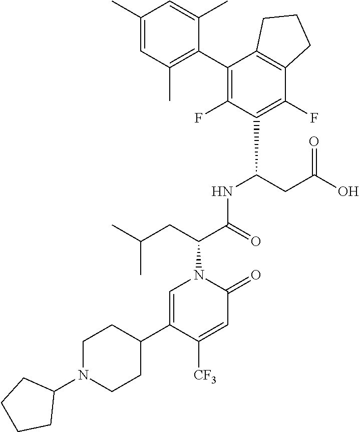 [SMILES: Cc1cc(C)c(-c2c(F)c([C@H](CC(=O)O)NC(=O)[C@@H](CC(C)C)n3cc(C4CCN(C5CCCC5)CC4)c(C(F)(F)F)cc3=O)c(F)c3c2CCC3)c(C)c1] |
| | 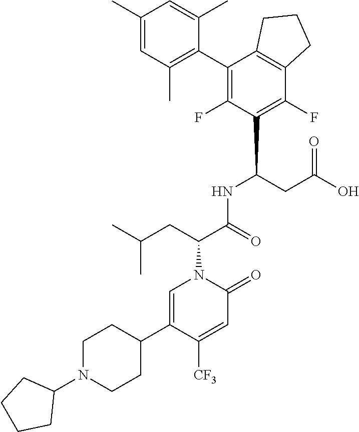 [SMILES: Cc1cc(C)c(-c2c(F)c([C@@H](CC(=O)O)NC(=O)[C@@H](CC(C)C)n3cc(C4CCN(C5CCCC5)CC4)c(C(F)(F)F)cc3=O)c(F)c3c2CCC3)c(C)c1] |
| 138 | 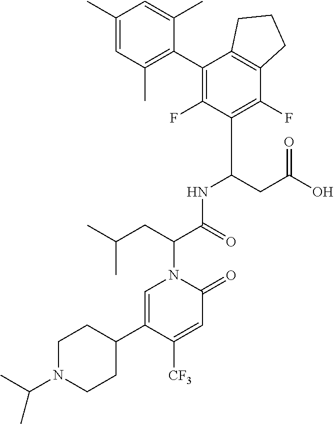 [SMILES: Cc1cc(C)c(-c2c(F)c(C(CC(=O)O)NC(=O)C(CC(C)C)n3cc(C4CCN(C(C)C)CC4)c(C(F)(F)F)cc3=O)c(F)c3c2CCC3)c(C)c1] |
| | 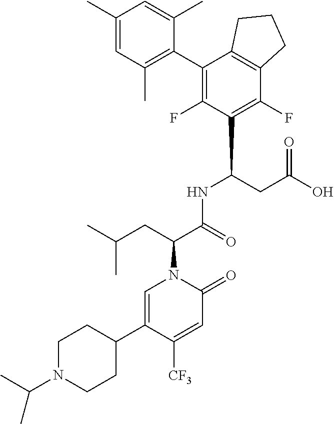 [SMILES: Cc1cc(C)c(-c2c(F)c([C@@H](CC(=O)O)NC(=O)[C@H](CC(C)C)n3cc(C4CCN(C(C)C)CC4)c(C(F)(F)F)cc3=O)c(F)c3c2CCC3)c(C)c1] |
| | 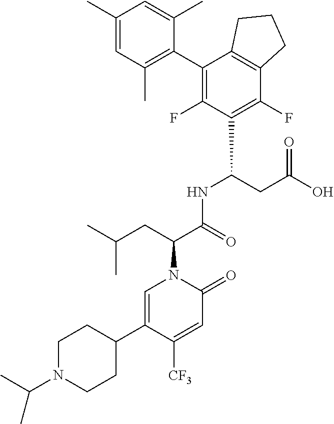 [SMILES: Cc1cc(C)c(-c2c(F)c([C@H](CC(=O)O)NC(=O)[C@H](CC(C)C)n3cc(C4CCN(C(C)C)CC4)c(C(F)(F)F)cc3=O)c(F)c3c2CCC3)c(C)c1] |
| | 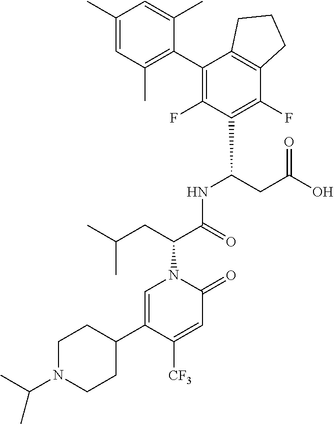 [SMILES: Cc1cc(C)c(-c2c(F)c([C@H](CC(=O)O)NC(=O)[C@@H](CC(C)C)n3cc(C4CCN(C(C)C)CC4)c(C(F)(F)F)cc3=O)c(F)c3c2CCC3)c(C)c1] |
| | 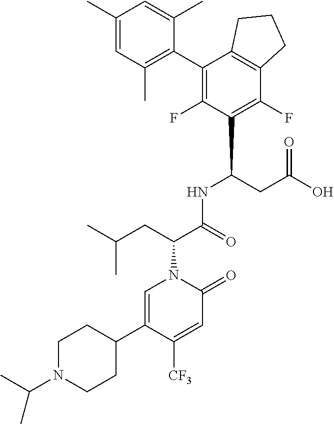 [SMILES: Cc1cc(C)c(-c2c(F)c([C@@H](CC(=O)O)NC(=O)[C@@H](CC(C)C)n3cc(C4CCN(C(C)C)CC4)c(C(F)(F)F)cc3=O)c(F)c3c2CCC3)c(C)c1] |
| 139 | 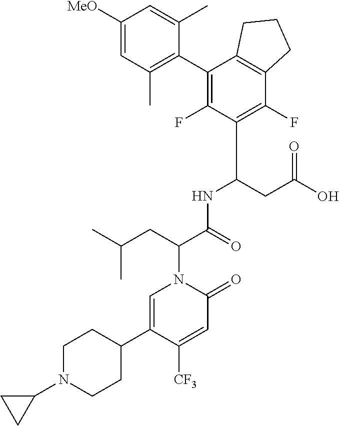 [SMILES: COc1cc(C)c(-c2c(F)c(C(CC(=O)O)NC(=O)C(CC(C)C)n3cc(C4CCN(C5CC5)CC4)c(C(F)(F)F)cc3=O)c(F)c3c2CCC3)c(C)c1] |
| | 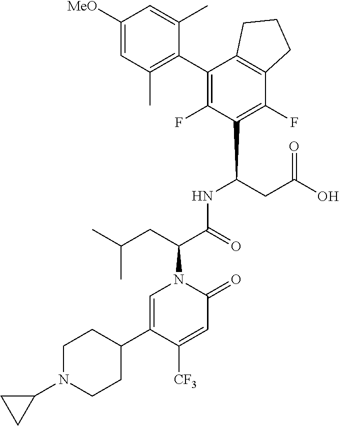 [SMILES: COc1cc(C)c(-c2c(F)c([C@@H](CC(=O)O)NC(=O)[C@H](CC(C)C)n3cc(C4CCN(C5CC5)CC4)c(C(F)(F)F)cc3=O)c(F)c3c2CCC3)c(C)c1] |
| | 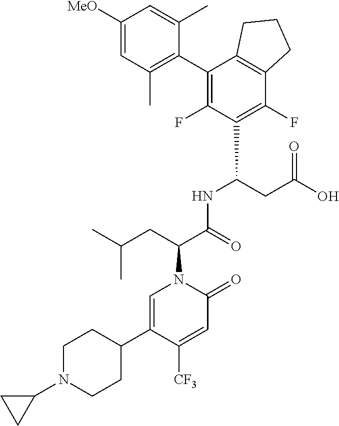 [SMILES: COc1cc(C)c(-c2c(F)c([C@H](CC(=O)O)NC(=O)[C@H](CC(C)C)n3cc(C4CCN(C5CC5)CC4)c(C(F)(F)F)cc3=O)c(F)c3c2CCC3)c(C)c1] |
| | 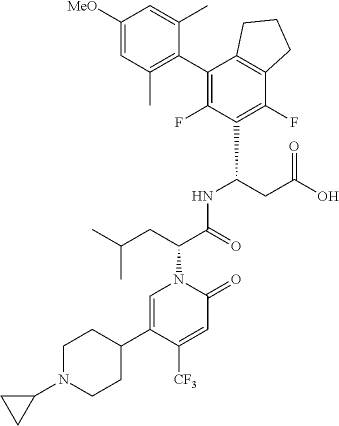 [SMILES: COc1cc(C)c(-c2c(F)c([C@H](CC(=O)O)NC(=O)[C@@H](CC(C)C)n3cc(C4CCN(C5CC5)CC4)c(C(F)(F)F)cc3=O)c(F)c3c2CCC3)c(C)c1] |
| | 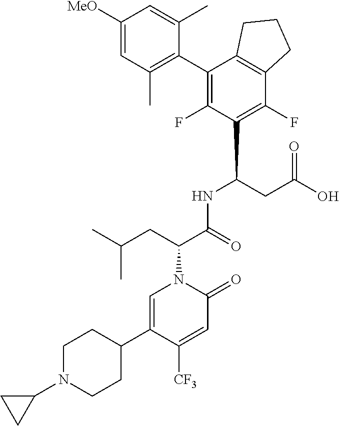 [SMILES: COc1cc(C)c(-c2c(F)c([C@@H](CC(=O)O)NC(=O)[C@@H](CC(C)C)n3cc(C4CCN(C5CC5)CC4)c(C(F)(F)F)cc3=O)c(F)c3c2CCC3)c(C)c1] |
| 140 | 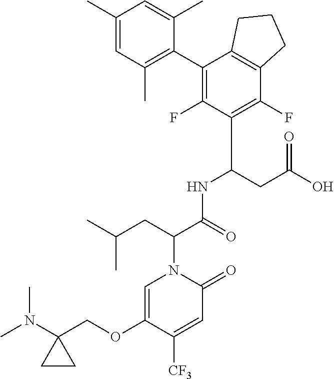 [SMILES: Cc1cc(C)c(-c2c(F)c(C(CC(=O)O)NC(=O)C(CC(C)C)n3cc(OCC4(N(C)C)CC4)c(C(F)(F)F)cc3=O)c(F)c3c2CCC3)c(C)c1] |
| | 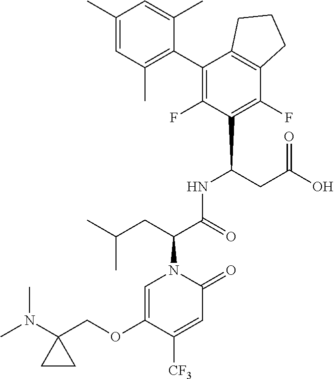 [SMILES: Cc1cc(C)c(-c2c(F)c([C@@H](CC(=O)O)NC(=O)[C@H](CC(C)C)n3cc(OCC4(N(C)C)CC4)c(C(F)(F)F)cc3=O)c(F)c3c2CCC3)c(C)c1] |
| | 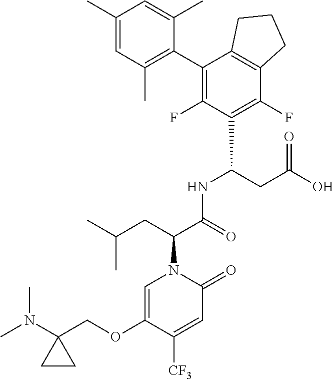 [SMILES: Cc1cc(C)c(-c2c(F)c([C@H](CC(=O)O)NC(=O)[C@H](CC(C)C)n3cc(OCC4(N(C)C)CC4)c(C(F)(F)F)cc3=O)c(F)c3c2CCC3)c(C)c1] |
| | 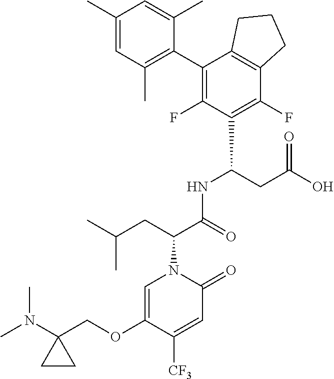 [SMILES: Cc1cc(C)c(-c2c(F)c([C@H](CC(=O)O)NC(=O)[C@@H](CC(C)C)n3cc(OCC4(N(C)C)CC4)c(C(F)(F)F)cc3=O)c(F)c3c2CCC3)c(C)c1] |
| | 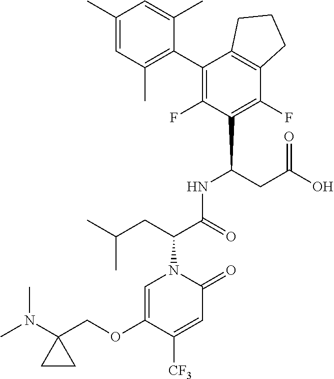 [SMILES: Cc1cc(C)c(-c2c(F)c([C@@H](CC(=O)O)NC(=O)[C@@H](CC(C)C)n3cc(OCC4(N(C)C)CC4)c(C(F)(F)F)cc3=O)c(F)c3c2CCC3)c(C)c1] |
| 141 | 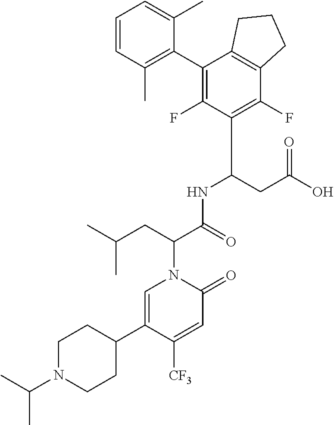 [SMILES: Cc1cccc(C)c1-c1c(F)c(C(CC(=O)O)NC(=O)C(CC(C)C)n2cc(C3CCN(C(C)C)CC3)c(C(F)(F)F)cc2=O)c(F)c2c1CCC2] |
| | 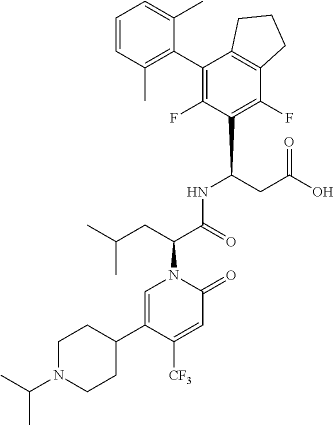 [SMILES: Cc1cccc(C)c1-c1c(F)c([C@@H](CC(=O)O)NC(=O)[C@H](CC(C)C)n2cc(C3CCN(C(C)C)CC3)c(C(F)(F)F)cc2=O)c(F)c2c1CCC2] |
| | 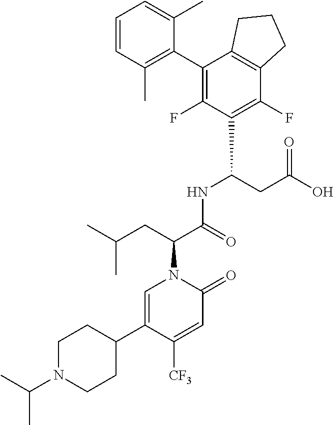 [SMILES: Cc1cccc(C)c1-c1c(F)c([C@H](CC(=O)O)NC(=O)[C@H](CC(C)C)n2cc(C3CCN(C(C)C)CC3)c(C(F)(F)F)cc2=O)c(F)c2c1CCC2] |
| | 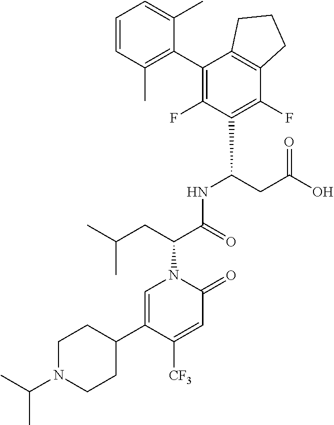 [SMILES: Cc1cccc(C)c1-c1c(F)c([C@H](CC(=O)O)NC(=O)[C@@H](CC(C)C)n2cc(C3CCN(C(C)C)CC3)c(C(F)(F)F)cc2=O)c(F)c2c1CCC2] |
| | 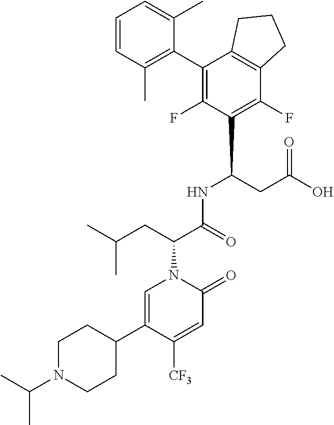 [SMILES: Cc1cccc(C)c1-c1c(F)c([C@@H](CC(=O)O)NC(=O)[C@@H](CC(C)C)n2cc(C3CCN(C(C)C)CC3)c(C(F)(F)F)cc2=O)c(F)c2c1CCC2] |
| 142 | 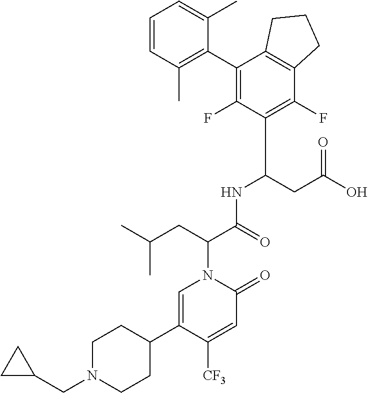 [SMILES: Cc1cccc(C)c1-c1c(F)c(C(CC(=O)O)NC(=O)C(CC(C)C)n2cc(C3CCN(CC4CC4)CC3)c(C(F)(F)F)cc2=O)c(F)c2c1CCC2] |
| | 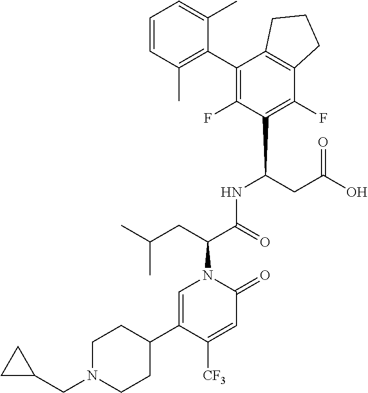 [SMILES: Cc1cccc(C)c1-c1c(F)c([C@@H](CC(=O)O)NC(=O)[C@H](CC(C)C)n2cc(C3CCN(CC4CC4)CC3)c(C(F)(F)F)cc2=O)c(F)c2c1CCC2] |
| | 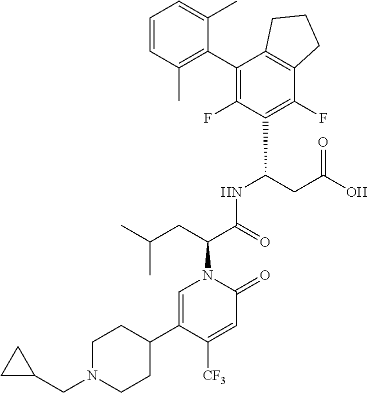 [SMILES: Cc1cccc(C)c1-c1c(F)c([C@H](CC(=O)O)NC(=O)[C@H](CC(C)C)n2cc(C3CCN(CC4CC4)CC3)c(C(F)(F)F)cc2=O)c(F)c2c1CCC2] |
| | 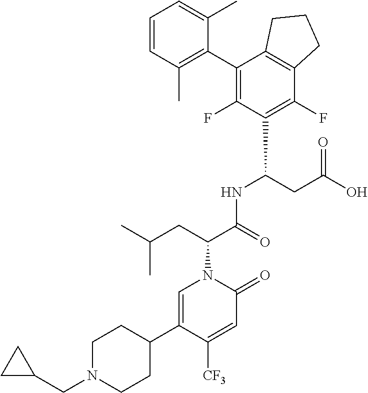 [SMILES: Cc1cccc(C)c1-c1c(F)c([C@H](CC(=O)O)NC(=O)[C@@H](CC(C)C)n2cc(C3CCN(CC4CC4)CC3)c(C(F)(F)F)cc2=O)c(F)c2c1CCC2] |
| | 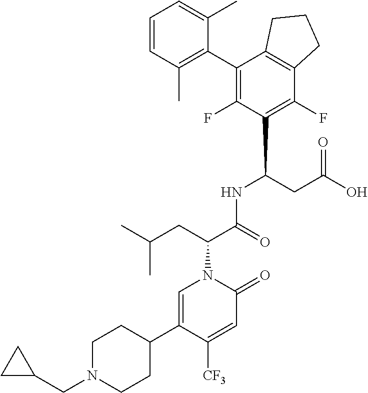 [SMILES: Cc1cccc(C)c1-c1c(F)c([C@@H](CC(=O)O)NC(=O)[C@@H](CC(C)C)n2cc(C3CCN(CC4CC4)CC3)c(C(F)(F)F)cc2=O)c(F)c2c1CCC2] |
| 143 | 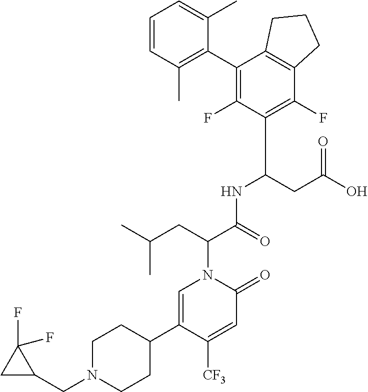 [SMILES: Cc1cccc(C)c1-c1c(F)c(C(CC(=O)O)NC(=O)C(CC(C)C)n2cc(C3CCN(CC4CC4(F)F)CC3)c(C(F)(F)F)cc2=O)c(F)c2c1CCC2] |
| | 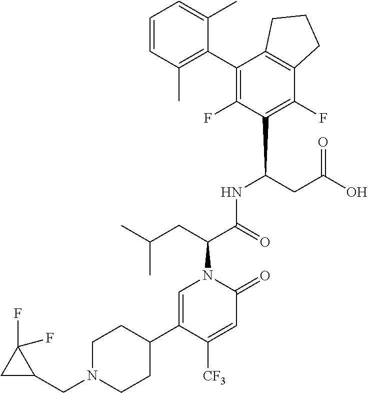 [SMILES: Cc1cccc(C)c1-c1c(F)c([C@@H](CC(=O)O)NC(=O)[C@H](CC(C)C)n2cc(C3CCN(CC4CC4(F)F)CC3)c(C(F)(F)F)cc2=O)c(F)c2c1CCC2] |
| | 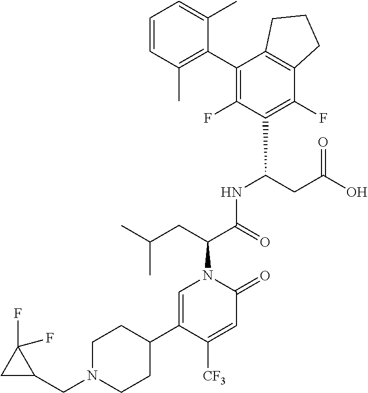 [SMILES: Cc1cccc(C)c1-c1c(F)c([C@H](CC(=O)O)NC(=O)[C@H](CC(C)C)n2cc(C3CCN(CC4CC4(F)F)CC3)c(C(F)(F)F)cc2=O)c(F)c2c1CCC2] |
| | 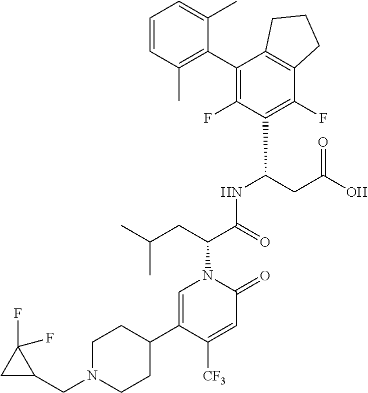 [SMILES: Cc1cccc(C)c1-c1c(F)c([C@H](CC(=O)O)NC(=O)[C@@H](CC(C)C)n2cc(C3CCN(CC4CC4(F)F)CC3)c(C(F)(F)F)cc2=O)c(F)c2c1CCC2] |
| | 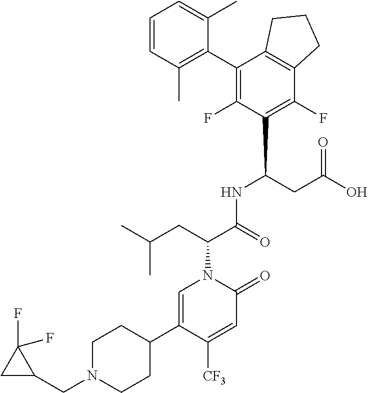 [SMILES: Cc1cccc(C)c1-c1c(F)c([C@@H](CC(=O)O)NC(=O)[C@@H](CC(C)C)n2cc(C3CCN(CC4CC4(F)F)CC3)c(C(F)(F)F)cc2=O)c(F)c2c1CCC2] |
| 144 | 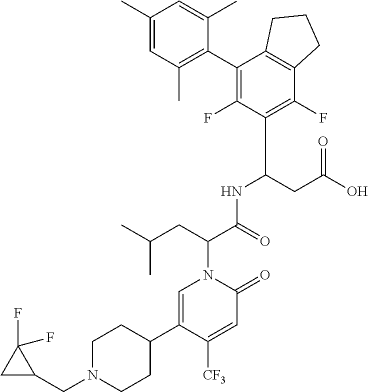 [SMILES: Cc1cc(C)c(-c2c(F)c(C(CC(=O)O)NC(=O)C(CC(C)C)n3cc(C4CCN(CC5CC5(F)F)CC4)c(C(F)(F)F)cc3=O)c(F)c3c2CCC3)c(C)c1] |
| | 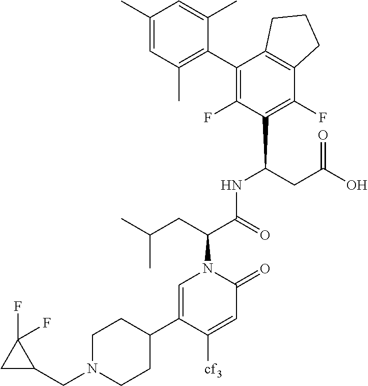 |
| | 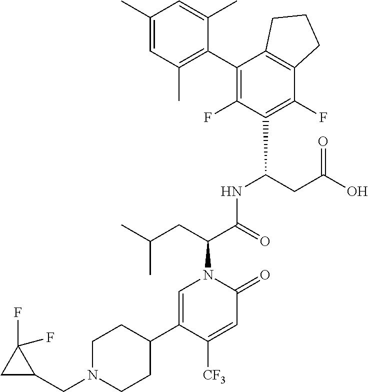 [SMILES: Cc1cc(C)c(-c2c(F)c([C@H](CC(=O)O)NC(=O)[C@H](CC(C)C)n3cc(C4CCN(CC5CC5(F)F)CC4)c(C(F)(F)F)cc3=O)c(F)c3c2CCC3)c(C)c1] |
| | 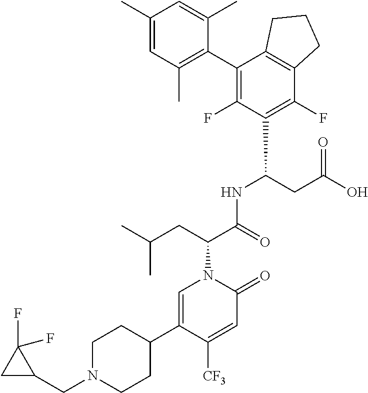 [SMILES: Cc1cc(C)c(-c2c(F)c([C@H](CC(=O)O)NC(=O)[C@@H](CC(C)C)n3cc(C4CCN(CC5CC5(F)F)CC4)c(C(F)(F)F)cc3=O)c(F)c3c2CCC3)c(C)c1] |
| | 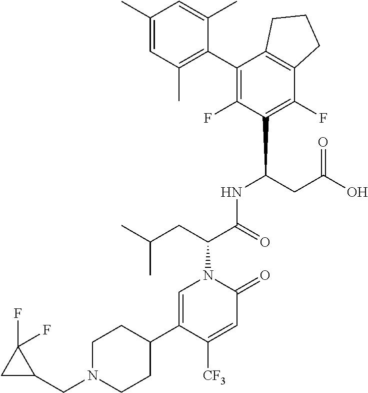 [SMILES: Cc1cc(C)c(-c2c(F)c([C@@H](CC(=O)O)NC(=O)[C@@H](CC(C)C)n3cc(C4CCN(CC5CC5(F)F)CC4)c(C(F)(F)F)cc3=O)c(F)c3c2CCC3)c(C)c1] |
| 145 | 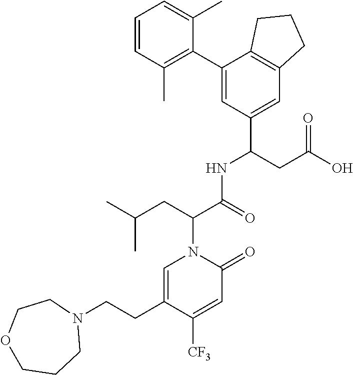 [SMILES: Cc1cccc(C)c1-c1cc(C(CC(=O)O)NC(=O)C(CC(C)C)n2cc(CCN3CCCOCC3)c(C(F)(F)F)cc2=O)cc2c1CCC2] |
| | 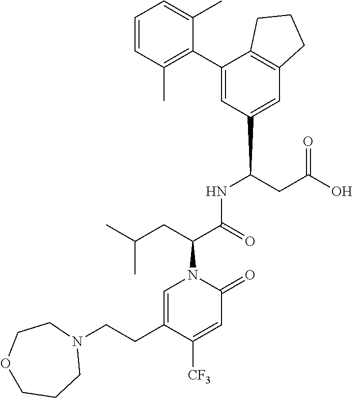 [SMILES: Cc1cccc(C)c1-c1cc([C@@H](CC(=O)O)NC(=O)[C@H](CC(C)C)n2cc(CCN3CCCOCC3)c(C(F)(F)F)cc2=O)cc2c1CCC2] |
| | 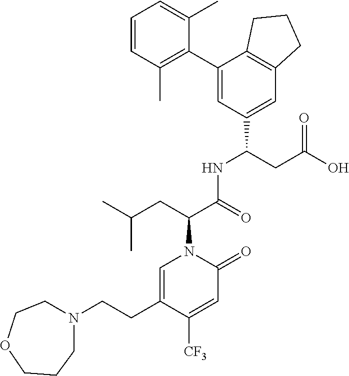 [SMILES: Cc1cccc(C)c1-c1cc([C@H](CC(=O)O)NC(=O)[C@H](CC(C)C)n2cc(CCN3CCCOCC3)c(C(F)(F)F)cc2=O)cc2c1CCC2] |
| | 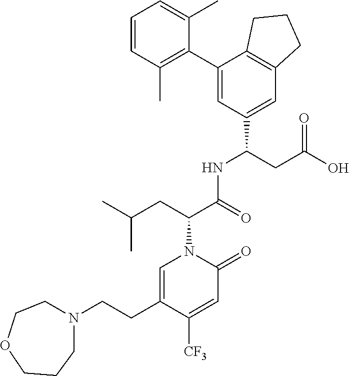 [SMILES: Cc1cccc(C)c1-c1cc([C@H](CC(=O)O)NC(=O)[C@@H](CC(C)C)n2cc(CCN3CCCOCC3)c(C(F)(F)F)cc2=O)cc2c1CCC2] |
| | 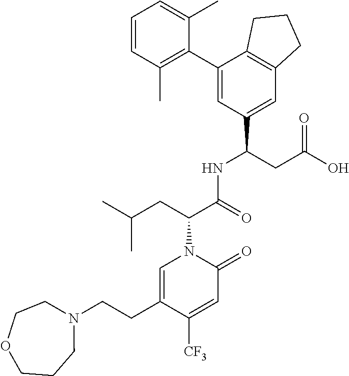 [SMILES: Cc1cccc(C)c1-c1cc([C@@H](CC(=O)O)NC(=O)[C@@H](CC(C)C)n2cc(CCN3CCCOCC3)c(C(F)(F)F)cc2=O)cc2c1CCC2] |
| 146 | 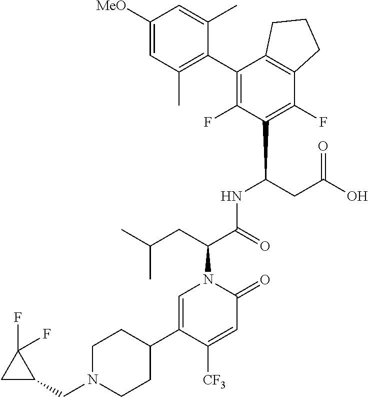 [SMILES: COc1cc(C)c(-c2c(F)c([C@@H](CC(=O)O)NC(=O)[C@H](CC(C)C)n3cc(C4CCN(C[C@@H]5CC5(F)F)CC4)c(C(F)(F)F)cc3=O)c(F)c3c2CCC3)c(C)c1] |
| | 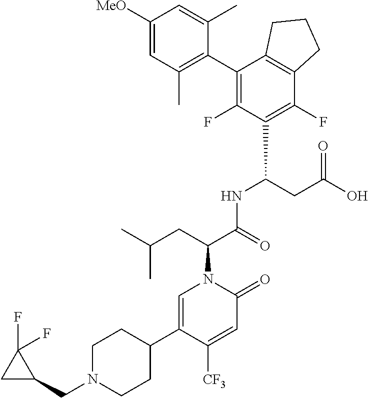 [SMILES: COc1cc(C)c(-c2c(F)c([C@H](CC(=O)O)NC(=O)[C@H](CC(C)C)n3cc(C4CCN(C[C@H]5CC5(F)F)CC4)c(C(F)(F)F)cc3=O)c(F)c3c2CCC3)c(C)c1] |
| | 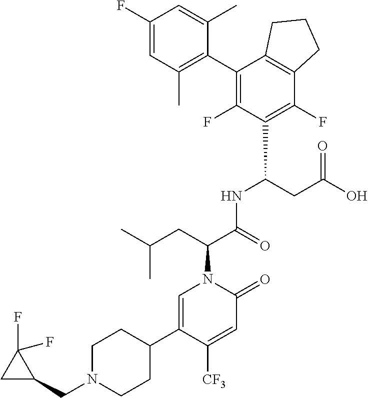 [SMILES: Cc1cc(F)cc(C)c1-c1c(F)c([C@H](CC(=O)O)NC(=O)[C@H](CC(C)C)n2cc(C3CCN(C[C@H]4CC4(F)F)CC3)c(C(F)(F)F)cc2=O)c(F)c2c1CCC2] |
| | 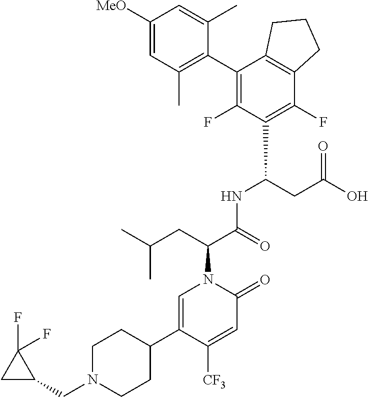 [SMILES: COc1cc(C)c(-c2c(F)c([C@H](CC(=O)O)NC(=O)[C@H](CC(C)C)n3cc(C4CCN(C[C@@H]5CC5(F)F)CC4)c(C(F)(F)F)cc3=O)c(F)c3c2CCC3)c(C)c1] |
| | 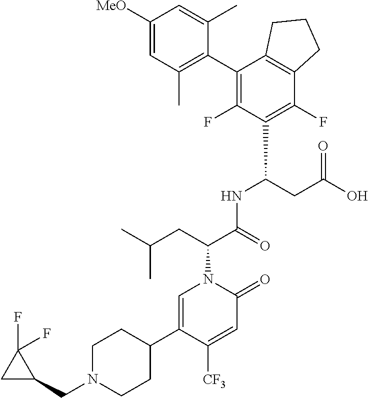 [SMILES: COc1cc(C)c(-c2c(F)c([C@H](CC(=O)O)NC(=O)[C@@H](CC(C)C)n3cc(C4CCN(C[C@H]5CC5(F)F)CC4)c(C(F)(F)F)cc3=O)c(F)c3c2CCC3)c(C)c1] |
| 147 | 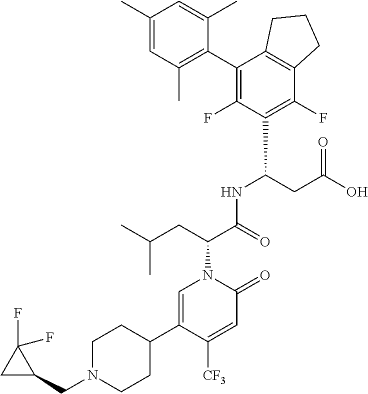 [SMILES: Cc1cc(C)c(-c2c(F)c([C@H](CC(=O)O)NC(=O)[C@@H](CC(C)C)n3cc(C4CCN(C[C@H]5CC5(F)F)CC4)c(C(F)(F)F)cc3=O)c(F)c3c2CCC3)c(C)c1] |
| | 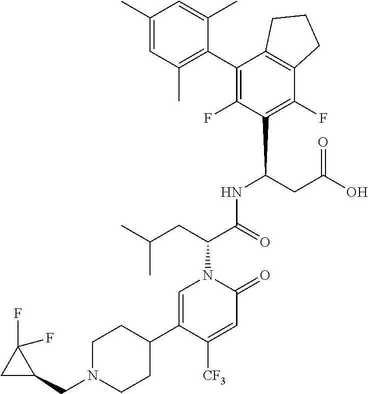 [SMILES: Cc1cc(C)c(-c2c(F)c([C@@H](CC(=O)O)NC(=O)[C@@H](CC(C)C)n3cc(C4CCN(C[C@H]5CC5(F)F)CC4)c(C(F)(F)F)cc3=O)c(F)c3c2CCC3)c(C)c1] |
| | 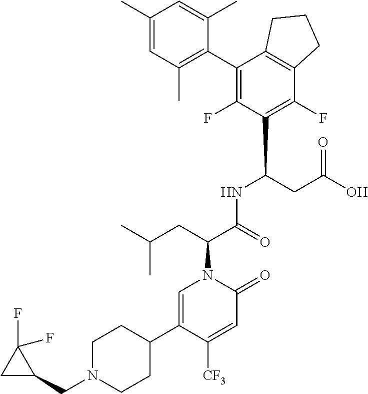 [SMILES: Cc1cc(C)c(-c2c(F)c([C@@H](CC(=O)O)NC(=O)[C@H](CC(C)C)n3cc(C4CCN(C[C@H]5CC5(F)F)CC4)c(C(F)(F)F)cc3=O)c(F)c3c2CCC3)c(C)c1] |
| | 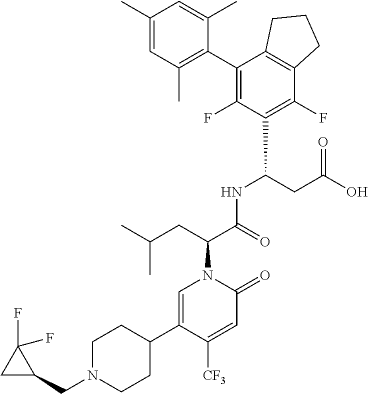 [SMILES: Cc1cc(C)c(-c2c(F)c([C@H](CC(=O)O)NC(=O)[C@H](CC(C)C)n3cc(C4CCN(C[C@H]5CC5(F)F)CC4)c(C(F)(F)F)cc3=O)c(F)c3c2CCC3)c(C)c1] |
| | 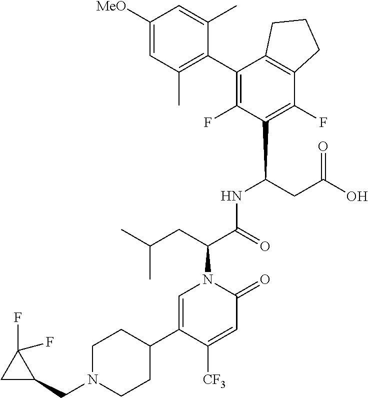 [SMILES: COc1cc(C)c(-c2c(F)c([C@@H](CC(=O)O)NC(=O)[C@H](CC(C)C)n3cc(C4CCN(C[C@H]5CC5(F)F)CC4)c(C(F)(F)F)cc3=O)c(F)c3c2CCC3)c(C)c1] |
| 148 | 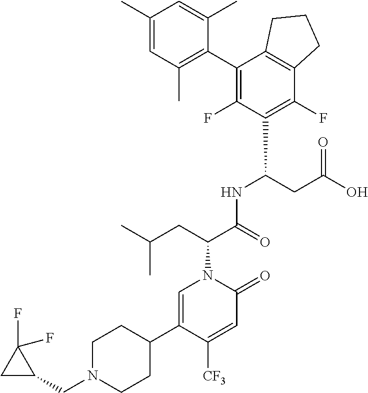 [SMILES: Cc1cc(C)c(-c2c(F)c([C@H](CC(=O)O)NC(=O)[C@@H](CC(C)C)n3cc(C4CCN(C[C@@H]5CC5(F)F)CC4)c(C(F)(F)F)cc3=O)c(F)c3c2CCC3)c(C)c1] |
| | 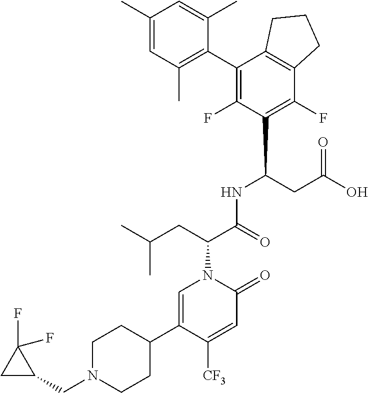 [SMILES: Cc1cc(C)c(-c2c(F)c([C@@H](CC(=O)O)NC(=O)[C@@H](CC(C)C)n3cc(C4CCN(C[C@@H]5CC5(F)F)CC4)c(C(F)(F)F)cc3=O)c(F)c3c2CCC3)c(C)c1] |
| | 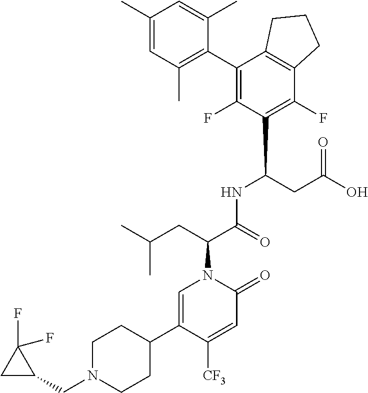 [SMILES: Cc1cc(C)c(-c2c(F)c([C@@H](CC(=O)O)NC(=O)[C@H](CC(C)C)n3cc(C4CCN(C[C@@H]5CC5(F)F)CC4)c(C(F)(F)F)cc3=O)c(F)c3c2CCC3)c(C)c1] |
| | 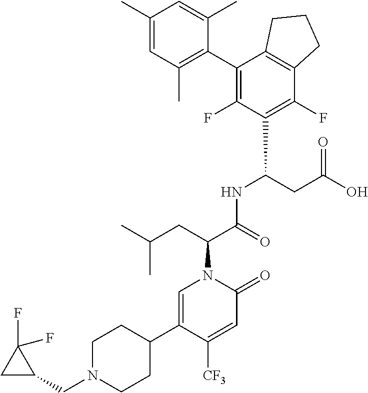 [SMILES: Cc1cc(C)c(-c2c(F)c([C@H](CC(=O)O)NC(=O)[C@H](CC(C)C)n3cc(C4CCN(C[C@@H]5CC5(F)F)CC4)c(C(F)(F)F)cc3=O)c(F)c3c2CCC3)c(C)c1] |
| | 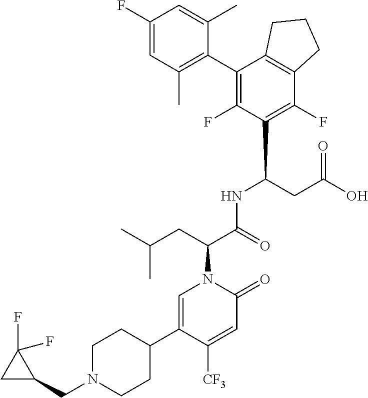 [SMILES: Cc1cc(F)cc(C)c1-c1c(F)c([C@@H](CC(=O)O)NC(=O)[C@H](CC(C)C)n2cc(C3CCN(C[C@H]4CC4(F)F)CC3)c(C(F)(F)F)cc2=O)c(F)c2c1CCC2] |
| 149 | 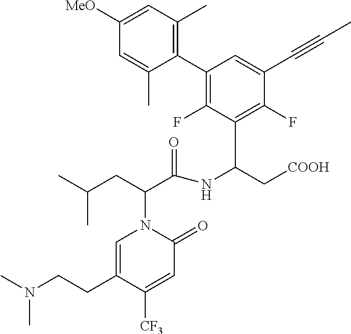 [SMILES: CC#Cc1cc(-c2c(C)cc(OC)cc2C)c(F)c(C(CC(=O)O)NC(=O)C(CC(C)C)n2cc(CCN(C)C)c(C(F)(F)F)cc2=O)c1F] |
| | 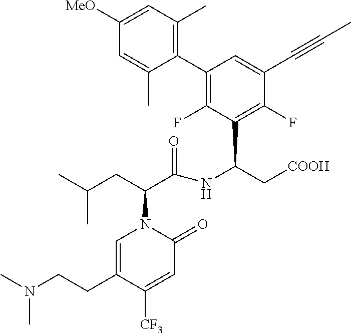 [SMILES: CC#Cc1cc(-c2c(C)cc(OC)cc2C)c(F)c([C@@H](CC(=O)O)NC(=O)[C@H](CC(C)C)n2cc(CCN(C)C)c(C(F)(F)F)cc2=O)c1F] |
| | 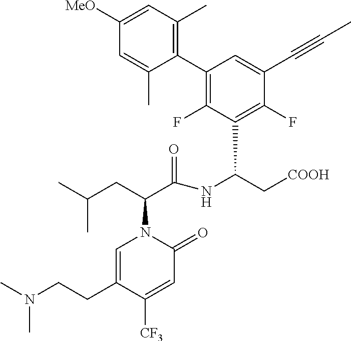 [SMILES: CC#Cc1cc(-c2c(C)cc(OC)cc2C)c(F)c([C@H](CC(=O)O)NC(=O)[C@H](CC(C)C)n2cc(CCN(C)C)c(C(F)(F)F)cc2=O)c1F] |
| | 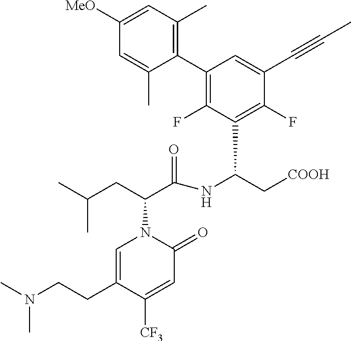 [SMILES: CC#Cc1cc(-c2c(C)cc(OC)cc2C)c(F)c([C@H](CC(=O)O)NC(=O)[C@@H](CC(C)C)n2cc(CCN(C)C)c(C(F)(F)F)cc2=O)c1F] |
| | 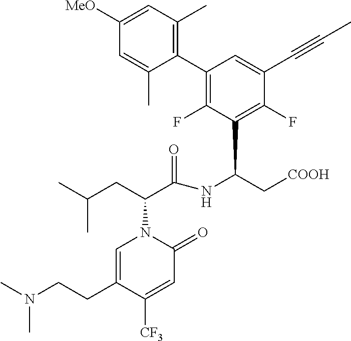 [SMILES: CC#Cc1cc(-c2c(C)cc(OC)cc2C)c(F)c([C@@H](CC(=O)O)NC(=O)[C@@H](CC(C)C)n2cc(CCN(C)C)c(C(F)(F)F)cc2=O)c1F] |
| 150 | 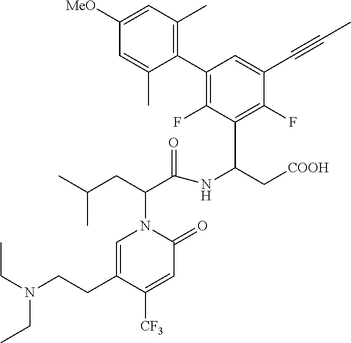 [SMILES: CC#Cc1cc(-c2c(C)cc(OC)cc2C)c(F)c(C(CC(=O)O)NC(=O)C(CC(C)C)n2cc(CCN(CC)CC)c(C(F)(F)F)cc2=O)c1F] |
| | 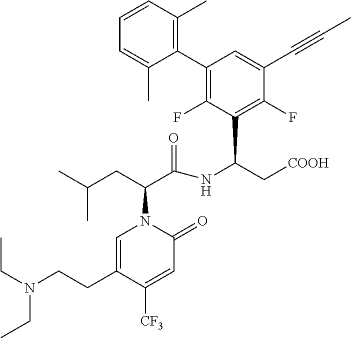 [SMILES: CC#Cc1cc(-c2c(C)cccc2C)c(F)c([C@@H](CC(=O)O)NC(=O)[C@H](CC(C)C)n2cc(CCN(CC)CC)c(C(F)(F)F)cc2=O)c1F] |
| | 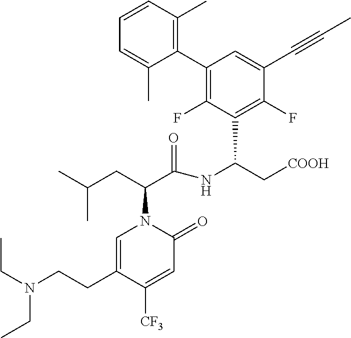 [SMILES: CC#Cc1cc(-c2c(C)cccc2C)c(F)c([C@H](CC(=O)O)NC(=O)[C@H](CC(C)C)n2cc(CCN(CC)CC)c(C(F)(F)F)cc2=O)c1F] |
| | 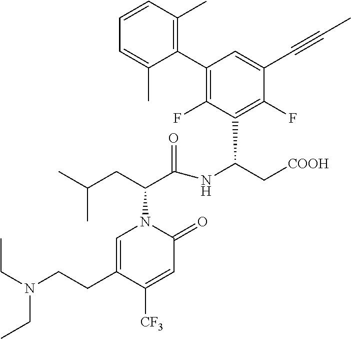 [SMILES: CC#Cc1cc(-c2c(C)cccc2C)c(F)c([C@H](CC(=O)O)NC(=O)[C@@H](CC(C)C)n2cc(CCN(CC)CC)c(C(F)(F)F)cc2=O)c1F] |
| | 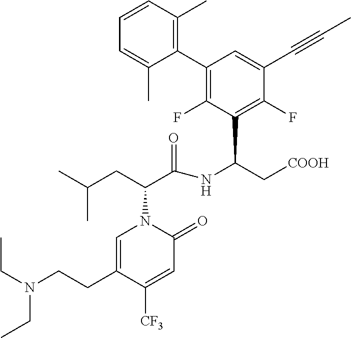 [SMILES: CC#Cc1cc(-c2c(C)cccc2C)c(F)c([C@@H](CC(=O)O)NC(=O)[C@@H](CC(C)C)n2cc(CCN(CC)CC)c(C(F)(F)F)cc2=O)c1F] |
| 151 | 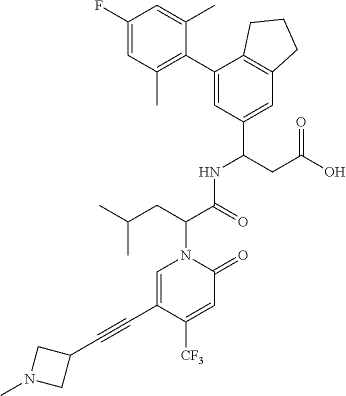 [SMILES: Cc1cc(F)cc(C)c1-c1cc(C(CC(=O)O)NC(=O)C(CC(C)C)n2cc(C#CC3CN(C)C3)c(C(F)(F)F)cc2=O)cc2c1CCC2] |
| | 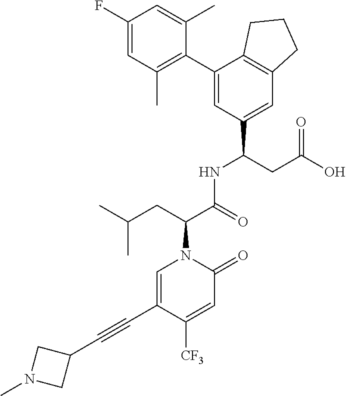 [SMILES: Cc1cc(F)cc(C)c1-c1cc([C@@H](CC(=O)O)NC(=O)[C@H](CC(C)C)n2cc(C#CC3CN(C)C3)c(C(F)(F)F)cc2=O)cc2c1CCC2] |
| | 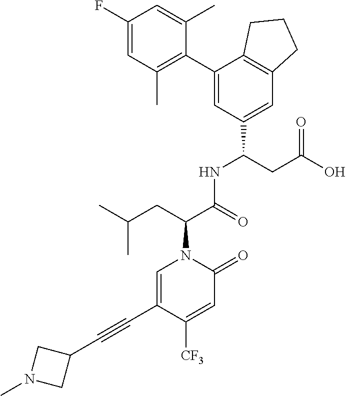 [SMILES: Cc1cc(F)cc(C)c1-c1cc([C@H](CC(=O)O)NC(=O)[C@H](CC(C)C)n2cc(C#CC3CN(C)C3)c(C(F)(F)F)cc2=O)cc2c1CCC2] |
| | 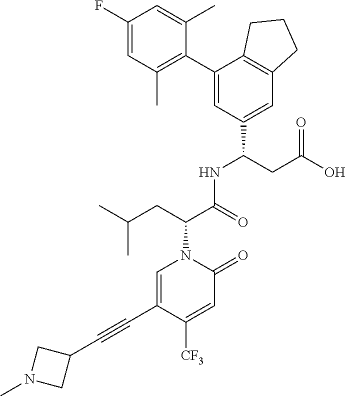 [SMILES: Cc1cc(F)cc(C)c1-c1cc([C@H](CC(=O)O)NC(=O)[C@@H](CC(C)C)n2cc(C#CC3CN(C)C3)c(C(F)(F)F)cc2=O)cc2c1CCC2] |
| | 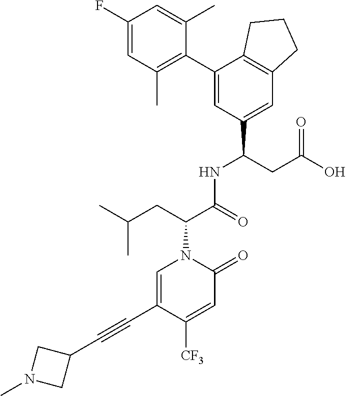 [SMILES: Cc1cc(F)cc(C)c1-c1cc([C@@H](CC(=O)O)NC(=O)[C@@H](CC(C)C)n2cc(C#CC3CN(C)C3)c(C(F)(F)F)cc2=O)cc2c1CCC2] |
| 152 | 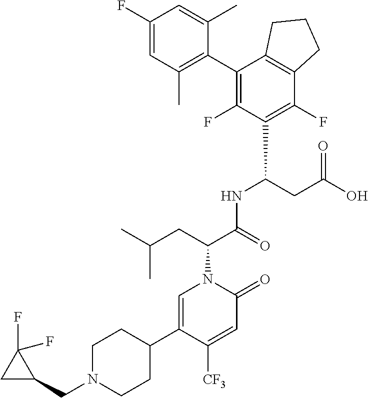 [SMILES: Cc1cc(F)cc(C)c1-c1c(F)c([C@H](CC(=O)O)NC(=O)[C@@H](CC(C)C)n2cc(C3CCN(C[C@H]4CC4(F)F)CC3)c(C(F)(F)F)cc2=O)c(F)c2c1CCC2] |
| | 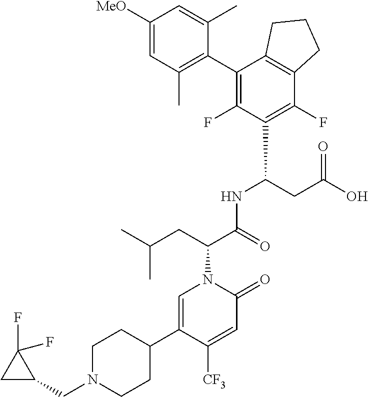 [SMILES: COc1cc(C)c(-c2c(F)c([C@H](CC(=O)O)NC(=O)[C@@H](CC(C)C)n3cc(C4CCN(C[C@@H]5CC5(F)F)CC4)c(C(F)(F)F)cc3=O)c(F)c3c2CCC3)c(C)c1] |
| | 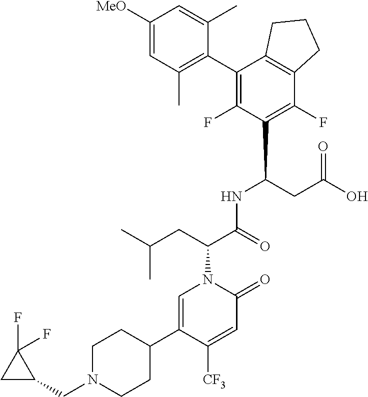 [SMILES: COc1cc(C)c(-c2c(F)c([C@@H](CC(=O)O)NC(=O)[C@@H](CC(C)C)n3cc(C4CCN(C[C@@H]5CC5(F)F)CC4)c(C(F)(F)F)cc3=O)c(F)c3c2CCC3)c(C)c1] |
| | 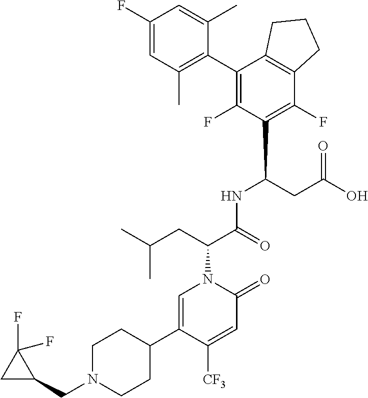 [SMILES: Cc1cc(F)cc(C)c1-c1c(F)c([C@@H](CC(=O)O)NC(=O)[C@@H](CC(C)C)n2cc(C3CCN(C[C@H]4CC4(F)F)CC3)c(C(F)(F)F)cc2=O)c(F)c2c1CCC2] |
| | 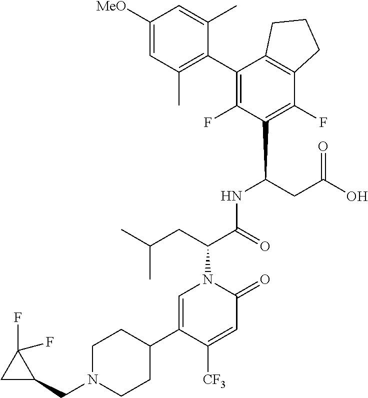 [SMILES: COc1cc(C)c(-c2c(F)c([C@@H](CC(=O)O)NC(=O)[C@@H](CC(C)C)n3cc(C4CCN(C[C@H]5CC5(F)F)CC4)c(C(F)(F)F)cc3=O)c(F)c3c2CCC3)c(C)c1] |
| 153 | 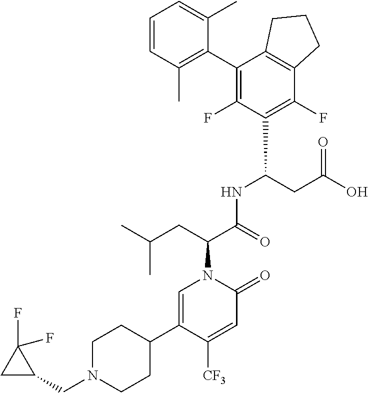 [SMILES: Cc1cccc(C)c1-c1c(F)c([C@H](CC(=O)O)NC(=O)[C@H](CC(C)C)n2cc(C3CCN(C[C@@H]4CC4(F)F)CC3)c(C(F)(F)F)cc2=O)c(F)c2c1CCC2] |
| | 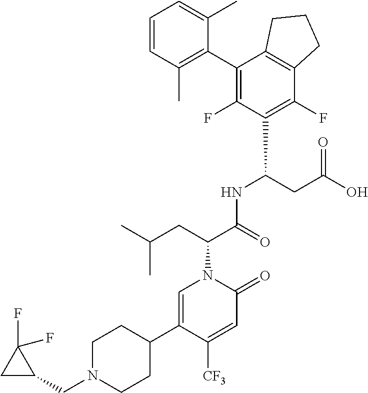 [SMILES: Cc1cccc(C)c1-c1c(F)c([C@H](CC(=O)O)NC(=O)[C@@H](CC(C)C)n2cc(C3CCN(C[C@@H]4CC4(F)F)CC3)c(C(F)(F)F)cc2=O)c(F)c2c1CCC2] |
| | 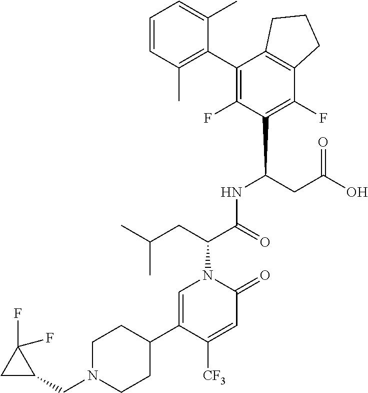 [SMILES: Cc1cccc(C)c1-c1c(F)c([C@@H](CC(=O)O)NC(=O)[C@@H](CC(C)C)n2cc(C3CCN(C[C@@H]4CC4(F)F)CC3)c(C(F)(F)F)cc2=O)c(F)c2c1CCC2] |
| | 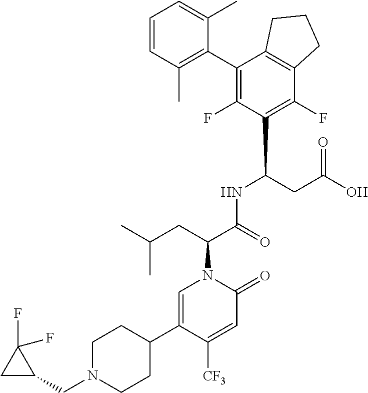 [SMILES: Cc1cccc(C)c1-c1c(F)c([C@@H](CC(=O)O)NC(=O)[C@H](CC(C)C)n2cc(C3CCN(C[C@@H]4CC4(F)F)CC3)c(C(F)(F)F)cc2=O)c(F)c2c1CCC2] |
| | 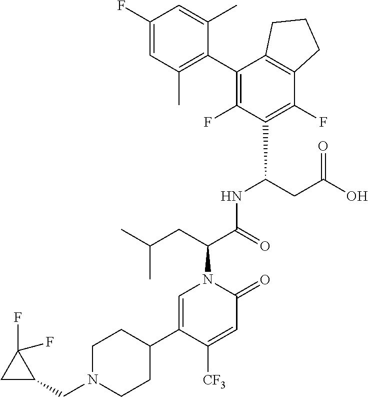 [SMILES: Cc1cc(F)cc(C)c1-c1c(F)c([C@H](CC(=O)O)NC(=O)[C@H](CC(C)C)n2cc(C3CCN(C[C@@H]4CC4(F)F)CC3)c(C(F)(F)F)cc2=O)c(F)c2c1CCC2] |
| 154 | 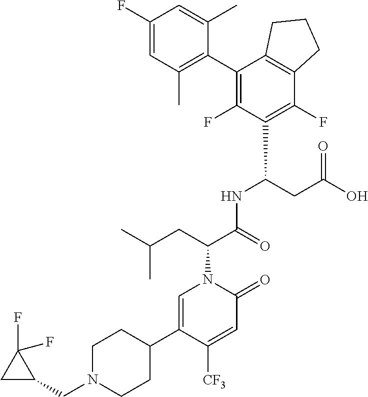 [SMILES: Cc1cc(F)cc(C)c1-c1c(F)c([C@H](CC(=O)O)NC(=O)[C@@H](CC(C)C)n2cc(C3CCN(C[C@@H]4CC4(F)F)CC3)c(C(F)(F)F)cc2=O)c(F)c2c1CCC2] |
| | 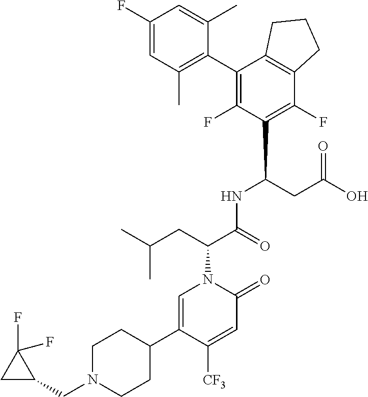 [SMILES: Cc1cc(F)cc(C)c1-c1c(F)c([C@@H](CC(=O)O)NC(=O)[C@@H](CC(C)C)n2cc(C3CCN(C[C@@H]4CC4(F)F)CC3)c(C(F)(F)F)cc2=O)c(F)c2c1CCC2] |
| 155 | 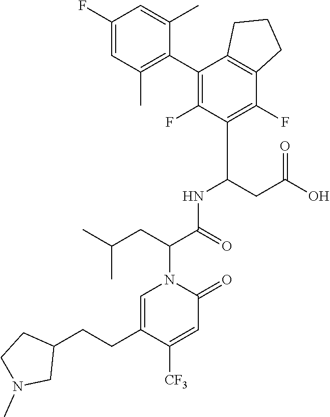 [SMILES: Cc1cc(F)cc(C)c1-c1c(F)c(C(CC(=O)O)NC(=O)C(CC(C)C)n2cc(CCC3CCN(C)C3)c(C(F)(F)F)cc2=O)c(F)c2c1CCC2] |
| | 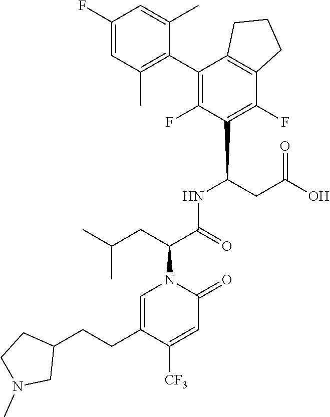 [SMILES: Cc1cc(F)cc(C)c1-c1c(F)c([C@@H](CC(=O)O)NC(=O)[C@H](CC(C)C)n2cc(CCC3CCN(C)C3)c(C(F)(F)F)cc2=O)c(F)c2c1CCC2] |
| | 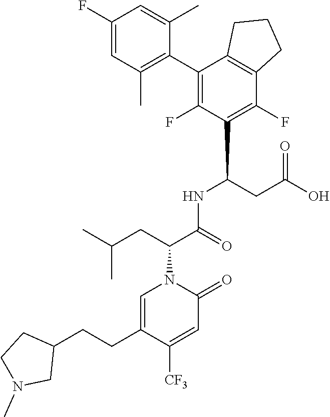 [SMILES: Cc1cc(F)cc(C)c1-c1c(F)c([C@@H](CC(=O)O)NC(=O)[C@@H](CC(C)C)n2cc(CCC3CCN(C)C3)c(C(F)(F)F)cc2=O)c(F)c2c1CCC2] |
| | 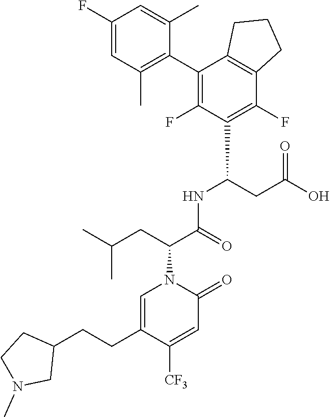 [SMILES: Cc1cc(F)cc(C)c1-c1c(F)c([C@H](CC(=O)O)NC(=O)[C@@H](CC(C)C)n2cc(CCC3CCN(C)C3)c(C(F)(F)F)cc2=O)c(F)c2c1CCC2] |
| | 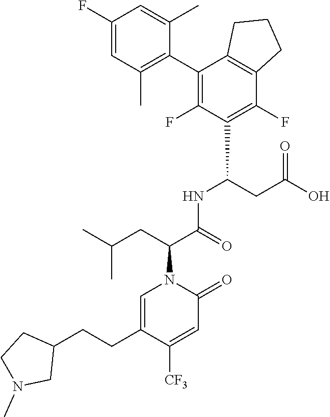 [SMILES: Cc1cc(F)cc(C)c1-c1c(F)c([C@H](CC(=O)O)NC(=O)[C@H](CC(C)C)n2cc(CCC3CCN(C)C3)c(C(F)(F)F)cc2=O)c(F)c2c1CCC2] |
| 156 | 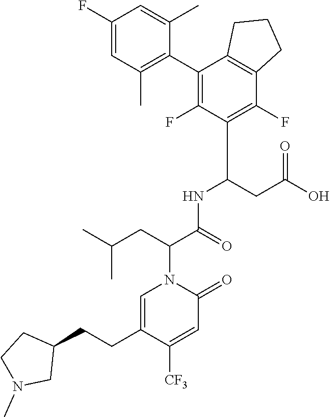 [SMILES: Cc1cc(F)cc(C)c1-c1c(F)c(C(CC(=O)O)NC(=O)C(CC(C)C)n2cc(CC[C@@H]3CCN(C)C3)c(C(F)(F)F)cc2=O)c(F)c2c1CCC2] |
| | 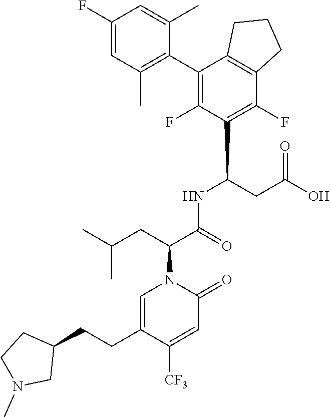 [SMILES: Cc1cc(F)cc(C)c1-c1c(F)c([C@@H](CC(=O)O)NC(=O)[C@H](CC(C)C)n2cc(CC[C@@H]3CCN(C)C3)c(C(F)(F)F)cc2=O)c(F)c2c1CCC2] |
| | 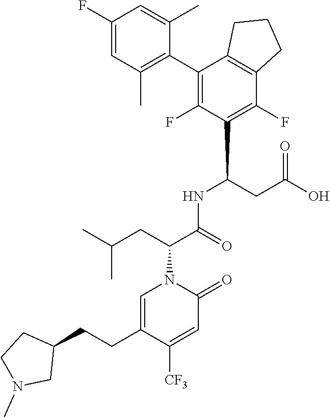 [SMILES: Cc1cc(F)cc(C)c1-c1c(F)c([C@@H](CC(=O)O)NC(=O)[C@@H](CC(C)C)n2cc(CC[C@@H]3CCN(C)C3)c(C(F)(F)F)cc2=O)c(F)c2c1CCC2] |
| | 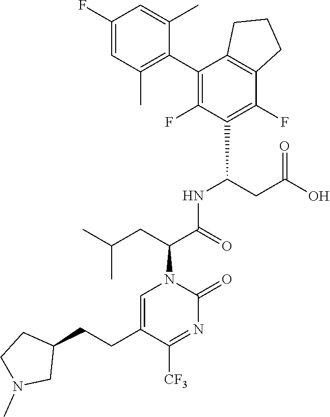 [SMILES: Cc1cc(F)cc(C)c1-c1c(F)c([C@H](CC(=O)O)NC(=O)[C@H](CC(C)C)n2cc(CC[C@@H]3CCN(C)C3)c(C(F)(F)F)nc2=O)c(F)c2c1CCC2] |
| | 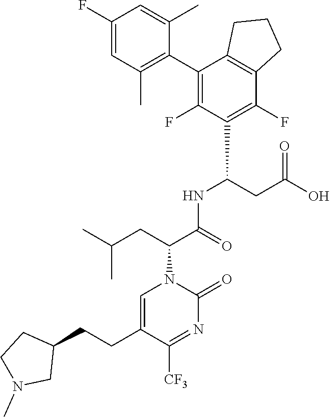 [SMILES: Cc1cc(F)cc(C)c1-c1c(F)c([C@H](CC(=O)O)NC(=O)[C@@H](CC(C)C)n2cc(CC[C@@H]3CCN(C)C3)c(C(F)(F)F)nc2=O)c(F)c2c1CCC2] |
| 157 | 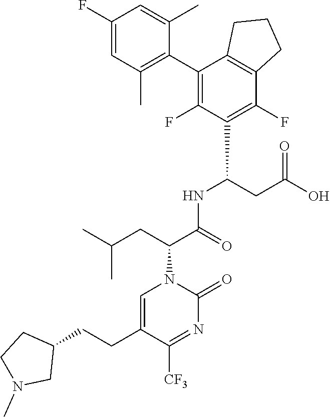 [SMILES: Cc1cc(F)cc(C)c1-c1c(F)c([C@H](CC(=O)O)NC(=O)[C@@H](CC(C)C)n2cc(CC[C@H]3CCN(C)C3)c(C(F)(F)F)nc2=O)c(F)c2c1CCC2] |
| | 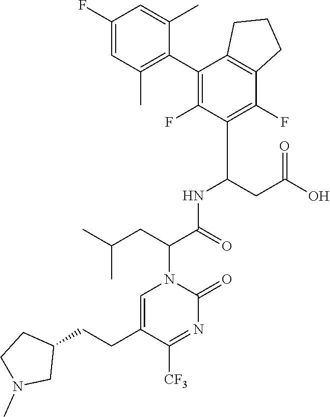 [SMILES: Cc1cc(F)cc(C)c1-c1c(F)c(C(CC(=O)O)NC(=O)C(CC(C)C)n2cc(CC[C@H]3CCN(C)C3)c(C(F)(F)F)nc2=O)c(F)c2c1CCC2] |
| | 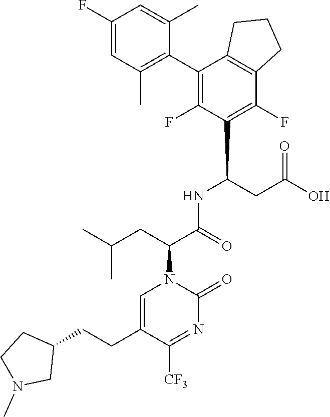 [SMILES: Cc1cc(F)cc(C)c1-c1c(F)c([C@@H](CC(=O)O)NC(=O)[C@H](CC(C)C)n2cc(CC[C@H]3CCN(C)C3)c(C(F)(F)F)nc2=O)c(F)c2c1CCC2] |
| | 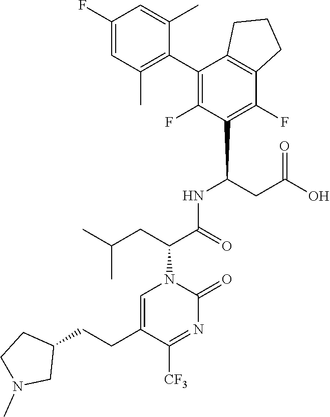 [SMILES: Cc1cc(F)cc(C)c1-c1c(F)c([C@@H](CC(=O)O)NC(=O)[C@@H](CC(C)C)n2cc(CC[C@H]3CCN(C)C3)c(C(F)(F)F)nc2=O)c(F)c2c1CCC2] |
| | 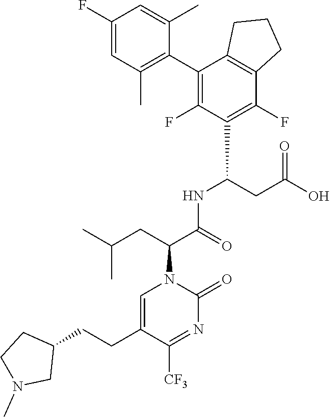 [SMILES: Cc1cc(F)cc(C)c1-c1c(F)c([C@H](CC(=O)O)NC(=O)[C@H](CC(C)C)n2cc(CC[C@H]3CCN(C)C3)c(C(F)(F)F)nc2=O)c(F)c2c1CCC2] |
| 158 | 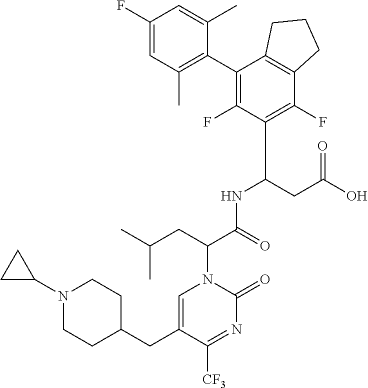 [SMILES: Cc1cc(F)cc(C)c1-c1c(F)c(C(CC(=O)O)NC(=O)C(CC(C)C)n2cc(CC3CCN(C4CC4)CC3)c(C(F)(F)F)nc2=O)c(F)c2c1CCC2] |
| | 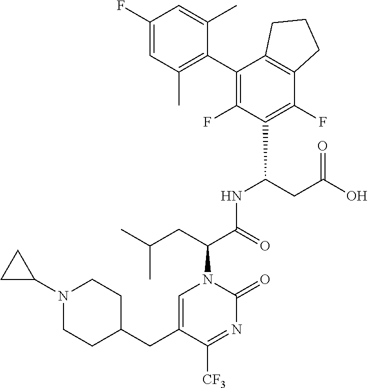 [SMILES: Cc1cc(F)cc(C)c1-c1c(F)c([C@H](CC(=O)O)NC(=O)[C@H](CC(C)C)n2cc(CC3CCN(C4CC4)CC3)c(C(F)(F)F)nc2=O)c(F)c2c1CCC2] |
| | 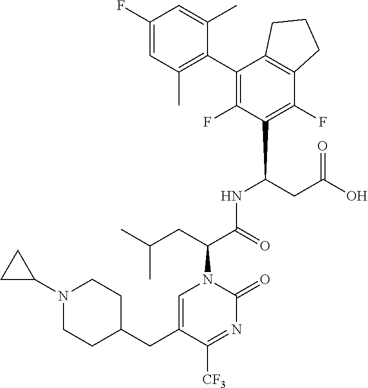 [SMILES: Cc1cc(F)cc(C)c1-c1c(F)c([C@@H](CC(=O)O)NC(=O)[C@H](CC(C)C)n2cc(CC3CCN(C4CC4)CC3)c(C(F)(F)F)nc2=O)c(F)c2c1CCC2] |
| | 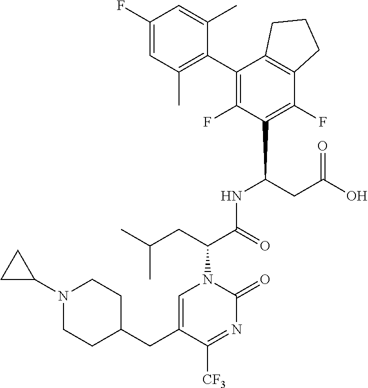 [SMILES: Cc1cc(F)cc(C)c1-c1c(F)c([C@@H](CC(=O)O)NC(=O)[C@@H](CC(C)C)n2cc(CC3CCN(C4CC4)CC3)c(C(F)(F)F)nc2=O)c(F)c2c1CCC2] |
| | 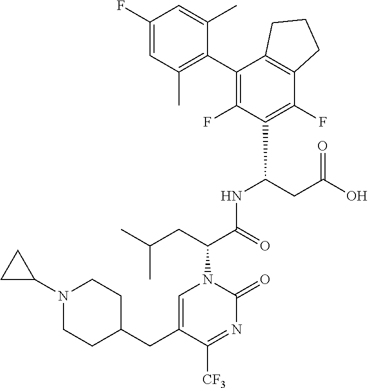 [SMILES: Cc1cc(F)cc(C)c1-c1c(F)c([C@H](CC(=O)O)NC(=O)[C@@H](CC(C)C)n2cc(CC3CCN(C4CC4)CC3)c(C(F)(F)F)nc2=O)c(F)c2c1CCC2] |
| 159 | 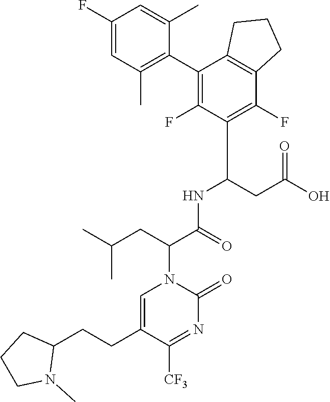 [SMILES: Cc1cc(F)cc(C)c1-c1c(F)c(C(CC(=O)O)NC(=O)C(CC(C)C)n2cc(CCC3CCCN3C)c(C(F)(F)F)nc2=O)c(F)c2c1CCC2] |
| | 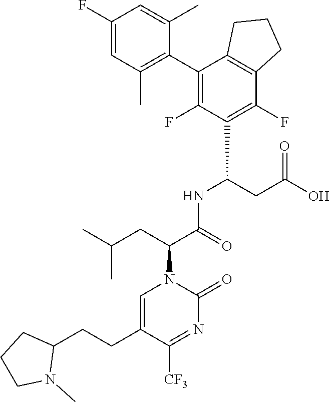 [SMILES: Cc1cc(F)cc(C)c1-c1c(F)c([C@H](CC(=O)O)NC(=O)[C@H](CC(C)C)n2cc(CCC3CCCN3C)c(C(F)(F)F)nc2=O)c(F)c2c1CCC2] |
| | 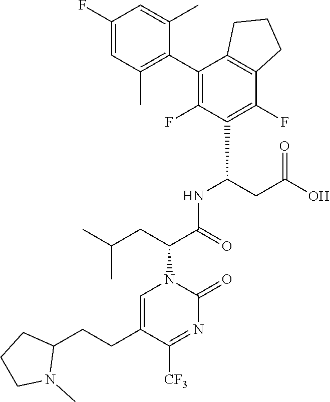 [SMILES: Cc1cc(F)cc(C)c1-c1c(F)c([C@H](CC(=O)O)NC(=O)[C@@H](CC(C)C)n2cc(CCC3CCCN3C)c(C(F)(F)F)nc2=O)c(F)c2c1CCC2] |
| | 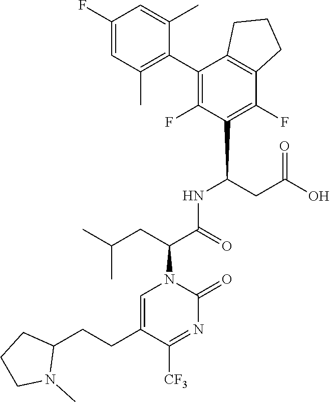 [SMILES: Cc1cc(F)cc(C)c1-c1c(F)c([C@@H](CC(=O)O)NC(=O)[C@H](CC(C)C)n2cc(CCC3CCCN3C)c(C(F)(F)F)nc2=O)c(F)c2c1CCC2] |
| | 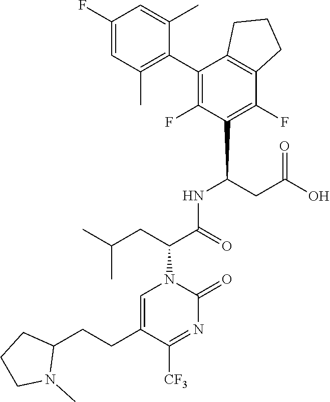 [SMILES: Cc1cc(F)cc(C)c1-c1c(F)c([C@@H](CC(=O)O)NC(=O)[C@@H](CC(C)C)n2cc(CCC3CCCN3C)c(C(F)(F)F)nc2=O)c(F)c2c1CCC2] |
| 160 | 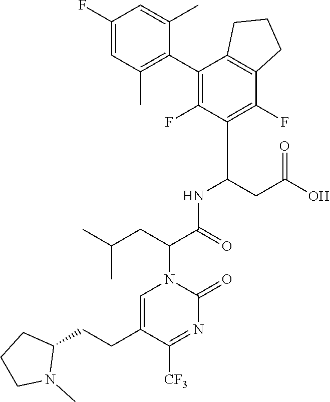 [SMILES: Cc1cc(F)cc(C)c1-c1c(F)c(C(CC(=O)O)NC(=O)C(CC(C)C)n2cc(CC[C@H]3CCCN3C)c(C(F)(F)F)nc2=O)c(F)c2c1CCC2] |
| | 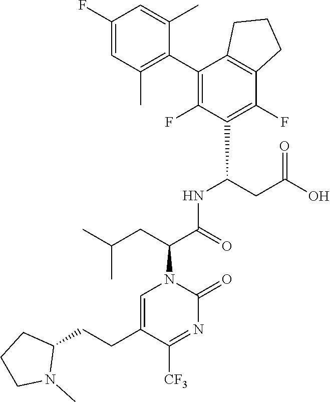 [SMILES: Cc1cc(F)cc(C)c1-c1c(F)c([C@H](CC(=O)O)NC(=O)[C@H](CC(C)C)n2cc(CC[C@H]3CCCN3C)c(C(F)(F)F)nc2=O)c(F)c2c1CCC2] |
| | 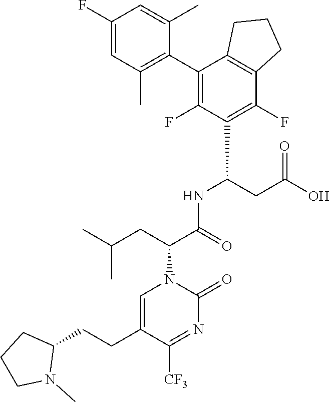 [SMILES: Cc1cc(F)cc(C)c1-c1c(F)c([C@H](CC(=O)O)NC(=O)[C@@H](CC(C)C)n2cc(CC[C@H]3CCCN3C)c(C(F)(F)F)nc2=O)c(F)c2c1CCC2] |
| | 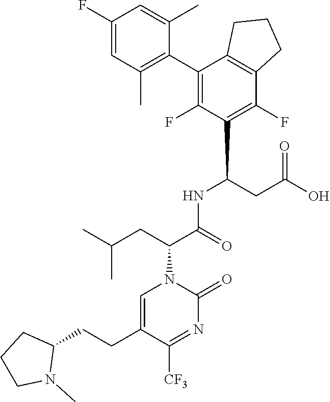 [SMILES: Cc1cc(F)cc(C)c1-c1c(F)c([C@@H](CC(=O)O)NC(=O)[C@@H](CC(C)C)n2cc(CC[C@H]3CCCN3C)c(C(F)(F)F)nc2=O)c(F)c2c1CCC2] |
| | 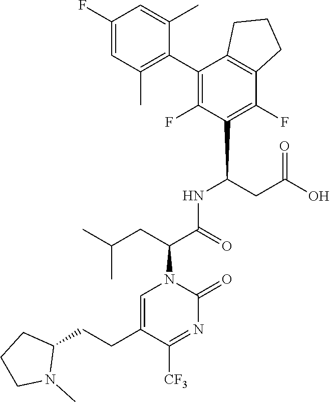 [SMILES: Cc1cc(F)cc(C)c1-c1c(F)c([C@@H](CC(=O)O)NC(=O)[C@H](CC(C)C)n2cc(CC[C@H]3CCCN3C)c(C(F)(F)F)nc2=O)c(F)c2c1CCC2] |
| 161 | 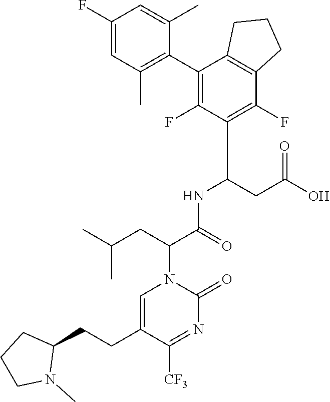 [SMILES: Cc1cc(F)cc(C)c1-c1c(F)c(C(CC(=O)O)NC(=O)C(CC(C)C)n2cc(CC[C@@H]3CCCN3C)c(C(F)(F)F)nc2=O)c(F)c2c1CCC2] |
| | 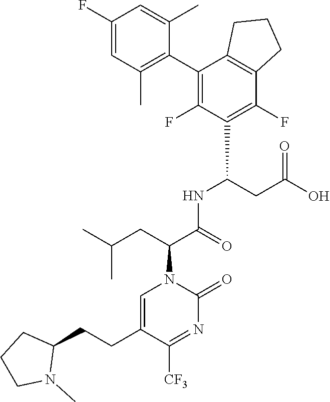 [SMILES: Cc1cc(F)cc(C)c1-c1c(F)c([C@H](CC(=O)O)NC(=O)[C@H](CC(C)C)n2cc(CC[C@@H]3CCCN3C)c(C(F)(F)F)nc2=O)c(F)c2c1CCC2] |
| | 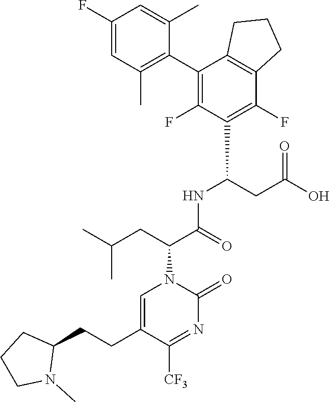 [SMILES: Cc1cc(F)cc(C)c1-c1c(F)c([C@H](CC(=O)O)NC(=O)[C@@H](CC(C)C)n2cc(CC[C@@H]3CCCN3C)c(C(F)(F)F)nc2=O)c(F)c2c1CCC2] |
| | 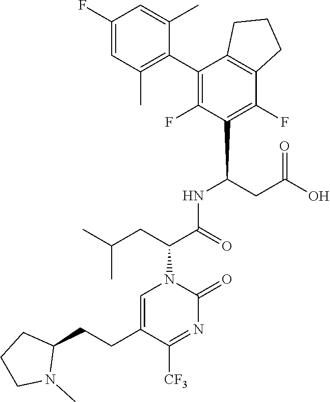 [SMILES: Cc1cc(F)cc(C)c1-c1c(F)c([C@@H](CC(=O)O)NC(=O)[C@@H](CC(C)C)n2cc(CC[C@@H]3CCCN3C)c(C(F)(F)F)nc2=O)c(F)c2c1CCC2] |
| | 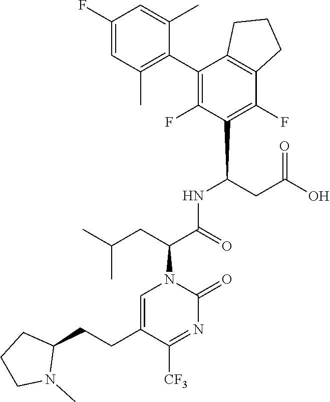 [SMILES: Cc1cc(F)cc(C)c1-c1c(F)c([C@@H](CC(=O)O)NC(=O)[C@H](CC(C)C)n2cc(CC[C@@H]3CCCN3C)c(C(F)(F)F)nc2=O)c(F)c2c1CCC2] |
| 162 | 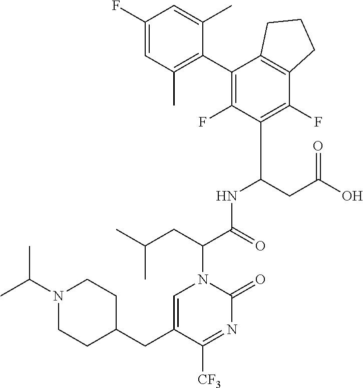 [SMILES: Cc1cc(F)cc(C)c1-c1c(F)c(C(CC(=O)O)NC(=O)C(CC(C)C)n2cc(CC3CCN(C(C)C)CC3)c(C(F)(F)F)nc2=O)c(F)c2c1CCC2] |
| | 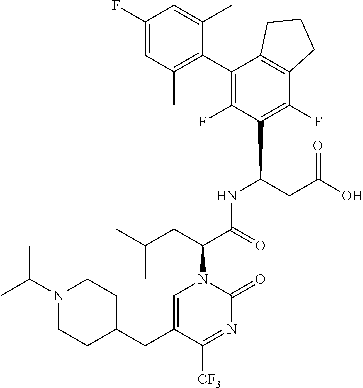 [SMILES: Cc1cc(F)cc(C)c1-c1c(F)c([C@@H](CC(=O)O)NC(=O)[C@H](CC(C)C)n2cc(CC3CCN(C(C)C)CC3)c(C(F)(F)F)nc2=O)c(F)c2c1CCC2] |
| | 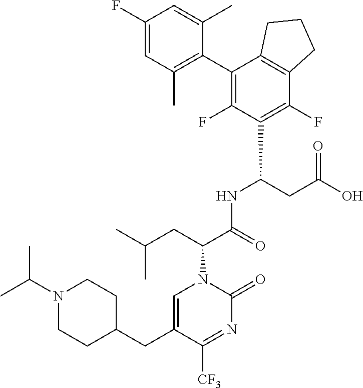 [SMILES: Cc1cc(F)cc(C)c1-c1c(F)c([C@H](CC(=O)O)NC(=O)[C@@H](CC(C)C)n2cc(CC3CCN(C(C)C)CC3)c(C(F)(F)F)nc2=O)c(F)c2c1CCC2] |
| | 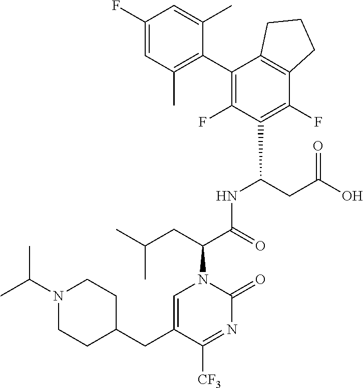 [SMILES: Cc1cc(F)cc(C)c1-c1c(F)c([C@H](CC(=O)O)NC(=O)[C@H](CC(C)C)n2cc(CC3CCN(C(C)C)CC3)c(C(F)(F)F)nc2=O)c(F)c2c1CCC2] |
| | 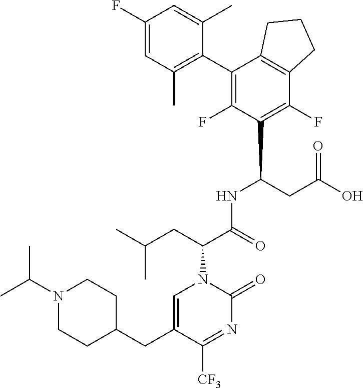 [SMILES: Cc1cc(F)cc(C)c1-c1c(F)c([C@@H](CC(=O)O)NC(=O)[C@@H](CC(C)C)n2cc(CC3CCN(C(C)C)CC3)c(C(F)(F)F)nc2=O)c(F)c2c1CCC2] |
| 163 | 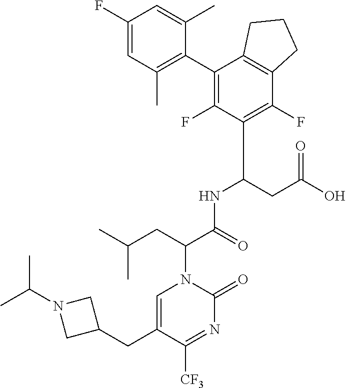 [SMILES: Cc1cc(F)cc(C)c1-c1c(F)c(C(CC(=O)O)NC(=O)C(CC(C)C)n2cc(CC3CN(C(C)C)C3)c(C(F)(F)F)nc2=O)c(F)c2c1CCC2] |
| | 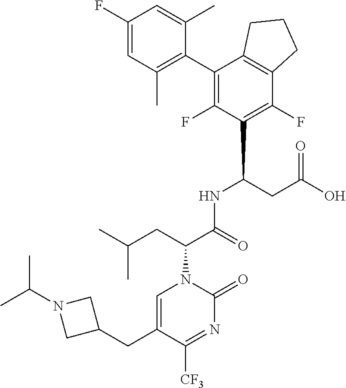 [SMILES: Cc1cc(F)cc(C)c1-c1c(F)c([C@@H](CC(=O)O)NC(=O)[C@@H](CC(C)C)n2cc(CC3CN(C(C)C)C3)c(C(F)(F)F)nc2=O)c(F)c2c1CCC2] |
| | 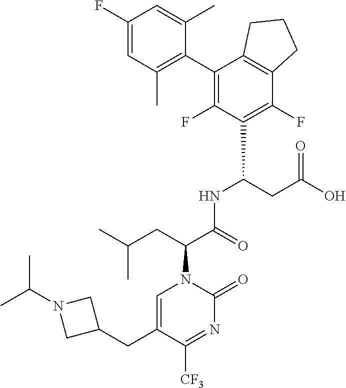 [SMILES: Cc1cc(F)cc(C)c1-c1c(F)c([C@H](CC(=O)O)NC(=O)[C@H](CC(C)C)n2cc(CC3CN(C(C)C)C3)c(C(F)(F)F)nc2=O)c(F)c2c1CCC2] |
| | 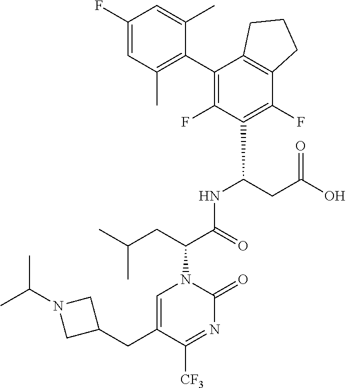 [SMILES: Cc1cc(F)cc(C)c1-c1c(F)c([C@H](CC(=O)O)NC(=O)[C@@H](CC(C)C)n2cc(CC3CN(C(C)C)C3)c(C(F)(F)F)nc2=O)c(F)c2c1CCC2] |
| | 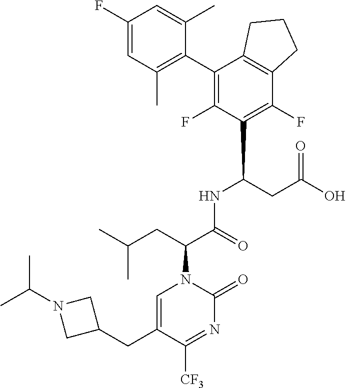 [SMILES: Cc1cc(F)cc(C)c1-c1c(F)c([C@@H](CC(=O)O)NC(=O)[C@H](CC(C)C)n2cc(CC3CN(C(C)C)C3)c(C(F)(F)F)nc2=O)c(F)c2c1CCC2] |
| 164 | 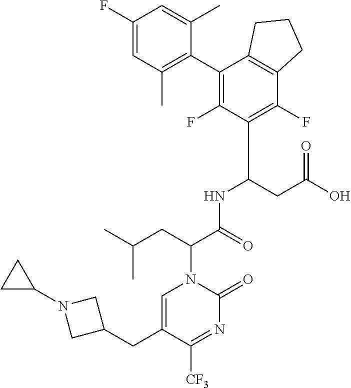 [SMILES: Cc1cc(F)cc(C)c1-c1c(F)c(C(CC(=O)O)NC(=O)C(CC(C)C)n2cc(CC3CN(C4CC4)C3)c(C(F)(F)F)nc2=O)c(F)c2c1CCC2] |
| | 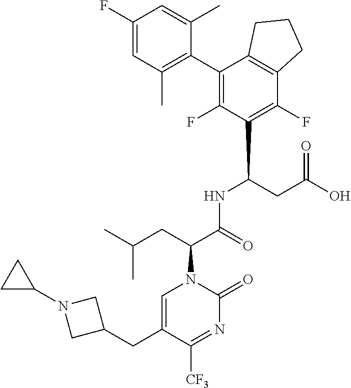 [SMILES: Cc1cc(F)cc(C)c1-c1c(F)c([C@@H](CC(=O)O)NC(=O)[C@H](CC(C)C)n2cc(CC3CN(C4CC4)C3)c(C(F)(F)F)nc2=O)c(F)c2c1CCC2] |
| | 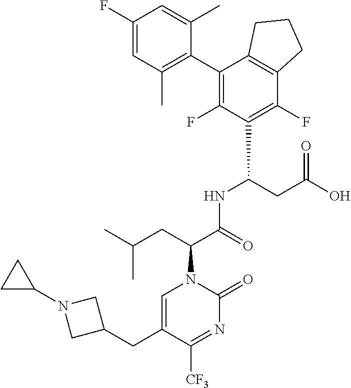 [SMILES: Cc1cc(F)cc(C)c1-c1c(F)c([C@H](CC(=O)O)NC(=O)[C@H](CC(C)C)n2cc(CC3CN(C4CC4)C3)c(C(F)(F)F)nc2=O)c(F)c2c1CCC2] |
| | 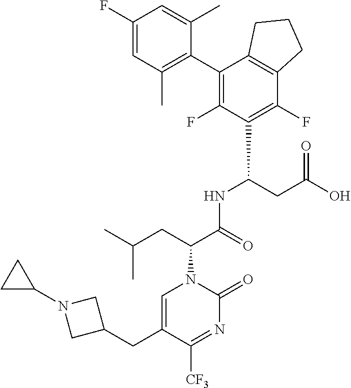 [SMILES: Cc1cc(F)cc(C)c1-c1c(F)c([C@H](CC(=O)O)NC(=O)[C@@H](CC(C)C)n2cc(CC3CN(C4CC4)C3)c(C(F)(F)F)nc2=O)c(F)c2c1CCC2] |
| | 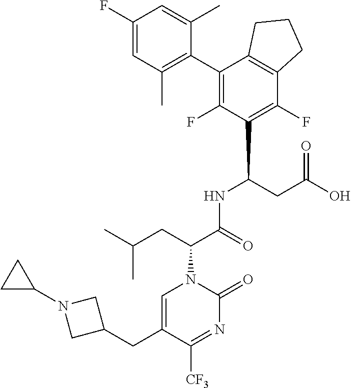 [SMILES: Cc1cc(F)cc(C)c1-c1c(F)c([C@@H](CC(=O)O)NC(=O)[C@@H](CC(C)C)n2cc(CC3CN(C4CC4)C3)c(C(F)(F)F)nc2=O)c(F)c2c1CCC2] |
| 165 | 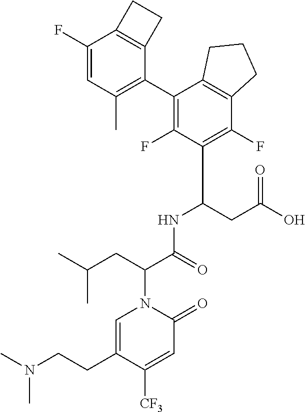 [SMILES: Cc1cc(F)c2c(c1-c1c(F)c(C(CC(=O)O)NC(=O)C(CC(C)C)n3cc(CCN(C)C)c(C(F)(F)F)cc3=O)c(F)c3c1CCC3)CC2] |
| | 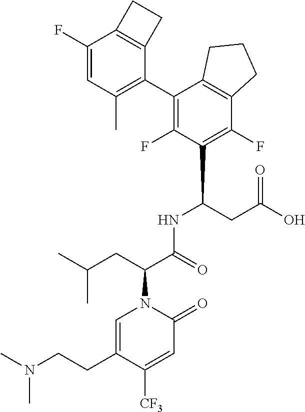 [SMILES: Cc1cc(F)c2c(c1-c1c(F)c([C@@H](CC(=O)O)NC(=O)[C@H](CC(C)C)n3cc(CCN(C)C)c(C(F)(F)F)cc3=O)c(F)c3c1CCC3)CC2] |
| | 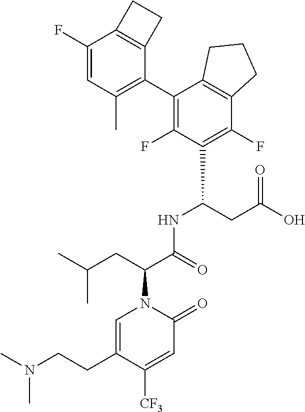 [SMILES: Cc1cc(F)c2c(c1-c1c(F)c([C@H](CC(=O)O)NC(=O)[C@H](CC(C)C)n3cc(CCN(C)C)c(C(F)(F)F)cc3=O)c(F)c3c1CCC3)CC2] |
| | 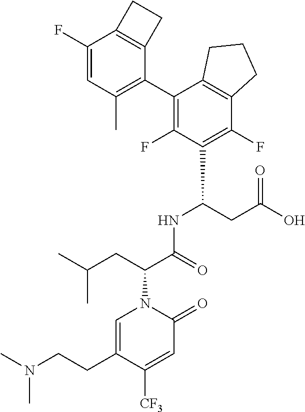 [SMILES: Cc1cc(F)c2c(c1-c1c(F)c([C@H](CC(=O)O)NC(=O)[C@@H](CC(C)C)n3cc(CCN(C)C)c(C(F)(F)F)cc3=O)c(F)c3c1CCC3)CC2] |
| | 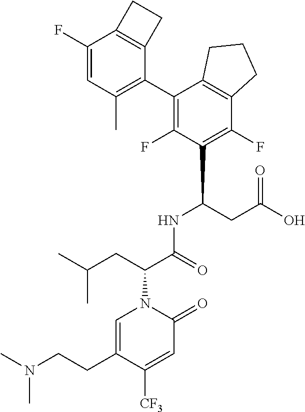 [SMILES: Cc1cc(F)c2c(c1-c1c(F)c([C@@H](CC(=O)O)NC(=O)[C@@H](CC(C)C)n3cc(CCN(C)C)c(C(F)(F)F)cc3=O)c(F)c3c1CCC3)CC2] |
| 166 | 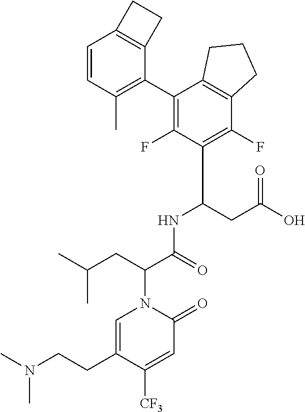 [SMILES: Cc1ccc2c(c1-c1c(F)c(C(CC(=O)O)NC(=O)C(CC(C)C)n3cc(CCN(C)C)c(C(F)(F)F)cc3=O)c(F)c3c1CCC3)CC2] |
| | 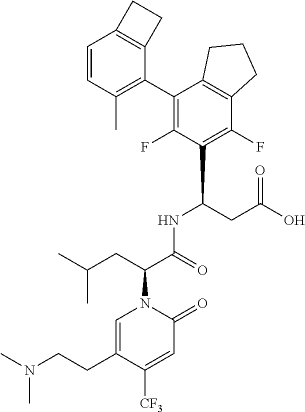 [SMILES: Cc1ccc2c(c1-c1c(F)c([C@@H](CC(=O)O)NC(=O)[C@H](CC(C)C)n3cc(CCN(C)C)c(C(F)(F)F)cc3=O)c(F)c3c1CCC3)CC2] |
| | 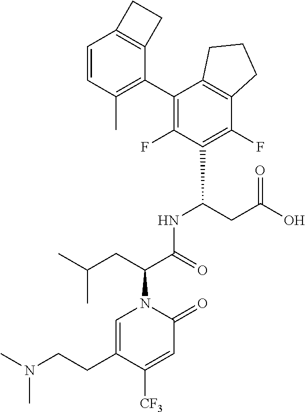 [SMILES: Cc1ccc2c(c1-c1c(F)c([C@H](CC(=O)O)NC(=O)[C@H](CC(C)C)n3cc(CCN(C)C)c(C(F)(F)F)cc3=O)c(F)c3c1CCC3)CC2] |
| | 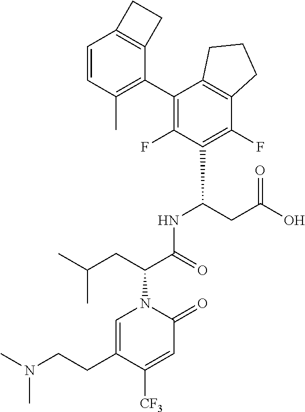 [SMILES: Cc1ccc2c(c1-c1c(F)c([C@H](CC(=O)O)NC(=O)[C@@H](CC(C)C)n3cc(CCN(C)C)c(C(F)(F)F)cc3=O)c(F)c3c1CCC3)CC2] |
| | 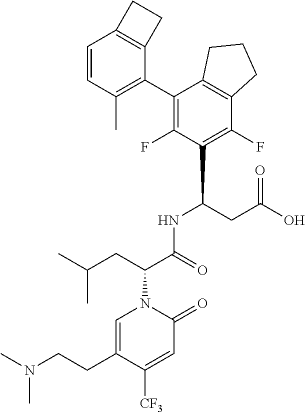 [SMILES: Cc1ccc2c(c1-c1c(F)c([C@@H](CC(=O)O)NC(=O)[C@@H](CC(C)C)n3cc(CCN(C)C)c(C(F)(F)F)cc3=O)c(F)c3c1CCC3)CC2] |
| 167 | 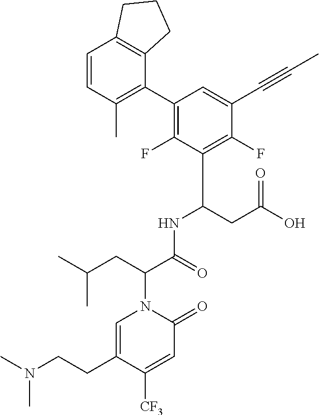 [SMILES: CC#Cc1cc(-c2c(C)ccc3c2CCC3)c(F)c(C(CC(=O)O)NC(=O)C(CC(C)C)n2cc(CCN(C)C)c(C(F)(F)F)cc2=O)c1F] |
| | 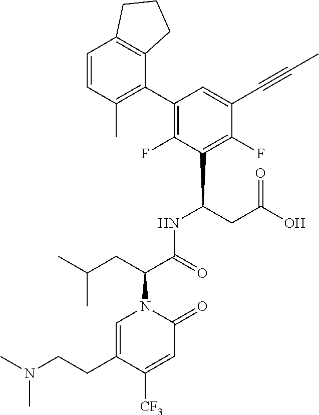 [SMILES: CC#Cc1cc(-c2c(C)ccc3c2CCC3)c(F)c([C@@H](CC(=O)O)NC(=O)[C@H](CC(C)C)n2cc(CCN(C)C)c(C(F)(F)F)cc2=O)c1F] |
| | 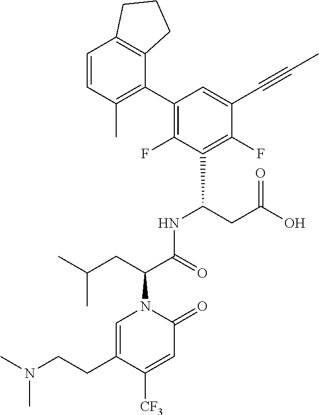 [SMILES: CC#Cc1cc(-c2c(C)ccc3c2CCC3)c(F)c([C@H](CC(=O)O)NC(=O)[C@H](CC(C)C)n2cc(CCN(C)C)c(C(F)(F)F)cc2=O)c1F] |
| | 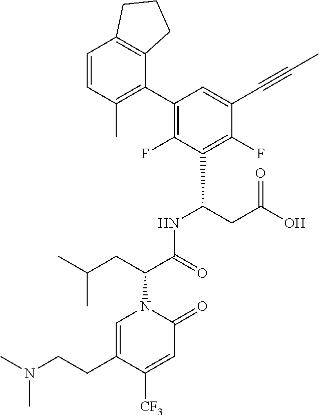 [SMILES: CC#Cc1cc(-c2c(C)ccc3c2CCC3)c(F)c([C@H](CC(=O)O)NC(=O)[C@@H](CC(C)C)n2cc(CCN(C)C)c(C(F)(F)F)cc2=O)c1F] |
| | 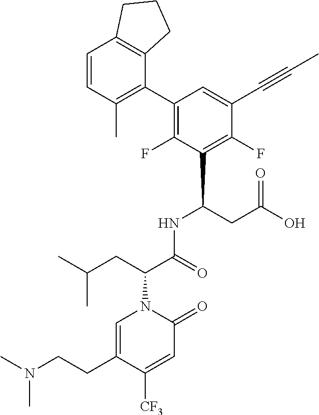 [SMILES: CC#Cc1cc(-c2c(C)ccc3c2CCC3)c(F)c([C@@H](CC(=O)O)NC(=O)[C@@H](CC(C)C)n2cc(CCN(C)C)c(C(F)(F)F)cc2=O)c1F] |
| 168 | 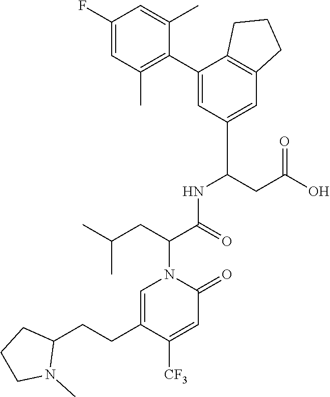 [SMILES: Cc1cc(F)cc(C)c1-c1cc(C(CC(=O)O)NC(=O)C(CC(C)C)n2cc(CCC3CCCN3C)c(C(F)(F)F)cc2=O)cc2c1CCC2] |
| | 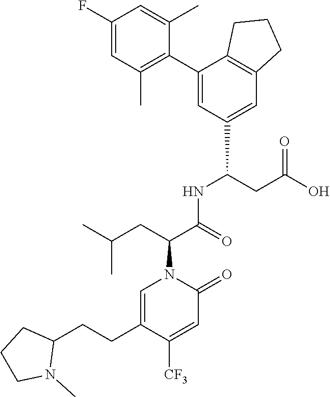 [SMILES: Cc1cc(F)cc(C)c1-c1cc([C@H](CC(=O)O)NC(=O)[C@H](CC(C)C)n2cc(CCC3CCCN3C)c(C(F)(F)F)cc2=O)cc2c1CCC2] |
| | 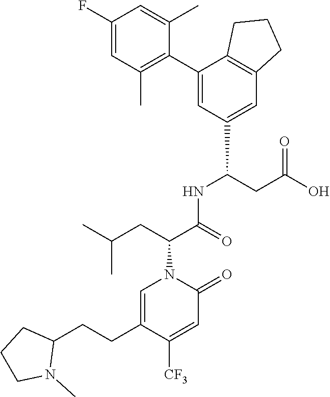 [SMILES: Cc1cc(F)cc(C)c1-c1cc([C@H](CC(=O)O)NC(=O)[C@@H](CC(C)C)n2cc(CCC3CCCN3C)c(C(F)(F)F)cc2=O)cc2c1CCC2] |
| | 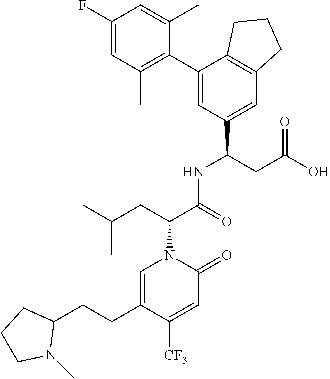 [SMILES: Cc1cc(F)cc(C)c1-c1cc([C@@H](CC(=O)O)NC(=O)[C@@H](CC(C)C)n2cc(CCC3CCCN3C)c(C(F)(F)F)cc2=O)cc2c1CCC2] |
| | 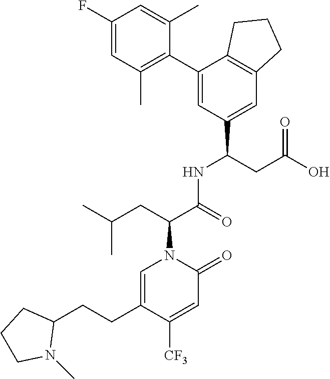 [SMILES: Cc1cc(F)cc(C)c1-c1cc([C@@H](CC(=O)O)NC(=O)[C@H](CC(C)C)n2cc(CCC3CCCN3C)c(C(F)(F)F)cc2=O)cc2c1CCC2] |
| 169 | 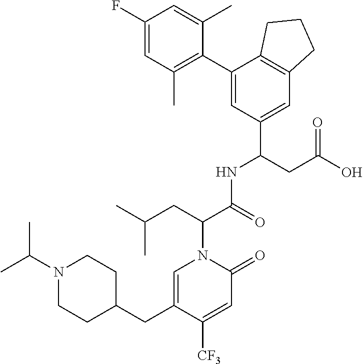 [SMILES: Cc1cc(F)cc(C)c1-c1cc(C(CC(=O)O)NC(=O)C(CC(C)C)n2cc(CC3CCN(C(C)C)CC3)c(C(F)(F)F)cc2=O)cc2c1CCC2] |
| | 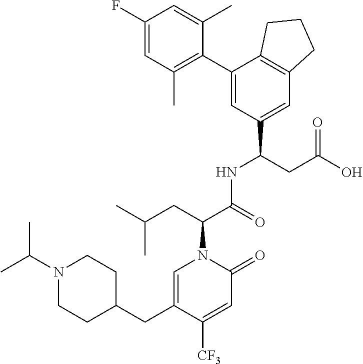 [SMILES: Cc1cc(F)cc(C)c1-c1cc([C@@H](CC(=O)O)NC(=O)[C@H](CC(C)C)n2cc(CC3CCN(C(C)C)CC3)c(C(F)(F)F)cc2=O)cc2c1CCC2] |
| | 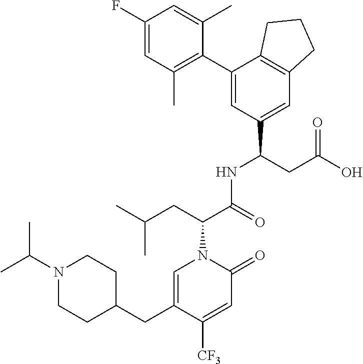 [SMILES: Cc1cc(F)cc(C)c1-c1cc([C@@H](CC(=O)O)NC(=O)[C@@H](CC(C)C)n2cc(CC3CCN(C(C)C)CC3)c(C(F)(F)F)cc2=O)cc2c1CCC2] |
| | 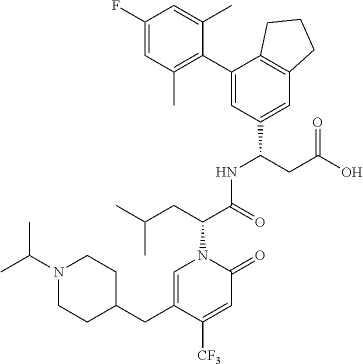 [SMILES: Cc1cc(F)cc(C)c1-c1cc([C@H](CC(=O)O)NC(=O)[C@@H](CC(C)C)n2cc(CC3CCN(C(C)C)CC3)c(C(F)(F)F)cc2=O)cc2c1CCC2] |
| | 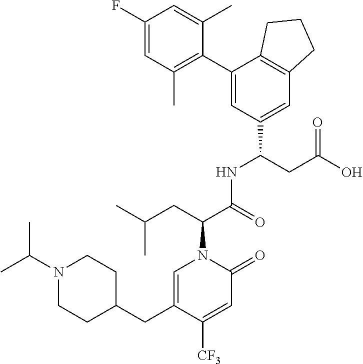 [SMILES: Cc1cc(F)cc(C)c1-c1cc([C@H](CC(=O)O)NC(=O)[C@H](CC(C)C)n2cc(CC3CCN(C(C)C)CC3)c(C(F)(F)F)cc2=O)cc2c1CCC2] |
| 170 | 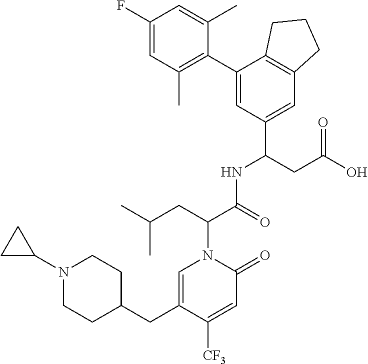 [SMILES: Cc1cc(F)cc(C)c1-c1cc(C(CC(=O)O)NC(=O)C(CC(C)C)n2cc(CC3CCN(C4CC4)CC3)c(C(F)(F)F)cc2=O)cc2c1CCC2] |
| | 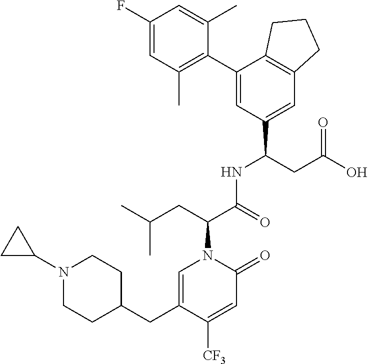 [SMILES: Cc1cc(F)cc(C)c1-c1cc([C@@H](CC(=O)O)NC(=O)[C@H](CC(C)C)n2cc(CC3CCN(C4CC4)CC3)c(C(F)(F)F)cc2=O)cc2c1CCC2] |
| | 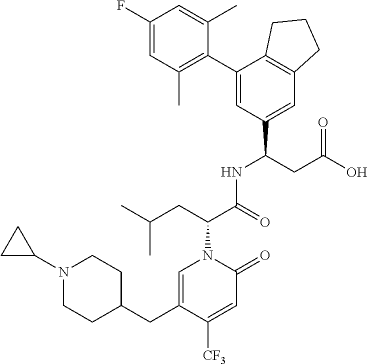 [SMILES: Cc1cc(F)cc(C)c1-c1cc([C@@H](CC(=O)O)NC(=O)[C@@H](CC(C)C)n2cc(CC3CCN(C4CC4)CC3)c(C(F)(F)F)cc2=O)cc2c1CCC2] |
| | 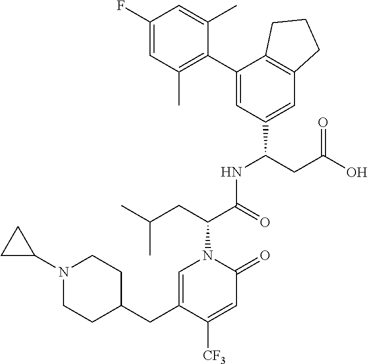 [SMILES: Cc1cc(F)cc(C)c1-c1cc([C@H](CC(=O)O)NC(=O)[C@@H](CC(C)C)n2cc(CC3CCN(C4CC4)CC3)c(C(F)(F)F)cc2=O)cc2c1CCC2] |
| | 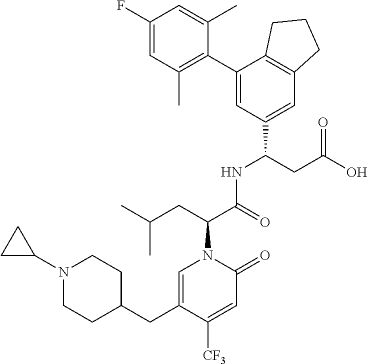 [SMILES: Cc1cc(F)cc(C)c1-c1cc([C@H](CC(=O)O)NC(=O)[C@H](CC(C)C)n2cc(CC3CCN(C4CC4)CC3)c(C(F)(F)F)cc2=O)cc2c1CCC2] |
| 171 | 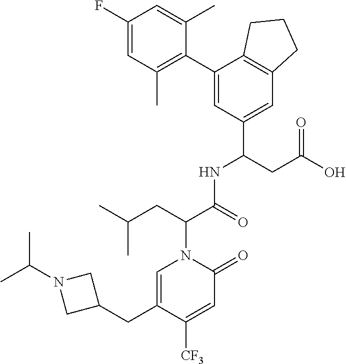 [SMILES: Cc1cc(F)cc(C)c1-c1cc(C(CC(=O)O)NC(=O)C(CC(C)C)n2cc(CC3CN(C(C)C)C3)c(C(F)(F)F)cc2=O)cc2c1CCC2] |
| | 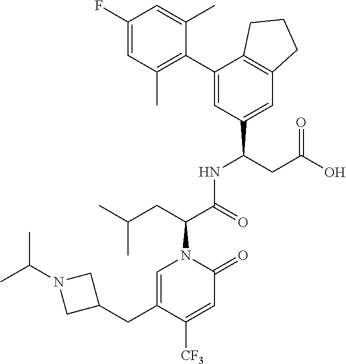 [SMILES: Cc1cc(F)cc(C)c1-c1cc([C@@H](CC(=O)O)NC(=O)[C@H](CC(C)C)n2cc(CC3CN(C(C)C)C3)c(C(F)(F)F)cc2=O)cc2c1CCC2] |
| | 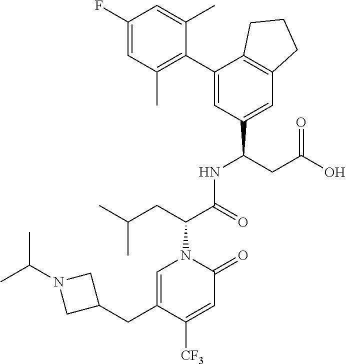 [SMILES: Cc1cc(F)cc(C)c1-c1cc([C@@H](CC(=O)O)NC(=O)[C@@H](CC(C)C)n2cc(CC3CN(C(C)C)C3)c(C(F)(F)F)cc2=O)cc2c1CCC2] |
| | 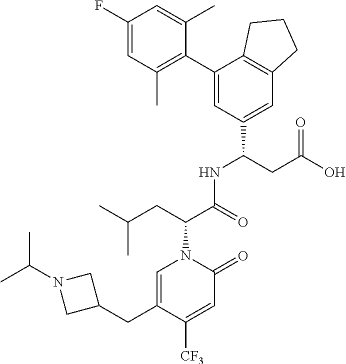 [SMILES: Cc1cc(F)cc(C)c1-c1cc([C@H](CC(=O)O)NC(=O)[C@@H](CC(C)C)n2cc(CC3CN(C(C)C)C3)c(C(F)(F)F)cc2=O)cc2c1CCC2] |
| | 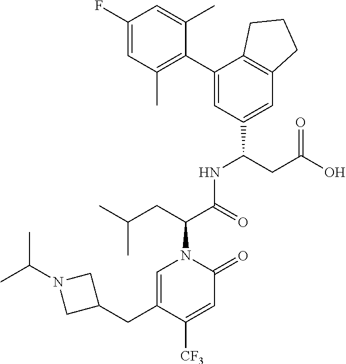 [SMILES: Cc1cc(F)cc(C)c1-c1cc([C@H](CC(=O)O)NC(=O)[C@H](CC(C)C)n2cc(CC3CN(C(C)C)C3)c(C(F)(F)F)cc2=O)cc2c1CCC2] |
| 172 | 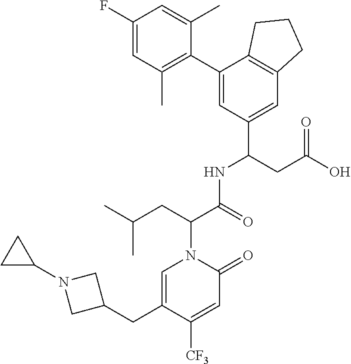 [SMILES: Cc1cc(F)cc(C)c1-c1cc(C(CC(=O)O)NC(=O)C(CC(C)C)n2cc(CC3CN(C4CC4)C3)c(C(F)(F)F)cc2=O)cc2c1CCC2] |
| | 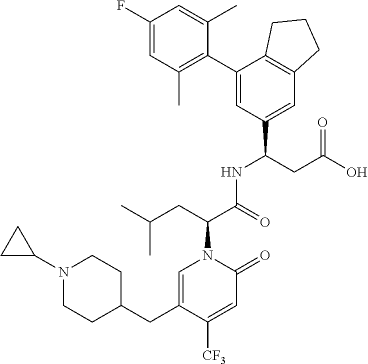 [SMILES: Cc1cc(F)cc(C)c1-c1cc([C@@H](CC(=O)O)NC(=O)[C@H](CC(C)C)n2cc(CC3CCN(C4CC4)CC3)c(C(F)(F)F)cc2=O)cc2c1CCC2] |
| | 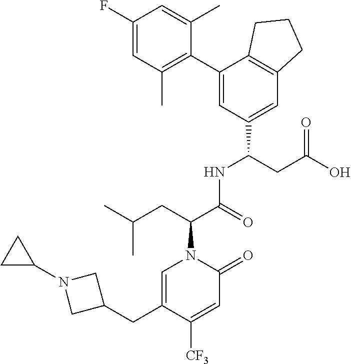 [SMILES: Cc1cc(F)cc(C)c1-c1cc([C@H](CC(=O)O)NC(=O)[C@H](CC(C)C)n2cc(CC3CN(C4CC4)C3)c(C(F)(F)F)cc2=O)cc2c1CCC2] |
| | 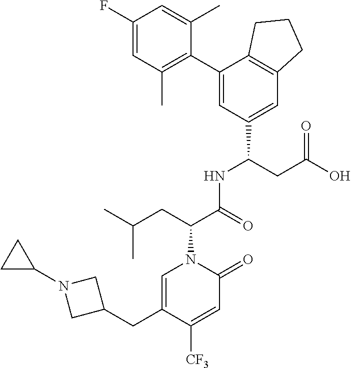 [SMILES: Cc1cc(F)cc(C)c1-c1cc([C@H](CC(=O)O)NC(=O)[C@@H](CC(C)C)n2cc(CC3CN(C4CC4)C3)c(C(F)(F)F)cc2=O)cc2c1CCC2] |
| | 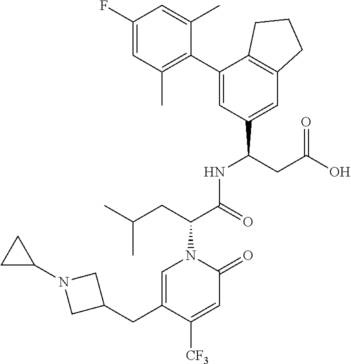 [SMILES: Cc1cc(F)cc(C)c1-c1cc([C@@H](CC(=O)O)NC(=O)[C@@H](CC(C)C)n2cc(CC3CN(C4CC4)C3)c(C(F)(F)F)cc2=O)cc2c1CCC2] |
| 173 | 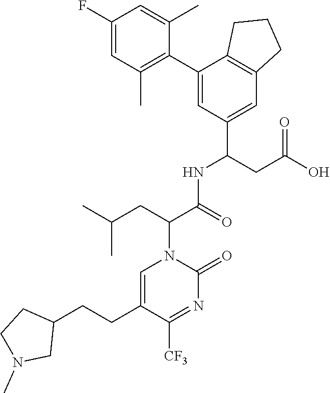 [SMILES: Cc1cc(F)cc(C)c1-c1cc(C(CC(=O)O)NC(=O)C(CC(C)C)n2cc(CCC3CCN(C)C3)c(C(F)(F)F)nc2=O)cc2c1CCC2] |
| | 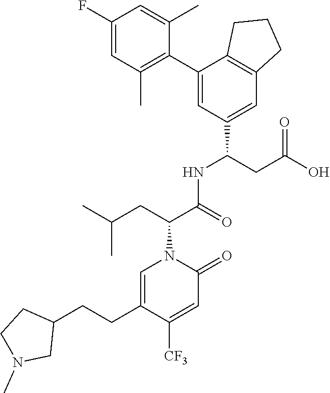 [SMILES: Cc1cc(F)cc(C)c1-c1cc([C@H](CC(=O)O)NC(=O)[C@@H](CC(C)C)n2cc(CCC3CCN(C)C3)c(C(F)(F)F)cc2=O)cc2c1CCC2] |
| | 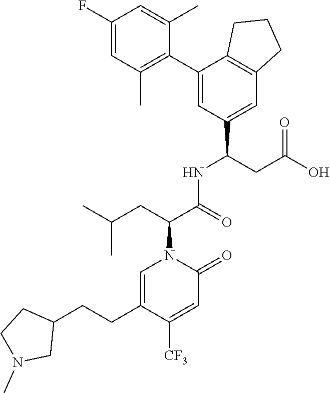 [SMILES: Cc1cc(F)cc(C)c1-c1cc([C@@H](CC(=O)O)NC(=O)[C@H](CC(C)C)n2cc(CCC3CCN(C)C3)c(C(F)(F)F)cc2=O)cc2c1CCC2] |
| | 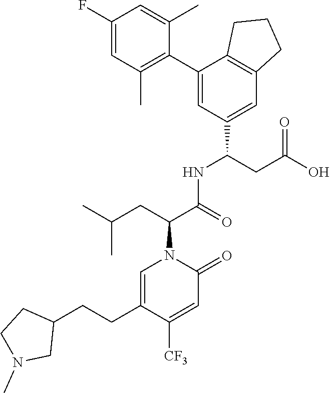 [SMILES: Cc1cc(F)cc(C)c1-c1cc([C@H](CC(=O)O)NC(=O)[C@H](CC(C)C)n2cc(CCC3CCN(C)C3)c(C(F)(F)F)cc2=O)cc2c1CCC2] |
| | 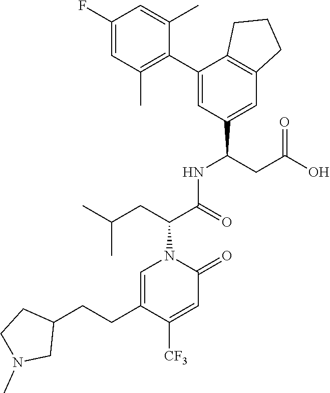 [SMILES: Cc1cc(F)cc(C)c1-c1cc([C@@H](CC(=O)O)NC(=O)[C@@H](CC(C)C)n2cc(CCC3CCN(C)C3)c(C(F)(F)F)cc2=O)cc2c1CCC2] |
| 174 | 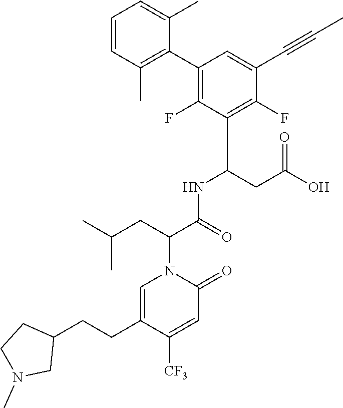 [SMILES: CC#Cc1cc(-c2c(C)cccc2C)c(F)c(C(CC(=O)O)NC(=O)C(CC(C)C)n2cc(CCC3CCN(C)C3)c(C(F)(F)F)cc2=O)c1F] |
| | 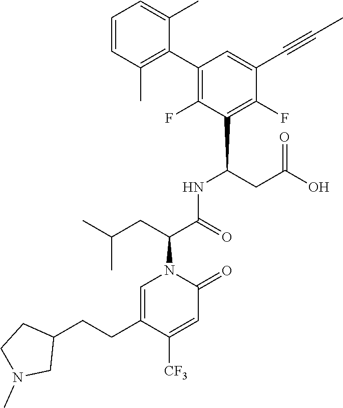 [SMILES: CC#Cc1cc(-c2c(C)cccc2C)c(F)c([C@@H](CC(=O)O)NC(=O)[C@H](CC(C)C)n2cc(CCC3CCN(C)C3)c(C(F)(F)F)cc2=O)c1F] |
| | 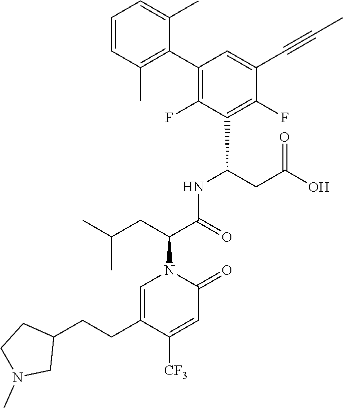 [SMILES: CC#Cc1cc(-c2c(C)cccc2C)c(F)c([C@H](CC(=O)O)NC(=O)[C@H](CC(C)C)n2cc(CCC3CCN(C)C3)c(C(F)(F)F)cc2=O)c1F] |
| | 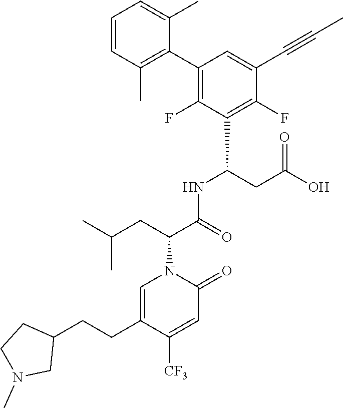 [SMILES: CC#Cc1cc(-c2c(C)cccc2C)c(F)c([C@H](CC(=O)O)NC(=O)[C@@H](CC(C)C)n2cc(CCC3CCN(C)C3)c(C(F)(F)F)cc2=O)c1F] |
| | 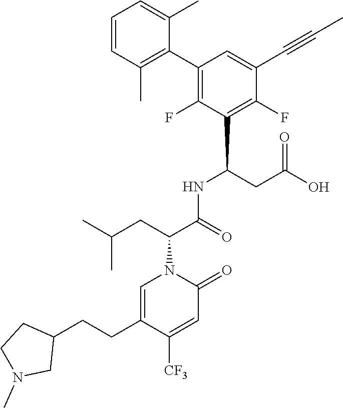 [SMILES: CC#Cc1cc(-c2c(C)cccc2C)c(F)c([C@@H](CC(=O)O)NC(=O)[C@@H](CC(C)C)n2cc(CCC3CCN(C)C3)c(C(F)(F)F)cc2=O)c1F] |
| 175 | 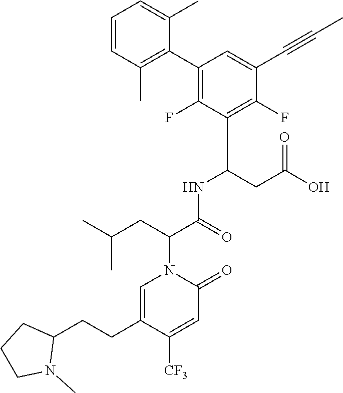 [SMILES: CC#Cc1cc(-c2c(C)cccc2C)c(F)c(C(CC(=O)O)NC(=O)C(CC(C)C)n2cc(CCC3CCCN3C)c(C(F)(F)F)cc2=O)c1F] |
| | 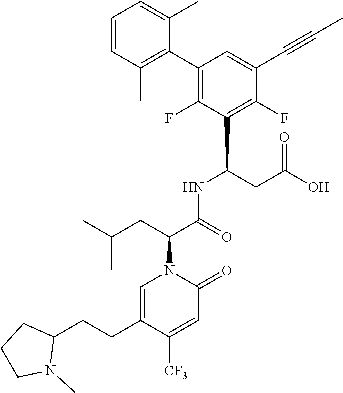 [SMILES: CC#Cc1cc(-c2c(C)cccc2C)c(F)c([C@@H](CC(=O)O)NC(=O)[C@H](CC(C)C)n2cc(CCC3CCCN3C)c(C(F)(F)F)cc2=O)c1F] |
| | 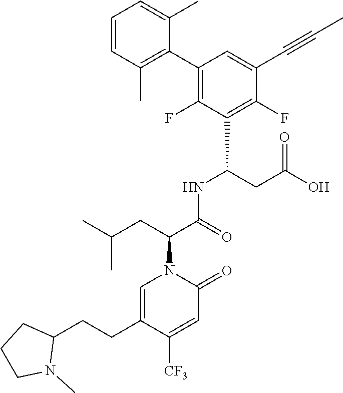 [SMILES: CC#Cc1cc(-c2c(C)cccc2C)c(F)c([C@H](CC(=O)O)NC(=O)[C@H](CC(C)C)n2cc(CCC3CCCN3C)c(C(F)(F)F)cc2=O)c1F] |
| | 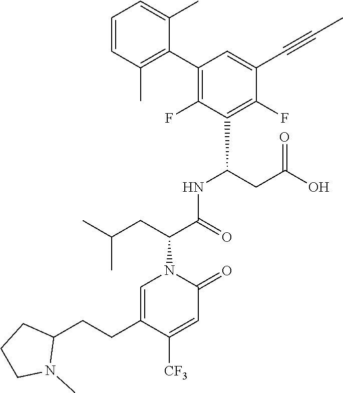 [SMILES: CC#Cc1cc(-c2c(C)cccc2C)c(F)c([C@H](CC(=O)O)NC(=O)[C@@H](CC(C)C)n2cc(CCC3CCCN3C)c(C(F)(F)F)cc2=O)c1F] |
| | 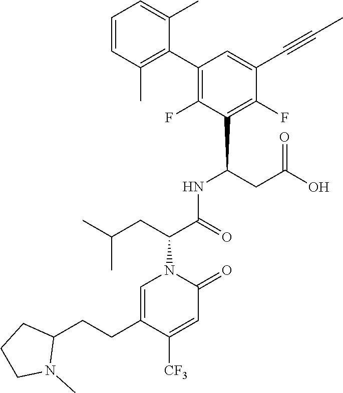 [SMILES: CC#Cc1cc(-c2c(C)cccc2C)c(F)c([C@@H](CC(=O)O)NC(=O)[C@@H](CC(C)C)n2cc(CCC3CCCN3C)c(C(F)(F)F)cc2=O)c1F] |
| 176 | 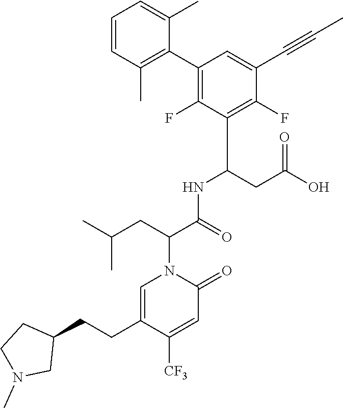 [SMILES: CC#Cc1cc(-c2c(C)cccc2C)c(F)c(C(CC(=O)O)NC(=O)C(CC(C)C)n2cc(CC[C@@H]3CCN(C)C3)c(C(F)(F)F)cc2=O)c1F] |
| | 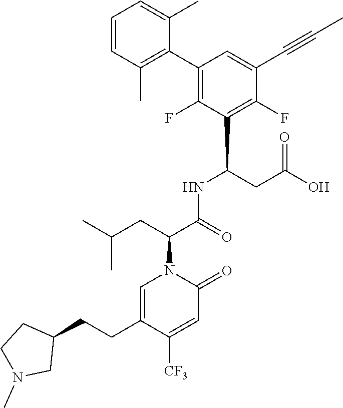 [SMILES: CC#Cc1cc(-c2c(C)cccc2C)c(F)c([C@@H](CC(=O)O)NC(=O)[C@H](CC(C)C)n2cc(CC[C@@H]3CCN(C)C3)c(C(F)(F)F)cc2=O)c1F] |
| | 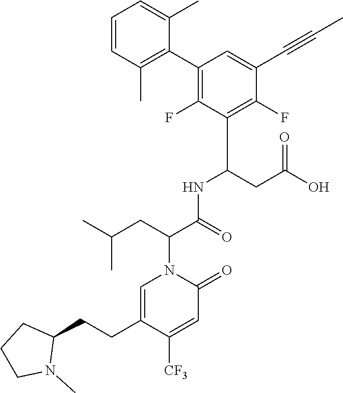 [SMILES: CC#Cc1cc(-c2c(C)cccc2C)c(F)c(C(CC(=O)O)NC(=O)C(CC(C)C)n2cc(CC[C@@H]3CCCN3C)c(C(F)(F)F)cc2=O)c1F] |
| | 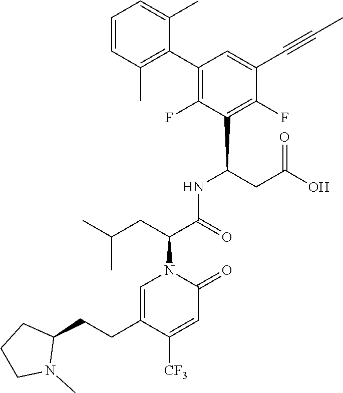 [SMILES: CC#Cc1cc(-c2c(C)cccc2C)c(F)c([C@@H](CC(=O)O)NC(=O)[C@H](CC(C)C)n2cc(CC[C@@H]3CCCN3C)c(C(F)(F)F)cc2=O)c1F] |
| | 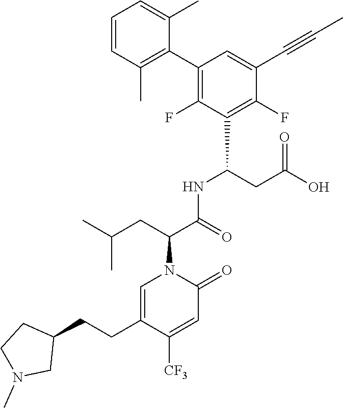 [SMILES: CC#Cc1cc(-c2c(C)cccc2C)c(F)c([C@H](CC(=O)O)NC(=O)[C@H](CC(C)C)n2cc(CC[C@@H]3CCN(C)C3)c(C(F)(F)F)cc2=O)c1F] |
| 177 | 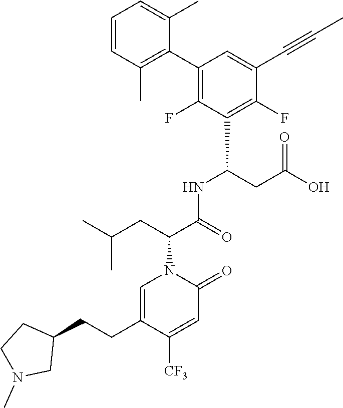 [SMILES: CC#Cc1cc(-c2c(C)cccc2C)c(F)c([C@H](CC(=O)O)NC(=O)[C@@H](CC(C)C)n2cc(CC[C@@H]3CCN(C)C3)c(C(F)(F)F)cc2=O)c1F] |
| | 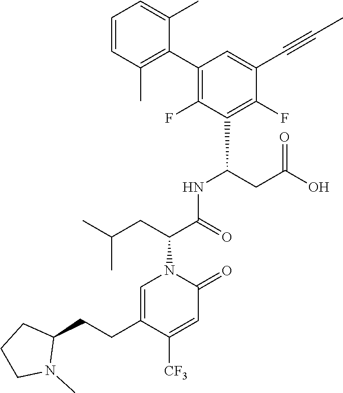 [SMILES: CC#Cc1cc(-c2c(C)cccc2C)c(F)c([C@H](CC(=O)O)NC(=O)[C@@H](CC(C)C)n2cc(CC[C@@H]3CCCN3C)c(C(F)(F)F)cc2=O)c1F] |
| | 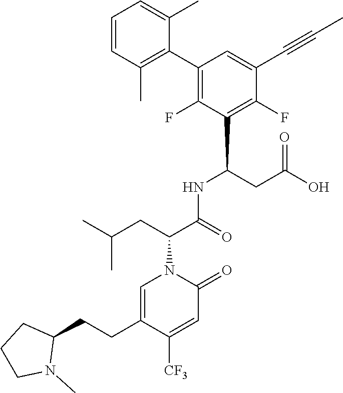 [SMILES: CC#Cc1cc(-c2c(C)cccc2C)c(F)c([C@@H](CC(=O)O)NC(=O)[C@@H](CC(C)C)n2cc(CC[C@@H]3CCCN3C)c(C(F)(F)F)cc2=O)c1F] |
| | 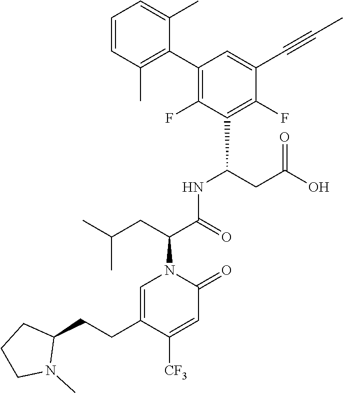 [SMILES: CC#Cc1cc(-c2c(C)cccc2C)c(F)c([C@H](CC(=O)O)NC(=O)[C@H](CC(C)C)n2cc(CC[C@@H]3CCCN3C)c(C(F)(F)F)cc2=O)c1F] |
| | 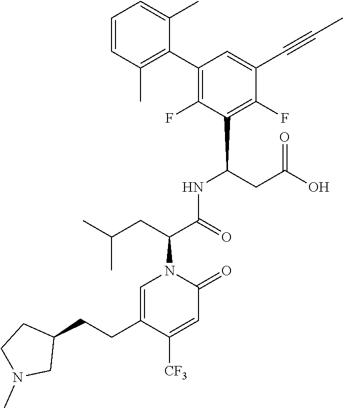 [SMILES: CC#Cc1cc(-c2c(C)cccc2C)c(F)c([C@@H](CC(=O)O)NC(=O)[C@H](CC(C)C)n2cc(CC[C@@H]3CCN(C)C3)c(C(F)(F)F)cc2=O)c1F] |
| 178 | 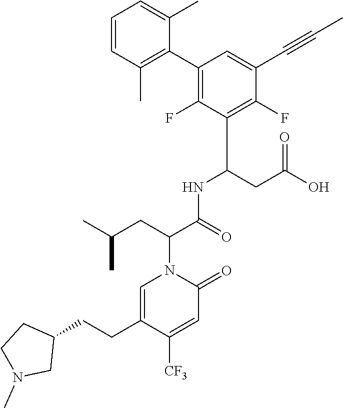 |
| | 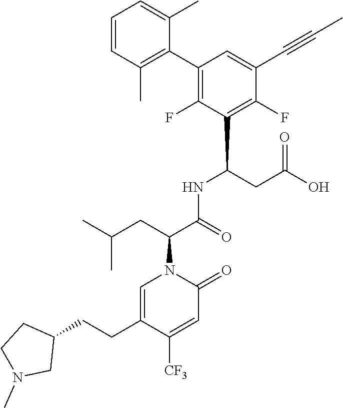 [SMILES: CC#Cc1cc(-c2c(C)cccc2C)c(F)c([C@@H](CC(=O)O)NC(=O)[C@H](CC(C)C)n2cc(CC[C@H]3CCN(C)C3)c(C(F)(F)F)cc2=O)c1F] |
| | 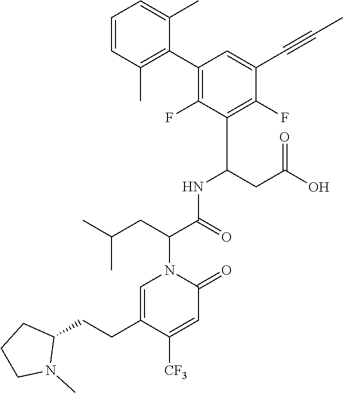 [SMILES: CC#Cc1cc(-c2c(C)cccc2C)c(F)c(C(CC(=O)O)NC(=O)C(CC(C)C)n2cc(CC[C@H]3CCCN3C)c(C(F)(F)F)cc2=O)c1F] |
| | 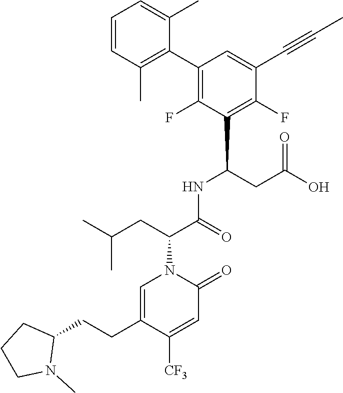 [SMILES: CC#Cc1cc(-c2c(C)cccc2C)c(F)c([C@@H](CC(=O)O)NC(=O)[C@@H](CC(C)C)n2cc(CC[C@H]3CCCN3C)c(C(F)(F)F)cc2=O)c1F] |
| | 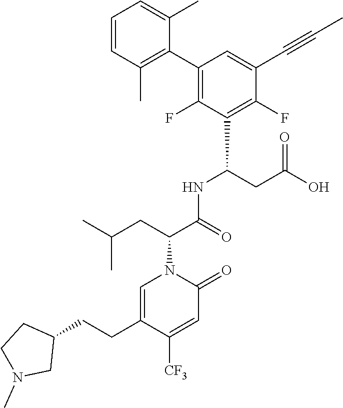 [SMILES: CC#Cc1cc(-c2c(C)cccc2C)c(F)c([C@H](CC(=O)O)NC(=O)[C@@H](CC(C)C)n2cc(CC[C@H]3CCN(C)C3)c(C(F)(F)F)cc2=O)c1F] |
| 179 | 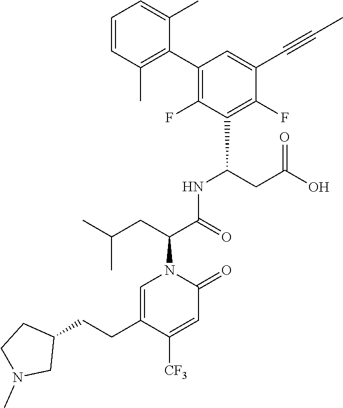 [SMILES: CC#Cc1cc(-c2c(C)cccc2C)c(F)c([C@H](CC(=O)O)NC(=O)[C@H](CC(C)C)n2cc(CC[C@H]3CCN(C)C3)c(C(F)(F)F)cc2=O)c1F] |
| | 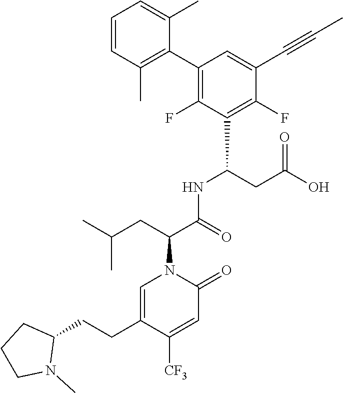 [SMILES: CC#Cc1cc(-c2c(C)cccc2C)c(F)c([C@H](CC(=O)O)NC(=O)[C@H](CC(C)C)n2cc(CC[C@H]3CCCN3C)c(C(F)(F)F)cc2=O)c1F] |
| | 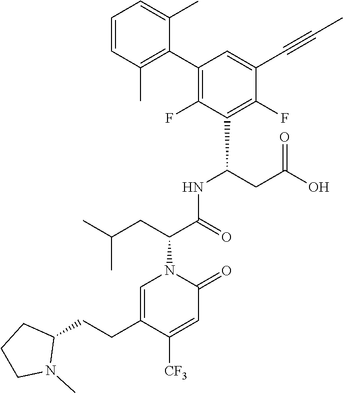 [SMILES: CC#Cc1cc(-c2c(C)cccc2C)c(F)c([C@H](CC(=O)O)NC(=O)[C@@H](CC(C)C)n2cc(CC[C@H]3CCCN3C)c(C(F)(F)F)cc2=O)c1F] |
| | 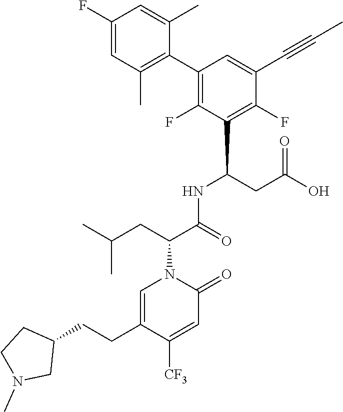 [SMILES: CC#Cc1cc(-c2c(C)cc(F)cc2C)c(F)c([C@@H](CC(=O)O)NC(=O)[C@@H](CC(C)C)n2cc(CC[C@H]3CCN(C)C3)c(C(F)(F)F)cc2=O)c1F] |
| | 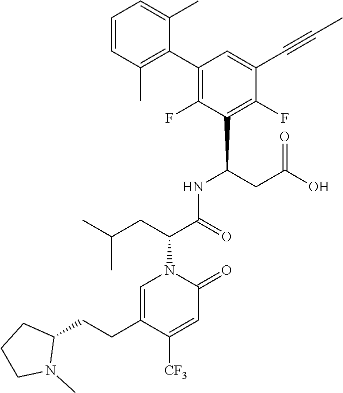 [SMILES: CC#Cc1cc(-c2c(C)cccc2C)c(F)c([C@@H](CC(=O)O)NC(=O)[C@@H](CC(C)C)n2cc(CC[C@H]3CCCN3C)c(C(F)(F)F)cc2=O)c1F] |
| 180 | 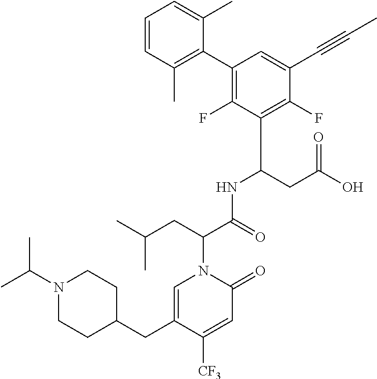 [SMILES: CC#Cc1cc(-c2c(C)cccc2C)c(F)c(C(CC(=O)O)NC(=O)C(CC(C)C)n2cc(CC3CCN(C(C)C)CC3)c(C(F)(F)F)cc2=O)c1F] |
| | 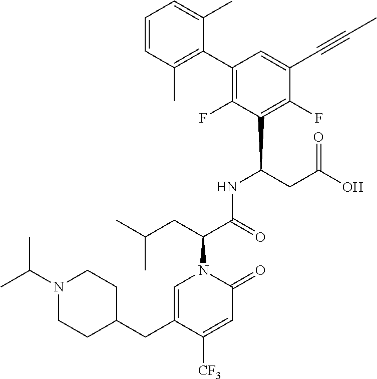 [SMILES: CC#Cc1cc(-c2c(C)cccc2C)c(F)c([C@@H](CC(=O)O)NC(=O)[C@H](CC(C)C)n2cc(CC3CCN(C(C)C)CC3)c(C(F)(F)F)cc2=O)c1F] |
| | 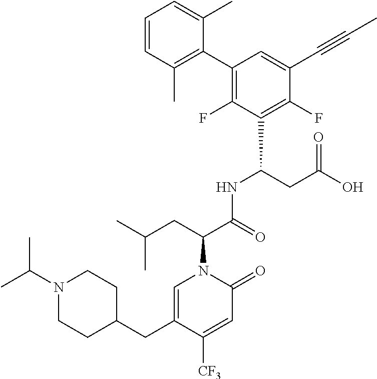 [SMILES: CC#Cc1cc(-c2c(C)cccc2C)c(F)c([C@H](CC(=O)O)NC(=O)[C@H](CC(C)C)n2cc(CC3CCN(C(C)C)CC3)c(C(F)(F)F)cc2=O)c1F] |
| | 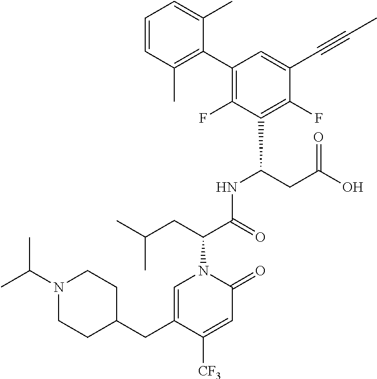 [SMILES: CC#Cc1cc(-c2c(C)cccc2C)c(F)c([C@H](CC(=O)O)NC(=O)[C@@H](CC(C)C)n2cc(CC3CCN(C(C)C)CC3)c(C(F)(F)F)cc2=O)c1F] |
| | 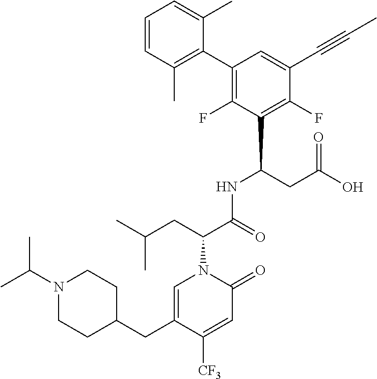 [SMILES: CC#Cc1cc(-c2c(C)cccc2C)c(F)c([C@@H](CC(=O)O)NC(=O)[C@@H](CC(C)C)n2cc(CC3CCN(C(C)C)CC3)c(C(F)(F)F)cc2=O)c1F] |
| 181 | 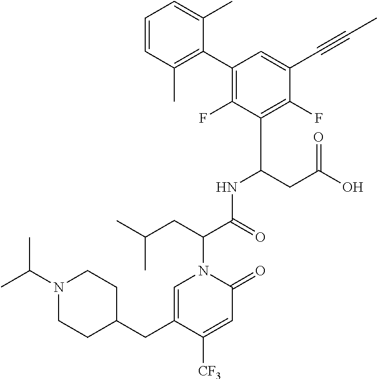 [SMILES: CC#Cc1cc(-c2c(C)cccc2C)c(F)c(C(CC(=O)O)NC(=O)C(CC(C)C)n2cc(CC3CCN(C(C)C)CC3)c(C(F)(F)F)cc2=O)c1F] |
| | 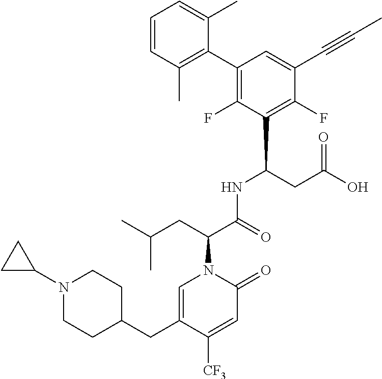 [SMILES: CC#Cc1cc(-c2c(C)cccc2C)c(F)c([C@@H](CC(=O)O)NC(=O)[C@H](CC(C)C)n2cc(CC3CCN(C4CC4)CC3)c(C(F)(F)F)cc2=O)c1F] |
| | 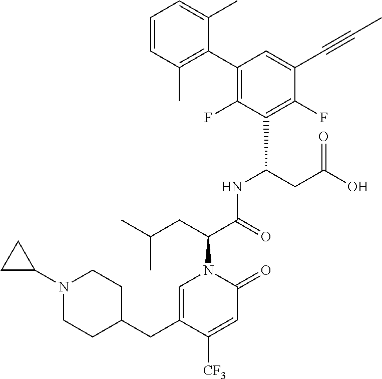 [SMILES: CC#Cc1cc(-c2c(C)cccc2C)c(F)c([C@H](CC(=O)O)NC(=O)[C@H](CC(C)C)n2cc(CC3CCN(C4CC4)CC3)c(C(F)(F)F)cc2=O)c1F] |
| | 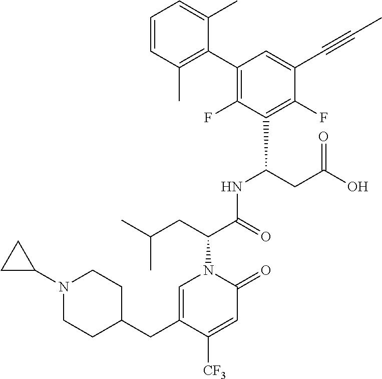 [SMILES: CC#Cc1cc(-c2c(C)cccc2C)c(F)c([C@H](CC(=O)O)NC(=O)[C@@H](CC(C)C)n2cc(CC3CCN(C4CC4)CC3)c(C(F)(F)F)cc2=O)c1F] |
| | 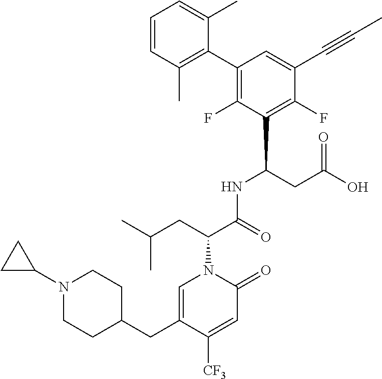 [SMILES: CC#Cc1cc(-c2c(C)cccc2C)c(F)c([C@@H](CC(=O)O)NC(=O)[C@@H](CC(C)C)n2cc(CC3CCN(C4CC4)CC3)c(C(F)(F)F)cc2=O)c1F] |
| 182 | 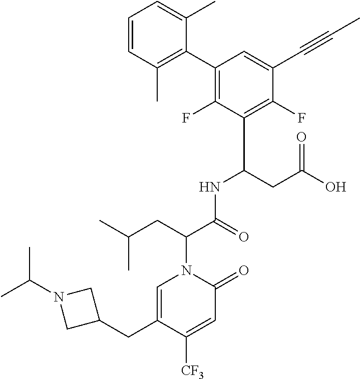 [SMILES: CC#Cc1cc(-c2c(C)cccc2C)c(F)c(C(CC(=O)O)NC(=O)C(CC(C)C)n2cc(CC3CN(C(C)C)C3)c(C(F)(F)F)cc2=O)c1F] |
| | 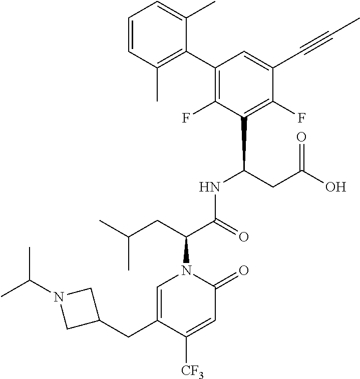 [SMILES: CC#Cc1cc(-c2c(C)cccc2C)c(F)c([C@@H](CC(=O)O)NC(=O)[C@H](CC(C)C)n2cc(CC3CN(C(C)C)C3)c(C(F)(F)F)cc2=O)c1F] |
| | 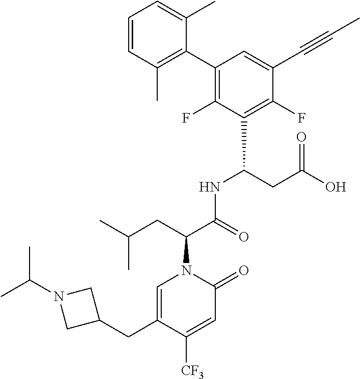 [SMILES: CC#Cc1cc(-c2c(C)cccc2C)c(F)c([C@H](CC(=O)O)NC(=O)[C@H](CC(C)C)n2cc(CC3CN(C(C)C)C3)c(C(F)(F)F)cc2=O)c1F] |
| | 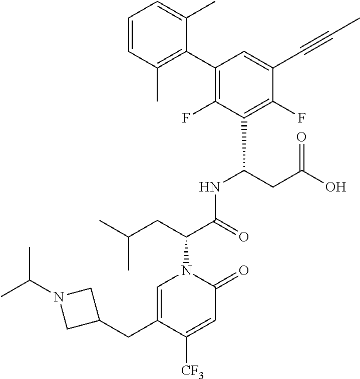 [SMILES: CC#Cc1cc(-c2c(C)cccc2C)c(F)c([C@H](CC(=O)O)NC(=O)[C@@H](CC(C)C)n2cc(CC3CN(C(C)C)C3)c(C(F)(F)F)cc2=O)c1F] |
| | 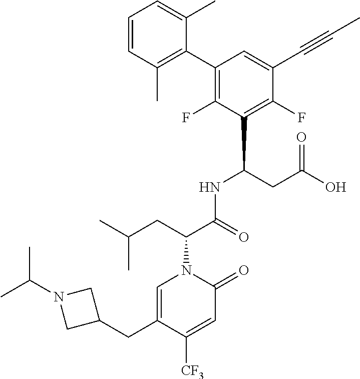 [SMILES: CC#Cc1cc(-c2c(C)cccc2C)c(F)c([C@@H](CC(=O)O)NC(=O)[C@@H](CC(C)C)n2cc(CC3CN(C(C)C)C3)c(C(F)(F)F)cc2=O)c1F] |
| 183 | 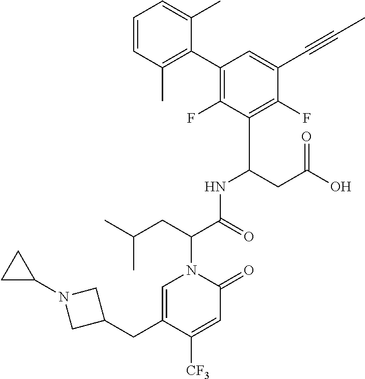 [SMILES: CC#Cc1cc(-c2c(C)cccc2C)c(F)c(C(CC(=O)O)NC(=O)C(CC(C)C)n2cc(CC3CN(C4CC4)C3)c(C(F)(F)F)cc2=O)c1F] |
| | 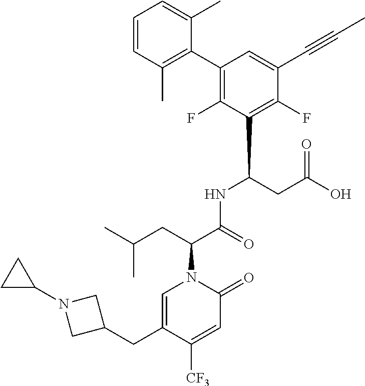 [SMILES: CC#Cc1cc(-c2c(C)cccc2C)c(F)c([C@@H](CC(=O)O)NC(=O)[C@H](CC(C)C)n2cc(CC3CN(C4CC4)C3)c(C(F)(F)F)cc2=O)c1F] |
| | 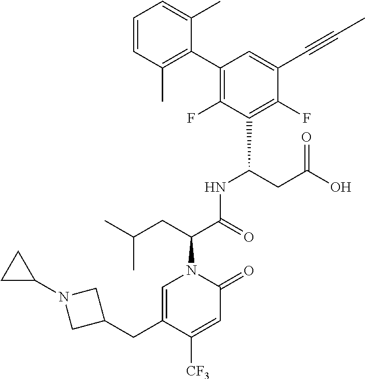 [SMILES: CC#Cc1cc(-c2c(C)cccc2C)c(F)c([C@H](CC(=O)O)NC(=O)[C@H](CC(C)C)n2cc(CC3CN(C4CC4)C3)c(C(F)(F)F)cc2=O)c1F] |
| | 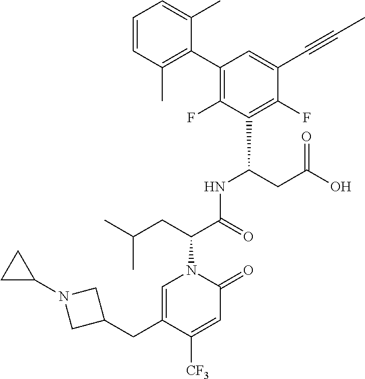 [SMILES: CC#Cc1cc(-c2c(C)cccc2C)c(F)c([C@H](CC(=O)O)NC(=O)[C@@H](CC(C)C)n2cc(CC3CN(C4CC4)C3)c(C(F)(F)F)cc2=O)c1F] |
| | 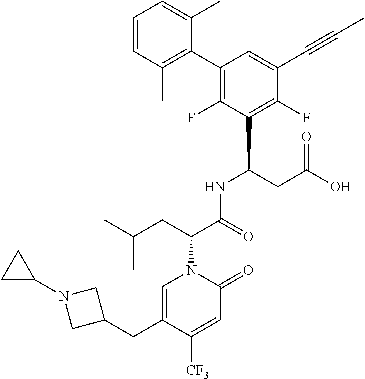 [SMILES: CC#Cc1cc(-c2c(C)cccc2C)c(F)c([C@@H](CC(=O)O)NC(=O)[C@@H](CC(C)C)n2cc(CC3CN(C4CC4)C3)c(C(F)(F)F)cc2=O)c1F] |
| 184 | 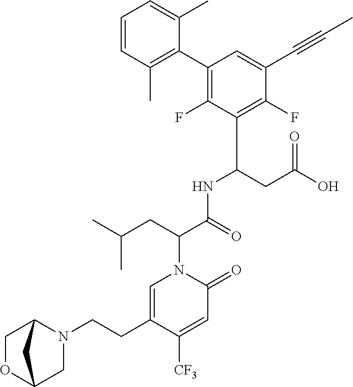 [SMILES: CC#Cc1cc(-c2c(C)cccc2C)c(F)c(C(CC(=O)O)NC(=O)C(CC(C)C)n2cc(CCN3C[C@H]4C[C@@H]3CO4)c(C(F)(F)F)cc2=O)c1F] |
| | 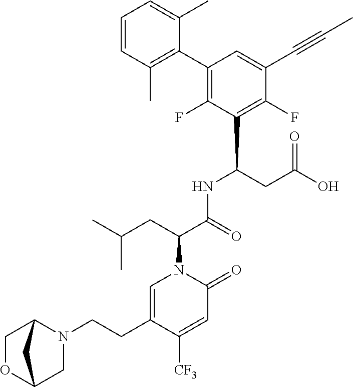 [SMILES: CC#Cc1cc(-c2c(C)cccc2C)c(F)c([C@@H](CC(=O)O)NC(=O)[C@H](CC(C)C)n2cc(CCN3C[C@H]4C[C@@H]3CO4)c(C(F)(F)F)cc2=O)c1F] |
| | 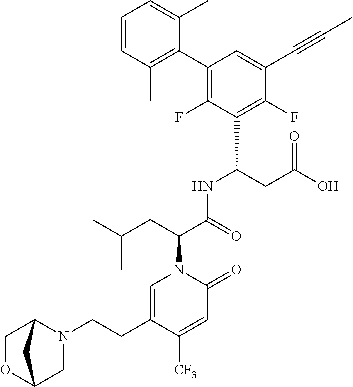 [SMILES: CC#Cc1cc(-c2c(C)cccc2C)c(F)c([C@H](CC(=O)O)NC(=O)[C@H](CC(C)C)n2cc(CCN3C[C@H]4C[C@@H]3CO4)c(C(F)(F)F)cc2=O)c1F] |
| | 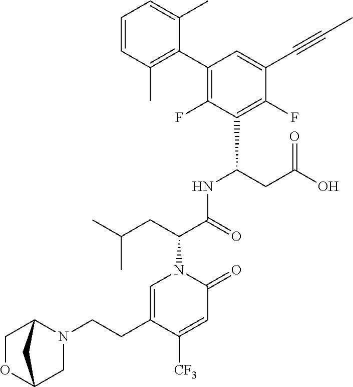 [SMILES: CC#Cc1cc(-c2c(C)cccc2C)c(F)c([C@H](CC(=O)O)NC(=O)[C@@H](CC(C)C)n2cc(CCN3C[C@H]4C[C@@H]3CO4)c(C(F)(F)F)cc2=O)c1F] |
| | 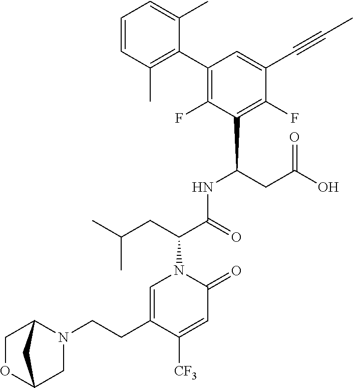 [SMILES: CC#Cc1cc(-c2c(C)cccc2C)c(F)c([C@@H](CC(=O)O)NC(=O)[C@@H](CC(C)C)n2cc(CCN3C[C@H]4C[C@@H]3CO4)c(C(F)(F)F)cc2=O)c1F] |
| 185 | 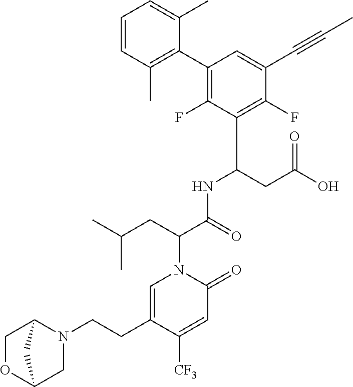 [SMILES: CC#Cc1cc(-c2c(C)cccc2C)c(F)c(C(CC(=O)O)NC(=O)C(CC(C)C)n2cc(CCN3C[C@@H]4C[C@H]3CO4)c(C(F)(F)F)cc2=O)c1F] |
| | 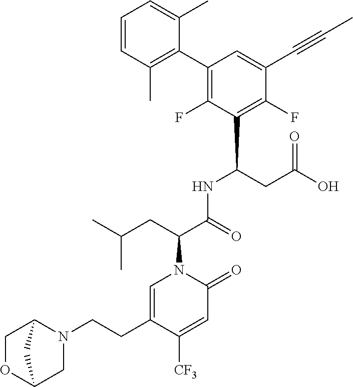 [SMILES: CC#Cc1cc(-c2c(C)cccc2C)c(F)c([C@@H](CC(=O)O)NC(=O)[C@H](CC(C)C)n2cc(CCN3C[C@@H]4C[C@H]3CO4)c(C(F)(F)F)cc2=O)c1F] |
| | 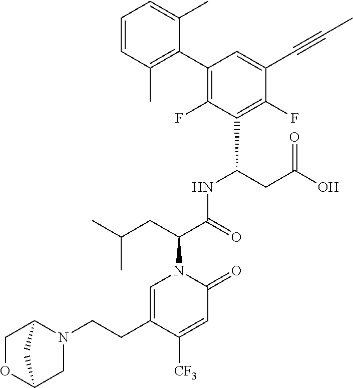 [SMILES: CC#Cc1cc(-c2c(C)cccc2C)c(F)c([C@H](CC(=O)O)NC(=O)[C@H](CC(C)C)n2cc(CCN3C[C@@H]4C[C@H]3CO4)c(C(F)(F)F)cc2=O)c1F] |
| | 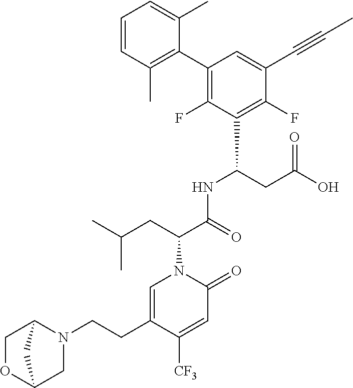 [SMILES: CC#Cc1cc(-c2c(C)cccc2C)c(F)c([C@H](CC(=O)O)NC(=O)[C@@H](CC(C)C)n2cc(CCN3C[C@@H]4C[C@H]3CO4)c(C(F)(F)F)cc2=O)c1F] |
| | 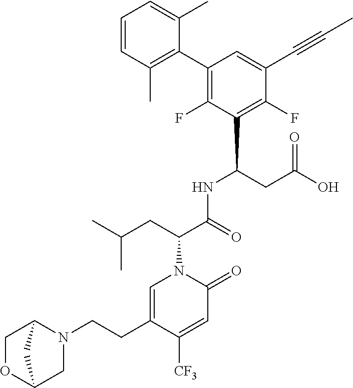 [SMILES: CC#Cc1cc(-c2c(C)cccc2C)c(F)c([C@@H](CC(=O)O)NC(=O)[C@@H](CC(C)C)n2cc(CCN3C[C@@H]4C[C@H]3CO4)c(C(F)(F)F)cc2=O)c1F] |
| 186 | 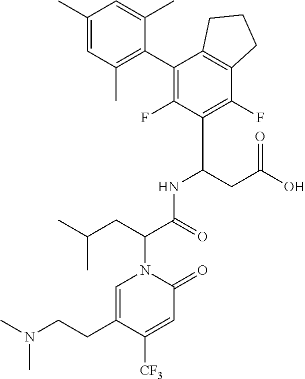 [SMILES: Cc1cc(C)c(-c2c(F)c(C(CC(=O)O)NC(=O)C(CC(C)C)n3cc(CCN(C)C)c(C(F)(F)F)cc3=O)c(F)c3c2CCC3)c(C)c1] |
| | 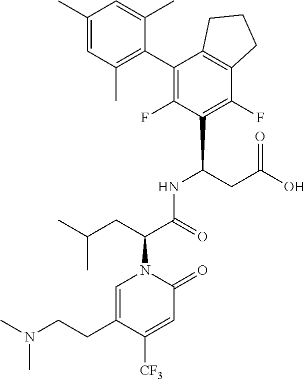 [SMILES: Cc1cc(C)c(-c2c(F)c([C@@H](CC(=O)O)NC(=O)[C@H](CC(C)C)n3cc(CCN(C)C)c(C(F)(F)F)cc3=O)c(F)c3c2CCC3)c(C)c1] |
| | 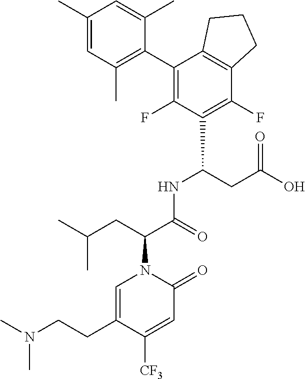 [SMILES: Cc1cc(C)c(-c2c(F)c([C@H](CC(=O)O)NC(=O)[C@H](CC(C)C)n3cc(CCN(C)C)c(C(F)(F)F)cc3=O)c(F)c3c2CCC3)c(C)c1] |
| | 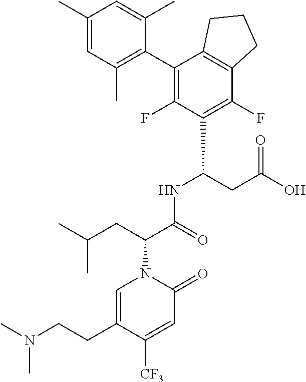 [SMILES: Cc1cc(C)c(-c2c(F)c([C@H](CC(=O)O)NC(=O)[C@@H](CC(C)C)n3cc(CCN(C)C)c(C(F)(F)F)cc3=O)c(F)c3c2CCC3)c(C)c1] |
| | 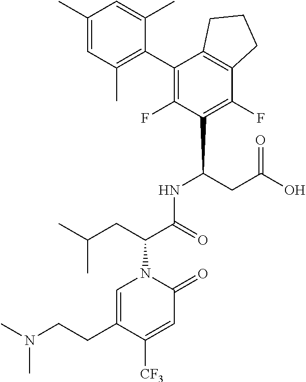 [SMILES: Cc1cc(C)c(-c2c(F)c([C@@H](CC(=O)O)NC(=O)[C@@H](CC(C)C)n3cc(CCN(C)C)c(C(F)(F)F)cc3=O)c(F)c3c2CCC3)c(C)c1] |
| 187 | 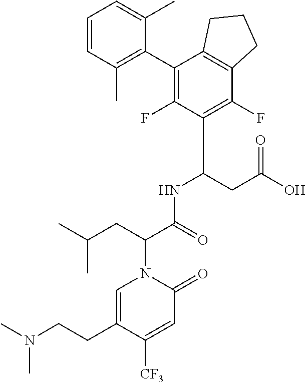 [SMILES: Cc1cccc(C)c1-c1c(F)c(C(CC(=O)O)NC(=O)C(CC(C)C)n2cc(CCN(C)C)c(C(F)(F)F)cc2=O)c(F)c2c1CCC2] |
| | 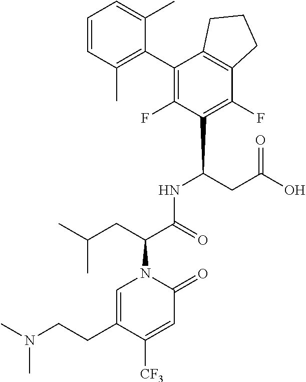 [SMILES: Cc1cccc(C)c1-c1c(F)c([C@@H](CC(=O)O)NC(=O)[C@H](CC(C)C)n2cc(CCN(C)C)c(C(F)(F)F)cc2=O)c(F)c2c1CCC2] |
| | 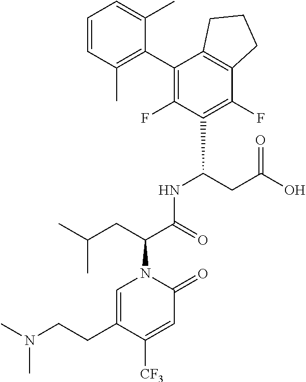 [SMILES: Cc1cccc(C)c1-c1c(F)c([C@H](CC(=O)O)NC(=O)[C@H](CC(C)C)n2cc(CCN(C)C)c(C(F)(F)F)cc2=O)c(F)c2c1CCC2] |
| | 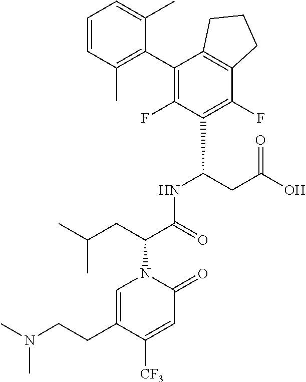 [SMILES: Cc1cccc(C)c1-c1c(F)c([C@H](CC(=O)O)NC(=O)[C@@H](CC(C)C)n2cc(CCN(C)C)c(C(F)(F)F)cc2=O)c(F)c2c1CCC2] |
| | 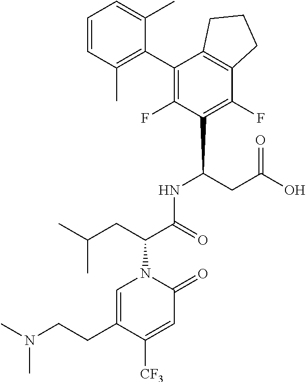 [SMILES: Cc1cccc(C)c1-c1c(F)c([C@@H](CC(=O)O)NC(=O)[C@@H](CC(C)C)n2cc(CCN(C)C)c(C(F)(F)F)cc2=O)c(F)c2c1CCC2] |
| 188 | 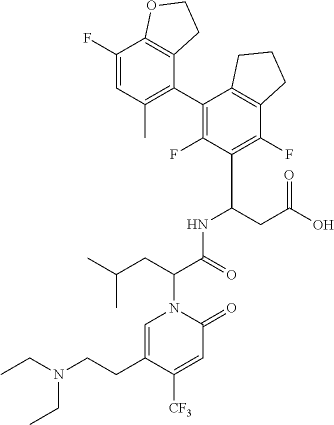 [SMILES: CCN(CC)CCc1cn(C(CC(C)C)C(=O)NC(CC(=O)O)c2c(F)c3c(c(-c4c(C)cc(F)c5c4CCO5)c2F)CCC3)c(=O)cc1C(F)(F)F] |
| | 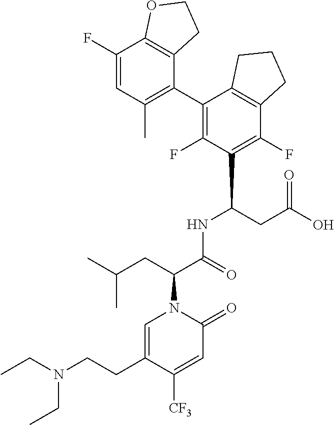 [SMILES: CCN(CC)CCc1cn([C@@H](CC(C)C)C(=O)N[C@H](CC(=O)O)c2c(F)c3c(c(-c4c(C)cc(F)c5c4CCO5)c2F)CCC3)c(=O)cc1C(F)(F)F] |
| | 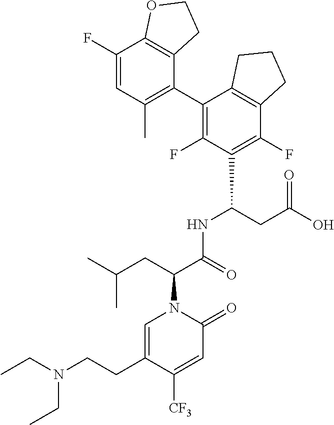 [SMILES: CCN(CC)CCc1cn([C@@H](CC(C)C)C(=O)N[C@@H](CC(=O)O)c2c(F)c3c(c(-c4c(C)cc(F)c5c4CCO5)c2F)CCC3)c(=O)cc1C(F)(F)F] |
| | 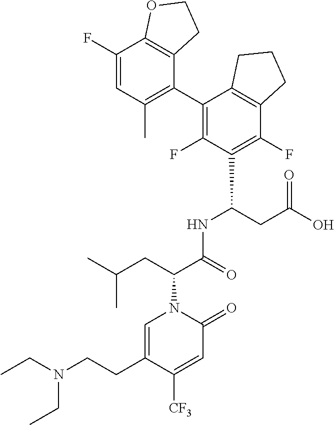 [SMILES: CCN(CC)CCc1cn([C@H](CC(C)C)C(=O)N[C@@H](CC(=O)O)c2c(F)c3c(c(-c4c(C)cc(F)c5c4CCO5)c2F)CCC3)c(=O)cc1C(F)(F)F] |
| | 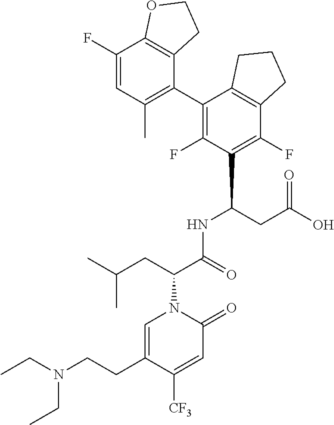 [SMILES: CCN(CC)CCc1cn([C@H](CC(C)C)C(=O)N[C@H](CC(=O)O)c2c(F)c3c(c(-c4c(C)cc(F)c5c4CCO5)c2F)CCC3)c(=O)cc1C(F)(F)F] |
| 189 | 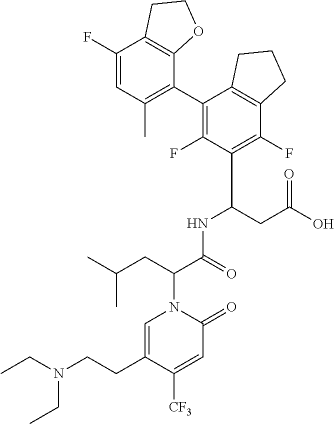 [SMILES: CCN(CC)CCc1cn(C(CC(C)C)C(=O)NC(CC(=O)O)c2c(F)c3c(c(-c4c(C)cc(F)c5c4OCC5)c2F)CCC3)c(=O)cc1C(F)(F)F] |
| | 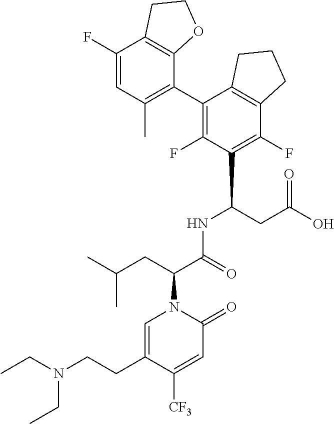 [SMILES: CCN(CC)CCc1cn([C@@H](CC(C)C)C(=O)N[C@H](CC(=O)O)c2c(F)c3c(c(-c4c(C)cc(F)c5c4OCC5)c2F)CCC3)c(=O)cc1C(F)(F)F] |
| | 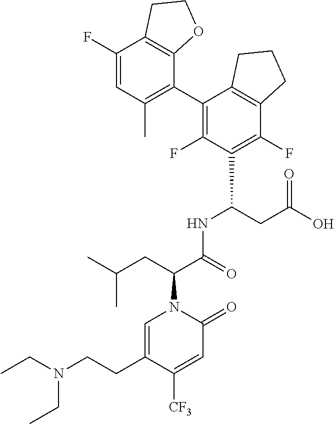 [SMILES: CCN(CC)CCc1cn([C@@H](CC(C)C)C(=O)N[C@@H](CC(=O)O)c2c(F)c3c(c(-c4c(C)cc(F)c5c4OCC5)c2F)CCC3)c(=O)cc1C(F)(F)F] |
| | 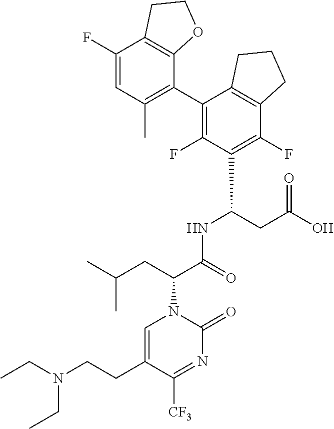 [SMILES: CCN(CC)CCc1cn([C@H](CC(C)C)C(=O)N[C@@H](CC(=O)O)c2c(F)c3c(c(-c4c(C)cc(F)c5c4OCC5)c2F)CCC3)c(=O)nc1C(F)(F)F] |
| | 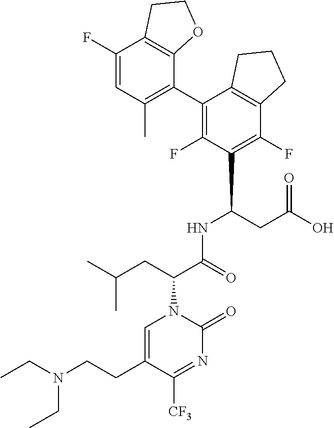 [SMILES: CCN(CC)CCc1cn([C@H](CC(C)C)C(=O)N[C@H](CC(=O)O)c2c(F)c3c(c(-c4c(C)cc(F)c5c4OCC5)c2F)CCC3)c(=O)nc1C(F)(F)F] |
| 190 | 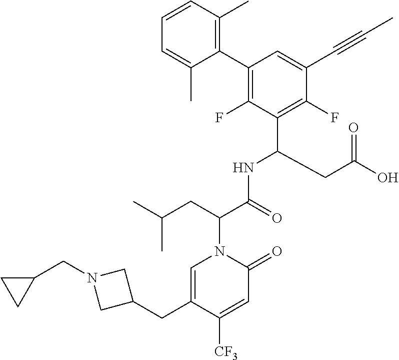 [SMILES: CC#Cc1cc(-c2c(C)cccc2C)c(F)c(C(CC(=O)O)NC(=O)C(CC(C)C)n2cc(CC3CN(CC4CC4)C3)c(C(F)(F)F)cc2=O)c1F] |
| | 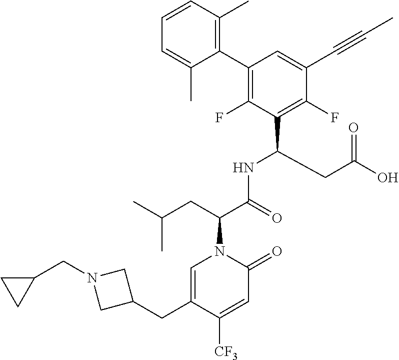 [SMILES: CC#Cc1cc(-c2c(C)cccc2C)c(F)c([C@@H](CC(=O)O)NC(=O)[C@H](CC(C)C)n2cc(CC3CN(CC4CC4)C3)c(C(F)(F)F)cc2=O)c1F] |
| | 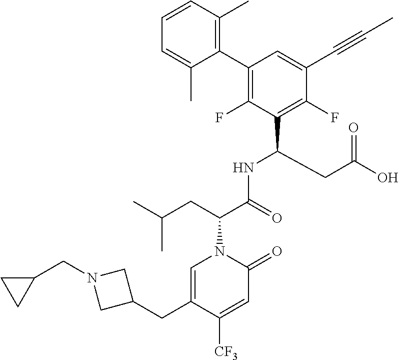 [SMILES: CC#Cc1cc(-c2c(C)cccc2C)c(F)c([C@@H](CC(=O)O)NC(=O)[C@@H](CC(C)C)n2cc(CC3CN(CC4CC4)C3)c(C(F)(F)F)cc2=O)c1F] |
| | 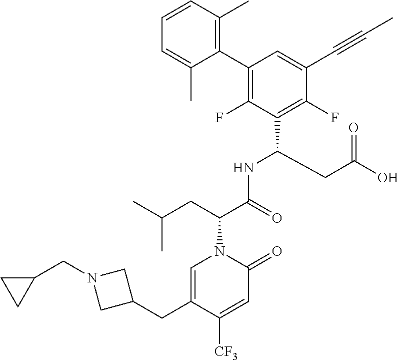 [SMILES: CC#Cc1cc(-c2c(C)cccc2C)c(F)c([C@H](CC(=O)O)NC(=O)[C@@H](CC(C)C)n2cc(CC3CN(CC4CC4)C3)c(C(F)(F)F)cc2=O)c1F] |
| | 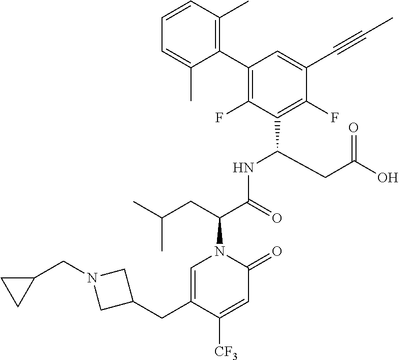 [SMILES: CC#Cc1cc(-c2c(C)cccc2C)c(F)c([C@H](CC(=O)O)NC(=O)[C@H](CC(C)C)n2cc(CC3CN(CC4CC4)C3)c(C(F)(F)F)cc2=O)c1F] |
| 191 | 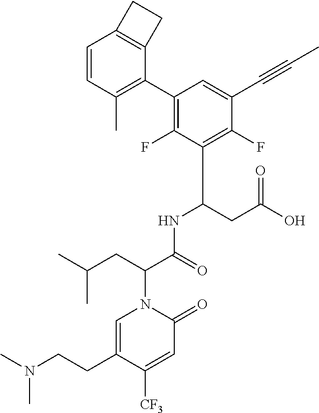 [SMILES: CC#Cc1cc(-c2c(C)ccc3c2CC3)c(F)c(C(CC(=O)O)NC(=O)C(CC(C)C)n2cc(CCN(C)C)c(C(F)(F)F)cc2=O)c1F] |
| | 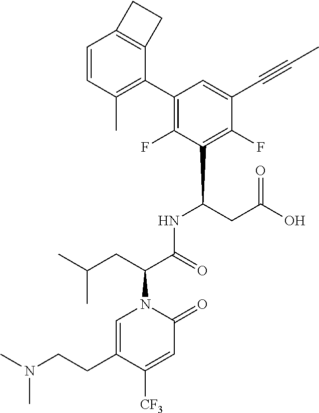 [SMILES: CC#Cc1cc(-c2c(C)ccc3c2CC3)c(F)c([C@@H](CC(=O)O)NC(=O)[C@H](CC(C)C)n2cc(CCN(C)C)c(C(F)(F)F)cc2=O)c1F] |
| | 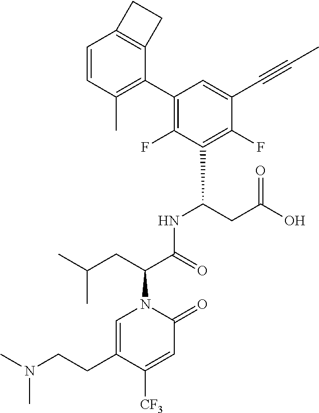 [SMILES: CC#Cc1cc(-c2c(C)ccc3c2CC3)c(F)c([C@H](CC(=O)O)NC(=O)[C@H](CC(C)C)n2cc(CCN(C)C)c(C(F)(F)F)cc2=O)c1F] |
| | 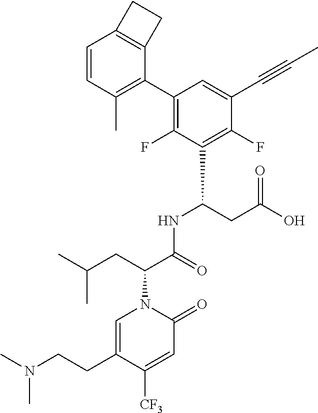 [SMILES: CC#Cc1cc(-c2c(C)ccc3c2CC3)c(F)c([C@H](CC(=O)O)NC(=O)[C@@H](CC(C)C)n2cc(CCN(C)C)c(C(F)(F)F)cc2=O)c1F] |
| | 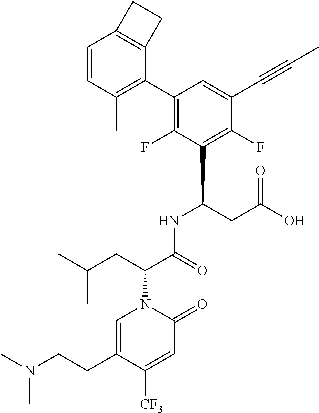 [SMILES: CC#Cc1cc(-c2c(C)ccc3c2CC3)c(F)c([C@@H](CC(=O)O)NC(=O)[C@@H](CC(C)C)n2cc(CCN(C)C)c(C(F)(F)F)cc2=O)c1F] |
| 192 | 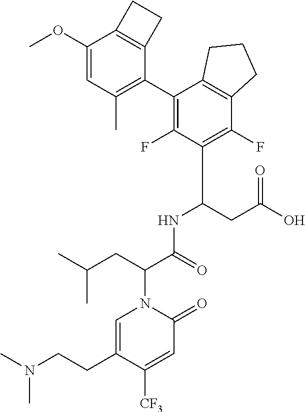 [SMILES: COc1cc(C)c(-c2c(F)c(C(CC(=O)O)NC(=O)C(CC(C)C)n3cc(CCN(C)C)c(C(F)(F)F)cc3=O)c(F)c3c2CCC3)c2c1CC2] |
| | 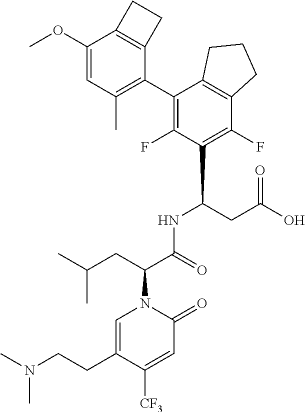 [SMILES: COc1cc(C)c(-c2c(F)c([C@@H](CC(=O)O)NC(=O)[C@H](CC(C)C)n3cc(CCN(C)C)c(C(F)(F)F)cc3=O)c(F)c3c2CCC3)c2c1CC2] |
| | 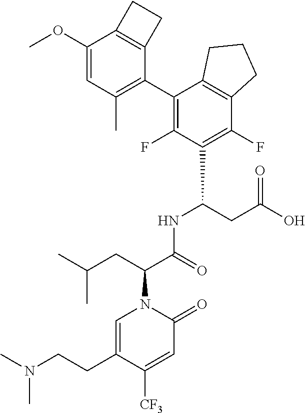 [SMILES: COc1cc(C)c(-c2c(F)c([C@H](CC(=O)O)NC(=O)[C@H](CC(C)C)n3cc(CCN(C)C)c(C(F)(F)F)cc3=O)c(F)c3c2CCC3)c2c1CC2] |
| | 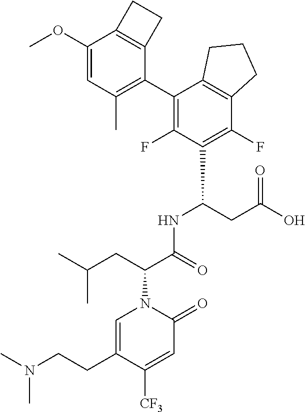 [SMILES: COc1cc(C)c(-c2c(F)c([C@H](CC(=O)O)NC(=O)[C@@H](CC(C)C)n3cc(CCN(C)C)c(C(F)(F)F)cc3=O)c(F)c3c2CCC3)c2c1CC2] |
| | 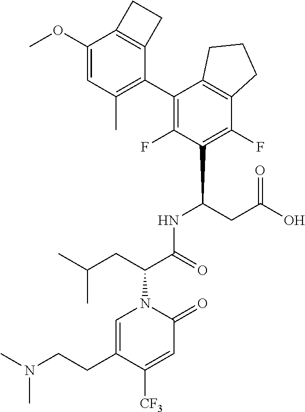 [SMILES: COc1cc(C)c(-c2c(F)c([C@@H](CC(=O)O)NC(=O)[C@@H](CC(C)C)n3cc(CCN(C)C)c(C(F)(F)F)cc3=O)c(F)c3c2CCC3)c2c1CC2] |
| 193 | 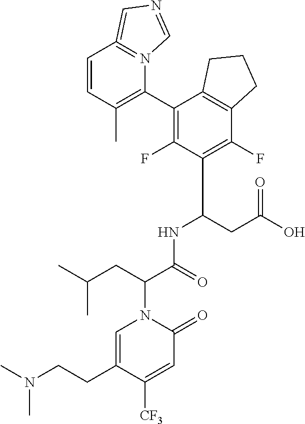 |
| | 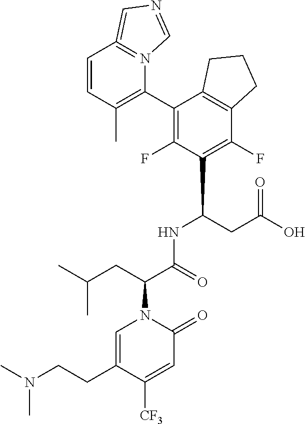 |
| | 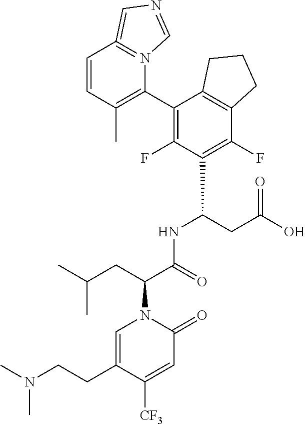 |
| | 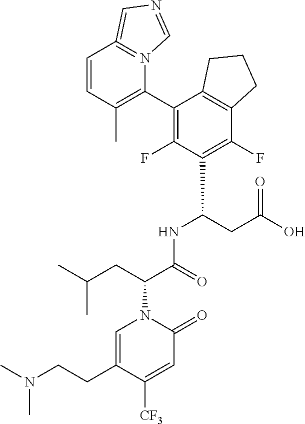 |
| | 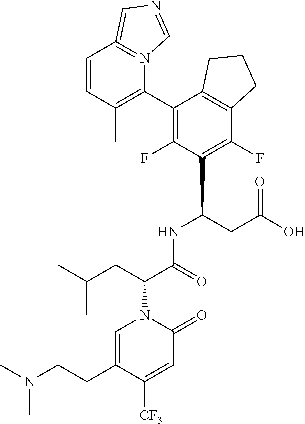 |
| 194 | 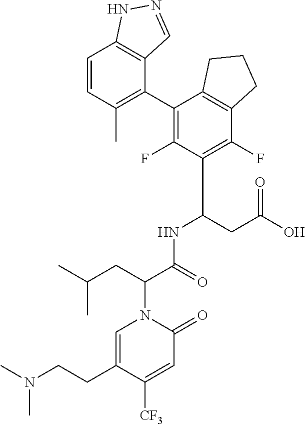 [SMILES: Cc1ccc2[nH]ncc2c1-c1c(F)c(C(CC(=O)O)NC(=O)C(CC(C)C)n2cc(CCN(C)C)c(C(F)(F)F)cc2=O)c(F)c2c1CCC2] |
| | 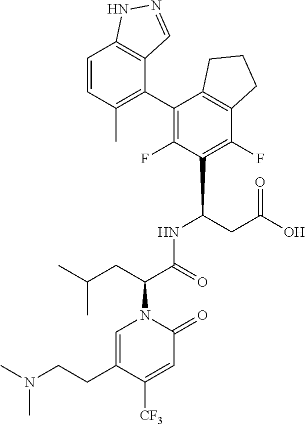 [SMILES: Cc1ccc2[nH]ncc2c1-c1c(F)c([C@@H](CC(=O)O)NC(=O)[C@H](CC(C)C)n2cc(CCN(C)C)c(C(F)(F)F)cc2=O)c(F)c2c1CCC2] |
| | 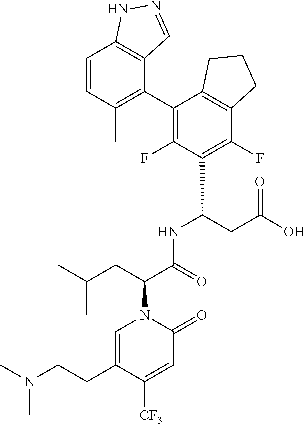 [SMILES: Cc1ccc2[nH]ncc2c1-c1c(F)c([C@H](CC(=O)O)NC(=O)[C@H](CC(C)C)n2cc(CCN(C)C)c(C(F)(F)F)cc2=O)c(F)c2c1CCC2] |
| | 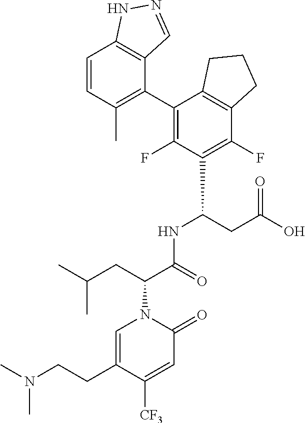 [SMILES: Cc1ccc2[nH]ncc2c1-c1c(F)c([C@H](CC(=O)O)NC(=O)[C@@H](CC(C)C)n2cc(CCN(C)C)c(C(F)(F)F)cc2=O)c(F)c2c1CCC2] |
| | 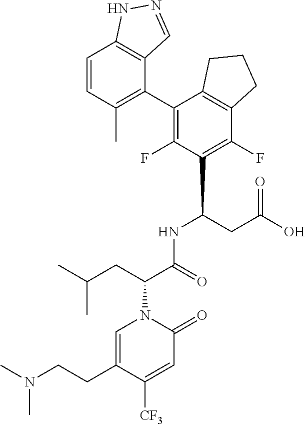 [SMILES: Cc1ccc2[nH]ncc2c1-c1c(F)c([C@@H](CC(=O)O)NC(=O)[C@@H](CC(C)C)n2cc(CCN(C)C)c(C(F)(F)F)cc2=O)c(F)c2c1CCC2] |
| 195 | 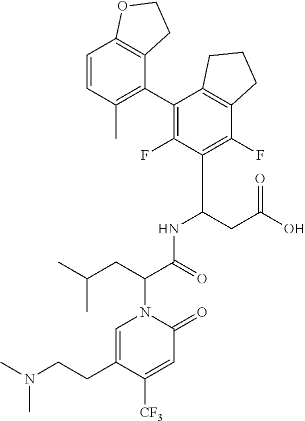 [SMILES: Cc1ccc2c(c1-c1c(F)c(C(CC(=O)O)NC(=O)C(CC(C)C)n3cc(CCN(C)C)c(C(F)(F)F)cc3=O)c(F)c3c1CCC3)CCO2] |
| | 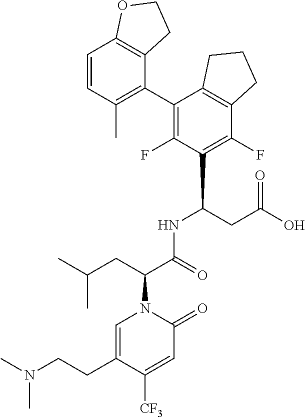 [SMILES: Cc1ccc2c(c1-c1c(F)c([C@@H](CC(=O)O)NC(=O)[C@H](CC(C)C)n3cc(CCN(C)C)c(C(F)(F)F)cc3=O)c(F)c3c1CCC3)CCO2] |
| | 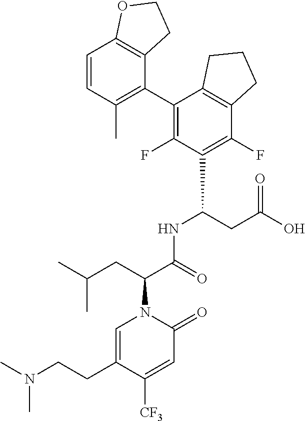 [SMILES: Cc1ccc2c(c1-c1c(F)c([C@H](CC(=O)O)NC(=O)[C@H](CC(C)C)n3cc(CCN(C)C)c(C(F)(F)F)cc3=O)c(F)c3c1CCC3)CCO2] |
| | 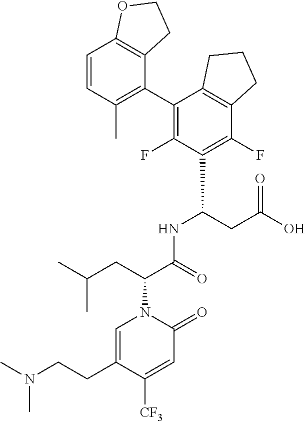 [SMILES: Cc1ccc2c(c1-c1c(F)c([C@H](CC(=O)O)NC(=O)[C@@H](CC(C)C)n3cc(CCN(C)C)c(C(F)(F)F)cc3=O)c(F)c3c1CCC3)CCO2] |
| | 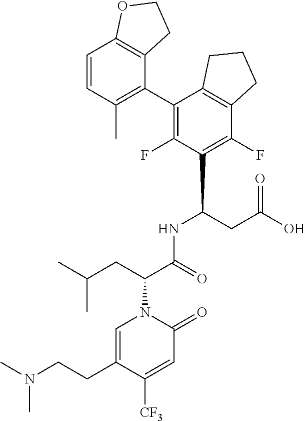 [SMILES: Cc1ccc2c(c1-c1c(F)c([C@@H](CC(=O)O)NC(=O)[C@@H](CC(C)C)n3cc(CCN(C)C)c(C(F)(F)F)cc3=O)c(F)c3c1CCC3)CCO2] |
| 196 | 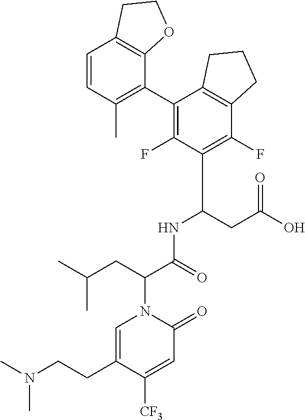 [SMILES: Cc1ccc2c(c1-c1c(F)c(C(CC(=O)O)NC(=O)C(CC(C)C)n3cc(CCN(C)C)c(C(F)(F)F)cc3=O)c(F)c3c1CCC3)OCC2] |
| | 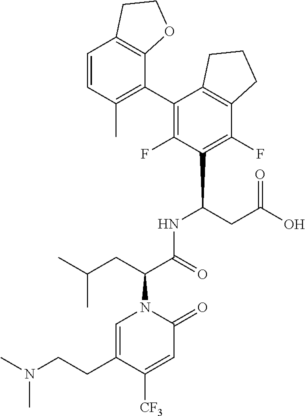 [SMILES: Cc1ccc2c(c1-c1c(F)c([C@@H](CC(=O)O)NC(=O)[C@H](CC(C)C)n3cc(CCN(C)C)c(C(F)(F)F)cc3=O)c(F)c3c1CCC3)OCC2] |
| | 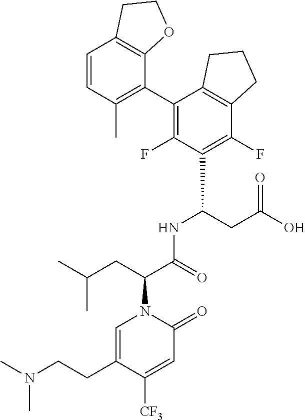 [SMILES: Cc1ccc2c(c1-c1c(F)c([C@H](CC(=O)O)NC(=O)[C@H](CC(C)C)n3cc(CCN(C)C)c(C(F)(F)F)cc3=O)c(F)c3c1CCC3)OCC2] |
| | 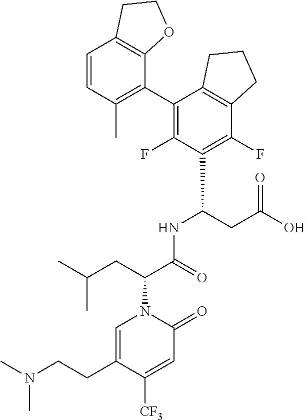 [SMILES: Cc1ccc2c(c1-c1c(F)c([C@H](CC(=O)O)NC(=O)[C@@H](CC(C)C)n3cc(CCN(C)C)c(C(F)(F)F)cc3=O)c(F)c3c1CCC3)OCC2] |
| | 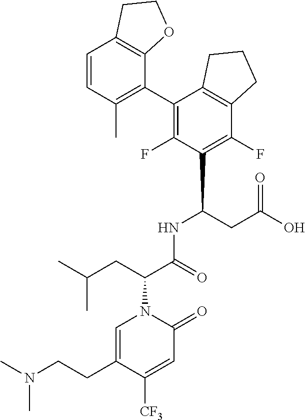 [SMILES: Cc1ccc2c(c1-c1c(F)c([C@@H](CC(=O)O)NC(=O)[C@@H](CC(C)C)n3cc(CCN(C)C)c(C(F)(F)F)cc3=O)c(F)c3c1CCC3)OCC2] |
| 197 | 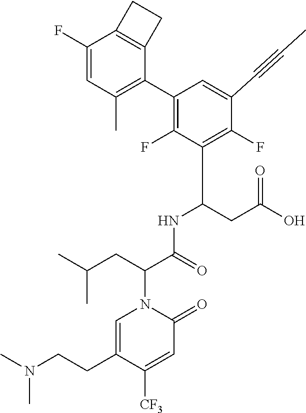 [SMILES: CC#Cc1cc(-c2c(C)cc(F)c3c2CC3)c(F)c(C(CC(=O)O)NC(=O)C(CC(C)C)n2cc(CCN(C)C)c(C(F)(F)F)cc2=O)c1F] |
| | 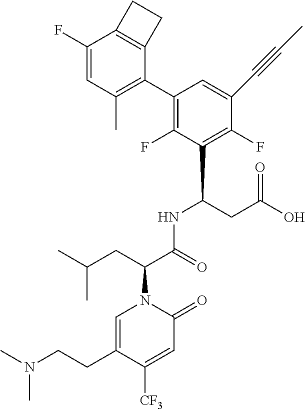 [SMILES: CC#Cc1cc(-c2c(C)cc(F)c3c2CC3)c(F)c([C@@H](CC(=O)O)NC(=O)[C@H](CC(C)C)n2cc(CCN(C)C)c(C(F)(F)F)cc2=O)c1F] |
| | 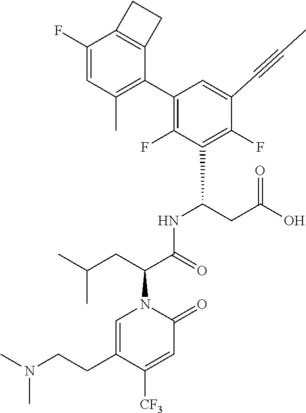 [SMILES: CC#Cc1cc(-c2c(C)cc(F)c3c2CC3)c(F)c([C@H](CC(=O)O)NC(=O)[C@H](CC(C)C)n2cc(CCN(C)C)c(C(F)(F)F)cc2=O)c1F] |
| | 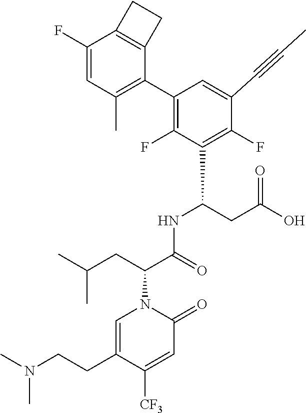 [SMILES: CC#Cc1cc(-c2c(C)cc(F)c3c2CC3)c(F)c([C@H](CC(=O)O)NC(=O)[C@@H](CC(C)C)n2cc(CCN(C)C)c(C(F)(F)F)cc2=O)c1F] |
| | 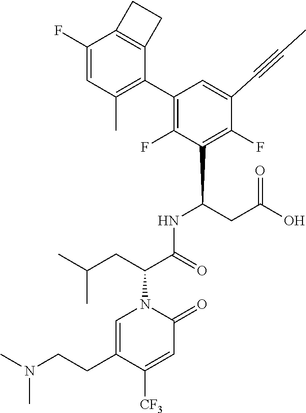 [SMILES: CC#Cc1cc(-c2c(C)cc(F)c3c2CC3)c(F)c([C@@H](CC(=O)O)NC(=O)[C@@H](CC(C)C)n2cc(CCN(C)C)c(C(F)(F)F)cc2=O)c1F] |
| 198 | 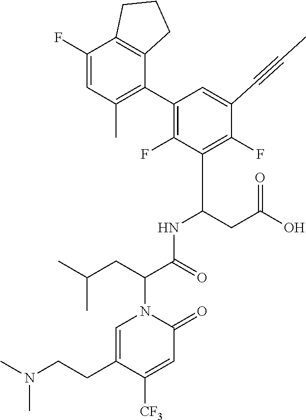 [SMILES: CC#Cc1cc(-c2c(C)cc(F)c3c2CCC3)c(F)c(C(CC(=O)O)NC(=O)C(CC(C)C)n2cc(CCN(C)C)c(C(F)(F)F)cc2=O)c1F] |
| | 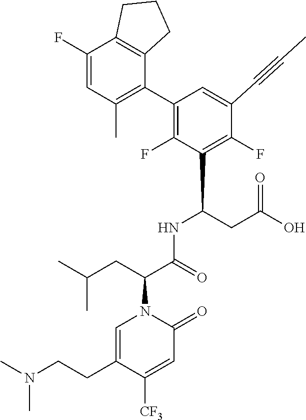 [SMILES: CC#Cc1cc(-c2c(C)cc(F)c3c2CCC3)c(F)c([C@@H](CC(=O)O)NC(=O)[C@H](CC(C)C)n2cc(CCN(C)C)c(C(F)(F)F)cc2=O)c1F] |
| | 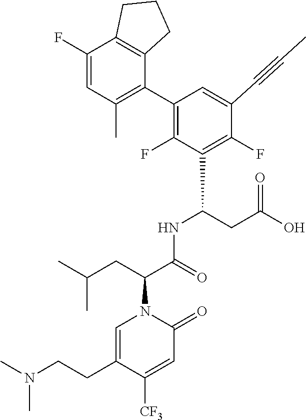 [SMILES: CC#Cc1cc(-c2c(C)cc(F)c3c2CCC3)c(F)c([C@H](CC(=O)O)NC(=O)[C@H](CC(C)C)n2cc(CCN(C)C)c(C(F)(F)F)cc2=O)c1F] |
| | 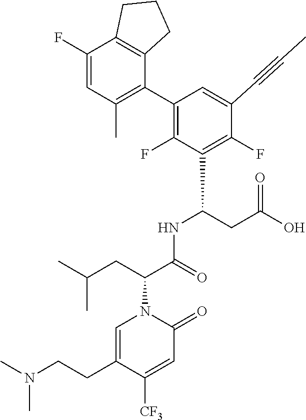 [SMILES: CC#Cc1cc(-c2c(C)cc(F)c3c2CCC3)c(F)c([C@H](CC(=O)O)NC(=O)[C@@H](CC(C)C)n2cc(CCN(C)C)c(C(F)(F)F)cc2=O)c1F] |
| | 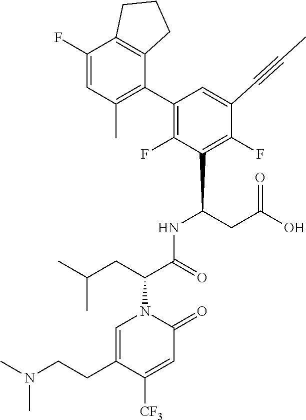 [SMILES: CC#Cc1cc(-c2c(C)cc(F)c3c2CCC3)c(F)c([C@@H](CC(=O)O)NC(=O)[C@@H](CC(C)C)n2cc(CCN(C)C)c(C(F)(F)F)cc2=O)c1F] |
| 199 | 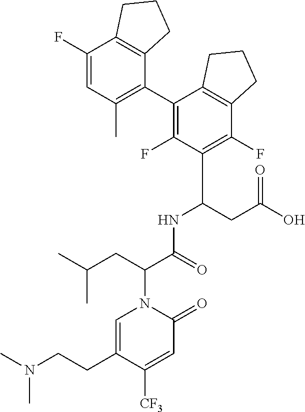 [SMILES: Cc1cc(F)c2c(c1-c1c(F)c(C(CC(=O)O)NC(=O)C(CC(C)C)n3cc(CCN(C)C)c(C(F)(F)F)cc3=O)c(F)c3c1CCC3)CCC2] |
| | 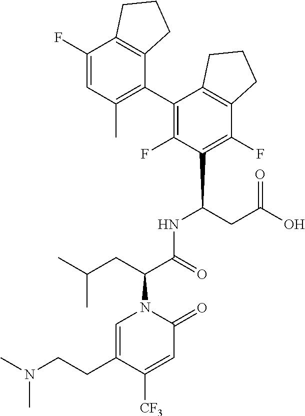 [SMILES: Cc1cc(F)c2c(c1-c1c(F)c([C@@H](CC(=O)O)NC(=O)[C@H](CC(C)C)n3cc(CCN(C)C)c(C(F)(F)F)cc3=O)c(F)c3c1CCC3)CCC2] |
| | 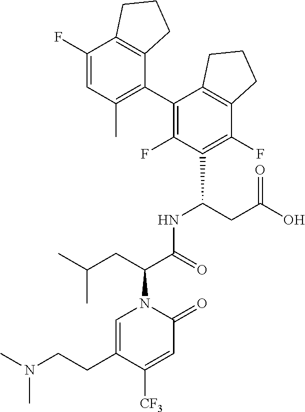 [SMILES: Cc1cc(F)c2c(c1-c1c(F)c([C@H](CC(=O)O)NC(=O)[C@H](CC(C)C)n3cc(CCN(C)C)c(C(F)(F)F)cc3=O)c(F)c3c1CCC3)CCC2] |
| | 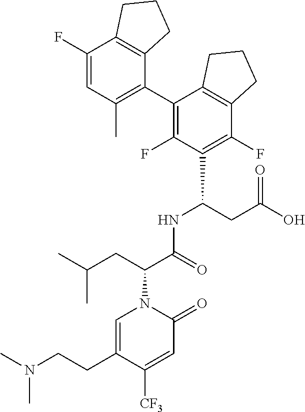 [SMILES: Cc1cc(F)c2c(c1-c1c(F)c([C@H](CC(=O)O)NC(=O)[C@@H](CC(C)C)n3cc(CCN(C)C)c(C(F)(F)F)cc3=O)c(F)c3c1CCC3)CCC2] |
| | 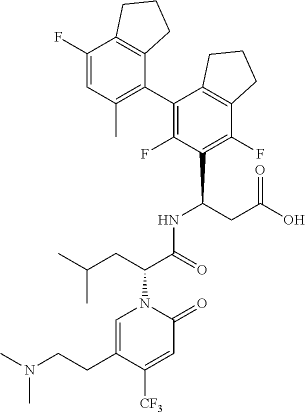 [SMILES: Cc1cc(F)c2c(c1-c1c(F)c([C@@H](CC(=O)O)NC(=O)[C@@H](CC(C)C)n3cc(CCN(C)C)c(C(F)(F)F)cc3=O)c(F)c3c1CCC3)CCC2] |
| 200 | 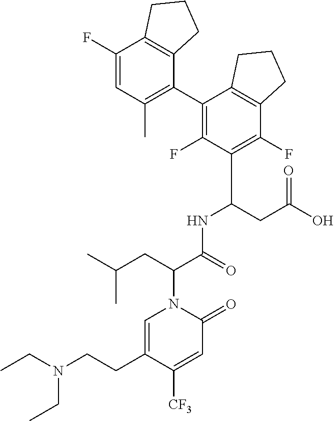 [SMILES: CCN(CC)CCc1cn(C(CC(C)C)C(=O)NC(CC(=O)O)c2c(F)c3c(c(-c4c(C)cc(F)c5c4CCC5)c2F)CCC3)c(=O)cc1C(F)(F)F] |
| | 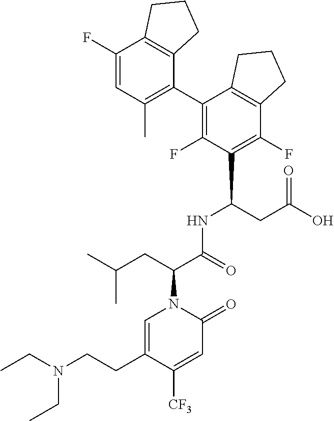 [SMILES: CCN(CC)CCc1cn([C@@H](CC(C)C)C(=O)N[C@H](CC(=O)O)c2c(F)c3c(c(-c4c(C)cc(F)c5c4CCC5)c2F)CCC3)c(=O)cc1C(F)(F)F] |
| | 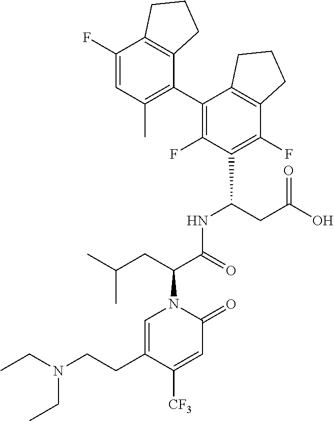 [SMILES: CCN(CC)CCc1cn([C@@H](CC(C)C)C(=O)N[C@@H](CC(=O)O)c2c(F)c3c(c(-c4c(C)cc(F)c5c4CCC5)c2F)CCC3)c(=O)cc1C(F)(F)F] |
| | 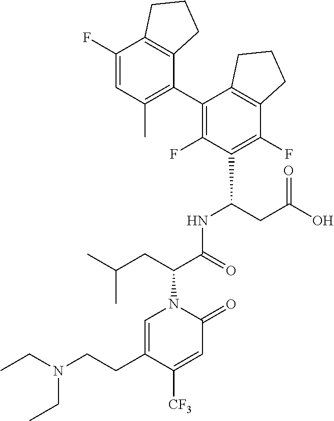 [SMILES: CCN(CC)CCc1cn([C@H](CC(C)C)C(=O)N[C@@H](CC(=O)O)c2c(F)c3c(c(-c4c(C)cc(F)c5c4CCC5)c2F)CCC3)c(=O)cc1C(F)(F)F] |
| | 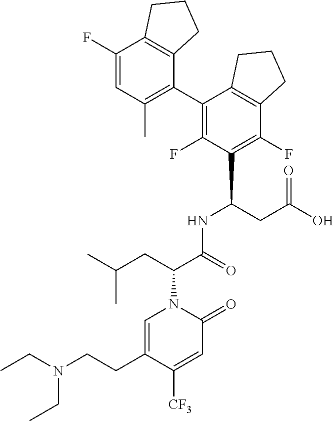 [SMILES: CCN(CC)CCc1cn([C@H](CC(C)C)C(=O)N[C@H](CC(=O)O)c2c(F)c3c(c(-c4c(C)cc(F)c5c4CCC5)c2F)CCC3)c(=O)cc1C(F)(F)F] |
| 201 | 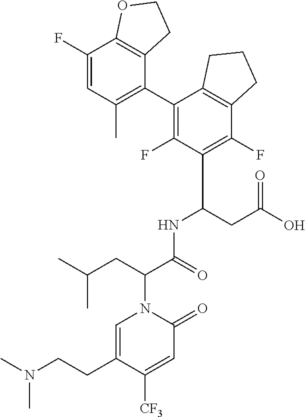 [SMILES: Cc1cc(F)c2c(c1-c1c(F)c(C(CC(=O)O)NC(=O)C(CC(C)C)n3cc(CCN(C)C)c(C(F)(F)F)cc3=O)c(F)c3c1CCC3)CCO2] |
| | 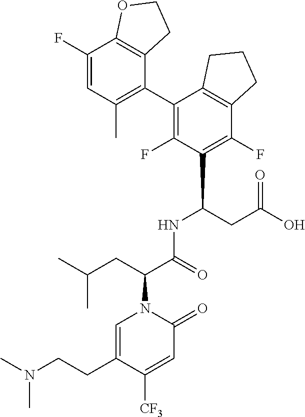 [SMILES: Cc1cc(F)c2c(c1-c1c(F)c([C@@H](CC(=O)O)NC(=O)[C@H](CC(C)C)n3cc(CCN(C)C)c(C(F)(F)F)cc3=O)c(F)c3c1CCC3)CCO2] |
| | 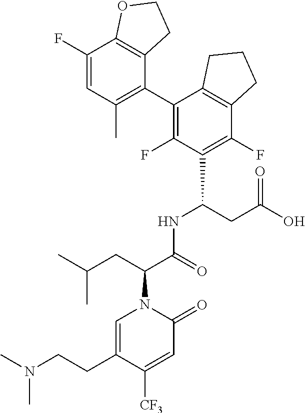 [SMILES: Cc1cc(F)c2c(c1-c1c(F)c([C@H](CC(=O)O)NC(=O)[C@H](CC(C)C)n3cc(CCN(C)C)c(C(F)(F)F)cc3=O)c(F)c3c1CCC3)CCO2] |
| | 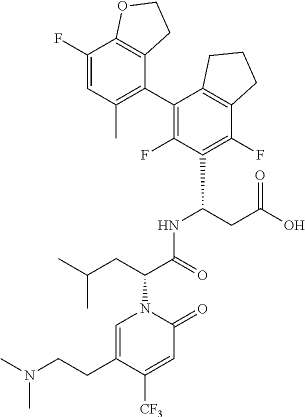 [SMILES: Cc1cc(F)c2c(c1-c1c(F)c([C@H](CC(=O)O)NC(=O)[C@@H](CC(C)C)n3cc(CCN(C)C)c(C(F)(F)F)cc3=O)c(F)c3c1CCC3)CCO2] |
| | 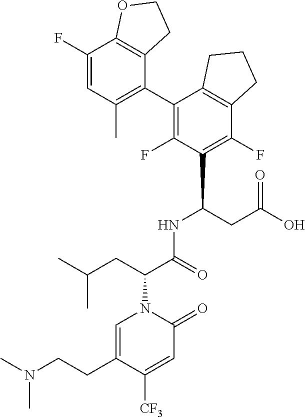 [SMILES: Cc1cc(F)c2c(c1-c1c(F)c([C@@H](CC(=O)O)NC(=O)[C@@H](CC(C)C)n3cc(CCN(C)C)c(C(F)(F)F)cc3=O)c(F)c3c1CCC3)CCO2] |
| 202 | 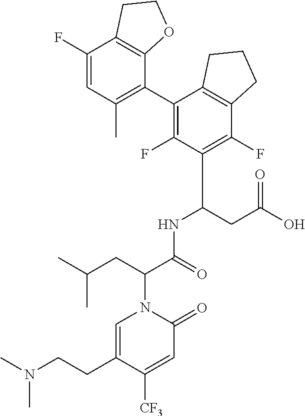 [SMILES: Cc1cc(F)c2c(c1-c1c(F)c(C(CC(=O)O)NC(=O)C(CC(C)C)n3cc(CCN(C)C)c(C(F)(F)F)cc3=O)c(F)c3c1CCC3)OCC2] |
| | 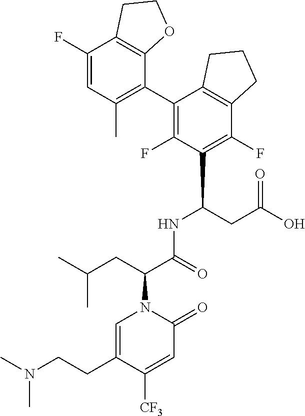 [SMILES: Cc1cc(F)c2c(c1-c1c(F)c([C@@H](CC(=O)O)NC(=O)[C@H](CC(C)C)n3cc(CCN(C)C)c(C(F)(F)F)cc3=O)c(F)c3c1CCC3)OCC2] |
| | 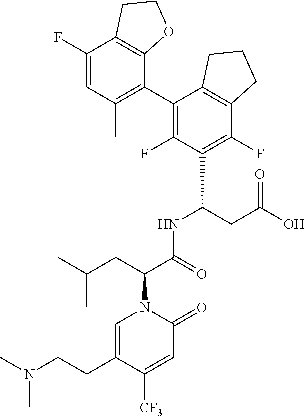 [SMILES: Cc1cc(F)c2c(c1-c1c(F)c([C@H](CC(=O)O)NC(=O)[C@H](CC(C)C)n3cc(CCN(C)C)c(C(F)(F)F)cc3=O)c(F)c3c1CCC3)OCC2] |
| | 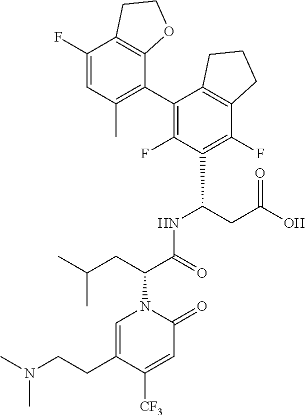 [SMILES: Cc1cc(F)c2c(c1-c1c(F)c([C@H](CC(=O)O)NC(=O)[C@@H](CC(C)C)n3cc(CCN(C)C)c(C(F)(F)F)cc3=O)c(F)c3c1CCC3)OCC2] |
| | 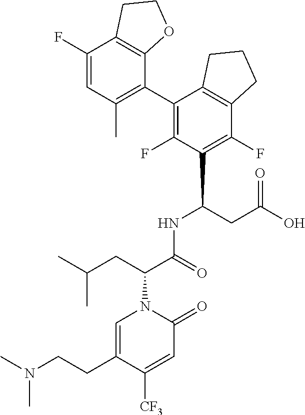 [SMILES: Cc1cc(F)c2c(c1-c1c(F)c([C@@H](CC(=O)O)NC(=O)[C@@H](CC(C)C)n3cc(CCN(C)C)c(C(F)(F)F)cc3=O)c(F)c3c1CCC3)OCC2] |
| 203 | 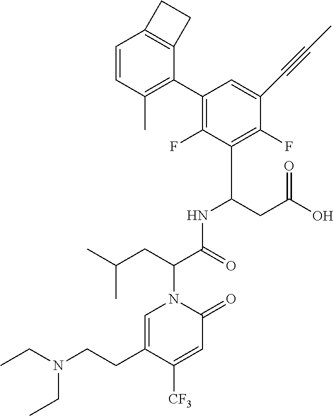 [SMILES: CC#Cc1cc(-c2c(C)ccc3c2CC3)c(F)c(C(CC(=O)O)NC(=O)C(CC(C)C)n2cc(CCN(CC)CC)c(C(F)(F)F)cc2=O)c1F] |
| | 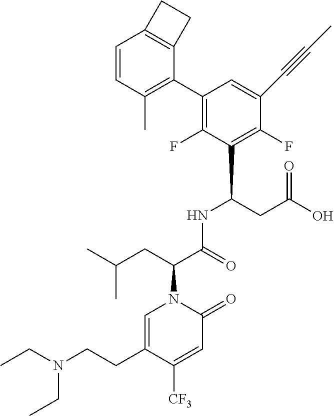 [SMILES: CC#Cc1cc(-c2c(C)ccc3c2CC3)c(F)c([C@@H](CC(=O)O)NC(=O)[C@H](CC(C)C)n2cc(CCN(CC)CC)c(C(F)(F)F)cc2=O)c1F] |
| | 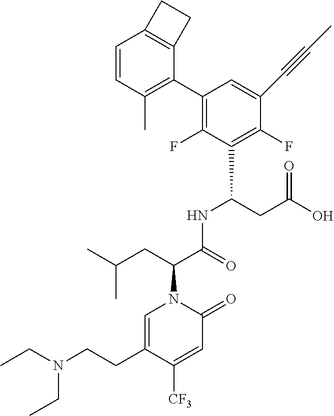 [SMILES: CC#Cc1cc(-c2c(C)ccc3c2CC3)c(F)c([C@H](CC(=O)O)NC(=O)[C@H](CC(C)C)n2cc(CCN(CC)CC)c(C(F)(F)F)cc2=O)c1F] |
| | 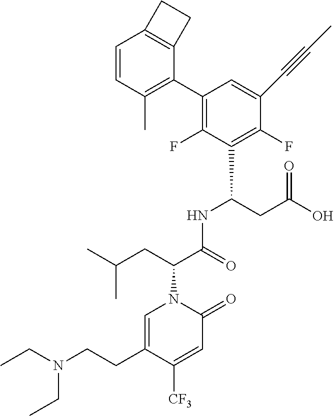 [SMILES: CC#Cc1cc(-c2c(C)ccc3c2CC3)c(F)c([C@H](CC(=O)O)NC(=O)[C@@H](CC(C)C)n2cc(CCN(CC)CC)c(C(F)(F)F)cc2=O)c1F] |
| | 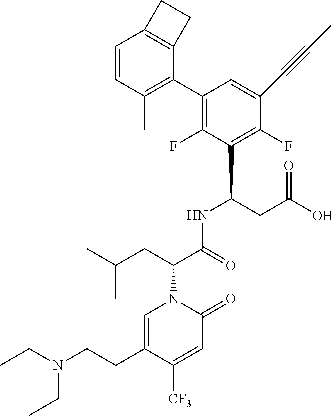 [SMILES: CC#Cc1cc(-c2c(C)ccc3c2CC3)c(F)c([C@@H](CC(=O)O)NC(=O)[C@@H](CC(C)C)n2cc(CCN(CC)CC)c(C(F)(F)F)cc2=O)c1F] |
| 204 | 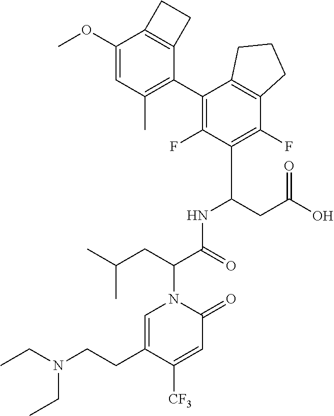 [SMILES: CCN(CC)CCc1cn(C(CC(C)C)C(=O)NC(CC(=O)O)c2c(F)c3c(c(-c4c(C)cc(OC)c5c4CC5)c2F)CCC3)c(=O)cc1C(F)(F)F] |
| | 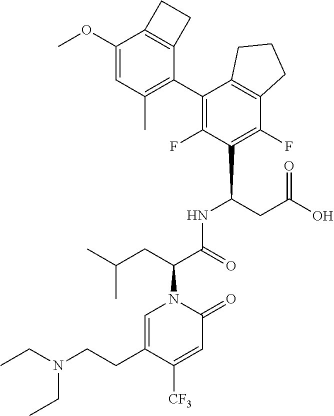 [SMILES: CCN(CC)CCc1cn([C@@H](CC(C)C)C(=O)N[C@H](CC(=O)O)c2c(F)c3c(c(-c4c(C)cc(OC)c5c4CC5)c2F)CCC3)c(=O)cc1C(F)(F)F] |
| | 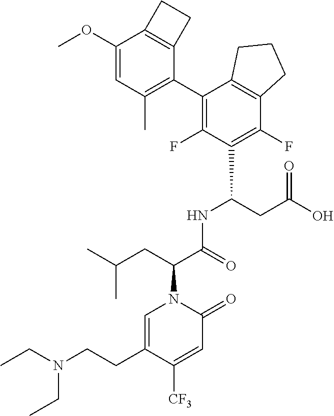 [SMILES: CCN(CC)CCc1cn([C@@H](CC(C)C)C(=O)N[C@@H](CC(=O)O)c2c(F)c3c(c(-c4c(C)cc(OC)c5c4CC5)c2F)CCC3)c(=O)cc1C(F)(F)F] |
| | 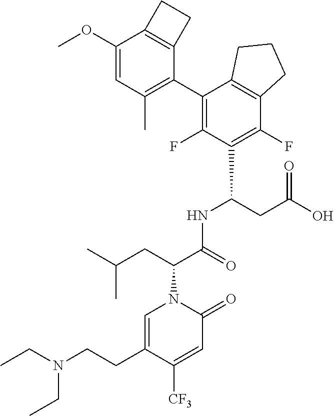 [SMILES: CCN(CC)CCc1cn([C@H](CC(C)C)C(=O)N[C@@H](CC(=O)O)c2c(F)c3c(c(-c4c(C)cc(OC)c5c4CC5)c2F)CCC3)c(=O)cc1C(F)(F)F] |
| | 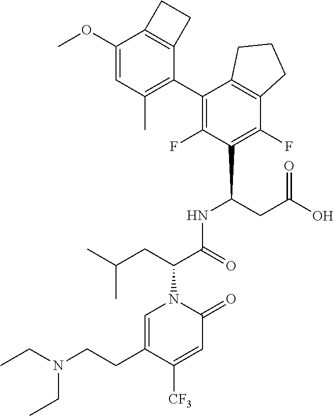 [SMILES: CCN(CC)CCc1cn([C@H](CC(C)C)C(=O)N[C@H](CC(=O)O)c2c(F)c3c(c(-c4c(C)cc(OC)c5c4CC5)c2F)CCC3)c(=O)cc1C(F)(F)F] |
| 205 | 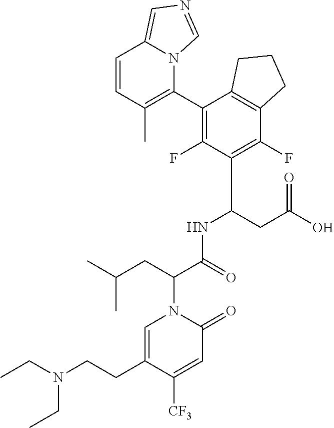 [SMILES: CCN(CC)CCc1cn(C(CC(C)C)C(=O)NC(CC(=O)O)c2c(F)c3c(c(-c4c(C)ccc5cncn45)c2F)CCC3)c(=O)cc1C(F)(F)F] |
| | 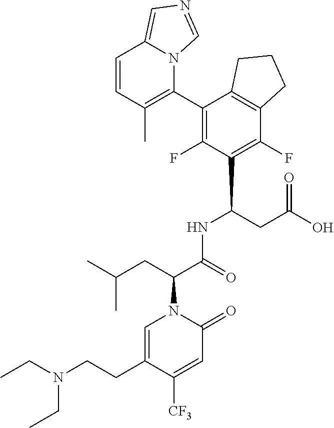 [SMILES: CCN(CC)CCc1cn([C@@H](CC(C)C)C(=O)N[C@H](CC(=O)O)c2c(F)c3c(c(-c4c(C)ccc5cncn45)c2F)CCC3)c(=O)cc1C(F)(F)F] |
| | 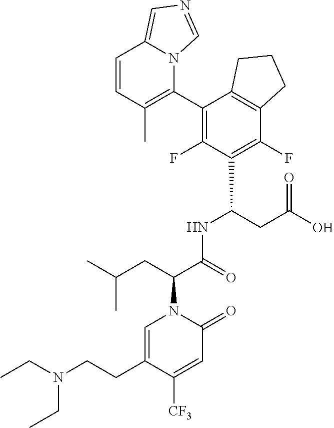 [SMILES: CCN(CC)CCc1cn([C@@H](CC(C)C)C(=O)N[C@@H](CC(=O)O)c2c(F)c3c(c(-c4c(C)ccc5cncn45)c2F)CCC3)c(=O)cc1C(F)(F)F] |
| | 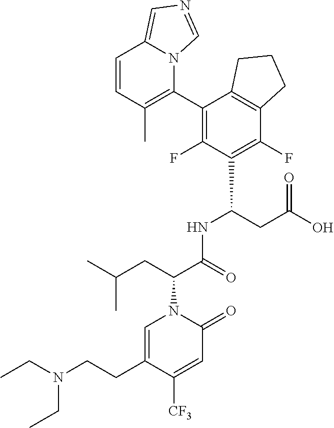 [SMILES: CCN(CC)CCc1cn([C@H](CC(C)C)C(=O)N[C@@H](CC(=O)O)c2c(F)c3c(c(-c4c(C)ccc5cncn45)c2F)CCC3)c(=O)cc1C(F)(F)F] |
| | 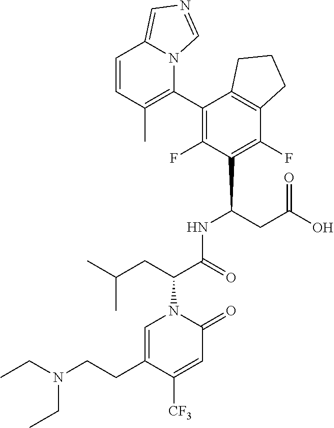 [SMILES: CCN(CC)CCc1cn([C@H](CC(C)C)C(=O)N[C@H](CC(=O)O)c2c(F)c3c(c(-c4c(C)ccc5cncn45)c2F)CCC3)c(=O)cc1C(F)(F)F] |
| 206 | 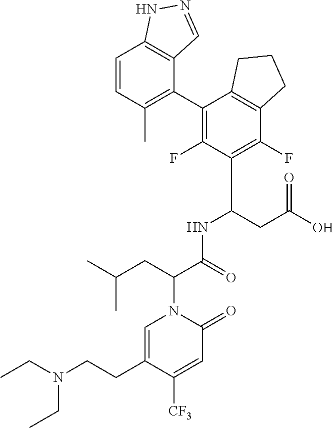 [SMILES: CCN(CC)CCc1cn(C(CC(C)C)C(=O)NC(CC(=O)O)c2c(F)c3c(c(-c4c(C)ccc5[nH]ncc45)c2F)CCC3)c(=O)cc1C(F)(F)F] |
| | 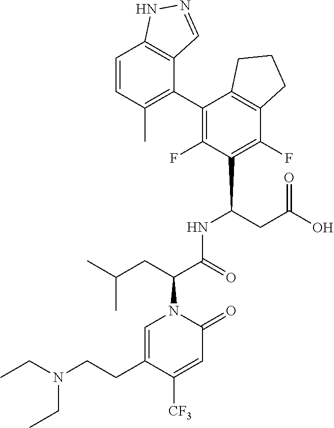 [SMILES: CCN(CC)CCc1cn([C@@H](CC(C)C)C(=O)N[C@H](CC(=O)O)c2c(F)c3c(c(-c4c(C)ccc5[nH]ncc45)c2F)CCC3)c(=O)cc1C(F)(F)F] |
| | 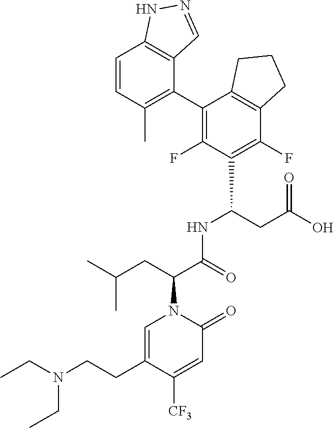 [SMILES: CCN(CC)CCc1cn([C@@H](CC(C)C)C(=O)N[C@@H](CC(=O)O)c2c(F)c3c(c(-c4c(C)ccc5[nH]ncc45)c2F)CCC3)c(=O)cc1C(F)(F)F] |
| | 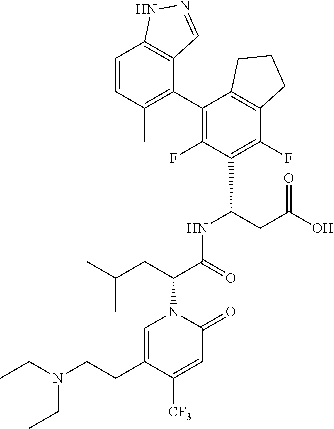 [SMILES: CCN(CC)CCc1cn([C@H](CC(C)C)C(=O)N[C@@H](CC(=O)O)c2c(F)c3c(c(-c4c(C)ccc5[nH]ncc45)c2F)CCC3)c(=O)cc1C(F)(F)F] |
| | 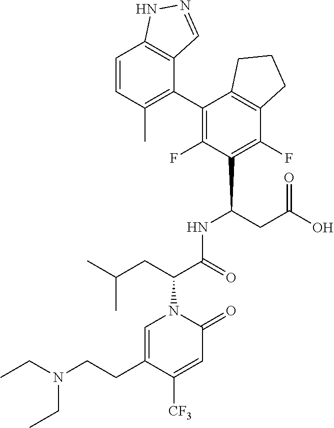 [SMILES: CCN(CC)CCc1cn([C@H](CC(C)C)C(=O)N[C@H](CC(=O)O)c2c(F)c3c(c(-c4c(C)ccc5[nH]ncc45)c2F)CCC3)c(=O)cc1C(F)(F)F] |
| 207 | 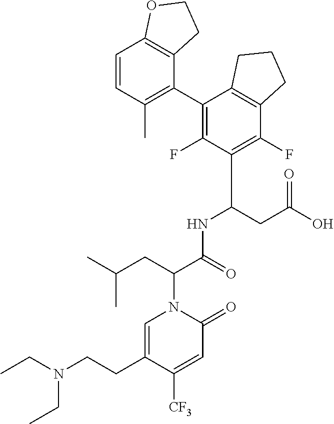 [SMILES: CCN(CC)CCc1cn(C(CC(C)C)C(=O)NC(CC(=O)O)c2c(F)c3c(c(-c4c(C)ccc5c4CCO5)c2F)CCC3)c(=O)cc1C(F)(F)F] |
| | 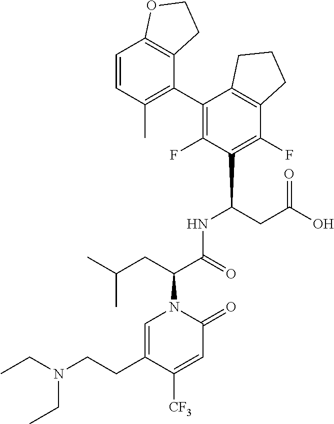 [SMILES: CCN(CC)CCc1cn([C@@H](CC(C)C)C(=O)N[C@H](CC(=O)O)c2c(F)c3c(c(-c4c(C)ccc5c4CCO5)c2F)CCC3)c(=O)cc1C(F)(F)F] |
| | 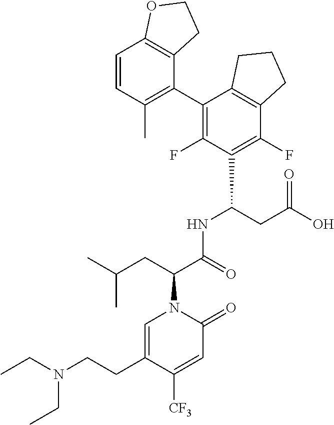 [SMILES: CCN(CC)CCc1cn([C@@H](CC(C)C)C(=O)N[C@@H](CC(=O)O)c2c(F)c3c(c(-c4c(C)ccc5c4CCO5)c2F)CCC3)c(=O)cc1C(F)(F)F] |
| | 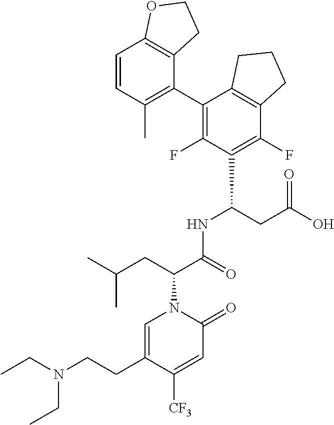 [SMILES: CCN(CC)CCc1cn([C@H](CC(C)C)C(=O)N[C@@H](CC(=O)O)c2c(F)c3c(c(-c4c(C)ccc5c4CCO5)c2F)CCC3)c(=O)cc1C(F)(F)F] |
| | 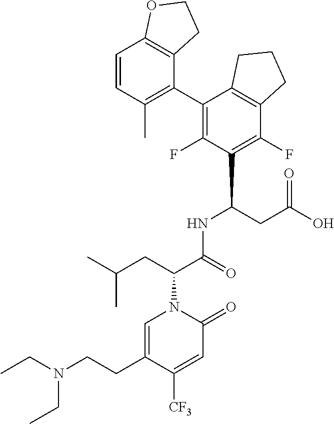 [SMILES: CCN(CC)CCc1cn([C@H](CC(C)C)C(=O)N[C@H](CC(=O)O)c2c(F)c3c(c(-c4c(C)ccc5c4CCO5)c2F)CCC3)c(=O)cc1C(F)(F)F] |
| 208 | 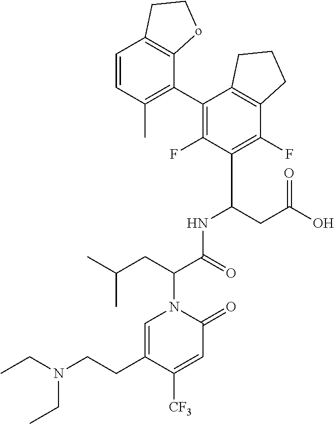 |
| | 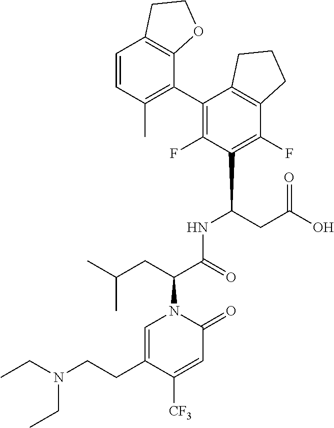 [SMILES: CCN(CC)CCc1cn([C@@H](CC(C)C)C(=O)N[C@H](CC(=O)O)c2c(F)c3c(c(-c4c(C)ccc5c4OCC5)c2F)CCC3)c(=O)cc1C(F)(F)F] |
| | 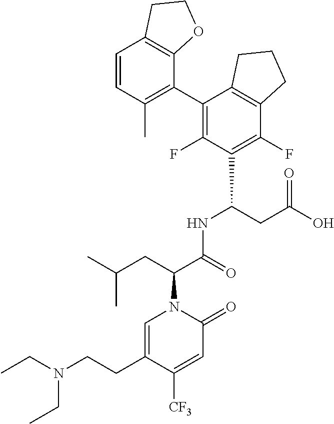 [SMILES: CCN(CC)CCc1cn([C@@H](CC(C)C)C(=O)N[C@@H](CC(=O)O)c2c(F)c3c(c(-c4c(C)ccc5c4OCC5)c2F)CCC3)c(=O)cc1C(F)(F)F] |
| | 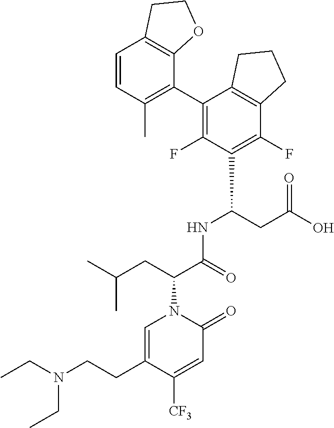 [SMILES: CCN(CC)CCc1cn([C@H](CC(C)C)C(=O)N[C@@H](CC(=O)O)c2c(F)c3c(c(-c4c(C)ccc5c4OCC5)c2F)CCC3)c(=O)cc1C(F)(F)F] |
| | 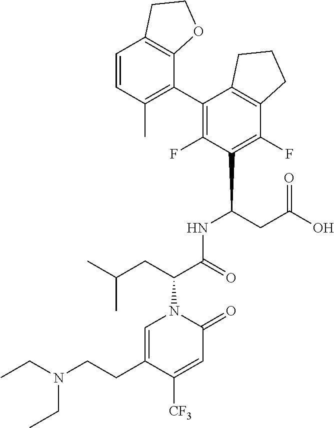 [SMILES: CCN(CC)CCc1cn([C@H](CC(C)C)C(=O)N[C@H](CC(=O)O)c2c(F)c3c(c(-c4c(C)ccc5c4OCC5)c2F)CCC3)c(=O)cc1C(F)(F)F] |
| 209 | 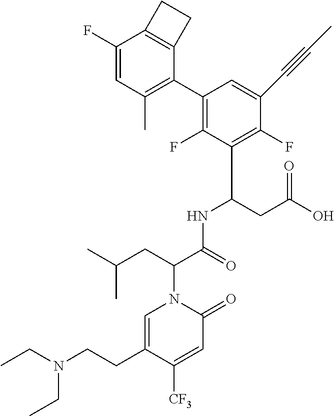 [SMILES: CC#Cc1cc(-c2c(C)cc(F)c3c2CC3)c(F)c(C(CC(=O)O)NC(=O)C(CC(C)C)n2cc(CCN(CC)CC)c(C(F)(F)F)cc2=O)c1F] |
| | 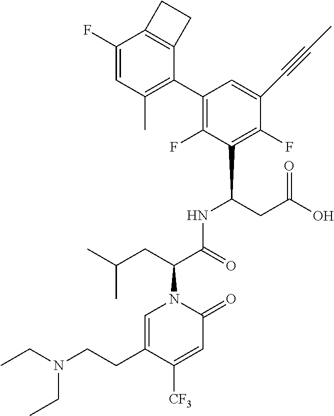 [SMILES: CC#Cc1cc(-c2c(C)cc(F)c3c2CC3)c(F)c([C@@H](CC(=O)O)NC(=O)[C@H](CC(C)C)n2cc(CCN(CC)CC)c(C(F)(F)F)cc2=O)c1F] |
| | 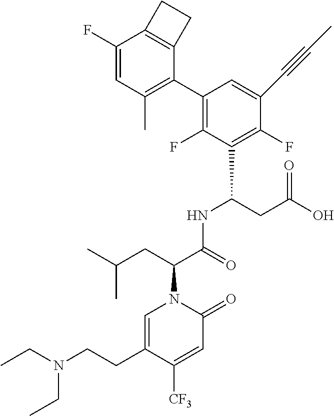 [SMILES: CC#Cc1cc(-c2c(C)cc(F)c3c2CC3)c(F)c([C@H](CC(=O)O)NC(=O)[C@H](CC(C)C)n2cc(CCN(CC)CC)c(C(F)(F)F)cc2=O)c1F] |
| | 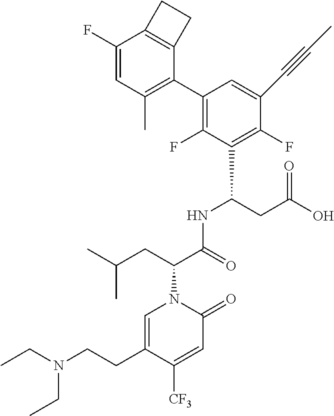 [SMILES: CC#Cc1cc(-c2c(C)cc(F)c3c2CC3)c(F)c([C@H](CC(=O)O)NC(=O)[C@@H](CC(C)C)n2cc(CCN(CC)CC)c(C(F)(F)F)cc2=O)c1F] |
| | 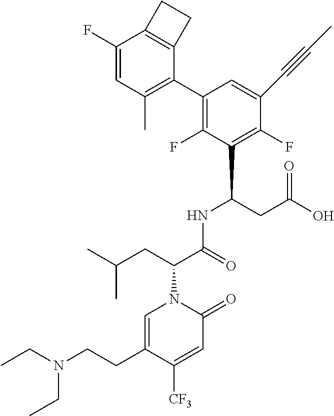 [SMILES: CC#Cc1cc(-c2c(C)cc(F)c3c2CC3)c(F)c([C@@H](CC(=O)O)NC(=O)[C@@H](CC(C)C)n2cc(CCN(CC)CC)c(C(F)(F)F)cc2=O)c1F] |
| 210 | 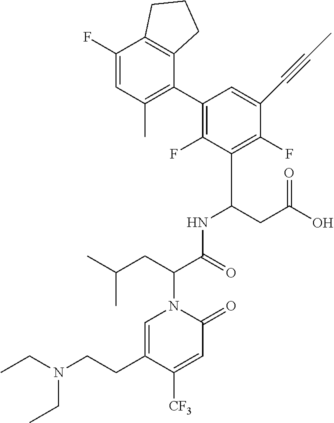 [SMILES: CC#Cc1cc(-c2c(C)cc(F)c3c2CCC3)c(F)c(C(CC(=O)O)NC(=O)C(CC(C)C)n2cc(CCN(CC)CC)c(C(F)(F)F)cc2=O)c1F] |
| | 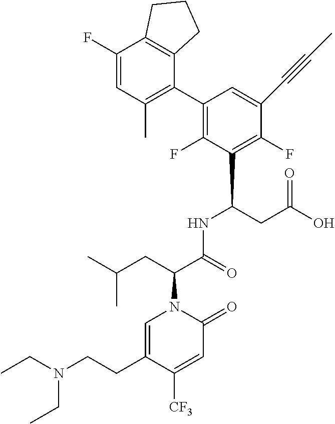 [SMILES: CC#Cc1cc(-c2c(C)cc(F)c3c2CCC3)c(F)c([C@@H](CC(=O)O)NC(=O)[C@H](CC(C)C)n2cc(CCN(CC)CC)c(C(F)(F)F)cc2=O)c1F] |
| | 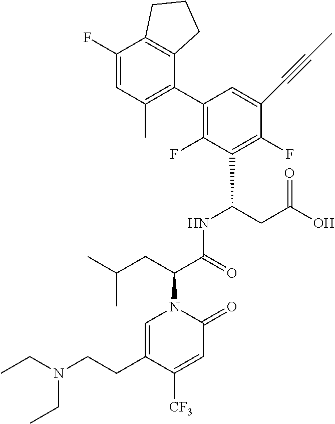 [SMILES: CC#Cc1cc(-c2c(C)cc(F)c3c2CCC3)c(F)c([C@H](CC(=O)O)NC(=O)[C@H](CC(C)C)n2cc(CCN(CC)CC)c(C(F)(F)F)cc2=O)c1F] |
| | 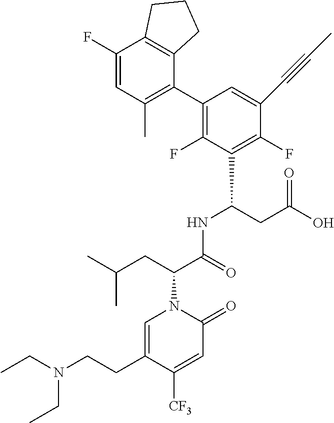 [SMILES: CC#Cc1cc(-c2c(C)cc(F)c3c2CCC3)c(F)c([C@H](CC(=O)O)NC(=O)[C@@H](CC(C)C)n2cc(CCN(CC)CC)c(C(F)(F)F)cc2=O)c1F] |
| | 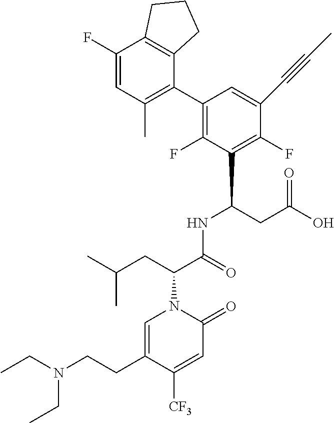 [SMILES: CC#Cc1cc(-c2c(C)cc(F)c3c2CCC3)c(F)c([C@@H](CC(=O)O)NC(=O)[C@@H](CC(C)C)n2cc(CCN(CC)CC)c(C(F)(F)F)cc2=O)c1F] |
| 211 | 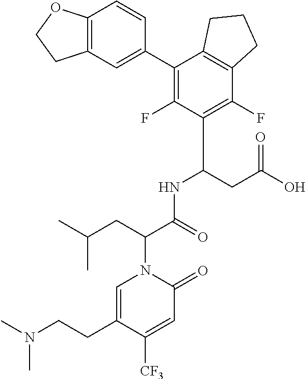 [SMILES: CC(C)CC(C(=O)NC(CC(=O)O)c1c(F)c2c(c(-c3ccc4c(c3)CCO4)c1F)CCC2)n1cc(CCN(C)C)c(C(F)(F)F)cc1=O] |
| | 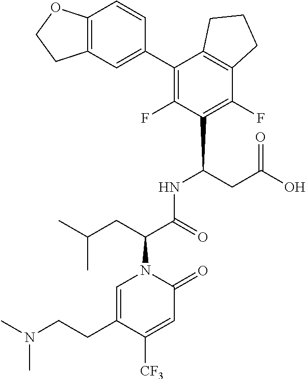 [SMILES: CC(C)C[C@@H](C(=O)N[C@H](CC(=O)O)c1c(F)c2c(c(-c3ccc4c(c3)CCO4)c1F)CCC2)n1cc(CCN(C)C)c(C(F)(F)F)cc1=O] |
| | 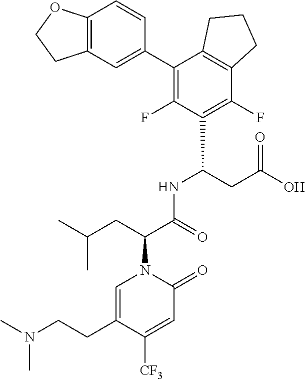 [SMILES: CC(C)C[C@@H](C(=O)N[C@@H](CC(=O)O)c1c(F)c2c(c(-c3ccc4c(c3)CCO4)c1F)CCC2)n1cc(CCN(C)C)c(C(F)(F)F)cc1=O] |
| | 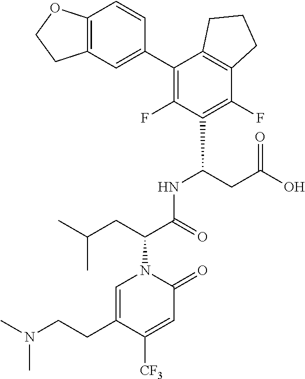 [SMILES: CC(C)C[C@H](C(=O)N[C@@H](CC(=O)O)c1c(F)c2c(c(-c3ccc4c(c3)CCO4)c1F)CCC2)n1cc(CCN(C)C)c(C(F)(F)F)cc1=O] |
| | 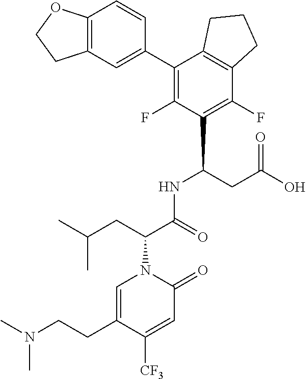 [SMILES: CC(C)C[C@H](C(=O)N[C@H](CC(=O)O)c1c(F)c2c(c(-c3ccc4c(c3)CCO4)c1F)CCC2)n1cc(CCN(C)C)c(C(F)(F)F)cc1=O] |
| 212 | 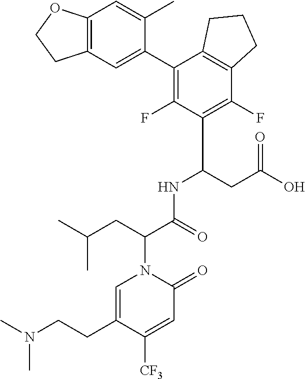 [SMILES: Cc1cc2c(cc1-c1c(F)c(C(CC(=O)O)NC(=O)C(CC(C)C)n3cc(CCN(C)C)c(C(F)(F)F)cc3=O)c(F)c3c1CCC3)CCO2] |
| | 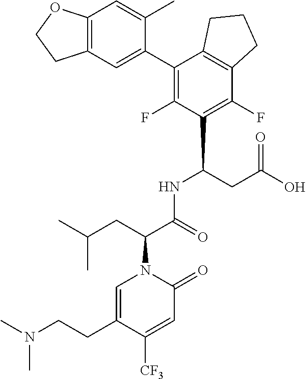 [SMILES: Cc1cc2c(cc1-c1c(F)c([C@@H](CC(=O)O)NC(=O)[C@H](CC(C)C)n3cc(CCN(C)C)c(C(F)(F)F)cc3=O)c(F)c3c1CCC3)CCO2] |
| | 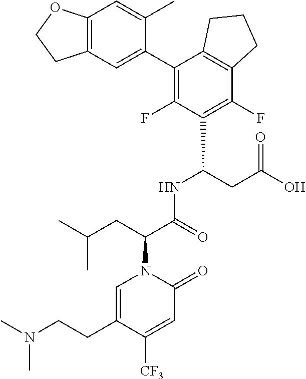 [SMILES: Cc1cc2c(cc1-c1c(F)c([C@H](CC(=O)O)NC(=O)[C@H](CC(C)C)n3cc(CCN(C)C)c(C(F)(F)F)cc3=O)c(F)c3c1CCC3)CCO2] |
| | 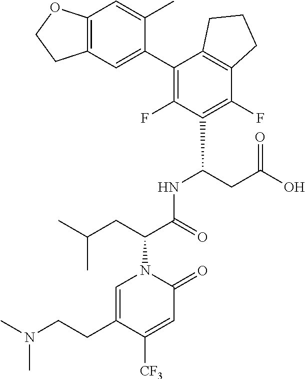 [SMILES: Cc1cc2c(cc1-c1c(F)c([C@H](CC(=O)O)NC(=O)[C@@H](CC(C)C)n3cc(CCN(C)C)c(C(F)(F)F)cc3=O)c(F)c3c1CCC3)CCO2] |
| | 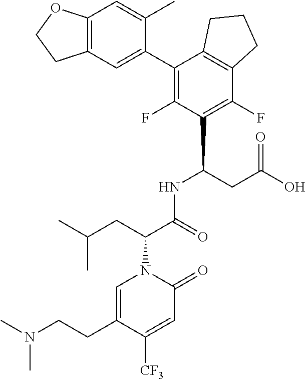 [SMILES: Cc1cc2c(cc1-c1c(F)c([C@@H](CC(=O)O)NC(=O)[C@@H](CC(C)C)n3cc(CCN(C)C)c(C(F)(F)F)cc3=O)c(F)c3c1CCC3)CCO2] |
| 213 | 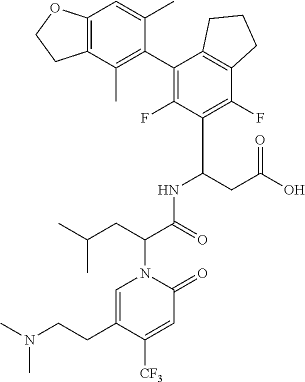 [SMILES: Cc1cc2c(c(C)c1-c1c(F)c(C(CC(=O)O)NC(=O)C(CC(C)C)n3cc(CCN(C)C)c(C(F)(F)F)cc3=O)c(F)c3c1CCC3)CCO2] |
| | 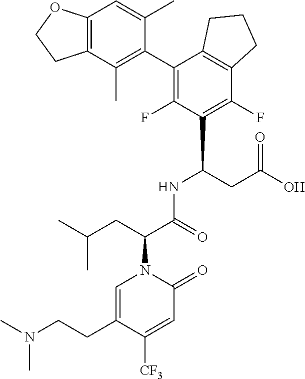 [SMILES: Cc1cc2c(c(C)c1-c1c(F)c([C@@H](CC(=O)O)NC(=O)[C@H](CC(C)C)n3cc(CCN(C)C)c(C(F)(F)F)cc3=O)c(F)c3c1CCC3)CCO2] |
| | 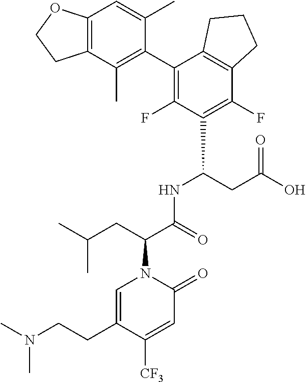 [SMILES: Cc1cc2c(c(C)c1-c1c(F)c([C@H](CC(=O)O)NC(=O)[C@H](CC(C)C)n3cc(CCN(C)C)c(C(F)(F)F)cc3=O)c(F)c3c1CCC3)CCO2] |
| | 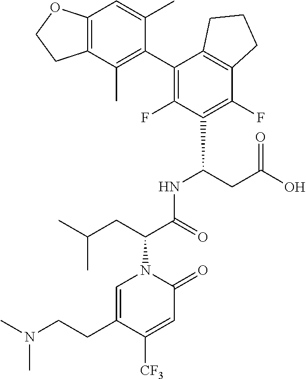 [SMILES: Cc1cc2c(c(C)c1-c1c(F)c([C@H](CC(=O)O)NC(=O)[C@@H](CC(C)C)n3cc(CCN(C)C)c(C(F)(F)F)cc3=O)c(F)c3c1CCC3)CCO2] |
| | 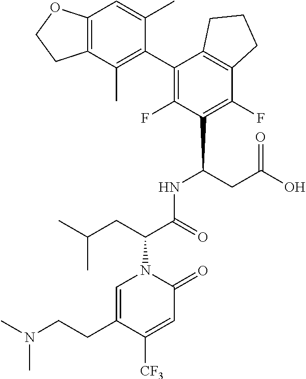 [SMILES: Cc1cc2c(c(C)c1-c1c(F)c([C@@H](CC(=O)O)NC(=O)[C@@H](CC(C)C)n3cc(CCN(C)C)c(C(F)(F)F)cc3=O)c(F)c3c1CCC3)CCO2] |
| 214 | 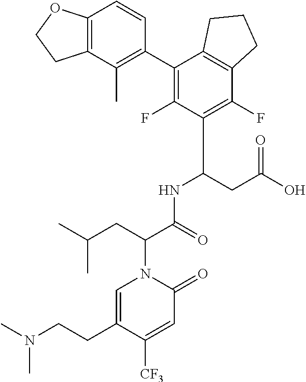 [SMILES: Cc1c(-c2c(F)c(C(CC(=O)O)NC(=O)C(CC(C)C)n3cc(CCN(C)C)c(C(F)(F)F)cc3=O)c(F)c3c2CCC3)ccc2c1CCO2] |
| | 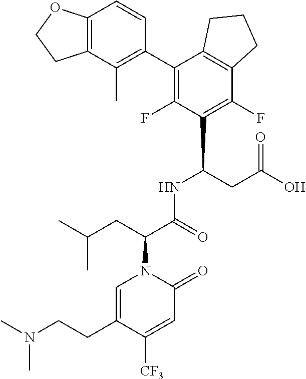 [SMILES: Cc1c(-c2c(F)c([C@@H](CC(=O)O)NC(=O)[C@H](CC(C)C)n3cc(CCN(C)C)c(C(F)(F)F)cc3=O)c(F)c3c2CCC3)ccc2c1CCO2] |
| | 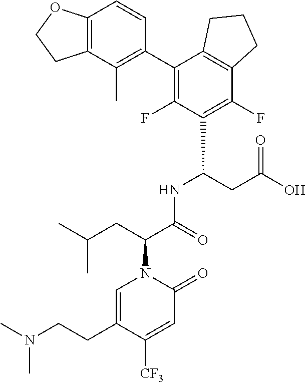 [SMILES: Cc1c(-c2c(F)c([C@H](CC(=O)O)NC(=O)[C@H](CC(C)C)n3cc(CCN(C)C)c(C(F)(F)F)cc3=O)c(F)c3c2CCC3)ccc2c1CCO2] |
| | 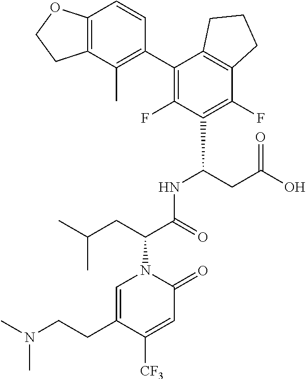 [SMILES: Cc1c(-c2c(F)c([C@H](CC(=O)O)NC(=O)[C@@H](CC(C)C)n3cc(CCN(C)C)c(C(F)(F)F)cc3=O)c(F)c3c2CCC3)ccc2c1CCO2] |
| | 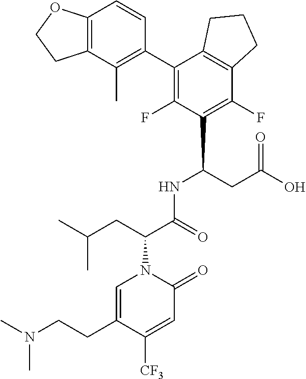 [SMILES: Cc1c(-c2c(F)c([C@@H](CC(=O)O)NC(=O)[C@@H](CC(C)C)n3cc(CCN(C)C)c(C(F)(F)F)cc3=O)c(F)c3c2CCC3)ccc2c1CCO2] |
| 215 | 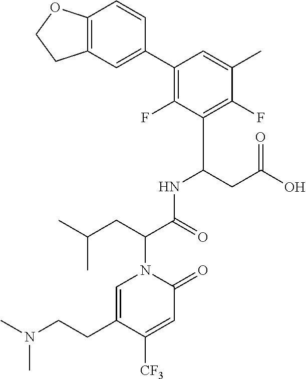 [SMILES: Cc1cc(-c2ccc3c(c2)CCO3)c(F)c(C(CC(=O)O)NC(=O)C(CC(C)C)n2cc(CCN(C)C)c(C(F)(F)F)cc2=O)c1F] |
| | 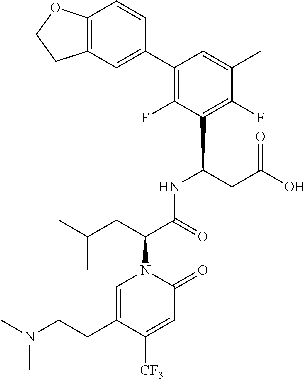 [SMILES: Cc1cc(-c2ccc3c(c2)CCO3)c(F)c([C@@H](CC(=O)O)NC(=O)[C@H](CC(C)C)n2cc(CCN(C)C)c(C(F)(F)F)cc2=O)c1F] |
| | 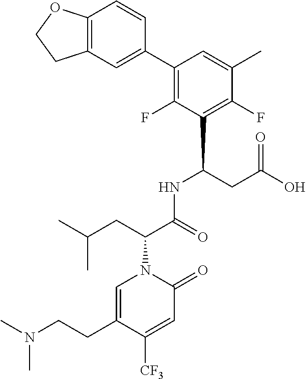 [SMILES: Cc1cc(-c2ccc3c(c2)CCO3)c(F)c([C@@H](CC(=O)O)NC(=O)[C@@H](CC(C)C)n2cc(CCN(C)C)c(C(F)(F)F)cc2=O)c1F] |
| | 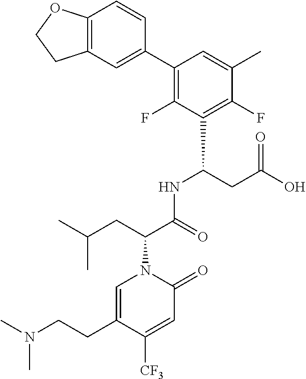 [SMILES: Cc1cc(-c2ccc3c(c2)CCO3)c(F)c([C@H](CC(=O)O)NC(=O)[C@@H](CC(C)C)n2cc(CCN(C)C)c(C(F)(F)F)cc2=O)c1F] |
| | 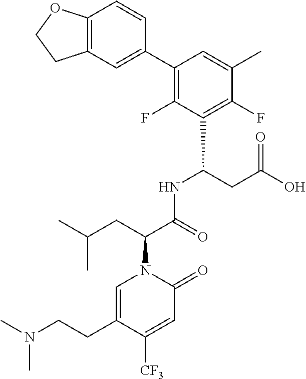 [SMILES: Cc1cc(-c2ccc3c(c2)CCO3)c(F)c([C@H](CC(=O)O)NC(=O)[C@H](CC(C)C)n2cc(CCN(C)C)c(C(F)(F)F)cc2=O)c1F] |
| 216 | 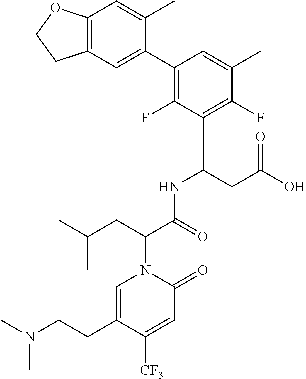 [SMILES: Cc1cc2c(cc1-c1cc(C)c(F)c(C(CC(=O)O)NC(=O)C(CC(C)C)n3cc(CCN(C)C)c(C(F)(F)F)cc3=O)c1F)CCO2] |
| | 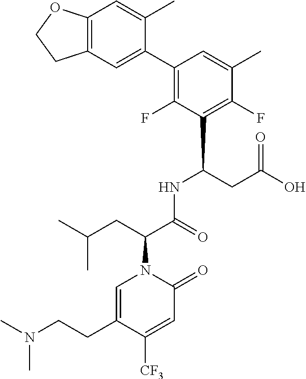 [SMILES: Cc1cc2c(cc1-c1cc(C)c(F)c([C@@H](CC(=O)O)NC(=O)[C@H](CC(C)C)n3cc(CCN(C)C)c(C(F)(F)F)cc3=O)c1F)CCO2] |
| | 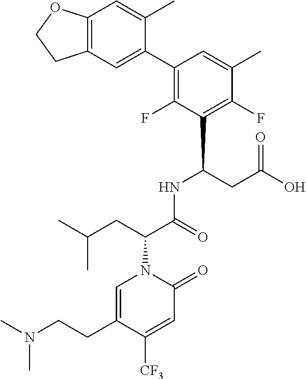 [SMILES: Cc1cc2c(cc1-c1cc(C)c(F)c([C@@H](CC(=O)O)NC(=O)[C@@H](CC(C)C)n3cc(CCN(C)C)c(C(F)(F)F)cc3=O)c1F)CCO2] |
| | 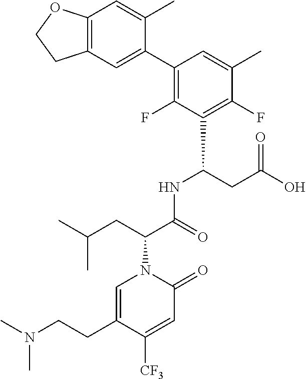 [SMILES: Cc1cc2c(cc1-c1cc(C)c(F)c([C@H](CC(=O)O)NC(=O)[C@@H](CC(C)C)n3cc(CCN(C)C)c(C(F)(F)F)cc3=O)c1F)CCO2] |
| | 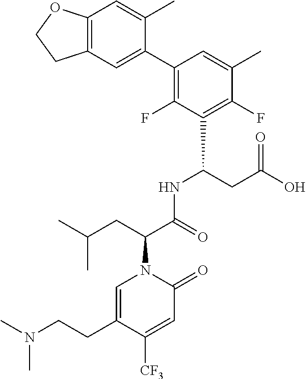 [SMILES: Cc1cc2c(cc1-c1cc(C)c(F)c([C@H](CC(=O)O)NC(=O)[C@H](CC(C)C)n3cc(CCN(C)C)c(C(F)(F)F)cc3=O)c1F)CCO2] |
| 217 | 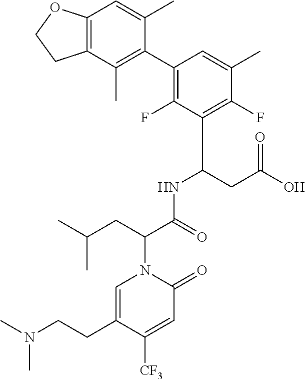 [SMILES: Cc1cc(-c2c(C)cc3c(c2C)CCO3)c(F)c(C(CC(=O)O)NC(=O)C(CC(C)C)n2cc(CCN(C)C)c(C(F)(F)F)cc2=O)c1F] |
| | 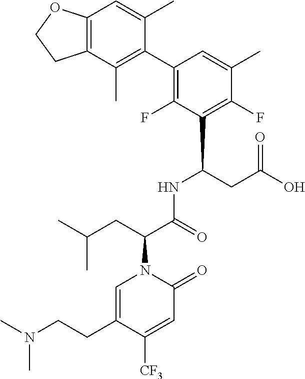 [SMILES: Cc1cc(-c2c(C)cc3c(c2C)CCO3)c(F)c([C@@H](CC(=O)O)NC(=O)[C@H](CC(C)C)n2cc(CCN(C)C)c(C(F)(F)F)cc2=O)c1F] |
| | 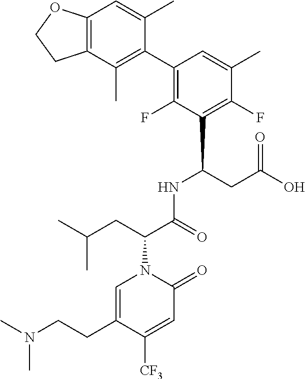 [SMILES: Cc1cc(-c2c(C)cc3c(c2C)CCO3)c(F)c([C@@H](CC(=O)O)NC(=O)[C@@H](CC(C)C)n2cc(CCN(C)C)c(C(F)(F)F)cc2=O)c1F] |
| | 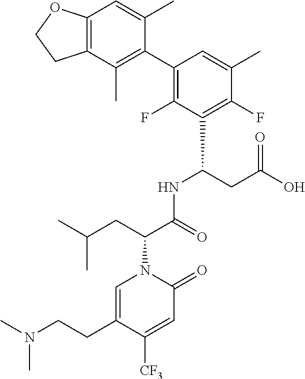 [SMILES: Cc1cc(-c2c(C)cc3c(c2C)CCO3)c(F)c([C@H](CC(=O)O)NC(=O)[C@@H](CC(C)C)n2cc(CCN(C)C)c(C(F)(F)F)cc2=O)c1F] |
| | 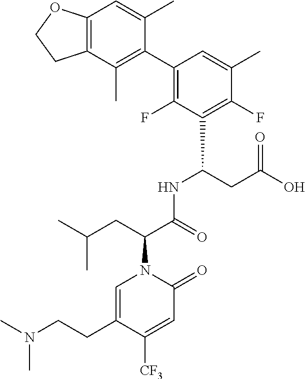 [SMILES: Cc1cc(-c2c(C)cc3c(c2C)CCO3)c(F)c([C@H](CC(=O)O)NC(=O)[C@H](CC(C)C)n2cc(CCN(C)C)c(C(F)(F)F)cc2=O)c1F] |
| 218 | 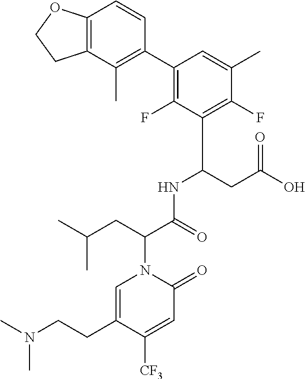 [SMILES: Cc1cc(-c2ccc3c(c2C)CCO3)c(F)c(C(CC(=O)O)NC(=O)C(CC(C)C)n2cc(CCN(C)C)c(C(F)(F)F)cc2=O)c1F] |
| | 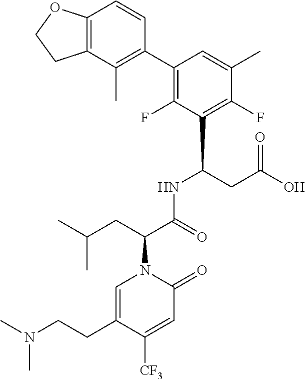 [SMILES: Cc1cc(-c2ccc3c(c2C)CCO3)c(F)c([C@@H](CC(=O)O)NC(=O)[C@H](CC(C)C)n2cc(CCN(C)C)c(C(F)(F)F)cc2=O)c1F] |
| | 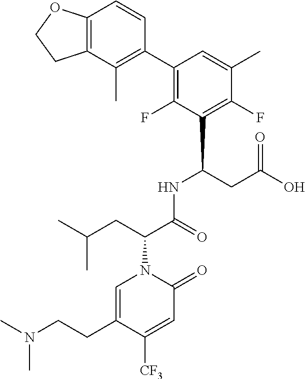 [SMILES: Cc1cc(-c2ccc3c(c2C)CCO3)c(F)c([C@@H](CC(=O)O)NC(=O)[C@@H](CC(C)C)n2cc(CCN(C)C)c(C(F)(F)F)cc2=O)c1F] |
| | 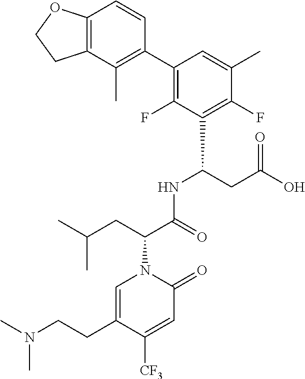 [SMILES: Cc1cc(-c2ccc3c(c2C)CCO3)c(F)c([C@H](CC(=O)O)NC(=O)[C@@H](CC(C)C)n2cc(CCN(C)C)c(C(F)(F)F)cc2=O)c1F] |
| | 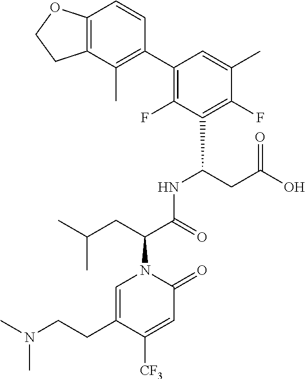 [SMILES: Cc1cc(-c2ccc3c(c2C)CCO3)c(F)c([C@H](CC(=O)O)NC(=O)[C@H](CC(C)C)n2cc(CCN(C)C)c(C(F)(F)F)cc2=O)c1F] |
| 219 | 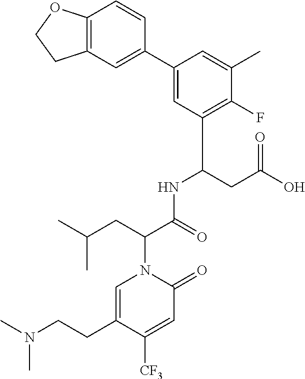 [SMILES: Cc1cc(-c2ccc3c(c2)CCO3)cc(C(CC(=O)O)NC(=O)C(CC(C)C)n2cc(CCN(C)C)c(C(F)(F)F)cc2=O)c1F] |
| | 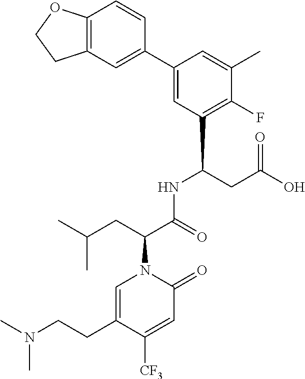 [SMILES: Cc1cc(-c2ccc3c(c2)CCO3)cc([C@@H](CC(=O)O)NC(=O)[C@H](CC(C)C)n2cc(CCN(C)C)c(C(F)(F)F)cc2=O)c1F] |
| | 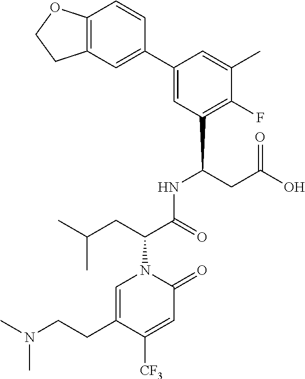 [SMILES: Cc1cc(-c2ccc3c(c2)CCO3)cc([C@@H](CC(=O)O)NC(=O)[C@@H](CC(C)C)n2cc(CCN(C)C)c(C(F)(F)F)cc2=O)c1F] |
| | 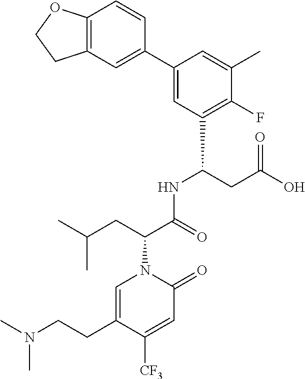 [SMILES: Cc1cc(-c2ccc3c(c2)CCO3)cc([C@H](CC(=O)O)NC(=O)[C@@H](CC(C)C)n2cc(CCN(C)C)c(C(F)(F)F)cc2=O)c1F] |
| | 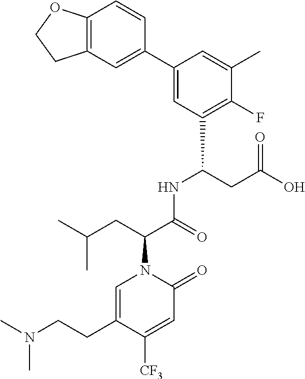 [SMILES: Cc1cc(-c2ccc3c(c2)CCO3)cc([C@H](CC(=O)O)NC(=O)[C@H](CC(C)C)n2cc(CCN(C)C)c(C(F)(F)F)cc2=O)c1F] |
| 220 | 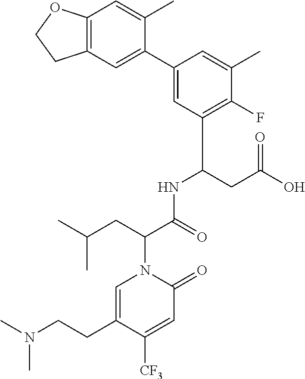 [SMILES: Cc1cc2c(cc1-c1cc(C)c(F)c(C(CC(=O)O)NC(=O)C(CC(C)C)n3cc(CCN(C)C)c(C(F)(F)F)cc3=O)c1)CCO2] |
| | 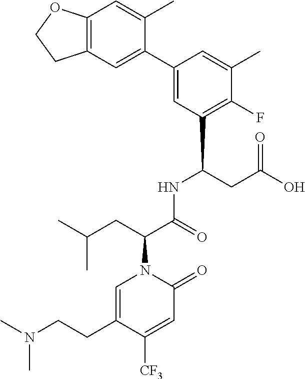 [SMILES: Cc1cc2c(cc1-c1cc(C)c(F)c([C@@H](CC(=O)O)NC(=O)[C@H](CC(C)C)n3cc(CCN(C)C)c(C(F)(F)F)cc3=O)c1)CCO2] |
| | 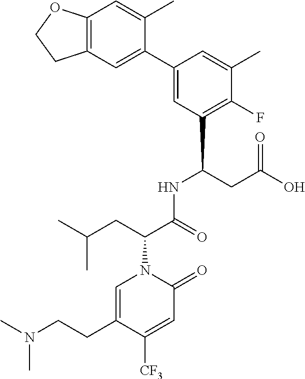 [SMILES: Cc1cc2c(cc1-c1cc(C)c(F)c([C@@H](CC(=O)O)NC(=O)[C@@H](CC(C)C)n3cc(CCN(C)C)c(C(F)(F)F)cc3=O)c1)CCO2] |
| | 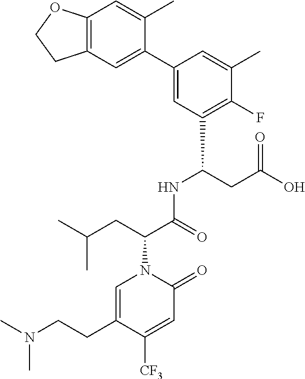 [SMILES: Cc1cc2c(cc1-c1cc(C)c(F)c([C@H](CC(=O)O)NC(=O)[C@@H](CC(C)C)n3cc(CCN(C)C)c(C(F)(F)F)cc3=O)c1)CCO2] |
| | 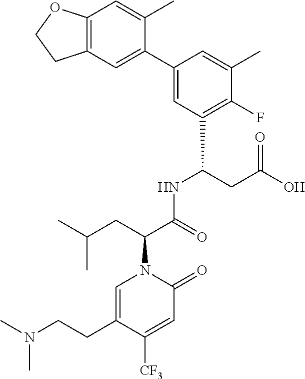 [SMILES: Cc1cc2c(cc1-c1cc(C)c(F)c([C@H](CC(=O)O)NC(=O)[C@H](CC(C)C)n3cc(CCN(C)C)c(C(F)(F)F)cc3=O)c1)CCO2] |
| 221 | 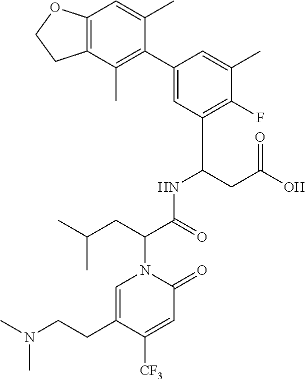 [SMILES: Cc1cc(-c2c(C)cc3c(c2C)CCO3)cc(C(CC(=O)O)NC(=O)C(CC(C)C)n2cc(CCN(C)C)c(C(F)(F)F)cc2=O)c1F] |
| | 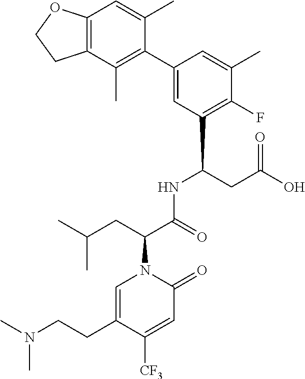 [SMILES: Cc1cc(-c2c(C)cc3c(c2C)CCO3)cc([C@@H](CC(=O)O)NC(=O)[C@H](CC(C)C)n2cc(CCN(C)C)c(C(F)(F)F)cc2=O)c1F] |
| | 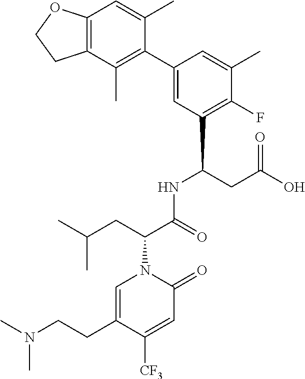 [SMILES: Cc1cc(-c2c(C)cc3c(c2C)CCO3)cc([C@@H](CC(=O)O)NC(=O)[C@@H](CC(C)C)n2cc(CCN(C)C)c(C(F)(F)F)cc2=O)c1F] |
| | 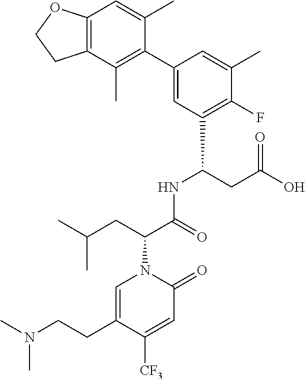 [SMILES: Cc1cc(-c2c(C)cc3c(c2C)CCO3)cc([C@H](CC(=O)O)NC(=O)[C@@H](CC(C)C)n2cc(CCN(C)C)c(C(F)(F)F)cc2=O)c1F] |
| | 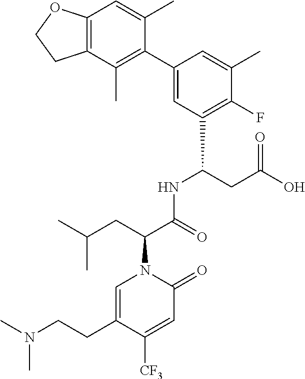 [SMILES: Cc1cc(-c2c(C)cc3c(c2C)CCO3)cc([C@H](CC(=O)O)NC(=O)[C@H](CC(C)C)n2cc(CCN(C)C)c(C(F)(F)F)cc2=O)c1F] |
| 222 | 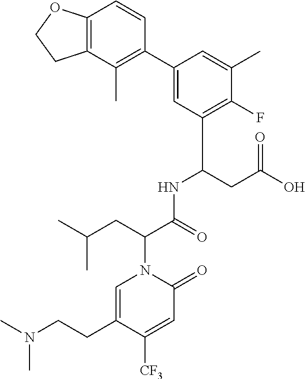 [SMILES: Cc1cc(-c2ccc3c(c2C)CCO3)cc(C(CC(=O)O)NC(=O)C(CC(C)C)n2cc(CCN(C)C)c(C(F)(F)F)cc2=O)c1F] |
| | 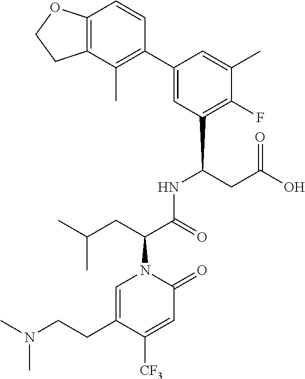 [SMILES: Cc1cc(-c2ccc3c(c2C)CCO3)cc([C@@H](CC(=O)O)NC(=O)[C@H](CC(C)C)n2cc(CCN(C)C)c(C(F)(F)F)cc2=O)c1F] |
| | 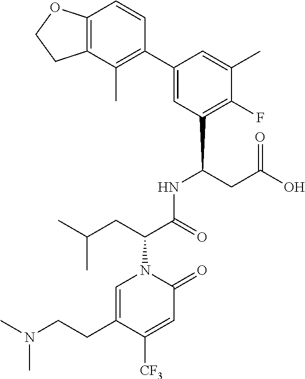 [SMILES: Cc1cc(-c2ccc3c(c2C)CCO3)cc([C@@H](CC(=O)O)NC(=O)[C@@H](CC(C)C)n2cc(CCN(C)C)c(C(F)(F)F)cc2=O)c1F] |
| | 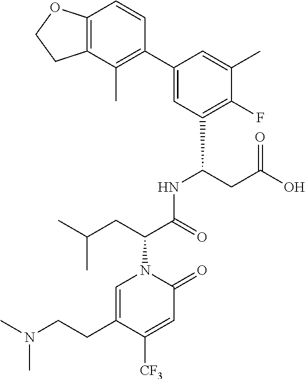 [SMILES: Cc1cc(-c2ccc3c(c2C)CCO3)cc([C@H](CC(=O)O)NC(=O)[C@@H](CC(C)C)n2cc(CCN(C)C)c(C(F)(F)F)cc2=O)c1F] |
| | 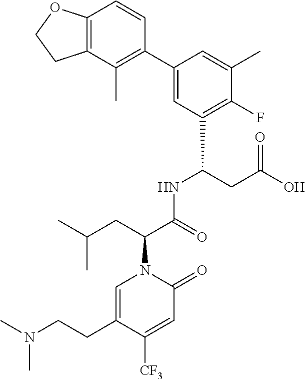 [SMILES: Cc1cc(-c2ccc3c(c2C)CCO3)cc([C@H](CC(=O)O)NC(=O)[C@H](CC(C)C)n2cc(CCN(C)C)c(C(F)(F)F)cc2=O)c1F] |
| 223 | 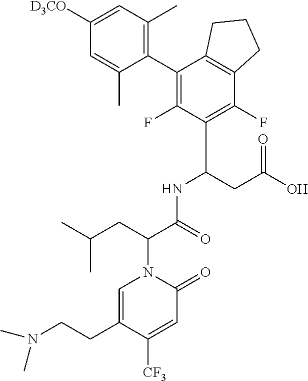 |
| | 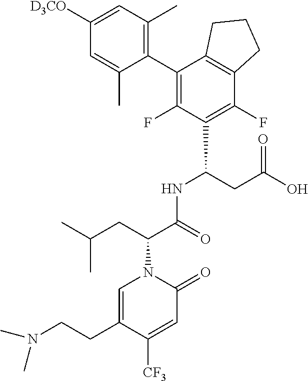 |
| | 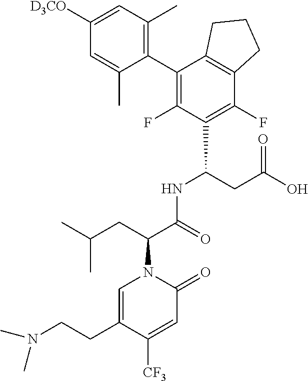 |
| | 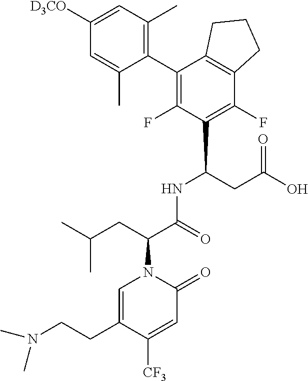 |
| | 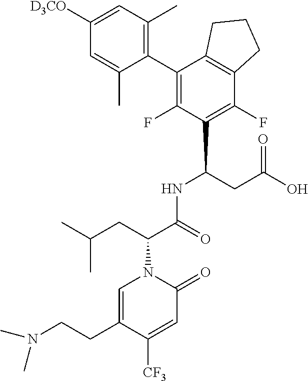 |
| 224 | 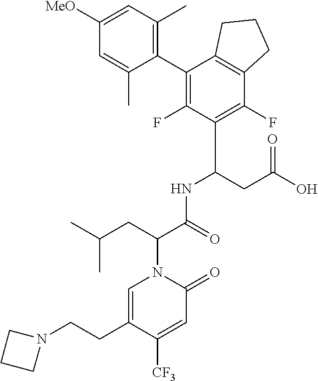 [SMILES: COc1cc(C)c(-c2c(F)c(C(CC(=O)O)NC(=O)C(CC(C)C)n3cc(CCN4CCC4)c(C(F)(F)F)cc3=O)c(F)c3c2CCC3)c(C)c1] |
| | 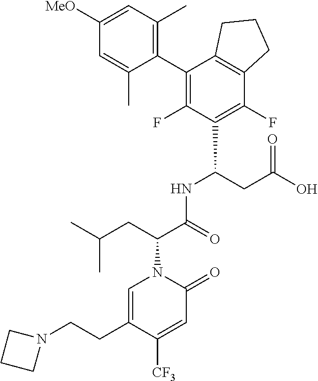 [SMILES: COc1cc(C)c(-c2c(F)c([C@H](CC(=O)O)NC(=O)[C@@H](CC(C)C)n3cc(CCN4CCC4)c(C(F)(F)F)cc3=O)c(F)c3c2CCC3)c(C)c1] |
| | 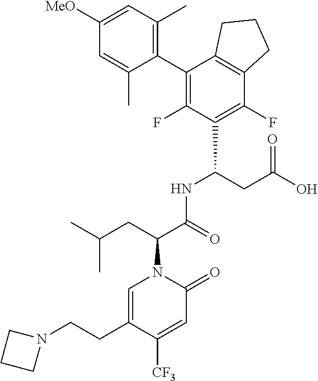 [SMILES: COc1cc(C)c(-c2c(F)c([C@H](CC(=O)O)NC(=O)[C@H](CC(C)C)n3cc(CCN4CCC4)c(C(F)(F)F)cc3=O)c(F)c3c2CCC3)c(C)c1] |
| | 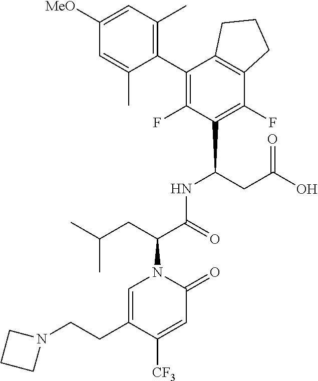 [SMILES: COc1cc(C)c(-c2c(F)c([C@@H](CC(=O)O)NC(=O)[C@H](CC(C)C)n3cc(CCN4CCC4)c(C(F)(F)F)cc3=O)c(F)c3c2CCC3)c(C)c1] |
| | 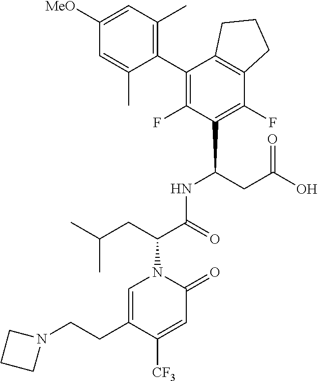 [SMILES: COc1cc(C)c(-c2c(F)c([C@@H](CC(=O)O)NC(=O)[C@@H](CC(C)C)n3cc(CCN4CCC4)c(C(F)(F)F)cc3=O)c(F)c3c2CCC3)c(C)c1] |
| 225 | 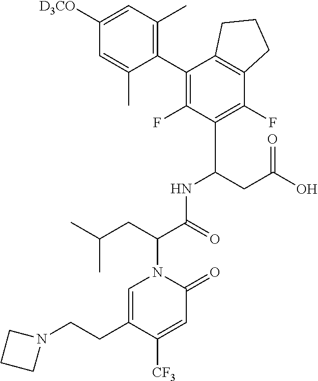 |
| | 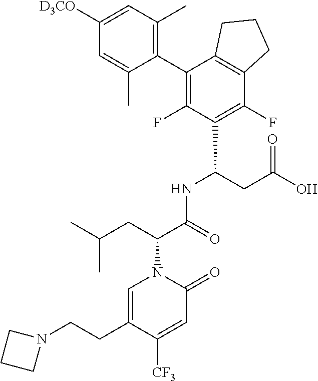 |
| | 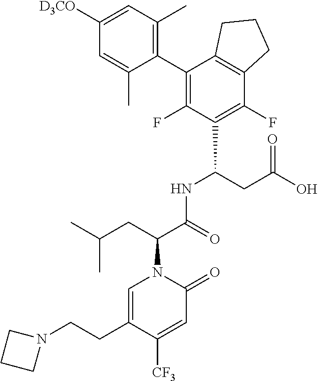 |
| | 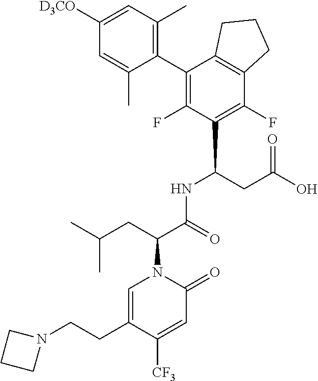 |
| | 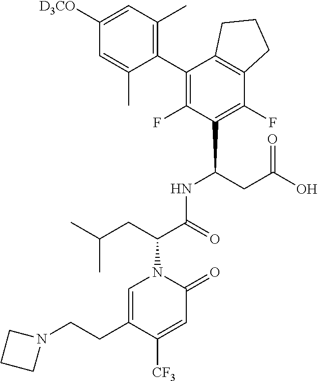 |
| 226 | 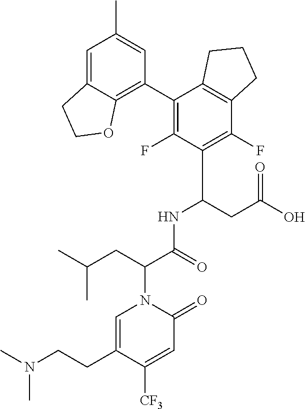 [SMILES: Cc1cc2c(c(-c3c(F)c(C(CC(=O)O)NC(=O)C(CC(C)C)n4cc(CCN(C)C)c(C(F)(F)F)cc4=O)c(F)c4c3CCC4)c1)OCC2] |
| | 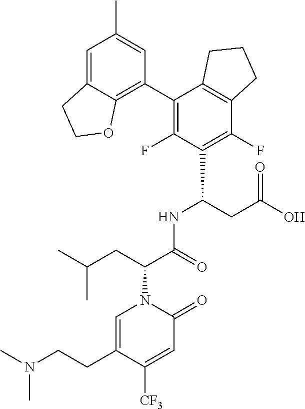 [SMILES: Cc1cc2c(c(-c3c(F)c([C@H](CC(=O)O)NC(=O)[C@@H](CC(C)C)n4cc(CCN(C)C)c(C(F)(F)F)cc4=O)c(F)c4c3CCC4)c1)OCC2] |
| | 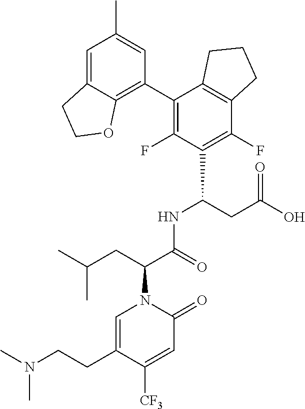 [SMILES: Cc1cc2c(c(-c3c(F)c([C@H](CC(=O)O)NC(=O)[C@H](CC(C)C)n4cc(CCN(C)C)c(C(F)(F)F)cc4=O)c(F)c4c3CCC4)c1)OCC2] |
| | 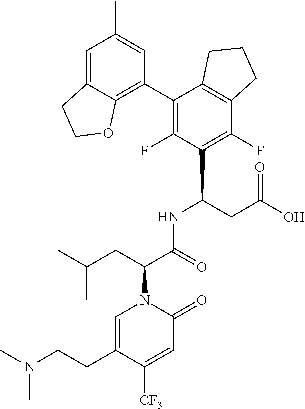 [SMILES: Cc1cc2c(c(-c3c(F)c([C@@H](CC(=O)O)NC(=O)[C@H](CC(C)C)n4cc(CCN(C)C)c(C(F)(F)F)cc4=O)c(F)c4c3CCC4)c1)OCC2] |
| | 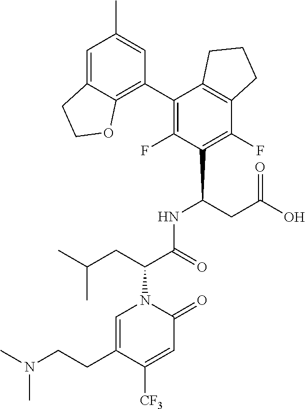 [SMILES: Cc1cc2c(c(-c3c(F)c([C@@H](CC(=O)O)NC(=O)[C@@H](CC(C)C)n4cc(CCN(C)C)c(C(F)(F)F)cc4=O)c(F)c4c3CCC4)c1)OCC2] |

TABLE E-2

| | |
|---|---|
| 227 | 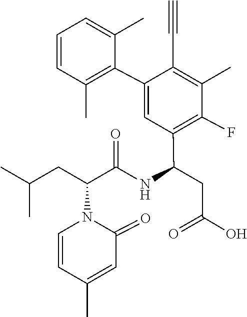 [SMILES: C#Cc1c(-c2c(C)cccc2C)cc([C@@H](CC(=O)O)NC(=O)[C@@H](CC(C)C)n2ccc(C)cc2=O)c(F)c1C] |
| | 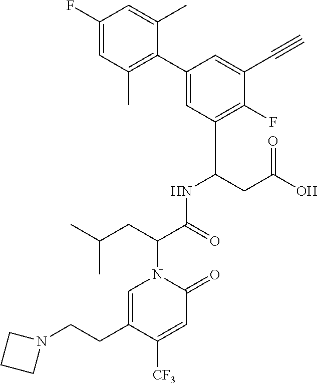 [SMILES: C#Cc1cc(-c2c(C)cc(F)cc2C)cc(C(CC(=O)O)NC(=O)C(CC(C)C)n2cc(CCN3CCC3)c(C(F)(F)F)cc2=O)c1F] |
| | 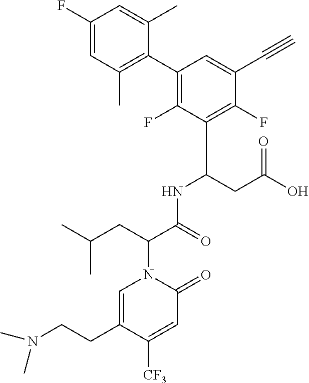 [SMILES: C#Cc1cc(-c2c(C)cc(F)cc2C)c(F)c(C(CC(=O)O)NC(=O)C(CC(C)C)n2cc(CCN(C)C)c(C(F)(F)F)cc2=O)c1F] |
| | 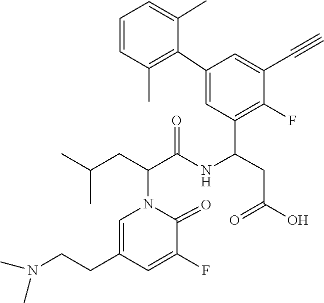 [SMILES: C#Cc1cc(-c2c(C)cccc2C)cc(C(CC(=O)O)NC(=O)C(CC(C)C)n2cc(CCN(C)C)cc(F)c2=O)c1F] |
| | 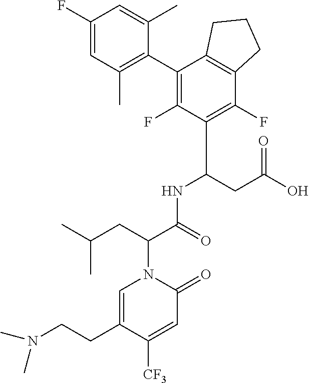 [SMILES: Cc1cc(F)cc(C)c1-c1c(F)c(C(CC(=O)O)NC(=O)C(CC(C)C)n2cc(CCN(C)C)c(C(F)(F)F)cc2=O)c(F)c2c1CCC2] |
| 228 | 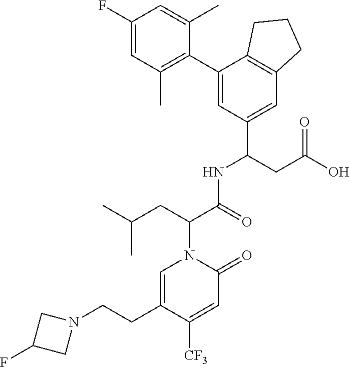 [SMILES: Cc1cc(F)cc(C)c1-c1cc(C(CC(=O)O)NC(=O)C(CC(C)C)n2cc(CCN3CC(F)C3)c(C(F)(F)F)cc2=O)cc2c1CCC2] |
| | 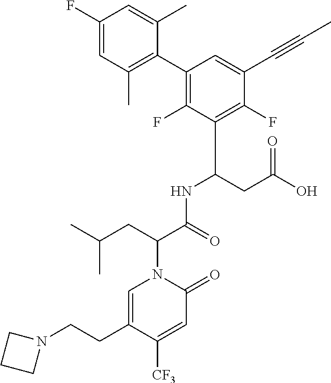 [SMILES: CC#Cc1cc(-c2c(C)cc(F)cc2C)c(F)c(C(CC(=O)O)NC(=O)C(CC(C)C)n2cc(CCN3CCC3)c(C(F)(F)F)cc2=O)c1F] |
| | 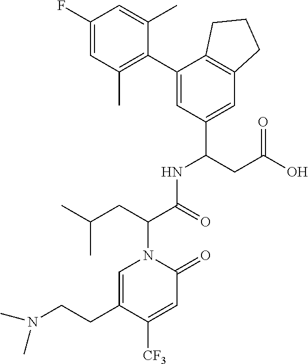 [SMILES: Cc1cc(F)cc(C)c1-c1cc(C(CC(=O)O)NC(=O)C(CC(C)C)n2cc(CCN(C)C)c(C(F)(F)F)cc2=O)cc2c1CCC2] |
| | 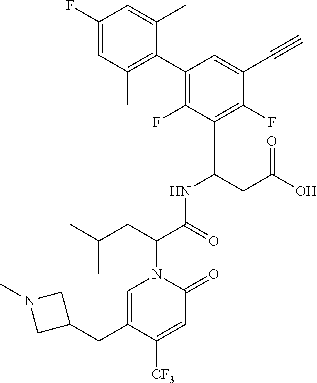 [SMILES: C#Cc1cc(-c2c(C)cc(F)cc2C)c(F)c(C(CC(=O)O)NC(=O)C(CC(C)C)n2cc(CC3CN(C)C3)c(C(F)(F)F)cc2=O)c1F] |
| | 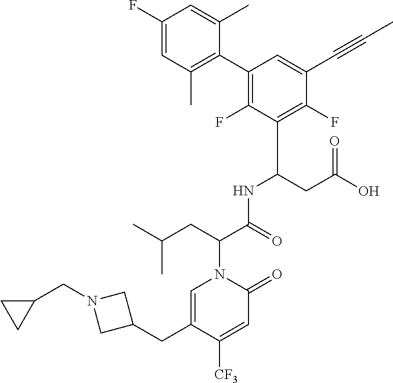 [SMILES: CC#Cc1cc(-c2c(C)cc(F)cc2C)c(F)c(C(CC(=O)O)NC(=O)C(CC(C)C)n2cc(CC3CN(CC4CC4)C3)c(C(F)(F)F)cc2=O)c1F] |
| 229 | 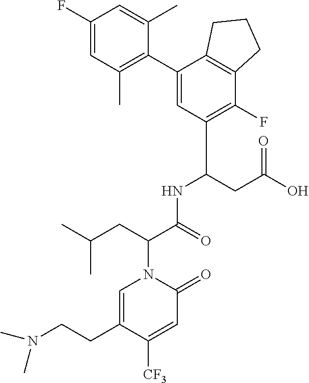 [SMILES: Cc1cc(F)cc(C)c1-c1cc(C(CC(=O)O)NC(=O)C(CC(C)C)n2cc(CCN(C)C)c(C(F)(F)F)cc2=O)c(F)c2c1CCC2] |
| | 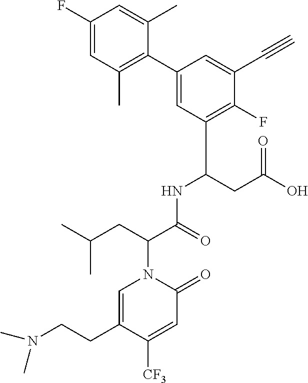 [SMILES: C#Cc1cc(-c2c(C)cc(F)cc2C)cc(C(CC(=O)O)NC(=O)C(CC(C)C)n2cc(CCN(C)C)c(C(F)(F)F)cc2=O)c1F] |
| | 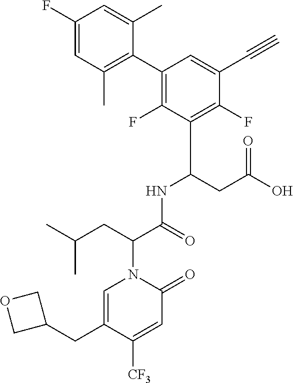 [SMILES: C#Cc1cc(-c2c(C)cc(F)cc2C)c(F)c(C(CC(=O)O)NC(=O)C(CC(C)C)n2cc(CC3COC3)c(C(F)(F)F)cc2=O)c1F] |
| | 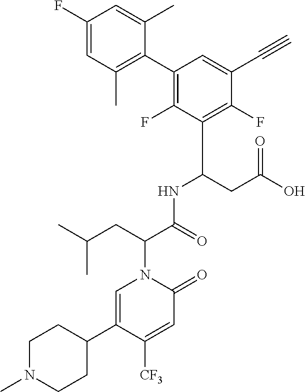 [SMILES: C#Cc1cc(-c2c(C)cc(F)cc2C)c(F)c(C(CC(=O)O)NC(=O)C(CC(C)C)n2cc(C3CCN(C)CC3)c(C(F)(F)F)cc2=O)c1F] |
| | 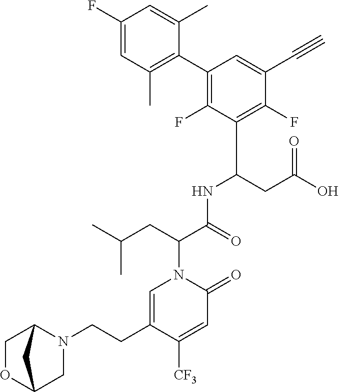 [SMILES: C#Cc1cc(-c2c(C)cc(F)cc2C)c(F)c(C(CC(=O)O)NC(=O)C(CC(C)C)n2cc(CCN3C[C@H]4C[C@@H]3CO4)c(C(F)(F)F)cc2=O)c1F] |
| 230 | 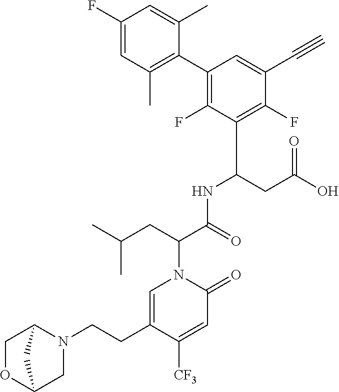 [SMILES: C#Cc1cc(-c2c(C)cc(F)cc2C)c(F)c(C(CC(=O)O)NC(=O)C(CC(C)C)n2cc(CCN3C[C@@H]4C[C@H]3CO4)c(C(F)(F)F)cc2=O)c1F] |
| | 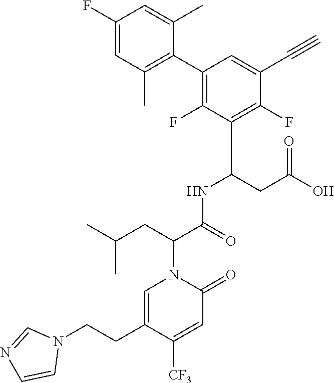 [SMILES: C#Cc1cc(-c2c(C)cc(F)cc2C)c(F)c(C(CC(=O)O)NC(=O)C(CC(C)C)n2cc(CCn3ccnc3)c(C(F)(F)F)cc2=O)c1F] |
| | 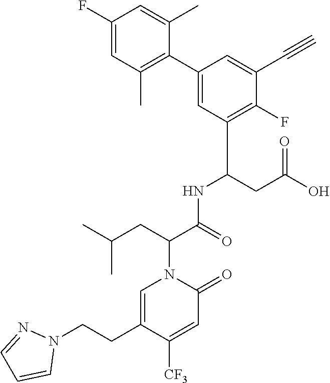 [SMILES: C#Cc1cc(-c2c(C)cc(F)cc2C)cc(C(CC(=O)O)NC(=O)C(CC(C)C)n2cc(CCn3cccn3)c(C(F)(F)F)cc2=O)c1F] |
| | 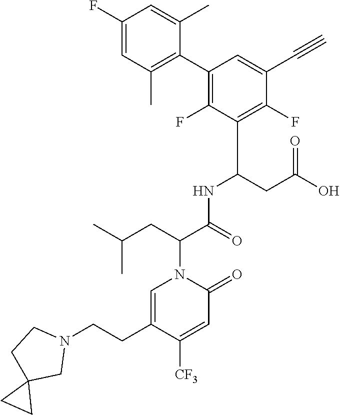 [SMILES: C#Cc1cc(-c2c(C)cc(F)cc2C)c(F)c(C(CC(=O)O)NC(=O)C(CC(C)C)n2cc(CCN3CCC4(CC4)C3)c(C(F)(F)F)cc2=O)c1F] |
| | 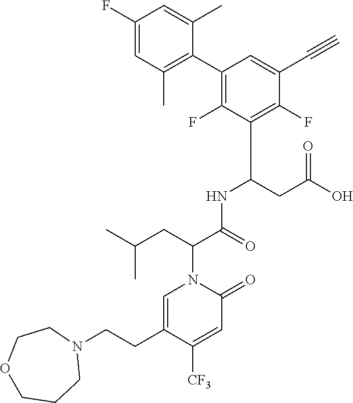 [SMILES: C#Cc1cc(-c2c(C)cc(F)cc2C)c(F)c(C(CC(=O)O)NC(=O)C(CC(C)C)n2cc(CCN3CCCOCC3)c(C(F)(F)F)cc2=O)c1F] |
| 231 | 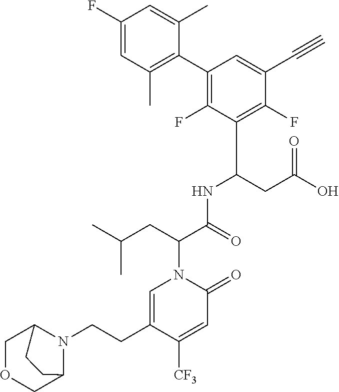 [SMILES: C#Cc1cc(-c2c(C)cc(F)cc2C)c(F)c(C(CC(=O)O)NC(=O)C(CC(C)C)n2cc(CCN3C4CCC3COC4)c(C(F)(F)F)cc2=O)c1F] |
| | 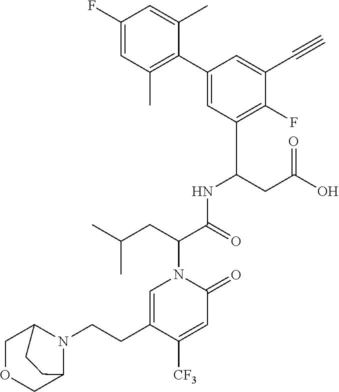 [SMILES: C#Cc1cc(-c2c(C)cc(F)cc2C)cc(C(CC(=O)O)NC(=O)C(CC(C)C)n2cc(CCN3C4CCC3COC4)c(C(F)(F)F)cc2=O)c1F] |
| | 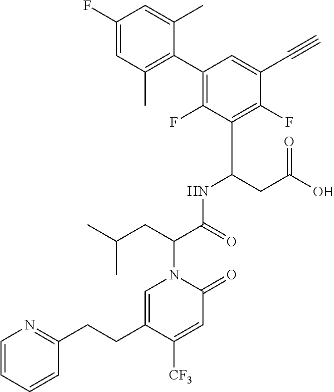 [SMILES: C#Cc1cc(-c2c(C)cc(F)cc2C)c(F)c(C(CC(=O)O)NC(=O)C(CC(C)C)n2cc(CCc3ccccn3)c(C(F)(F)F)cc2=O)c1F] |
| | 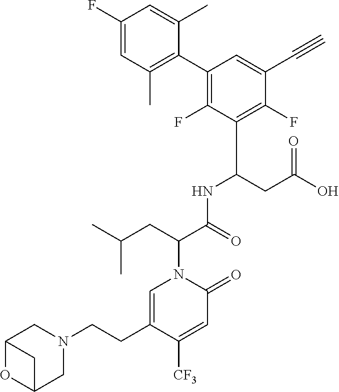 [SMILES: C#Cc1cc(-c2c(C)cc(F)cc2C)c(F)c(C(CC(=O)O)NC(=O)C(CC(C)C)n2cc(CCN3CC4CC(C3)O4)c(C(F)(F)F)cc2=O)c1F] |
| | 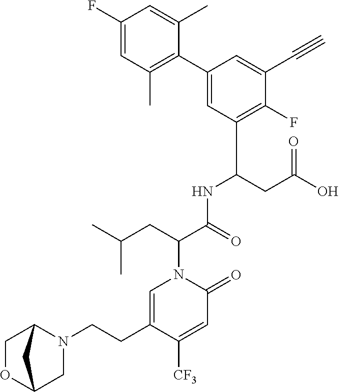 [SMILES: C#Cc1cc(-c2c(C)cc(F)cc2C)cc(C(CC(=O)O)NC(=O)C(CC(C)C)n2cc(CCN3C[C@H]4C[C@@H]3CO4)c(C(F)(F)F)cc2=O)c1F] |
| 232 | 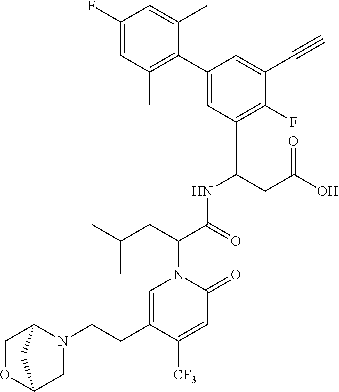 [SMILES: C#Cc1cc(-c2c(C)cc(F)cc2C)cc(C(CC(=O)O)NC(=O)C(CC(C)C)n2cc(CCN3C[C@@H]4C[C@H]3CO4)c(C(F)(F)F)cc2=O)c1F] |
| | 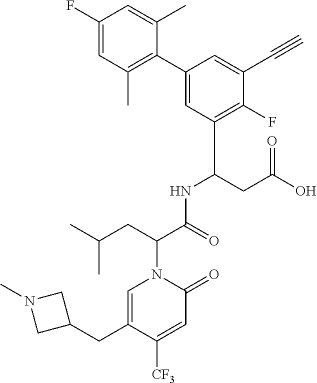 [SMILES: C#Cc1cc(-c2c(C)cc(F)cc2C)cc(C(CC(=O)O)NC(=O)C(CC(C)C)n2cc(CC3CN(C)C3)c(C(F)(F)F)cc2=O)c1F] |
| | 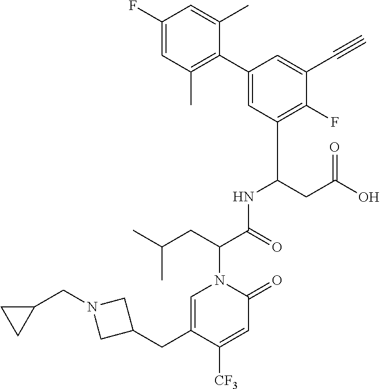 [SMILES: C#Cc1cc(-c2c(C)cc(F)cc2C)cc(C(CC(=O)O)NC(=O)C(CC(C)C)n2cc(CC3CN(CC4CC4)C3)c(C(F)(F)F)cc2=O)c1F] |
| | 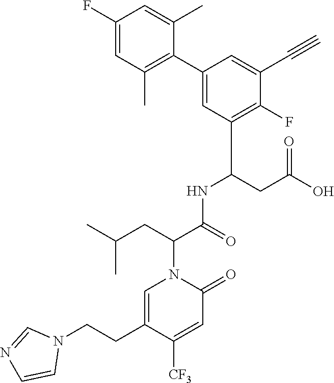 [SMILES: C#Cc1cc(-c2c(C)cc(F)cc2C)cc(C(CC(=O)O)NC(=O)C(CC(C)C)n2cc(CCn3ccnc3)c(C(F)(F)F)cc2=O)c1F] |
| | 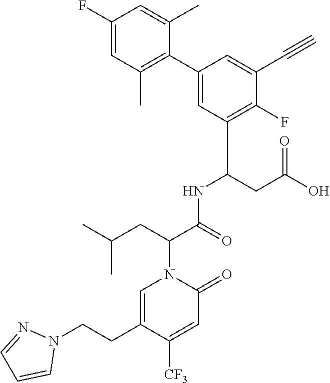 [SMILES: C#Cc1cc(-c2c(C)cc(F)cc2C)cc(C(CC(=O)O)NC(=O)C(CC(C)C)n2cc(CCn3cccn3)c(C(F)(F)F)cc2=O)c1F] |
| 233 | 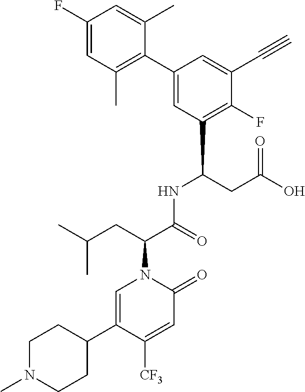 [SMILES: C#Cc1cc(-c2c(C)cc(F)cc2C)cc([C@@H](CC(=O)O)NC(=O)[C@H](CC(C)C)n2cc(C3CCN(C)CC3)c(C(F)(F)F)cc2=O)c1F] |
| | 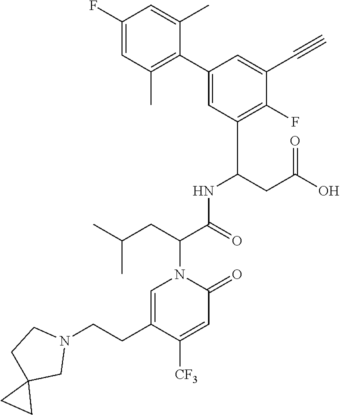 [SMILES: C#Cc1cc(-c2c(C)cc(F)cc2C)cc(C(CC(=O)O)NC(=O)C(CC(C)C)n2cc(CCN3CCC4(CC4)C3)c(C(F)(F)F)cc2=O)c1F] |
| | 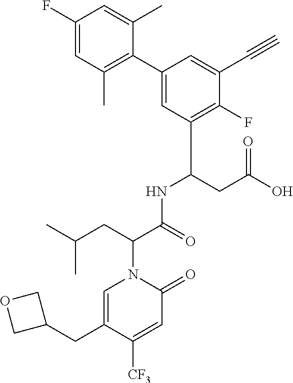 [SMILES: C#Cc1cc(-c2c(C)cc(F)cc2C)cc(C(CC(=O)O)NC(=O)C(CC(C)C)n2cc(CC3COC3)c(C(F)(F)F)cc2=O)c1F] |
| | 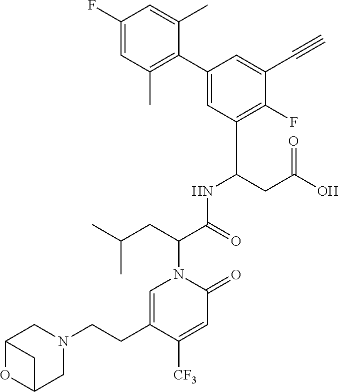 [SMILES: C#Cc1cc(-c2c(C)cc(F)cc2C)cc(C(CC(=O)O)NC(=O)C(CC(C)C)n2cc(CCN3CC4CC(C3)O4)c(C(F)(F)F)cc2=O)c1F] |
| | 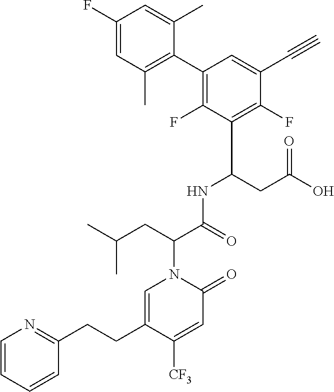 [SMILES: C#Cc1cc(-c2c(C)cc(F)cc2C)c(F)c(C(CC(=O)O)NC(=O)C(CC(C)C)n2cc(CCc3ccccn3)c(C(F)(F)F)cc2=O)c1F] |
| 234 | 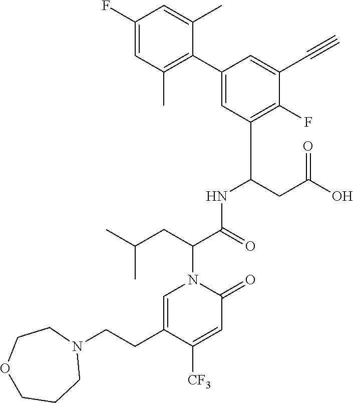 [SMILES: C#Cc1cc(-c2c(C)cc(F)cc2C)cc(C(CC(=O)O)NC(=O)C(CC(C)C)n2cc(CCN3CCCOCC3)c(C(F)(F)F)cc2=O)c1F] |
| | 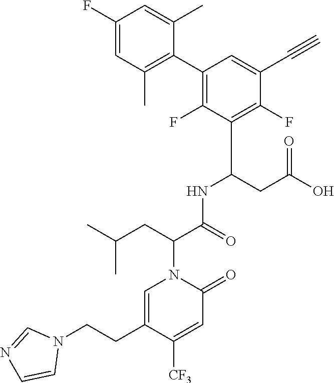 [SMILES: C#Cc1cc(-c2c(C)cc(F)cc2C)c(F)c(C(CC(=O)O)NC(=O)C(CC(C)C)n2cc(CCn3ccnc3)c(C(F)(F)F)cc2=O)c1F] |
| | 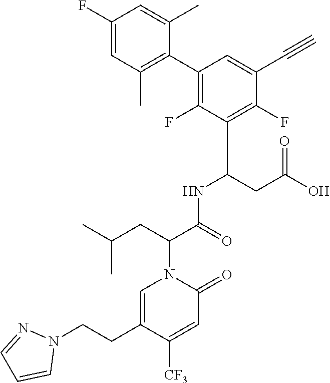 [SMILES: C#Cc1cc(-c2c(C)cc(F)cc2C)c(F)c(C(CC(=O)O)NC(=O)C(CC(C)C)n2cc(CCn3cccn3)c(C(F)(F)F)cc2=O)c1F] |
| | 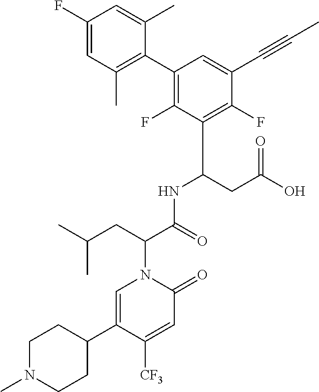 [SMILES: CC#Cc1cc(-c2c(C)cc(F)cc2C)c(F)c(C(CC(=O)O)NC(=O)C(CC(C)C)n2cc(C3CCN(C)CC3)c(C(F)(F)F)cc2=O)c1F] |
| | 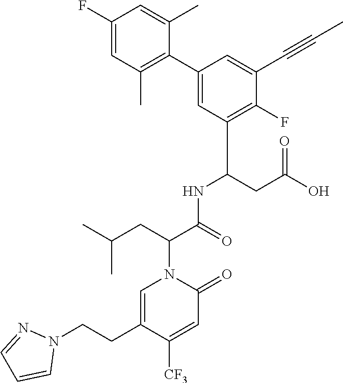 [SMILES: CC#Cc1cc(-c2c(C)cc(F)cc2C)cc(C(CC(=O)O)NC(=O)C(CC(C)C)n2cc(CCn3cccn3)c(C(F)(F)F)cc2=O)c1F] |
| 235 | 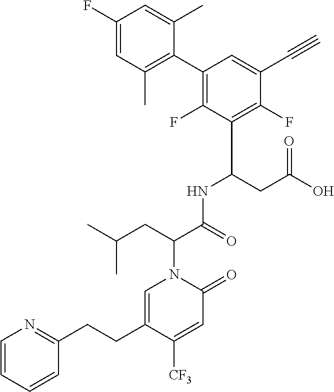 [SMILES: C#Cc1cc(-c2c(C)cc(F)cc2C)c(F)c(C(CC(=O)O)NC(=O)C(CC(C)C)n2cc(CCc3ccccn3)c(C(F)(F)F)cc2=O)c1F] |
| | 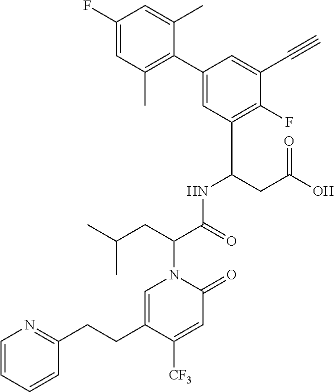 [SMILES: C#Cc1cc(-c2c(C)cc(F)cc2C)cc(C(CC(=O)O)NC(=O)C(CC(C)C)n2cc(CCc3ccccn3)c(C(F)(F)F)cc2=O)c1F] |
| | 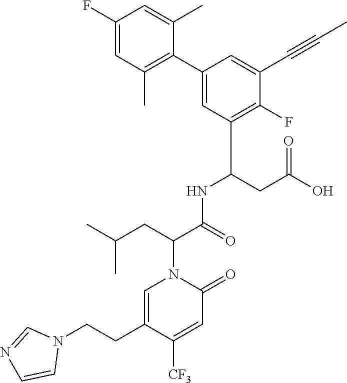 [SMILES: CC#Cc1cc(-c2c(C)cc(F)cc2C)cc(C(CC(=O)O)NC(=O)C(CC(C)C)n2cc(CCn3ccnc3)c(C(F)(F)F)cc2=O)c1F] |
| | 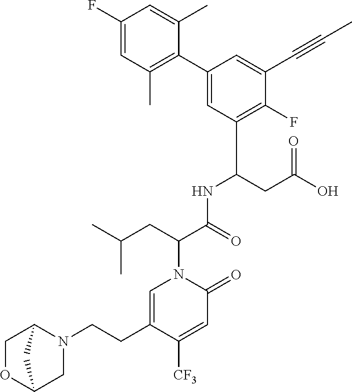 [SMILES: CC#Cc1cc(-c2c(C)cc(F)cc2C)cc(C(CC(=O)O)NC(=O)C(CC(C)C)n2cc(CCN3C[C@@H]4C[C@H]3CO4)c(C(F)(F)F)cc2=O)c1F] |
| | 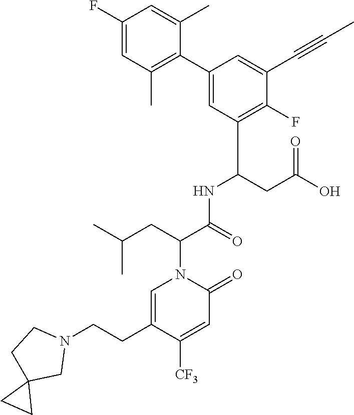 [SMILES: CC#Cc1cc(-c2c(C)cc(F)cc2C)cc(C(CC(=O)O)NC(=O)C(CC(C)C)n2cc(CCN3CCC4(CC4)C3)c(C(F)(F)F)cc2=O)c1F] |
| 236 | 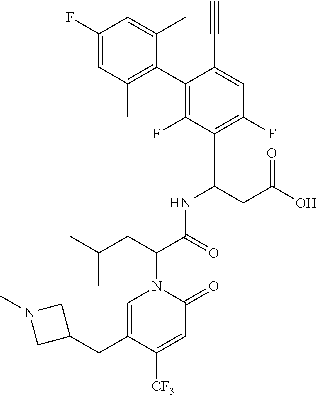 [SMILES: C#Cc1cc(F)c(C(CC(=O)O)NC(=O)C(CC(C)C)n2cc(CC3CN(C)C3)c(C(F)(F)F)cc2=O)c(F)c1-c1c(C)cc(F)cc1C] |
| | 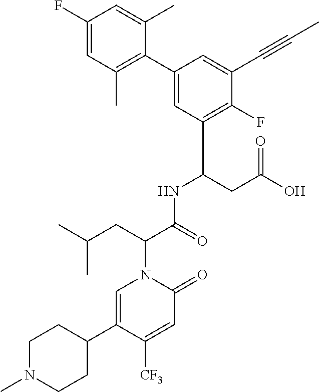 [SMILES: CC#Cc1cc(-c2c(C)cc(F)cc2C)cc(C(CC(=O)O)NC(=O)C(CC(C)C)n2cc(C3CCN(C)CC3)c(C(F)(F)F)cc2=O)c1F] |
| | 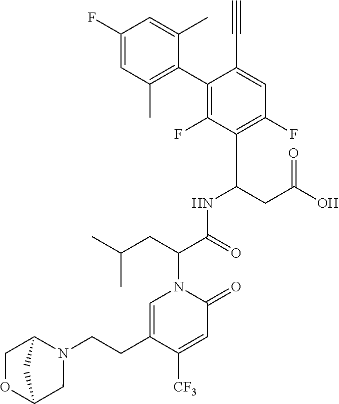 [SMILES: C#Cc1cc(F)c(C(CC(=O)O)NC(=O)C(CC(C)C)n2cc(CCN3C[C@@H]4C[C@H]3CO4)c(C(F)(F)F)cc2=O)c(F)c1-c1c(C)cc(F)cc1C] |
| | 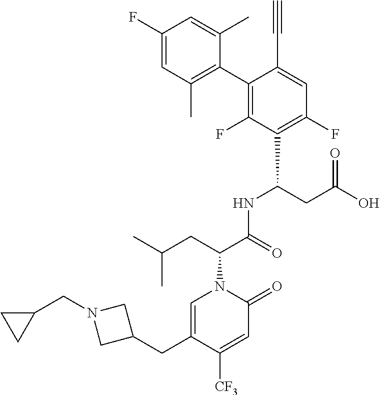 [SMILES: C#Cc1cc(F)c([C@H](CC(=O)O)NC(=O)[C@@H](CC(C)C)n2cc(CC3CN(CC4CC4)C3)c(C(F)(F)F)cc2=O)c(F)c1-c1c(C)cc(F)cc1C] |
| | 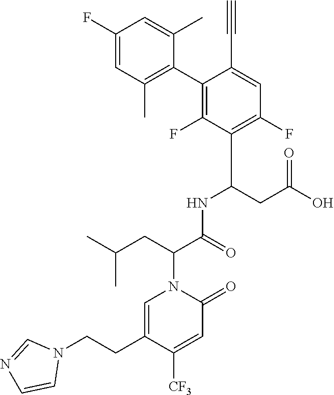 [SMILES: C#Cc1cc(F)c(C(CC(=O)O)NC(=O)C(CC(C)C)n2cc(CCn3ccnc3)c(C(F)(F)F)cc2=O)c(F)c1-c1c(C)cc(F)cc1C] |
| 237 | 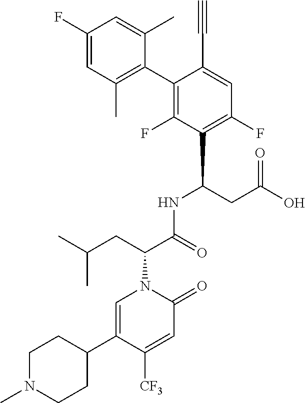 [SMILES: C#Cc1cc(F)c([C@@H](CC(=O)O)NC(=O)[C@@H](CC(C)C)n2cc(C3CCN(C)CC3)c(C(F)(F)F)cc2=O)c(F)c1-c1c(C)cc(F)cc1C] |
| | 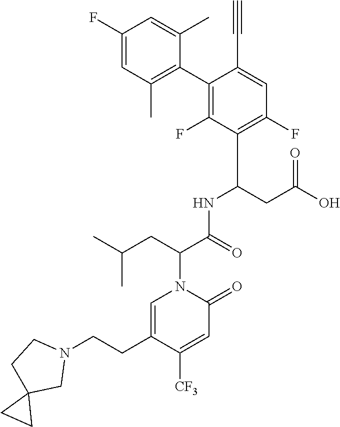 [SMILES: C#Cc1cc(F)c(C(CC(=O)O)NC(=O)C(CC(C)C)n2cc(CCN3CCC4(CC4)C3)c(C(F)(F)F)cc2=O)c(F)c1-c1c(C)cc(F)cc1C] |
| | 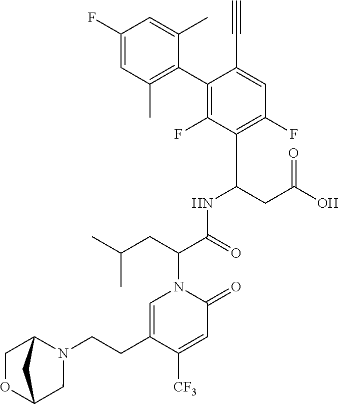 [SMILES: C#Cc1cc(F)c(C(CC(=O)O)NC(=O)C(CC(C)C)n2cc(CCN3C[C@H]4C[C@@H]3CO4)c(C(F)(F)F)cc2=O)c(F)c1-c1c(C)cc(F)cc1C] |
| | 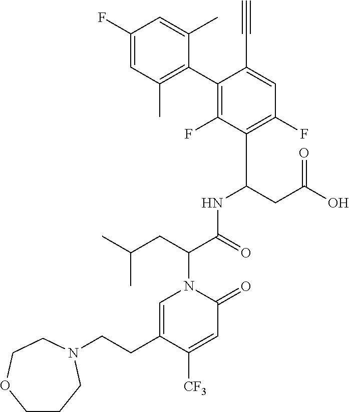 [SMILES: C#Cc1cc(F)c(C(CC(=O)O)NC(=O)C(CC(C)C)n2cc(CCN3CCCOCC3)c(C(F)(F)F)cc2=O)c(F)c1-c1c(C)cc(F)cc1C] |
| | 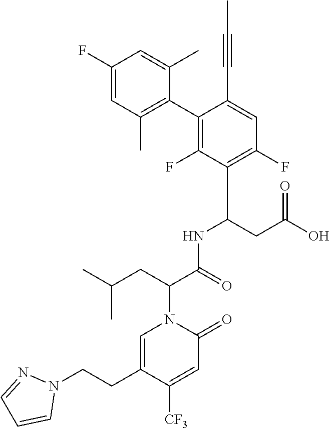 [SMILES: CC#Cc1cc(F)c(C(CC(=O)O)NC(=O)C(CC(C)C)n2cc(CCn3cccn3)c(C(F)(F)F)cc2=O)c(F)c1-c1c(C)cc(F)cc1C] |
| 238 | 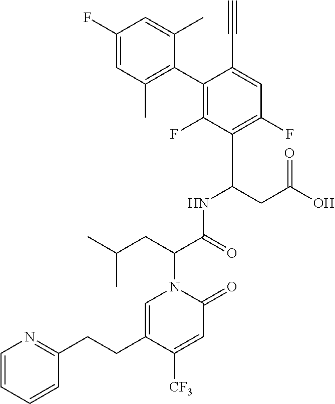 [SMILES: C#Cc1cc(F)c(C(CC(=O)O)NC(=O)C(CC(C)C)n2cc(CCc3ccccn3)c(C(F)(F)F)cc2=O)c(F)c1-c1c(C)cc(F)cc1C] |
| | 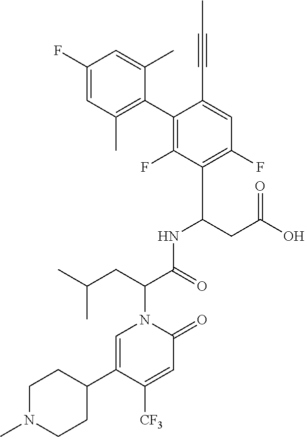 [SMILES: CC#Cc1cc(F)c(C(CC(=O)O)NC(=O)C(CC(C)C)n2cc(C3CCN(C)CC3)c(C(F)(F)F)cc2=O)c(F)c1-c1c(C)cc(F)cc1C] |
| | 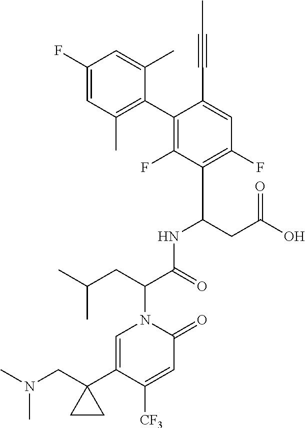 [SMILES: CC#Cc1cc(F)c(C(CC(=O)O)NC(=O)C(CC(C)C)n2cc(C3(CN(C)C)CC3)c(C(F)(F)F)cc2=O)c(F)c1-c1c(C)cc(F)cc1C] |
| | 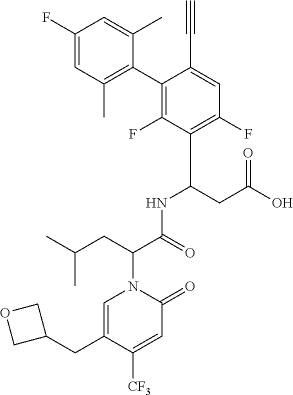 [SMILES: C#Cc1cc(F)c(C(CC(=O)O)NC(=O)C(CC(C)C)n2cc(CC3COC3)c(C(F)(F)F)cc2=O)c(F)c1-c1c(C)cc(F)cc1C] |
| | 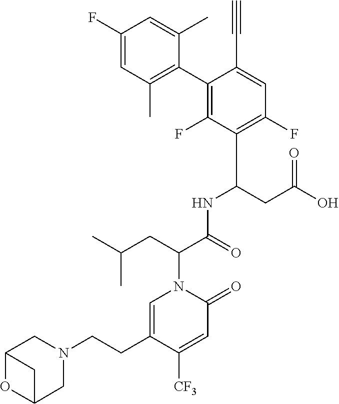 [SMILES: C#Cc1cc(F)c(C(CC(=O)O)NC(=O)C(CC(C)C)n2cc(CCN3CC4CC(C3)O4)c(C(F)(F)F)cc2=O)c(F)c1-c1c(C)cc(F)cc1C] |
| 239 | 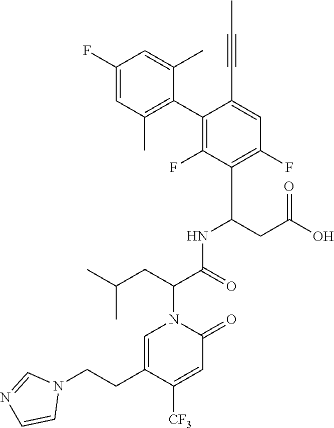 [SMILES: CC#Cc1cc(F)c(C(CC(=O)O)NC(=O)C(CC(C)C)n2cc(CCn3ccnc3)c(C(F)(F)F)cc2=O)c(F)c1-c1c(C)cc(F)cc1C] |
| | 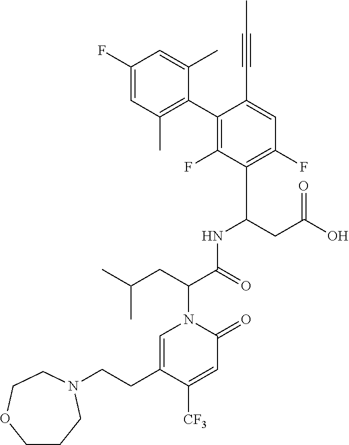 [SMILES: CC#Cc1cc(F)c(C(CC(=O)O)NC(=O)C(CC(C)C)n2cc(CCN3CCCOCC3)c(C(F)(F)F)cc2=O)c(F)c1-c1c(C)cc(F)cc1C] |
| | 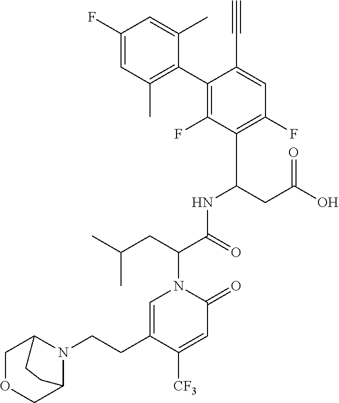 [SMILES: C#Cc1cc(F)c(C(CC(=O)O)NC(=O)C(CC(C)C)n2cc(CCN3C4CCC3COC4)c(C(F)(F)F)cc2=O)c(F)c1-c1c(C)cc(F)cc1C] |
| | 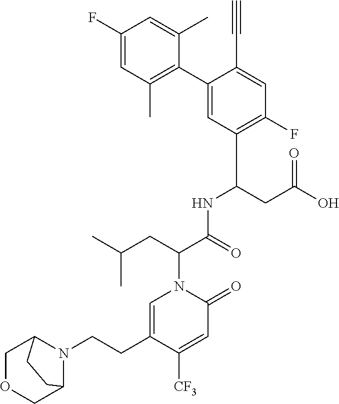 [SMILES: C#Cc1cc(F)c(C(CC(=O)O)NC(=O)C(CC(C)C)n2cc(CCN3C4CCC3COC4)c(C(F)(F)F)cc2=O)cc1-c1c(C)cc(F)cc1C] |
| | 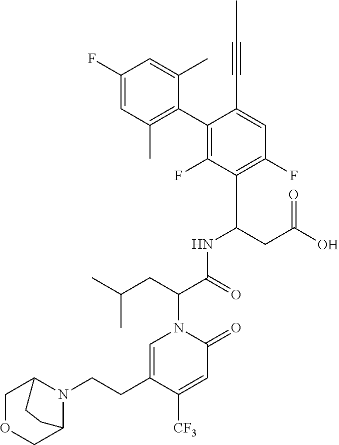 [SMILES: CC#Cc1cc(F)c(C(CC(=O)O)NC(=O)C(CC(C)C)n2cc(CCN3C4CCC3COC4)c(C(F)(F)F)cc2=O)c(F)c1-c1c(C)cc(F)cc1C] |
| 240 | 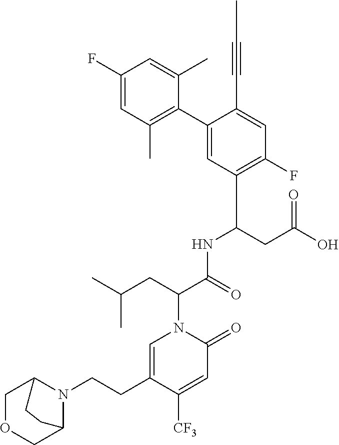 [SMILES: CC#Cc1cc(F)c(C(CC(=O)O)NC(=O)C(CC(C)C)n2cc(CCN3C4CCC3COC4)c(C(F)(F)F)cc2=O)cc1-c1c(C)cc(F)cc1C] |
| | 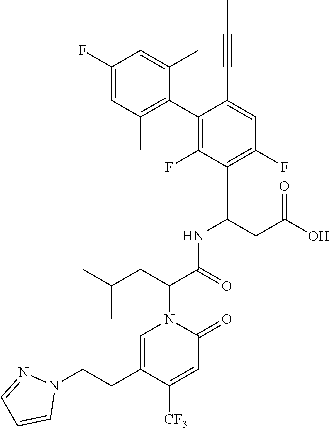 [SMILES: CC#Cc1cc(F)c(C(CC(=O)O)NC(=O)C(CC(C)C)n2cc(CCn3cccn3)c(C(F)(F)F)cc2=O)c(F)c1-c1c(C)cc(F)cc1C] |
| | 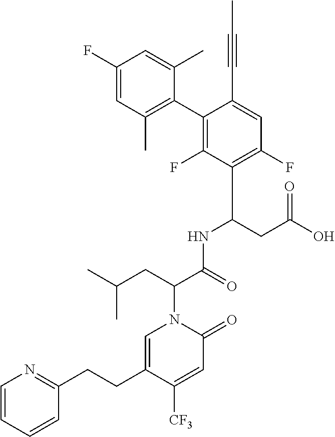 [SMILES: CC#Cc1cc(F)c(C(CC(=O)O)NC(=O)C(CC(C)C)n2cc(CCc3ccccn3)c(C(F)(F)F)cc2=O)c(F)c1-c1c(C)cc(F)cc1C] |
| | 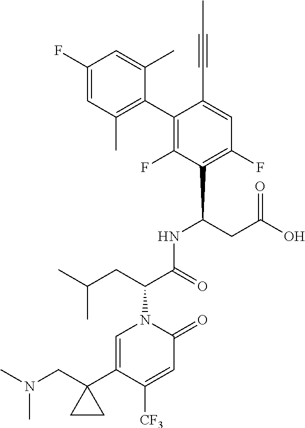 [SMILES: CC#Cc1cc(F)c([C@@H](CC(=O)O)NC(=O)[C@@H](CC(C)C)n2cc(C3(CN(C)C)CC3)c(C(F)(F)F)cc2=O)c(F)c1-c1c(C)cc(F)cc1C] |
| | 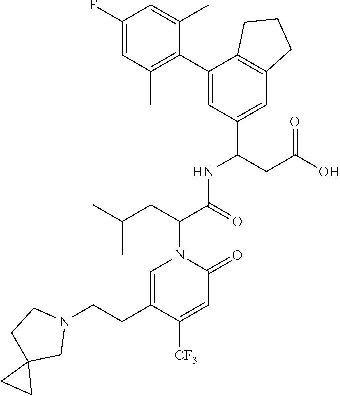 [SMILES: Cc1cc(F)cc(C)c1-c1cc(C(CC(=O)O)NC(=O)C(CC(C)C)n2cc(CCN3CCC4(CC4)C3)c(C(F)(F)F)cc2=O)cc2c1CCC2] |
| 241 | 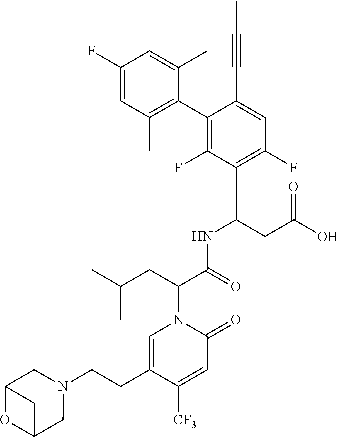 [SMILES: CC#Cc1cc(F)c(C(CC(=O)O)NC(=O)C(CC(C)C)n2cc(CCN3CC4CC(C3)O4)c(C(F)(F)F)cc2=O)c(F)c1-c1c(C)cc(F)cc1C] |
| | 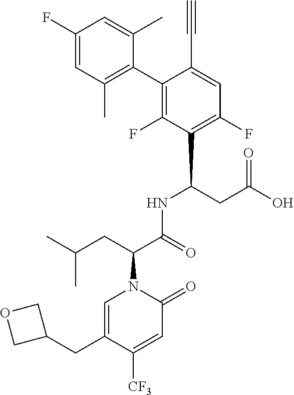 [SMILES: C#Cc1cc(F)c([C@@H](CC(=O)O)NC(=O)[C@H](CC(C)C)n2cc(CC3COC3)c(C(F)(F)F)cc2=O)c(F)c1-c1c(C)cc(F)cc1C] |
| | 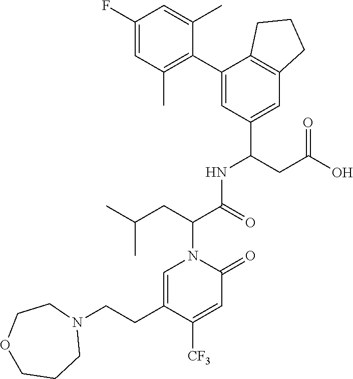 [SMILES: Cc1cc(F)cc(C)c1-c1cc(C(CC(=O)O)NC(=O)C(CC(C)C)n2cc(CCN3CCCOCC3)c(C(F)(F)F)cc2=O)cc2c1CCC2] |
| | 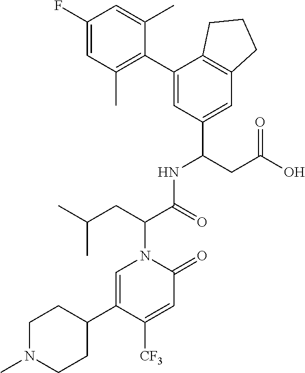 [SMILES: Cc1cc(F)cc(C)c1-c1cc(C(CC(=O)O)NC(=O)C(CC(C)C)n2cc(C3CCN(C)CC3)c(C(F)(F)F)cc2=O)cc2c1CCC2] |
| | 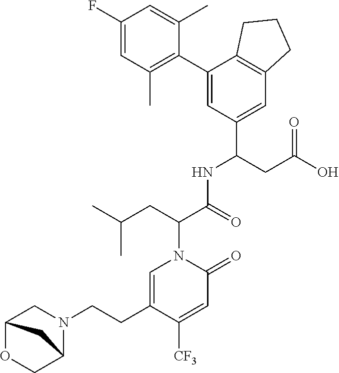 [SMILES: Cc1cc(F)cc(C)c1-c1cc(C(CC(=O)O)NC(=O)C(CC(C)C)n2cc(CCN3C[C@@H]4C[C@H]3CO4)c(C(F)(F)F)cc2=O)cc2c1CCC2] |
| 242 | 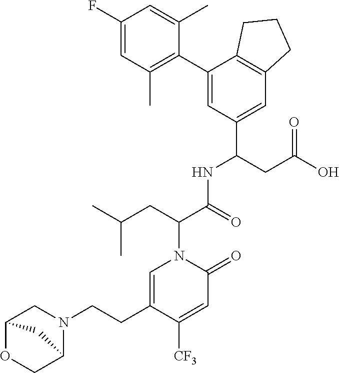 [SMILES: Cc1cc(F)cc(C)c1-c1cc(C(CC(=O)O)NC(=O)C(CC(C)C)n2cc(CCN3C[C@H]4C[C@@H]3CO4)c(C(F)(F)F)cc2=O)cc2c1CCC2] |
| | 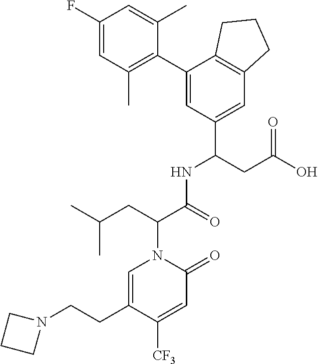 [SMILES: Cc1cc(F)cc(C)c1-c1cc(C(CC(=O)O)NC(=O)C(CC(C)C)n2cc(CCN3CCC3)c(C(F)(F)F)cc2=O)cc2c1CCC2] |
| | 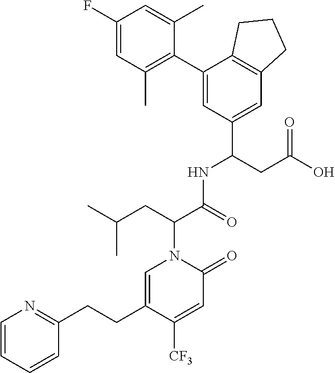 [SMILES: Cc1cc(F)cc(C)c1-c1cc(C(CC(=O)O)NC(=O)C(CC(C)C)n2cc(CCc3ccccn3)c(C(F)(F)F)cc2=O)cc2c1CCC2] |
| | 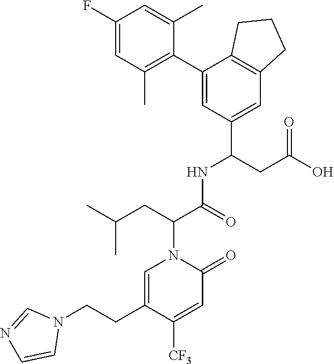 [SMILES: Cc1cc(F)cc(C)c1-c1cc(C(CC(=O)O)NC(=O)C(CC(C)C)n2cc(CCn3ccnc3)c(C(F)(F)F)cc2=O)cc2c1CCC2] |
| | 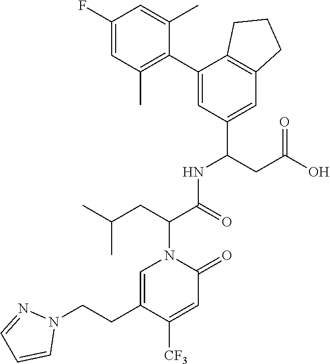 [SMILES: Cc1cc(F)cc(C)c1-c1cc(C(CC(=O)O)NC(=O)C(CC(C)C)n2cc(CCn3cccn3)c(C(F)(F)F)cc2=O)cc2c1CCC2] |
| 243 | 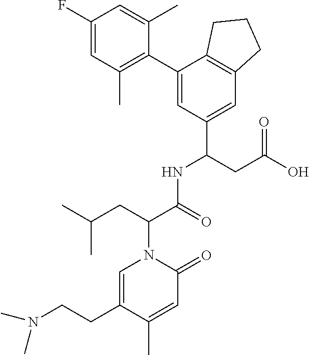 [SMILES: Cc1cc(=O)n(C(CC(C)C)C(=O)NC(CC(=O)O)c2cc3c(c(-c4c(C)cc(F)cc4C)c2)CCC3)cc1CCN(C)C] |
| | 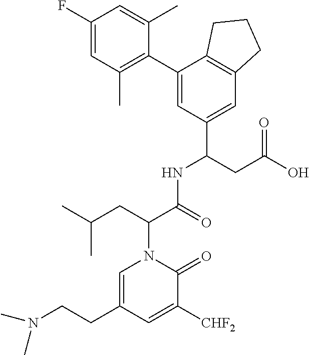 [SMILES: Cc1cc(F)cc(C)c1-c1cc(C(CC(=O)O)NC(=O)C(CC(C)C)n2cc(CCN(C)C)cc(C(F)F)c2=O)cc2c1CCC2] |
| | 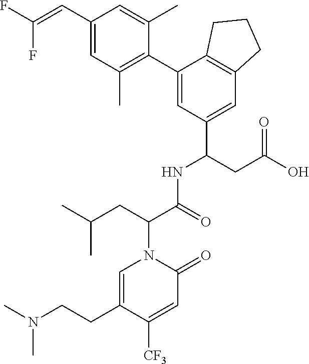 [SMILES: Cc1cc(C=C(F)F)cc(C)c1-c1cc(C(CC(=O)O)NC(=O)C(CC(C)C)n2cc(CCN(C)C)c(C(F)(F)F)cc2=O)cc2c1CCC2] |
| | 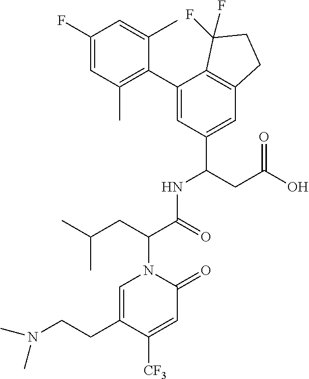 [SMILES: Cc1cc(F)cc(C)c1-c1cc(C(CC(=O)O)NC(=O)C(CC(C)C)n2cc(CCN(C)C)c(C(F)(F)F)cc2=O)cc2c1C(F)(F)CC2] |
| | 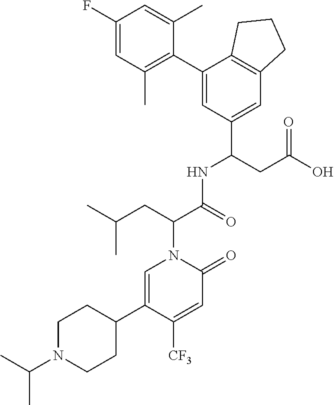 [SMILES: Cc1cc(F)cc(C)c1-c1cc(C(CC(=O)O)NC(=O)C(CC(C)C)n2cc(C3CCN(C(C)C)CC3)c(C(F)(F)F)cc2=O)cc2c1CCC2] |
| 244 | 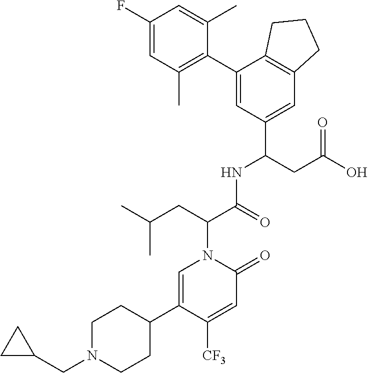 [SMILES: Cc1cc(F)cc(C)c1-c1cc(C(CC(=O)O)NC(=O)C(CC(C)C)n2cc(C3CCN(CC4CC4)CC3)c(C(F)(F)F)cc2=O)cc2c1CCC2] |
| | 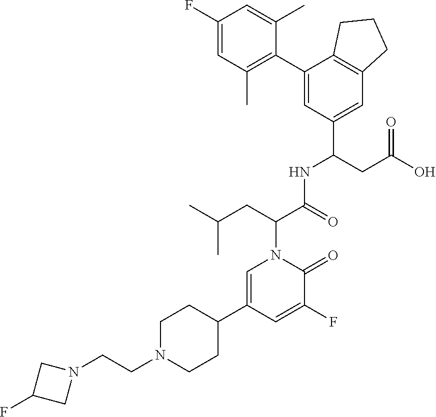 [SMILES: Cc1cc(F)cc(C)c1-c1cc(C(CC(=O)O)NC(=O)C(CC(C)C)n2cc(C3CCN(CCN4CC(F)C4)CC3)cc(F)c2=O)cc2c1CCC2] |
| | 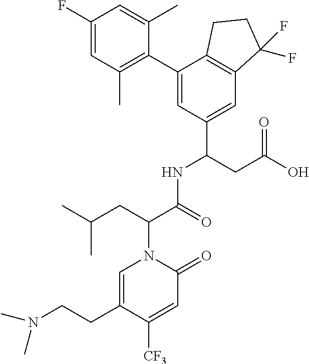 [SMILES: Cc1cc(F)cc(C)c1-c1cc(C(CC(=O)O)NC(=O)C(CC(C)C)n2cc(CCN(C)C)c(C(F)(F)F)cc2=O)cc2c1CCC2(F)F] |
| | 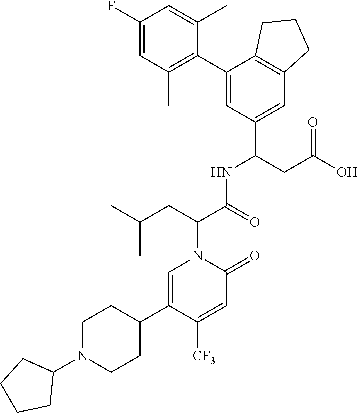 [SMILES: Cc1cc(F)cc(C)c1-c1cc(C(CC(=O)O)NC(=O)C(CC(C)C)n2cc(C3CCN(C4CCCC4)CC3)c(C(F)(F)F)cc2=O)cc2c1CCC2] |
| | 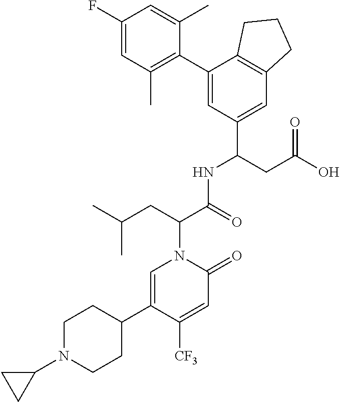 [SMILES: Cc1cc(F)cc(C)c1-c1cc(C(CC(=O)O)NC(=O)C(CC(C)C)n2cc(C3CCN(C4CC4)CC3)c(C(F)(F)F)cc2=O)cc2c1CCC2] |
| 245 | 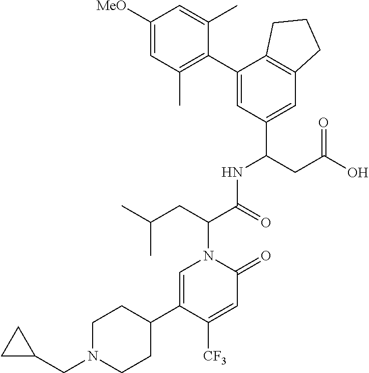 [SMILES: COc1cc(C)c(-c2cc(C(CC(=O)O)NC(=O)C(CC(C)C)n3cc(C4CCN(CC5CC5)CC4)c(C(F)(F)F)cc3=O)cc3c2CCC3)c(C)c1] |
| | 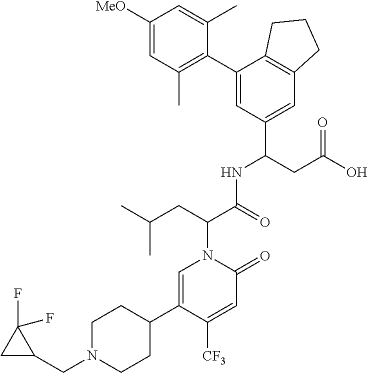 [SMILES: COc1cc(C)c(-c2cc(C(CC(=O)O)NC(=O)C(CC(C)C)n3cc(C4CCN(CC5CC5(F)F)CC4)c(C(F)(F)F)cc3=O)cc3c2CCC3)c(C)c1] |
| | 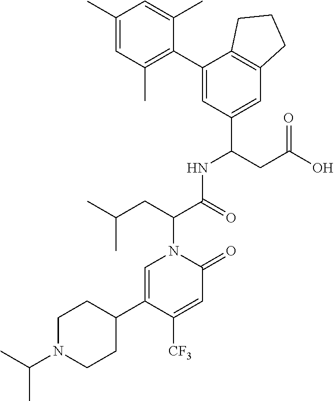 [SMILES: Cc1cc(C)c(-c2cc(C(CC(=O)O)NC(=O)C(CC(C)C)n3cc(C4CCN(C(C)C)CC4)c(C(F)(F)F)cc3=O)cc3c2CCC3)c(C)c1] |
| | 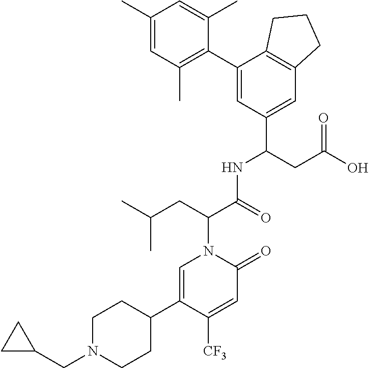 [SMILES: Cc1cc(C)c(-c2cc(C(CC(=O)O)NC(=O)C(CC(C)C)n3cc(C4CCN(CC5CC5)CC4)c(C(F)(F)F)cc3=O)cc3c2CCC3)c(C)c1] |
| | 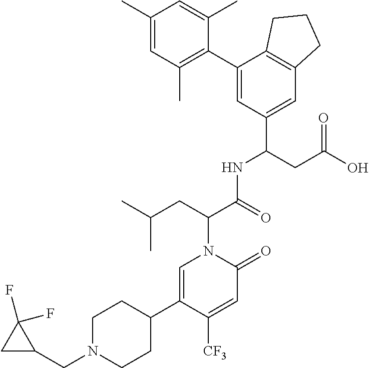 [SMILES: Cc1cc(C)c(-c2cc(C(CC(=O)O)NC(=O)C(CC(C)C)n3cc(C4CCN(CC5CC5(F)F)CC4)c(C(F)(F)F)cc3=O)cc3c2CCC3)c(C)c1] |
| 246 | 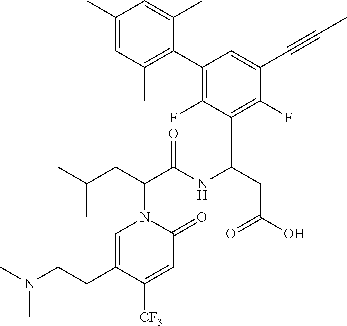 [SMILES: CC#Cc1cc(-c2c(C)cc(C)cc2C)c(F)c(C(CC(=O)O)NC(=O)C(CC(C)C)n2cc(CCN(C)C)c(C(F)(F)F)cc2=O)c1F] |
| | 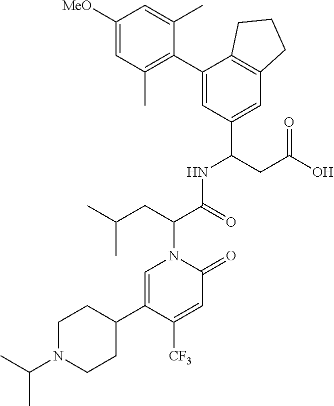 [SMILES: COc1cc(C)c(-c2cc(C(CC(=O)O)NC(=O)C(CC(C)C)n3cc(C4CCN(C(C)C)CC4)c(C(F)(F)F)cc3=O)cc3c2CCC3)c(C)c1] |
| | 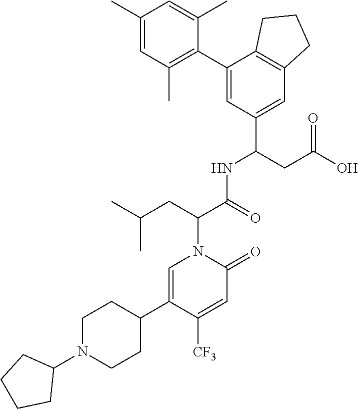 [SMILES: Cc1cc(C)c(-c2cc(C(CC(=O)O)NC(=O)C(CC(C)C)n3cc(C4CCN(C5CCCC5)CC4)c(C(F)(F)F)cc3=O)cc3c2CCC3)c(C)c1] |
| | 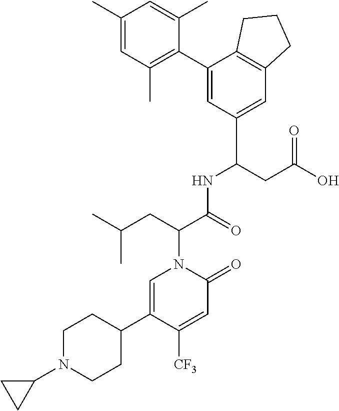 [SMILES: Cc1cc(C)c(-c2cc(C(CC(=O)O)NC(=O)C(CC(C)C)n3cc(C4CCN(C5CC5)CC4)c(C(F)(F)F)cc3=O)cc3c2CCC3)c(C)c1] |
| | 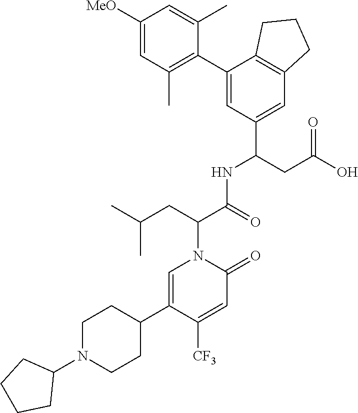 [SMILES: COc1cc(C)c(-c2cc(C(CC(=O)O)NC(=O)C(CC(C)C)n3cc(C4CCN(C5CCCC5)CC4)c(C(F)(F)F)cc3=O)cc3c2CCC3)c(C)c1] |
| 247 | 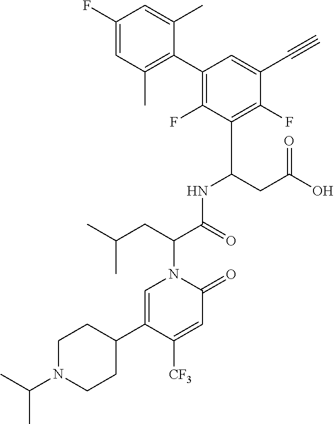 [SMILES: C#Cc1cc(-c2c(C)cc(F)cc2C)c(F)c(C(CC(=O)O)NC(=O)C(CC(C)C)n2cc(C3CCN(C(C)C)CC3)c(C(F)(F)F)cc2=O)c1F] |
| | 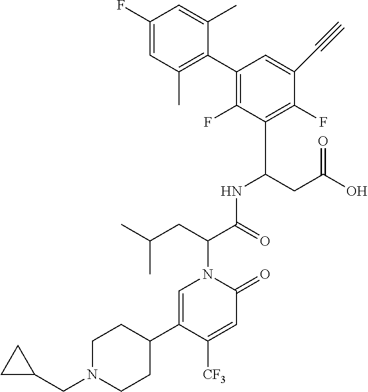 [SMILES: C#Cc1cc(-c2c(C)cc(F)cc2C)c(F)c(C(CC(=O)O)NC(=O)C(CC(C)C)n2cc(C3CCN(CC4CC4)CC3)c(C(F)(F)F)cc2=O)c1F] |
| | 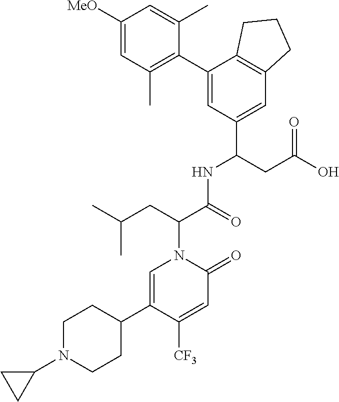 [SMILES: COc1cc(C)c(-c2cc(C(CC(=O)O)NC(=O)C(CC(C)C)n3cc(C4CCN(C5CC5)CC4)c(C(F)(F)F)cc3=O)cc3c2CCC3)c(C)c1] |
| | 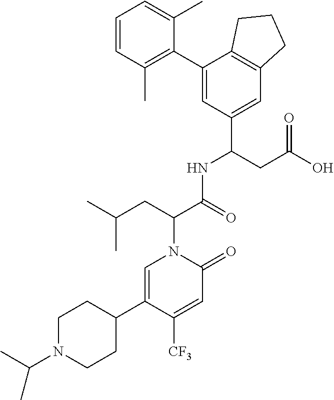 [SMILES: Cc1cccc(C)c1-c1cc(C(CC(=O)O)NC(=O)C(CC(C)C)n2cc(C3CCN(C(C)C)CC3)c(C(F)(F)F)cc2=O)cc2c1CCC2] |
| | 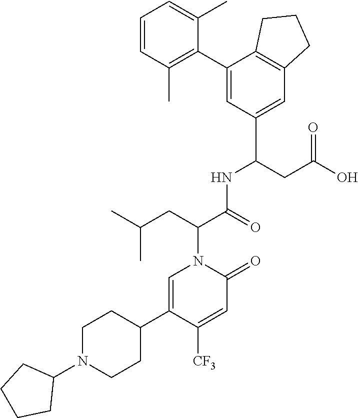 [SMILES: Cc1cccc(C)c1-c1cc(C(CC(=O)O)NC(=O)C(CC(C)C)n2cc(C3CCN(C4CCCC4)CC3)c(C(F)(F)F)cc2=O)cc2c1CCC2] |
| 248 | 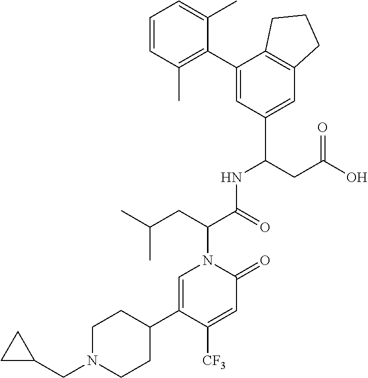 [SMILES: Cc1cccc(C)c1-c1cc(C(CC(=O)O)NC(=O)C(CC(C)C)n2cc(C3CCN(CC4CC4)CC3)c(C(F)(F)F)cc2=O)cc2c1CCC2] |
| | 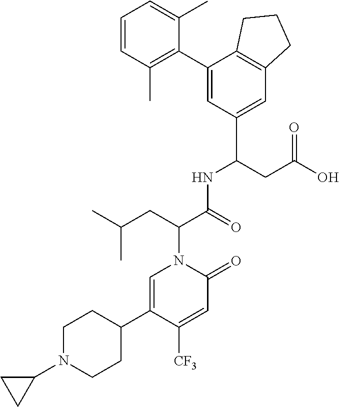 [SMILES: Cc1cccc(C)c1-c1cc(C(CC(=O)O)NC(=O)C(CC(C)C)n2cc(C3CCN(C4CC4)CC3)c(C(F)(F)F)cc2=O)cc2c1CCC2] |
| | 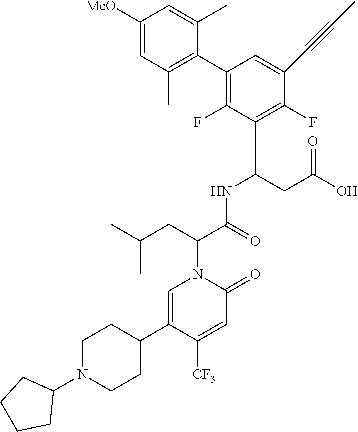 [SMILES: CC#Cc1cc(-c2c(C)cc(OC)cc2C)c(F)c(C(CC(=O)O)NC(=O)C(CC(C)C)n2cc(C3CCN(C4CCCC4)CC3)c(C(F)(F)F)cc2=O)c1F] |
| | 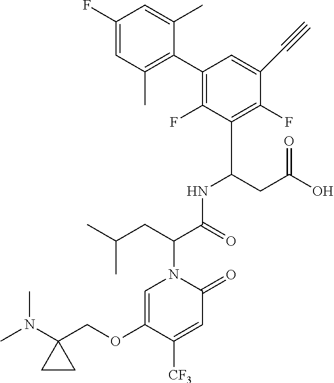 [SMILES: C#Cc1cc(-c2c(C)cc(F)cc2C)c(F)c(C(CC(=O)O)NC(=O)C(CC(C)C)n2cc(OCC3(N(C)C)CC3)c(C(F)(F)F)cc2=O)c1F] |
| | 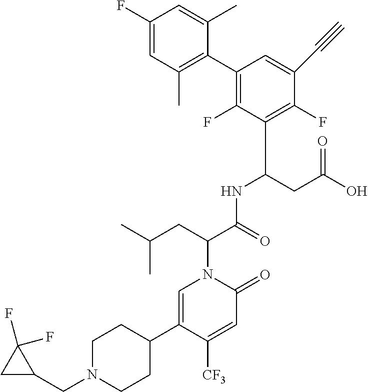 [SMILES: C#Cc1cc(-c2c(C)cc(F)cc2C)c(F)c(C(CC(=O)O)NC(=O)C(CC(C)C)n2cc(C3CCN(CC4CC4(F)F)CC3)c(C(F)(F)F)cc2=O)c1F] |
| 249 | 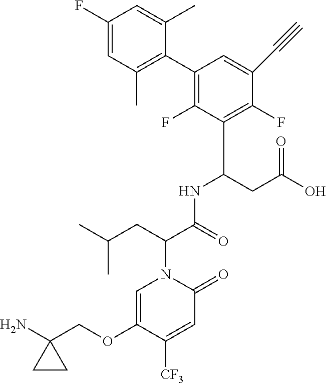 [SMILES: C#Cc1cc(-c2c(C)cc(F)cc2C)c(F)c(C(CC(=O)O)NC(=O)C(CC(C)C)n2cc(OCC3(N)CC3)c(C(F)(F)F)cc2=O)c1F] |
| | 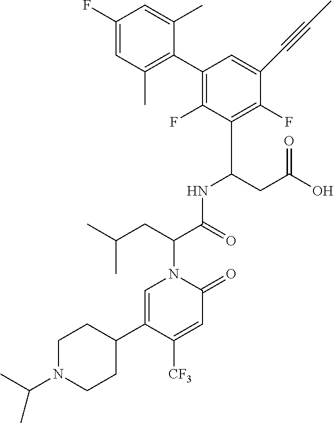 [SMILES: CC#Cc1cc(-c2c(C)cc(F)cc2C)c(F)c(C(CC(=O)O)NC(=O)C(CC(C)C)n2cc(C3CCN(C(C)C)CC3)c(C(F)(F)F)cc2=O)c1F] |
| | 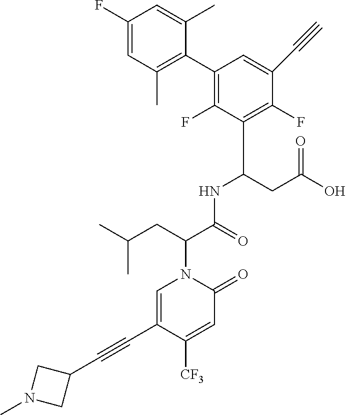 [SMILES: C#Cc1cc(-c2c(C)cc(F)cc2C)c(F)c(C(CC(=O)O)NC(=O)C(CC(C)C)n2cc(C#CC3CN(C)C3)c(C(F)(F)F)cc2=O)c1F] |
| | 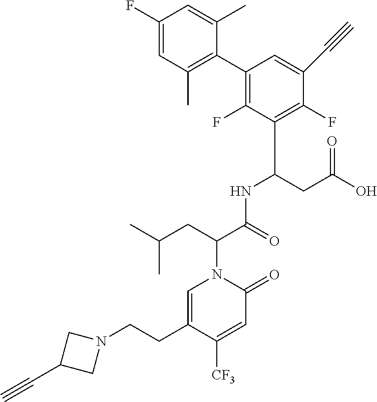 [SMILES: C#Cc1cc(-c2c(C)cc(F)cc2C)c(F)c(C(CC(=O)O)NC(=O)C(CC(C)C)n2cc(CCN3CC(C#C)C3)c(C(F)(F)F)cc2=O)c1F] |
| | 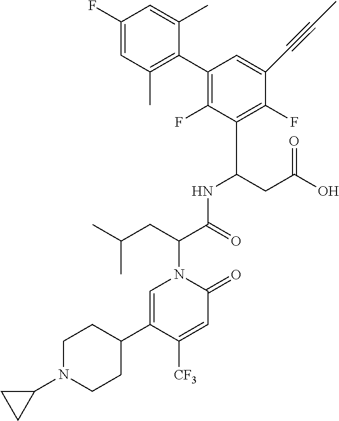 [SMILES: CC#Cc1cc(-c2c(C)cc(F)cc2C)c(F)c(C(CC(=O)O)NC(=O)C(CC(C)C)n2cc(C3CCN(C4CC4)CC3)c(C(F)(F)F)cc2=O)c1F] |
| 250 | 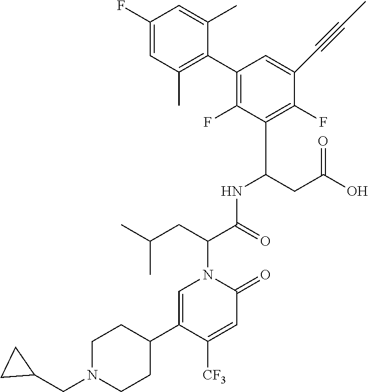 [SMILES: CC#Cc1cc(-c2c(C)cc(F)cc2C)c(F)c(C(CC(=O)O)NC(=O)C(CC(C)C)n2cc(C3CCN(CC4CC4)CC3)c(C(F)(F)F)cc2=O)c1F] |
| | 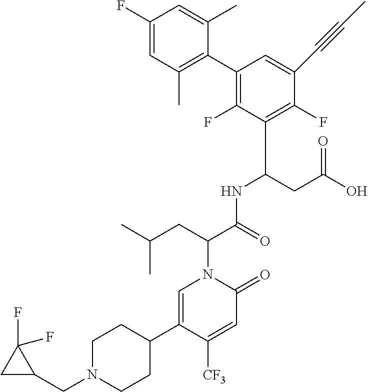 [SMILES: CC#Cc1cc(-c2c(C)cc(F)cc2C)c(F)c(C(CC(=O)O)NC(=O)C(CC(C)C)n2cc(C3CCN(CC4CC4(F)F)CC3)c(C(F)(F)F)cc2=O)c1F] |
| | 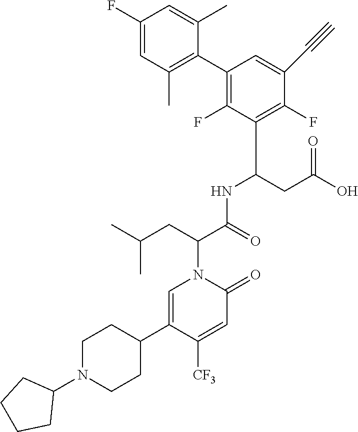 [SMILES: C#Cc1cc(-c2c(C)cc(F)cc2C)c(F)c(C(CC(=O)O)NC(=O)C(CC(C)C)n2cc(C3CCN(C4CCCC4)CC3)c(C(F)(F)F)cc2=O)c1F] |
| | 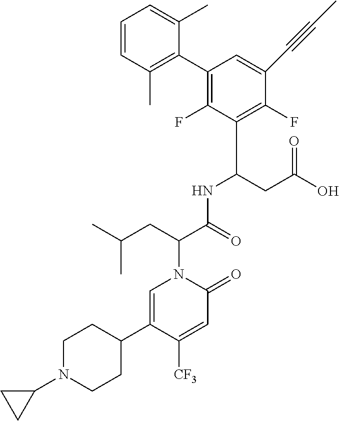 [SMILES: CC#Cc1cc(-c2c(C)cccc2C)c(F)c(C(CC(=O)O)NC(=O)C(CC(C)C)n2cc(C3CCN(C4CC4)CC3)c(C(F)(F)F)cc2=O)c1F] |
| | 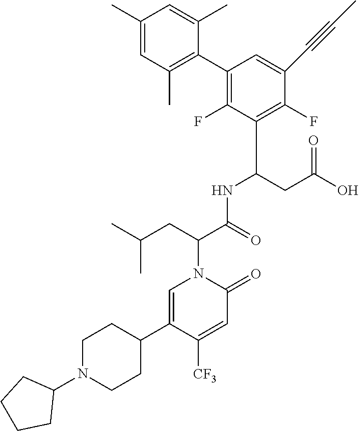 [SMILES: CC#Cc1cc(-c2c(C)cc(C)cc2C)c(F)c(C(CC(=O)O)NC(=O)C(CC(C)C)n2cc(C3CCN(C4CCCC4)CC3)c(C(F)(F)F)cc2=O)c1F] |
| 251 | 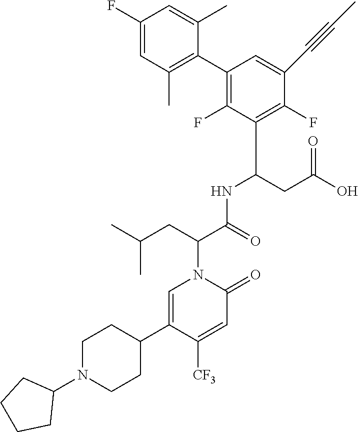 [SMILES: CC#Cc1cc(-c2c(C)cc(F)cc2C)c(F)c(C(CC(=O)O)NC(=O)C(CC(C)C)n2cc(C3CCN(C4CCCC4)CC3)c(C(F)(F)F)cc2=O)c1F] |
| | 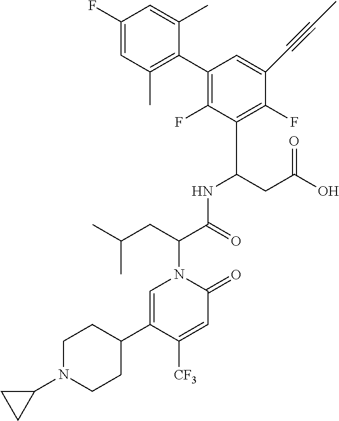 [SMILES: CC#Cc1cc(-c2c(C)cc(F)cc2C)c(F)c(C(CC(=O)O)NC(=O)C(CC(C)C)n2cc(C3CCN(C4CC4)CC3)c(C(F)(F)F)cc2=O)c1F] |
| | 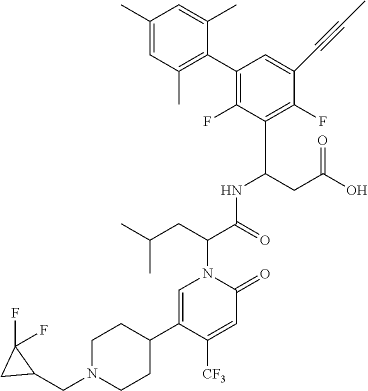 [SMILES: CC#Cc1cc(-c2c(C)cc(C)cc2C)c(F)c(C(CC(=O)O)NC(=O)C(CC(C)C)n2cc(C3CCN(CC4CC4(F)F)CC3)c(C(F)(F)F)cc2=O)c1F] |
| | 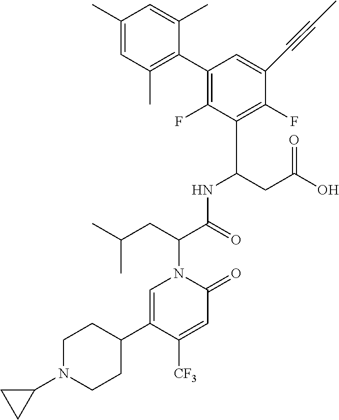 [SMILES: CC#Cc1cc(-c2c(C)cc(C)cc2C)c(F)c(C(CC(=O)O)NC(=O)C(CC(C)C)n2cc(C3CCN(C4CC4)CC3)c(C(F)(F)F)cc2=O)c1F] |
| | 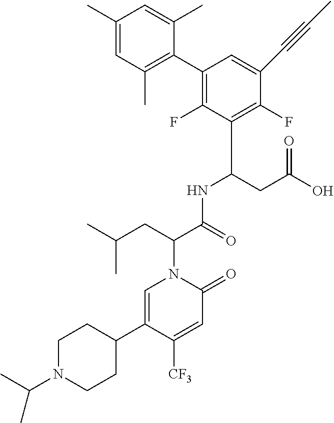 [SMILES: CC#Cc1cc(-c2c(C)cc(C)cc2C)c(F)c(C(CC(=O)O)NC(=O)C(CC(C)C)n2cc(C3CCN(C(C)C)CC3)c(C(F)(F)F)cc2=O)c1F] |
| 252 | 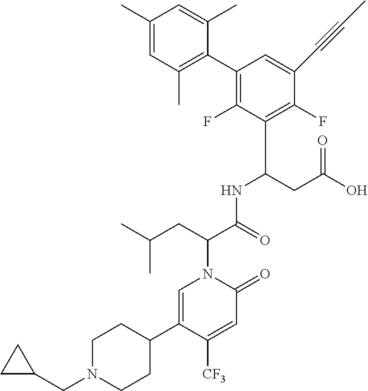 [SMILES: CC#Cc1cc(-c2c(C)cc(C)cc2C)c(F)c(C(CC(=O)O)NC(=O)C(CC(C)C)n2cc(C3CCN(CC4CC4)CC3)c(C(F)(F)F)cc2=O)c1F] |
| | 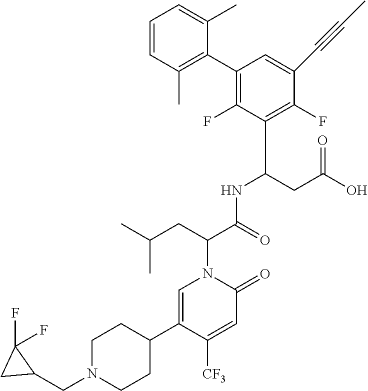 [SMILES: CC#Cc1cc(-c2c(C)cccc2C)c(F)c(C(CC(=O)O)NC(=O)C(CC(C)C)n2cc(C3CCN(CC4CC4(F)F)CC3)c(C(F)(F)F)cc2=O)c1F] |
| | 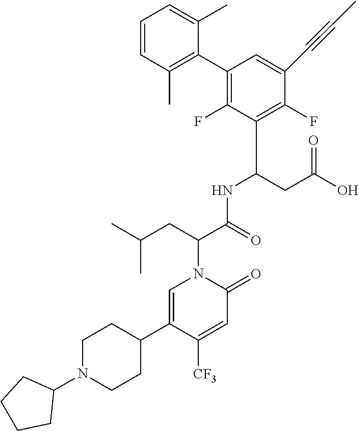 [SMILES: CC#Cc1cc(-c2c(C)cccc2C)c(F)c(C(CC(=O)O)NC(=O)C(CC(C)C)n2cc(C3CCN(C4CCCC4)CC3)c(C(F)(F)F)cc2=O)c1F] |
| | 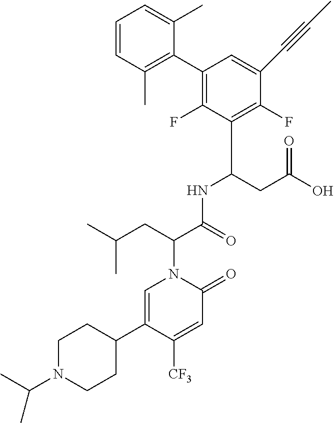 [SMILES: CC#Cc1cc(-c2c(C)cccc2C)c(F)c(C(CC(=O)O)NC(=O)C(CC(C)C)n2cc(C3CCN(C(C)C)CC3)c(C(F)(F)F)cc2=O)c1F] |
| | 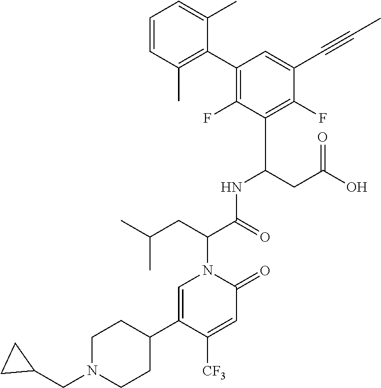 [SMILES: CC#Cc1cc(-c2c(C)cccc2C)c(F)c(C(CC(=O)O)NC(=O)C(CC(C)C)n2cc(C3CCN(CC4CC4)CC3)c(C(F)(F)F)cc2=O)c1F] |
| 253 | 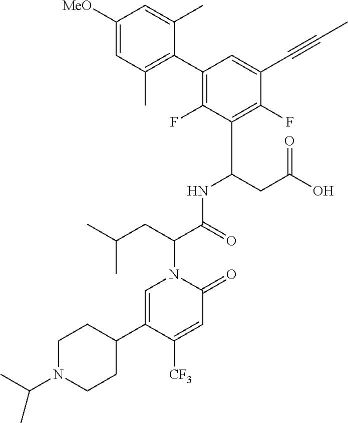 [SMILES: CC#Cc1cc(-c2c(C)cc(OC)cc2C)c(F)c(C(CC(=O)O)NC(=O)C(CC(C)C)n2cc(C3CCN(C(C)C)CC3)c(C(F)(F)F)cc2=O)c1F] |
| | 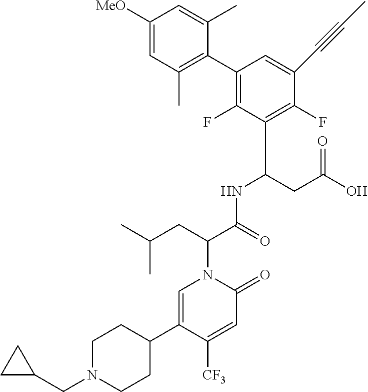 [SMILES: CC#Cc1cc(-c2c(C)cc(OC)cc2C)c(F)c(C(CC(=O)O)NC(=O)C(CC(C)C)n2cc(C3CCN(CC4CC4)CC3)c(C(F)(F)F)cc2=O)c1F] |
| | 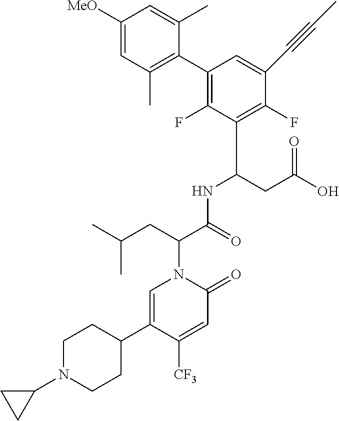 [SMILES: CC#Cc1cc(-c2c(C)cc(OC)cc2C)c(F)c(C(CC(=O)O)NC(=O)C(CC(C)C)n2cc(C3CCN(C4CC4)CC3)c(C(F)(F)F)cc2=O)c1F] |
| | 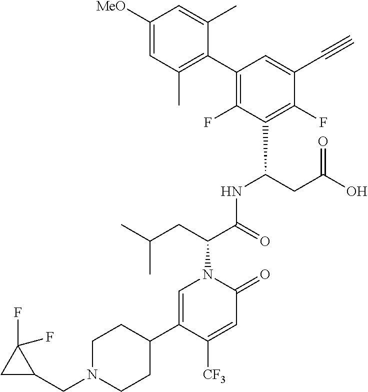 [SMILES: C#Cc1cc(-c2c(C)cc(OC)cc2C)c(F)c([C@H](CC(=O)O)NC(=O)[C@@H](CC(C)C)n2cc(C3CCN(CC4CC4(F)F)CC3)c(C(F)(F)F)cc2=O)c1F] |
| | 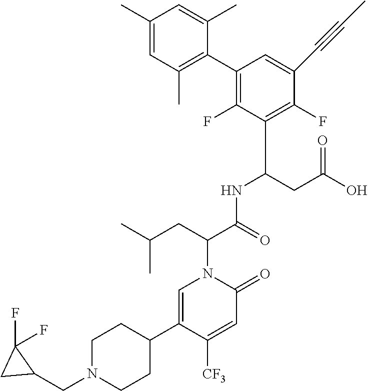 [SMILES: CC#Cc1cc(-c2c(C)cc(C)cc2C)c(F)c(C(CC(=O)O)NC(=O)C(CC(C)C)n2cc(C3CCN(CC4CC4(F)F)CC3)c(C(F)(F)F)cc2=O)c1F] |
| 254 | 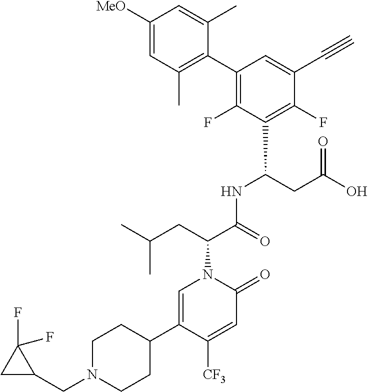 [SMILES: C#Cc1cc(-c2c(C)cc(OC)cc2C)c(F)c([C@H](CC(=O)O)NC(=O)[C@@H](CC(C)C)n2cc(C3CCN(CC4CC4(F)F)CC3)c(C(F)(F)F)cc2=O)c1F] |
| | 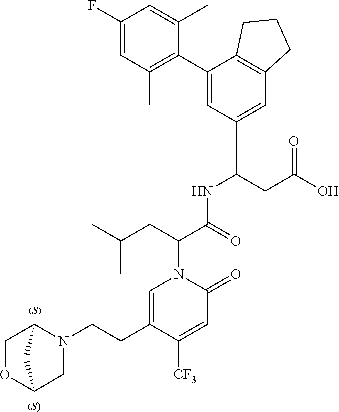 |
| | 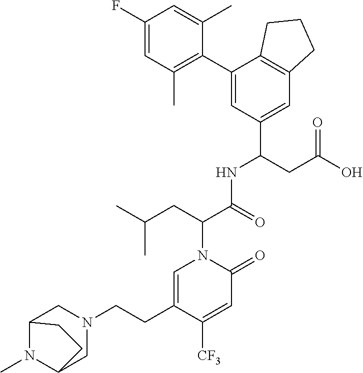 [SMILES: Cc1cc(F)cc(C)c1-c1cc(C(CC(=O)O)NC(=O)C(CC(C)C)n2cc(CCN3CC4CCC(C3)N4C)c(C(F)(F)F)cc2=O)cc2c1CCC2] |
| | 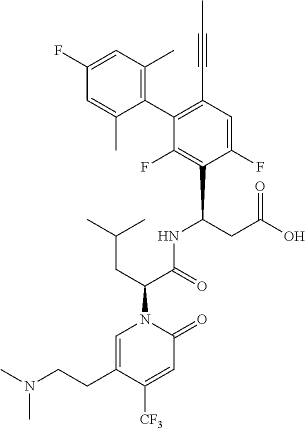 [SMILES: CC#Cc1cc(F)c([C@@H](CC(=O)O)NC(=O)[C@H](CC(C)C)n2cc(CCN(C)C)c(C(F)(F)F)cc2=O)c(F)c1-c1c(C)cc(F)cc1C] |
| | 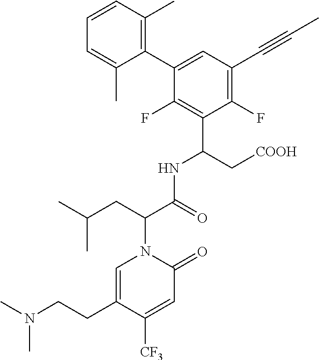 [SMILES: CC#Cc1cc(-c2c(C)cccc2C)c(F)c(C(CC(=O)O)NC(=O)C(CC(C)C)n2cc(CCN(C)C)c(C(F)(F)F)cc2=O)c1F] |
| 255 | 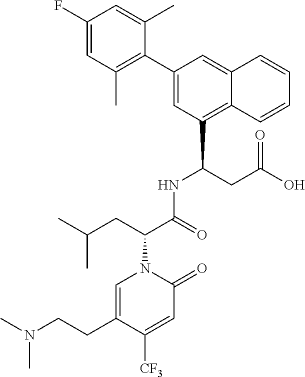 [SMILES: Cc1cc(F)cc(C)c1-c1cc([C@@H](CC(=O)O)NC(=O)[C@@H](CC(C)C)n2cc(CCN(C)C)c(C(F)(F)F)cc2=O)c2ccccc2c1] |
| | 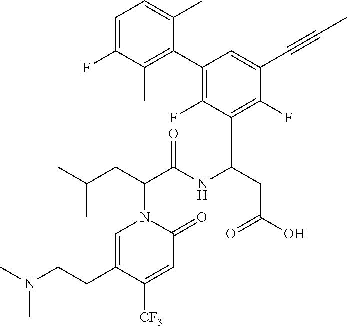 [SMILES: CC#Cc1cc(-c2c(C)ccc(F)c2C)c(F)c(C(CC(=O)O)NC(=O)C(CC(C)C)n2cc(CCN(C)C)c(C(F)(F)F)cc2=O)c1F] |
| | 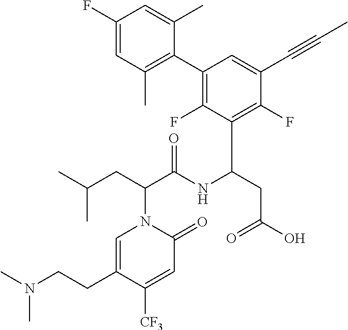 [SMILES: CC#Cc1cc(-c2c(C)cc(F)cc2C)c(F)c(C(CC(=O)O)NC(=O)C(CC(C)C)n2cc(CCN(C)C)c(C(F)(F)F)cc2=O)c1F] |
| | 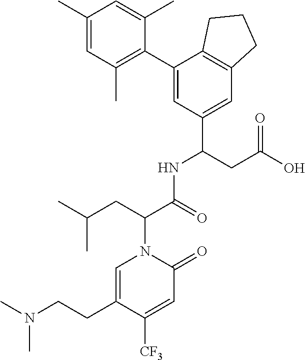 [SMILES: Cc1cc(C)c(-c2cc(C(CC(=O)O)NC(=O)C(CC(C)C)n3cc(CCN(C)C)c(C(F)(F)F)cc3=O)cc3c2CCC3)c(C)c1] |
| | 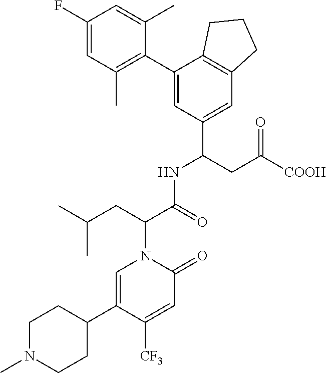 [SMILES: Cc1cc(F)cc(C)c1-c1cc(C(CC(=O)C(=O)O)NC(=O)C(CC(C)C)n2cc(C3CCN(C)CC3)c(C(F)(F)F)cc2=O)cc2c1CCC2] |

TABLE E-3

| | |
|---|---|
| 256 | 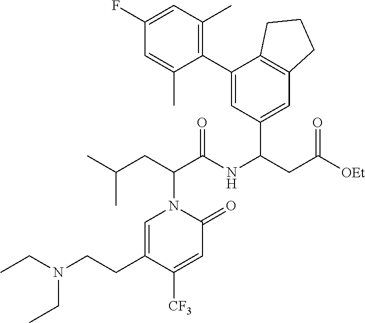 [SMILES: CCOC(=O)CC(NC(=O)C(CC(C)C)n1cc(CCN(CC)CC)c(C(F)(F)F)cc1=O)c1cc2c(c(-c3c(C)cc(F)cc3C)c1)CCC2] |
| | 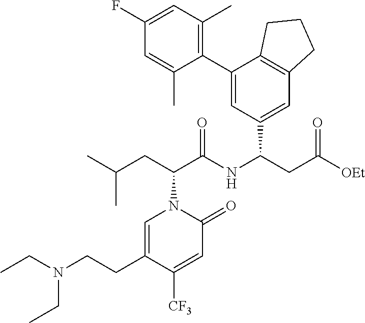 [SMILES: CCOC(=O)C[C@H](NC(=O)[C@@H](CC(C)C)n1cc(CCN(CC)CC)c(C(F)(F)F)cc1=O)c1cc2c(c(-c3c(C)cc(F)cc3C)c1)CCC2] |
| | 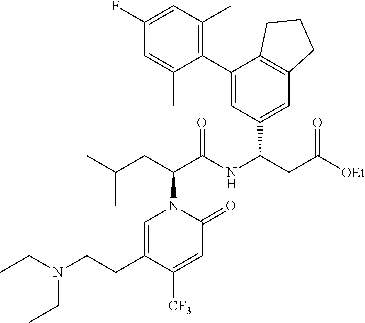 [SMILES: CCOC(=O)C[C@H](NC(=O)[C@H](CC(C)C)n1cc(CCN(CC)CC)c(C(F)(F)F)cc1=O)c1cc2c(c(-c3c(C)cc(F)cc3C)c1)CCC2] |
| | 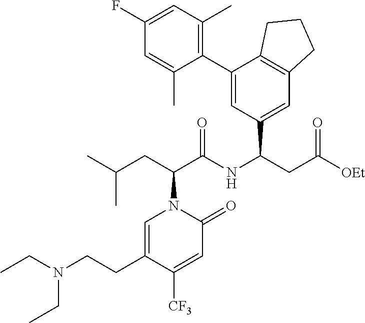 [SMILES: CCOC(=O)C[C@@H](NC(=O)[C@H](CC(C)C)n1cc(CCN(CC)CC)c(C(F)(F)F)cc1=O)c1cc2c(c(-c3c(C)cc(F)cc3C)c1)CCC2] |
| | 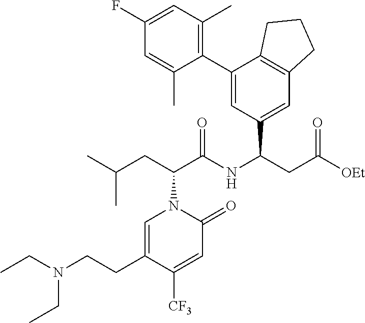 [SMILES: CCOC(=O)C[C@@H](NC(=O)[C@@H](CC(C)C)n1cc(CCN(CC)CC)c(C(F)(F)F)cc1=O)c1cc2c(c(-c3c(C)cc(F)cc3C)c1)CCC2] |
| 257 | 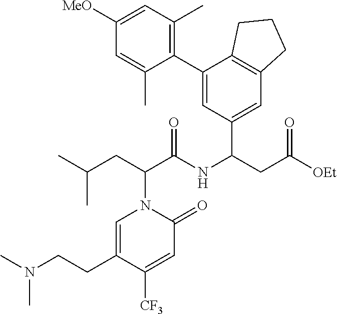 [SMILES: CCOC(=O)CC(NC(=O)C(CC(C)C)n1cc(CCN(C)C)c(C(F)(F)F)cc1=O)c1cc2c(c(-c3c(C)cc(OC)cc3C)c1)CCC2] |
| | 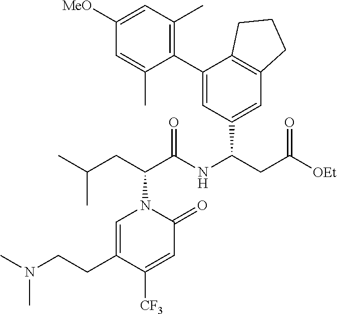 [SMILES: CCOC(=O)C[C@H](NC(=O)[C@@H](CC(C)C)n1cc(CCN(C)C)c(C(F)(F)F)cc1=O)c1cc2c(c(-c3c(C)cc(OC)cc3C)c1)CCC2] |
| | 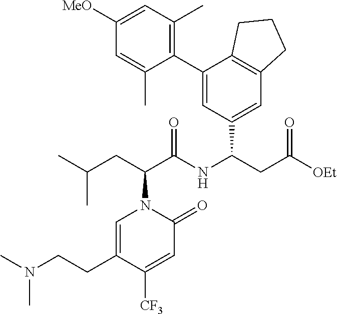 [SMILES: CCOC(=O)C[C@H](NC(=O)[C@H](CC(C)C)n1cc(CCN(C)C)c(C(F)(F)F)cc1=O)c1cc2c(c(-c3c(C)cc(OC)cc3C)c1)CCC2] |
| | 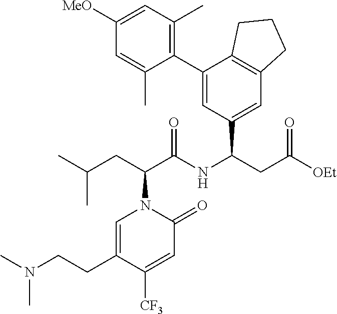 [SMILES: CCOC(=O)C[C@@H](NC(=O)[C@H](CC(C)C)n1cc(CCN(C)C)c(C(F)(F)F)cc1=O)c1cc2c(c(-c3c(C)cc(OC)cc3C)c1)CCC2] |
| | 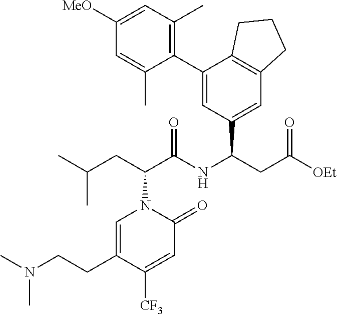 [SMILES: CCOC(=O)C[C@@H](NC(=O)[C@@H](CC(C)C)n1cc(CCN(C)C)c(C(F)(F)F)cc1=O)c1cc2c(c(-c3c(C)cc(OC)cc3C)c1)CCC2] |
| 258 | 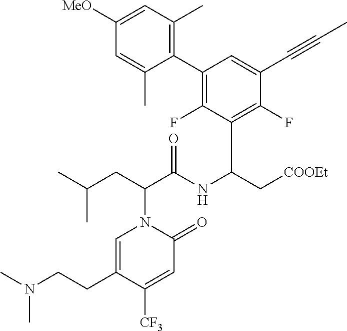 [SMILES: CC#Cc1cc(-c2c(C)cc(OC)cc2C)c(F)c(C(CC(=O)OCC)NC(=O)C(CC(C)C)n2cc(CCN(C)C)c(C(F)(F)F)cc2=O)c1F] |
| | 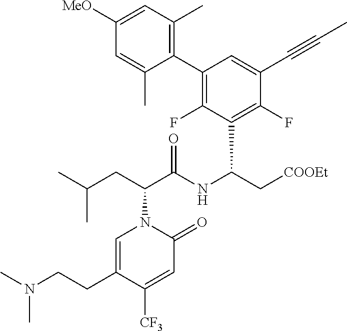 [SMILES: CC#Cc1cc(-c2c(C)cc(OC)cc2C)c(F)c([C@H](CC(=O)OCC)NC(=O)[C@@H](CC(C)C)n2cc(CCN(C)C)c(C(F)(F)F)cc2=O)c1F] |
| | 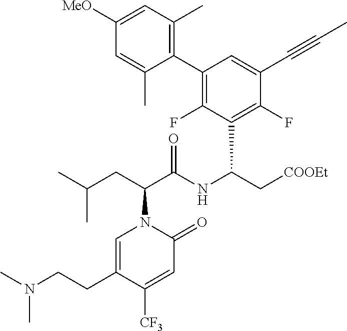 [SMILES: CC#Cc1cc(-c2c(C)cc(OC)cc2C)c(F)c([C@H](CC(=O)OCC)NC(=O)[C@H](CC(C)C)n2cc(CCN(C)C)c(C(F)(F)F)cc2=O)c1F] |
| | 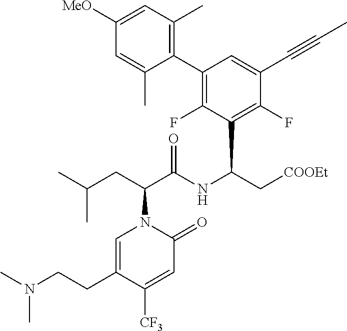 [SMILES: CC#Cc1cc(-c2c(C)cc(OC)cc2C)c(F)c([C@@H](CC(=O)OCC)NC(=O)[C@H](CC(C)C)n2cc(CCN(C)C)c(C(F)(F)F)cc2=O)c1F] |
| | 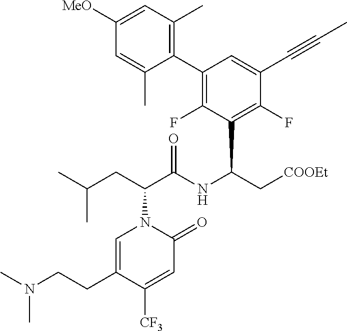 [SMILES: CC#Cc1cc(-c2c(C)cc(OC)cc2C)c(F)c([C@@H](CC(=O)OCC)NC(=O)[C@@H](CC(C)C)n2cc(CCN(C)C)c(C(F)(F)F)cc2=O)c1F] |
| 259 | 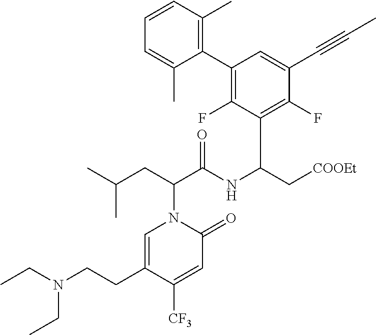 [SMILES: CC#Cc1cc(-c2c(C)cccc2C)c(F)c(C(CC(=O)OCC)NC(=O)C(CC(C)C)n2cc(CCN(CC)CC)c(C(F)(F)F)cc2=O)c1F] |
| | 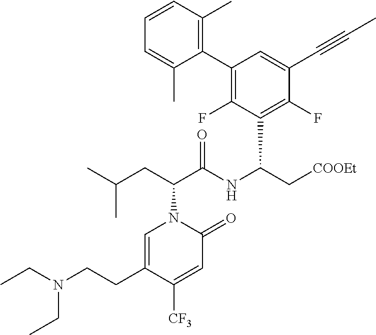 [SMILES: CC#Cc1cc(-c2c(C)cccc2C)c(F)c([C@H](CC(=O)OCC)NC(=O)[C@@H](CC(C)C)n2cc(CCN(CC)CC)c(C(F)(F)F)cc2=O)c1F] |
| | 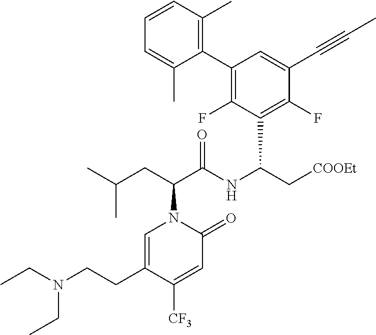 [SMILES: CC#Cc1cc(-c2c(C)cccc2C)c(F)c([C@H](CC(=O)OCC)NC(=O)[C@H](CC(C)C)n2cc(CCN(CC)CC)c(C(F)(F)F)cc2=O)c1F] |
| | 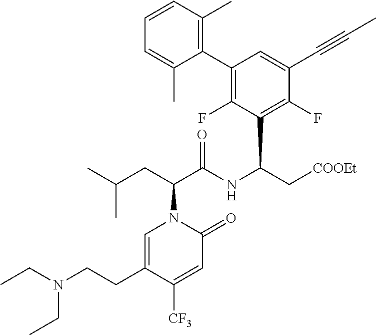 [SMILES: CC#Cc1cc(-c2c(C)cccc2C)c(F)c([C@@H](CC(=O)OCC)NC(=O)[C@H](CC(C)C)n2cc(CCN(CC)CC)c(C(F)(F)F)cc2=O)c1F] |
| | 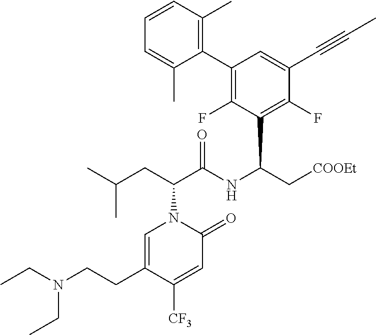 [SMILES: CC#Cc1cc(-c2c(C)cccc2C)c(F)c([C@@H](CC(=O)OCC)NC(=O)[C@@H](CC(C)C)n2cc(CCN(CC)CC)c(C(F)(F)F)cc2=O)c1F] |
| 260 | 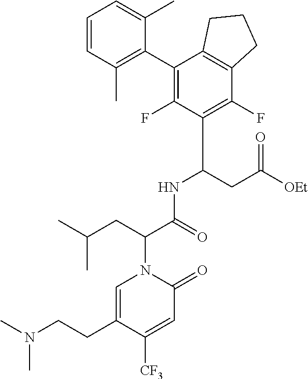 [SMILES: CCOC(=O)CC(NC(=O)C(CC(C)C)n1cc(CCN(C)C)c(C(F)(F)F)cc1=O)c1c(F)c2c(c(-c3c(C)cccc3C)c1F)CCC2] |
| | 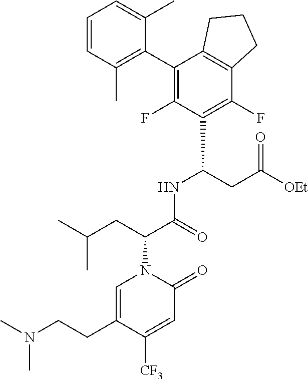 [SMILES: CCOC(=O)C[C@H](NC(=O)[C@@H](CC(C)C)n1cc(CCN(C)C)c(C(F)(F)F)cc1=O)c1c(F)c2c(c(-c3c(C)cccc3C)c1F)CCC2] |
| | 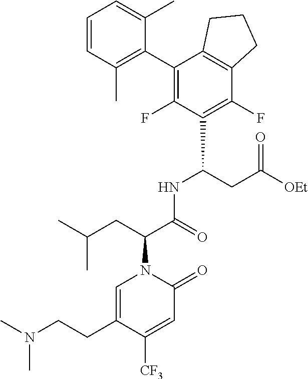 [SMILES: CCOC(=O)C[C@H](NC(=O)[C@H](CC(C)C)n1cc(CCN(C)C)c(C(F)(F)F)cc1=O)c1c(F)c2c(c(-c3c(C)cccc3C)c1F)CCC2] |
| | 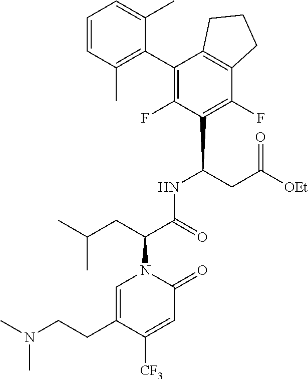 [SMILES: CCOC(=O)C[C@@H](NC(=O)[C@H](CC(C)C)n1cc(CCN(C)C)c(C(F)(F)F)cc1=O)c1c(F)c2c(c(-c3c(C)cccc3C)c1F)CCC2] |
| | 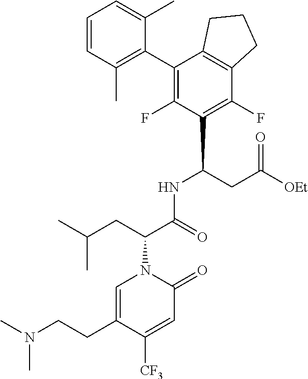 [SMILES: CCOC(=O)C[C@@H](NC(=O)[C@@H](CC(C)C)n1cc(CCN(C)C)c(C(F)(F)F)cc1=O)c1c(F)c2c(c(-c3c(C)cccc3C)c1F)CCC2] |
| 261 | 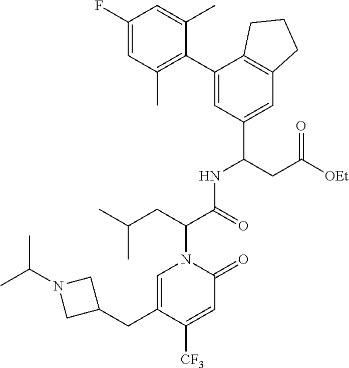 [SMILES: CCOC(=O)CC(NC(=O)C(CC(C)C)n1cc(CC2CN(C(C)C)C2)c(C(F)(F)F)cc1=O)c1cc2c(c(-c3c(C)cc(F)cc3C)c1)CCC2] |
| | 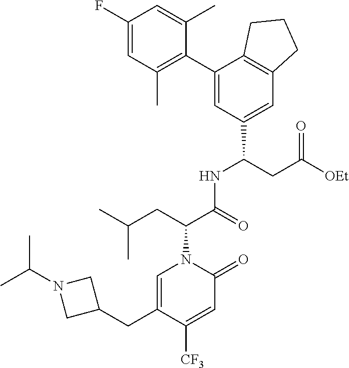 [SMILES: CCOC(=O)C[C@H](NC(=O)[C@@H](CC(C)C)n1cc(CC2CN(C(C)C)C2)c(C(F)(F)F)cc1=O)c1cc2c(c(-c3c(C)cc(F)cc3C)c1)CCC2] |
| | 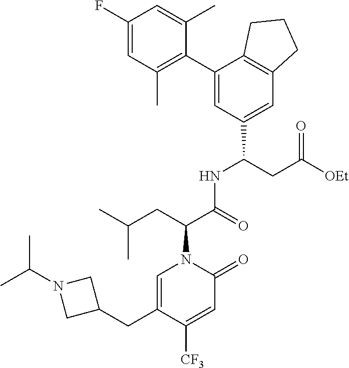 [SMILES: CCOC(=O)C[C@H](NC(=O)[C@H](CC(C)C)n1cc(CC2CN(C(C)C)C2)c(C(F)(F)F)cc1=O)c1cc2c(c(-c3c(C)cc(F)cc3C)c1)CCC2] |
| | 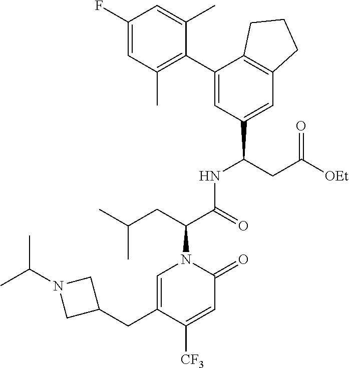 [SMILES: CCOC(=O)C[C@@H](NC(=O)[C@H](CC(C)C)n1cc(CC2CN(C(C)C)C2)c(C(F)(F)F)cc1=O)c1cc2c(c(-c3c(C)cc(F)cc3C)c1)CCC2] |
| | 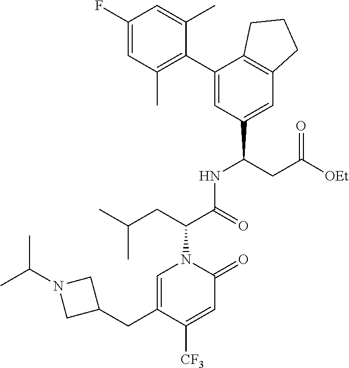 [SMILES: CCOC(=O)C[C@@H](NC(=O)[C@@H](CC(C)C)n1cc(CC2CN(C(C)C)C2)c(C(F)(F)F)cc1=O)c1cc2c(c(-c3c(C)cc(F)cc3C)c1)CCC2] |
| 262 | 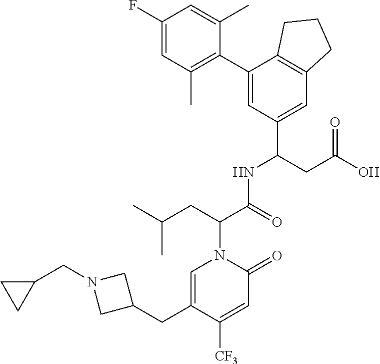 [SMILES: Cc1cc(F)cc(C)c1-c1cc(C(CC(=O)O)NC(=O)C(CC(C)C)n2cc(CC3CN(CC4CC4)C3)c(C(F)(F)F)cc2=O)cc2c1CCC2] |
| | 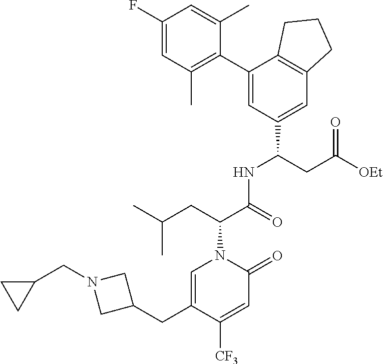 [SMILES: CCOC(=O)C[C@H](NC(=O)[C@@H](CC(C)C)n1cc(CC2CN(CC3CC3)C2)c(C(F)(F)F)cc1=O)c1cc2c(c(-c3c(C)cc(F)cc3C)c1)CCC2] |
| | 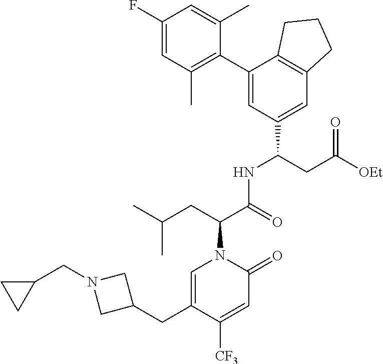 [SMILES: CCOC(=O)C[C@H](NC(=O)[C@H](CC(C)C)n1cc(CC2CN(CC3CC3)C2)c(C(F)(F)F)cc1=O)c1cc2c(c(-c3c(C)cc(F)cc3C)c1)CCC2] |
| | 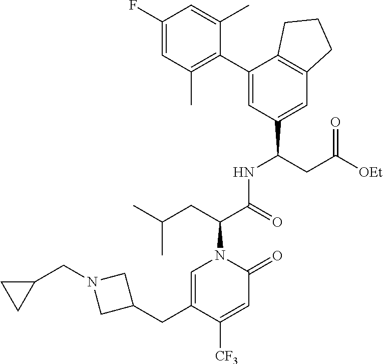 [SMILES: CCOC(=O)C[C@@H](NC(=O)[C@H](CC(C)C)n1cc(CC2CN(CC3CC3)C2)c(C(F)(F)F)cc1=O)c1cc2c(c(-c3c(C)cc(F)cc3C)c1)CCC2] |
| | 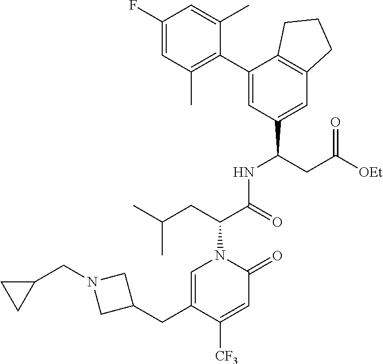 [SMILES: CCOC(=O)C[C@@H](NC(=O)[C@@H](CC(C)C)n1cc(CC2CN(CC3CC3)C2)c(C(F)(F)F)cc1=O)c1cc2c(c(-c3c(C)cc(F)cc3C)c1)CCC2] |
| 263 | 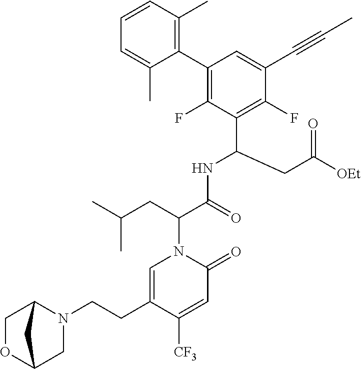 [SMILES: CC#Cc1cc(-c2c(C)cccc2C)c(F)c(C(CC(=O)OCC)NC(=O)C(CC(C)C)n2cc(CCN3C[C@H]4C[C@@H]3CO4)c(C(F)(F)F)cc2=O)c1F] |
| | 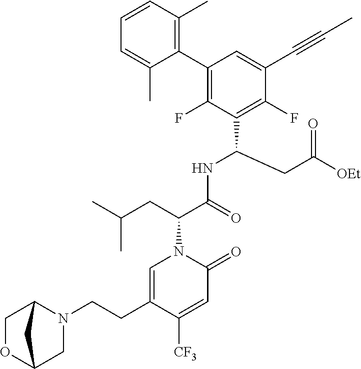 [SMILES: CC#Cc1cc(-c2c(C)cccc2C)c(F)c([C@H](CC(=O)OCC)NC(=O)[C@@H](CC(C)C)n2cc(CCN3C[C@H]4C[C@@H]3CO4)c(C(F)(F)F)cc2=O)c1F] |
| | 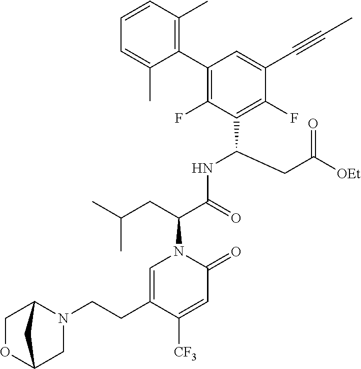 [SMILES: CC#Cc1cc(-c2c(C)cccc2C)c(F)c([C@H](CC(=O)OCC)NC(=O)[C@H](CC(C)C)n2cc(CCN3C[C@H]4C[C@@H]3CO4)c(C(F)(F)F)cc2=O)c1F] |
| | 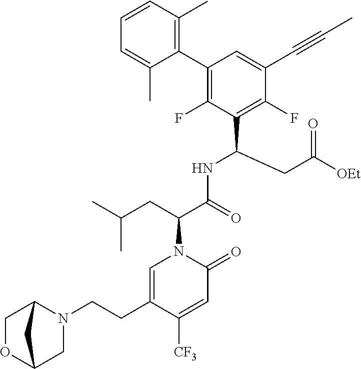 [SMILES: CC#Cc1cc(-c2c(C)cccc2C)c(F)c([C@@H](CC(=O)OCC)NC(=O)[C@H](CC(C)C)n2cc(CCN3C[C@H]4C[C@@H]3CO4)c(C(F)(F)F)cc2=O)c1F] |
| | 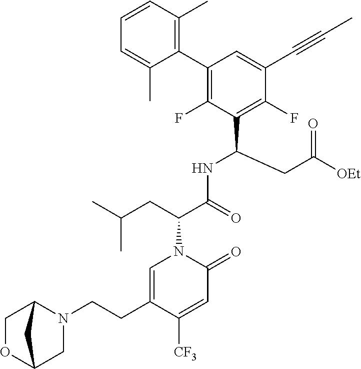 [SMILES: CC#Cc1cc(-c2c(C)cccc2C)c(F)c([C@@H](CC(=O)OCC)NC(=O)[C@@H](CC(C)C)n2cc(CCN3C[C@H]4C[C@@H]3CO4)c(C(F)(F)F)cc2=O)c1F] |
| 264 | 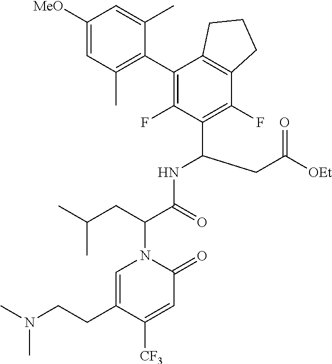 [SMILES: CCOC(=O)CC(NC(=O)C(CC(C)C)n1cc(CCN(C)C)c(C(F)(F)F)cc1=O)c1c(F)c2c(c(-c3c(C)cc(OC)cc3C)c1F)CCC2] |
| | 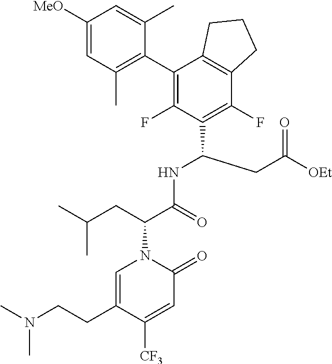 [SMILES: CCOC(=O)C[C@H](NC(=O)[C@@H](CC(C)C)n1cc(CCN(C)C)c(C(F)(F)F)cc1=O)c1c(F)c2c(c(-c3c(C)cc(OC)cc3C)c1F)CCC2] |
| | 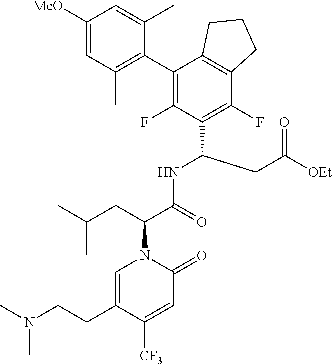 [SMILES: CCOC(=O)C[C@H](NC(=O)[C@H](CC(C)C)n1cc(CCN(C)C)c(C(F)(F)F)cc1=O)c1c(F)c2c(c(-c3c(C)cc(OC)cc3C)c1F)CCC2] |
| | 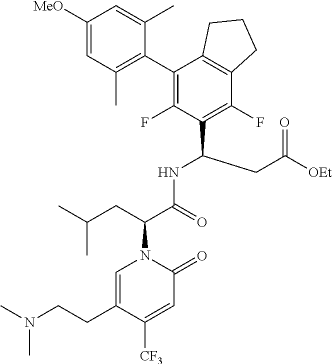 [SMILES: CCOC(=O)C[C@@H](NC(=O)[C@H](CC(C)C)n1cc(CCN(C)C)c(C(F)(F)F)cc1=O)c1c(F)c2c(c(-c3c(C)cc(OC)cc3C)c1F)CCC2] |
| | 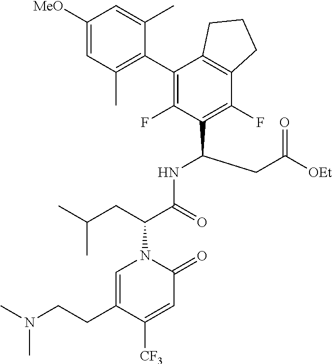 [SMILES: CCOC(=O)C[C@@H](NC(=O)[C@@H](CC(C)C)n1cc(CCN(C)C)c(C(F)(F)F)cc1=O)c1c(F)c2c(c(-c3c(C)cc(OC)cc3C)c1F)CCC2] |
| 265 | 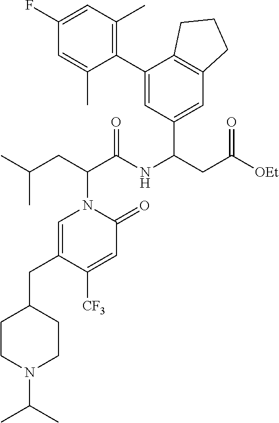 [SMILES: CCOC(=O)CC(NC(=O)C(CC(C)C)n1cc(CC2CCN(C(C)C)CC2)c(C(F)(F)F)cc1=O)c1cc2c(c(-c3c(C)cc(F)cc3C)c1)CCC2] |
| | 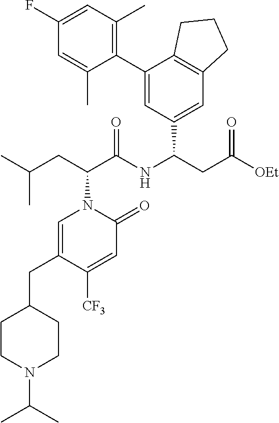 [SMILES: CCOC(=O)C[C@H](NC(=O)[C@@H](CC(C)C)n1cc(CC2CCN(C(C)C)CC2)c(C(F)(F)F)cc1=O)c1cc2c(c(-c3c(C)cc(F)cc3C)c1)CCC2] |
| | 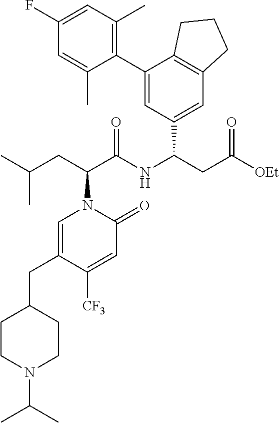 [SMILES: CCOC(=O)C[C@H](NC(=O)[C@H](CC(C)C)n1cc(CC2CCN(C(C)C)CC2)c(C(F)(F)F)cc1=O)c1cc2c(c(-c3c(C)cc(F)cc3C)c1)CCC2] |
| | 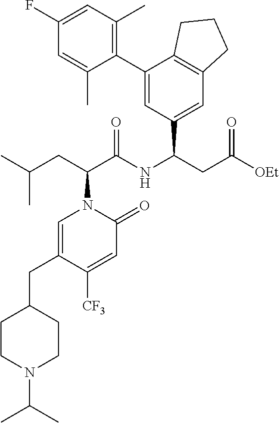 [SMILES: CCOC(=O)C[C@@H](NC(=O)[C@H](CC(C)C)n1cc(CC2CCN(C(C)C)CC2)c(C(F)(F)F)cc1=O)c1cc2c(c(-c3c(C)cc(F)cc3C)c1)CCC2] |
| | 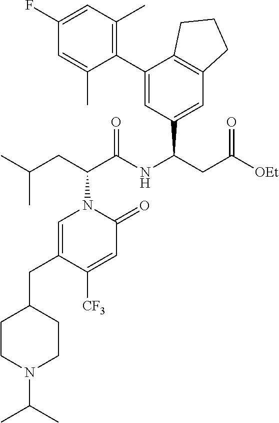 [SMILES: CCOC(=O)C[C@@H](NC(=O)[C@@H](CC(C)C)n1cc(CC2CCN(C(C)C)CC2)c(C(F)(F)F)cc1=O)c1cc2c(c(-c3c(C)cc(F)cc3C)c1)CCC2] |
| 266 | 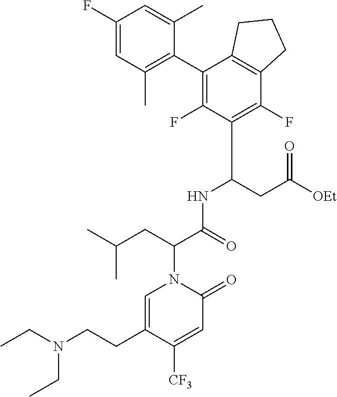 [SMILES: CCOC(=O)CC(NC(=O)C(CC(C)C)n1cc(CCN(CC)CC)c(C(F)(F)F)cc1=O)c1c(F)c2c(c(-c3c(C)cc(F)cc3C)c1F)CCC2] |
| | 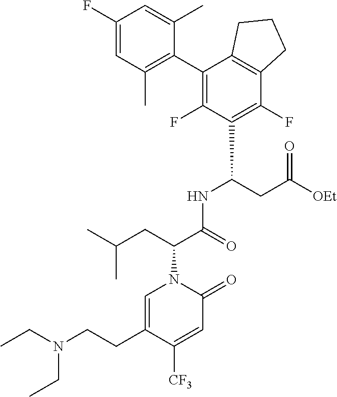 [SMILES: CCOC(=O)C[C@H](NC(=O)[C@@H](CC(C)C)n1cc(CCN(CC)CC)c(C(F)(F)F)cc1=O)c1c(F)c2c(c(-c3c(C)cc(F)cc3C)c1F)CCC2] |
| | 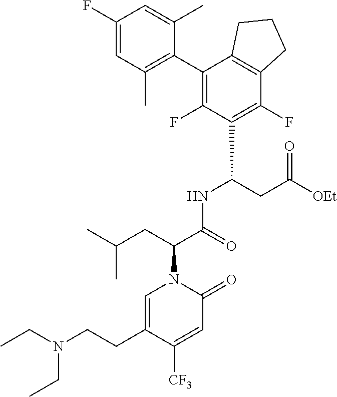 [SMILES: CCOC(=O)C[C@H](NC(=O)[C@H](CC(C)C)n1cc(CCN(CC)CC)c(C(F)(F)F)cc1=O)c1c(F)c2c(c(-c3c(C)cc(F)cc3C)c1F)CCC2] |
| | 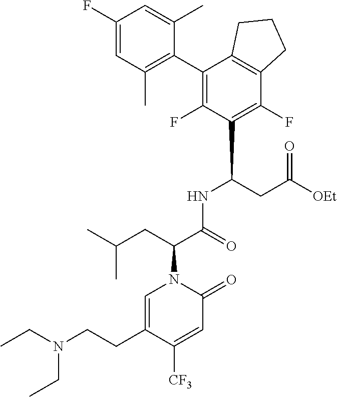 [SMILES: CCOC(=O)C[C@@H](NC(=O)[C@H](CC(C)C)n1cc(CCN(CC)CC)c(C(F)(F)F)cc1=O)c1c(F)c2c(c(-c3c(C)cc(F)cc3C)c1F)CCC2] |
| | 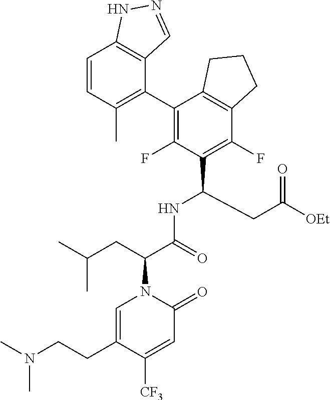 [SMILES: CCOC(=O)C[C@@H](NC(=O)[C@H](CC(C)C)n1cc(CCN(C)C)c(C(F)(F)F)cc1=O)c1c(F)c2c(c(-c3c(C)ccc4[nH]ncc34)c1F)CCC2] |
| 267 | 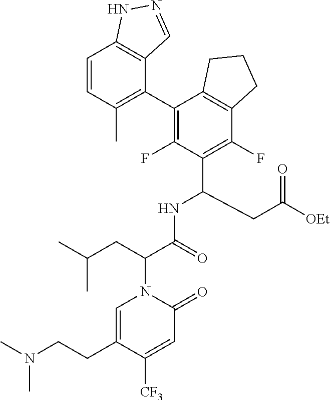 [SMILES: CCOC(=O)CC(NC(=O)C(CC(C)C)n1cc(CCN(C)C)c(C(F)(F)F)cc1=O)c1c(F)c2c(c(-c3c(C)ccc4[nH]ncc34)c1F)CCC2] |
| | 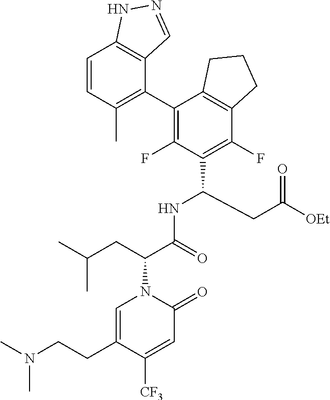 [SMILES: CCOC(=O)C[C@H](NC(=O)[C@@H](CC(C)C)n1cc(CCN(C)C)c(C(F)(F)F)cc1=O)c1c(F)c2c(c(-c3c(C)ccc4[nH]ncc34)c1F)CCC2] |
| | 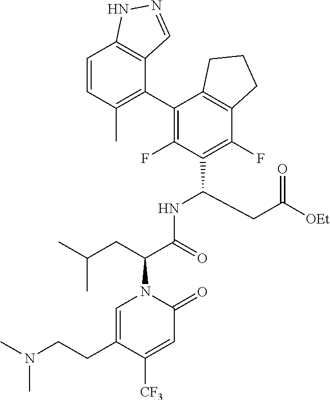 [SMILES: CCOC(=O)C[C@H](NC(=O)[C@H](CC(C)C)n1cc(CCN(C)C)c(C(F)(F)F)cc1=O)c1c(F)c2c(c(-c3c(C)ccc4[nH]ncc34)c1F)CCC2] |
| | 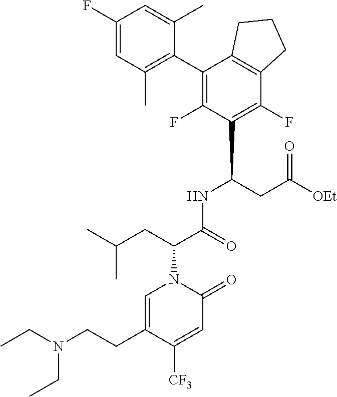 [SMILES: CCOC(=O)C[C@@H](NC(=O)[C@@H](CC(C)C)n1cc(CCN(CC)CC)c(C(F)(F)F)cc1=O)c1c(F)c2c(c(-c3c(C)cc(F)cc3C)c1F)CCC2] |
| | 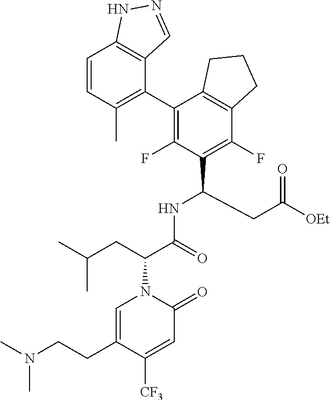 [SMILES: CCOC(=O)C[C@@H](NC(=O)[C@@H](CC(C)C)n1cc(CCN(C)C)c(C(F)(F)F)cc1=O)c1c(F)c2c(c(-c3c(C)ccc4[nH]ncc34)c1F)CCC2] |
| 268 | 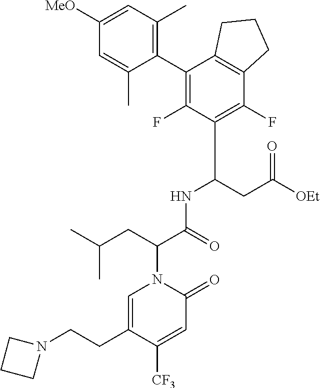 [SMILES: CCOC(=O)CC(NC(=O)C(CC(C)C)n1cc(CCN2CCC2)c(C(F)(F)F)cc1=O)c1c(F)c2c(c(-c3c(C)cc(OC)cc3C)c1F)CCC2] |
| | 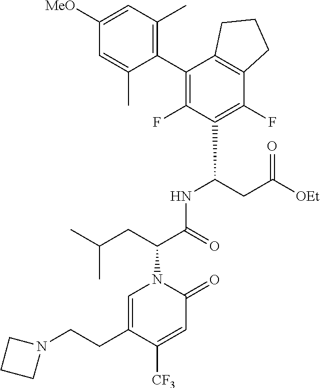 [SMILES: CCOC(=O)C[C@H](NC(=O)[C@@H](CC(C)C)n1cc(CCN2CCC2)c(C(F)(F)F)cc1=O)c1c(F)c2c(c(-c3c(C)cc(OC)cc3C)c1F)CCC2] |
| | 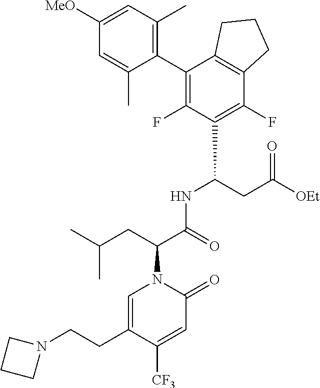 [SMILES: CCOC(=O)C[C@H](NC(=O)[C@H](CC(C)C)n1cc(CCN2CCC2)c(C(F)(F)F)cc1=O)c1c(F)c2c(c(-c3c(C)cc(OC)cc3C)c1F)CCC2] |
| | 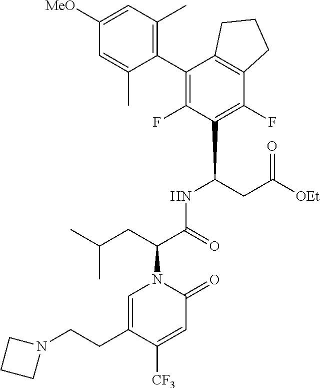 [SMILES: CCOC(=O)C[C@@H](NC(=O)[C@H](CC(C)C)n1cc(CCN2CCC2)c(C(F)(F)F)cc1=O)c1c(F)c2c(c(-c3c(C)cc(OC)cc3C)c1F)CCC2] |
| | 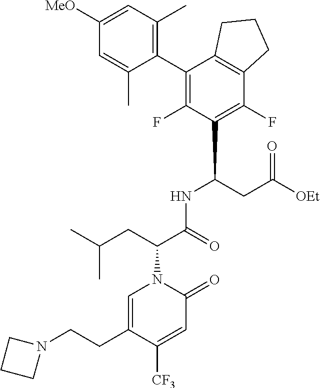 [SMILES: CCOC(=O)C[C@@H](NC(=O)[C@@H](CC(C)C)n1cc(CCN2CCC2)c(C(F)(F)F)cc1=O)c1c(F)c2c(c(-c3c(C)cc(OC)cc3C)c1F)CCC2] |
| 269 | 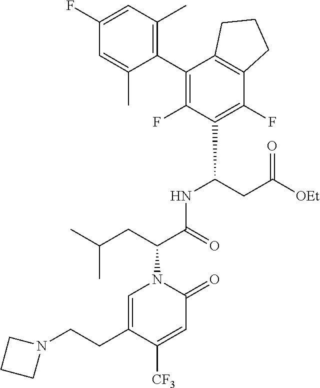 [SMILES: CCOC(=O)C[C@H](NC(=O)[C@@H](CC(C)C)n1cc(CCN2CCC2)c(C(F)(F)F)cc1=O)c1c(F)c2c(c(-c3c(C)cc(F)cc3C)c1F)CCC2] |
| | 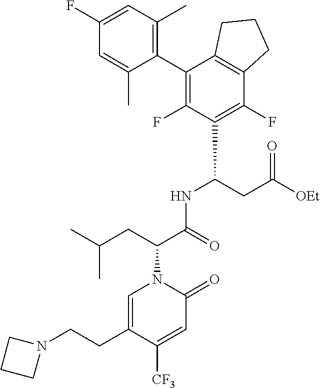 [SMILES: CCOC(=O)C[C@H](NC(=O)[C@@H](CC(C)C)n1cc(CCN2CCC2)c(C(F)(F)F)cc1=O)c1c(F)c2c(c(-c3c(C)cc(F)cc3C)c1F)CCC2] |
| | 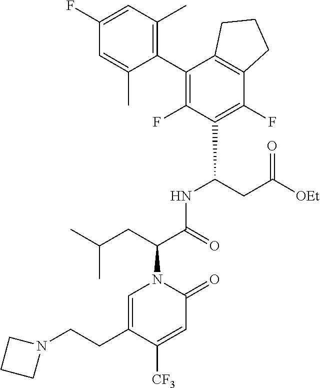 [SMILES: CCOC(=O)C[C@H](NC(=O)[C@H](CC(C)C)n1cc(CCN2CCC2)c(C(F)(F)F)cc1=O)c1c(F)c2c(c(-c3c(C)cc(F)cc3C)c1F)CCC2] |
| | 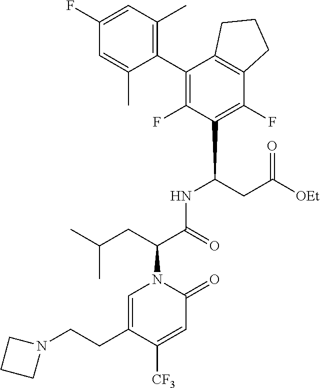 [SMILES: CCOC(=O)C[C@@H](NC(=O)[C@H](CC(C)C)n1cc(CCN2CCC2)c(C(F)(F)F)cc1=O)c1c(F)c2c(c(-c3c(C)cc(F)cc3C)c1F)CCC2] |
| | 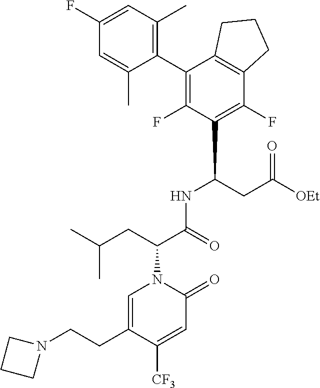 [SMILES: CCOC(=O)C[C@@H](NC(=O)[C@@H](CC(C)C)n1cc(CCN2CCC2)c(C(F)(F)F)cc1=O)c1c(F)c2c(c(-c3c(C)cc(F)cc3C)c1F)CCC2] |
| 270 | 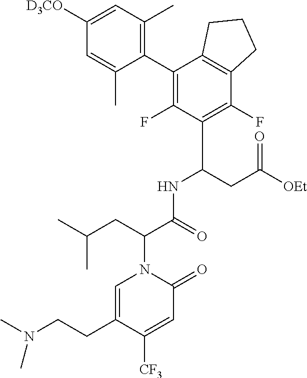 |
| | 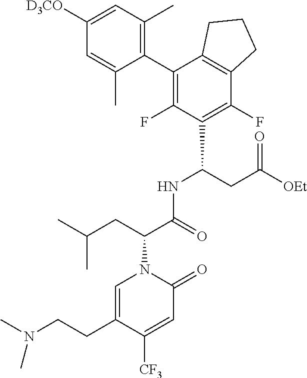 |
| | 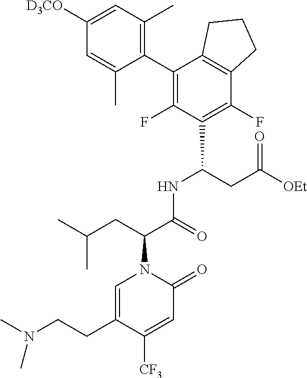 |
| | 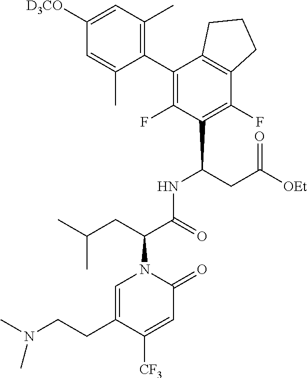 |
| | 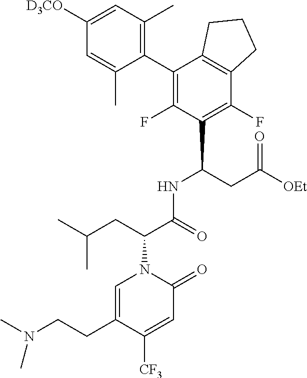 |
| 271 | 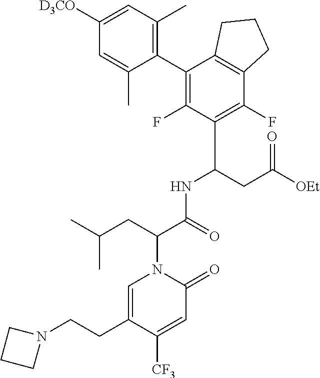 |
| | 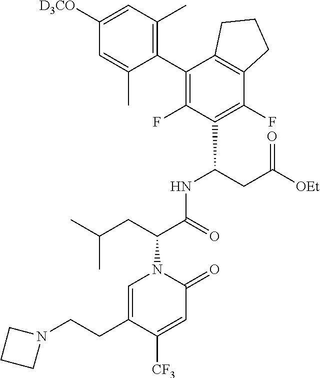 |
| | 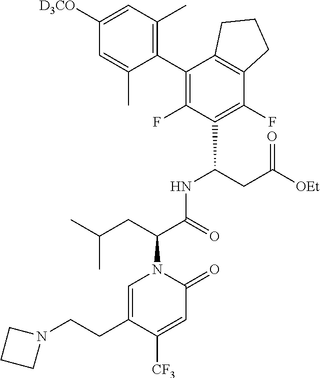 |
| | 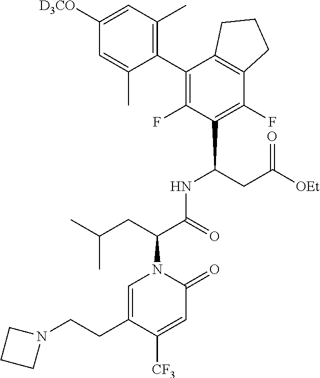 |
| | 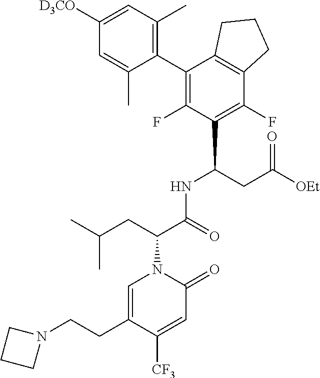 |
| 272 | 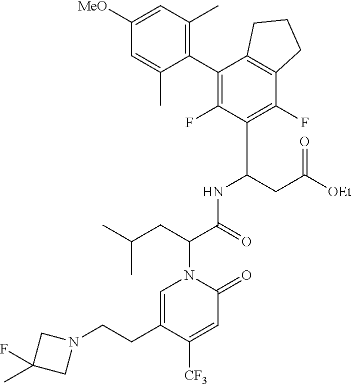 [SMILES: CCOC(=O)CC(NC(=O)C(CC(C)C)n1cc(CCN2CC(C)(F)C2)c(C(F)(F)F)cc1=O)c1c(F)c2c(c(-c3c(C)cc(OC)cc3C)c1F)CCC2] |
| | 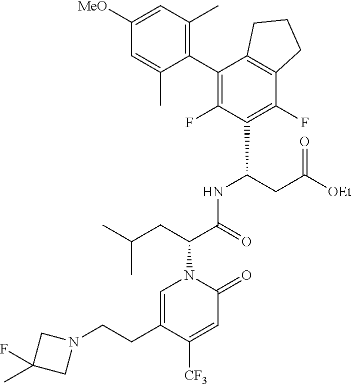 [SMILES: CCOC(=O)C[C@H](NC(=O)[C@@H](CC(C)C)n1cc(CCN2CC(C)(F)C2)c(C(F)(F)F)cc1=O)c1c(F)c2c(c(-c3c(C)cc(OC)cc3C)c1F)CCC2] |
| | 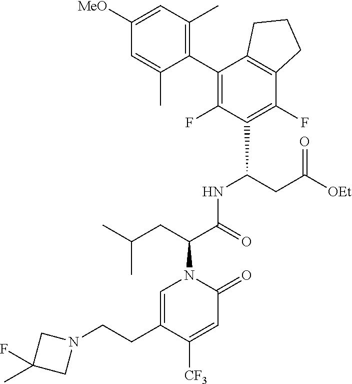 [SMILES: CCOC(=O)C[C@H](NC(=O)[C@H](CC(C)C)n1cc(CCN2CC(C)(F)C2)c(C(F)(F)F)cc1=O)c1c(F)c2c(c(-c3c(C)cc(OC)cc3C)c1F)CCC2] |
| | 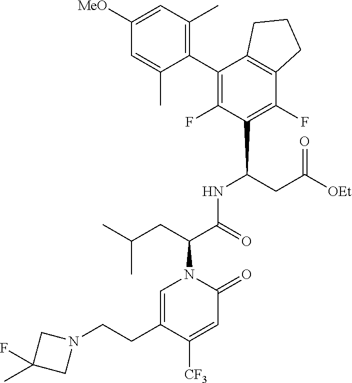 [SMILES: CCOC(=O)C[C@@H](NC(=O)[C@H](CC(C)C)n1cc(CCN2CC(C)(F)C2)c(C(F)(F)F)cc1=O)c1c(F)c2c(c(-c3c(C)cc(OC)cc3C)c1F)CCC2] |
| | 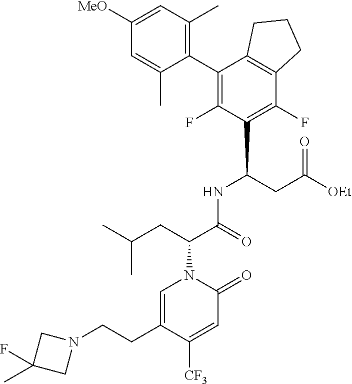 [SMILES: CCOC(=O)C[C@@H](NC(=O)[C@@H](CC(C)C)n1cc(CCN2CC(C)(F)C2)c(C(F)(F)F)cc1=O)c1c(F)c2c(c(-c3c(C)cc(OC)cc3C)c1F)CCC2] |
| 273 | 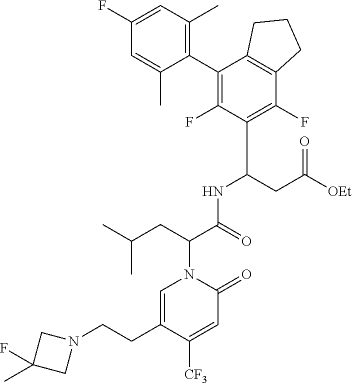 [SMILES: CCOC(=O)CC(NC(=O)C(CC(C)C)n1cc(CCN2CC(C)(F)C2)c(C(F)(F)F)cc1=O)c1c(F)c2c(c(-c3c(C)cc(F)cc3C)c1F)CCC2] |
| | 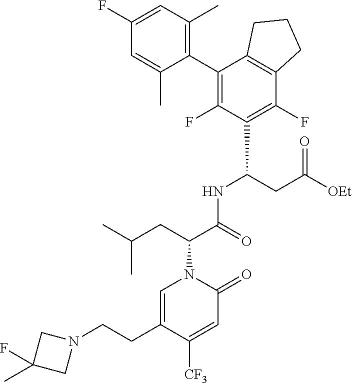 [SMILES: CCOC(=O)C[C@H](NC(=O)[C@@H](CC(C)C)n1cc(CCN2CC(C)(F)C2)c(C(F)(F)F)cc1=O)c1c(F)c2c(c(-c3c(C)cc(F)cc3C)c1F)CCC2] |
| | 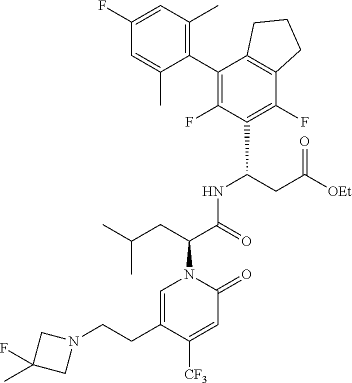 [SMILES: CCOC(=O)C[C@H](NC(=O)[C@H](CC(C)C)n1cc(CCN2CC(C)(F)C2)c(C(F)(F)F)cc1=O)c1c(F)c2c(c(-c3c(C)cc(F)cc3C)c1F)CCC2] |
| | 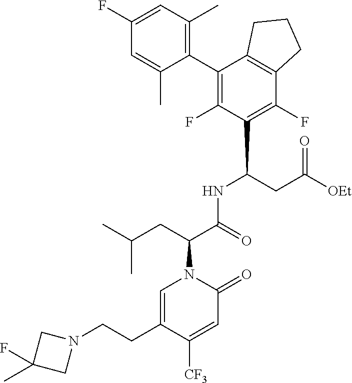 [SMILES: CCOC(=O)C[C@@H](NC(=O)[C@H](CC(C)C)n1cc(CCN2CC(C)(F)C2)c(C(F)(F)F)cc1=O)c1c(F)c2c(c(-c3c(C)cc(F)cc3C)c1F)CCC2] |
| | 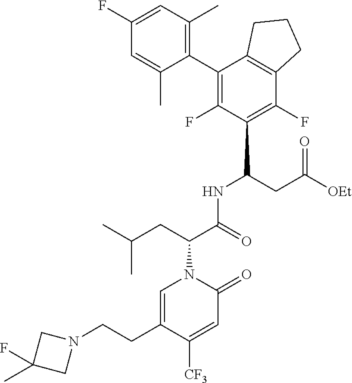 |
| 274 | 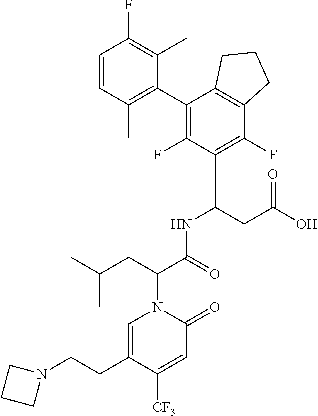 [SMILES: Cc1ccc(F)c(C)c1-c1c(F)c(C(CC(=O)O)NC(=O)C(CC(C)C)n2cc(CCN3CCC3)c(C(F)(F)F)cc2=O)c(F)c2c1CCC2] |
| | 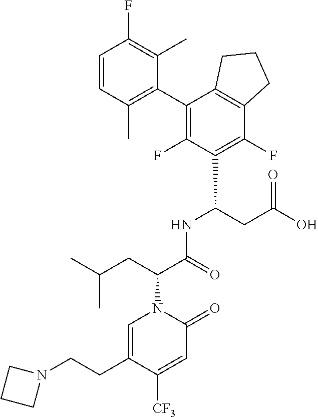 [SMILES: Cc1ccc(F)c(C)c1-c1c(F)c([C@H](CC(=O)O)NC(=O)[C@@H](CC(C)C)n2cc(CCN3CCC3)c(C(F)(F)F)cc2=O)c(F)c2c1CCC2] |
| | 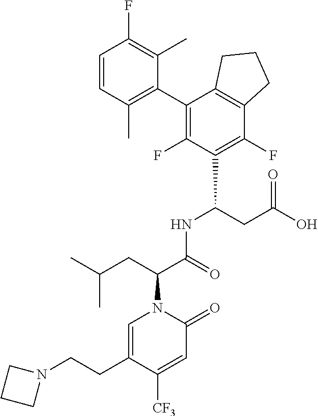 [SMILES: Cc1ccc(F)c(C)c1-c1c(F)c([C@H](CC(=O)O)NC(=O)[C@H](CC(C)C)n2cc(CCN3CCC3)c(C(F)(F)F)cc2=O)c(F)c2c1CCC2] |
| | 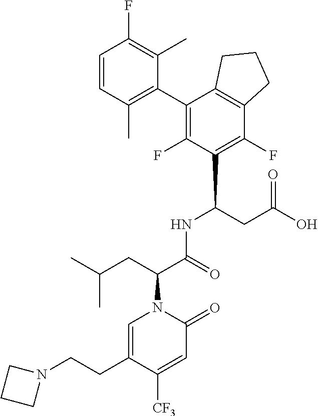 [SMILES: Cc1ccc(F)c(C)c1-c1c(F)c([C@@H](CC(=O)O)NC(=O)[C@H](CC(C)C)n2cc(CCN3CCC3)c(C(F)(F)F)cc2=O)c(F)c2c1CCC2] |
| | 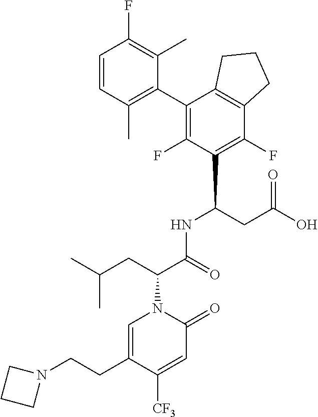 [SMILES: Cc1ccc(F)c(C)c1-c1c(F)c([C@@H](CC(=O)O)NC(=O)[C@@H](CC(C)C)n2cc(CCN3CCC3)c(C(F)(F)F)cc2=O)c(F)c2c1CCC2] |
| 275 | 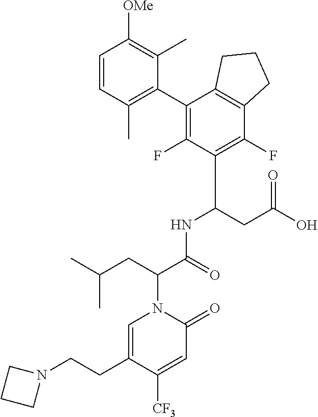 [SMILES: COc1ccc(C)c(-c2c(F)c(C(CC(=O)O)NC(=O)C(CC(C)C)n3cc(CCN4CCC4)c(C(F)(F)F)cc3=O)c(F)c3c2CCC3)c1C] |
| | 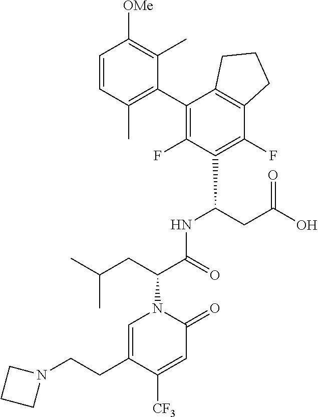 [SMILES: COc1ccc(C)c(-c2c(F)c([C@H](CC(=O)O)NC(=O)[C@@H](CC(C)C)n3cc(CCN4CCC4)c(C(F)(F)F)cc3=O)c(F)c3c2CCC3)c1C] |
| | 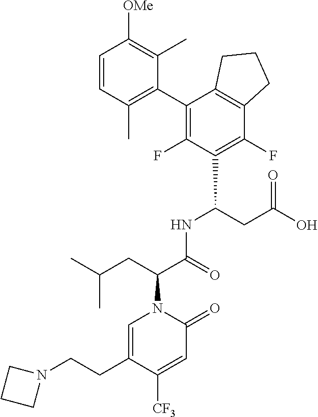 [SMILES: COc1ccc(C)c(-c2c(F)c([C@H](CC(=O)O)NC(=O)[C@H](CC(C)C)n3cc(CCN4CCC4)c(C(F)(F)F)cc3=O)c(F)c3c2CCC3)c1C] |
| | 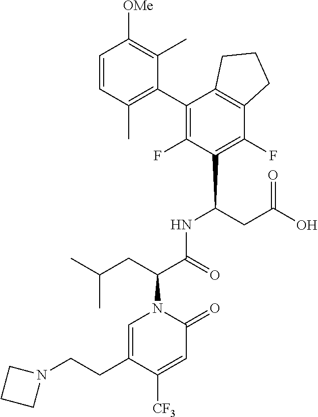 [SMILES: COc1ccc(C)c(-c2c(F)c([C@@H](CC(=O)O)NC(=O)[C@H](CC(C)C)n3cc(CCN4CCC4)c(C(F)(F)F)cc3=O)c(F)c3c2CCC3)c1C] |
| | 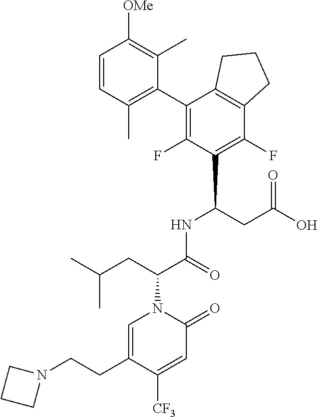 [SMILES: COc1ccc(C)c(-c2c(F)c([C@@H](CC(=O)O)NC(=O)[C@@H](CC(C)C)n3cc(CCN4CCC4)c(C(F)(F)F)cc3=O)c(F)c3c2CCC3)c1C] |
| 276 | 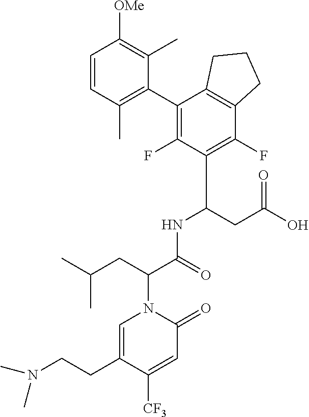 [SMILES: COc1ccc(C)c(-c2c(F)c(C(CC(=O)O)NC(=O)C(CC(C)C)n3cc(CCN(C)C)c(C(F)(F)F)cc3=O)c(F)c3c2CCC3)c1C] |
| | 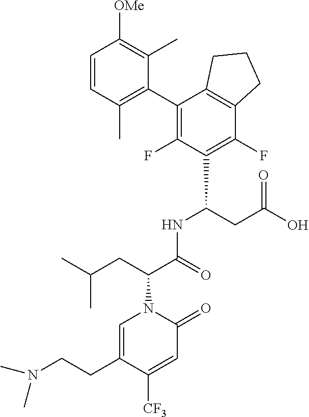 [SMILES: COc1ccc(C)c(-c2c(F)c([C@H](CC(=O)O)NC(=O)[C@@H](CC(C)C)n3cc(CCN(C)C)c(C(F)(F)F)cc3=O)c(F)c3c2CCC3)c1C] |
| | 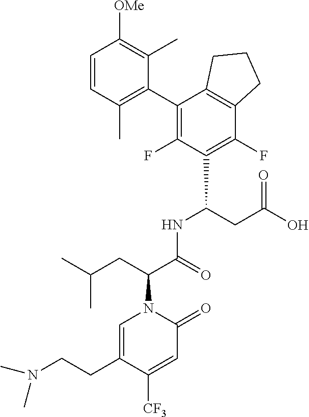 [SMILES: COc1ccc(C)c(-c2c(F)c([C@H](CC(=O)O)NC(=O)[C@H](CC(C)C)n3cc(CCN(C)C)c(C(F)(F)F)cc3=O)c(F)c3c2CCC3)c1C] |
| | 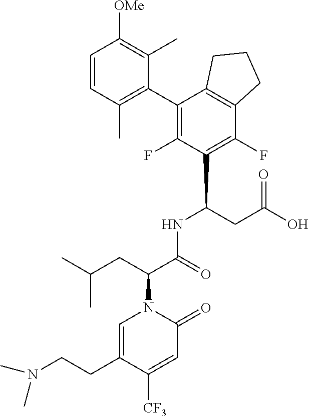 [SMILES: COc1ccc(C)c(-c2c(F)c([C@@H](CC(=O)O)NC(=O)[C@H](CC(C)C)n3cc(CCN(C)C)c(C(F)(F)F)cc3=O)c(F)c3c2CCC3)c1C] |
| | 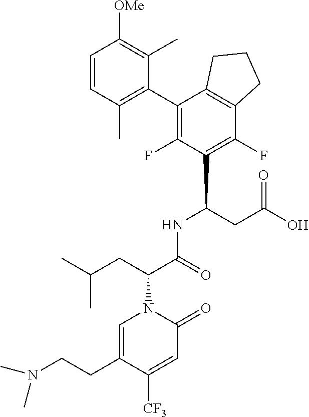 [SMILES: COc1ccc(C)c(-c2c(F)c([C@@H](CC(=O)O)NC(=O)[C@@H](CC(C)C)n3cc(CCN(C)C)c(C(F)(F)F)cc3=O)c(F)c3c2CCC3)c1C] |
| 277 | 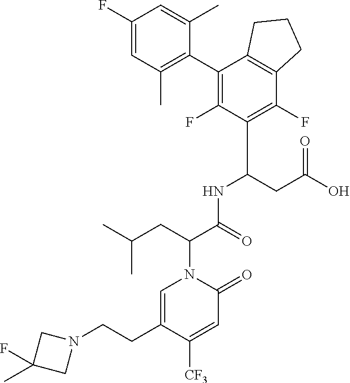 [SMILES: Cc1cc(F)cc(C)c1-c1c(F)c(C(CC(=O)O)NC(=O)C(CC(C)C)n2cc(CCN3CC(C)(F)C3)c(C(F)(F)F)cc2=O)c(F)c2c1CCC2] |
| | 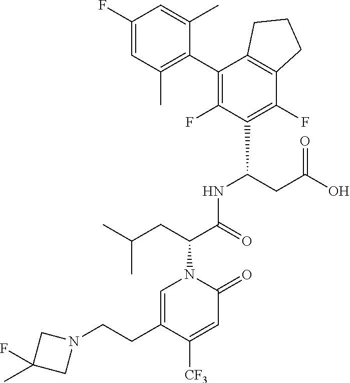 [SMILES: Cc1cc(F)cc(C)c1-c1c(F)c([C@H](CC(=O)O)NC(=O)[C@@H](CC(C)C)n2cc(CCN3CC(C)(F)C3)c(C(F)(F)F)cc2=O)c(F)c2c1CCC2] |
| | 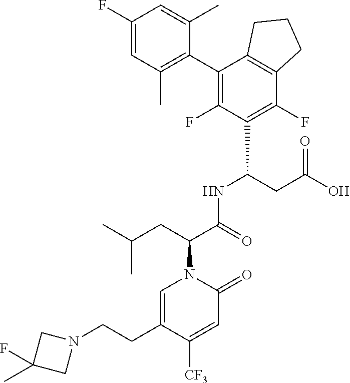 [SMILES: Cc1cc(F)cc(C)c1-c1c(F)c([C@H](CC(=O)O)NC(=O)[C@H](CC(C)C)n2cc(CCN3CC(C)(F)C3)c(C(F)(F)F)cc2=O)c(F)c2c1CCC2] |
| | 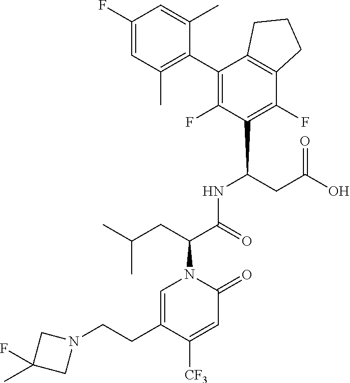 [SMILES: Cc1cc(F)cc(C)c1-c1c(F)c([C@@H](CC(=O)O)NC(=O)[C@H](CC(C)C)n2cc(CCN3CC(C)(F)C3)c(C(F)(F)F)cc2=O)c(F)c2c1CCC2] |
| | 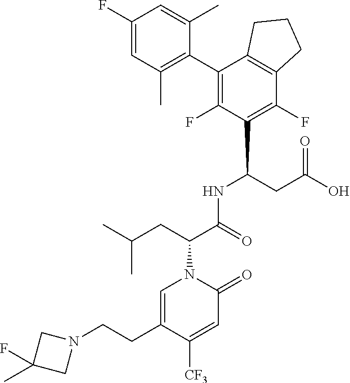 [SMILES: Cc1cc(F)cc(C)c1-c1c(F)c([C@@H](CC(=O)O)NC(=O)[C@@H](CC(C)C)n2cc(CCN3CC(C)(F)C3)c(C(F)(F)F)cc2=O)c(F)c2c1CCC2] |
| 278 | 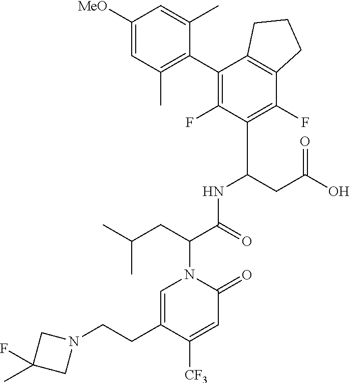 [SMILES: COc1cc(C)c(-c2c(F)c(C(CC(=O)O)NC(=O)C(CC(C)C)n3cc(CCN4CC(C)(F)C4)c(C(F)(F)F)cc3=O)c(F)c3c2CCC3)c(C)c1] / |
| | 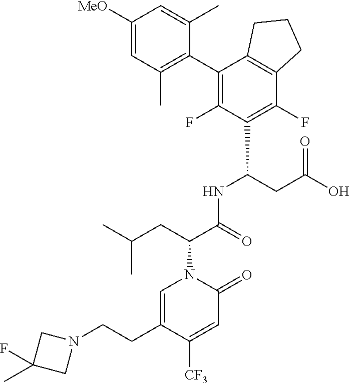 [SMILES: COc1cc(C)c(-c2c(F)c([C@H](CC(=O)O)NC(=O)[C@@H](CC(C)C)n3cc(CCN4CC(C)(F)C4)c(C(F)(F)F)cc3=O)c(F)c3c2CCC3)c(C)c1] |
| | 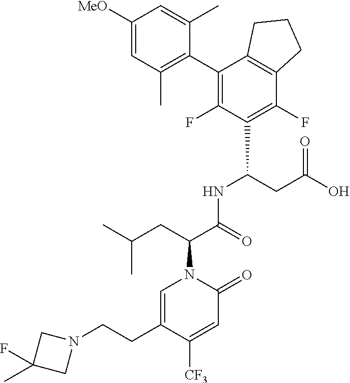 [SMILES: COc1cc(C)c(-c2c(F)c([C@H](CC(=O)O)NC(=O)[C@H](CC(C)C)n3cc(CCN4CC(C)(F)C4)c(C(F)(F)F)cc3=O)c(F)c3c2CCC3)c(C)c1] |
| | 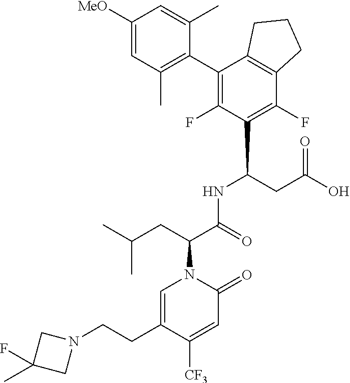 [SMILES: COc1cc(C)c(-c2c(F)c([C@@H](CC(=O)O)NC(=O)[C@H](CC(C)C)n3cc(CCN4CC(C)(F)C4)c(C(F)(F)F)cc3=O)c(F)c3c2CCC3)c(C)c1] |
| | 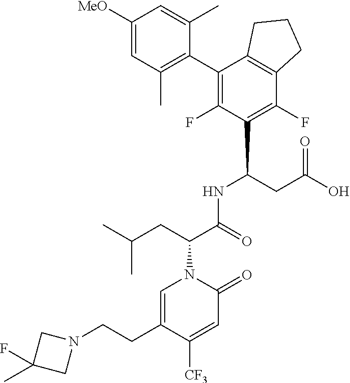 [SMILES: COc1cc(C)c(-c2c(F)c([C@@H](CC(=O)O)NC(=O)[C@@H](CC(C)C)n3cc(CCN4CC(C)(F)C4)c(C(F)(F)F)cc3=O)c(F)c3c2CCC3)c(C)c1] |

TABLE E-4

| | |
|---|---|
| 279 | 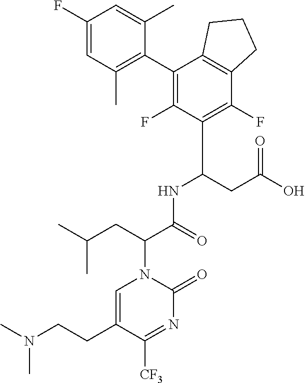 [SMILES: Cc1cc(F)cc(C)c1-c1c(F)c(C(CC(=O)O)NC(=O)C(CC(C)C)n2cc(CCN(C)C)c(C(F)(F)F)nc2=O)c(F)c2c1CCC2] |
| | 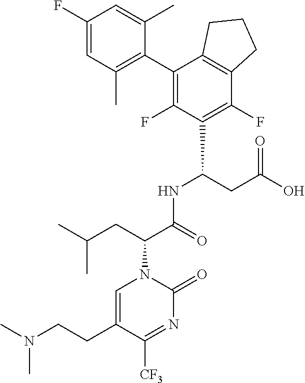 [SMILES: Cc1cc(F)cc(C)c1-c1c(F)c([C@H](CC(=O)O)NC(=O)[C@@H](CC(C)C)n2cc(CCN(C)C)c(C(F)(F)F)nc2=O)c(F)c2c1CCC2] |
| | 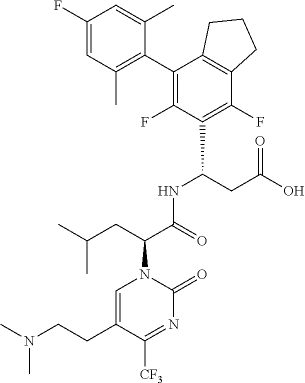 [SMILES: Cc1cc(F)cc(C)c1-c1c(F)c([C@H](CC(=O)O)NC(=O)[C@H](CC(C)C)n2cc(CCN(C)C)c(C(F)(F)F)nc2=O)c(F)c2c1CCC2] |
| | 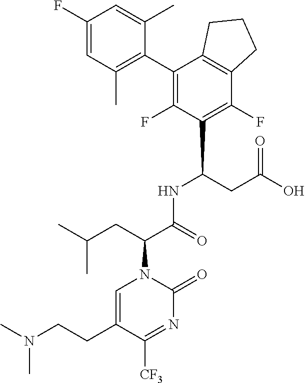 [SMILES: Cc1cc(F)cc(C)c1-c1c(F)c([C@@H](CC(=O)O)NC(=O)[C@H](CC(C)C)n2cc(CCN(C)C)c(C(F)(F)F)nc2=O)c(F)c2c1CCC2] |
| | 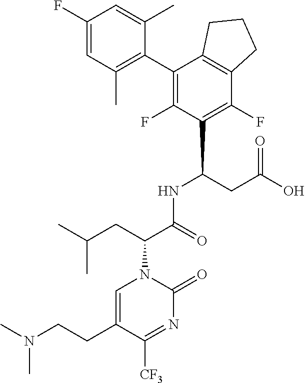 [SMILES: Cc1cc(F)cc(C)c1-c1c(F)c([C@@H](CC(=O)O)NC(=O)[C@@H](CC(C)C)n2cc(CCN(C)C)c(C(F)(F)F)nc2=O)c(F)c2c1CCC2] |
| 280 | 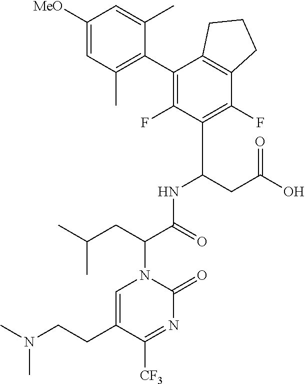 [SMILES: COc1cc(C)c(-c2c(F)c(C(CC(=O)O)NC(=O)C(CC(C)C)n3cc(CCN(C)C)c(C(F)(F)F)nc3=O)c(F)c3c2CCC3)c(C)c1] |
| | 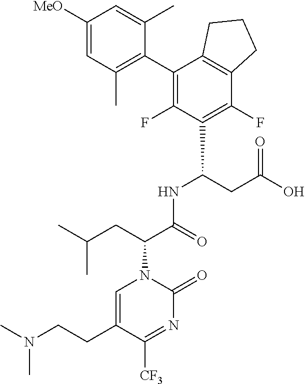 [SMILES: COc1cc(C)c(-c2c(F)c([C@H](CC(=O)O)NC(=O)[C@@H](CC(C)C)n3cc(CCN(C)C)c(C(F)(F)F)nc3=O)c(F)c3c2CCC3)c(C)c1] |
| | 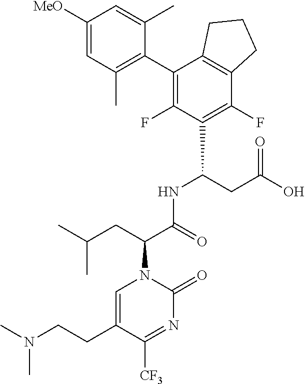 [SMILES: COc1cc(C)c(-c2c(F)c([C@H](CC(=O)O)NC(=O)[C@H](CC(C)C)n3cc(CCN(C)C)c(C(F)(F)F)nc3=O)c(F)c3c2CCC3)c(C)c1] |
| | 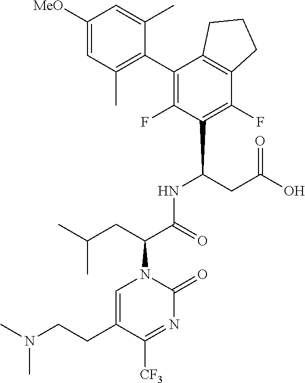 [SMILES: COc1cc(C)c(-c2c(F)c([C@@H](CC(=O)O)NC(=O)[C@H](CC(C)C)n3cc(CCN(C)C)c(C(F)(F)F)nc3=O)c(F)c3c2CCC3)c(C)c1] |
| | 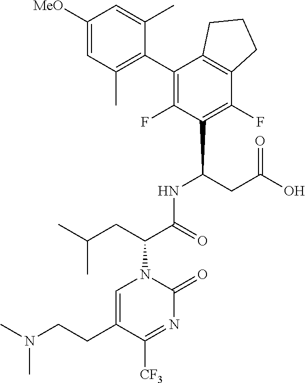 [SMILES: COc1cc(C)c(-c2c(F)c([C@@H](CC(=O)O)NC(=O)[C@@H](CC(C)C)n3cc(CCN(C)C)c(C(F)(F)F)nc3=O)c(F)c3c2CCC3)c(C)c1] |
| 281 | 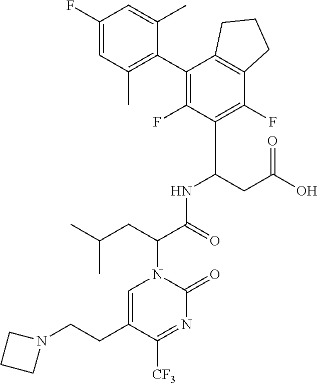 [SMILES: Cc1cc(F)cc(C)c1-c1c(F)c(C(CC(=O)O)NC(=O)C(CC(C)C)n2cc(CCN3CCC3)c(C(F)(F)F)nc2=O)c(F)c2c1CCC2] |
| | 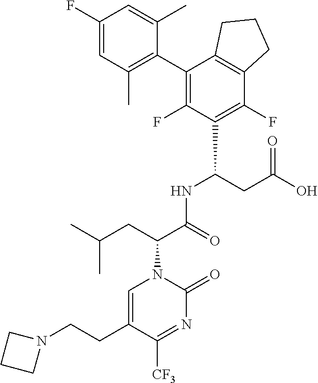 [SMILES: Cc1cc(F)cc(C)c1-c1c(F)c([C@H](CC(=O)O)NC(=O)[C@@H](CC(C)C)n2cc(CCN3CCC3)c(C(F)(F)F)nc2=O)c(F)c2c1CCC2] |
| | 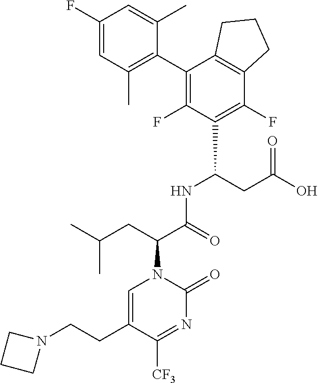 [SMILES: Cc1cc(F)cc(C)c1-c1c(F)c([C@H](CC(=O)O)NC(=O)[C@H](CC(C)C)n2cc(CCN3CCC3)c(C(F)(F)F)nc2=O)c(F)c2c1CCC2] |
| | 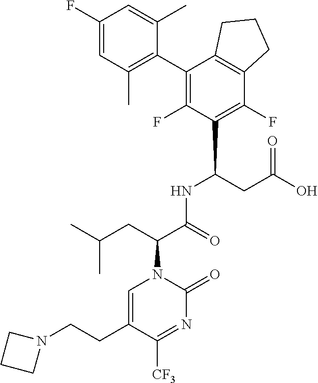 [SMILES: Cc1cc(F)cc(C)c1-c1c(F)c([C@@H](CC(=O)O)NC(=O)[C@H](CC(C)C)n2cc(CCN3CCC3)c(C(F)(F)F)nc2=O)c(F)c2c1CCC2] |
| | 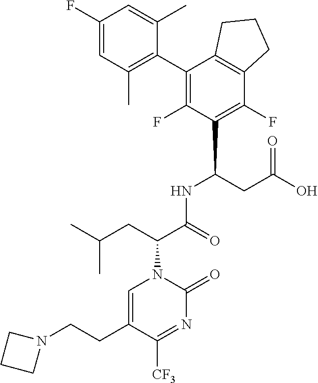 [SMILES: Cc1cc(F)cc(C)c1-c1c(F)c([C@@H](CC(=O)O)NC(=O)[C@@H](CC(C)C)n2cc(CCN3CCC3)c(C(F)(F)F)nc2=O)c(F)c2c1CCC2] |
| 282 | 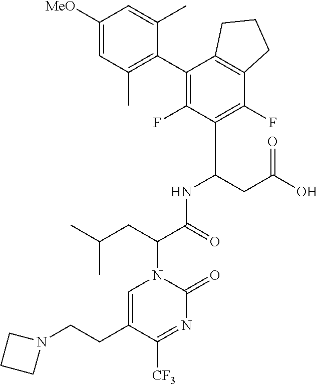 [SMILES: COc1cc(C)c(-c2c(F)c(C(CC(=O)O)NC(=O)C(CC(C)C)n3cc(CCN4CCC4)c(C(F)(F)F)nc3=O)c(F)c3c2CCC3)c(C)c1] |
| | 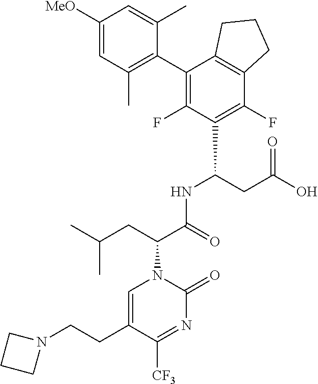 [SMILES: COc1cc(C)c(-c2c(F)c([C@H](CC(=O)O)NC(=O)[C@@H](CC(C)C)n3cc(CCN4CCC4)c(C(F)(F)F)nc3=O)c(F)c3c2CCC3)c(C)c1] |
| | 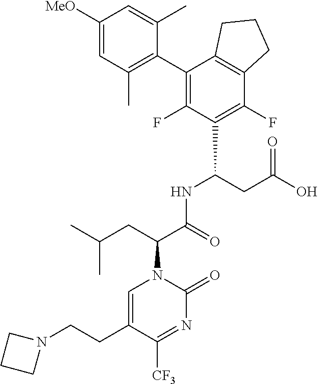 [SMILES: COc1cc(C)c(-c2c(F)c([C@H](CC(=O)O)NC(=O)[C@H](CC(C)C)n3cc(CCN4CCC4)c(C(F)(F)F)nc3=O)c(F)c3c2CCC3)c(C)c1] |
| | 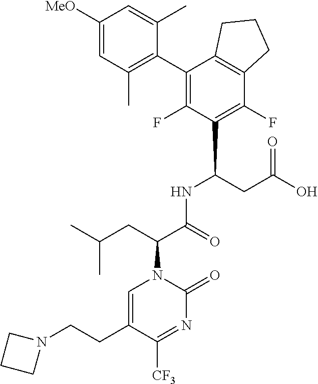 [SMILES: COc1cc(C)c(-c2c(F)c([C@@H](CC(=O)O)NC(=O)[C@H](CC(C)C)n3cc(CCN4CCC4)c(C(F)(F)F)nc3=O)c(F)c3c2CCC3)c(C)c1] |
| | 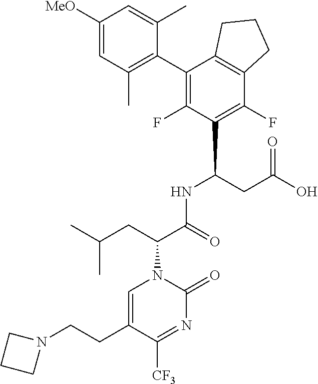 [SMILES: COc1cc(C)c(-c2c(F)c([C@@H](CC(=O)O)NC(=O)[C@@H](CC(C)C)n3cc(CCN4CCC4)c(C(F)(F)F)nc3=O)c(F)c3c2CCC3)c(C)c1] |
| 283 | 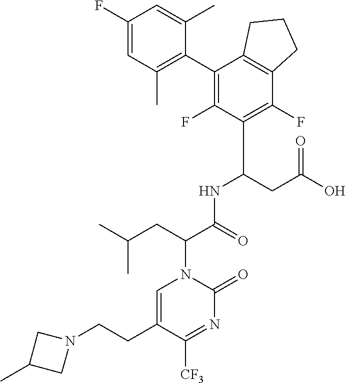 [SMILES: Cc1cc(F)cc(C)c1-c1c(F)c(C(CC(=O)O)NC(=O)C(CC(C)C)n2cc(CCN3CC(C)C3)c(C(F)(F)F)nc2=O)c(F)c2c1CCC2] |
| | 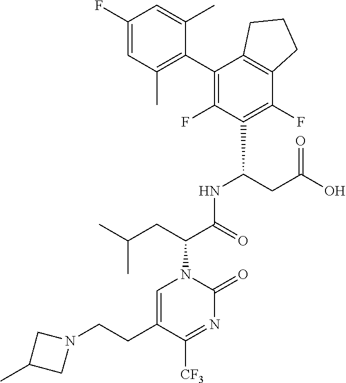 [SMILES: Cc1cc(F)cc(C)c1-c1c(F)c([C@H](CC(=O)O)NC(=O)[C@@H](CC(C)C)n2cc(CCN3CC(C)C3)c(C(F)(F)F)nc2=O)c(F)c2c1CCC2] |
| | 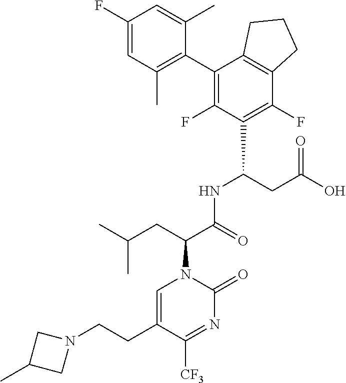 [SMILES: Cc1cc(F)cc(C)c1-c1c(F)c([C@H](CC(=O)O)NC(=O)[C@H](CC(C)C)n2cc(CCN3CC(C)C3)c(C(F)(F)F)nc2=O)c(F)c2c1CCC2] |
| | 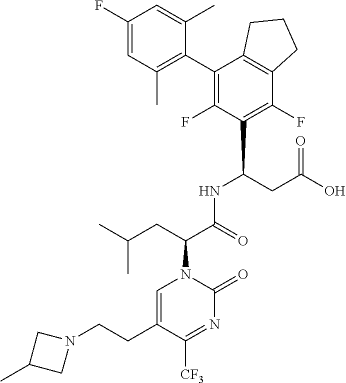 [SMILES: Cc1cc(F)cc(C)c1-c1c(F)c([C@@H](CC(=O)O)NC(=O)[C@H](CC(C)C)n2cc(CCN3CC(C)C3)c(C(F)(F)F)nc2=O)c(F)c2c1CCC2] |
| | 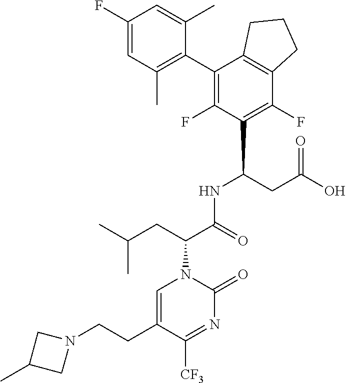 [SMILES: Cc1cc(F)cc(C)c1-c1c(F)c([C@@H](CC(=O)O)NC(=O)[C@@H](CC(C)C)n2cc(CCN3CC(C)C3)c(C(F)(F)F)nc2=O)c(F)c2c1CCC2] |
| 284 | 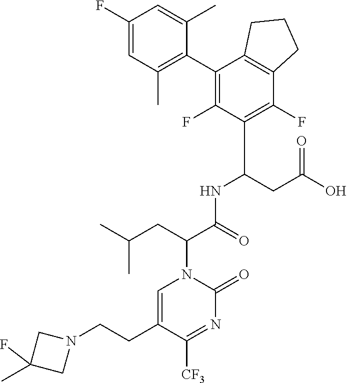 [SMILES: Cc1cc(F)cc(C)c1-c1c(F)c(C(CC(=O)O)NC(=O)C(CC(C)C)n2cc(CCN3CC(C)(F)C3)c(C(F)(F)F)nc2=O)c(F)c2c1CCC2] |
| | 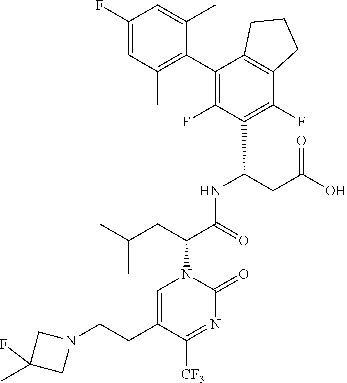 [SMILES: Cc1cc(F)cc(C)c1-c1c(F)c([C@H](CC(=O)O)NC(=O)[C@@H](CC(C)C)n2cc(CCN3CC(C)(F)C3)c(C(F)(F)F)nc2=O)c(F)c2c1CCC2] |
| | 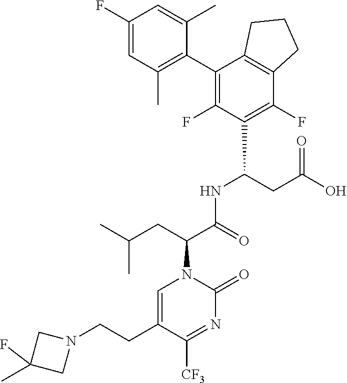 [SMILES: Cc1cc(F)cc(C)c1-c1c(F)c([C@H](CC(=O)O)NC(=O)[C@H](CC(C)C)n2cc(CCN3CC(C)(F)C3)c(C(F)(F)F)nc2=O)c(F)c2c1CCC2] |
| | 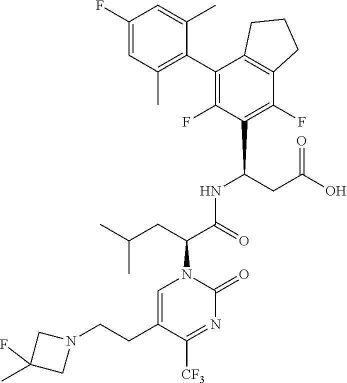 [SMILES: Cc1cc(F)cc(C)c1-c1c(F)c([C@@H](CC(=O)O)NC(=O)[C@H](CC(C)C)n2cc(CCN3CC(C)(F)C3)c(C(F)(F)F)nc2=O)c(F)c2c1CCC2] |
| | 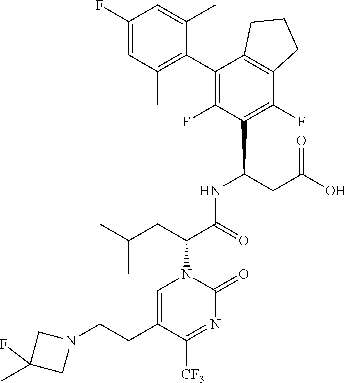 [SMILES: Cc1cc(F)cc(C)c1-c1c(F)c([C@@H](CC(=O)O)NC(=O)[C@@H](CC(C)C)n2cc(CCN3CC(C)(F)C3)c(C(F)(F)F)nc2=O)c(F)c2c1CCC2] |
| 285 | 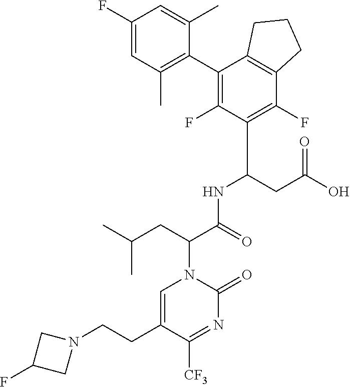 [SMILES: Cc1cc(F)cc(C)c1-c1c(F)c(C(CC(=O)O)NC(=O)C(CC(C)C)n2cc(CCN3CC(F)C3)c(C(F)(F)F)nc2=O)c(F)c2c1CCC2] |
| | 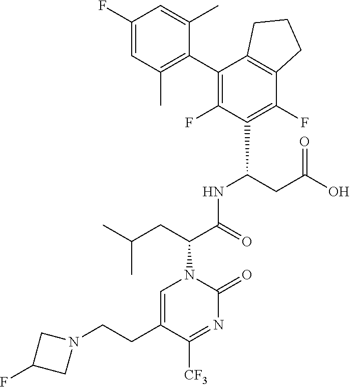 [SMILES: Cc1cc(F)cc(C)c1-c1c(F)c([C@H](CC(=O)O)NC(=O)[C@@H](CC(C)C)n2cc(CCN3CC(F)C3)c(C(F)(F)F)nc2=O)c(F)c2c1CCC2] |
| | 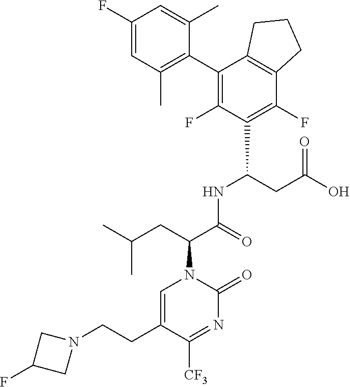 [SMILES: Cc1cc(F)cc(C)c1-c1c(F)c([C@H](CC(=O)O)NC(=O)[C@H](CC(C)C)n2cc(CCN3CC(F)C3)c(C(F)(F)F)nc2=O)c(F)c2c1CCC2] |
| | 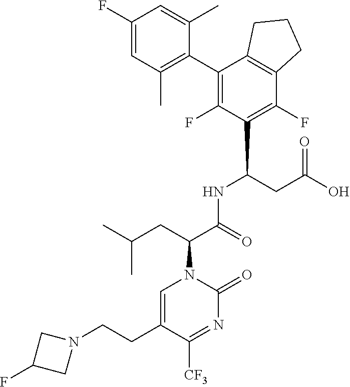 [SMILES: Cc1cc(F)cc(C)c1-c1c(F)c([C@@H](CC(=O)O)NC(=O)[C@H](CC(C)C)n2cc(CCN3CC(F)C3)c(C(F)(F)F)nc2=O)c(F)c2c1CCC2] |
| | 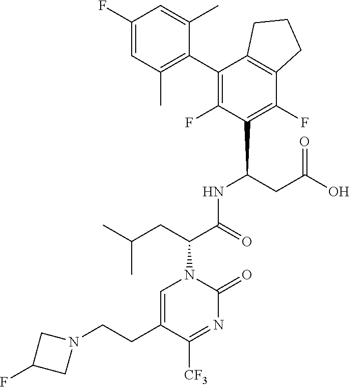 [SMILES: Cc1cc(F)cc(C)c1-c1c(F)c([C@@H](CC(=O)O)NC(=O)[C@@H](CC(C)C)n2cc(CCN3CC(F)C3)c(C(F)(F)F)nc2=O)c(F)c2c1CCC2] |
| 286 | 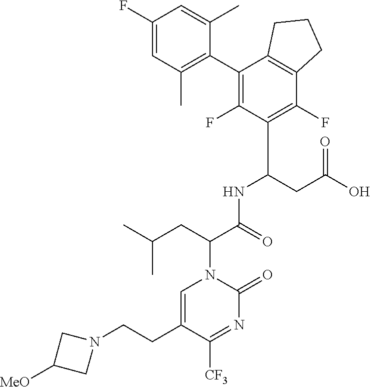 [SMILES: COC1CN(CCc2cn(C(CC(C)C)C(=O)NC(CC(=O)O)c3c(F)c4c(c(-c5c(C)cc(F)cc5C)c3F)CCC4)c(=O)nc2C(F)(F)F)C1] |
| | 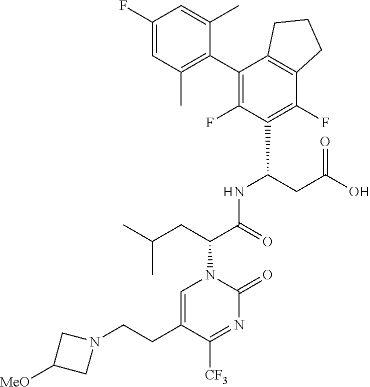 [SMILES: COC1CN(CCc2cn([C@H](CC(C)C)C(=O)N[C@@H](CC(=O)O)c3c(F)c4c(c(-c5c(C)cc(F)cc5C)c3F)CCC4)c(=O)nc2C(F)(F)F)C1] |
| | 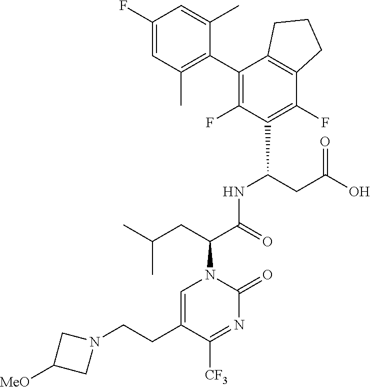 [SMILES: COC1CN(CCc2cn([C@@H](CC(C)C)C(=O)N[C@@H](CC(=O)O)c3c(F)c4c(c(-c5c(C)cc(F)cc5C)c3F)CCC4)c(=O)nc2C(F)(F)F)C1] |
| | 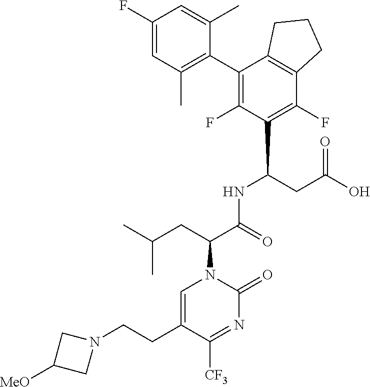 [SMILES: COC1CN(CCc2cn([C@@H](CC(C)C)C(=O)N[C@H](CC(=O)O)c3c(F)c4c(c(-c5c(C)cc(F)cc5C)c3F)CCC4)c(=O)nc2C(F)(F)F)C1] |
| | 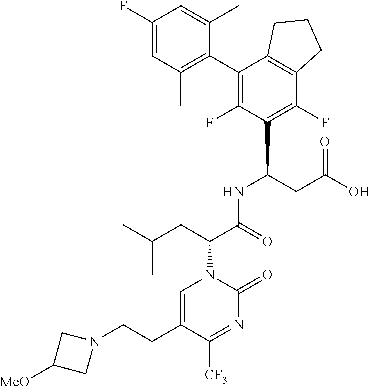 [SMILES: COC1CN(CCc2cn([C@H](CC(C)C)C(=O)N[C@H](CC(=O)O)c3c(F)c4c(c(-c5c(C)cc(F)cc5C)c3F)CCC4)c(=O)nc2C(F)(F)F)C1] |
| 287 | 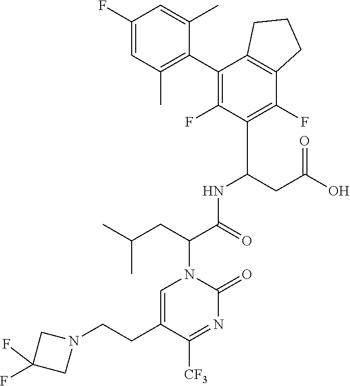 [SMILES: Cc1cc(F)cc(C)c1-c1c(F)c(C(CC(=O)O)NC(=O)C(CC(C)C)n2cc(CCN3CC(F)(F)C3)c(C(F)(F)F)nc2=O)c(F)c2c1CCC2] |
| | 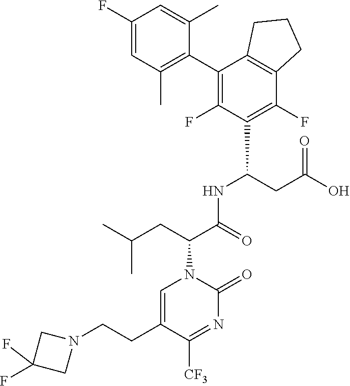 [SMILES: Cc1cc(F)cc(C)c1-c1c(F)c([C@H](CC(=O)O)NC(=O)[C@@H](CC(C)C)n2cc(CCN3CC(F)(F)C3)c(C(F)(F)F)nc2=O)c(F)c2c1CCC2] |
| | 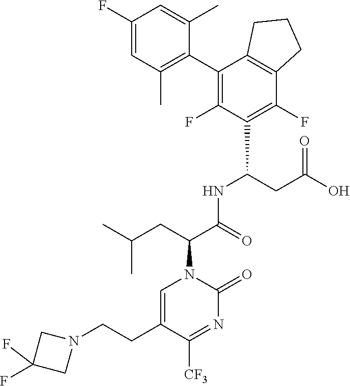 [SMILES: Cc1cc(F)cc(C)c1-c1c(F)c([C@H](CC(=O)O)NC(=O)[C@H](CC(C)C)n2cc(CCN3CC(F)(F)C3)c(C(F)(F)F)nc2=O)c(F)c2c1CCC2] |
| | 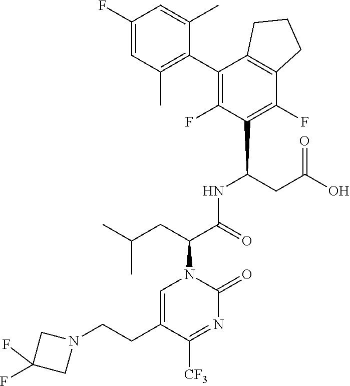 [SMILES: Cc1cc(F)cc(C)c1-c1c(F)c([C@@H](CC(=O)O)NC(=O)[C@H](CC(C)C)n2cc(CCN3CC(F)(F)C3)c(C(F)(F)F)nc2=O)c(F)c2c1CCC2] |
| | 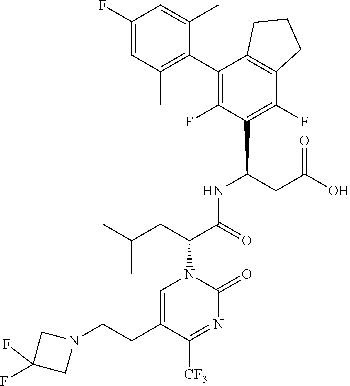 [SMILES: Cc1cc(F)cc(C)c1-c1c(F)c([C@@H](CC(=O)O)NC(=O)[C@@H](CC(C)C)n2cc(CCN3CC(F)(F)C3)c(C(F)(F)F)nc2=O)c(F)c2c1CCC2] |
| 288 | 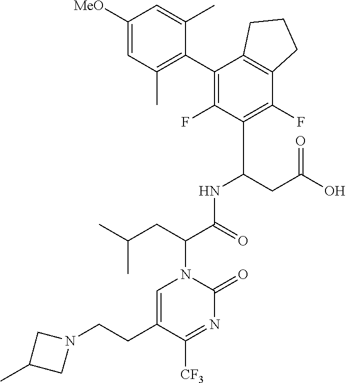 [SMILES: COc1cc(C)c(-c2c(F)c(C(CC(=O)O)NC(=O)C(CC(C)C)n3cc(CCN4CC(C)C4)c(C(F)(F)F)nc3=O)c(F)c3c2CCC3)c(C)c1] |
| | 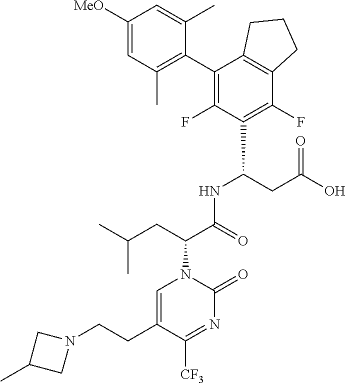 [SMILES: COc1cc(C)c(-c2c(F)c([C@H](CC(=O)O)NC(=O)[C@@H](CC(C)C)n3cc(CCN4CC(C)C4)c(C(F)(F)F)nc3=O)c(F)c3c2CCC3)c(C)c1] |
| | 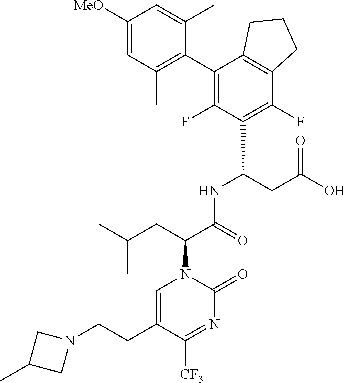 [SMILES: COc1cc(C)c(-c2c(F)c([C@H](CC(=O)O)NC(=O)[C@H](CC(C)C)n3cc(CCN4CC(C)C4)c(C(F)(F)F)nc3=O)c(F)c3c2CCC3)c(C)c1] |
| | 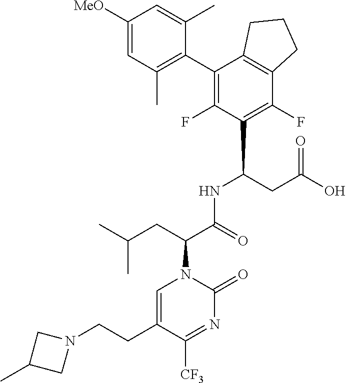 [SMILES: COc1cc(C)c(-c2c(F)c([C@@H](CC(=O)O)NC(=O)[C@H](CC(C)C)n3cc(CCN4CC(C)C4)c(C(F)(F)F)nc3=O)c(F)c3c2CCC3)c(C)c1] |
| | 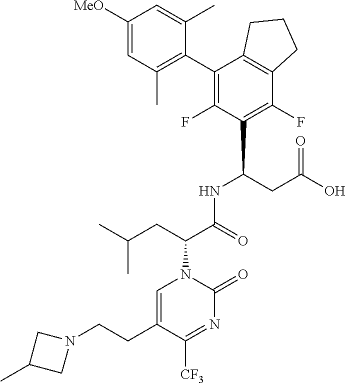 [SMILES: COc1cc(C)c(-c2c(F)c([C@@H](CC(=O)O)NC(=O)[C@@H](CC(C)C)n3cc(CCN4CC(C)C4)c(C(F)(F)F)nc3=O)c(F)c3c2CCC3)c(C)c1] |
| 289 | 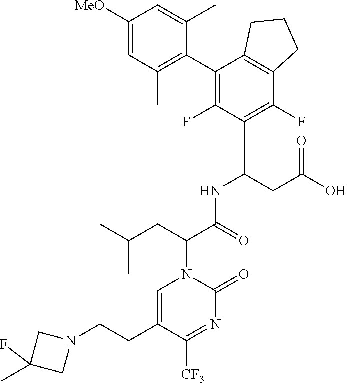 [SMILES: COc1cc(C)c(-c2c(F)c(C(CC(=O)O)NC(=O)C(CC(C)C)n3cc(CCN4CC(C)(F)C4)c(C(F)(F)F)nc3=O)c(F)c3c2CCC3)c(C)c1] |
| | 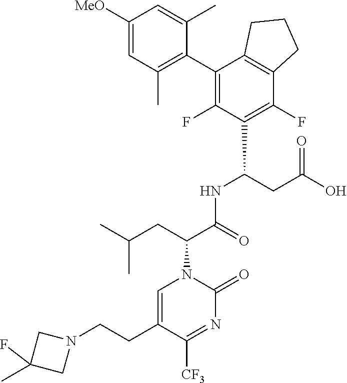 [SMILES: COc1cc(C)c(-c2c(F)c([C@H](CC(=O)O)NC(=O)[C@@H](CC(C)C)n3cc(CCN4CC(C)(F)C4)c(C(F)(F)F)nc3=O)c(F)c3c2CCC3)c(C)c1] |
| | 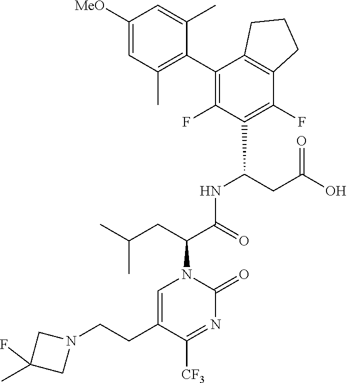 [SMILES: COc1cc(C)c(-c2c(F)c([C@H](CC(=O)O)NC(=O)[C@H](CC(C)C)n3cc(CCN4CC(C)(F)C4)c(C(F)(F)F)nc3=O)c(F)c3c2CCC3)c(C)c1] |
| | 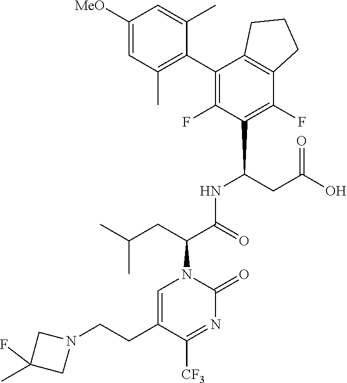 [SMILES: COc1cc(C)c(-c2c(F)c([C@@H](CC(=O)O)NC(=O)[C@H](CC(C)C)n3cc(CCN4CC(C)(F)C4)c(C(F)(F)F)nc3=O)c(F)c3c2CCC3)c(C)c1] |
| | 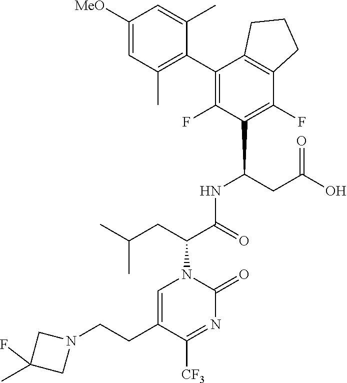 [SMILES: COc1cc(C)c(-c2c(F)c([C@@H](CC(=O)O)NC(=O)[C@@H](CC(C)C)n3cc(CCN4CC(C)(F)C4)c(C(F)(F)F)nc3=O)c(F)c3c2CCC3)c(C)c1] |
| 290 | 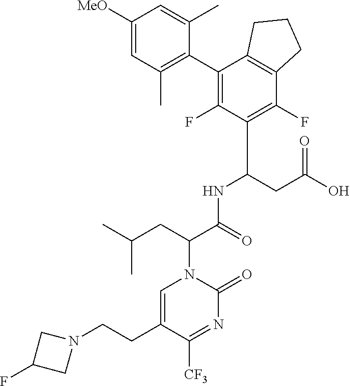 [SMILES: COc1cc(C)c(-c2c(F)c(C(CC(=O)O)NC(=O)C(CC(C)C)n3cc(CCN4CC(F)C4)c(C(F)(F)F)nc3=O)c(F)c3c2CCC3)c(C)c1] |
| | 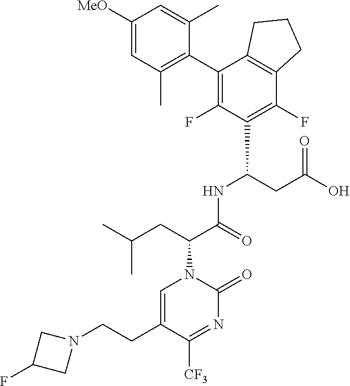 [SMILES: COc1cc(C)c(-c2c(F)c([C@H](CC(=O)O)NC(=O)[C@@H](CC(C)C)n3cc(CCN4CC(F)C4)c(C(F)(F)F)nc3=O)c(F)c3c2CCC3)c(C)c1] |
| | 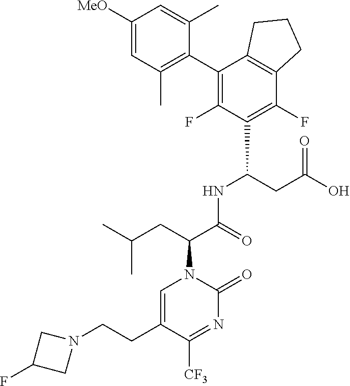 [SMILES: COc1cc(C)c(-c2c(F)c([C@H](CC(=O)O)NC(=O)[C@H](CC(C)C)n3cc(CCN4CC(F)C4)c(C(F)(F)F)nc3=O)c(F)c3c2CCC3)c(C)c1] |
| | 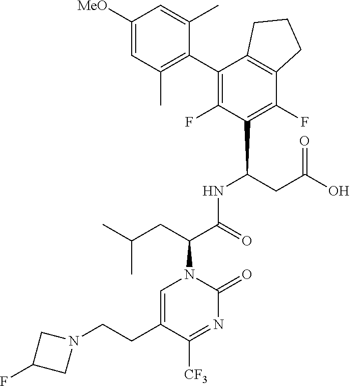 [SMILES: COc1cc(C)c(-c2c(F)c([C@@H](CC(=O)O)NC(=O)[C@H](CC(C)C)n3cc(CCN4CC(F)C4)c(C(F)(F)F)nc3=O)c(F)c3c2CCC3)c(C)c1] |
| | 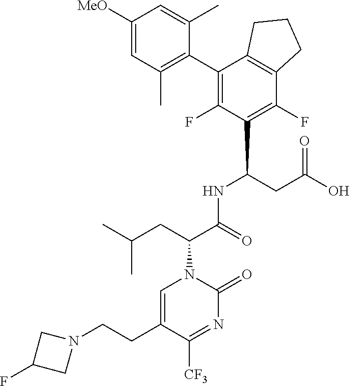 [SMILES: COc1cc(C)c(-c2c(F)c([C@@H](CC(=O)O)NC(=O)[C@@H](CC(C)C)n3cc(CCN4CC(F)C4)c(C(F)(F)F)nc3=O)c(F)c3c2CCC3)c(C)c1] |
| 291 | 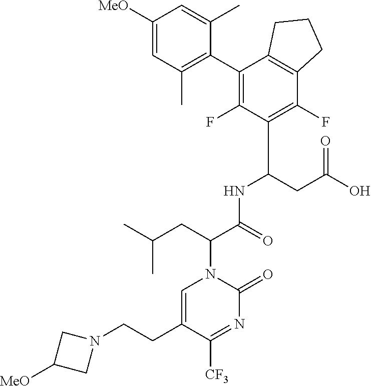 [SMILES: COc1cc(C)c(-c2c(F)c(C(CC(=O)O)NC(=O)C(CC(C)C)n3cc(CCN4CC(OC)C4)c(C(F)(F)F)nc3=O)c(F)c3c2CCC3)c(C)c1] |
| | 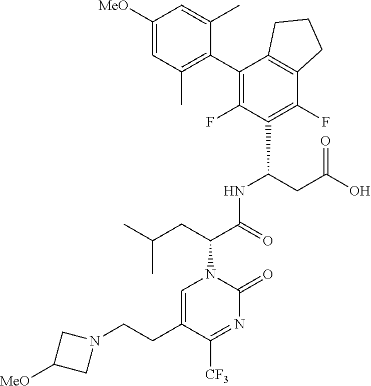 [SMILES: COc1cc(C)c(-c2c(F)c([C@H](CC(=O)O)NC(=O)[C@@H](CC(C)C)n3cc(CCN4CC(OC)C4)c(C(F)(F)F)nc3=O)c(F)c3c2CCC3)c(C)c1] |
| | 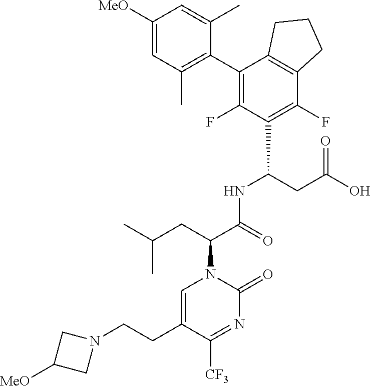 [SMILES: COc1cc(C)c(-c2c(F)c([C@H](CC(=O)O)NC(=O)[C@H](CC(C)C)n3cc(CCN4CC(OC)C4)c(C(F)(F)F)nc3=O)c(F)c3c2CCC3)c(C)c1] |
| | 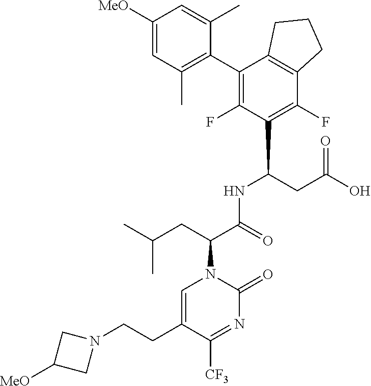 [SMILES: COc1cc(C)c(-c2c(F)c([C@@H](CC(=O)O)NC(=O)[C@H](CC(C)C)n3cc(CCN4CC(OC)C4)c(C(F)(F)F)nc3=O)c(F)c3c2CCC3)c(C)c1] |
| | 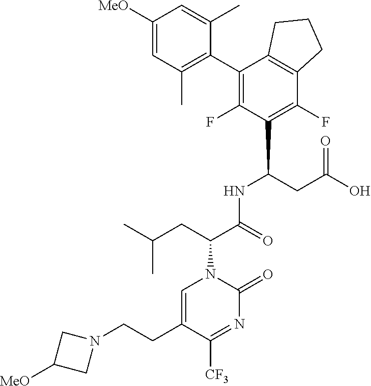 [SMILES: COc1cc(C)c(-c2c(F)c([C@@H](CC(=O)O)NC(=O)[C@@H](CC(C)C)n3cc(CCN4CC(OC)C4)c(C(F)(F)F)nc3=O)c(F)c3c2CCC3)c(C)c1] |
| 292 | 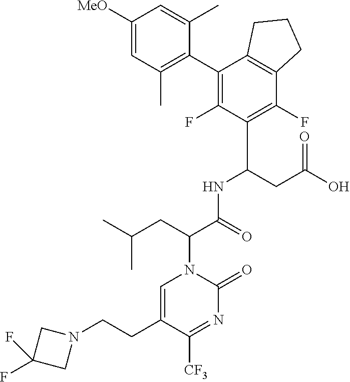 [SMILES: COc1cc(C)c(-c2c(F)c(C(CC(=O)O)NC(=O)C(CC(C)C)n3cc(CCN4CC(F)(F)C4)c(C(F)(F)F)nc3=O)c(F)c3c2CCC3)c(C)c1] |
| | 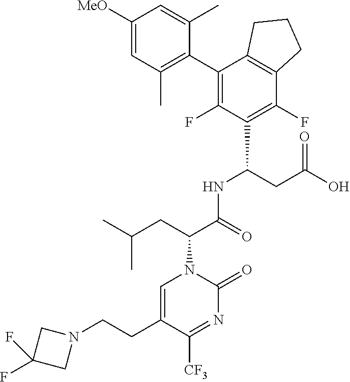 [SMILES: COc1cc(C)c(-c2c(F)c([C@H](CC(=O)O)NC(=O)[C@@H](CC(C)C)n3cc(CCN4CC(F)(F)C4)c(C(F)(F)F)nc3=O)c(F)c3c2CCC3)c(C)c1] |
| | 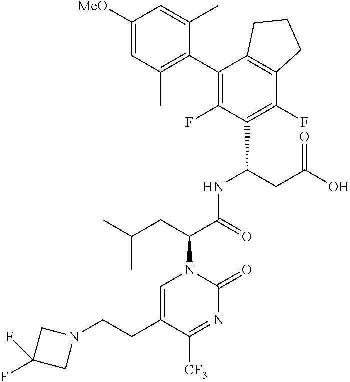 [SMILES: COc1cc(C)c(-c2c(F)c([C@H](CC(=O)O)NC(=O)[C@H](CC(C)C)n3cc(CCN4CC(F)(F)C4)c(C(F)(F)F)nc3=O)c(F)c3c2CCC3)c(C)c1] |
| | 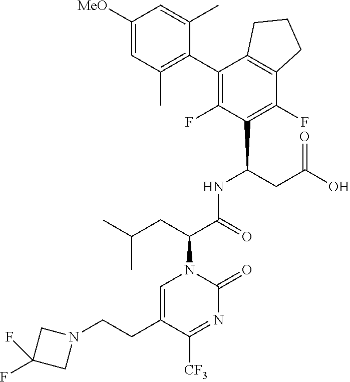 [SMILES: COc1cc(C)c(-c2c(F)c([C@@H](CC(=O)O)NC(=O)[C@H](CC(C)C)n3cc(CCN4CC(F)(F)C4)c(C(F)(F)F)nc3=O)c(F)c3c2CCC3)c(C)c1] |
| | 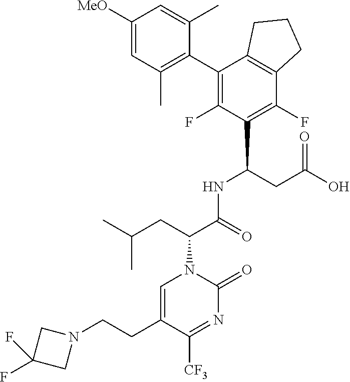 [SMILES: COc1cc(C)c(-c2c(F)c([C@@H](CC(=O)O)NC(=O)[C@@H](CC(C)C)n3cc(CCN4CC(F)(F)C4)c(C(F)(F)F)nc3=O)c(F)c3c2CCC3)c(C)c1] |
| 293 | 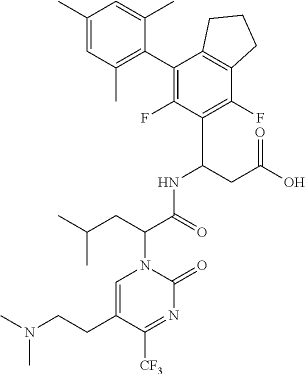 [SMILES: Cc1cc(C)c(-c2c(F)c(C(CC(=O)O)NC(=O)C(CC(C)C)n3cc(CCN(C)C)c(C(F)(F)F)nc3=O)c(F)c3c2CCC3)c(C)c1] |
| | 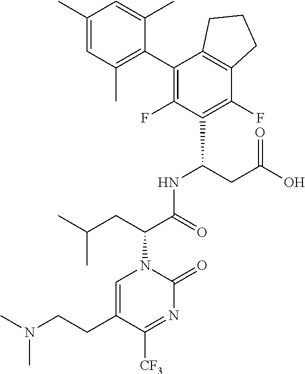 [SMILES: Cc1cc(C)c(-c2c(F)c([C@H](CC(=O)O)NC(=O)[C@@H](CC(C)C)n3cc(CCN(C)C)c(C(F)(F)F)nc3=O)c(F)c3c2CCC3)c(C)c1] |
| | 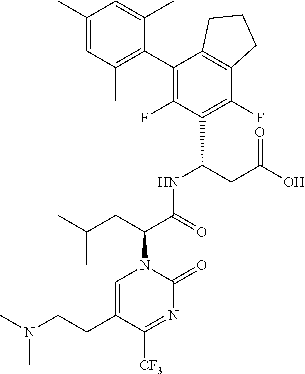 [SMILES: Cc1cc(C)c(-c2c(F)c([C@H](CC(=O)O)NC(=O)[C@H](CC(C)C)n3cc(CCN(C)C)c(C(F)(F)F)nc3=O)c(F)c3c2CCC3)c(C)c1] |
| | 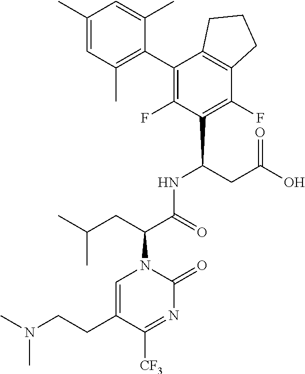 [SMILES: Cc1cc(C)c(-c2c(F)c([C@@H](CC(=O)O)NC(=O)[C@H](CC(C)C)n3cc(CCN(C)C)c(C(F)(F)F)nc3=O)c(F)c3c2CCC3)c(C)c1] |
| | 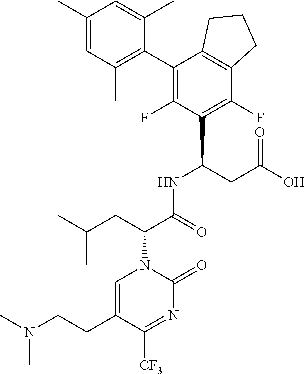 [SMILES: Cc1cc(C)c(-c2c(F)c([C@@H](CC(=O)O)NC(=O)[C@@H](CC(C)C)n3cc(CCN(C)C)c(C(F)(F)F)nc3=O)c(F)c3c2CCC3)c(C)c1] |
| 294 | 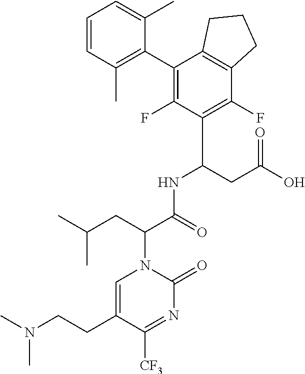 [SMILES: Cc1cccc(C)c1-c1c(F)c(C(CC(=O)O)NC(=O)C(CC(C)C)n2cc(CCN(C)C)c(C(F)(F)F)nc2=O)c(F)c2c1CCC2] |
| | 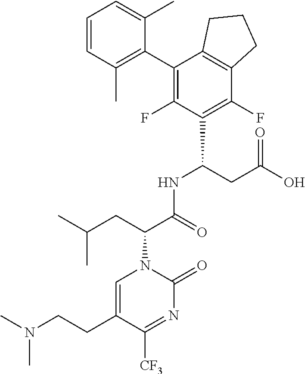 [SMILES: Cc1cccc(C)c1-c1c(F)c([C@H](CC(=O)O)NC(=O)[C@@H](CC(C)C)n2cc(CCN(C)C)c(C(F)(F)F)nc2=O)c(F)c2c1CCC2] |
| | 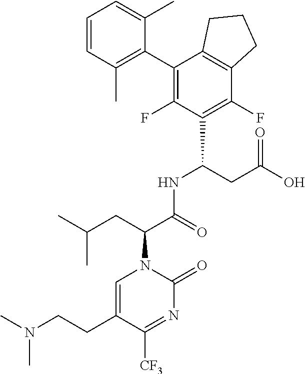 [SMILES: Cc1cccc(C)c1-c1c(F)c([C@H](CC(=O)O)NC(=O)[C@H](CC(C)C)n2cc(CCN(C)C)c(C(F)(F)F)nc2=O)c(F)c2c1CCC2] |
| | 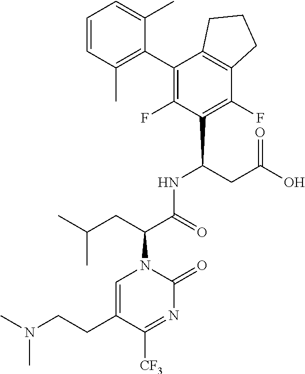 [SMILES: Cc1cccc(C)c1-c1c(F)c([C@@H](CC(=O)O)NC(=O)[C@H](CC(C)C)n2cc(CCN(C)C)c(C(F)(F)F)nc2=O)c(F)c2c1CCC2] |
| | 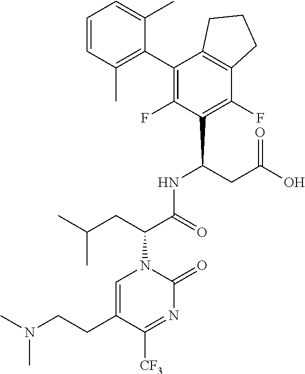 [SMILES: Cc1cccc(C)c1-c1c(F)c([C@@H](CC(=O)O)NC(=O)[C@@H](CC(C)C)n2cc(CCN(C)C)c(C(F)(F)F)nc2=O)c(F)c2c1CCC2] |
| 295 | 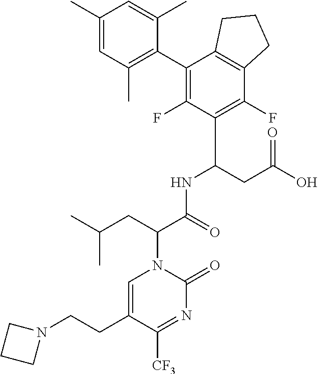 [SMILES: Cc1cc(C)c(-c2c(F)c(C(CC(=O)O)NC(=O)C(CC(C)C)n3cc(CCN4CCC4)c(C(F)(F)F)nc3=O)c(F)c3c2CCC3)c(C)c1] |
| | 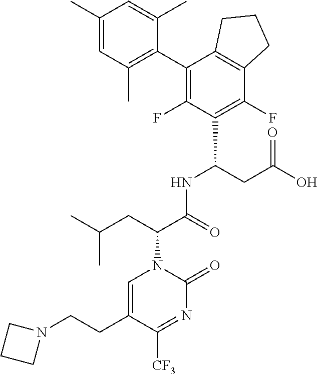 [SMILES: Cc1cc(C)c(-c2c(F)c([C@H](CC(=O)O)NC(=O)[C@@H](CC(C)C)n3cc(CCN4CCC4)c(C(F)(F)F)nc3=O)c(F)c3c2CCC3)c(C)c1] |
| | 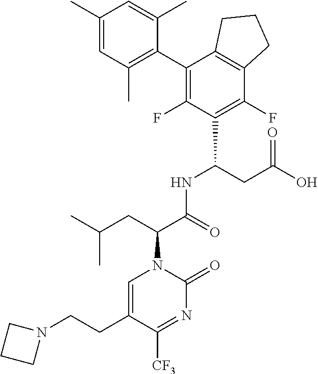 [SMILES: Cc1cc(C)c(-c2c(F)c([C@H](CC(=O)O)NC(=O)[C@H](CC(C)C)n3cc(CCN4CCC4)c(C(F)(F)F)nc3=O)c(F)c3c2CCC3)c(C)c1] |
| | 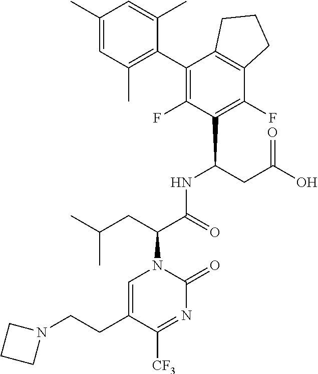 [SMILES: Cc1cc(C)c(-c2c(F)c([C@@H](CC(=O)O)NC(=O)[C@H](CC(C)C)n3cc(CCN4CCC4)c(C(F)(F)F)nc3=O)c(F)c3c2CCC3)c(C)c1] |
| | 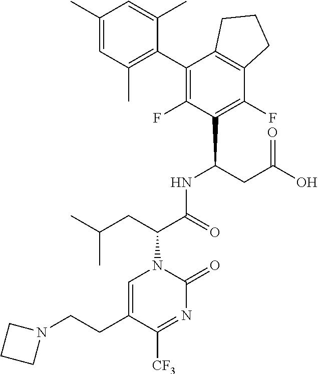 [SMILES: Cc1cc(C)c(-c2c(F)c([C@@H](CC(=O)O)NC(=O)[C@@H](CC(C)C)n3cc(CCN4CCC4)c(C(F)(F)F)nc3=O)c(F)c3c2CCC3)c(C)c1] |
| 296 | 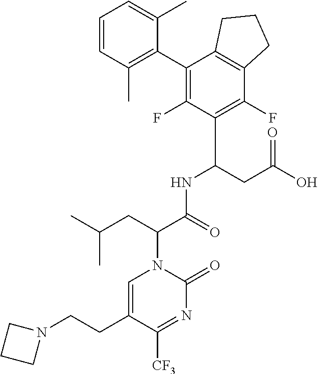 [SMILES: Cc1cccc(C)c1-c1c(F)c(C(CC(=O)O)NC(=O)C(CC(C)C)n2cc(CCN3CCC3)c(C(F)(F)F)nc2=O)c(F)c2c1CCC2] |
| | 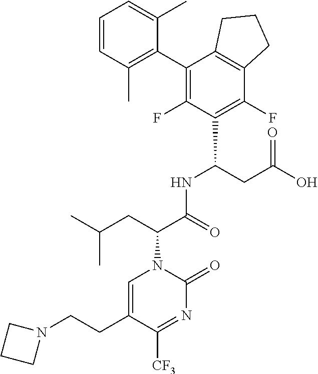 [SMILES: Cc1cccc(C)c1-c1c(F)c([C@H](CC(=O)O)NC(=O)[C@@H](CC(C)C)n2cc(CCN3CCC3)c(C(F)(F)F)nc2=O)c(F)c2c1CCC2] |
| | 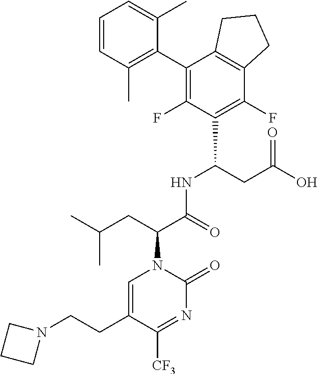 [SMILES: Cc1cccc(C)c1-c1c(F)c([C@H](CC(=O)O)NC(=O)[C@H](CC(C)C)n2cc(CCN3CCC3)c(C(F)(F)F)nc2=O)c(F)c2c1CCC2] |
| | 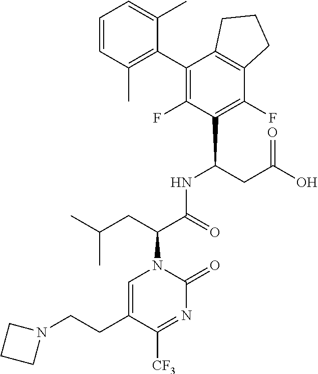 [SMILES: Cc1cccc(C)c1-c1c(F)c([C@@H](CC(=O)O)NC(=O)[C@H](CC(C)C)n2cc(CCN3CCC3)c(C(F)(F)F)nc2=O)c(F)c2c1CCC2] |
| | 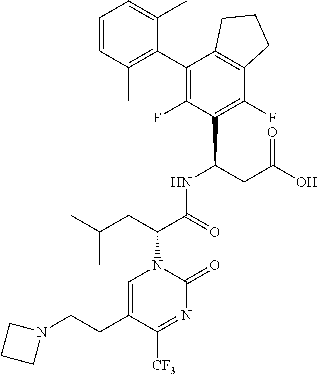 [SMILES: Cc1cccc(C)c1-c1c(F)c([C@@H](CC(=O)O)NC(=O)[C@@H](CC(C)C)n2cc(CCN3CCC3)c(C(F)(F)F)nc2=O)c(F)c2c1CCC2] |
| 297 | 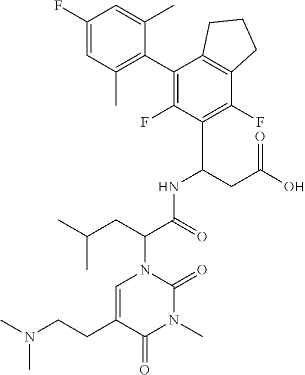 [SMILES: Cc1cc(F)cc(C)c1-c1c(F)c(C(CC(=O)O)NC(=O)C(CC(C)C)n2cc(CCN(C)C)c(=O)n(C)c2=O)c(F)c2c1CCC2] |
| | 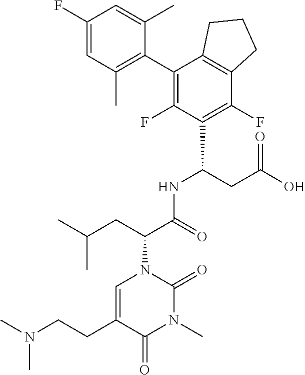 [SMILES: Cc1cc(F)cc(C)c1-c1c(F)c([C@H](CC(=O)O)NC(=O)[C@@H](CC(C)C)n2cc(CCN(C)C)c(=O)n(C)c2=O)c(F)c2c1CCC2] |
| | 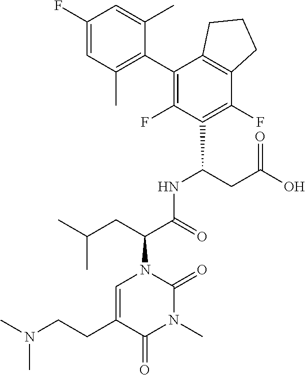 [SMILES: Cc1cc(F)cc(C)c1-c1c(F)c([C@H](CC(=O)O)NC(=O)[C@H](CC(C)C)n2cc(CCN(C)C)c(=O)n(C)c2=O)c(F)c2c1CCC2] |
| | 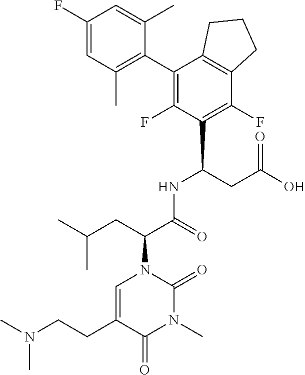 [SMILES: Cc1cc(F)cc(C)c1-c1c(F)c([C@@H](CC(=O)O)NC(=O)[C@H](CC(C)C)n2cc(CCN(C)C)c(=O)n(C)c2=O)c(F)c2c1CCC2] |
| | 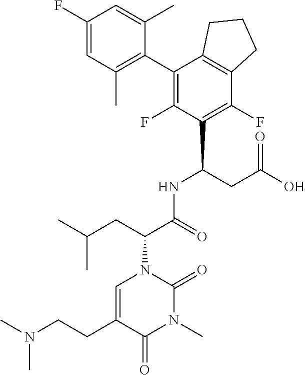 [SMILES: Cc1cc(F)cc(C)c1-c1c(F)c([C@@H](CC(=O)O)NC(=O)[C@@H](CC(C)C)n2cc(CCN(C)C)c(=O)n(C)c2=O)c(F)c2c1CCC2] |
| 298 | 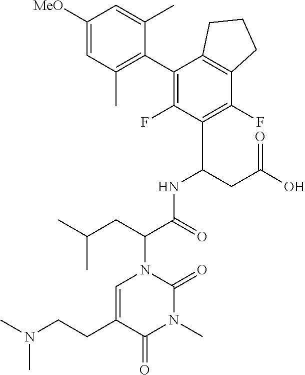 [SMILES: COc1cc(C)c(-c2c(F)c(C(CC(=O)O)NC(=O)C(CC(C)C)n3cc(CCN(C)C)c(=O)n(C)c3=O)c(F)c3c2CCC3)c(C)c1] |
| | 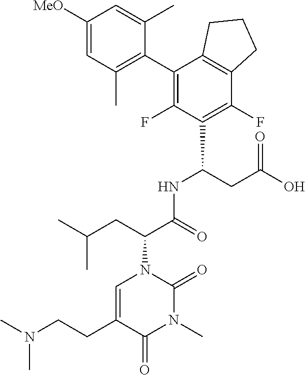 [SMILES: COc1cc(C)c(-c2c(F)c([C@H](CC(=O)O)NC(=O)[C@@H](CC(C)C)n3cc(CCN(C)C)c(=O)n(C)c3=O)c(F)c3c2CCC3)c(C)c1] |
| | 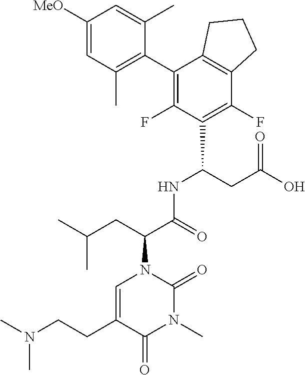 [SMILES: COc1cc(C)c(-c2c(F)c([C@H](CC(=O)O)NC(=O)[C@H](CC(C)C)n3cc(CCN(C)C)c(=O)n(C)c3=O)c(F)c3c2CCC3)c(C)c1] |
| | 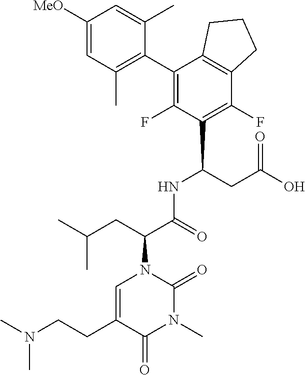 [SMILES: COc1cc(C)c(-c2c(F)c([C@@H](CC(=O)O)NC(=O)[C@H](CC(C)C)n3cc(CCN(C)C)c(=O)n(C)c3=O)c(F)c3c2CCC3)c(C)c1] |
| | 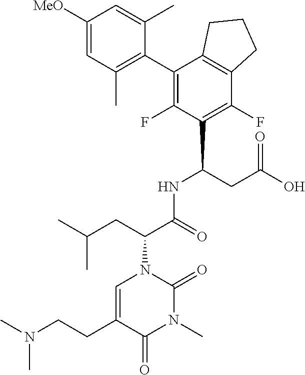 [SMILES: COc1cc(C)c(-c2c(F)c([C@@H](CC(=O)O)NC(=O)[C@@H](CC(C)C)n3cc(CCN(C)C)c(=O)n(C)c3=O)c(F)c3c2CCC3)c(C)c1] |
| 299 | 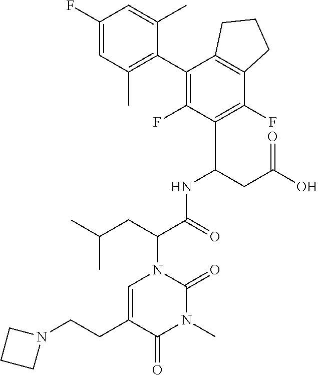 [SMILES: Cc1cc(F)cc(C)c1-c1c(F)c(C(CC(=O)O)NC(=O)C(CC(C)C)n2cc(CCN3CCC3)c(=O)n(C)c2=O)c(F)c2c1CCC2] |
| | 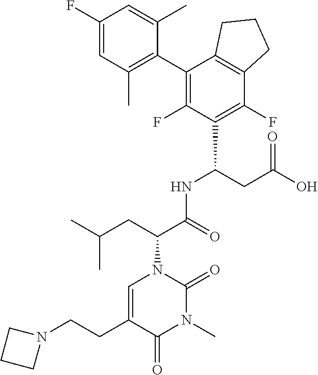 [SMILES: Cc1cc(F)cc(C)c1-c1c(F)c([C@H](CC(=O)O)NC(=O)[C@@H](CC(C)C)n2cc(CCN3CCC3)c(=O)n(C)c2=O)c(F)c2c1CCC2] |
| | 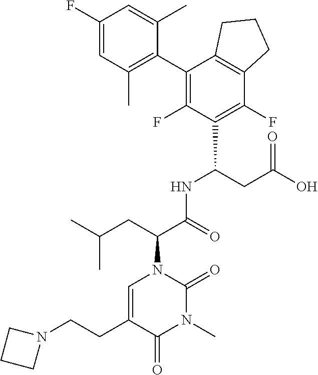 [SMILES: Cc1cc(F)cc(C)c1-c1c(F)c([C@H](CC(=O)O)NC(=O)[C@H](CC(C)C)n2cc(CCN3CCC3)c(=O)n(C)c2=O)c(F)c2c1CCC2] |
| | 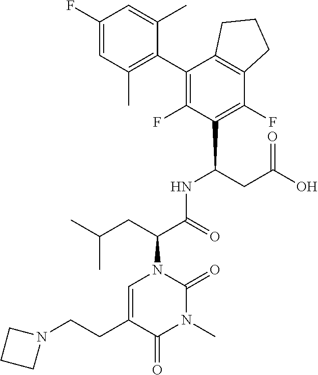 [SMILES: Cc1cc(F)cc(C)c1-c1c(F)c([C@@H](CC(=O)O)NC(=O)[C@H](CC(C)C)n2cc(CCN3CCC3)c(=O)n(C)c2=O)c(F)c2c1CCC2] |
| | 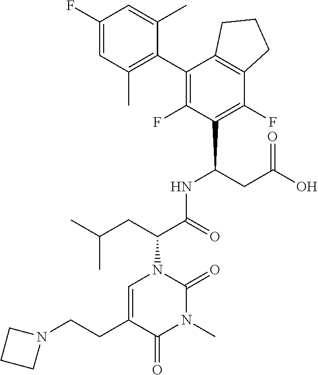 [SMILES: Cc1cc(F)cc(C)c1-c1c(F)c([C@@H](CC(=O)O)NC(=O)[C@@H](CC(C)C)n2cc(CCN3CCC3)c(=O)n(C)c2=O)c(F)c2c1CCC2] |
| 300 | 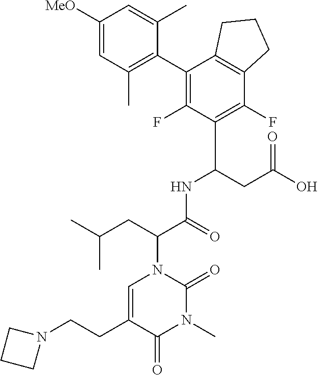 [SMILES: COc1cc(C)c(-c2c(F)c(C(CC(=O)O)NC(=O)C(CC(C)C)n3cc(CCN4CCC4)c(=O)n(C)c3=O)c(F)c3c2CCC3)c(C)c1] |
| | 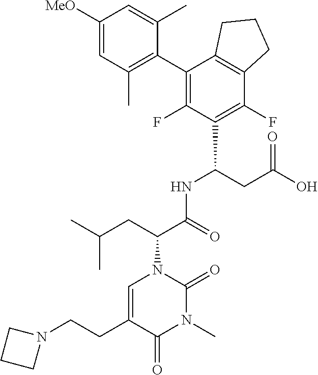 [SMILES: COc1cc(C)c(-c2c(F)c([C@H](CC(=O)O)NC(=O)[C@@H](CC(C)C)n3cc(CCN4CCC4)c(=O)n(C)c3=O)c(F)c3c2CCC3)c(C)c1] |
| | 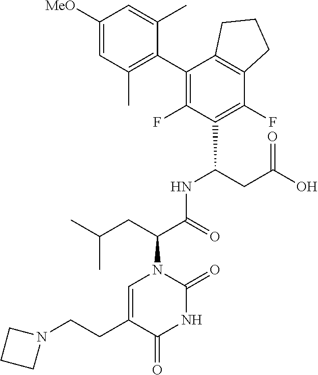 [SMILES: COc1cc(C)c(-c2c(F)c([C@H](CC(=O)O)NC(=O)[C@H](CC(C)C)n3cc(CCN4CCC4)c(=O)[nH]c3=O)c(F)c3c2CCC3)c(C)c1] |
| | 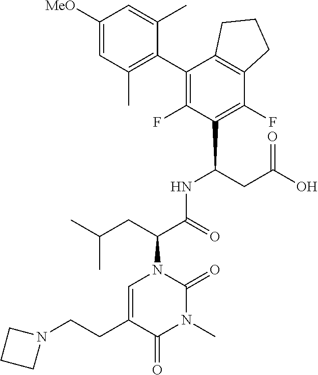 [SMILES: COc1cc(C)c(-c2c(F)c([C@@H](CC(=O)O)NC(=O)[C@H](CC(C)C)n3cc(CCN4CCC4)c(=O)n(C)c3=O)c(F)c3c2CCC3)c(C)c1] |
| | 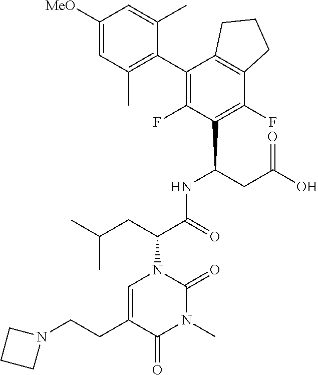 [SMILES: COc1cc(C)c(-c2c(F)c([C@@H](CC(=O)O)NC(=O)[C@@H](CC(C)C)n3cc(CCN4CCC4)c(=O)n(C)c3=O)c(F)c3c2CCC3)c(C)c1] |
| 301 | 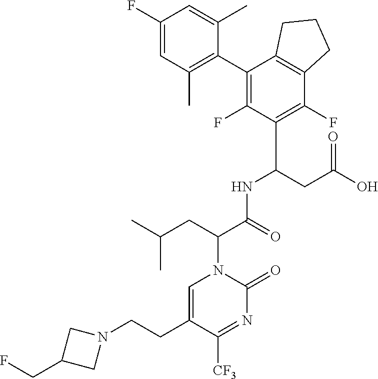 [SMILES: Cc1cc(F)cc(C)c1-c1c(F)c(C(CC(=O)O)NC(=O)C(CC(C)C)n2cc(CCN3CC(CF)C3)c(C(F)(F)F)nc2=O)c(F)c2c1CCC2] |
| | 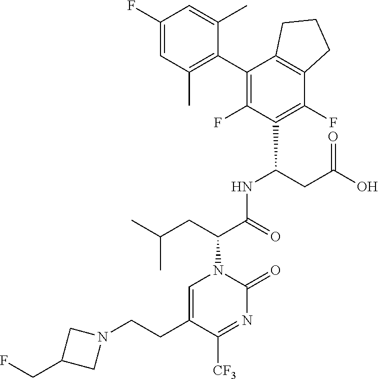 [SMILES: Cc1cc(F)cc(C)c1-c1c(F)c([C@H](CC(=O)O)NC(=O)[C@@H](CC(C)C)n2cc(CCN3CC(CF)C3)c(C(F)(F)F)nc2=O)c(F)c2c1CCC2] |
| | 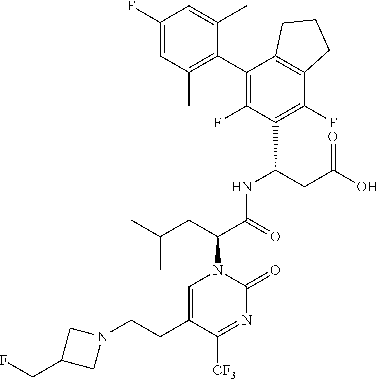 [SMILES: Cc1cc(F)cc(C)c1-c1c(F)c([C@H](CC(=O)O)NC(=O)[C@H](CC(C)C)n2cc(CCN3CC(CF)C3)c(C(F)(F)F)nc2=O)c(F)c2c1CCC2] |
| | 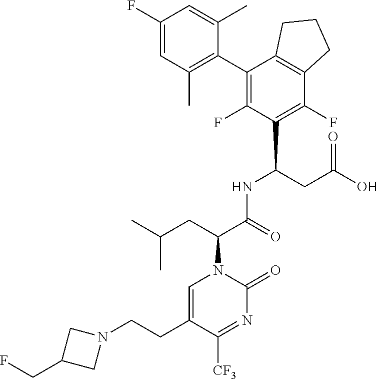 [SMILES: Cc1cc(F)cc(C)c1-c1c(F)c([C@@H](CC(=O)O)NC(=O)[C@H](CC(C)C)n2cc(CCN3CC(CF)C3)c(C(F)(F)F)nc2=O)c(F)c2c1CCC2] |
| | 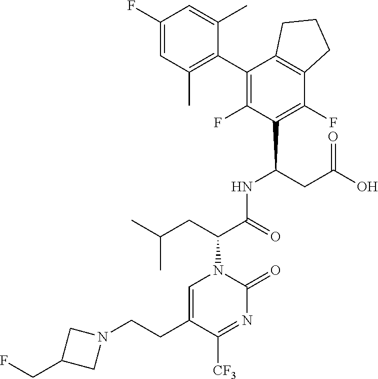 [SMILES: Cc1cc(F)cc(C)c1-c1c(F)c([C@@H](CC(=O)O)NC(=O)[C@@H](CC(C)C)n2cc(CCN3CC(CF)C3)c(C(F)(F)F)nc2=O)c(F)c2c1CCC2] |
| 302 | 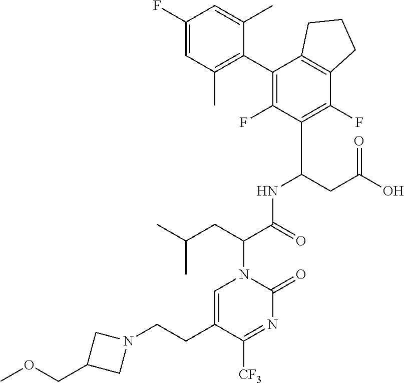 [SMILES: COCC1CN(CCc2cn(C(CC(C)C)C(=O)NC(CC(=O)O)c3c(F)c4c(c(-c5c(C)cc(F)cc5C)c3F)CCC4)c(=O)nc2C(F)(F)F)C1] |
| | 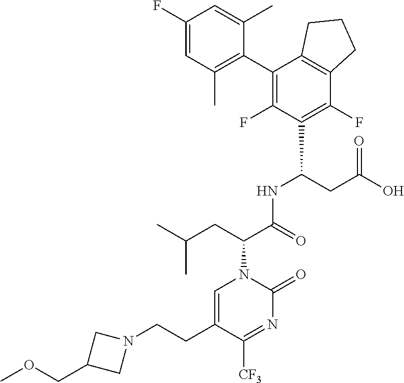 [SMILES: COCC1CN(CCc2cn([C@H](CC(C)C)C(=O)N[C@@H](CC(=O)O)c3c(F)c4c(c(-c5c(C)cc(F)cc5C)c3F)CCC4)c(=O)nc2C(F)(F)F)C1] |
| | 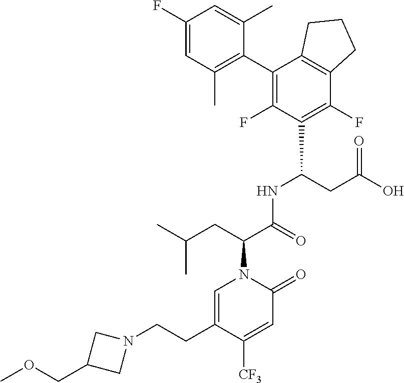 [SMILES: COCC1CN(CCc2cn([C@@H](CC(C)C)C(=O)N[C@@H](CC(=O)O)c3c(F)c4c(c(-c5c(C)cc(F)cc5C)c3F)CCC4)c(=O)cc2C(F)(F)F)C1] |
| | 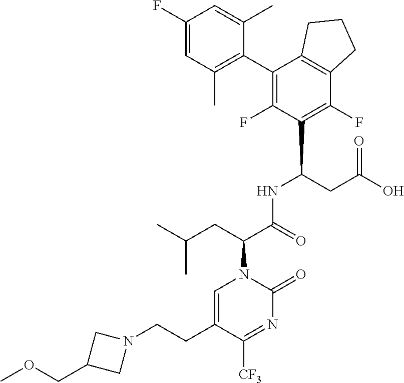 [SMILES: COCC1CN(CCc2cn([C@@H](CC(C)C)C(=O)N[C@H](CC(=O)O)c3c(F)c4c(c(-c5c(C)cc(F)cc5C)c3F)CCC4)c(=O)nc2C(F)(F)F)C1] |
| | 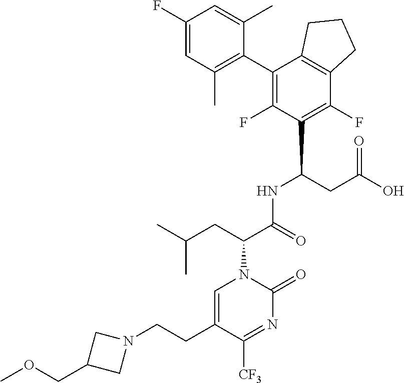 [SMILES: COCC1CN(CCc2cn([C@H](CC(C)C)C(=O)N[C@H](CC(=O)O)c3c(F)c4c(c(-c5c(C)cc(F)cc5C)c3F)CCC4)c(=O)nc2C(F)(F)F)C1] |
| 303 | 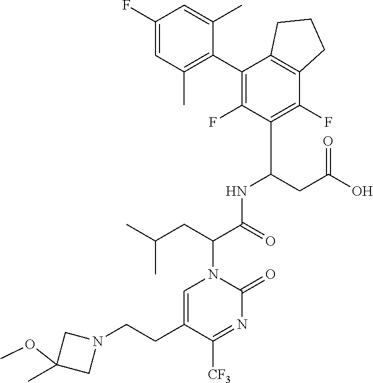 [SMILES: COC1(C)CN(CCc2cn(C(CC(C)C)C(=O)NC(CC(=O)O)c3c(F)c4c(c(-c5c(C)cc(F)cc5C)c3F)CCC4)c(=O)nc2C(F)(F)F)C1] |
| | 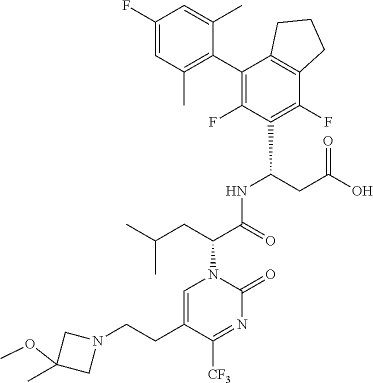 [SMILES: COC1(C)CN(CCc2cn([C@H](CC(C)C)C(=O)N[C@@H](CC(=O)O)c3c(F)c4c(c(-c5c(C)cc(F)cc5C)c3F)CCC4)c(=O)nc2C(F)(F)F)C1] |
| | 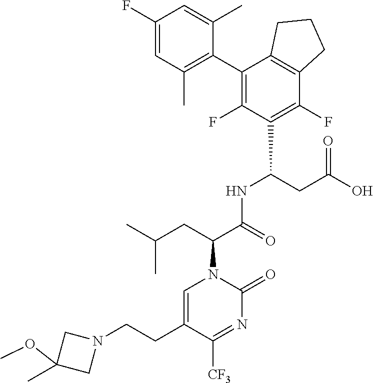 [SMILES: COC1(C)CN(CCc2cn([C@@H](CC(C)C)C(=O)N[C@@H](CC(=O)O)c3c(F)c4c(c(-c5c(C)cc(F)cc5C)c3F)CCC4)c(=O)nc2C(F)(F)F)C1] |
| | 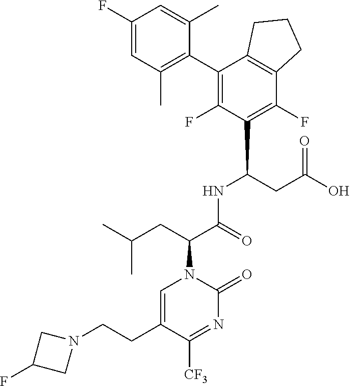 [SMILES: Cc1cc(F)cc(C)c1-c1c(F)c([C@@H](CC(=O)O)NC(=O)[C@H](CC(C)C)n2cc(CCN3CC(F)C3)c(C(F)(F)F)nc2=O)c(F)c2c1CCC2] |
| | 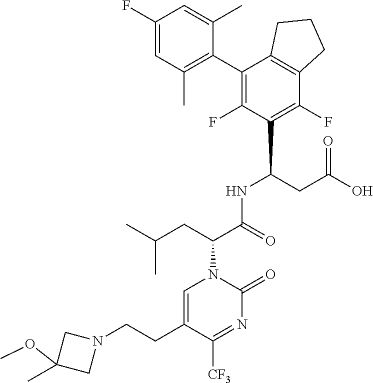 [SMILES: COC1(C)CN(CCc2cn([C@H](CC(C)C)C(=O)N[C@H](CC(=O)O)c3c(F)c4c(c(-c5c(C)cc(F)cc5C)c3F)CCC4)c(=O)nc2C(F)(F)F)C1] |
| 304 | 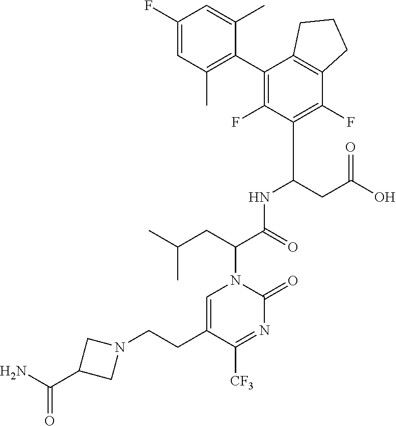 [SMILES: Cc1cc(F)cc(C)c1-c1c(F)c(C(CC(=O)O)NC(=O)C(CC(C)C)n2cc(CCN3CC(C(N)=O)C3)c(C(F)(F)F)nc2=O)c(F)c2c1CCC2] |
| | 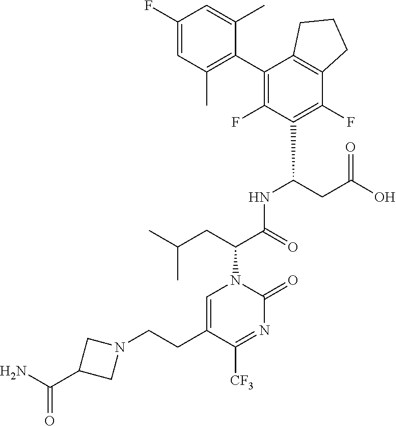 [SMILES: Cc1cc(F)cc(C)c1-c1c(F)c([C@H](CC(=O)O)NC(=O)[C@@H](CC(C)C)n2cc(CCN3CC(C(N)=O)C3)c(C(F)(F)F)nc2=O)c(F)c2c1CCC2] |
| | 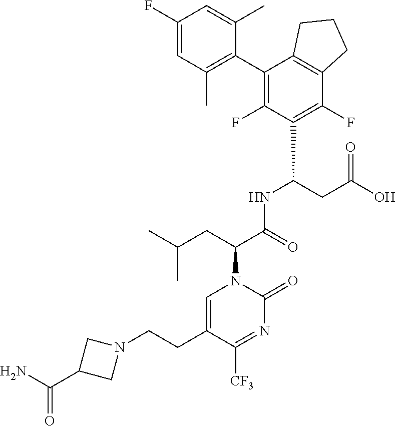 [SMILES: Cc1cc(F)cc(C)c1-c1c(F)c([C@H](CC(=O)O)NC(=O)[C@H](CC(C)C)n2cc(CCN3CC(C(N)=O)C3)c(C(F)(F)F)nc2=O)c(F)c2c1CCC2] |
| | 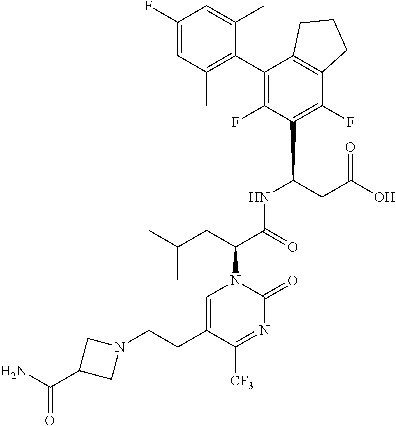 [SMILES: Cc1cc(F)cc(C)c1-c1c(F)c([C@@H](CC(=O)O)NC(=O)[C@H](CC(C)C)n2cc(CCN3CC(C(N)=O)C3)c(C(F)(F)F)nc2=O)c(F)c2c1CCC2] |
| | 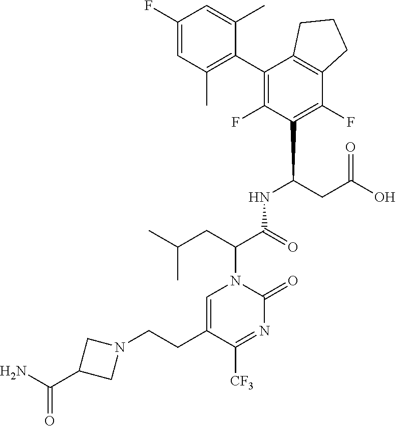 |
| 305 | 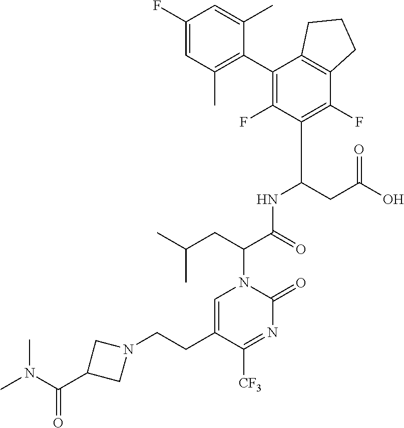 [SMILES: Cc1cc(F)cc(C)c1-c1c(F)c(C(CC(=O)O)NC(=O)C(CC(C)C)n2cc(CCN3CC(C(=O)N(C)C)C3)c(C(F)(F)F)nc2=O)c(F)c2c1CCC2] |
| | 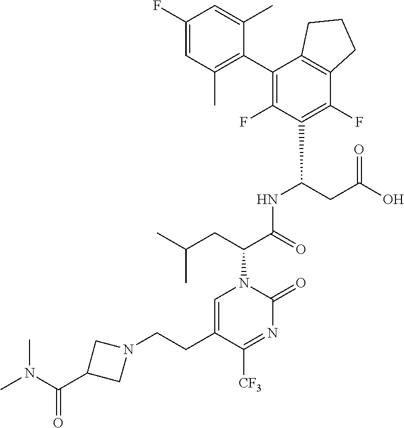 [SMILES: Cc1cc(F)cc(C)c1-c1c(F)c([C@H](CC(=O)O)NC(=O)[C@@H](CC(C)C)n2cc(CCN3CC(C(=O)N(C)C)C3)c(C(F)(F)F)nc2=O)c(F)c2c1CCC2] |
| | 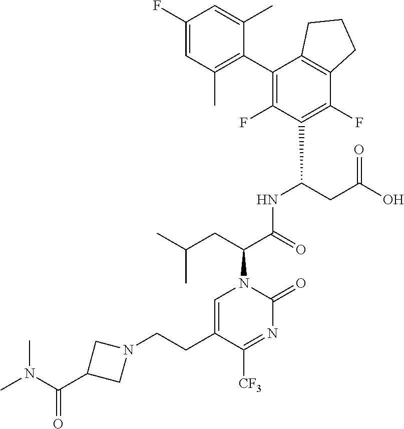 [SMILES: Cc1cc(F)cc(C)c1-c1c(F)c([C@H](CC(=O)O)NC(=O)[C@H](CC(C)C)n2cc(CCN3CC(C(=O)N(C)C)C3)c(C(F)(F)F)nc2=O)c(F)c2c1CCC2] |
| | 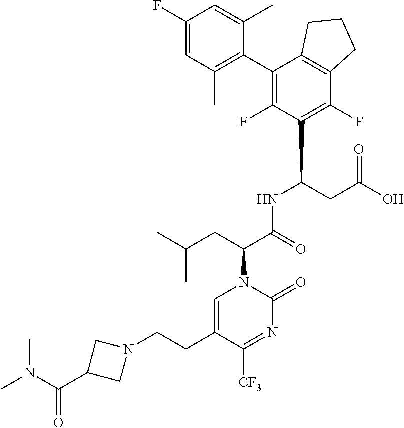 [SMILES: Cc1cc(F)cc(C)c1-c1c(F)c([C@@H](CC(=O)O)NC(=O)[C@H](CC(C)C)n2cc(CCN3CC(C(=O)N(C)C)C3)c(C(F)(F)F)nc2=O)c(F)c2c1CCC2] |
| | 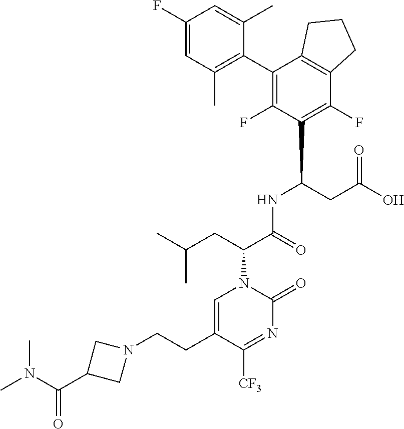 [SMILES: Cc1cc(F)cc(C)c1-c1c(F)c([C@@H](CC(=O)O)NC(=O)[C@@H](CC(C)C)n2cc(CCN3CC(C(=O)N(C)C)C3)c(C(F)(F)F)nc2=O)c(F)c2c1CCC2] |
| 307 | 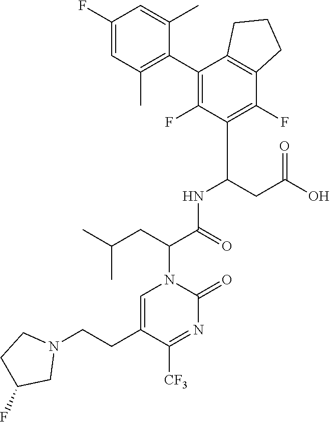 [SMILES: Cc1cc(F)cc(C)c1-c1c(F)c(C(CC(=O)O)NC(=O)C(CC(C)C)n2cc(CCN3CC[C@@H](F)C3)c(C(F)(F)F)nc2=O)c(F)c2c1CCC2] |
| | 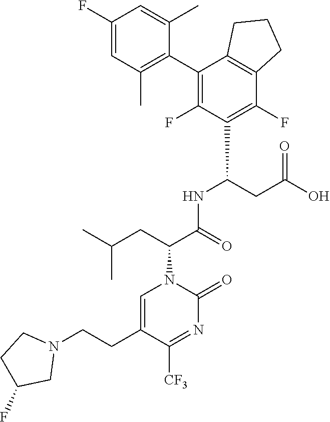 [SMILES: Cc1cc(F)cc(C)c1-c1c(F)c([C@H](CC(=O)O)NC(=O)[C@@H](CC(C)C)n2cc(CCN3CC[C@@H](F)C3)c(C(F)(F)F)nc2=O)c(F)c2c1CCC2] |
| | 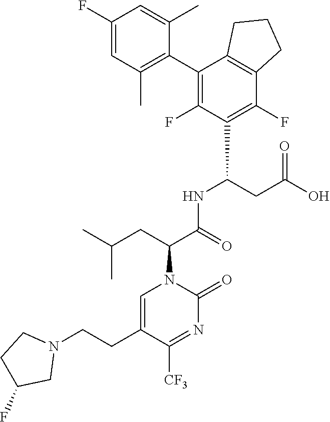 [SMILES: Cc1cc(F)cc(C)c1-c1c(F)c([C@H](CC(=O)O)NC(=O)[C@H](CC(C)C)n2cc(CCN3CC[C@@H](F)C3)c(C(F)(F)F)nc2=O)c(F)c2c1CCC2] |
| | 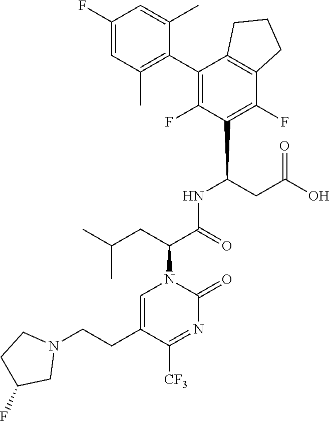 [SMILES: Cc1cc(F)cc(C)c1-c1c(F)c([C@@H](CC(=O)O)NC(=O)[C@H](CC(C)C)n2cc(CCN3CC[C@@H](F)C3)c(C(F)(F)F)nc2=O)c(F)c2c1CCC2] |
| | 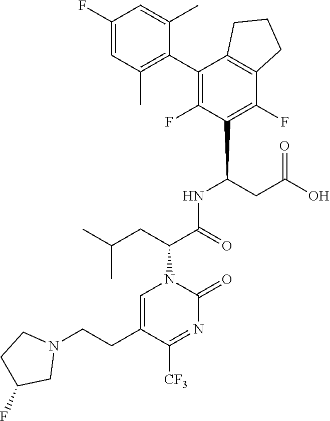 [SMILES: Cc1cc(F)cc(C)c1-c1c(F)c([C@@H](CC(=O)O)NC(=O)[C@@H](CC(C)C)n2cc(CCN3CC[C@@H](F)C3)c(C(F)(F)F)nc2=O)c(F)c2c1CCC2] |
| 307 | 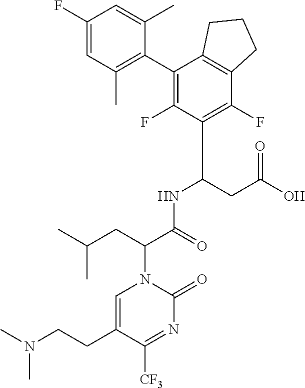 [SMILES: Cc1cc(F)cc(C)c1-c1c(F)c(C(CC(=O)O)NC(=O)C(CC(C)C)n2cc(CCN(C)C)c(C(F)(F)F)nc2=O)c(F)c2c1CCC2] |
| | 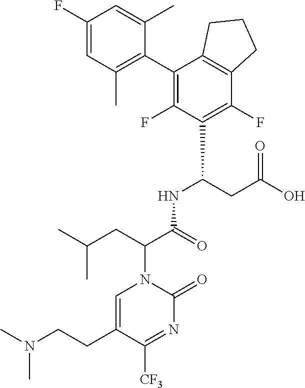 |
| | 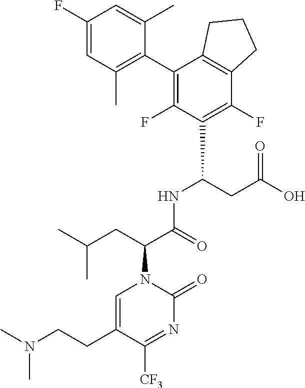 [SMILES: Cc1cc(F)cc(C)c1-c1c(F)c([C@H](CC(=O)O)NC(=O)[C@H](CC(C)C)n2cc(CCN(C)C)c(C(F)(F)F)nc2=O)c(F)c2c1CCC2] |
| | 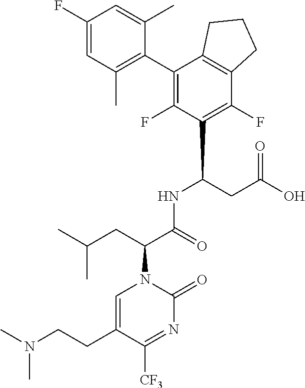 [SMILES: Cc1cc(F)cc(C)c1-c1c(F)c([C@@H](CC(=O)O)NC(=O)[C@H](CC(C)C)n2cc(CCN(C)C)c(C(F)(F)F)nc2=O)c(F)c2c1CCC2] |
| | 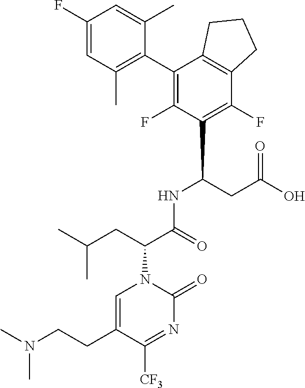 [SMILES: Cc1cc(F)cc(C)c1-c1c(F)c([C@@H](CC(=O)O)NC(=O)[C@@H](CC(C)C)n2cc(CCN(C)C)c(C(F)(F)F)nc2=O)c(F)c2c1CCC2] |
| 308 | 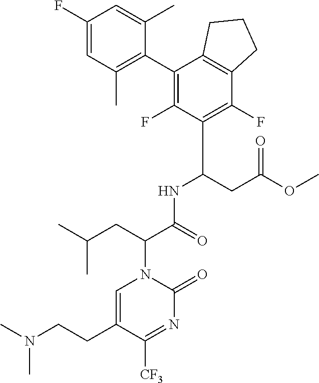 |
| | 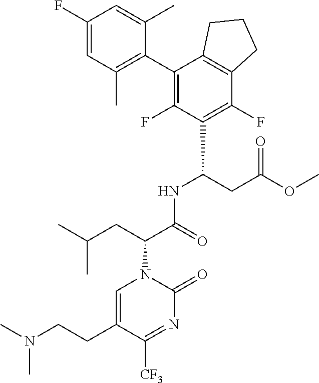 |
| | 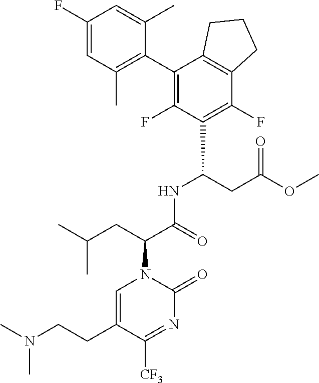 |
| | 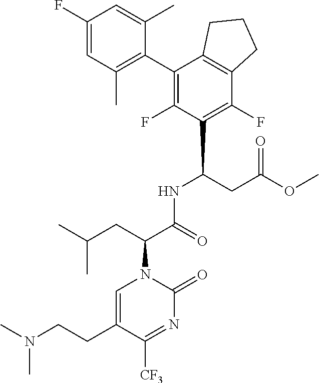 |
| | 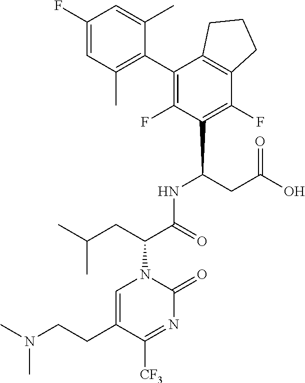 [SMILES: Cc1cc(F)cc(C)c1-c1c(F)c([C@@H](CC(=O)O)NC(=O)[C@@H](CC(C)C)n2cc(CCN(C)C)c(C(F)(F)F)nc2=O)c(F)c2c1CCC2] |
| 309 | 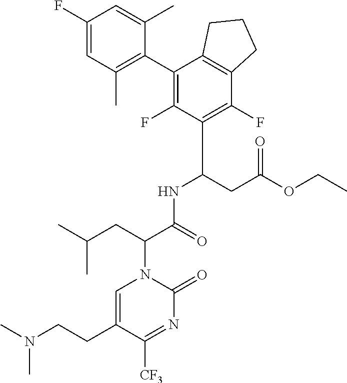 [SMILES: CCOC(=O)CC(NC(=O)C(CC(C)C)n1cc(CCN(C)C)c(C(F)(F)F)nc1=O)c1c(F)c2c(c(-c3c(C)cc(F)cc3C)c1F)CCC2] |
| | 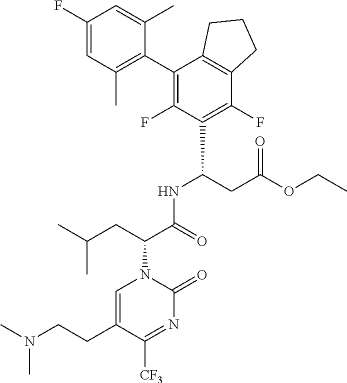 [SMILES: CCOC(=O)C[C@H](NC(=O)[C@@H](CC(C)C)n1cc(CCN(C)C)c(C(F)(F)F)nc1=O)c1c(F)c2c(c(-c3c(C)cc(F)cc3C)c1F)CCC2] |
| | 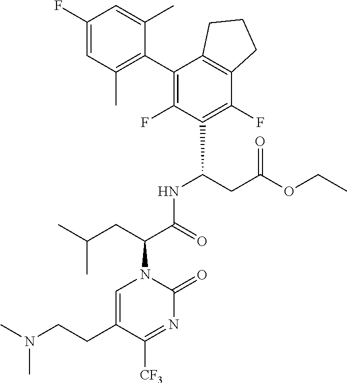 [SMILES: CCOC(=O)C[C@H](NC(=O)[C@H](CC(C)C)n1cc(CCN(C)C)c(C(F)(F)F)nc1=O)c1c(F)c2c(c(-c3c(C)cc(F)cc3C)c1F)CCC2] |
| | 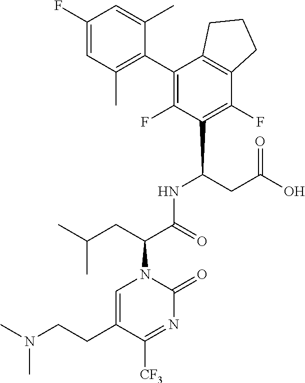 [SMILES: Cc1cc(F)cc(C)c1-c1c(F)c([C@@H](CC(=O)O)NC(=O)[C@H](CC(C)C)n2cc(CCN(C)C)c(C(F)(F)F)nc2=O)c(F)c2c1CCC2] |
| | 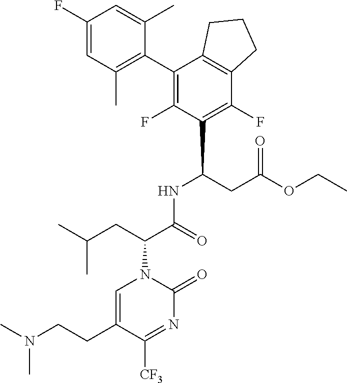 [SMILES: CCOC(=O)C[C@@H](NC(=O)[C@@H](CC(C)C)n1cc(CCN(C)C)c(C(F)(F)F)nc1=O)c1c(F)c2c(c(-c3c(C)cc(F)cc3C)c1F)CCC2] |
| 310 | 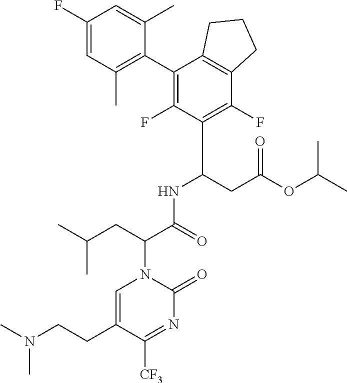 [SMILES: Cc1cc(F)cc(C)c1-c1c(F)c(C(CC(=O)OC(C)C)NC(=O)C(CC(C)C)n2cc(CCN(C)C)c(C(F)(F)F)nc2=O)c(F)c2c1CCC2] |
| | 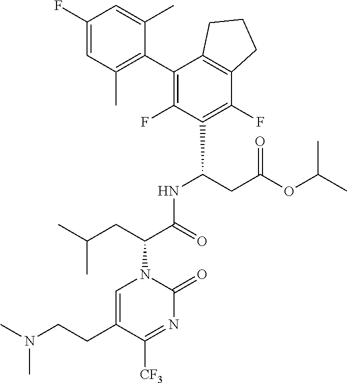 [SMILES: Cc1cc(F)cc(C)c1-c1c(F)c([C@H](CC(=O)OC(C)C)NC(=O)[C@@H](CC(C)C)n2cc(CCN(C)C)c(C(F)(F)F)nc2=O)c(F)c2c1CCC2] |
| | 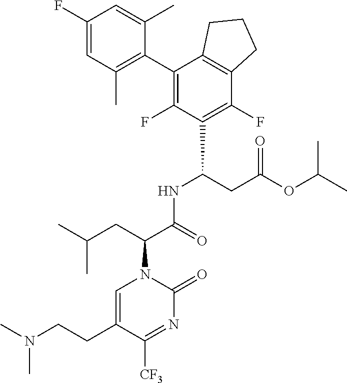 [SMILES: Cc1cc(F)cc(C)c1-c1c(F)c([C@H](CC(=O)OC(C)C)NC(=O)[C@H](CC(C)C)n2cc(CCN(C)C)c(C(F)(F)F)nc2=O)c(F)c2c1CCC2] |
| | 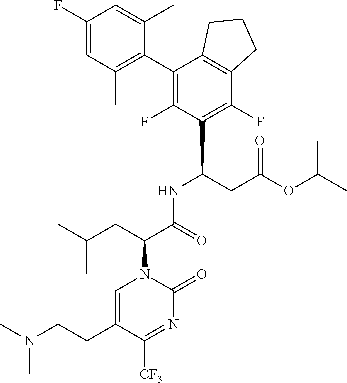 [SMILES: Cc1cc(F)cc(C)c1-c1c(F)c([C@@H](CC(=O)OC(C)C)NC(=O)[C@H](CC(C)C)n2cc(CCN(C)C)c(C(F)(F)F)nc2=O)c(F)c2c1CCC2] |
| | 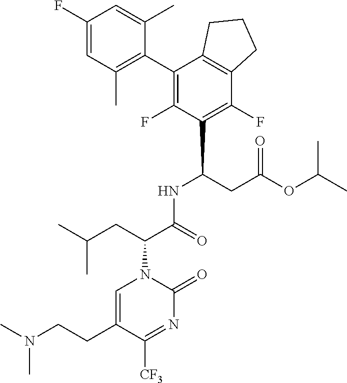 [SMILES: Cc1cc(F)cc(C)c1-c1c(F)c([C@@H](CC(=O)OC(C)C)NC(=O)[C@@H](CC(C)C)n2cc(CCN(C)C)c(C(F)(F)F)nc2=O)c(F)c2c1CCC2] |
| 311 | 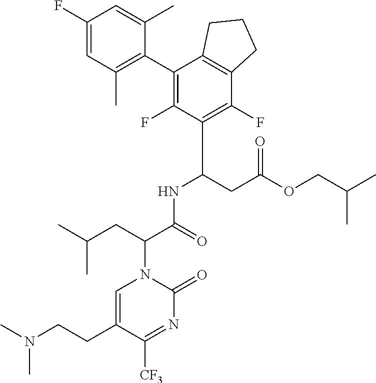 [SMILES: Cc1cc(F)cc(C)c1-c1c(F)c(C(CC(=O)OCC(C)C)NC(=O)C(CC(C)C)n2cc(CCN(C)C)c(C(F)(F)F)nc2=O)c(F)c2c1CCC2] |
| | 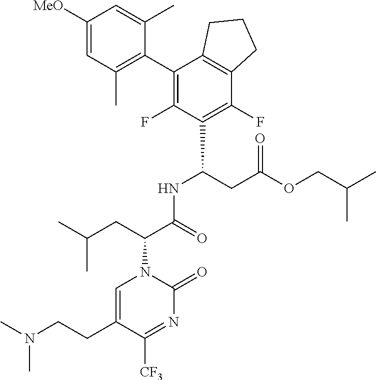 [SMILES: COc1cc(C)c(-c2c(F)c([C@H](CC(=O)OCC(C)C)NC(=O)[C@@H](CC(C)C)n3cc(CCN(C)C)c(C(F)(F)F)nc3=O)c(F)c3c2CCC3)c(C)c1] |
| | 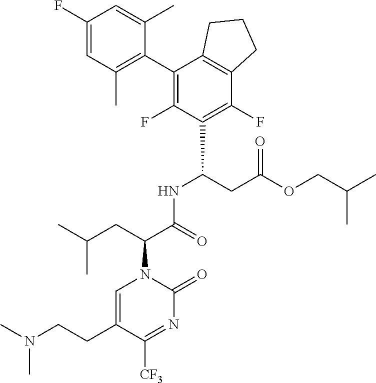 [SMILES: Cc1cc(F)cc(C)c1-c1c(F)c([C@H](CC(=O)OCC(C)C)NC(=O)[C@H](CC(C)C)n2cc(CCN(C)C)c(C(F)(F)F)nc2=O)c(F)c2c1CCC2] |
| | 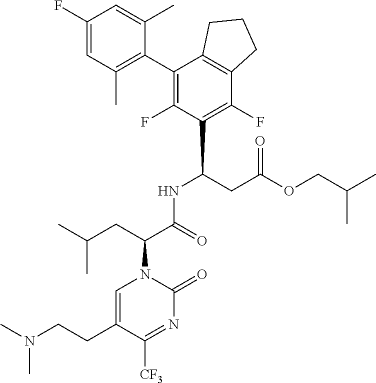 [SMILES: Cc1cc(F)cc(C)c1-c1c(F)c([C@@H](CC(=O)OCC(C)C)NC(=O)[C@H](CC(C)C)n2cc(CCN(C)C)c(C(F)(F)F)nc2=O)c(F)c2c1CCC2] |
| | 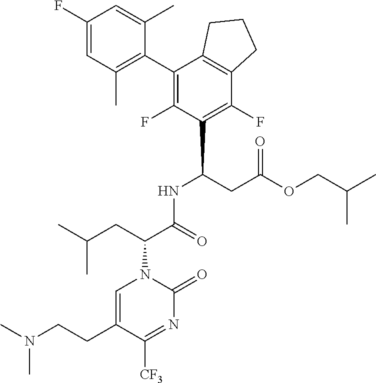 [SMILES: Cc1cc(F)cc(C)c1-c1c(F)c([C@@H](CC(=O)OCC(C)C)NC(=O)[C@@H](CC(C)C)n2cc(CCN(C)C)c(C(F)(F)F)nc2=O)c(F)c2c1CCC2] |
| 312 | 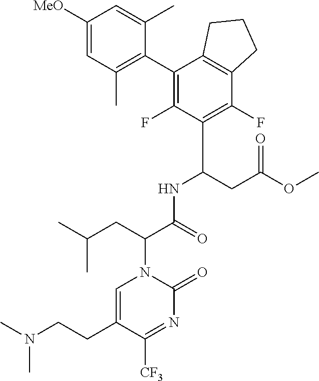 |
| | 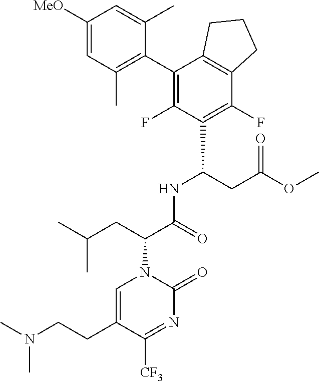 |
| | 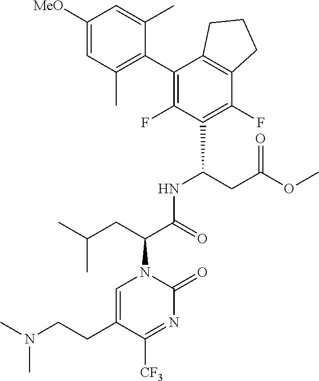 |
| | 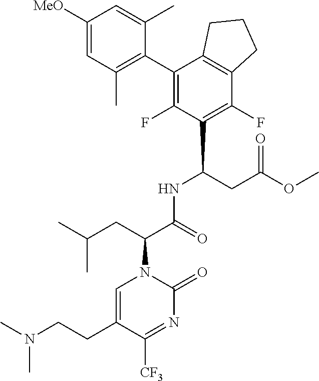 |
| | 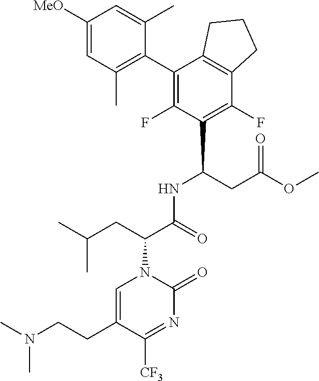 |
| 313 | 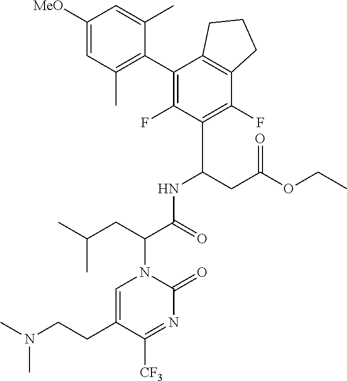 [SMILES: CCOC(=O)CC(NC(=O)C(CC(C)C)n1cc(CCN(C)C)c(C(F)(F)F)nc1=O)c1c(F)c2c(c(-c3c(C)cc(OC)cc3C)c1F)CCC2] |
| | 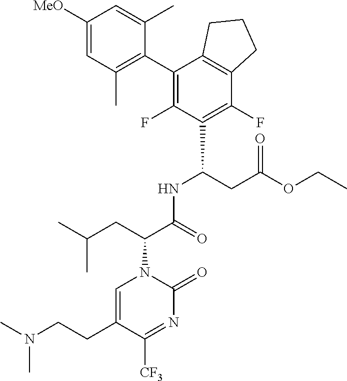 [SMILES: CCOC(=O)C[C@H](NC(=O)[C@@H](CC(C)C)n1cc(CCN(C)C)c(C(F)(F)F)nc1=O)c1c(F)c2c(c(-c3c(C)cc(OC)cc3C)c1F)CCC2] |
| | 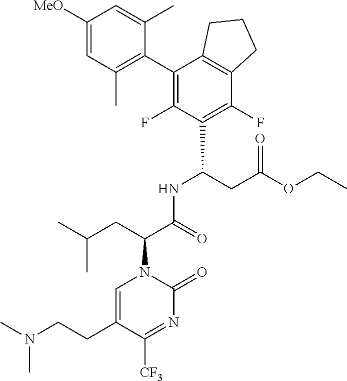 [SMILES: CCOC(=O)C[C@H](NC(=O)[C@H](CC(C)C)n1cc(CCN(C)C)c(C(F)(F)F)nc1=O)c1c(F)c2c(c(-c3c(C)cc(OC)cc3C)c1F)CCC2] |
| | 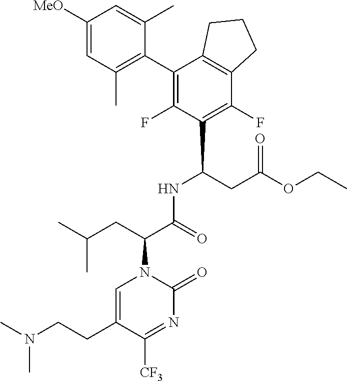 [SMILES: CCOC(=O)C[C@@H](NC(=O)[C@H](CC(C)C)n1cc(CCN(C)C)c(C(F)(F)F)nc1=O)c1c(F)c2c(c(-c3c(C)cc(OC)cc3C)c1F)CCC2] |
| | 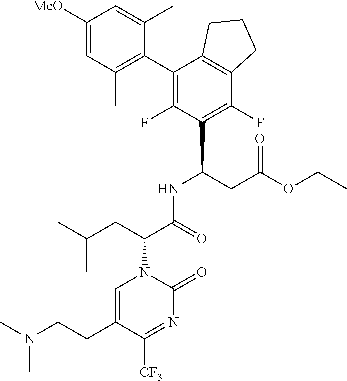 [SMILES: CCOC(=O)C[C@@H](NC(=O)[C@@H](CC(C)C)n1cc(CCN(C)C)c(C(F)(F)F)nc1=O)c1c(F)c2c(c(-c3c(C)cc(OC)cc3C)c1F)CCC2] |
| 314 | 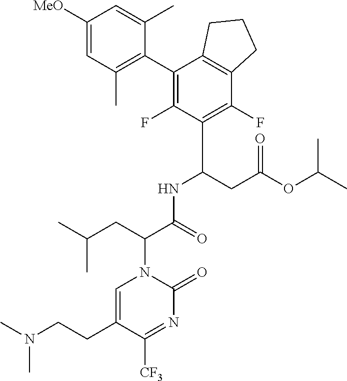 [SMILES: COc1cc(C)c(-c2c(F)c(C(CC(=O)OC(C)C)NC(=O)C(CC(C)C)n3cc(CCN(C)C)c(C(F)(F)F)nc3=O)c(F)c3c2CCC3)c(C)c1] |
| | 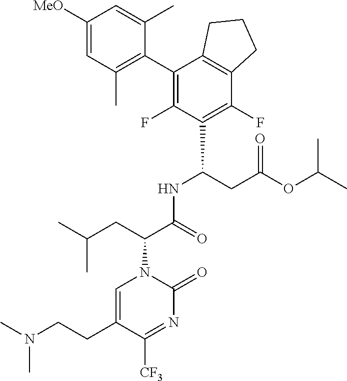 [SMILES: COc1cc(C)c(-c2c(F)c([C@H](CC(=O)OC(C)C)NC(=O)[C@@H](CC(C)C)n3cc(CCN(C)C)c(C(F)(F)F)nc3=O)c(F)c3c2CCC3)c(C)c1] |
| | 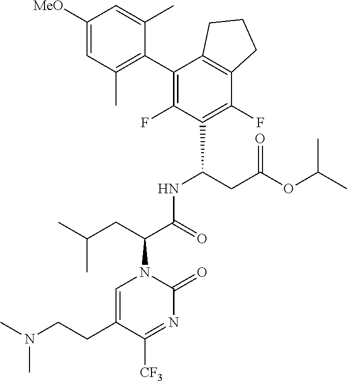 [SMILES: COc1cc(C)c(-c2c(F)c([C@H](CC(=O)OC(C)C)NC(=O)[C@H](CC(C)C)n3cc(CCN(C)C)c(C(F)(F)F)nc3=O)c(F)c3c2CCC3)c(C)c1] |
| | 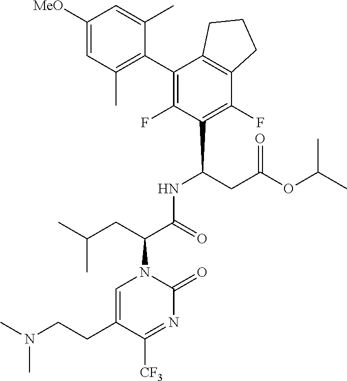 [SMILES: COc1cc(C)c(-c2c(F)c([C@@H](CC(=O)OC(C)C)NC(=O)[C@H](CC(C)C)n3cc(CCN(C)C)c(C(F)(F)F)nc3=O)c(F)c3c2CCC3)c(C)c1] |
| | 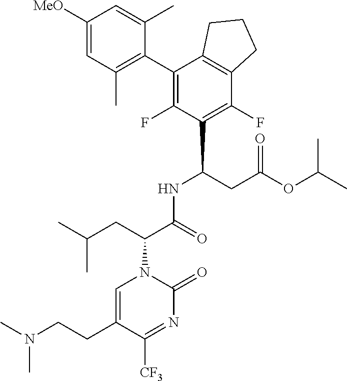 [SMILES: COc1cc(C)c(-c2c(F)c([C@@H](CC(=O)OC(C)C)NC(=O)[C@@H](CC(C)C)n3cc(CCN(C)C)c(C(F)(F)F)nc3=O)c(F)c3c2CCC3)c(C)c1] |
| 315 | 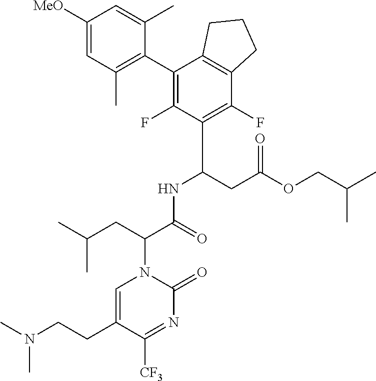 [SMILES: COc1cc(C)c(-c2c(F)c(C(CC(=O)OCC(C)C)NC(=O)C(CC(C)C)n3cc(CCN(C)C)c(C(F)(F)F)nc3=O)c(F)c3c2CCC3)c(C)c1] |
| | 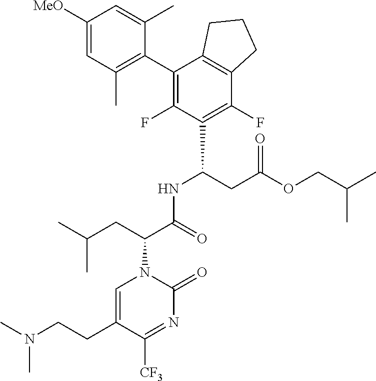 [SMILES: COc1cc(C)c(-c2c(F)c([C@H](CC(=O)OCC(C)C)NC(=O)[C@@H](CC(C)C)n3cc(CCN(C)C)c(C(F)(F)F)nc3=O)c(F)c3c2CCC3)c(C)c1] |
| | 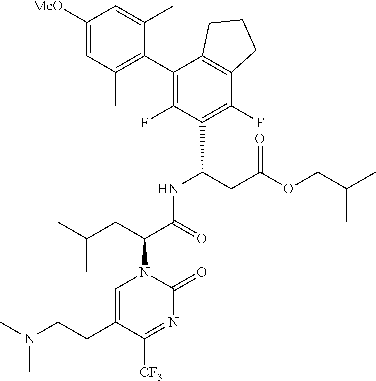 [SMILES: COc1cc(C)c(-c2c(F)c([C@H](CC(=O)OCC(C)C)NC(=O)[C@H](CC(C)C)n3cc(CCN(C)C)c(C(F)(F)F)nc3=O)c(F)c3c2CCC3)c(C)c1] |
| | 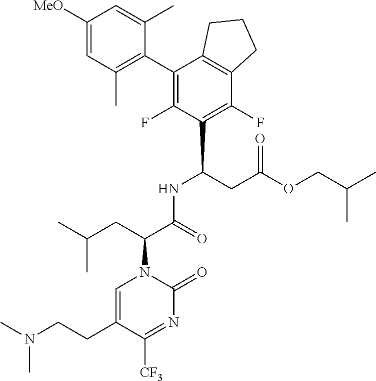 [SMILES: COc1cc(C)c(-c2c(F)c([C@@H](CC(=O)OCC(C)C)NC(=O)[C@H](CC(C)C)n3cc(CCN(C)C)c(C(F)(F)F)nc3=O)c(F)c3c2CCC3)c(C)c1] |
| | 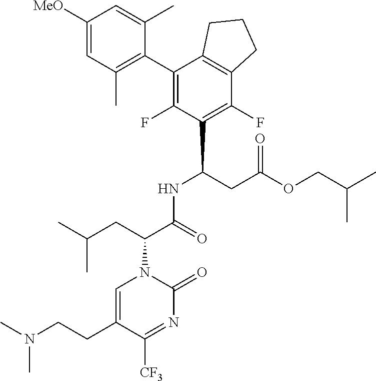 [SMILES: COc1cc(C)c(-c2c(F)c([C@@H](CC(=O)OCC(C)C)NC(=O)[C@@H](CC(C)C)n3cc(CCN(C)C)c(C(F)(F)F)nc3=O)c(F)c3c2CCC3)c(C)c1] |
| 316 | 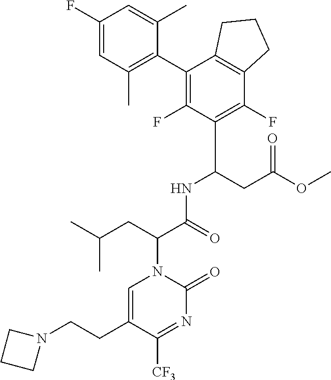 [SMILES: COC(=O)CC(NC(=O)C(CC(C)C)n1cc(CCN2CCC2)c(C(F)(F)F)nc1=O)c1c(F)c2c(c(-c3c(C)cc(F)cc3C)c1F)CCC2] |
| | 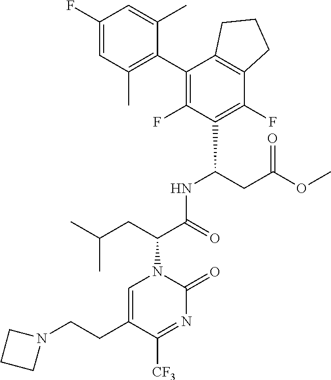 [SMILES: COC(=O)C[C@H](NC(=O)[C@@H](CC(C)C)n1cc(CCN2CCC2)c(C(F)(F)F)nc1=O)c1c(F)c2c(c(-c3c(C)cc(F)cc3C)c1F)CCC2] |
| | 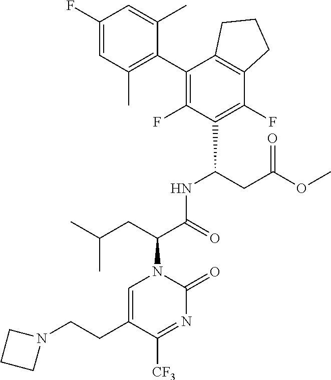 [SMILES: COC(=O)C[C@H](NC(=O)[C@H](CC(C)C)n1cc(CCN2CCC2)c(C(F)(F)F)nc1=O)c1c(F)c2c(c(-c3c(C)cc(F)cc3C)c1F)CCC2] |
| | 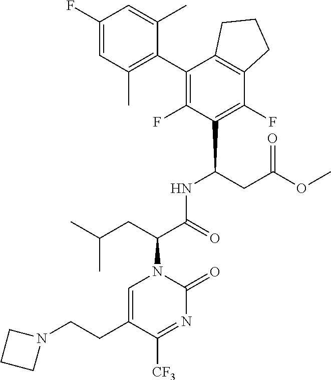 [SMILES: COC(=O)C[C@@H](NC(=O)[C@H](CC(C)C)n1cc(CCN2CCC2)c(C(F)(F)F)nc1=O)c1c(F)c2c(c(-c3c(C)cc(F)cc3C)c1F)CCC2] |
| | 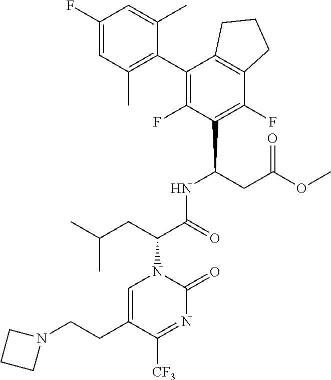 [SMILES: COC(=O)C[C@@H](NC(=O)[C@@H](CC(C)C)n1cc(CCN2CCC2)c(C(F)(F)F)nc1=O)c1c(F)c2c(c(-c3c(C)cc(F)cc3C)c1F)CCC2] |
| 317 | 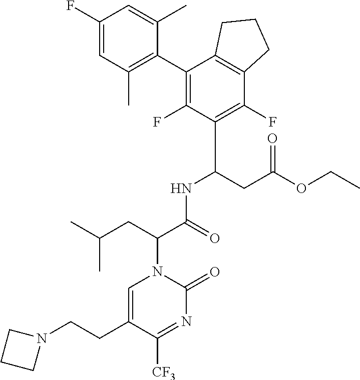 [SMILES: CCOC(=O)CC(NC(=O)C(CC(C)C)n1cc(CCN2CCC2)c(C(F)(F)F)nc1=O)c1c(F)c2c(c(-c3c(C)cc(F)cc3C)c1F)CCC2] |
| | 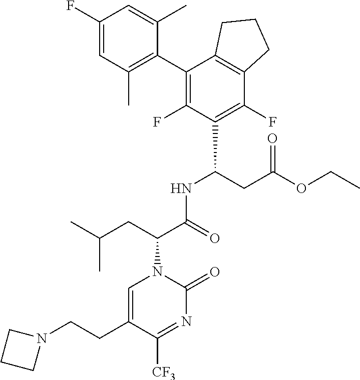 [SMILES: CCOC(=O)C[C@H](NC(=O)[C@@H](CC(C)C)n1cc(CCN2CCC2)c(C(F)(F)F)nc1=O)c1c(F)c2c(c(-c3c(C)cc(F)cc3C)c1F)CCC2] |
| | 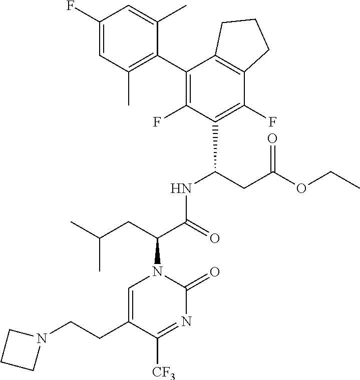 [SMILES: CCOC(=O)C[C@H](NC(=O)[C@H](CC(C)C)n1cc(CCN2CCC2)c(C(F)(F)F)nc1=O)c1c(F)c2c(c(-c3c(C)cc(F)cc3C)c1F)CCC2] |
| | 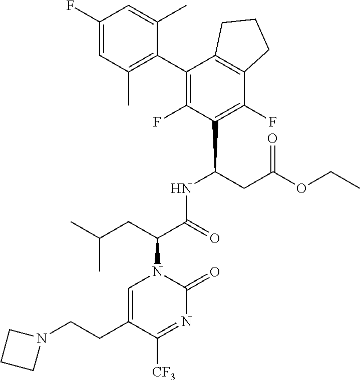 [SMILES: CCOC(=O)C[C@@H](NC(=O)[C@H](CC(C)C)n1cc(CCN2CCC2)c(C(F)(F)F)nc1=O)c1c(F)c2c(c(-c3c(C)cc(F)cc3C)c1F)CCC2] |
| | 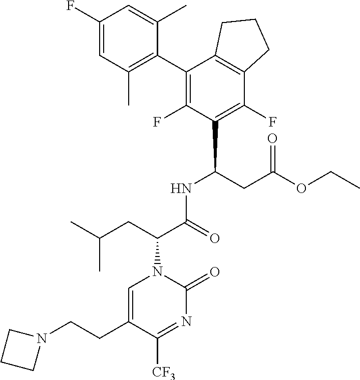 [SMILES: CCOC(=O)C[C@@H](NC(=O)[C@@H](CC(C)C)n1cc(CCN2CCC2)c(C(F)(F)F)nc1=O)c1c(F)c2c(c(-c3c(C)cc(F)cc3C)c1F)CCC2] |
| 318 | 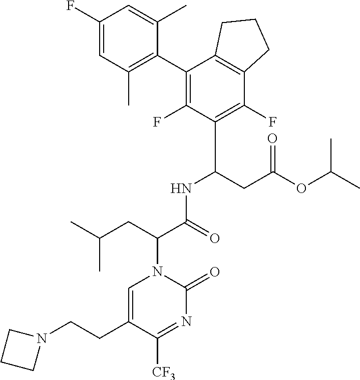 [SMILES: Cc1cc(F)cc(C)c1-c1c(F)c(C(CC(=O)OC(C)C)NC(=O)C(CC(C)C)n2cc(CCN3CCC3)c(C(F)(F)F)nc2=O)c(F)c2c1CCC2] |
| | 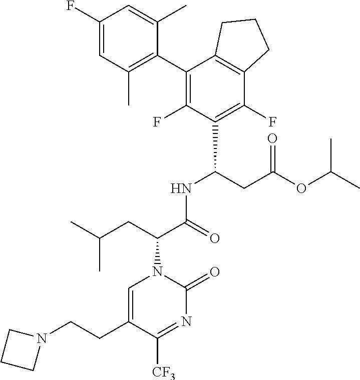 [SMILES: Cc1cc(F)cc(C)c1-c1c(F)c([C@H](CC(=O)OC(C)C)NC(=O)[C@@H](CC(C)C)n2cc(CCN3CCC3)c(C(F)(F)F)nc2=O)c(F)c2c1CCC2] |
| | 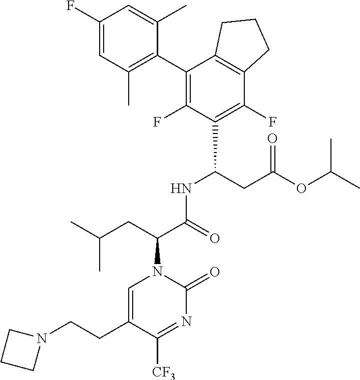 [SMILES: Cc1cc(F)cc(C)c1-c1c(F)c([C@H](CC(=O)OC(C)C)NC(=O)[C@H](CC(C)C)n2cc(CCN3CCC3)c(C(F)(F)F)nc2=O)c(F)c2c1CCC2] |
| | 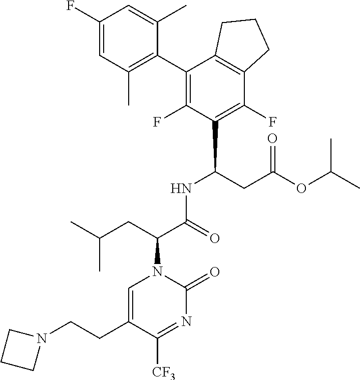 [SMILES: Cc1cc(F)cc(C)c1-c1c(F)c([C@@H](CC(=O)OC(C)C)NC(=O)[C@H](CC(C)C)n2cc(CCN3CCC3)c(C(F)(F)F)nc2=O)c(F)c2c1CCC2] |
| | 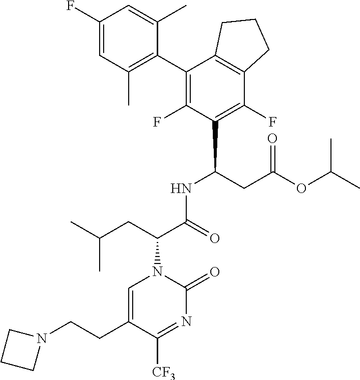 [SMILES: Cc1cc(F)cc(C)c1-c1c(F)c([C@@H](CC(=O)OC(C)C)NC(=O)[C@@H](CC(C)C)n2cc(CCN3CCC3)c(C(F)(F)F)nc2=O)c(F)c2c1CCC2] |
| 319 | 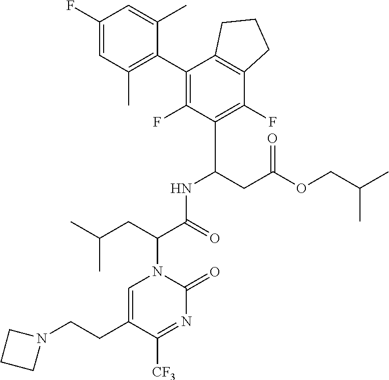 [SMILES: Cc1cc(F)cc(C)c1-c1c(F)c(C(CC(=O)OCC(C)C)NC(=O)C(CC(C)C)n2cc(CCN3CCC3)c(C(F)(F)F)nc2=O)c(F)c2c1CCC2] |
| | 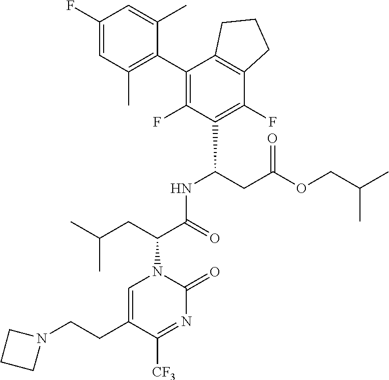 [SMILES: Cc1cc(F)cc(C)c1-c1c(F)c([C@H](CC(=O)OCC(C)C)NC(=O)[C@@H](CC(C)C)n2cc(CCN3CCC3)c(C(F)(F)F)nc2=O)c(F)c2c1CCC2] |
| | 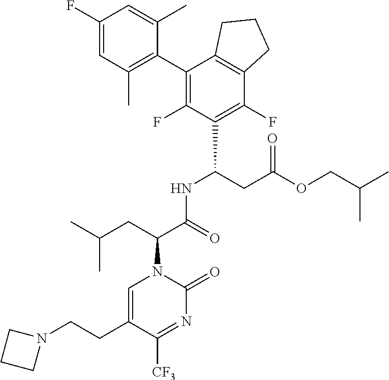 [SMILES: Cc1cc(F)cc(C)c1-c1c(F)c([C@H](CC(=O)OCC(C)C)NC(=O)[C@H](CC(C)C)n2cc(CCN3CCC3)c(C(F)(F)F)nc2=O)c(F)c2c1CCC2] |
| | 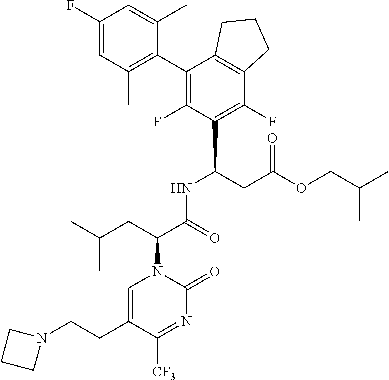 [SMILES: Cc1cc(F)cc(C)c1-c1c(F)c([C@@H](CC(=O)OCC(C)C)NC(=O)[C@H](CC(C)C)n2cc(CCN3CCC3)c(C(F)(F)F)nc2=O)c(F)c2c1CCC2] |
| | 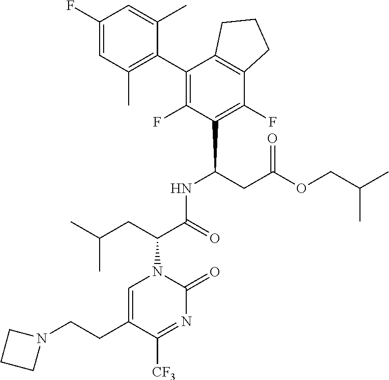 [SMILES: Cc1cc(F)cc(C)c1-c1c(F)c([C@@H](CC(=O)OCC(C)C)NC(=O)[C@@H](CC(C)C)n2cc(CCN3CCC3)c(C(F)(F)F)nc2=O)c(F)c2c1CCC2] |
| 320 | 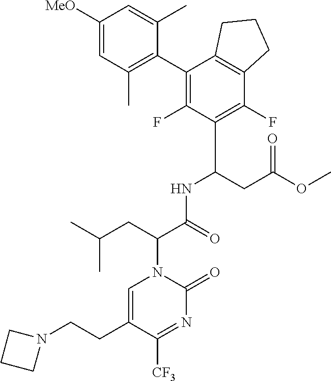 [SMILES: COC(=O)CC(NC(=O)C(CC(C)C)n1cc(CCN2CCC2)c(C(F)(F)F)nc1=O)c1c(F)c2c(c(-c3c(C)cc(OC)cc3C)c1F)CCC2] |
| | 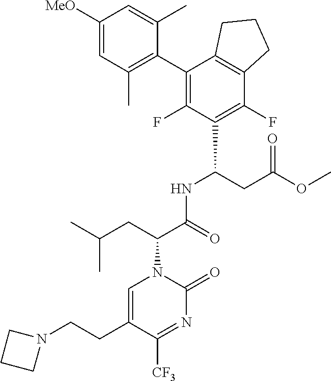 [SMILES: COC(=O)C[C@H](NC(=O)[C@@H](CC(C)C)n1cc(CCN2CCC2)c(C(F)(F)F)nc1=O)c1c(F)c2c(c(-c3c(C)cc(OC)cc3C)c1F)CCC2] |
| | 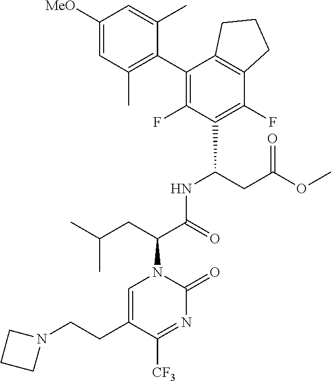 [SMILES: COC(=O)C[C@H](NC(=O)[C@H](CC(C)C)n1cc(CCN2CCC2)c(C(F)(F)F)nc1=O)c1c(F)c2c(c(-c3c(C)cc(OC)cc3C)c1F)CCC2] |
| | 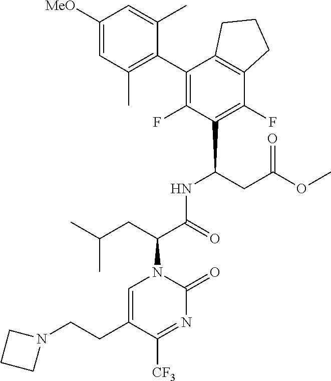 [SMILES: COC(=O)C[C@@H](NC(=O)[C@H](CC(C)C)n1cc(CCN2CCC2)c(C(F)(F)F)nc1=O)c1c(F)c2c(c(-c3c(C)cc(OC)cc3C)c1F)CCC2] |
| | 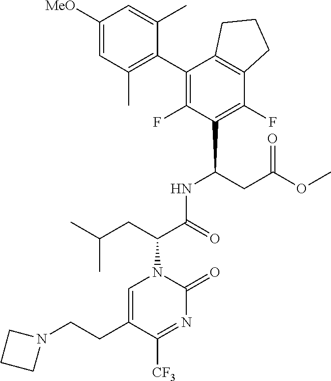 [SMILES: COC(=O)C[C@@H](NC(=O)[C@@H](CC(C)C)n1cc(CCN2CCC2)c(C(F)(F)F)nc1=O)c1c(F)c2c(c(-c3c(C)cc(OC)cc3C)c1F)CCC2] |
| 321 | 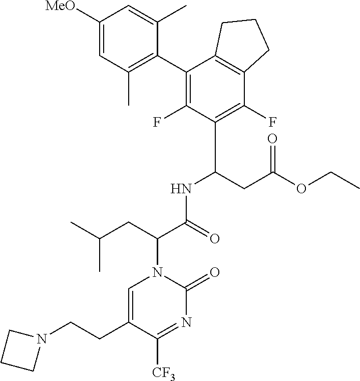 [SMILES: CCOC(=O)CC(NC(=O)C(CC(C)C)n1cc(CCN2CCC2)c(C(F)(F)F)nc1=O)c1c(F)c2c(c(-c3c(C)cc(OC)cc3C)c1F)CCC2] |
| | 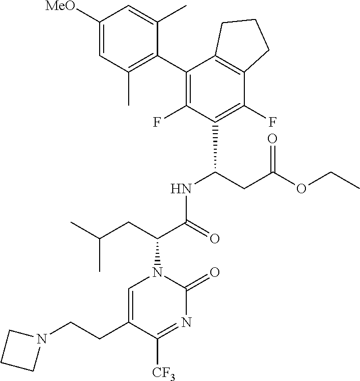 [SMILES: CCOC(=O)C[C@H](NC(=O)[C@@H](CC(C)C)n1cc(CCN2CCC2)c(C(F)(F)F)nc1=O)c1c(F)c2c(c(-c3c(C)cc(OC)cc3C)c1F)CCC2] |
| | 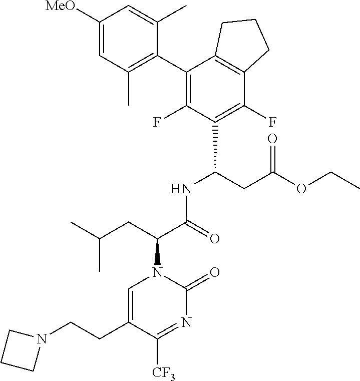 [SMILES: CCOC(=O)C[C@H](NC(=O)[C@H](CC(C)C)n1cc(CCN2CCC2)c(C(F)(F)F)nc1=O)c1c(F)c2c(c(-c3c(C)cc(OC)cc3C)c1F)CCC2] |
| | 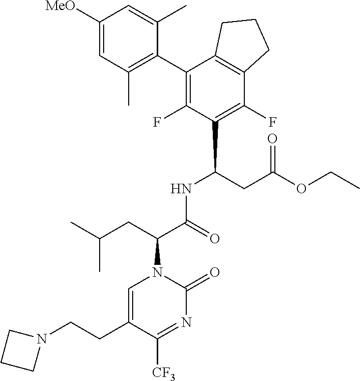 [SMILES: CCOC(=O)C[C@@H](NC(=O)[C@H](CC(C)C)n1cc(CCN2CCC2)c(C(F)(F)F)nc1=O)c1c(F)c2c(c(-c3c(C)cc(OC)cc3C)c1F)CCC2] |
| | 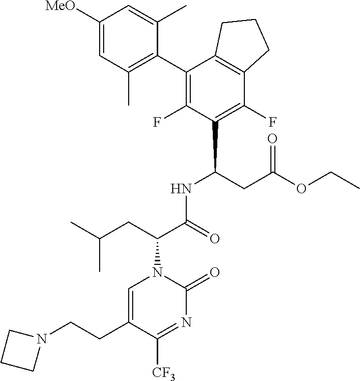 [SMILES: CCOC(=O)C[C@@H](NC(=O)[C@@H](CC(C)C)n1cc(CCN2CCC2)c(C(F)(F)F)nc1=O)c1c(F)c2c(c(-c3c(C)cc(OC)cc3C)c1F)CCC2] |
| 322 | 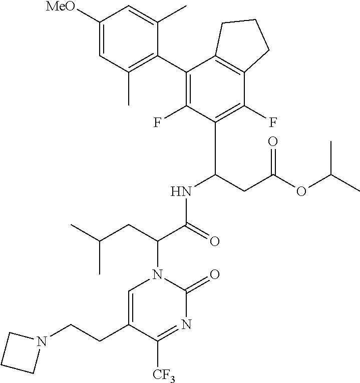 [SMILES: COc1cc(C)c(-c2c(F)c(C(CC(=O)OC(C)C)NC(=O)C(CC(C)C)n3cc(CCN4CCC4)c(C(F)(F)F)nc3=O)c(F)c3c2CCC3)c(C)c1] |
| | 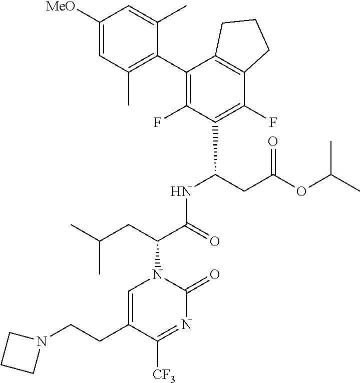 [SMILES: COc1cc(C)c(-c2c(F)c([C@H](CC(=O)OC(C)C)NC(=O)[C@@H](CC(C)C)n3cc(CCN4CCC4)c(C(F)(F)F)nc3=O)c(F)c3c2CCC3)c(C)c1] |
| | 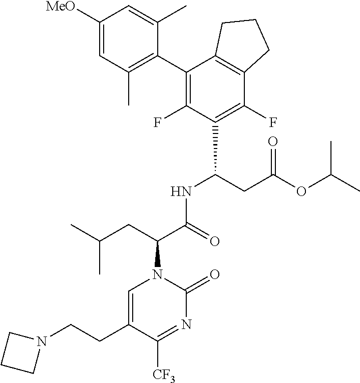 [SMILES: COc1cc(C)c(-c2c(F)c([C@H](CC(=O)OC(C)C)NC(=O)[C@H](CC(C)C)n3cc(CCN4CCC4)c(C(F)(F)F)nc3=O)c(F)c3c2CCC3)c(C)c1] |
| | 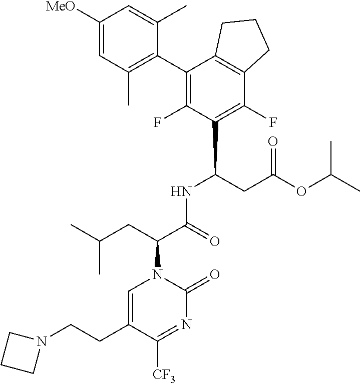 [SMILES: COc1cc(C)c(-c2c(F)c([C@@H](CC(=O)OC(C)C)NC(=O)[C@H](CC(C)C)n3cc(CCN4CCC4)c(C(F)(F)F)nc3=O)c(F)c3c2CCC3)c(C)c1] |
| | 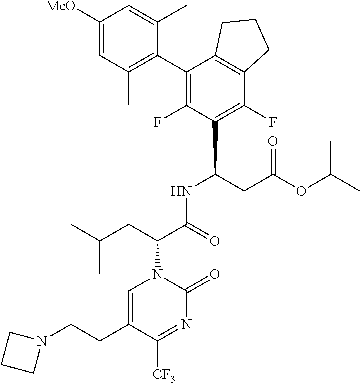 [SMILES: COc1cc(C)c(-c2c(F)c([C@@H](CC(=O)OC(C)C)NC(=O)[C@@H](CC(C)C)n3cc(CCN4CCC4)c(C(F)(F)F)nc3=O)c(F)c3c2CCC3)c(C)c1] |
| 323 | 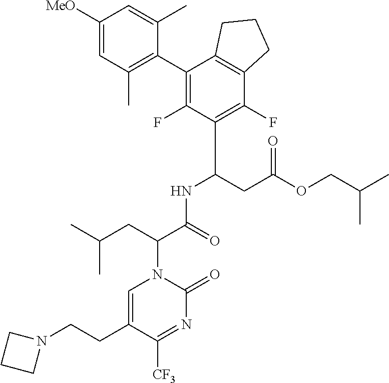 [SMILES: COc1cc(C)c(-c2c(F)c(C(CC(=O)OCC(C)C)NC(=O)C(CC(C)C)n3cc(CCN4CCC4)c(C(F)(F)F)nc3=O)c(F)c3c2CCC3)c(C)c1] |
| | 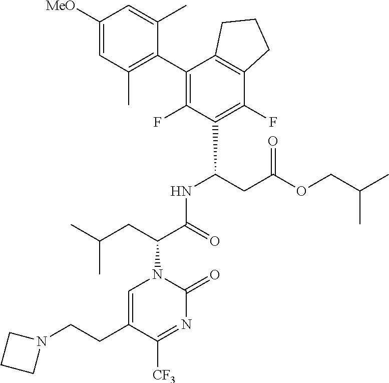 [SMILES: COc1cc(C)c(-c2c(F)c([C@H](CC(=O)OCC(C)C)NC(=O)[C@@H](CC(C)C)n3cc(CCN4CCC4)c(C(F)(F)F)nc3=O)c(F)c3c2CCC3)c(C)c1] |
| | 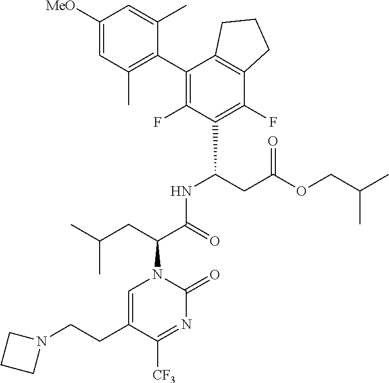 [SMILES: COc1cc(C)c(-c2c(F)c([C@H](CC(=O)OCC(C)C)NC(=O)[C@H](CC(C)C)n3cc(CCN4CCC4)c(C(F)(F)F)nc3=O)c(F)c3c2CCC3)c(C)c1] |
| | 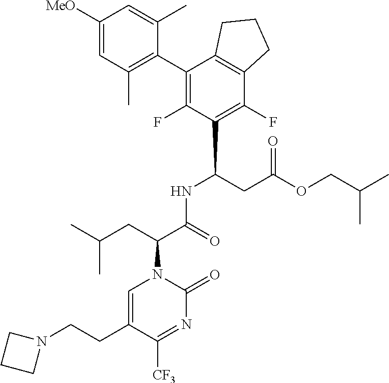 [SMILES: COc1cc(C)c(-c2c(F)c([C@@H](CC(=O)OCC(C)C)NC(=O)[C@H](CC(C)C)n3cc(CCN4CCC4)c(C(F)(F)F)nc3=O)c(F)c3c2CCC3)c(C)c1] |
| | 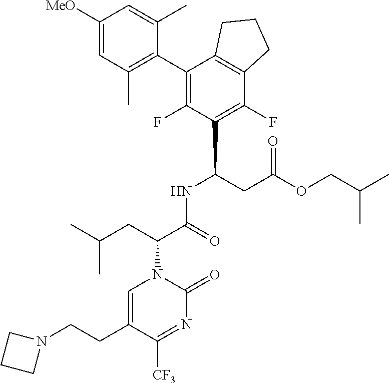 [SMILES: COc1cc(C)c(-c2c(F)c([C@@H](CC(=O)OCC(C)C)NC(=O)[C@@H](CC(C)C)n3cc(CCN4CCC4)c(C(F)(F)F)nc3=O)c(F)c3c2CCC3)c(C)c1] |
| 324 | 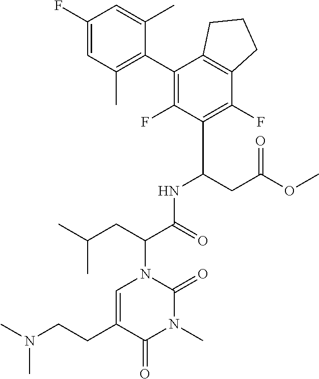 |
| | 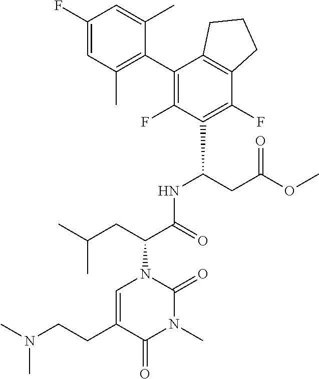 |
| | 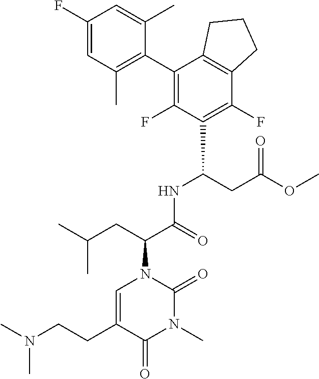 |
| | 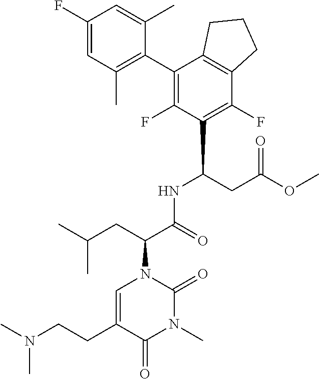 |
| | 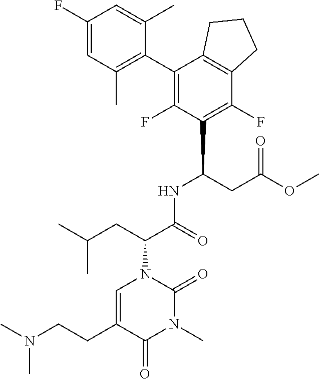 |
| 325 | 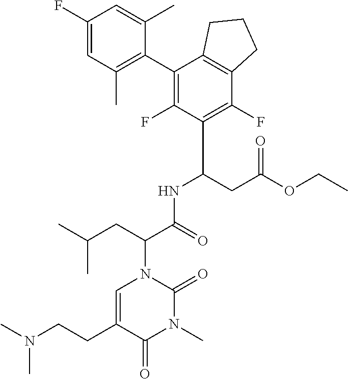 [SMILES: CCOC(=O)CC(NC(=O)C(CC(C)C)n1cc(CCN(C)C)c(=O)n(C)c1=O)c1c(F)c2c(c(-c3c(C)cc(F)cc3C)c1F)CCC2] |
| | 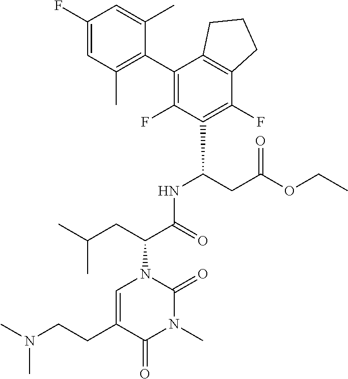 [SMILES: CCOC(=O)C[C@H](NC(=O)[C@@H](CC(C)C)n1cc(CCN(C)C)c(=O)n(C)c1=O)c1c(F)c2c(c(-c3c(C)cc(F)cc3C)c1F)CCC2] |
| | 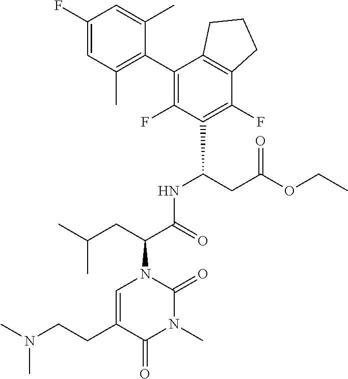 [SMILES: CCOC(=O)C[C@H](NC(=O)[C@H](CC(C)C)n1cc(CCN(C)C)c(=O)n(C)c1=O)c1c(F)c2c(c(-c3c(C)cc(F)cc3C)c1F)CCC2] |
| | 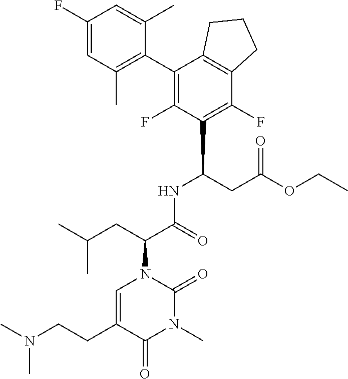 [SMILES: CCOC(=O)C[C@@H](NC(=O)[C@H](CC(C)C)n1cc(CCN(C)C)c(=O)n(C)c1=O)c1c(F)c2c(c(-c3c(C)cc(F)cc3C)c1F)CCC2] |
| | 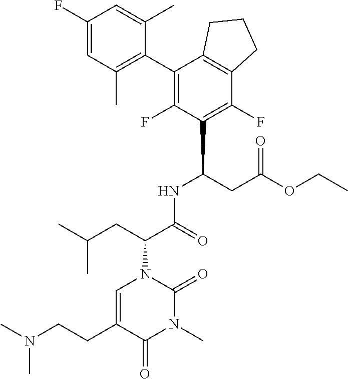 [SMILES: CCOC(=O)C[C@@H](NC(=O)[C@@H](CC(C)C)n1cc(CCN(C)C)c(=O)n(C)c1=O)c1c(F)c2c(c(-c3c(C)cc(F)cc3C)c1F)CCC2] |
| 326 | 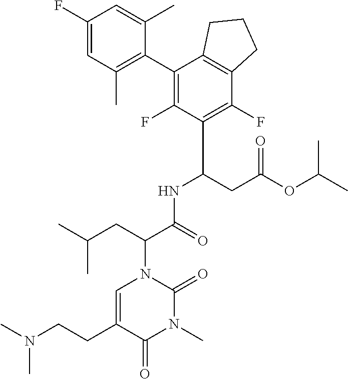 [SMILES: Cc1cc(F)cc(C)c1-c1c(F)c(C(CC(=O)OC(C)C)NC(=O)C(CC(C)C)n2cc(CCN(C)C)c(=O)n(C)c2=O)c(F)c2c1CCC2] |
| | 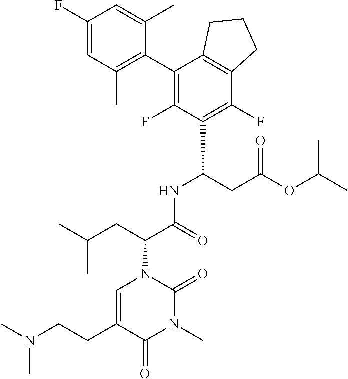 [SMILES: Cc1cc(F)cc(C)c1-c1c(F)c([C@H](CC(=O)OC(C)C)NC(=O)[C@@H](CC(C)C)n2cc(CCN(C)C)c(=O)n(C)c2=O)c(F)c2c1CCC2] |
| | 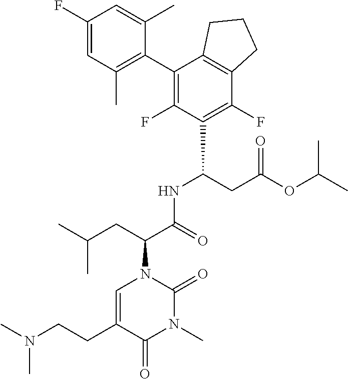 [SMILES: Cc1cc(F)cc(C)c1-c1c(F)c([C@H](CC(=O)OC(C)C)NC(=O)[C@H](CC(C)C)n2cc(CCN(C)C)c(=O)n(C)c2=O)c(F)c2c1CCC2] |
| | 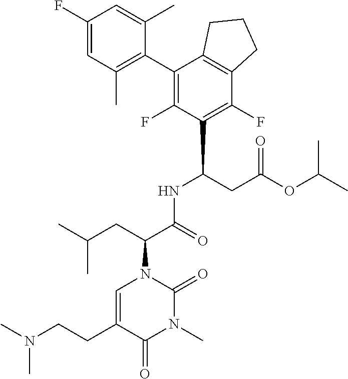 [SMILES: Cc1cc(F)cc(C)c1-c1c(F)c([C@@H](CC(=O)OC(C)C)NC(=O)[C@H](CC(C)C)n2cc(CCN(C)C)c(=O)n(C)c2=O)c(F)c2c1CCC2] |
| | 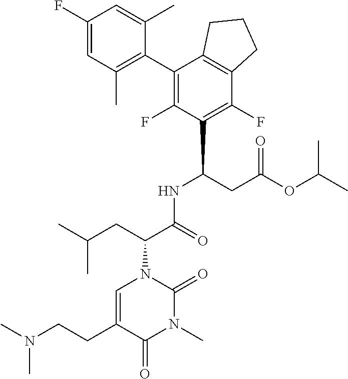 [SMILES: Cc1cc(F)cc(C)c1-c1c(F)c([C@@H](CC(=O)OC(C)C)NC(=O)[C@@H](CC(C)C)n2cc(CCN(C)C)c(=O)n(C)c2=O)c(F)c2c1CCC2] |
| 327 | 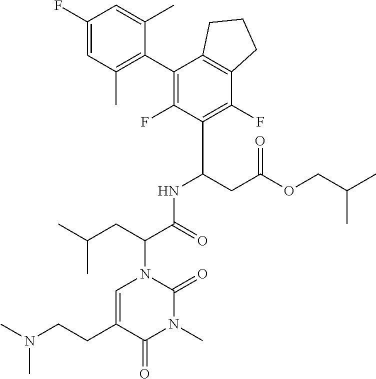 [SMILES: Cc1cc(F)cc(C)c1-c1c(F)c(C(CC(=O)OCC(C)C)NC(=O)C(CC(C)C)n2cc(CCN(C)C)c(=O)n(C)c2=O)c(F)c2c1CCC2] |
| | 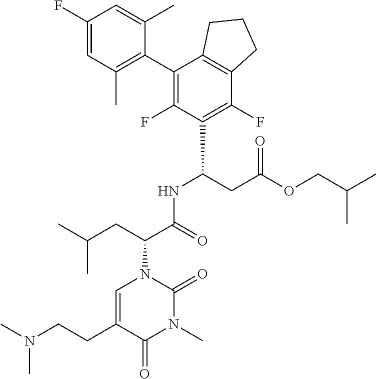 [SMILES: Cc1cc(F)cc(C)c1-c1c(F)c([C@H](CC(=O)OCC(C)C)NC(=O)[C@@H](CC(C)C)n2cc(CCN(C)C)c(=O)n(C)c2=O)c(F)c2c1CCC2] |
| | 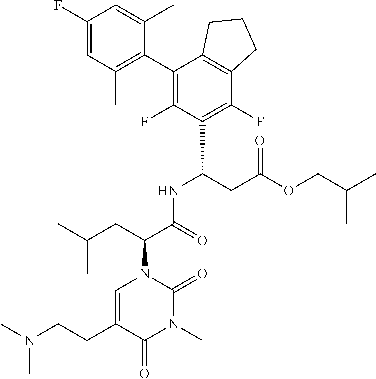 [SMILES: Cc1cc(F)cc(C)c1-c1c(F)c([C@H](CC(=O)OCC(C)C)NC(=O)[C@H](CC(C)C)n2cc(CCN(C)C)c(=O)n(C)c2=O)c(F)c2c1CCC2] |
| | 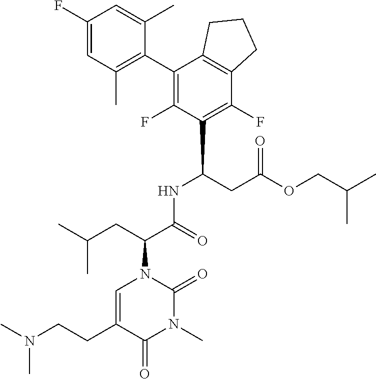 [SMILES: Cc1cc(F)cc(C)c1-c1c(F)c([C@@H](CC(=O)OCC(C)C)NC(=O)[C@H](CC(C)C)n2cc(CCN(C)C)c(=O)n(C)c2=O)c(F)c2c1CCC2] |
| | 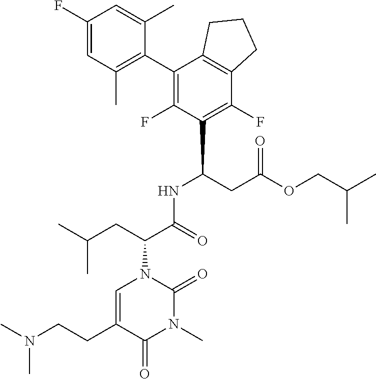 [SMILES: Cc1cc(F)cc(C)c1-c1c(F)c([C@@H](CC(=O)OCC(C)C)NC(=O)[C@@H](CC(C)C)n2cc(CCN(C)C)c(=O)n(C)c2=O)c(F)c2c1CCC2] |
| 328 | 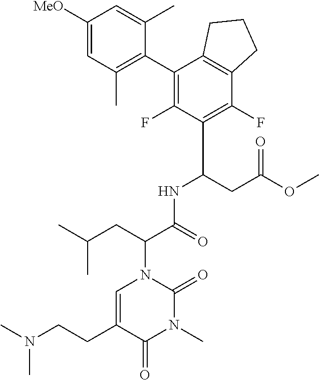 |
| | 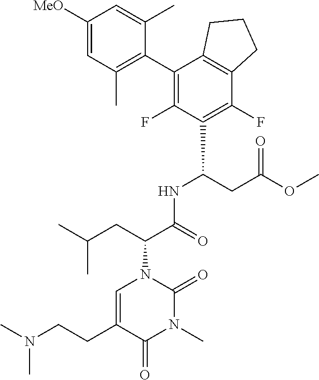 |
| | 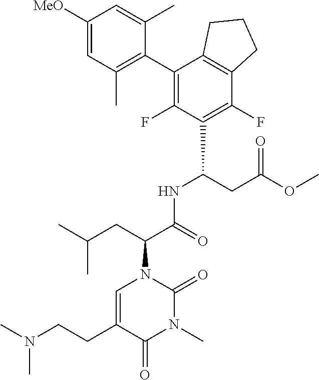 |
| | 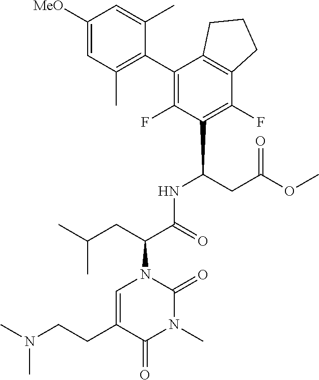 |
| | 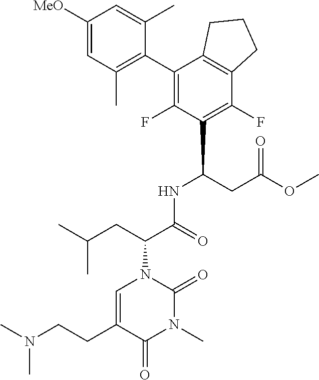 |
| 329 | 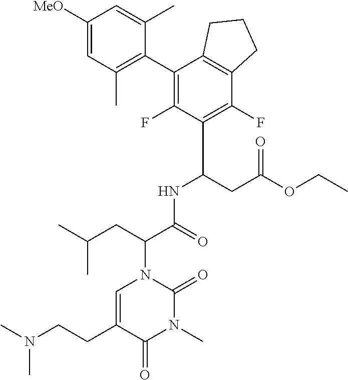 [SMILES: CCOC(=O)CC(NC(=O)C(CC(C)C)n1cc(CCN(C)C)c(=O)n(C)c1=O)c1c(F)c2c(c(-c3c(C)cc(OC)cc3C)c1F)CCC2] |
| | 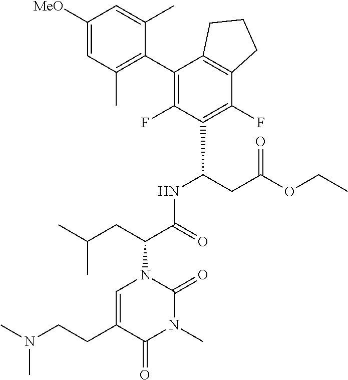 [SMILES: CCOC(=O)C[C@H](NC(=O)[C@@H](CC(C)C)n1cc(CCN(C)C)c(=O)n(C)c1=O)c1c(F)c2c(c(-c3c(C)cc(OC)cc3C)c1F)CCC2] |
| | 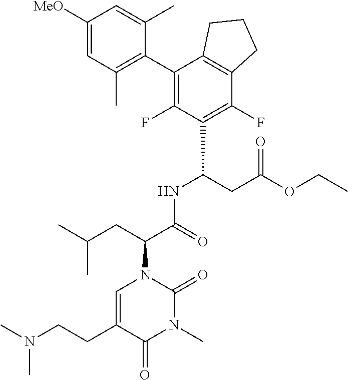 [SMILES: CCOC(=O)C[C@H](NC(=O)[C@H](CC(C)C)n1cc(CCN(C)C)c(=O)n(C)c1=O)c1c(F)c2c(c(-c3c(C)cc(OC)cc3C)c1F)CCC2] |
| | 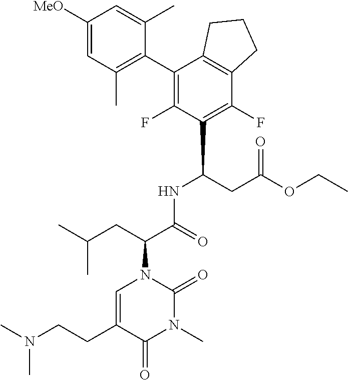 [SMILES: CCOC(=O)C[C@@H](NC(=O)[C@H](CC(C)C)n1cc(CCN(C)C)c(=O)n(C)c1=O)c1c(F)c2c(c(-c3c(C)cc(OC)cc3C)c1F)CCC2] |
| | 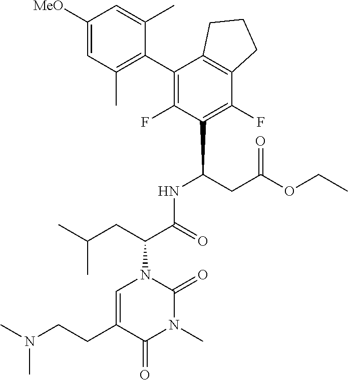 [SMILES: CCOC(=O)C[C@@H](NC(=O)[C@@H](CC(C)C)n1cc(CCN(C)C)c(=O)n(C)c1=O)c1c(F)c2c(c(-c3c(C)cc(OC)cc3C)c1F)CCC2] |
| 330 | 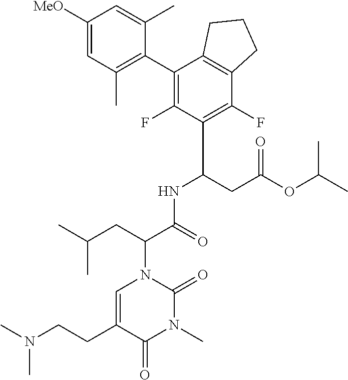 [SMILES: COc1cc(C)c(-c2c(F)c(C(CC(=O)OC(C)C)NC(=O)C(CC(C)C)n3cc(CCN(C)C)c(=O)n(C)c3=O)c(F)c3c2CCC3)c(C)c1] |
| | 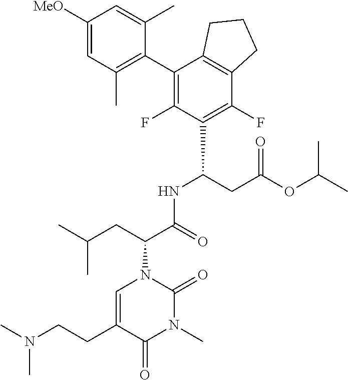 [SMILES: COc1cc(C)c(-c2c(F)c([C@H](CC(=O)OC(C)C)NC(=O)[C@@H](CC(C)C)n3cc(CCN(C)C)c(=O)n(C)c3=O)c(F)c3c2CCC3)c(C)c1] |
| | 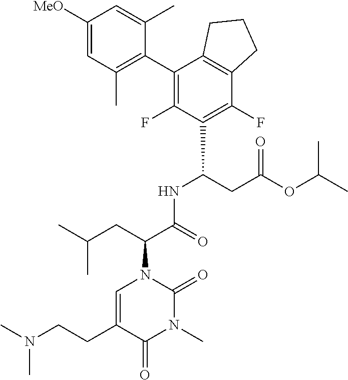 [SMILES: COc1cc(C)c(-c2c(F)c([C@H](CC(=O)OC(C)C)NC(=O)[C@H](CC(C)C)n3cc(CCN(C)C)c(=O)n(C)c3=O)c(F)c3c2CCC3)c(C)c1] |
| | 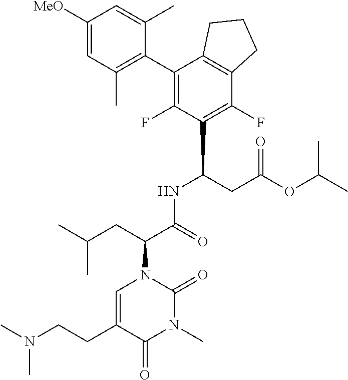 [SMILES: COc1cc(C)c(-c2c(F)c([C@@H](CC(=O)OC(C)C)NC(=O)[C@H](CC(C)C)n3cc(CCN(C)C)c(=O)n(C)c3=O)c(F)c3c2CCC3)c(C)c1] |
| | 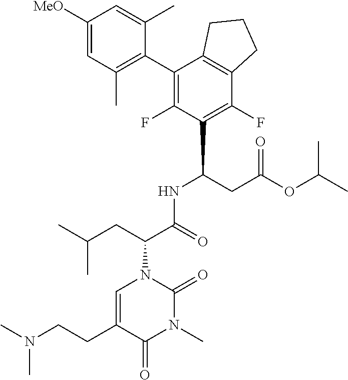 [SMILES: COc1cc(C)c(-c2c(F)c([C@@H](CC(=O)OC(C)C)NC(=O)[C@@H](CC(C)C)n3cc(CCN(C)C)c(=O)n(C)c3=O)c(F)c3c2CCC3)c(C)c1] |
| 331 | 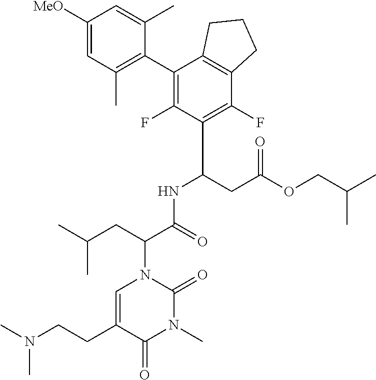 [SMILES: COc1cc(C)c(-c2c(F)c(C(CC(=O)OCC(C)C)NC(=O)C(CC(C)C)n3cc(CCN(C)C)c(=O)n(C)c3=O)c(F)c3c2CCC3)c(C)c1] |
| | 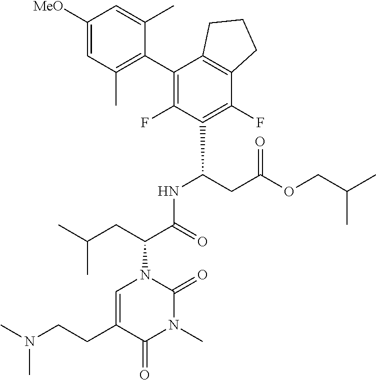 [SMILES: COc1cc(C)c(-c2c(F)c([C@H](CC(=O)OCC(C)C)NC(=O)[C@@H](CC(C)C)n3cc(CCN(C)C)c(=O)n(C)c3=O)c(F)c3c2CCC3)c(C)c1] |
| | 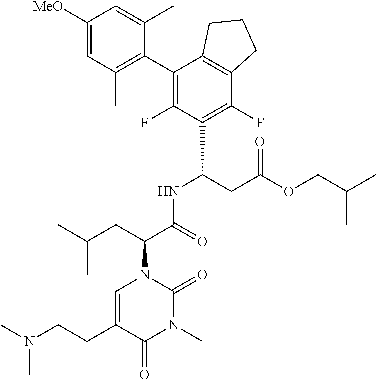 [SMILES: COc1cc(C)c(-c2c(F)c([C@H](CC(=O)OCC(C)C)NC(=O)[C@H](CC(C)C)n3cc(CCN(C)C)c(=O)n(C)c3=O)c(F)c3c2CCC3)c(C)c1] |
| | 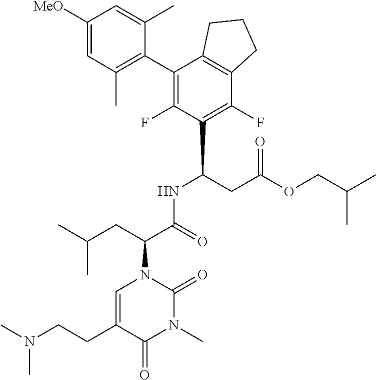 [SMILES: COc1cc(C)c(-c2c(F)c([C@@H](CC(=O)OCC(C)C)NC(=O)[C@H](CC(C)C)n3cc(CCN(C)C)c(=O)n(C)c3=O)c(F)c3c2CCC3)c(C)c1] |
| | 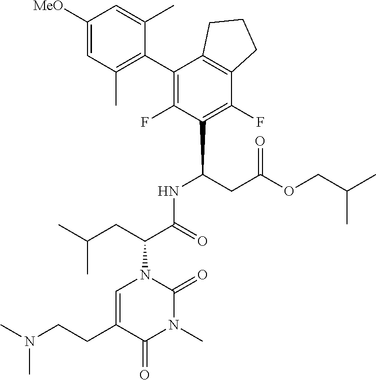 [SMILES: COc1cc(C)c(-c2c(F)c([C@@H](CC(=O)OCC(C)C)NC(=O)[C@@H](CC(C)C)n3cc(CCN(C)C)c(=O)n(C)c3=O)c(F)c3c2CCC3)c(C)c1] |
| 332 | 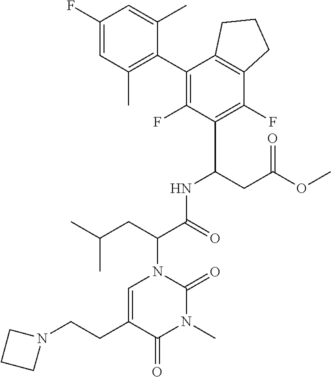 [SMILES: COC(=O)CC(NC(=O)C(CC(C)C)n1cc(CCN2CCC2)c(=O)n(C)c1=O)c1c(F)c2c(c(-c3c(C)cc(F)cc3C)c1F)CCC2] |
| | 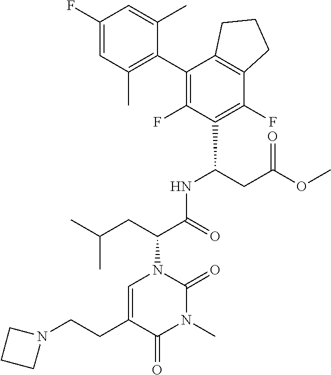 [SMILES: COC(=O)C[C@H](NC(=O)[C@@H](CC(C)C)n1cc(CCN2CCC2)c(=O)n(C)c1=O)c1c(F)c2c(c(-c3c(C)cc(F)cc3C)c1F)CCC2] |
| | 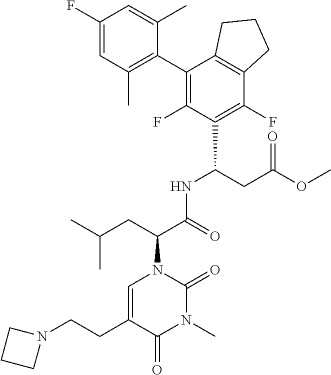 [SMILES: COC(=O)C[C@H](NC(=O)[C@H](CC(C)C)n1cc(CCN2CCC2)c(=O)n(C)c1=O)c1c(F)c2c(c(-c3c(C)cc(F)cc3C)c1F)CCC2] |
| | 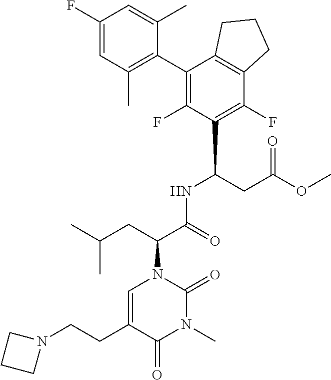 [SMILES: COC(=O)C[C@@H](NC(=O)[C@H](CC(C)C)n1cc(CCN2CCC2)c(=O)n(C)c1=O)c1c(F)c2c(c(-c3c(C)cc(F)cc3C)c1F)CCC2] |
| | 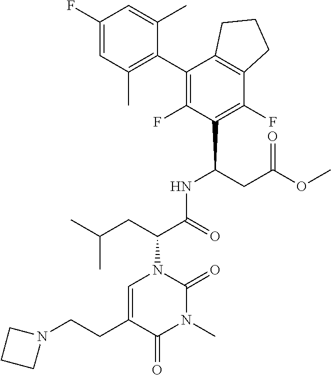 [SMILES: COC(=O)C[C@@H](NC(=O)[C@@H](CC(C)C)n1cc(CCN2CCC2)c(=O)n(C)c1=O)c1c(F)c2c(c(-c3c(C)cc(F)cc3C)c1F)CCC2] |
| 333 | 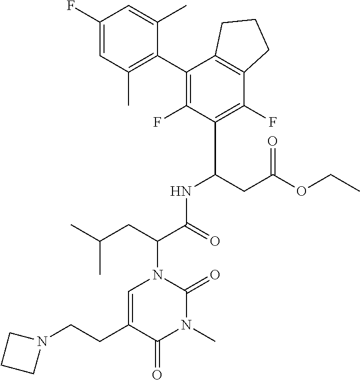 [SMILES: CCOC(=O)CC(NC(=O)C(CC(C)C)n1cc(CCN2CCC2)c(=O)n(C)c1=O)c1c(F)c2c(c(-c3c(C)cc(F)cc3C)c1F)CCC2] |
| | 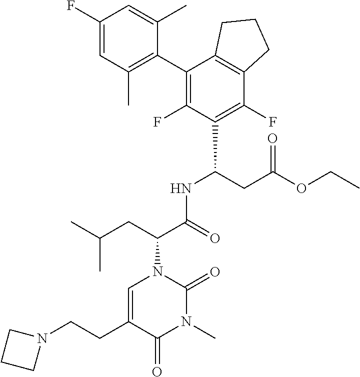 [SMILES: CCOC(=O)C[C@H](NC(=O)[C@@H](CC(C)C)n1cc(CCN2CCC2)c(=O)n(C)c1=O)c1c(F)c2c(c(-c3c(C)cc(F)cc3C)c1F)CCC2] |
| | 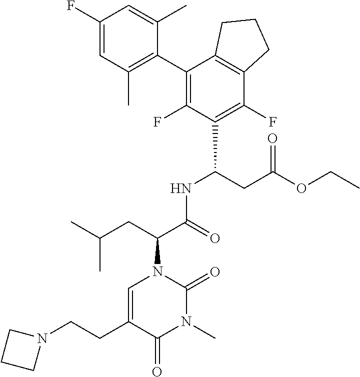 [SMILES: CCOC(=O)C[C@H](NC(=O)[C@H](CC(C)C)n1cc(CCN2CCC2)c(=O)n(C)c1=O)c1c(F)c2c(c(-c3c(C)cc(F)cc3C)c1F)CCC2] |
| | 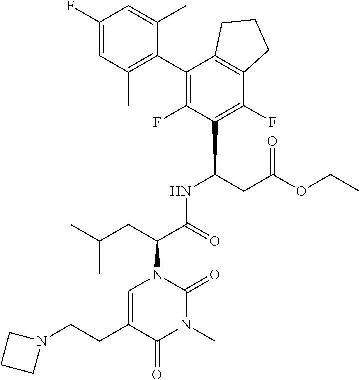 [SMILES: CCOC(=O)C[C@@H](NC(=O)[C@H](CC(C)C)n1cc(CCN2CCC2)c(=O)n(C)c1=O)c1c(F)c2c(c(-c3c(C)cc(F)cc3C)c1F)CCC2] |
| | 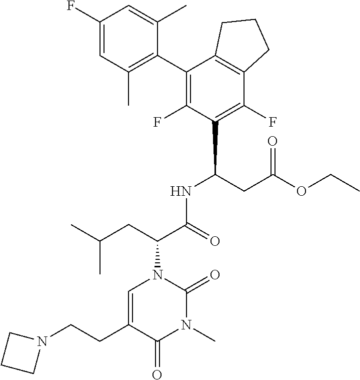 [SMILES: CCOC(=O)C[C@@H](NC(=O)[C@@H](CC(C)C)n1cc(CCN2CCC2)c(=O)n(C)c1=O)c1c(F)c2c(c(-c3c(C)cc(F)cc3C)c1F)CCC2] |
| 334 | 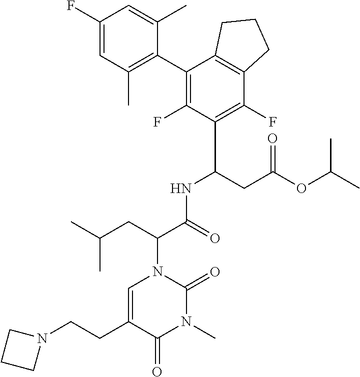 [SMILES: Cc1cc(F)cc(C)c1-c1c(F)c(C(CC(=O)OC(C)C)NC(=O)C(CC(C)C)n2cc(CCN3CCC3)c(=O)n(C)c2=O)c(F)c2c1CCC2] |
| | 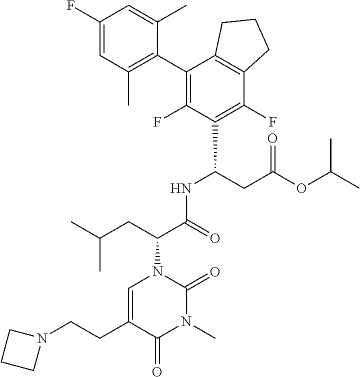 [SMILES: Cc1cc(F)cc(C)c1-c1c(F)c([C@H](CC(=O)OC(C)C)NC(=O)[C@@H](CC(C)C)n2cc(CCN3CCC3)c(=O)n(C)c2=O)c(F)c2c1CCC2] |
| | 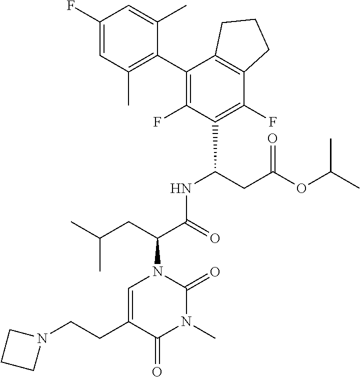 [SMILES: Cc1cc(F)cc(C)c1-c1c(F)c([C@H](CC(=O)OC(C)C)NC(=O)[C@H](CC(C)C)n2cc(CCN3CCC3)c(=O)n(C)c2=O)c(F)c2c1CCC2] |
| | 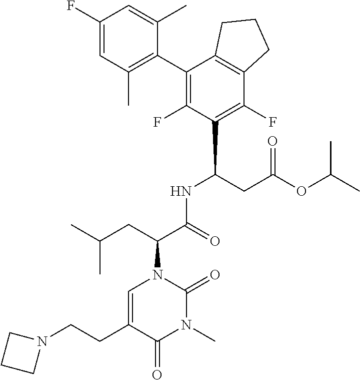 [SMILES: Cc1cc(F)cc(C)c1-c1c(F)c([C@@H](CC(=O)OC(C)C)NC(=O)[C@H](CC(C)C)n2cc(CCN3CCC3)c(=O)n(C)c2=O)c(F)c2c1CCC2] |
| | 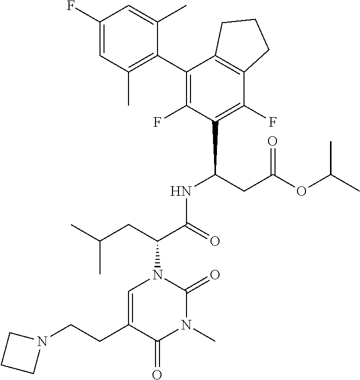 [SMILES: Cc1cc(F)cc(C)c1-c1c(F)c([C@@H](CC(=O)OC(C)C)NC(=O)[C@@H](CC(C)C)n2cc(CCN3CCC3)c(=O)n(C)c2=O)c(F)c2c1CCC2] |
| 335 | 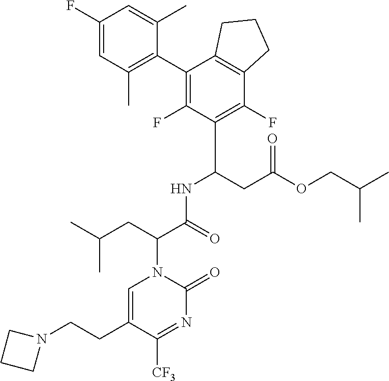 [SMILES: Cc1cc(F)cc(C)c1-c1c(F)c(C(CC(=O)OCC(C)C)NC(=O)C(CC(C)C)n2cc(CCN3CCC3)c(C(F)(F)F)nc2=O)c(F)c2c1CCC2] |
| | 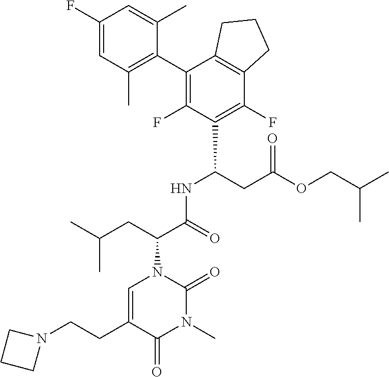 [SMILES: Cc1cc(F)cc(C)c1-c1c(F)c([C@H](CC(=O)OCC(C)C)NC(=O)[C@@H](CC(C)C)n2cc(CCN3CCC3)c(=O)n(C)c2=O)c(F)c2c1CCC2] |
| | 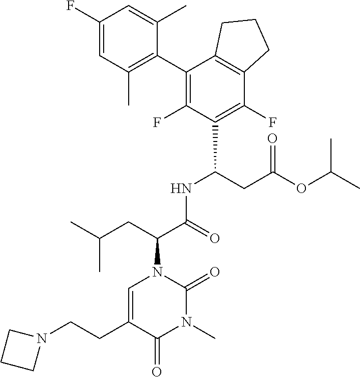 [SMILES: Cc1cc(F)cc(C)c1-c1c(F)c([C@H](CC(=O)OC(C)C)NC(=O)[C@H](CC(C)C)n2cc(CCN3CCC3)c(=O)n(C)c2=O)c(F)c2c1CCC2] |
| | 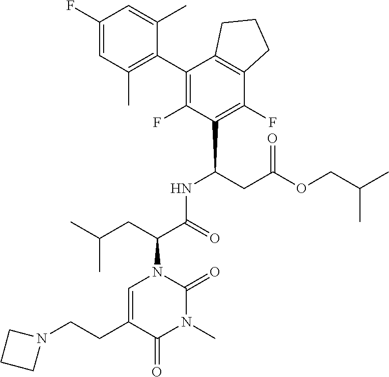 [SMILES: Cc1cc(F)cc(C)c1-c1c(F)c([C@@H](CC(=O)OCC(C)C)NC(=O)[C@H](CC(C)C)n2cc(CCN3CCC3)c(=O)n(C)c2=O)c(F)c2c1CCC2] |
| | 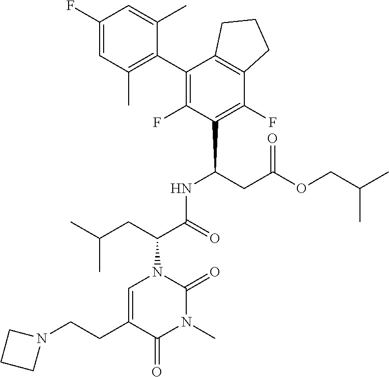 [SMILES: Cc1cc(F)cc(C)c1-c1c(F)c([C@@H](CC(=O)OCC(C)C)NC(=O)[C@@H](CC(C)C)n2cc(CCN3CCC3)c(=O)n(C)c2=O)c(F)c2c1CCC2] |
| 336 | 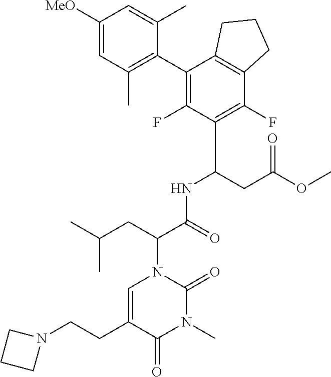 [SMILES: COC(=O)CC(NC(=O)C(CC(C)C)n1cc(CCN2CCC2)c(=O)n(C)c1=O)c1c(F)c2c(c(-c3c(C)cc(OC)cc3C)c1F)CCC2] |
| | 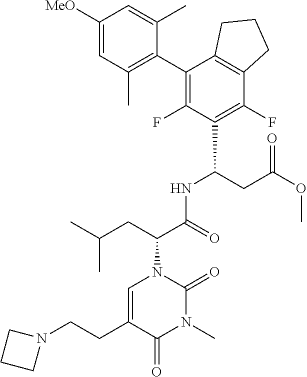 [SMILES: COC(=O)C[C@H](NC(=O)[C@@H](CC(C)C)n1cc(CCN2CCC2)c(=O)n(C)c1=O)c1c(F)c2c(c(-c3c(C)cc(OC)cc3C)c1F)CCC2] |
| | 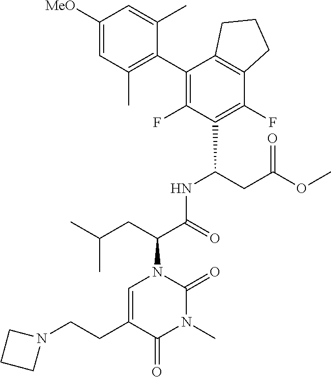 [SMILES: COC(=O)C[C@H](NC(=O)[C@H](CC(C)C)n1cc(CCN2CCC2)c(=O)n(C)c1=O)c1c(F)c2c(c(-c3c(C)cc(OC)cc3C)c1F)CCC2] |
| | 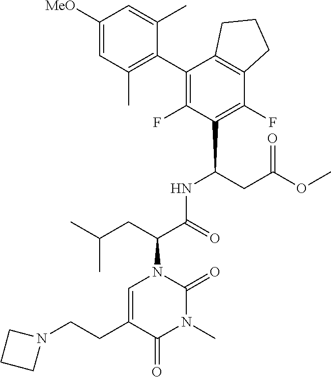 [SMILES: COC(=O)C[C@@H](NC(=O)[C@H](CC(C)C)n1cc(CCN2CCC2)c(=O)n(C)c1=O)c1c(F)c2c(c(-c3c(C)cc(OC)cc3C)c1F)CCC2] |
| | 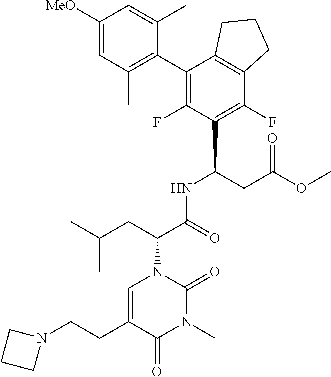 [SMILES: COC(=O)C[C@@H](NC(=O)[C@@H](CC(C)C)n1cc(CCN2CCC2)c(=O)n(C)c1=O)c1c(F)c2c(c(-c3c(C)cc(OC)cc3C)c1F)CCC2] |
| 337 | 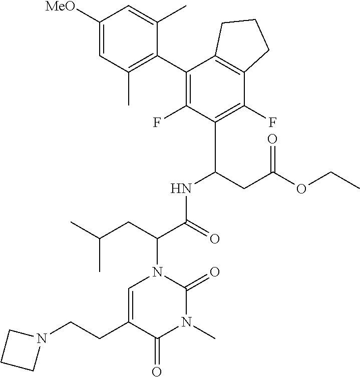 [SMILES: CCOC(=O)CC(NC(=O)C(CC(C)C)n1cc(CCN2CCC2)c(=O)n(C)c1=O)c1c(F)c2c(c(-c3c(C)cc(OC)cc3C)c1F)CCC2] |
| | 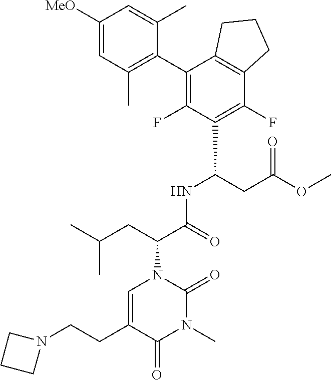 [SMILES: COC(=O)C[C@H](NC(=O)[C@@H](CC(C)C)n1cc(CCN2CCC2)c(=O)n(C)c1=O)c1c(F)c2c(c(-c3c(C)cc(OC)cc3C)c1F)CCC2] |
| | 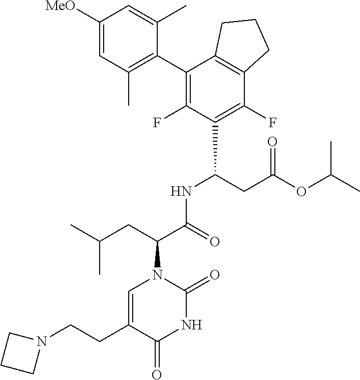 [SMILES: COc1cc(C)c(-c2c(F)c([C@H](CC(=O)OC(C)C)NC(=O)[C@H](CC(C)C)n3cc(CCN4CCC4)c(=O)[nH]c3=O)c(F)c3c2CCC3)c(C)c1] |
| | 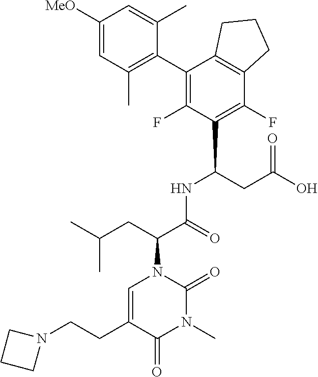 [SMILES: COc1cc(C)c(-c2c(F)c([C@@H](CC(=O)O)NC(=O)[C@H](CC(C)C)n3cc(CCN4CCC4)c(=O)n(C)c3=O)c(F)c3c2CCC3)c(C)c1] |
| | 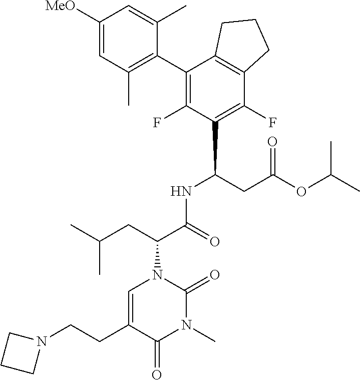 [SMILES: COc1cc(C)c(-c2c(F)c([C@@H](CC(=O)OC(C)C)NC(=O)[C@@H](CC(C)C)n3cc(CCN4CCC4)c(=O)n(C)c3=O)c(F)c3c2CCC3)c(C)c1] |
| 338 | 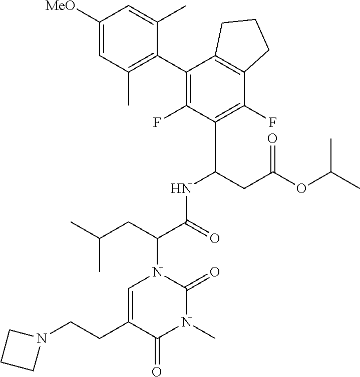 [SMILES: COc1cc(C)c(-c2c(F)c(C(CC(=O)OC(C)C)NC(=O)C(CC(C)C)n3cc(CCN4CCC4)c(=O)n(C)c3=O)c(F)c3c2CCC3)c(C)c1] |
| | 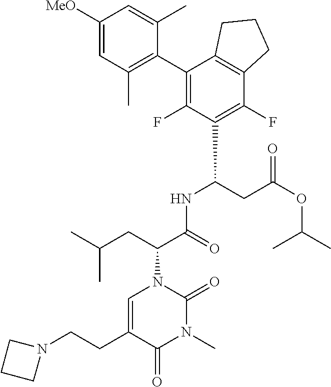 [SMILES: COc1cc(C)c(-c2c(F)c([C@H](CC(=O)OC(C)C)NC(=O)[C@@H](CC(C)C)n3cc(CCN4CCC4)c(=O)n(C)c3=O)c(F)c3c2CCC3)c(C)c1] |
| | 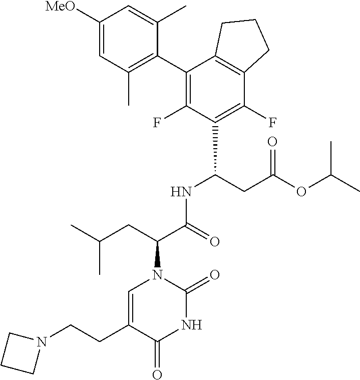 [SMILES: COc1cc(C)c(-c2c(F)c([C@H](CC(=O)OC(C)C)NC(=O)[C@H](CC(C)C)n3cc(CCN4CCC4)c(=O)[nH]c3=O)c(F)c3c2CCC3)c(C)c1] |
| | 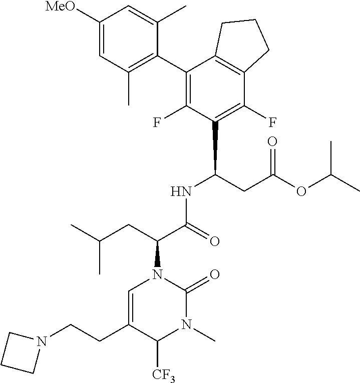 [SMILES: COc1cc(C)c(-c2c(F)c([C@@H](CC(=O)OC(C)C)NC(=O)[C@H](CC(C)C)N3C=C(CCN4CCC4)C(C(F)(F)F)N(C)C3=O)c(F)c3c2CCC3)c(C)c1] |
| | 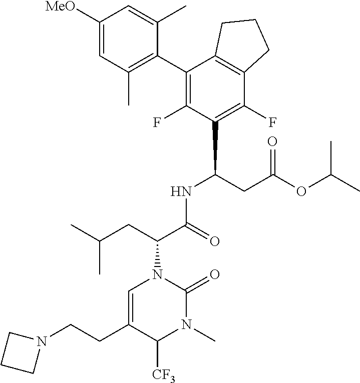 [SMILES: COc1cc(C)c(-c2c(F)c([C@@H](CC(=O)OC(C)C)NC(=O)[C@@H](CC(C)C)N3C=C(CCN4CCC4)C(C(F)(F)F)N(C)C3=O)c(F)c3c2CCC3)c(C)c1] |
| 339 | 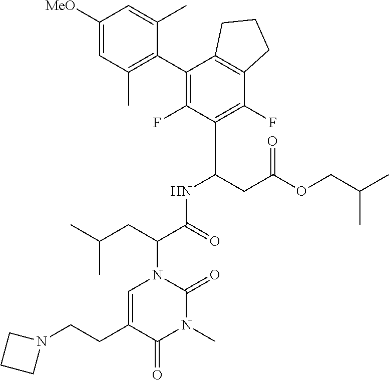 [SMILES: COc1cc(C)c(-c2c(F)c(C(CC(=O)OCC(C)C)NC(=O)C(CC(C)C)n3cc(CCN4CCC4)c(=O)n(C)c3=O)c(F)c3c2CCC3)c(C)c1] |
| | 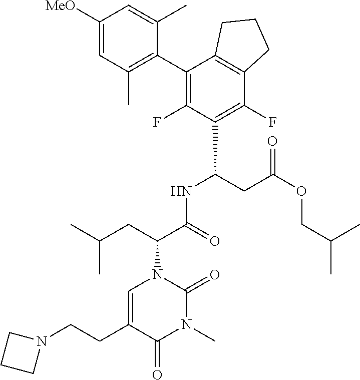 [SMILES: COc1cc(C)c(-c2c(F)c([C@H](CC(=O)OCC(C)C)NC(=O)[C@@H](CC(C)C)n3cc(CCN4CCC4)c(=O)n(C)c3=O)c(F)c3c2CCC3)c(C)c1] |
| | 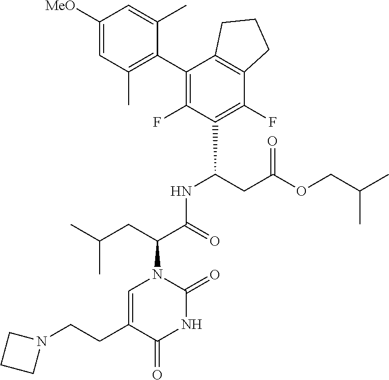 [SMILES: COc1cc(C)c(-c2c(F)c([C@H](CC(=O)OCC(C)C)NC(=O)[C@H](CC(C)C)n3cc(CCN4CCC4)c(=O)[nH]c3=O)c(F)c3c2CCC3)c(C)c1] |
| | 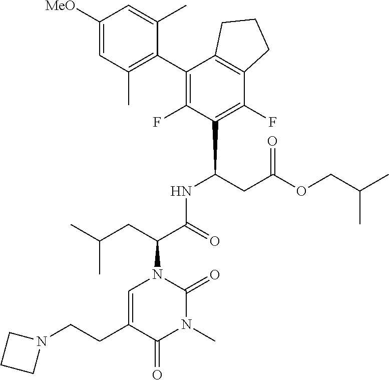 [SMILES: COc1cc(C)c(-c2c(F)c([C@@H](CC(=O)OCC(C)C)NC(=O)[C@H](CC(C)C)n3cc(CCN4CCC4)c(=O)n(C)c3=O)c(F)c3c2CCC3)c(C)c1] |
| | 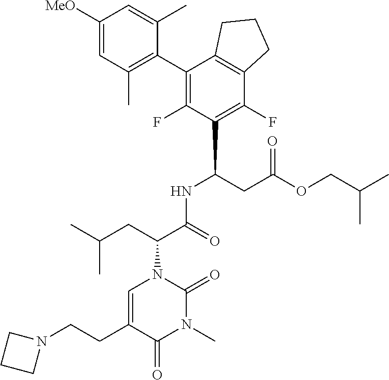 [SMILES: COc1cc(C)c(-c2c(F)c([C@@H](CC(=O)OCC(C)C)NC(=O)[C@@H](CC(C)C)n3cc(CCN4CCC4)c(=O)n(C)c3=O)c(F)c3c2CCC3)c(C)c1] |
| 340 | 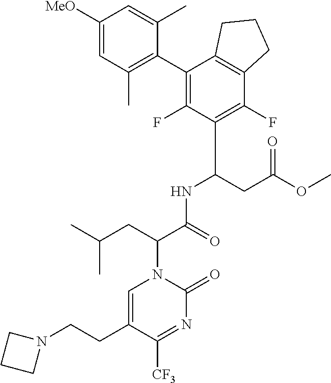 [SMILES: COC(=O)CC(NC(=O)C(CC(C)C)n1cc(CCN2CCC2)c(C(F)(F)F)nc1=O)c1c(F)c2c(c(-c3c(C)cc(OC)cc3C)c1F)CCC2] |
| | 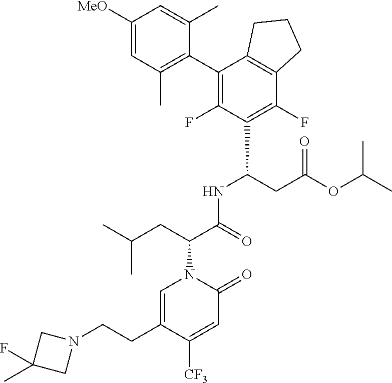 [SMILES: COc1cc(C)c(-c2c(F)c([C@H](CC(=O)OC(C)C)NC(=O)[C@@H](CC(C)C)n3cc(CCN4CC(C)(F)C4)c(C(F)(F)F)cc3=O)c(F)c3c2CCC3)c(C)c1] |
| | 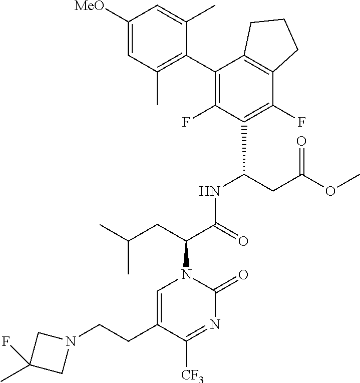 |
| | 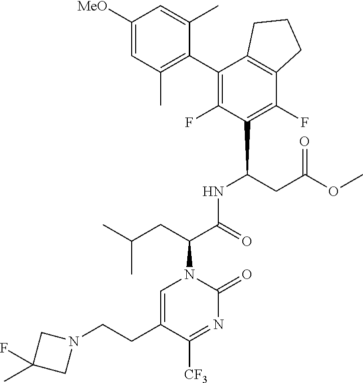 |
| | 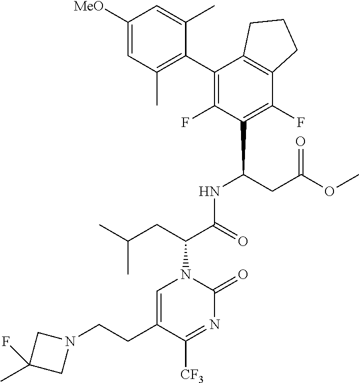 |
| 341 | 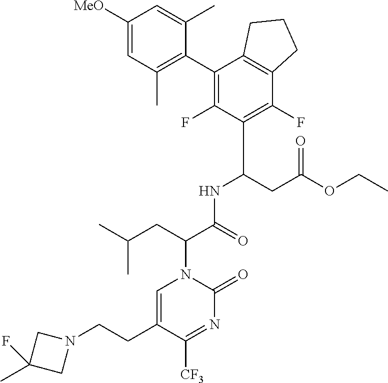 [SMILES: CCOC(=O)CC(NC(=O)C(CC(C)C)n1cc(CCN2CC(C)(F)C2)c(C(F)(F)F)nc1=O)c1c(F)c2c(c(-c3c(C)cc(OC)cc3C)c1F)CCC2] |
| | 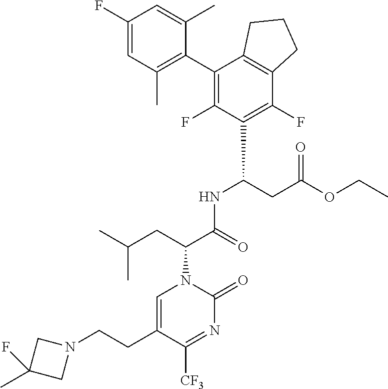 [SMILES: CCOC(=O)C[C@H](NC(=O)[C@@H](CC(C)C)n1cc(CCN2CC(C)(F)C2)c(C(F)(F)F)nc1=O)c1c(F)c2c(c(-c3c(C)cc(F)cc3C)c1F)CCC2] |
| | 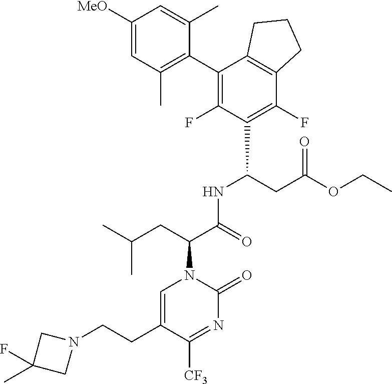 [SMILES: CCOC(=O)C[C@H](NC(=O)[C@H](CC(C)C)n1cc(CCN2CC(C)(F)C2)c(C(F)(F)F)nc1=O)c1c(F)c2c(c(-c3c(C)cc(OC)cc3C)c1F)CCC2] |
| | 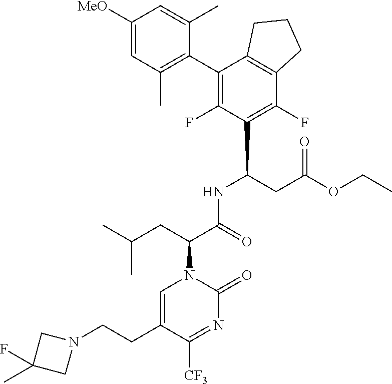 [SMILES: CCOC(=O)C[C@@H](NC(=O)[C@H](CC(C)C)n1cc(CCN2CC(C)(F)C2)c(C(F)(F)F)nc1=O)c1c(F)c2c(c(-c3c(C)cc(OC)cc3C)c1F)CCC2] |
| | 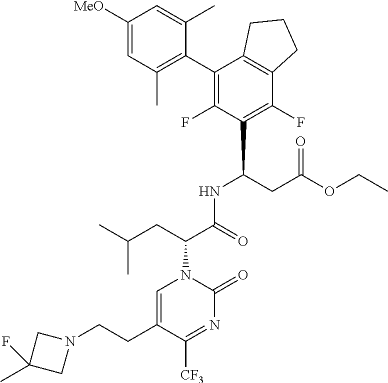 [SMILES: CCOC(=O)C[C@@H](NC(=O)[C@@H](CC(C)C)n1cc(CCN2CC(C)(F)C2)c(C(F)(F)F)nc1=O)c1c(F)c2c(c(-c3c(C)cc(OC)cc3C)c1F)CCC2] |
| 342 | 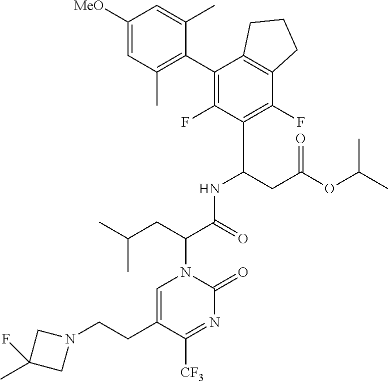 [SMILES: COc1cc(C)c(-c2c(F)c(C(CC(=O)OC(C)C)NC(=O)C(CC(C)C)n3cc(CCN4CC(C)(F)C4)c(C(F)(F)F)nc3=O)c(F)c3c2CCC3)c(C)c1] |
| | 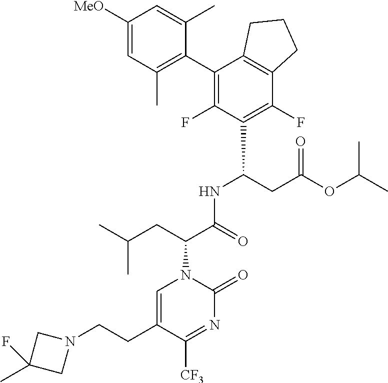 [SMILES: COc1cc(C)c(-c2c(F)c([C@H](CC(=O)OC(C)C)NC(=O)[C@@H](CC(C)C)n3cc(CCN4CC(C)(F)C4)c(C(F)(F)F)nc3=O)c(F)c3c2CCC3)c(C)c1] |
| | 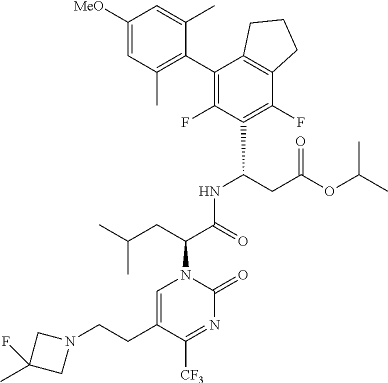 [SMILES: COc1cc(C)c(-c2c(F)c([C@H](CC(=O)OC(C)C)NC(=O)[C@H](CC(C)C)n3cc(CCN4CC(C)(F)C4)c(C(F)(F)F)nc3=O)c(F)c3c2CCC3)c(C)c1] |
| | 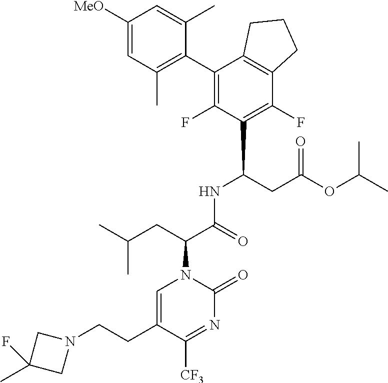 [SMILES: COc1cc(C)c(-c2c(F)c([C@@H](CC(=O)OC(C)C)NC(=O)[C@H](CC(C)C)n3cc(CCN4CC(C)(F)C4)c(C(F)(F)F)nc3=O)c(F)c3c2CCC3)c(C)c1] |
| | 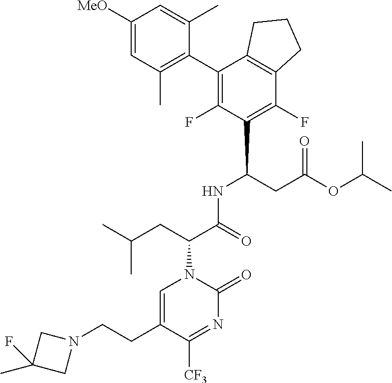 [SMILES: COc1cc(C)c(-c2c(F)c([C@@H](CC(=O)OC(C)C)NC(=O)[C@@H](CC(C)C)n3cc(CCN4CC(C)(F)C4)c(C(F)(F)F)nc3=O)c(F)c3c2CCC3)c(C)c1] |
| 343 | 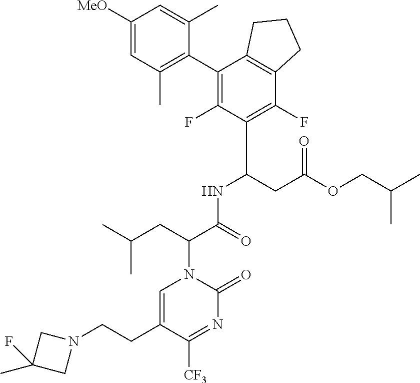 [SMILES: COc1cc(C)c(-c2c(F)c(C(CC(=O)OCC(C)C)NC(=O)C(CC(C)C)n3cc(CCN4CC(C)(F)C4)c(C(F)(F)F)nc3=O)c(F)c3c2CCC3)c(C)c1] |
| | 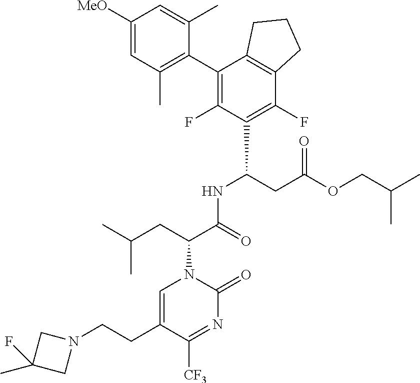 [SMILES: COc1cc(C)c(-c2c(F)c([C@H](CC(=O)OCC(C)C)NC(=O)[C@@H](CC(C)C)n3cc(CCN4CC(C)(F)C4)c(C(F)(F)F)nc3=O)c(F)c3c2CCC3)c(C)c1] |
| | 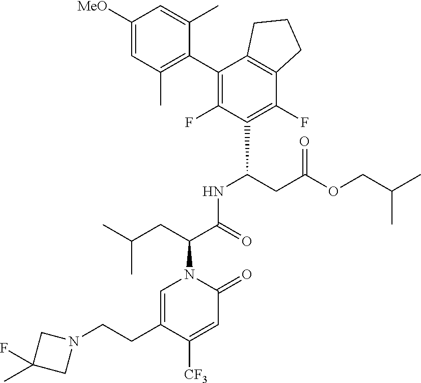 [SMILES: COc1cc(C)c(-c2c(F)c([C@H](CC(=O)OCC(C)C)NC(=O)[C@H](CC(C)C)n3cc(CCN4CC(C)(F)C4)c(C(F)(F)F)cc3=O)c(F)c3c2CCC3)c(C)c1] |
| | 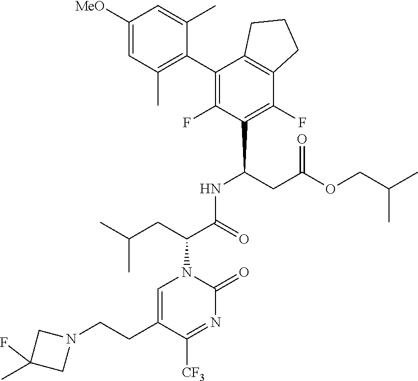 [SMILES: COc1cc(C)c(-c2c(F)c([C@@H](CC(=O)OCC(C)C)NC(=O)[C@@H](CC(C)C)n3cc(CCN4CC(C)(F)C4)c(C(F)(F)F)nc3=O)c(F)c3c2CCC3)c(C)c1] |
| | 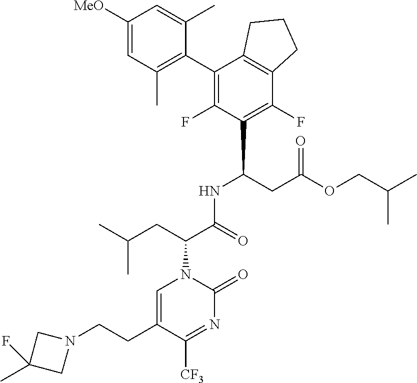 [SMILES: COc1cc(C)c(-c2c(F)c([C@@H](CC(=O)OCC(C)C)NC(=O)[C@@H](CC(C)C)n3cc(CCN4CC(C)(F)C4)c(C(F)(F)F)nc3=O)c(F)c3c2CCC3)c(C)c1] |
| 344 | 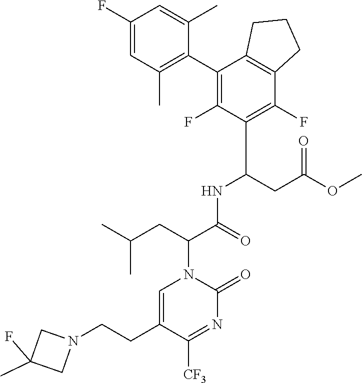 |
| | 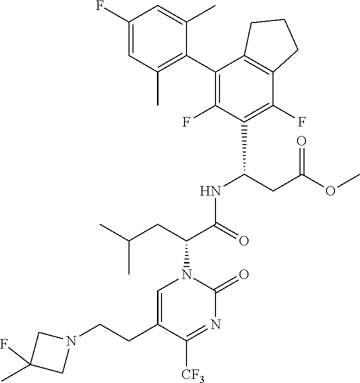 |
| | 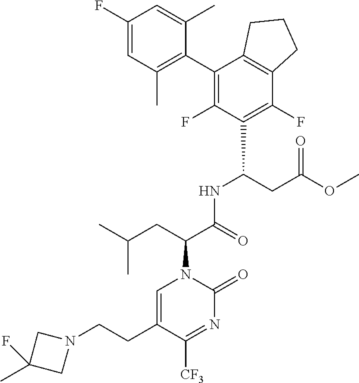 |
| | 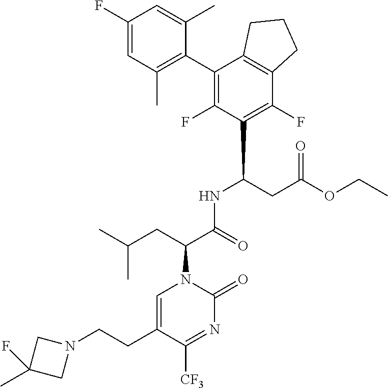 [SMILES: CCOC(=O)C[C@@H](NC(=O)[C@H](CC(C)C)n1cc(CCN2CC(C)(F)C2)c(C(F)(F)F)nc1=O)c1c(F)c2c(c(-c3c(C)cc(F)cc3C)c1F)CCC2] |
| | 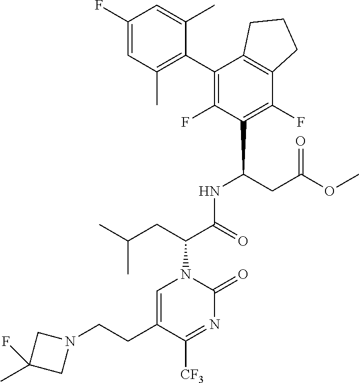 |
| 345 | 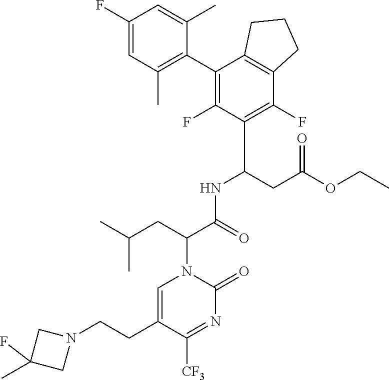 [SMILES: CCOC(=O)CC(NC(=O)C(CC(C)C)n1cc(CCN2CC(C)(F)C2)c(C(F)(F)F)nc1=O)c1c(F)c2c(c(-c3c(C)cc(F)cc3C)c1F)CCC2] |
| | 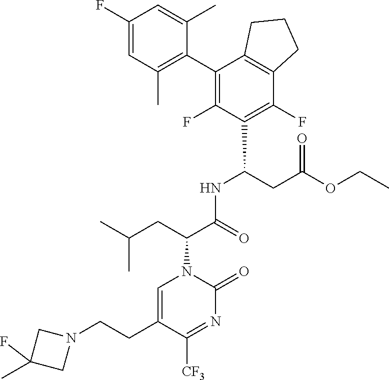 [SMILES: CCOC(=O)C[C@H](NC(=O)[C@@H](CC(C)C)n1cc(CCN2CC(C)(F)C2)c(C(F)(F)F)nc1=O)c1c(F)c2c(c(-c3c(C)cc(F)cc3C)c1F)CCC2] |
| | 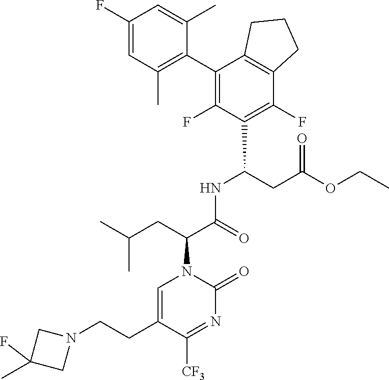 [SMILES: CCOC(=O)C[C@H](NC(=O)[C@H](CC(C)C)n1cc(CCN2CC(C)(F)C2)c(C(F)(F)F)nc1=O)c1c(F)c2c(c(-c3c(C)cc(F)cc3C)c1F)CCC2] |
| | 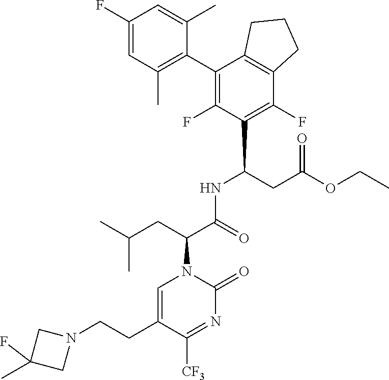 [SMILES: CCOC(=O)C[C@@H](NC(=O)[C@H](CC(C)C)n1cc(CCN2CC(C)(F)C2)c(C(F)(F)F)nc1=O)c1c(F)c2c(c(-c3c(C)cc(F)cc3C)c1F)CCC2] |
| | 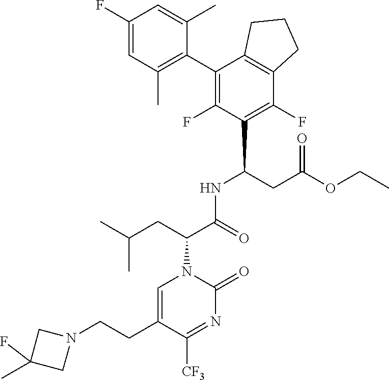 [SMILES: CCOC(=O)C[C@@H](NC(=O)[C@@H](CC(C)C)n1cc(CCN2CC(C)(F)C2)c(C(F)(F)F)nc1=O)c1c(F)c2c(c(-c3c(C)cc(F)cc3C)c1F)CCC2] |
| 346 | 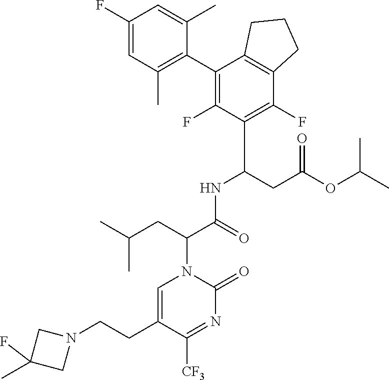 [SMILES: Cc1cc(F)cc(C)c1-c1c(F)c(C(CC(=O)OC(C)C)NC(=O)C(CC(C)C)n2cc(CCN3CC(C)(F)C3)c(C(F)(F)F)nc2=O)c(F)c2c1CCC2] |
| | 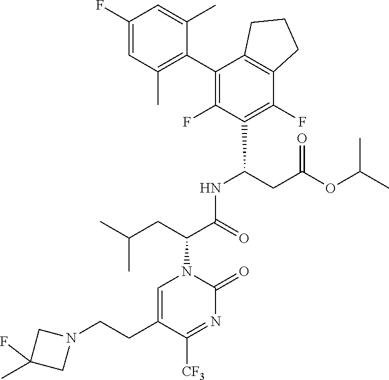 [SMILES: Cc1cc(F)cc(C)c1-c1c(F)c([C@H](CC(=O)OC(C)C)NC(=O)[C@@H](CC(C)C)n2cc(CCN3CC(C)(F)C3)c(C(F)(F)F)nc2=O)c(F)c2c1CCC2] |
| | 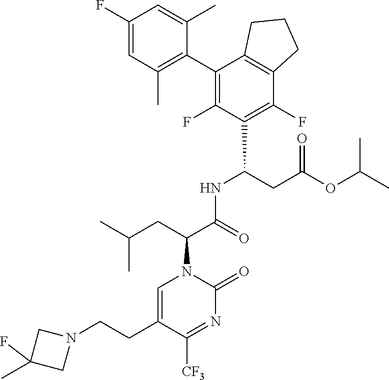 [SMILES: Cc1cc(F)cc(C)c1-c1c(F)c([C@H](CC(=O)OC(C)C)NC(=O)[C@H](CC(C)C)n2cc(CCN3CC(C)(F)C3)c(C(F)(F)F)nc2=O)c(F)c2c1CCC2] |
| | 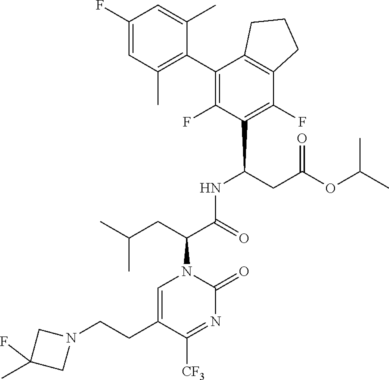 [SMILES: Cc1cc(F)cc(C)c1-c1c(F)c([C@@H](CC(=O)OC(C)C)NC(=O)[C@H](CC(C)C)n2cc(CCN3CC(C)(F)C3)c(C(F)(F)F)nc2=O)c(F)c2c1CCC2] |
| | 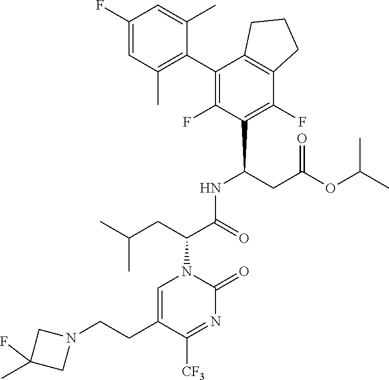 [SMILES: Cc1cc(F)cc(C)c1-c1c(F)c([C@@H](CC(=O)OC(C)C)NC(=O)[C@@H](CC(C)C)n2cc(CCN3CC(C)(F)C3)c(C(F)(F)F)nc2=O)c(F)c2c1CCC2] |
| 347 | 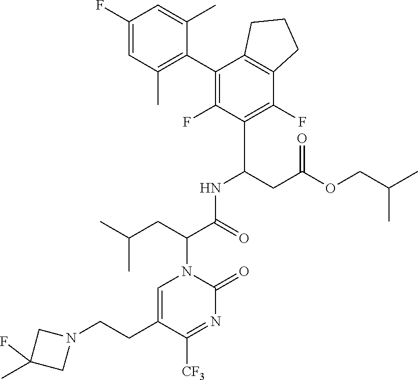 [SMILES: Cc1cc(F)cc(C)c1-c1c(F)c(C(CC(=O)OCC(C)C)NC(=O)C(CC(C)C)n2cc(CCN3CC(C)(F)C3)c(C(F)(F)F)nc2=O)c(F)c2c1CCC2] |
| | 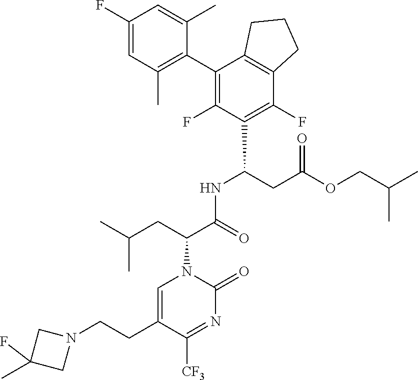 [SMILES: Cc1cc(F)cc(C)c1-c1c(F)c([C@H](CC(=O)OCC(C)C)NC(=O)[C@@H](CC(C)C)n2cc(CCN3CC(C)(F)C3)c(C(F)(F)F)nc2=O)c(F)c2c1CCC2] |
| | 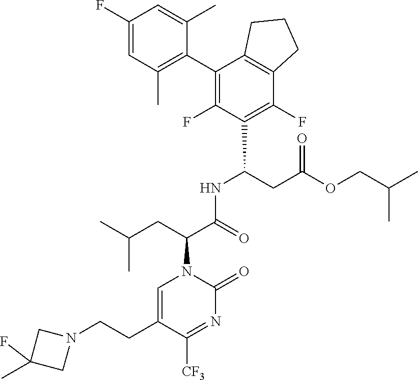 [SMILES: Cc1cc(F)cc(C)c1-c1c(F)c([C@H](CC(=O)OCC(C)C)NC(=O)[C@H](CC(C)C)n2cc(CCN3CC(C)(F)C3)c(C(F)(F)F)nc2=O)c(F)c2c1CCC2] |
| | 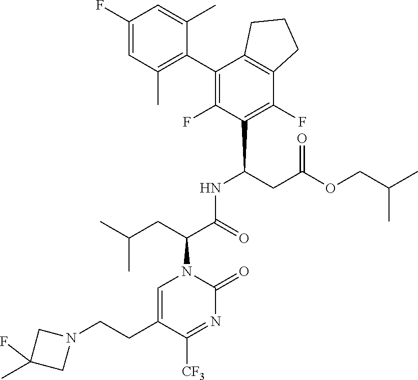 [SMILES: Cc1cc(F)cc(C)c1-c1c(F)c([C@@H](CC(=O)OCC(C)C)NC(=O)[C@H](CC(C)C)n2cc(CCN3CC(C)(F)C3)c(C(F)(F)F)nc2=O)c(F)c2c1CCC2] |
| | 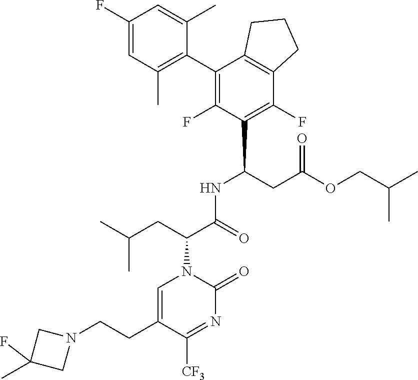 [SMILES: Cc1cc(F)cc(C)c1-c1c(F)c([C@@H](CC(=O)OCC(C)C)NC(=O)[C@@H](CC(C)C)n2cc(CCN3CC(C)(F)C3)c(C(F)(F)F)nc2=O)c(F)c2c1CCC2] |
| 348 | 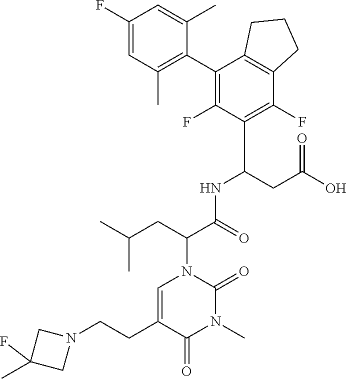 [SMILES: Cc1cc(F)cc(C)c1-c1c(F)c(C(CC(=O)O)NC(=O)C(CC(C)C)n2cc(CCN3CC(C)(F)C3)c(=O)n(C)c2=O)c(F)c2c1CCC2] |
| | 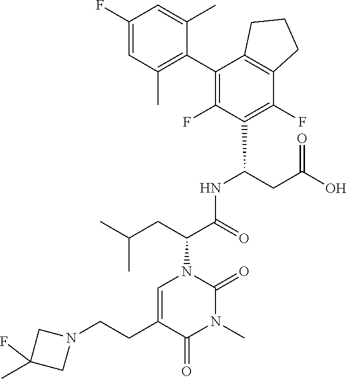 [SMILES: Cc1cc(F)cc(C)c1-c1c(F)c([C@H](CC(=O)O)NC(=O)[C@@H](CC(C)C)n2cc(CCN3CC(C)(F)C3)c(=O)n(C)c2=O)c(F)c2c1CCC2] |
| | 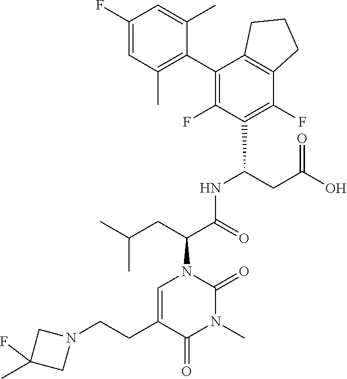 [SMILES: Cc1cc(F)cc(C)c1-c1c(F)c([C@H](CC(=O)O)NC(=O)[C@H](CC(C)C)n2cc(CCN3CC(C)(F)C3)c(=O)n(C)c2=O)c(F)c2c1CCC2] |
| | 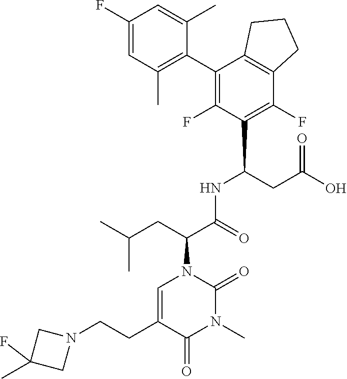 [SMILES: Cc1cc(F)cc(C)c1-c1c(F)c([C@@H](CC(=O)O)NC(=O)[C@H](CC(C)C)n2cc(CCN3CC(C)(F)C3)c(=O)n(C)c2=O)c(F)c2c1CCC2] |
| | 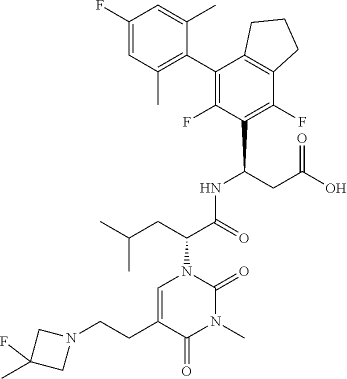 [SMILES: Cc1cc(F)cc(C)c1-c1c(F)c([C@@H](CC(=O)O)NC(=O)[C@@H](CC(C)C)n2cc(CCN3CC(C)(F)C3)c(=O)n(C)c2=O)c(F)c2c1CCC2] |
| 349 | 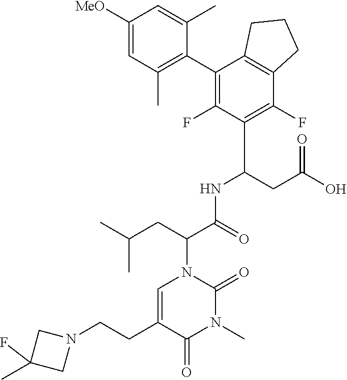 [SMILES: COc1cc(C)c(-c2c(F)c(C(CC(=O)O)NC(=O)C(CC(C)C)n3cc(CCN4CC(C)(F)C4)c(=O)n(C)c3=O)c(F)c3c2CCC3)c(C)c1] |
| | 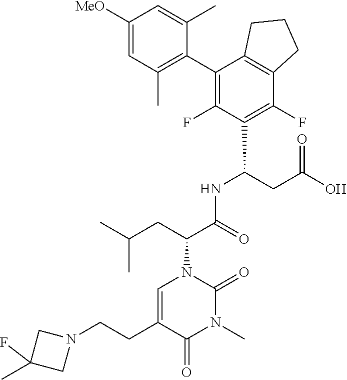 [SMILES: COc1cc(C)c(-c2c(F)c([C@H](CC(=O)O)NC(=O)[C@@H](CC(C)C)n3cc(CCN4CC(C)(F)C4)c(=O)n(C)c3=O)c(F)c3c2CCC3)c(C)c1] |
| | 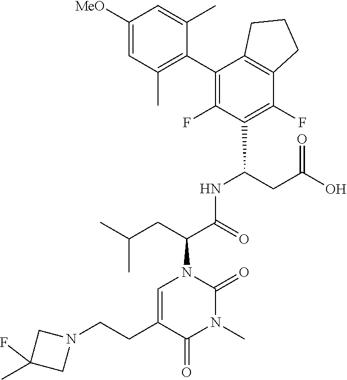 [SMILES: COc1cc(C)c(-c2c(F)c([C@H](CC(=O)O)NC(=O)[C@H](CC(C)C)n3cc(CCN4CC(C)(F)C4)c(=O)n(C)c3=O)c(F)c3c2CCC3)c(C)c1] |
| | 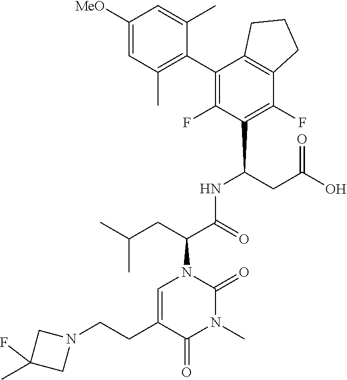 [SMILES: COc1cc(C)c(-c2c(F)c([C@@H](CC(=O)O)NC(=O)[C@H](CC(C)C)n3cc(CCN4CC(C)(F)C4)c(=O)n(C)c3=O)c(F)c3c2CCC3)c(C)c1] |
| | 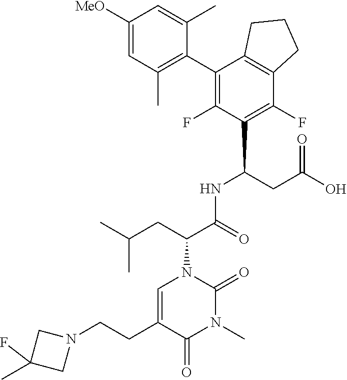 [SMILES: COc1cc(C)c(-c2c(F)c([C@@H](CC(=O)O)NC(=O)[C@@H](CC(C)C)n3cc(CCN4CC(C)(F)C4)c(=O)n(C)c3=O)c(F)c3c2CCC3)c(C)c1] |
| 350 | 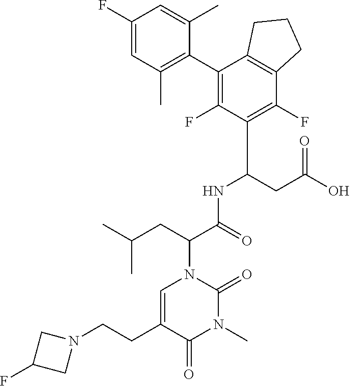 [SMILES: Cc1cc(F)cc(C)c1-c1c(F)c(C(CC(=O)O)NC(=O)C(CC(C)C)n2cc(CCN3CC(F)C3)c(=O)n(C)c2=O)c(F)c2c1CCC2] |
| | 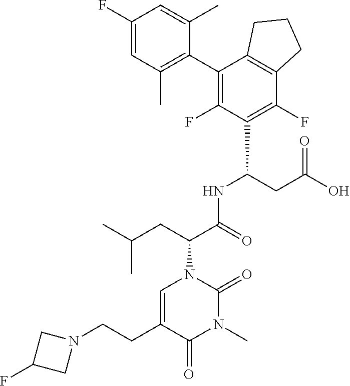 [SMILES: Cc1cc(F)cc(C)c1-c1c(F)c([C@H](CC(=O)O)NC(=O)[C@@H](CC(C)C)n2cc(CCN3CC(F)C3)c(=O)n(C)c2=O)c(F)c2c1CCC2] |
| | 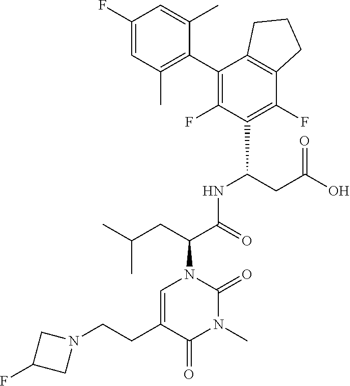 [SMILES: Cc1cc(F)cc(C)c1-c1c(F)c([C@H](CC(=O)O)NC(=O)[C@H](CC(C)C)n2cc(CCN3CC(F)C3)c(=O)n(C)c2=O)c(F)c2c1CCC2] |
| | 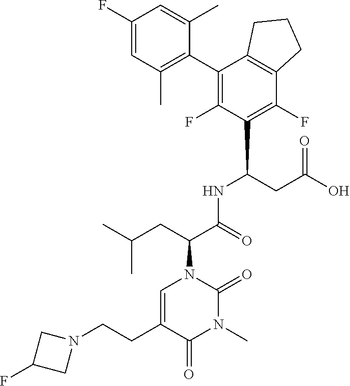 [SMILES: Cc1cc(F)cc(C)c1-c1c(F)c([C@@H](CC(=O)O)NC(=O)[C@H](CC(C)C)n2cc(CCN3CC(F)C3)c(=O)n(C)c2=O)c(F)c2c1CCC2] |
| | 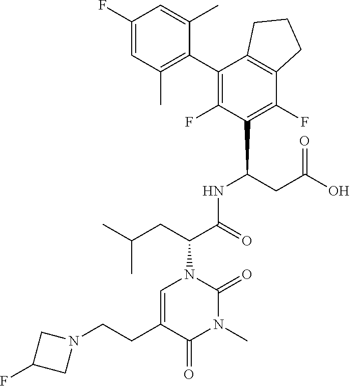 [SMILES: Cc1cc(F)cc(C)c1-c1c(F)c([C@@H](CC(=O)O)NC(=O)[C@@H](CC(C)C)n2cc(CCN3CC(F)C3)c(=O)n(C)c2=O)c(F)c2c1CCC2] |
| 351 | 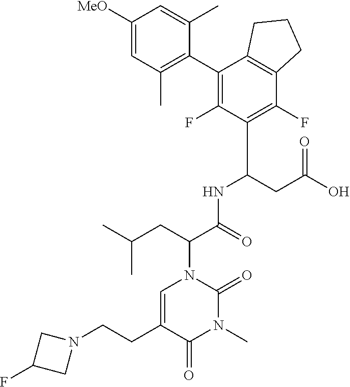 [SMILES: COc1cc(C)c(-c2c(F)c(C(CC(=O)O)NC(=O)C(CC(C)C)n3cc(CCN4CC(F)C4)c(=O)n(C)c3=O)c(F)c3c2CCC3)c(C)c1] |
| | 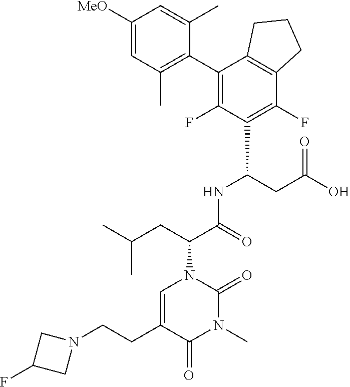 [SMILES: COc1cc(C)c(-c2c(F)c([C@H](CC(=O)O)NC(=O)[C@@H](CC(C)C)n3cc(CCN4CC(F)C4)c(=O)n(C)c3=O)c(F)c3c2CCC3)c(C)c1] |
| | 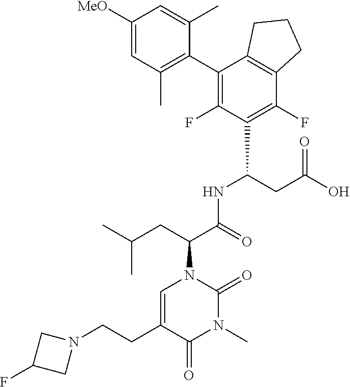 [SMILES: COc1cc(C)c(-c2c(F)c([C@H](CC(=O)O)NC(=O)[C@H](CC(C)C)n3cc(CCN4CC(F)C4)c(=O)n(C)c3=O)c(F)c3c2CCC3)c(C)c1] |
| | 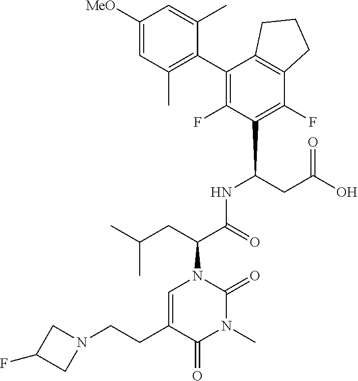 [SMILES: COc1cc(C)c(-c2c(F)c([C@@H](CC(=O)O)NC(=O)[C@H](CC(C)C)n3cc(CCN4CC(F)C4)c(=O)n(C)c3=O)c(F)c3c2CCC3)c(C)c1] |
| | 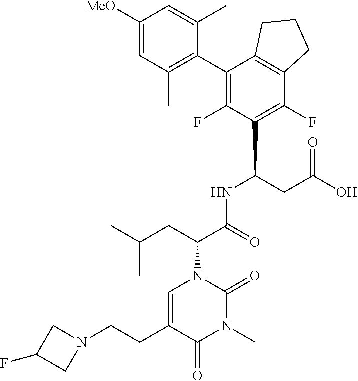 [SMILES: COc1cc(C)c(-c2c(F)c([C@@H](CC(=O)O)NC(=O)[C@@H](CC(C)C)n3cc(CCN4CC(F)C4)c(=O)n(C)c3=O)c(F)c3c2CCC3)c(C)c1] |
| 352 | 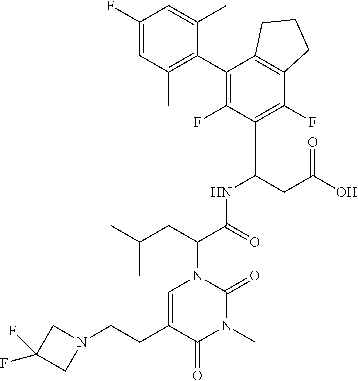 [SMILES: Cc1cc(F)cc(C)c1-c1c(F)c(C(CC(=O)O)NC(=O)C(CC(C)C)n2cc(CCN3CC(F)(F)C3)c(=O)n(C)c2=O)c(F)c2c1CCC2] |
| | 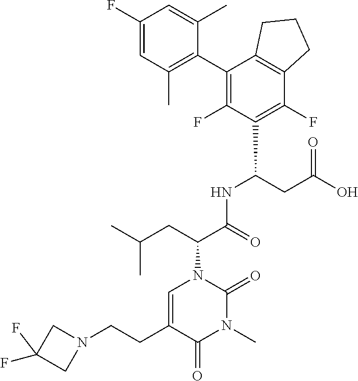 [SMILES: Cc1cc(F)cc(C)c1-c1c(F)c([C@H](CC(=O)O)NC(=O)[C@@H](CC(C)C)n2cc(CCN3CC(F)(F)C3)c(=O)n(C)c2=O)c(F)c2c1CCC2] |
| | 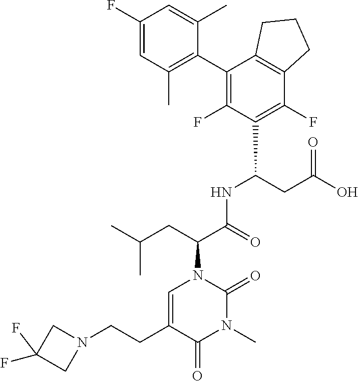 [SMILES: Cc1cc(F)cc(C)c1-c1c(F)c([C@H](CC(=O)O)NC(=O)[C@H](CC(C)C)n2cc(CCN3CC(F)(F)C3)c(=O)n(C)c2=O)c(F)c2c1CCC2] |
| | 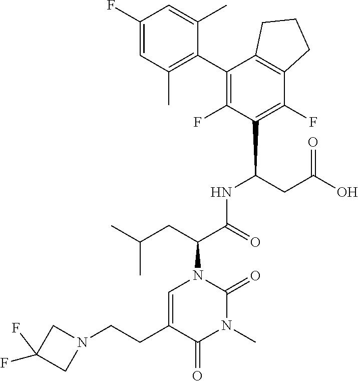 [SMILES: Cc1cc(F)cc(C)c1-c1c(F)c([C@@H](CC(=O)O)NC(=O)[C@H](CC(C)C)n2cc(CCN3CC(F)(F)C3)c(=O)n(C)c2=O)c(F)c2c1CCC2] |
| | 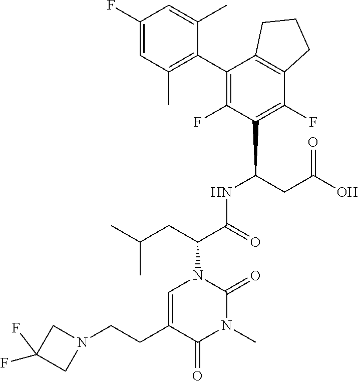 [SMILES: Cc1cc(F)cc(C)c1-c1c(F)c([C@@H](CC(=O)O)NC(=O)[C@@H](CC(C)C)n2cc(CCN3CC(F)(F)C3)c(=O)n(C)c2=O)c(F)c2c1CCC2] |
| 353 | 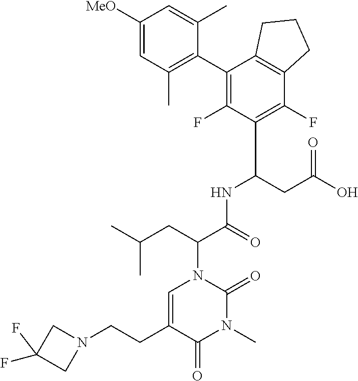 [SMILES: COc1cc(C)c(-c2c(F)c(C(CC(=O)O)NC(=O)C(CC(C)C)n3cc(CCN4CC(F)(F)C4)c(=O)n(C)c3=O)c(F)c3c2CCC3)c(C)c1] |
| | 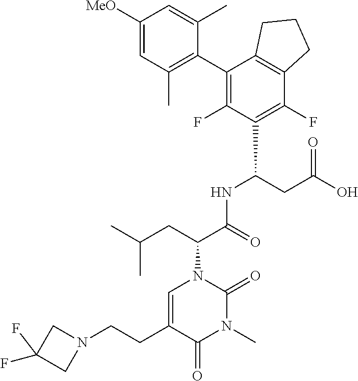 [SMILES: COc1cc(C)c(-c2c(F)c([C@H](CC(=O)O)NC(=O)[C@@H](CC(C)C)n3cc(CCN4CC(F)(F)C4)c(=O)n(C)c3=O)c(F)c3c2CCC3)c(C)c1] |
| | 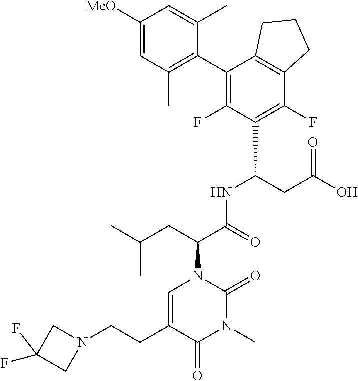 [SMILES: COc1cc(C)c(-c2c(F)c([C@H](CC(=O)O)NC(=O)[C@H](CC(C)C)n3cc(CCN4CC(F)(F)C4)c(=O)n(C)c3=O)c(F)c3c2CCC3)c(C)c1] |
| | 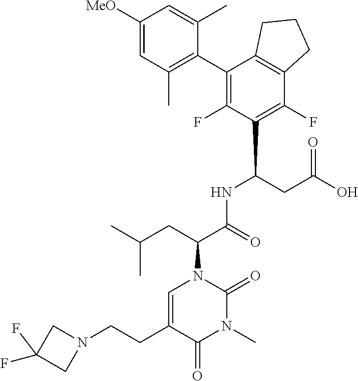 [SMILES: COc1cc(C)c(-c2c(F)c([C@@H](CC(=O)O)NC(=O)[C@H](CC(C)C)n3cc(CCN4CC(F)(F)C4)c(=O)n(C)c3=O)c(F)c3c2CCC3)c(C)c1] |
| | 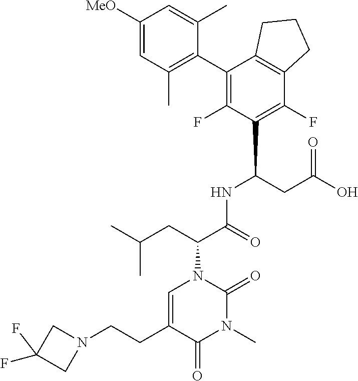 [SMILES: COc1cc(C)c(-c2c(F)c([C@@H](CC(=O)O)NC(=O)[C@@H](CC(C)C)n3cc(CCN4CC(F)(F)C4)c(=O)n(C)c3=O)c(F)c3c2CCC3)c(C)c1] |
| 354 | 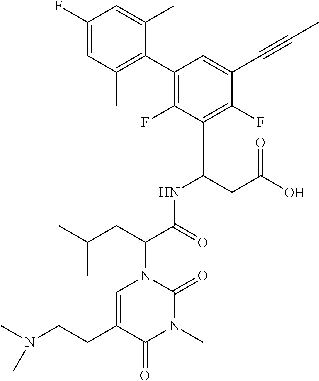 [SMILES: CC#Cc1cc(-c2c(C)cc(F)cc2C)c(F)c(C(CC(=O)O)NC(=O)C(CC(C)C)n2cc(CCN(C)C)c(=O)n(C)c2=O)c1F] |
| | 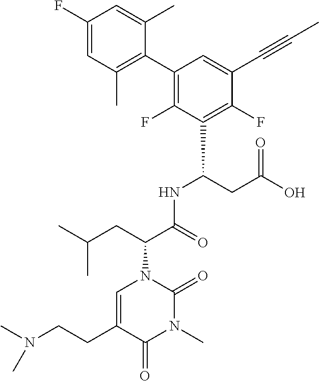 [SMILES: CC#Cc1cc(-c2c(C)cc(F)cc2C)c(F)c([C@H](CC(=O)O)NC(=O)[C@@H](CC(C)C)n2cc(CCN(C)C)c(=O)n(C)c2=O)c1F] |
| | 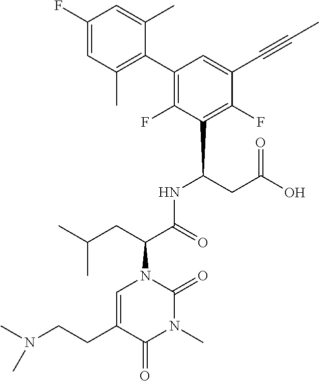 [SMILES: CC#Cc1cc(-c2c(C)cc(F)cc2C)c(F)c([C@@H](CC(=O)O)NC(=O)[C@H](CC(C)C)n2cc(CCN(C)C)c(=O)n(C)c2=O)c1F] |
| | 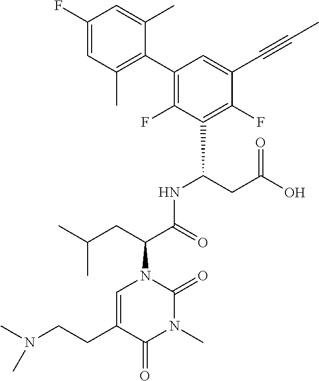 [SMILES: CC#Cc1cc(-c2c(C)cc(F)cc2C)c(F)c([C@H](CC(=O)O)NC(=O)[C@H](CC(C)C)n2cc(CCN(C)C)c(=O)n(C)c2=O)c1F] |
| | 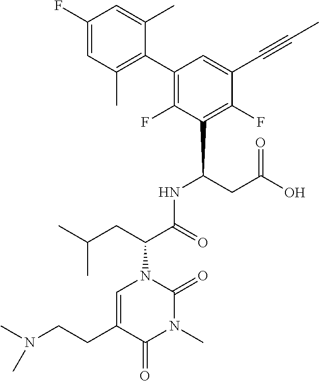 [SMILES: CC#Cc1cc(-c2c(C)cc(F)cc2C)c(F)c([C@@H](CC(=O)O)NC(=O)[C@@H](CC(C)C)n2cc(CCN(C)C)c(=O)n(C)c2=O)c1F] |
| 355 | 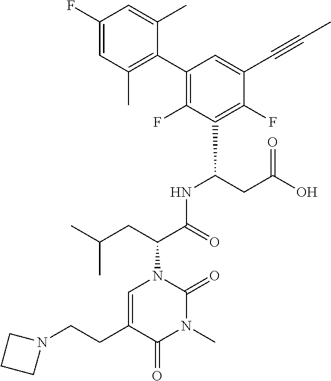 [SMILES: CC#Cc1cc(-c2c(C)cc(F)cc2C)c(F)c([C@H](CC(=O)O)NC(=O)[C@@H](CC(C)C)n2cc(CCN3CCC3)c(=O)n(C)c2=O)c1F] |
| | 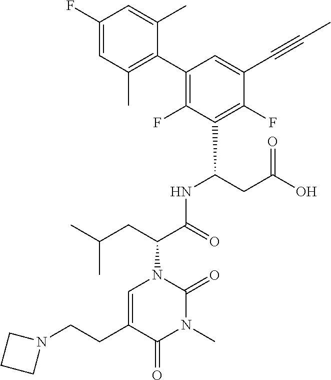 [SMILES: CC#Cc1cc(-c2c(C)cc(F)cc2C)c(F)c([C@H](CC(=O)O)NC(=O)[C@@H](CC(C)C)n2cc(CCN3CCC3)c(=O)n(C)c2=O)c1F] |
| | 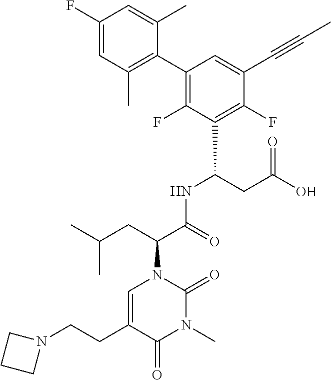 [SMILES: CC#Cc1cc(-c2c(C)cc(F)cc2C)c(F)c([C@H](CC(=O)O)NC(=O)[C@H](CC(C)C)n2cc(CCN3CCC3)c(=O)n(C)c2=O)c1F] |
| | 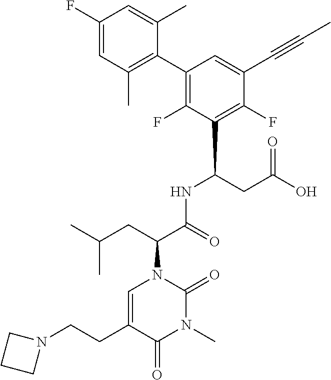 [SMILES: CC#Cc1cc(-c2c(C)cc(F)cc2C)c(F)c([C@@H](CC(=O)O)NC(=O)[C@H](CC(C)C)n2cc(CCN3CCC3)c(=O)n(C)c2=O)c1F] |
| | 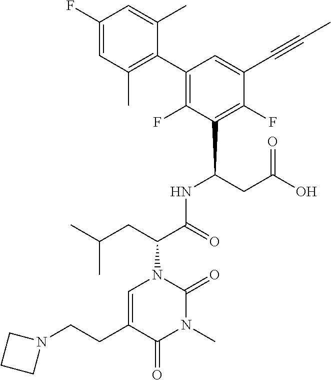 [SMILES: CC#Cc1cc(-c2c(C)cc(F)cc2C)c(F)c([C@@H](CC(=O)O)NC(=O)[C@@H](CC(C)C)n2cc(CCN3CCC3)c(=O)n(C)c2=O)c1F] |
| 356 | 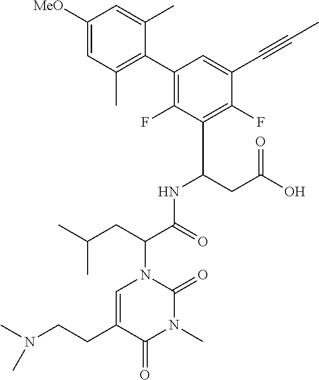 [SMILES: CC#Cc1cc(-c2c(C)cc(OC)cc2C)c(F)c(C(CC(=O)O)NC(=O)C(CC(C)C)n2cc(CCN(C)C)c(=O)n(C)c2=O)c1F] |
| | 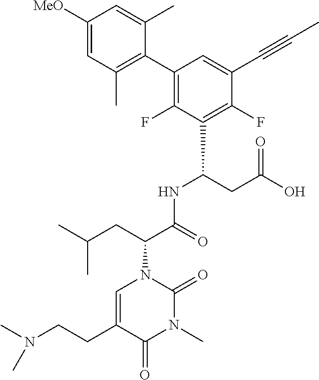 [SMILES: CC#Cc1cc(-c2c(C)cc(OC)cc2C)c(F)c([C@H](CC(=O)O)NC(=O)[C@@H](CC(C)C)n2cc(CCN(C)C)c(=O)n(C)c2=O)c1F] |
| | 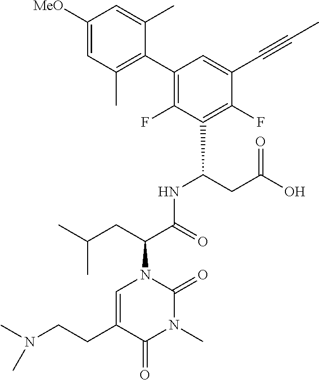 [SMILES: CC#Cc1cc(-c2c(C)cc(OC)cc2C)c(F)c([C@H](CC(=O)O)NC(=O)[C@H](CC(C)C)n2cc(CCN(C)C)c(=O)n(C)c2=O)c1F] |
| | 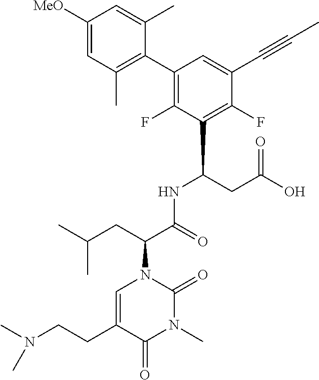 [SMILES: CC#Cc1cc(-c2c(C)cc(OC)cc2C)c(F)c([C@@H](CC(=O)O)NC(=O)[C@H](CC(C)C)n2cc(CCN(C)C)c(=O)n(C)c2=O)c1F] |
| | 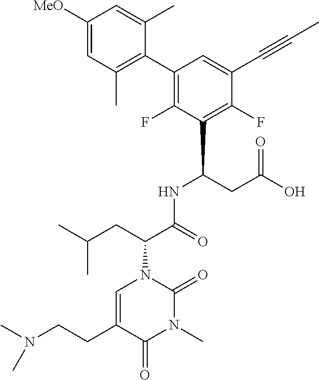 [SMILES: CC#Cc1cc(-c2c(C)cc(OC)cc2C)c(F)c([C@@H](CC(=O)O)NC(=O)[C@@H](CC(C)C)n2cc(CCN(C)C)c(=O)n(C)c2=O)c1F] |
| 357 | 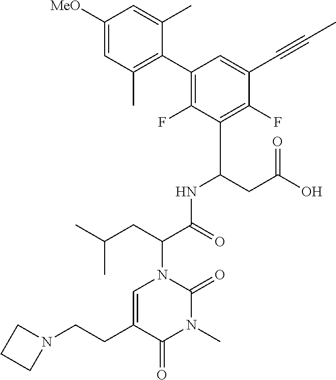 [SMILES: CC#Cc1cc(-c2c(C)cc(OC)cc2C)c(F)c(C(CC(=O)O)NC(=O)C(CC(C)C)n2cc(CCN3CCC3)c(=O)n(C)c2=O)c1F] |
| | 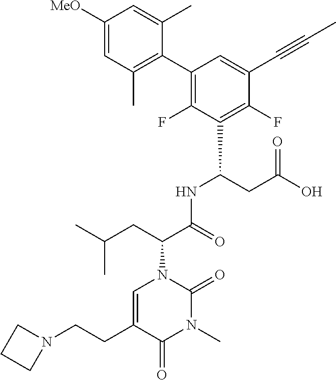 [SMILES: CC#Cc1cc(-c2c(C)cc(OC)cc2C)c(F)c([C@H](CC(=O)O)NC(=O)[C@@H](CC(C)C)n2cc(CCN3CCC3)c(=O)n(C)c2=O)c1F] |
| | 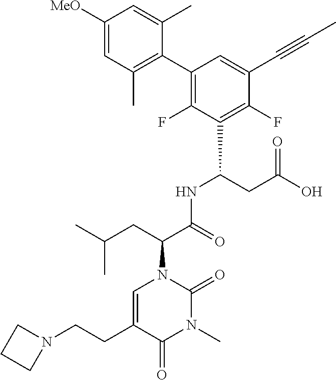 [SMILES: CC#Cc1cc(-c2c(C)cc(OC)cc2C)c(F)c([C@H](CC(=O)O)NC(=O)[C@H](CC(C)C)n2cc(CCN3CCC3)c(=O)n(C)c2=O)c1F] |
| | 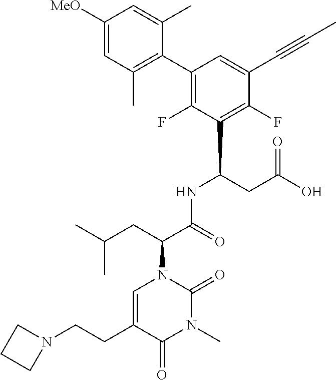 [SMILES: CC#Cc1cc(-c2c(C)cc(OC)cc2C)c(F)c([C@@H](CC(=O)O)NC(=O)[C@H](CC(C)C)n2cc(CCN3CCC3)c(=O)n(C)c2=O)c1F] |
| | 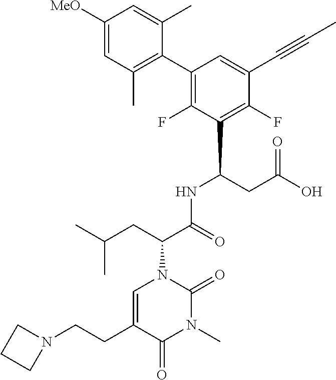 [SMILES: CC#Cc1cc(-c2c(C)cc(OC)cc2C)c(F)c([C@@H](CC(=O)O)NC(=O)[C@@H](CC(C)C)n2cc(CCN3CCC3)c(=O)n(C)c2=O)c1F] |
| 358 | 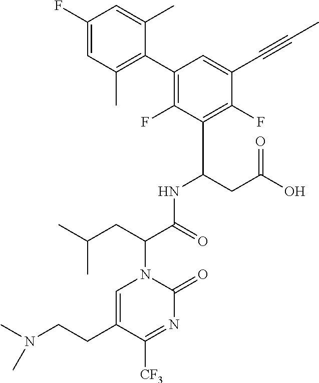 [SMILES: CC#Cc1cc(-c2c(C)cc(F)cc2C)c(F)c(C(CC(=O)O)NC(=O)C(CC(C)C)n2cc(CCN(C)C)c(C(F)(F)F)nc2=O)c1F] |
| | 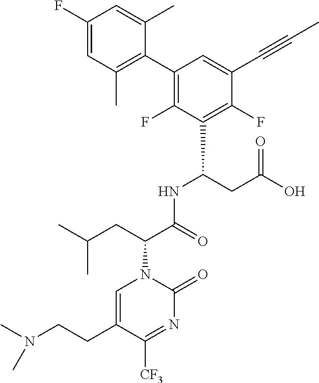 [SMILES: CC#Cc1cc(-c2c(C)cc(F)cc2C)c(F)c([C@H](CC(=O)O)NC(=O)[C@@H](CC(C)C)n2cc(CCN(C)C)c(C(F)(F)F)nc2=O)c1F] |
| | 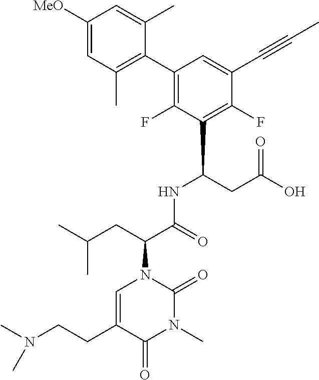 [SMILES: CC#Cc1cc(-c2c(C)cc(OC)cc2C)c(F)c([C@@H](CC(=O)O)NC(=O)[C@H](CC(C)C)n2cc(CCN(C)C)c(=O)n(C)c2=O)c1F] |
| | 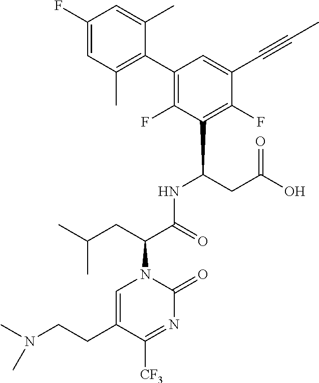 [SMILES: CC#Cc1cc(-c2c(C)cc(F)cc2C)c(F)c([C@@H](CC(=O)O)NC(=O)[C@H](CC(C)C)n2cc(CCN(C)C)c(C(F)(F)F)nc2=O)c1F] |
| | 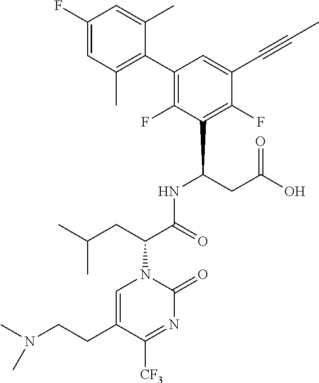 [SMILES: CC#Cc1cc(-c2c(C)cc(F)cc2C)c(F)c([C@@H](CC(=O)O)NC(=O)[C@@H](CC(C)C)n2cc(CCN(C)C)c(C(F)(F)F)nc2=O)c1F] |
| 359 | 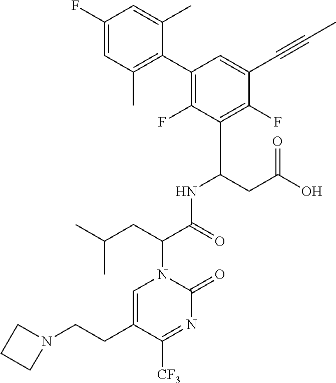 [SMILES: CC#Cc1cc(-c2c(C)cc(F)cc2C)c(F)c(C(CC(=O)O)NC(=O)C(CC(C)C)n2cc(CCN3CCC3)c(C(F)(F)F)nc2=O)c1F] |
| 360 | 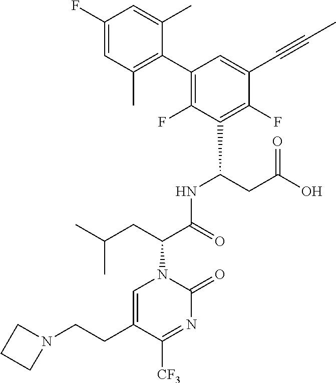 [SMILES: CC#Cc1cc(-c2c(C)cc(F)cc2C)c(F)c([C@H](CC(=O)O)NC(=O)[C@@H](CC(C)C)n2cc(CCN3CCC3)c(C(F)(F)F)nc2=O)c1F] |
| | 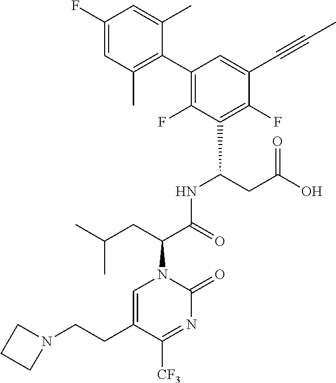 [SMILES: CC#Cc1cc(-c2c(C)cc(F)cc2C)c(F)c([C@H](CC(=O)O)NC(=O)[C@H](CC(C)C)n2cc(CCN3CCC3)c(C(F)(F)F)nc2=O)c1F] |
| | 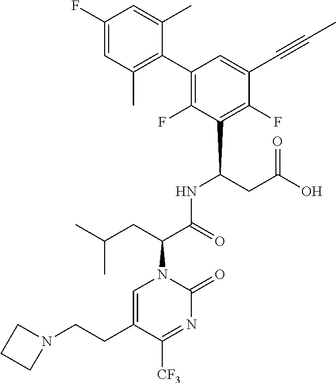 [SMILES: CC#Cc1cc(-c2c(C)cc(F)cc2C)c(F)c([C@@H](CC(=O)O)NC(=O)[C@H](CC(C)C)n2cc(CCN3CCC3)c(C(F)(F)F)nc2=O)c1F] |
| | 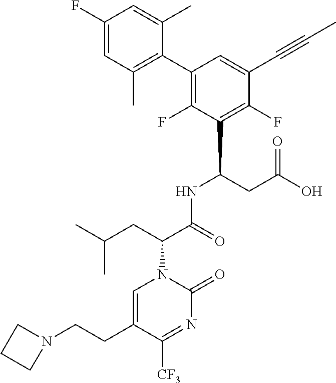 [SMILES: CC#Cc1cc(-c2c(C)cc(F)cc2C)c(F)c([C@@H](CC(=O)O)NC(=O)[C@@H](CC(C)C)n2cc(CCN3CCC3)c(C(F)(F)F)nc2=O)c1F] |
| 360 | 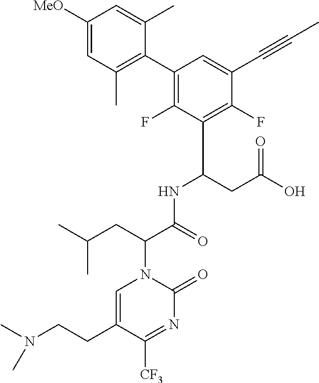 [SMILES: CC#Cc1cc(-c2c(C)cc(OC)cc2C)c(F)c(C(CC(=O)O)NC(=O)C(CC(C)C)n2cc(CCN(C)C)c(C(F)(F)F)nc2=O)c1F] |
| | 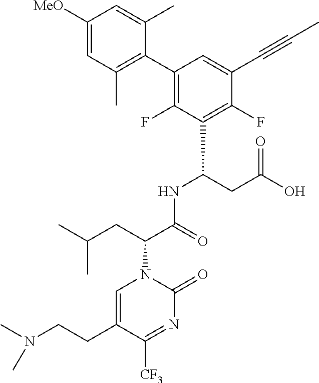 [SMILES: CC#Cc1cc(-c2c(C)cc(OC)cc2C)c(F)c([C@H](CC(=O)O)NC(=O)[C@@H](CC(C)C)n2cc(CCN(C)C)c(C(F)(F)F)nc2=O)c1F] |
| | 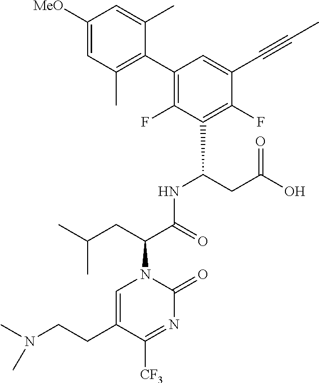 [SMILES: CC#Cc1cc(-c2c(C)cc(OC)cc2C)c(F)c([C@H](CC(=O)O)NC(=O)[C@H](CC(C)C)n2cc(CCN(C)C)c(C(F)(F)F)nc2=O)c1F] |
| | 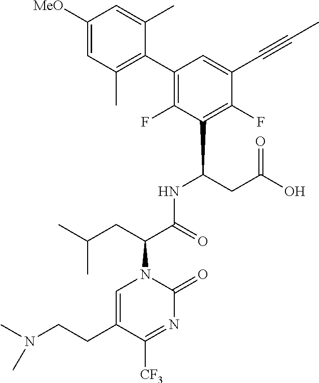 [SMILES: CC#Cc1cc(-c2c(C)cc(OC)cc2C)c(F)c([C@@H](CC(=O)O)NC(=O)[C@H](CC(C)C)n2cc(CCN(C)C)c(C(F)(F)F)nc2=O)c1F] |
| | 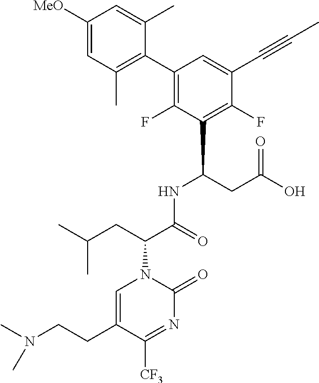 [SMILES: CC#Cc1cc(-c2c(C)cc(OC)cc2C)c(F)c([C@@H](CC(=O)O)NC(=O)[C@@H](CC(C)C)n2cc(CCN(C)C)c(C(F)(F)F)nc2=O)c1F] |
| 361 | 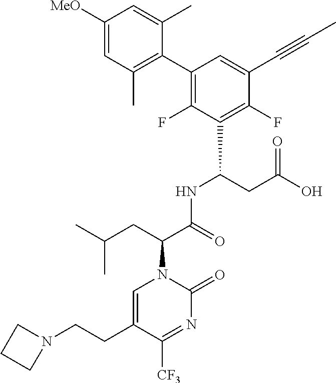 [SMILES: CC#Cc1cc(-c2c(C)cc(OC)cc2C)c(F)c([C@H](CC(=O)O)NC(=O)[C@H](CC(C)C)n2cc(CCN3CCC3)c(C(F)(F)F)nc2=O)c1F] |
| | 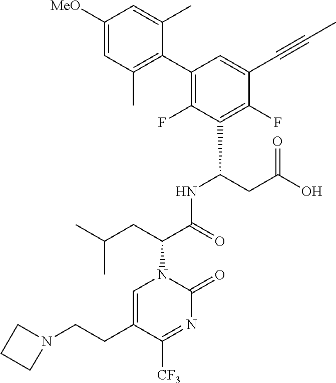 [SMILES: CC#Cc1cc(-c2c(C)cc(OC)cc2C)c(F)c([C@H](CC(=O)O)NC(=O)[C@@H](CC(C)C)n2cc(CCN3CCC3)c(C(F)(F)F)nc2=O)c1F] |
| | 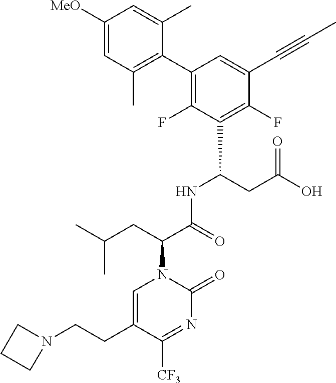 [SMILES: CC#Cc1cc(-c2c(C)cc(OC)cc2C)c(F)c([C@H](CC(=O)O)NC(=O)[C@H](CC(C)C)n2cc(CCN3CCC3)c(C(F)(F)F)nc2=O)c1F] |
| | 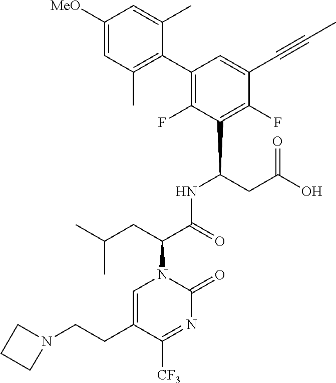 [SMILES: CC#Cc1cc(-c2c(C)cc(OC)cc2C)c(F)c([C@@H](CC(=O)O)NC(=O)[C@H](CC(C)C)n2cc(CCN3CCC3)c(C(F)(F)F)nc2=O)c1F] |
| | 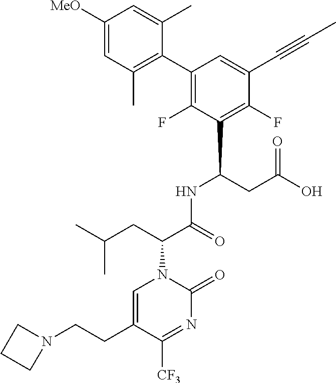 [SMILES: CC#Cc1cc(-c2c(C)cc(OC)cc2C)c(F)c([C@@H](CC(=O)O)NC(=O)[C@@H](CC(C)C)n2cc(CCN3CCC3)c(C(F)(F)F)nc2=O)c1F] |

TABLE E-5

| | | |
|---|---|---|
| 362 | 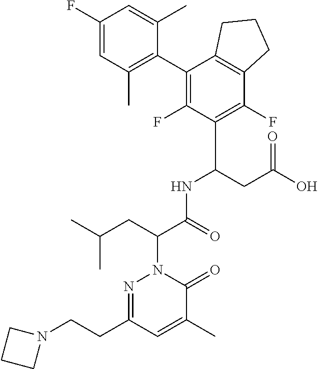 [SMILES: Cc1cc(F)cc(C)c1-c1c(F)c(C(CC(=O)O)NC(=O)C(CC(C)C)n2nc(CCN3CCC3)cc(C)c2=O)c(F)c2c1CCC2] | 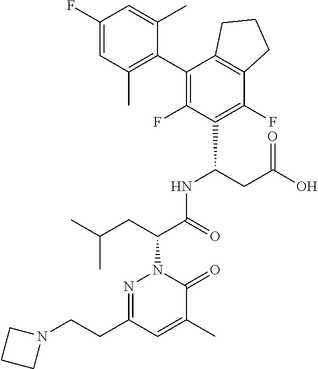 [SMILES: Cc1cc(F)cc(C)c1-c1c(F)c([C@H](CC(=O)O)NC(=O)[C@@H](CC(C)C)n2nc(CCN3CCC3)cc(C)c2=O)c(F)c2c1CCC2] |
| 363 | 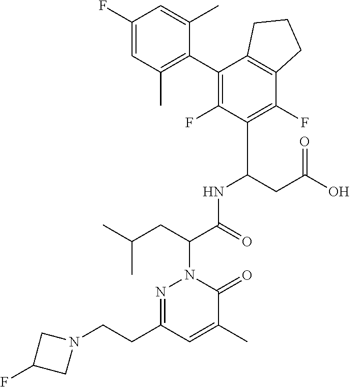 [SMILES: Cc1cc(F)cc(C)c1-c1c(F)c(C(CC(=O)O)NC(=O)C(CC(C)C)n2nc(CCN3CC(F)C3)cc(C)c2=O)c(F)c2c1CCC2] | 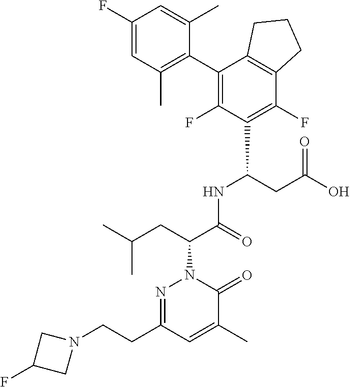 [SMILES: Cc1cc(F)cc(C)c1-c1c(F)c([C@H](CC(=O)O)NC(=O)[C@@H](CC(C)C)n2nc(CCN3CC(F)C3)cc(C)c2=O)c(F)c2c1CCC2] |
| 364 | 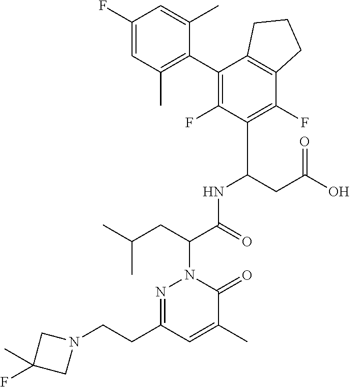 [SMILES: Cc1cc(F)cc(C)c1-c1c(F)c(C(CC(=O)O)NC(=O)C(CC(C)C)n2nc(CCN3CC(C)(F)C3)cc(C)c2=O)c(F)c2c1CCC2] | 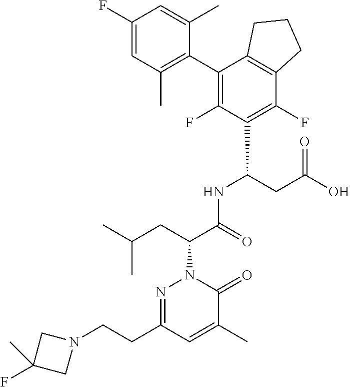 [SMILES: Cc1cc(F)cc(C)c1-c1c(F)c([C@H](CC(=O)O)NC(=O)[C@@H](CC(C)C)n2nc(CCN3CC(C)(F)C3)cc(C)c2=O)c(F)c2c1CCC2] |
| 365 | 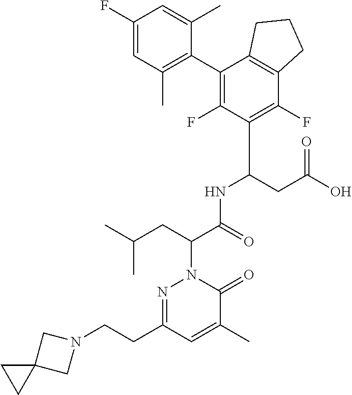 [SMILES: Cc1cc(F)cc(C)c1-c1c(F)c(C(CC(=O)O)NC(=O)C(CC(C)C)n2nc(CCN3CC4(CC4)C3)cc(C)c2=O)c(F)c2c1CCC2] | 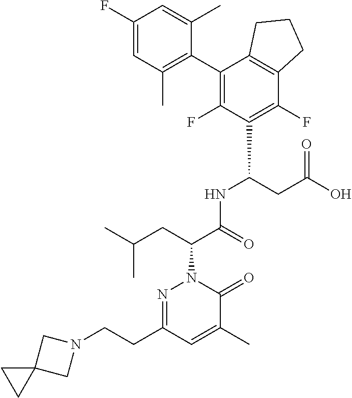 [SMILES: Cc1cc(F)cc(C)c1-c1c(F)c([C@H](CC(=O)O)NC(=O)[C@@H](CC(C)C)n2nc(CCN3CC4(CC4)C3)cc(C)c2=O)c(F)c2c1CCC2] |
| 366 | 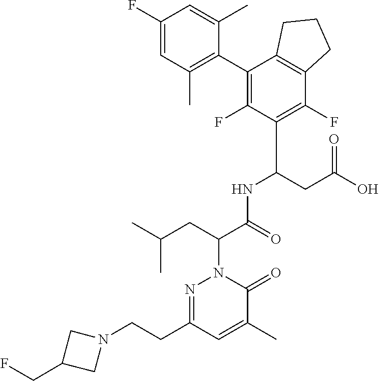 [SMILES: Cc1cc(F)cc(C)c1-c1c(F)c(C(CC(=O)O)NC(=O)C(CC(C)C)n2nc(CCN3CC(CF)C3)cc(C)c2=O)c(F)c2c1CCC2] | 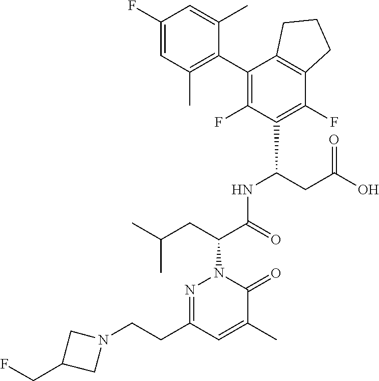 [SMILES: Cc1cc(F)cc(C)c1-c1c(F)c([C@H](CC(=O)O)NC(=O)[C@@H](CC(C)C)n2nc(CCN3CC(CF)C3)cc(C)c2=O)c(F)c2c1CCC2] |
| 367 | 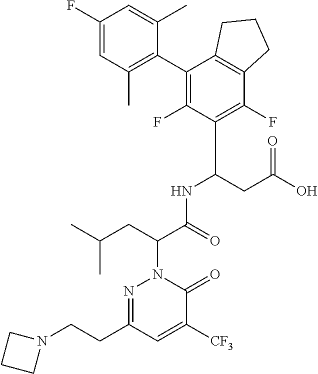 [SMILES: Cc1cc(F)cc(C)c1-c1c(F)c(C(CC(=O)O)NC(=O)C(CC(C)C)n2nc(CCN3CCC3)cc(C(F)(F)F)c2=O)c(F)c2c1CCC2] | 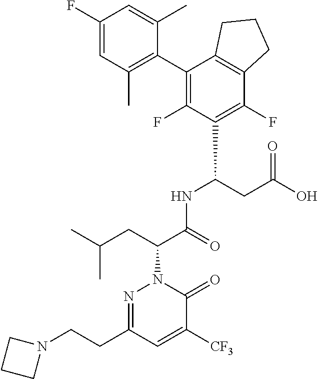 [SMILES: Cc1cc(F)cc(C)c1-c1c(F)c([C@H](CC(=O)O)NC(=O)[C@@H](CC(C)C)n2nc(CCN3CCC3)cc(C(F)(F)F)c2=O)c(F)c2c1CCC2] |
| 368 | 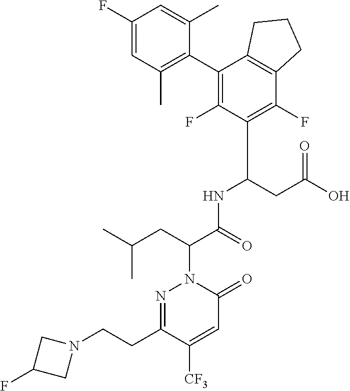 [SMILES: Cc1cc(F)cc(C)c1-c1c(F)c(C(CC(=O)O)NC(=O)C(CC(C)C)n2nc(CCN3CC(F)C3)c(C(F)(F)F)cc2=O)c(F)c2c1CCC2] | 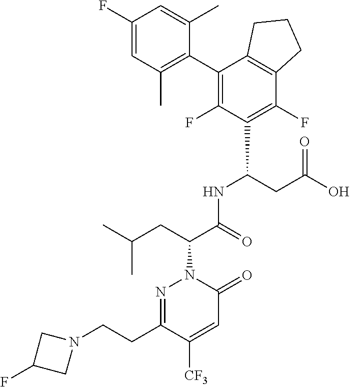 [SMILES: Cc1cc(F)cc(C)c1-c1c(F)c([C@H](CC(=O)O)NC(=O)[C@@H](CC(C)C)n2nc(CCN3CC(F)C3)c(C(F)(F)F)cc2=O)c(F)c2c1CCC2] |
| 369 | 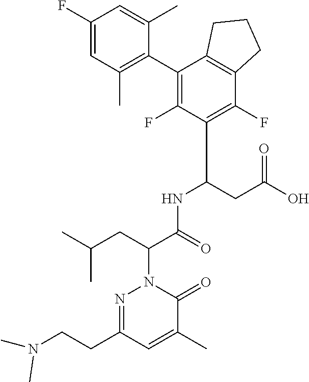 [SMILES: Cc1cc(F)cc(C)c1-c1c(F)c(C(CC(=O)O)NC(=O)C(CC(C)C)n2nc(CCN(C)C)cc(C)c2=O)c(F)c2c1CCC2] | 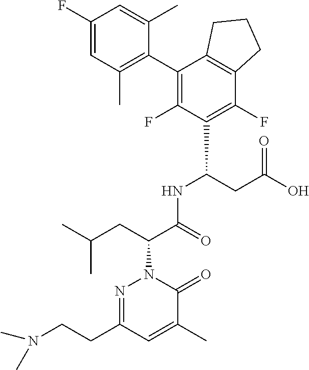 [SMILES: Cc1cc(F)cc(C)c1-c1c(F)c([C@H](CC(=O)O)NC(=O)[C@@H](CC(C)C)n2nc(CCN(C)C)cc(C)c2=O)c(F)c2c1CCC2] |
| 370 | 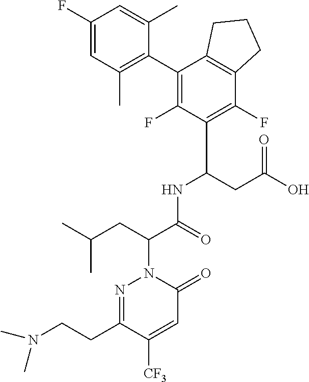 [SMILES: Cc1cc(F)cc(C)c1-c1c(F)c(C(CC(=O)O)NC(=O)C(CC(C)C)n2nc(CCN(C)C)c(C(F)(F)F)cc2=O)c(F)c2c1CCC2] | 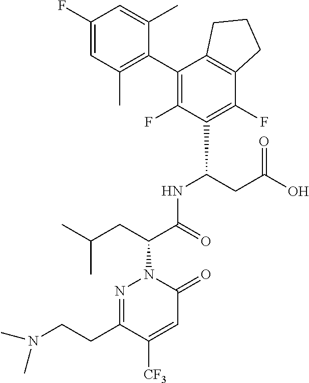 [SMILES: Cc1cc(F)cc(C)c1-c1c(F)c([C@H](CC(=O)O)NC(=O)[C@@H](CC(C)C)n2nc(CCN(C)C)c(C(F)(F)F)cc2=O)c(F)c2c1CCC2] |
| 371 | 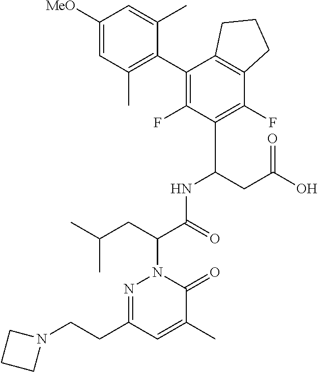 [SMILES: COc1cc(C)c(-c2c(F)c(C(CC(=O)O)NC(=O)C(CC(C)C)n3nc(CCN4CCC4)cc(C)c3=O)c(F)c3c2CCC3)c(C)c1] | 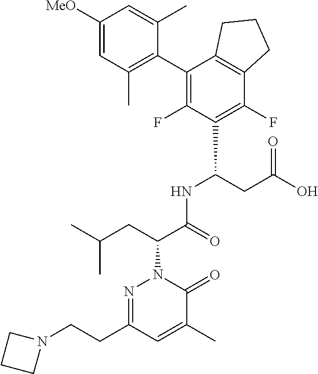 [SMILES: COc1cc(C)c(-c2c(F)c([C@H](CC(=O)O)NC(=O)[C@@H](CC(C)C)n3nc(CCN4CCC4)cc(C)c3=O)c(F)c3c2CCC3)c(C)c1] |
| 372 | 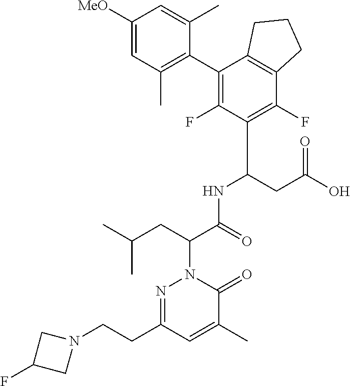 [SMILES: COc1cc(C)c(-c2c(F)c(C(CC(=O)O)NC(=O)C(CC(C)C)n3nc(CCN4CC(F)C4)cc(C)c3=O)c(F)c3c2CCC3)c(C)c1] | 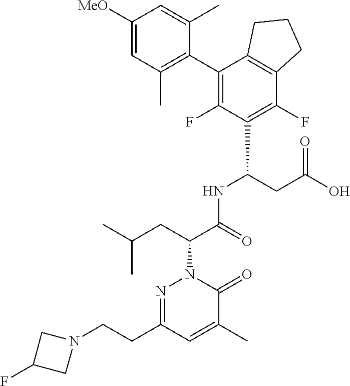 [SMILES: COc1cc(C)c(-c2c(F)c([C@H](CC(=O)O)NC(=O)[C@@H](CC(C)C)n3nc(CCN4CC(F)C4)cc(C)c3=O)c(F)c3c2CCC3)c(C)c1] |
| 373 | 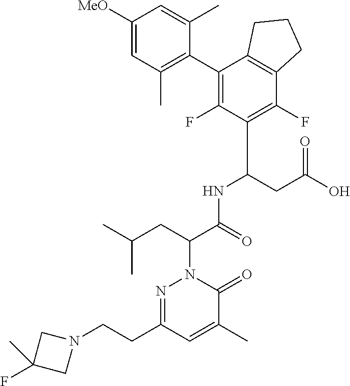 [SMILES: COc1cc(C)c(-c2c(F)c(C(CC(=O)O)NC(=O)C(CC(C)C)n3nc(CCN4CC(C)(F)C4)cc(C)c3=O)c(F)c3c2CCC3)c(C)c1] | 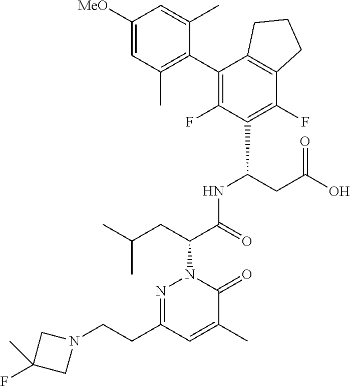 [SMILES: COc1cc(C)c(-c2c(F)c([C@H](CC(=O)O)NC(=O)[C@@H](CC(C)C)n3nc(CCN4CC(C)(F)C4)cc(C)c3=O)c(F)c3c2CCC3)c(C)c1] |
| 374 | 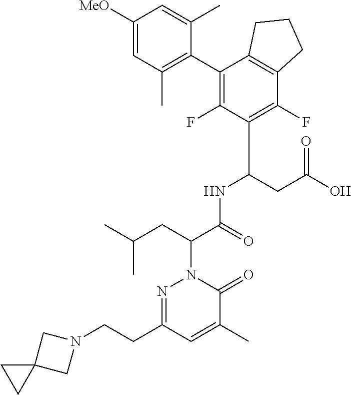 [SMILES: COc1cc(C)c(-c2c(F)c(C(CC(=O)O)NC(=O)C(CC(C)C)n3nc(CCN4CC5(CC5)C4)cc(C)c3=O)c(F)c3c2CCC3)c(C)c1] | 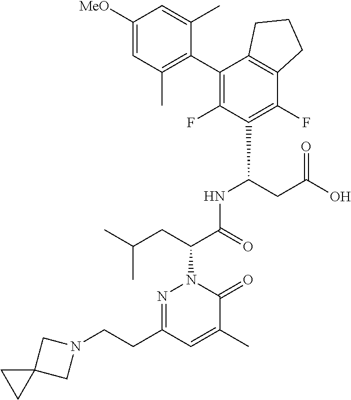 [SMILES: COc1cc(C)c(-c2c(F)c([C@H](CC(=O)O)NC(=O)[C@@H](CC(C)C)n3nc(CCN4CC5(CC5)C4)cc(C)c3=O)c(F)c3c2CCC3)c(C)c1] |
| 375 | 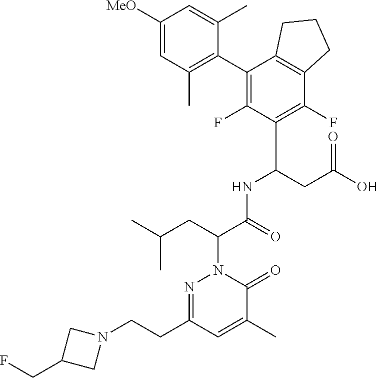 [SMILES: COc1cc(C)c(-c2c(F)c(C(CC(=O)O)NC(=O)C(CC(C)C)n3nc(CCN4CC(CF)C4)cc(C)c3=O)c(F)c3c2CCC3)c(C)c1] | 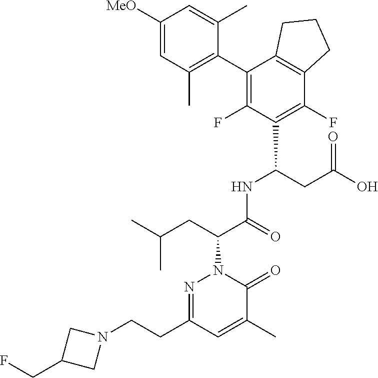 [SMILES: COc1cc(C)c(-c2c(F)c([C@H](CC(=O)O)NC(=O)[C@@H](CC(C)C)n3nc(CCN4CC(CF)C4)cc(C)c3=O)c(F)c3c2CCC3)c(C)c1] |
| 376 | 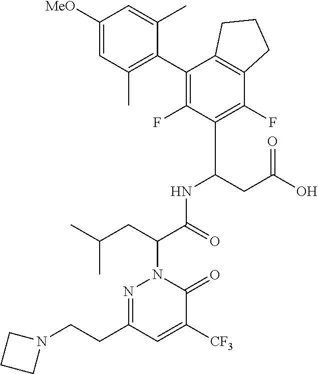 [SMILES: COc1cc(C)c(-c2c(F)c(C(CC(=O)O)NC(=O)C(CC(C)C)n3nc(CCN4CCC4)cc(C(F)(F)F)c3=O)c(F)c3c2CCC3)c(C)c1] | 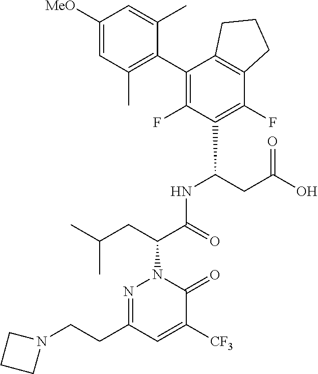 [SMILES: COc1cc(C)c(-c2c(F)c([C@H](CC(=O)O)NC(=O)[C@@H](CC(C)C)n3nc(CCN4CCC4)cc(C(F)(F)F)c3=O)c(F)c3c2CCC3)c(C)c1] |
| 377 | 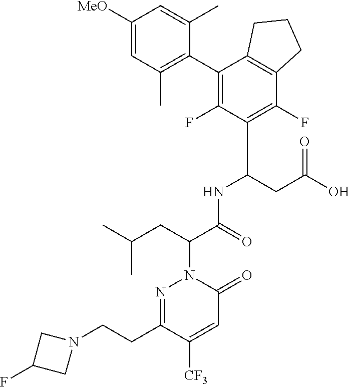 [SMILES: COc1cc(C)c(-c2c(F)c(C(CC(=O)O)NC(=O)C(CC(C)C)n3nc(CCN4CC(F)C4)c(C(F)(F)F)cc3=O)c(F)c3c2CCC3)c(C)c1] | 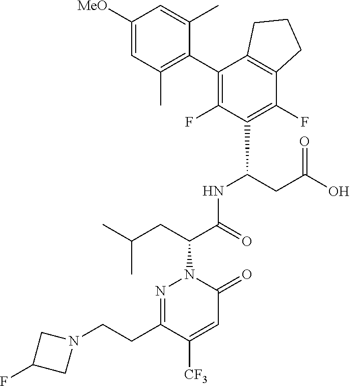 [SMILES: COc1cc(C)c(-c2c(F)c([C@H](CC(=O)O)NC(=O)[C@@H](CC(C)C)n3nc(CCN4CC(F)C4)c(C(F)(F)F)cc3=O)c(F)c3c2CCC3)c(C)c1] |
| 378 | 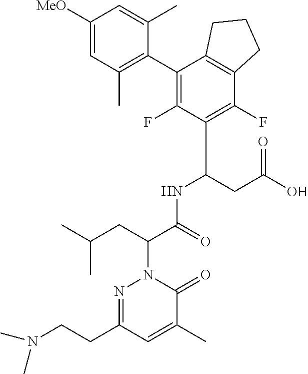 [SMILES: COc1cc(C)c(-c2c(F)c(C(CC(=O)O)NC(=O)C(CC(C)C)n3nc(CCN(C)C)cc(C)c3=O)c(F)c3c2CCC3)c(C)c1] | 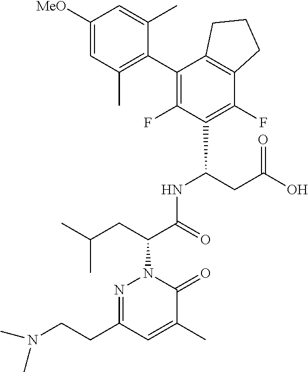 [SMILES: COc1cc(C)c(-c2c(F)c([C@H](CC(=O)O)NC(=O)[C@@H](CC(C)C)n3nc(CCN(C)C)cc(C)c3=O)c(F)c3c2CCC3)c(C)c1] |
| 379 | 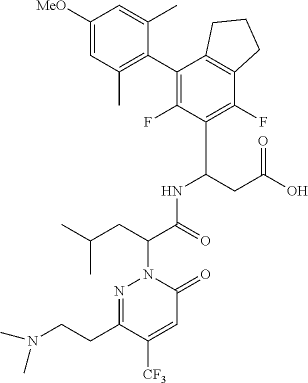 [SMILES: COc1cc(C)c(-c2c(F)c(C(CC(=O)O)NC(=O)C(CC(C)C)n3nc(CCN(C)C)c(C(F)(F)F)cc3=O)c(F)c3c2CCC3)c(C)c1] | 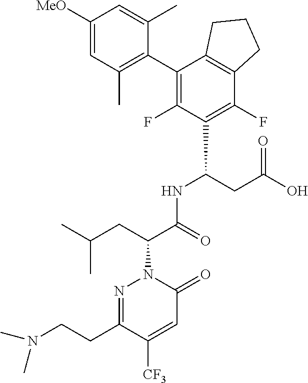 [SMILES: COc1cc(C)c(-c2c(F)c([C@H](CC(=O)O)NC(=O)[C@@H](CC(C)C)n3nc(CCN(C)C)c(C(F)(F)F)cc3=O)c(F)c3c2CCC3)c(C)c1] |
| 380 | 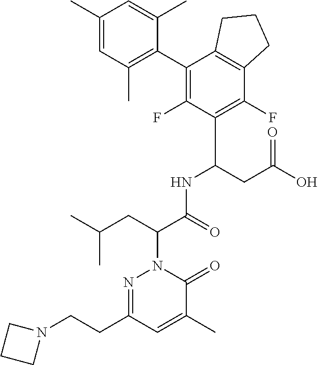 [SMILES: Cc1cc(C)c(-c2c(F)c(C(CC(=O)O)NC(=O)C(CC(C)C)n3nc(CCN4CCC4)cc(C)c3=O)c(F)c3c2CCC3)c(C)c1] | 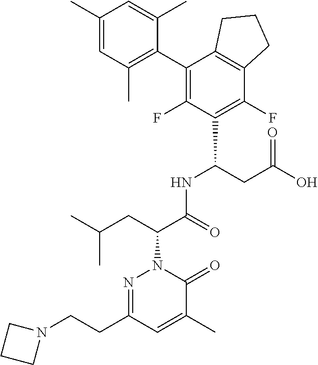 [SMILES: Cc1cc(C)c(-c2c(F)c([C@H](CC(=O)O)NC(=O)[C@@H](CC(C)C)n3nc(CCN4CCC4)cc(C)c3=O)c(F)c3c2CCC3)c(C)c1] |
| 381 | 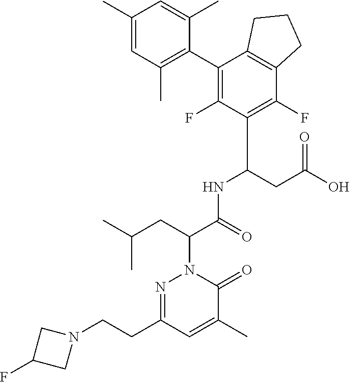 [SMILES: Cc1cc(C)c(-c2c(F)c(C(CC(=O)O)NC(=O)C(CC(C)C)n3nc(CCN4CC(F)C4)cc(C)c3=O)c(F)c3c2CCC3)c(C)c1] | 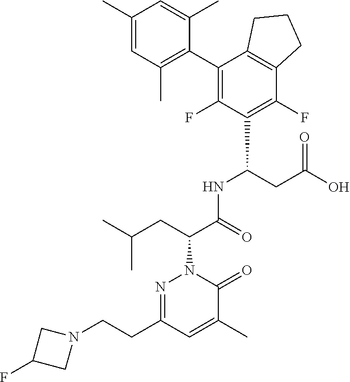 [SMILES: Cc1cc(C)c(-c2c(F)c([C@H](CC(=O)O)NC(=O)[C@@H](CC(C)C)n3nc(CCN4CC(F)C4)cc(C)c3=O)c(F)c3c2CCC3)c(C)c1] |
| 382 | 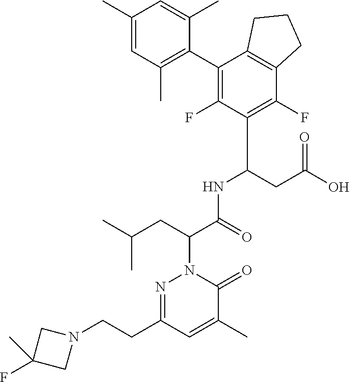 [SMILES: Cc1cc(C)c(-c2c(F)c(C(CC(=O)O)NC(=O)C(CC(C)C)n3nc(CCN4CC(C)(F)C4)cc(C)c3=O)c(F)c3c2CCC3)c(C)c1] | 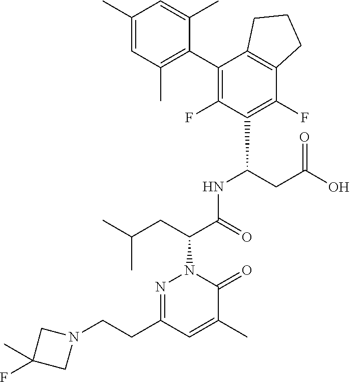 [SMILES: Cc1cc(C)c(-c2c(F)c([C@H](CC(=O)O)NC(=O)[C@@H](CC(C)C)n3nc(CCN4CC(C)(F)C4)cc(C)c3=O)c(F)c3c2CCC3)c(C)c1] |
| 383 | 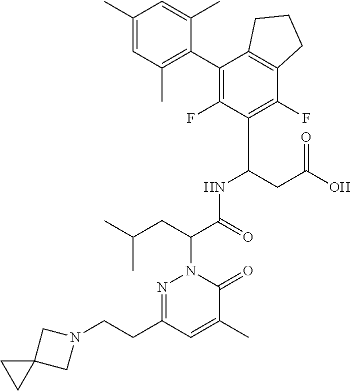 [SMILES: Cc1cc(C)c(-c2c(F)c(C(CC(=O)O)NC(=O)C(CC(C)C)n3nc(CCN4CC5(CC5)C4)cc(C)c3=O)c(F)c3c2CCC3)c(C)c1] | 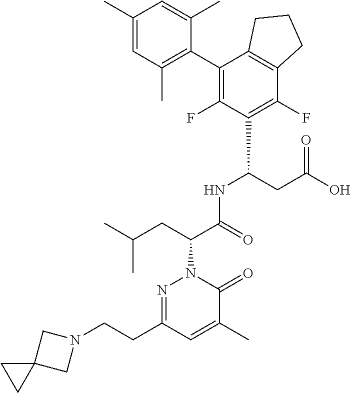 [SMILES: Cc1cc(C)c(-c2c(F)c([C@H](CC(=O)O)NC(=O)[C@@H](CC(C)C)n3nc(CCN4CC5(CC5)C4)cc(C)c3=O)c(F)c3c2CCC3)c(C)c1] |
| 384 | 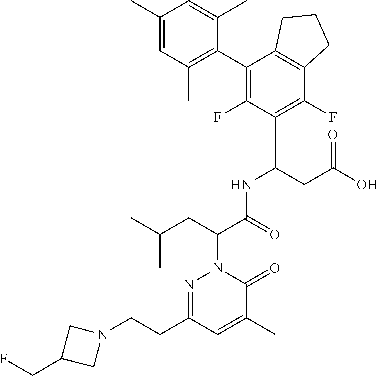 [SMILES: Cc1cc(C)c(-c2c(F)c(C(CC(=O)O)NC(=O)C(CC(C)C)n3nc(CCN4CC(CF)C4)cc(C)c3=O)c(F)c3c2CCC3)c(C)c1] | 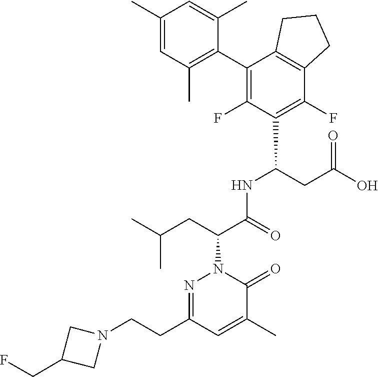 [SMILES: Cc1cc(C)c(-c2c(F)c([C@H](CC(=O)O)NC(=O)[C@@H](CC(C)C)n3nc(CCN4CC(CF)C4)cc(C)c3=O)c(F)c3c2CCC3)c(C)c1] |
| 385 | 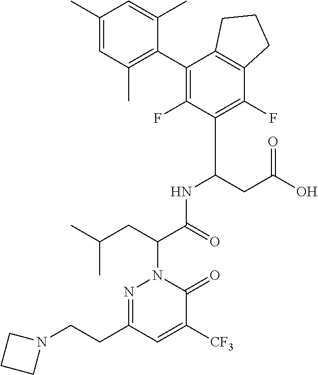 [SMILES: Cc1cc(C)c(-c2c(F)c(C(CC(=O)O)NC(=O)C(CC(C)C)n3nc(CCN4CCC4)cc(C(F)(F)F)c3=O)c(F)c3c2CCC3)c(C)c1] | 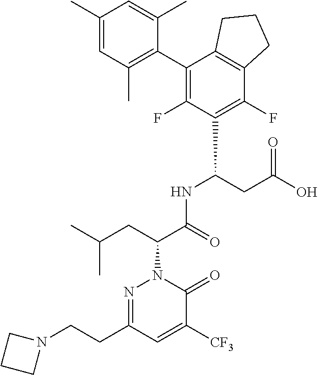 [SMILES: Cc1cc(C)c(-c2c(F)c([C@H](CC(=O)O)NC(=O)[C@@H](CC(C)C)n3nc(CCN4CCC4)cc(C(F)(F)F)c3=O)c(F)c3c2CCC3)c(C)c1] |
| 386 | 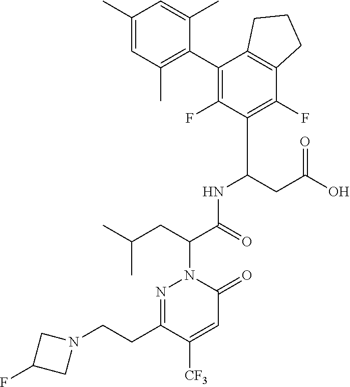 [SMILES: Cc1cc(C)c(-c2c(F)c(C(CC(=O)O)NC(=O)C(CC(C)C)n3nc(CCN4CC(F)C4)c(C(F)(F)F)cc3=O)c(F)c3c2CCC3)c(C)c1] | 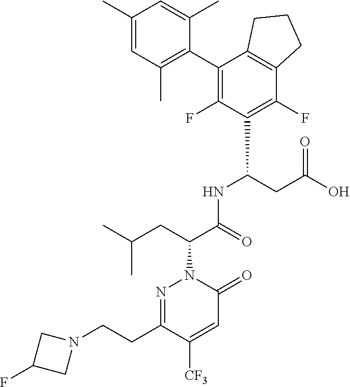 [SMILES: Cc1cc(C)c(-c2c(F)c([C@H](CC(=O)O)NC(=O)[C@@H](CC(C)C)n3nc(CCN4CC(F)C4)c(C(F)(F)F)cc3=O)c(F)c3c2CCC3)c(C)c1] |
| 387 | 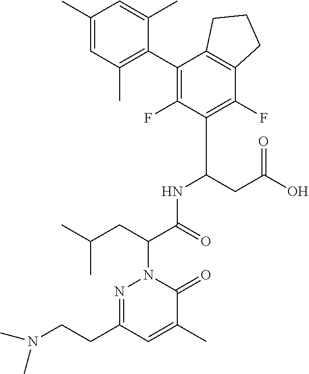 [SMILES: Cc1cc(C)c(-c2c(F)c(C(CC(=O)O)NC(=O)C(CC(C)C)n3nc(CCN(C)C)cc(C)c3=O)c(F)c3c2CCC3)c(C)c1] | 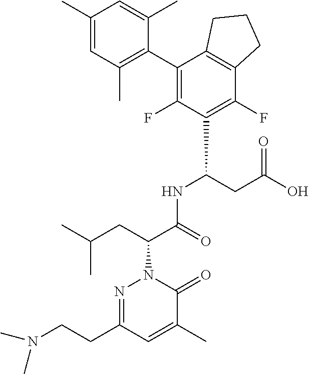 [SMILES: Cc1cc(C)c(-c2c(F)c([C@H](CC(=O)O)NC(=O)[C@@H](CC(C)C)n3nc(CCN(C)C)cc(C)c3=O)c(F)c3c2CCC3)c(C)c1] |
| 388 | 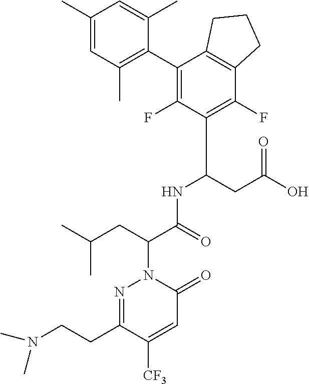 [SMILES: Cc1cc(C)c(-c2c(F)c(C(CC(=O)O)NC(=O)C(CC(C)C)n3nc(CCN(C)C)c(C(F)(F)F)cc3=O)c(F)c3c2CCC3)c(C)c1] | 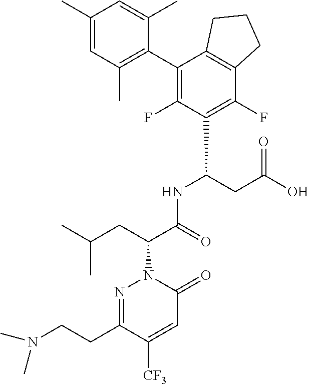 [SMILES: Cc1cc(C)c(-c2c(F)c([C@H](CC(=O)O)NC(=O)[C@@H](CC(C)C)n3nc(CCN(C)C)c(C(F)(F)F)cc3=O)c(F)c3c2CCC3)c(C)c1] |
| 389 | 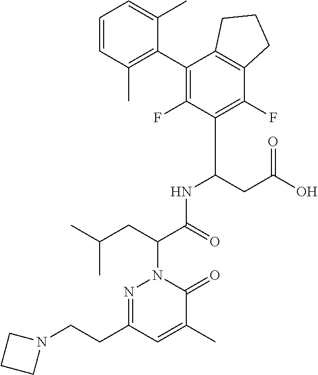 [SMILES: Cc1cccc(C)c1-c1c(F)c(C(CC(=O)O)NC(=O)C(CC(C)C)n2nc(CCN3CCC3)cc(C)c2=O)c(F)c2c1CCC2] | 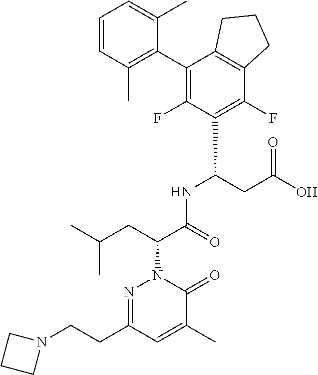 [SMILES: Cc1cccc(C)c1-c1c(F)c([C@H](CC(=O)O)NC(=O)[C@@H](CC(C)C)n2nc(CCN3CCC3)cc(C)c2=O)c(F)c2c1CCC2] |
| 390 | 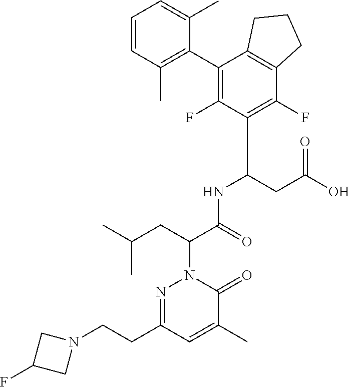 [SMILES: Cc1cccc(C)c1-c1c(F)c(C(CC(=O)O)NC(=O)C(CC(C)C)n2nc(CCN3CC(F)C3)cc(C)c2=O)c(F)c2c1CCC2] | 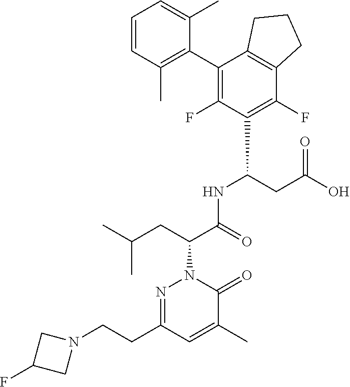 [SMILES: Cc1cccc(C)c1-c1c(F)c([C@H](CC(=O)O)NC(=O)[C@@H](CC(C)C)n2nc(CCN3CC(F)C3)cc(C)c2=O)c(F)c2c1CCC2] |
| 391 | 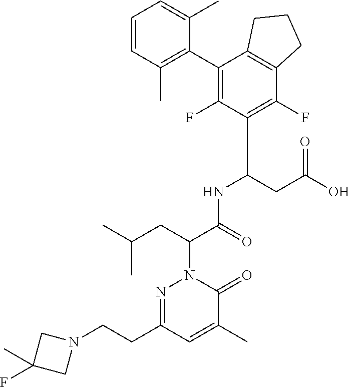 [SMILES: Cc1cccc(C)c1-c1c(F)c(C(CC(=O)O)NC(=O)C(CC(C)C)n2nc(CCN3CC(C)(F)C3)cc(C)c2=O)c(F)c2c1CCC2] | 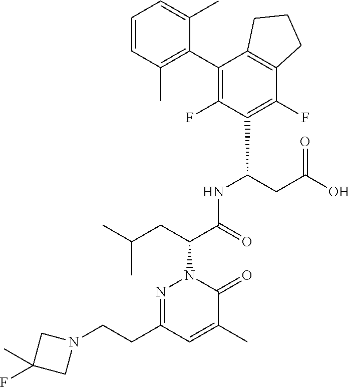 [SMILES: Cc1cccc(C)c1-c1c(F)c([C@H](CC(=O)O)NC(=O)[C@@H](CC(C)C)n2nc(CCN3CC(C)(F)C3)cc(C)c2=O)c(F)c2c1CCC2] |
| 392 | 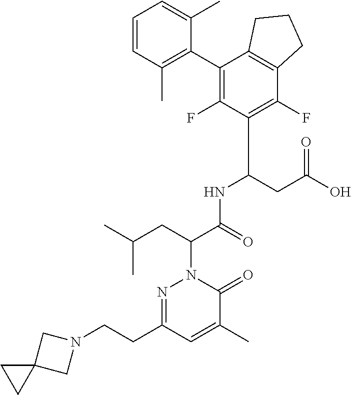 [SMILES: Cc1cccc(C)c1-c1c(F)c(C(CC(=O)O)NC(=O)C(CC(C)C)n2nc(CCN3CC4(CC4)C3)cc(C)c2=O)c(F)c2c1CCC2] | 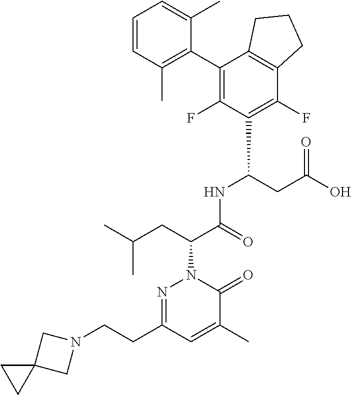 [SMILES: Cc1cccc(C)c1-c1c(F)c([C@H](CC(=O)O)NC(=O)[C@@H](CC(C)C)n2nc(CCN3CC4(CC4)C3)cc(C)c2=O)c(F)c2c1CCC2] |
| 393 | 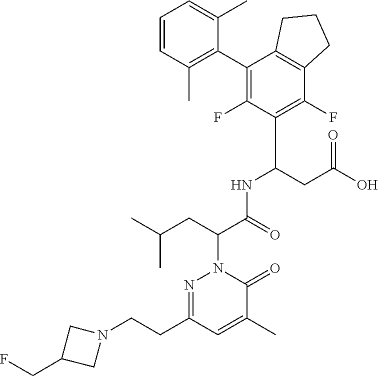 [SMILES: Cc1cccc(C)c1-c1c(F)c(C(CC(=O)O)NC(=O)C(CC(C)C)n2nc(CCN3CC(CF)C3)cc(C)c2=O)c(F)c2c1CCC2] | 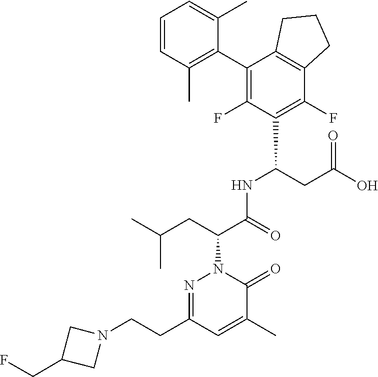 [SMILES: Cc1cccc(C)c1-c1c(F)c([C@H](CC(=O)O)NC(=O)[C@@H](CC(C)C)n2nc(CCN3CC(CF)C3)cc(C)c2=O)c(F)c2c1CCC2] |
| 394 | 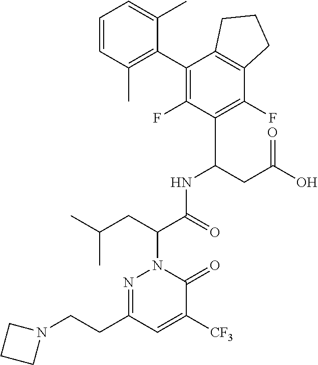 [SMILES: Cc1cccc(C)c1-c1c(F)c(C(CC(=O)O)NC(=O)C(CC(C)C)n2nc(CCN3CCC3)cc(C(F)(F)F)c2=O)c(F)c2c1CCC2] | 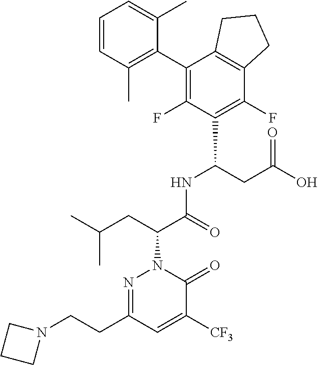 [SMILES: Cc1cccc(C)c1-c1c(F)c([C@H](CC(=O)O)NC(=O)[C@@H](CC(C)C)n2nc(CCN3CCC3)cc(C(F)(F)F)c2=O)c(F)c2c1CCC2] |
| 395 | 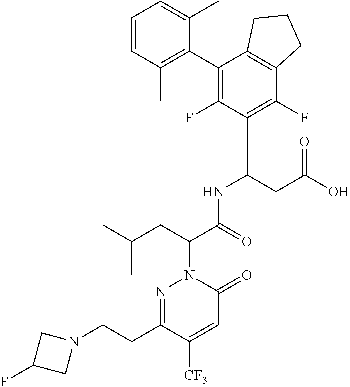 [SMILES: Cc1cccc(C)c1-c1c(F)c(C(CC(=O)O)NC(=O)C(CC(C)C)n2nc(CCN3CC(F)C3)c(C(F)(F)F)cc2=O)c(F)c2c1CCC2] | 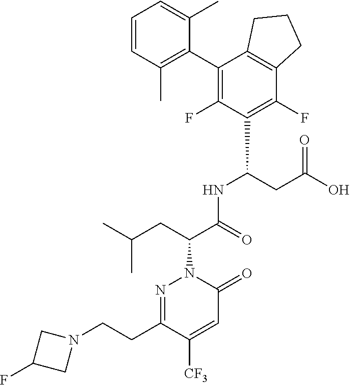 [SMILES: Cc1cccc(C)c1-c1c(F)c([C@H](CC(=O)O)NC(=O)[C@@H](CC(C)C)n2nc(CCN3CC(F)C3)c(C(F)(F)F)cc2=O)c(F)c2c1CCC2] |
| 396 | 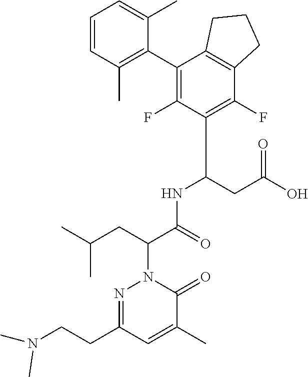 [SMILES: Cc1cccc(C)c1-c1c(F)c(C(CC(=O)O)NC(=O)C(CC(C)C)n2nc(CCN(C)C)cc(C)c2=O)c(F)c2c1CCC2] | 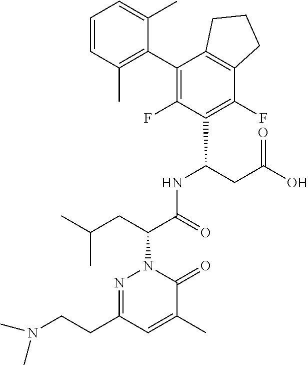 [SMILES: Cc1cccc(C)c1-c1c(F)c([C@H](CC(=O)O)NC(=O)[C@@H](CC(C)C)n2nc(CCN(C)C)cc(C)c2=O)c(F)c2c1CCC2] |
| 397 | 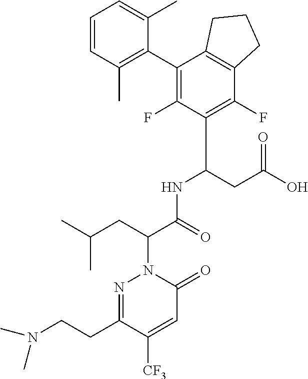 [SMILES: Cc1cccc(C)c1-c1c(F)c(C(CC(=O)O)NC(=O)C(CC(C)C)n2nc(CCN(C)C)c(C(F)(F)F)cc2=O)c(F)c2c1CCC2] | 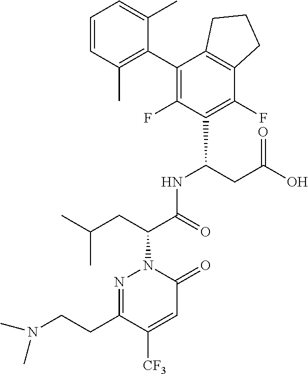 [SMILES: Cc1cccc(C)c1-c1c(F)c([C@H](CC(=O)O)NC(=O)[C@@H](CC(C)C)n2nc(CCN(C)C)c(C(F)(F)F)cc2=O)c(F)c2c1CCC2] |
| 398 | 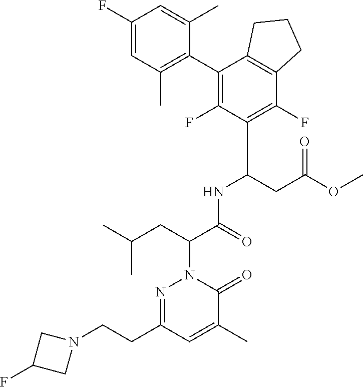 [SMILES: COC(=O)CC(NC(=O)C(CC(C)C)n1nc(CCN2CC(F)C2)cc(C)c1=O)c1c(F)c2c(c(-c3c(C)cc(F)cc3C)c1F)CCC2] | 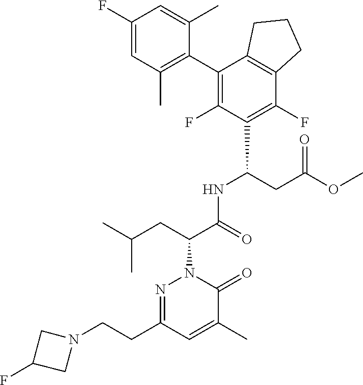 [SMILES: COC(=O)C[C@H](NC(=O)[C@@H](CC(C)C)n1nc(CCN2CC(F)C2)cc(C)c1=O)c1c(F)c2c(c(-c3c(C)cc(F)cc3C)c1F)CCC2] |
| 399 | 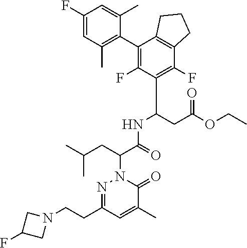 [SMILES: CCOC(=O)CC(NC(=O)C(CC(C)C)n1nc(CCN2CC(F)C2)cc(C)c1=O)c1c(F)c2c(c(-c3c(C)cc(F)cc3C)c1F)CCC2] | 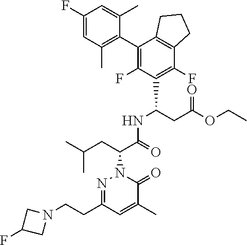 [SMILES: CCOC(=O)C[C@H](NC(=O)[C@@H](CC(C)C)n1nc(CCN2CC(F)C2)cc(C)c1=O)c1c(F)c2c(c(-c3c(C)cc(F)cc3C)c1F)CCC2] |
| 400 | 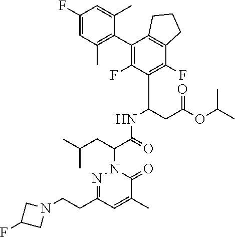 [SMILES: Cc1cc(F)cc(C)c1-c1c(F)c(C(CC(=O)OC(C)C)NC(=O)C(CC(C)C)n2nc(CCN3CC(F)C3)cc(C)c2=O)c(F)c2c1CCC2] | 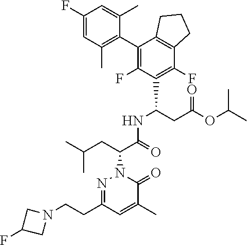 [SMILES: Cc1cc(F)cc(C)c1-c1c(F)c([C@H](CC(=O)OC(C)C)NC(=O)[C@@H](CC(C)C)n2nc(CCN3CC(F)C3)cc(C)c2=O)c(F)c2c1CCC2] |
| 401 | 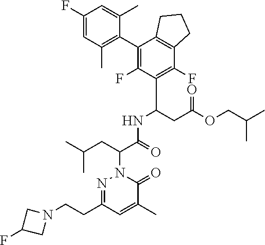 [SMILES: Cc1cc(F)cc(C)c1-c1c(F)c(C(CC(=O)OCC(C)C)NC(=O)C(CC(C)C)n2nc(CCN3CC(F)C3)cc(C)c2=O)c(F)c2c1CCC2] | 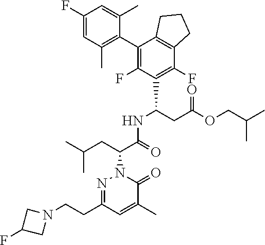 [SMILES: Cc1cc(F)cc(C)c1-c1c(F)c([C@H](CC(=O)OCC(C)C)NC(=O)[C@@H](CC(C)C)n2nc(CCN3CC(F)C3)cc(C)c2=O)c(F)c2c1CCC2] |
| 402 | 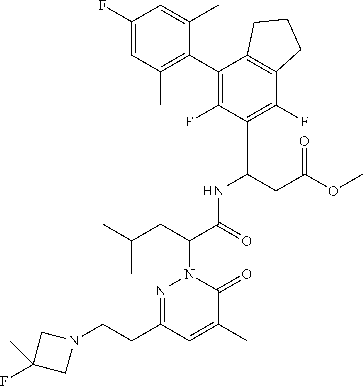 [SMILES: COC(=O)CC(NC(=O)C(CC(C)C)n1nc(CCN2CC(C)(F)C2)cc(C)c1=O)c1c(F)c2c(c(-c3c(C)cc(F)cc3C)c1F)CCC2] | 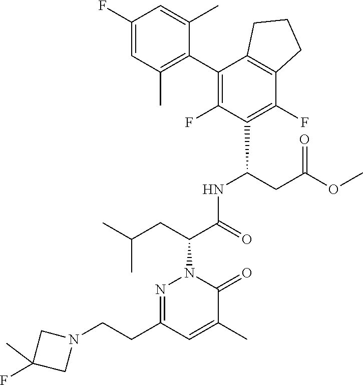 [SMILES: COC(=O)C[C@H](NC(=O)[C@@H](CC(C)C)n1nc(CCN2CC(C)(F)C2)cc(C)c1=O)c1c(F)c2c(c(-c3c(C)cc(F)cc3C)c1F)CCC2] |
| 403 | 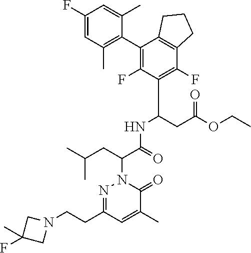 [SMILES: CCOC(=O)CC(NC(=O)C(CC(C)C)n1nc(CCN2CC(C)(F)C2)cc(C)c1=O)c1c(F)c2c(c(-c3c(C)cc(F)cc3C)c1F)CCC2] | 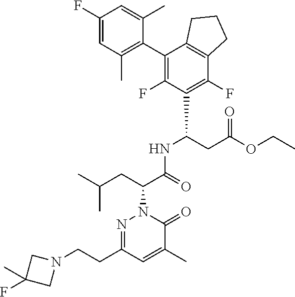 [SMILES: CCOC(=O)C[C@H](NC(=O)[C@@H](CC(C)C)n1nc(CCN2CC(C)(F)C2)cc(C)c1=O)c1c(F)c2c(c(-c3c(C)cc(F)cc3C)c1F)CCC2] |
| 404 | 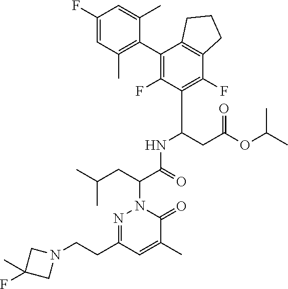 [SMILES: Cc1cc(F)cc(C)c1-c1c(F)c(C(CC(=O)OC(C)C)NC(=O)C(CC(C)C)n2nc(CCN3CC(C)(F)C3)cc(C)c2=O)c(F)c2c1CCC2] | 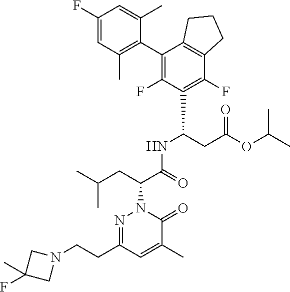 [SMILES: Cc1cc(F)cc(C)c1-c1c(F)c([C@H](CC(=O)OC(C)C)NC(=O)[C@@H](CC(C)C)n2nc(CCN3CC(C)(F)C3)cc(C)c2=O)c(F)c2c1CCC2] |
| 405 | 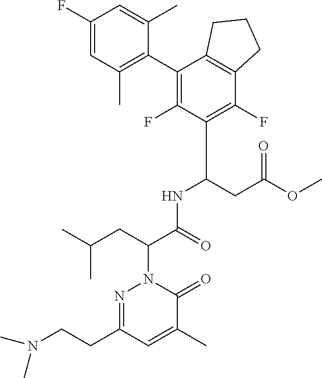 | 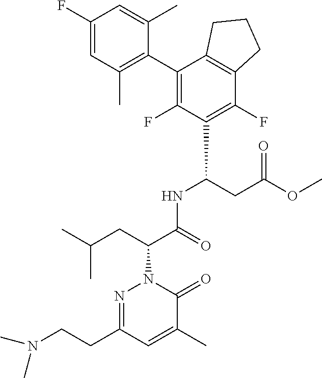 |
| 406 | 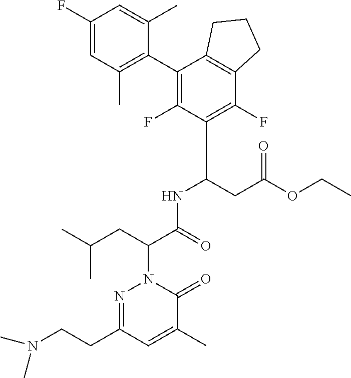 [SMILES: CCOC(=O)CC(NC(=O)C(CC(C)C)n1nc(CCN(C)C)cc(C)c1=O)c1c(F)c2c(c(-c3c(C)cc(F)cc3C)c1F)CCC2] | 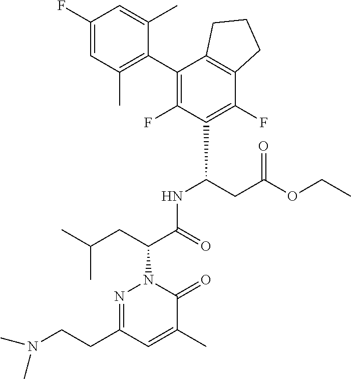 [SMILES: CCOC(=O)C[C@H](NC(=O)[C@@H](CC(C)C)n1nc(CCN(C)C)cc(C)c1=O)c1c(F)c2c(c(-c3c(C)cc(F)cc3C)c1F)CCC2] |
| 407 | 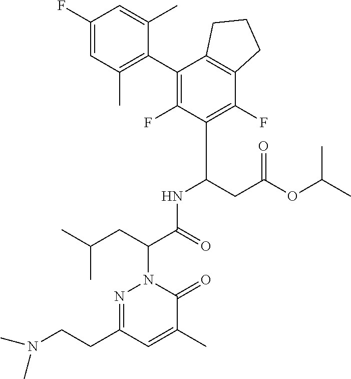 [SMILES: Cc1cc(F)cc(C)c1-c1c(F)c(C(CC(=O)OC(C)C)NC(=O)C(CC(C)C)n2nc(CCN(C)C)cc(C)c2=O)c(F)c2c1CCC2] | 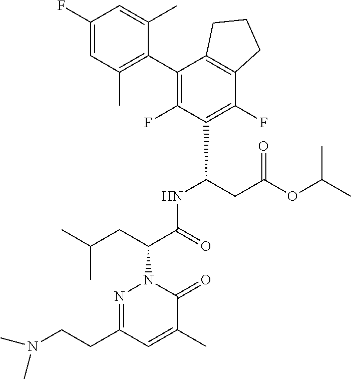 [SMILES: Cc1cc(F)cc(C)c1-c1c(F)c([C@H](CC(=O)OC(C)C)NC(=O)[C@@H](CC(C)C)n2nc(CCN(C)C)cc(C)c2=O)c(F)c2c1CCC2] |
| 408 | 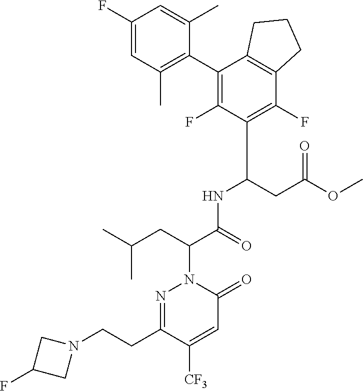 [SMILES: COC(=O)CC(NC(=O)C(CC(C)C)n1nc(CCN2CC(F)C2)c(C(F)(F)F)cc1=O)c1c(F)c2c(c(-c3c(C)cc(F)cc3C)c1F)CCC2] | 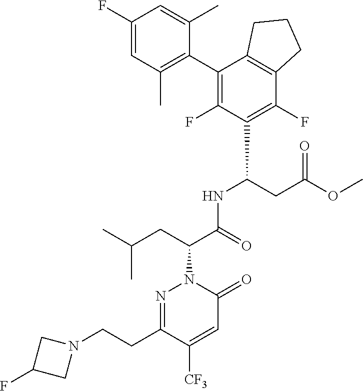 [SMILES: COC(=O)C[C@H](NC(=O)[C@@H](CC(C)C)n1nc(CCN2CC(F)C2)c(C(F)(F)F)cc1=O)c1c(F)c2c(c(-c3c(C)cc(F)cc3C)c1F)CCC2] |
| 409 | 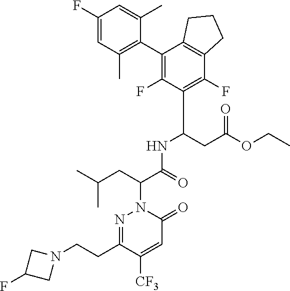 [SMILES: CCOC(=O)CC(NC(=O)C(CC(C)C)n1nc(CCN2CC(F)C2)c(C(F)(F)F)cc1=O)c1c(F)c2c(c(-c3c(C)cc(F)cc3C)c1F)CCC2] | 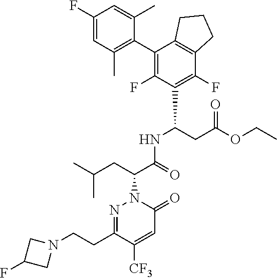 [SMILES: CCOC(=O)C[C@H](NC(=O)[C@@H](CC(C)C)n1nc(CCN2CC(F)C2)c(C(F)(F)F)cc1=O)c1c(F)c2c(c(-c3c(C)cc(F)cc3C)c1F)CCC2] |
| 410 | 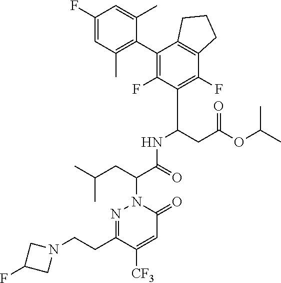 [SMILES: Cc1cc(F)cc(C)c1-c1c(F)c(C(CC(=O)OC(C)C)NC(=O)C(CC(C)C)n2nc(CCN3CC(F)C3)c(C(F)(F)F)cc2=O)c(F)c2c1CCC2] | 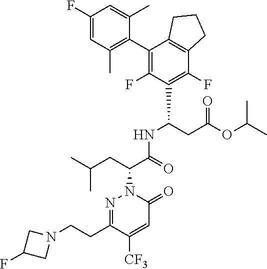 [SMILES: Cc1cc(F)cc(C)c1-c1c(F)c([C@H](CC(=O)OC(C)C)NC(=O)[C@@H](CC(C)C)n2nc(CCN3CC(F)C3)c(C(F)(F)F)cc2=O)c(F)c2c1CCC2] |
| 411 | 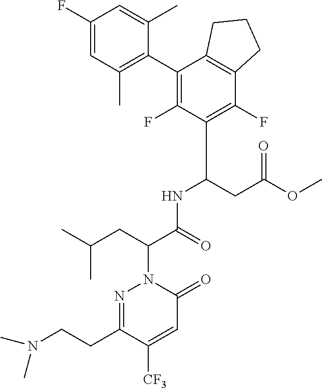 | 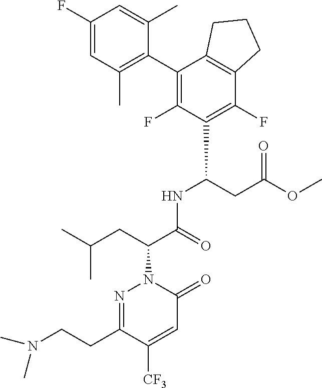 |
| 412 | 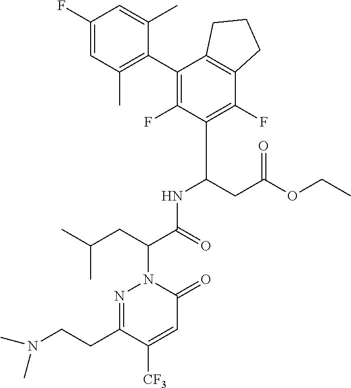 [SMILES: CCOC(=O)CC(NC(=O)C(CC(C)C)n1nc(CCN(C)C)c(C(F)(F)F)cc1=O)c1c(F)c2c(c(-c3c(C)cc(F)cc3C)c1F)CCC2] | 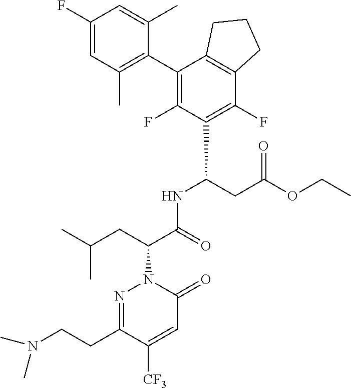 [SMILES: CCOC(=O)C[C@H](NC(=O)[C@@H](CC(C)C)n1nc(CCN(C)C)c(C(F)(F)F)cc1=O)c1c(F)c2c(c(-c3c(C)cc(F)cc3C)c1F)CCC2] |
| 413 | 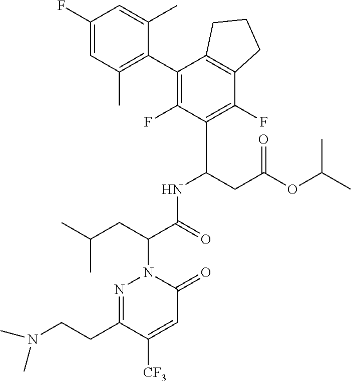 [SMILES: Cc1cc(F)cc(C)c1-c1c(F)c(C(CC(=O)OC(C)C)NC(=O)C(CC(C)C)n2nc(CCN(C)C)c(C(F)(F)F)cc2=O)c(F)c2c1CCC2] | 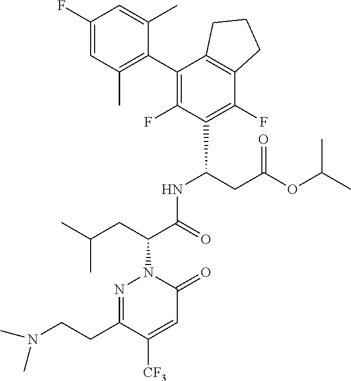 [SMILES: Cc1cc(F)cc(C)c1-c1c(F)c([C@H](CC(=O)OC(C)C)NC(=O)[C@@H](CC(C)C)n2nc(CCN(C)C)c(C(F)(F)F)cc2=O)c(F)c2c1CCC2] |
| 414 | 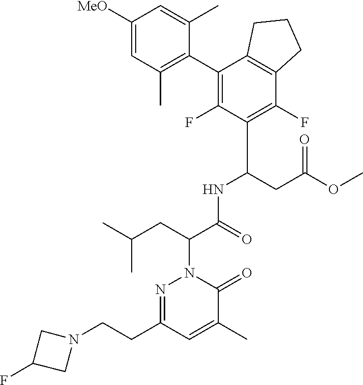 [SMILES: COC(=O)CC(NC(=O)C(CC(C)C)n1nc(CCN2CC(F)C2)cc(C)c1=O)c1c(F)c2c(c(-c3c(C)cc(OC)cc3C)c1F)CCC2] | 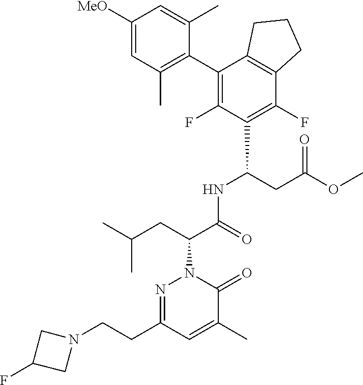 [SMILES: COC(=O)C[C@H](NC(=O)[C@@H](CC(C)C)n1nc(CCN2CC(F)C2)cc(C)c1=O)c1c(F)c2c(c(-c3c(C)cc(OC)cc3C)c1F)CCC2] |
| 415 | 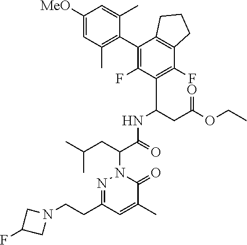 [SMILES: CCOC(=O)CC(NC(=O)C(CC(C)C)n1nc(CCN2CC(F)C2)cc(C)c1=O)c1c(F)c2c(c(-c3c(C)cc(OC)cc3C)c1F)CCC2] | 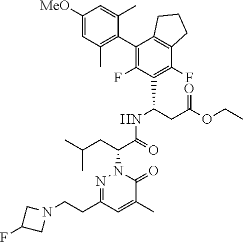 [SMILES: CCOC(=O)C[C@H](NC(=O)[C@@H](CC(C)C)n1nc(CCN2CC(F)C2)cc(C)c1=O)c1c(F)c2c(c(-c3c(C)cc(OC)cc3C)c1F)CCC2] |
| 416 | 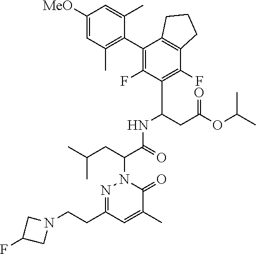 [SMILES: COc1cc(C)c(-c2c(F)c(C(CC(=O)OC(C)C)NC(=O)C(CC(C)C)n3nc(CCN4CC(F)C4)cc(C)c3=O)c(F)c3c2CCC3)c(C)c1] | 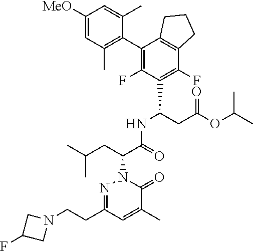 [SMILES: COc1cc(C)c(-c2c(F)c([C@H](CC(=O)OC(C)C)NC(=O)[C@@H](CC(C)C)n3nc(CCN4CC(F)C4)cc(C)c3=O)c(F)c3c2CCC3)c(C)c1] |
| 417 | 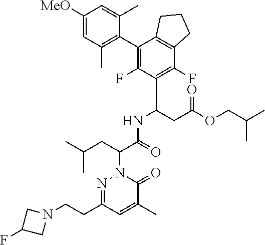 [SMILES: COc1cc(C)c(-c2c(F)c(C(CC(=O)OCC(C)C)NC(=O)C(CC(C)C)n3nc(CCN4CC(F)C4)cc(C)c3=O)c(F)c3c2CCC3)c(C)c1] | 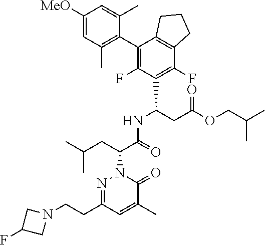 [SMILES: COc1cc(C)c(-c2c(F)c([C@H](CC(=O)OCC(C)C)NC(=O)[C@@H](CC(C)C)n3nc(CCN4CC(F)C4)cc(C)c3=O)c(F)c3c2CCC3)c(C)c1] |
| 418 | 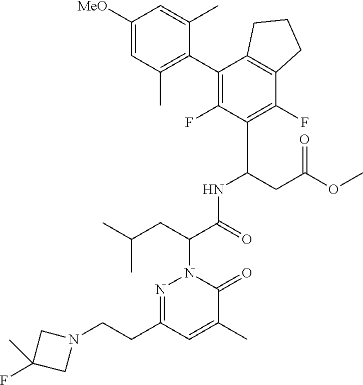 [SMILES: COC(=O)CC(NC(=O)C(CC(C)C)n1nc(CCN2CC(C)(F)C2)cc(C)c1=O)c1c(F)c2c(c(-c3c(C)cc(OC)cc3C)c1F)CCC2] | 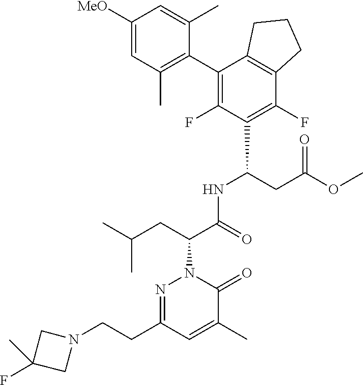 [SMILES: COC(=O)C[C@H](NC(=O)[C@@H](CC(C)C)n1nc(CCN2CC(C)(F)C2)cc(C)c1=O)c1c(F)c2c(c(-c3c(C)cc(OC)cc3C)c1F)CCC2] |
| 419 | 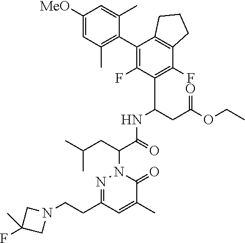 [SMILES: CCOC(=O)CC(NC(=O)C(CC(C)C)n1nc(CCN2CC(C)(F)C2)cc(C)c1=O)c1c(F)c2c(c(-c3c(C)cc(OC)cc3C)c1F)CCC2] | 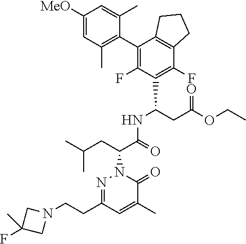 [SMILES: CCOC(=O)C[C@H](NC(=O)[C@@H](CC(C)C)n1nc(CCN2CC(C)(F)C2)cc(C)c1=O)c1c(F)c2c(c(-c3c(C)cc(OC)cc3C)c1F)CCC2] |
| 420 | 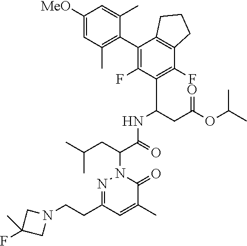 [SMILES: COc1cc(C)c(-c2c(F)c(C(CC(=O)OC(C)C)NC(=O)C(CC(C)C)n3nc(CCN4CC(C)(F)C4)cc(C)c3=O)c(F)c3c2CCC3)c(C)c1] | 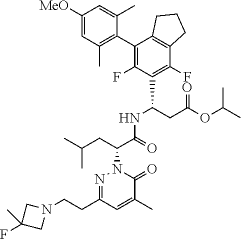 [SMILES: COc1cc(C)c(-c2c(F)c([C@H](CC(=O)OC(C)C)NC(=O)[C@@H](CC(C)C)n3nc(CCN4CC(C)(F)C4)cc(C)c3=O)c(F)c3c2CCC3)c(C)c1] |
| 421 | 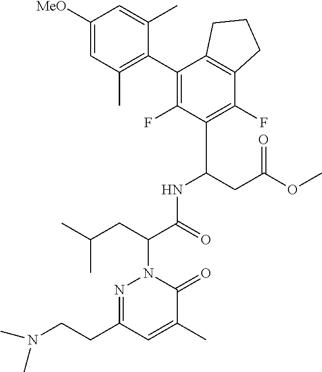 | 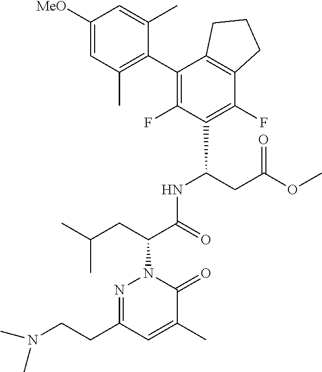 |
| 422 | 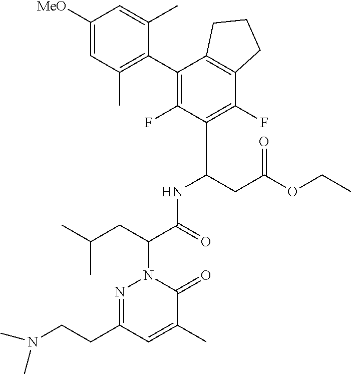 [SMILES: CCOC(=O)CC(NC(=O)C(CC(C)C)n1nc(CCN(C)C)cc(C)c1=O)c1c(F)c2c(c(-c3c(C)cc(OC)cc3C)c1F)CCC2] | 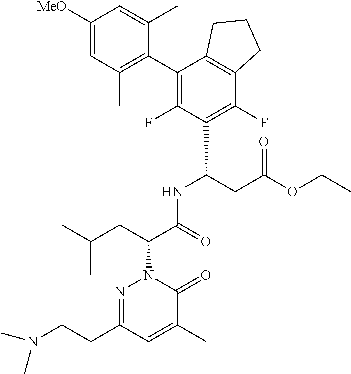 [SMILES: CCOC(=O)C[C@H](NC(=O)[C@@H](CC(C)C)n1nc(CCN(C)C)cc(C)c1=O)c1c(F)c2c(c(-c3c(C)cc(OC)cc3C)c1F)CCC2] |
| 423 | 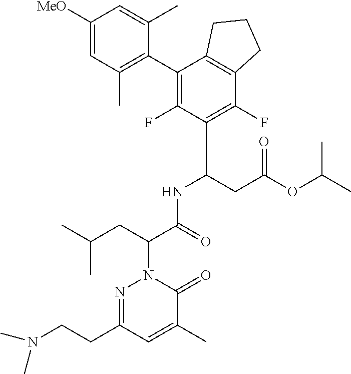 [SMILES: COc1cc(C)c(-c2c(F)c(C(CC(=O)OC(C)C)NC(=O)C(CC(C)C)n3nc(CCN(C)C)cc(C)c3=O)c(F)c3c2CCC3)c(C)c1] | 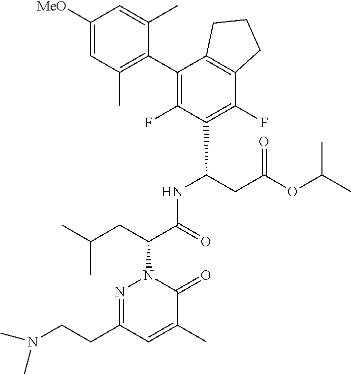 [SMILES: COc1cc(C)c(-c2c(F)c([C@H](CC(=O)OC(C)C)NC(=O)[C@@H](CC(C)C)n3nc(CCN(C)C)cc(C)c3=O)c(F)c3c2CCC3)c(C)c1] |
| 424 | 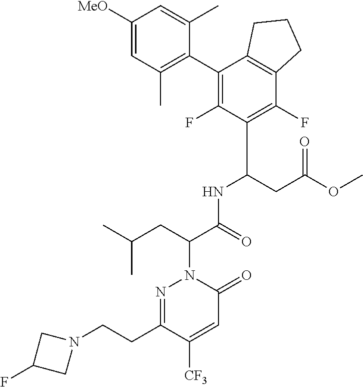 [SMILES: COC(=O)CC(NC(=O)C(CC(C)C)n1nc(CCN2CC(F)C2)c(C(F)(F)F)cc1=O)c1c(F)c2c(c(-c3c(C)cc(OC)cc3C)c1F)CCC2] | 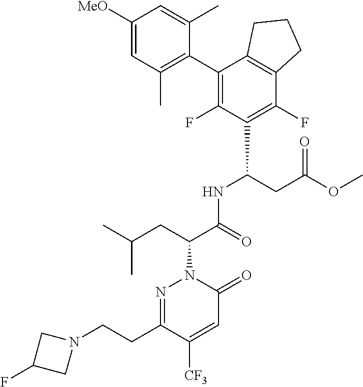 [SMILES: COC(=O)C[C@H](NC(=O)[C@@H](CC(C)C)n1nc(CCN2CC(F)C2)c(C(F)(F)F)cc1=O)c1c(F)c2c(c(-c3c(C)cc(OC)cc3C)c1F)CCC2] |
| 425 | 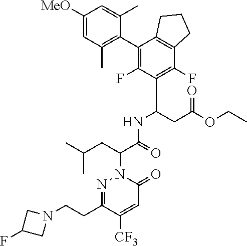 [SMILES: CCOC(=O)CC(NC(=O)C(CC(C)C)n1nc(CCN2CC(F)C2)c(C(F)(F)F)cc1=O)c1c(F)c2c(c(-c3c(C)cc(OC)cc3C)c1F)CCC2] | 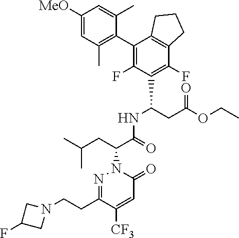 [SMILES: CCOC(=O)C[C@H](NC(=O)[C@@H](CC(C)C)n1nc(CCN2CC(F)C2)c(C(F)(F)F)cc1=O)c1c(F)c2c(c(-c3c(C)cc(OC)cc3C)c1F)CCC2] |
| 426 | 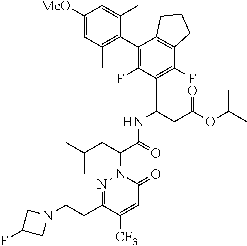 [SMILES: COc1cc(C)c(-c2c(F)c(C(CC(=O)OC(C)C)NC(=O)C(CC(C)C)n3nc(CCN4CC(F)C4)c(C(F)(F)F)cc3=O)c(F)c3c2CCC3)c(C)c1] | 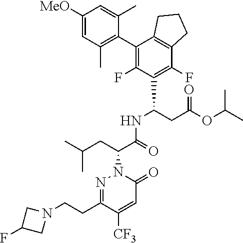 [SMILES: COc1cc(C)c(-c2c(F)c([C@H](CC(=O)OC(C)C)NC(=O)[C@@H](CC(C)C)n3nc(CCN4CC(F)C4)c(C(F)(F)F)cc3=O)c(F)c3c2CCC3)c(C)c1] |
| 427 | 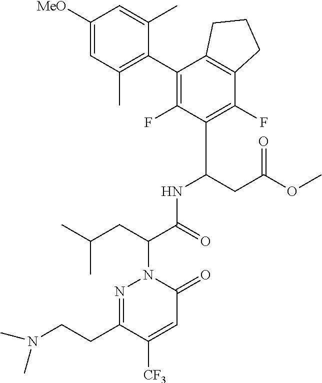 | 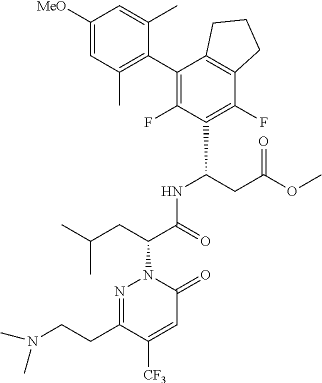 |
| 428 | 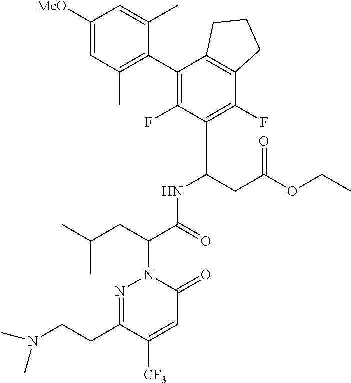 [SMILES: CCOC(=O)CC(NC(=O)C(CC(C)C)n1nc(CCN(C)C)c(C(F)(F)F)cc1=O)c1c(F)c2c(c(-c3c(C)cc(OC)cc3C)c1F)CCC2] | 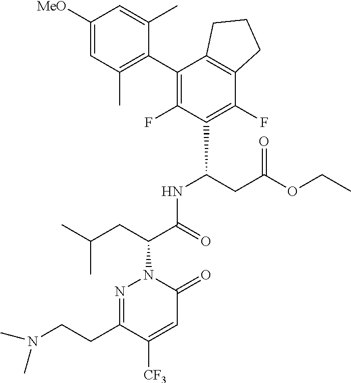 [SMILES: CCOC(=O)C[C@H](NC(=O)[C@@H](CC(C)C)n1nc(CCN(C)C)c(C(F)(F)F)cc1=O)c1c(F)c2c(c(-c3c(C)cc(OC)cc3C)c1F)CCC2] |
| 429 | 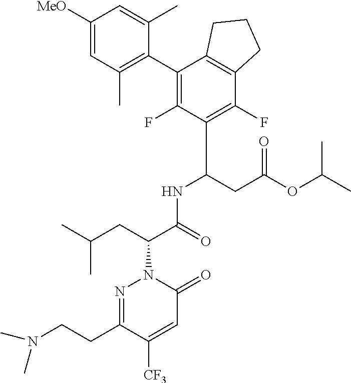 [SMILES: COc1cc(C)c(-c2c(F)c(C(CC(=O)OC(C)C)NC(=O)[C@@H](CC(C)C)n3nc(CCN(C)C)c(C(F)(F)F)cc3=O)c(F)c3c2CCC3)c(C)c1] | 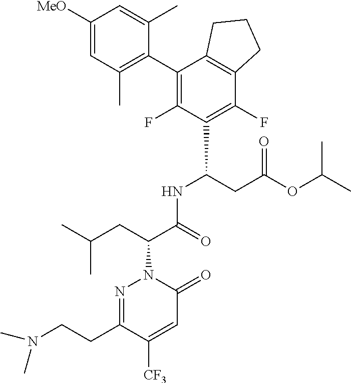 [SMILES: COc1cc(C)c(-c2c(F)c([C@H](CC(=O)OC(C)C)NC(=O)[C@@H](CC(C)C)n3nc(CCN(C)C)c(C(F)(F)F)cc3=O)c(F)c3c2CCC3)c(C)c1] |
| 430 | 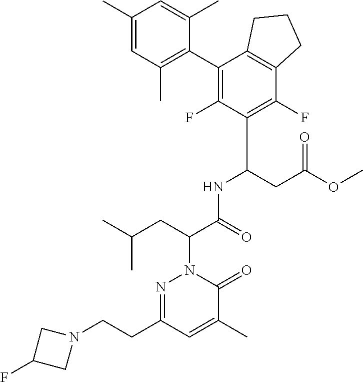 [SMILES: COC(=O)CC(NC(=O)C(CC(C)C)n1nc(CCN2CC(F)C2)cc(C)c1=O)c1c(F)c2c(c(-c3c(C)cc(C)cc3C)c1F)CCC2] | 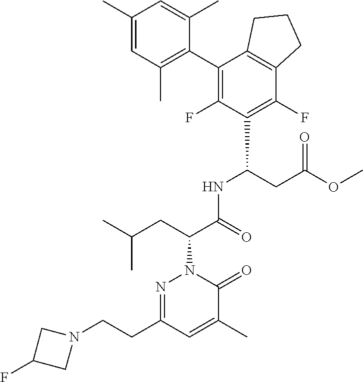 [SMILES: COC(=O)C[C@H](NC(=O)[C@@H](CC(C)C)n1nc(CCN2CC(F)C2)cc(C)c1=O)c1c(F)c2c(c(-c3c(C)cc(C)cc3C)c1F)CCC2] |
| 431 | 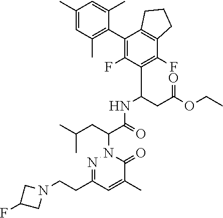 [SMILES: CCOC(=O)CC(NC(=O)C(CC(C)C)n1nc(CCN2CC(F)C2)cc(C)c1=O)c1c(F)c2c(c(-c3c(C)cc(C)cc3C)c1F)CCC2] | 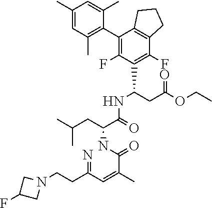 [SMILES: CCOC(=O)C[C@H](NC(=O)[C@@H](CC(C)C)n1nc(CCN2CC(F)C2)cc(C)c1=O)c1c(F)c2c(c(-c3c(C)cc(C)cc3C)c1F)CCC2] |
| 432 | 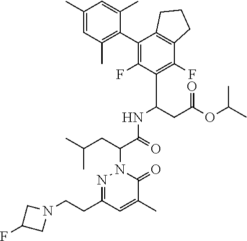 [SMILES: Cc1cc(C)c(-c2c(F)c(C(CC(=O)OC(C)C)NC(=O)C(CC(C)C)n3nc(CCN4CC(F)C4)cc(C)c3=O)c(F)c3c2CCC3)c(C)c1] | 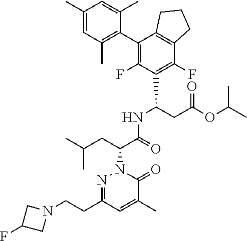 [SMILES: Cc1cc(C)c(-c2c(F)c([C@H](CC(=O)OC(C)C)NC(=O)[C@@H](CC(C)C)n3nc(CCN4CC(F)C4)cc(C)c3=O)c(F)c3c2CCC3)c(C)c1] |
| 433 | 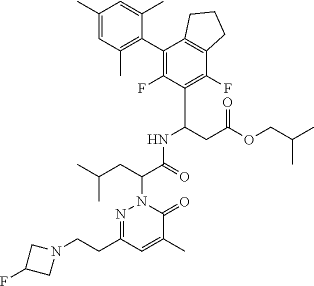 [SMILES: Cc1cc(C)c(-c2c(F)c(C(CC(=O)OCC(C)C)NC(=O)C(CC(C)C)n3nc(CCN4CC(F)C4)cc(C)c3=O)c(F)c3c2CCC3)c(C)c1] | 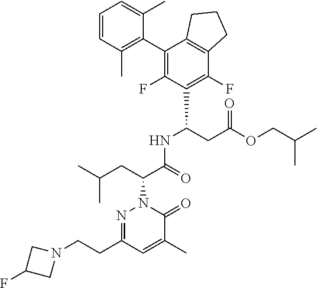 [SMILES: Cc1cccc(C)c1-c1c(F)c([C@H](CC(=O)OCC(C)C)NC(=O)[C@@H](CC(C)C)n2nc(CCN3CC(F)C3)cc(C)c2=O)c(F)c2c1CCC2] |
| 434 | 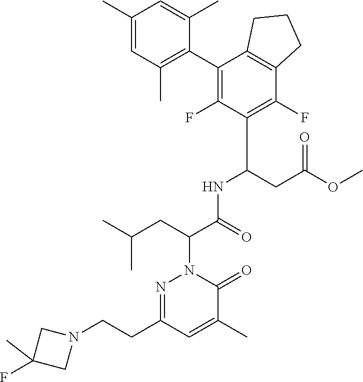 [SMILES: COC(=O)CC(NC(=O)C(CC(C)C)n1nc(CCN2CC(C)(F)C2)cc(C)c1=O)c1c(F)c2c(c(-c3c(C)cc(C)cc3C)c1F)CCC2] | 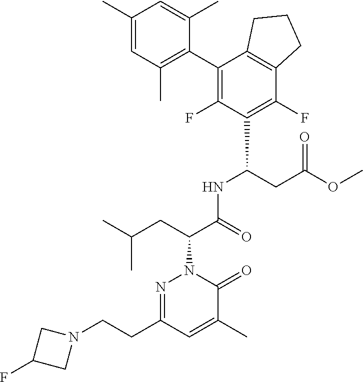 [SMILES: COC(=O)C[C@H](NC(=O)[C@@H](CC(C)C)n1nc(CCN2CC(F)C2)cc(C)c1=O)c1c(F)c2c(c(-c3c(C)cc(C)cc3C)c1F)CCC2] |
| 435 | 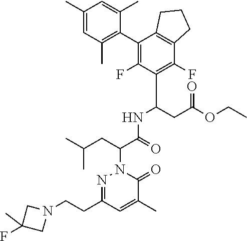 [SMILES: CCOC(=O)CC(NC(=O)C(CC(C)C)n1nc(CCN2CC(C)(F)C2)cc(C)c1=O)c1c(F)c2c(c(-c3c(C)cc(C)cc3C)c1F)CCC2] | 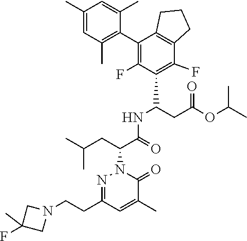 |
| 436 | 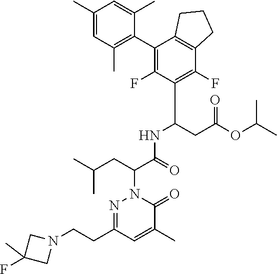 [SMILES: Cc1cc(C)c(-c2c(F)c(C(CC(=O)OC(C)C)NC(=O)C(CC(C)C)n3nc(CCN4CC(C)(F)C4)cc(C)c3=O)c(F)c3c2CCC3)c(C)c1] | 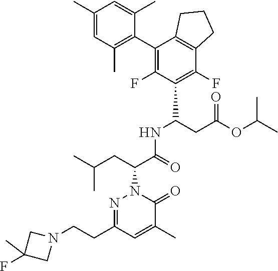 |
| 437 | 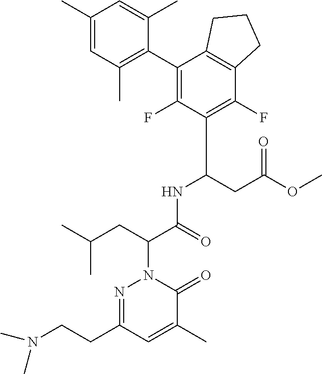 | 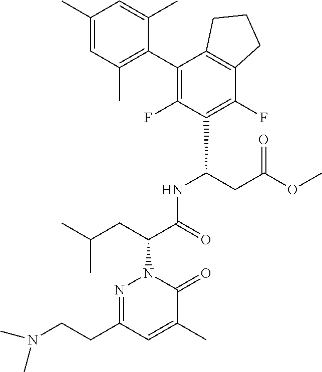 |
| 438 | 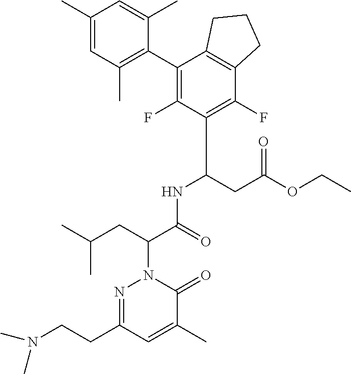 [SMILES: CCOC(=O)CC(NC(=O)C(CC(C)C)n1nc(CCN(C)C)cc(C)c1=O)c1c(F)c2c(c(-c3c(C)cc(C)cc3C)c1F)CCC2] | 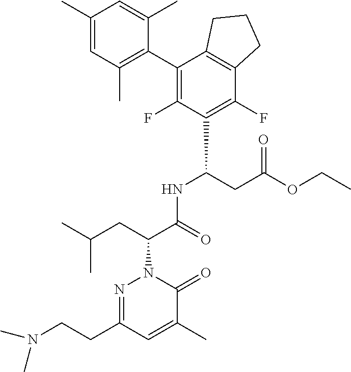 [SMILES: CCOC(=O)C[C@H](NC(=O)[C@@H](CC(C)C)n1nc(CCN(C)C)cc(C)c1=O)c1c(F)c2c(c(-c3c(C)cc(C)cc3C)c1F)CCC2] |
| 439 | 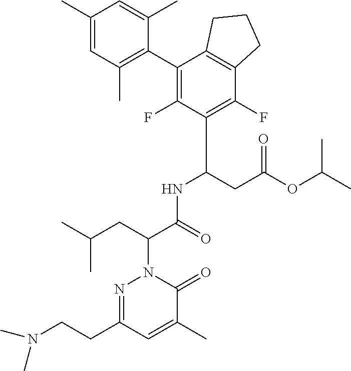 [SMILES: Cc1cc(C)c(-c2c(F)c(C(CC(=O)OC(C)C)NC(=O)C(CC(C)C)n3nc(CCN(C)C)cc(C)c3=O)c(F)c3c2CCC3)c(C)c1] | 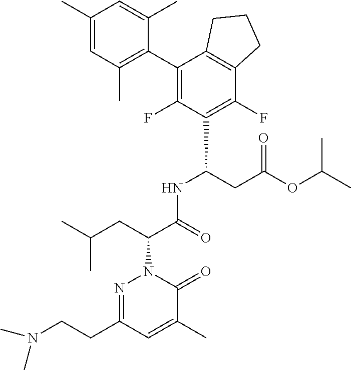 [SMILES: Cc1cc(C)c(-c2c(F)c([C@H](CC(=O)OC(C)C)NC(=O)[C@@H](CC(C)C)n3nc(CCN(C)C)cc(C)c3=O)c(F)c3c2CCC3)c(C)c1] |
| 440 | 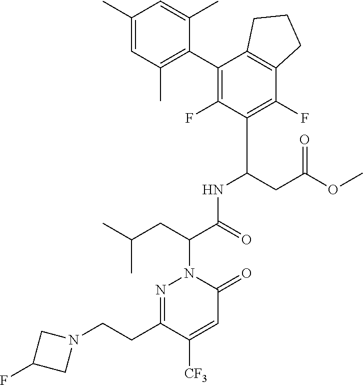 [SMILES: COC(=O)CC(NC(=O)C(CC(C)C)n1nc(CCN2CC(F)C2)c(C(F)(F)F)cc1=O)c1c(F)c2c(c(-c3c(C)cc(C)cc3C)c1F)CCC2] | 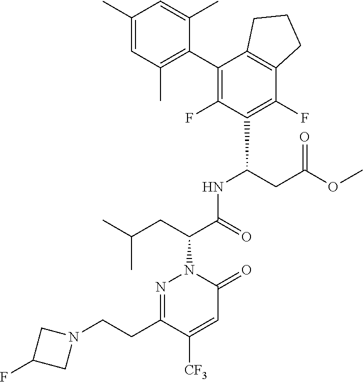 [SMILES: COC(=O)C[C@H](NC(=O)[C@@H](CC(C)C)n1nc(CCN2CC(F)C2)c(C(F)(F)F)cc1=O)c1c(F)c2c(c(-c3c(C)cc(C)cc3C)c1F)CCC2] |
| 441 | 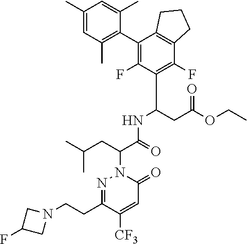 [SMILES: CCOC(=O)CC(NC(=O)C(CC(C)C)n1nc(CCN2CC(F)C2)c(C(F)(F)F)cc1=O)c1c(F)c2c(c(-c3c(C)cc(C)cc3C)c1F)CCC2] | 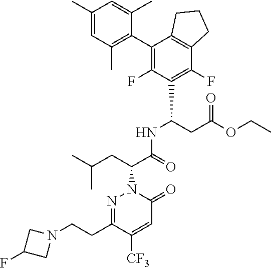 [SMILES: CCOC(=O)C[C@H](NC(=O)[C@@H](CC(C)C)n1nc(CCN2CC(F)C2)c(C(F)(F)F)cc1=O)c1c(F)c2c(c(-c3c(C)cc(C)cc3C)c1F)CCC2] |
| 442 | 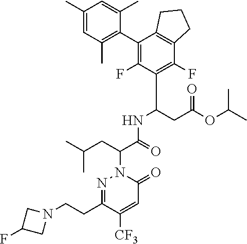 [SMILES: Cc1cc(C)c(-c2c(F)c(C(CC(=O)OC(C)C)NC(=O)C(CC(C)C)n3nc(CCN4CC(F)C4)c(C(F)(F)F)cc3=O)c(F)c3c2CCC3)c(C)c1] | 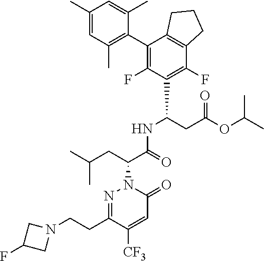 [SMILES: Cc1cc(C)c(-c2c(F)c([C@H](CC(=O)OC(C)C)NC(=O)[C@@H](CC(C)C)n3nc(CCN4CC(F)C4)c(C(F)(F)F)cc3=O)c(F)c3c2CCC3)c(C)c1] |
| 443 | 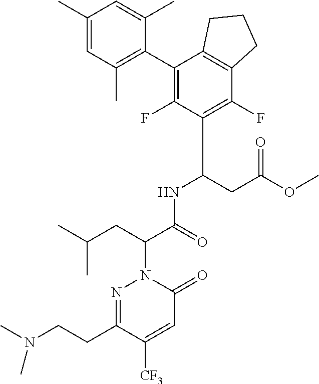 | 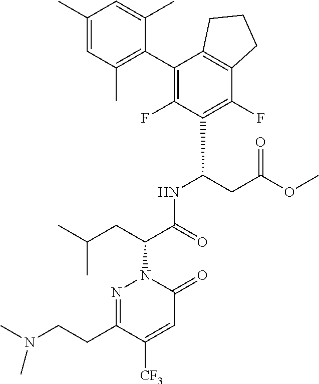 |
| 444 | 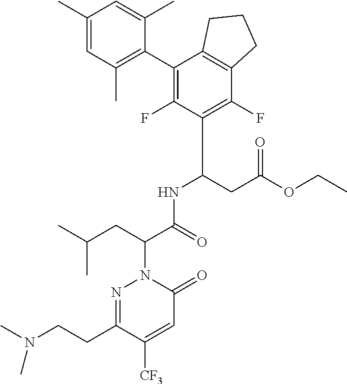 [SMILES: CCOC(=O)CC(NC(=O)C(CC(C)C)n1nc(CCN(C)C)c(C(F)(F)F)cc1=O)c1c(F)c2c(c(-c3c(C)cc(C)cc3C)c1F)CCC2] | 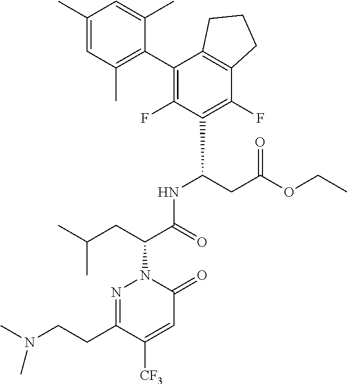 [SMILES: CCOC(=O)C[C@H](NC(=O)[C@@H](CC(C)C)n1nc(CCN(C)C)c(C(F)(F)F)cc1=O)c1c(F)c2c(c(-c3c(C)cc(C)cc3C)c1F)CCC2] |
| 445 | 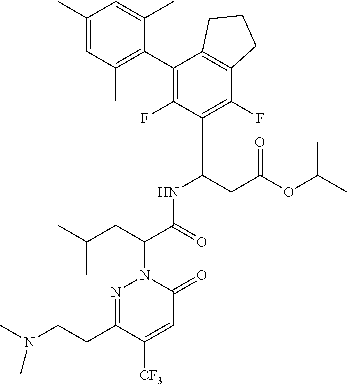 [SMILES: Cc1cc(C)c(-c2c(F)c(C(CC(=O)OC(C)C)NC(=O)C(CC(C)C)n3nc(CCN(C)C)c(C(F)(F)F)cc3=O)c(F)c3c2CCC3)c(C)c1] | 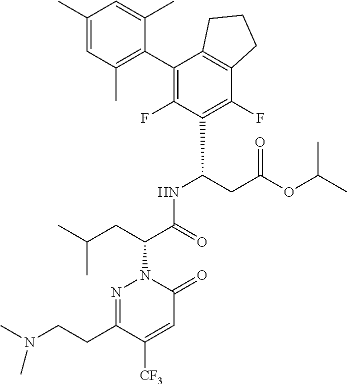 [SMILES: Cc1cc(C)c(-c2c(F)c([C@H](CC(=O)OC(C)C)NC(=O)[C@@H](CC(C)C)n3nc(CCN(C)C)c(C(F)(F)F)cc3=O)c(F)c3c2CCC3)c(C)c1] |
| 446 | 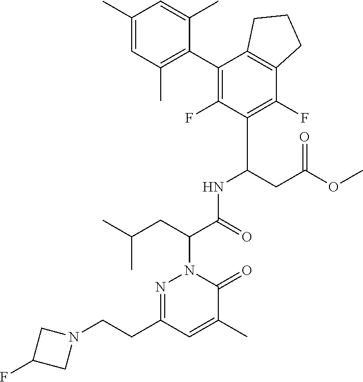 [SMILES: COC(=O)CC(NC(=O)C(CC(C)C)n1nc(CCN2CC(F)C2)cc(C)c1=O)c1c(F)c2c(c(-c3c(C)cc(C)cc3C)c1F)CCC2] | 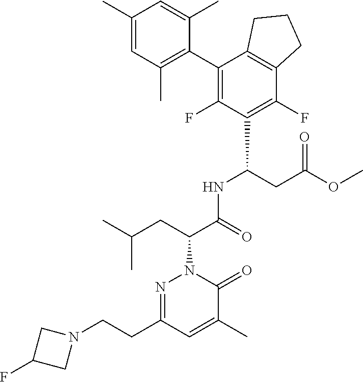 [SMILES: COC(=O)C[C@H](NC(=O)[C@@H](CC(C)C)n1nc(CCN2CC(F)C2)cc(C)c1=O)c1c(F)c2c(c(-c3c(C)cc(C)cc3C)c1F)CCC2] |
| 447 | 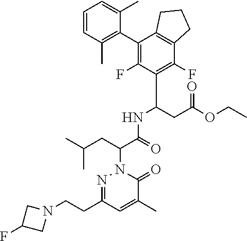 [SMILES: CCOC(=O)CC(NC(=O)C(CC(C)C)n1nc(CCN2CC(F)C2)cc(C)c1=O)c1c(F)c2c(c(-c3c(C)cccc3C)c1F)CCC2] | 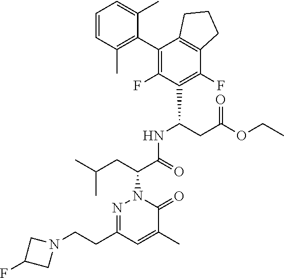 [SMILES: CCOC(=O)C[C@H](NC(=O)[C@@H](CC(C)C)n1nc(CCN2CC(F)C2)cc(C)c1=O)c1c(F)c2c(c(-c3c(C)cccc3C)c1F)CCC2] |
| 448 | 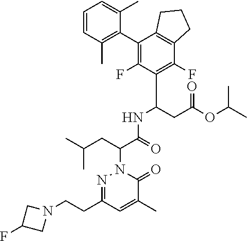 [SMILES: Cc1cccc(C)c1-c1c(F)c(C(CC(=O)OC(C)C)NC(=O)C(CC(C)C)n2nc(CCN3CC(F)C3)cc(C)c2=O)c(F)c2c1CCC2] | 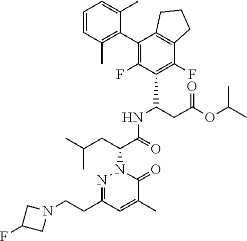 |
| 449 | 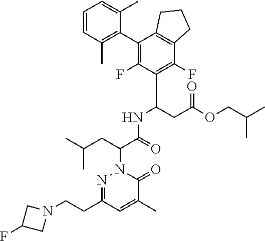 [SMILES: Cc1cccc(C)c1-c1c(F)c(C(CC(=O)OCC(C)C)NC(=O)C(CC(C)C)n2nc(CCN3CC(F)C3)cc(C)c2=O)c(F)c2c1CCC2] | 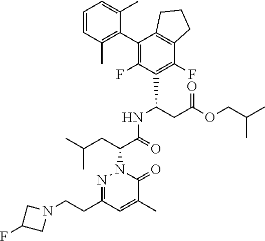 [SMILES: Cc1cccc(C)c1-c1c(F)c([C@H](CC(=O)OCC(C)C)NC(=O)[C@@H](CC(C)C)n2nc(CCN3CC(F)C3)cc(C)c2=O)c(F)c2c1CCC2] |
| 450 | 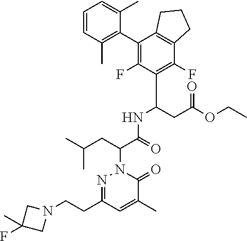 [SMILES: CCOC(=O)CC(NC(=O)C(CC(C)C)n1nc(CCN2CC(C)(F)C2)cc(C)c1=O)c1c(F)c2c(c(-c3c(C)cccc3C)c1F)CCC2] | 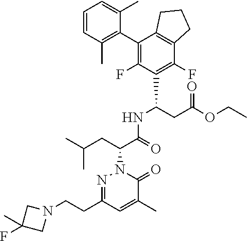 [SMILES: CCOC(=O)C[C@H](NC(=O)[C@@H](CC(C)C)n1nc(CCN2CC(C)(F)C2)cc(C)c1=O)c1c(F)c2c(c(-c3c(C)cccc3C)c1F)CCC2] |
| 451 | 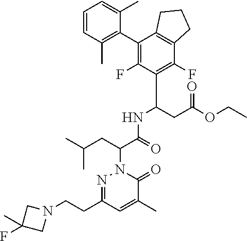 [SMILES: CCOC(=O)CC(NC(=O)C(CC(C)C)n1nc(CCN2CC(C)(F)C2)cc(C)c1=O)c1c(F)c2c(c(-c3c(C)cccc3C)c1F)CCC2] | 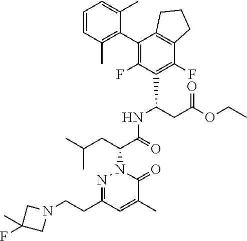 [SMILES: CCOC(=O)C[C@H](NC(=O)[C@@H](CC(C)C)n1nc(CCN2CC(C)(F)C2)cc(C)c1=O)c1c(F)c2c(c(-c3c(C)cccc3C)c1F)CCC2] |
| 452 | 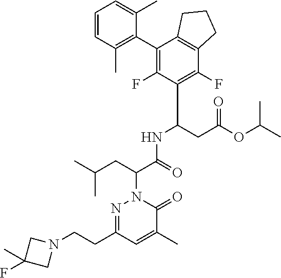 [SMILES: Cc1cccc(C)c1-c1c(F)c(C(CC(=O)OC(C)C)NC(=O)C(CC(C)C)n2nc(CCN3CC(C)(F)C3)cc(C)c2=O)c(F)c2c1CCC2] | 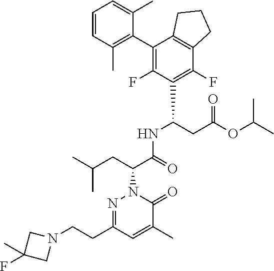 [SMILES: Cc1cccc(C)c1-c1c(F)c([C@H](CC(=O)OC(C)C)NC(=O)[C@@H](CC(C)C)n2nc(CCN3CC(C)(F)C3)cc(C)c2=O)c(F)c2c1CCC2] |
| 453 | 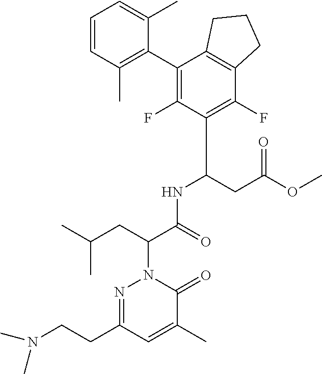 | 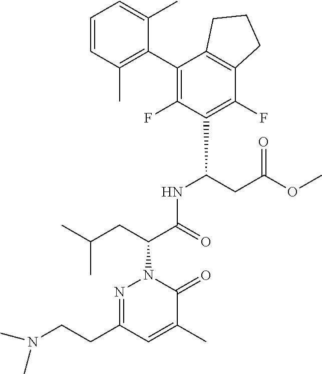 |
| 454 | 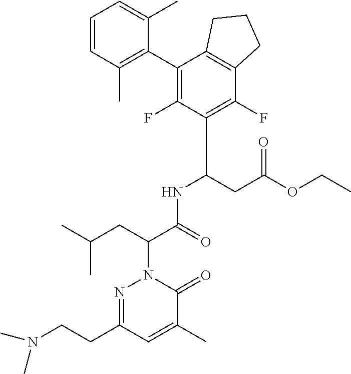 [SMILES: CCOC(=O)CC(NC(=O)C(CC(C)C)n1nc(CCN(C)C)cc(C)c1=O)c1c(F)c2c(c(-c3c(C)cccc3C)c1F)CCC2] | 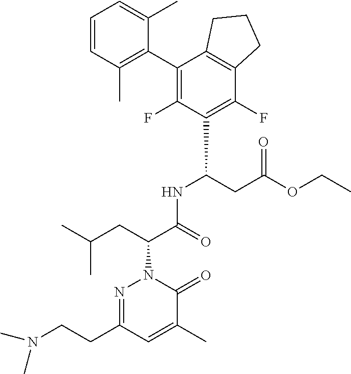 [SMILES: CCOC(=O)C[C@H](NC(=O)[C@@H](CC(C)C)n1nc(CCN(C)C)cc(C)c1=O)c1c(F)c2c(c(-c3c(C)cccc3C)c1F)CCC2] |
| 455 | 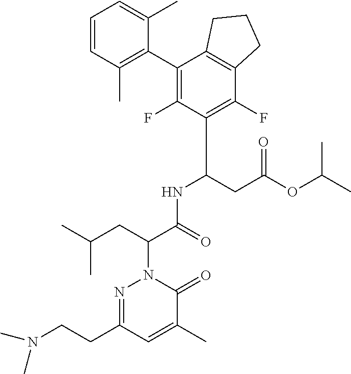 [SMILES: Cc1cccc(C)c1-c1c(F)c(C(CC(=O)OC(C)C)NC(=O)C(CC(C)C)n2nc(CCN(C)C)cc(C)c2=O)c(F)c2c1CCC2] | 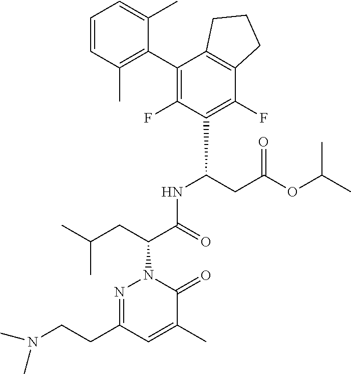 [SMILES: Cc1cccc(C)c1-c1c(F)c([C@H](CC(=O)OC(C)C)NC(=O)[C@@H](CC(C)C)n2nc(CCN(C)C)cc(C)c2=O)c(F)c2c1CCC2] |
| 456 | 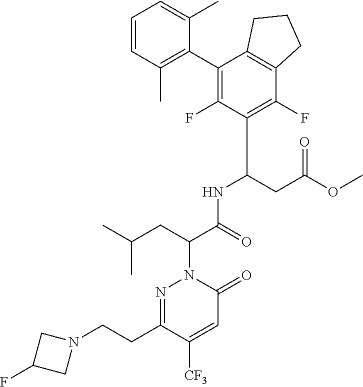 [SMILES: COC(=O)CC(NC(=O)C(CC(C)C)n1nc(CCN2CC(F)C2)c(C(F)(F)F)cc1=O)c1c(F)c2c(c(-c3c(C)cccc3C)c1F)CCC2] | 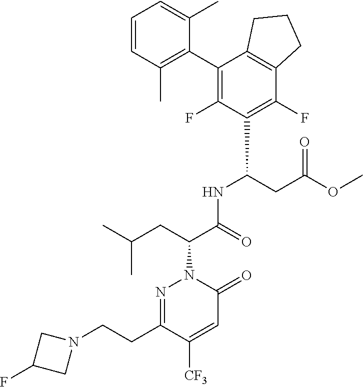 [SMILES: COC(=O)C[C@H](NC(=O)[C@@H](CC(C)C)n1nc(CCN2CC(F)C2)c(C(F)(F)F)cc1=O)c1c(F)c2c(c(-c3c(C)cccc3C)c1F)CCC2] |
| 457 | 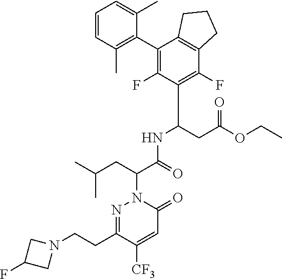 [SMILES: CCOC(=O)CC(NC(=O)C(CC(C)C)n1nc(CCN2CC(F)C2)c(C(F)(F)F)cc1=O)c1c(F)c2c(c(-c3c(C)cccc3C)c1F)CCC2] | 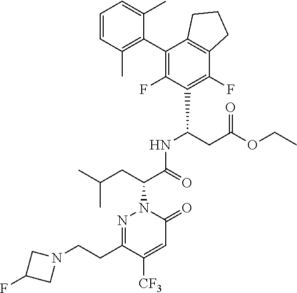 [SMILES: CCOC(=O)C[C@H](NC(=O)[C@@H](CC(C)C)n1nc(CCN2CC(F)C2)c(C(F)(F)F)cc1=O)c1c(F)c2c(c(-c3c(C)cccc3C)c1F)CCC2] |
| 458 | 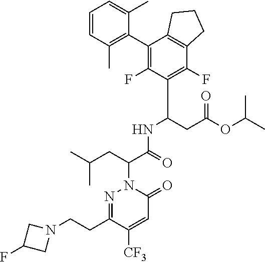 [SMILES: Cc1cccc(C)c1-c1c(F)c(C(CC(=O)OC(C)C)NC(=O)C(CC(C)C)n2nc(CCN3CC(F)C3)c(C(F)(F)F)cc2=O)c(F)c2c1CCC2] | 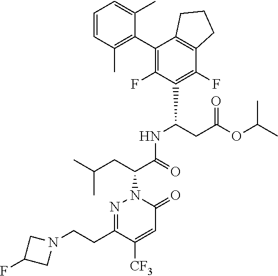 [SMILES: Cc1cccc(C)c1-c1c(F)c([C@H](CC(=O)OC(C)C)NC(=O)[C@@H](CC(C)C)n2nc(CCN3CC(F)C3)c(C(F)(F)F)cc2=O)c(F)c2c1CCC2] |
| 459 | 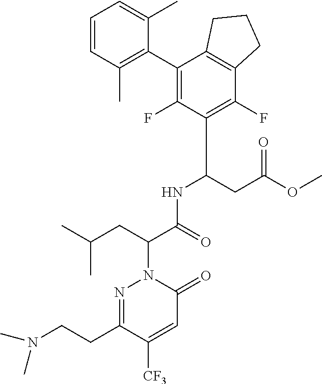 | 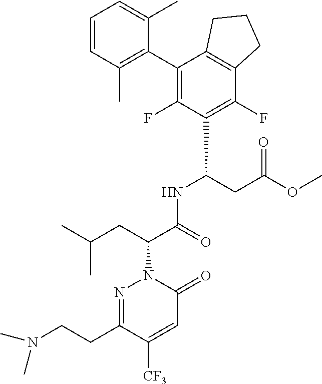 |
| 460 | 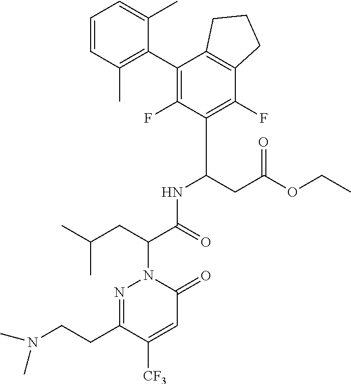 [SMILES: CCOC(=O)CC(NC(=O)C(CC(C)C)n1nc(CCN(C)C)c(C(F)(F)F)cc1=O)c1c(F)c2c(c(-c3c(C)cccc3C)c1F)CCC2] | 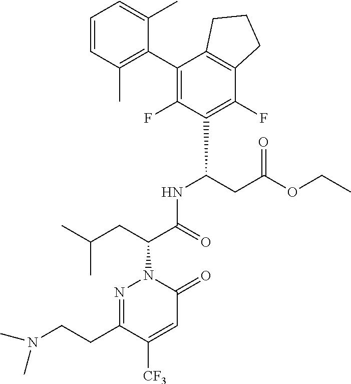 [SMILES: CCOC(=O)C[C@H](NC(=O)[C@@H](CC(C)C)n1nc(CCN(C)C)c(C(F)(F)F)cc1=O)c1c(F)c2c(c(-c3c(C)cccc3C)c1F)CCC2] |
| 461 | 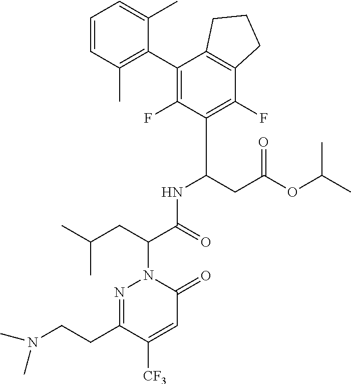 [SMILES: Cc1cccc(C)c1-c1c(F)c(C(CC(=O)OC(C)C)NC(=O)C(CC(C)C)n2nc(CCN(C)C)c(C(F)(F)F)cc2=O)c(F)c2c1CCC2] | 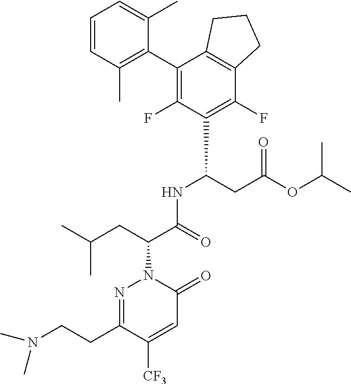 [SMILES: Cc1cccc(C)c1-c1c(F)c([C@H](CC(=O)OC(C)C)NC(=O)[C@@H](CC(C)C)n2nc(CCN(C)C)c(C(F)(F)F)cc2=O)c(F)c2c1CCC2] |

| | | |
|---|---|---|
| 362 | 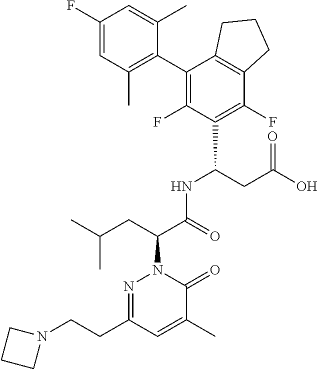 [SMILES: Cc1cc(F)cc(C)c1-c1c(F)c([C@H](CC(=O)O)NC(=O)[C@H](CC(C)C)n2nc(CCN3CCC3)cc(C)c2=O)c(F)c2c1CCC2] | 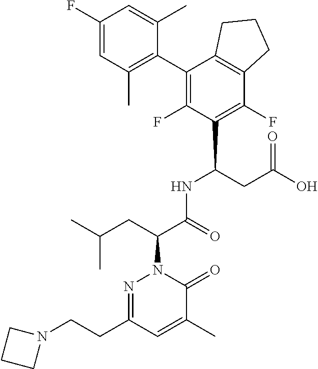 [SMILES: Cc1cc(F)cc(C)c1-c1c(F)c([C@@H](CC(=O)O)NC(=O)[C@H](CC(C)C)n2nc(CCN3CCC3)cc(C)c2=O)c(F)c2c1CCC2] |
| 363 | 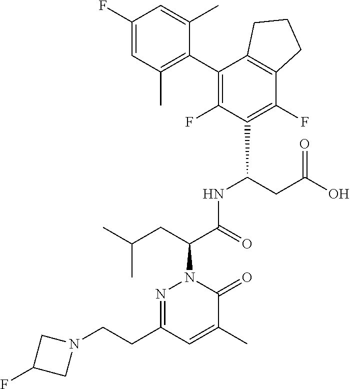 [SMILES: Cc1cc(F)cc(C)c1-c1c(F)c([C@H](CC(=O)O)NC(=O)[C@H](CC(C)C)n2nc(CCN3CC(F)C3)cc(C)c2=O)c(F)c2c1CCC2] | 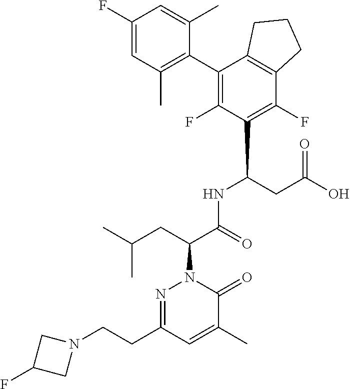 [SMILES: Cc1cc(F)cc(C)c1-c1c(F)c([C@@H](CC(=O)O)NC(=O)[C@H](CC(C)C)n2nc(CCN3CC(F)C3)cc(C)c2=O)c(F)c2c1CCC2] |
| 364 | 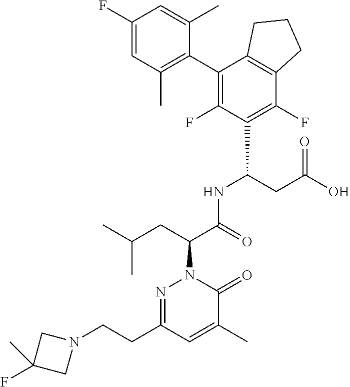 [SMILES: Cc1cc(F)cc(C)c1-c1c(F)c([C@H](CC(=O)O)NC(=O)[C@H](CC(C)C)n2nc(CCN3CC(C)(F)C3)cc(C)c2=O)c(F)c2c1CCC2] | 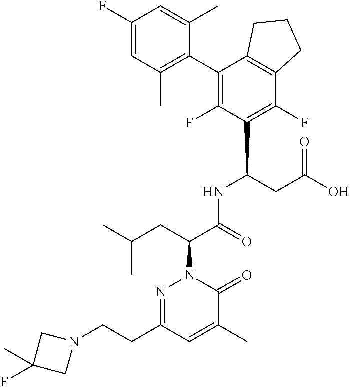 [SMILES: Cc1cc(F)cc(C)c1-c1c(F)c([C@@H](CC(=O)O)NC(=O)[C@H](CC(C)C)n2nc(CCN3CC(C)(F)C3)cc(C)c2=O)c(F)c2c1CCC2] |
| 365 | 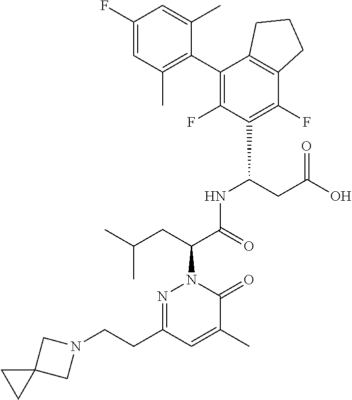 [SMILES: Cc1cc(F)cc(C)c1-c1c(F)c([C@H](CC(=O)O)NC(=O)[C@H](CC(C)C)n2nc(CCN3CC4(CC4)C3)cc(C)c2=O)c(F)c2c1CCC2] | 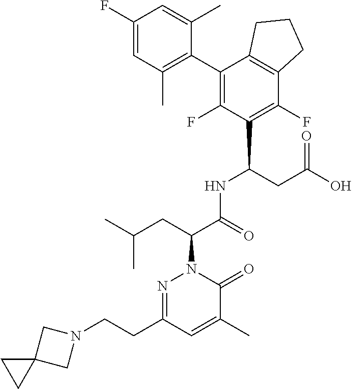 [SMILES: Cc1cc(F)cc(C)c1-c1c(F)c([C@@H](CC(=O)O)NC(=O)[C@H](CC(C)C)n2nc(CCN3CC4(CC4)C3)cc(C)c2=O)c(F)c2c1CCC2] |
| 366 | 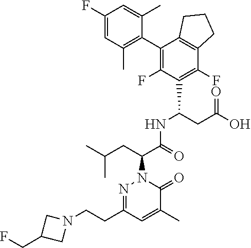 [SMILES: Cc1cc(F)cc(C)c1-c1c(F)c([C@H](CC(=O)O)NC(=O)[C@H](CC(C)C)n2nc(CCN3CC(CF)C3)cc(C)c2=O)c(F)c2c1CCC2] | 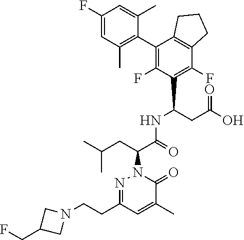 [SMILES: Cc1cc(F)cc(C)c1-c1c(F)c([C@@H](CC(=O)O)NC(=O)[C@H](CC(C)C)n2nc(CCN3CC(CF)C3)cc(C)c2=O)c(F)c2c1CCC2] |
| 367 | 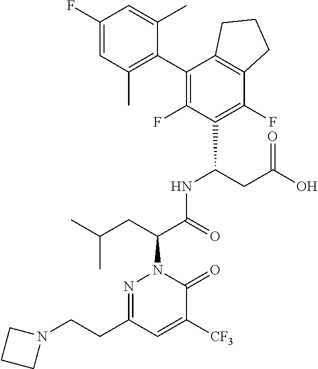 [SMILES: Cc1cc(F)cc(C)c1-c1c(F)c([C@H](CC(=O)O)NC(=O)[C@H](CC(C)C)n2nc(CCN3CCC3)cc(C(F)(F)F)c2=O)c(F)c2c1CCC2] | 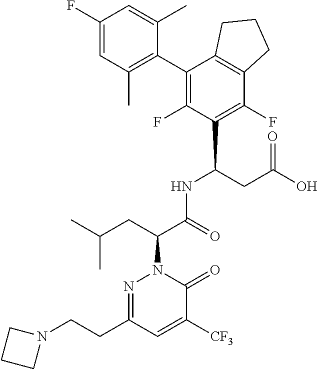 [SMILES: Cc1cc(F)cc(C)c1-c1c(F)c([C@@H](CC(=O)O)NC(=O)[C@H](CC(C)C)n2nc(CCN3CCC3)cc(C(F)(F)F)c2=O)c(F)c2c1CCC2] |
| 368 | 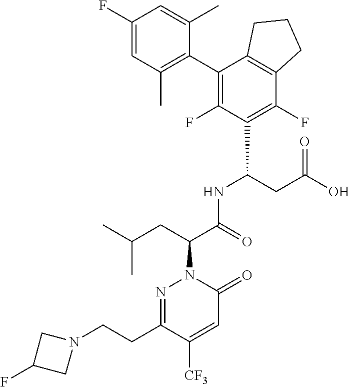 [SMILES: Cc1cc(F)cc(C)c1-c1c(F)c([C@H](CC(=O)O)NC(=O)[C@H](CC(C)C)n2nc(CCN3CC(F)C3)c(C(F)(F)F)cc2=O)c(F)c2c1CCC2] | 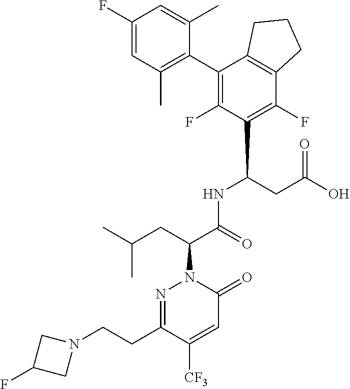 [SMILES: Cc1cc(F)cc(C)c1-c1c(F)c([C@@H](CC(=O)O)NC(=O)[C@H](CC(C)C)n2nc(CCN3CC(F)C3)c(C(F)(F)F)cc2=O)c(F)c2c1CCC2] |
| 369 | 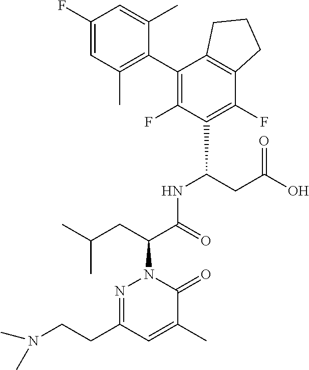 [SMILES: Cc1cc(F)cc(C)c1-c1c(F)c([C@H](CC(=O)O)NC(=O)[C@H](CC(C)C)n2nc(CCN(C)C)cc(C)c2=O)c(F)c2c1CCC2] | 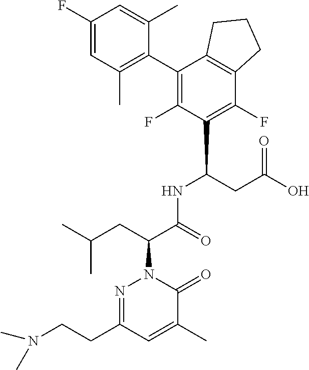 [SMILES: Cc1cc(F)cc(C)c1-c1c(F)c([C@@H](CC(=O)O)NC(=O)[C@H](CC(C)C)n2nc(CCN(C)C)cc(C)c2=O)c(F)c2c1CCC2] |
| 370 | 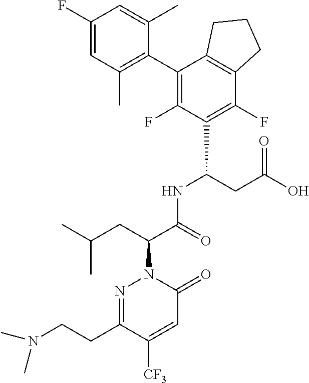 [SMILES: Cc1cc(F)cc(C)c1-c1c(F)c([C@H](CC(=O)O)NC(=O)[C@H](CC(C)C)n2nc(CCN(C)C)c(C(F)(F)F)cc2=O)c(F)c2c1CCC2] | 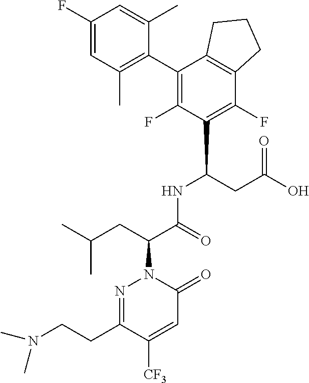 [SMILES: Cc1cc(F)cc(C)c1-c1c(F)c([C@@H](CC(=O)O)NC(=O)[C@H](CC(C)C)n2nc(CCN(C)C)c(C(F)(F)F)cc2=O)c(F)c2c1CCC2] |
| 371 | 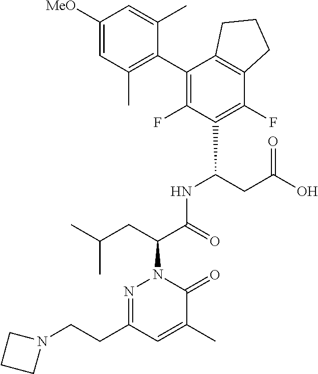 [SMILES: COc1cc(C)c(-c2c(F)c([C@H](CC(=O)O)NC(=O)[C@H](CC(C)C)n3nc(CCN4CCC4)cc(C)c3=O)c(F)c3c2CCC3)c(C)c1] | 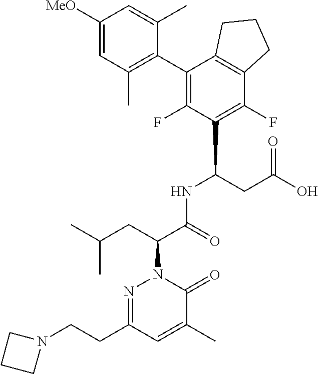 [SMILES: COc1cc(C)c(-c2c(F)c([C@@H](CC(=O)O)NC(=O)[C@H](CC(C)C)n3nc(CCN4CCC4)cc(C)c3=O)c(F)c3c2CCC3)c(C)c1] |
| 372 | 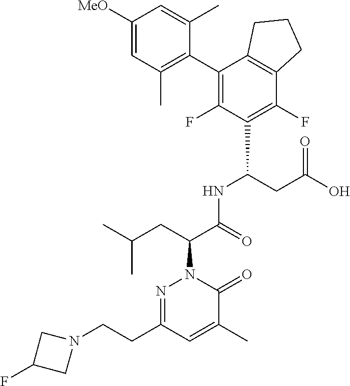 [SMILES: COc1cc(C)c(-c2c(F)c([C@H](CC(=O)O)NC(=O)[C@H](CC(C)C)n3nc(CCN4CC(F)C4)cc(C)c3=O)c(F)c3c2CCC3)c(C)c1] | 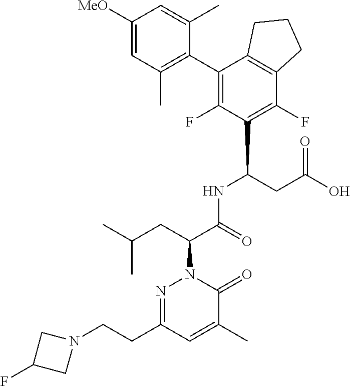 [SMILES: COc1cc(C)c(-c2c(F)c([C@@H](CC(=O)O)NC(=O)[C@H](CC(C)C)n3nc(CCN4CC(F)C4)cc(C)c3=O)c(F)c3c2CCC3)c(C)c1] |
| 373 | 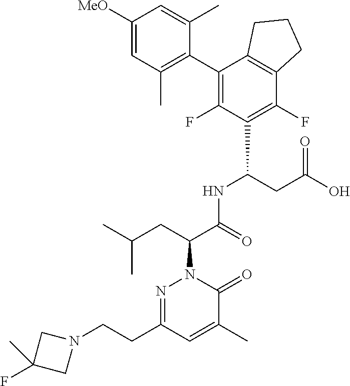 [SMILES: COc1cc(C)c(-c2c(F)c([C@H](CC(=O)O)NC(=O)[C@H](CC(C)C)n3nc(CCN4CC(C)(F)C4)cc(C)c3=O)c(F)c3c2CCC3)c(C)c1] | 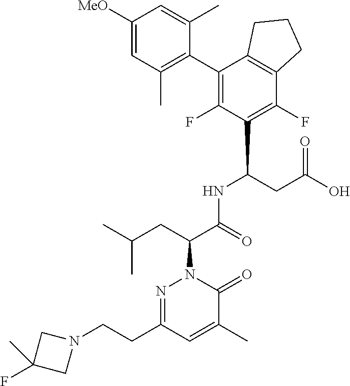 [SMILES: COc1cc(C)c(-c2c(F)c([C@@H](CC(=O)O)NC(=O)[C@H](CC(C)C)n3nc(CCN4CC(C)(F)C4)cc(C)c3=O)c(F)c3c2CCC3)c(C)c1] |
| 374 | 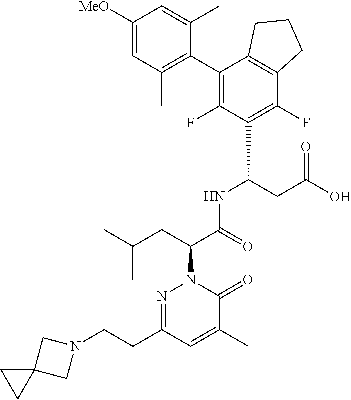 [SMILES: COc1cc(C)c(-c2c(F)c([C@H](CC(=O)O)NC(=O)[C@H](CC(C)C)n3nc(CCN4CC5(CC5)C4)cc(C)c3=O)c(F)c3c2CCC3)c(C)c1] | 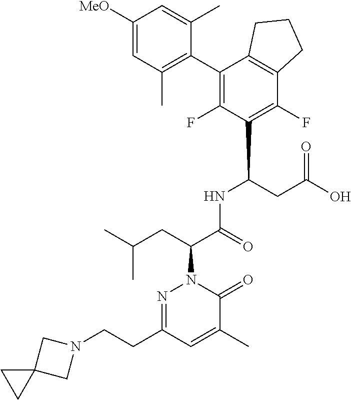 [SMILES: COc1cc(C)c(-c2c(F)c([C@@H](CC(=O)O)NC(=O)[C@H](CC(C)C)n3nc(CCN4CC5(CC5)C4)cc(C)c3=O)c(F)c3c2CCC3)c(C)c1] |
| 375 | 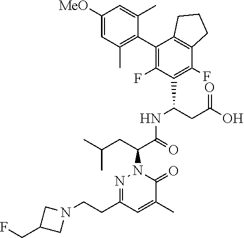 [SMILES: COc1cc(C)c(-c2c(F)c([C@H](CC(=O)O)NC(=O)[C@H](CC(C)C)n3nc(CCN4CC(CF)C4)cc(C)c3=O)c(F)c3c2CCC3)c(C)c1] | 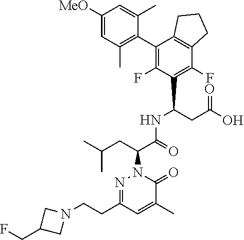 [SMILES: COc1cc(C)c(-c2c(F)c([C@@H](CC(=O)O)NC(=O)[C@H](CC(C)C)n3nc(CCN4CC(CF)C4)cc(C)c3=O)c(F)c3c2CCC3)c(C)c1] |
| 376 | 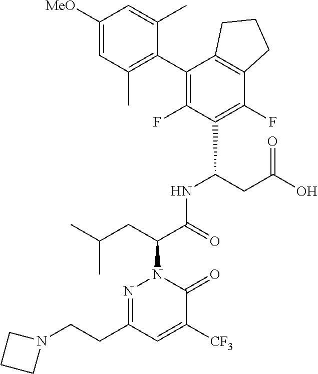 [SMILES: COc1cc(C)c(-c2c(F)c([C@H](CC(=O)O)NC(=O)[C@H](CC(C)C)n3nc(CCN4CCC4)cc(C(F)(F)F)c3=O)c(F)c3c2CCC3)c(C)c1] | 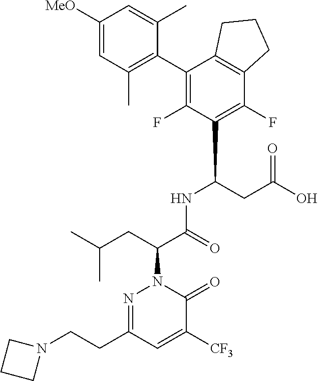 [SMILES: COc1cc(C)c(-c2c(F)c([C@@H](CC(=O)O)NC(=O)[C@H](CC(C)C)n3nc(CCN4CCC4)cc(C(F)(F)F)c3=O)c(F)c3c2CCC3)c(C)c1] |
| 377 | 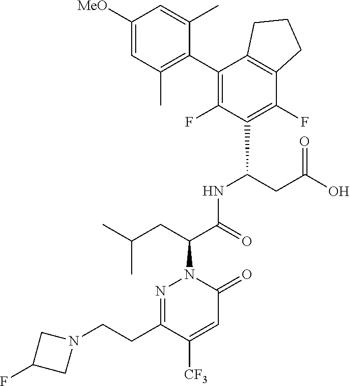 [SMILES: COc1cc(C)c(-c2c(F)c([C@H](CC(=O)O)NC(=O)[C@H](CC(C)C)n3nc(CCN4CC(F)C4)c(C(F)(F)F)cc3=O)c(F)c3c2CCC3)c(C)c1] | 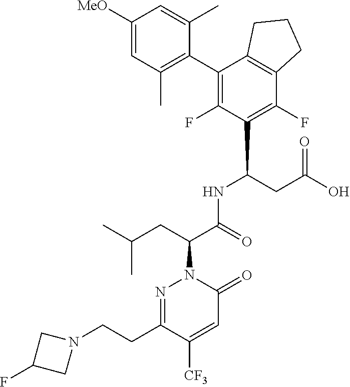 [SMILES: COc1cc(C)c(-c2c(F)c([C@@H](CC(=O)O)NC(=O)[C@H](CC(C)C)n3nc(CCN4CC(F)C4)c(C(F)(F)F)cc3=O)c(F)c3c2CCC3)c(C)c1] |
| 378 | 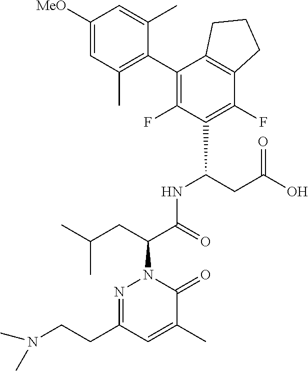 [SMILES: COc1cc(C)c(-c2c(F)c([C@H](CC(=O)O)NC(=O)[C@H](CC(C)C)n3nc(CCN(C)C)cc(C)c3=O)c(F)c3c2CCC3)c(C)c1] | 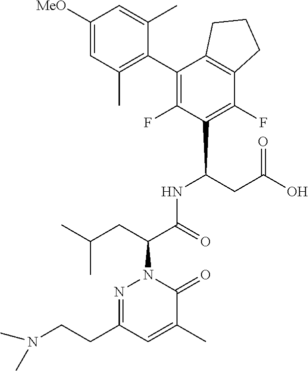 [SMILES: COc1cc(C)c(-c2c(F)c([C@@H](CC(=O)O)NC(=O)[C@H](CC(C)C)n3nc(CCN(C)C)cc(C)c3=O)c(F)c3c2CCC3)c(C)c1] |
| 379 | 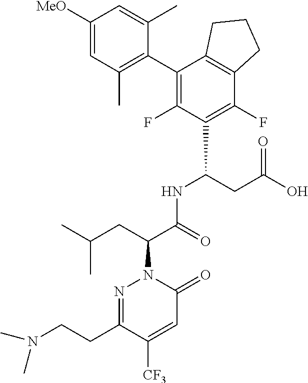 [SMILES: COc1cc(C)c(-c2c(F)c([C@H](CC(=O)O)NC(=O)[C@H](CC(C)C)n3nc(CCN(C)C)c(C(F)(F)F)cc3=O)c(F)c3c2CCC3)c(C)c1] | 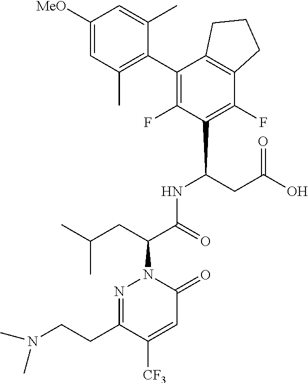 [SMILES: COc1cc(C)c(-c2c(F)c([C@@H](CC(=O)O)NC(=O)[C@H](CC(C)C)n3nc(CCN(C)C)c(C(F)(F)F)cc3=O)c(F)c3c2CCC3)c(C)c1] |
| 380 | 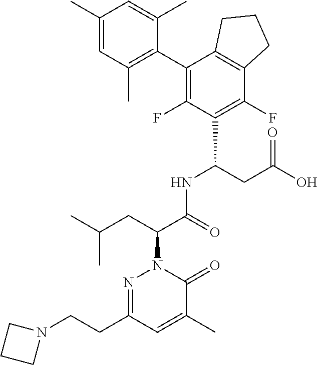 [SMILES: Cc1cc(C)c(-c2c(F)c([C@H](CC(=O)O)NC(=O)[C@H](CC(C)C)n3nc(CCN4CCC4)cc(C)c3=O)c(F)c3c2CCC3)c(C)c1] | 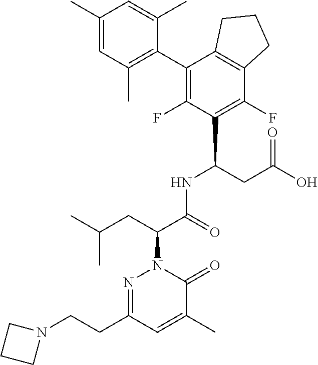 [SMILES: Cc1cc(C)c(-c2c(F)c([C@@H](CC(=O)O)NC(=O)[C@H](CC(C)C)n3nc(CCN4CCC4)cc(C)c3=O)c(F)c3c2CCC3)c(C)c1] |
| 381 | 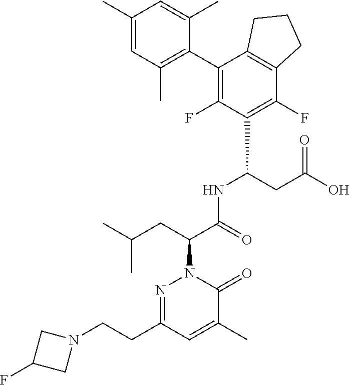 [SMILES: Cc1cc(C)c(-c2c(F)c([C@H](CC(=O)O)NC(=O)[C@H](CC(C)C)n3nc(CCN4CC(F)C4)cc(C)c3=O)c(F)c3c2CCC3)c(C)c1] | 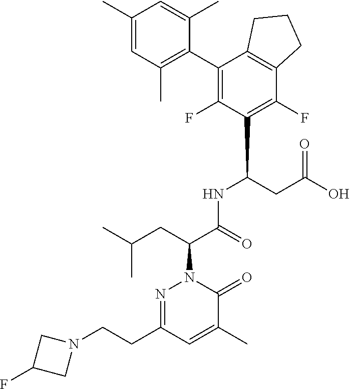 [SMILES: Cc1cc(C)c(-c2c(F)c([C@@H](CC(=O)O)NC(=O)[C@H](CC(C)C)n3nc(CCN4CC(F)C4)cc(C)c3=O)c(F)c3c2CCC3)c(C)c1] |
| 382 | 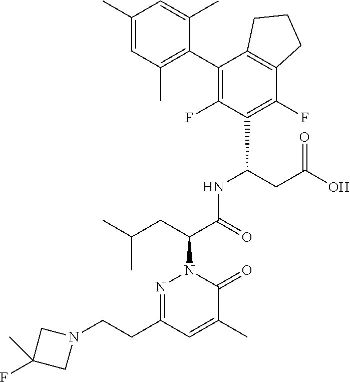 [SMILES: Cc1cc(C)c(-c2c(F)c([C@H](CC(=O)O)NC(=O)[C@H](CC(C)C)n3nc(CCN4CC(C)(F)C4)cc(C)c3=O)c(F)c3c2CCC3)c(C)c1] | 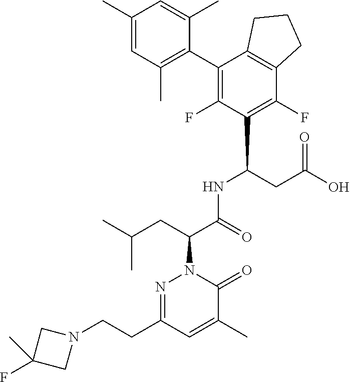 [SMILES: Cc1cc(C)c(-c2c(F)c([C@@H](CC(=O)O)NC(=O)[C@H](CC(C)C)n3nc(CCN4CC(C)(F)C4)cc(C)c3=O)c(F)c3c2CCC3)c(C)c1] |
| 383 | 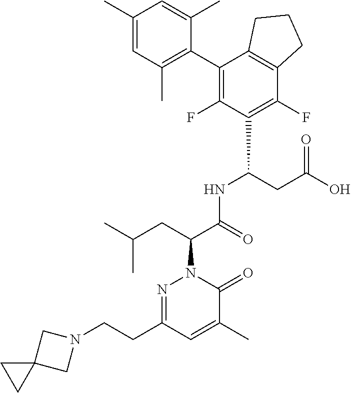 [SMILES: Cc1cc(C)c(-c2c(F)c([C@H](CC(=O)O)NC(=O)[C@H](CC(C)C)n3nc(CCN4CC5(CC5)C4)cc(C)c3=O)c(F)c3c2CCC3)c(C)c1] | 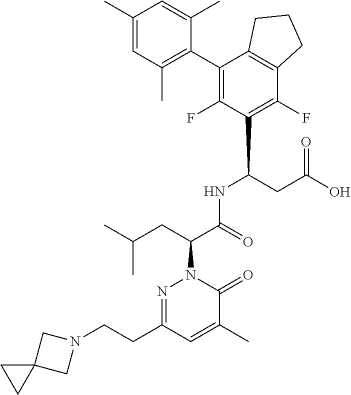 [SMILES: Cc1cc(C)c(-c2c(F)c([C@@H](CC(=O)O)NC(=O)[C@H](CC(C)C)n3nc(CCN4CC5(CC5)C4)cc(C)c3=O)c(F)c3c2CCC3)c(C)c1] |
| 384 | 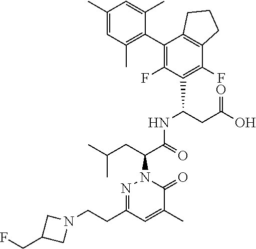 [SMILES: Cc1cc(C)c(-c2c(F)c([C@H](CC(=O)O)NC(=O)[C@H](CC(C)C)n3nc(CCN4CC(CF)C4)cc(C)c3=O)c(F)c3c2CCC3)c(C)c1] | 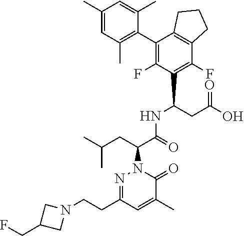 [SMILES: Cc1cc(C)c(-c2c(F)c([C@@H](CC(=O)O)NC(=O)[C@H](CC(C)C)n3nc(CCN4CC(CF)C4)cc(C)c3=O)c(F)c3c2CCC3)c(C)c1] |
| 385 | 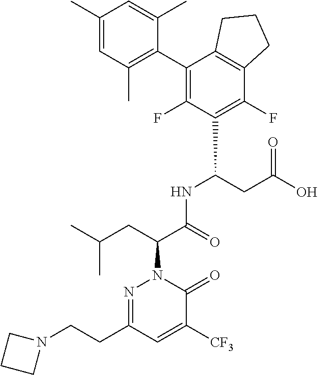 [SMILES: Cc1cc(C)c(-c2c(F)c([C@H](CC(=O)O)NC(=O)[C@H](CC(C)C)n3nc(CCN4CCC4)cc(C(F)(F)F)c3=O)c(F)c3c2CCC3)c(C)c1] | 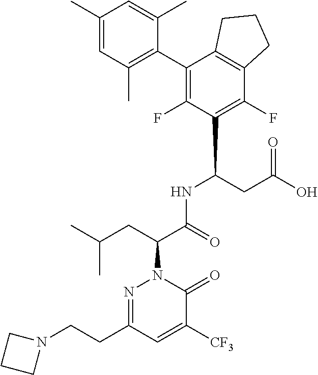 [SMILES: Cc1cc(C)c(-c2c(F)c([C@@H](CC(=O)O)NC(=O)[C@H](CC(C)C)n3nc(CCN4CCC4)cc(C(F)(F)F)c3=O)c(F)c3c2CCC3)c(C)c1] |
| 386 | 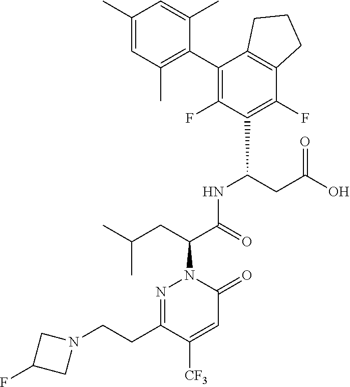 [SMILES: Cc1cc(C)c(-c2c(F)c([C@H](CC(=O)O)NC(=O)[C@H](CC(C)C)n3nc(CCN4CC(F)C4)c(C(F)(F)F)cc3=O)c(F)c3c2CCC3)c(C)c1] | 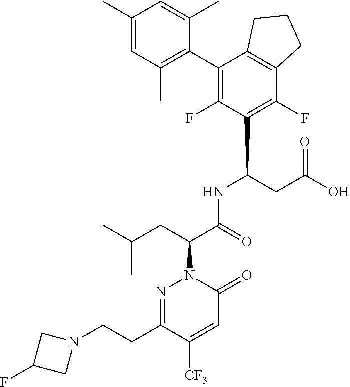 [SMILES: Cc1cc(C)c(-c2c(F)c([C@@H](CC(=O)O)NC(=O)[C@H](CC(C)C)n3nc(CCN4CC(F)C4)c(C(F)(F)F)cc3=O)c(F)c3c2CCC3)c(C)c1] |
| 387 | 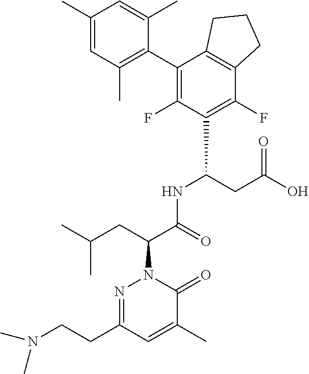 [SMILES: Cc1cc(C)c(-c2c(F)c([C@H](CC(=O)O)NC(=O)[C@H](CC(C)C)n3nc(CCN(C)C)cc(C)c3=O)c(F)c3c2CCC3)c(C)c1] | 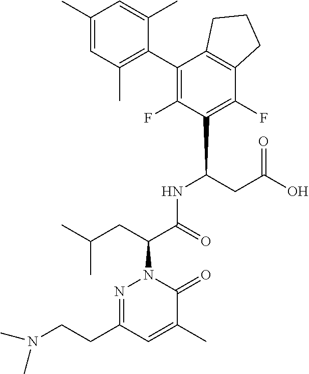 [SMILES: Cc1cc(C)c(-c2c(F)c([C@@H](CC(=O)O)NC(=O)[C@H](CC(C)C)n3nc(CCN(C)C)cc(C)c3=O)c(F)c3c2CCC3)c(C)c1] |
| 388 | 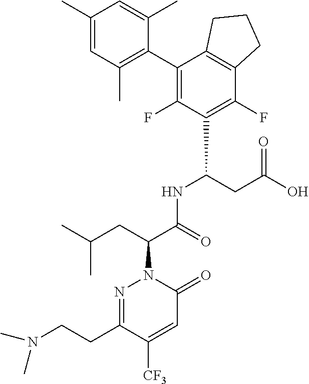 [SMILES: Cc1cc(C)c(-c2c(F)c([C@H](CC(=O)O)NC(=O)[C@H](CC(C)C)n3nc(CCN(C)C)c(C(F)(F)F)cc3=O)c(F)c3c2CCC3)c(C)c1] | 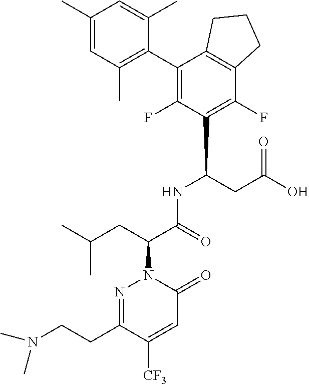 [SMILES: Cc1cc(C)c(-c2c(F)c([C@@H](CC(=O)O)NC(=O)[C@H](CC(C)C)n3nc(CCN(C)C)c(C(F)(F)F)cc3=O)c(F)c3c2CCC3)c(C)c1] |
| 389 | 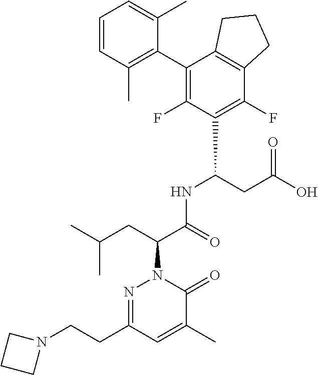 [SMILES: Cc1cccc(C)c1-c1c(F)c([C@H](CC(=O)O)NC(=O)[C@H](CC(C)C)n2nc(CCN3CCC3)cc(C)c2=O)c(F)c2c1CCC2] | 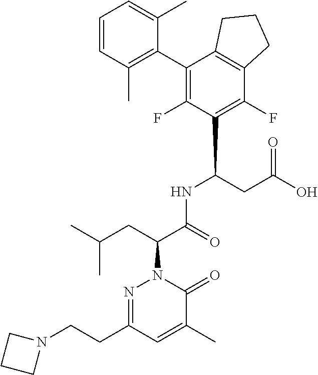 [SMILES: Cc1cccc(C)c1-c1c(F)c([C@@H](CC(=O)O)NC(=O)[C@H](CC(C)C)n2nc(CCN3CCC3)cc(C)c2=O)c(F)c2c1CCC2] |
| 390 | 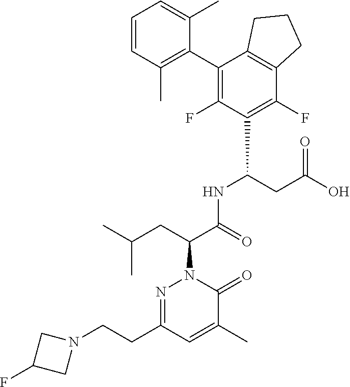 [SMILES: Cc1cccc(C)c1-c1c(F)c([C@H](CC(=O)O)NC(=O)[C@H](CC(C)C)n2nc(CCN3CC(F)C3)cc(C)c2=O)c(F)c2c1CCC2] | 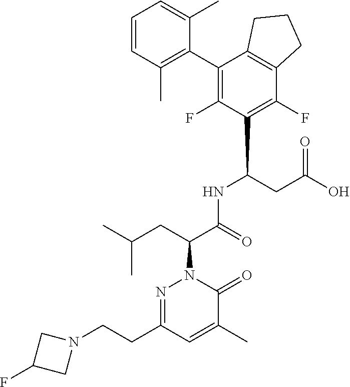 [SMILES: Cc1cccc(C)c1-c1c(F)c([C@@H](CC(=O)O)NC(=O)[C@H](CC(C)C)n2nc(CCN3CC(F)C3)cc(C)c2=O)c(F)c2c1CCC2] |
| 391 | 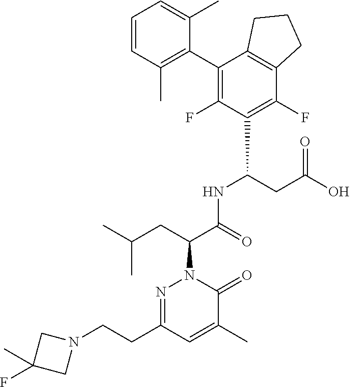 [SMILES: Cc1cccc(C)c1-c1c(F)c([C@H](CC(=O)O)NC(=O)[C@H](CC(C)C)n2nc(CCN3CC(C)(F)C3)cc(C)c2=O)c(F)c2c1CCC2] | 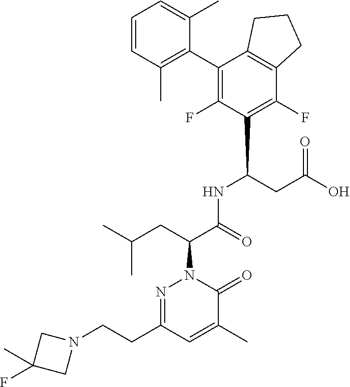 [SMILES: Cc1cccc(C)c1-c1c(F)c([C@@H](CC(=O)O)NC(=O)[C@H](CC(C)C)n2nc(CCN3CC(C)(F)C3)cc(C)c2=O)c(F)c2c1CCC2] |
| 392 | 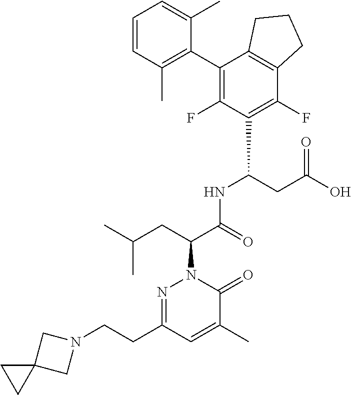 [SMILES: Cc1cccc(C)c1-c1c(F)c([C@H](CC(=O)O)NC(=O)[C@H](CC(C)C)n2nc(CCN3CC4(CC4)C3)cc(C)c2=O)c(F)c2c1CCC2] | 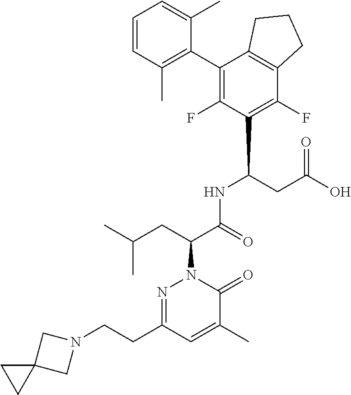 [SMILES: Cc1cccc(C)c1-c1c(F)c([C@@H](CC(=O)O)NC(=O)[C@H](CC(C)C)n2nc(CCN3CC4(CC4)C3)cc(C)c2=O)c(F)c2c1CCC2] |
| 393 | 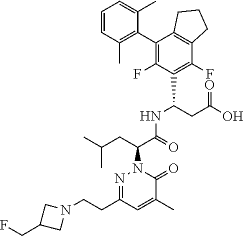 [SMILES: Cc1cccc(C)c1-c1c(F)c([C@H](CC(=O)O)NC(=O)[C@H](CC(C)C)n2nc(CCN3CC(CF)C3)cc(C)c2=O)c(F)c2c1CCC2] | 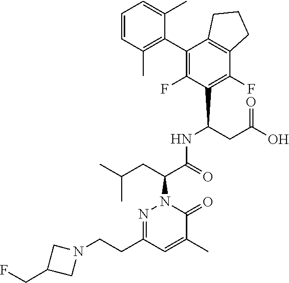 [SMILES: Cc1cccc(C)c1-c1c(F)c([C@@H](CC(=O)O)NC(=O)[C@H](CC(C)C)n2nc(CCN3CC(CF)C3)cc(C)c2=O)c(F)c2c1CCC2] |
| 394 | 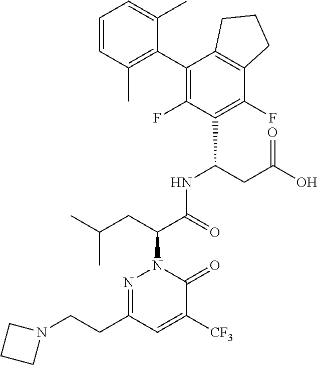 [SMILES: Cc1cccc(C)c1-c1c(F)c([C@H](CC(=O)O)NC(=O)[C@H](CC(C)C)n2nc(CCN3CCC3)cc(C(F)(F)F)c2=O)c(F)c2c1CCC2] | 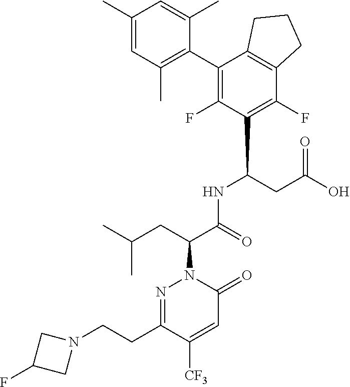 [SMILES: Cc1cc(C)c(-c2c(F)c([C@@H](CC(=O)O)NC(=O)[C@H](CC(C)C)n3nc(CCN4CC(F)C4)c(C(F)(F)F)cc3=O)c(F)c3c2CCC3)c(C)c1] |
| 395 | 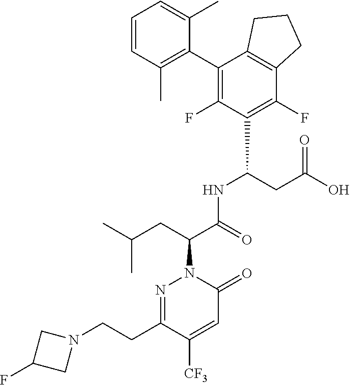 [SMILES: Cc1cccc(C)c1-c1c(F)c([C@H](CC(=O)O)NC(=O)[C@H](CC(C)C)n2nc(CCN3CC(F)C3)c(C(F)(F)F)cc2=O)c(F)c2c1CCC2] | 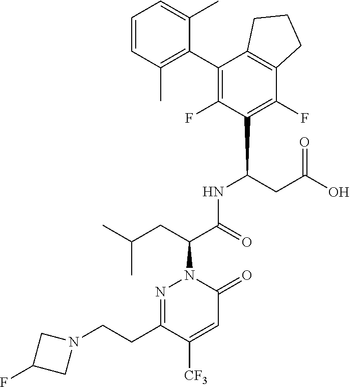 [SMILES: Cc1cccc(C)c1-c1c(F)c([C@@H](CC(=O)O)NC(=O)[C@H](CC(C)C)n2nc(CCN3CC(F)C3)c(C(F)(F)F)cc2=O)c(F)c2c1CCC2] |
| 396 | 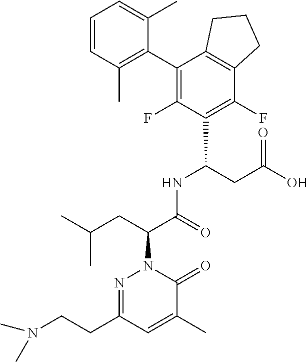 [SMILES: Cc1cccc(C)c1-c1c(F)c([C@H](CC(=O)O)NC(=O)[C@H](CC(C)C)n2nc(CCN(C)C)cc(C)c2=O)c(F)c2c1CCC2] | 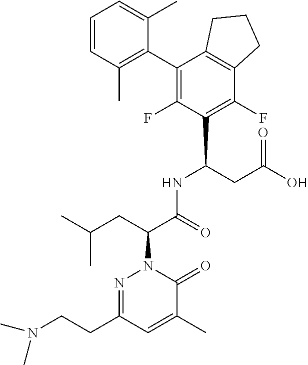 [SMILES: Cc1cccc(C)c1-c1c(F)c([C@@H](CC(=O)O)NC(=O)[C@H](CC(C)C)n2nc(CCN(C)C)cc(C)c2=O)c(F)c2c1CCC2] |
| 397 | 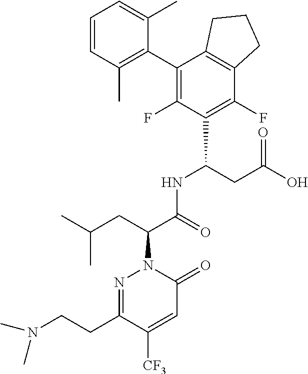 [SMILES: Cc1cccc(C)c1-c1c(F)c([C@H](CC(=O)O)NC(=O)[C@H](CC(C)C)n2nc(CCN(C)C)c(C(F)(F)F)cc2=O)c(F)c2c1CCC2] | 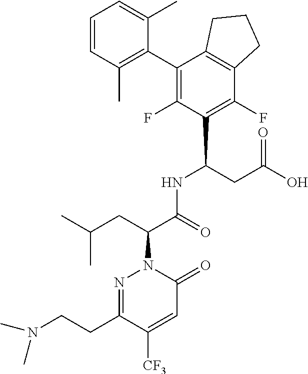 [SMILES: Cc1cccc(C)c1-c1c(F)c([C@@H](CC(=O)O)NC(=O)[C@H](CC(C)C)n2nc(CCN(C)C)c(C(F)(F)F)cc2=O)c(F)c2c1CCC2] |
| 398 | 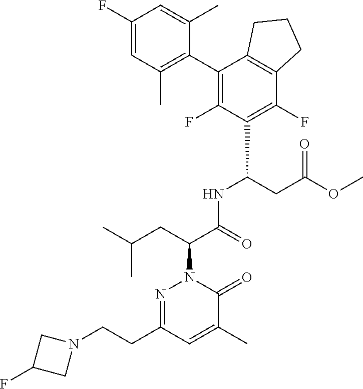 [SMILES: COC(=O)C[C@H](NC(=O)[C@H](CC(C)C)n1nc(CCN2CC(F)C2)cc(C)c1=O)c1c(F)c2c(c(-c3c(C)cc(F)cc3C)c1F)CCC2] | 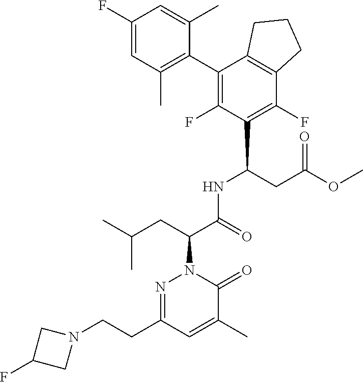 [SMILES: COC(=O)C[C@@H](NC(=O)[C@H](CC(C)C)n1nc(CCN2CC(F)C2)cc(C)c1=O)c1c(F)c2c(c(-c3c(C)cc(F)cc3C)c1F)CCC2] |
| 399 | 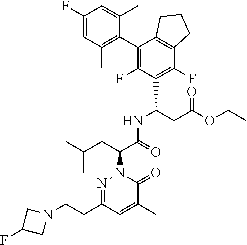 [SMILES: CCOC(=O)C[C@H](NC(=O)[C@H](CC(C)C)n1nc(CCN2CC(F)C2)cc(C)c1=O)c1c(F)c2c(c(-c3c(C)cc(F)cc3C)c1F)CCC2] | 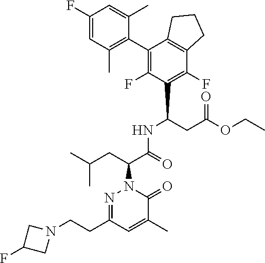 [SMILES: CCOC(=O)C[C@@H](NC(=O)[C@H](CC(C)C)n1nc(CCN2CC(F)C2)cc(C)c1=O)c1c(F)c2c(c(-c3c(C)cc(F)cc3C)c1F)CCC2] |
| 400 | 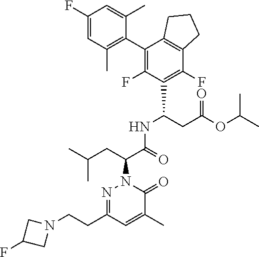 [SMILES: Cc1cc(F)cc(C)c1-c1c(F)c([C@H](CC(=O)OC(C)C)NC(=O)[C@H](CC(C)C)n2nc(CCN3CC(F)C3)cc(C)c2=O)c(F)c2c1CCC2] | 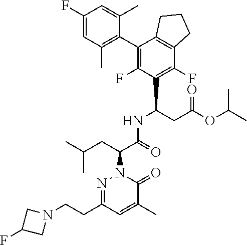 [SMILES: Cc1cc(F)cc(C)c1-c1c(F)c([C@@H](CC(=O)OC(C)C)NC(=O)[C@H](CC(C)C)n2nc(CCN3CC(F)C3)cc(C)c2=O)c(F)c2c1CCC2] |
| 401 | 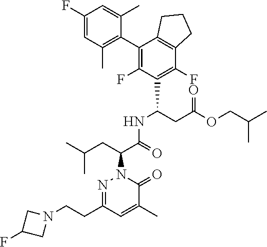 [SMILES: Cc1cc(F)cc(C)c1-c1c(F)c([C@H](CC(=O)OCC(C)C)NC(=O)[C@H](CC(C)C)n2nc(CCN3CC(F)C3)cc(C)c2=O)c(F)c2c1CCC2] | 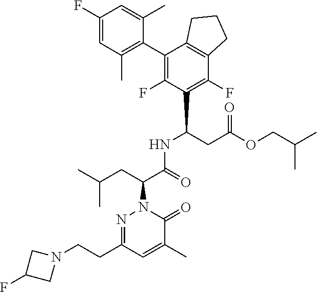 [SMILES: Cc1cc(F)cc(C)c1-c1c(F)c([C@@H](CC(=O)OCC(C)C)NC(=O)[C@H](CC(C)C)n2nc(CCN3CC(F)C3)cc(C)c2=O)c(F)c2c1CCC2] |
| 402 | 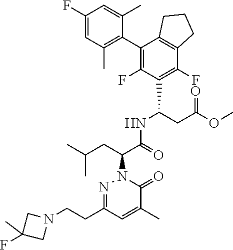 [SMILES: COC(=O)C[C@H](NC(=O)[C@H](CC(C)C)n1nc(CCN2CC(C)(F)C2)cc(C)c1=O)c1c(F)c2c(c(-c3c(C)cc(F)cc3C)c1F)CCC2] | 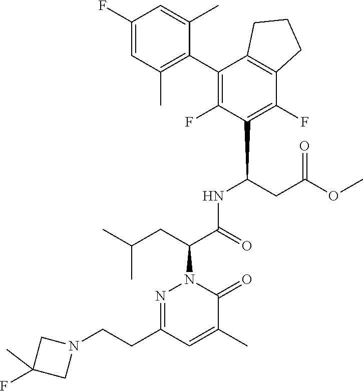 [SMILES: COC(=O)C[C@@H](NC(=O)[C@H](CC(C)C)n1nc(CCN2CC(C)(F)C2)cc(C)c1=O)c1c(F)c2c(c(-c3c(C)cc(F)cc3C)c1F)CCC2] |
| 403 | 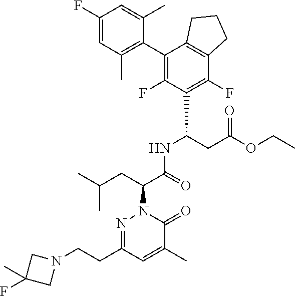 [SMILES: CCOC(=O)C[C@H](NC(=O)[C@H](CC(C)C)n1nc(CCN2CC(C)(F)C2)cc(C)c1=O)c1c(F)c2c(c(-c3c(C)cc(F)cc3C)c1F)CCC2] | 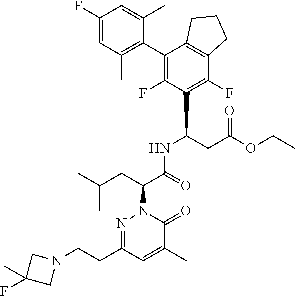 [SMILES: CCOC(=O)C[C@@H](NC(=O)[C@H](CC(C)C)n1nc(CCN2CC(C)(F)C2)cc(C)c1=O)c1c(F)c2c(c(-c3c(C)cc(F)cc3C)c1F)CCC2] |
| 404 | 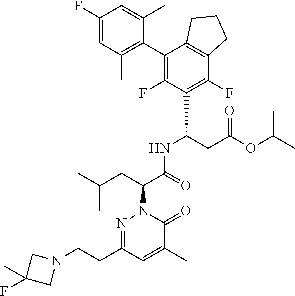 [SMILES: Cc1cc(F)cc(C)c1-c1c(F)c([C@H](CC(=O)OC(C)C)NC(=O)[C@H](CC(C)C)n2nc(CCN3CC(C)(F)C3)cc(C)c2=O)c(F)c2c1CCC2] | 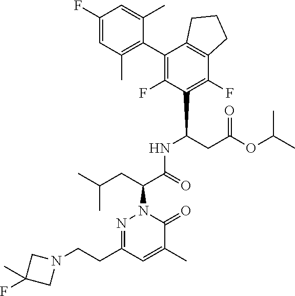 [SMILES: Cc1cc(F)cc(C)c1-c1c(F)c([C@@H](CC(=O)OC(C)C)NC(=O)[C@H](CC(C)C)n2nc(CCN3CC(C)(F)C3)cc(C)c2=O)c(F)c2c1CCC2] |
| 405 | 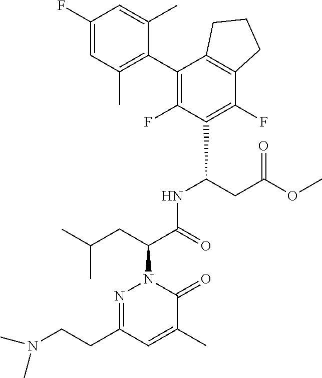 | 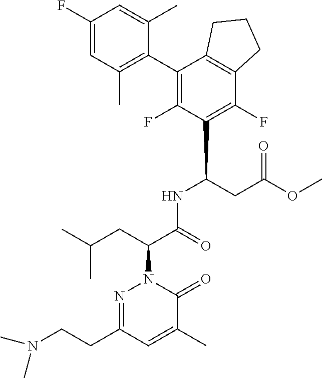 |
| 406 | 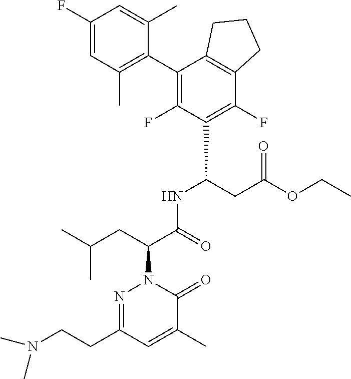 [SMILES: CCOC(=O)C[C@H](NC(=O)[C@H](CC(C)C)n1nc(CCN(C)C)cc(C)c1=O)c1c(F)c2c(c(-c3c(C)cc(F)cc3C)c1F)CCC2] | 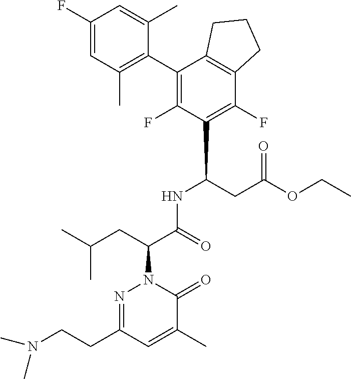 [SMILES: CCOC(=O)C[C@@H](NC(=O)[C@H](CC(C)C)n1nc(CCN(C)C)cc(C)c1=O)c1c(F)c2c(c(-c3c(C)cc(F)cc3C)c1F)CCC2] |
| 407 | 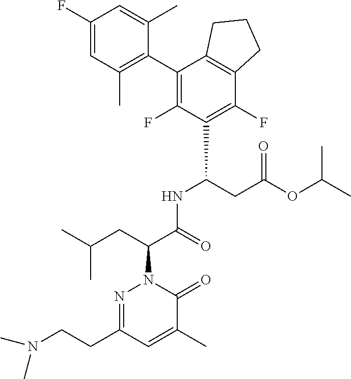 [SMILES: Cc1cc(F)cc(C)c1-c1c(F)c([C@H](CC(=O)OC(C)C)NC(=O)[C@H](CC(C)C)n2nc(CCN(C)C)cc(C)c2=O)c(F)c2c1CCC2] | 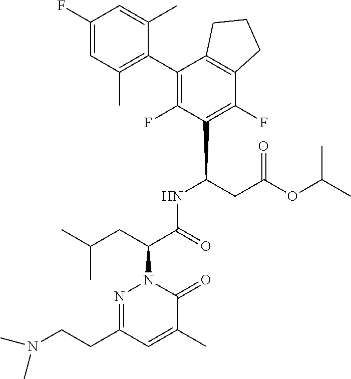 [SMILES: Cc1cc(F)cc(C)c1-c1c(F)c([C@@H](CC(=O)OC(C)C)NC(=O)[C@H](CC(C)C)n2nc(CCN(C)C)cc(C)c2=O)c(F)c2c1CCC2] |
| 408 | 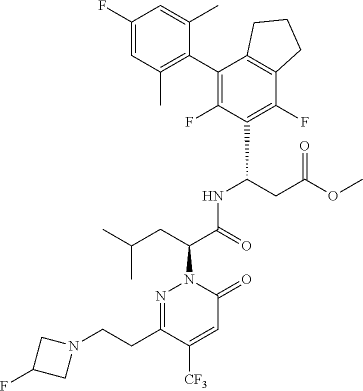 [SMILES: COC(=O)C[C@H](NC(=O)[C@H](CC(C)C)n1nc(CCN2CC(F)C2)c(C(F)(F)F)cc1=O)c1c(F)c2c(c(-c3c(C)cc(F)cc3C)c1F)CCC2] | 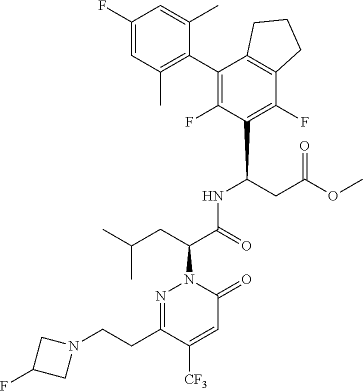 [SMILES: COC(=O)C[C@@H](NC(=O)[C@H](CC(C)C)n1nc(CCN2CC(F)C2)c(C(F)(F)F)cc1=O)c1c(F)c2c(c(-c3c(C)cc(F)cc3C)c1F)CCC2] |
| 409 | 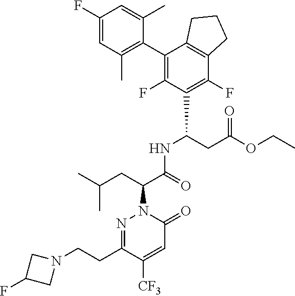 [SMILES: CCOC(=O)C[C@H](NC(=O)[C@H](CC(C)C)n1nc(CCN2CC(F)C2)c(C(F)(F)F)cc1=O)c1c(F)c2c(c(-c3c(C)cc(F)cc3C)c1F)CCC2] | 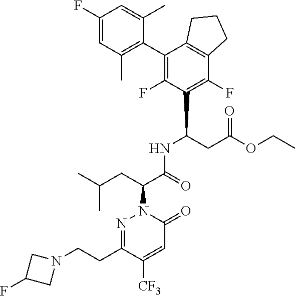 [SMILES: CCOC(=O)C[C@@H](NC(=O)[C@H](CC(C)C)n1nc(CCN2CC(F)C2)c(C(F)(F)F)cc1=O)c1c(F)c2c(c(-c3c(C)cc(F)cc3C)c1F)CCC2] |
| 410 | 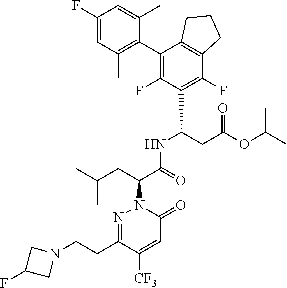 [SMILES: Cc1cc(F)cc(C)c1-c1c(F)c([C@H](CC(=O)OC(C)C)NC(=O)[C@H](CC(C)C)n2nc(CCN3CC(F)C3)c(C(F)(F)F)cc2=O)c(F)c2c1CCC2] | 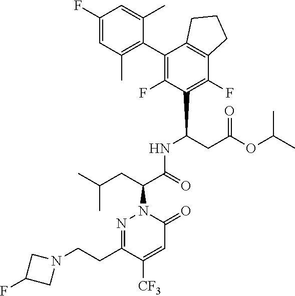 [SMILES: Cc1cc(F)cc(C)c1-c1c(F)c([C@@H](CC(=O)OC(C)C)NC(=O)[C@H](CC(C)C)n2nc(CCN3CC(F)C3)c(C(F)(F)F)cc2=O)c(F)c2c1CCC2] |
| 411 | 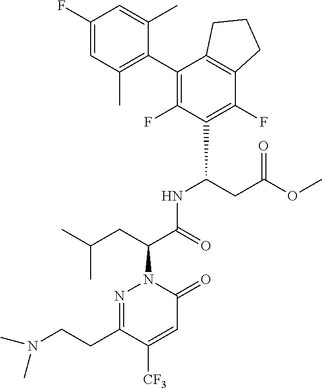 | 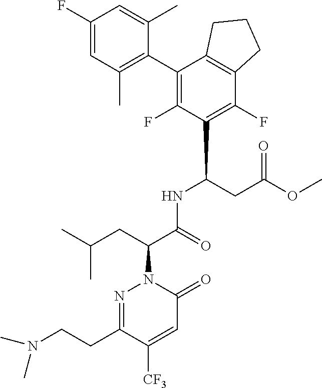 |
| 412 | 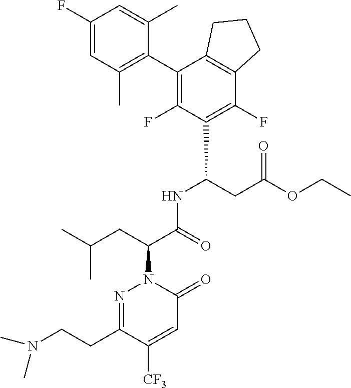 [SMILES: CCOC(=O)C[C@H](NC(=O)[C@H](CC(C)C)n1nc(CCN(C)C)c(C(F)(F)F)cc1=O)c1c(F)c2c(c(-c3c(C)cc(F)cc3C)c1F)CCC2] | 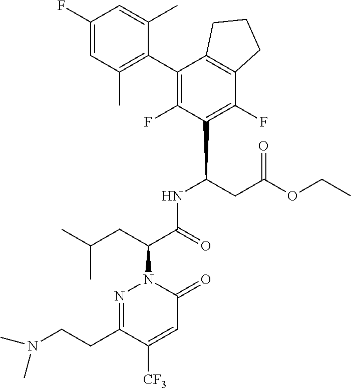 [SMILES: CCOC(=O)C[C@@H](NC(=O)[C@H](CC(C)C)n1nc(CCN(C)C)c(C(F)(F)F)cc1=O)c1c(F)c2c(c(-c3c(C)cc(F)cc3C)c1F)CCC2] |
| 413 | 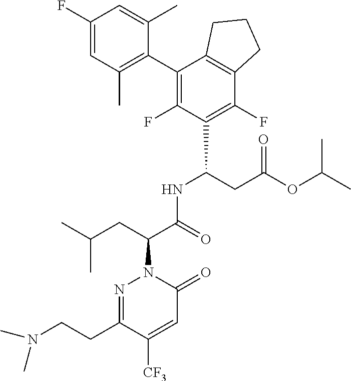 [SMILES: Cc1cc(F)cc(C)c1-c1c(F)c([C@H](CC(=O)OC(C)C)NC(=O)[C@H](CC(C)C)n2nc(CCN(C)C)c(C(F)(F)F)cc2=O)c(F)c2c1CCC2] | 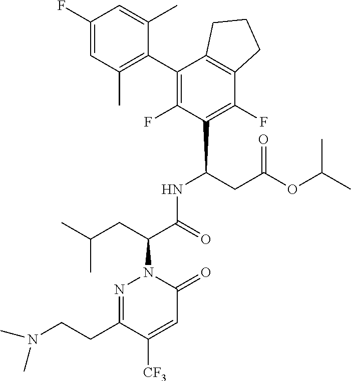 [SMILES: Cc1cc(F)cc(C)c1-c1c(F)c([C@@H](CC(=O)OC(C)C)NC(=O)[C@H](CC(C)C)n2nc(CCN(C)C)c(C(F)(F)F)cc2=O)c(F)c2c1CCC2] |
| 414 | 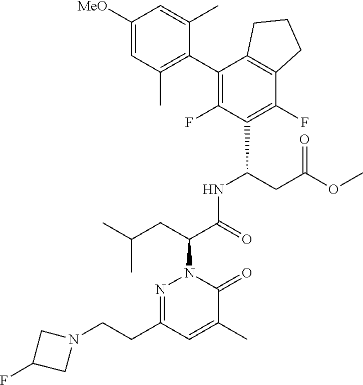 [SMILES: COC(=O)C[C@H](NC(=O)[C@H](CC(C)C)n1nc(CCN2CC(F)C2)cc(C)c1=O)c1c(F)c2c(c(-c3c(C)cc(OC)cc3C)c1F)CCC2] | 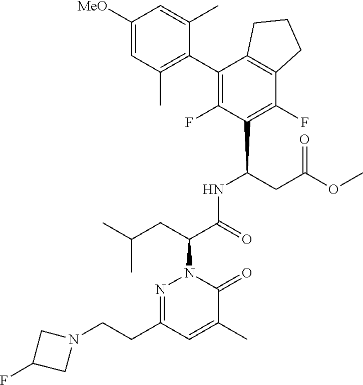 [SMILES: COC(=O)C[C@@H](NC(=O)[C@H](CC(C)C)n1nc(CCN2CC(F)C2)cc(C)c1=O)c1c(F)c2c(c(-c3c(C)cc(OC)cc3C)c1F)CCC2] |
| 415 | 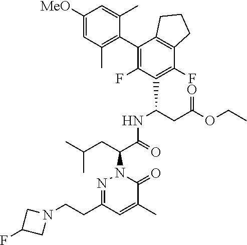 [SMILES: CCOC(=O)C[C@H](NC(=O)[C@H](CC(C)C)n1nc(CCN2CC(F)C2)cc(C)c1=O)c1c(F)c2c(c(-c3c(C)cc(OC)cc3C)c1F)CCC2] | 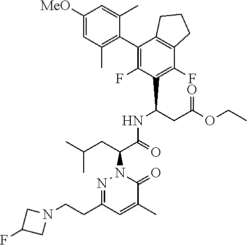 [SMILES: CCOC(=O)C[C@@H](NC(=O)[C@H](CC(C)C)n1nc(CCN2CC(F)C2)cc(C)c1=O)c1c(F)c2c(c(-c3c(C)cc(OC)cc3C)c1F)CCC2] |
| 416 | 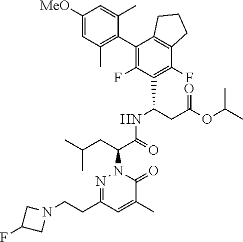 [SMILES: COc1cc(C)c(-c2c(F)c([C@H](CC(=O)OC(C)C)NC(=O)[C@H](CC(C)C)n3nc(CCN4CC(F)C4)cc(C)c3=O)c(F)c3c2CCC3)c(C)c1] | 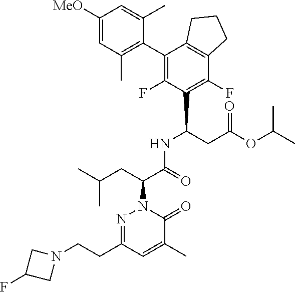 [SMILES: COc1cc(C)c(-c2c(F)c([C@@H](CC(=O)OC(C)C)NC(=O)[C@H](CC(C)C)n3nc(CCN4CC(F)C4)cc(C)c3=O)c(F)c3c2CCC3)c(C)c1] |
| 417 | 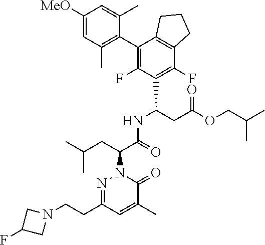 [SMILES: COc1cc(C)c(-c2c(F)c([C@H](CC(=O)OCC(C)C)NC(=O)[C@H](CC(C)C)n3nc(CCN4CC(F)C4)cc(C)c3=O)c(F)c3c2CCC3)c(C)c1] | 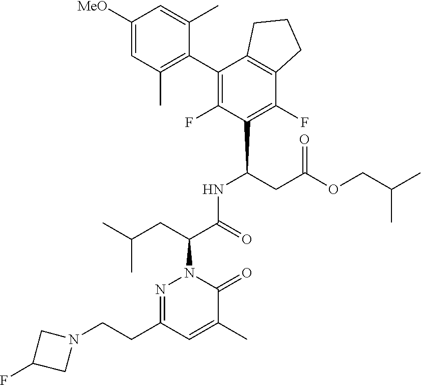 [SMILES: COc1cc(C)c(-c2c(F)c([C@@H](CC(=O)OCC(C)C)NC(=O)[C@H](CC(C)C)n3nc(CCN4CC(F)C4)cc(C)c3=O)c(F)c3c2CCC3)c(C)c1] |
| 418 | 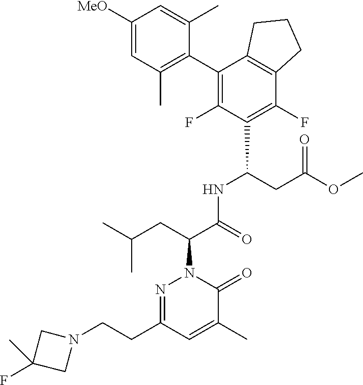 [SMILES: COC(=O)C[C@H](NC(=O)[C@H](CC(C)C)n1nc(CCN2CC(C)(F)C2)cc(C)c1=O)c1c(F)c2c(c(-c3c(C)cc(OC)cc3C)c1F)CCC2] | 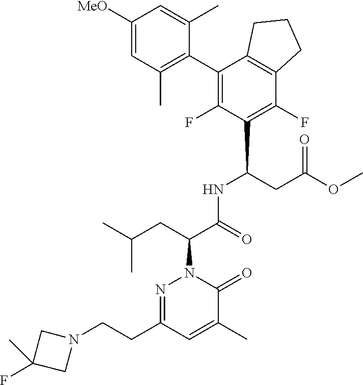 [SMILES: COC(=O)C[C@@H](NC(=O)[C@H](CC(C)C)n1nc(CCN2CC(C)(F)C2)cc(C)c1=O)c1c(F)c2c(c(-c3c(C)cc(OC)cc3C)c1F)CCC2] |
| 419 | 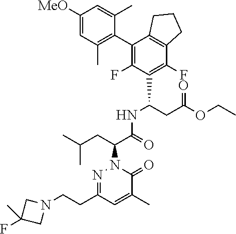 [SMILES: CCOC(=O)C[C@H](NC(=O)[C@H](CC(C)C)n1nc(CCN2CC(C)(F)C2)cc(C)c1=O)c1c(F)c2c(c(-c3c(C)cc(OC)cc3C)c1F)CCC2] | 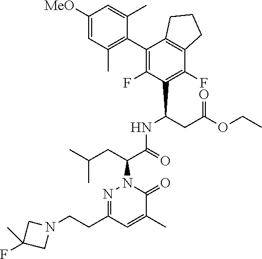 [SMILES: CCOC(=O)C[C@@H](NC(=O)[C@H](CC(C)C)n1nc(CCN2CC(C)(F)C2)cc(C)c1=O)c1c(F)c2c(c(-c3c(C)cc(OC)cc3C)c1F)CCC2] |
| 420 | 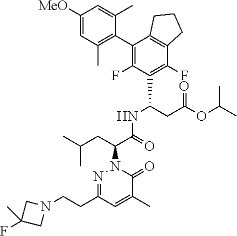 [SMILES: COc1cc(C)c(-c2c(F)c([C@H](CC(=O)OC(C)C)NC(=O)[C@H](CC(C)C)n3nc(CCN4CC(C)(F)C4)cc(C)c3=O)c(F)c3c2CCC3)c(C)c1] | 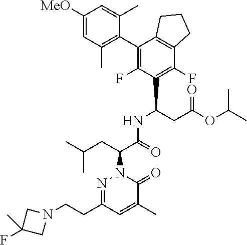 [SMILES: COc1cc(C)c(-c2c(F)c([C@@H](CC(=O)OC(C)C)NC(=O)[C@H](CC(C)C)n3nc(CCN4CC(C)(F)C4)cc(C)c3=O)c(F)c3c2CCC3)c(C)c1] |
| 421 | 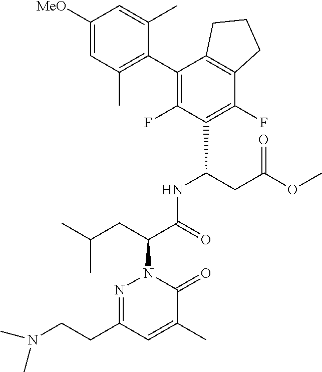 | 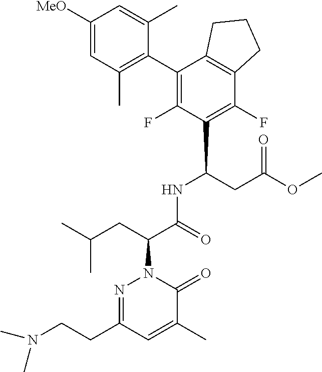 |
| 422 | 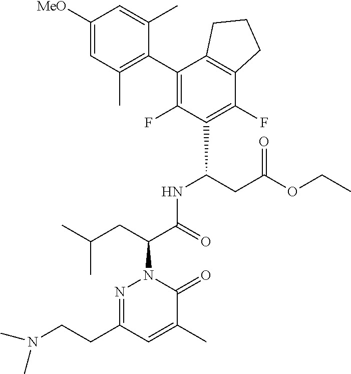 [SMILES: CCOC(=O)C[C@H](NC(=O)[C@H](CC(C)C)n1nc(CCN(C)C)cc(C)c1=O)c1c(F)c2c(c(-c3c(C)cc(OC)cc3C)c1F)CCC2] | 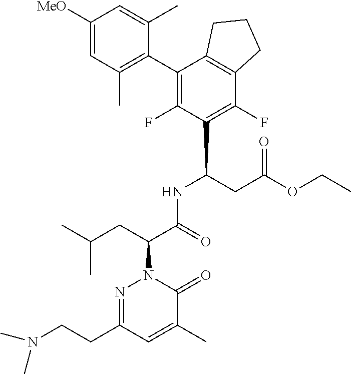 [SMILES: CCOC(=O)C[C@@H](NC(=O)[C@H](CC(C)C)n1nc(CCN(C)C)cc(C)c1=O)c1c(F)c2c(c(-c3c(C)cc(OC)cc3C)c1F)CCC2] |
| 423 | 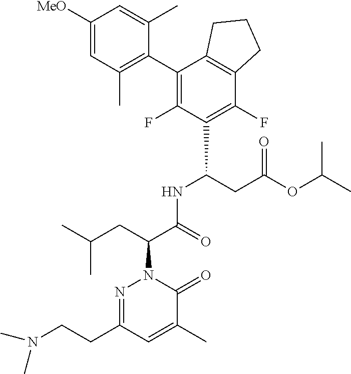 [SMILES: COc1cc(C)c(-c2c(F)c([C@H](CC(=O)OC(C)C)NC(=O)[C@H](CC(C)C)n3nc(CCN(C)C)cc(C)c3=O)c(F)c3c2CCC3)c(C)c1] | 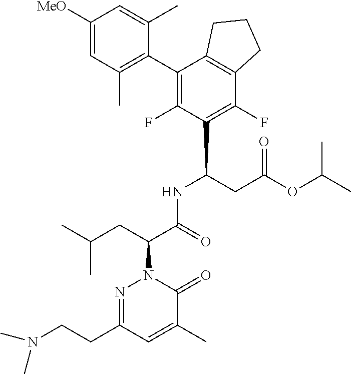 [SMILES: COc1cc(C)c(-c2c(F)c([C@@H](CC(=O)OC(C)C)NC(=O)[C@H](CC(C)C)n3nc(CCN(C)C)cc(C)c3=O)c(F)c3c2CCC3)c(C)c1] |
| 424 | 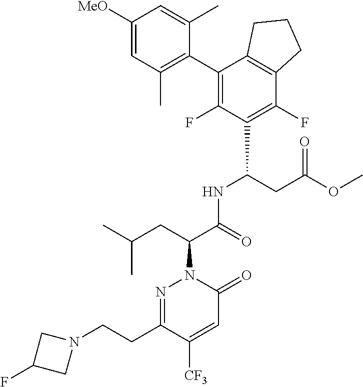 [SMILES: COC(=O)C[C@H](NC(=O)[C@H](CC(C)C)n1nc(CCN2CC(F)C2)c(C(F)(F)F)cc1=O)c1c(F)c2c(c(-c3c(C)cc(OC)cc3C)c1F)CCC2] | 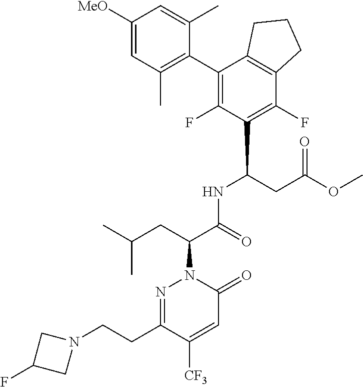 [SMILES: COC(=O)C[C@@H](NC(=O)[C@H](CC(C)C)n1nc(CCN2CC(F)C2)c(C(F)(F)F)cc1=O)c1c(F)c2c(c(-c3c(C)cc(OC)cc3C)c1F)CCC2] |
| 425 | 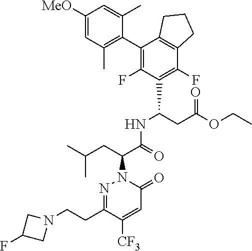 [SMILES: CCOC(=O)C[C@H](NC(=O)[C@H](CC(C)C)n1nc(CCN2CC(F)C2)c(C(F)(F)F)cc1=O)c1c(F)c2c(c(-c3c(C)cc(OC)cc3C)c1F)CCC2] | 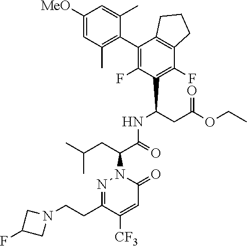 [SMILES: CCOC(=O)C[C@@H](NC(=O)[C@H](CC(C)C)n1nc(CCN2CC(F)C2)c(C(F)(F)F)cc1=O)c1c(F)c2c(c(-c3c(C)cc(OC)cc3C)c1F)CCC2] |
| 426 | 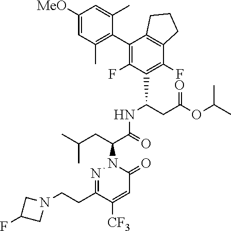 [SMILES: COc1cc(C)c(-c2c(F)c([C@H](CC(=O)OC(C)C)NC(=O)[C@H](CC(C)C)n3nc(CCN4CC(F)C4)c(C(F)(F)F)cc3=O)c(F)c3c2CCC3)c(C)c1] | 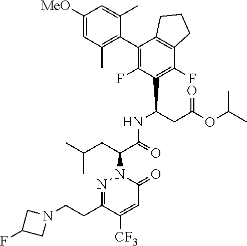 [SMILES: COc1cc(C)c(-c2c(F)c([C@@H](CC(=O)OC(C)C)NC(=O)[C@H](CC(C)C)n3nc(CCN4CC(F)C4)c(C(F)(F)F)cc3=O)c(F)c3c2CCC3)c(C)c1] |
| 427 | 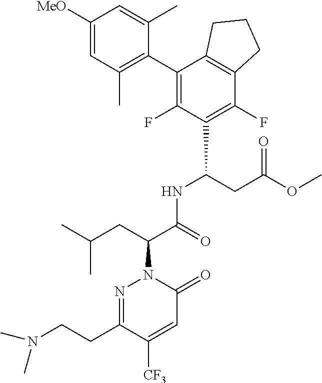 | 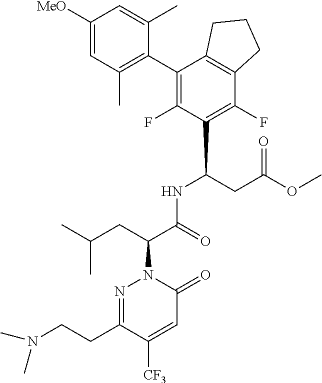 |
| 428 | 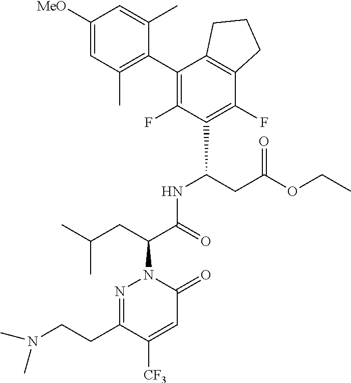 [SMILES: CCOC(=O)C[C@H](NC(=O)[C@H](CC(C)C)n1nc(CCN(C)C)c(C(F)(F)F)cc1=O)c1c(F)c2c(c(-c3c(C)cc(OC)cc3C)c1F)CCC2] | 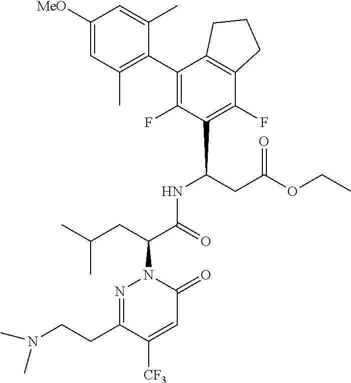 [SMILES: CCOC(=O)C[C@@H](NC(=O)[C@H](CC(C)C)n1nc(CCN(C)C)c(C(F)(F)F)cc1=O)c1c(F)c2c(c(-c3c(C)cc(OC)cc3C)c1F)CCC2] |
| 429 | 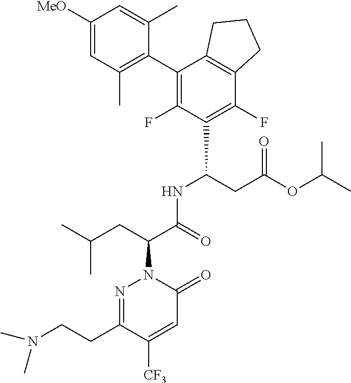 [SMILES: COc1cc(C)c(-c2c(F)c([C@H](CC(=O)OC(C)C)NC(=O)[C@H](CC(C)C)n3nc(CCN(C)C)c(C(F)(F)F)cc3=O)c(F)c3c2CCC3)c(C)c1] | 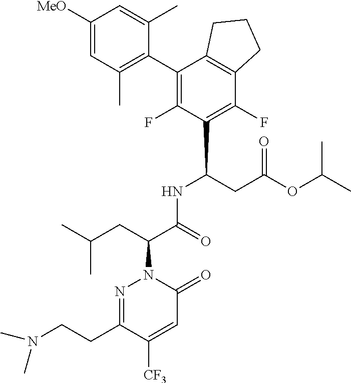 [SMILES: COc1cc(C)c(-c2c(F)c([C@@H](CC(=O)OC(C)C)NC(=O)[C@H](CC(C)C)n3nc(CCN(C)C)c(C(F)(F)F)cc3=O)c(F)c3c2CCC3)c(C)c1] |
| 430 | 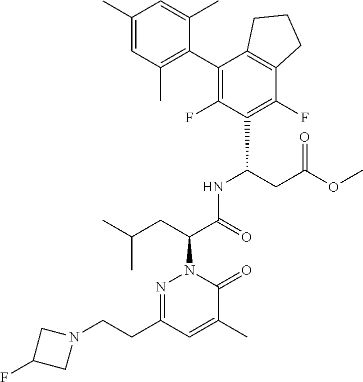 [SMILES: COC(=O)C[C@H](NC(=O)[C@H](CC(C)C)n1nc(CCN2CC(F)C2)cc(C)c1=O)c1c(F)c2c(c(-c3c(C)cc(C)cc3C)c1F)CCC2] | 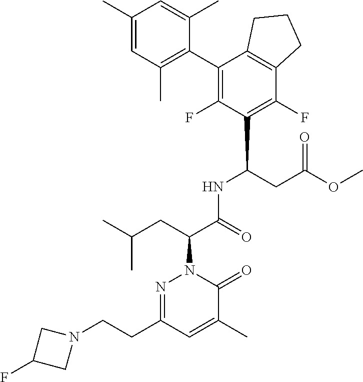 [SMILES: COC(=O)C[C@@H](NC(=O)[C@H](CC(C)C)n1nc(CCN2CC(F)C2)cc(C)c1=O)c1c(F)c2c(c(-c3c(C)cc(C)cc3C)c1F)CCC2] |
| 431 | 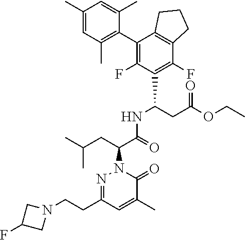 [SMILES: CCOC(=O)C[C@H](NC(=O)[C@H](CC(C)C)n1nc(CCN2CC(F)C2)cc(C)c1=O)c1c(F)c2c(c(-c3c(C)cc(C)cc3C)c1F)CCC2] | 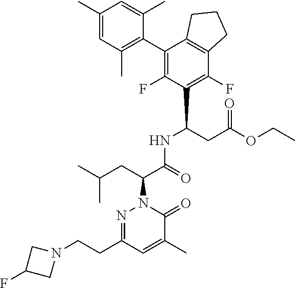 [SMILES: CCOC(=O)C[C@@H](NC(=O)[C@H](CC(C)C)n1nc(CCN2CC(F)C2)cc(C)c1=O)c1c(F)c2c(c(-c3c(C)cc(C)cc3C)c1F)CCC2] |
| 432 | 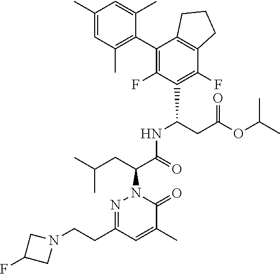 [SMILES: Cc1cc(C)c(-c2c(F)c([C@H](CC(=O)OC(C)C)NC(=O)[C@H](CC(C)C)n3nc(CCN4CC(F)C4)cc(C)c3=O)c(F)c3c2CCC3)c(C)c1] | 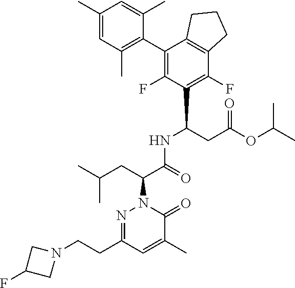 [SMILES: Cc1cc(C)c(-c2c(F)c([C@@H](CC(=O)OC(C)C)NC(=O)[C@H](CC(C)C)n3nc(CCN4CC(F)C4)cc(C)c3=O)c(F)c3c2CCC3)c(C)c1] |
| 433 | 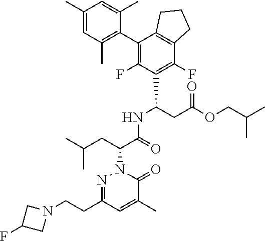 [SMILES: Cc1cc(C)c(-c2c(F)c([C@H](CC(=O)OCC(C)C)NC(=O)[C@@H](CC(C)C)n3nc(CCN4CC(F)C4)cc(C)c3=O)c(F)c3c2CCC3)c(C)c1] | 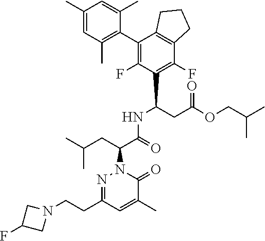 [SMILES: Cc1cc(C)c(-c2c(F)c([C@@H](CC(=O)OCC(C)C)NC(=O)[C@H](CC(C)C)n3nc(CCN4CC(F)C4)cc(C)c3=O)c(F)c3c2CCC3)c(C)c1] |
| 434 | 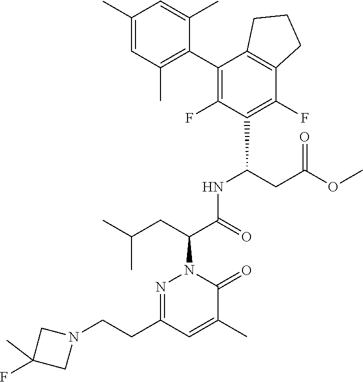 [SMILES: COC(=O)C[C@H](NC(=O)[C@H](CC(C)C)n1nc(CCN2CC(C)(F)C2)cc(C)c1=O)c1c(F)c2c(c(-c3c(C)cc(C)cc3C)c1F)CCC2] | 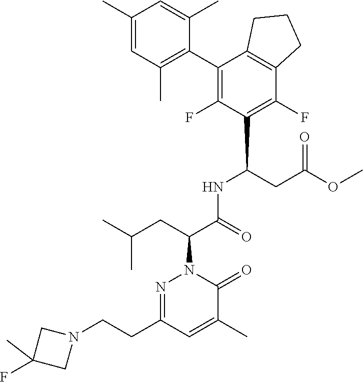 [SMILES: COC(=O)C[C@@H](NC(=O)[C@H](CC(C)C)n1nc(CCN2CC(C)(F)C2)cc(C)c1=O)c1c(F)c2c(c(-c3c(C)cc(C)cc3C)c1F)CCC2] |
| 435 | 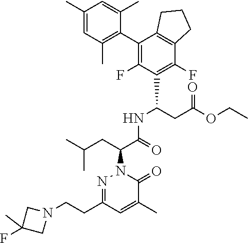 [SMILES: CCOC(=O)C[C@H](NC(=O)[C@H](CC(C)C)n1nc(CCN2CC(C)(F)C2)cc(C)c1=O)c1c(F)c2c(c(-c3c(C)cc(C)cc3C)c1F)CCC2] | 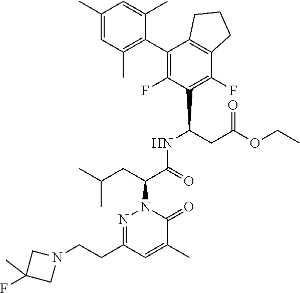 [SMILES: CCOC(=O)C[C@@H](NC(=O)[C@H](CC(C)C)n1nc(CCN2CC(C)(F)C2)cc(C)c1=O)c1c(F)c2c(c(-c3c(C)cc(C)cc3C)c1F)CCC2] |
| 436 | 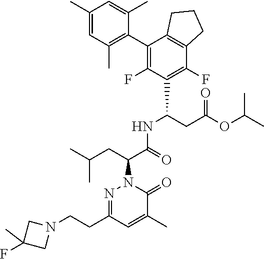 [SMILES: Cc1cc(C)c(-c2c(F)c([C@H](CC(=O)OC(C)C)NC(=O)[C@H](CC(C)C)n3nc(CCN4CC(C)(F)C4)cc(C)c3=O)c(F)c3c2CCC3)c(C)c1] | 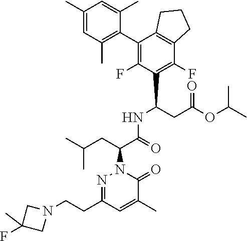 [SMILES: Cc1cc(C)c(-c2c(F)c([C@@H](CC(=O)OC(C)C)NC(=O)[C@H](CC(C)C)n3nc(CCN4CC(C)(F)C4)cc(C)c3=O)c(F)c3c2CCC3)c(C)c1] |
| 437 | 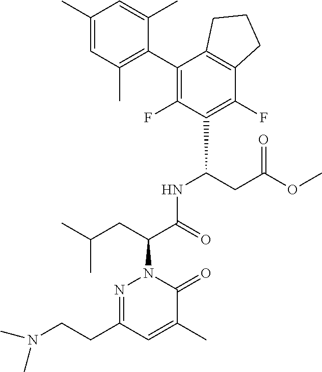 | 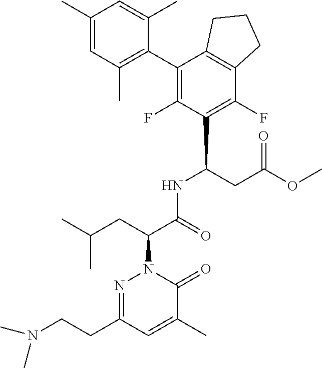 |
| 438 | 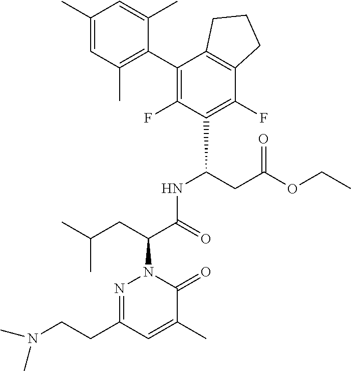 [SMILES: CCOC(=O)C[C@H](NC(=O)[C@H](CC(C)C)n1nc(CCN(C)C)cc(C)c1=O)c1c(F)c2c(c(-c3c(C)cc(C)cc3C)c1F)CCC2] | 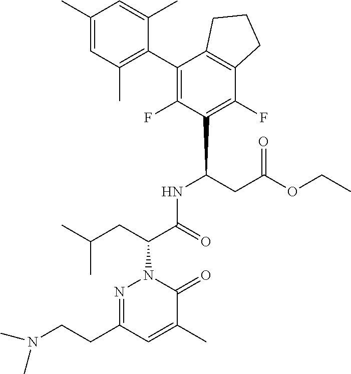 [SMILES: CCOC(=O)C[C@@H](NC(=O)[C@@H](CC(C)C)n1nc(CCN(C)C)cc(C)c1=O)c1c(F)c2c(c(-c3c(C)cc(C)cc3C)c1F)CCC2] |
| 439 | 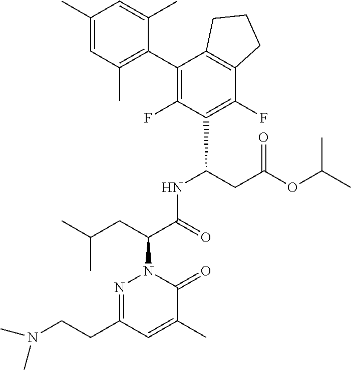 [SMILES: Cc1cc(C)c(-c2c(F)c([C@H](CC(=O)OC(C)C)NC(=O)[C@H](CC(C)C)n3nc(CCN(C)C)cc(C)c3=O)c(F)c3c2CCC3)c(C)c1] | 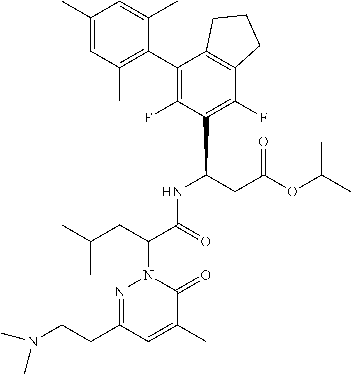 [SMILES: Cc1cc(C)c(-c2c(F)c([C@@H](CC(=O)OC(C)C)NC(=O)C(CC(C)C)n3nc(CCN(C)C)cc(C)c3=O)c(F)c3c2CCC3)c(C)c1] |
| 440 | 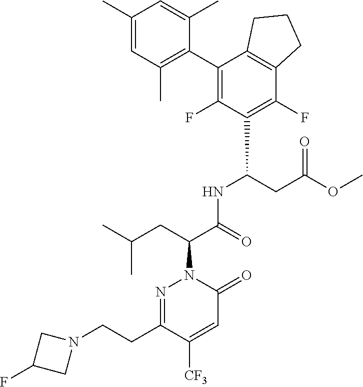 [SMILES: COC(=O)C[C@H](NC(=O)[C@H](CC(C)C)n1nc(CCN2CC(F)C2)c(C(F)(F)F)cc1=O)c1c(F)c2c(c(-c3c(C)cc(C)cc3C)c1F)CCC2] | 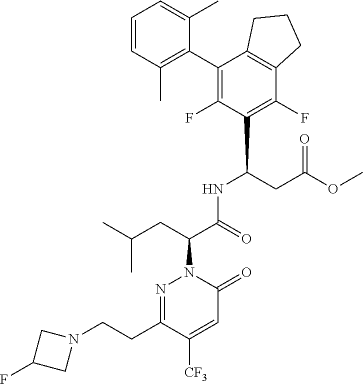 [SMILES: COC(=O)C[C@@H](NC(=O)[C@H](CC(C)C)n1nc(CCN2CC(F)C2)c(C(F)(F)F)cc1=O)c1c(F)c2c(c(-c3c(C)cccc3C)c1F)CCC2] |
| 441 | 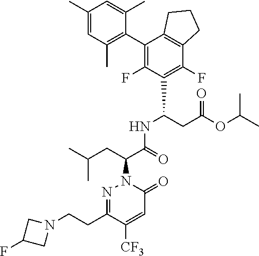 [SMILES: Cc1cc(C)c(-c2c(F)c([C@H](CC(=O)OC(C)C)NC(=O)[C@H](CC(C)C)n3nc(CCN4CC(F)C4)c(C(F)(F)F)cc3=O)c(F)c3c2CCC3)c(C)c1] | 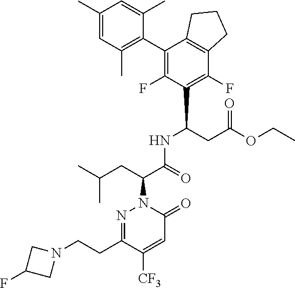 [SMILES: CCOC(=O)C[C@@H](NC(=O)[C@H](CC(C)C)n1nc(CCN2CC(F)C2)c(C(F)(F)F)cc1=O)c1c(F)c2c(c(-c3c(C)cc(C)cc3C)c1F)CCC2] |
| 442 | 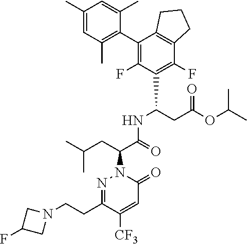 [SMILES: Cc1cc(C)c(-c2c(F)c([C@H](CC(=O)OC(C)C)NC(=O)[C@H](CC(C)C)n3nc(CCN4CC(F)C4)c(C(F)(F)F)cc3=O)c(F)c3c2CCC3)c(C)c1] | 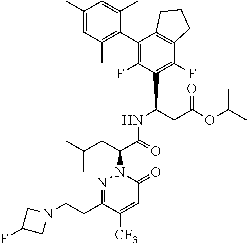 [SMILES: Cc1cc(C)c(-c2c(F)c([C@@H](CC(=O)OC(C)C)NC(=O)[C@H](CC(C)C)n3nc(CCN4CC(F)C4)c(C(F)(F)F)cc3=O)c(F)c3c2CCC3)c(C)c1] |
| 443 | 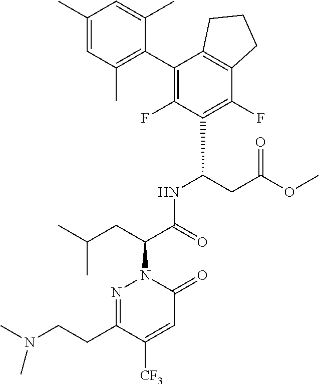 | 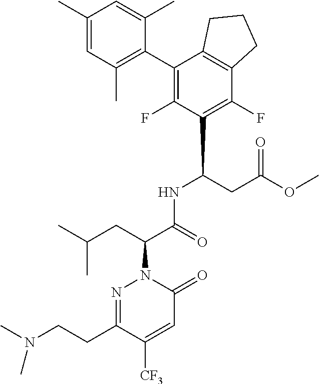 |
| 444 | 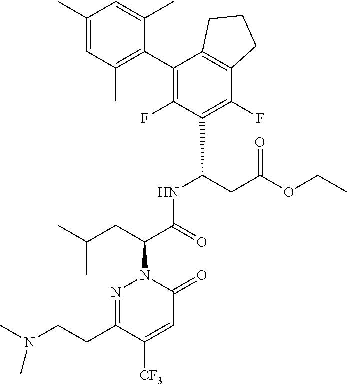 [SMILES: CCOC(=O)C[C@H](NC(=O)[C@H](CC(C)C)n1nc(CCN(C)C)c(C(F)(F)F)cc1=O)c1c(F)c2c(c(-c3c(C)cc(C)cc3C)c1F)CCC2] | 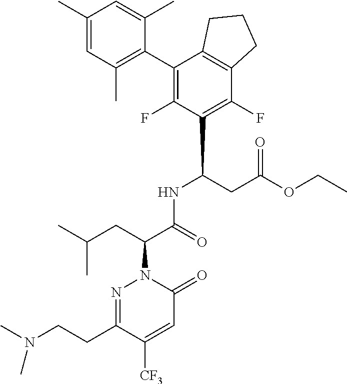 [SMILES: CCOC(=O)C[C@@H](NC(=O)[C@H](CC(C)C)n1nc(CCN(C)C)c(C(F)(F)F)cc1=O)c1c(F)c2c(c(-c3c(C)cc(C)cc3C)c1F)CCC2] |
| 445 | 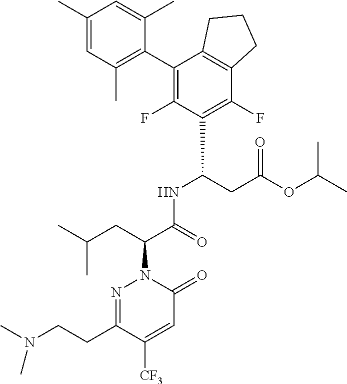 [SMILES: Cc1cc(C)c(-c2c(F)c([C@H](CC(=O)OC(C)C)NC(=O)[C@H](CC(C)C)n3nc(CCN(C)C)c(C(F)(F)F)cc3=O)c(F)c3c2CCC3)c(C)c1] | 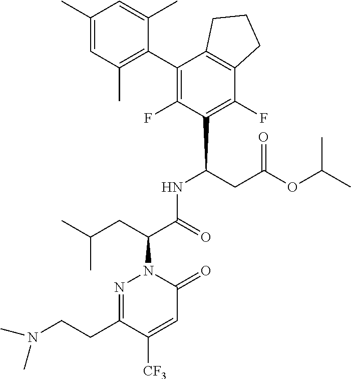 [SMILES: Cc1cc(C)c(-c2c(F)c([C@@H](CC(=O)OC(C)C)NC(=O)[C@H](CC(C)C)n3nc(CCN(C)C)c(C(F)(F)F)cc3=O)c(F)c3c2CCC3)c(C)c1] |
| 446 | 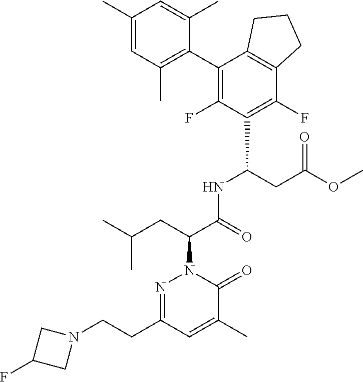 [SMILES: COC(=O)C[C@H](NC(=O)[C@H](CC(C)C)n1nc(CCN2CC(F)C2)cc(C)c1=O)c1c(F)c2c(c(-c3c(C)cc(C)cc3C)c1F)CCC2] | 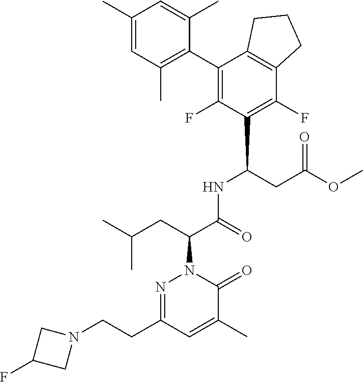 [SMILES: COC(=O)C[C@@H](NC(=O)[C@H](CC(C)C)n1nc(CCN2CC(F)C2)cc(C)c1=O)c1c(F)c2c(c(-c3c(C)cc(C)cc3C)c1F)CCC2] |
| 447 | 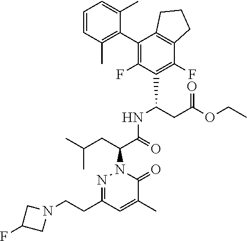 [SMILES: CCOC(=O)C[C@H](NC(=O)[C@H](CC(C)C)n1nc(CCN2CC(F)C2)cc(C)c1=O)c1c(F)c2c(c(-c3c(C)cccc3C)c1F)CCC2] | 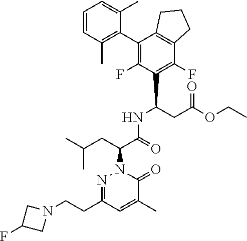 [SMILES: CCOC(=O)C[C@@H](NC(=O)[C@H](CC(C)C)n1nc(CCN2CC(F)C2)cc(C)c1=O)c1c(F)c2c(c(-c3c(C)cccc3C)c1F)CCC2] |
| 448 | 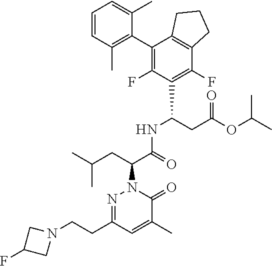 [SMILES: Cc1cccc(C)c1-c1c(F)c([C@H](CC(=O)OC(C)C)NC(=O)[C@H](CC(C)C)n2nc(CCN3CC(F)C3)cc(C)c2=O)c(F)c2c1CCC2] | 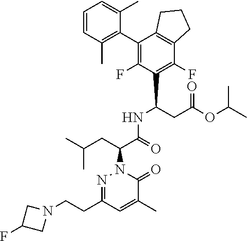 [SMILES: Cc1cccc(C)c1-c1c(F)c([C@@H](CC(=O)OC(C)C)NC(=O)[C@H](CC(C)C)n2nc(CCN3CC(F)C3)cc(C)c2=O)c(F)c2c1CCC2] |
| 449 | 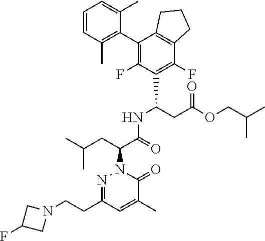 [SMILES: Cc1cccc(C)c1-c1c(F)c([C@H](CC(=O)OCC(C)C)NC(=O)[C@H](CC(C)C)n2nc(CCN3CC(F)C3)cc(C)c2=O)c(F)c2c1CCC2] | 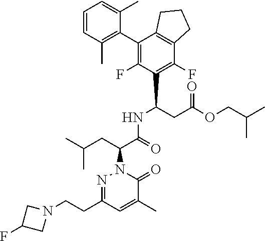 [SMILES: Cc1cccc(C)c1-c1c(F)c([C@@H](CC(=O)OCC(C)C)NC(=O)[C@H](CC(C)C)n2nc(CCN3CC(F)C3)cc(C)c2=O)c(F)c2c1CCC2] |
| 450 | 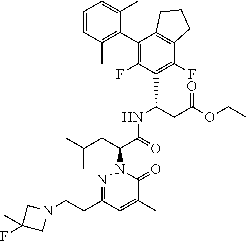 [SMILES: CCOC(=O)C[C@H](NC(=O)[C@H](CC(C)C)n1nc(CCN2CC(C)(F)C2)cc(C)c1=O)c1c(F)c2c(c(-c3c(C)cccc3C)c1F)CCC2] | 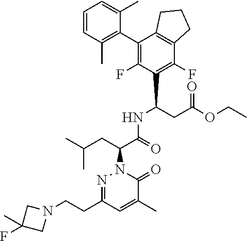 [SMILES: CCOC(=O)C[C@@H](NC(=O)[C@H](CC(C)C)n1nc(CCN2CC(C)(F)C2)cc(C)c1=O)c1c(F)c2c(c(-c3c(C)cccc3C)c1F)CCC2] |
| 451 | 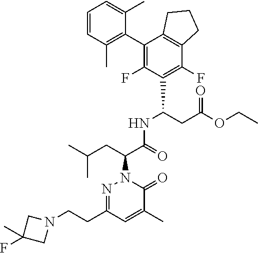 [SMILES: CCOC(=O)C[C@H](NC(=O)[C@H](CC(C)C)n1nc(CCN2CC(C)(F)C2)cc(C)c1=O)c1c(F)c2c(c(-c3c(C)cccc3C)c1F)CCC2] | 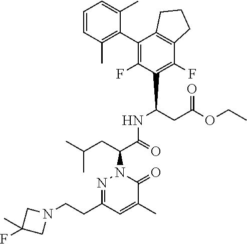 [SMILES: CCOC(=O)C[C@@H](NC(=O)[C@H](CC(C)C)n1nc(CCN2CC(C)(F)C2)cc(C)c1=O)c1c(F)c2c(c(-c3c(C)cccc3C)c1F)CCC2] |
| 452 | 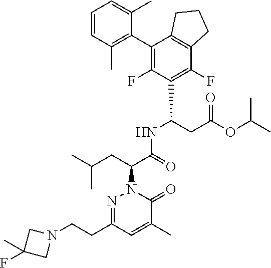 [SMILES: Cc1cccc(C)c1-c1c(F)c([C@H](CC(=O)OC(C)C)NC(=O)[C@H](CC(C)C)n2nc(CCN3CC(C)(F)C3)cc(C)c2=O)c(F)c2c1CCC2] | 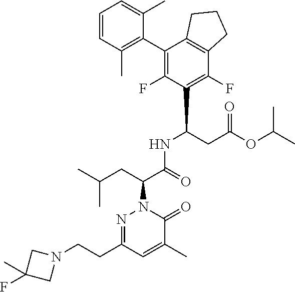 [SMILES: Cc1cccc(C)c1-c1c(F)c([C@@H](CC(=O)OC(C)C)NC(=O)[C@H](CC(C)C)n2nc(CCN3CC(C)(F)C3)cc(C)c2=O)c(F)c2c1CCC2] |
| 453 | 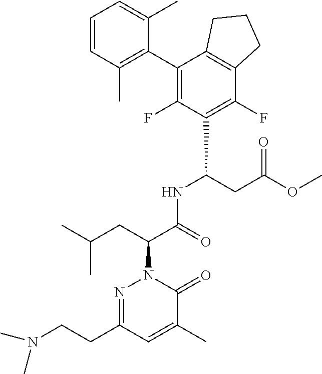 | 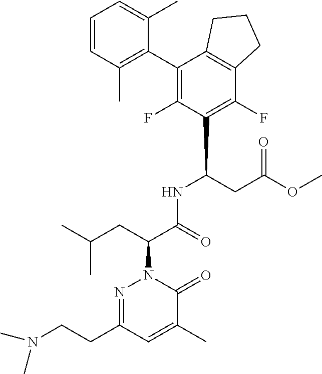 |
| 454 | 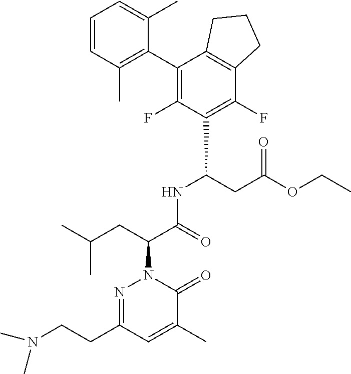 [SMILES: CCOC(=O)C[C@H](NC(=O)[C@H](CC(C)C)n1nc(CCN(C)C)cc(C)c1=O)c1c(F)c2c(c(-c3c(C)cccc3C)c1F)CCC2] | 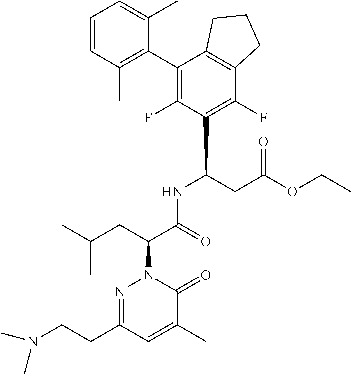 [SMILES: CCOC(=O)C[C@@H](NC(=O)[C@H](CC(C)C)n1nc(CCN(C)C)cc(C)c1=O)c1c(F)c2c(c(-c3c(C)cccc3C)c1F)CCC2] |
| 455 | 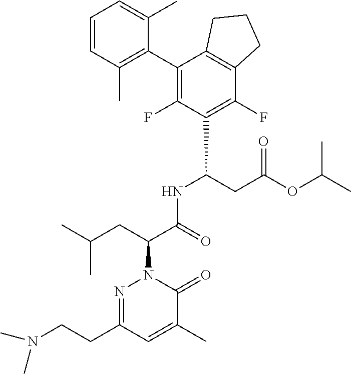 [SMILES: Cc1cccc(C)c1-c1c(F)c([C@H](CC(=O)OC(C)C)NC(=O)[C@H](CC(C)C)n2nc(CCN(C)C)cc(C)c2=O)c(F)c2c1CCC2] | 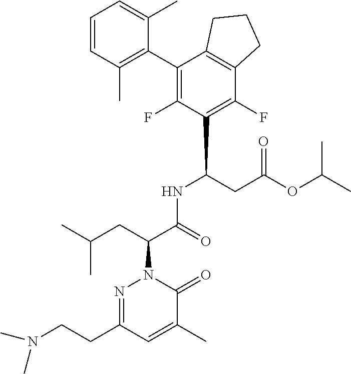 [SMILES: Cc1cccc(C)c1-c1c(F)c([C@@H](CC(=O)OC(C)C)NC(=O)[C@H](CC(C)C)n2nc(CCN(C)C)cc(C)c2=O)c(F)c2c1CCC2] |
| 456 | 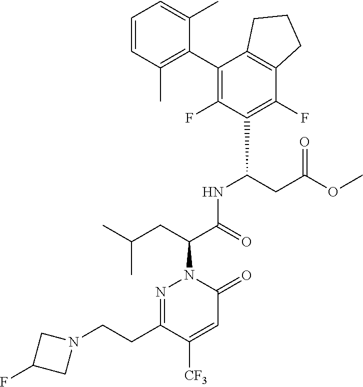 [SMILES: COC(=O)C[C@H](NC(=O)[C@H](CC(C)C)n1nc(CCN2CC(F)C2)c(C(F)(F)F)cc1=O)c1c(F)c2c(c(-c3c(C)cccc3C)c1F)CCC2] | 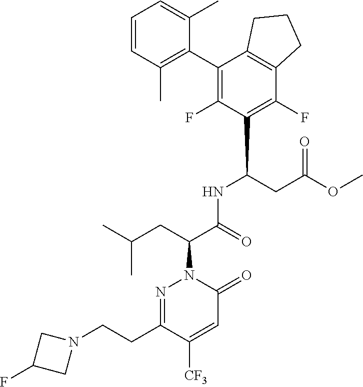 [SMILES: COC(=O)C[C@@H](NC(=O)[C@H](CC(C)C)n1nc(CCN2CC(F)C2)c(C(F)(F)F)cc1=O)c1c(F)c2c(c(-c3c(C)cccc3C)c1F)CCC2] |
| 457 | 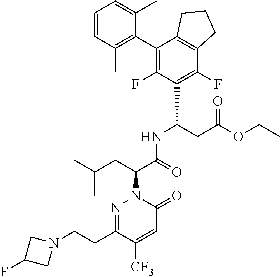 [SMILES: CCOC(=O)C[C@H](NC(=O)[C@H](CC(C)C)n1nc(CCN2CC(F)C2)c(C(F)(F)F)cc1=O)c1c(F)c2c(c(-c3c(C)cccc3C)c1F)CCC2] | 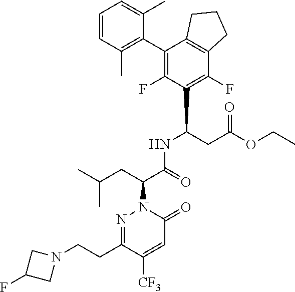 [SMILES: CCOC(=O)C[C@@H](NC(=O)[C@H](CC(C)C)n1nc(CCN2CC(F)C2)c(C(F)(F)F)cc1=O)c1c(F)c2c(c(-c3c(C)cccc3C)c1F)CCC2] |
| 458 | 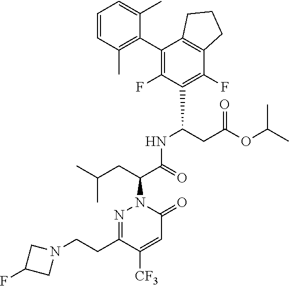 [SMILES: Cc1cccc(C)c1-c1c(F)c([C@H](CC(=O)OC(C)C)NC(=O)[C@H](CC(C)C)n2nc(CCN3CC(F)C3)c(C(F)(F)F)cc2=O)c(F)c2c1CCC2] | 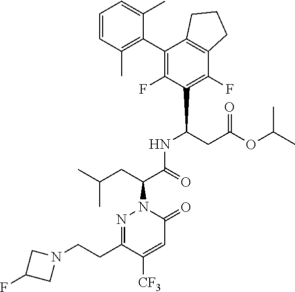 [SMILES: Cc1cccc(C)c1-c1c(F)c([C@@H](CC(=O)OC(C)C)NC(=O)[C@H](CC(C)C)n2nc(CCN3CC(F)C3)c(C(F)(F)F)cc2=O)c(F)c2c1CCC2] |
| 459 | 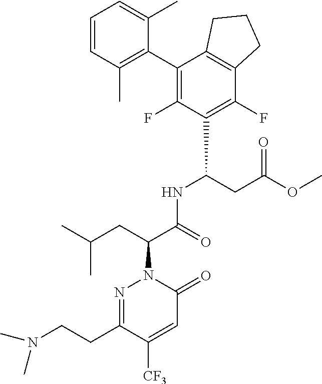 | 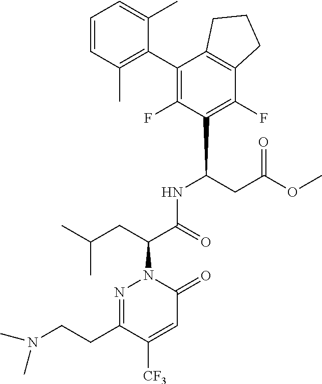 |
| 460 | 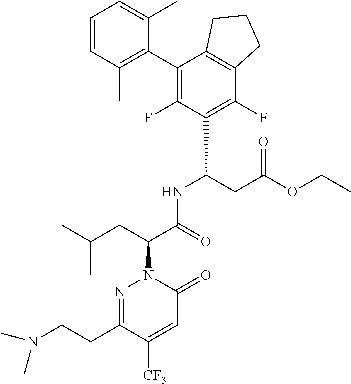 [SMILES: CCOC(=O)C[C@H](NC(=O)[C@H](CC(C)C)n1nc(CCN(C)C)c(C(F)(F)F)cc1=O)c1c(F)c2c(c(-c3c(C)cccc3C)c1F)CCC2] | 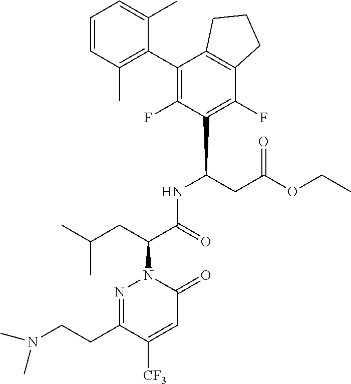 [SMILES: CCOC(=O)C[C@@H](NC(=O)[C@H](CC(C)C)n1nc(CCN(C)C)c(C(F)(F)F)cc1=O)c1c(F)c2c(c(-c3c(C)cccc3C)c1F)CCC2] |
| 461 | 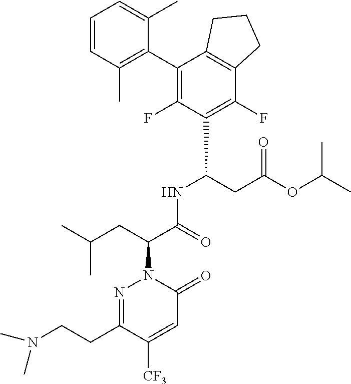 [SMILES: Cc1cccc(C)c1-c1c(F)c([C@H](CC(=O)OC(C)C)NC(=O)[C@H](CC(C)C)n2nc(CCN(C)C)c(C(F)(F)F)cc2=O)c(F)c2c1CCC2] | 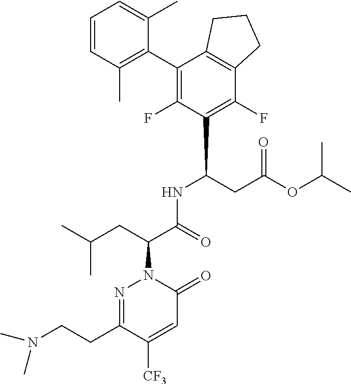 [SMILES: Cc1cccc(C)c1-c1c(F)c([C@@H](CC(=O)OC(C)C)NC(=O)[C@H](CC(C)C)n2nc(CCN(C)C)c(C(F)(F)F)cc2=O)c(F)c2c1CCC2] |

| | |
|---|---|
| 362 | 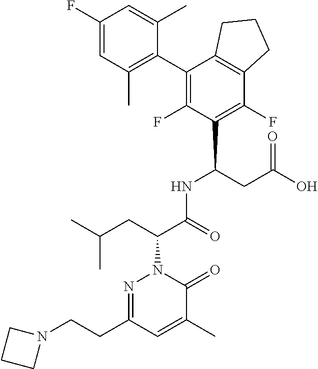 [SMILES: Cc1cc(F)cc(C)c1-c1c(F)c([C@@H](CC(=O)O)NC(=O)[C@@H](CC(C)C)n2nc(CCN3CCC3)cc(C)c2=O)c(F)c2c1CCC2] |
| 363 | 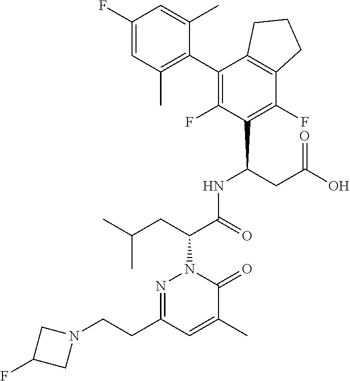 [SMILES: Cc1cc(F)cc(C)c1-c1c(F)c([C@@H](CC(=O)O)NC(=O)[C@@H](CC(C)C)n2nc(CCN3CC(F)C3)cc(C)c2=O)c(F)c2c1CCC2] |
| 364 | 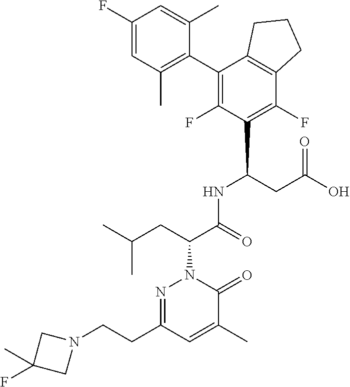 [SMILES: Cc1cc(F)cc(C)c1-c1c(F)c([C@@H](CC(=O)O)NC(=O)[C@@H](CC(C)C)n2nc(CCN3CC(C)(F)C3)cc(C)c2=O)c(F)c2c1CCC2] |
| 365 | 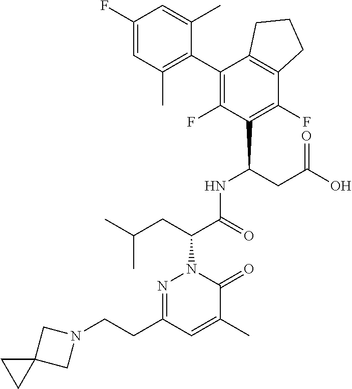 [SMILES: Cc1cc(F)cc(C)c1-c1c(F)c([C@@H](CC(=O)O)NC(=O)[C@@H](CC(C)C)n2nc(CCN3CC4(CC4)C3)cc(C)c2=O)c(F)c2c1CCC2] |
| 366 | 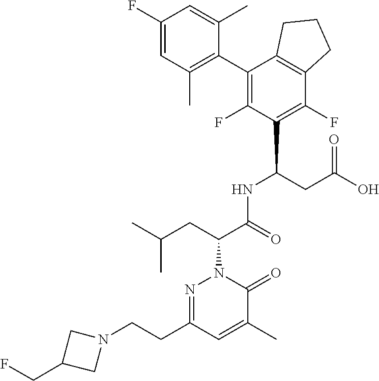 [SMILES: Cc1cc(F)cc(C)c1-c1c(F)c([C@@H](CC(=O)O)NC(=O)[C@@H](CC(C)C)n2nc(CCN3CC(CF)C3)cc(C)c2=O)c(F)c2c1CCC2] |
| 367 | 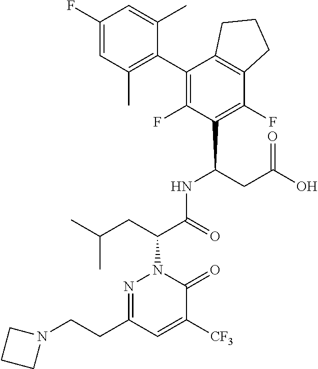 [SMILES: Cc1cc(F)cc(C)c1-c1c(F)c([C@@H](CC(=O)O)NC(=O)[C@@H](CC(C)C)n2nc(CCN3CCC3)cc(C(F)(F)F)c2=O)c(F)c2c1CCC2] |
| 368 | 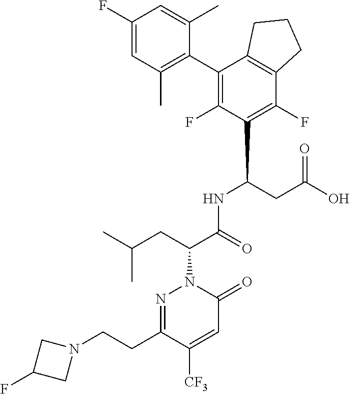 [SMILES: Cc1cc(F)cc(C)c1-c1c(F)c([C@@H](CC(=O)O)NC(=O)[C@@H](CC(C)C)n2nc(CCN3CC(F)C3)c(C(F)(F)F)cc2=O)c(F)c2c1CCC2] |
| 369 | 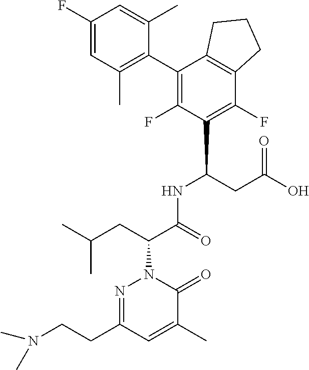 [SMILES: Cc1cc(F)cc(C)c1-c1c(F)c([C@@H](CC(=O)O)NC(=O)[C@@H](CC(C)C)n2nc(CCN(C)C)cc(C)c2=O)c(F)c2c1CCC2] |
| 370 | 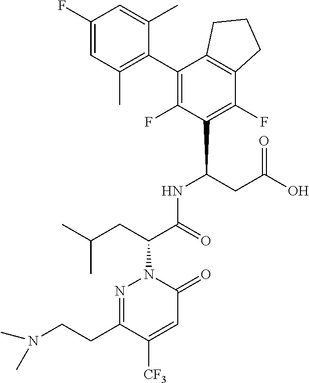 [SMILES: Cc1cc(F)cc(C)c1-c1c(F)c([C@@H](CC(=O)O)NC(=O)[C@@H](CC(C)C)n2nc(CCN(C)C)c(C(F)(F)F)cc2=O)c(F)c2c1CCC2] |
| 371 | 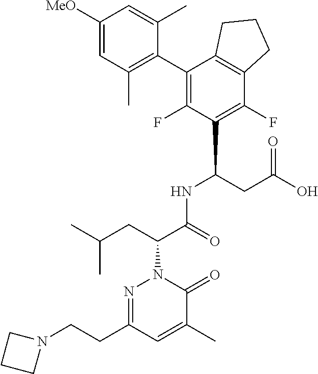 [SMILES: COc1cc(C)c(-c2c(F)c([C@@H](CC(=O)O)NC(=O)[C@@H](CC(C)C)n3nc(CCN4CCC4)cc(C)c3=O)c(F)c3c2CCC3)c(C)c1] |
| 372 | 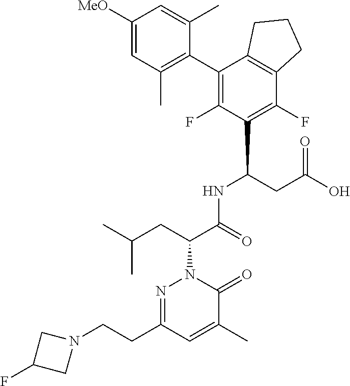 [SMILES: COc1cc(C)c(-c2c(F)c([C@@H](CC(=O)O)NC(=O)[C@@H](CC(C)C)n3nc(CCN4CC(F)C4)cc(C)c3=O)c(F)c3c2CCC3)c(C)c1] |
| 373 | 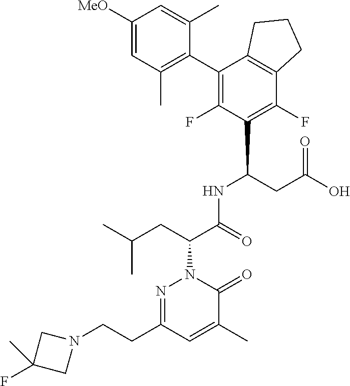 [SMILES: COc1cc(C)c(-c2c(F)c([C@@H](CC(=O)O)NC(=O)[C@@H](CC(C)C)n3nc(CCN4CC(C)(F)C4)cc(C)c3=O)c(F)c3c2CCC3)c(C)c1] |
| 374 | 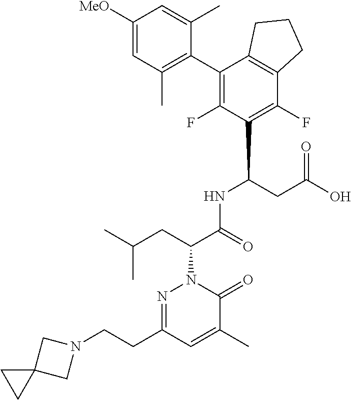 [SMILES: COc1cc(C)c(-c2c(F)c([C@@H](CC(=O)O)NC(=O)[C@@H](CC(C)C)n3nc(CCN4CC5(CC5)C4)cc(C)c3=O)c(F)c3c2CCC3)c(C)c1] |
| 375 | 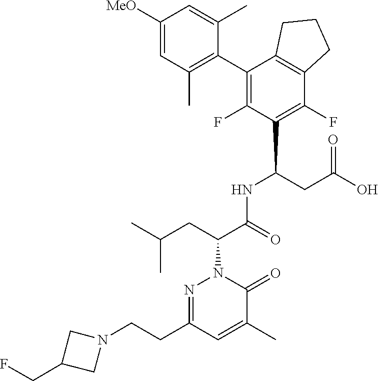 [SMILES: COc1cc(C)c(-c2c(F)c([C@@H](CC(=O)O)NC(=O)[C@@H](CC(C)C)n3nc(CCN4CC(CF)C4)cc(C)c3=O)c(F)c3c2CCC3)c(C)c1] |
| 376 | 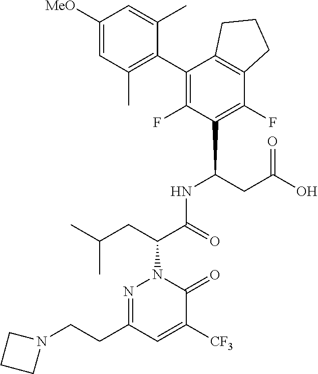 [SMILES: COc1cc(C)c(-c2c(F)c([C@@H](CC(=O)O)NC(=O)[C@@H](CC(C)C)n3nc(CCN4CCC4)cc(C(F)(F)F)c3=O)c(F)c3c2CCC3)c(C)c1] |
| 377 | 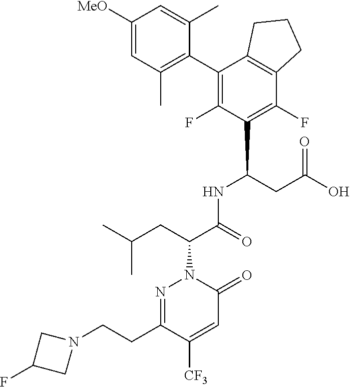 [SMILES: COc1cc(C)c(-c2c(F)c([C@@H](CC(=O)O)NC(=O)[C@@H](CC(C)C)n3nc(CCN4CC(F)C4)c(C(F)(F)F)cc3=O)c(F)c3c2CCC3)c(C)c1] |
| 378 | 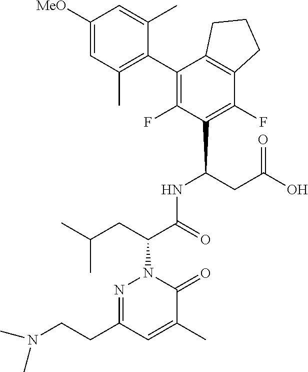 [SMILES: COc1cc(C)c(-c2c(F)c([C@@H](CC(=O)O)NC(=O)[C@@H](CC(C)C)n3nc(CCN(C)C)cc(C)c3=O)c(F)c3c2CCC3)c(C)c1] |
| 379 | 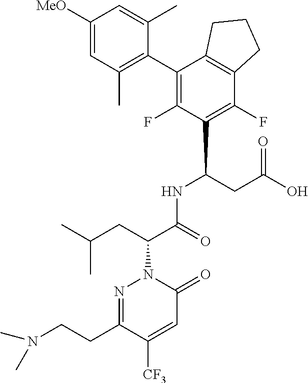 [SMILES: COc1cc(C)c(-c2c(F)c([C@@H](CC(=O)O)NC(=O)[C@@H](CC(C)C)n3nc(CCN(C)C)c(C(F)(F)F)cc3=O)c(F)c3c2CCC3)c(C)c1] |
| 380 | 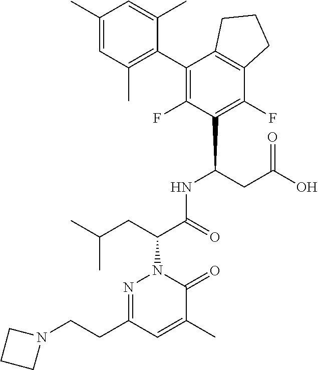 [SMILES: Cc1cc(C)c(-c2c(F)c([C@@H](CC(=O)O)NC(=O)[C@@H](CC(C)C)n3nc(CCN4CCC4)cc(C)c3=O)c(F)c3c2CCC3)c(C)c1] |
| 381 | 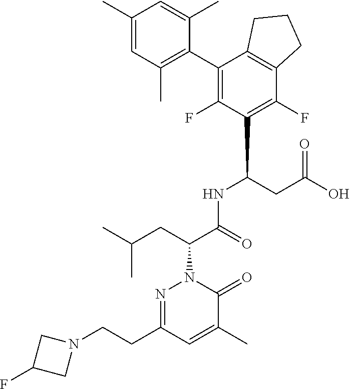 [SMILES: Cc1cc(C)c(-c2c(F)c([C@@H](CC(=O)O)NC(=O)[C@@H](CC(C)C)n3nc(CCN4CC(F)C4)cc(C)c3=O)c(F)c3c2CCC3)c(C)c1] |
| 382 | 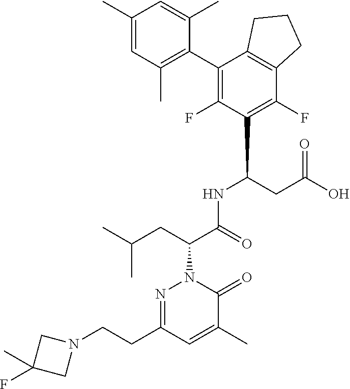 [SMILES: Cc1cc(C)c(-c2c(F)c([C@@H](CC(=O)O)NC(=O)[C@@H](CC(C)C)n3nc(CCN4CC(C)(F)C4)cc(C)c3=O)c(F)c3c2CCC3)c(C)c1] |
| 383 | 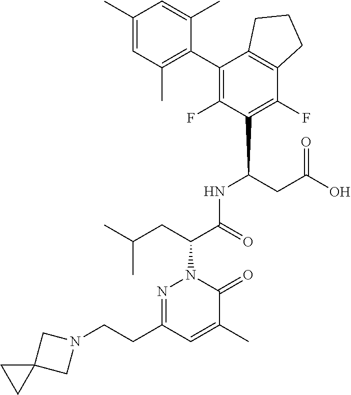 [SMILES: Cc1cc(C)c(-c2c(F)c([C@@H](CC(=O)O)NC(=O)[C@@H](CC(C)C)n3nc(CCN4CC5(CC5)C4)cc(C)c3=O)c(F)c3c2CCC3)c(C)c1] |
| 384 | 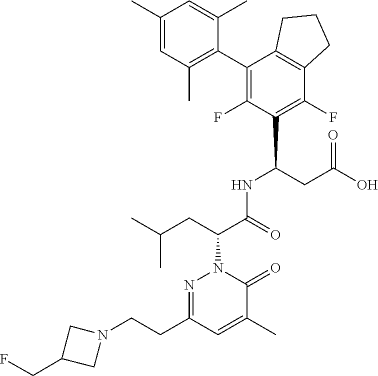 [SMILES: Cc1cc(C)c(-c2c(F)c([C@@H](CC(=O)O)NC(=O)[C@@H](CC(C)C)n3nc(CCN4CC(CF)C4)cc(C)c3=O)c(F)c3c2CCC3)c(C)c1] |
| 385 | 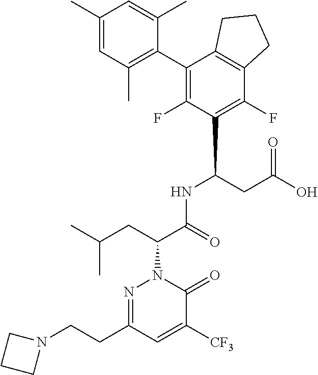 [SMILES: Cc1cc(C)c(-c2c(F)c([C@@H](CC(=O)O)NC(=O)[C@@H](CC(C)C)n3nc(CCN4CCC4)cc(C(F)(F)F)c3=O)c(F)c3c2CCC3)c(C)c1] |
| 386 | 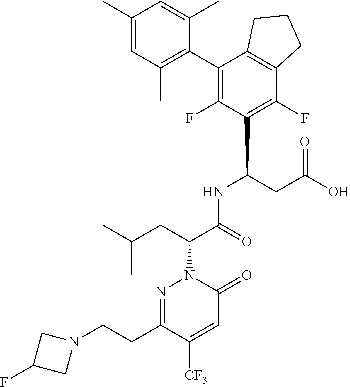 [SMILES: Cc1cc(C)c(-c2c(F)c([C@@H](CC(=O)O)NC(=O)[C@@H](CC(C)C)n3nc(CCN4CC(F)C4)c(C(F)(F)F)cc3=O)c(F)c3c2CCC3)c(C)c1] |
| 387 | 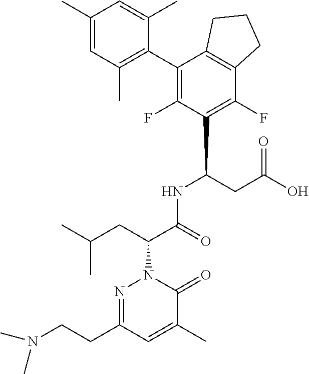 [SMILES: Cc1cc(C)c(-c2c(F)c([C@@H](CC(=O)O)NC(=O)[C@@H](CC(C)C)n3nc(CCN(C)C)cc(C)c3=O)c(F)c3c2CCC3)c(C)c1] |
| 388 | 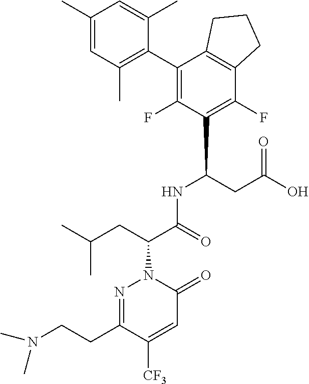 [SMILES: Cc1cc(C)c(-c2c(F)c([C@@H](CC(=O)O)NC(=O)[C@@H](CC(C)C)n3nc(CCN(C)C)c(C(F)(F)F)cc3=O)c(F)c3c2CCC3)c(C)c1] |
| 389 | 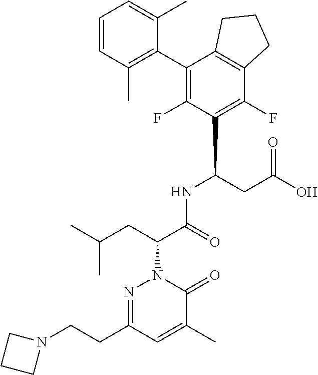 [SMILES: Cc1cccc(C)c1-c1c(F)c([C@@H](CC(=O)O)NC(=O)[C@@H](CC(C)C)n2nc(CCN3CCC3)cc(C)c2=O)c(F)c2c1CCC2] |
| 390 | 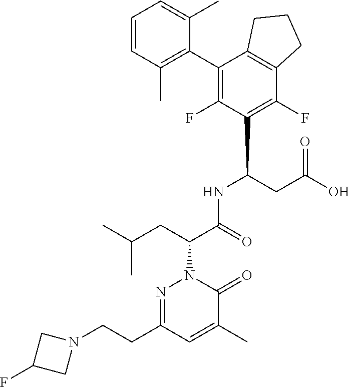 [SMILES: Cc1cccc(C)c1-c1c(F)c([C@@H](CC(=O)O)NC(=O)[C@@H](CC(C)C)n2nc(CCN3CC(F)C3)cc(C)c2=O)c(F)c2c1CCC2] |
| 391 | 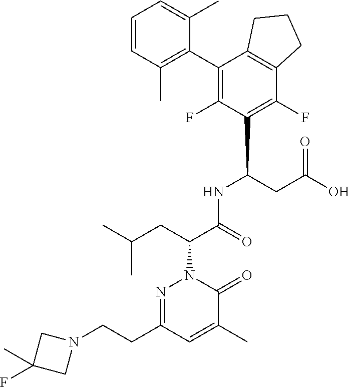 [SMILES: Cc1cccc(C)c1-c1c(F)c([C@@H](CC(=O)O)NC(=O)[C@@H](CC(C)C)n2nc(CCN3CC(C)(F)C3)cc(C)c2=O)c(F)c2c1CCC2] |
| 392 | 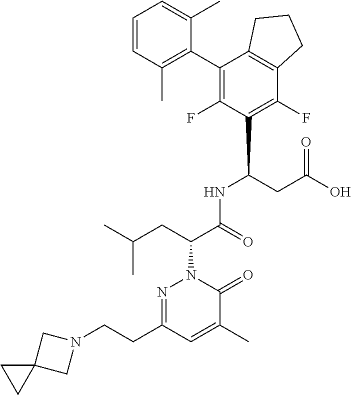 [SMILES: Cc1cccc(C)c1-c1c(F)c([C@@H](CC(=O)O)NC(=O)[C@@H](CC(C)C)n2nc(CCN3CC4(CC4)C3)cc(C)c2=O)c(F)c2c1CCC2] |
| 393 | 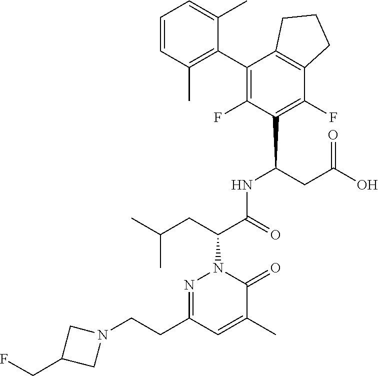 [SMILES: Cc1cccc(C)c1-c1c(F)c([C@@H](CC(=O)O)NC(=O)[C@@H](CC(C)C)n2nc(CCN3CC(CF)C3)cc(C)c2=O)c(F)c2c1CCC2] |
| 394 | 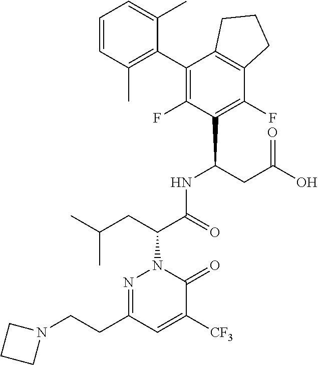 [SMILES: Cc1cccc(C)c1-c1c(F)c([C@@H](CC(=O)O)NC(=O)[C@@H](CC(C)C)n2nc(CCN3CCC3)cc(C(F)(F)F)c2=O)c(F)c2c1CCC2] |
| 395 | 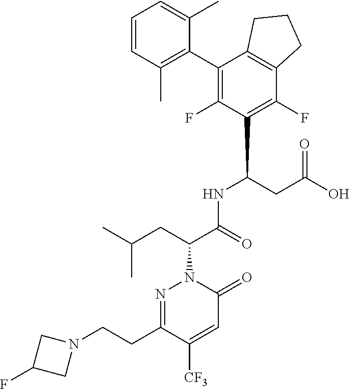 [SMILES: Cc1cccc(C)c1-c1c(F)c([C@@H](CC(=O)O)NC(=O)[C@@H](CC(C)C)n2nc(CCN3CC(F)C3)c(C(F)(F)F)cc2=O)c(F)c2c1CCC2] |
| 396 | 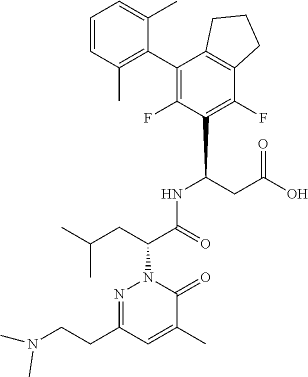 [SMILES: Cc1cccc(C)c1-c1c(F)c([C@@H](CC(=O)O)NC(=O)[C@@H](CC(C)C)n2nc(CCN(C)C)cc(C)c2=O)c(F)c2c1CCC2] |
| 397 | 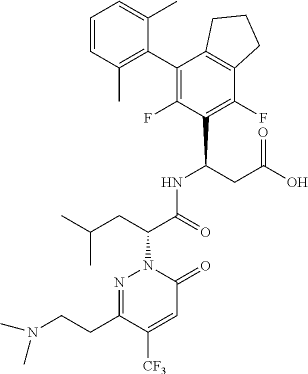 [SMILES: Cc1cccc(C)c1-c1c(F)c([C@@H](CC(=O)O)NC(=O)[C@@H](CC(C)C)n2nc(CCN(C)C)c(C(F)(F)F)cc2=O)c(F)c2c1CCC2] |
| 398 | 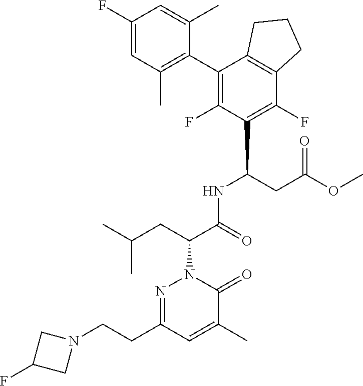 [SMILES: COC(=O)C[C@@H](NC(=O)[C@@H](CC(C)C)n1nc(CCN2CC(F)C2)cc(C)c1=O)c1c(F)c2c(c(-c3c(C)cc(F)cc3C)c1F)CCC2] |
| 399 | 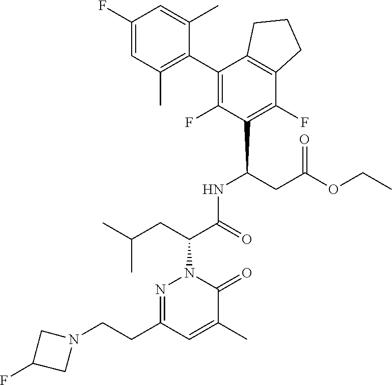 [SMILES: CCOC(=O)C[C@@H](NC(=O)[C@@H](CC(C)C)n1nc(CCN2CC(F)C2)cc(C)c1=O)c1c(F)c2c(c(-c3c(C)cc(F)cc3C)c1F)CCC2] |
| 400 | 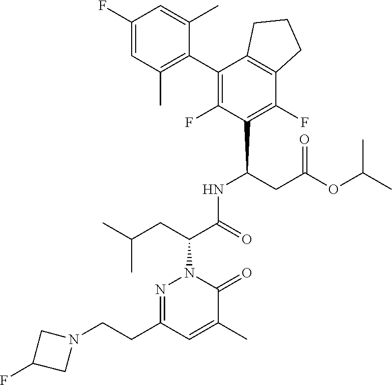 [SMILES: Cc1cc(F)cc(C)c1-c1c(F)c([C@@H](CC(=O)OC(C)C)NC(=O)[C@@H](CC(C)C)n2nc(CCN3CC(F)C3)cc(C)c2=O)c(F)c2c1CCC2] |
| 401 | 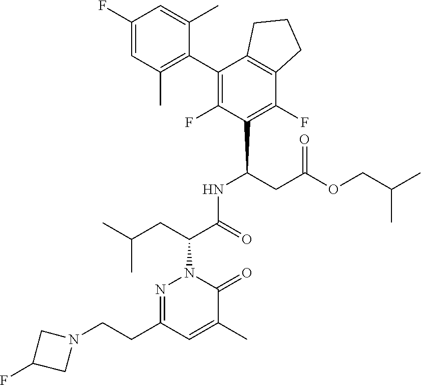 [SMILES: Cc1cc(F)cc(C)c1-c1c(F)c([C@@H](CC(=O)OCC(C)C)NC(=O)[C@@H](CC(C)C)n2nc(CCN3CC(F)C3)cc(C)c2=O)c(F)c2c1CCC2] |
| 402 | 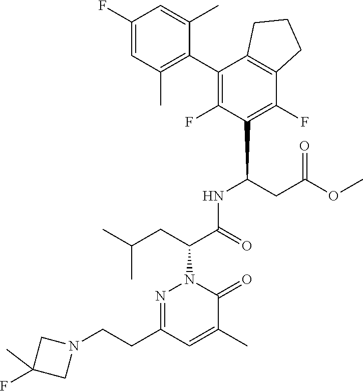 [SMILES: COC(=O)C[C@@H](NC(=O)[C@@H](CC(C)C)n1nc(CCN2CC(C)(F)C2)cc(C)c1=O)c1c(F)c2c(c(-c3c(C)cc(F)cc3C)c1F)CCC2] |
| 403 | 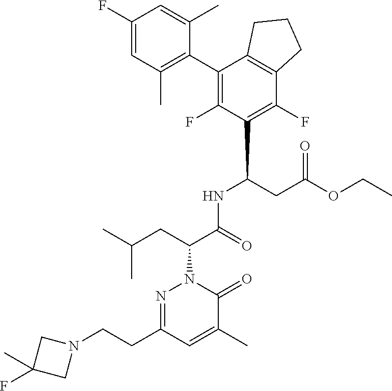 [SMILES: CCOC(=O)C[C@@H](NC(=O)[C@@H](CC(C)C)n1nc(CCN2CC(C)(F)C2)cc(C)c1=O)c1c(F)c2c(c(-c3c(C)cc(F)cc3C)c1F)CCC2] |
| 404 | 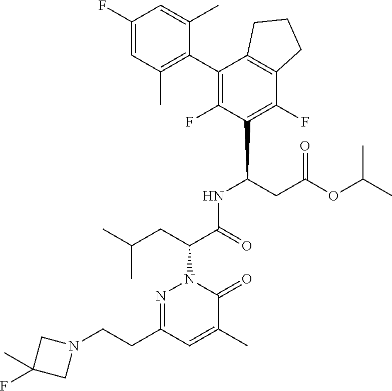 [SMILES: Cc1cc(F)cc(C)c1-c1c(F)c([C@@H](CC(=O)OC(C)C)NC(=O)[C@@H](CC(C)C)n2nc(CCN3CC(C)(F)C3)cc(C)c2=O)c(F)c2c1CCC2] |
| 405 | 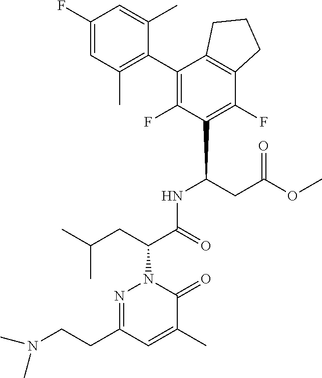 |
| 406 | 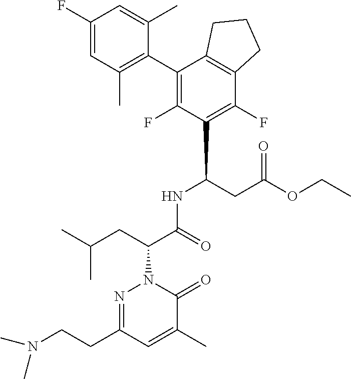 [SMILES: CCOC(=O)C[C@@H](NC(=O)[C@@H](CC(C)C)n1nc(CCN(C)C)cc(C)c1=O)c1c(F)c2c(c(-c3c(C)cc(F)cc3C)c1F)CCC2] |
| 407 | 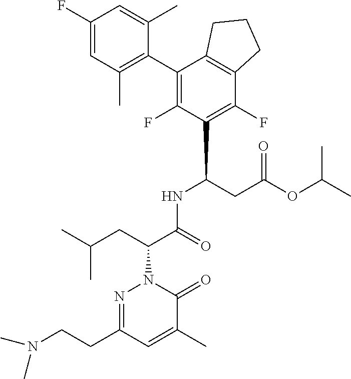 [SMILES: Cc1cc(F)cc(C)c1-c1c(F)c([C@@H](CC(=O)OC(C)C)NC(=O)[C@@H](CC(C)C)n2nc(CCN(C)C)cc(C)c2=O)c(F)c2c1CCC2] |
| 408 | 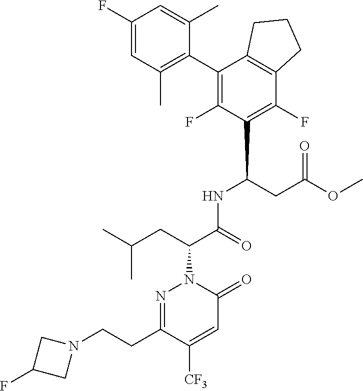 [SMILES: COC(=O)C[C@@H](NC(=O)[C@@H](CC(C)C)n1nc(CCN2CC(F)C2)c(C(F)(F)F)cc1=O)c1c(F)c2c(c(-c3c(C)cc(F)cc3C)c1F)CCC2] |
| 409 | 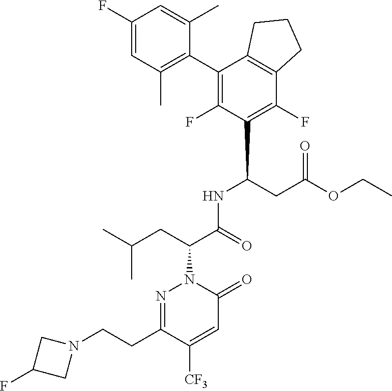 [SMILES: CCOC(=O)C[C@@H](NC(=O)[C@@H](CC(C)C)n1nc(CCN2CC(F)C2)c(C(F)(F)F)cc1=O)c1c(F)c2c(c(-c3c(C)cc(F)cc3C)c1F)CCC2] |
| 410 | 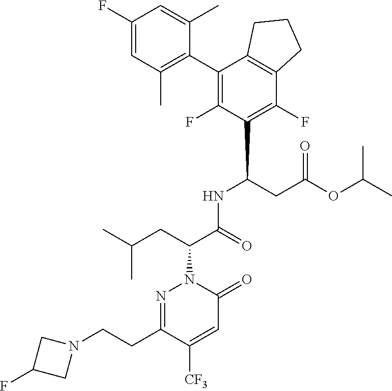 [SMILES: Cc1cc(F)cc(C)c1-c1c(F)c([C@@H](CC(=O)OC(C)C)NC(=O)[C@@H](CC(C)C)n2nc(CCN3CC(F)C3)c(C(F)(F)F)cc2=O)c(F)c2c1CCC2] |
| 411 | 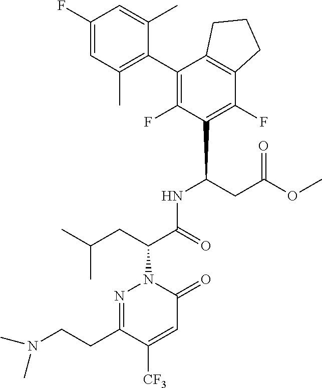 |
| 412 | 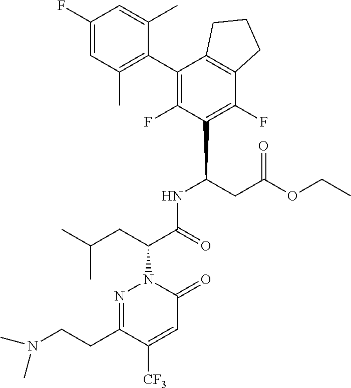 [SMILES: CCOC(=O)C[C@@H](NC(=O)[C@@H](CC(C)C)n1nc(CCN(C)C)c(C(F)(F)F)cc1=O)c1c(F)c2c(c(-c3c(C)cc(F)cc3C)c1F)CCC2] |
| 413 | 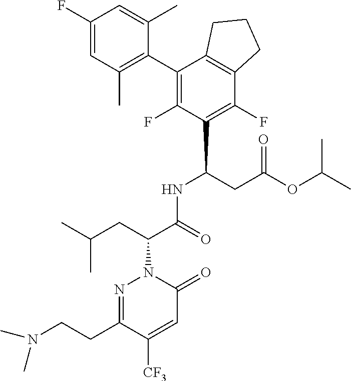 [SMILES: Cc1cc(F)cc(C)c1-c1c(F)c([C@@H](CC(=O)OC(C)C)NC(=O)[C@@H](CC(C)C)n2nc(CCN(C)C)c(C(F)(F)F)cc2=O)c(F)c2c1CCC2] |
| 414 | 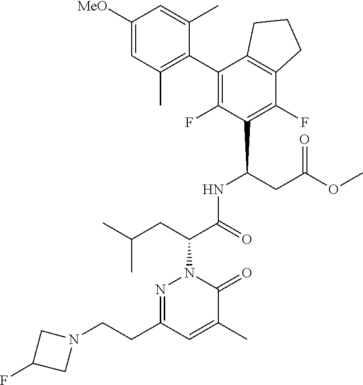 [SMILES: COC(=O)C[C@@H](NC(=O)[C@@H](CC(C)C)n1nc(CCN2CC(F)C2)cc(C)c1=O)c1c(F)c2c(c(-c3c(C)cc(OC)cc3C)c1F)CCC2] |
| 415 | 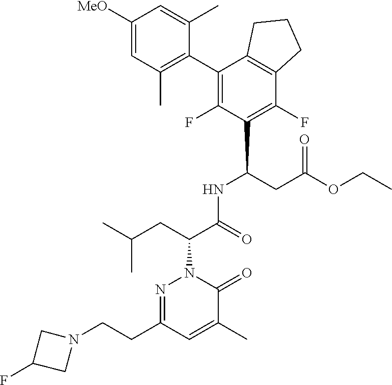 [SMILES: CCOC(=O)C[C@@H](NC(=O)[C@@H](CC(C)C)n1nc(CCN2CC(F)C2)cc(C)c1=O)c1c(F)c2c(c(-c3c(C)cc(OC)cc3C)c1F)CCC2] |
| 416 | 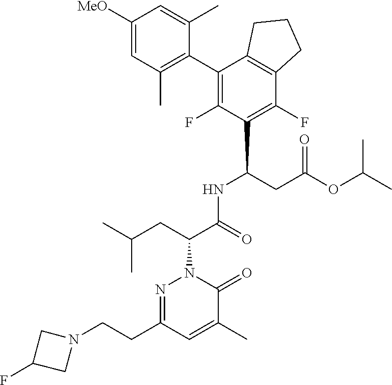 [SMILES: COc1cc(C)c(-c2c(F)c([C@@H](CC(=O)OC(C)C)NC(=O)[C@@H](CC(C)C)n3nc(CCN4CC(F)C4)cc(C)c3=O)c(F)c3c2CCC3)c(C)c1] |
| 417 | 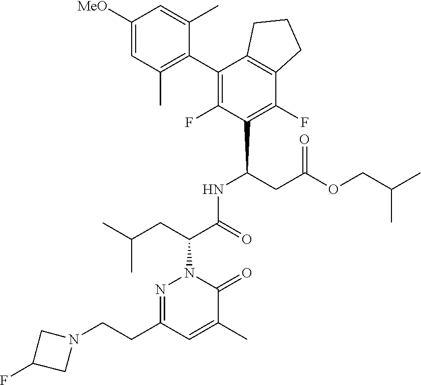 [SMILES: COc1cc(C)c(-c2c(F)c([C@@H](CC(=O)OCC(C)C)NC(=O)[C@@H](CC(C)C)n3nc(CCN4CC(F)C4)cc(C)c3=O)c(F)c3c2CCC3)c(C)c1] |
| 418 | 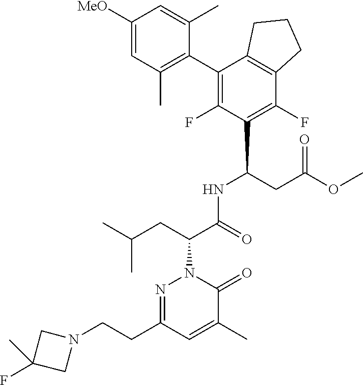 [SMILES: COC(=O)C[C@@H](NC(=O)[C@@H](CC(C)C)n1nc(CCN2CC(C)(F)C2)cc(C)c1=O)c1c(F)c2c(c(-c3c(C)cc(OC)cc3C)c1F)CCC2] |
| 419 | 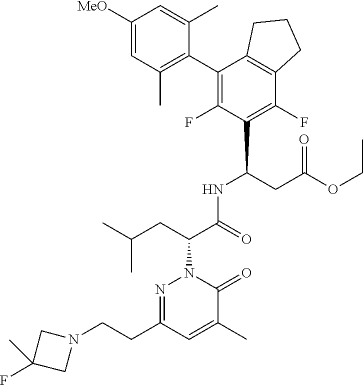 [SMILES: CCOC(=O)C[C@@H](NC(=O)[C@@H](CC(C)C)n1nc(CCN2CC(C)(F)C2)cc(C)c1=O)c1c(F)c2c(c(-c3c(C)cc(OC)cc3C)c1F)CCC2] |
| 420 | 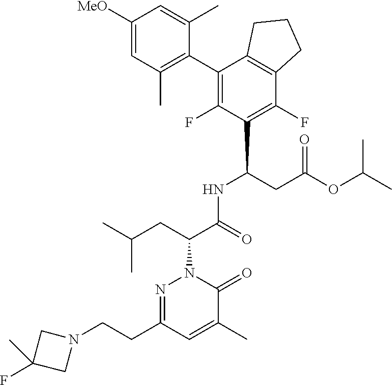 [SMILES: COc1cc(C)c(-c2c(F)c([C@@H](CC(=O)OC(C)C)NC(=O)[C@@H](CC(C)C)n3nc(CCN4CC(C)(F)C4)cc(C)c3=O)c(F)c3c2CCC3)c(C)c1] |
| 421 | 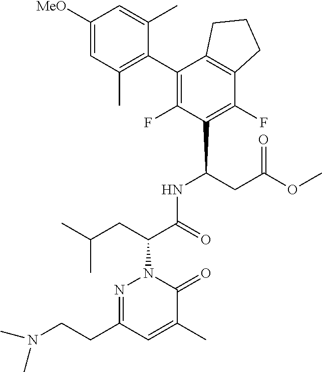 |
| 422 | 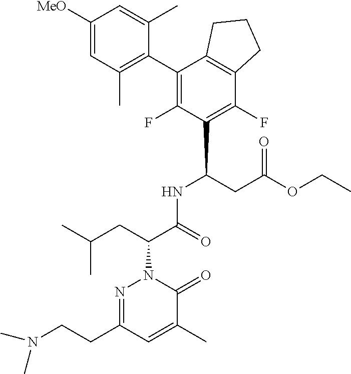 [SMILES: CCOC(=O)C[C@@H](NC(=O)[C@@H](CC(C)C)n1nc(CCN(C)C)cc(C)c1=O)c1c(F)c2c(c(-c3c(C)cc(OC)cc3C)c1F)CCC2] |
| 423 | 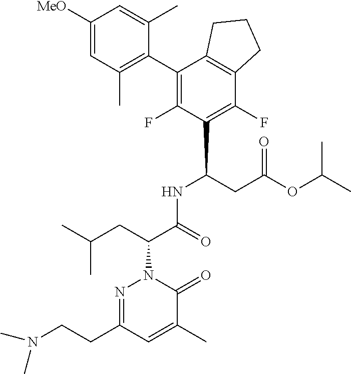 [SMILES: COc1cc(C)c(-c2c(F)c([C@@H](CC(=O)OC(C)C)NC(=O)[C@@H](CC(C)C)n3nc(CCN(C)C)cc(C)c3=O)c(F)c3c2CCC3)c(C)c1] |
| 424 | 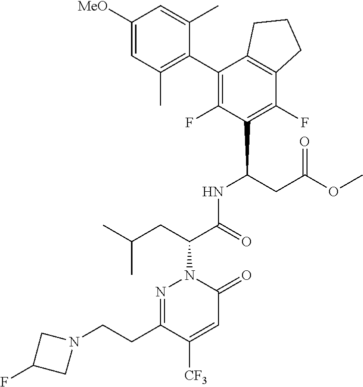 [SMILES: COC(=O)C[C@@H](NC(=O)[C@@H](CC(C)C)n1nc(CCN2CC(F)C2)c(C(F)(F)F)cc1=O)c1c(F)c2c(c(-c3c(C)cc(OC)cc3C)c1F)CCC2] |
| 425 | 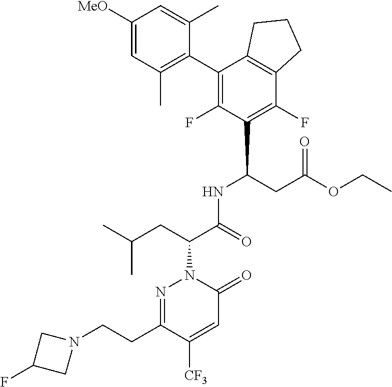 [SMILES: CCOC(=O)C[C@@H](NC(=O)[C@@H](CC(C)C)n1nc(CCN2CC(F)C2)c(C(F)(F)F)cc1=O)c1c(F)c2c(c(-c3c(C)cc(OC)cc3C)c1F)CCC2] |
| 426 | 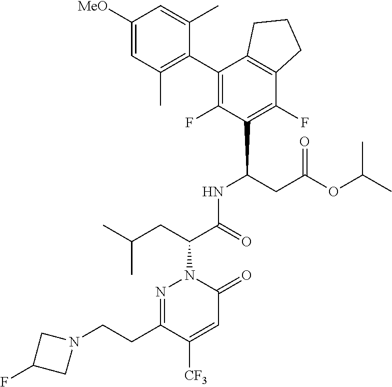 [SMILES: COc1cc(C)c(-c2c(F)c([C@@H](CC(=O)OC(C)C)NC(=O)[C@@H](CC(C)C)n3nc(CCN4CC(F)C4)c(C(F)(F)F)cc3=O)c(F)c3c2CCC3)c(C)c1] |
| 427 | 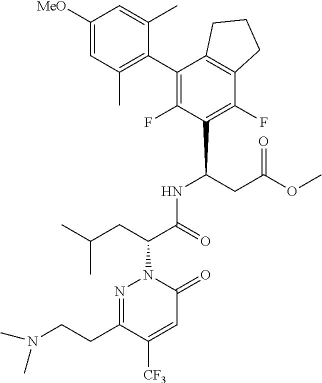 |
| 428 | 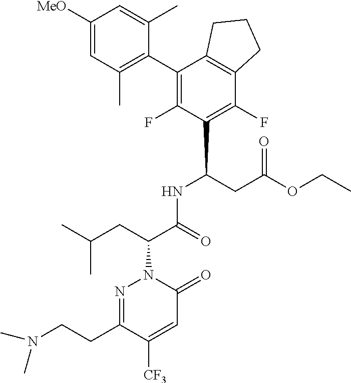 [SMILES: CCOC(=O)C[C@@H](NC(=O)[C@@H](CC(C)C)n1nc(CCN(C)C)c(C(F)(F)F)cc1=O)c1c(F)c2c(c(-c3c(C)cc(OC)cc3C)c1F)CCC2] |
| 429 | 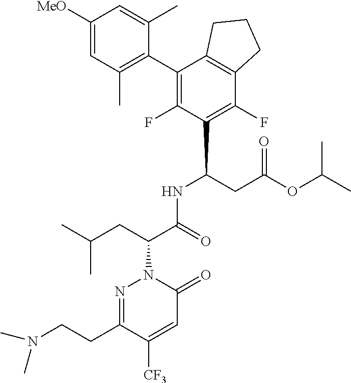 [SMILES: COc1cc(C)c(-c2c(F)c([C@@H](CC(=O)OC(C)C)NC(=O)[C@@H](CC(C)C)n3nc(CCN(C)C)c(C(F)(F)F)cc3=O)c(F)c3c2CCC3)c(C)c1] |
| 430 | 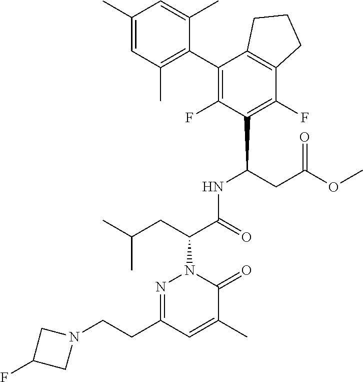 [SMILES: COC(=O)C[C@@H](NC(=O)[C@@H](CC(C)C)n1nc(CCN2CC(F)C2)cc(C)c1=O)c1c(F)c2c(c(-c3c(C)cc(C)cc3C)c1F)CCC2] |
| 431 | 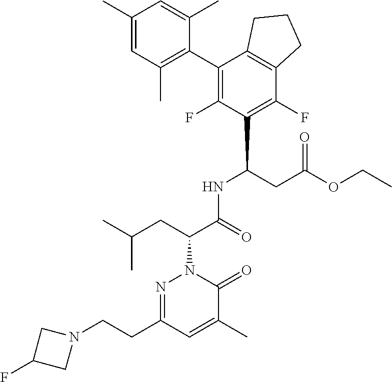 [SMILES: CCOC(=O)C[C@@H](NC(=O)[C@@H](CC(C)C)n1nc(CCN2CC(F)C2)cc(C)c1=O)c1c(F)c2c(c(-c3c(C)cc(C)cc3C)c1F)CCC2] |
| 432 | 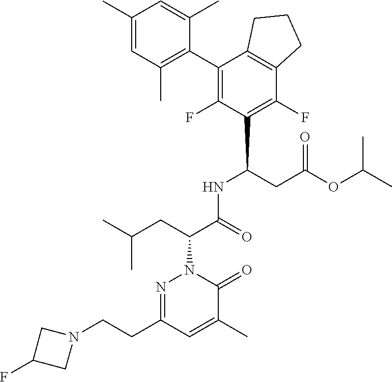 [SMILES: Cc1cc(C)c(-c2c(F)c([C@@H](CC(=O)OC(C)C)NC(=O)[C@@H](CC(C)C)n3nc(CCN4CC(F)C4)cc(C)c3=O)c(F)c3c2CCC3)c(C)c1] |
| 433 | 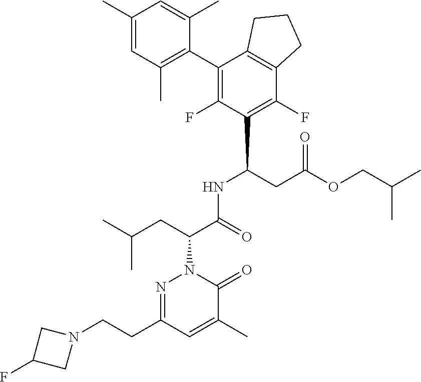 [SMILES: Cc1cc(C)c(-c2c(F)c([C@@H](CC(=O)OCC(C)C)NC(=O)[C@@H](CC(C)C)n3nc(CCN4CC(F)C4)cc(C)c3=O)c(F)c3c2CCC3)c(C)c1] |
| 434 | 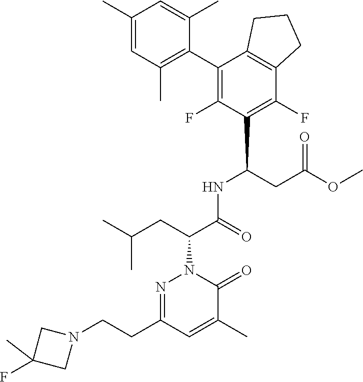 [SMILES: COC(=O)C[C@@H](NC(=O)[C@@H](CC(C)C)n1nc(CCN2CC(C)(F)C2)cc(C)c1=O)c1c(F)c2c(c(-c3c(C)cc(C)cc3C)c1F)CCC2] |
| 435 | 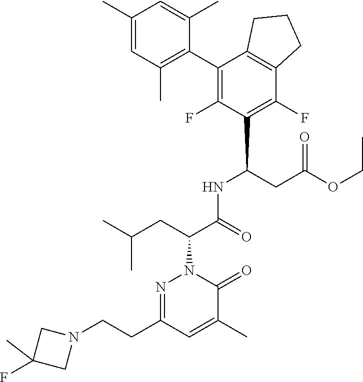 [SMILES: CCOC(=O)C[C@@H](NC(=O)[C@@H](CC(C)C)n1nc(CCN2CC(C)(F)C2)cc(C)c1=O)c1c(F)c2c(c(-c3c(C)cc(C)cc3C)c1F)CCC2] |
| 436 | 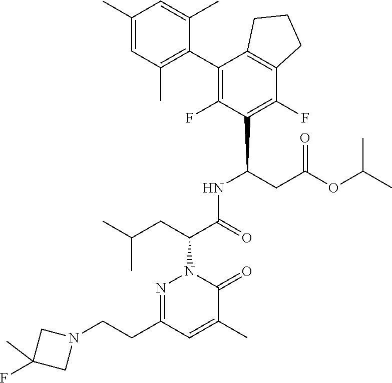 [SMILES: Cc1cc(C)c(-c2c(F)c([C@@H](CC(=O)OC(C)C)NC(=O)[C@@H](CC(C)C)n3nc(CCN4CC(C)(F)C4)cc(C)c3=O)c(F)c3c2CCC3)c(C)c1] |
| 437 | 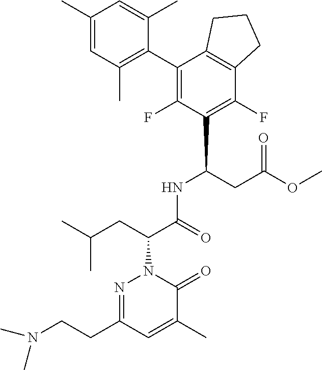 |
| 438 | 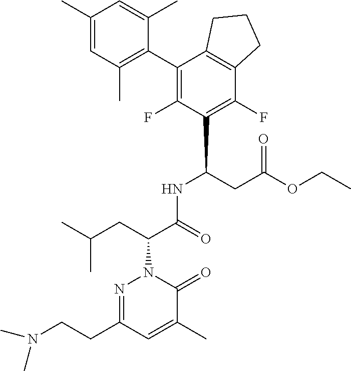 [SMILES: CCOC(=O)C[C@@H](NC(=O)[C@@H](CC(C)C)n1nc(CCN(C)C)cc(C)c1=O)c1c(F)c2c(c(-c3c(C)cc(C)cc3C)c1F)CCC2] |
| 439 | 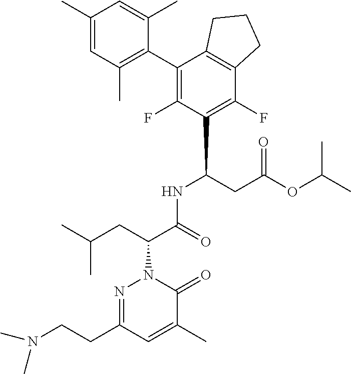 [SMILES: Cc1cc(C)c(-c2c(F)c([C@@H](CC(=O)OC(C)C)NC(=O)[C@@H](CC(C)C)n3nc(CCN(C)C)cc(C)c3=O)c(F)c3c2CCC3)c(C)c1] |
| 440 | 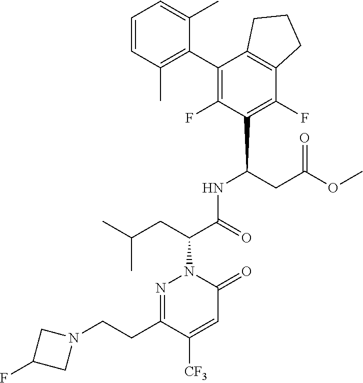 [SMILES: COC(=O)C[C@@H](NC(=O)[C@@H](CC(C)C)n1nc(CCN2CC(F)C2)c(C(F)(F)F)cc1=O)c1c(F)c2c(c(-c3c(C)cccc3C)c1F)CCC2] |
| 441 | 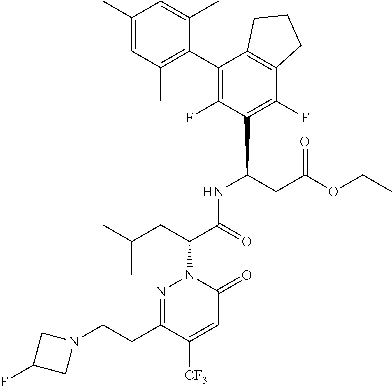 [SMILES: CCOC(=O)C[C@@H](NC(=O)[C@@H](CC(C)C)n1nc(CCN2CC(F)C2)c(C(F)(F)F)cc1=O)c1c(F)c2c(c(-c3c(C)cc(C)cc3C)c1F)CCC2] |
| 442 | 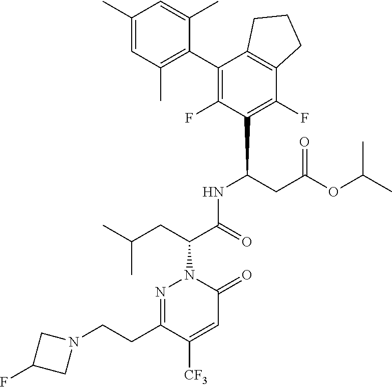 [SMILES: Cc1cc(C)c(-c2c(F)c([C@@H](CC(=O)OC(C)C)NC(=O)[C@@H](CC(C)C)n3nc(CCN4CC(F)C4)c(C(F)(F)F)cc3=O)c(F)c3c2CCC3)c(C)c1] |
| 443 | 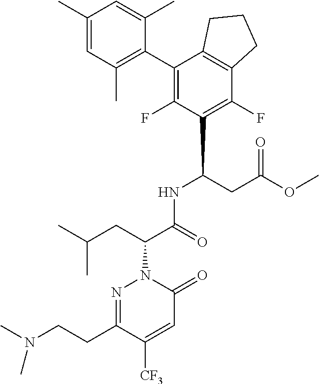 |
| 444 | 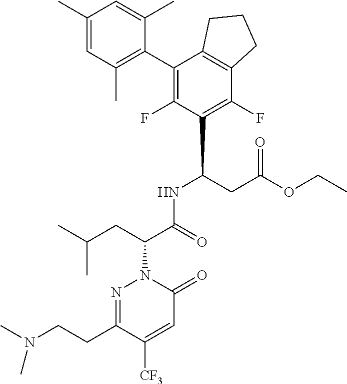 [SMILES: CCOC(=O)C[C@@H](NC(=O)[C@@H](CC(C)C)n1nc(CCN(C)C)c(C(F)(F)F)cc1=O)c1c(F)c2c(c(-c3c(C)cc(C)cc3C)c1F)CCC2] |
| 445 | 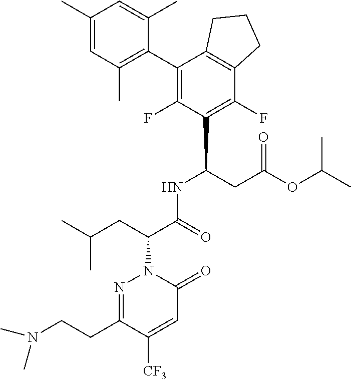 [SMILES: Cc1cc(C)c(-c2c(F)c([C@@H](CC(=O)OC(C)C)NC(=O)[C@@H](CC(C)C)n3nc(CCN(C)C)c(C(F)(F)F)cc3=O)c(F)c3c2CCC3)c(C)c1] |
| 446 | 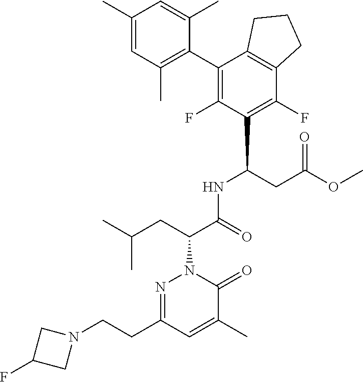 [SMILES: COC(=O)C[C@@H](NC(=O)[C@@H](CC(C)C)n1nc(CCN2CC(F)C2)cc(C)c1=O)c1c(F)c2c(c(-c3c(C)cc(C)cc3C)c1F)CCC2] |
| 447 | 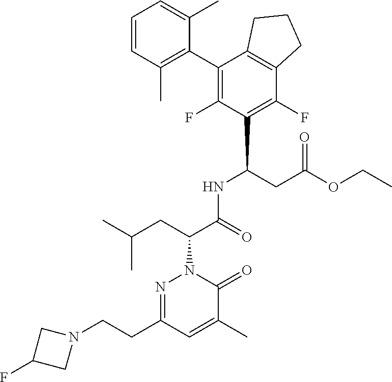 [SMILES: CCOC(=O)C[C@@H](NC(=O)[C@@H](CC(C)C)n1nc(CCN2CC(F)C2)cc(C)c1=O)c1c(F)c2c(c(-c3c(C)cccc3C)c1F)CCC2] |
| 448 | 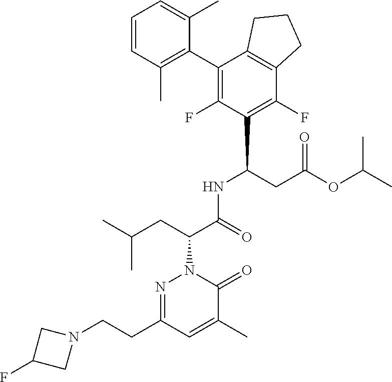 [SMILES: Cc1cccc(C)c1-c1c(F)c([C@@H](CC(=O)OC(C)C)NC(=O)[C@@H](CC(C)C)n2nc(CCN3CC(F)C3)cc(C)c2=O)c(F)c2c1CCC2] |
| 449 | 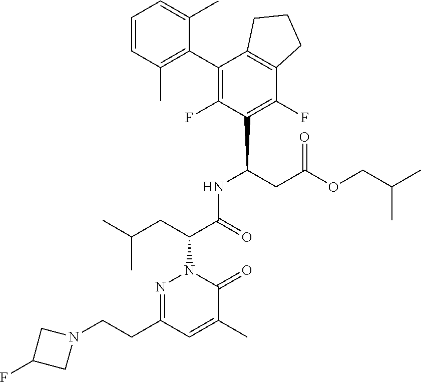 [SMILES: Cc1cccc(C)c1-c1c(F)c([C@@H](CC(=O)OCC(C)C)NC(=O)[C@@H](CC(C)C)n2nc(CCN3CC(F)C3)cc(C)c2=O)c(F)c2c1CCC2] |
| 450 | 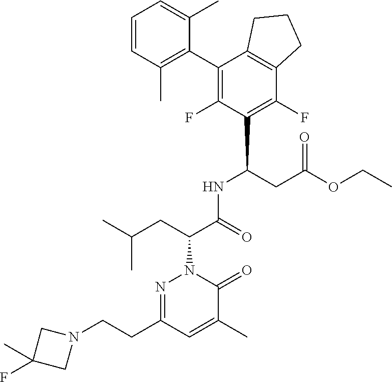 [SMILES: CCOC(=O)C[C@@H](NC(=O)[C@@H](CC(C)C)n1nc(CCN2CC(C)(F)C2)cc(C)c1=O)c1c(F)c2c(c(-c3c(C)cccc3C)c1F)CCC2] |
| 451 | 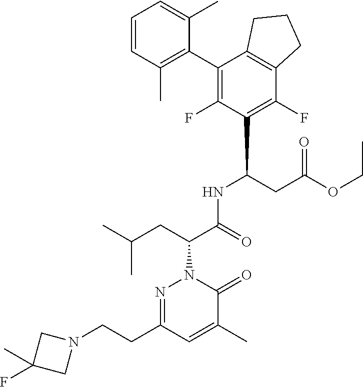 [SMILES: CCOC(=O)C[C@@H](NC(=O)[C@@H](CC(C)C)n1nc(CCN2CC(C)(F)C2)cc(C)c1=O)c1c(F)c2c(c(-c3c(C)cccc3C)c1F)CCC2] |
| 452 | 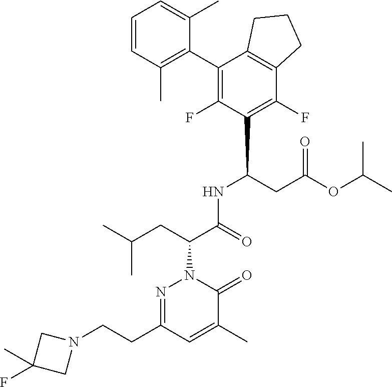 [SMILES: Cc1cccc(C)c1-c1c(F)c([C@@H](CC(=O)OC(C)C)NC(=O)[C@@H](CC(C)C)n2nc(CCN3CC(C)(F)C3)cc(C)c2=O)c(F)c2c1CCC2] |
| 453 | 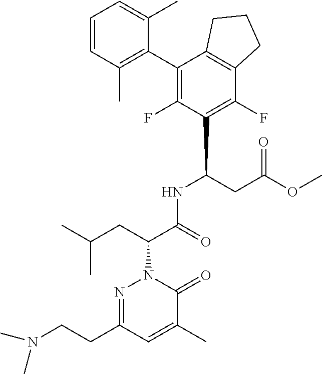 |
| 454 | 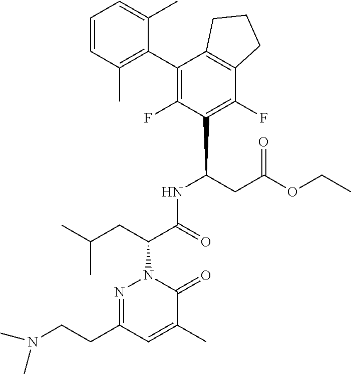 [SMILES: CCOC(=O)C[C@@H](NC(=O)[C@@H](CC(C)C)n1nc(CCN(C)C)cc(C)c1=O)c1c(F)c2c(c(-c3c(C)cccc3C)c1F)CCC2] |
| 455 | 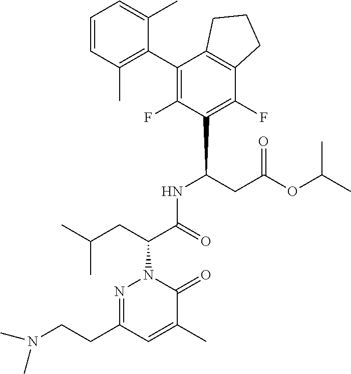 [SMILES: Cc1cccc(C)c1-c1c(F)c([C@@H](CC(=O)OC(C)C)NC(=O)[C@@H](CC(C)C)n2nc(CCN(C)C)cc(C)c2=O)c(F)c2c1CCC2] |
| 456 | 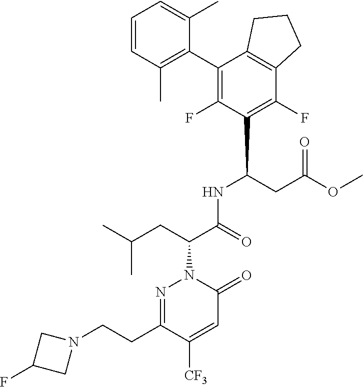 [SMILES: COC(=O)C[C@@H](NC(=O)[C@@H](CC(C)C)n1nc(CCN2CC(F)C2)c(C(F)(F)F)cc1=O)c1c(F)c2c(c(-c3c(C)cccc3C)c1F)CCC2] |
| 457 | 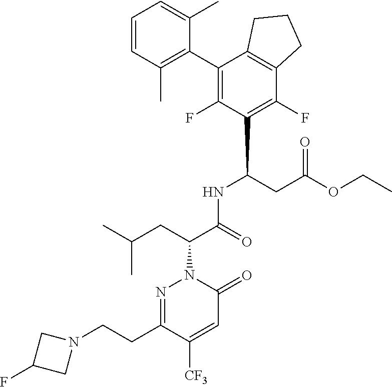 [SMILES: CCOC(=O)C[C@@H](NC(=O)[C@@H](CC(C)C)n1nc(CCN2CC(F)C2)c(C(F)(F)F)cc1=O)c1c(F)c2c(c(-c3c(C)cccc3C)c1F)CCC2] |
| 458 | 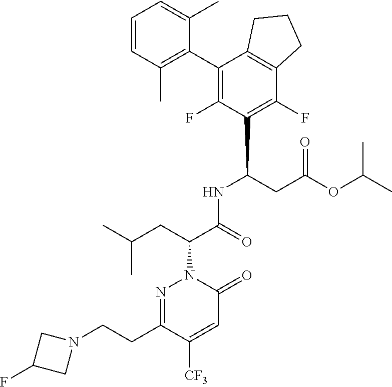 [SMILES: Cc1cccc(C)c1-c1c(F)c([C@@H](CC(=O)OC(C)C)NC(=O)[C@@H](CC(C)C)n2nc(CCN3CC(F)C3)c(C(F)(F)F)cc2=O)c(F)c2c1CCC2] |
| 459 | 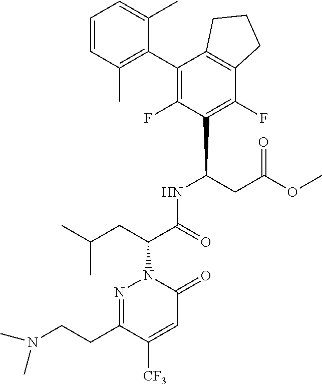 |
| 460 | 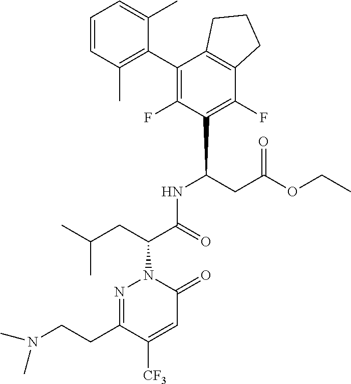 [SMILES: CCOC(=O)C[C@@H](NC(=O)[C@@H](CC(C)C)n1nc(CCN(C)C)c(C(F)(F)F)cc1=O)c1c(F)c2c(c(-c3c(C)cccc3C)c1F)CCC2] |
| 461 | 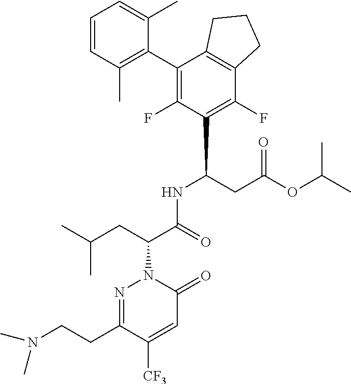 [SMILES: Cc1cccc(C)c1-c1c(F)c([C@@H](CC(=O)OC(C)C)NC(=O)[C@@H](CC(C)C)n2nc(CCN(C)C)c(C(F)(F)F)cc2=O)c(F)c2c1CCC2] |

The present disclosure relates to a pharmaceutical composition, comprising the compound or the stereoisomer, mesomer/racemate, deuterated product, solvate, prodrug, metabolite, pharmaceutically acceptable salt, or co-crystal thereof according to the present disclosure, and a pharmaceutically acceptable carrier.

The present disclosure relates to the use of the compound or the stereoisomer, mesomer/racemate, deuterated product, solvate, prodrug, metabolite, pharmaceutically acceptable salt, or co-crystal thereof according to the present disclosure in the preparation of a drug for the treatment of diseases related to α4β7 activity or expression, preferably the use in the preparation of a drug for inflammatory bowel diseases.

The present disclosure relates to a pharmaceutical composition or pharmaceutical preparation comprising a therapeutically effective amount of the compound or the stereoisomer, mesomer/racemate, deuterated product, solvate, prodrug, metabolite, pharmaceutically acceptable salt, or co-crystal thereof according to the present disclosure, and a pharmaceutical excipient. The pharmaceutical composition can be in a unit preparation form (the amount of the active drug in the unit preparation is also referred to as “preparation specification”).

The present disclosure further provides a method for treating a disease in a mammal, comprising administering to the mammal a therapeutically effective amount of the compound or the stereoisomer, mesomer/racemate, deuterated product, solvate, prodrug, metabolite, pharmaceutically acceptable salt, or co-crystal thereof or the pharmaceutical composition according to the present disclosure. In some embodiments, the mammal mentioned in the present disclosure includes human.

The term “effective amount” or “therapeutically effective amount” in the present application refers to a sufficient amount of the compound disclosed in the present application being administered which will relieve to some extent one or more of the symptoms of the disease or condition being treated, e.g., inflammatory bowel diseases. In some embodiments, the outcome is the reduction and/or remission of signs, symptoms, or causes of the disease, or any other desired change in the biological system. For example, an “effective amount” in terms of the therapeutic use is an amount comprising the compound disclosed in the present application that is required to provide clinically significant reduction of the symptoms of the disease. Examples of the therapeutically effective amount includes, but are not limited to 1-1500 mg, 1-1200 mg, 1-1000 mg, 1-900 mg, 1-800 mg, 1-700 mg, 1-600 mg, 2-600 mg, 3-600 mg, 4-600 mg, 5-600 mg, 6-600 mg, 10-600 mg, 20-600 mg, 25-600 mg, 30-600 mg, 40-600 mg, 50-600 mg, 60-600 mg, 70-600 mg, 75-600 mg, 80-600 mg, 90-600 mg, 100-600 mg, 200-600 mg, 1-500 mg, 2-500 mg, 3-500 mg, 4-500 mg, 5-500 mg, 6-500 mg, 10-500 mg, 20-500 mg, 25-500 mg, 30-500 mg, 40-500 mg, 50-500 mg, 60-500 mg, 70-500 mg, 75-500 mg, 80-500 mg, 90-500 mg, 100-500 mg, 125-500 mg, 150-500 mg, 200-500 mg, 250-500 mg, 300-500 mg, 400-500 mg, 5-400 mg, 10-400 mg, 20-400 mg, 25-400 mg, 30-400 mg, 40-400 mg, 50-400 mg, 60-400 mg, 70-400 mg, 75-400 mg, 80-400 mg, 90-400 mg, 100-400 mg, 125-400 mg, 150-400 mg, 200-400 mg, 250-400 mg, 300-400 mg, 1-300 mg, 2-300 mg, 5-300 mg, 10-300 mg, 20-300 mg, 25-300 mg, 30-300 mg, 40-300 mg, 50-300 mg, 60-300 mg, 70-300 mg, 75-300 mg, 80-300 mg, 90-300 mg, 100-300 mg, 125-300 mg, 150-300 mg, 200-300 mg, 250-300 mg, 1-200 mg, 2-200 mg, 5-200 mg, 10-200 mg, 20-200 mg, 25-200 mg, 30-200 mg, 40-200 mg, 50-200 mg, 60-200 mg, 70-200 mg, 75-200 mg, 80-200 mg, 90-200 mg, 100-200 mg, 125-200 mg, 150-200 mg, 80-1000 mg, or 80-800 mg.

In some embodiments, the pharmaceutical composition comprises the compound or the stereoisomer, mesomer/racemate, deuterated product, solvate, prodrug, metabolite, pharmaceutically acceptable salt, or co-crystal thereof according to the present disclosure in an amount including but not limited to 1-1000 mg, 20-800 mg, 40-800 mg, 40-400 mg, 25-200 mg, 1 mg, 5 mg, 10 mg, 15 mg, 20 mg, 25 mg, 30 mg, 35 mg, 40 mg, 45 mg, 50 mg, 55 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, 300 mg, 320 mg, 400 mg, 480 mg, 500 mg, 600 mg, 640 mg and 840 mg.

The present disclosure relates to a method for treating a disease in a mammal, the method comprising administering to a subject a therapeutically effective amount of the compound or the stereoisomer, mesomer/racemate, deuterated product, solvate, prodrug, metabolite, pharmaceutically acceptable salt, or co-crystal thereof according to the present disclosure, wherein the therapeutically effective amount is preferably 1-1500 mg, and the disease is preferably inflammatory bowel disease.

The present disclosure relates to a method for treating a disease in a mammal, the method comprising administering to a subject a drug, i.e., the compound or the stereoisomer, mesomer/racemate, deuterated product, solvate, prodrug, metabolite, pharmaceutically acceptable salt, or co-crystal thereof according to the present disclosure in a daily dose of 1-1000 mg/day, wherein the daily dose can be a single dose or divided doses; in some embodiments, the daily dose includes, but is not limited to 10-1500 mg/day, 10-1000 mg/day, 10-800 mg/day, 25-800 mg/day, 50-800 mg/day, 100-800 mg/day, 200-800 mg/day, 25-400 mg/day, 50-400 mg/day, 100-400 mg/day, or 200-400 mg/day; in some embodiments, the daily dose includes, but is not limited to 10 mg/day, 20 mg/day, 25 mg/day, 50 mg/day, 80 mg/day, 100 mg/day, 125 mg/day, 150 mg/day, 160 mg/day, 200 mg/day, 300 mg/day, 320 mg/day, 400 mg/day, 480 mg/day, 600 mg/day, 640 mg/day, 800 mg/day, or 1000 mg/day.

The present disclosure relates to a kit, wherein the kit may comprise a composition in the form of a single dose or multiple doses and comprises the compound or the stereoisomer, mesomer/racemate, deuterated product, solvate, prodrug, metabolite, pharmaceutically acceptable salt, or co-crystal thereof according to the present disclosure, and the amount of the compound or the stereoisomer, mesomer/racemate, deuterated product, solvate, prodrug, metabolite, pharmaceutically acceptable salt, or co-crystal thereof according to the present disclosure is identical to the amount of same in the above-mentioned pharmaceutical composition.

In the present disclosure, the amount of the compound or the stereoisomer, mesomer/racemate, deuterated product, solvate, prodrug, metabolite, pharmaceutically acceptable salt, or co-crystal thereof according to the present disclosure is calculated in the form of free base in each case.

The term “preparation specification” refers to the weight of the active drug contained in each vial, tablet, or other unit preparation.

Synthetic method of the compound according to the present disclosure:

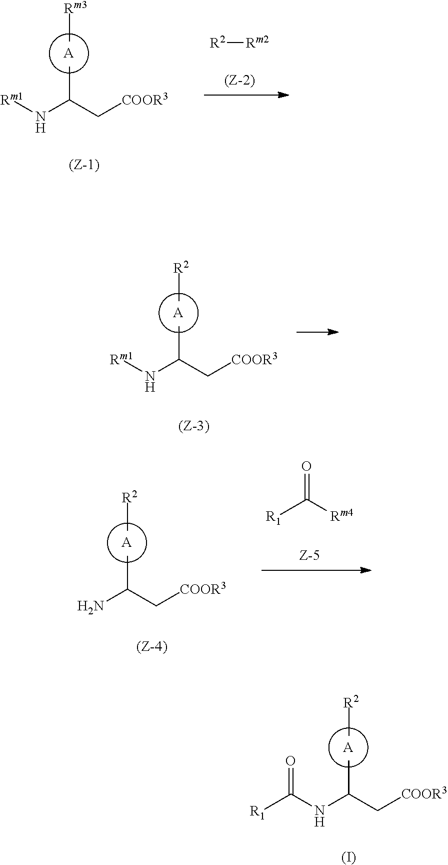

a compound of general formula (Z-1) and a compound of general formula (Z-2) are subjected to coupling or substitution reaction to obtain a compound of general formula (Z-3);
the protecting group of the compound of general formula (Z-3) is removed to obtain a compound of general formula (Z-4); and
the compound of general formula (Z-4) and a compound of general formula (Z-5) are subjected to coupling or substitution reaction to obtain the compound of general formula (I), wherein
R^{m1} is selected from Boc; Cbz; tert-butylsulfinyl, etc.;
R^{m2} and R^{m3} are each independently selected from H, Cl, Br, I, OTf, B(OH)₂, boronic ester group, alkyl-substituted stannyl, etc.;
R^{m4} is selected from Cl, Br, I, OH, etc.; and
the definitions of the remaining groups are the same as those in any one of the embodiments of the above general formula (I).

Unless otherwise stated, the terms used in the description and the claims have the following meanings.

The carbon, hydrogen, oxygen, sulfur, nitrogen or F, Cl, Br, I involved in the groups and compounds of the present disclosure all comprise their isotopes, and the carbon, hydrogen, oxygen, sulfur or nitrogen involved in the groups and compounds of the present disclosure is optionally replaced with one or more of their corresponding isotopes, wherein the isotopes of carbon comprise ¹²C, ¹³C and ¹⁴C, the isotopes of hydrogen comprise protium (H), deuterium (D, also known as heavy hydrogen), tritium (T, also known as superheavy hydrogen), the isotopes of oxygen comprise ¹⁶O, ¹⁷O and ¹⁸O, the isotopes of sulfur comprise ³²S, ³³S, ³⁴S and ³⁶S, the isotopes of nitrogen comprise ¹⁴N and ¹⁵N, the isotopes of fluorine comprise ¹⁷F and ¹⁹F, the isotopes of chlorine comprise ³⁵Cl and ³⁷Cl, and the isotopes of bromine comprise ⁷⁹Br and ⁸¹Br.

“Alkyl” refers to a linear or branched saturated aliphatic hydrocarbon group containing 1 to 20 carbon atoms, preferably alkyl containing 1 to 8 carbon atoms, more preferably alkyl containing 1 to 6 carbon atoms, further preferably alkyl containing 1 to 4 carbon atoms. Non-limiting examples include methyl, ethyl, n-propyl, isopropyl, n-butyl, sec-butyl, neobutyl, tert-butyl, n-pentyl, isoamyl, neopentyl, n-hexyl and various branched isomers thereof. The alkyl is optionally substituted with 0 to 6 substituents selected from F, Cl, Br, I, hydroxyl, sulfhydryl, nitro, cyano, amino, alkylamino, amido, alkenyl, alkynyl, C₁₋₆ alkyl, C₁₋₆ hydroxyalkyl, C₁₋₆ alkoxy, 3- to 8-membered carbocyclyl, 3- to 8-membered heterocyclyl, 3- to 8-membered carbocyclyloxy, 3- to 8-membered heterocyclyloxy, carboxyl or a carboxylate group.

“Alkylene” refers to linear and branched divalent saturated hydrocarbon groups, including —(CH₂)ᵥ— (v is an integer from 1 to 10), and examples of alkylene include, but are not limited to, methylene, ethylene, propylene, butylene, etc. The alkylene is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group.

“Cycloalkyl” refers to a monovalent saturated carbocyclic hydrocarbon group, usually having from 3 to 12 carbon atoms, and including monocyclic cycloalkyl, fused cycloalkyl, spiro cycloalkyl or bridged cycloalkyl, and non-limiting examples include cyclopropyl, cyclobutyl, cyclopentyl, cyclohexyl, cycloheptyl, etc. The cycloalkyl is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group.

“Alkenyl” refers to linear and branched monovalent unsaturated hydrocarbon groups having at least 1, usually 1, 2 or 3 carbon-carbon double bonds, in which the main chain comprises 2 to 10 carbon atoms, further preferably 2 to 6 carbon atoms, and more preferably 2 to 4 carbon atoms. Examples of alkenyl include, but are not limited to ethenyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene and 1,4-hexadiene. The alkenyl is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group.

“Alkynyl” refers to linear and branched unsaturated hydrocarbon groups having at least 1, usually 1, 2 or 3 carbon-carbon triple bonds, in which the main chain comprises 2 to 10 carbon atoms, further preferably 2 to 6 carbon atoms, and more preferably 2 to 4 carbon atoms. Examples of alkynyl include but are not limited to ethynyl, propargyl, 1-propynyl, 2-propynyl, 1-butynyl, 2-butynyl, 3-butynyl, 1-pentynyl, 2-pentynyl, 3-pentynyl, 4-pentynyl, 1-methyl-1-butynyl, 2-methyl-1-butynyl, 2-methyl-3-butynyl, 1-hexynyl, 2-hexynyl, 3-hexynyl, 4-hexynyl, 5-hexynyl, 1-methyl-1-pentynyl, 2-methyl-1-pentynyl, 1-heptynyl, 2-heptynyl, 3-heptynyl, 4-heptynyl, 1-octynyl, 3-octynyl, 1-nonynyl, 3-nonynyl, 1-decynyl and 4-decynyl. The alkynyl is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group. The definition of the alkynyl described herein is consistent with this definition.

“Alkoxy” refers to —O-alkyl. Non-limiting examples include methoxy, ethoxy, n-propoxy, isopropoxy, n-butoxy, sec-butoxy, tert-butoxy, n-pentoxy, n-hexyloxy, cyclopropoxy and cyclobutoxy. The alkoxy is optionally substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group.

“Carbocyclyl” or “carbocyclic ring” refers to a substituted or unsubstituted saturated or unsaturated aromatic ring or non-aromatic ring, wherein the aromatic ring or non-aromatic ring can be a 3- to 8-membered monocyclic ring, a 4- to 12-membered bicyclic ring or a 10- to 15-membered tricyclic ring system. Carbocyclyl can be connected to an aromatic ring or a non-aromatic ring, wherein the aromatic ring or non-aromatic ring is optionally a monocyclic ring, a bridged ring or a spiro ring. Non-limiting examples include cyclopropane, cyclobutane, cyclopentane, cyclohexane, cycloheptane, 1-cyclopentyl-1-enyl, 1-cyclopentyl-2-enyl, 1-cyclopentyl-3-enyl, cyclohexyl, 1-cyclohexyl-2-enyl, 1-cyclohexyl-3-enyl, cyclohexenyl, benzene ring, naphthalene ring,

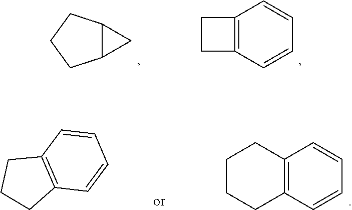 [SMILES: C1CC2CC2C1 ; c1ccc2c(c1)CC2 ; c1ccc2c(c1)CCC2 ; c1ccc2c(c1)CCCC2]

The carbocyclic ring is optionally substituted with 0 to 5 substituents selected from F, Cl, Br, I, ═O, hydroxyl, sulfhydryl, nitro, cyano, amino, alkylamino, amido, alkenyl, alkynyl, alkyl, hydroxyalkyl, alkoxy, carbocyclyl, heterocyclyl, carbocyclyloxy, heterocyclyloxy, carboxyl or a carboxylate group.

“Heterocyclyl” or “heterocyclic ring” refers to a substituted or unsubstituted saturated or unsaturated aromatic ring or non-aromatic ring, and the aromatic ring or non-aromatic ring can be a 3- to 8-membered monocyclic ring, a 4- to 12-membered bicyclic ring or a 10- to 15-membered tricyclic ring system, and contains 1 to 3 heteroatoms selected from N, O or S, preferably 3- to 8-membered heterocyclyl, and the optionally substituted N, S in the ring of heterocyclyl can be oxidized into various oxidation states. Heterocyclyl can be connected to a heteroatom or a carbon atom; heterocyclyl can be connected to an aromatic ring or a non-aromatic ring; and heterocyclyl can be connected to a bridged ring or a spiro ring. Non-limiting examples include oxiranyl, aziridinyl, oxetanyl, azetidinyl, 1,3-dioxolanyl, 1,4-dioxolanyl, 1,3-dioxanyl, azepanyl, pyridyl, furyl, thienyl, pyranyl, N-alkylpyrrolyl, pyrimidinyl, pyrazinyl, pyridazinyl, imidazolyl, piperidyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, tetrahydrothiazolyl, tetrahydropyranyl, benzoimidazolyl, benzopyridyl, pyrrolopyridyl, benzodihydrofuryl, pyrrolyl, pyrazolyl, thiazolyl, oxazolyl, pyrazinyl, indazolyl, benzothienyl, benzofuryl, benzopyrrolyl, benzoimidazolyl, benzothiazolyl, benzoxazolyl, benzopyridyl, benzopyrimidinyl, benzopyrazinyl, piperazinyl, azabicyclo[3.2.1]octanyl, azabicyclo[5.2.0]nonanyl, oxatricyclo[5.3.1.1]dodecyl, azaadamantyl, oxaspiro[3.3]heptanyl,

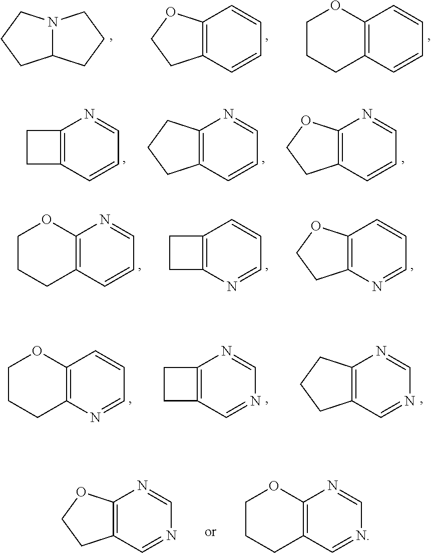

The heterocyclyl is optionally substituted with 0 to 5 substituents selected from F, Cl, Br, I, ═O, hydroxyl, sulfhydryl, nitro, cyano, amino, alkylamino, amido, alkenyl, alkynyl, alkyl, hydroxyalkyl, alkoxy, carbocyclyl, heterocyclyl, carbocyclyloxy, heterocyclyloxy, carboxyl or a carboxylate group.

“Heterocycloalkyl” refers to substituted or unsubstituted saturated heterocyclyl, and can be a 3- to 8-membered monocyclic ring, a 4- to 12-membered bicyclic ring or a 10- to 15-membered tricyclic ring system, and contains 1 to 3 heteroatoms selected from N, O or S, preferably 3- to 8-membered heterocyclyl, and the optionally substituted N, S in the ring of heterocycloalkyl can be oxidized into various oxidation states. Heterocycloalkyl can be connected to a heteroatom or a carbon atom. Non-limiting examples include oxiranyl, aziridinyl, oxetanyl, azetidinyl, 1,3-dioxolanyl, 1,4-dioxolanyl, 1,3-dioxanyl, azepanyl, piperidyl, or morpholinyl. The heterocycloalkyl is optionally substituted with 0 to 5 substituents selected from F, Cl, Br, I, ═O, hydroxyl, sulfhydryl, nitro, cyano, amino, alkylamino, amido, alkenyl, alkynyl, alkyl, hydroxyalkyl, alkoxy, carbocyclyl, heterocyclyl, carbocyclyloxy, heterocyclyloxy, carboxyl or a carboxylate group.

“Spiro ring” refers to a 5- to 20-membered polycyclic group sharing one atom (referred to as a spiro atom) between substituted or unsubstituted monocyclic rings, which may contain 0 to 5 double bonds, and may contain 0 to 5 heteroatoms selected from N, O or S(═O)n (n is selected from 0, 1 or 2). The spiro ring is preferably 6- to 14-membered, further preferably 6- to 12-membered, and more preferably 6- to 10-membered. Its non-limiting examples include:

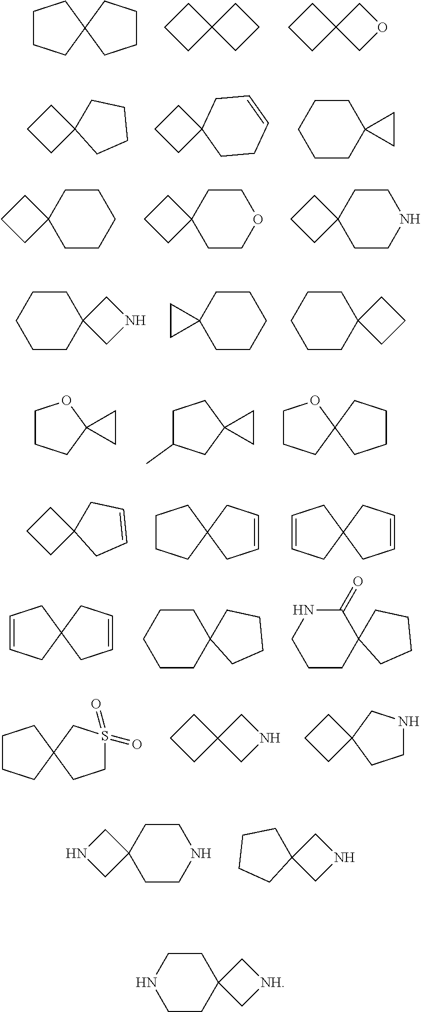

“Fused ring” refers to a polycyclic group in which each ring in the system shares an adjacent pair of atoms with other rings in the system, wherein one or more of the rings may contain 0 or more double bonds, which may be substituted or unsubstituted, and each ring in the fused ring system may contain 0 to 5 heteroatoms selected from N, S(═O)n or O (n is selected from 0, 1 or 2). The fused ring is preferably 5- to 20-membered, further preferably 5- to 14-membered, more preferably 5- to 12-membered, and still further preferably 5- to 10-membered. Non-limiting examples include:

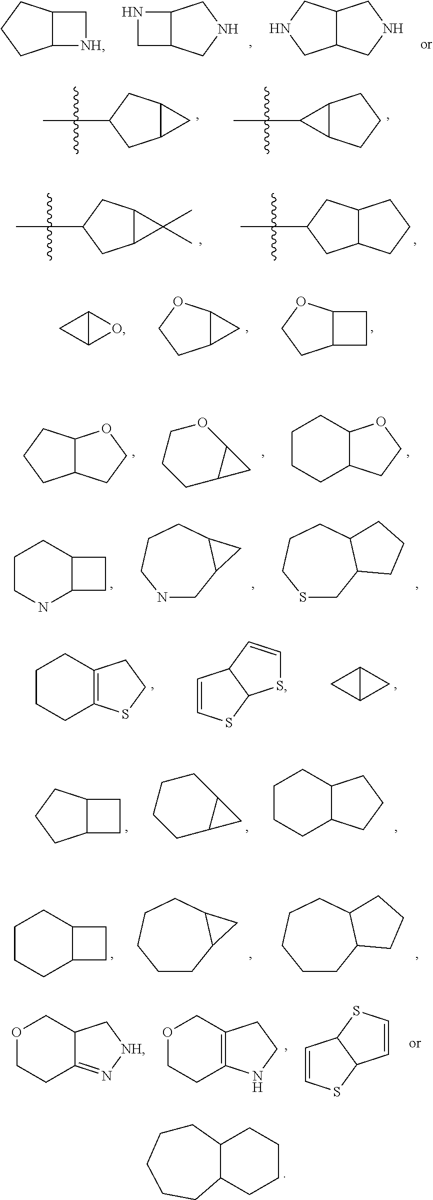

“Bridged ring” refers to a polycyclic group containing any two atoms that are not directly connected, which group may contain 0 or more double bonds and can be substituted or unsubstituted, and any ring in the bridged ring system may contain 0 to 5 heteroatoms or groups selected from N, S(═O)n or O (wherein n is 0, 1 or 2). The ring atoms contain 5 to 20 atoms, preferably 5 to 14 atoms, further preferably 5 to 12 atoms, and still further preferably 5 to 10 atoms. Non-limiting examples include

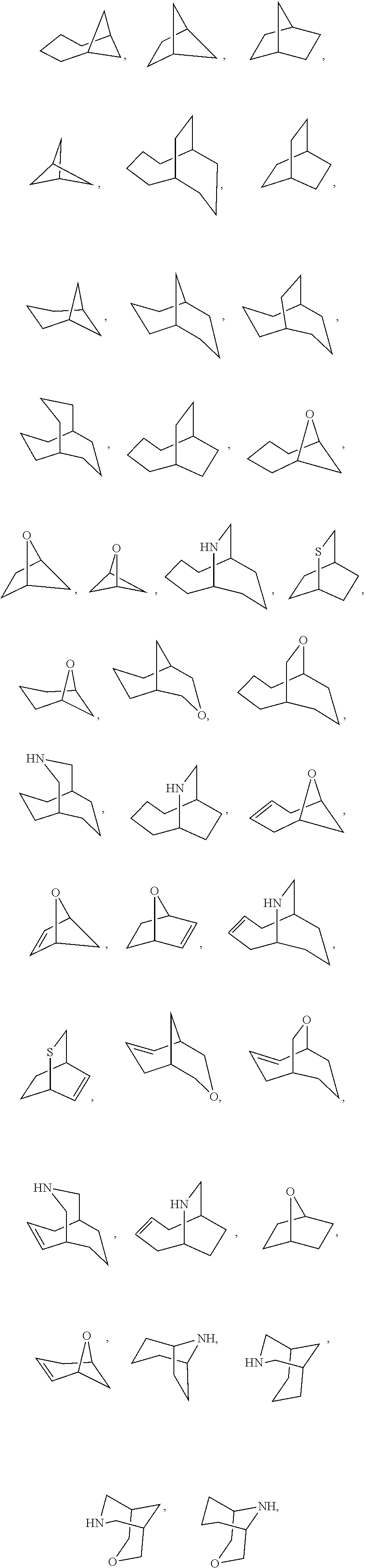

and adamantane. The definition of the bridged ring described herein is consistent with this definition.

“Carbospiro ring”, “spiro ring carbocyclyl”, “spirocarbocyclyl” or “carbospiro ring group” refers to a “spiro ring” with a ring system consisting only of carbon atoms.

“Carbo-fused ring”, “fused ring carbocyclyl”, “fused carbocyclyl” or “carbo-fused ring group” refers to a “fused ring” with a ring system consisting only of carbon atoms.

“Carbo-bridged ring”, “bridged ring carbocyclyl”, “bridged carbocyclyl” or “carbo-bridged ring group” refers to a “bridged ring” with a ring system consisting only of carbon atoms.

“Heteromonocyclic ring”, “monocyclic heterocyclyl” or “heteromonocyclyl” means “heterocyclyl” or “heterocyclic ring” with a monocyclic system.

“Fused-heterocyclic ring”, “fused-heterocyclyl”, “fused ring heterocyclyl” or “fused-heterocyclic ring group” refers to a “fused ring” containing a heteroatom.

“Spiro-heterocyclic ring”, “spiro-heterocyclyl”, “spiro ring heterocyclyl” or “spiro-heterocyclic ring group” refers to a “spiro ring” containing a heteroatom.

“Bridged-heterocyclic ring”, “bridged-heterocyclyl” or “bridged ring heterocyclyl” refers to a “bridged ring” containing a heteroatom.

“Aryl” or “aromatic ring” refers to a monovalent aromatic hydrocarbon group with a monocyclic ring or a fused ring, which generally has 6 to 12 carbon atoms and can be substituted or unsubstituted. The definition of the aryl or aromatic ring described herein is consistent with this definition.

“Heteroaryl” refers to substituted or unsubstituted 5- to 15-membered aromatic ring, contains 1 to 5 heteroatoms or groups selected from N, O or S(═O)n, and is preferably 5- to 10-membered heteraromatic ring, and further preferably 5- to 6-membered heteraromatic ring. Non-limiting examples of heteroaryl include, but are not limited to pyridyl, furyl, thienyl, pyridinyl, pyranyl, N-alkylpyrrolyl, pyrimidinyl, pyrazinyl, pyridazinyl, imidazolyl, benzopyrazole, benzimidazolyl, benzopyridine, pyrrolopyridine, etc. The heteroaryl ring can be fused to an aryl, heterocyclyl or cycloalkyl ring, wherein the ring connected to the parent structure is a heteroaryl ring, and non-limiting examples include

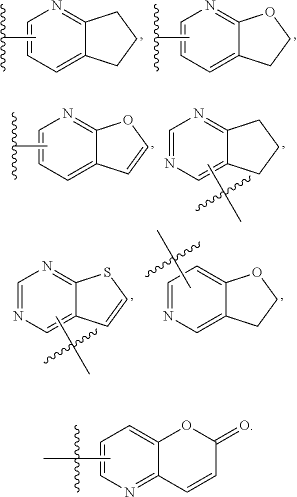

“Containing 1 to 4 heteroatoms selected from O, S or N” refers to contains 1, 2, 3 or 4 heteroatoms selected from O, S or N.

“Substitution” or “substituted” refers to substitution with 1 or more (including but not limited to 2, 3, 4 or 5) substituents including but not limited to H, F, Cl, Br, I, alkyl, cycloalkyl, alkoxy, haloalkyl, mercaptan, hydroxyl, nitro, mercapto, amino, cyano, isocyano, aryl, heteroaryl, heterocyclyl, bridged ring group, spiro ring group, fused ring group, hydroxyalkyl, ═O, carbonyl, aldehyde, carboxylic acid, carboxylate, —(CH₂)ₘ—C(═O)—R^{a}, —O—(CH₂)ₘ—C(═O)—R^{a}, —(CH₂)ₘ—C(═O)—NR^{b}R^{c}, —(CH₂)ₘS(═O)ₙR^{a}, —(CH₂)ₘ-alkenyl-R^{a}, OR^{d} or —(CH₂)ₘ-alkynyl-R^{a} (wherein m and n are 0, 1 or 2), arylthio, thiocarbonyl, silyl or —NR^{b}R^{c} and the like, wherein R^{b} and R^{c} are independently selected from H, hydroxyl, amino, carbonyl, alkyl, alkoxy, cycloalkyl, heterocyclyl, aryl, heteroaryl, sulfonyl, or trifluoromethylsulfonyl. As an option, R^{b} and R^{c} may form a five- or six-membered cycloalkyl or heterocyclyl; R^{a} and R^{d} are each independently selected from aryl, heteroaryl, alkyl, alkoxy, cycloalkyl, heterocyclyl, carbonyl, ester group, bridged ring group, spiro ring group or fused ring group.

“Substituted with 0 to X substituents” refers to substituted with 0, 1, 2, 3 . . . X substituents, wherein X is selected from any integer between 1 and 10. For example, “substituted with 0 to 4 substituents” refers to substituted with 0, 1, 2, 3 or 4 substituents. For example, “substituted with 0 to 5 substituents” refers to substituted with 0, 1, 2, 3, 4 or 5 substituents. For example, “bridged-heterocyclic ring is optionally substituted with 0 to 4 substituents selected from H or F” means that the bridged-heterocyclic ring is optionally substituted with 0, 1, 2, 3 or 4 substituents selected from H or F.

An X- to Y-membered ring (X is selected from an integer less than Y and greater than or equal to 3, and Y is selected from any integer between 4 and 12) includes X-, X+1-, X+2-, X+3-, X+4-, . . . , to Y-membered rings. Rings include heterocyclic ring, carbocyclic ring, aromatic ring, aryl, heteroaryl, cycloalkyl, mono-heterocyclic ring, fused-heterocyclic ring, spiro-heterocyclic ring or bridged-heterocyclic ring. For example, a “4- to 7-membered mono-heterocyclic ring” refers to a 4-, 5-, 6- or 7-membered mono-heterocyclic ring, and a “5- to 10-membered fused-heterocyclic ring” refers to a 5-, 6-, 7-, 8-, 9- or 10-membered fused-heterocyclic ring.

The term “optional” or “optionally” means that the events or circumstances described subsequently may but not necessarily occur, and the description includes the occasions where the events or circumstances occur or do not occur. For example, “alkyl optionally substituted with F” means that the alkyl may but not necessarily be substituted with F, and the description includes the case where the alkyl is substituted with F and the case where the alkyl is not substituted with F.

“Pharmaceutically acceptable salt” or “pharmaceutically acceptable salt thereof” refers to a salt of the compound according to the present disclosure, which salt maintains the biological effectiveness and characteristics of a free acid or a free base, and is obtained by reacting the free acid with a non-toxic inorganic base or organic base, or reacting the free base with a non-toxic inorganic acid or organic acid.

“Pharmaceutical composition” refers to a mixture of one or more of the compounds of the present disclosure, a pharmaceutically acceptable salt or a prodrug thereof, and other chemical components, wherein “other chemical components” refer to pharmaceutically acceptable carriers, excipients and/or one or more other therapeutic agents.

“Carrier” refers to a material that does not cause significant irritation to an organism and does not eliminate the biological activity and characteristics of a compound administered.

“Animal” refers to include mammals, such as humans, companion animals, zoo animals, and domestic animals, preferably humans, horses, or dogs.

The term “stereoisomer” refers to an isomer produced as a result of different spatial arrangement of atoms in molecules, including cis-trans isomers, enantiomers and conformational isomers.

### DETAILED DESCRIPTION

To achieve the objectives of the present disclosure, according to organic synthesis techniques known to those skilled in the art, and starting from commercially available chemicals and/or compounds described in chemical documents, the prepared compounds, “commercially available chemicals”, for use in the reactions described herein are obtained from standard commercial sources, including Shanghai Aladdin Bio-Chem Technology Co., Ltd., Shanghai Macklin Biochemical Co., Ltd., Sigma-Aldrich, Alfa Aesar (China) Chemical Co., Ltd., Tokyo Chemical Industry (Shanghai) Co., Ltd., Energy Chemical Co., Ltd., Shanghai Titan Scientific Co., Ltd., Kelong Chemical Co., Ltd., J&K Scientific and the like.

The technical solutions of the present disclosure will be described in detail by the following examples, but the scope of protection of the present disclosure includes but is not limited thereto.

The structures of the compounds are determined by nuclear magnetic resonance (NMR) or (and) mass spectrometry (MS). The NMR shift (δ) is given in the unit of 10-6 (ppm). NMR is determined with Bruker Avance III 400 and Bruker Avance 300; the solvent for determination is deuterated dimethyl sulfoxide (DMSO-d6), deuterated chloroform (CDCl3) and deuterated methanol (CD3OD); and the internal standard is tetramethylsilane (TMS);
MS is determined with Agilent 6120B (ESI) and Agilent 6120B (APCI); and
HPLC is determined with Agilent 1260DAD high pressure liquid chromatograph (Zorbax SB-C18 100×4.6 mm, 3.5 μM);
Yantai Huanghai HSGF254 or Qingdao GF254 silica gel plate is used as a thin layer chromatography silica plate, and the silica gel plate for the thin layer chromatography (TLC) is of the specification of 0.15 mm-0.20 mm, and the specification when separating and purifying a product by thin layer chromatography is 0.4 mm-0.5 mm;
and for the column chromatography, Yantai Huanghai silica gel of 200-300 mesh silica gel is generally used as a carrier.

Boc: tert-butoxycarbonyl; Ts: p-toluenesulfonyl; Cbz: benzyloxycarbonyl; TMS: trimethylsilyl;
TIPS: triisopropylsilyl; Bpin: pinacol boronate; MOM: methylenemethyl ether; THF: Tetrahydrofuran
HATU: 2-(7-azabenzotriazole)-N,N,N′,N′-tetramethyluronium hexafluorophosphate;
PdCl2(dppf): CAS 72287-26-4; XPhos Pd G2: CAS 1310584-14-5
* next to a chemical bond represents that the chirality of the chiral atom is R or S;
Rt, retention time: the retention time of the analytical method, not specifically stated in the examples, is as follows:
Detection wavelength: 254 nm/210 nm Flow rate: 1.0 ml/min Column temperature: 35° C.
Injection volume: 2 μl Acquisition time: 10 min
Gradient elution procedure:

| Time (min) | Mobile phase A (%) | Mobile phase B (%) |
|---|---|---|
| 0 | 95 | 5 |
| 5 | 5 | 95 |
| 7 | 5 | 95 |
| 7.01 | 95 | 5 |
| 10 | 95 | 5 |

Acidic conditions: Mobile phase A: 0.05% TFA solution, mobile phase B: acetonitrile Chromatographic column brand: Welch, chromatographic column model: Xtimate C18 4.6*50 mm, 3 μm
Instrument model 1: Shimadzu LC-20AT, Instrument code: CH—Y-J0460
Instrument model 2: Agilent 1260 Infinity, Instrument code: CH—Y-J0692

The specific method for preparative HPLC (prep-HPLC) is as follows: Method 1: instrument: waters 2767 preparative chromatographic column: SunFire® Prep C18 (19 mm×150 mm); composition of mobile phases: mobile phase A: acetonitrile, and mobile phase B: water (containing 0.1% ammonium acetate). Method 2: instrument: waters 2767 preparative chromatographic column: SunFire® Prep C18 (19 mm×150 mm); composition of mobile phases: mobile phase A: acetonitrile, and mobile phase B: water (containing 0.1% trifluoroacetic acid).

### Intermediate 1: Preparation of Intermediate 1

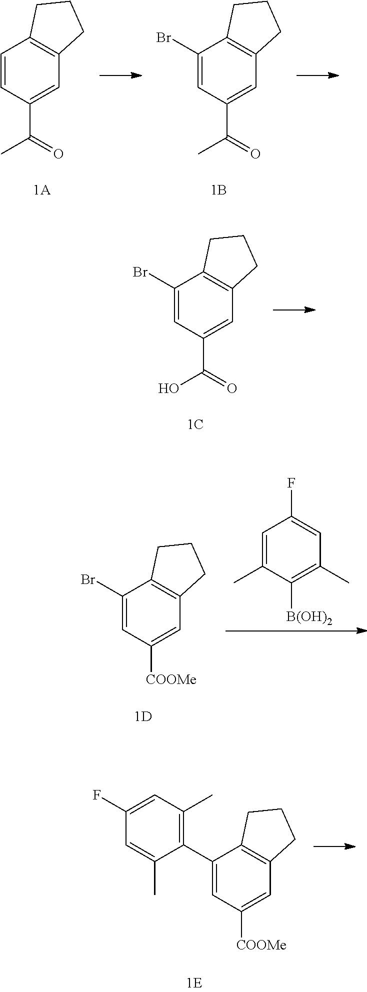

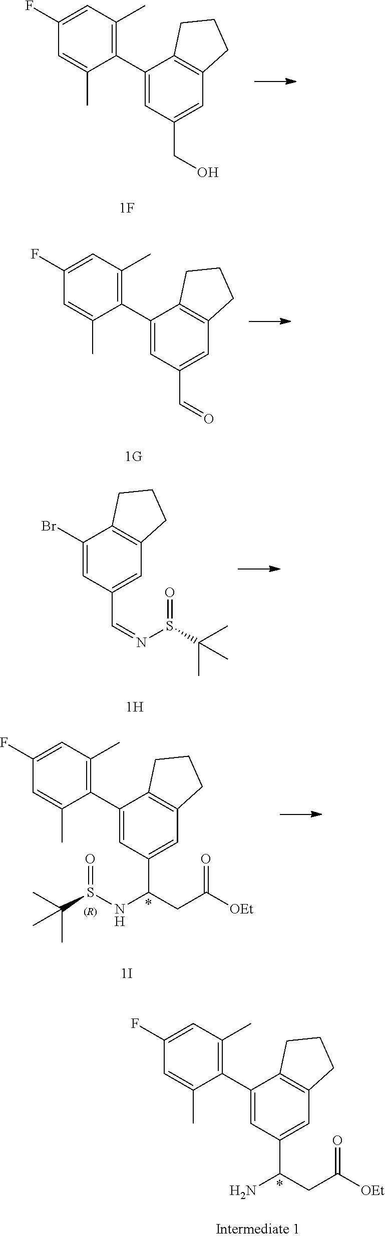

### Step 1: Synthesis of 1B

Under nitrogen protection, intermediate 1a (6.3 g, 39.34 mmol) was dissolved in dichloromethane (150 mL), and anhydrous aluminum chloride (13.11 g, 98.35 mmol) was added. The reaction mixture was reacted at room temperature for 5 min, a solution of bromine (8.80 g, 55.08 mmol) in dichloromethane (50 mL) was added, and the resulting mixture was reacted at room temperature for 2 h. The reaction liquid was poured into ice water (200 mL), dichloromethane (100 mL) was added, and the resulting mixture was washed with saturated sodium chloride aqueous solution (80 mL), dried over anhydrous sodium sulfate, filtered and concentrated. The residue was subjected to silica gel column chromatography to afford 1B (8.0 g, 85.05%).

LC-Ms m/z (ESI): 241.1 [M+H]⁺

### Step 2: Synthesis of 1C

1B (8.1 g, 33.88 mmol) was dissolved in DME (100 mL), 1 M sodium hypochlorite solution (188 mL) and 10 N sodium hydroxide solution (18.5 mL) were added, and the mixture was reacted at 50° C. for 1 h. Water (150 mL) was added, the resulting mixture was extracted with diethyl ether (200 mL), and the aqueous phase was adjusted with hydrochloric acid to pH 1-2 and extracted with diethyl ether (150 ml×2). The organic phases were combined, dried over anhydrous sodium sulfate and concentrated to afford 1C (7.6 g, 93.05%).

LC-Ms m/z (ESI): 240.9 [M+H]⁺

### Step 3: Synthesis of 1D

1C (7.6 g, 31.52 mmol) was dissolved in methanol (50 mL), thionyl chloride (11.25 g, 94.56 mmol) was added, and the mixture was reacted at 70° C. for 2 h. The reaction mixture was concentrated under reduced pressure and subjected to silica gel column chromatography to afford 1D (8.0 g, 99.49%).

LC-Ms m/z (ESI): 255.0 [M+H]⁺

### Step 4: Synthesis of 1E

Under nitrogen protection, 1D (2 g, 7.84 mmol), 4-fluoro2,6-dimethylphenylboronic acid (2.63 g, 15.68 mmol) and cesium carbonate (7.66 g, 23.52 mmol) were added to 1,4-dioxane (30.0 mL) and water (3.0 mL), followed by Pd(PPh₃)₄ (1.81 g, 1.57 mmol), and the mixture was reacted at 100° C. in a sealed tube for 5 h. The reaction system was cooled to room temperature and filtered through celite. Ethyl acetate (50 mL) was added and the mixture was successively washed with water (30 mL) and saturated sodium chloride aqueous solution (30 mL), dried over anhydrous sodium sulfate, filtered and concentrated under reduced pressure. The residue was subjected to flash chromatography on a silica gel column to afford 1E (2.2 g, yield: 94.05%).

### Step 5: Synthesis of 1F

Under nitrogen protection, 1E (2.13 g, 7.14 mmol) was dissolved in dry THF (30 mL) and at 0° C., lithium aluminum hydride (406 mg, 10.71 mmol) was slowly added. The resulting mixture was reacted at room temperature for 1 h. 10% sodium sulfate aqueous solution (20 mL) was added and the mixture was filtered. The solvent was removed under reduced pressure to afford crude 1F.

### Step 6: Synthesis of 1g

Under nitrogen protection, crude 1F (1.87 g, 6.92 mmol) was dissolved in dry dichloromethane (35 mL), sodium bicarbonate (1.16 g, 13.84 mmol) and Dess-Martin periodinane (3.81 g, 9.00 mmol) were added at room temperature, and the resulting mixture was reacted at room temperature for 1 h. Saturated sodium thiosulfate solution (10 mL) and saturated sodium bicarbonate solution (10 mL) were added and the mixture was extracted with dichloromethane (50 mL×3), dried over anhydrous sodium sulfate and filtered. The solvent was removed under reduced pressure and the residue was subjected to silica gel column chromatography to afford 1G (1.63 g, 87.68%).

### Step 7: Synthesis of 1H

Under nitrogen protection, 1G (1.61 g, 6.00 mmol) and R-tert-butylsulfinamide (1.01 g, 9.00 mmol) were dissolved in THF (35 mL), tetraethyl titanate (2.05 g, 9.00 mmol) was slowly added, and the resulting mixture was reacted at 45° C. for 15 h and concentrated under reduced pressure to afford a crude, which was subjected to flash column chromatography on a silica gel column to afford 1H (1.73 g, yield: 77.61%).

LC-Ms m/z (ESI): 329.3 [M+H]⁺

### Step 8: Synthesis of 1I

Zinc powder (915 mg, 14 mmol) was added to dry THF (5 mL), the mixture was subjected to nitrogen replacement three times, CuCl (297 mg, 3 mmol) was added, and the resulting mixture was reacted at 60° C. for 2 h. The reaction mixture was cooled to room temperature, ethyl bromoacetate (835 mg, 5 mmol) was slowly added, and the resulting mixture was reacted at 60° C. for 1 h and cooled to 0° C. A solution of 1H (371 mg, 1 mmol) in THF (1 mL) was added and the mixture was stirred at 0° C. for 3 h. The reaction mixture was filtered through celite and saturated ammonium chloride solution (20 mL) was added. The resulting mixture was extracted with ethyl acetate (30 ml×3), washed with saturated sodium chloride aqueous solution (20 mL), dried over anhydrous sodium sulfate, filtered and concentrated under reduced pressure. The residue was subjected to flash column chromatography to afford 1I (386 mg, yield: 83.98%).

LC-Ms m/z (ESI): 460.2 [M+H]⁺

### Step 9: Synthesis of Intermediate 1

1I (272 mg, 0.27 mmol) was dissolved in THF (2 mL), 4 N hydrogen chloride dioxane solution (2 mL) was added, and the resulting mixture was stirred at room temperature for 3 h. The reaction mixture was concentrated under reduced pressure to afford crude intermediate 1.

LC-Ms m/z (ESI): 356.3 [M+H]⁺

### Intermediate 2: Preparation of Intermediate 2

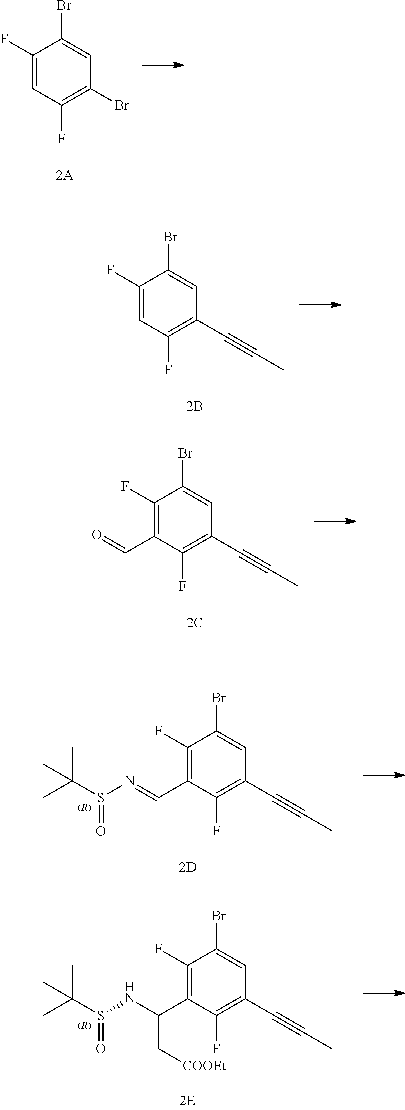

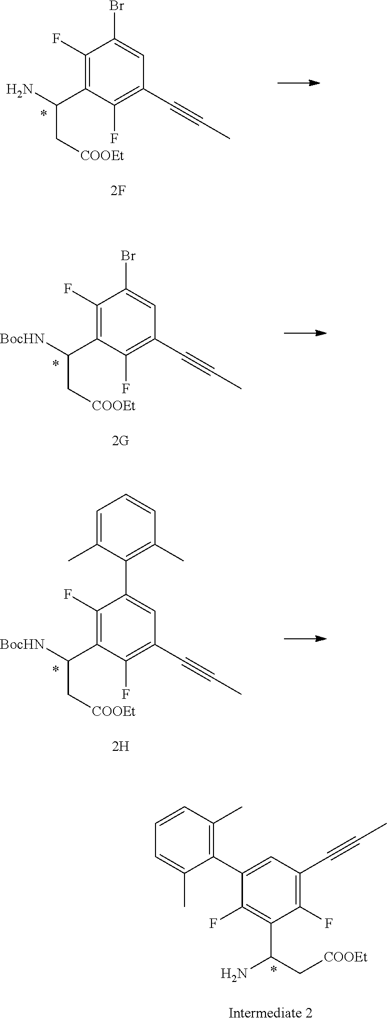 [SMILES: CC#Cc1cc(Br)c(F)c(C(N)CC(=O)OCC)c1F ; CC#Cc1cc(Br)c(F)c(C(CC(=O)OCC)NC(=O)OC(C)(C)C)c1F ; CC#Cc1cc(-c2c(C)cccc2C)c(F)c(C(CC(=O)OCC)NC(=O)OC(C)(C)C)c1F ; CC#Cc1cc(-c2c(C)cccc2C)c(F)c(C(N)CC(=O)OCC)c1F]

### Step 1: Synthesis of 2B

Under nitrogen protection, 2A (25 g, 91.94 mmol), CuI (3.5 g, 18.39 mmol), PdCl₂(PPh₃)₂ (6.45 g, 9.19 mmol), 1-(trimethylsilyl)propyne (9.29 g, 82.75 mmol) and cesium fluoride (41.90 g, 275.82 mmol) were dissolved in THF (250 mL) and the resulting mixture was reacted at 60° C. for 72 h, concentrated under reduced pressure and then subjected to silica gel column chromatography to afford 2B (8.5 g, 40.02%).

### Step 2: Synthesis of 2C

Under nitrogen protection, 2B (8.5 g, 36.79 mmol) was dissolved in THF (75 mL). At −78° C., lithium diisopropylamide (5.91 g, 55.19 mmol) was added and the resulting mixture was stirred for 1 h. DMF (5.38 g, 73.58 mmol) was added dropwise and the mixture was stirred for 30 min. Saturated aqueous ammonium chloride solution (150 mL) was added and the resulting mixture was extracted with ethyl acetate (200 mL×3). The organic phases were combined, dried over anhydrous sodium sulfate and concentrated to afford 2C (7.2 g, 75.55%).

### Step 3: Synthesis of 2D

Under nitrogen protection, 2C (7.2 g, 27.79 mmol) and R-tert-butylsulfinamide (4.04 g, 33.35 mmol) were dissolved in THF (75 mL), tetraethyl titanate (9.51 g, 41.69 mmol) was slowly added, and the resulting mixture was reacted at 45° C. for 3 h and concentrated under reduced pressure to afford a crude, which was subjected to flash column chromatography on a silica gel column to afford 2D (6.80 g, yield: 67.55%).

### Step 4: Synthesis of 2E

Zinc powder (17.19 g, 262.78 mmol) was added to dry THF (45 mL), the mixture was subjected to nitrogen replacement three times, CuCl (5.57 g, 56.31 mmol) was added, and the resulting mixture was reacted at 60° C. for 2 h. The reaction mixture was cooled to room temperature, ethyl bromoacetate (15.67 g, 93.85 mmol) was slowly added, and the resulting mixture was reacted at 60° C. for 1 h and cooled to 0° C. A solution of 2D (6.8 g, 18.77 mmol) in THF (10 mL) was added and the mixture was stirred at 0° C. for 3 h. The reaction mixture was filtered through celite and saturated ammonium chloride solution (200 mL) was added. The resulting mixture was extracted with ethyl acetate (300 ml×3), washed with saturated sodium chloride aqueous solution (200 mL), dried over anhydrous sodium sulfate, filtered and concentrated under reduced pressure. The residue was subjected to flash column chromatography to afford 2E (7.0 g, yield: 82.81%).

### Step 5: Synthesis of 2F

2E (3.0 g, 6.66 mmol) was dissolved in dichloromethane (10 mL), 4 N hydrogen chloride dioxane solution (5 mL) was added, and the resulting mixture was stirred at room temperature for 3 h. The mixture was concentrated under reduced pressure to afford crude 2F.

### Step 6: Synthesis of 2G

Under nitrogen protection, crude 2F (2.0 g, 5.78 mmol) was dissolved in dry dichloromethane (35 mL), triethylamine (1.75 g, 17.34 mmol) and di-tert-butyl dicarbonate (1.51 g, 6.94 mmol) were added at room temperature, and the resulting mixture was reacted at room temperature for 3 h. The solvent was removed under reduced pressure and the residue was subjected to column chromatography on a silica gel column to afford 2G (1.3 g, 50.40%).

### Step 7: Synthesis of 2H

Under nitrogen protection, 2G (0.45 g, 1.11 mmol), 2,6-dimethylphenylboronic acid (0.50 g, 3.33 mmol), potassium phosphate (0.47 g, 2.21 mmol), and RuPhos Pd G3 (0.19 g, 0.22 mmol) were dissolved in DMF (8 mL) and water (0.8 mL) and the mixture was stirred at 75° C. for 3 h. Water (20 mL) was added and the resulting mixture was extracted with ethyl acetate (30 ml×3), washed with saturated sodium chloride aqueous solution (20 mL×2), dried over anhydrous sodium sulfate, filtered and concentrated under reduced pressure. The residue was subjected to flash column chromatography to afford 2H (0.3 g, yield: 57.38%).

Step 8: Synthesis of intermediate 2

2H (300 mg, 0.64 mmol) was dissolved in dichloromethane (2 mL), 4 N hydrogen chloride dioxane solution (2 mL) was added, and the resulting mixture was stirred at room temperature for 3 h. The reaction mixture was concentrated under reduced pressure to afford crude intermediate 2.

LC-Ms m/z (ESI): 372.2 [M+H]⁺

### Intermediate 4

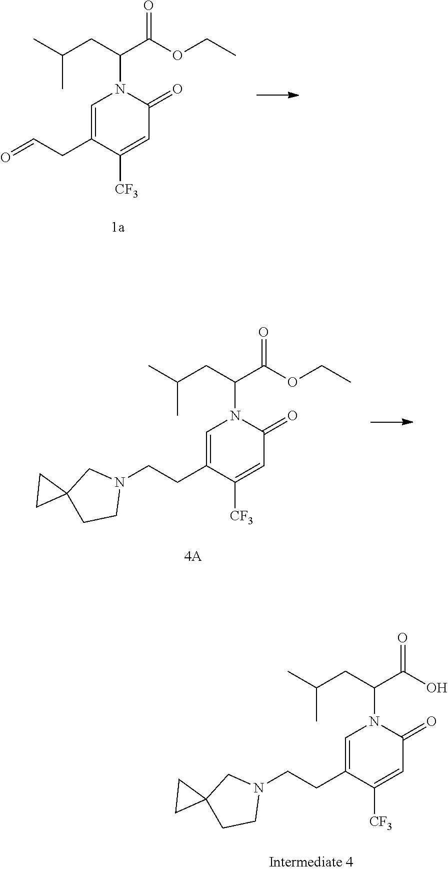 [SMILES: CCOC(=O)C(CC(C)C)n1cc(CC=O)c(C(F)(F)F)cc1=O ; CCOC(=O)C(CC(C)C)n1cc(CCN2CCC3(CC3)C2)c(C(F)(F)F)cc1=O ; CC(C)CC(C(=O)O)n1cc(CCN2CCC3(CC3)C2)c(C(F)(F)F)cc1=O]

### Step 1: Synthesis of 4A

Under nitrogen protection, 1a (1.5 g, 4.32 mmol, referring to WO 2021076890 A1 for synthetic step) and 5-azaspiro[2.4]heptane (0.42 g, 4.32 mmol) were dissolved in 1,2-dichloroethane (10 mL), acetic acid (0.5 mL) was added dropwise, and the mixture was stirred at room temperature for 2 h. Sodium triacetoxyborohydride (1.82 g, 8.64 mmol) was added and the resulting mixture was stirred for 16 h, concentrated under reduced pressure and then subjected to flash column chromatography on a silica gel column to afford 4A (1.0 g, yield: 54.05%).

LC-Ms m/z (ESI): 429.2 [M+H]⁺

Step 2: Synthesis of intermediate 4

4A (1.0 g, 2.34 mmol) was dissolved in THF (3 mL) and water (1 mL), lithium hydroxide monohydrate (112 mg, 4.68 mmol) was added, and the resulting mixture was reacted at room temperature for 5 h. The reaction mixture was adjusted with 1 N hydrochloric acid to pH 5-6, concentrated under reduced pressure and then subjected to column chromatography to afford intermediate 4 (650 mg, 69.55%).

LC-Ms m/z (ESI): 401.2 [M+H]⁺

### Intermediate 5: Preparation of Intermediate 5

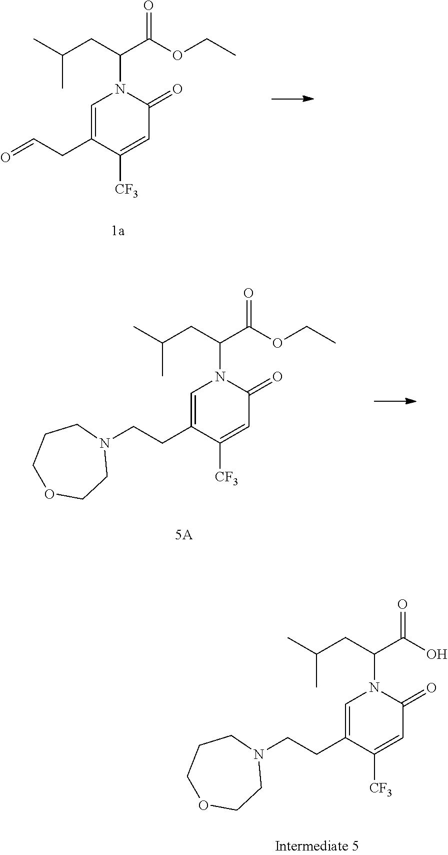 [SMILES: CCOC(=O)C(CC(C)C)n1cc(CC=O)c(C(F)(F)F)cc1=O ; CCOC(=O)C(CC(C)C)n1cc(CCN2CCCOCC2)c(C(F)(F)F)cc1=O ; CC(C)CC(C(=O)O)n1cc(CCN2CCCOCC2)c(C(F)(F)F)cc1=O]

### Step 1: Synthesis of 5A

Under nitrogen protection, 1,4-oxazepane hydrochloride salt (360 mg, 2.59 mmol) was dissolved in 1,2-dichloroethane (8 mL), DIPEA was added, and the resulting mixture was reacted at room temperature for 15 min. 1a (0.9 g, 2.59 mmol, referring to WO 2021076890 A1 for synthetic step) and acetic acid (0.15 mL) were added and the mixture was stirred at room temperature for 1 h. Sodium triacetoxyborohydride (820 mg, 3.86 mmol) was added and the mixture was stirred for 16 h and concentrated under reduced pressure to afford a crude, which was subjected to flash column chromatography on a silica gel column to afford 5A (0.49 g, yield: 43.75%).

LC-Ms m/z (ESI): 433.7 [M+H]⁺

### Step 2: Synthesis of Intermediate 5

5A (0.72 g, 1.66 mmol) was dissolved in ethanol (6 mL) and water (0.6 mL), lithium hydroxide monohydrate (210 mg, 4.98 mmol) was added, and the resulting mixture was reacted at room temperature for 3 h. The reaction mixture was adjusted with 1 N hydrochloric acid to pH 4, concentrated under reduced pressure and then subjected to column chromatography to afford intermediate 5 (530 mg, yield: 78.95%).

LC-Ms m/z (ESI): 405.6 [M+H]⁺

### Intermediate 6: Preparation of Intermediate 6

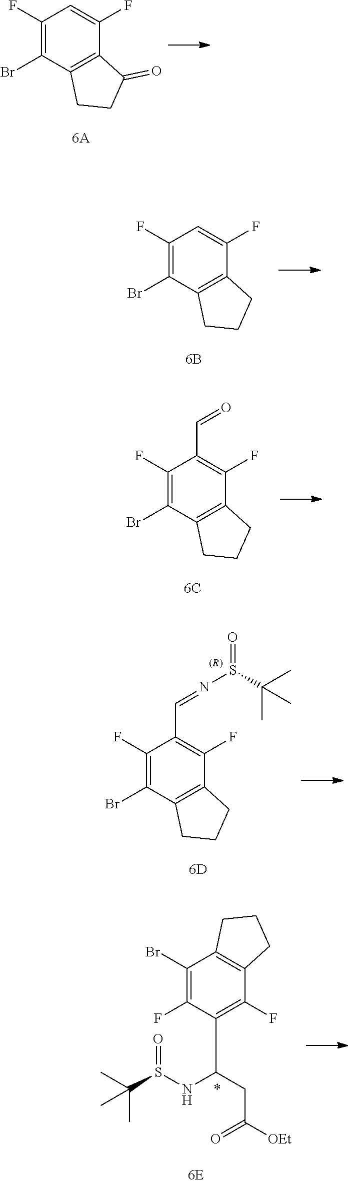

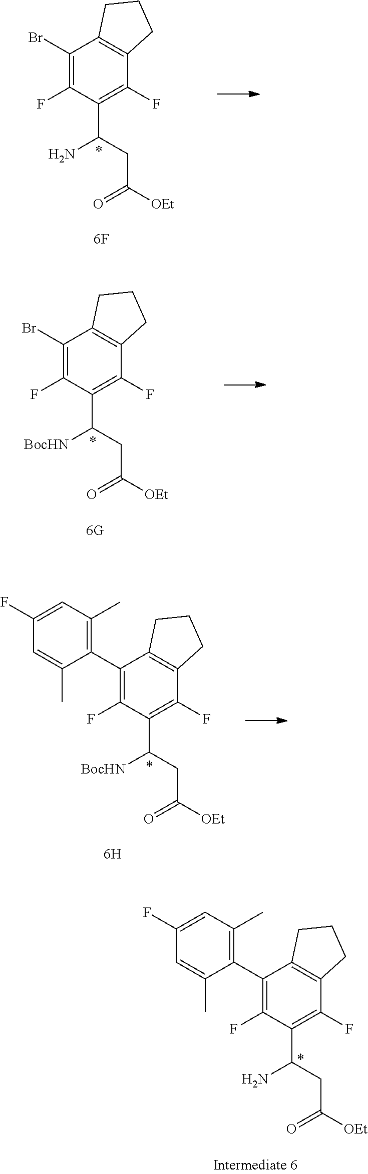 [SMILES: CCOC(=O)CC(N)c1c(F)c(Br)c2c(c1F)CCC2 ; CCOC(=O)CC(NC(=O)OC(C)(C)C)c1c(F)c(Br)c2c(c1F)CCC2 ; CCOC(=O)CC(NC(=O)OC(C)(C)C)c1c(F)c2c(c(-c3c(C)cc(F)cc3C)c1F)CCC2 ; CCOC(=O)CC(N)c1c(F)c2c(c(-c3c(C)cc(F)cc3C)c1F)CCC2]

### Step 1: Synthesis of 6B

6A (1720 mg, 6.96 mmol) was dissolved in trifluoroacetic acid (25 mL), triethylsilane (25 mL) was added, and the mixture was reacted at room temperature for 48 h. Triethylsilane (15 mL) was then added and the resulting mixture was stirred and reacted overnight. The mixture was concentrated under reduced pressure at 30° C., and the residue was separated and purified by column chromatography on a silica gel column to afford crude compound 6B (1700 mg).

### Step 2: Synthesis of 6C

Under nitrogen protection, the crude 6B (1700 mg, 7.29 mmol) was dissolved in dry THF (70 mL), and at −78° C., lithium diisopropylamide (1.56 g, 14.58 mmol) was slowly added. The mixture was stirred at this temperature for 0.5 h and dry DMF (2.6 g, 36.45 mmol) was then added. At −78° C., the mixture was reacted for another 1 h, and the reaction was quenched with ammonium chloride solution (50 mL). The reaction mixture was extracted with ethyl acetate (80 mL×3), dried over anhydrous sodium sulfate and filtered. The solvent was removed under reduced pressure and the residue was separated and purified by column chromatography on a silica gel column to afford 6C (1.58 g, two-step yield: 83.02%).

### Step 3: Synthesis of 6D

Under nitrogen protection, 6C (1580 mg, 6.05 mmol) and R-tert-butylsulfinamide (1100 mg, 9.7 mmol) were dissolved in THF (35 mL), tetraethyl titanate (2070 mg, 9.07 mmol) was slowly added, and the resulting mixture was reacted at 45° C. for 15 h and concentrated under reduced pressure to afford a crude, which was subjected to flash column chromatography on a silica gel column to afford 6D (2100 mg, yield: 95.29%).

LC-Ms m/z (ESI): 364.2[M+H]⁺

### Step 4: Synthesis of 6E

Zinc powder (1.2 g, 18.34 mmol) was added to dry THF (8 mL), the mixture was subjected to nitrogen replacement three times, CuCl (389 mg, 3.93 mmol) was added, and the resulting mixture was reacted at 60° C. for 2 h. The reaction mixture was cooled to room temperature, ethyl bromoacetate (1.09 g, 6.55 mmol) was slowly added, and the resulting mixture was reacted at 60° C. for 1 h and cooled to 0° C. A solution of 6D (478 mg, 1.31 mmol) in THF (3 mL) was added and the mixture was stirred at 0° C. for 3 h. The reaction mixture was filtered through celite and saturated ammonium chloride solution (30 mL) was added. The resulting mixture was extracted with ethyl acetate (40 mL×3), washed with saturated sodium chloride aqueous solution (20 mL), dried over anhydrous sodium sulfate, filtered and concentrated under reduced pressure. The residue was subjected to flash column chromatography to afford 6E (498 mg, yield: 84.04%).

LC-Ms m/z (ESI): 452.4[M+H]⁺

### Step 5: Synthesis of 6F

6E (448 mg, 0.99 mmol) was dissolved in dichloromethane (3 mL), 4 N hydrogen chloride dioxane solution (2.5 mL) was added, and the resulting mixture was stirred at room temperature for 3 h. The reaction mixture was concentrated under reduced pressure to afford the hydrochloride salt of crude 6F.

LC-Ms m/z (ESI): 348.3 [M+H]⁺

### Step 6: Synthesis of 6G

6F was dissolved in THF (4 mL) and water (4 mL), and sodium carbonate (210 mg, 1.98 mmol) and Boc₂O (240 mg, 1.09 mmol) were added and stirred at room temperature for 3 h. Water (10 mL) was added and the resulting mixture was extracted with ethyl acetate (20 mL×3), dried over anhydrous sodium sulfate, filtered and concentrated under reduced pressure. The residue was subjected to flash column chromatography to afford 6G (420 mg, two-step yield: 86.31%).

LC-Ms m/z (ESI): 350.0 [M+H-Boc]⁺

### Step 7: Synthesis of 6H

Under nitrogen protection, 6G (230 mg, 0.51 mmol), 2,6-dimethyl-4-fluorophenylboronic acid (128 mg, 0.77 mmol) and potassium phosphate (325 mg, 1.53 mmol) were added to 1,4-dioxane (2.5 mL) and water (0.25 mL), followed by Xphos-G2-Pd (40 mg, 0.051 mmol), and the resulting mixture was reacted at 100° C. in a sealed tube for 24 h. The reaction system was cooled to room temperature. Ethyl acetate (40 mL) was added and the mixture was successively washed with water (20 mL) and saturated sodium chloride aqueous solution (20 mL), dried over anhydrous sodium sulfate, filtered and concentrated under reduced pressure. The residue was subjected to flash chromatography on a silica gel column to afford 6H (210 mg, yield: 83.77%).

LC-Ms m/z (ESI): 392.6 [M+H-Boc]⁺

Step 8: Synthesis of intermediate 6

6H (210 mg, 0.43 mmol) was dissolved in THF (3 mL), 4 N hydrogen chloride dioxane solution (2.5 mL) was added, and the resulting mixture was stirred at room temperature for 3 h. The reaction mixture was concentrated under reduced pressure to afford the hydrochloride salt of crude intermediate 6.

LC-Ms m/z (ESI): 392.6[M+H]⁺

### Intermediate 7: Preparation of Intermediate 7

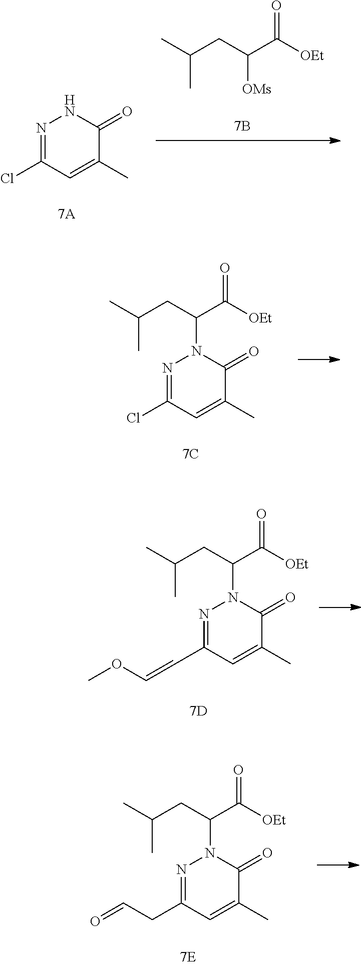

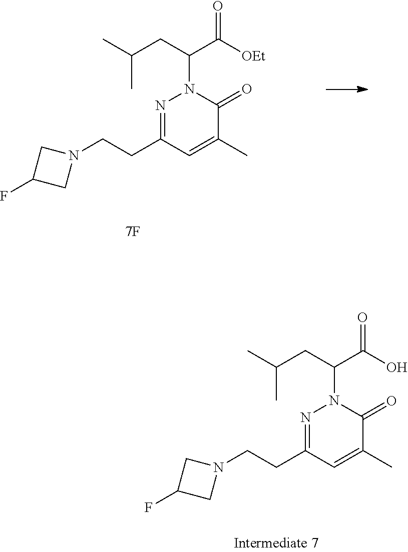 [SMILES: CCOC(=O)C(CC(C)C)n1nc(CCN2CC(F)C2)cc(C)c1=O ; Cc1cc(CCN2CC(F)C2)nn(C(CC(C)C)C(=O)O)c1=O]

### Step 1: Synthesis of 7C

Under nitrogen protection, 7A (1.0 g, 6.92 mmol) and 7B (2.14 g, 9.0 mmol) were dissolved in ultra-dry DMF (42 mL) and potassium carbonate (1.91 g, 13.84 mmol) was added. The mixture was reacted under a nitrogen atmosphere at 80° C. for 4 h. 100 mL of ethyl acetate was added to dilute the system, and the organic phase was washed with water (50 mL×3), followed by saturated sodium chloride aqueous solution (50 mL), and dried over anhydrous sodium sulfate, filtered and concentrated. The residue was subjected to silica gel column chromatography to afford 7C (2.18 g, 99%).

LC-Ms m/z (ESI): 287.1 [M+H]⁺

### Step 2: Synthesis of 7D

7C (2 g, 6.92 mmol) and (E)-2-(2-ethoxyvinyl)-4,4,5,5-tetramethyl-1,3,2-dioxaborolane (2.06 g, 10.38 mmol) were dissolved in a mixed solution of toluene (30 mL) and water (6 mL), and sodium carbonate (2.2 g, 20.76 mmol) and PdCl2(dppf) (506 mg, 0.692 mmol) were added. Reaction was carried out under nitrogen atmosphere at 100° C. overnight. 50 mL of water was added to dilute the system, and extraction was performed by addition of ethyl acetate (80 mL×3). The organic phase was washed with saturated sodium chloride aqueous solution (50 mL), dried over anhydrous sodium sulfate, filtrated, concentrated and subjected to silica gel column chromatography to afford 7D (2.27 g, 99%).

LC-Ms m/z (ESI): 309.1 [M+H]⁺

### Step 3: Synthesis of 7E

7D (2.27 g, 6.92 mmol) was dissolved in dichloromethane (50 mL), trifluoroacetic acid (15 mL) was added, and the resulting mixture was reacted at room temperature for 2 h. The reaction mixture was concentrated under reduced pressure to afford crude intermediate 7E.

LC-Ms m/z (ESI): 295.1 [M+H]⁺

### Step 4: Synthesis of 7F

Under nitrogen protection, the crude 7E was dissolved in 1,2-dichloroethane (20 mL) and methanol (20 mL). 3-Fluoroazetidine hydrochloride salt (780 mg, 10.38 mmol) and acetic acid (0.5 mL) were added, and the mixture was stirred at room temperature for 1 h. Sodium triacetoxyborohydride (4.38 g, 20.76 mmol) was added and the reaction was carried out overnight. The system was diluted with 100 mL of water, and saturated sodium bicarbonate solution was added to adjusted pH=9-10. The mixture was extracted with dichloromethane (100 mL×3), and the organic phase was washed with saturated sodium chloride aqueous solution (50 mL). The mixture was dried over anhydrous sodium sulfate, filtrated, concentrated, and subjected to silica gel column chromatography to afford 7F (1.25 g, 48% over two steps).

LC-Ms m/z (ESI): 354.2 [M+H]⁺

Step 5: Synthesis of intermediate 7

7F (1.25 g, 3.54 mmol) was dissolved in ethanol (15 mL) and water (1.5 mL), lithium hydroxide monohydrate (446 mg, 10.62 mmol) was added, and the resulting mixture was reacted at room temperature for 3 h. The reaction mixture was adjusted with 1 N hydrochloric acid to pH 4, concentrated under reduced pressure and then subjected to column chromatography to afford intermediate 7 (700 mg, 61%).

LC-Ms m/z (ESI): 362.2 [M+H]⁺

### Example 1 Preparation of Compound 1

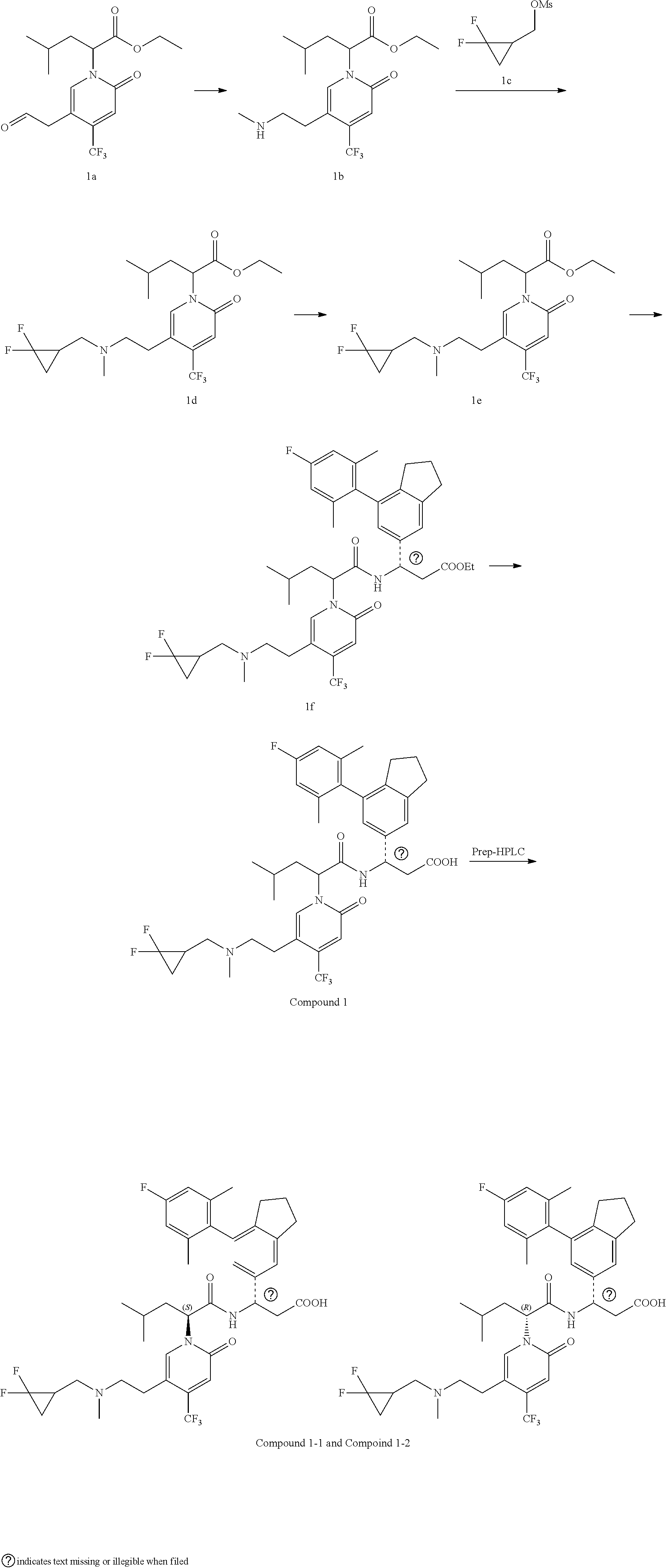

### Step 1: Preparation of 1b

Under nitrogen protection, 1a (3.0 g, 8.64 mmol, referring to WO 2021076890 A1 for synthetic step) and methanamine hydrochloride salt (1.16 g, 17.28 mmol) were dissolved in 1,2-dichloroethane (30 mL). 5 drops of acetic acid were added dropwise and the mixture was stirred at room temperature for 2 h. Sodium triacetoxyborohydride (3.65 g, 17.28 mmol) was added and the resulting mixture was stirred for another 16 h, and concentrated under reduced pressure to afford a crude, which was then subjected to flash column chromatography on a silica gel column to afford 1b (1.0 g, yield: 31.95%).

LC-Ms m/z (ESI): 363.2 [M+H]⁺

### Step 2: Preparation of 1d

Under nitrogen protection, 1b (1.0 g, 2.76 mmol) and 1c (1.02 g, 5.52 mmol) were dissolved in DMF (30 mL), potassium carbonate (1.14 g, 8.28 mmol) was added, and the resulting mixture was stirred at 601C for 24 h and concentrated under reduced pressure to afford a crude, which was subjected to flash column chromatography on a silica gel column to afford 1d (1.0 g, yield: 80.08%).

LC-Ms m/z (ESI): 453.2 [M+H]⁺

### Step 3: Preparation of 1e

1d (1.0 g, 2.21 mmol) was dissolved in THF (3 mL) and water (1 mL), lithium hydroxide monohydrate (112 mg, 4.68 mmol) was added, and the resulting mixture was reacted at room temperature for 5 h. The reaction mixture was adjusted with 1 N hydrochloric acid to pH 5-6, concentrated under reduced pressure and then subjected to column chromatography to afford 1e (700 mg, 74.62%).

LC-Ms m/z (ESI): 425.2 [M+H]⁺

### Step 4: Preparation of 1f

Under nitrogen protection, 1e (280 mg, 0.66 mmol) was dissolved in dry DMF (1.5 mL), and HATU (376 mg, 0.99 mmol) and DIPEA (225 mg, 1.74 mmol) were added. At room temperature, the reaction mixture was stirred for 40 min, and then intermediate 1 (234 mg, 0.66 mmol) was added. The resulting mixture was reacted at room temperature overnight. Ethyl acetate (80 mL) was added and the resulting mixture was successively washed with water (20 mL×2) and saturated sodium chloride aqueous solution (20 mL×1), dried over anhydrous sodium sulfate and filtered and the filtrate was concentrated under reduced pressure and subjected to column chromatography on a silica gel column to afford 1f (300 mg).

LC-Ms m/z (ESI): 762.4 [M+H]⁺

### Step 5: Preparation of Compounds 1-1 and 1-2

1f (300 mg) was dissolved in THF (3 mL) and water (1 mL), lithium hydroxide monohydrate (45 mg, 1.08 mmol) was added, and the resulting mixture was reacted at room temperature for 5 h. PH was adjusted to 5-6 with 1 N hydrochloric acid, and the resulting solution was concentrated under reduced pressure to afford crude compound 1, which was separated and purified by prep-HPLC (method 1) to afford compound 1-1 (15 mg, Rt=4.325 min, yield: 5.19%) and compound 1-2 (15 mg, Rt=4.429 min, yield: 5.19%).

### Compound 1-1:

LC-Ms m/z (ESI): 734.3 [M+H]⁺

¹H NMR (400 MHz, Methanol-d₄) δ 7.93 (s, 1H), 7.17 (s, 1H), 6.84-6.72 (m, 4H), 5.75 (t, 1H), 5.34-5.28 (m, 1H), 3.54-3.43 (m, 1H), 3.40-3.33 (m, 2H), 3.09-2.95 (m, 5H), 2.95-2.89 (m, 2H), 2.86-2.80 (m, 2H), 2.44-2.36 (m, 2H), 2.19-2.08 (m, 1H), 2.02-1.93 (m, 4H), 1.88 (s, 3H), 1.80 (s, 4H), 1.55-1.41 (m, 2H), 1.01-0.91 (m, 6H).

### Compound 1-2:

LC-Ms m/z (ESI): 734.3 [M+H]⁺

¹H NMR (400 MHz, Methanol-d₄) δ 7.93 (s, 1H), 7.22 (s, 1H), 6.89 (s, 1H), 6.85 (s, 1H), 6.84-6.79 (m, 2H), 5.73 (t, 1H), 5.31 (t, 1H), 3.53-3.45 (m, 1H), 3.41-3.33 (m, 3H), 3.08-2.95 (m, 7H), 2.89-2.76 (m, 2H), 2.49-2.42 (m, 2H), 2.21-2.09 (m, 1H), 2.06-2.00 (m, 2H), 1.94 (s, 6H), 1.85-1.78 (m, 3H), 1.54-1.45 (m, 1H), 1.38-1.27 (m, 1H), 0.92-0.83 (m, 6H).

### Example 2: Preparation of Compound 2

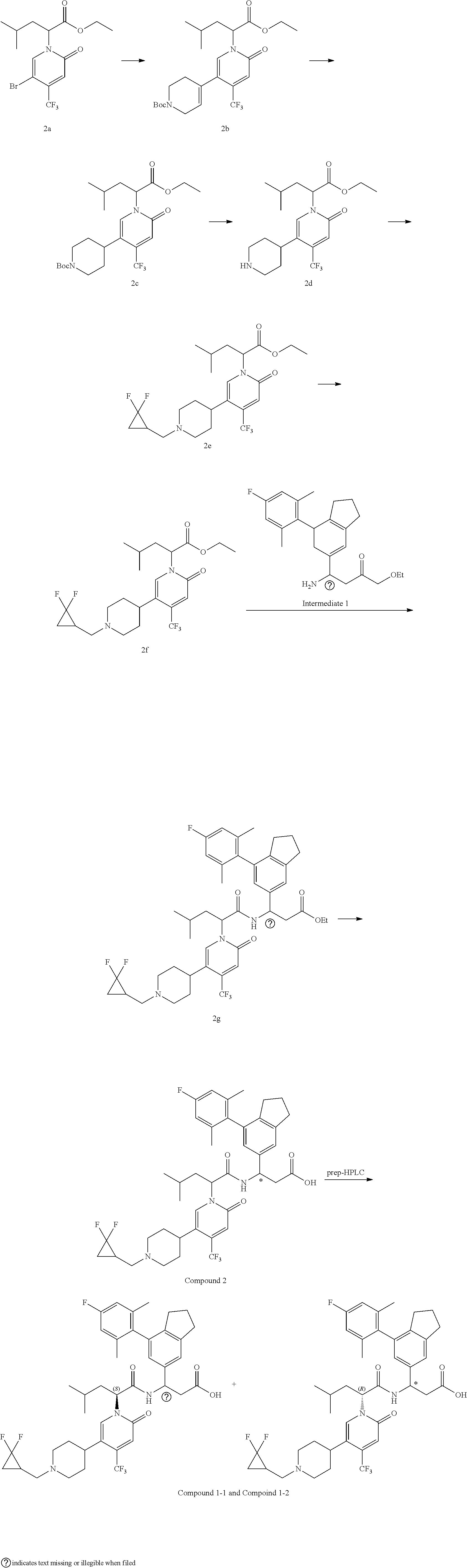

### Step 1: Synthesis of 2b

Under nitrogen protection, 2a (6.0 g, 15.62 mmol, referring to WO 2021076890 A1 for synthetic step) was dissolved in 1,4-dioxane (60 mL) and water (6 mL), N-Boc-1,2,5,6-tetrahydropyridine-4-boronic acid pinacol ester (5.8 g, 18.74 mmol), Pd(PPh₃)₄ (1.8 g, 1.56 mmol) and potassium carbonate (6.48 g, 46.86 mmol) were successively added, and the mixture was reacted at 70° C. for 30 h. After the reaction was complete, extraction was performed by addition of ethyl acetate (180 mL) and the resulting mixture was washed with water (50 mL×2) and saturated sodium chloride aqueous solution (50 mL×1), dried over anhydrous sodium sulfate and filtered and the filtrate was concentrated under reduced pressure and subjected to column chromatography on a silica gel column to afford 2b (5.0 g, yield: 65.79%).

LC-Ms m/z (ESI): 487.3 [M+H]⁺

### Step 2: Synthesis of 2c

Under hydrogen conditions, 2b (5.0 g, 10.28 mmol) was dissolved in methanol (50 mL), an equal mass of Pd/C was added, and the reaction was carried out for 2 h at room temperature. The solid was removed by filtration and the filtrate was concentrated and purified by column chromatography to afford 2c (2.5 g, yield: 49.78%).

LC-Ms m/z (ESI): 489.3 [M+H]⁺

### Step 3: Synthesis of 2d

2c (480.0 mg, 0.98 mmol) was dissolved in dichloromethane (5 mL), hydrogen chloride-1,4-dioxane (3 mL) was added, and the reaction was carried out for 1 h. After the reaction was complete, the solvent was removed under reduced pressure to afford crude compound 2d.

LC-Ms m/z (ESI): 389.2 [M+H]⁺

### Step 4: Synthesis of 2e

2d (350.0 mg, 0.90 mmol) was dissolved in methanol (4 mL), triethylamine (273.21 mg, 2.7 mmol) and methyl (2,2-difluorocyclopropyl)methanesulfonate (201.07 mg, 1.08 mmol) were added sequentially and the reaction was carried out at 60° C. for 16 h. After the reaction was complete, the reaction solution was purified by column chromatography to afford 2e (280.0 mg, yield: 65.02%).

LC-Ms m/z (ESI): 465.90 [M+H]⁺

### Step 5: Synthesis of 2f

2e (280.0 mg, 0.60 mmol) was dissolved in methanol (2 mL) and water (1 mL), lithium hydroxide (28.47 mg, 1.20 mmol) was added, and the reaction was carried out at room temperature for 1-2 h. After the reaction was complete, the reaction solution was purified by reverse phase column chromatography to afford 2f (100.0 mg, yield: 37.0%).

LC-Ms m/z (ESI): 451.70 [M+H]⁺

### Step 6: Synthesis of 2g

Under nitrogen protection, intermediate 1 (80.0 mg, 0.23 mmol) was dissolved in dry DMF (1.5 mL), and HOBt (62.16 mg, 0.46 mmol), EDCI (88.18 mg, 0.46 mmol) and DIPEA (118.90 mg, 0.92 mmol) were successively added. At room temperature, the reaction mixture was stirred for 40 min, and then 2e (100.0 mg, 0.23 mmol) was added. The resulting mixture was reacted at room temperature overnight. Ethyl acetate (80 mL) was added and the resulting mixture was successively washed with water (20 mL×2) and saturated sodium chloride aqueous solution (20 mL×1), dried over anhydrous sodium sulfate and filtered and the filtrate was concentrated under reduced pressure and subjected to column chromatography on a silica gel column to afford 2g (50.0 mg, yield: 28.59%).

LC-Ms m/z (ESI): 788.4 [M+H]⁺

### Step 7: Synthesis of Compound 2-1 and Compound 2-2

Compound 2g (50.0 mg, 0.063 mmol) was dissolved in methanol (1 mL) and water (0.5 mL), lithium hydroxide monohydrate (3.02 mg, 0.13 mmol) was added, and the resulting mixture was reacted at room temperature for 30 min. PH was adjusted to 5-6 with 1 N hydrochloric acid and concentrated under reduced pressure to afford crude compound 2, which was separated and purified by prep-HPLC (Method 2) to afford the trifluoroacetate salt of compound 2-1 (4.0 mg, Rt=5.955 min, yield: 8.36%) and the trifluoroacetate salt of compound 2-2 (3.0 mg, Rt=5.989 min, yield: 6.27%).

### Trifluoroacetate Salt of Compound 2-1:

LC-Ms m/z (ESI): 760.3 [M+H]⁺

¹H NMR (400 MHz, Methanol-d₄) δ 9.16 (d, 1H), 7.81 (s, 1H), 7.16 (d, 1H), 6.83-6.74 (m, 3H), 6.72 (d, 1H), 5.76 (t, 1H), 5.38-5.29 (m, 1H), 4.55-4.45 (m, 1H), 3.77-3.61 (m, 2H), 3.50-3.39 (m, 1H), 3.26-3.10 (m, 3H), 3.04-2.79 (m, 6H), 2.45-2.35 (m, 2H), 2.22-2.07 (m, 2H), 2.04-1.93 (m, 4H), 1.88-1.81 (m, 4H), 1.78 (s, 3H), 1.65 (d, 1H), 1.53-1.41 (m, 2H), 1.04-0.91 (m, 6H).

### Trifluoroacetate Salt of Compound 2-2:

LC-Ms m/z (ESI): 760.3 [M+H]⁺

¹H NMR (400 MHz, Methanol-d₄) δ 7.87 (s, 1H), 7.23 (d, 1H), 6.91-6.74 (m, 4H), 5.77-5.70 (m, 1H), 5.36-5.24 (m 1H), 4.56-4.45 (m, 1H), 3.79-3.64 (m, 2H), 3.48-3.39 (m, 1H), 3.26-3.12 (m, 2H), 3.00-2.93 (m, 2H), 2.90-2.76 (m, 2H), 2.49-2.38 (m, 2H), 2.22-1.99 (m, 5H), 1.94 (s, 6H), 1.88-1.78 (m, 2H), 1.77-1.68 (m, 1H), 1.68-1.61 (m, 2H), 1.55-1.43 (m, 1H), 1.37-1.26 (m, 2H), 0.96-0.78 (m, 6H).

### Example 3: Preparation of Compound 3

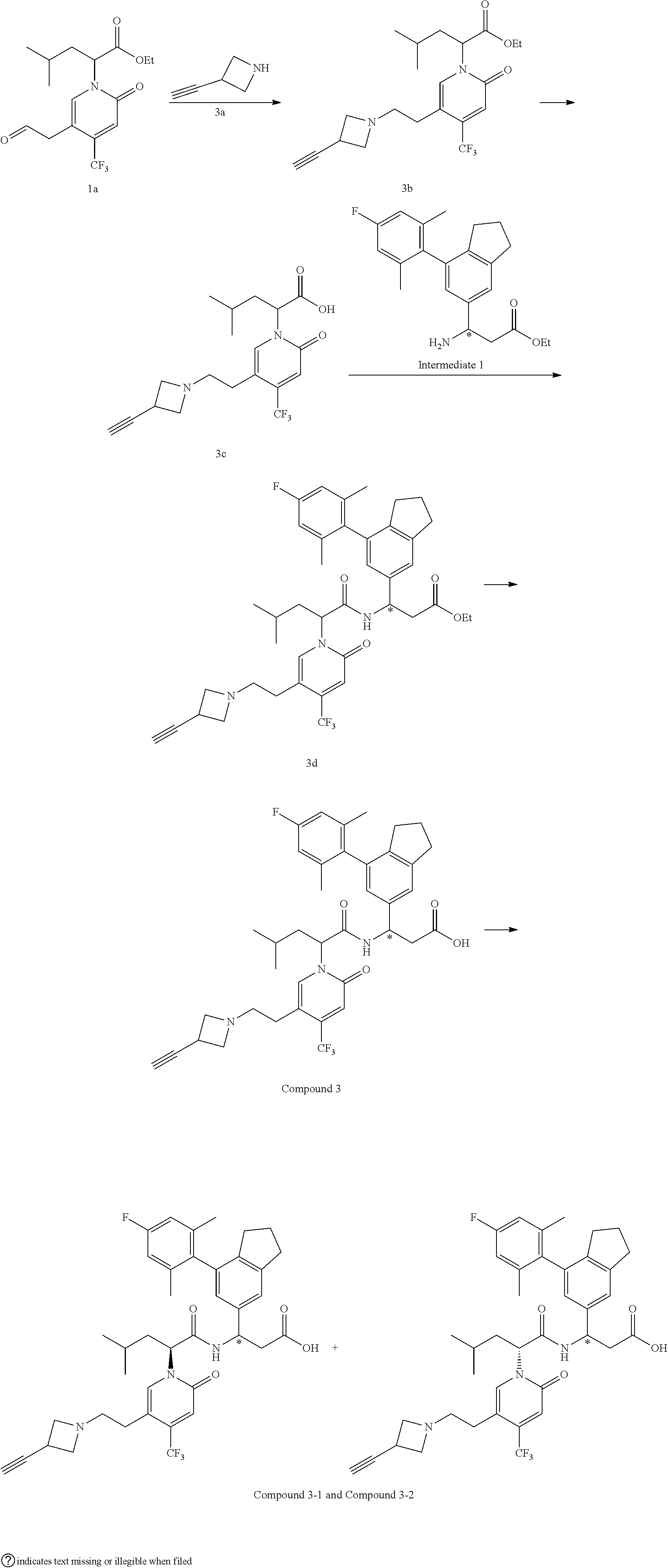

### Step 1: Preparation of 3b

1a (1.4 g crude), 3a (1.2 g, 7.2 mmol) and glacial acetic acid (220 mg, 3.6 mmol) were dissolved in 1,2-dichloroethane (20 mL), stirred at room temperature under nitrogen protection for 30 min, and sodium triacetoxyborohydride (1.5 g, 7.2 mmol) was added. The mixture was stirred at room temperature overnight. 50 mL of ethyl acetate was added, and the mixed solution was successively washed with water (30 mL×1) and saturated sodium chloride aqueous solution (30 mL×1). The organic layer was dried over anhydrous sodium sulfate and filtered, and the filtrate was concentrated under reduced pressure, and the residue was subjected to column chromatography to afford 3b (1.05 g, yield: 71%).

LCMS m/z=413.2 [M+H]⁺

### Step 2: Preparation of 3c

3b (1.05 g, 2.55 mmol) was dissolved in ethanol (15 mL) and water (1.5 mL), lithium hydroxide monohydrate (321 mg, 7.65 mmol) was added, and the reaction was carried out at room temperature for 5 h. The solvent was removed by concentration under reduced pressure, 20 mL of water was added to the residue, and the pH was adjusted to around 2-3 with 1N hydrochloric acid. The aqueous phase was extracted with ethyl acetate (50 mL×2), and the organic phases are combined. The organic phase was washed with saturated sodium chloride aqueous solution (30 mL×1), the organic layer was dried over anhydrous sodium sulfate and filtered, and the filtrate was concentrated under reduced pressure. The residue was subjected to reverse phase (0.1% trifluoroacetic acid) preparative chromatography to afford 3c (410 mg, yield: 42%).

LCMS m/z=385.2 [M+H]⁺;

### Step 3: Preparation of 3d

3c (0.200 g, 0.52 mmol), intermediate 1 (0.18 g, 0.52 mmol) were dissolved in 5 mL DMF, and HOBT (0.14 g, 1.04 mmol), DIPEA (0.27 g, 2.05 mmol) and EDCI (0.20 g, 1.04 mmol) were then added, and the mixed solution was stirred at room temperature for 2 h. 30 ml of ethyl acetate were added, and the resulting solution was washed twice with water (20 ml×2), dried over anhydrous sodium sulfate, and concentrated under reduced pressure, and the residue was subjected to column chromatography to afford the title compound 3d as a yellow oil (0.21 g, yield: 55.95%).

LCMS m/z=722.90 [M+H]⁺;

### Step 4: Preparation of Compounds 3-1 and 3-2

3d (0.18 g, 0.25 mmol) was dissolved in THF (3 mL) and water (1 mL), lithium hydroxide monohydrate (31.5 mg, 0.75 mmol) was added, and the resulting mixture was reacted at room temperature for 5 h. PH was adjusted to 5-6 with 1 N hydrochloric acid, and the resulting solution was concentrated under reduced pressure to afford crude compound 3, which was separated and purified by prep-HPLC (method 1) to afford compound 3-1 (25 mg, Rt=4.300 min, yield: 14%) and compound 3-2 (30 mg, Rt=4.398 min, yield: 17%).

### Compound 3-1:

LC-Ms m/z (ESI): 694.3 [M+H]⁺

¹H NMR (400 MHz, Methanol-d₄) δ 9.16 (d, 1H), 6 7.89 (s, 1H), 7.19 (d, 1H), 6.87-6.73 (m, 4H), 5.80-5.69 (m, 1H), 5.40-5.29 (m, 1H), 4.54-4.33 (m, 2H), 4.29-4.12 (m, 2H), 3.79-3.70 (m, 1H), 3.49-3.35 (m, 2H), 2.98-2.90 (m, 3H), 2.88-2.76 (m, 4H), 2.44-2.37 (m, 2H), 2.04-1.93 (m, 4H), 1.89 (s, 3H), 1.82 (s, 3H), 1.52-1.39 (m, 1H), 1.01-0.92 (m, 6H).

### Compound 3-2:

LC-Ms m/z (ESI): 694.3 [M+H]⁺

¹H NMR (400 MHz, Methanol-d₄) δ 7.89 (s, 1H), 7.24 (d, 1H), 6.93-6.79 (m, 4H), 5.78-5.69 (m, 1H), 5.38-5.29 (m, 1H), 4.57-4.35 (m, 2H), 4.31-4.16 m, 2H), 3.85-3.69 (m, 1H), 3.54-3.35 (m, 2H), 3.07-2.94 (m, 3H), 2.94-2.77 (m, 4H), 2.49-2.41 (m, 2H), 2.08-1.98 (m, 2H), 1.94 (s, 6H), 1.85-1.76 (m, 2H), 1.39-1.27 (m, 1H), 0.93-0.81 (m, 6H).

### Example 4: Preparation of Compound 4

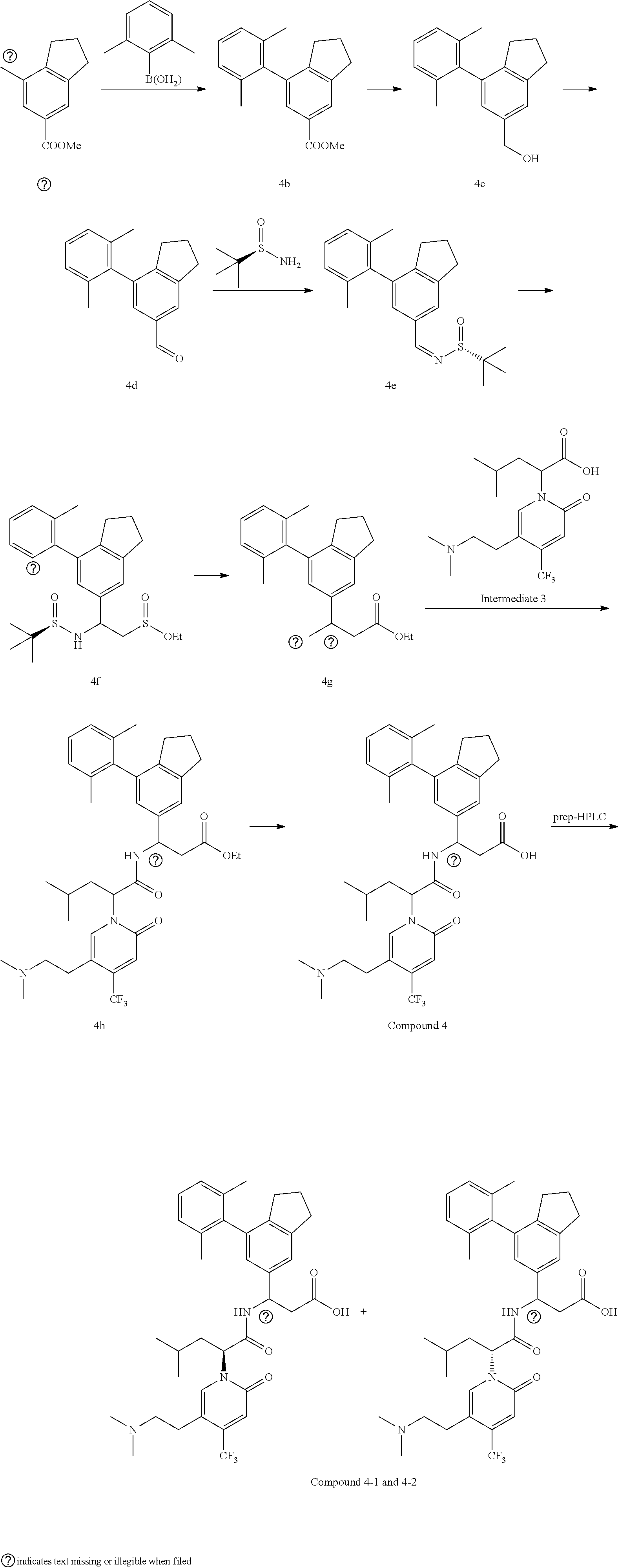

### Step 1: Synthesis of 4b

Under nitrogen protection, 1D (1.8 g, 7.06 mmol), 2,6-dimethylphenylboronic acid (1.27 g, 8.47 mmol) and cesium carbonate (6.90 g, 21.18 mmol) were added to 1,4-dioxane (40.0 mL) and water (4.0 mL), followed by Pd(PPh₃)₄ (1.63 g, 1.41 mmol), and the mixture was reacted at 100° C. in a sealed tube for 5 h. The reaction system was cooled to room temperature and then filtered through celite. Ethyl acetate (50 mL) was added and the mixture was successively washed with water (30 mL) and saturated sodium chloride aqueous solution (30 mL), dried over anhydrous sodium sulfate, filtered and concentrated under reduced pressure. The residue was subjected to flash chromatography on a silica gel column to afford 4b (1.4 g, yield: 70.73%).

### Step 2: Synthesis of 4c

Under nitrogen protection, 4b (1.4 g, 4.99 mmol) was dissolved in dry THF (30 mL) and at 0° C., lithium aluminum hydride (380 mg, 10.03 mmol) was slowly added. The resulting mixture was reacted at room temperature for 1 h. 10% sodium sulfate aqueous solution (20 mL) was added and the mixture was filtered. The solvent was removed under reduced pressure to afford 4c (800 mg, 67.27%).

### Step 3: Synthesis of 4d

Under nitrogen protection, 4c (800 mg, 3.17 mmol) was dissolved in dry dichloromethane (20 mL), sodium bicarbonate (0.53 g, 6.31 mmol) and Dess-Martin periodinane (1.75 g, 4.13 mmol) were added, and the resulting mixture was reacted at room temperature for 1 h. Saturated sodium thiosulfate solution (10 mL) and saturated sodium bicarbonate solution (10 mL) were added and the mixture was extracted with dichloromethane (50 mL×3), dried over anhydrous sodium sulfate and filtered. The solvent was removed under reduced pressure and the residue was subjected to column chromatography on a silica gel column to afford 4d (650 mg, 81.91%).

### Step 4: Synthesis of 4e

Under nitrogen protection, 4d (600 mg, 2.40 mmol) and R-tert-butylsulfinamide (0.35 g, 2.88 mmol) were dissolved in THF (20 mL), tetraethyl titanate (1.64 g, 7.22 mmol) was slowly added, and the resulting mixture was reacted at 45° C. for 15 h and concentrated under reduced pressure to afford a crude, which was subjected to flash column chromatography on a silica gel column to afford 4e (800 mg, yield: 94.29%).

LC-Ms m/z (ESI): 354.5 [M+H]⁺

### Step 5: Synthesis of 4f

Zinc powder (1.81 g, 27.72 mmol) was added to dry THF (5 mL), the mixture was subjected to nitrogen replacement three times, CuCl (590 mg, 5.94 mmol) was added, and the resulting mixture was reacted at 60° C. for 2 h. The reaction mixture was cooled to room temperature, ethyl bromoacetate (1.65 g, 9.9 mmol) was slowly added, and the resulting mixture was reacted at 60° C. for 1 h and cooled to 0° C. A solution of 4e (700 mg, 1.98 mmol) in THF (1 mL) was added and the mixture was stirred at 0° C. for 3 h. The reaction mixture was filtered through celite and saturated ammonium chloride solution (20 mL) was added. The resulting mixture was extracted with ethyl acetate (30 ml×3), washed with saturated sodium chloride aqueous solution (20 mL), dried over anhydrous sodium sulfate, filtered and concentrated under reduced pressure. The residue was subjected to flash column chromatography to afford 4f (750 mg, yield: 85.77%).

LC-Ms m/z (ESI): 442.6 [M+H]⁺

### Step 6: Synthesis of 4g

4f (600 mg, 1.36 mmol) was dissolved in THF (2 mL), 4 N hydrogen chloride dioxane solution (2 mL) was added, and the resulting mixture was stirred at room temperature for 3 h. The reaction mixture was concentrated under reduced pressure to afford crude 4g.

LC-Ms m/z (ESI): 338.4 [M+H]⁺

### Step 7: Synthesis of Compound 4h

Under nitrogen protection, intermediate 3 (260 mg, 0.74 mmol, referring to WO 2021076890 A1 for synthetic step) was dissolved in dry DMF (10 mL), and EDCI (280 mg, 1.46 mmol), HOBT (200 mg, 1.48 mmol) and DIPEA (290 mg, 2.24 mmol) were added. At room temperature, the reaction mixture was stirred for 40 min, and then crude 4g (250 mg, 0.74 mmol) was added. The resulting mixture was reacted at room temperature overnight. Ethyl acetate (80 mL) was added and the resulting mixture was successively washed with water (20 mL×2) and saturated sodium chloride aqueous solution (20 mL×1), dried over anhydrous sodium sulfate and filtered and the filtrate was concentrated under reduced pressure and subjected to column chromatography on a silica gel column to afford 4h (350 mg, yield: 70.83%).

LC-Ms m/z (ESI): 668.8 [M+H]⁺

### Step 8: Synthesis of Compounds 4-1 and 4-2

4h (350 mg, 0.52 mmol) was dissolved in THF (6 mL) and water (2 mL), lithium hydroxide monohydrate (31 mg, 1.29 mmol) was added, and the resulting mixture was reacted at room temperature for 5 h. PH was adjusted to 5-6 with 1 N hydrochloric acid, and the resulting solution was concentrated under reduced pressure to afford crude compound 4, which was separated and purified by prep-HPLC (method 2) to afford the trifluoroacetate salts of compound 4-1 (16 mg, Rt=4.135 min, yield: 4.81%) and compound 4-2 (18 mg, Rt=4.039 min, yield: 5.41%).

### Trifluoroacetate Salt of Compound 4-1:

LC-Ms m/z (ESI): 640.7 [M+H]⁺

¹H NMR (400 MHz, Methanol-d₄) δ 9.15 (d, 1H), 7.92 (s, 1H), 7.22 (s, 1H), 7.15-7.05 (m, 3H), 6.88 (s, 1H), 6.84-6.79 (m, 1H), 5.74 (t, 1H), 5.35-5.28 (m, 1H), 3.66-3.27 (m, 2H), 3.05-2.91 (m, 10H), 2.90-2.77 (m, 2H), 2.49-2.40 (m, 2H), 2.07-1.98 (m, 2H), 1.93 (s, 6H), 1.86-1.78 (m, 2H), 1.39-1.28 (m, 1H), 0.93-0.81 (m, 6H).

### Trifluoroacetate Salt of Compound 4-2:

LC-Ms m/z (ESI): 640.7 [M+H]⁺

¹H NMR (400 MHz, Methanol-d₄) δ 9.17 (d, 1H), 7.92 (s, 1H), 7.16 (d, 1H), 7.13-6.99 (m, 3H), 6.79 (s, 1H), 6.76 (d, 1H), 5.75 (t, 1H), 5.38-5.27 (m, 1H), 3.31-3.19 (m, 2H), 3.06-2.87 (m, 10H), 2.87-2.75 (m, 2H), 2.44-2.34 (m, 2H), 2.04-1.92 (m, 4H), 1.87 (s, 3H), 1.80 (s, 3H), 1.50-1.41 (m, 1H), 1.01-0.90 (m, 6H).

### Example 5: Preparation of Compound 5

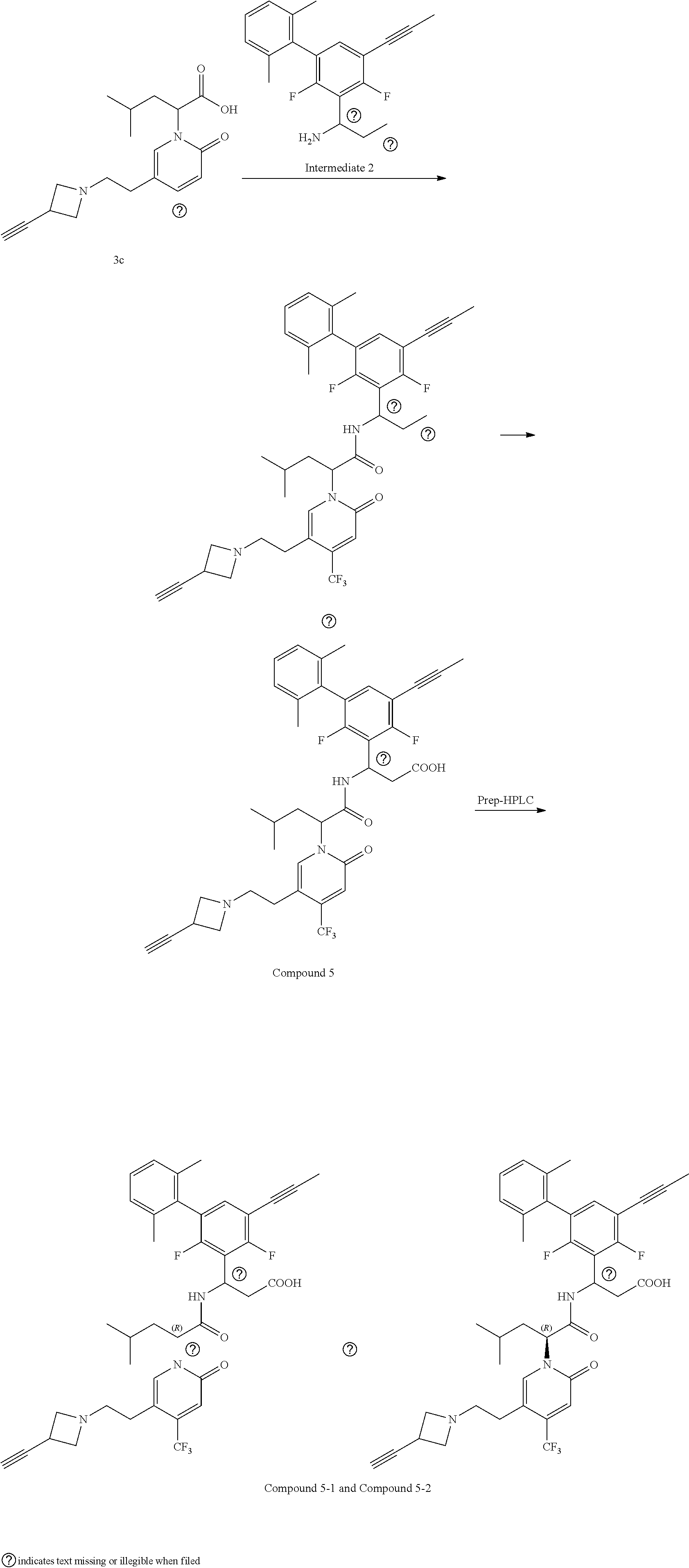

Referring to the preparation method of compound 3, crude compound 5 was obtained, which was separated and purified by prep-HPLC (method 1) to afford compound 5-1 (2 mg, Rt=4.184 min) and compound 5-2 (2 mg, Rt 4.283 min).

### Compound 5-1:

LC-Ms m/z (ESI): 710.3 [M+H]⁺

¹H NMR (400 MHz, Methanol-d₄) δ 7.85 (s, 1H), 7.17 (t, 1H), 7.12-7.02 (m, 3H), 6.83 (s, 1H), 5.83-5.76 (m, 1H), 5.73-5.67 (m, 1H), 5.36-5.32 (m, 1H), 4.46-4.37 (m, 1H), 4.26-4.15 (m, 2H), 3.79-3.72 (m, 1H), 3.49-3.38 (m, 2H), 3.15-3.08 (m, 1H), 2.95-2.93 (m, 1H), 2.89-2.81 (m, 2H), 2.19 (t, 1H), 2.09-2.00 (m, 4H), 1.99-1.91 (m, 4H), 1.89 (s, 3H), 1.65-1.55 (m, 1H), 1.00-0.91 (m, 6H).

### Compound 5-2:

LC-Ms m/z (ESI): 710.3 [M+H]⁺

¹H NMR (400 MHz, Methanol-d₄) δ 7.88 (s, 1H), 7.23-7.03 (m, 4H), 6.89 (s, 1H), 5.84-5.77 (m, 1H), 5.71 (t, 1H), 5.34 (t, 1H), 4.51-4.37 (m, 1H), 4.27-4.16 (m, 1H), 3.82-3.71 (m, 1H), 3.52-3.39 (m, 3H), 3.15-3.03 (m, 2H), 2.97-2.83 (m, 3H), 2.24-2.14 (m, 2H), 2.10-1.95 (m, 7H), 1.92-1.78 (m, 2H), 1.66-1.53 (m, 1H), 0.95-0.79 (m, 6H).

### Example 6: Preparation of Compound 6

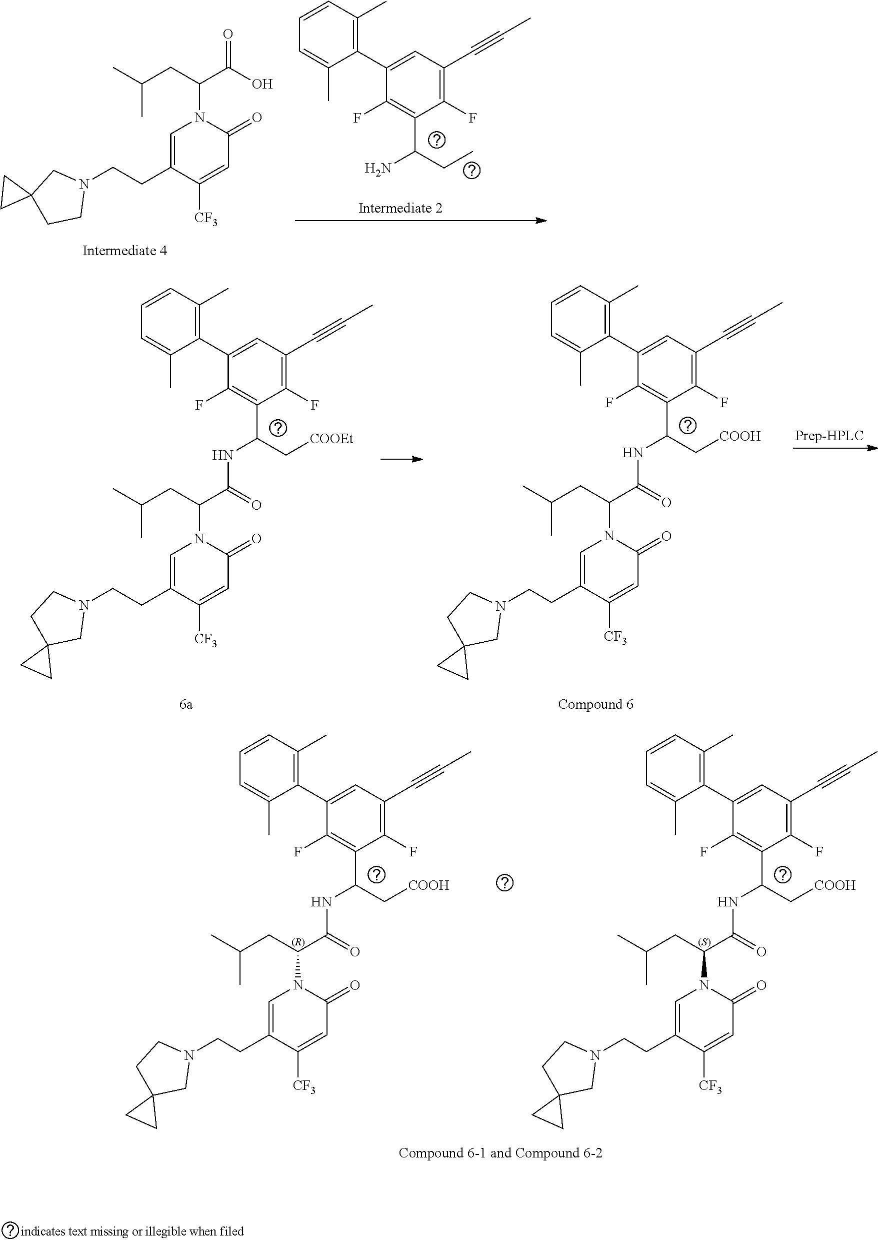

Referring to the preparation method of compound 3, crude compound 6 was obtained, which was separated and purified by prep-HPLC (method 1) to afford compound 6-1 (20 mg, Rt=4.255 min) and compound 6-2 (20 mg, Rt=4.342 min).

### Compound 6-1:

LC-Ms m/z (ESI): 726.3 [M+H]⁺

¹H NMR (400 MHz, Methanol-d₄) δ 7.75 (s, 1H), 7.14 (t, 1H), 7.10-7.01 (m, 2H), 6.96 (t, 1H), 6.78 (s, 1H), 5.77-5.70 (m, 1H), 5.68-5.62 (m, 1H), 2.89-2.80 (m, 1H), 2.81-2.73 (m, 3H), 2.74-2.62 (m, 3H), 2.61-2.51 (m, 4H), 2.02 (s, 3H), 1.99 (s, 3H), 1.97-1.89 (m, 2H), 1.86-1.79 (m, 4H), 1.44-1.36 (m, 1H), 0.98-0.91 (m, 6H), 0.62-0.52 (m, 4H).

### Compound 6-2:

LC-Ms m/z (ESI): 726.3 [M+H]⁺

¹H NMR (400 MHz, Methanol-d₄) δ 8.51 (s, 1H), 7.83 (s, 1H), 7.20-7.15 (m, 1H), 7.13-7.05 (m, 3H), 6.90 (s, 1H), 5.84-5.75 (m, 1H), 5.64 (t, 1H), 3.56-3.47 (m, 2H), 3.44-3.37 (m, 1H), 3.25 (s, 2H), 3.03-2.94 (m, 2H), 2.88-2.78 (m, 1H), 2.60-2.51 (m, 1H), 2.09-1.97 (m, 11H), 1.94-1.84 (m, 1H), 1.77-1.63 (m, 1H), 1.40-1.32 (m, 1H), 0.94-0.85 (m, 6H), 0.85-0.70 (m, 4H).

### Example 7: Preparation of Compound 7

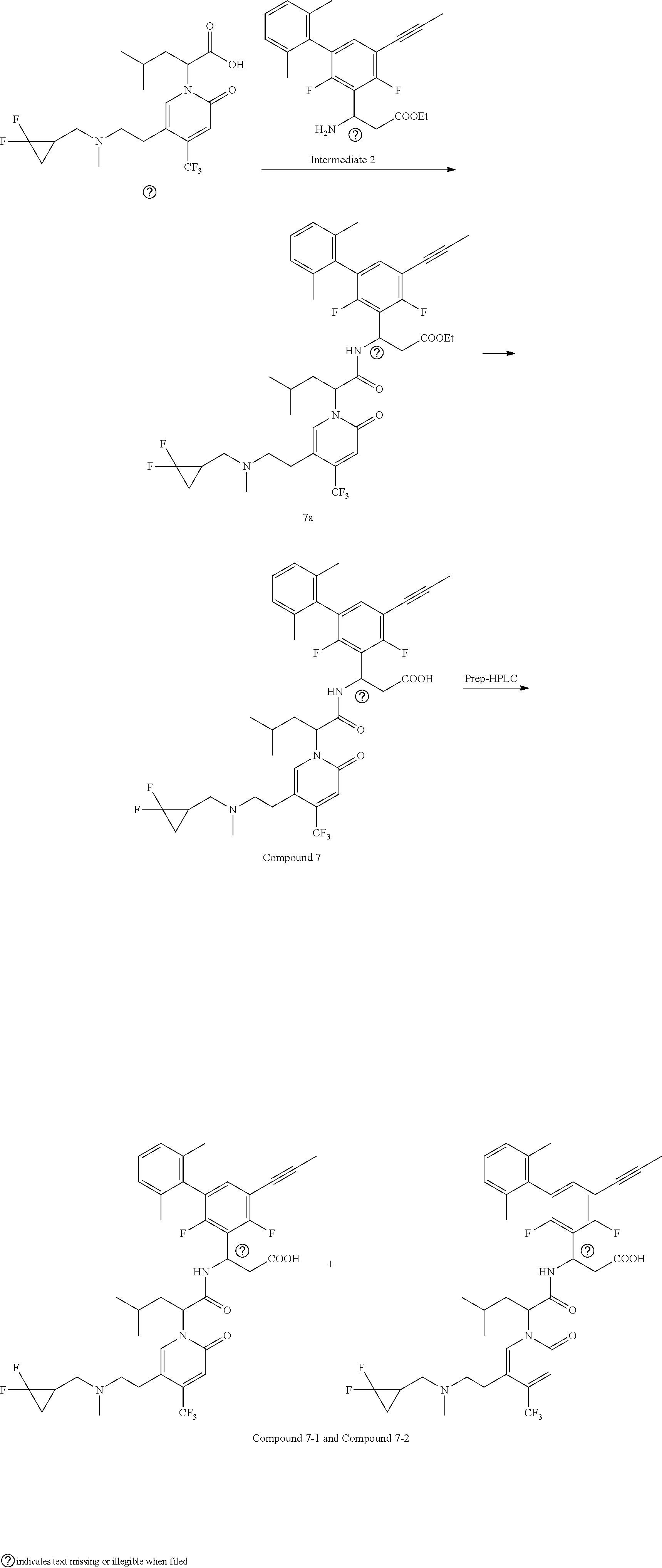

Referring to the preparation method of compound 1, crude compound 7 was obtained, which was separated and purified by prep-HPLC (method 1) to afford compound 7-1 (20 mg, Rt=4.323 min) and compound 7-2 (20 mg, Rt=4.240 min).

### Compound 7-1:

LC-Ms m/z (ESI): 750.3 [M+H]⁺

¹H NMR (400 MHz, Methanol-d₄) δ 7.85 (d, 1H), 7.20-7.13 (m, 1H), 7.12-7.01 (m, 3H), 6.80 (s, 1H), 5.77 (t, 1H), 5.65 (t, 1H), 3.34 (s, 3H), 2.88-2.55 (m, 6H), 2.49-2.38 (m, 1H), 2.36-2.30 (m, 2H), 2.10-1.94 (m, 9H), 1.82-1.75 (m, 2H), 1.55-1.44 (m, 1H), 1.32-1.23 (m, 1H), 1.10-1.01 (m, 1H), 0.93-0.80 (m, 6H).

### Compound 7-2:

LC-Ms m/z (ESI): 750.3 [M+H]⁺

¹H NMR (400 MHz, Methanol-d₄) δ 7.75 (d, 1H), 7.14 (t, 1H), 7.09-7.01 (m, 2H), 6.96 (t, 1H), 6.78 (s, 1H), 5.74 (t, 1H), 5.69-5.60 (m, 1H), 3.34 (s, 3H), 2.91-2.82 (m, 1H), 2.74-2.51 (m, 5H), 2.48-2.34 (m, 1H), 2.35-2.28 (m, 2H), 2.02 (s, 3H), 1.98 (s, 3H), 1.93 (t, 1H), 1.84 (s, 3H), 1.77-1.68 (m, 1H), 1.53-1.35 (m, 2H), 1.09-0.99 (m, 1H), 0.98-0.85 (m, 6H).

### Example 8: Preparation of Compound 8

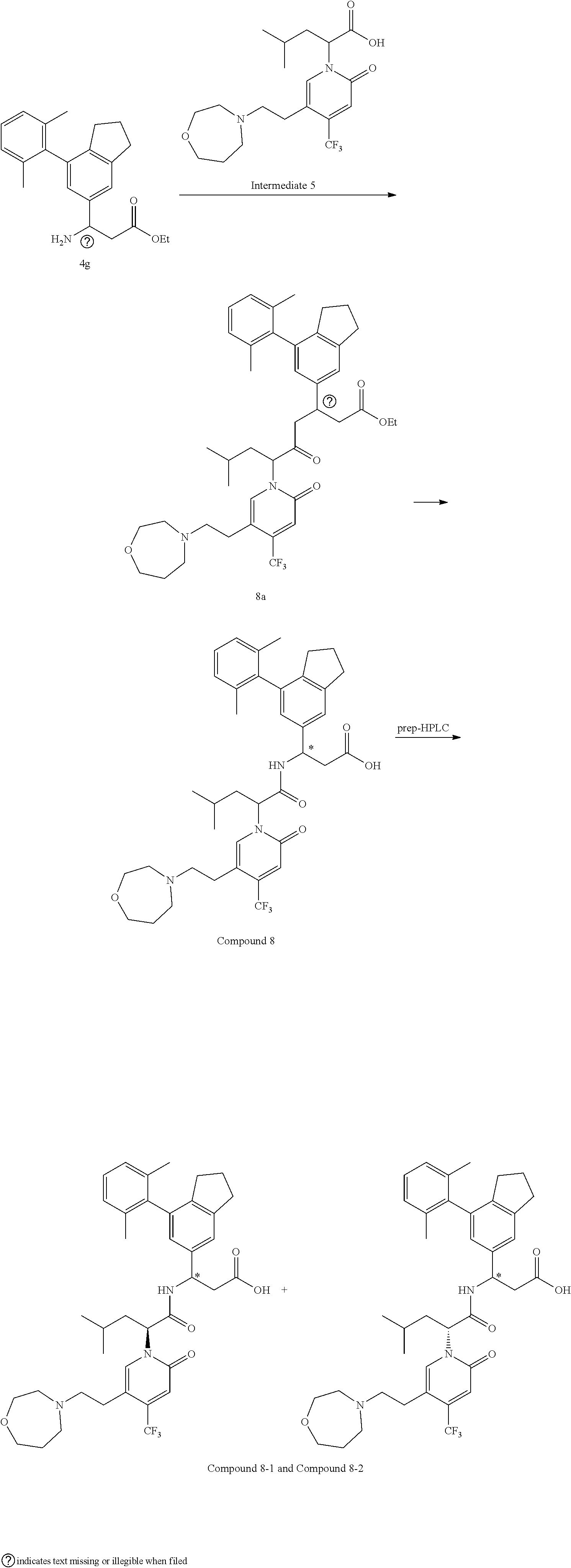 [SMILES: CCOC(=O)CC(N)c1cc2c(c(-c3c(C)cccc3C)c1)CCC2 ; CC(C)CC(C(=O)O)n1cc(CCN2CCCOCC2)c(C(F)(F)F)cc1=O ; CCOC(=O)CC(CC(=O)C(CC(C)C)n1cc(CCN2CCCOCC2)c(C(F)(F)F)cc1=O)c1cc2c(c(-c3c(C)cccc3C)c1)CCC2 ; Cc1cccc(C)c1-c1cc(C(CC(=O)O)NC(=O)C(CC(C)C)n2cc(CCN3CCCOCC3)c(C(F)(F)F)cc2=O)cc2c1CCC2 ; Cc1cccc(C)c1-c1cc(C(CC(=O)O)NC(=O)[C@H](CC(C)C)n2cc(CCN3CCCOCC3)c(C(F)(F)F)cc2=O)cc2c1CCC2 ; Cc1cccc(C)c1-c1cc(C(CC(=O)O)NC(=O)[C@@H](CC(C)C)n2cc(CCN3CCCOCC3)c(C(F)(F)F)cc2=O)cc2c1CCC2]

Referring to the preparation method of compound 4, crude compound 8 was obtained, which was separated and purified by prep-HPLC (method 2) to afford the trifluoroacetate salt of compound 8-1 (25 mg, Rt=4.269 min, yield: 6.53%) and the trifluoroacetate salt of compound 8-2 (28 mg, Rt=4.171 min, yield: 7.32%).

### Trifluoroacetate Salt of Compound 8-1:

LC-Ms m/z (ESI): 696.7 [M+H]⁺

¹H NMR (400 MHz, Methanol-d₄) δ 7.90 (s, 1H), 7.21 (s, 1H), 7.15-7.02 (m, 3H), 6.87 (s, 1H), 6.83 (d, 1H), 5.74-5.66 (m, 1H), 5.34 (t, 1H), 3.93-3.87 (m, 2H), 3.83 (t, 2H), 3.49-3.42 (m, 2H), 3.42-3.37 (m, 2H), 3.35-3.30 (m, 2H), 3.05-2.90 (m, 4H), 2.80-2.74 (m, 2H), 2.45 (t, 2H), 2.19-2.09 (m, 2H), 2.08-1.96 (m, 2H), 1.93 (s, 6H), 1.89-1.74 (m, 2H), 1.39-1.28 (m, 1H), 0.92-0.84 (m, 6H).

### Trifluoroacetate Salt of Compound 8-2:

LC-Ms m/z (ESI): 696.7 [M+H]⁺

¹H NMR (400 MHz, Methanol-d₄) δ 7.92 (s, 1H), 7.16 (s, 1H), 7.13-6.99 (m, 3H), 6.81-6.72 (m, 2H), 5.78-5.70 (m, 1H), 5.37-5.28 (m, 1H), 3.95-3.87 (m, 2H), 3.86-3.77 (m, 2H), 3.48-3.42 (m, 2H), 3.42-3.37 (m, 2H), 3.30-3.20 (m, 2H), 3.06-2.88 (m, 4H), 2.87-2.77 (m, 2H), 2.43-2.36 (m, 2H), 2.17-2.09 (m, 2H), 2.03-1.91 (m, 4H), 1.87 (s, 3H), 1.80 (s, 3H), 1.51-1.39 (m, 1H), 0.99-0.91 (m, 6H).

### Example 9: Preparation of Compound 9

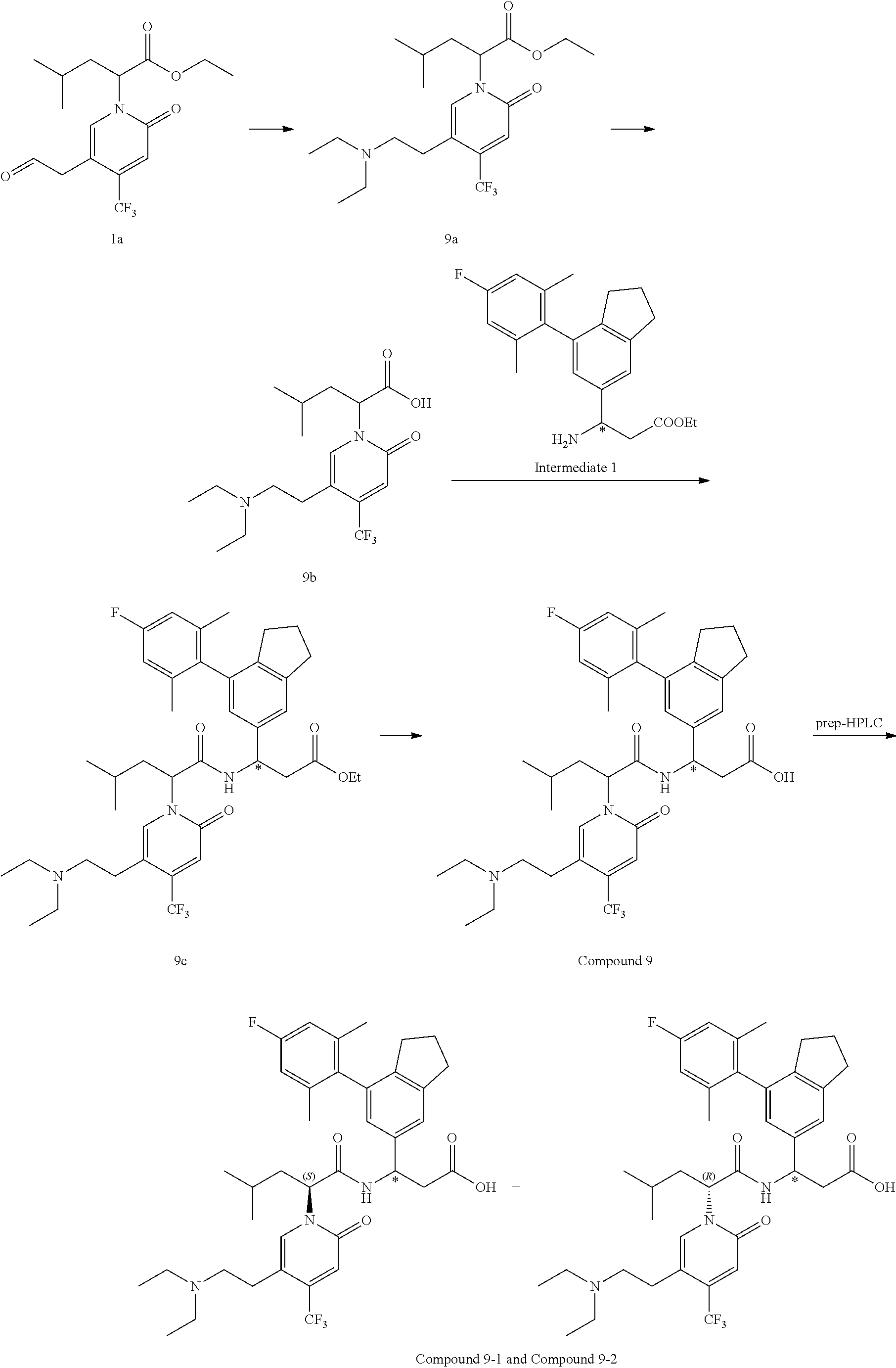

### Step 1: Synthesis of 9a

1a (3.0 g, 8.64 mmol) was dissolved in 30 mL of ultra-dry DCE, and diethylamine (0.95 g, 12.96 mmol) and AcOH (0.10 mg, 1.73 mmol) were successively added. The reaction was carried out for 1 h at room temperature, sodium triacetoxyborohydride (3.66 g, 17.28 mmol) was added and the reaction was continued for 16 h. Ethyl acetate (200 mL) and saturated NaHCO₃ aqueous solution (100 mL) were added for separation by extraction, and the aqueous phase was extracted again with ethyl acetate (100 mL×1). The ethyl acetate layers were combined, and the ethyl acetate layers were washed with saturated sodium chloride aqueous solution (100 mL×1), dried over anhydrous sodium sulfate, and concentrated under reduced pressure. The residue was purified by column chromatography to afford 9a (2.8 g, yield: 80.12%).

LC-Ms m/z (ESI): 405.3 [M+H]⁺

### Step 2: Synthesis of 9b

9a (2.8 g, 6.92 mmol) was dissolved in THF (30 mL) and water (10 mL), lithium hydroxide (331.55 mg, 13.85 mmol) was added, and the reaction was continued for 30 min. The reaction solution was purified by reverse phase column chromatography to afford 9b (1.2 g, yield: 46.05%).

LC-Ms m/z (ESI): 377.2 [M+H]⁺

### Step 3: Synthesis of 9c

Under nitrogen protection, intermediate 1 (300.0 mg, 0.84 mmol) was dissolved in dry DMF (5 mL), and HOBt (227.00 mg, 1.68 mmol), EDCI (322.06 mg, 1.68 mmol) and DIPEA (434.25 mg, 3.36 mmol) were successively added. At room temperature, the reaction mixture was stirred for 40 min, and then compound 9b (316.18 mg, 0.84 mmol) was added. The resulting mixture was reacted at room temperature overnight. Ethyl acetate (100 mL) was added and the resulting mixture was successively washed with water (40 mL×2) and saturated sodium chloride aqueous solution (40 mL×1), dried over anhydrous sodium sulfate and filtered and the filtrate was concentrated under reduced pressure and subjected to column chromatography on a silica gel column to afford 9c (400.0 mg, yield: 66.71%).

LC-Ms m/z (ESI): 714.4 [M+H]⁺

### Step 4: Synthesis of Compound 9-1 and Compound 9-2

Compound 9d (400.0 mg, 0.56 mmol) was dissolved in methanol (3 mL) and water (1 mL), lithium hydroxide monohydrate (27.0 mg, 1.12 mmol) was added, and the resulting mixture was reacted at room temperature for 30 min. PH was adjusted to 5-6 with 1 N hydrochloric acid and concentrated under reduced pressure to afford crude compound 9, which was separated and purified by prep-HPLC (Method 2) to afford the trifluoroacetate salt of compound 9-1 (76.0 mg, yield: 8.36%) and the trifluoroacetate salt of compound 9-2 (81.0 mg, yield: 6.27%).

### Trifluoroacetate Salt of Compound 9-1:

LC-Ms m/z (ESI): 686.2 [M+H]⁺

¹H NMR (400 MHz, CD₃OD) δ 7.91 (s, 1H), 7.19 (s, 1H), 6.82-6.73 (m, 4H), 5.74-5.66 (m, 1H), 5.35-5.27 (m, 1H), 3.21-3.06 (m, 6H), 3.00-2.85 (m, 4H), 2.73-2.63 (m, 2H), 2.43-2.35 (m, 2H), 2.02-1.92 (m, 4H), 1.89 (s, 3H), 1.82 (s, 3H), 1.48-1.36 (m, 1H), 1.26 (t, 6H), 0.99-0.90 (m, 6H).

### Trifluoroacetate Salt of Compound 9-2:

LC-Ms m/z (ESI): 686.2 [M+H]⁺

¹H NMR (400 MHz, CD₃OD) δ 7.86 (s, 1H), 7.22 (s, 1H), 6.90-6.77 (m, 4H), 5.66-5.57 (m, 1H), 5.41-5.33 (m, 1H), 3.30-3.17 (m, 6H), 3.03-2.90 (m, 4H), 2.68-2.53 (m, 2H), 2.48-2.38 (m, 2H), 2.07-1.89 (m, 9H), 1.78-1.67 (m, 1H), 1.43-1.34 (m, 1H), 1.29 (t, 6H), 0.92-0.82 (m, 6H).

### Example 10: Preparation of Compound 10

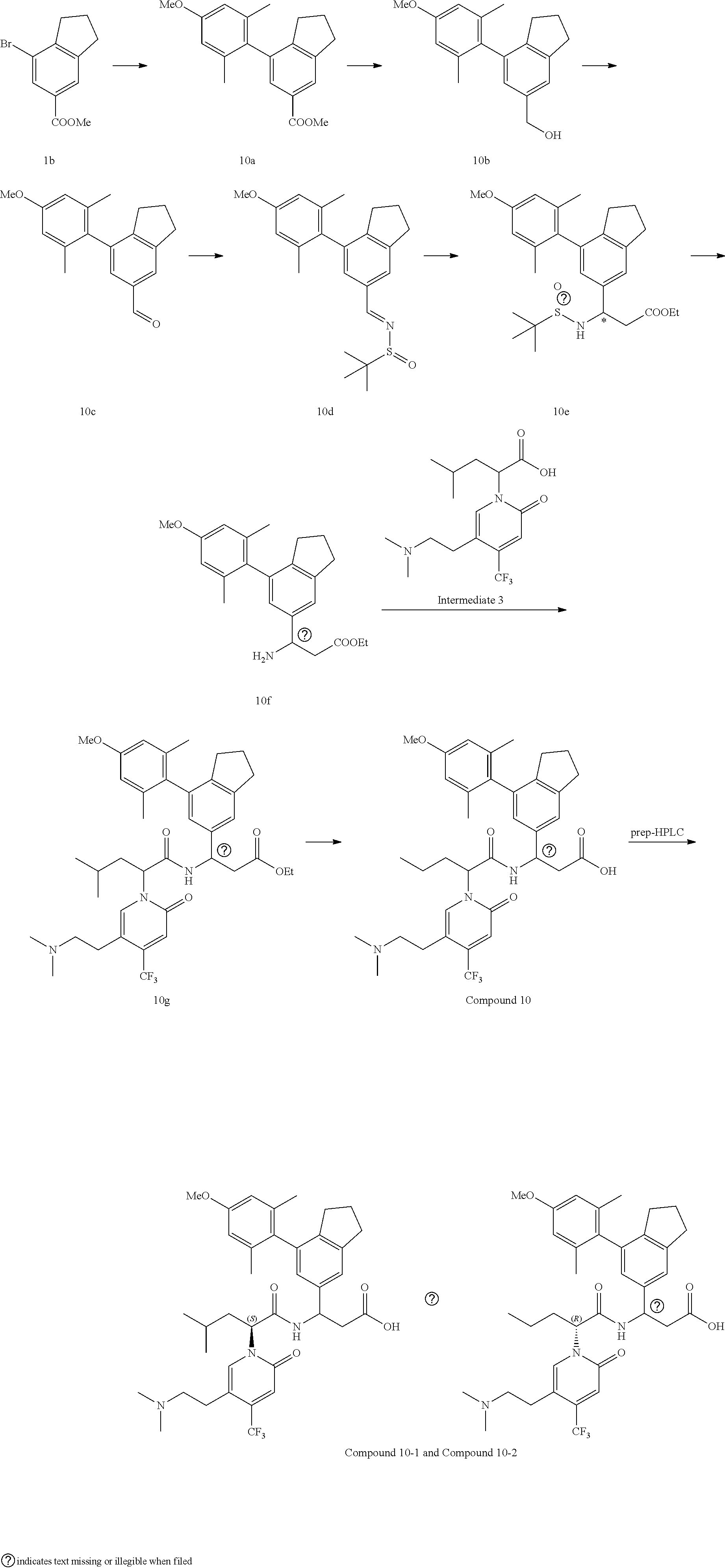

### Step 1: Synthesis of 10a

Under nitrogen protection, 1D (2.0 g, 7.84 mmol), 2.6-dimethyl-4-methoxy phenylboronic acid (2.82 g, 15.68 mmol) and cesium carbonate (7.66 g, 23.52 mmol) were added to 1,4-dioxane (30.0 mL) and water (3.0 mL), followed by Pd(PPh₃)₄ (0.29 g, 0.78 mmol), and the mixture was reacted at 100° C. in a sealed tube for 12 h. The reaction system was cooled to room temperature and then filtered through celite. Ethyl acetate (50 mL) was added and the mixture was successively washed with water (30 mL) and saturated sodium chloride aqueous solution (30 mL), dried over anhydrous sodium sulfate, filtered and concentrated under reduced pressure. The residue was subjected to flash chromatography on a silica gel column to afford 10a (1.6 g, yield: 65.75%).

### Step 2: Synthesis of 10b

Under nitrogen protection, 10a (1.6 g, 5.15 mmol) was dissolved in dry THF (10 mL) and at 0° C., lithium aluminum hydride (8 mL) was slowly added. The resulting mixture was reacted at room temperature for 1 h. 10% sodium sulfate aqueous solution (20 mL) was added and the mixture was filtered. The solvent was removed under reduced pressure and the residue was subjected to flash chromatography on a silica gel column to afford 10b (1.0 g, yield: 68.76%).

### Step 3: Synthesis of 10c

Under nitrogen protection, the compound 10b (1.0 g, 3.54 mmol) was dissolved in dry dichloromethane (10 mL), sodium bicarbonate (0.6 g, 7.08 mmol) and Dess-Martin periodinane (2.25 g, 5.31 mmol) were added at room temperature, and the resulting mixture was reacted at room temperature for 1 h. Saturated sodium thiosulfate solution (10 mL) and saturated sodium bicarbonate solution (10 mL) were added and the mixture was extracted with dichloromethane (50 mL×3), dried over anhydrous sodium sulfate and filtered. The solvent was removed under reduced pressure and the residue was subjected to column chromatography on a silica gel column to afford 10c (0.8 g, yield: 80.58%).

### Step 4: Synthesis of 10d

Under nitrogen protection, 10c (1.4 g, 4.99 mmol) and R-tert-butylsulfinamide (0.82 g, 5.99 mmol) were dissolved in THF (15 mL), tetraethyl titanate (1.71 g, 7.50 mmol) was slowly added, and the resulting mixture was reacted at 45° C. for 15 h and concentrated under reduced pressure to afford a crude, which was subjected to flash column chromatography on a silica gel column to afford 10d (1.9 g, yield: 99.27%).

### Step 5: Synthesis of 10e

Zinc powder (4.77 g, 72.94 mmol) was added to dry THF (5 mL), the mixture was subjected to nitrogen replacement three times, CuCl (1.55 g, 15.63 mmol) was added, and the resulting mixture was reacted at 60° C. for 2 h. The reaction mixture was cooled to room temperature, ethyl bromoacetate (4.35 g, 26.05 mmol) was slowly added, and the resulting mixture was reacted at 60° C. for 1 h and cooled to 0° C. A solution of 10d (2.0 g, 5.21 mmol) in THF (1 mL) was added and the mixture was stirred at 0° C. for 3 h. The reaction mixture was filtered through celite and saturated ammonium chloride solution (80 mL) was added. The resulting mixture was extracted with ethyl acetate (100 ml×3), washed with saturated sodium chloride aqueous solution (80 mL), dried over anhydrous sodium sulfate, filtered and concentrated under reduced pressure. The residue was subjected to flash column chromatography to afford 10e (2.0 g, yield: 81.39%).

### Step 6: Synthesis of 10f

10e (500.0 mg, 1.06 mmol) was dissolved in THF (5 mL), 4 N hydrogen chloride dioxane solution (5 mL) was added, and the resulting mixture was stirred at room temperature for 3 h. The reaction mixture was concentrated under reduced pressure to afford crude 10f.

LC-Ms m/z (ESI): 368.0 [M+H]⁺

### Step 7: Synthesis of 10g

Under nitrogen protection, intermediate 3 (189.6 mg, 0.54 mmol) was dissolved in dry DMF (1.5 mL), and HOBt (147.08 mg, 1.090 mmol), EDCI (168.98 mg, 1.09 mmol) and DIPEA (220.3 mg, 2.18 mmol) were successively added. At room temperature, the reaction mixture was stirred for 40 min, and then crude 10f (200.0 mg, 0.54 mmol) was added. The resulting mixture was reacted at room temperature overnight. Ethyl acetate (80 mL) was added and the resulting mixture was successively washed with water (20 mL×2) and saturated sodium chloride aqueous solution (20 mL×1), dried over anhydrous sodium sulfate and filtered and the filtrate was concentrated under reduced pressure and subjected to column chromatography on a silica gel column to afford 10g (180.0 mg, yield: 47.39%).

LC-Ms m/z (ESI): 698.3 [M+H]⁺

### Step 8: Synthesis of Compound 10-1 and Compound 10-2

10g (180.0 mg, 0.26 mmol) was dissolved in THF (3 mL) and water (1 mL), lithium hydroxide monohydrate (12.45 mg, 0.52 mmol) was added, and the resulting mixture was reacted at room temperature for 5 h. PH was adjusted to 5-6 with 1 N hydrochloric acid and concentrated under reduced pressure to afford crude compound 10, which was separated and purified by prep-HPLC (Method 2) to afford the trifluoroacetate salt of compound 10-1 (17 mg, yield: 9.76%) and the trifluoroacetate salt of compound 10-2 (15 mg, yield: 8.61%).

### Trifluoroacetate Salt of Compound 10-1:

LC-Ms m/z (ESI): 670.3 [M+H]⁺

¹H NMR (400 MHz, CD₃OD) δ 7.92 (s, 1H), 7.14 (s, 1H), 6.80 (s, 1H), 6.74 (s, 1H), 6.67-6.56 (m, 2H), 5.79-5.70 (m, 1H), 5.37-5.27 (m, 1H), 3.77 (s, 3H), 3.29-3.20 (m, 2H), 3.05-2.88 (m, 10H), 2.87-2.76 (m, 2H), 2.46-2.35 (m, 2H), 2.04-1.89 (m, 4H), 1.85 (s, 3H), 1.78 (s, 3H), 1.51-1.39 (m, 1H), 1.02-0.91 (m, 6H).

### Trifluoroacetate Salt of Compound 10-2:

LC-Ms m/z (ESI): 670.3 [M+H]⁺

¹H NMR (400 MHz, CD₃OD) δ 7.92 (s, 1H), 7.20 (s, 1H), 6.88 (s, 1H), 6.82 (s, 1H), 6.66 (s, 2H), 5.78-5.69 (m, 1H), 5.37-5.24 (m, 1H), 3.78 (s, 3H), 3.35-3.24 (m, 2H), 3.05-2.92 (m, 10H), 2.89-2.76 (m, 2H), 2.50-2.42 (m, 2H), 2.09-1.96 (m, 2H), 1.91 (s, 6H), 1.86-1.77 (m, 2H), 1.39-1.25 (m, 1H), 0.95-0.81 (m, 6H).

### Example 11: Preparation of Compound 11

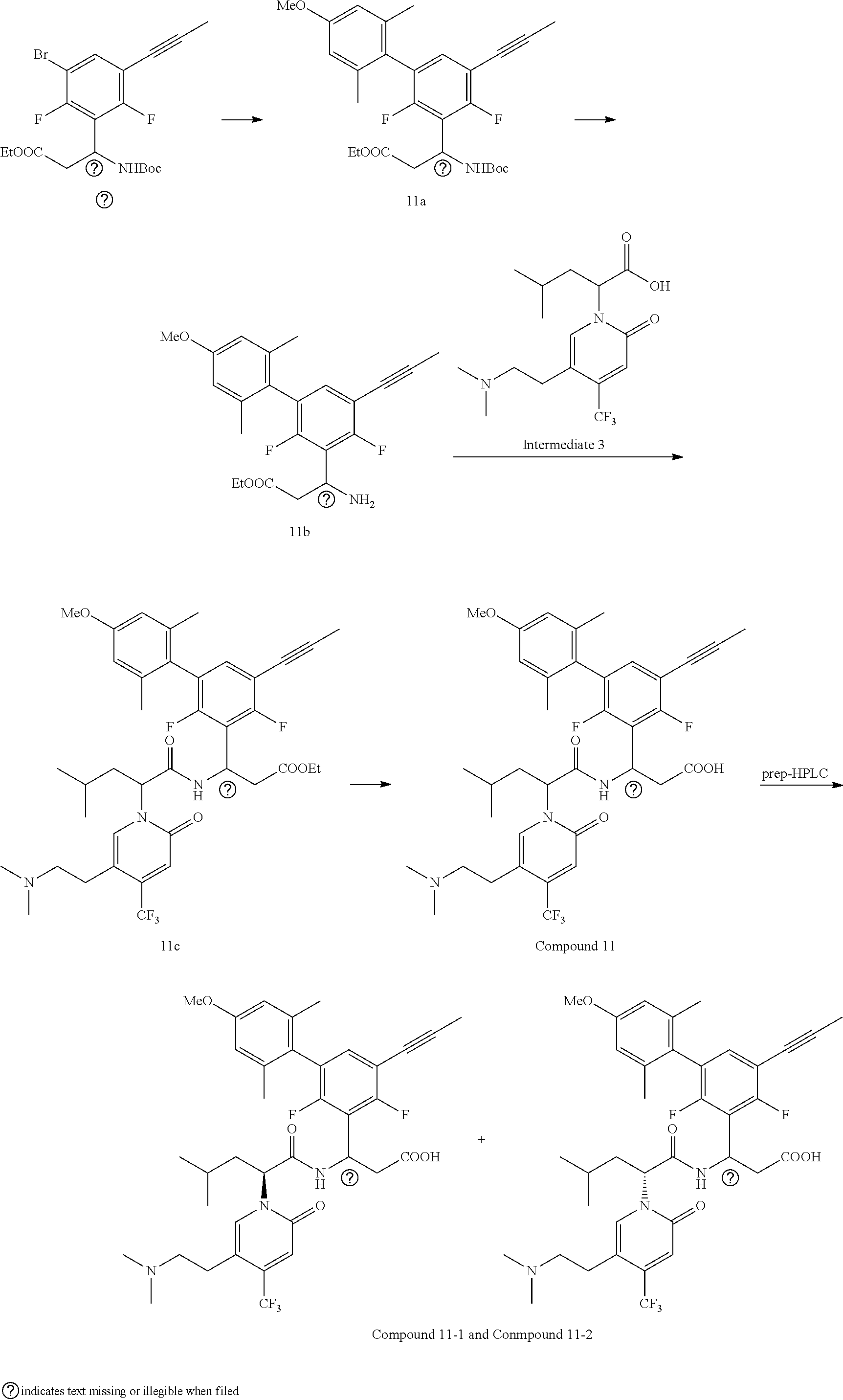

### Step 1: Synthesis of 11a

Under nitrogen protection, 2g (300.0 mg, 0.67 mmol), 2.6-dimethyl-4-methoxy phenylboronic acid (242.0 mg, 1.34 mmol) and cesium carbonate (0.65 g, 2.01 mmol) were added to 1,4-dioxane (5.0 mL) and water (0.5 mL), followed by Pd(PPh₃)₄ (77.0 mg, 0.067 mmol), and the mixture was reacted at 100° C. in a sealed tube for 12 h. The reaction system was cooled to room temperature and then filtered through celite. Ethyl acetate (50 mL) was added and the mixture was successively washed with water (30 mL) and saturated sodium chloride aqueous solution (30 mL), dried over anhydrous sodium sulfate, filtered and concentrated under reduced pressure. The residue was subjected to flash chromatography on a silica gel column to afford 11a (300.0 mg, yield: 92.22%).

### Step 2: Synthesis of 11b

11a (300.0 mg, 0.60 mmol) was dissolved in acetonitrile (3 mL), hydrogen chloride-1,4-dioxane solution (2 mL) was added, and the resulting mixture was stirred at room temperature for 30 min. The solvent was removed by drying under reduced pressure to afford crude 11b.

LC-Ms m/z (ESI): 402.7 [M+H]⁺

### Step 3: Synthesis of 11c

Under nitrogen protection, intermediate 3 (174.18 mg, 0.50 mmol) was dissolved in dry DMF (5 mL), and HOBt (135.12 mg, 1.00 mmol), EDCI (191.70 mg, 1.00 mmol) and DIPEA (258.48 mg, 2.00 mmol) were successively added. At room temperature, the reaction mixture was stirred for 40 min, and then crude 11b (200.0 mg, 0.50 mmol) was added. The resulting mixture was reacted at room temperature overnight. Ethyl acetate (80 mL) was added and the resulting mixture was successively washed with water (20 mL×2) and saturated sodium chloride aqueous solution (20 mL×1), dried over anhydrous sodium sulfate and filtered and the filtrate was concentrated under reduced pressure and subjected to column chromatography on a silica gel column to afford 11c (300.0 mg, yield: 81.99%).

LC-Ms m/z (ESI): 732.3 [M+H]⁺

### Step 4: Synthesis of Compound 11-1 and Compound 11-2

11c (300.0 mg, 0.41 mmol) was dissolved in THF (3 mL) and water (1 mL), lithium hydroxide monohydrate (19.46 mg, 0.82 mmol) was added, and the resulting mixture was reacted at room temperature for 5 h. PH was adjusted to 5-6 with 1 N hydrochloric acid, and the resulting solution was concentrated under reduced pressure to afford crude compound 11, which was separated and purified by prep-HPLC (method 2) to afford the trifluoroacetate salt of compound 11-1 (23 mg, Rt=4.024 min, yield: 7.97%) and the trifluoroacetate salt of compound 11-2 (27 mg, Rt=4.165 min, yield: 9.36%).

### Trifluoroacetate Salt of Compound 11-1:

LC-Ms m/z (ESI): 704.3 [M+H]⁺

¹H NMR (400 MHz, CD₃OD) δ 7.87 (s, 1H), 7.03 (t, 1H), 6.84 (s, 1H), 6.69-6.63 (m, 2H), 5.76-5.62 (m, 2H), 3.78 (s, 3H), 3.24-3.14 (m, 2H), 3.03-2.89 (m, 3H), 2.85 (s, 6H), 2.81-2.70 (m, 1H), 2.03 (s, 3H), 2.01-1.92 (m, 5H), 1.87 (s, 3H), 1.46-1.32 (m, 1H), 0.99-0.88 (m, 6H).

### Trifluoroacetate Salt of Compound 11-1:

LC-Ms m/z (ESI): 704.3 [M+H]⁺

¹H NMR (400 MHz, CD₃OD) δ 7.83 (s, 1H), 7.08 (t, 1H), 6.90 (s, 1H), 6.69 (s, 2H), 5.81-5.74 (m, 1H), 5.70-5.61 (m, 1H), 3.79 (s, 3H), 3.30-3.17 (m, 2H), 3.05-2.95 (m, 2H), 2.91-2.78 (m, 7H), 2.69-2.58 (m, 1H), 2.06 (s, 3H), 2.02-1.96 (m, 6H), 1.94-1.82 (m, 1H), 1.81-1.70 (m, 1H), 1.41-1.25 (m, 1H), 0.94-0.82 (m, 6H).

### Example 12: Synthesis of Compound 12

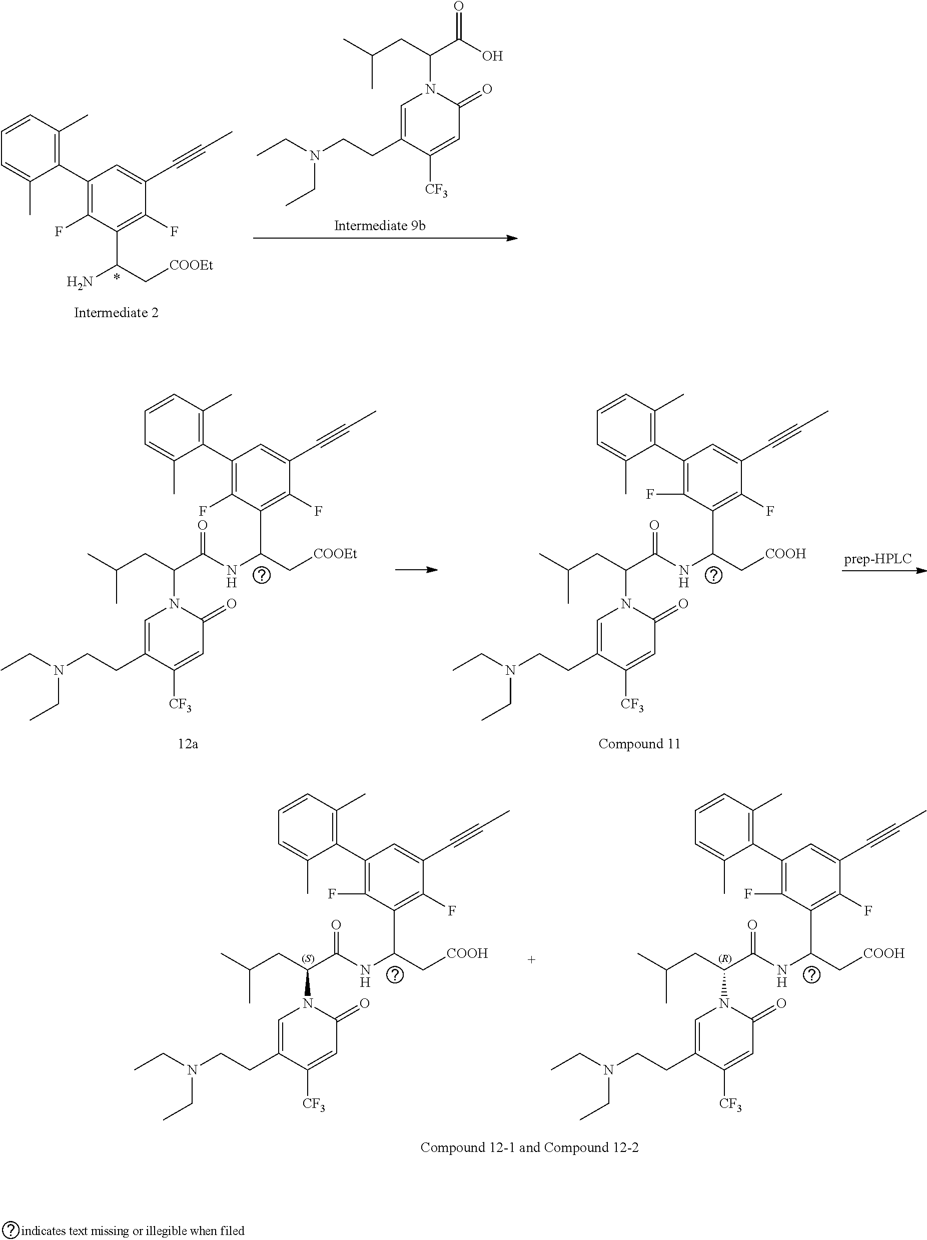

Referring to the preparation method of compound 9, crude compound 12 was obtained, which was separated and purified by prep-HPLC (method 2) to afford the trifluoroacetate salt of compound 12-1 (11 mg, Rt=4.191 min) and the trifluoroacetate salt of compound 12-2 (17 mg, Rt=4.332 min).

### Trifluoroacetate Salt of Compound 12-1:

LC-Ms m/z (ESI): 702.3 [M+H]⁺

¹H NMR (400 MHz, CD₃OD) δ 7.88 (s, 1H), 7.19-7.11 (m, 1H), 7.10-6.98 (m, 3H), 6.84 (s, 1H), 5.77-5.64 (m, 2H), 3.22-3.10 (m, 6H), 3.03-2.87 (m, 3H), 2.72-2.63 (m, 1H), 2.03 (s, 3H), 2.00-1.93 (m, 5H), 1.89 (s, 3H), 1.46-1.34 (m, 1H), 1.27 (t, 6H), 0.98-0.87 (m, 6H).

### Trifluoroacetate Salt of Compound 12-2:

LC-Ms m/z (ESI): 702.3 [M+H]⁺

¹H NMR (400 MHz, CD₃OD) δ 7.89-7.83 (m, 1H), 7.20-7.14 (m, 1H), 7.13-7.02 (m, 3H), 6.89 (s, 1H), 5.80-5.72 (m, 1H), 5.69-5.62 (m, 1H), 3.29-3.18 (m, 6H), 3.01-2.92 (m, 2H), 2.90-2.80 (m, 1H), 2.68-2.57 (m, 1H), 2.06 (s, 3H), 2.03-1.98 (m, 6H), 1.94-1.83 (m, 1H), 1.78-1.67 (m, 1H), 1.39-1.24 (m, 7H), 0.93-0.82 (m, 6H).

### Example 13: Preparation of Compound 13

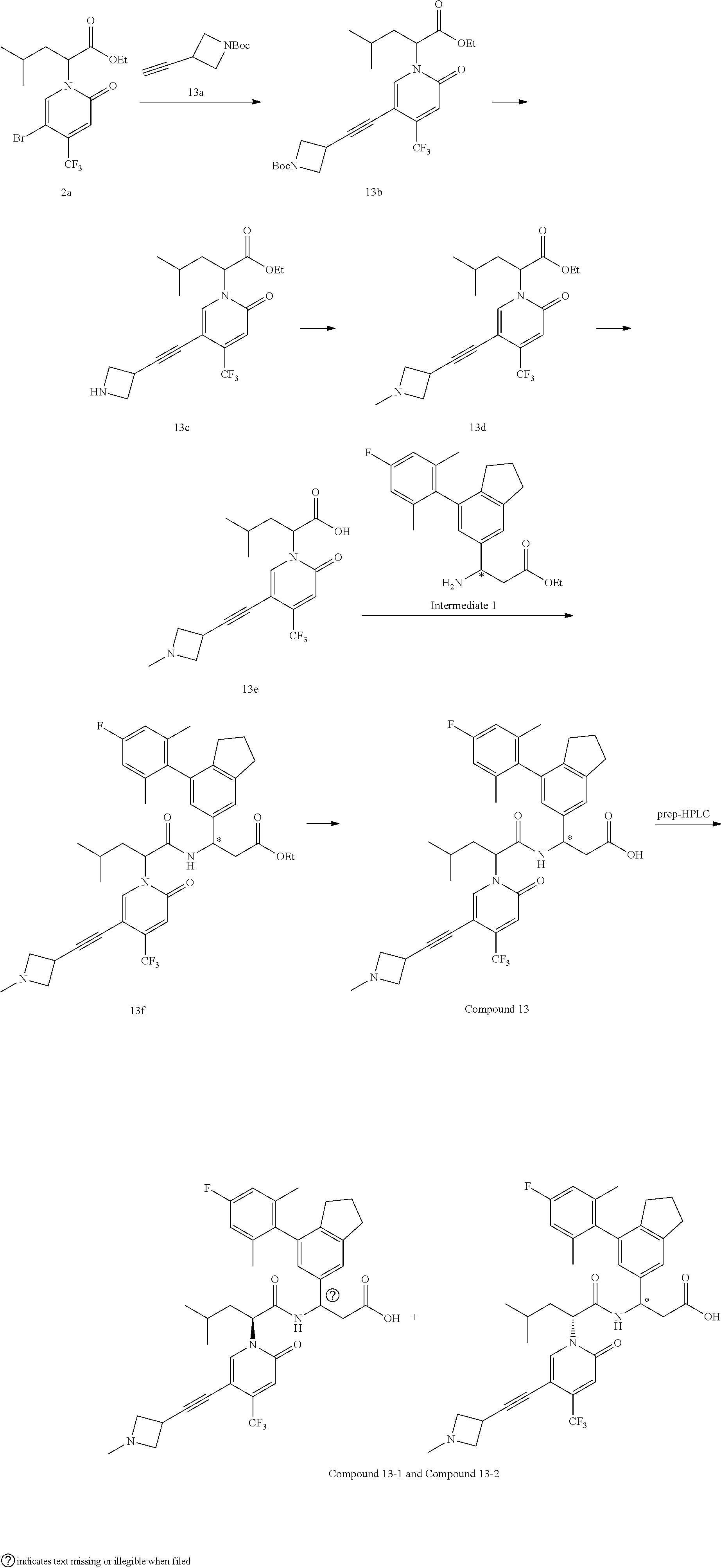

### Step 1: Preparation of 13b

2a (2.0 g, 5.2 mmol), 13a (1.9 g, 10.4 mmol), triethylamine (1.4 mL, 10.4 mmol), cuprous iodide (4.0 g, 20.8 mmol), anhydrous lithium chloride (1.76 g, 41.6 mmol) and (PPh₃)₂PdCl₂ (730 mg, 1.04 mmol) were placed in a sealed tube, and DMF (30 mL) and water (3 mL) were added. The mixture was stirred at 80° C. under nitrogen protection for 3 h. The reaction solution was cooled to room temperature. 100 mL of ethyl acetate was added for dilution, and the mixed solution was successively washed with water (50 mL×3) and saturated sodium chloride aqueous solution (50 mL×1). The organic layer was dried over anhydrous sodium sulfate and filtered, and the filtrate was concentrated under reduced pressure, and the residue was subjected to column chromatography to afford 13b (1.86 g, yield: 74%).

LCMS m/z=485.2 [M+H]⁺

### Step 2: Preparation of 13c

13b (1.86 g, 3.84 mmol) was dissolved in 20 mL of dichloromethane, and 5 mL of trifluoroacetic acid was added. The mixture was stirred at room temperature for 3 h. The reaction mixture was concentrated under reduced pressure to afford crude 13c (1.8 g).

LCMS m/z=385.2 [M+H]⁺

### Step 3: Preparation of 13d

The crude 13c (1.8 g), aqueous carbaldehyde (5 mL) and glacial acetic acid (230 mg, 3.84 mmol) were dissolved in methanol (20 mL), stirred at room temperature under nitrogen protection for 30 min, and sodium triacetoxyborohydride (1.62 g, 7.68 mmol) was added. The stirring was continued overnight at room temperature. The solvent was removed by concentration under reduced pressure, 30 mL of water was added, and the aqueous phase was extracted with ethyl acetate (50 mL×2), and the organic phases were combined. The organic phase was washed with saturated sodium chloride aqueous solution (30 mL×1), the organic layer was dried over anhydrous sodium sulfate and filtered, and the filtrate was concentrated under reduced pressure. The residue was subjected to column chromatography to afford 13d (0.91 g, two-step yield: 60%).

LCMS m/z=399.2 [M+H]⁺

### Step 4: Preparation of 13e

13d (0.91 g, 2.29 mmol) was dissolved in ethanol (15 mL) and water (1.5 mL), lithium hydroxide monohydrate (288 mg, 6.86 mmol) was added, and the reaction was carried out at room temperature for 5 h. The solvent was removed by concentration under reduced pressure, 20 mL of water was added to the residue, and the pH was adjusted to around 2-3 with 1N hydrochloric acid. The aqueous phase was extracted with ethyl acetate (50 mL×2), and the organic phases are combined. The organic phase was washed with saturated sodium chloride aqueous solution (30 mL×1), the organic layer was dried over anhydrous sodium sulfate and filtered, and the filtrate was concentrated under reduced pressure. The residue was subjected to reverse phase (0.1% trifluoroacetic acid) preparative chromatography to afford 13e (200 mg, yield: 24%).

LCMS m/z=371.2 [M+H]⁺;

### Step 5: Preparation of 13f

13e (0.160 g, 0.43 mmol) and intermediate 1 (0.15 g, 0.43 mmol) were dissolved in 5 mL DMF. HOBT (0.12 g, 0.86 mmol), DIPEA (0.22 g, 1.72 mmol) and EDCI (0.16 g, 0.86 mmol) were added, and stirred at room temperature for 2 h. After the reaction was complete, 30 ml of ethyl acetate was added, and the reaction solution was washed with water twice (20 mL×2), dried over anhydrous sodium sulfate, concentrated under reduced pressure, and subjected to column chromatography to afford 13f (0.13 g, yield: 42.71%).

LCMS m/z=708.9 [M+H]⁺;

### Step 6: Preparation of Compounds 13-1 and 13-2

13f (0.13 g, 0.18 mmol) was dissolved in THF (3 mL) and water (1 mL), lithium hydroxide monohydrate (23 mg, 0.55 mmol) was added, and the resulting mixture was reacted at room temperature for 5 h. PH was adjusted to 5-6 with 1 N hydrochloric acid, and the resulting solution was concentrated under reduced pressure to afford crude compound 13, which was separated and purified by prep-HPLC (method 1) to afford compound 13-1 (0.55 mg, Rt=4.271 min) and compound 13-2 (0.6 mg, Rt=4.358 min).

### Compound 13-1:

LC-Ms m/z (ESI): 680.2 [M+H]⁺

¹H NMR (400 MHz, Methanol-d₄) δ 8.03 (s, 1H), 7.15 (s, 1H), 6.84-6.70 (m, 4H), 5.72-5.65 (m, 1H), 5.31 (t, 1H), 3.93-3.81 (m, 1H), 3.58-3.48 (m, 2H), 3.15-3.12 (m, 1H), 2.95-2.85 (m, 2H), 2.70 (d, 2H), 2.52 (s, 3H), 2.42-2.35 (m, 2H), 2.03-1.91 (m, 4H), 1.89 (s, 3H), 1.79 (s, 3H), 1.48-1.29 (m, 2H), 1.00-0.90 (m, 6H)

### Compound 13-2:

LC-Ms m/z (ESI): 680.2[M+H]⁺

¹H NMR (400 MHz, Methanol-d₄) δ 8.08 (s, 1H), 7.23 (s, 1H), 6.86-6.78 (m, 4H), 5.69-5.58 (m, 1H), 5.40-5.29 (m, 1H), 3.96-3.87 (m, 1H), 3.70-3.47 (m, 2H), 3.14-3.10 (m, 1H), 2.99-2.91 (m, 2H), 2.72-2.64 (m, 2H), 2.55 (s, 3H), 2.48-2.39 (m, 2H), 2.22-2.14 (m, 1H), 2.06-1.98 (m, 3H), 1.96-1.90 (m, 6H), 1.77-1.69 (m, 1H), 1.66-1.55 (m, 1H), 0.91-0.83 (m, 6H).

### Example 14: Preparation of Compound 14

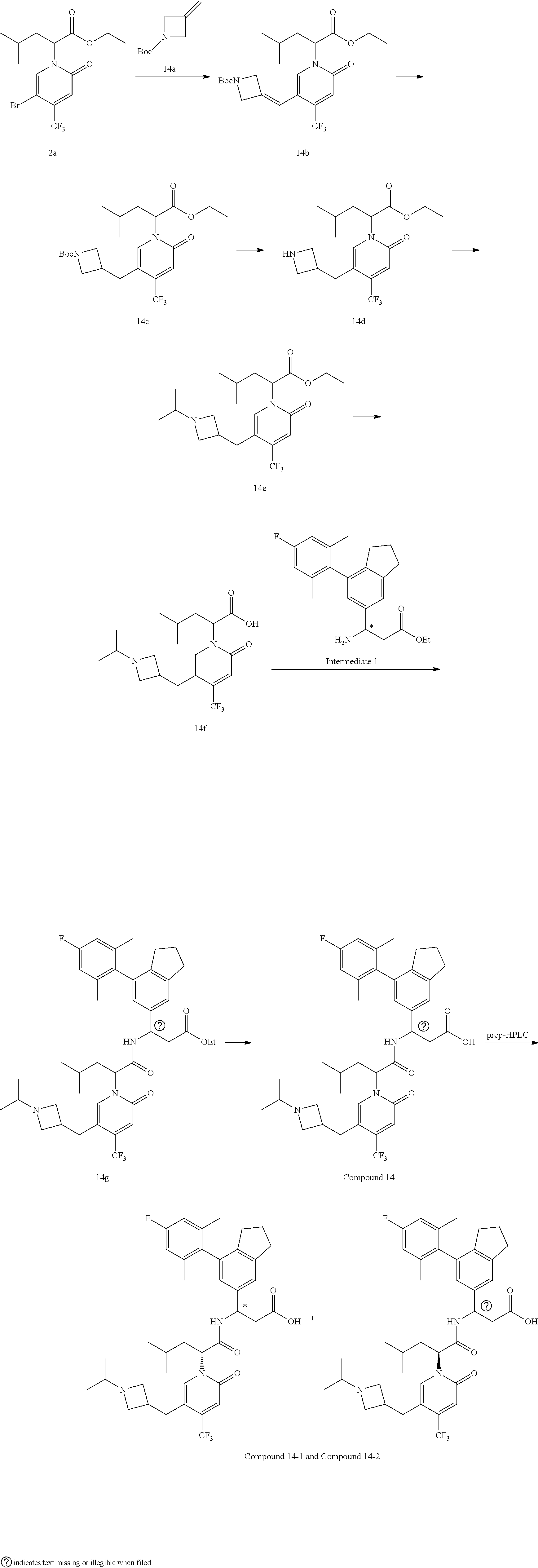

### Step 1: Synthesis of 14b

Under nitrogen protection, 2a (20.0 g, 52.04 mmol) was dissolved in DMF (60 mL). 14a (10.56 g, 62.44 mmol), palladium acetate (3.32 g, 10.4 mmol), tris(2-methylphenyl)phosphine (6.32 g, 20.8 mmol) and triethylamine (15.8 g, 156.12 mmol) were successively added, and the resulting mixture was reacted at 100° C. for 24 h. Extraction was performed by addition of ethyl acetate (180 mL) and the resulting mixture was washed with water (50 mL×2) and saturated sodium chloride aqueous solution (50 mL×1), dried over anhydrous sodium sulfate and filtered and the filtrate was concentrated under reduced pressure and subjected to column chromatography on a silica gel column to afford 14b (4.0 g, yield: 16.27%).

LC-Ms m/z (ESI): 473.5 [M+H]⁺

### Step 2: Synthesis of 14c

14b (4.0 g, 8.46 mmol) was dissolved in methanol (10 mL), Pd/C (2.0 g) was added, and the mixed solution was reacted under hydrogen atmosphere at room temperature for 0.5 h. The solid was removed by filtration and the filtrate was concentrated and purified by column chromatography to afford 14c (3.6 g, yield: 89.68%).

LC-Ms m/z (ESI): 475.5 [M+H]⁺

### Step 3: Synthesis of 14d

14c (3.6 g, 7.59 mmol) was dissolved in dichloromethane (5 mL), hydrogen chloride-1,4-dioxane (3 mL) was added, and the reaction was carried out for 1 h. The solvent was removed under reduced pressure to afford the hydrochloride salt of crude 14d.

### Step 4: Synthesis of 14e

The hydrochloride salt of 14d (1.3 g, 3.47 mmol) was dissolved in ultra-dry 1,2-dichloroethane (30 mL), acetone (400 mg, 6.87 mmol) and glacial acetic acid (21.0 mg, 0.35 mmol) were successively added, and the reaction was carried out at 60° C. for 2 h. The reaction liquid was cooled to room temperature, and sodium triacetoxyborohydride (1.84 g, 8.68 mmol) was then added. The resulting mixture was reacted at room temperature overnight. Extraction was performed by addition of ethyl acetate (20 mL) and the resulting mixture was washed with water (30 mL×2) and saturated sodium chloride aqueous solution (30 mL×1), dried over anhydrous sodium sulfate and filtered and the filtrate was concentrated under reduced pressure and subjected to column chromatography on a silica gel column to afford 14e (1.0 g, yield: 69.20%).

LC-Ms m/z (ESI): 417.4 [M+H]⁺

### Step 5: Synthesis of 14f

14e (1.0 g, 2.40 mmol) was dissolved in THF (9 mL) and water (3 mL), lithium hydroxide (170 mg, 7.10 mmol) was added, and the reaction was carried out at room temperature for 2 h. The reaction solution was purified by reverse phase column chromatography to afford 14f (800 mg, yield: 85.82%).

LC-Ms m/z (ESI): 389.4 [M+H]⁺

### Step 6: Synthesis of 14g

Under nitrogen protection, intermediate 1 (250.0 mg, 0.70 mmol) was dissolved in dry DMF (1.5 mL), and HOBt (190 mg, 1.41 mmol), EDCI (270 mg, 1.41 mmol) and DIPEA (270 mg, 2.09 mmol) were successively added. At room temperature, the reaction mixture was stirred for 40 min, and then 14f (270 mg, 0.70 mmol) was added. The resulting mixture was reacted at room temperature overnight. Ethyl acetate (80 mL) was added and the resulting mixture was successively washed with water (20 mL×2) and saturated sodium chloride aqueous solution (20 mL×1), dried over anhydrous sodium sulfate and filtered and the filtrate was concentrated under reduced pressure and subjected to column chromatography on a silica gel column to afford 14g (368 mg, yield: 72.43%).

LC-Ms m/z (ESI): 726.8 [M+H]⁺

### Step 7: Synthesis of Compound 14-1 and Compound 14-2

Compound 14g (368 mg, 0.51 mmol) was dissolved in methanol (1.5 mL) and water (0.5 mL), lithium hydroxide monohydrate (31 mg, 1.27 mmol) was added, and the resulting mixture was reacted at room temperature for 30 min. PH was adjusted to 5-6 with 1 N hydrochloric acid, and the resulting solution was concentrated under reduced pressure to afford crude compound 14, which was separated and purified by prep-HPLC (method 2) to afford the trifluoroacetate salt of compound 14-1 (20 mg, Rt=4.295 min, yield: 5.62%) and the trifluoroacetate salt of compound 14-2 (25 mg, Rt=4.378 min, yield: 7.02%).

### Trifluoroacetate Salt of Compound 14-1:

LC-Ms m/z (ESI): 698.8 [M+H]⁺

¹H NMR (400 MHz, CD₃OD) δ 7.90-7.68 (m, 1H), 7.20 (s, 1H), 6.88-6.73 (m, 4H), 5.84-5.75 (m, 1H), 5.39-5.27 (m, 1H), 4.32-3.83 (m, 4H), 3.52-3.34 (m, 1H), 3.20-2.81 (m, 7H), 2.49-2.34 (m, 2H), 2.10-1.92 (m, 4H), 1.90 (m, 3H), 1.83 (s, 3H), 1.52-1.35 (m, 1H), 1.30-1.11 (m, 6H), 1.05-0.93 (m, 6H).

### Trifluoroacetate Salt of Compound 14-2:

LC-Ms m/z (ESI): 698.8 [M+H]⁺

¹H NMR (400 MHz, CD₃OD) δ 7.91-7.76 (m, 1H), 7.25 (s, 1H), 6.92-6.78 (m, 4H), 5.84-5.73 (m, 1H), 5.39-5.26 (m, 1H), 4.34-3.85 (m, 4H), 3.53-3.35 (m, 1H), 3.24-2.95 (m, 4H), 2.94-2.77 (m, 3H), 2.53-2.43 (m, 2H), 2.12-2.00 (m, 2H), 1.96 (s, 6H), 1.91-1.78 (m, 2H), 1.40-1.20 (m, 7H), 0.96-0.85 (m, 6H).

### Example 15: Preparation of Compound 15

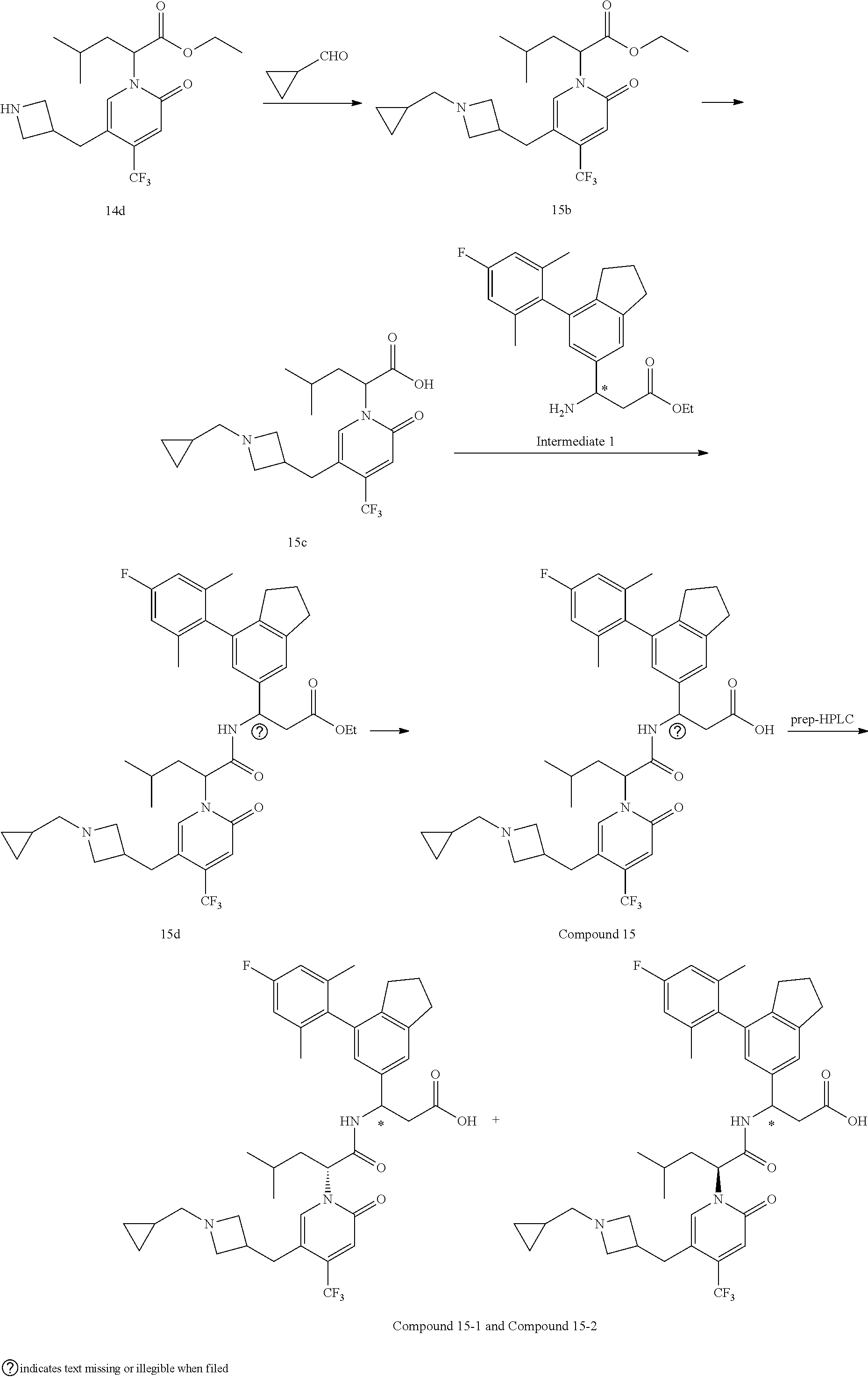

Referring to the preparation method of compound 14, crude compound 15 was obtained, which was separated and purified by prep-HPLC (method 2) to afford the trifluoroacetate salt of compound 15-1 (15 mg, Rt=4.354 min) and the trifluoroacetate salt of compound 15-2 (17 mg, Rt=4.455 min).

### Trifluoroacetate Salt of Compound 15-1:

LC-Ms m/z (ESI): 710.8 [M+H]⁺

¹H NMR (400 MHz, CD₃OD) δ 7.89-7.72 (s, 1H), 7.20 (s, 1H), 6.89-6.72 (m, 4H), 5.84-5.75 (m, 1H), 5.38-5.28 (m, 1H), 4.36-3.84 (m, 4H), 3.27-2.79 (m, 9H), 2.48-2.37 (m, 2H), 2.09-1.93 (m, 4H), 1.92-1.81 (m, 6H), 1.51-1.38 (m, 1H), 1.07-0.94 (m, 7H), 0.74-0.63 (m, 2H), 0.44-0.34 (m, 2H).

### Trifluoroacetate Salt of Compound 15-2:

LC-Ms m/z (ESI): 710.8 [M+H]⁺

¹H NMR (400 MHz, CD₃OD) δ 7.92-7.74 (s, 1H), 7.25 (s, 1H), 6.93-6.77 (m, 4H), 5.83-5.73 (m, 1H), 5.38-5.28 (m, 1H), 4.36-3.86 (m, 4H), 3.30-2.76 (m, 9H), 2.52-2.43 (m, 2H), 2.12-2.01 (m, 2H), 1.96 (s, 6H), 1.93-1.74 (m, 2H), 1.41-1.27 (m, 1H), 1.11-0.84 (m, 7H), 0.76-0.61 (m, 2H), 0.47-0.38 (m, 2H).

### Example 16: Preparation of Compound 16

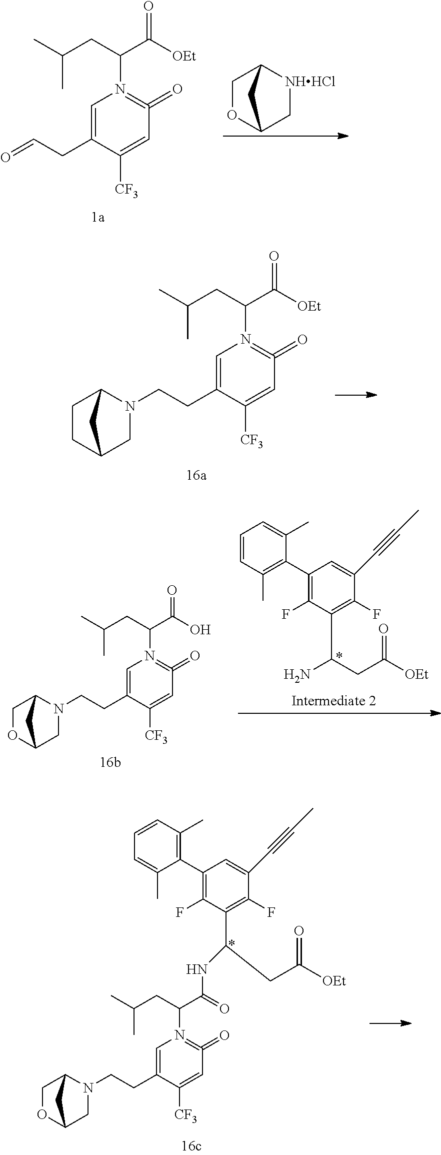

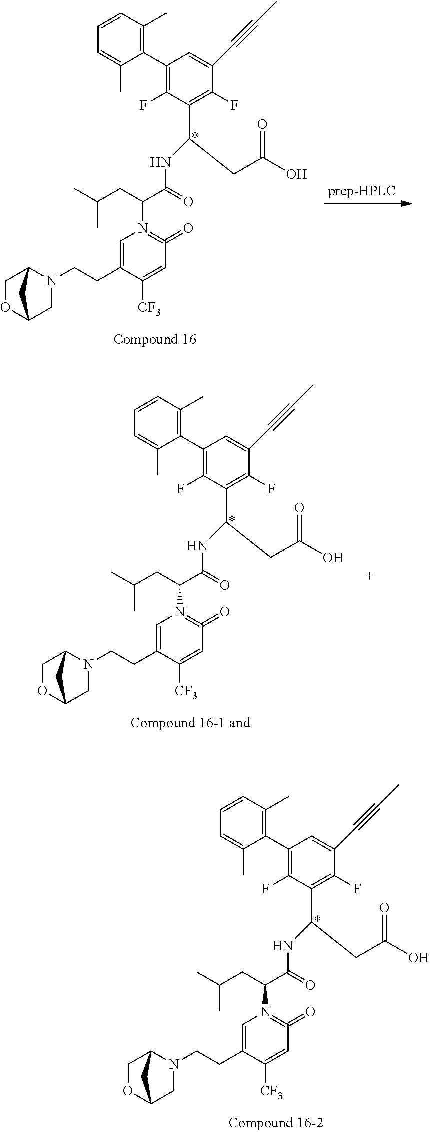

Referring to the preparation method of compound 3, crude compound 16 was obtained, which was separated and purified by prep-HPLC (method 2) to afford the trifluoroacetate salt of compound 16-1 (15 mg, Rt=4.077 min) and the trifluoroacetate salt of compound 16-2 (16 mg, Rt=4.178 min).

### Trifluoroacetate Salt of Compound 16-1:

LC-Ms m/z (ESI): 728.7 [M+H]⁺

¹H NMR (400 MHz, CD₃OD) δ 7.92 (s, 1H), 7.23-7.15 (m, 1H), 7.14-7.03 (m, 3H), 6.86 (s, 1H), 5.87-5.76 (m, 1H), 5.75-5.64 (m, 1H), 4.77-4.68 (m, 1H), 4.61-4.47 (m, 1H), 4.23-4.02 (m, 1H), 3.97-3.84 (m, 1H), 3.77-3.35 (m, 4H), 3.19-2.86 (m, 4H), 2.38-2.18 (m, 2H), 2.12-1.84 (m, 11H), 1.50-1.35 (m, 1H), 1.05-0.90 (m, 6H).

### Trifluoroacetate Salt of Compound 16-2:

LC-Ms m/z (ESI): 728.7 [M+H]⁺

¹H NMR (400 MHz, CD₃OD) δ 7.95 (s, 1H), 7.25-7.07 (m, 4H), 6.92 (s, 1H), 5.87-5.78 (m, 1H), 5.75-5.66 (m, 1H), 4.77-4.70 (m, 1H), 4.62-4.51 (m, 1H), 4.26-4.04 (m, 1H), 3.94-3.85 (m, 1H), 3.75-3.36 (m, 4H), 3.14-2.89 (m, 4H), 2.39-2.18 (m, 2H), 2.12-1.98 (m, 9H), 1.97-1.73 (m, 2H), 1.41-1.25 (m, 1H), 0.94-0.85 (m, 6H).

### Example 17: Preparation of Compound 17

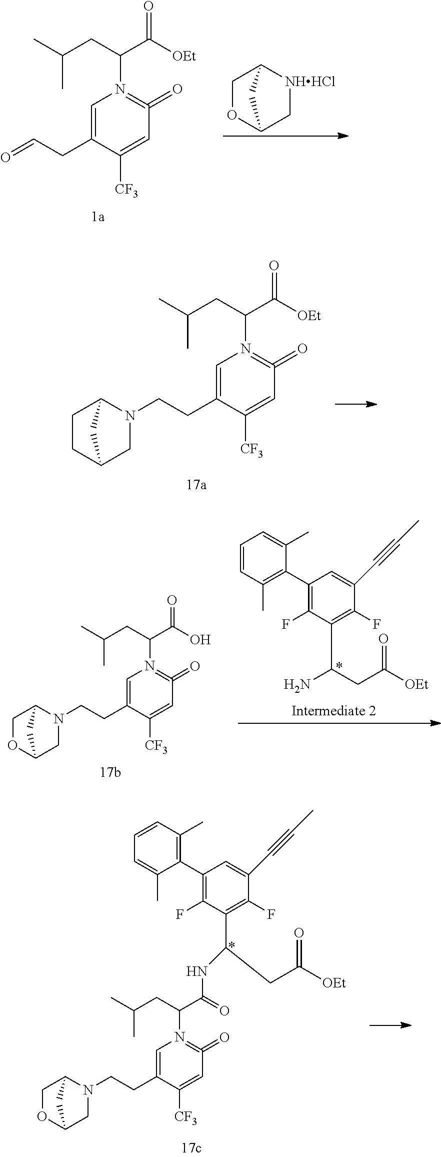

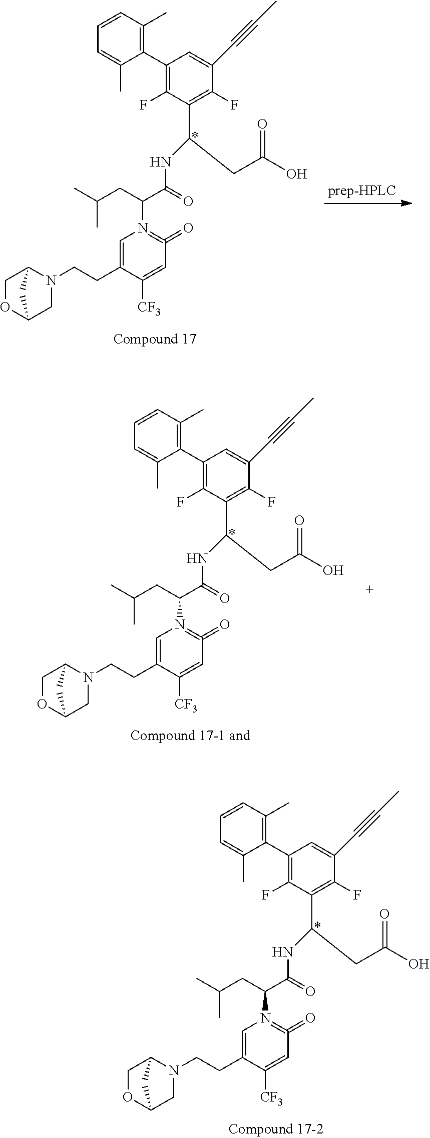

Referring to the preparation method of compound 3, crude compound 17 was obtained, which was separated and purified by prep-HPLC (method 2) to afford the trifluoroacetate salt of compound 17-1 (17 mg, Rt=4.035 min) and the trifluoroacetate salt of compound 17-2 (18 mg, Rt=4.118 min).

### Trifluoroacetate Salt of Compound 17-1:

LC-Ms m/z (ESI): 728.7 [M+H]⁺

¹H NMR (400 MHz, CD₃OD) δ 8.01-7.80 (s, 1H), 7.23-7.15 (m, 1H), 7.14-7.03 (m, 3H), 6.86 (s, 1H), 5.88-5.78 (m, 1H), 5.76-5.64 (m, 1H), 4.79-4.65 (m, 1H), 4.62-4.46 (m, 1H), 4.41-3.97 (m, 1H), 3.96-3.64 (m, 2H), 3.60-3.40 (m, 2H), 3.32-3.22 (m, 1H), 3.19-2.85 (m, 4H), 2.45-2.13 (m, 2H), 2.11-1.85 (m, 11H), 1.50-1.37 (m, 1H), 1.04-0.90 (m, 6H).

### Trifluoroacetate Salt of Compound 17-2:

LC-Ms m/z (ESI): 728.7 [M+H]⁺

¹H NMR (400 MHz, CD₃OD) δ 7.93 (s, 1H), 7.25-7.06 (m, 4H), 6.92 (s, 1H), 5.88-5.77 (m, 1H), 5.75-5.64 (m, 1H), 4.79-4.68 (m, 1H), 4.62-4.48 (m, 1H), 4.24-3.98 (m, 1H), 3.97-3.84 (m, 1H), 3.82-3.62 (m, 1H), 3.62-3.37 (m, 3H), 3.15-2.89 (m, 4H), 2.44-2.16 (m, 2H), 2.09 (s, 3H), 2.04 (s, 6H), 1.95-1.71 (m, 2H), 1.38-1.26 (m, 1H), 0.98-0.84 (m, 6H).

### Example 18: Preparation of Compound 18

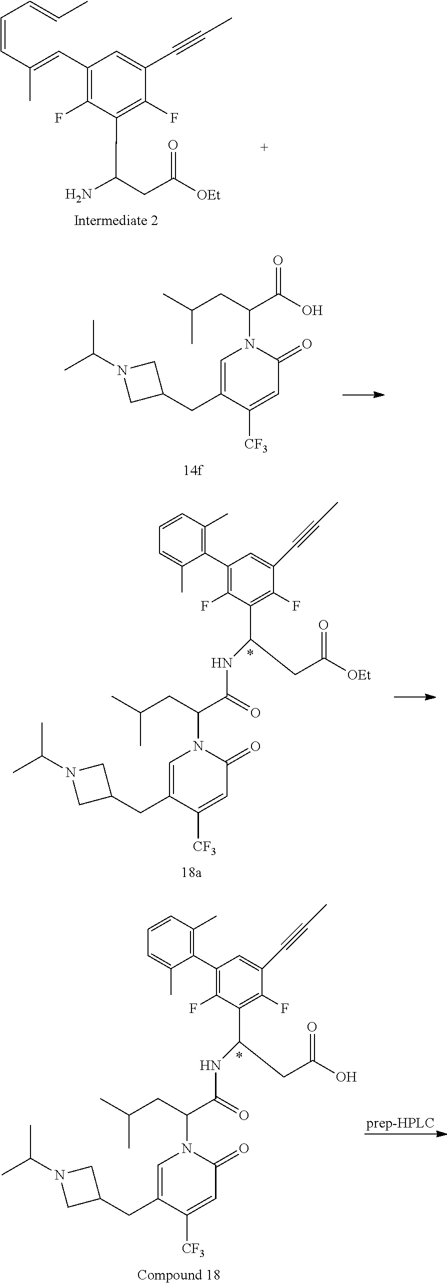

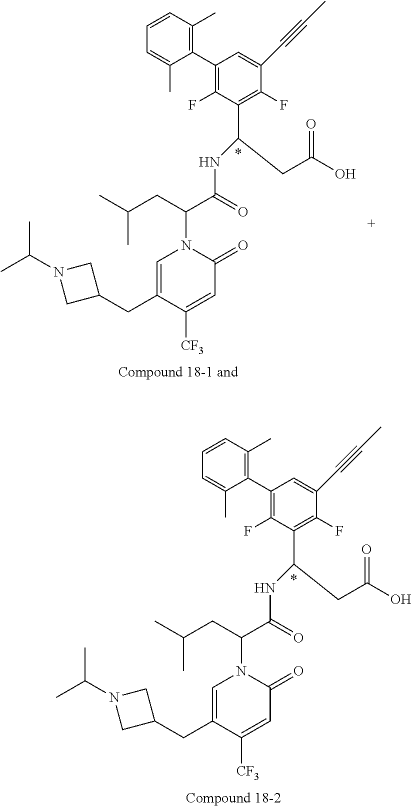 [SMILES: CC#Cc1cc(-c2c(C)cccc2C)c(F)c(C(CC(=O)O)NC(=O)C(CC(C)C)n2cc(CC3CN(C(C)C)C3)c(C(F)(F)F)cc2=O)c1F ; CC#Cc1cc(-c2c(C)cccc2C)c(F)c(C(CC(=O)O)NC(=O)C(CC(C)C)n2cc(CC3CN(C(C)C)C3)c(C(F)(F)F)cc2=O)c1F]

Referring to the preparation method of compound 14, crude compound 18 was obtained, which was separated and purified by prep-HPLC (method 2) to afford the trifluoroacetate salt of compound 18-1 (12 mg, Rt=4.164 min) and the trifluoroacetate salt of compound 18-2 (13 mg, Rt=4.268 min).

### Trifluoroacetate Salt of Compound 18-1:

LC-Ms m/z (ESI): 714.7 [M+H]⁺

¹H NMR (400 MHz, CD₃OD) δ 7.97-7.77 (m, 1H), 7.25-7.18 (m, 1H), 7.18-7.09 (m, 3H), 6.89 (s, 1H), 5.89-5.79 (m, 1H), 5.77-5.65 (m, 1H), 4.34-4.05 (m, 2H), 4.03-3.85 (m, 2H), 3.55-3.36 (m, 1H), 3.24-3.03 (m, 2H), 3.00-2.79 (m, 3H), 2.08 (s, 3H), 2.04 (s, 6H), 1.93-1.71 (m, 2H), 1.39-1.20 (m, 7H), 0.97-0.85 (m, 6H).

### Trifluoroacetate Salt of Compound 18-2:

LC-Ms m/z (ESI): 714.7 [M+H]⁺

¹H NMR (400 MHz, CD₃OD) δ 7.91-7.72 (m, 1H), 7.22-7.15 (m, 1H), 7.14-7.04 (m, 3H), 6.84 (s, 1H), 5.88-5.79 (m, 1H), 5.75-5.65 (m, 1H), 4.33-4.14 (m, 1H), 4.10-3.81 (m, 3H), 3.51-3.35 (m, 1H), 3.22-3.07 (m, 2H), 3.06-2.81 (m, 3H), 2.14-2.04 (m, 4H), 2.02-1.83 (m, 7H), 1.48-1.35 (m, 1H), 1.32-1.17 (m, 6H), 1.04-0.93 (m, 6H).

### Example 19: Preparation of Compound 19

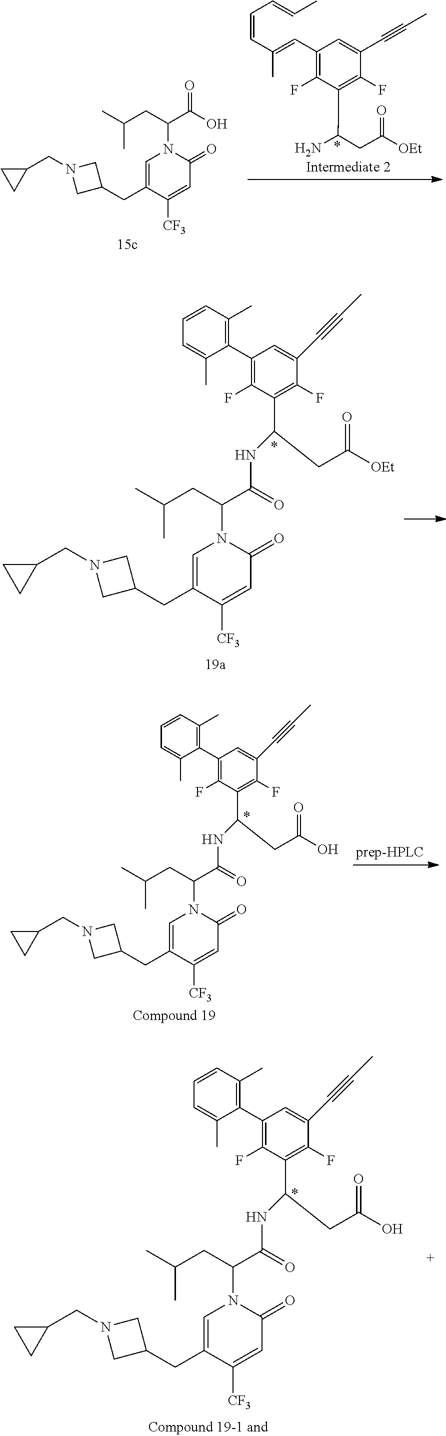 [SMILES: CC(C)CC(C(=O)O)n1cc(CC2CN(CC3CC3)C2)c(C(F)(F)F)cc1=O ; CC#Cc1cc(/C=C(C)/C=C\C=C\C)c(F)c(C(N)CC(=O)OCC)c1F ; CC#Cc1cc(-c2c(C)cccc2C)c(F)c(C(CC(=O)OCC)NC(=O)C(CC(C)C)n2cc(CC3CN(CC4CC4)C3)c(C(F)(F)F)cc2=O)c1F ; CC#Cc1cc(-c2c(C)cccc2C)c(F)c(C(CC(=O)O)NC(=O)C(CC(C)C)n2cc(CC3CN(CC4CC4)C3)c(C(F)(F)F)cc2=O)c1F ; CC#Cc1cc(-c2c(C)cccc2C)c(F)c(C(CC(=O)O)NC(=O)C(CC(C)C)n2cc(CC3CN(CC4CC4)C3)c(C(F)(F)F)cc2=O)c1F]

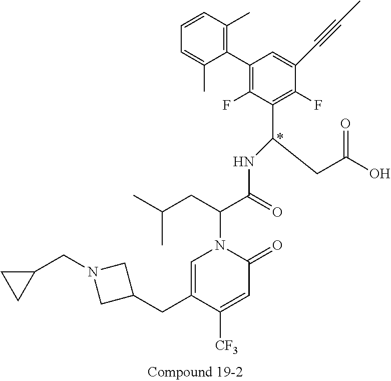 [SMILES: CC#Cc1cc(-c2c(C)cccc2C)c(F)c(C(CC(=O)O)NC(=O)C(CC(C)C)n2cc(CC3CN(CC4CC4)C3)c(C(F)(F)F)cc2=O)c1F]

Referring to the preparation method of compound 14, crude compound 19 was obtained, which was separated and purified by prep-HPLC (method 2) to afford the trifluoroacetate salt of compound 19-1 (18 mg, Rt=4.218 min) and the trifluoroacetate salt of compound 19-2 (19 mg, Rt=4.297 min).

### Trifluoroacetate Salt of Compound 19-1:

LC-Ms m/z (ESI): 726.8 [M+H]⁺

¹H NMR (400 MHz, Methanol-d₄) δ 9.36-9.20 (m, 1H), 7.88-7.69 (m, 1H), 7.23-7.04 (m, 4H), 6.88-6.77 (m, 1H), 5.87-5.76 (m, 1H), 5.75-5.65 (m, 1H), 4.34-3.85 (m, 4H), 3.26-2.87 (m, 7H), 2.12-2.01 (m, 4H), 2.01-1.93 (m, 4H), 1.92-1.84 (m, 3H), 1.46-1.35 (m, 1H), 1.06-1.93 (m, 7H), 0.74-0.64 (m, 2H), 0.47-0.35 (m, 2H).

### Trifluoroacetate Salt of Compound 19-2:

LC-Ms m/z (ESI): 726.8 [M+H]⁺

¹H NMR (400 MHz, Methanol-d₄) δ 9.51-9.33 (m, 1H), 7.94-7.77 (m, 1H), 7.27-7.04 (m, 4H), 6.89 (d, 1H), 5.88-5.78 (m, 1H), 5.76-5.65 (m, 1H), 4.37-3.90 (m, 4H), 3.29-2.82 (m, 7H), 2.08 (s, 3H), 2.04 (s, 6H), 1.92-1.69 (m, 2H), 1.39-1.25 (m, 1H), 1.11-0.96 (m, 1H), 0.96-0.86 (m, 6H), 0.75-0.64 (m, 2H), 0.45-0.36 (m, 2H).

### Example 20: Preparation of Compound 20

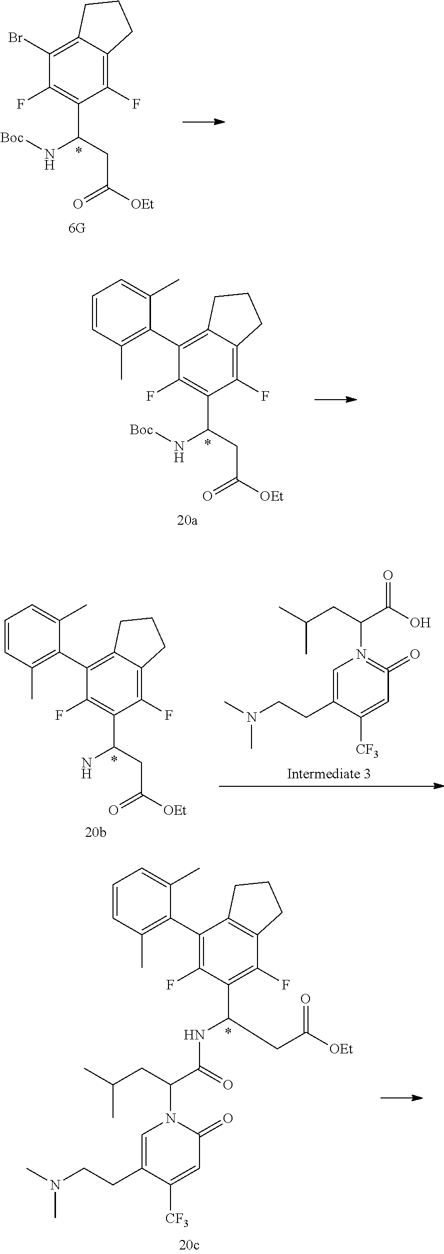

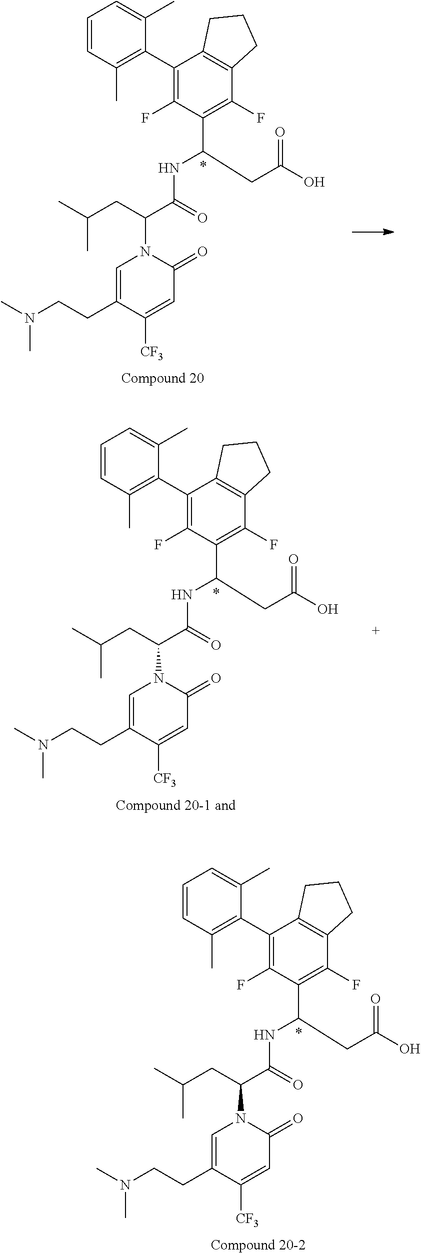 [SMILES: Cc1cccc(C)c1-c1c(F)c(C(CC(=O)O)NC(=O)C(CC(C)C)n2cc(CCN(C)C)c(C(F)(F)F)cc2=O)c(F)c2c1CCC2 ; Cc1cccc(C)c1-c1c(F)c(C(CC(=O)O)NC(=O)[C@@H](CC(C)C)n2cc(CCN(C)C)c(C(F)(F)F)cc2=O)c(F)c2c1CCC2 ; Cc1cccc(C)c1-c1c(F)c(C(CC(=O)O)NC(=O)[C@H](CC(C)C)n2cc(CCN(C)C)c(C(F)(F)F)cc2=O)c(F)c2c1CCC2]

Referring to the preparation method of Example 21, crude compound 20 was obtained, which was separated and purified by prep-HPLC (method 2) to afford the trifluoroacetate salt of compound 20-1 (16 mg, Rt=4.007 min yield: 5.51%) and the trifluoroacetate salt of compound 20-2 (17 mg, Rt=4.095 min, yield: 5.85%).

### Trifluoroacetate Salt of Compound 20-1:

LC-Ms m/z (ESI): 676.7 [M+H]⁺

¹H NMR (400 MHz, Methanol-d₄) δ 9.20 (d, 1H), 7.89 (s, 1H), 7.20-7.04 (m, 3H), 6.82 (s, 1H), 5.82-5.73 (m, 1H), 5.74-5.64 (m, 1H), 3.29-3.23 (m, 2H), 3.16-2.82 (m, 12H), 2.50-2.35 (m, 2H), 2.08-1.88 (m, 7H), 1.86 (s, 3H), 1.47-1.34 (m, 1H), 1.00-0.89 (m, 6H).

### Trifluoroacetate Salt of Compound 20-2:

LC-Ms m/z (ESI): 676.7 [M+H]⁺

¹H NMR (400 MHz, Methanol-d₄) δ 9.33 (d, 1H), 7.91 (s, 1H), 7.20-7.07 (m, 3H), 6.88 (s, 1H), 5.82-5.66 (m, 2H), 3.37-3.30 (m, 2H), 3.08-2.86 (m, 12H), 2.48 (t, 2H), 2.14-2.01 (m, 2H), 1.98 (s, 6H), 1.86-1.73 (m, 2H), 1.36-1.23 (m, 1H), 0.93-0.81 (m, 6H).

### Example 21: Preparation of Compound 21

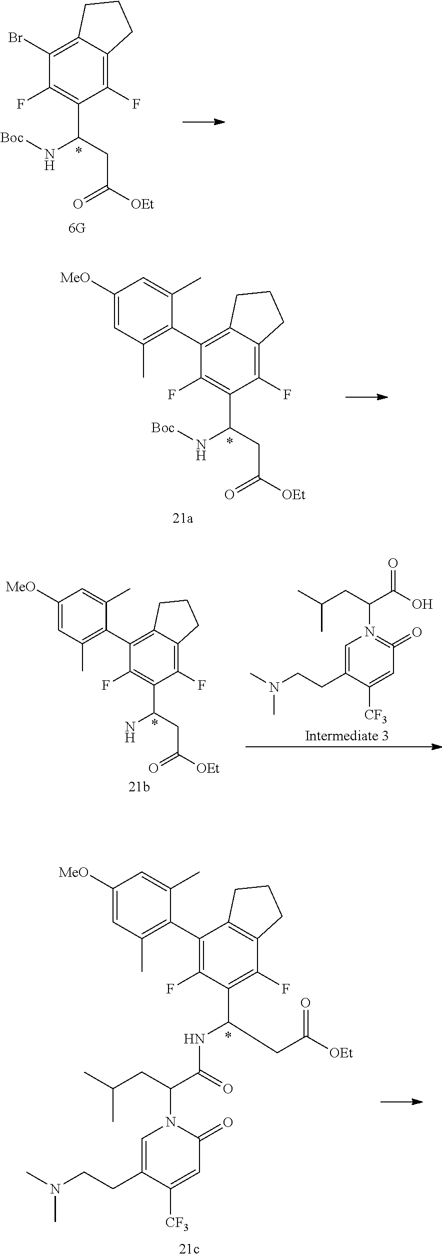

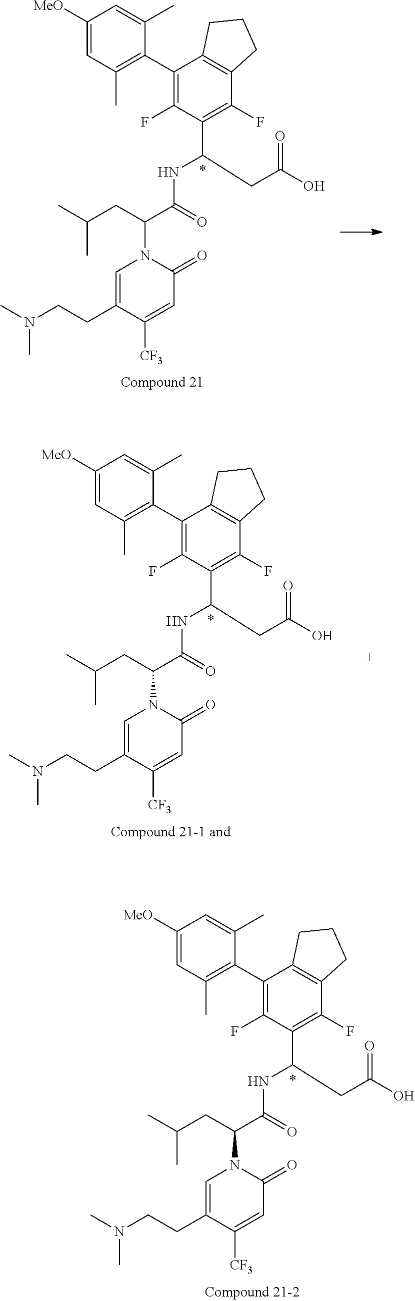 [SMILES: COc1cc(C)c(-c2c(F)c(C(CC(=O)O)NC(=O)C(CC(C)C)n3cc(CCN(C)C)c(C(F)(F)F)cc3=O)c(F)c3c2CCC3)c(C)c1 ; COc1cc(C)c(-c2c(F)c(C(CC(=O)O)NC(=O)[C@@H](CC(C)C)n3cc(CCN(C)C)c(C(F)(F)F)cc3=O)c(F)c3c2CCC3)c(C)c1 ; COc1cc(C)c(-c2c(F)c(C(CC(=O)O)NC(=O)[C@H](CC(C)C)n3cc(CCN(C)C)c(C(F)(F)F)cc3=O)c(F)c3c2CCC3)c(C)c1]

### Step 1: Synthesis of 21a

Under nitrogen protection, 6G (400 mg, 0.89 mmol) was dissolved in 1,4-dioxane (6 mL) and water (0.6 mL). 2.6-Dimethyl-4-methoxy phenylboronic acid (240 mg, 1.33 mmol), XPhos Pd G2 (110 mg, 0.13 mmol) and potassium phosphate (570 mg, 2.67 mmol) were successively added, and the resulting mixture was reacted at 100° C. for 24 h. Extraction was performed by addition of ethyl acetate (80 mL) and the resulting mixture was washed with water (50 mL×2) and saturated sodium chloride aqueous solution (50 mL×1), dried over anhydrous sodium sulfate and filtered and the filtrate was concentrated under reduced pressure and subjected to column chromatography on a silica gel column to afford 21a (230 mg, yield: 51.32%).

LC-Ms m/z (ESI): 404.5 [M+H-Boc]⁺

### Step 2: Synthesis of 21b

21a (230 mg, 0.46 mmol) was dissolved in dichloromethane (6 mL), hydrogen chloride-1,4-dioxane (6 mL) was added, and the reaction was carried out for 1 h. The solvent was removed under reduced pressure to afford the hydrochloride salt of crude 21b.

### Step 3: Synthesis of 21c

Under nitrogen protection, intermediate 3 (160 mg, 0.46 mmol) was dissolved in dry DMF (8 mL), and HATU (350 mg, 0.92 mmol) and DIPEA (230 mg, 1.81 mmol) were successively added. At room temperature, the reaction mixture was stirred for 40 min, and then the hydrochloride salt of 21b (185 mg, 0.46 mmol) was added. The resulting mixture was reacted at room temperature overnight. Ethyl acetate (80 mL) was added and the resulting mixture was successively washed with water (20 mL×2) and saturated sodium chloride aqueous solution (20 mL×1), dried over anhydrous sodium sulfate and filtered and the filtrate was concentrated under reduced pressure and subjected to column chromatography on a silica gel column to afford 21c (240 mg, yield: 71.10%).

LC-Ms m/z (ESI): 734.8[M+H]⁺

### Step 4: Synthesis of Compound 21-1 and Compound 21-2

Compound 21c (240 mg, 0.33 mmol) was dissolved in THF (6 mL) and water (2 mL), lithium hydroxide monohydrate (20 mg, 0.83 mmol) was added, and the resulting mixture was reacted at room temperature for 30 min. PH was adjusted to 5-6 with 1 N hydrochloric acid, and the resulting solution was concentrated under reduced pressure to afford crude compound 21, which was separated and purified by prep-HPLC (method 2) to afford the trifluoroacetate salt of compound 21-1 (19 mg, Rt=3.973 min, yield: 8.16%) and the trifluoroacetate salt of compound 21-2 (20 mg, Rt=4.051 min, yield: 8.59%).

### Trifluoroacetate Salt of Compound 21-1:

LC-Ms m/z (ESI): 706.7 [M+H]⁺

¹H NMR (400 MHz, Methanol-d₄) δ 7.89 (s, 1H), 6.82 (s, 1H), 6.67 (s, 2H), 5.82-5.74 (m, 1H), 5.72-5.63 (m, 1H), 3.78 (s, 3H), 3.32-3.24 (m, 2H), 3.14-2.83 (m, 12H), 2.49-2.38 (m, 2H), 2.08-1.92 (m, 4H), 1.90 (s, 3H), 1.83 (s, 3H), 1.47-1.35 (m, 1H), 1.01-0.86 (m, 6H).

### Trifluoroacetate Salt of Compound 21-2:

LC-Ms m/z (ESI): 706.7 [M+H]⁺

¹H NMR (400 MHz, Methanol-d₄) δ 9.33 (d, 1H), 7.91 (s, 1H), 6.88 (s, 1H), 6.70 (s, 2H), 5.82-5.75 (m, 1H), 5.74-5.63 (m, 1H), 3.79 (s, 3H), 3.36-3.29 (m, 2H), 3.10-2.83 (m, 12H), 2.53-2.43 (m, 2H), 2.13-2.01 (m, 2H), 1.95 (s, 6H), 1.87-1.74 (m, 2H), 1.35-1.23 (m, 1H), 0.93-0.82 (m, 6H)

Compound 21-A has the structure of

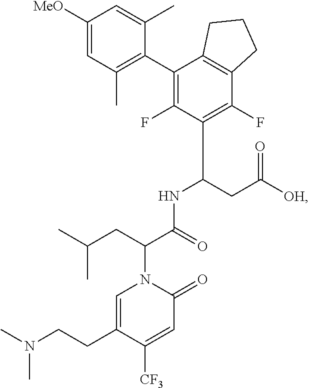 [SMILES: COc1cc(C)c(-c2c(F)c(C(CC(=O)O)NC(=O)C(CC(C)C)n3cc(CCN(C)C)c(C(F)(F)F)cc3=O)c(F)c3c2CCC3)c(C)c1]

which is the racemate of 4 chiral isomers mixed in equal proportions.

### Example 22: Preparation of Compound 22

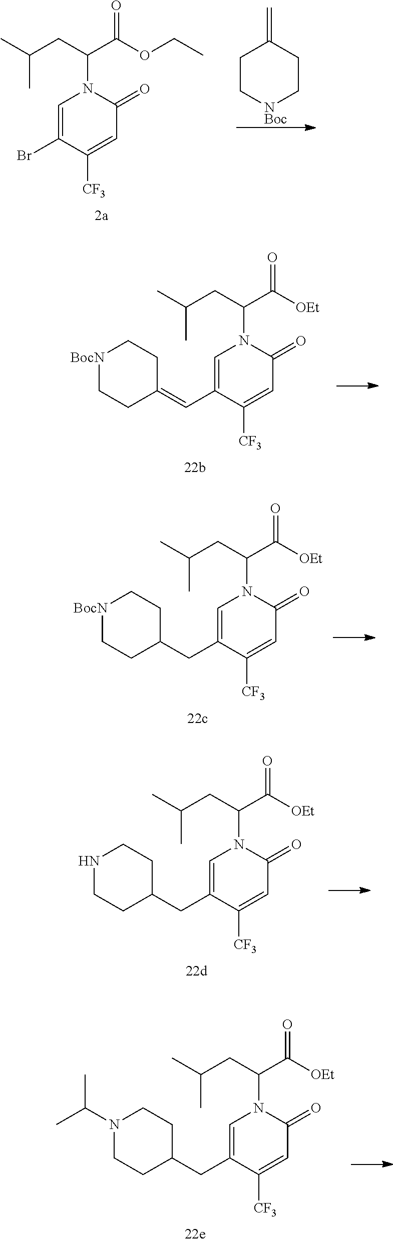

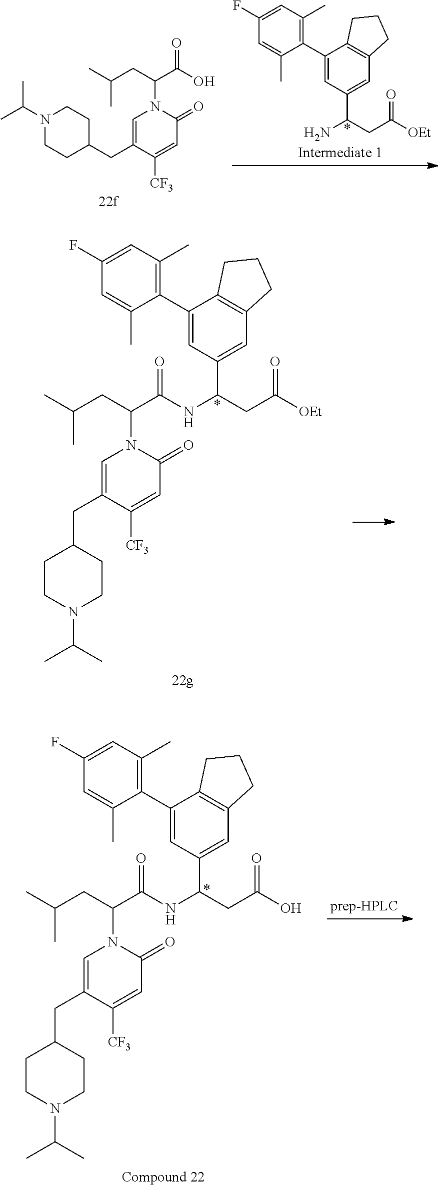 [SMILES: CC(C)CC(C(=O)O)n1cc(CC2CCN(C(C)C)CC2)c(C(F)(F)F)cc1=O ; CCOC(=O)CC(N)c1cc2c(c(-c3c(C)cc(F)cc3C)c1)CCC2 ; CCOC(=O)CC(NC(=O)C(CC(C)C)n1cc(CC2CCN(C(C)C)CC2)c(C(F)(F)F)cc1=O)c1cc2c(c(-c3c(C)cc(F)cc3C)c1)CCC2 ; Cc1cc(F)cc(C)c1-c1cc(C(CC(=O)O)NC(=O)C(CC(C)C)n2cc(CC3CCN(C(C)C)CC3)c(C(F)(F)F)cc2=O)cc2c1CCC2]

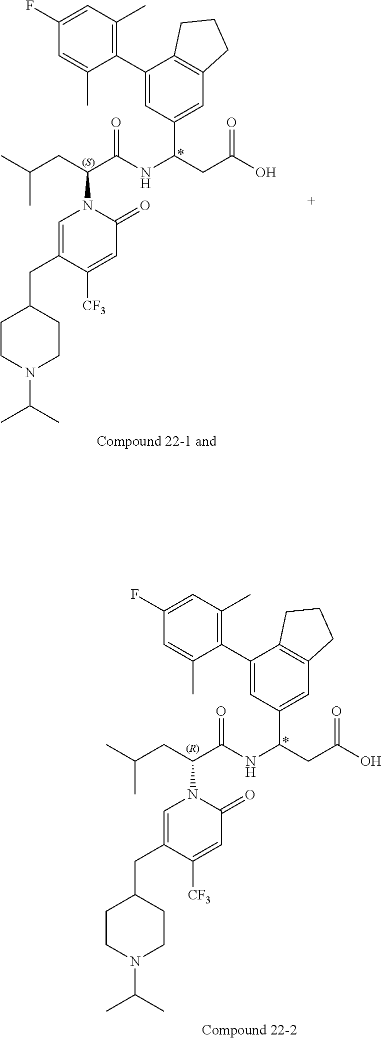

Referring to the preparation method of compound 14, crude compound 22 was obtained, which was separated and purified by prep-HPLC (method 2) to afford the trifluoroacetate salt of compound 22-1 (10 mg, Rt=4.334 min) and the trifluoroacetate salt of compound 22-2 (10 mg, Rt 4.411 min).

### Trifluoroacetate Salt of Compound 22-1:

LC-Ms m/z (ESI): 726.4 [M+H]⁺

¹H NMR (400 MHz, Methanol-d₄) δ 7.74 (s, 1H), 7.18 (s, 1H), 6.86-6.70 (m, 4H), 5.76 (t, 1H), 5.33 (t, 1H), 3.50-3.35 (m, 3H), 3.06-2.97 (m, 1H), 2.96-2.81 (m, 5H), 2.61-2.49 (m, 2H), 2.40 (t, 2H), 2.07-1.90 (m, 6H), 1.86 (s, 3H), 1.81 (s, 3H), 1.55-1.37 (m, 4H), 1.34 (s, 3H), 1.32 (s, 3H), 1.00-0.92 (m, 6H).

### Trifluoroacetate Salt of Compound 22-2:

LC-Ms m/z (ESI): 726.4 [M+H]⁺

¹H NMR (400 MHz, Methanol-d₄) δ 9.12 (d, 1H), 7.79 (s, 1H), 7.23 (d, 1H), 6.88-6.79 (m, 4H), 5.76 (t, 1H), 5.31 (q, 1H), 3.52-3.41 (m, 3H), 3.10-2.94 (m, 4H), 2.86-2.78 (m, 2H), 2.65-2.42 (m, 4H), 2.11-1.98 (m, 4H), 1.96-1.90 (m, 6H), 1.83-1.77 (m, 2H), 1.57-1.26 (m, 10H), 0.92-0.83 (m, 6H).

### Example 23: Preparation of Compound 23

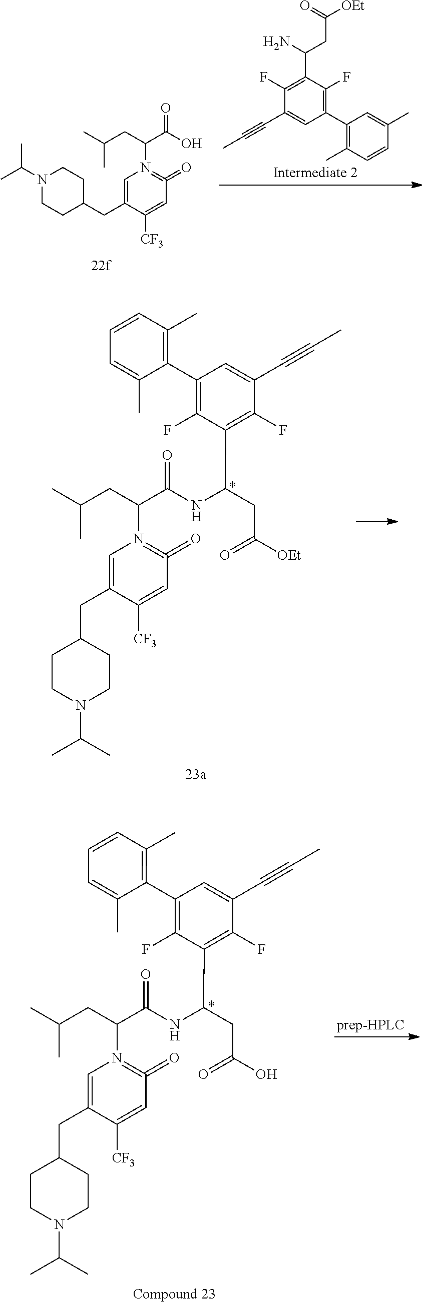

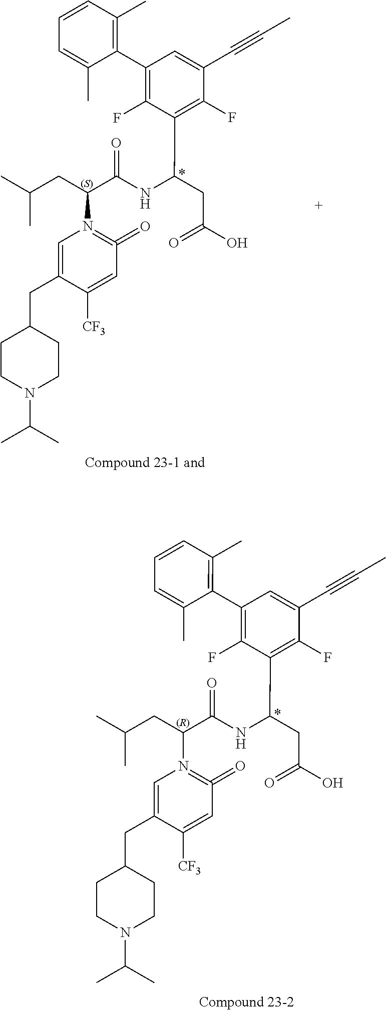

Referring to the preparation method of compound 14, crude compound 23 was obtained, which was separated and purified by prep-HPLC (method 2) to afford the trifluoroacetate salt of compound 23-1 (10 mg, Rt=4.233 min) and the trifluoroacetate salt of compound 23-2 (10 mg, Rt=4.411 min).

### Trifluoroacetate Salt of Compound 23-1:

LC-Ms m/z (ESI): 742.4 [M+H]⁺

¹H NMR (400 MHz, Methanol-d₄) δ 9.29 (d, 1H), 7.74 (s, 1H), 7.20-7.03 (m, 4H), 6.81 (s, 1H), 5.84-5.75 (m, 1H), 5.73-5.63 (m, 1H), 3.51-3.38 (m, 2H), 3.13-2.87 (m, 5H), 2.60-2.48 (m, 2H), 2.04 (s, 3H), 2.02-1.89 (m, 6H), 1.88-1.71 (m, 5H), 1.55-1.25 (m, 10H), 1.00-0.89 (m, 6H).

### Trifluoroacetate Salt of Compound 23-2:

LC-Ms m/z (ESI): 742.4 [M+H]⁺

¹H NMR (400 MHz, Methanol-d₄) δ 7.79 (s, 1H), 7.24-7.07 (m, 4H), 6.86 (s, 1H), 5.83-5.74 (m, 1H), 5.76-5.64 (m, 1H), 3.55-3.40 (m, 3H), 3.12-2.87 (m, 4H), 2.65-2.47 (m, 2H), 2.15-1.96 (m, 11H), 1.90-1.72 (m, 3H), 1.58-1.44 (m, 2H), 1.41-1.21 (m, 7H), 0.95-0.81 (m, 6H).

### Example 24: Preparation of Compound 24

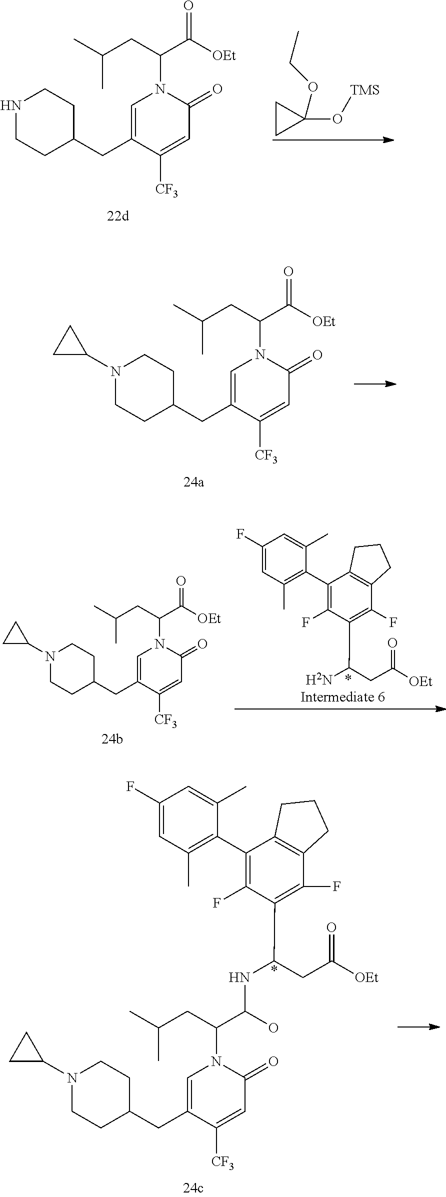 [SMILES: CCOC(=O)C(CC(C)C)n1cc(CC2CCNCC2)c(C(F)(F)F)cc1=O ; CCOC1(O[Si](C)(C)C)CC1 ; CCOC(=O)C(CC(C)C)n1cc(CC2CCN(C3CC3)CC2)c(C(F)(F)F)cc1=O ; CCOC(=O)C(CC(C)C)n1cc(CC2CCN(C3CC3)CC2)c(C(F)(F)F)cc1=O ; CCOC(=O)CC([2NH2])c1c(F)c2c(c(-c3c(C)cc(F)cc3C)c1F)CCC2 ; CCOC(=O)CC(NC(O)C(CC(C)C)n1cc(CC2CCN(C3CC3)CC2)c(C(F)(F)F)cc1=O)c1c(F)c2c(c(-c3c(C)cc(F)cc3C)c1F)CCC2]

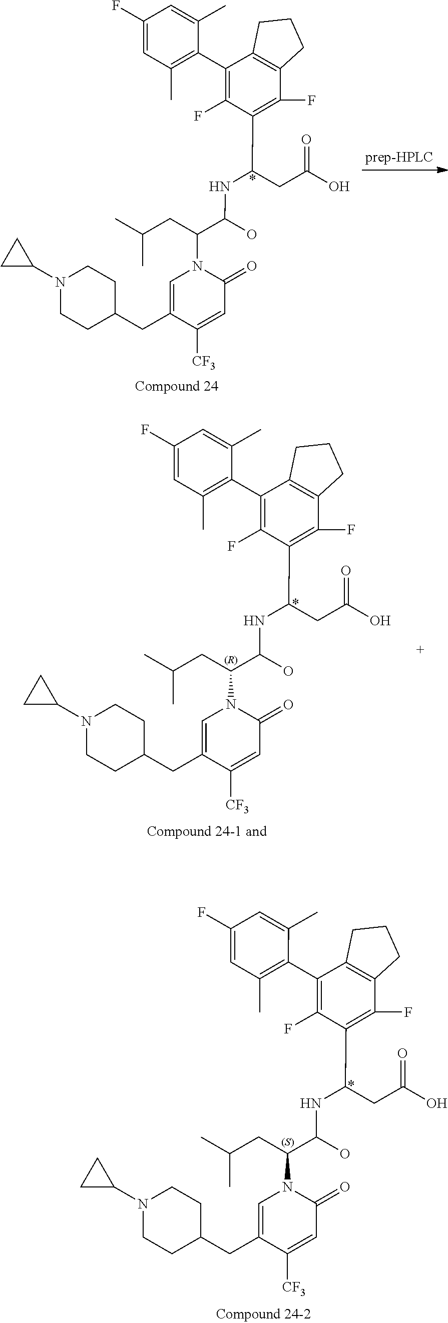

Referring to the preparation method of compound 14, crude compound 24 was obtained, which was separated and purified by prep-HPLC (method 1) to afford compound 24-1 (34 mg, Rt=4.300 min) and compound 24-2 (35 mg, Rt=4.374 min).

### Compound 24-1:

LC-Ms m/z (ESI): 760.3 [M+H]⁺

¹H NMR (400 MHz, Methanol-d₄) δ 7.71 (s, 1H), 6.89-6.82 (m 2H), 6.81 (s, 1H), 5.81-5.74 (m, 1H), 5.73-5.67 (m, 1H), 3.31-3.24 (m, 2H), 3.04-2.93 (m, 3H), 2.80-2.73 (m, 1H), 2.70-2.53 (m, 2H), 2.54-2.43 (m, 4H), 2.23-2.16 (m, 1H), 2.12-2.02 (m, 2H), 2.01-1.91 (m, 5H), 1.86 (s, 3H), 1.82-1.77 (m, 2H), 1.70-1.61 (m, 1H), 1.41-1.25 (m, 3H), 0.98-0.93 (m, 6H), 0.77-0.65 (m, 4H).

### Compound 24-2:

LC-Ms m/z (ESI): 760.3 [M+H]⁺

¹H NMR (400 MHz, Methanol-d₄) δ 7.78 (s, 1H), 6.91 (s, 1H), 6.89-6.85 (m, 2H), 5.81-5.75 (m, 1H), 5.75-5.68 (m, 1H), 3.47-3.38 (m, 2H), 3.01 (t, 2H), 2.94-2.84 (m, 1H), 2.82-2.58 (m, 4H), 2.57-2.47 (m, 3H), 2.33-2.26 (m, 1H), 2.15-2.05 (m, 2H), 2.02-1.96 (m, 6H), 1.89-1.71 (m, 4H), 1.59-1.50 (m, 1H), 1.40-1.24 (m, 3H), 0.91-0.85 (m, 6H), 0.84-0.79 (m, 2H), 0.77-0.71 (m, 2H).

### Example 25: Preparation of Compound 25

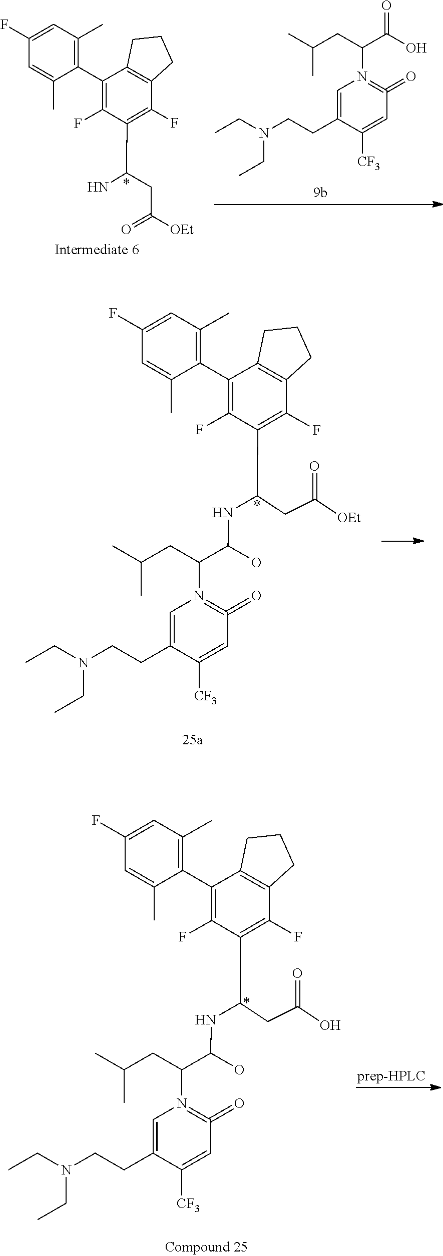

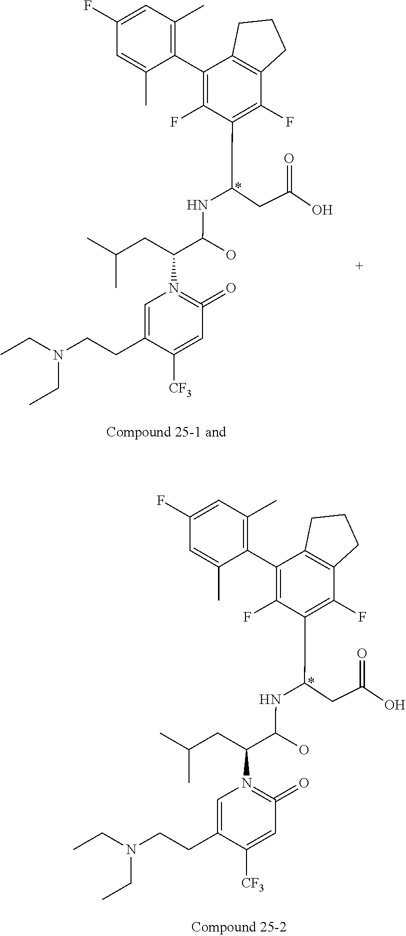

### Step 1: Synthesis of 25a

Under nitrogen protection, 9b (140 mg 0.33 mmol) and intermediate 6 (124 mg, 0.33 mmol) were dissolved in dry DMF (3.0 mL). EDCI (126 mg, 0.66 mmol), HOBT (90 mg, 0.66 mmol) and DIPEA (128 mg, 0.99 mmol) were added, and the resulting mixture was reacted at room temperature overnight. Ethyl acetate (80 mL) was added, and the resulting mixture was successively washed with water (20 mL×2) and saturated sodium chloride aqueous solution (20 mL×1), dried over anhydrous sodium sulfate and filtered, and the filtrate was concentrated under reduced pressure and then subjected to column chromatography on a silica gel column to afford crude 25a (250 mg).

LC-Ms m/z (ESI): 750.2 [M+H]⁺

### Step 2: Preparation of Compounds 25-1 and 25-2

The above-mentioned crude 25a (250 mg) was dissolved in THF (4 mL) and water (1 mL), lithium hydroxide monohydrate (45 mg, 1.08 mmol) was added, and the resulting mixture was reacted at room temperature for 5 h. PH was adjusted to 5-6 with 1 N hydrochloric acid, and the resulting solution was concentrated under reduced pressure. The residue was separated and purified by prep-HPLC (method 1) to afford compound 25-1 (29 mg, Rt=4.237 min, two-step yield: 10.86%) and compound 25-2 (36 mg, Rt=4.294 min, two-step yield: 13.48%).

### Compound 25-1:

LC-Ms m/z (ESI): 722.3 [M+H]⁺

¹H NMR (400 MHz, Methanol-d₄) δ 7.91 (s, 1H), 6.90-6.76 (m, 3H), 5.78-5.68 (m, 2H), 3.26-3.11 (m, 6H), 3.02-2.89 (m, 5H), 2.71-2.62 (m, 1H), 2.50-2.43 (m, 2H), 2.11-1.98 (m, 4H), 1.97 (s, 3H), 1.90 (s, 3H), 1.45-1.34 (m, 1H), 1.34-1.28 (m, 6H), 0.99-0.90 (m, 6H).

### Compound 25-2:

LC-Ms m/z (ESI): 722.3 [M+H]⁺

¹H NMR (400 MHz, Methanol-d₄) δ 7.88 (s, 1H), 6.93-6.86 (m, 3H), 5.83-5.75 (m, 1H), 5.70-5.61 (m, 1H), 3.29-3.15 (m, 6H), 3.05-2.94 (m, 4H), 2.89-2.79 (m, 1H), 2.64-2.55 (m, 1H), 2.56-2.46 (m, 2H), 2.14-2.04 (m, 2H), 2.02-1.97 (m, 6H), 1.95-1.87 (m, 1H), 1.78-1.69 (m, 1H), 1.39-1.28 (m, 7H), 0.93-0.86 (m, 6H).

### Example 26: Preparation of Compound 26

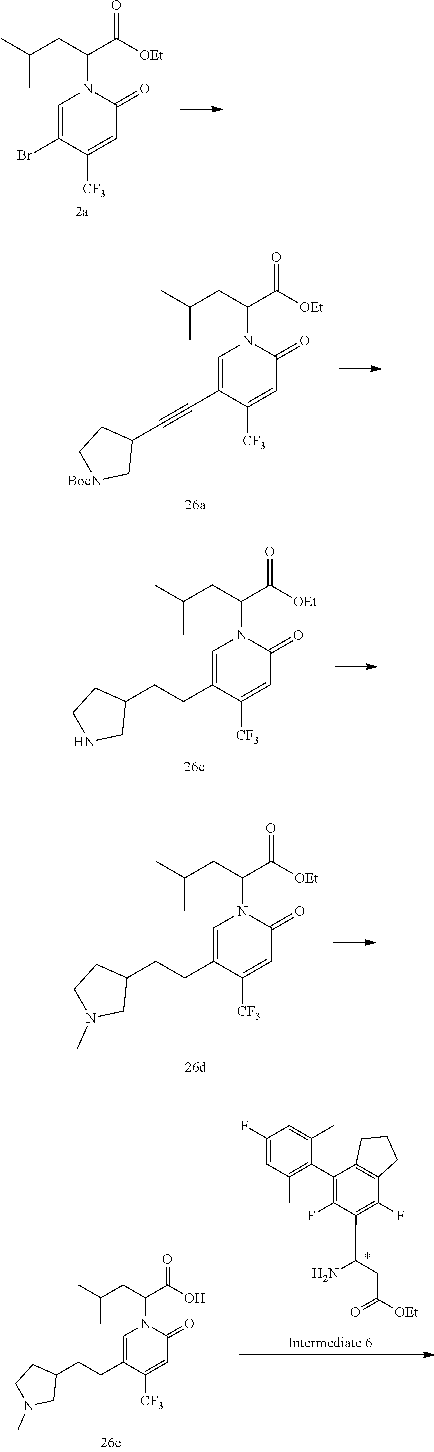 [SMILES: CCOC(=O)C(CC(C)C)n1cc(Br)c(C(F)(F)F)cc1=O ; CCOC(=O)C(CC(C)C)n1cc(C#CC2CCN(C(=O)OC(C)(C)C)C2)c(C(F)(F)F)cc1=O ; CCOC(=O)C(CC(C)C)n1cc(CCC2CCNC2)c(C(F)(F)F)cc1=O ; CCOC(=O)C(CC(C)C)n1cc(CCC2CCN(C)C2)c(C(F)(F)F)cc1=O ; CC(C)CC(C(=O)O)n1cc(CCC2CCN(C)C2)c(C(F)(F)F)cc1=O ; CCOC(=O)CC(N)c1c(F)c2c(c(-c3c(C)cc(F)cc3C)c1F)CCC2]

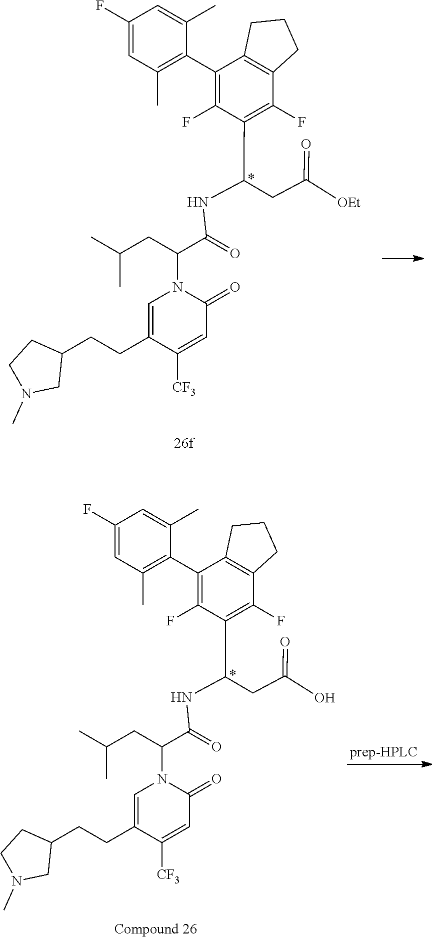 [SMILES: CCOC(=O)CC(NC(=O)C(CC(C)C)n1cc(CCC2CCN(C)C2)c(C(F)(F)F)cc1=O)c1c(F)c2c(c(-c3c(C)cc(F)cc3C)c1F)CCC2 ; Cc1cc(F)cc(C)c1-c1c(F)c(C(CC(=O)O)NC(=O)C(CC(C)C)n2cc(CCC3CCN(C)C3)c(C(F)(F)F)cc2=O)c(F)c2c1CCC2]

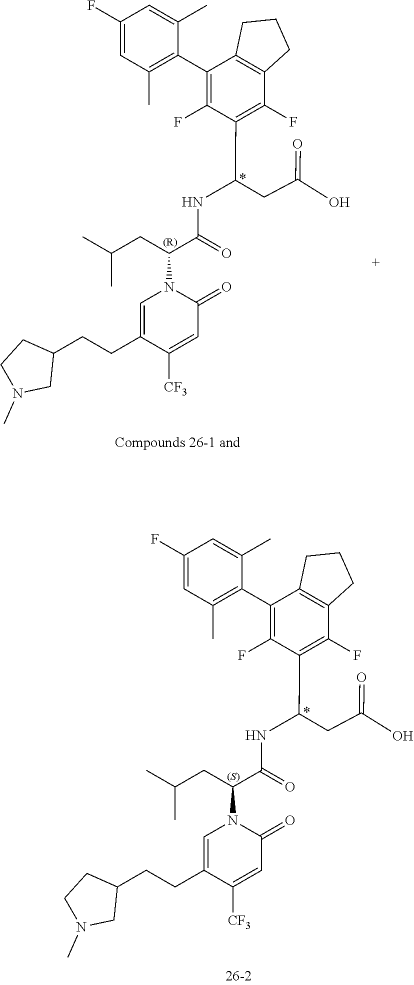

### Step 1: Synthesis of 26a

Under nitrogen protection, 2a (1.68 g, 4.72 mmol) was dissolved in DMF (29 mL) and water (2.9 mL). 1-BOC-3-ethynyl pyrrolidine (1.84 g, 9.44 mmol), Copper iodide (3.6 g, 18.88 mmol), lithium chloride (1.6 g, 37.76 mmol) and PdCl2(dppf) (578 mg, 0.71 mmol) were successively added, and the resulting mixture was reacted at 80° C. for 5 h. The reaction system was filtered through celite, ethyl acetate (100 mL) was added and the resulting mixture was successively washed with water (30 mL×2) and saturated sodium chloride aqueous solution (30 mL×1), dried over anhydrous sodium sulfate and filtered and the filtrate was concentrated under reduced pressure and subjected to column chromatography on a silica gel column to afford 26a (1.7 g, yield: 72.24%).

LC-Ms m/z (ESI): 443.2 [M+H-t-Bu]⁺

### Step 2: Synthesis of 26b

Under nitrogen protection, 26a (1.3 g, 2.61 mmol) was dissolved in methanol (22 mL), and 10% w/w Pd/C (2.2 g, 2.09 mmol) was added. The mixture was subjected to hydrogen replacement three times, and reacted at room temperature under the protection of hydrogen balloon for 0.5 h. The solid was removed by filtration and the filtrate was concentrated to afford crude 26b (1.3 g).

LC-Ms m/z (ESI): 447.2 [M+H-t-Bu]⁺

### Step 3: Synthesis of 26c

The crude 26b (1.3 g) from the previous step was dissolved in dichloromethane (14 mL), hydrogen chloride-1,4-dioxane (10 mL) was added, and the reaction was carried out for 3 h. The solvent was removed under reduced pressure to afford crude 26c (1.4 g).

LC-Ms m/z (ESI): 403.6 [M+H]⁺

### Step 4: Synthesis of 26d

The crude 26c (1.4 g, 3.48 mmol) was dissolved in DCE (23 mL), paraformaldehyde (209 mg, 6.96 mmol), acetic acid (105 mg, 1.74 mmol) and sodium triacetoxyborohydride (1475 mg, 6.96 mmol) were successively added, and the resulting mixture was reacted at 60° C. for 16 h. Dichloromethane (80 mL) and water (30 mL) were added, and the resulting mixture was washed with saturated sodium chloride aqueous solution (30 mL×1), dried over anhydrous sodium sulfate and filtered and the filtrate was concentrated under reduced pressure and subjected to column chromatography on a silica gel column to afford 26d (750 mg, three-step yield: 51.75%).

LC-Ms m/z (ESI): 417.6 [M+H]⁺

### Step 5: Synthesis of 26e

26d (780 mg, 1.87 mmol) was dissolved in THF (8 mL) and water (2 mL), lithium hydroxide monohydrate (235 mg, 5.61 mmol) was added, and the resulting mixture was reacted at room temperature for 3 h. The reaction mixture was adjusted with 1 N hydrochloric acid to pH 5-6 and concentrated under reduced pressure to afford crude compound, which was separated by C18 column (mobile phase A: acetonitrile, and mobile phase B: 0.5% aqueous NH₄HCO₃, gradient elution from 10% to 40% of A in B) to afford 26e (620 mg, 85.36%).

LC-Ms m/z (ESI): 389.4 [M+H]⁺

### Step 6: Synthesis of 26f

Under nitrogen protection, 26e (155 mg, 0.40 mmol) and intermediate 6 (157 mg, 0.40 mmol) were dissolved in dry DMF (3.0 mL). EDCI (153 mg, 0.80 mmol), HOBT (108 mg, 0.80 mmol) and DIPEA (155 mg, 1.20 mmol) were added, and the resulting mixture was reacted at room temperature overnight. Ethyl acetate (80 mL) was added, and the resulting mixture was successively washed with water (20 mL×2) and saturated sodium chloride aqueous solution (20 mL×1), dried over anhydrous sodium sulfate and filtered, and the filtrate was concentrated under reduced pressure and then subjected to column chromatography on a silica gel column to afford 26f (189 mg, 62.02%).

LC-Ms m/z (ESI): 762.7 [M+H]⁺

### Step 7: Preparation of Compounds 26-1 and 26-2

26f (189 mg, 0.25 mmol) was dissolved in THF (4 mL) and water (1 mL), lithium hydroxide monohydrate (32 mg, 0.75 mmol) was added, and the resulting mixture was reacted at room temperature for 5 h. PH was adjusted to 5-6 with 1 N hydrochloric acid, and the resulting solution was concentrated under reduced pressure to afford crude compound 26, which was separated and purified by prep-HPLC (method 2) to afford the trifluoroacetate salt of compound 26-1 (30 mg, Rt=4.218 min, two-step yield: 16.35%) and the trifluoroacetate salt of compound 26-2 (30 mg, Rt=4.305 min, two-step yield: 16.35%).

### Trifluoroacetate Salt of Compound 26-1:

LC-Ms m/z (ESI): 734.3 [M+H]⁺

¹H NMR (400 MHz, Methanol-d₄) δ 9.16 (t, 1H), 7.76 (d, 1H), 6.91-6.83 (m, 2H), 6.81 (s, 1H), 5.84-5.76 (m, 1H), 5.75-5.67 (m, 1H), 3.87-3.63 (m, 2H), 3.29-3.01 (m, 3H), 3.01-2.86 (m, 5H), 2.81-2.53 (m, 3H), 2.53-2.42 (m, 2H), 2.38-2.18 (m, 2H), 2.12-2.03 (m, 3H), 2.02-1.99 (m, 1H), 1.94 (d, 3H), 1.89 (d, 3H), 1.82-1.69 (m, 2H), 1.45-1.33 (m, 2H), 1.01-0.93 (m, 6H).

### Trifluoroacetate Salt of Compound 26-2:

LC-Ms m/z (ESI): 734.3 [M+H]⁺

¹H NMR (400 MHz, Methanol-d₄) δ 9.32 (d, 1H), 7.85 (s, 1H), 6.96-6.84 (m, 3H), 5.80 (t, 1H), 5.75-5.66 (m, 1H), 3.88-3.64 (m, 2H), 3.25-2.83 (m, 8H), 2.82-2.56 (m, 3H), 2.54-2.45 (m, 2H), 2.41-2.18 (m, 1H), 2.17-2.06 (m, 2H), 2.00 (s, 6H), 1.90-1.70 (m, 5H), 1.40-1.23 (m, 2H), 0.95-0.83 (m, 6H).

### Example 27: Preparation of Compound 27

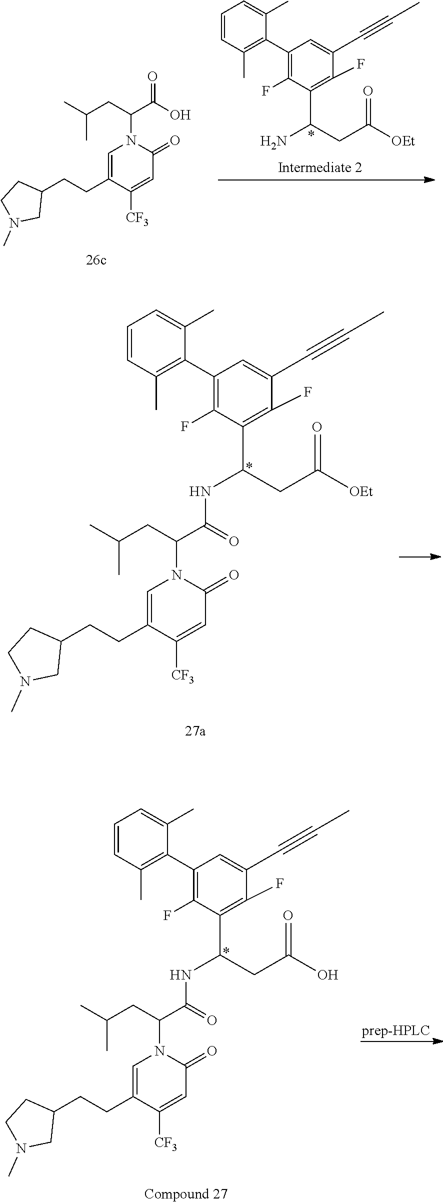 [SMILES: CC(C)CC(C(=O)O)n1cc(CCC2CCN(C)C2)c(C(F)(F)F)cc1=O ; CC#Cc1cc(-c2c(C)cccc2C)c(F)c(C(N)CC(=O)OCC)c1F ; CC#Cc1cc(-c2c(C)cccc2C)c(F)c(C(CC(=O)OCC)NC(=O)C(CC(C)C)n2cc(CCC3CCN(C)C3)c(C(F)(F)F)cc2=O)c1F ; CC#Cc1cc(-c2c(C)cccc2C)c(F)c(C(CC(=O)O)NC(=O)C(CC(C)C)n2cc(CCC3CCN(C)C3)c(C(F)(F)F)cc2=O)c1F]

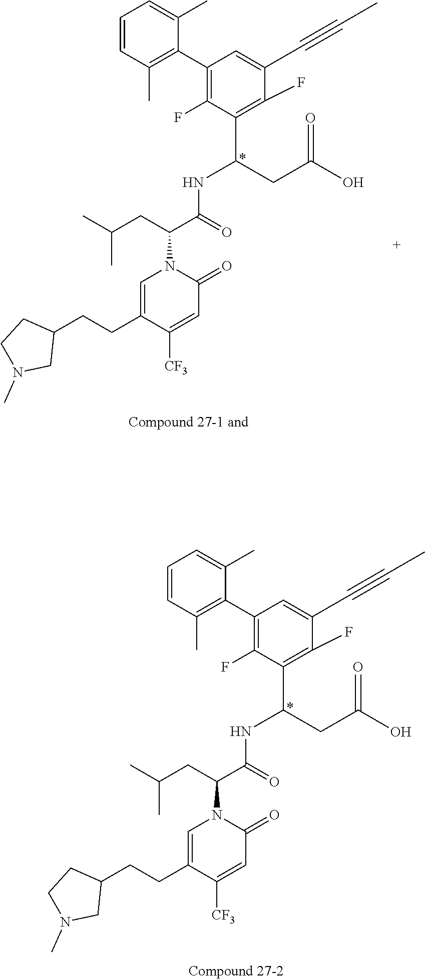 [SMILES: CC#Cc1cc(-c2c(C)cccc2C)c(F)c(C(CC(=O)O)NC(=O)[C@@H](CC(C)C)n2cc(CCC3CCN(C)C3)c(C(F)(F)F)cc2=O)c1F ; CC#Cc1cc(-c2c(C)cccc2C)c(F)c(C(CC(=O)O)NC(=O)[C@H](CC(C)C)n2cc(CCC3CCN(C)C3)c(C(F)(F)F)cc2=O)c1F]

Referring to the preparation method of compound 26, crude compound 27 was obtained, which was separated and purified by prep-HPLC (method 2) to afford the trifluoroacetate salt of compound 27-1 (58 mg, Rt=4.134 min) and the trifluoroacetate salt of compound 27-2 (86 mg, Rt=4.243 min).

### Trifluoroacetate Salt of Compound 27-1:

LC-Ms m/z (ESI): 714.3 [M+H]⁺

¹H NMR (400 MHz, Methanol-d₄) δ 9.27 (t, 1H), 7.75 (s, 1H), 7.19 (t, 1H), 7.13-7.04 (m, 3H), 6.82 (s, 1H), 5.83-5.74 (m, 1H), 5.74-5.63 (m, 1H), 3.87-3.55 (m, 2H), 3.26-3.02 (m, 2H), 3.00-2.88 (m, 4H), 2.81-2.46 (m, 3H), 2.46-2.15 (m, 2H), 2.06 (s, 3H), 2.00-1.91 (m, 4H), 1.90-1.84 (m, 4H), 1.79-1.66 (m, 2H), 1.47-1.33 (m, 2H), 1.01-0.92 (m, 6H).

### Trifluoroacetate Salt of Compound 27-2:

LC-Ms m/z (ESI): 714.3 [M+H]⁺

¹H NMR (400 MHz, Methanol-d₄) δ 9.47-9.37 (m, 1H), 7.84 (d, 1H), 7.26-7.09 (m, 4H), 6.87 (s, 1H), 5.86-5.76 (m, 1H), 5.76-5.65 (m, 1H), 3.90-3.54 (m, 2H), 3.25-3.03 (m, 2H), 3.02-2.88 (m, 4H), 2.84-2.51 (m, 3H), 2.52-2.18 (m, 2H), 2.14-2.00 (m, 9H), 1.92-1.60 (m, 5H), 1.42-1.22 (m, 2H), 0.96-0.82 (m, 6H).

### Example 28: Preparation of Compound 28

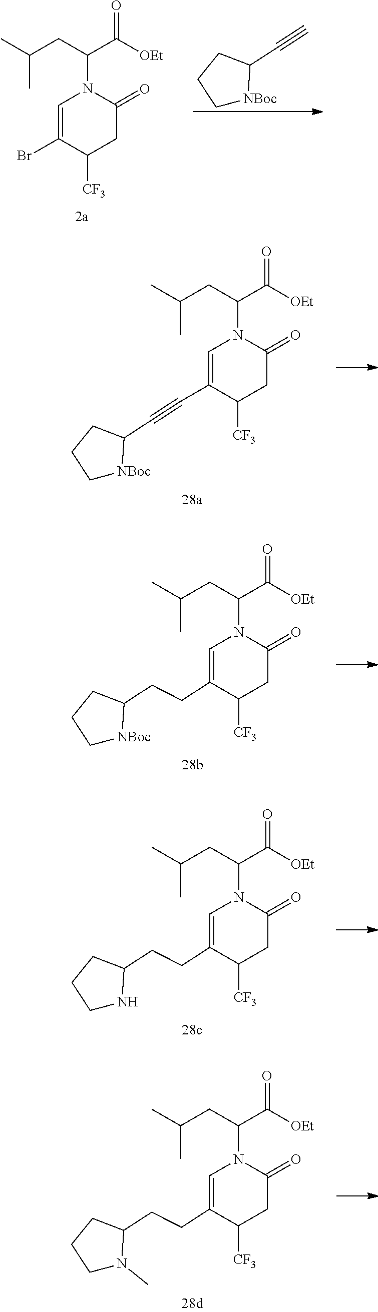 [SMILES: CCOC(=O)C(CC(C)C)N1C=C(Br)C(C(F)(F)F)CC1=O ; C#CC1CCCN1C(=O)OC(C)(C)C ; CCOC(=O)C(CC(C)C)N1C=C(C#CC2CCCN2C(=O)OC(C)(C)C)C(C(F)(F)F)CC1=O ; CCOC(=O)C(CC(C)C)N1C=C(CCC2CCCN2C(=O)OC(C)(C)C)C(C(F)(F)F)CC1=O ; CCOC(=O)C(CC(C)C)N1C=C(CCC2CCCN2)C(C(F)(F)F)CC1=O ; CCOC(=O)C(CC(C)C)N1C=C(CCC2CCCN2C)C(C(F)(F)F)CC1=O]

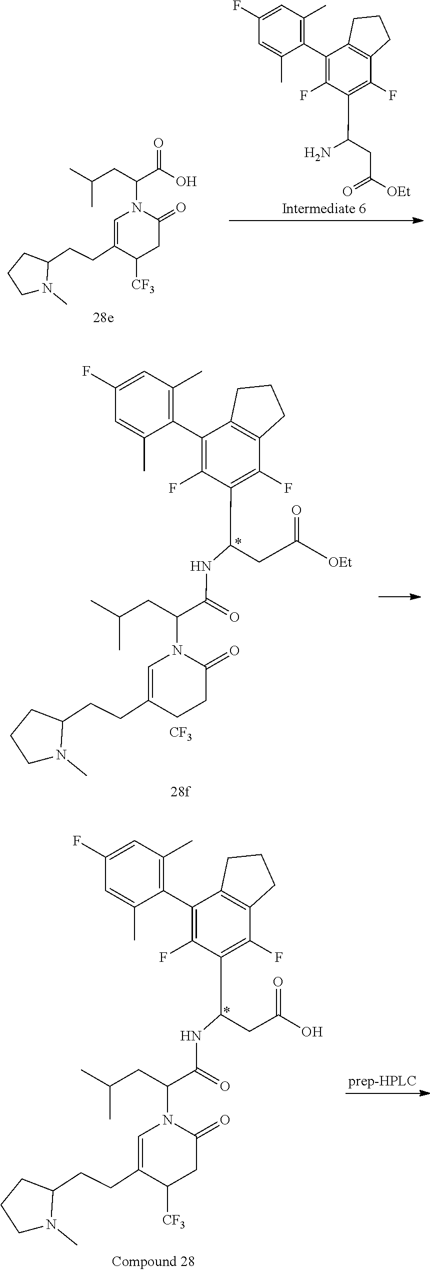

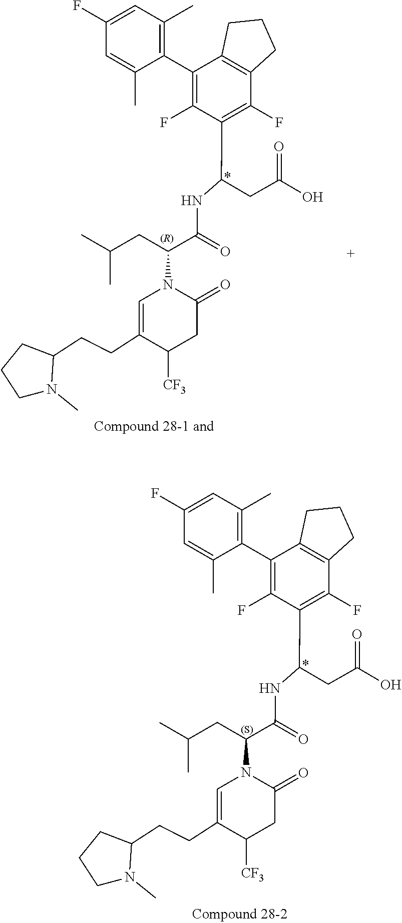

Referring to the preparation method of compound 26, crude compound 28 was obtained, which was separated and purified by prep-HPLC (method 2) to afford the trifluoroacetate salt of compound 28-1 (37 mg, Rt=4.218 min) and the trifluoroacetate salt of compound 28-2 (50 mg, Rt=4.343 min).

### Trifluoroacetate Salt of Compound 28-1:

LC-Ms m/z (ESI): 734.2 [M+H]⁺

¹H NMR (400 MHz, Methanol-d₄) δ 9.20 (t, 1H), 7.84 (d, 1H), 6.91-6.78 (m, 3H), 5.86-5.78 (m, 1H), 5.76-5.65 (m, 1H), 3.75-3.63 (m, 1H), 3.41-3.37 (m, 1H), 3.21-3.06 (m, 2H), 3.01-2.94 (m, 3H), 2.93-2.86 (m, 2H), 2.78-2.61 (m, 2H), 2.56-2.44 (m, 2H), 2.41-2.15 (m, 3H), 2.13-2.00 (m, 4H), 1.99-1.92 (m, 4H), 1.89 (d, 3H), 1.85-1.74 (m, 2H), 1.47-1.33 (m, 2H), 1.01-0.94 (m, 6H).

### Trifluoroacetate Salt of Compound 28-2:

LC-Ms m/z (ESI): 734.2 [M+H]⁺

¹H NMR (400 MHz, Methanol-d₄) δ 9.35 (d, 1H), 7.87 (s, 1H), 6.94-6.81 (m, 3H), 5.81 (t, 1H), 5.74-5.65 (m, 1H), 3.75-5.65 (m, 1H), 3.23-3.14 (m, 1H), 3.11-2.98 (m, 4H), 2.94-2.86 (m, 3H), 2.85-2.74 (m, 1H), 2.70-2.61 (m, 1H), 2.52 (t, J=7.4 Hz, 3H), 2.25-2.04 (m, 5H), 2.00 (s, 6H), 1.87-1.76 (m, 4H), 1.41-1.26 (m, 2H), 0.95-0.84 (m, 6H).

### Example 29: Preparation of Compound 29

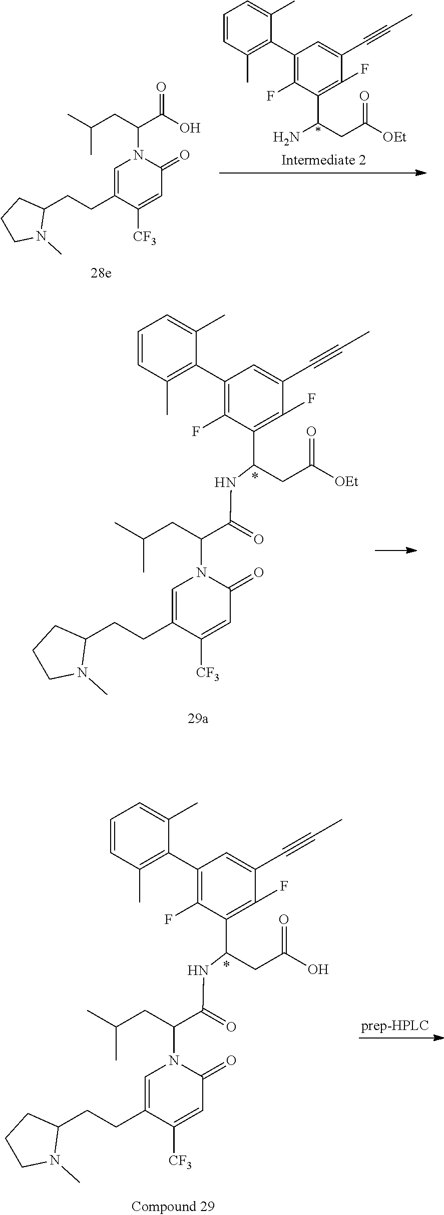 [SMILES: CC(C)CC(C(=O)O)n1cc(CCC2CCCN2C)c(C(F)(F)F)cc1=O ; CC#Cc1cc(-c2c(C)cccc2C)c(F)c(C(N)CC(=O)OCC)c1F ; CC#Cc1cc(-c2c(C)cccc2C)c(F)c(C(CC(=O)OCC)NC(=O)C(CC(C)C)n2cc(CCC3CCCN3C)c(C(F)(F)F)cc2=O)c1F ; CC#Cc1cc(-c2c(C)cccc2C)c(F)c(C(CC(=O)O)NC(=O)C(CC(C)C)n2cc(CCC3CCCN3C)c(C(F)(F)F)cc2=O)c1F]

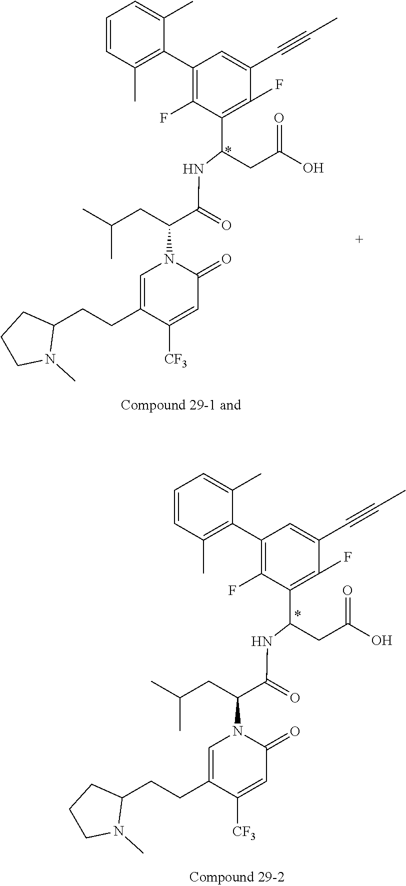 [SMILES: CC#Cc1cc(-c2c(C)cccc2C)c(F)c(C(CC(=O)O)NC(=O)[C@@H](CC(C)C)n2cc(CCC3CCCN3C)c(C(F)(F)F)cc2=O)c1F ; CC#Cc1cc(-c2c(C)cccc2C)c(F)c(C(CC(=O)O)NC(=O)[C@H](CC(C)C)n2cc(CCC3CCCN3C)c(C(F)(F)F)cc2=O)c1F]

Referring to the preparation method of compound 26, crude compound 29 was obtained, which was separated and purified by prep-HPLC (method 2) to afford the trifluoroacetate salt of compound 29-1 (60 mg, Rt=4.181 min) and the trifluoroacetate salt of compound 29-2 (88 mg, Rt=4.279 min).

### Trifluoroacetate Salt of Compound 29-1:

LC-Ms m/z (ESI): 714.2 [M+H]⁺

¹H NMR (400 MHz, Methanol-d₄) δ 9.31 (t, 1H), 7.83 (d, 1H), 7.19 (t, 1H), 7.14-7.03 (m, 3H), 6.83 (s, 1H), 5.86-5.78 (m, 1H), 5.73-5.64 (m, 1H), 3.75-3.64 (m, 1H), 3.22-3.10 (m, 2H), 2.98 (s, 1H), 2.96-2.88 (m, 1H), 2.86 (s, 1H), 2.81-2.51 (m, 3H), 2.36-2.33 (m, 1H), 2.24-2.09 (m, 2H), 2.08-2.01 (m, 5H), 2.01-1.91 (m, 4H), 1.88 (d, 3H), 1.85-1.74 (m, 2H), 1.49-1.31 (m, 2H), 1.01-0.93 (m, 6H).

### Trifluoroacetate Salt of Compound 29-2:

LC-Ms m/z (ESI): 714.2 [M+H]⁺

¹H NMR (400 MHz, Methanol-d₄) δ 9.45 (d, 1H), 7.88 (d, 1H), 7.25-7.09 (m, 4H), 6.88 (d, 1H), 5.86-5.78 (m, 1H), 5.73-5.64 (m, 1H), 3.75-3.64 (m, 1H), 3.43-3.37 (m, 1H), 3.24-3.03 (m, 2H), 3.01-2.90 (m, 4H), 2.86-2.50 (m, 3H), 2.41-2.29 (m, 1H), 2.25-2.11 (m, 2H), 2.11-1.97 (m, 9H), 1.89-1.73 (m, 4H), 1.39-1.26 (m, 2H), 0.95-0.86 (m, 6H).

### Example 30: Preparation of Compound 30 and Isomer Thereof

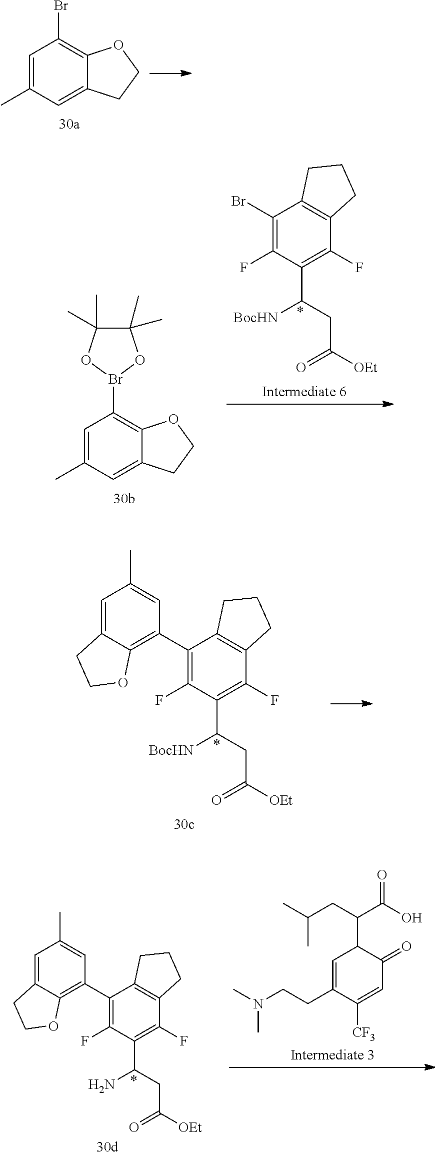

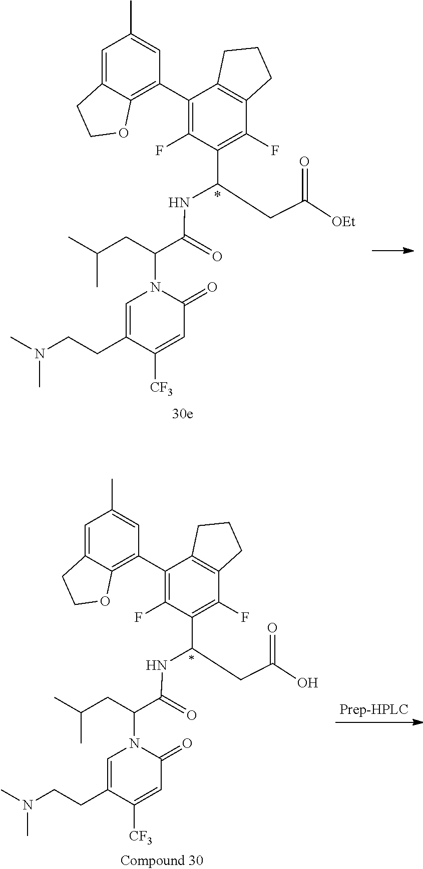 [SMILES: CCOC(=O)CC(NC(=O)C(CC(C)C)n1cc(CCN(C)C)c(C(F)(F)F)cc1=O)c1c(F)c2c(c(-c3cc(C)cc4c3OCC4)c1F)CCC2 ; Cc1cc2c(c(-c3c(F)c(C(CC(=O)O)NC(=O)C(CC(C)C)n4cc(CCN(C)C)c(C(F)(F)F)cc4=O)c(F)c4c3CCC4)c1)OCC2]

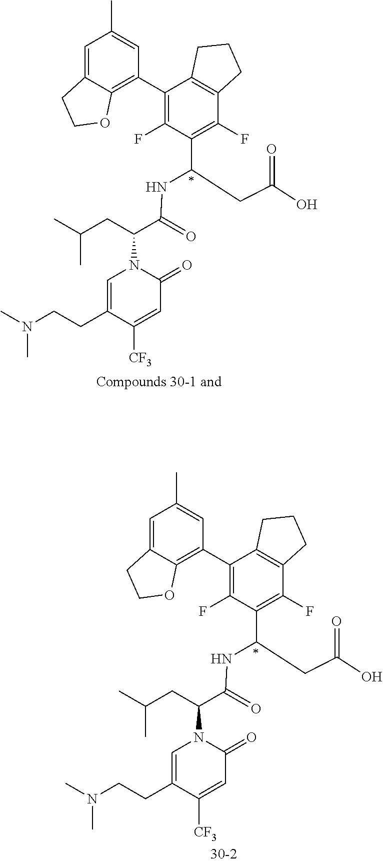 [SMILES: Cc1cc2c(c(-c3c(F)c(C(CC(=O)O)NC(=O)[C@@H](CC(C)C)n4cc(CCN(C)C)c(C(F)(F)F)cc4=O)c(F)c4c3CCC4)c1)OCC2 ; Cc1cc2c(c(-c3c(F)c(C(CC(=O)O)NC(=O)[C@H](CC(C)C)n4cc(CCN(C)C)c(C(F)(F)F)cc4=O)c(F)c4c3CCC4)c1)OCC2]

### Step 1: Synthesis of 30b

Under nitrogen protection, 30a (1.76 g, 8.26 mmol) was dissolved in 1,4-dioxane (60 mL), pinacol diboronate (3.15 g, 12.39 mmol), PdCl2(dppf) (0.60 g, 0.83 mmol) and potassium acetate (2.43 g, 24.78 mmol) were successively added, and the resulting mixture was reacted at 100° C. for 2 h. Extraction was performed by addition of ethyl acetate (180 mL) and the resulting mixture was washed with water (50 mL×2) and saturated sodium chloride aqueous solution (50 mL×1), dried over anhydrous sodium sulfate and filtered and the filtrate was concentrated under reduced pressure and subjected to column chromatography on a silica gel column to afford 22b (1.35 g, yield: 62.86%).

Referring to the preparation method of compound 20, and taking 30b and intermediate 6 as starting materials, crude compound 30 was obtained, which was separated and purified by prep-HPLC (method 2) to afford the trifluoroacetate salt of compound 30-1 (10 mg, Rt=4.121 min, yield: 10.4%) and the trifluoroacetate salt of compound 30-2 (10 mg, Rt 4.175 min, yield: 10.4%).

### Trifluoroacetate Salt of Compound 30-1:

LC-Ms m/z (ESI): 704.3 [M+H]⁺

¹H NMR (400 MHz, CD₃OD) δ 7.90 (d, 1H), 7.04 (s, 1H), 6.84 (s, 1H), 6.76 (d, 1H), 5.85-5.76 (m, 1H), 5.75-5.65 (m, 1H), 4.52-4.42 (m, 2H), 3.30-3.25 (m, 1H), 3.24-3.15 (m, 2H), 3.05-3.05 (m, 1H), 3.05-2.87 (m, 10H), 2.87-2.74 (m, 2H), 2.69-2.55 (m, 1H), 2.30-2.22 (m, 3H), 2.11-1.86 (m, 4H), 1.47-1.33 (m, 1H), 1.00-0.89 (m, 6H).

### Trifluoroacetate Salt of Compound 30-2:

LC-Ms m/z (ESI): 704.3 [M+H]⁺

¹H NMR (400 MHz, CD₃OD) δ 7.90 (s, 1H), 7.06 (s, 1H), 6.88 (s, 1H), 6.77 (d, 1H), 5.84-5.74 (m, 1H), 5.73-5.61 (m, 1H), 4.54-4.44 (m, 2H), 3.37-3.31 (m, 1H), 3.26-3.18 (m, 2H), 3.14-2.91 (m, 11H), 2.90-2.75 (m, 2H), 2.73-2.54 (m, 1H), 2.32-2.24 (m, 3H), 2.14-2.00 (m, 2H), 1.87-1.77 (m, 2H), 1.38-1.24 (m, 1H), 0.98-0.83 (m, 6H).

### Example 32: Preparation of Compound 32 and Isomer Thereof

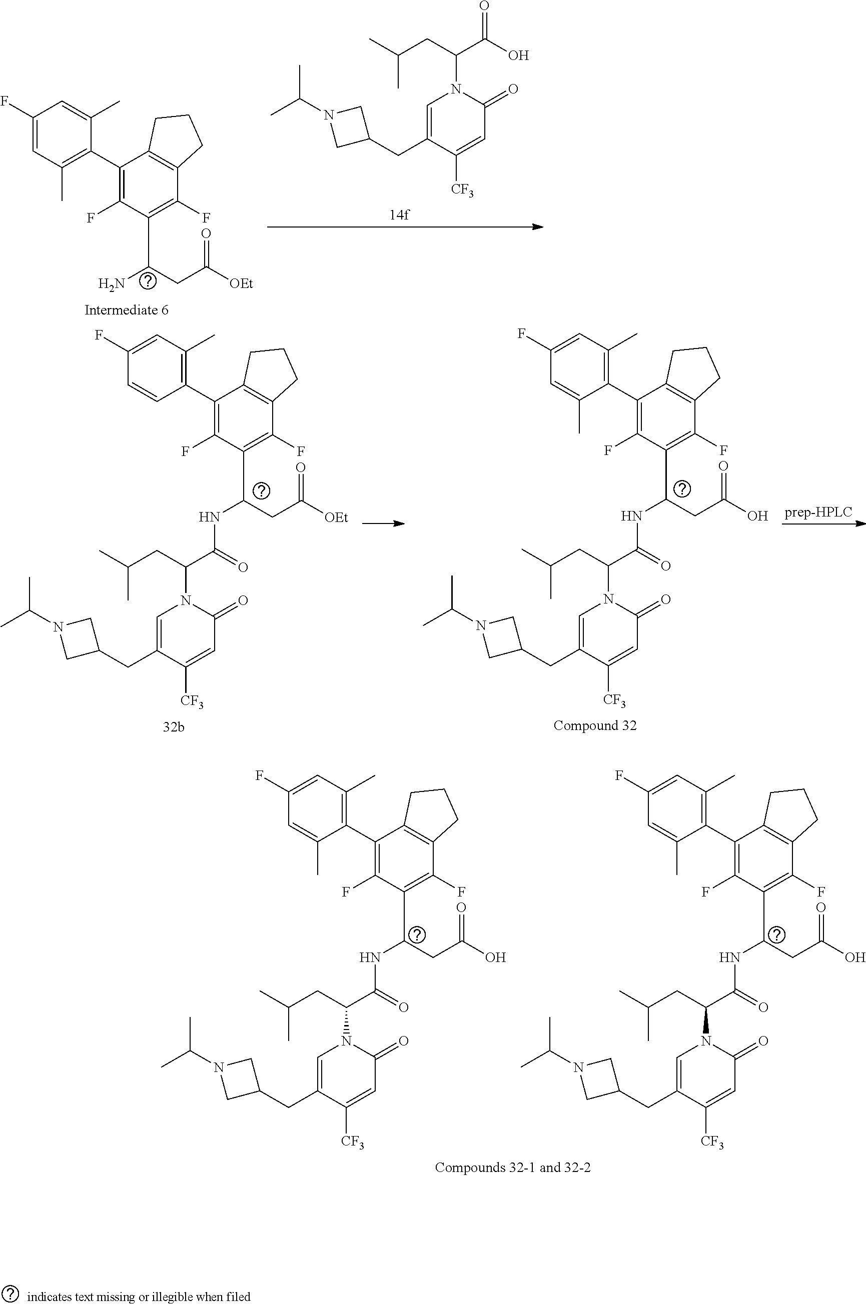

Referring to the preparation method of Example 21, crude compound 32 was obtained, which was separated and purified by prep-HPLC (method 2) to afford the trifluoroacetate salt of compound 32-1 (19 mg, Rt=4.790 min, yield: 8.93%) and the trifluoroacetate salt of compound 32-2 (20 mg, Rt=4.862 min, yield: 8.46%).

### Trifluoroacetate Salt of Compound 32-1:

LC-Ms m/z (ESI): 734.8 [M+H]⁺

¹H NMR (400 MHz, CD₃OD) δ 9.23-9.07 (m, 1H), 7.82 (s, 1H), 6.89-6.78 (m, 3H), 5.85-5.76 (m, 1H), 5.74-5.64 (m, 1H), 4.33-3.83 (m, 4H), 3.51-3.35 (m, 1H), 3.18-2.81 (m, 7H), 2.50-2.39 (m, 2H), 2.13-1.99 (m, 3H), 1.94-1.83 (m, 6H), 1.40-1.29 (m, 2H), 1.27-1.16 (m, 6H), 1.02-0.88 (m, 6H).

### Trifluoroacetate Salt of Compound 32-2:

LC-Ms m/z (ESI): 734.8 [M+H]⁺

¹H NMR (400 MHz, CD₃OD) δ 7.94-7.73 (m, 1H), 6.93-6.83 (m, 3H), 5.84-5.65 (m, 2H), 4.26-4.07 (m, 2H), 4.00-3.83 (m, 2H), 3.46-3.35 (m, 1H), 3.19-2.77 (m, 7H), 2.53-2.43 (m, 2H), 2.16-2.03 (m, 2H), 1.98 (s, 6H), 1.87-1.70 (m, 2H), 1.38-1.18 (m, 7H), 0.97-0.81 (m, 6H).

### Example 33: Preparation of Compound 33 and Isomer Thereof

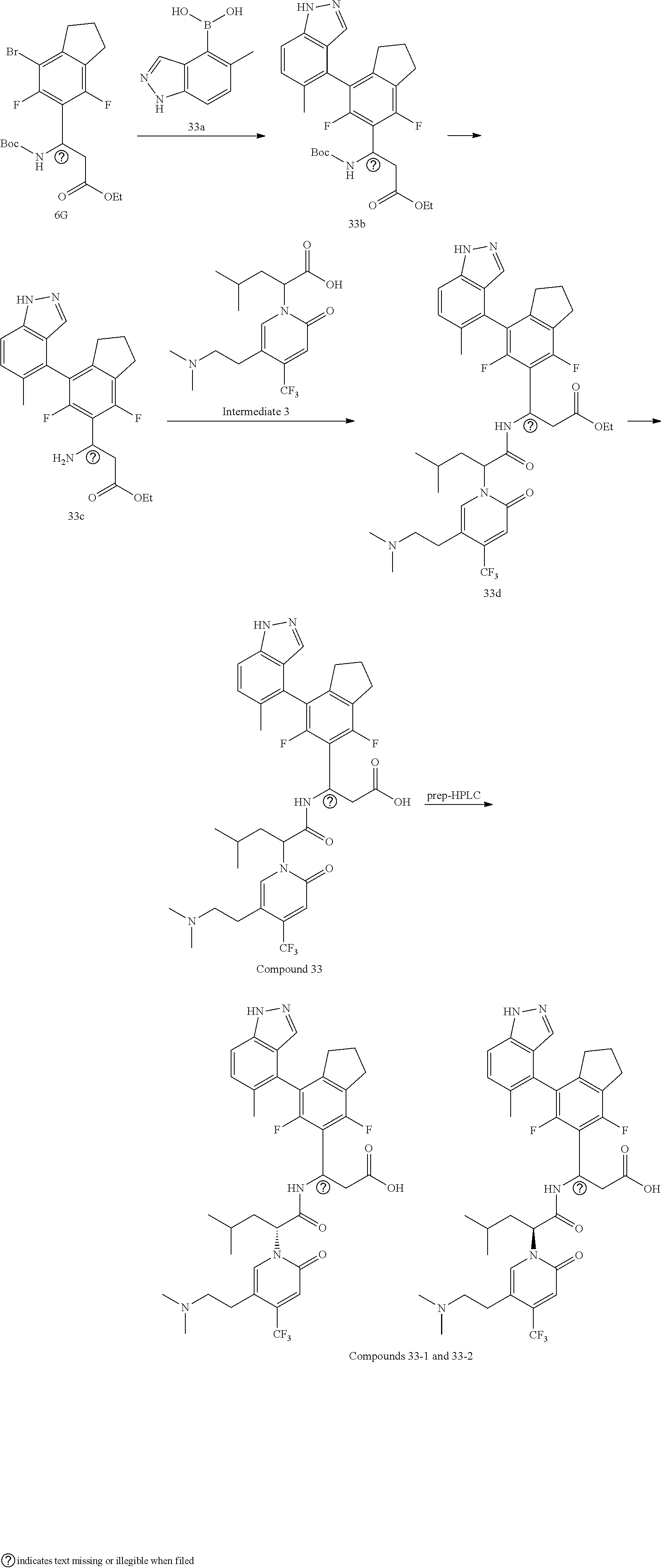

Referring to the preparation method of Example 21, crude compound 33 was obtained, which was separated and purified by prep-HPLC (method 1) to afford compound 33-1 (20 mg, Rt=3.714 min, yield: 7.50%) and compound 33-2 (25 mg, Rt=3.765 min, yield: 9.38%).

### Compound 33-1:

LC-Ms m/z (ESI): 702.7 [M+H]⁺

¹H NMR (400 MHz, CD₃OD) δ 7.84 (d, 1H), 7.52-7.42 (m, 2H), 7.36-7.30 (m, 1H), 6.87-6.80 (m, 1H), 5.76-5.61 (m, 2H), 3.05-2.83 (m, 7H), 2.76-2.58 (m, 7H), 2.54-2.41 (m, 2H), 2.19-1.91 (m, 7H), 1.43-1.31 (m, 1H), 0.97-0.85 (m, 6H).

### Compound 33-2:

LC-Ms m/z (ESI): 702.7 [M+H]⁺

¹H NMR (400 MHz, CD₃OD) δ 7.83-7.78 (m, 1H), 7.53-7.45 (m, 2H), 7.40-7.33 (m, 1H), 6.92-6.87 (m, 1H), 5.87-5.79 (m, 1H), 5.68-5.58 (m, 1H), 3.28-3.14 (m, 2H), 3.06-2.94 (m, 4H), 2.85-2.75 (m, 7H), 2.62-2.42 (m, 3H), 2.21-2.16 (m, 3H), 2.13-2.02 (m, 2H), 1.97-1.85 (m, 1H), 1.81-1.70 (m, 1H), 1.40-1.27 (m, 1H), 0.94-0.81 (m, 6H).

### Example 35: Preparation of Compound 35

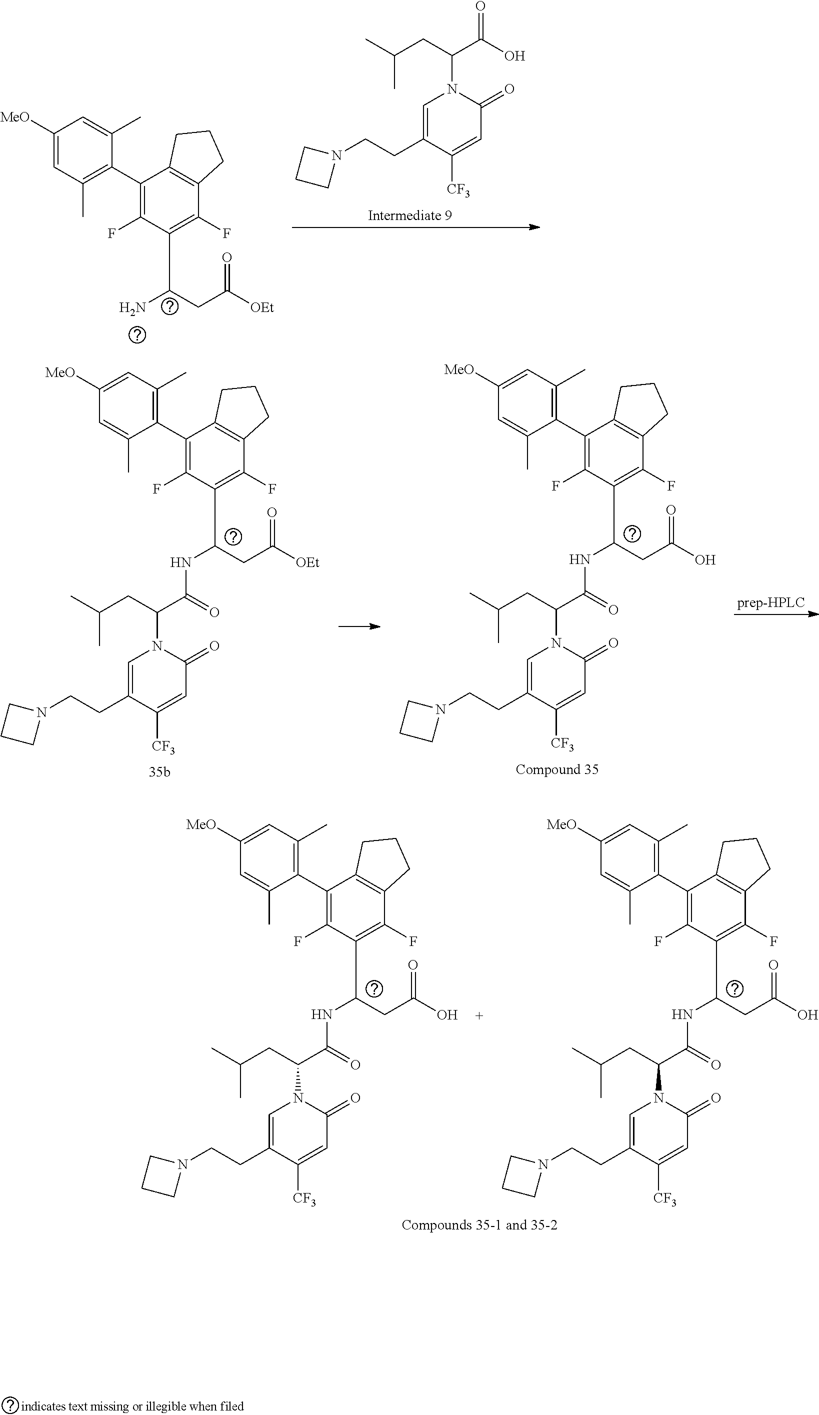 [SMILES: CCOC(=O)CC(N)c1c(F)c2c(c(-c3c(C)cc(OC)cc3C)c1F)CCC2 ; CC(C)CC(C(=O)O)n1cc(CCN2CCC2)c(C(F)(F)F)cc1=O ; CCOC(=O)CC(NC(=O)C(CC(C)C)n1cc(CCN2CCC2)c(C(F)(F)F)cc1=O)c1c(F)c2c(c(-c3c(C)cc(OC)cc3C)c1F)CCC2 ; COc1cc(C)c(-c2c(F)c(C(CC(=O)O)NC(=O)C(CC(C)C)n3cc(CCN4CCC4)c(C(F)(F)F)cc3=O)c(F)c3c2CCC3)c(C)c1 ; COc1cc(C)c(-c2c(F)c(C(CC(=O)O)NC(=O)[C@@H](CC(C)C)n3cc(CCN4CCC4)c(C(F)(F)F)cc3=O)c(F)c3c2CCC3)c(C)c1 ; COc1cc(C)c(-c2c(F)c(C(CC(=O)O)NC(=O)[C@H](CC(C)C)n3cc(CCN4CCC4)c(C(F)(F)F)cc3=O)c(F)c3c2CCC3)c(C)c1]

### Step 1: Synthesis of 35b

Under nitrogen protection, intermediate 9 (referring to WO 2021076902 for synthesis) (390 mg, 1.08 mmol) was dissolved in dry DMF (5 mL), and HATU (750 mg, 1.97 mmol) and DIPEA (510 mg, 3.98 mmol) were successively added. At room temperature, the reaction mixture was stirred for 40 min, and then intermediate 21a (400 mg, 0.99 mmol) was added. The resulting mixture was reacted at room temperature overnight. Ethyl acetate (80 mL) was added and the resulting mixture was successively washed with water (20 mL×2) and saturated sodium chloride aqueous solution (20 mL×1), dried over anhydrous sodium sulfate and filtered and the filtrate was concentrated under reduced pressure and subjected to column chromatography on a silica gel column to afford 35b (500 mg, yield: 67.72%).

LC-Ms m/z (ESI): 746.8[M+H]⁺

### Step 2: Synthesis of Compound 35-1 and Compound 35-2

35b (500 mg, 0.67 mmol) was dissolved in THF (9 mL) and water (3 mL), lithium hydroxide monohydrate (40 mg, 1.68 mmol) was added, and the resulting mixture was reacted at room temperature for 30 min. PH was adjusted to 5-6 with 1 N hydrochloric acid, and the resulting solution was concentrated under reduced pressure to afford crude compound 35, which was separated and purified by prep-HPLC (method 1) to afford compound 35-1 (21 mg, Rt=4.109 min, yield: 4.37%) and compound 35-2 (22 mg, Rt=4.204 min, yield: 4.57%).

### Compound 35-1:

LC-Ms m/z (ESI): 718.7 [M+H]⁺

¹H NMR (400 MHz, CD₃OD) δ 7.77 (s, 1H), 6.88 (s, 1H), 6.68 (s, 2H), 5.75-5.68 (m, 1H), 5.68-5.60 (m, 1H), 4.06-3.95 (m, 4H), 3.78 (s, 3H), 3.00-2.82 (m, 5H), 2.69-2.59 (m, 1H), 2.51-2.37 (m, 4H), 2.11-2.01 (m, 2H), 2.00-1.86 (s, 9H), 1.46-1.34 (m, 1H), 0.97-0.86 (m, 6H).

### Compound 35-2:

LC-Ms m/z (ESI): 718.7 [M+H]⁺

¹H NMR (400 MHz, CD₃OD) δ 7.66 (s, 1H), 6.92 (s, 1H), 6.70 (s, 2H), 5.92-5.85 (m, 1H), 5.63-5.56 (m, 1H), 4.17-4.06 (m, 4H), 3.79 (s, 3H), 3.46-3.32 (m, 2H), 3.02-2.88 (m, 3H), 2.87-2.72 (m, 2H), 2.53-2.39 (m, 5H), 2.14-2.03 (m, 2H), 2.00-1.90 (m, 7H), 1.77-1.68 (m, 1H), 1.43-1.31 (m, 1H), 0.93-0.84 (m, 6H).

### Example 36: Preparation of Compound 36

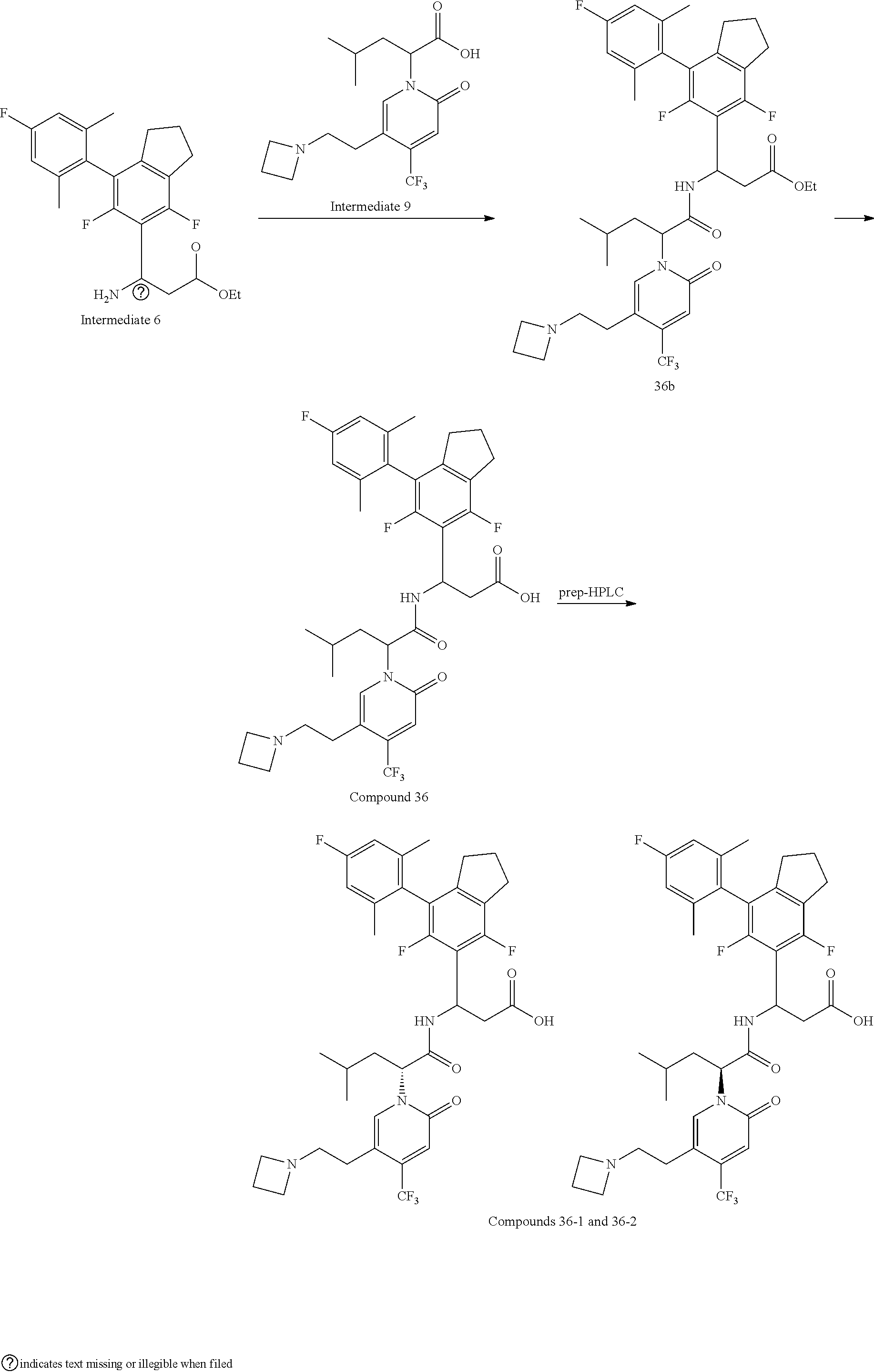 [SMILES: CCOC(O)CC(N)c1c(F)c2c(c(-c3c(C)cc(F)cc3C)c1F)CCC2 ; CC(C)CC(C(=O)O)n1cc(CCN2CCC2)c(C(F)(F)F)cc1=O ; CCOC(=O)CC(NC(=O)C(CC(C)C)n1cc(CCN2CCC2)c(C(F)(F)F)cc1=O)c1c(F)c2c(c(-c3c(C)cc(F)cc3C)c1F)CCC2 ; Cc1cc(F)cc(C)c1-c1c(F)c(C(CC(=O)O)NC(=O)C(CC(C)C)n2cc(CCN3CCC3)c(C(F)(F)F)cc2=O)c(F)c2c1CCC2 ; Cc1cc(F)cc(C)c1-c1c(F)c(C(CC(=O)O)NC(=O)[C@@H](CC(C)C)n2cc(CCN3CCC3)c(C(F)(F)F)cc2=O)c(F)c2c1CCC2 ; Cc1cc(F)cc(C)c1-c1c(F)c(C(CC(=O)O)NC(=O)[C@H](CC(C)C)n2cc(CCN3CCC3)c(C(F)(F)F)cc2=O)c(F)c2c1CCC2]

Referring to the preparation method of Example 35, crude compound 36 was obtained, which was separated and purified by prep-HPLC (method 1) to afford compound 36-1 (19 mg, Rt=5.540 min, yield: 6.57%) and compound 36-2 (21 mg, Rt=5.599 min, yield: 7.26%).

### Compound 36-1:

LC-Ms m/z (ESI): 706.7 [M+H]⁺

¹H NMR (400 MHz, CD₃OD) δ 7.86 (s, 1H), 6.91-6.78 (m, 3H), 5.83-5.66 (m, 2H), 4.43-4.01 (m, 4H), 3.45-3.33 (m, 2H), 3.15-3.04 (m, 1H), 2.99-2.76 (m, 5H), 2.68-2.54 (m, 1H), 2.53-2.37 (m, 3H), 2.11-1.99 (m, 3H), 1.97-1.83 (m, 7H), 1.47-1.33 (m, 1H), 1.00-0.89 (m, 6H).

### Compound 36-2:

LC-Ms m/z (ESI): 706.7 [M+H]⁺

¹H NMR (400 MHz, CD₃OD) δ 7.67 (s, 1H), 6.95-6.84 (m, 3H), 5.94-5.85 (m, 1H), 5.64-5.55 (m, 1H), 4.17-4.04 (m, 4H), 3.46-3.33 (m, 2H), 3.04-2.71 (m, 5H), 2.53-2.39 (m, 5H), 2.16-2.03 (m, 2H), 2.01-1.87 (m, 7H), 1.77-1.67 (m, 1H), 1.42-1.30 (m, 1H), 0.93-0.82 (m, 6H).

### Example 37: Preparation of Compound 37

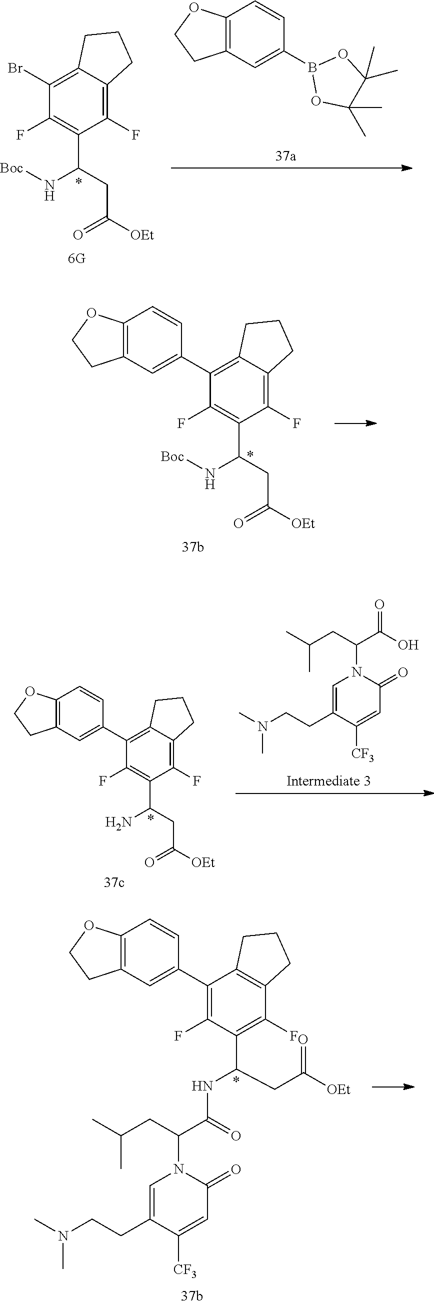

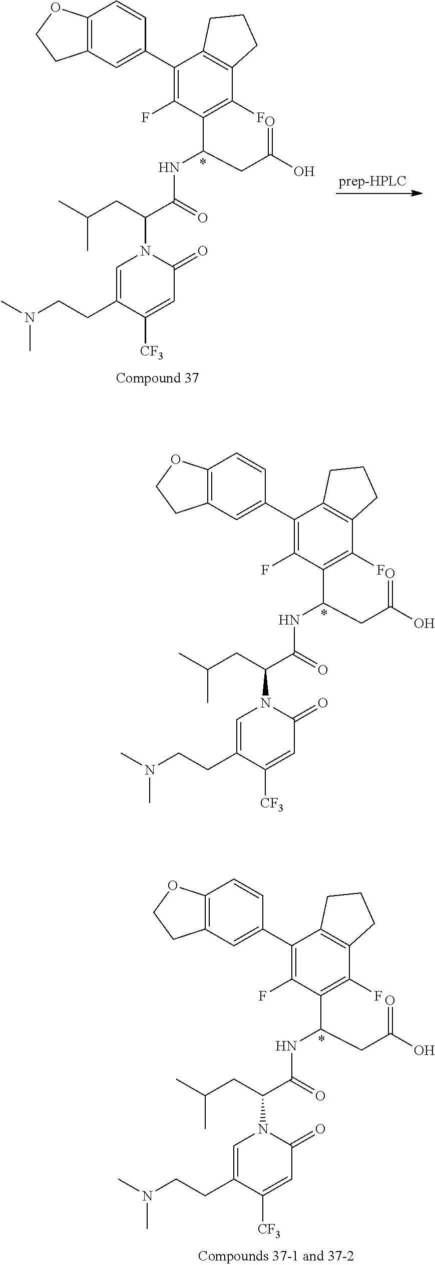 [SMILES: CC(C)CC(C(=O)NC(CC(=O)O)c1c(F)c2c(c(-c3ccc4c(c3)CCO4)c1F)CCC2)n1cc(CCN(C)C)c(C(F)(F)F)cc1=O ; CC(C)C[C@@H](C(=O)NC(CC(=O)O)c1c(F)c2c(c(-c3ccc4c(c3)CCO4)c1F)CCC2)n1cc(CCN(C)C)c(C(F)(F)F)cc1=O ; CC(C)C[C@H](C(=O)NC(CC(=O)O)c1c(F)c2c(c(-c3ccc4c(c3)CCO4)c1F)CCC2)n1cc(CCN(C)C)c(C(F)(F)F)cc1=O]

Referring to the preparation method of Example 20, crude compound 37 was obtained, which was separated and purified by prep-HPLC (method 1) to afford compound 37-1 (18 mg, Rt=5.359 min, yield: 6.21%) and compound 37-2 (19 mg, Rt=5.401 min, yield: 6.56%).

### Compound 37-1:

LC-Ms m/z (ESI): 690.7 [M+H]⁺

¹H NMR (400 MHz, CD₃OD) δ 7.86 (s, 1H), 7.12 (s, 1H), 6.98 (d, 1H), 6.83 (s, 1H), 6.73 (d, 1H), 5.74-5.64 (m, 2H), 4.56 (t, 2H), 3.26-3.17 (m, 2H), 3.12-3.02 (m, 2H), 2.98-2.84 (m, 5H), 2.79-2.65 (m, 9H), 2.08-1.94 (m, 4H), 1.46-1.35 (m, 1H), 0.97-0.91 (m, 6H).

### Compound 37-2:

LC-Ms m/z (ESI): 690.7 [M+H]⁺

¹H NMR (400 MHz, CD₃OD) δ 7.80 (s, 1H), 7.16 (s, 1H), 7.02 (d, 1H), 6.91 (s, 1H), 6.77 (d, 1H), 5.85-5.77 (m, 1H), 5.69-5.60 (m, 1H), 4.58 (t, 2H), 3.29-3.12 (m, 4H), 3.05-2.89 (m, 4H), 2.83-2.72 (m, 9H), 2.57-2.48 (m, 1H), 2.11-2.00 (m, 2H), 1.96-1.88 (m, 1H), 1.81-1.70 (m, 1H), 1.41-1.28 (m, 1H), 0.93-0.82 (m, 6H).

### Example 38: Preparation of Compound 38

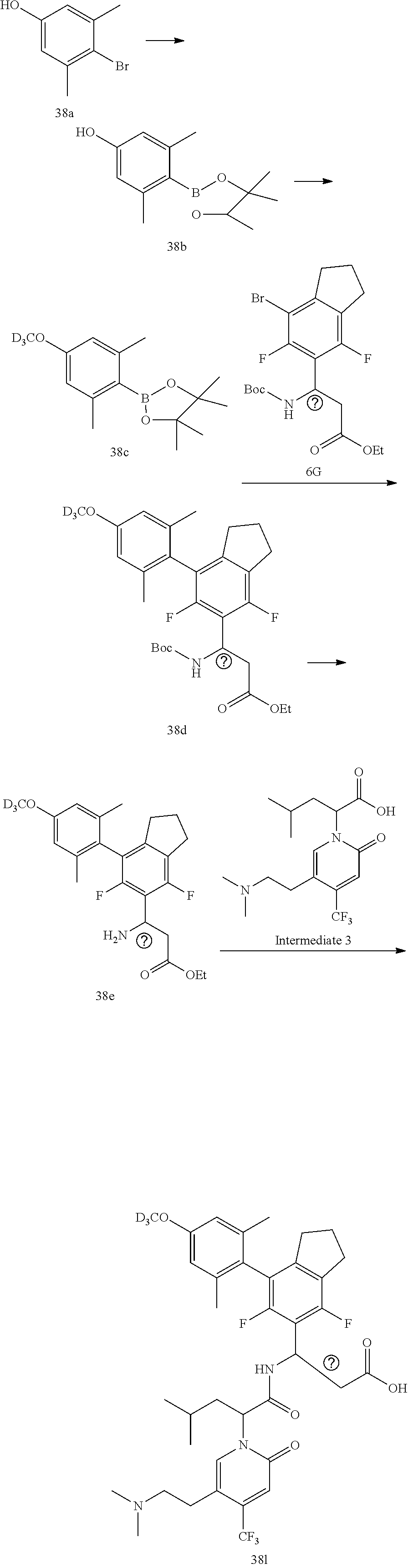

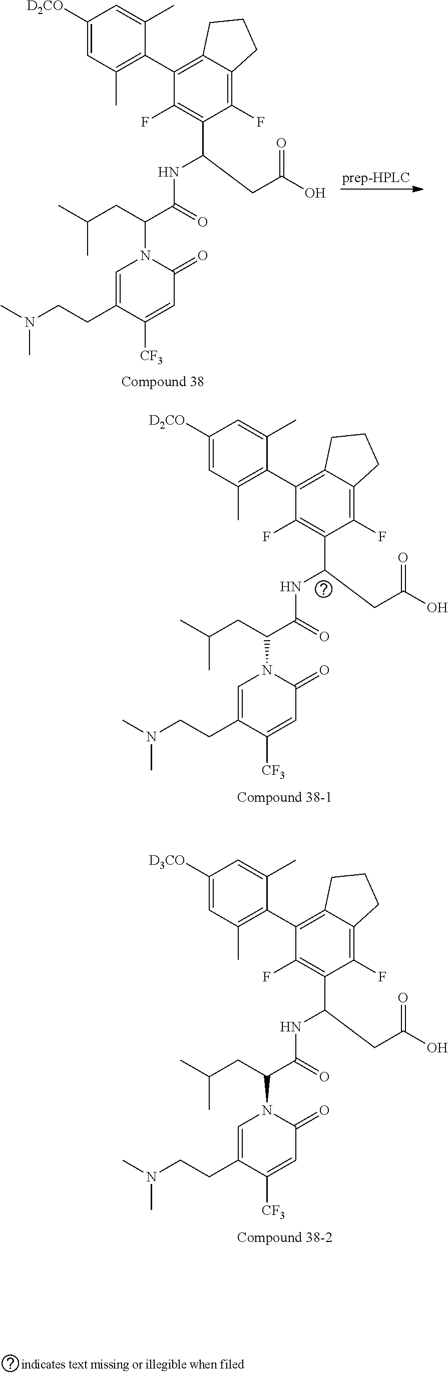

### Step 1: Synthesis of 38b

Under nitrogen protection, 38a (3 g, 14.91 mmol), pinacol diboronate (4.54 g, 17.88 mmol), potassium carbonate (2.06 g, 14.91 mmol) and PdCl₂(dppf)·DCM (CAS: 95464-05-4) (1.22 g, 1.49 mmol) were dissolved in 1.4-dioxane (30 mL) and water (3 mL), and the mixture was refluxed and reacted at 100° C. overnight. Water (20 mL) was added, and the reaction solution was extracted with ethyl acetate (30 ml×3), washed with saturated sodium chloride aqueous solution (20 mL×2), dried over anhydrous sodium sulfate, filtered, concentrated under reduced pressure, and then subjected to flash column chromatography to afford 38b (2.8 g, yield: 75.68%).

### Step 2: Synthesis of 38c

38b (2.8 g, 11.28 mmol) was dissolved in a solution of dichloromethane (20 mL), and NaH (0.54 g, 22.5 mmol) was added slowly in portions under ice bath conditions. After stirring for 20 minutes, deuterated iodomethane (3.27 g, 22.56 mmol) was added, and the reaction was carried out overnight. Water (20 mL) was added and the resulting mixture was extracted with ethyl acetate (30 ml×3), washed with saturated sodium chloride aqueous solution (20 mL×2), dried over anhydrous sodium sulfate, filtered and concentrated under reduced pressure. The residue was subjected to flash column chromatography to afford 38c (1.9 g, yield: 63.52%).

For step 3 to step 6, referring to the preparation method of Example 21, crude compound 38 was obtained, which was separated and purified by prep-HPLC (method 1) to afford compound 38-1 (20 mg, Rt=4.048 min, yield: 9.41%) and compound 38-2 (25 mg, Rt=4.301 min, yield: 11.76%).

### Compound 38-1:

LC-Ms m/z (ESI): 709.7 [M+H]⁺

¹H NMR (400 MHz, CD₃OD) δ 7.83 (s, 1H), 6.82 (s, 1H), 6.68-6.62 (m, 2H), 5.74-5.63 (m, 2H), 3.00-2.83 (m, 7H), 2.71-2.58 (m, 7H), 2.48-2.39 (m, 2H), 2.08-1.93 (m, 4H), 1.91 (s, 3H), 1.83 (s, 3H), 1.46-1.34 (m, 1H), 0.97-0.88 (m, 6H).

### Compound 38-2:

LC-Ms m/z (ESI): 709.7 [M+H]⁺

¹H NMR (400 MHz, CD₃OD) δ 7.85 (s, 1H), 6.90 (s, 1H), 6.69 (s, 2H), 5.81-5.74 (m, 1H), 5.71-5.64 (m, 1H), 3.29-3.18 (m, 2H), 3.06-2.82 (m, 11H), 2.73-2.62 (m, 1H), 2.53-2.43 (m, 2H), 2.13-2.01 (m, 2H), 1.94 (s, 6H), 1.91-1.82 (m, 1H), 1.82-1.72 (m, 1H), 1.41-1.26 (m, 1H), 0.94-0.82 (m, 6H).

### Example 39: Preparation of Compound 39

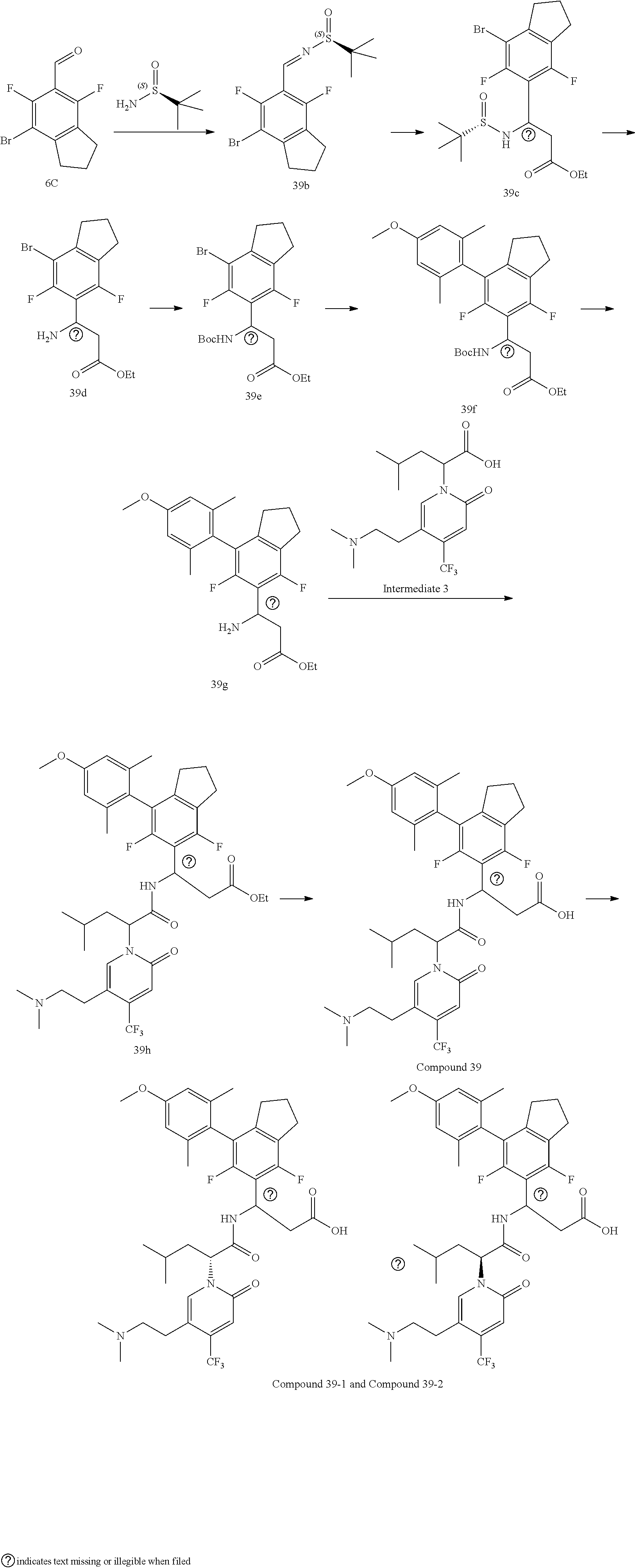

Referring to the preparation method of intermediate 6 and compound 21, crude compound 39 was obtained, which was separated and purified by prep-HPLC (method 1) to afford compound 39-1 (28 mg, Rt=4.070 min) and compound 39-2 (30 mg, Rt=4.143 min).

### Compound 39-1:

LC-Ms m/z (ESI): 706.7 [M+H]⁺

¹H NMR (400 MHz, CD₃OD) δ 7.85 (s, 1H), 6.83 (s, 1H), 6.67 (s, 2H), 5.72-5.63 (m, 2H), 3.78 (s, 3H), 3.117-2.99 (s, 2H), 2.98-2.86 (m, 5H), 2.75-2.62 (m, 7H), 2.48-2.40 (m, 2H), 2.09-2.00 (m, 2H), 1.99-1.94 (m, 2H), 1.92 (s, 3H), 1.85 (s, 3H), 1.47-1.33 (m, 1H), 0.99-0.87 (in, 6H).

### Compound 39-2:

LC-Ms m/z (ESI): 706.7 [M+H]⁺

¹H NMR (400 MHz, CD₃OD) δ 7.80 (s, 1H), 6.90 (s, 1H), 6.69 (s, 2H), 5.86-5.79 (m, 1H), 5.65-5.59 (m, 1H), 3.79 (s, 3H), 3.28-3.10 (m, 2H), 3.02-2.93 (m, 4H), 2.83-2.72 (m, 7H), 2.58-2.44 (m, 3H), 2.13-2.01 (m, 2H), 1.97-1.87 (m, 7H), 1.79-1.70 (m, 1H), 1.41-1.29 (m, 1H), 0.93-0.83 (m, 6H).

### Example 40: Preparation of Compound 40

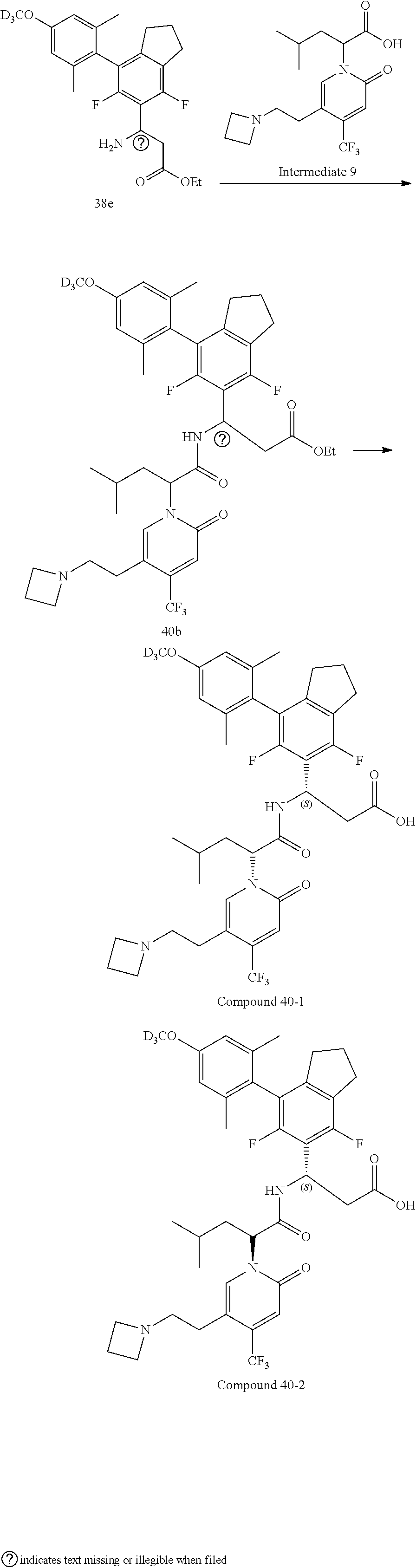

Referring to the preparation method of compound 38, crude compound 40 was obtained, which was separated and purified by prep-HPLC (method 1) to afford compound 40-1 (11 mg, Rt=4.152 min) and compound 40-2 (10 mg, Rt=4.238 min).

### Compound 40-1:

LC-Ms m/z (ESI): 721.7 [M+H]⁺

¹H NMR (400 MHz, CD₃OD) δ 7.76 (s, 1H), 6.88 (s, 1H), 6.67 (s, 2H), 5.75-5.68 (m, 1H), 5.67-5.59 (m, 1H), 4.03-3.90 (m, 4H), 3.35-3.22 (m, 2H), 3.00-2.92 (m, 2H), 2.92-2.80 (m, 3H), 2.67-2.59 (m, 1H), 2.51-2.36 (m, 4H), 2.11-2.00 (m, 2H), 1.99-1.92 (m, 5H), 1.89 (s, 3H), 1.47-1.35 (m, 1H), 0.98-0.87 (m, 6H).

### Compound 40-2:

LC-Ms m/z (ESI): 721.7 [M+H]⁺

¹H NMR (400 MHz, CD₃OD) δ 7.66 (s, 1H), 6.92 (s, 1H), 6.69 (s, 2H), 5.89 (dd, 1H), 5.59 (t, 1H), 4.16-4.03 (s, 4H), 3.45-3.33 (m, 2H), 3.03-2.70 (m, 5H), 2.53-2.38 (m, 5H), 2.13-2.03 (m, 2H), 2.00-1.88 (m, 7H), 1.79-1.68 (m, 1H), 1.43-1.33 (m, 1H), 0.95-0.83 (m, 6H).

### Example 41: Preparation of Compound 41

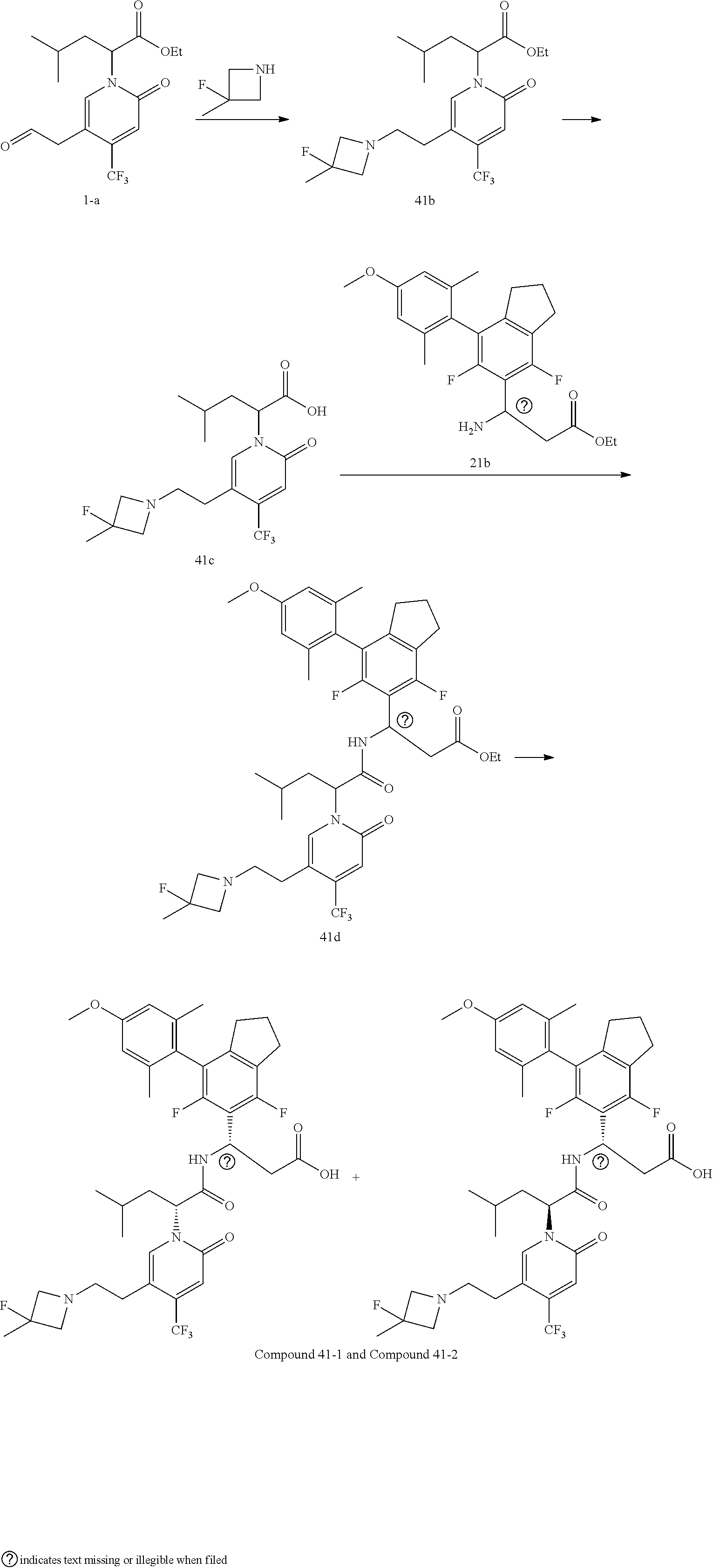

Referring to the preparation method of compound 9, crude compound 41 was obtained, which was separated and purified by prep-HPLC (method 1) to afford compound 41-1 (20 mg, Rt=4.286 min) and compound 41-2 (25 mg, Rt=4.371 min).

### Compound 41-1:

LC-Ms m/z (ESI): 750.3 [M+H]⁺

¹H NMR (400 MHz, CD₃OD) δ 7.80 (s, 1H), 6.80 (s, 1H), 6.69-6.63 (m, 2H), 5.74-5.66 (m, 2H), 3.78 (s, 3H), 3.73-3.47 (m, 4H), 3.05-2.87 (m, 5H), 2.82-2.74 (m, 1H), 2.73-2.60 (m, 2H), 2.48-2.40 (m, 2H), 2.09-1.98 (m, 2H), 1.98-1.88 (m, 5H), 1.83 (s, 3H), 1.63-1.52 (m, 3H), 1.47-1.36 (m, 1H), 0.99-0.90 (m, 6H).

### Compound 41-2:

LC-Ms m/z (ESI): 750.3 [M+H]⁺

¹H NMR (400 MHz, CD₃OD) δ 7.74 (s, 1H), 6.88 (s, 1H), 6.69 (s, 2H), 5.84-5.76 (m, 1H), 5.70-5.62 (m, 1H), 4.03-3.75 (m, 7H), 3.20-3.13 (m, 2H), 3.02-2.94 (m, 2H), 2.91-2.70 (m, 3H), 2.68-2.59 (m, 1H), 2.53-2.45 (m, 2H), 2.13-2.02 (m, 2H), 1.97-1.83 (m, 7H), 1.81-1.71 (m, 1H), 1.66-1.55 (m, 3H), 1.40-1.29 (m, 1H), 0.95-0.83 (m, 6H).

### Example 42: Preparation of Compound 42

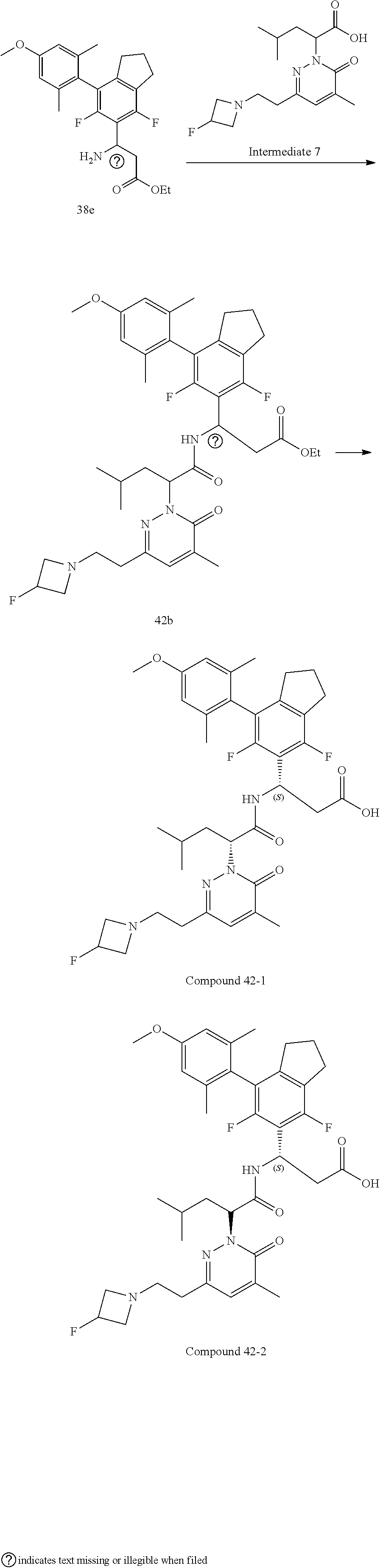

Referring to the preparation method of compound 35, crude compound 42 was obtained, which was separated and purified by prep-HPLC (method 1) to afford compound 42-1 (15 mg, Rt=4.071 min) and compound 42-2 (18 mg, Rt=4.228 min).

### Compound 42-1:

LC-Ms m/z (ESI): 683.3 [M+H]⁺

¹H NMR (400 MHz, CD₃OD) δ 7.29 (s, 1H), 6.69 (s, 2H), 5.56 (dd, 1H), 5.49 (t, 1H), 5.34-5.12 (m, 1H), 4.32-4.08 (m, 2H), 3.93-3.82 (m, 1H), 3.80-3.69 (m, 4H), 3.49-3.33 (m, 2H), 3.00-2.85 (m, 3H), 2.85-2.75 (m, 1H), 2.66-2.43 (m, 4H), 2.24-2.14 (m, 4H), 2.12-2.02 (m, 2H), 1.96-1.90 (m, 6H), 1.79-1.70 (m, 1H), 1.41-1.28 (m, 1H), 0.91-0.83 (m, 6H).

### Compound 42-2:

LC-Ms m/z (ESI): 683.3 [M+H]⁺

¹H NMR (400 MHz, CD₃OD) δ 7.28-7.24 (m, 1H), 6.69 (s, 2H), 5.73 (dd, 1H), 5.66-5.59 (m, 1H), 5.40-5.17 (m, 1H), 4.43-4.26 (m, 2H), 4.10-3.88 (m, 2H), 3.79 (s, 3H), 3.49-3.40 (m, 2H), 3.02-2.83 (m, 4H), 2.70-2.60 (m, 1H), 2.55-2.44 (m, 3H), 2.21-2.15 (m, 3H), 2.14-2.02 (m, 3H), 1.96-1.91 (m, 6H), 1.88-1.78 (m, 1H), 1.41-1.27 (m, 1H), 0.91-0.82 (m, 6H).

### Example 43: Preparation of Compound 43

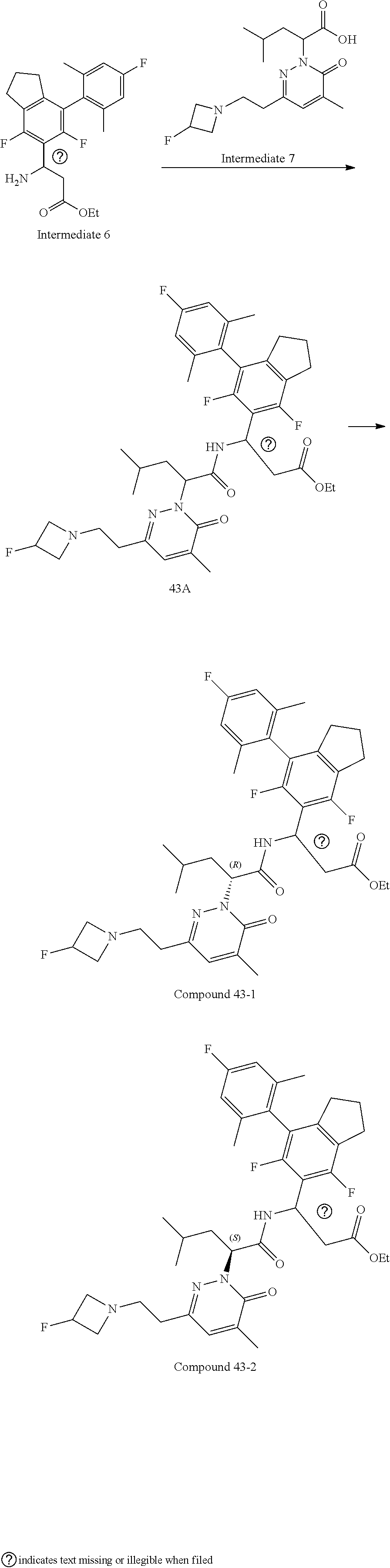

Referring to the preparation method of compound 35, crude compound 42 was obtained, which was separated and purified by prep-HPLC (method 1) to afford compound 43-1 (98 mg, Rt=4.126 min) and compound 43-2 (103 mg, Rt=4.303 min).

### Compound 43-1:

LC-Ms m/z (ESI): 671.3 [M+H]⁺

¹H NMR (400 MHz, CD₃OD) δ 7.32-7.27 (m, 1H), 6.92-6.82 (m, 2H), 5.60-5.47 (m, 2H), 5.33-5.08 (m, 1H), 4.19-3.96 (m, 2H), 3.81-3.61 (m, 2H), 3.37-3.23 (m, 2H), 3.02-2.92 (m, 2H), 2.90-2.70 (m, 2H), 2.67-2.54 (m, 2H), 2.52-2.41 (m, 2H), 2.24-2.13 (m, 4H), 2.13-2.02 (m, 2H), 1.99-1.94 (m, 6H), 1.80-1.70 (m, 1H), 1.42-1.26 (m, 1H), 0.92-0.81 (m, 6H).

### Compound 43-2:

LC-Ms m/z (ESI): 671.3 [M+H]⁺

¹H NMR (400 MHz, CD₃OD) δ 7.29-7.25 (m, 1H), 6.90-6.84 (m, 2H), 5.76-5.69 (m, 1H), 5.65-5.58 (m, 1H), 5.41-5.19 (m, 1H), 4.44-4.31 (m, 2H), 4.12-3.92 (m, 2H), 3.50-3.44 (m, 2H), 3.02-2.84 (m, 4H), 2.70-2.61 (m, 1H), 2.54-2.45 (m, 3H), 2.20-2.16 (m, 3H), 2.14-2.03 (m, 3H), 1.99-1.93 (m, 6H), 1.88-1.79 (m, 1H), 1.40-1.27 (m, 1H), 0.90-0.83 (m, 6H).

### Example 44: Preparation of Compound 44

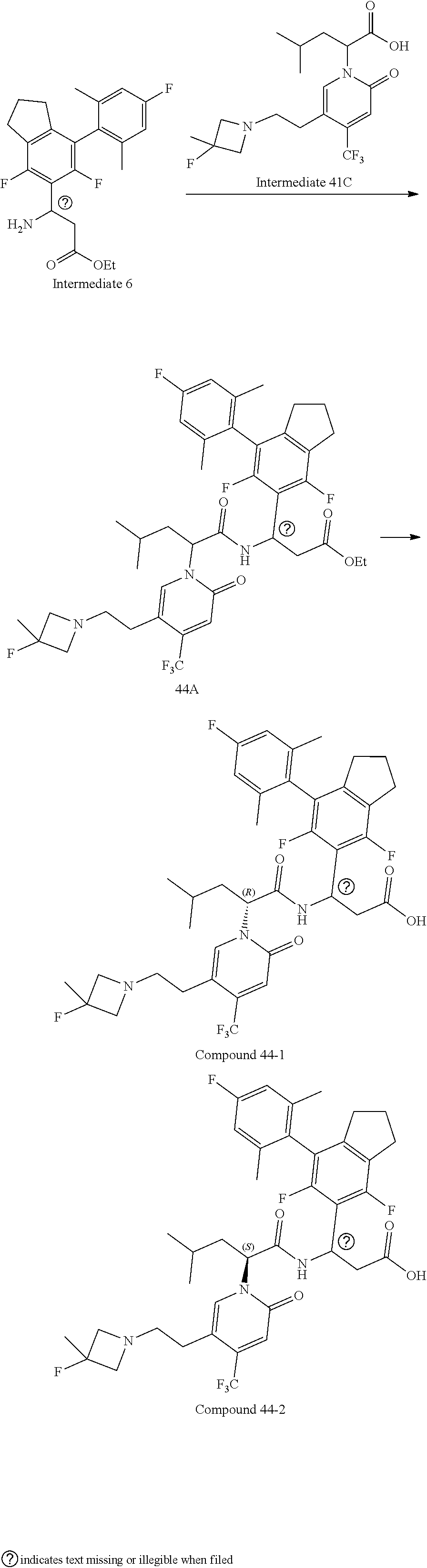

Referring to the preparation method of compound 35, crude compound 44 was obtained, which was separated and purified by prep-HPLC (method 1) to afford compound 44-1 (84 mg, Rt=4.315 min) and compound 44-2 (96 mg, Rt=4.398 min).

### Compound 44-1:

LC-Ms m/z (ESI): 738.3 [M+H]⁺

¹H NMR (400 MHz, CD3OD) δ 7.79 (s, 1H), 6.88-6.80 (m, 2H), 6.79 (s, 1H), 5.74-5.64 (m, 2H), 3.63-3.38 (m, 4H), 3.02-2.90 (m, 3H), 2.88-2.80 (m, 2H), 2.80-2.71 (m, 1H), 2.71-2.57 (m, 2H), 2.49-2.39 (m, 2H), 2.10-2.01 (m, 2H), 2.00-1.91 (m, 5H), 1.85-1.82 (m, 3H), 1.56 (d, 3H), 1.47-1.36 (m, 1H), 0.99-0.89 (m, 6H).

### Compound 44-2:

LC-Ms m/z (ESI): 738.3 [M+H]⁺

¹H NMR (400 MHz, CD₃OD) δ 7.74 (s, 1H), 6.91-6.84 (m, 3H), 5.83-5.76 (m, 1H), 5.70-5.61 (m, 1H), 4.02-3.75 (m, 4H), 3.20-3.13 (m, 2H), 3.04-2.94 (m, 2H), 2.91-2.70 (m, 3H), 2.69-2.60 (m, 1H), 2.53-2.44 (m, 2H), 2.14-2.03 (m, 2H), 1.98 (s, 6H), 1.94-1.84 (m, 1H), 1.83-1.71 (m, 1H), 1.63-1.58 (m, 3H), 1.42-1.28 (m, 1H), 0.94-0.84 (m, 6H).

### Example 45: Preparation of Compound 45

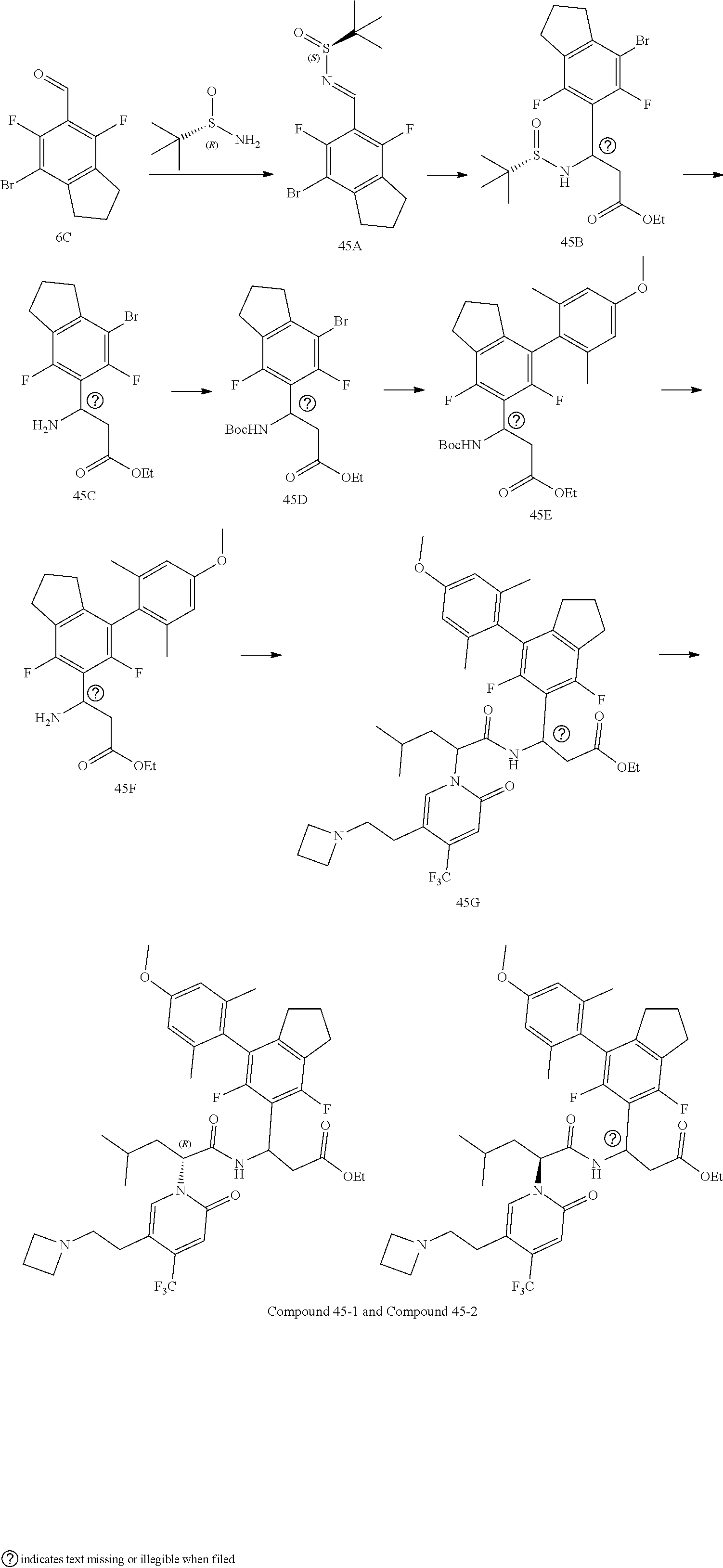

Taking (S)-tert-butylsulfinamide and 6c as starting materials, and referring to the preparation method of intermediate 6 and compound 35, crude compound 45 was obtained, which was separated and purified by prep-HPLC (method 1) to afford compound 45-1 (5 mg, retention time=3.918 min) and compound 45-2 (6 mg, retention time=4.001 min).

Compound 45-1: LC-Ms m/z (ESI): 718.3 [M+H]⁺

Compound 45-2: LC-Ms m/z (ESI): 718.3 [M+H]⁺

### Example 46: Synthesis of Compound 46

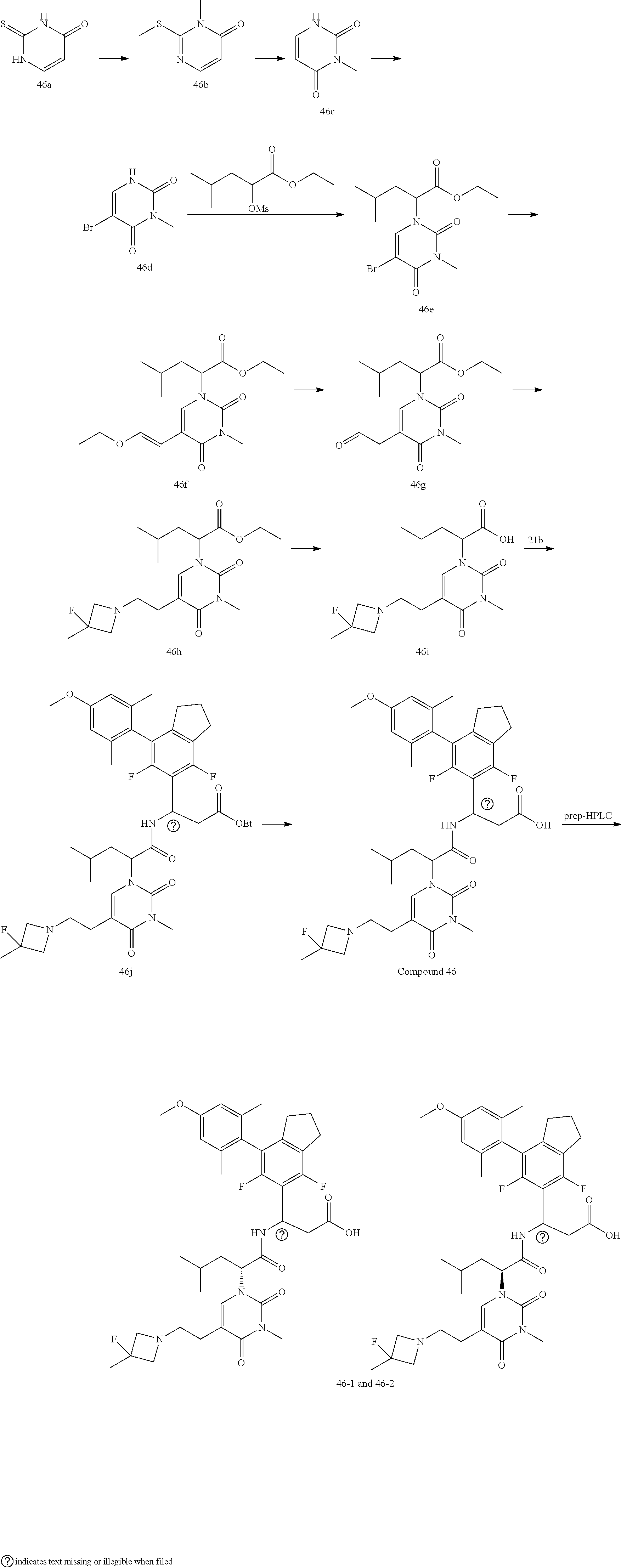

### Step 1: Synthesis of 46b

At room temperature, 2-thiouracil (10 g, 78.03 mmoL) and potassium carbonate (32 g, 234.02 mmoL) were dissolved in tetrahydrofuran solution (500 mL), iodomethane (15 mL, 234.04 mmoL) was added, and the resulting mixture was reacted for 3 hours. Water was added, and the reaction solution was extracted with ethyl acetate (50 mL×3 times). The organic phases were combined, dried over anhydrous sodium sulfate and filtered. The filtrate was concentrated under reduced pressure and the residue was purified by silica gel column chromatography to afford 46b (6.8 g, yield: 55.79%).

LC-Ms m/z (ESI): 157.2 [M+H]⁺

### Step 2: Synthesis of 46c

At room temperature, 46b (6.8 g, 43.54 mmoL) was dissolved in a mixed solution of 1,4-dioxane and water (5:1, 200 mL), potassium peroxymonosulfonate (40.15 g, 65.31 mmoL) was added, and the mixture was reacted at 100° C. for 4 hours. Water was added, and the reaction solution was extracted with ethyl acetate (50 mL×3 times). The organic phases were combined, dried over anhydrous sodium sulfate and filtered. The filtrate was concentrated under reduced pressure and the residue was purified by silica gel column chromatography to afford compound 46c (4.9 g, yield: 89.24%).

LC-Ms m/z (ESI): 127.2 [M+H]⁺

### Step 3: Synthesis of 46d

At room temperature, 46c (4.9 g, 38.87 mmoL) was dissolved in 50 mL acetonitrile, N-bromosuccinimide (6.92 g, 38.87 mmoL) and glacial acetic acid (0.22 mL, 3.85 mmoL) were separately added, and the resulting mixture was reacted for 4 hours. Sodium bicarbonate aqueous solution was added to quench the reaction, and the reaction solution was extracted with ethyl acetate (50 milliliter×3 times). The organic phases were combined, dried over anhydrous sodium sulfate and filtered. The filtrate was concentrated under reduced pressure and the residue was purified by silica gel column chromatography to afford compound 46d (5.5 g, yield: 69.02%).

LC-Ms m/z (ESI): 206.1 [M+H]⁺

### Step 4: Synthesis of 46e

At room temperature, 46d (5.5 g, 26.83 mmoL), and intermediate ethyl 2-(methanesulfonyloxy)-4-methylpentanoate (7.67 g, 32.20 mmoL) were dissolved in acetonitrile (80 mL), and potassium carbonate (11.12 g, 80.53 mmoL) was added. The reaction system was then warmed to 80° C. and allowed to react overnight. The reaction liquid was filtered over celite, and extracted with ethyl acetate (30 mL×3 times). The organic phases were combined, dried over anhydrous sodium sulfate and filtered. The filtrate was concentrated under reduced pressure and the residue was purified by silica gel column chromatography to afford compound 46e (4.7 g, yield: 50.45%).

LC-Ms m/z (ESI): 348.2 [M+H]⁺

### Step 5: Synthesis of 46f

46e (4.7 g, 13.54 mmoL), and (E)-1-ethoxyvinyl-2-boronic acid pinacol ester (3.22 g, 16.25 mmoL) were dissolved in 1,4-dioxane and water (v/v=10:1, 110 mL). Potassium carbonate (5.61 g, 40.62 mmoL) and tetrakistriphenylphosphine palladium (1.56 g, 1.35 mmoL) were added, and the reaction system was subjected to nitrogen replacement 3 times and reacted at 70° C. for 24 hours. Water was added to quench the reaction, and the reaction solution was extracted with ethyl acetate (30 mL×3 times). The organic phases were combined, washed with saturated sodium chloride aqueous solution (50 mL), dried over anhydrous sodium sulfate and filtered. The filtrate was concentrated under reduced pressure and the residue was purified by silica gel column chromatography to afford 46f (1.8 g, yield: 39.28%).

LC-Ms m/z (ESI): 339.4 [M+H]⁺

### Step 6: Synthesis of Compound 46g

46f (1.8 g, 5.32 mmoL) was dissolved in dichloromethane (20 mL), trifluoroacetic acid (10 mL, 134.19 mmoL) was added slowly and the reaction was allowed to proceed overnight at room temperature. The reaction solution was concentrated under reduced pressure, the pH was adjusted to neutral by adding aqueous sodium bicarbonate solution, and the reaction solution was extracted with ethyl acetate (20 mL×2). The organic phases were combined, dried over anhydrous sodium sulfate, filtered and concentrated under reduced pressure to obtain crude 46g, which was directly used in the next reaction without purification.

### Steps 7 to 10: Synthesis of Compound 46

Taking 46g as starting material and referring to the synthetic route and preparation method of compound 3, crude compound 46 was obtained, which was separated and purified by prep-HPLC (preparation method 1) to afford compound 46-1 (20 mg, retention time=4.122 min) and compound 46-2 (18 mg, retention time=4.250 min).

### Compound 46-1:

LC-Ms m/z (ESI): 713.3 [M+H]⁺

¹H NMR (400 MHz, CD₃OD) δ 7.70 (s, 1H), 6.68 (s, 2H), 5.73-5.65 (m, 1H), 5.45-5.37 (m, 1H), 4.47-4.26 (m, 4H), 3.79 (s, 3H), 3.56-3.38 (m, 2H), 3.25 (s, 3H), 3.12-3.02 (m, 1H), 2.99-2.91 (m, 2H), 2.89-2.81 (m, 1H), 2.80-2.71 (m, 1H), 2.66-2.56 (m, 1H), 2.52-2.39 (m, 2H), 2.10-2.01 (m, 2H), 2.00-1.90 (m, 4H), 1.89-1.75 (m, 4H), 1.71-1.61 (m, 3H), 1.54-1.43 (m, 1H), 1.00-0.91 (m, 6H).

### Compound 46-2:

LC-Ms m/z (ESI): 713.3 [M+H]⁺

¹H NMR (400 MHz, CD₃OD) δ 7.71 (s, 1H), 6.70 (s, 2H), 5.74-5.64 (m, 1H), 5.48-5.40 (m, 1H), 4.52-4.26 (m, 4H), 3.79 (s, 3H), 3.58-3.38 (m, 2H), 3.32 (s, 3H), 3.09-3.01 (m, 1H), 3.00-2.93 (m, 2H), 2.91-2.72 (m, 2H), 2.66-2.56 (m, 1H), 2.53-2.44 (m, 2H), 2.12-2.01 (m, 2H), 1.94 (s, 6H), 1.83-1.73 (m, 1H), 1.71-1.60 (m, 4H), 1.45-1.32 (m, 1H), 0.95-0.85 (t, 6H).

### Example 47 Preparation of Compound 47

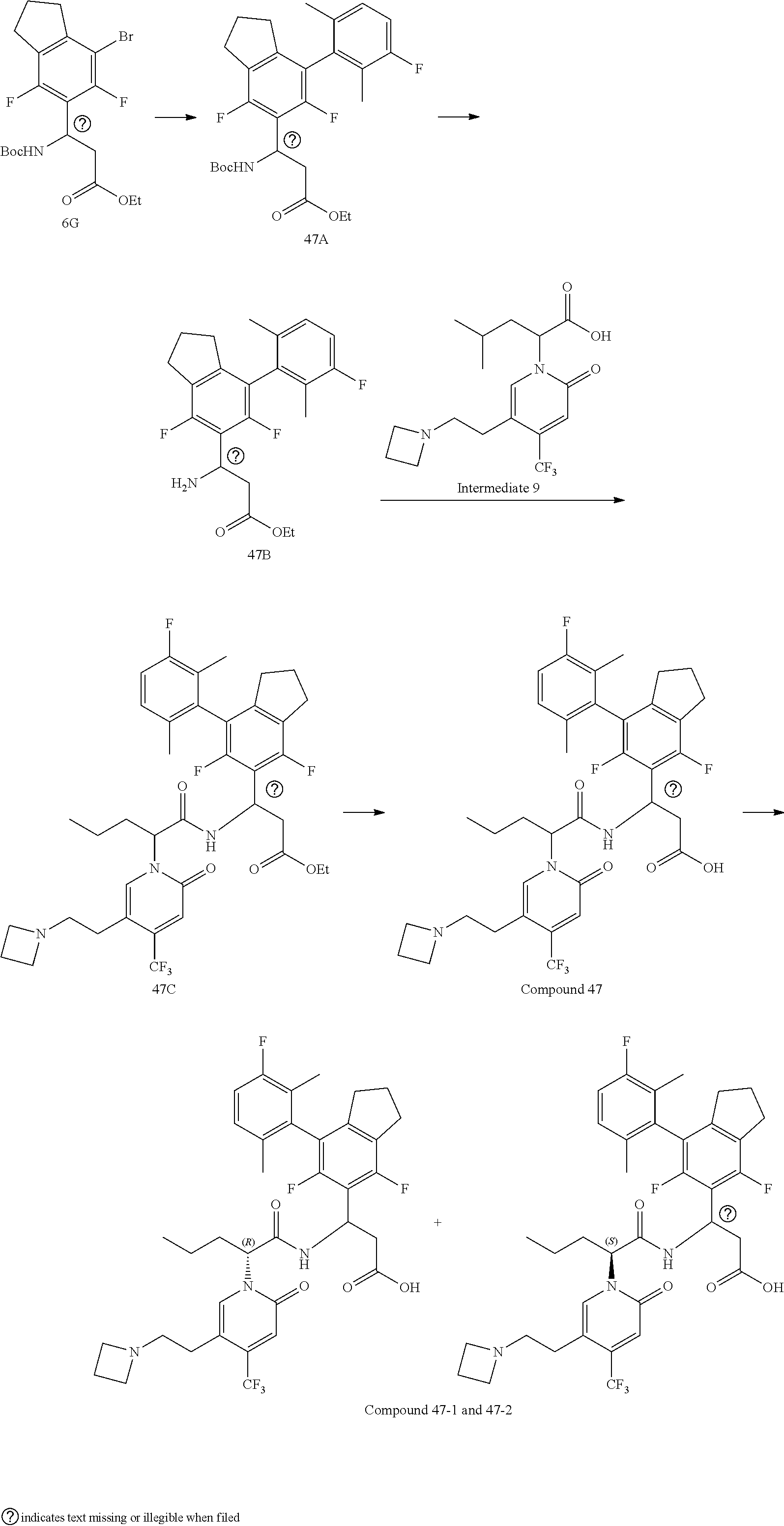

Referring to the synthetic route and preparation method of compound 35, crude compound 47 was obtained, which was separated and purified by prep-HPLC (preparation method 2) to afford compound 47-1 (40 mg, retention time=4.179 min) and compound 47-2 (45 mg, retention time=4.271 min).

### Trifluoroacetate Salt of Compound 47-1:

LC-Ms m/z (ESI): 706.3 [M+H]⁺

¹H NMR (400 MHz, CD₃OD) δ 9.26 (d, 1H), 7.86 (s, 1H), 7.15-7.05 (m, 1H), 7.02-6.93 (m, 1H), 6.82 (s, 1H), 5.85-5.65 (m, 2H), 4.42-4.03 (m, 4H), 3.44-3.33 (m, 2H), 3.15-3.05 (m, 1H), 3.00-2.78 (m, 5H), 2.67-2.53 (m, 1H), 2.51-2.36 (m, 3H), 2.13-1.99 (m, 3H), 1.97-1.76 (m, 7H), 1.47-1.35 (m, 1H), 1.00-0.90 (m, 6H).

### Trifluoroacetate Salt of Compound 47-2:

LC-Ms m/z (ESI): 706.3 [M+H]⁺

¹H NMR (400 MHz, CD₃OD) δ 9.38 (d, 1H), 7.88 (s, 1H), 7.17-7.10 (m, 1H), 7.04-6.96 (m, 1H), 6.88 (s, 1H), 5.83-5.66 (m, 2H), 4.42-4.05 (m, 4H), 3.50-3.33 (m, 2H), 3.11-2.95 (m, 3H), 2.95-2.80 (m, 3H), 2.71-2.56 (m, 1H), 2.53-2.39 (m, 3H), 2.15-2.04 (m, 2H), 1.95 (s, 3H), 1.90 (d, 3H), 1.87-1.71 (m, 2H), 1.37-1.23 (m, 1H), 0.98-0.83 (m, 6H).

### Example 48: Preparation of Compound 48

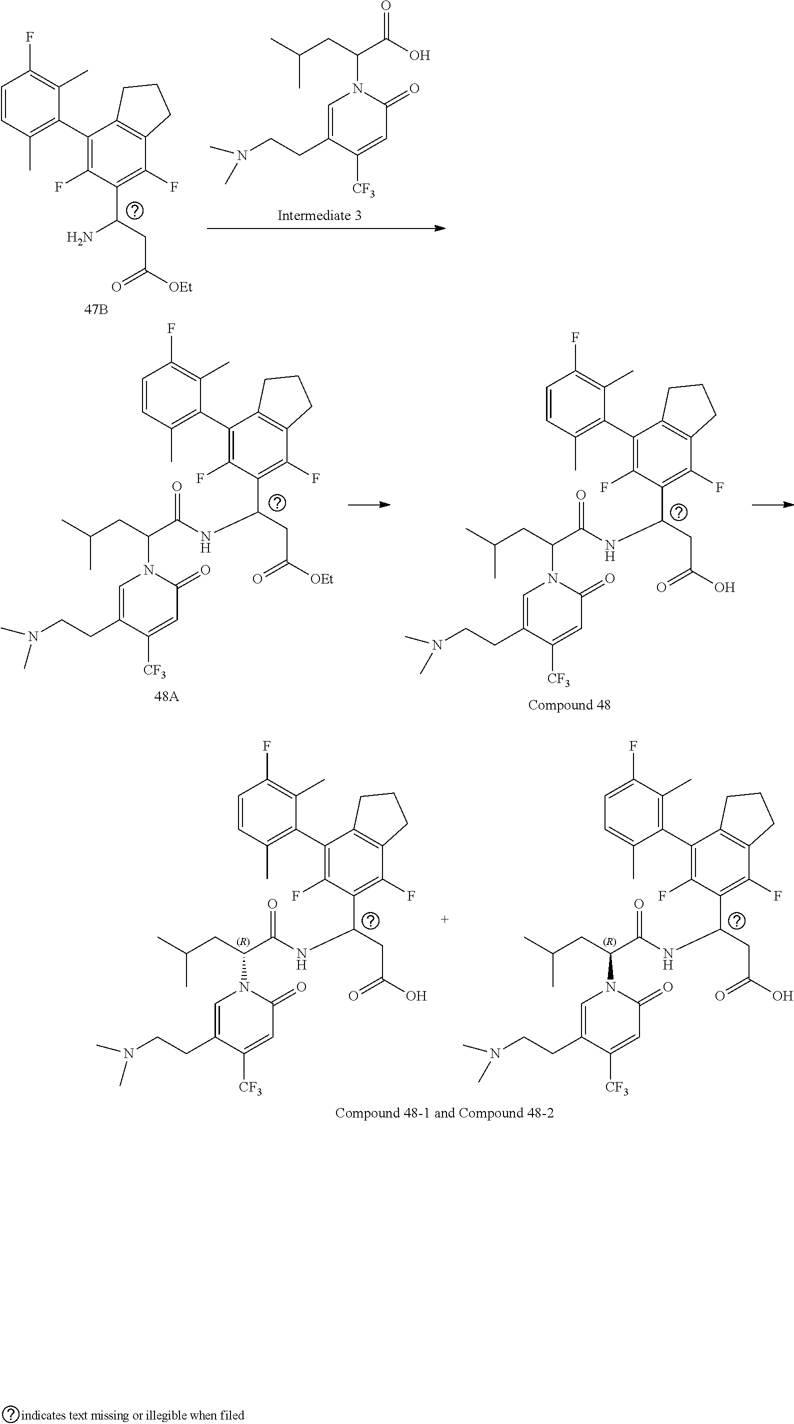

Referring to the synthetic route and preparation method of compound 35, crude compound 48 was obtained, which was separated and purified by prep-HPLC (preparation method 2) to afford compound 48-1 (54 mg, retention time=4.179 min) and compound 48-2 (62 mg, retention time=4.271 min).

### Trifluoroacetate Salt of Compound 48-1:

LC-Ms m/z (ESI): 694.3 [M+H]⁺

¹H NMR (400 MHz, CD₃OD) δ 9.23 (d, 1H), 7.90 (s, 1H), 7.13-7.04 (m, 1H), 7.01-6.91 (m, 1H), 6.82 (s, 1H), 5.82-5.74 (m, 1H), 5.73-5.64 (m, 1H), 3.36-3.22 (m, 2H), 3.15-2.83 (m, 12H), 2.52-2.36 (m, 2H), 2.13-1.98 (m, 3H), 1.97-1.74 (m, 7H), 1.48-1.35 (m, 1H), 1.01-0.94 (m, 6H).

### Trifluoroacetate Salt of Compound 48-2:

LC-Ms m/z (ESI): 694.3 [M+H]⁺

¹H NMR (400 MHz, CD₃OD) δ 9.35 (d, 1H), 7.92 (s, 1H), 7.16-7.08 (m, 1H), 7.04-6.94 (m, 1H), 6.88 (s, 1H), 5.82-5.64 (m, 2H), 3.39-3.24 (m, 2H), 3.10-2.84 (m, 12H), 2.55-2.45 (m, 2H), 2.16-2.03 (m, 2H), 1.95 (s, 3H), 1.90 (d, 3H), 1.87-1.71 (m, 2H), 1.38-1.23 (m, 1H), 0.98-0.81 (m, 6H).

### Example 49: Preparation of Compound 49

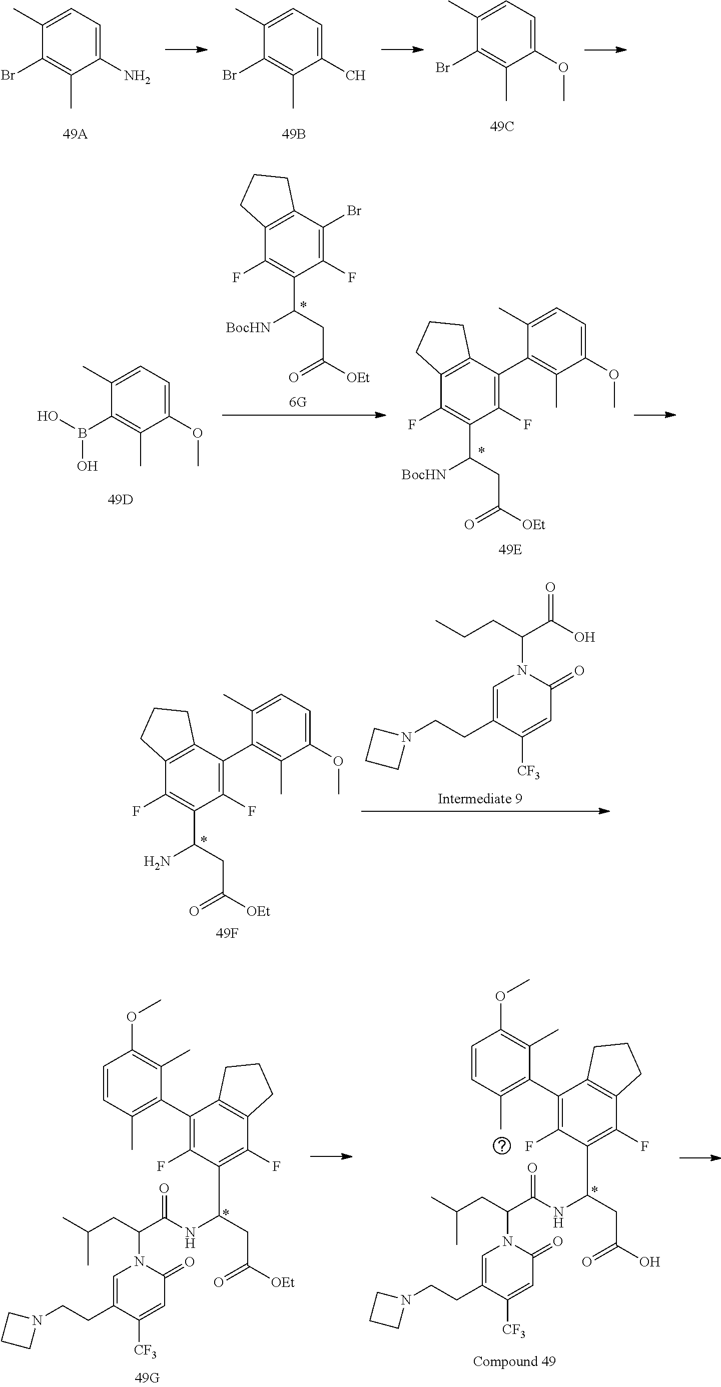

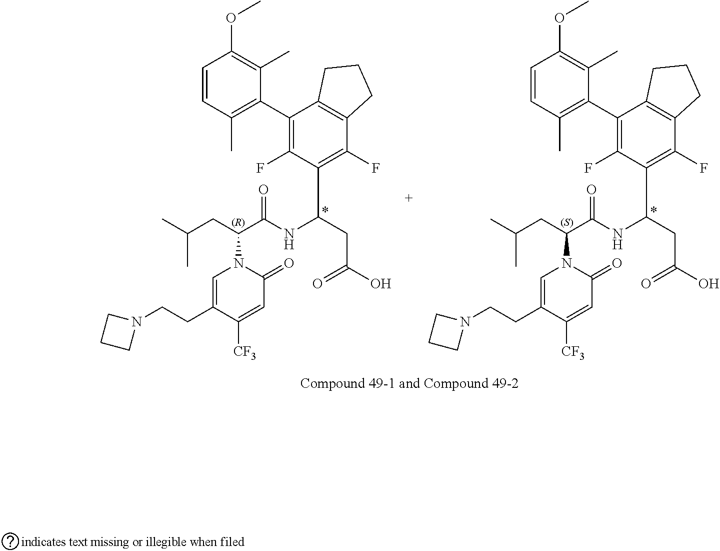

### Step 1: Synthesis of Compound 49B

Substrate 49A (5.00 g, 24.99 mmo) was dissolved in 10% aqueous sulfuric acid (50 mL) under ice bath conditions, then sodium nitrite (1.72 g, 24.99 mmol) was dissolved in water (10 mL) and then added dropwise to the system. After the addition was complete, the reaction was continued under ice bath conditions for 1 hour; 50% aqueous sulfuric acid solution (50 mL) was added dropwise to the system, and after the addition was complete, the reaction system was warmed to 100° C. and reacted for 1 hour. The reaction system was cooled to room temperature, and the reaction liquid was slowly added to a stirred ice water (300 mL). The stirring was continued for 30 minutes, and the aqueous phase was extracted with ethyl acetate (50 mL×3). The organic phases were combined, dried over anhydrous sodium sulfate, filtered and concentrated to afford a crude product, which was purified by silica gel column chromatography to afford 49B (2.89 g, yield: 57.52%).

### Step 2: Synthesis of Compound 49C

Substrate 49B (2.89 g, 14.37 mmo) was dissolved in acetonitrile (30 mL), and potassium carbonate (3.97 g, 28.72 mmol) and iodomethane (3.06 g, 21.55 mmol) were added. After the addition was complete, the reaction system was warmed to 60° C. and reacted for 18 hours under nitrogen atmosphere. The reaction was cooled to room temperature and filtered, and the filter cake was washed with ethyl acetate (5 mL×2). The filtrate was collected and concentrated to obtain a crude product, which was purified by silica gel column chromatography to afford 49C (2.65 g, yield: 85.74%).

### Step 3: Synthesis of Compound 49D

Substrate 49C (2.65 g, 12.32 mmol) was dissolved in ultra-dry tetrahydrofuran (40 mL), and pre-cooled under nitrogen atmosphere at −78° C. for 10 minutes. N-butyllithium (0.89 g, 13.91 mmol) was slowly added dropwise, and after the addition was complete, stirring was continued at −78° C. for 1 hour. Trimethyl borate (1.71 g, 16.47 mmol) was added dropwise to the system, and after the addition was complete, the reaction system was naturally warmed to room temperature and reacted for 18 hours under nitrogen atmosphere. The reaction was poured into 10% aqueous hydrochloric acid solution (50 mL) under ice bath conditions, stirred for 30 minutes under ice bath conditions, and the reaction solution was extracted with ethyl acetate (30 mL 3). The organic phases were combined, dried over anhydrous sodium sulfate, filtered and concentrated to afford a crude product, which was purified by silica gel column chromatography to afford 49D (0.84 g, yield: 37.88%).

Referring to the synthetic route and preparation method of compound 35, crude compound 49 was obtained, which was separated and purified by prep-HPLC (preparation method 1) to afford compound 49-1 (32 mg, retention time=4.218 min) and compound 49-2 (35 mg, retention time=4.303 min).

### Compound 49-1:

LC-Ms m/z (ESI): 718.3 [M+H]⁺

¹H NMR (400 MHz, CD₃OD) δ 7.76 (s, 1H), 7.06 (d, 1H), 6.88 (s, 1H), 6.85 (d, 1H), 5.75-5.68 (m, 1H), 5.67-5.59 (m, 1H), 4.05-4.39 (m, 4H), 3.82 (s, 3H), 3.37-3.31 (m, 2H), 2.96 (t, 2H), 2.92-2.82 (m, 3H), 2.69-2.60 (m, 1H), 2.49-2.36 (m, 4H), 2.12-2.01 (m, 2H), 2.00-1.92 (m, 2H), 1.89-1.76 (m, 6H), 1.47-1.34 (m, 1H), 0.97-0.87 (m, 6H).

### Compound 49-2:

LC-Ms m/z (ESI): 718.3 [M+H]⁺

¹H NMR (400 MHz, CD₃OD) δ 7.66 (s, 1H), 7.08 (d, 1H), 6.92 (s, 1H), 6.86 (d, 1H), 5.92-5.86 (m, 1H), 5.60 (t, 1H), 4.11 (t, 4H), 3.83 (s, 3H), 3.46-3.32 (m, 2H), 3.03-2.87 (m, 3H), 2.86-2.71 (m, 2H), 2.53-2.39 (m, 5H), 2.13-2.02 (m, 2H), 1.99-1.92 (m, 1H), 1.91-1.81 (m, 6H), 1.78-1.67 (m, 1H), 1.43-1.32 (m, 1H), 0.93-0.84 (m, 6H).

### Example 50: Preparation of Compound 50

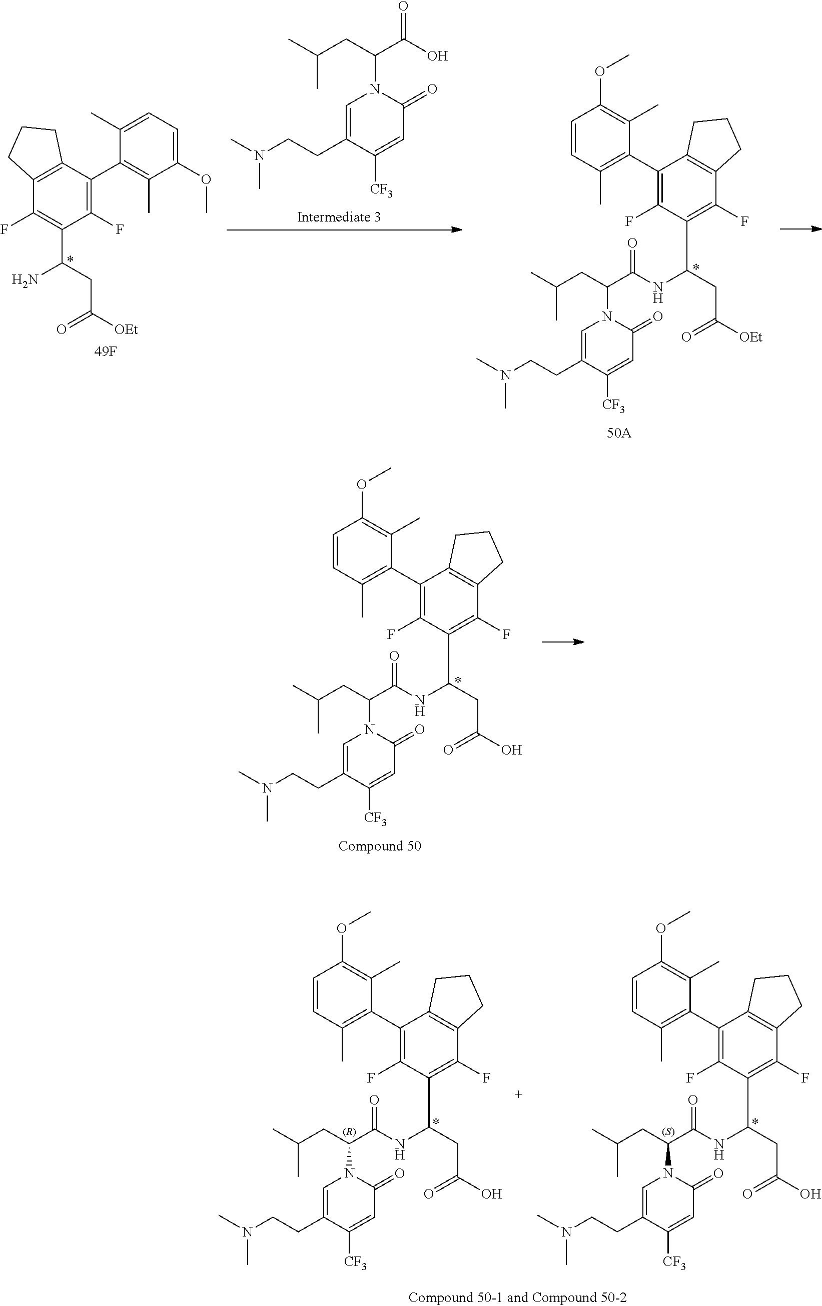

Referring to the synthetic route and preparation method of compound 35, crude compound 50 was obtained, which was separated and purified by prep-HPLC (preparation method 1) to afford compound 50-1 (35 mg, retention time=4.184 min) and compound 50-2 (37 mg, retention time=4.260 min).

### Compound 50-1:

LC-Ms m/z (ESI): 706.4 [M+H]⁺

1H NMR (400 MHz, CD₃OD) δ 7.85 (s, 1H), 7.08-7.02 (m, 1H), 6.87-6.81 (m, 2H), 5.72-5.63 (m, 2H), 3.82 (s, 3H), 3.14-3.00 (m, 2H), 2.99-2.87 (m, 5H), 2.71 (m, 6H), 2.70-2.63 (m, 1H), 2.46-2.39 (m, 2H), 2.09-2.00 (m, 2H), 1.99-1.91 (m, 2H), 1.89-1.72 (m, 6H), 1.47-1.35 (m, 1H), 0.97-0.88 (m, 6H).

### Compound 50-2:

LC-Ms m/z (ESI): 706.4 [M+H]⁺

1H NMR (400 MHz, CD₃OD) δ 7.79 (s, 1H), 7.08 (d, 1H), 6.90 (s, 1H), 6.86 (d, 1H), 5.87-5.78 (m, 1H), 5.61 (t, 1H), 3.83 (s, 3H), 3.25-3.11 (m, 2H), 3.03-2.92 (m, 4H), 2.83-2.72 (m, 7H), 2.58-2.50 (m, 1H), 2.46 (t, 2H), 2.14-2.02 (m, 2H), 1.98-1.91 (m, 1H), 1.90-1.81 (m, 6H), 1.79-1.69 (m, 1H), 1.42-1.31 (m, 1H), 0.94-0.81 (m, 6H).

### Biological Test:

### 1. ELISA Detection Assay for Integrin α4β7:

MAdCAM was formulated with TBS buffer to a final concentration of 2.5 ug/ml, 50 ul of the solution was transferred to a 96-well plate, and the plate was coated with same at 4° C. overnight. The plate was washed with TBS buffer three times, then 150 ul of a blocking solution (TBS buffer containing 1% BSA) was added, and the plate was blocked at 37° C. for 1 h. The plate was washed with TBS buffer three times. 1 μg/ml α4β7 integrin was formulated using TBS buffer containing 0.1% BSA. 50 ul of integrin was transferred to a 96-well plate, then 1 ul of the compound at different concentrations or DMSO was added, and the plate was incubated at normal temperature for 2 h. 1 μg/ml biotinylated anti-07 antibody was formulated using TBS buffer containing 0.1% BSA. The plate was washed with TBS buffer three times, 50 ul of the antibody was added, and the plate was incubated at normal temperature for 1 h. The plate was washed with TBS buffer three times, 50 ul of streptavidin-HRP was added, and the plate was incubated at normal temperature for 20 min. The plate was washed with TBS buffer three times, 50 ul of a TMB substrate was added, and the plate was incubated at normal temperature for 5-30 min. Finally, 25 ul of a termination buffer (phosphate solution at a high concentration) was added. The OD value of the plate was read at 450 nm with a microplate reader. The IC₅₀ value was calculated using GraphPad Prism 6 software.

Conclusion: The compounds of the present disclosure, for example, the compounds of the Examples have good inhibitory activity against integrin α4β7.

### 2. Integrin α4β7 Cell Adhesion Assay

MAdCAM-1 was formulated with TBS buffer to a final concentration of 2 g/ml, 50 μl of the solution was transferred to a 96-well plate, and the plate was coated with same at 4° C. overnight. The plate was washed with TBS buffer three times, then 150 μl of a blocking solution (TBS buffer containing 1% BSA) was added, and the plate was blocked at 37° C. for 1 h. RPMI8866 cells were collected, washed twice with DPBS buffer, and then resuspended to 4×10⁵ cells/ml with TBS buffer. 50 μl of the cell suspension was transferred to a 96-well plate so that the cell density reached 2×10⁵ cells/well. Then 1 μl of the compound at different concentrations or DMSO was added and the plate was incubated at 37° C. for 1 h or 2 h. The plate was washed with TBS buffer to remove non-adherent cells. 50 μl of a substrate (4-nitrophenyl-N-acetyl-o-D-glucosaminide) was added and the plate was incubated at 37° C. for 2 h. Finally, 90 μl of a termination solution (50 mM glycine and 5 mM EDTA, pH 10.4) was added. The OD value of the plate was read at 405 nm with a microplate reader. The IC₅₀ value was calculated using GraphPad Prism 6 software. The results are shown in Table 1.

### 3. Integrin α4β1-Mediated Cell Adhesion Assay

VCAM was formulated into a final concentration of 0.5 g/ml with a coating buffer, 50 μl of the solution was transferred to a 96-well plate, and the plate was coated with same at 4° C. overnight. The plate was washed with coating buffer three times, then 150 μl of a blocking solution (coating buffer containing 1% BSA) was added, and the plate was blocked at 37° C. for 1 h. Jurkat cells were collected, washed twice using DPBS buffer, and then resuspended to 4×10⁶ cells/ml with an action buffer. 50 μl of the cell suspension was transferred to a 96-well plate so that the cell density reached 2×10⁵ cells/well. Then 1 μl of the test compound at different concentration or DMSO was added and the plate was incubated at 37° C. for 1 h. The plate was washed with coating buffer to remove non-adherent cells. 50 μl of a substrate (4-nitrophenyl-N-acetyl-β-D-glucosaminide) was added and the plate was incubated at 37° C. for 2 h. Finally, 90 μl of a termination solution (50 mM glycine and 5 mM EDTA, pH 10.4) was added. The OD value of the plate was read at 405 nm.

Coating buffer: DMEM+20 mM HEPES+0.4 mM MnCl₂

Action buffer: DMEM+20 mM HEPES+0.1% BSA+0.4 mM MnCl₂

DMEM: dulbecco's modified eagle medium;

HEPES: 2-[4-(2-hydroxyethyl)piperazin-1-yl]ethanesulfonic acid

TABLE 1
Results of effects of compounds on α4β7- and α4β1-mediated
cell adhesion

| | RPMI-8866 | Jurkat | |
|---|---|---|---|
| | (α4β7)/ | (α4β1)/ | |
| | MadCAM-1 | VCAM-1 | α4β1/ |
| Compound No. | IC₅₀ (nM) | IC50 (nM) | α4β7 |
| Compound 1-2 | A | >100 | >50 |
| Trifluoroacetate salt of | A | >300 | 106 |
| compound 2-2 | | | |
| Compound 3-2 | A | / | / |
| Trifluoroacetate salt of | A | >500 | 648 |
| compound 4-2 | | | |
| Compound 6-2 | A | / | / |
| Compound 7-2 | A | >500 | 182 |
| Trifluoroacetate salt of | <0.5 | >30 | 269 |
| compound 9-2 | | | |
| Trifluoroacetate salt of | <0.5 | >100 | 1423 |
| compound 10-2 | | | |
| Trifluoroacetate salt of | <0.5 | >100 | 269 |
| compound 11-2 | | | |
| Trifluoroacetate salt of | <0.5 | >100 | 354 |
| compound 12-2 | | | |
| Trifluoroacetate salt of | <0.5 | >100 | 454 |
| compound 14-2 | | | |
| Trifluoroacetate salt of | <1 | >100 | 272 |
| compound 15-2 | | | |
| Trifluoroacetate salt of | <1 | >200 | 308 |
| compound 16-2 | | | |
| Trifluoroacetate salt of | <1 | >100 | 165 |
| compound 17-2 | | | |
| Trifluoroacetate salt of | A | >500 | 258 |
| compound 18-2 | | | |
| Trifluoroacetate salt of | A | >500 | 323 |
| compound 19-2 | | | |
| Trifluoroacetate salt of | A | >500 | 648 |
| compound 20-2 | | | |
| Trifluoroacetate salt of | <1 | >500 | 1586 |
| compound 21-2 | | | |
| Trifluoroacetate salt of | <0.5 | >100 | 307 |
| compound 22-2 | | | |
| Trifluoroacetate salt of | A | >500 | 310 |
| compound 23-2 | | | |
| Compound 24-2 | A | / | / |
| Compound 25-2 | <0.5 | >50 | 194 |
| Trifluoroacetate salt of | <1 | >100 | 177 |
| compound 26-2 | | | |
| Trifluoroacetate salt of | A | >100 | 133 |
| compound 27-2 | | | |
| Trifluoroacetate salt of | A | >100 | 130 |
| compound 28-2 | | | |
| Trifluoroacetate salt of | <1 | >200 | 292 |
| compound 29-2 | | | |
| Trifluoroacetate salt of | <1 | >100 | 220 |
| compound 32-2 | | | |
| Compound 33-2 | <0.5 | >100 | 643 |
| Compound 35-2 | <0.5 | >300 | 3651 |
| Compound 36-2 | 0.5 | >100 | 240 |
| Compound 38-2 | <0.5 | >100 | 862 |
| Compound 39-2 | A | >500 | 213 |
| Compound 21-A | A | >100 | 119 |
| Compound 40-2 | <0.5 | >300 | 1143 |
| Compound 43-2 | <1 | 90.7 | >100 |
| Compound 46-2 | A | >4000 | >1200 |
| Trifluoroacetate salt of | <1 | | |
| compound 48-2 | | | |
| Compound 49-2 | A | | |
| Compound 50-2 | A | | |
| Control compound A | <0.5 | <5 | 50.4 |

A <10 nM; control compound A is

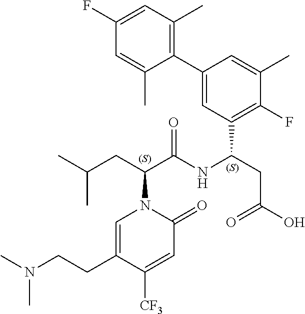

Conclusion: The compounds of the present disclosure, such as the compounds of the Examples, have a good inhibitory effect on integrin α4β7-mediated cell adhesion and are more selective than the control compound A.

### 4. CYP450 Enzyme Inhibition Test

The purpose of this study was to evaluate the effect of the test compound on the activity of five isoenzymes (CYP1A2, CYP2C9, CYP2D6 and CYP3A4) of human liver microsomal cytochrome P450 (CYP) by using an in vitro testing system. The specific probe substrates of CYP450 isoenzymes were incubated with human liver microsomes and test compounds of different concentrations, and reduced nicotinamide adenine dinucleotide phosphate (NADPH) was added to initiate the reaction. After the completion of the reaction, the sample was treated and liquid chromatography-tandem mass spectrometry (LC-MS/MS) was used to quantitatively detect metabolites produced by specific substrates, changes in CYP enzyme activity were determined, and IC50 value was calculated to evaluate the inhibitory potential of the test compound on each CYP enzyme subtype CYP1A2, CYP2C9, CYP2D6, or CYP3A4-M (midazolam was used as the substrate).

Conclusion: The compounds of the present disclosure, such as the compounds of the Examples, have no significant inhibitory effect on various subtypes of CYP enzyme.

### 5. Pharmacokinetic Test in Mice

Experimental animals: male BALB/c mice, 20-25 g, 6 mice/compound. purchased from Chengdu Ddossy Experimental Animals Co., Ltd.

Experimental design: on the day of the test, 6 BALB/c mice were randomly grouped according to their body weights. The animals were fasted with water available for 12 to 14 h one day before the administration and were fed 4 h after the administration.

TABLE 2
Administration information

| | Number | Administration information | | | | | |
|---|---|---|---|---|---|---|---|
| | | | Administration | Administration | Administration | | |
| | | Test | dosage | concentration | volume | Collected | Mode of |
| Group | Male | compound | (mg/kg) | (mg/mL) | (mL/kg) | samples | administration |
| G1 | 3 | Compound | 1 | 0.2 | 5 | Plasma | Intravenous |
| | | | | | | | administration |
| G2 | 3 | | 10 | 1 | 10 | Plasma | Intragastric |
| | | | | | | | administration |

Note: solvent for intravenous administration: 5% DMA + 5% Solutol + 90% Saline;
solvent for intragastric administration: 5% DMSO+95% (20% SBE-ß-CD in saline)
(DMA: dimethylacetamide; Solutol: polyethylene glycol-15-hydroxystearate; Saline:
physiological saline; DMSO: dimethyl sulfoxide; SBE-ß-CD: sulfobutyl-ß-cyclodextrin)
Before and after the administration, 0.06 mL of blood was taken from the orbit of the
animals under isoflurane anesthesia and placed in an EDTAK2 centrifuge tube. The
blood was centrifuged at 5000 rpm and 4°° C. for 10 min to collect plasma. The blood
collection time points for the intravenous group and intragastric group were both 0 min, 5
min, 15 min, 30 min, 1 h, 2 h, 4 h, 6 h, 8 h, and 24 h. Before analysis and detection, all
samples were stored at -80°° C., and the samples were quantitatively analyzed by LC-
MS/MS.

TABLE 3

| | IV: 1 mg/kg | PO: 10 mg/kg | |
|---|---|---|---|
| | CL | AUC | |
| Compound No. | (mL · kg⁻¹ · min⁻¹) | (h · ng · mL⁻¹) | F |
| Compound 21-2 | 22.6 | 1380 | |
| Compound 35-2 | 13.3 | 1259 | / |
| Compound 36-2 | 14.0 | 5501 | 45.9% |
| Control | 56.0 | 830 | 28.2% |
| compound A | | | |

Conclusion: The compounds of the present disclosure, such as the compounds of the Examples, have good oral absorption performance and lower clearance rate in mice.

### 6. Pharmacokinetic Test in Rats

Experimental animals: Male SD rats, about 220 g, 6-8 weeks old, 6 rats/compound, purchased from Chengdu Ddossy Experimental Animals Co., Ltd.

Experimental design: on the day of the test, 6 SD rats/compound were randomly grouped according to their body weights. The animals were fasted with water available for 12 to 14 h one day before the administration and were fed 4 h after the administration.

TABLE 4
Administration information

| | Number | Administration information | | | | | |
|---|---|---|---|---|---|---|---|
| | | | Administration | Administration | Administration | | Mode |
| | | Test | dosage | concentration | volume | Collected | of |
| Group | Male | compound | (mg/kg) | (mg/mL) | (mL/kg) | samples | administration |
| G1 | 3 | Compound | 1 | 0.2 | 5 | Plasma | Intravenous |
| | | of the | | | | | administration |
| G2 | 3 | present | 7.5 | 0.75 | 10 | Plasma | Intragastric |
| | | disclosure | | | | | administration |
| G3 | 3 | Control | 2.5 | 0.5 | 5 | Plasma | Intravenous |
| | | compound | | | | | administration |
| G4 | 3 | A | 10 | 1 | 10 | Plasma | Intragastric |
| | | | | | | | administration |

Note: solvent for intravenous administration: 5% DMA + 5% Solutol + 90% Saline;
vehicle for intragastric administration: 5% DMSO+95% (20% SBE-ß-CD in saline)
(DMA: dimethylacetamide; Solutol: polyethylene glycol-15-hydroxystearate; Saline:
physiological saline; DMSO: dimethyl sulfoxide; SBE-ß-CD: sulfobutyl-ß-cyclodextrin)
Before and after administration, 0.10 mL of blood was taken from the orbit under
isoflurane anesthesia and placed in an EDTAK2 centrifuge tube for centrifugation at
5000 rpm at 4° C. for 10 min, followed by plasma collection. The blood collection time
points for the intravenous administration group and intragastric administration group
were: 0, 5 min, 15 min, 30 min, 1 h, 2 h, 4 h, 6 h, 8 h and 24 h. Before analysis and
detection, all samples were stored at -80°° C., and the samples were quantitatively analyzed
by LC-MS/MS.

TABLE 5

| | IV: 1 mg/kg CL | PO: 7.5 mg/kg | |
|---|---|---|---|
| Compound No. | (mL · kg⁻¹ · min⁻¹) | AUC (h · ng · mL⁻¹) | F |
| Compound 21-2 | 17.2 | 2264 | 31.2% |
| Compound 32-2 | 20.2 | / | / |
| Compound 35-2 | 8.76 | 3084 | / |
| Compound 36-2 | 8.84 | 3172 | / |
| Control compound | 45.8 | 900 | 23.9% |
| A* | | | |

*Control compound A iv 2.5 mg/kg; PO: 10 mg/kg

Conclusion: The compounds of the present disclosure, such as the compounds of the Examples, have good oral absorption performance and/or lower clearance rate in rats.

### 7. Pharmacokinetic Test in Beagle Dogs

Experimental animals: male beagle dogs, about 8-11 kg, 5-6 beagle dogs/compound, purchased from Beijing Marshall Biotechnology Co., Ltd.

Experimental method: on the day of the test, 5-6 beagle dogs/compound were randomly grouped according to their body weights. The animals were fasted with water available for 12 to 14 h one day before the administration and were fed 4 h after the administration.

TABLE 6
Administration information

| | Number | Administration information | | | | | |
|---|---|---|---|---|---|---|---|
| | | | Administration | Administration | Administration | | |
| | | Test | dosage | concentration | volume | Collected | Mode of |
| Group | Male | compound | (mg/kg) | (mg/mL) | (mL/kg) | samples | administration |
| G1 | 2 or 3 | Test | 1 | 0.5 | 2 | Plasma | Intravenous |
| | | compound | | | | | administration |
| G2 | 3 | | 5 | 1 | 5 | Plasma | Intragastric |
| | | | | | | | administration |

Note: solvent for intravenous administration: 5% DMA + 5% Solutol + 90% Saline;
vehicle for intragastric administration: 5% DMSO+95% (20% SBE-B-CD in saline)
(DMA: dimethylacetamide; Solutol: polyethylene glycol-15-hydroxystearate; Saline:
physiological saline; DMSO: dimethyl sulfoxide; SBE-ß-CD: sulfobutyl-ß-cyclodextrin)
Before and after the administration, 1 ml of blood was taken from the jugular veins or
limb veins, and placed in an EDTAK2 centrifuge tube. Centrifugation was performed at
5000 rpm at 4° C. for 10 min, and plasma was collected. The blood collection time points
for groups G1 and G2 (the intravenous group and intragastric administration group) were:
0 min, 5 min, 15 min, 30 min, 1 h, 2 h, 4 h, 6 h, 8 h, 10 h, 12 h, 24 h, 48 h, and 72 h. The
blood collection time points for groups G3 and G4 (the intravenous group and intragastric
administration group) were: 0 min, 5 min, 15 min, 30 min, 1 h, 2 h, 4 h, 6 h, 8 h, 10 h, 12
h, and 24 h. Before analysis and detection, all samples were stored at -80° C. and the
samples were quantitatively analyzed by LC-MS/MS.

Conclusion: The compounds of the present disclosure, such as the compounds of the Examples, have good oral absorption performance in beagle dogs.

### 8. Pharmacokinetic Test in Monkeys

Experimental animals: male cynomolgus monkeys, 3-5 kg, 3-6 years old, 4-6 monkeys/compound. purchased from Suzhou Xishan Biotechnology Inc.

Experimental method: on the day of the test, 4-6 monkeys/compound were randomly grouped according to their body weights. The animals were fasted but with water available for 14 to 18 h one day before the administration and were fed 4 h after the administration.

TABLE 7
Administration information

| | Number | Administration information | | | | | |
|---|---|---|---|---|---|---|---|
| | | | Administration | Administration | Administration | | |
| | | Test | dosage | concentration | volume | Collected | Mode of |
| Group | Male | compound | (mg/kg) | (mg/mL) | (mL/kg) | samples | administration |
| G1 | 2 | Compound | 1 | 1 | 1 | Plasma | Intravenous |
| | | | | | | | administration |
| G2 | 2 | | 10 | 2 | 5 | Plasma | Intragastric |
| | | | | | | | administration |
| G3 | 3 | Compound | 1 | 1 | 1 | Plasma | Intravenous |
| | | | | | | | administration |
| G4 | 3 | Compound | 10 | 2 | 5 | Plasma | Intravenous |
| | | | | | | | administration |

Note: solvent for intravenous administration: 5% DMA + 5% Solutol + 90% Saline;
vehicle for intragastric administration: 5% DMSO+95% (20% SBE-B-CD in saline)
(DMA: dimethylacetamide; Solutol: polyethylene glycol-15-hydroxystearate; Saline:
physiological saline; DMSO: dimethyl sulfoxide; SBE-B-CD: sulfobutyl-ß-cyclodextrin)
*Dosage is calculated based on free base.

Before and after the administration, 1.0 mL of blood was taken from the limb veins and placed in an EDTAK2 centrifuge tube. Centrifugation was performed at 5000 rpm at 4° C. for 10 min, and the plasma was collected. The blood collection time points for the intravenous group and intragastric administration group were: 0 min, 5 min, 15 min, 30 min, 1 h, 2 h, 4 h, 6 h, 8 h, 10 h, 12 h, and 24 h. Before analysis and detection, all samples were stored at −80° C., and the samples were quantitatively analyzed by LC-MS/MS.

Conclusion: The compounds of the present disclosure, such as the compounds of the Examples, have good oral absorption performance in monkeys.

9. Test for hERG Potassium Ion Channel

Experimental platform: electrophysiological manual patch-clamp system

Cell line: Chinese hamster ovary (CHO) cell line stably expressing hERG potassium ion channel

Experimental method: in CHO (Chinese Hamster Ovary) cells stably expressing hERG potassium channel, the whole cell patch-clamp technique was used to record hERG potassium channel current at room temperature. The glass microelectrode was made of a glass electrode blank (BF150-86-10, Sutter) by a puller. The tip resistance after filling the liquid in the electrode was about 2-5 MΩ. The glass microelectrode can be connected to the patch-clamp amplifier by inserting the glass microelectrode into an amplifier probe. The clamping voltage and data recording were controlled and recorded by the pClamp 10 software through a computer. The sampling frequency was 10 kHz, and the filtering frequency was 2 kHz. After the whole cell records were obtained, the cells were clamped at −80 mV, and the step voltage that induced the hERG potassium current (I hERG) was depolarized from −80 mV to +20 mV for 2 s, then repolarized to −50 mV, and returned to −80 mV after 1 s. This voltage stimulation was given every 10 s, and the administration process was started after the hERG potassium current was confirmed to be stable (at least 1 minute). The compound was administered for at least 1 minute at each test concentration, and at least 2 cells (n≥2) were tested at each concentration.

Data processing: data analysis processing was carried out by using pClamp 10, GraphPad Prism 5 and Excel software. The inhibition degree of hERG potassium current (peak value of hERG tail current induced at −50 mV) at different compound concentrations was calculated by the following formula:

$$Inhibition \% = [ 1 - ( I / Io ) ] \times 100 \%$$

where Inhibition % represents the percentage of inhibition of hERG potassium current by the compound, and I and Io represent the amplitude of hERG potassium current after and before the administration, respectively.

Compound IC50 was calculated using GraphPad Prism 5 software by fitting according to the following equation:

$$Y = Bottom + ( Top - Bottom ) / ( 1 + 10 \hat{} ( ( Log {IC}_{50} - X ) \times HillSlope ) )$$

Among the equation, X represents the Log value of the tested concentration of the test sample, Y represents the inhibition percentage at the corresponding concentration, and Bottom and Top represent the minimum and maximum inhibition percentage, respectively.

Conclusion: The compounds of the present disclosure, for example, the compounds of the Examples, has no apparent inhibitory activity against hERG.

### 10. Test of Stability in Liver Microsomes

In this experiment, liver microsomes of three species, including monkey, rat and mouse, were used as in vitro models to evaluate the metabolic stability of the test compound.

At 37° C., 1 μM of the test compound was co-incubated with microsomal protein and coenzyme NADPH. At given time points of the reaction (5 min, 10 min, 20 min, 30 min, and 60 min), the reaction was terminated by adding ice-cold acetonitrile containing an internal standard. The LC-MS/MS method was used to measure the concentration of the test compound in the sample. T_{1/2} was calculated using the natural logarithm (ln) of the residual rate of the drug in the incubation system and the incubation time. In addition, the intrinsic clearance in liver microsomes CLint(mic) and the intrinsic clearance in liver CLint(Liver) were further calculated.

TABLE 8
Results of stability in liver microsomes

| | | | | Hepatic | |
|---|---|---|---|---|---|
| | | | Test compound | microsomal | Hepatic |
| | | | after 1 h | intrinsic | intrinsic |
| | | | incubation | clearance CLint | clearance |
| Test | | T1/2 | Remaining % | (mic) | CLint(liver) |
| compound | Species | (min) | (T = 60 min) | (μL/min/mg) | (mL/min/kg) |
| Compound 21- | Monkey | 53.6 | 45.1 | 25.8 | 34.9 |
| 2 | | | | | |
| Compound 35- | | 59.8 | 47.2 | 23.2 | 31.3 |
| 2 | | | | | |
| Compound 36- | | 18.9 | 11.6 | 73.5 | 99.2 |
| 2 | | | | | |
| Control | | 10.6 | 1.8 | 130 | 176 |
| compound A | | | | | |
| Compound 21- | Rat | 168 | 80.4 | 8.24 | 14.8 |
| 2 | | | | | |
| Compound 35- | | 128 | 74.5 | 10.8 | 19.5 |
| 2 | | | | | |
| Compound 36- | | 165 | 77.3 | 8.38 | 15.1 |
| 2 | | | | | |
| Control | | 35.7 | 31.9 | 38.8 | 69.9 |
| compound A | | | | | |
| Compound 21- | Mice | 92.8 | 63.7 | 14.9 | 59.1 |
| 2 | | | | | |
| Compound 35- | | >18 | 83.8 | <7.45 | <29.5 |
| 2 | | 6 | | | |
| Compound 36- | | 159 | 80.8 | 8.74 | 34.6 |
| 2 | | | | | |
| Control | | 49.3 | 42.6 | 28.1 | 111 |
| compound A | | | | | |

Conclusion: The compounds of the present disclosure, such as the compounds of the Examples, have good stability in the liver microsomes of monkeys, mice and rats.

1. A compound or a stereoisomer, mesomer/racemate, deuterated product, solvate, prodrug, metabolite, pharmaceutically acceptable salt, or co-crystal thereof, which compound is selected from a compound of general formula (I), wherein 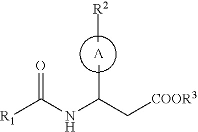
R¹ is selected from —CHR^{1a}R^{1b} or —NR^{1a}R^{1b};
R^{1a} is selected from C₁₋₆ alkyl, wherein the alkyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl;
R^{1b} is selected from C₄₋₁₀ carbocyclic ring or 5- to 10-membered heterocyclic ring, wherein the carbocyclic ring or heterocyclic ring is optionally substituted with 0 to 4 R^{b}, and the heterocyclic ring contains 1 to 4 heteroatoms selected from O, S or N;
each R^{b} is independently selected from deuterium, halogen, OH, ═O, cyano, COOH, NH₂, —C₀₋₄ alkyl-NHC₁₋₆ alkyl, —C₀₋₄ alkyl-N(C₁₋₆ alkyl)₂, C₁₋₆ alkyl, C₂₋₆ alkynyl, C₁₋₆ alkoxy, —C₀₋₄ alkyl-C₃₋₁₀ carbocyclic ring, —C₀₋₄ alkyl-3- to 10-membered heterocyclic ring or R^{ba}, wherein the alkyl, alkynyl, alkoxy, carbocyclic ring or heterocyclic ring is optionally substituted with 0 to 4 substituents selected from deuterium, halogen, OH, ═O, cyano, COOH, CONH₂, CONHC₁₋₆ alkyl, CON(C₁₋₆ alkyl)₂, NH₂, NHC₁₋₆ alkyl, N(C₁₋₆ alkyl)₂, N(C₁₋₆ alkyl)(C₃₋₆ cycloalkyl), NH(C₃₋₆ cycloalkyl), C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, and the heterocyclic ring contains 1 to 4 heteroatoms selected from O, S or N;
R^{ba} is selected from —C₀₋₄ alkyl-7- to 12-membered heterocyclic ring, —C₀₋₄ alkyl-4- to 6-membered heterocyclic ring connected via a carbon atom, 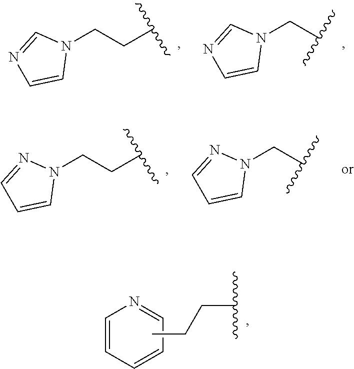
and the R^{ba} is optionally substituted with 1 to 4 substituents selected from H, halogen, OH, ═O, cyano, COOH, CONH₂, CONHC₁₋₆ alkyl, CON(C₁₋₆ alkyl)₂, NH₂, NHC₁₋₆ alkyl, N(C₁₋₆ alkyl)₂, C₁₋₆ alkyl, C₂₋₆ alkynyl, C₁₋₆ alkoxy, halogen-substituted C₁₋₆ alkyl, halogen-substituted C₁₋₆ alkoxy, C₁₋₆ alkoxyalkyl or R^{k}, wherein the heterocyclic ring contains 1 to 4 heteroatoms selected from O, S or N;
R^{k} is selected from —C₁₋₄ alkyl-NH₂, —C₁₋₄ alkyl-NHC₁₋₆ alkyl, —C₁₋₄ alkyl-N(C₁₋₆ alkyl)₂, —C₀₋₄ alkyl-C₃₋₁₀ carbocyclic ring or —C₀₋₄ alkyl-3- to 10-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy, halogen-substituted C₁₋₆ alkyl, halogen-substituted C₁₋₆ alkoxy or C₁₋₆ alkoxyalkyl, and the heterocyclic ring contains 1 to 4 heteroatoms selected from O, S or N;
R² is selected from C₁₋₆ alkyl, C₆₋₁₀ aromatic ring, 5- to 10-membered heteroaromatic ring, C₃₋₁₀ carbocyclic ring or 5- to 10-membered heterocyclic ring, and the R² is optionally substituted with 0 to 4 R^{2a}, wherein the heteroaromatic ring or heterocyclic ring contains 1 to 4 heteroatoms selected from O, S or N;
each R^{2a} is independently selected from deuterium, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl or C₁₋₆ alkoxy, and the heterocyclyl contains 1 to 3 heteroatoms selected from N, O or S;
as an option, R^{2a} and R^{2a} are directly linked to form C₄₋₇ carbocyclic ring or 4- to 7-membered heterocyclic ring, wherein the carbocyclic ring or heterocyclic ring is optionally substituted with 1 to 4 substituents selected from deuterium, halogen, OH, cyano, ═O, C₁₋₆ alkyl, C₂₋₆ alkenyl, C₂₋₆ alkynyl, C₁₋₆ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano;
R³ is selected from H or C₁₋₆ alkyl, wherein the alkyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ carbocyclic ring;
ring A is selected from C₈₋₁₀ fused carbocyclic ring, and the ring A is optionally substituted with 0 to 4 R^{a5};
or ring A is selected from 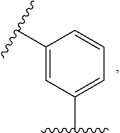 [SMILES: *c1cccc(*)c1]
and the ring A is substituted with one substituent selected from C₂₋₆ alkenyl or C₂₋₆ alkynyl and optionally substituted with 1 to 3 R^{a5};
R^{a5} is selected from halogen, OH, cyano, C₁₋₆ alkyl, C₂₋₆ alkenyl, C₂₋₆ alkynyl, C₁₋₆ alkoxy, C₃₋₆ cycloalkyl, or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₆ alkyl, N(C₁₋₆ alkyl)₂, C₁₋₆ alkyl, C₁₋₆ alkoxy or C₃₋₆ cycloalkyl, and the heterocyclyl contains 1 to 3 heteroatoms selected from N, O or S.

2. The compound or the stereoisomer, mesomer/racemate, deuterated product, solvate, prodrug, metabolite, pharmaceutically acceptable salt, or co-crystal thereof according to claim 1, wherein
R¹ is selected from 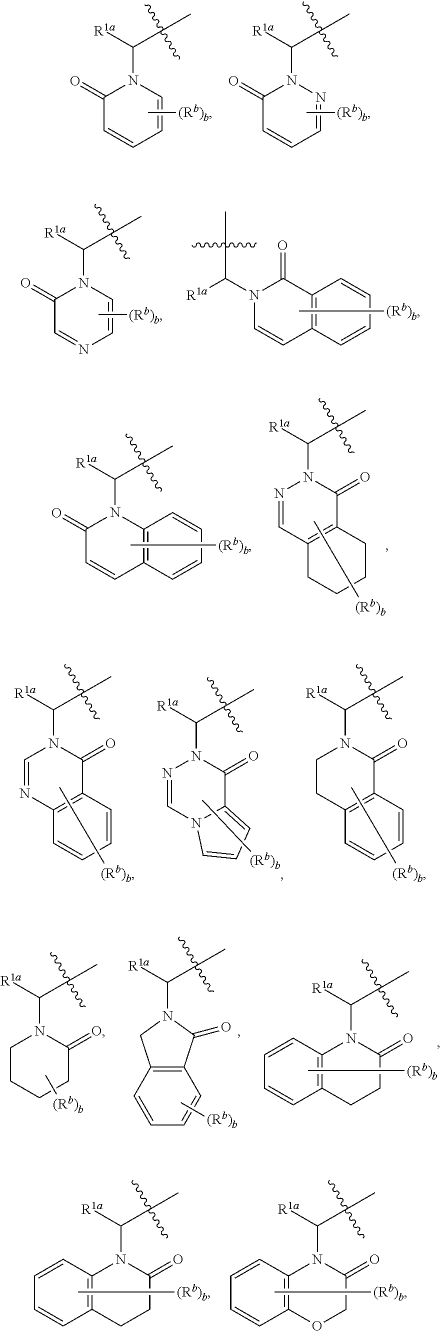 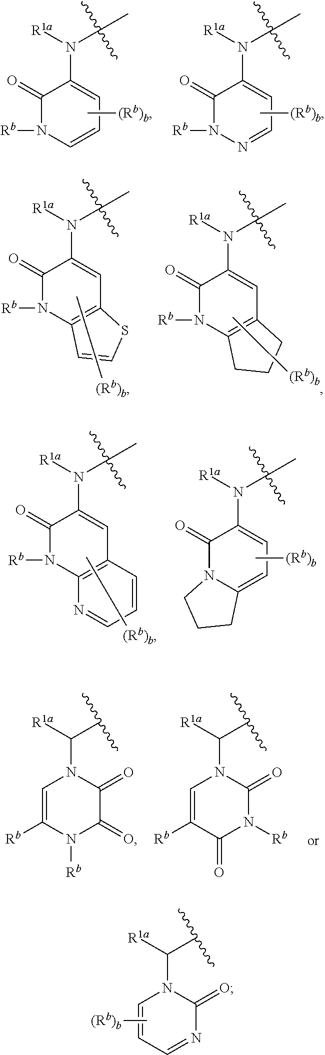
b is selected from 0, 1, 2 or 3;
each R^{b} is independently selected from deuterium, halogen, OH, ═O, cyano, COOH, NH₂, —C₀₋₄ alkyl-NHC₁₋₄ alkyl, —C₀₋₄ alkyl-N(C₁₋₄ alkyl)₂, C₁₋₄ alkyl, C₂₋₄ alkynyl, C₁₋₄ alkoxy, —C₀₋₄ alkyl-C₃₋₆ carbocyclic ring, —C₀₋₄ alkyl-3- to 7-membered heterocyclic ring or R^{ba}, wherein the alkyl, alkynyl, alkoxy, carbocyclic ring or heterocyclic ring is optionally substituted with 0 to 4 substituents selected from deuterium, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heterocyclic ring contains 1 to 4 heteroatoms selected from O, S or N;
R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl connected via a carbon atom, 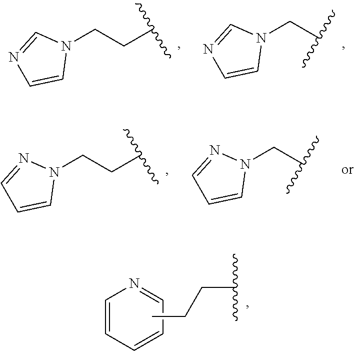
and the R^{ba} is optionally substituted with 1 to 4 substituents selected from H, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclic ring or —C₀₋₂ alkyl-3- to 6-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclic ring contains 1 to 4 heteroatoms selected from O, S or N;
R² is selected from C₁₋₄ alkyl, benzene ring, naphthalene ring, 5- to 6-membered heteroaromatic ring, 9- to 10-membered heteroaromatic ring, C₃₋₁₀ non-aromatic carbocyclic ring, 5- to 10-membered non-aromatic heterocyclic ring, benzo C₄₋₆ carbocyclyl or benzo 4- to 6-membered heterocyclyl, and the R₂ is optionally substituted with 0 to 4 R^{2a}, wherein the heteroaromatic ring or heterocyclic ring contains 1 to 4 heteroatoms selected from O, S or N;
each R^{2a} is independently selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl, or heterocyclyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl or C₁₋₄ alkoxy, and the heterocyclyl contains 1 to 3 heteroatoms selected from N, O or S;
as an option, R^{2a} and R^{2a} are directly linked to form C₄₋₇ carbocyclic ring or 4- to 7-membered heterocyclic ring, wherein the carbocyclic ring or heterocyclic ring is optionally substituted with 1 to 4 substituents selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano;
R³ is selected from H or C₁₋₄ alkyl, wherein the alkyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy or C₃₋₆ carbocyclic ring;
ring A is selected from benzo C₄₋₆ carbocyclic ring, and the ring A is optionally substituted with 0 to 4 R^{a5};
or ring A is selected from 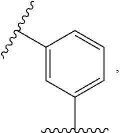 [SMILES: *c1cccc(*)c1]
and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, and the heterocyclyl contains 1 to 3 heteroatoms selected from N, O or S.

3. The compound or the stereoisomer, mesomer/racemate, deuterated product, solvate, prodrug, metabolite, pharmaceutically acceptable salt, or co-crystal thereof according to claim 2, wherein
each R^{b} is independently selected from deuterium, halogen, OH, ═O, cyano, C₁₋₄ alkyl, C₂₋₄ alkynyl, C₁₋₄ alkoxy, phenyl, 5- to 6-membered heteroaryl, —CH₂NHC₁₋₄ alkyl, —CH₂N(C₁₋₄ alkyl)₂, —CH₂CH₂—NHC₁₋₄ alkyl, —CH₂CH₂—N(C₁₋₄ alkyl)₂, phenyl, 5- to 6-membered heteroaryl, C₃₋₆ cycloalkyl, 3- to 7-membered heterocycloalkyl, —CH₂-phenyl, —CH₂-5- to 6-membered heteroaryl, —CH₂—C₃₋₆ cycloalkyl, —CH₂-3- to 7-membered heterocycloalkyl, —CH₂CH₂-phenyl, —CH₂CH₂-5- to 6-membered heteroaryl, —CH₂CH₂—C₃₋₆ cycloalkyl, —CH₂CH₂-3- to 7-membered heterocycloalkyl or R^{ba}, wherein the CH₂, alkyl, alkynyl, phenyl, heteroaryl, cycloalkyl or heterocycloalkyl is optionally substituted with 0 to 4 substituents selected from deuterium, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{ba} is selected from 7- to 8-membered monocyclic heterocycloalkyl, 7- to 11-membered spiro heterocycloalkyl, 7- to 11-membered bridged heterocycloalkyl, 4- to 6-membered monocyclic heterocycloalkyl connected via a carbon atom, —CH₂-7- to 8-membered monocyclic heterocycloalkyl, —CH₂-7- to 11-membered spiro heterocycloalkyl, —CH₂-7- to 11-membered bridged heterocycloalkyl, —CH₂-4- to 6-membered monocyclic heterocycloalkyl connected via a carbon atom, —CH₂CH₂-7- to 8-membered monocyclic heterocycloalkyl, —CH₂CH₂-7- to 11-membered spiro heterocycloalkyl, —CH₂CH₂-7- to 11-membered bridged heterocycloalkyl, —CH₂CH₂-4- to 6-membered monocyclic heterocycloalkyl connected via a carbon atom, 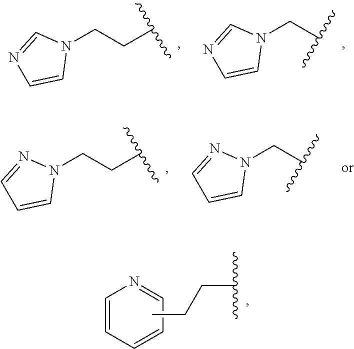
and the R^{ba} is optionally substituted with 1 to 4 substituents selected from H, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₄ alkyl-NH₂, —C₁₋₄ alkyl-NHC₁₋₄ alkyl, —C₁₋₄ alkyl-N(C₁₋₄ alkyl)₂, —C₀₋₄ alkyl-C₃₋₆ carbocyclic ring or —C₀₋₄ alkyl-3- to 6-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclic ring contains 1 to 4 heteroatoms selected from O, S or N;
R² is selected from benzene ring, naphthalene ring, 5- to 6-membered heteroaromatic ring, 9- to 10-membered heteroaromatic ring, C₃₋₆ cycloalkyl, 3- to 7-membered heterocycloalkyl, benzo C₄₋₆ carbocyclyl or benzo 4- to 6-membered heterocyclyl, and the R² is optionally substituted with 0 to 4 R^{2a}, wherein the heteroaromatic ring or heterocycloalkyl contains 1 to 4 heteroatoms selected from O, S or N;
each R^{2a} is independently selected from deuterium, halogen, OH, cyano, ═O, C₁₋₄ alkyl, C₂₋₄ alkenyl, C₂₋₄ alkynyl, C₁₋₄ alkoxy or C₃₋₆ cycloalkyl, wherein the alkyl, alkenyl, alkynyl, alkoxy or cycloalkyl is optionally substituted with 0 to 4 substituents selected from deuterium, halogen, OH, ═O, cyano, C₁₋₄ alkyl or C₁₋₄ alkoxy;
as an option, R^{2a} and R^{2a} are directly linked to form 4-membered carbocyclic ring, 5-membered carbocyclic ring, 6-membered carbocyclic ring, 4-membered heterocyclic ring, 5-membered heterocyclic ring or 6-membered heterocyclic ring, wherein the carbocyclic ring or heterocyclic ring is optionally substituted with 1 to 4 substituents selected from deuterium, halogen, OH, cyano, ═O, C₁₋₄ alkyl, C₂₋₄ alkenyl, C₂₋₄ alkynyl, C₁₋₄ alkoxy, C₃₋₆ cycloalkyl or 3- to 7-membered heterocycloalkyl, and the alkyl, alkenyl, alkynyl, alkoxy, cycloalkyl or heterocycloalkyl is optionally substituted with 1 to 4 substituents selected from deuterium, halogen, OH or cyano;
R³ is selected from H or C₁₋₄ alkyl, wherein the alkyl is optionally substituted with 0 to 4 substituents selected from deuterium, halogen, C₁₋₄ alkyl, C₁₋₄ alkoxy or benzene ring;
ring A is selected from 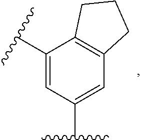 [SMILES: *c1cc(*)c2c(c1)CCC2]
and the ring A is optionally substituted with 0 to 4 R^{a5};
or ring A is selected from 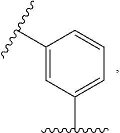 [SMILES: *c1cccc(*)c1]
and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkynyl, C₁₋₄ alkoxy or C₃₋₆ cycloalkyl, wherein the alkyl, alkynyl, alkoxy, cycloalkyl or heterocyclyl is optionally substituted with 0 to 4 substituents selected from deuterium, halogen, OH, ═O, cyano, C₁₋₄ alkyl, C₁₋₄ alkoxy or C₃₋₆ cycloalkyl.

4. The compound or the stereoisomer, mesomer/racemate, deuterated product, solvate, prodrug, metabolite, pharmaceutically acceptable salt, or co-crystal thereof according to claim 3, wherein
R^{1a} is selected from methyl, ethyl, propyl, butyl, isobutyl, sec-butyl, tert-butyl, —CH₂-cyclopropyl or —CH₂-cyclobutyl;
each R^{b} is independently selected from deuterium, F, Cl, Br, I, OH, ═O, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azolidinyl, azinanyl, oxetanyl, oxolanyl, oxanyl, morpholinyl, phenyl, pyridine, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azolidinyl, —CH₂-azinanyl, —CH₂-oxetanyl, —CH₂-oxolanyl, —CH₂-oxanyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl, —CH₂CH₂-morpholinyl, —CH₂-phenyl, —CH₂-pyridyl, —CH₂CH₂-phenyl or —CH₂CH₂-pyridyl, which, when substituted, is substituted with 0 to 4 substituents selected from deuterium, halogen, OH, ═O, cyano, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
R^{ba} is selected from one of the following optionally substituted groups: 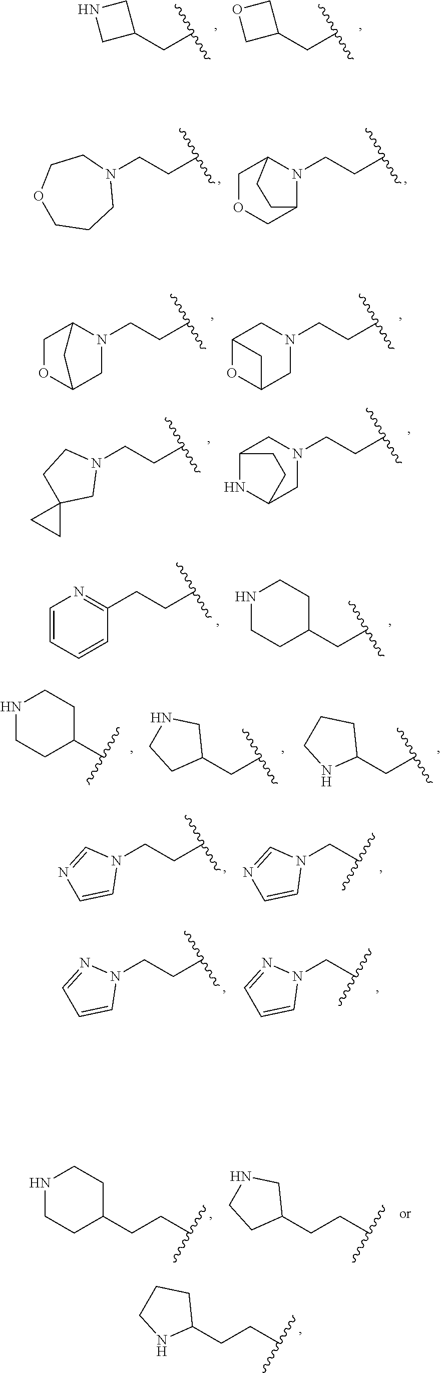
which, when substituted, is optionally substituted with 1 to 4 substituents selected from deuterium, halogen, OH, ═O, cyano, CONH₂, CONHC₁₋₄ alkyl, CON(C₁₋₄ alkyl)₂, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k};
R^{k} is selected from —CH₂N(CH₃)₂, —CH₂-cyclopropyl, —CH₂-cyclobutyl, cyclopropyl, cyclobutyl, cyclopentyl, cyclohexyl, azetidinyl, azolidinyl, azinanyl or oxetanyl, wherein the cyclopropyl, cyclobutyl, cyclopentyl, cyclohexyl, azetidinyl, azolidinyl or azinanyl is optionally substituted with 1 to 4 substituents selected from deuterium, halogen, OH, ═O, cyano, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₁₋₄ alkoxy, halogen-substituted C₁₋₄ alkyl, halogen-substituted C₁₋₄ alkoxy or C₁₋₄ alkoxyalkyl;
R² is selected from benzene ring, pyridyl, pyridonyl, pyrazinyl, pyrimidyl, thienyl, thiazolyl, furyl, oxazolyl, pyrrolyl, pyrazolyl, imidazolyl, cyclopropyl, cyclobutyl, cyclopentyl, cyclohexyl, azetidinyl, azolidinyl, azinanyl, oxetanyl, oxolanyl, oxanyl, morpholinyl, 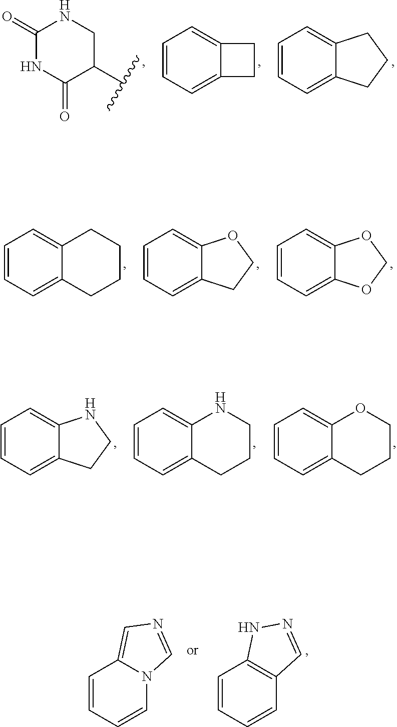
and the R² is optionally substituted with 0 to 4 R^{2a};
each R^{2a} is independently selected from deuterium, F, Cl, Br, I, OH, ═O, cyano, methyl, ethyl, ethenyl, ethynyl, methoxy, ethoxy, cyclopropyl or cyclobutyl, wherein the methyl, ethyl, ethenyl, ethynyl, methoxy, ethoxy, cyclopropyl or cyclobutyl is optionally substituted with 0 to 4 substituents selected from deuterium, halogen, OH, ═O, cyano, C₁₋₄ alkyl or C₁₋₄ alkoxy;
R³ is selected from H, methyl, ethyl, propyl, butyl, isobutyl, sec-butyl, tert-butyl or benzyl;
ring A is selected from 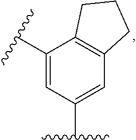 [SMILES: *c1cc(*)c2c(c1)CCC2]
and the ring A is optionally substituted with 0 to 4 R^{a5};
or ring A is selected from 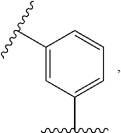 [SMILES: *c1cccc(*)c1]
and the ring A is substituted with one substituent selected from ethenyl, ethynyl, propynyl or propargyl and optionally substituted with 1 to 3 substituents selected from R^{a5};
each R^{a5} is independently selected from F, Cl, Br, I, OH, CN, ethynyl, propynyl, propargyl, methyl, ethyl, cyclopropyl, methoxy or ethoxy, wherein the ethynyl, propynyl, propargyl, methyl, ethyl, cyclopropyl, methoxy or ethoxy is optionally substituted with 1 to 4 substituents selected from deuterium, halogen, OH, ═O, cyano, C₁₋₄ alkyl, C₁₋₄ alkoxy or C₃₋₆ cycloalkyl.

5. The compound or the stereoisomer, mesomer/racemate, deuterated product, solvate, prodrug, metabolite, pharmaceutically acceptable salt, or co-crystal thereof according to claim 4, wherein
R¹ is selected from 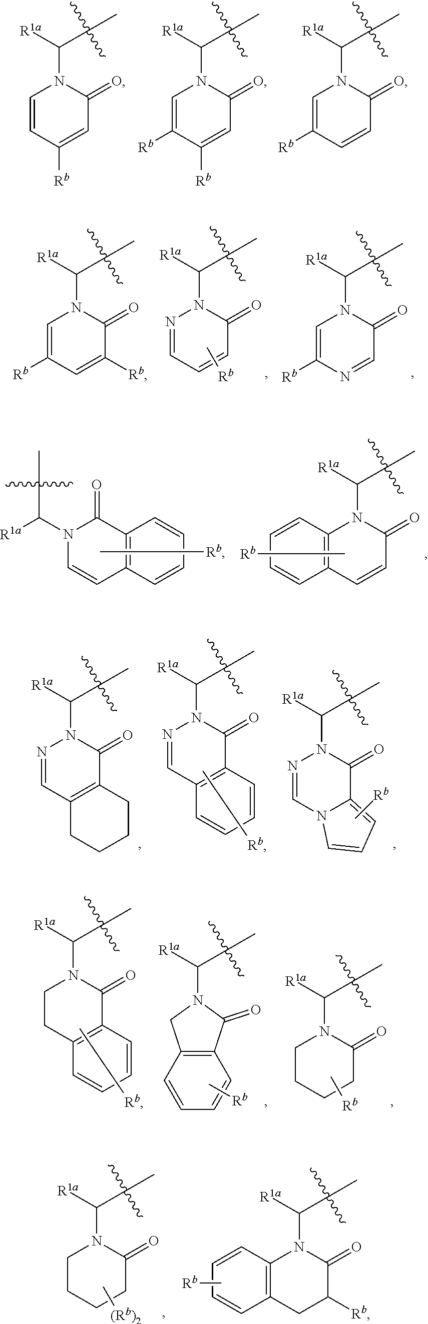 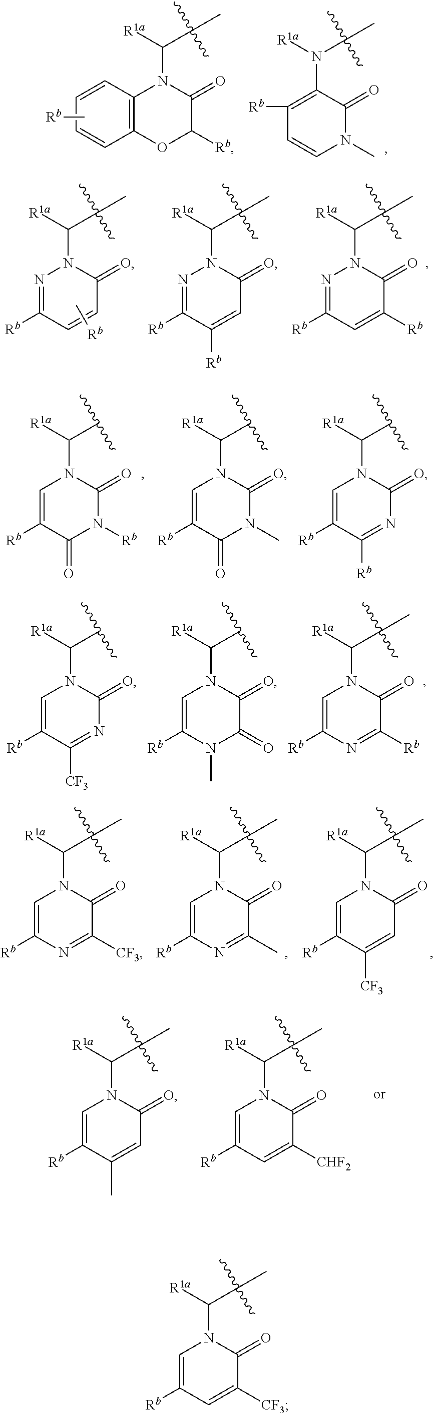
each R^{b} is independently selected from deuterium, F, Cl, Br, OH, cyano or R^{ba}, or each R^{b} is independently selected from one of the following substituted or unsubstituted groups: methyl, ethyl, ethynyl, methoxy, ethoxy, cyclopropyl, cyclobutyl, cyclopentyl, cyclohexyl, azetidinyl, azolidinyl, azinanyl, oxetanyl, oxolanyl, oxanyl, morpholinyl, phenyl, pyridyl, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂-cyclopropyl, —CH₂-cyclobutyl, —CH₂-cyclopentyl, —CH₂-cyclohexyl, —CH₂-azetidinyl, —CH₂-azolidinyl, —CH₂-azinanyl, —CH₂-oxetanyl, —CH₂-oxolanyl, —CH₂-oxanyl, —CH₂-morpholinyl, —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl, —CH₂CH₂-morpholinyl, —CH₂-phenyl, —CH₂-pyridyl, —CH₂CH₂-phenyl, —CH₂CH₂-pyridyl or 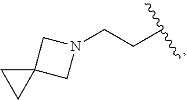 [SMILES: *CCN1CC2(CC2)C1]
which, when substituted, is substituted with 0 to 4 substituents selected from deuterium, F, Cl, Br, OH, ═O, cyano, CONH₂, CONHCH₃, CON(CH₃)₂, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{ba} is selected from one of the following optionally substituted groups: 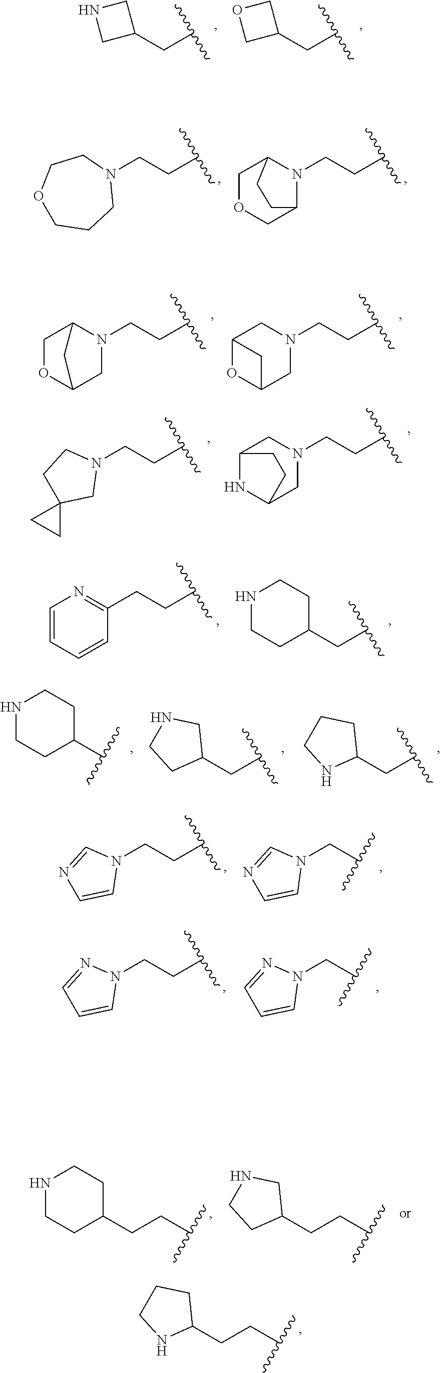
which, when substituted, is optionally substituted with 1 to 4 substituents selected from deuterium, F, Cl, Br, OH, ═O, cyano, CONH₂, CONHCH₃, CON(CH₃)₂, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k};
R^{k} is selected from —CH₂N(CH₃)₂, —CH₂-cyclopropyl, —CH₂-cyclobutyl, cyclopropyl, cyclobutyl, cyclopentyl, cyclohexyl, azetidinyl, azolidinyl, azinanyl or oxetanyl, wherein the cyclopropyl, cyclobutyl, cyclopentyl, cyclohexyl, azetidinyl, azolidinyl, azinanyl or oxetanyl is optionally substituted with 1 to 4 substituents selected from deuterium, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, methoxy, ethoxy, methoxymethyl, ethoxymethyl or methoxyethyl;
R² is selected from phenyl, pyridyl, pyridonyl, azolidinyl, morpholinyl or 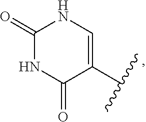 [SMILES: *c1c[nH]c(=O)[nH]c1=O]
and the R² is optionally substituted with 0 to 4 substituents selected from deuterium, F, Cl, Br, OH, CF₃, cyano, methyl, ethyl, methoxy, ethoxy, cyclopropyl or cyclobutyl;
or R² is selected from 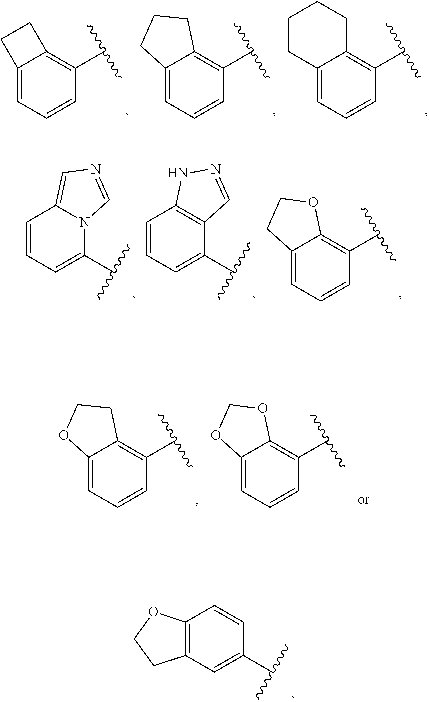
and the R² is optionally substituted with 1 to 4 substituents selected from deuterium, CD₃, —OCD₃, F, Cl, Br, OH, CF₃, cyano, methyl, ethyl, methoxy, ethoxy, cyclopropyl or cyclobutyl;
ring A is selected from

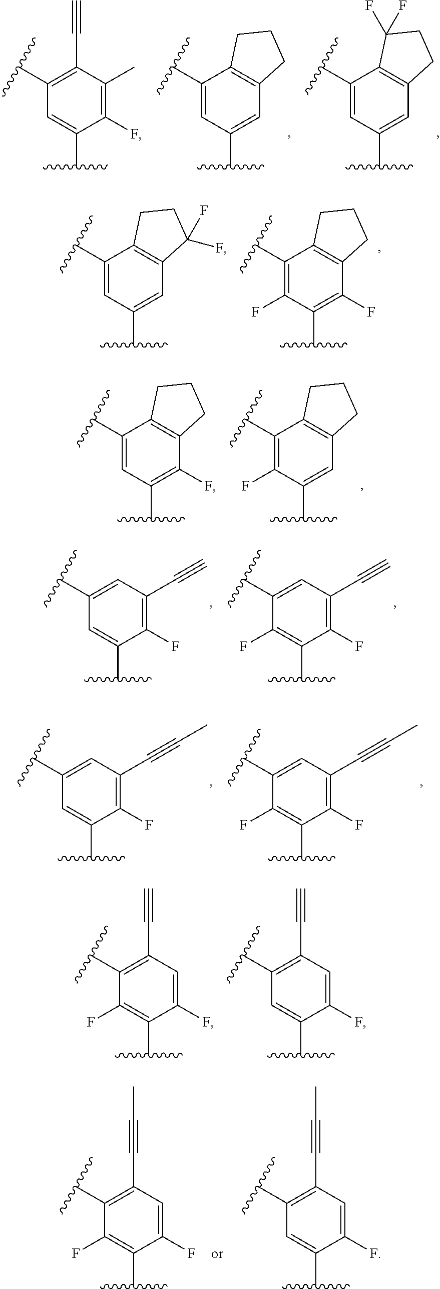

6. The compound or the stereoisomer, mesomer/racemate, deuterated product, solvate, prodrug, metabolite, pharmaceutically acceptable salt, or co-crystal thereof according to claim 5, wherein
R¹ is selected from 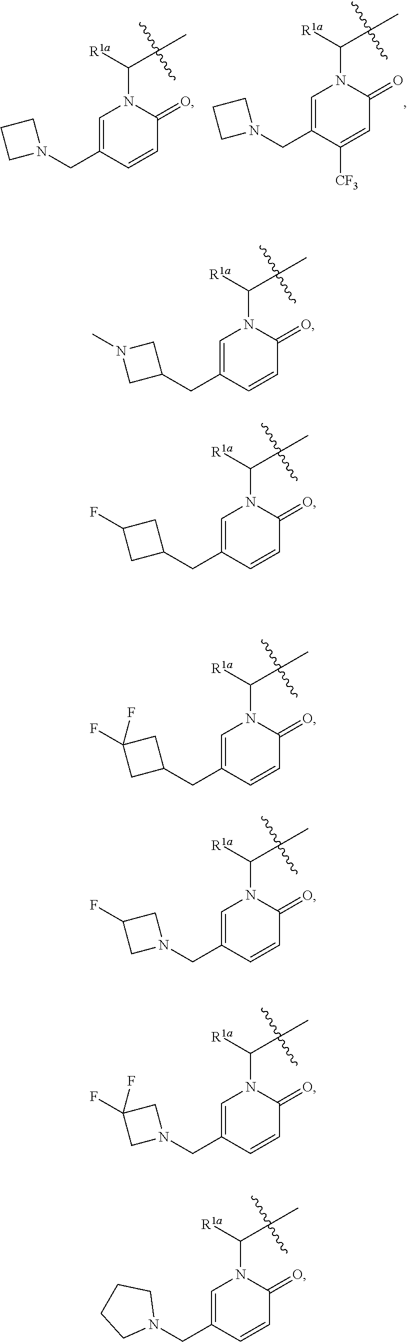 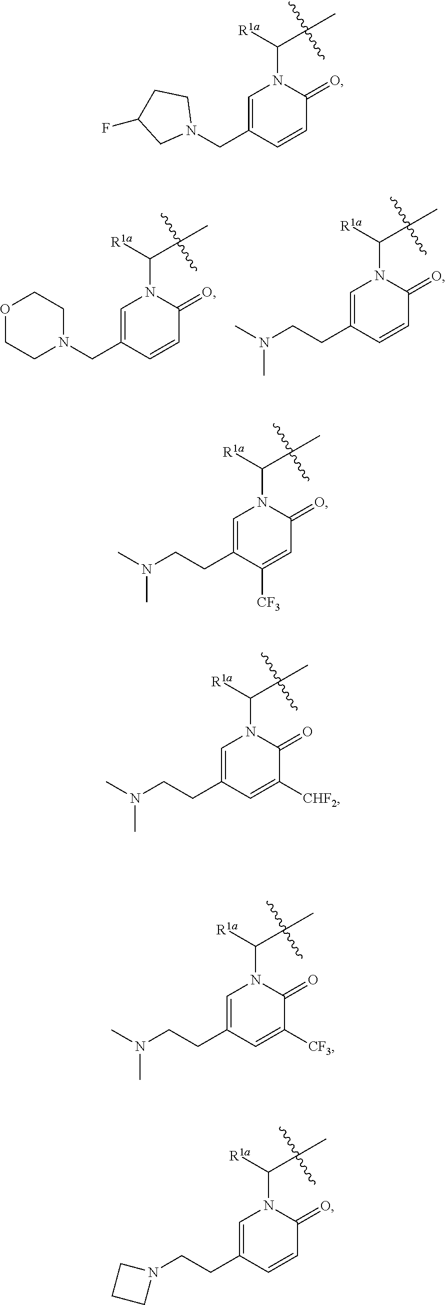 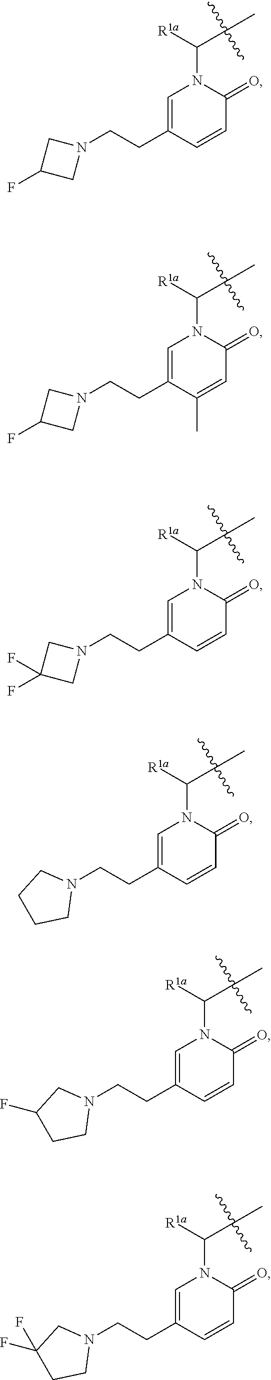 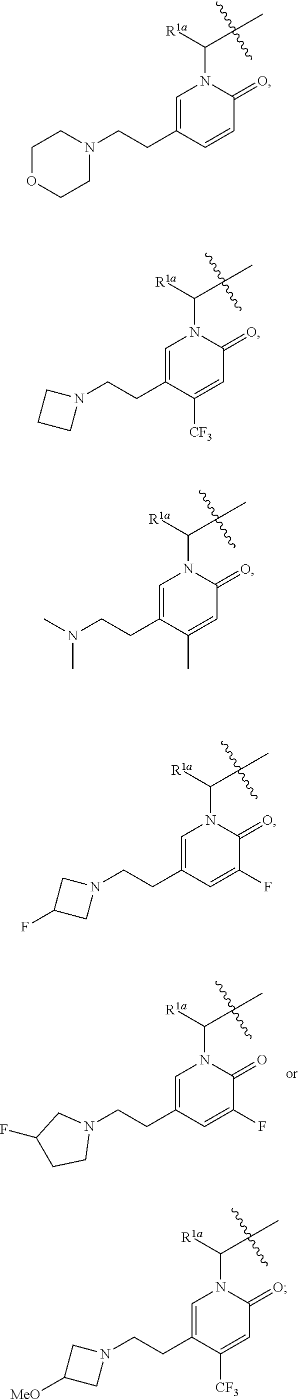
or R¹ is selected from 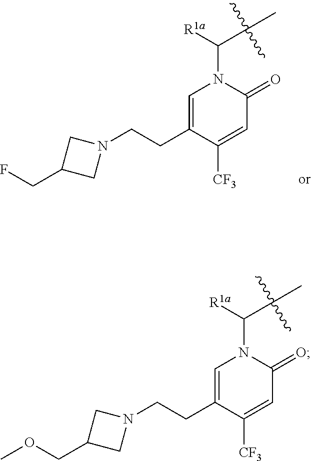
or R¹ is selected from 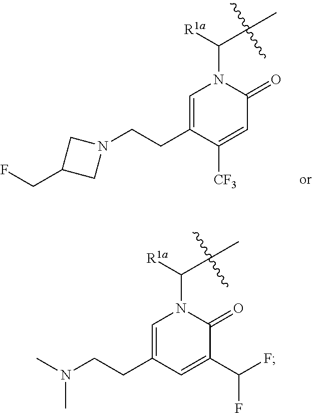
or R¹ is selected from 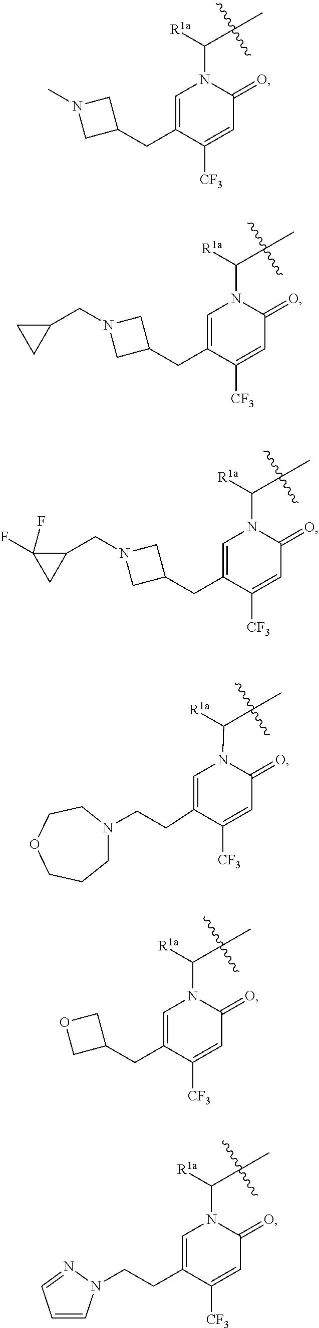 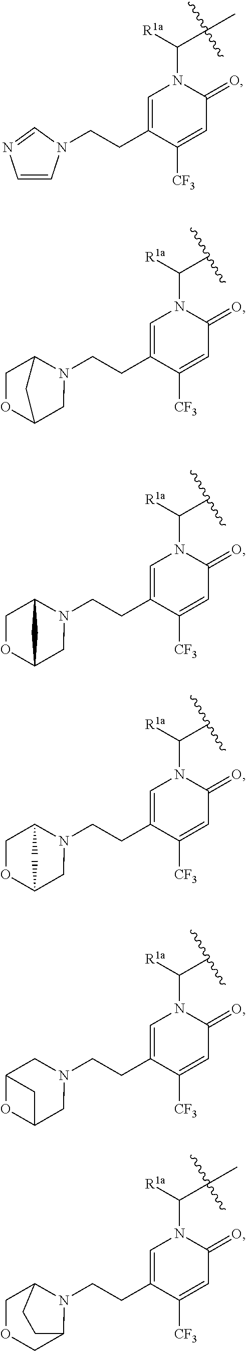 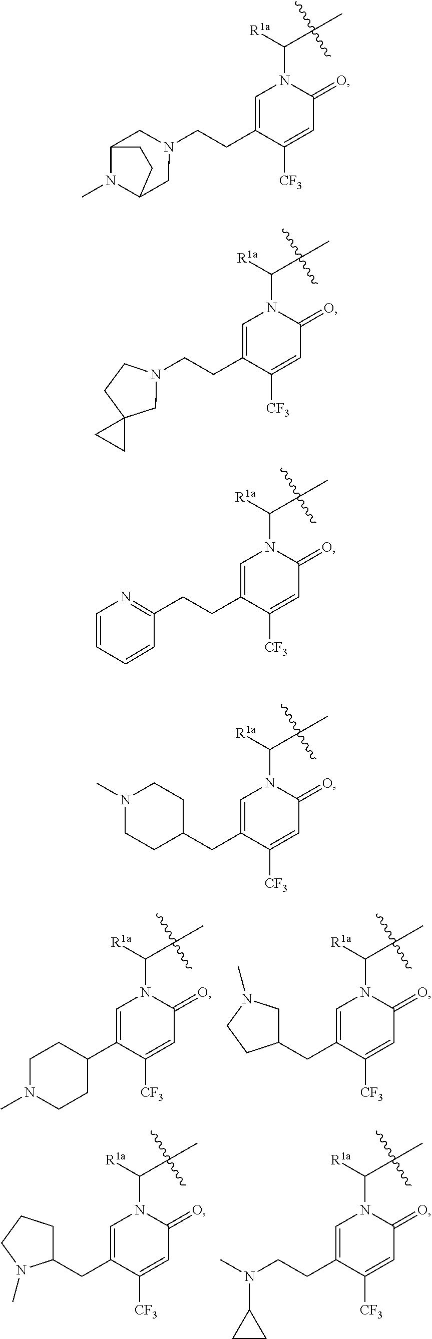 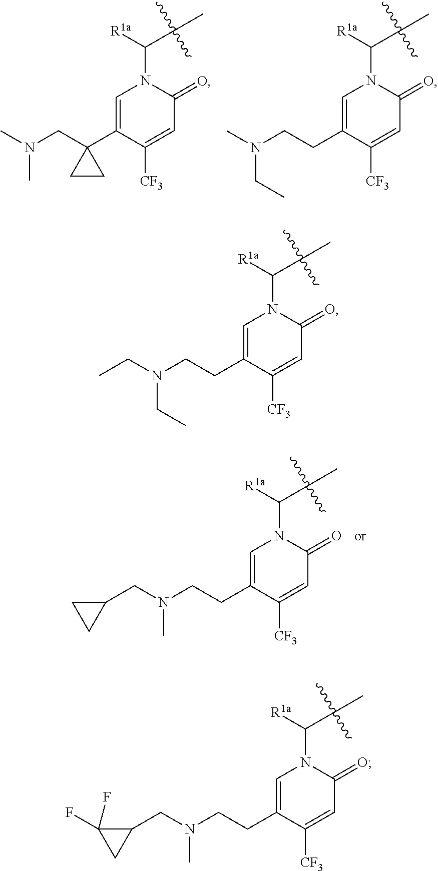
or R¹ is selected from 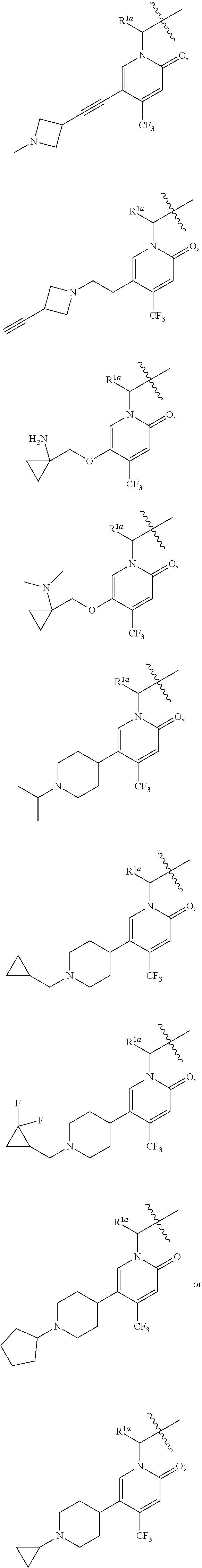
or R¹ is selected from 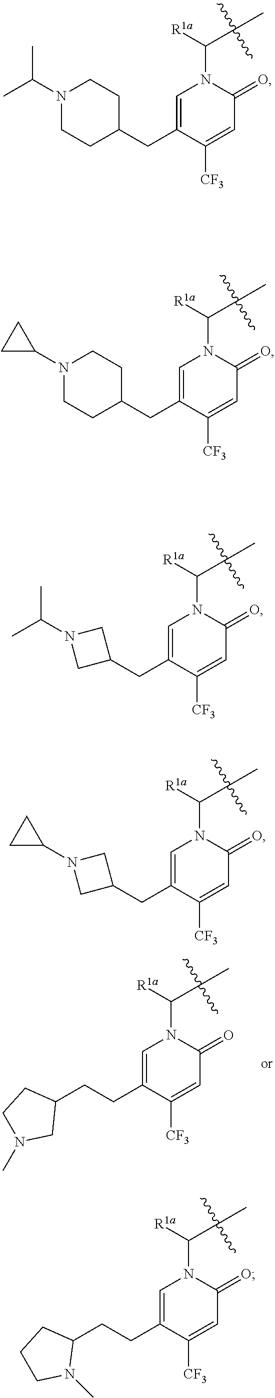
or R¹ is selected from 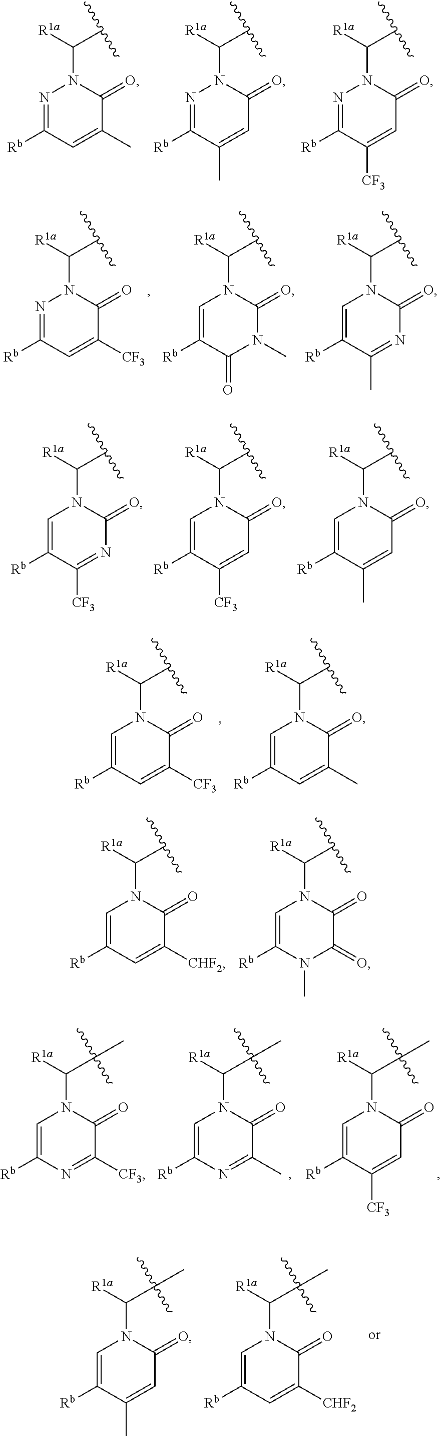 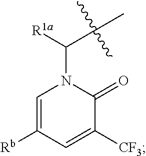
R² is selected from 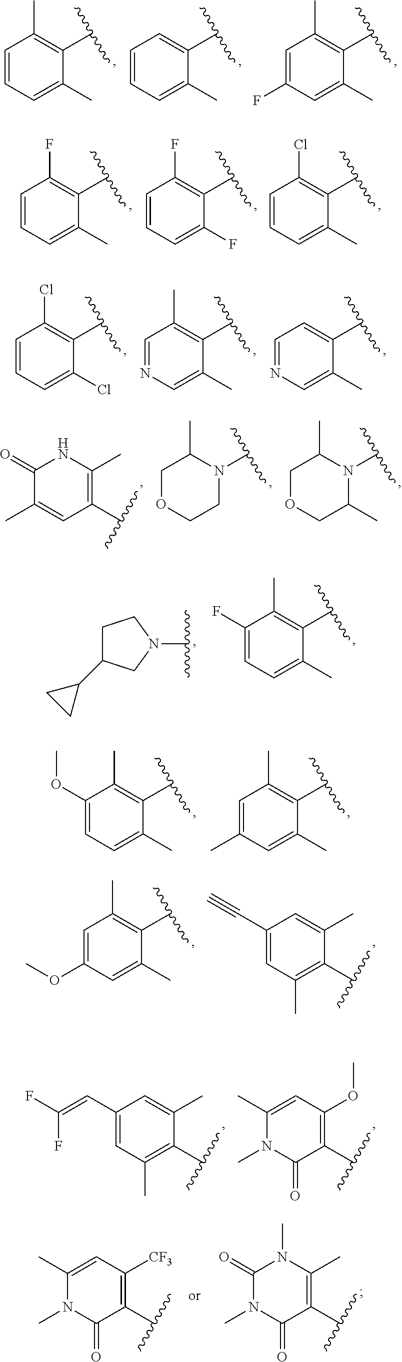
or R² is selected from 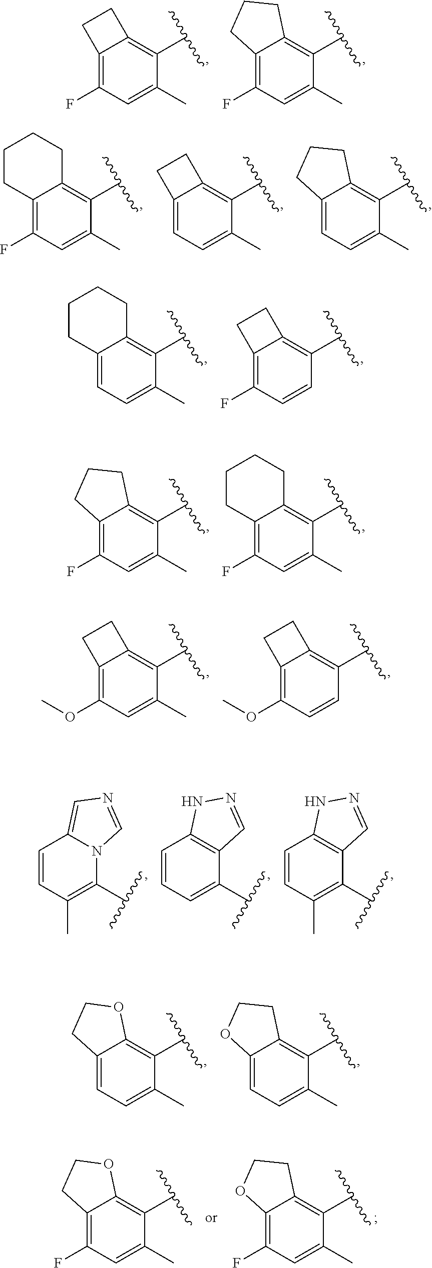
or R² is selected from 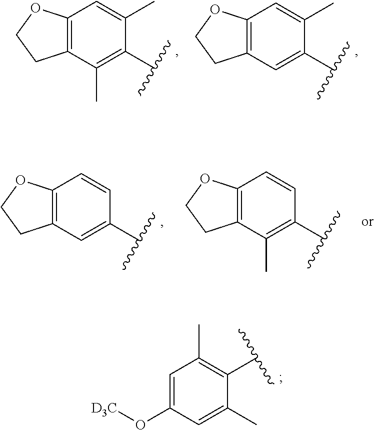 [SMILES: *c1c(C)cc2c(c1C)CCO2 ; *c1cc2c(cc1C)OCC2 ; *c1ccc2c(c1)CCO2 ; *c1ccc2c(c1C)CCO2 ; *c1c(C)cc(OC([2H])([2H])[2H])cc1C]
R^{1a} is selected from

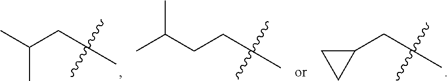

7. The compound or the stereoisomer, mesomer/racemate, deuterated product, solvate, prodrug, metabolite, pharmaceutically acceptable salt, or co-crystal thereof according to claim 1, wherein the compound is selected from a compound of general formula (I-a), (I-b), (I-a-1) or (I-b-1), 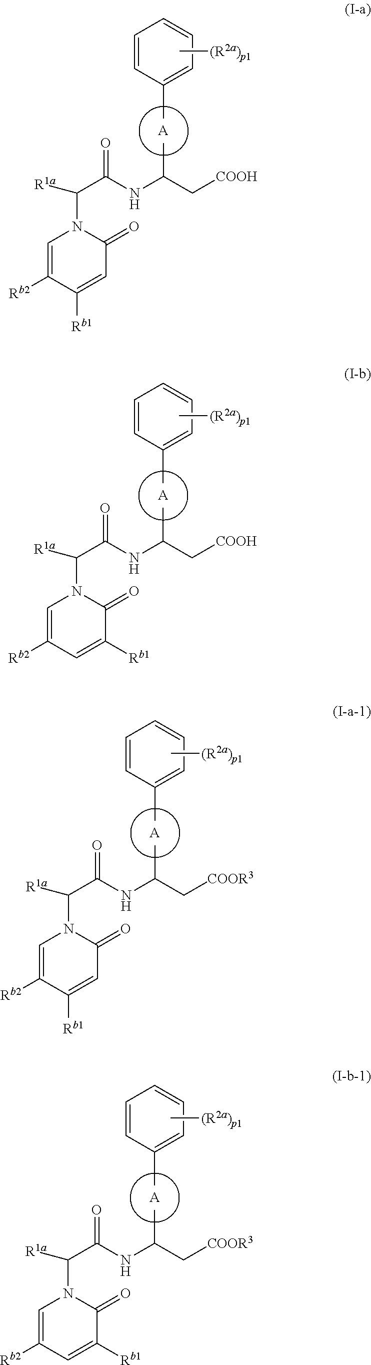
ring A is selected from benzo C₄₋₆ carbocyclic ring, and the ring A is optionally substituted with 0 to 4 R^{a5};
or ring A is selected from 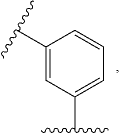 [SMILES: *c1cccc(*)c1]
and the ring A is substituted with one substituent selected from C₂₋₄ alkenyl or C₂₋₄ alkynyl and optionally substituted with 1 to 3 substituents selected from R^{a5};
R^{a5} is selected from halogen, OH, cyano, C₁₋₄ alkyl, C₂₋₄ alkenyl, C₂₋₄ alkynyl, C₁₋₄ alkoxy, C₃₋₆ cycloalkyl or 3- to 7-membered heterocyclyl, wherein the alkyl, alkenyl, alkynyl, alkoxy, cycloalkyl or heterocyclyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, halogen-substituted C₁₋₄ alkyl, cyano-substituted C₁₋₄ alkyl, C₁₋₄ alkoxy or C₃₋₆ cycloalkyl, and the heterocyclyl contains 1 to 3 heteroatoms selected from N, O or S;
R³ is selected from H or C₁₋₄ alkyl, wherein the alkyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy or C₃₋₆ carbocyclic ring;
p1 is selected from 0, 1, 2, 3 or 4;
R^{2a} is selected from deuterium, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from deuterium, halogen, OH, CN, C₁₋₄ alkyl or C₁₋₄ alkoxy, and the heterocycloalkyl contains 1 to 4 heteroatoms selected from O, S or N;
R^{1a} is selected from 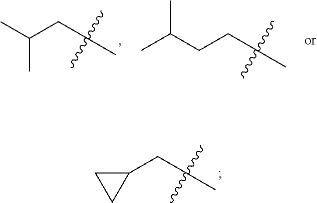
R^{b1} is selected from H, C₁₋₄ alkyl or C₃₋₆ cycloalkyl, wherein the alkyl or cycloalkyl is optionally substituted with 0 to 4 substituents selected from deuterium, halogen, OH, CN, NHC₁₋₄ alkyl, N(C₁₋₄ alkyl)₂, C₁₋₄ alkyl or C₁₋₄ alkoxy;
R^{b2} is selected from H, R^{ba}, or one of the following substituted or unsubstituted groups: C₁₋₄ alkyl, C₂₋₄ alkynyl, C₁₋₄ alkoxy, phenyl, 5- to 6-membered heteroaryl, —CH₂CH₂-phenyl, —CH₂CH₂-5- to 6-membered heteroaryl, —CH₂CH₂—NHC₁₋₄ alkyl, —CH₂CH₂—N(C₁₋₄ alkyl)₂, —CH₂CH₂—C₃₋₆ cycloalkyl or —CH₂CH₂-3- to 7-membered heterocycloalkyl, wherein the CH₂, alkyl, cycloalkyl or heterocycloalkyl is optionally substituted with 0 to 4 substituents selected from deuterium, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl connected via a carbon atom, 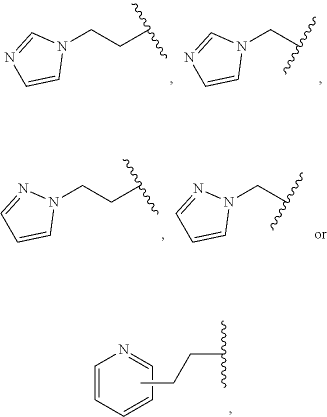
and the R^{ba} is optionally substituted with 1 to 4 substituents selected from H, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclic ring or —C₀₋₂ alkyl-3- to 6-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclic ring contains 1 to 4 heteroatoms selected from O, S or N.

8. The compound or the stereoisomer, mesomer/racemate, deuterated product, solvate, prodrug, metabolite, pharmaceutically acceptable salt, or co-crystal thereof according to claim 7, wherein
R^{2a} is selected from deuterium, —CD₃, —OCD₃, F, Cl, Br, I, OH, cyano, methyl, ethyl, methoxy or ethoxy;
R^{b1} is selected from H, F, CH₂F, CHF₂, CF₃, methyl or —CH₂CH₂N(CH₃)₂;
R^{b2} is selected from H, R^{ba}, or one of the following substituted or unsubstituted groups: methyl, ethyl, ethynyl, methoxy, ethoxy, —CH₂NH(CH₂CH₃), —CH₂N(CH₂CH₃)₂, —CH₂CH₂NH(CH₃), —CH₂CH₂N(CH₃)₂, —CH₂CH₂NH(CH₂CH₃), —CH₂CH₂N(CH₂CH₃)₂, —CH₂CH₂N(CH₃)(CH₂CH₃), —CH₂CH₂-cyclopropyl, —CH₂CH₂-cyclobutyl, —CH₂CH₂-cyclopentyl, —CH₂CH₂-cyclohexyl, —CH₂CH₂-azetidinyl, —CH₂CH₂-azolidinyl, —CH₂CH₂-azinanyl, —CH₂CH₂-oxetanyl, —CH₂CH₂-oxolanyl, —CH₂CH₂-oxanyl, —CH₂CH₂-morpholinyl, —CH₂CH₂-phenyl or —CH₂CH₂-pyridyl, which, when substituted, is substituted with 1, 2 or 3 substituents selected from H, F, Cl, Br, OH, ═O, cyano, NH₂, NHCH₃, N(CH₃)₂, N(CH₃)(cyclopropyl), NHCH₂CH₃, N(CH₂CH₃)₂, CH₂F, CHF₂, CF₃, methyl, ethyl, isopropyl, ethynyl, methoxy, ethoxy, methoxymethyl, ethoxymethyl, methoxyethyl or R^{k}.

9. The compound or the stereoisomer, mesomer/racemate, deuterated product, solvate, prodrug, metabolite, pharmaceutically acceptable salt, or co-crystal thereof according to claim 1, wherein the compound is selected from a compound of general formula (I-f), (I-g), (I-f-1), (I-g-1), (I-h), (I-i), (I-h-1) or (I-i-1), 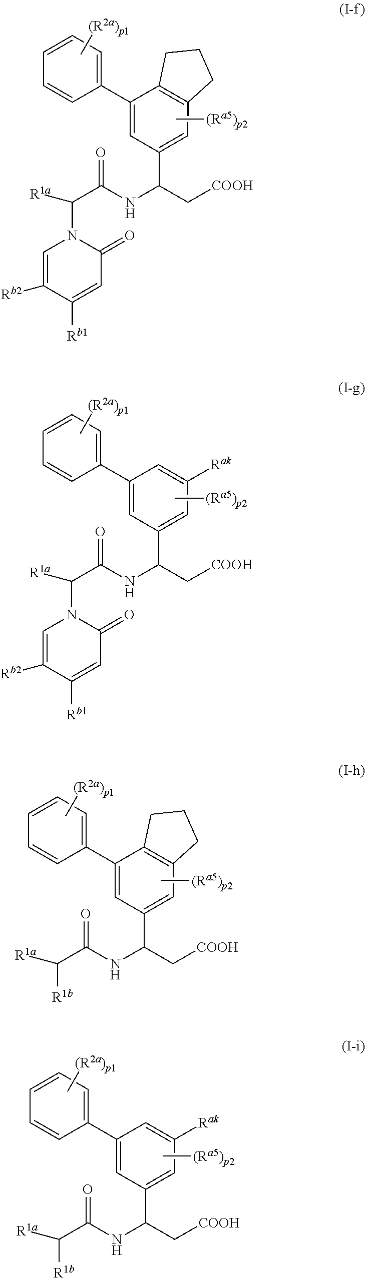 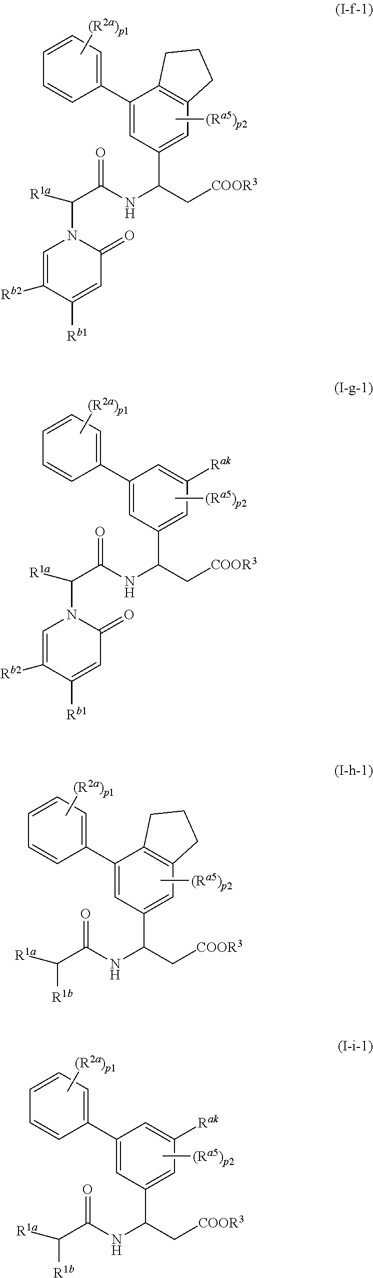
R^{1b} is selected from 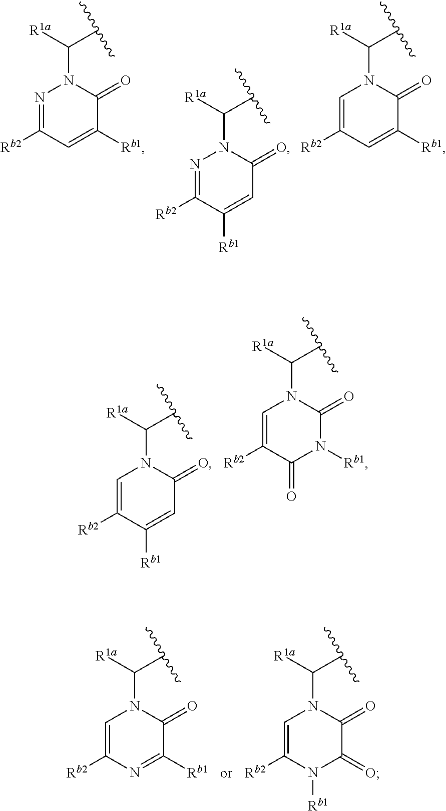
wherein p2 is selected from 0, 1 or 2;
p1 is selected from 0, 1, 2, 3 or 4;
R^{ak} is selected from C₂₋₄ alkynyl;
R^{2a} is selected from deuterium, halogen, OH, CN, C₁₋₄ alkyl, C₁₋₄ alkoxy, C₃₋₆ cycloalkyl or 4- to 6-membered heterocycloalkyl, wherein the alkyl, alkoxy, cycloalkyl or heterocycloalkyl is optionally substituted with 0 to 4 substituents selected from deuterium, halogen, OH, CN, C₁₋₄ alkyl or C₁₋₄ alkoxy, and the heterocycloalkyl contains 1 to 4 heteroatoms selected from O, S or N;
R^{1a} is selected from 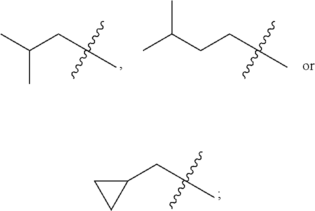
R^{b1} is selected from H, C₁₋₄ alkyl or C₃₋₆ cycloalkyl, wherein the alkyl or cycloalkyl is optionally substituted with 0 to 4 substituents selected from deuterium, halogen, OH, CN, NHC₁₋₄ alkyl, N(C₁₋₄ alkyl)₂, C₁₋₄ alkyl or C₁₋₄ alkoxy;
R^{b2} is selected from H, R^{b}a, or one of the following substituted or unsubstituted groups: C₁₋₄ alkyl, C₂₋₄ alkynyl, C₁₋₄ alkoxy, phenyl, 5- to 6-membered heteroaryl, —CH₂CH₂-phenyl, —CH₂CH₂-5- to 6-membered heteroaryl, —CH₂CH₂—NHC₁₋₄ alkyl, —CH₂CH₂—N(C₁₋₄ alkyl)₂, —CH₂CH₂—C₃₋₆ cycloalkyl or —CH₂CH₂-3- to 7-membered heterocycloalkyl, wherein the CH₂, alkyl, cycloalkyl or heterocycloalkyl is optionally substituted with 0 to 4 substituents selected from deuterium, halogen, OH, ═O, cyano, COOH, NH₂, NHC₁₋₄ alkyl, N(C₁₋₄ alkyl)₂, N(C₁₋₄ alkyl)(C₃₋₆ cycloalkyl), NH(C₃₋₆ cycloalkyl), C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, and the heteroaryl or heterocycloalkyl contains 1 to 4 heteroatoms selected from O, S or N;
R^{ba} is selected from —C₀₋₂ alkyl-7- to 8-membered monocyclic heterocycloalkyl, —C₀₋₂ alkyl-7- to 11-membered spiro heterocycloalkyl, —C₀₋₂ alkyl-7- to 11-membered bridged heterocycloalkyl, —C₀₋₂ alkyl-4- to 6-membered monocyclic heterocycloalkyl connected via a carbon atom, 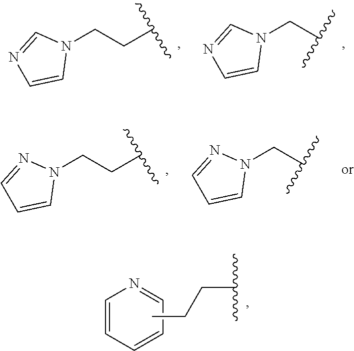
and the R^{ba} is optionally substituted with 1 to 4 substituents selected from H, halogen, OH, ═O, cyano, COOH, CONH₂, CONHC₁₋₄ alkyl, CON(C₁₋₄ alkyl)₂, NH₂, NHC₁₋₄ alkyl, N(C₁₋₄ alkyl)₂, C₁₋₄ alkyl, C₂₋₄ alkynyl, C₁₋₄ alkoxy, halogen-substituted C₁₋₄ alkyl, halogen-substituted C₁₋₄ alkoxy, C₁₋₄ alkoxyalkyl or R^{k}, wherein the heterocycloalkyl contains 1 to 4 heteroatoms selected from O, S or N;
R^{k} is selected from —C₁₋₂ alkyl-NH₂, —C₁₋₂ alkyl-NHC₁₋₄ alkyl, —C₁₋₂ alkyl-N(C₁₋₄ alkyl)₂, —C₀₋₂ alkyl-C₃₋₆ carbocyclic ring or —C₀₋₂ alkyl-3- to 6-membered heterocyclic ring, wherein the alkyl, carbocyclic ring or heterocyclic ring is optionally substituted with 1 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy, halogen-substituted C₁₋₄ alkyl, halogen-substituted C₁₋₄ alkoxy or C₁₋₄ alkoxyalkyl, and the heterocyclic ring contains 1 to 4 heteroatoms selected from O, S or N;
R³ is selected from H or C₁₋₄ alkyl, wherein the alkyl is optionally substituted with 0 to 4 substituents selected from deuterium, halogen, OH, ═O, cyano, COOH, NH₂, NHC₁₋₄ alkyl, N(C₁₋₄ alkyl)₂, C₁₋₄ alkyl, C₁₋₄ alkoxy or C₃₋₆ carbocyclic ring.

10. The compound or the stereoisomer, mesomer/racemate, deuterated product, solvate, prodrug, metabolite, pharmaceutically acceptable salt, or co-crystal thereof according to claim 9, wherein
R^{ak} is selected from ethynyl, propynyl or propargyl;
R^{a5} is selected from F, Cl, Br, I, OH, cyano, methyl, ethyl, methoxy, ethoxy or ethynyl.

11. The compound or the stereoisomer, mesomer/racemate, deuterated product, solvate, prodrug, metabolite, pharmaceutically acceptable salt, or co-crystal thereof according to claim 1, wherein the compound is selected from one of the structures shown in Table E-1, Table E-3, Table E-4 or Table E-5.

12. A pharmaceutical composition, comprising the compound or the stereoisomer, mesomer/racemate, deuterated product, solvate, prodrug, metabolite, pharmaceutically acceptable salt, or co-crystal thereof according to claim 1, and a pharmaceutically acceptable carrier, wherein preferably, the pharmaceutical composition comprises 1-1500 mg of the compound or the stereoisomer, mesomer/racemate, deuterated product, solvate, prodrug, metabolite, pharmaceutically acceptable salt, or co-crystal thereof.

13. (canceled)

14. (canceled)

15. A method for treating a disease in a mammal, the method comprising administering to a subject a therapeutically effective amount of the compound or the stereoisomer, mesomer/racemate, deuterated product, solvate, prodrug, metabolite, pharmaceutically acceptable salt, or co-crystal thereof according to claim 1.

16. The method according to claim 15, wherein the disease is a disease related to α4β7 activity or expression.

17. The method according to claim 15, wherein the therapeutically effective amount is 1-1500 mg.

18. The method according to claim 15, wherein the disease is inflammatory bowel disease.
